(19) United States
(12) Patent Application Publication
(10) Pub. No.: US 2026/0055079 A1
(43) Pub. Date: Feb. 26, 2026

(54) PHTHALAZINONE COMPOUNDS AS PARP7 INHIBITORS

(71) Applicants: AZKARRA THERAPEUTICS, INC., South San Francisco, CA (US); OREGON HEALTH & SCIENCE UNIVERSITY, Portland, OR (US)

(72) Inventors: Michael G. JOHNSON, San Francisco, CA (US); David C. SPELLMEYER, Oakland, CA (US); Raymond A. NG, Pleasant Hill, CA (US); David LAPOINTE, Oakland, CA (US); Jinxia N. DENG, Belmont, CA (US); Michael S. COHEN, Portland, OR (US); Kelsie M. RODRIGUEZ, Santa Cruz, CA (US); Sunil K. SUNDALAM, Santa Clara, CA (US); Daniel J. SANDERSON, Portland, OR (US); Guillaume PELLETIER, Sanit-Lazare (CA); Dana K. WINTER, Rigaud (CA); Polina NOVOSELTSEVA, Montreal (CA); Yuchen ZHOU, Pointe-Claire (CA)

(21) Appl. No.: 18/853,316

(22) PCT Filed: Mar. 31, 2023
(86) PCT No.: PCT/US2023/065214

§ 371 Date: Oct. 1, 2024

Related U.S. Application Data
(60) Provisional application No. 63/326,484, filed on Apr. 1, 2022.

(51) Int. Cl. C07D 401/12; A61K 31/502; A61K 31/5025; A61K 31/506; A61K 31/55; A61K 31/551; C07B 59/00; C07D 237/32; C07D 401/14; C07D 403/12; C07D 403/14; C07D 405/14; C07D 413/06; C07D 413/12; C07D 413/14; C07D 471/04; C07D 471/10; C07D 487/04; C07D 487/08; C07D 487/10

(52) U.S. Cl. CPC C07D 401/12; A61K 31/502; A61K 31/5025; A61K 31/506; A61K 31/55; A61K 31/551; C07B 59/002; C07D 237/32; C07D 401/14; C07D 403/12; C07D 403/14; C07D 405/14; C07D 413/06; C07D 413/12; C07D 413/14; C07D 471/04; C07D 471/10; C07D 487/04; C07D 487/08; C07D 487/10

(57) ABSTRACT

The present disclosure relates to phthalazinone compounds and related compounds and their use in treating a disease or condition responsive to inhibition of PARP7.

### CROSS-REFERENCE TO RELATED APPLICATIONS

This application claims priority to International PCT Application No. PCT/US23/65214, filed on Mar. 31, 2023, which claims the benefit of U.S. Provisional Application Ser. No. 63/326,484, filed on Apr. 1, 2022 and entitled “Phthalazinone Compounds as PARP7 Inhibitors,” the entire contents of which are incorporated herein by reference.

### STATEMENT OF GOVERNMENT INTEREST

This invention was made with government support under 2R01NS088629 awarded by the National Institutes of Health. The government has certain rights in this invention.

### SEQUENCE LISTING

The instant application contains a Sequence Listing that has been submitted in ASCII format via EFS-Web. The ASCII copy, created on Sep. 29, 2025, is named 98662.2.USW1 Sequence Listing.xml and is 8,964 bytes in size.

### FIELD OF DISCLOSURE

The present disclosure relates to compounds comprising a phthalazinone core, their use for modulating or inhibiting Poly (ADP-ribose) polymerase 7 (PARP7), and their use in pharmaceutical formulations.

### BACKGROUND

In humans there are 17 members of the PARP family of enzymes that catalyze the transfer of ADP-ribose from nicotinamide adenine dinucleotide (NAD⁺) to amino acids on protein targets of post-translational modification (PTM). PARP7 is a member of the PARP family that catalyzes PTM known as mono-ADP-ribosylation (MARylation) as opposed to the poly-ADP-ribosylation (PARylation) effected by other PARPs such as PARP1 and PARP2. Multiple independent lines of evidence point to PARP7 catalytic activity as a regulator of interferon signaling. In mouse embryonic fibroblasts (MEFs), knockout of PARP7 increases the type I interferon, interferon-beta (IFN-β), and synergizes with pattern recognition receptor (PRR) ligands (e.g. 3pRNA, agonist for RIG-1) to induce IFN-s production in cells. IFN-s has antitumor effects where it plays a role in dendritic cell (DC) driven T cell responses to various cancers. Hence, in cancers that overexpress PARP7 or that otherwise have dysregulated PARP7 activity, inhibition of PARP7 may increase IFN-β in the presence of PRR ligands, which could lead to immunogenic cell death and long-term protective antitumor immunity. Therefore, modulating or inhibiting PARP is a potential therapeutic approach for treating disorders such as cancer. Thus, there is a need for compounds that can modulate or inhibit PARP.

### SUMMARY

The present disclosure provides a compound of Formula I:

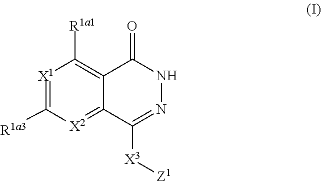

and stereoisomers and pharmaceutically acceptable salts thereof, wherein the constituent members are defined herein throughout the disclosure.

The present disclosure further provides a pharmaceutical composition comprising a compound of the present disclosure, and stereoisomers and pharmaceutically acceptable salts thereof, and a pharmaceutically acceptable carrier.

The present disclosure further provides a compound of the present disclosure, and stereoisomers and pharmaceutically acceptable salts thereof, for use in the treatment of a disorder that is responsive to inhibition of PARP7.

The present disclosure further provides for use of a compound of the present disclosure, and stereoisomers and pharmaceutically acceptable salts thereof, in the treatment of a disorder that is responsive to inhibition of PARP7.

The present disclosure further provides a compound of the present disclosure, and stereoisomers and pharmaceutically acceptable salts thereof, for use in the manufacture of a medicament for the treatment of a disorder that is responsive to inhibition of PARP7.

The present disclosure further provides a method of treating a disorder in a subject in need thereof, wherein the disorder is mediated by PARP7, comprising administering to the subject a compound of the present disclosure.

### DESCRIPTION OF THE FIGURES

The patent or application file contains at least one drawing executed in color. Copies of this patent or patent application publication with color drawing(s) will be provided by the Office upon request and payment of the necessary fee. The present application can be understood by reference to the following description taking in conjunction with the accompanying figures.

FIGS. 1A-E provide an overview of the rational design of Example 10, a potent membrane-permeable PARP7 inhibitor. FIG. 1A provides chemical structures of Phthal01. Example 10, and RBN-2397. Example 10 contains a propynyl group (shown in red) at the C-6 of the phthalazinone scaffold designed to impart PARP7-selectivity by interacting with a unique hydrophobic cavity in PARP7. FIG. 1B provides results of a family-wide PARP inhibitor screening assay that reveals that Example 10 is selective for PARP7. Selectivity data (IC₅₀ values) is represented as a heat map (red=more potent; and blue=less potent). IC₅₀ values for the data provided in the heat map can be found in Table 4 and individual dose response curves in FIGS. 11 and 12. FIG. 1C provides fit homology model of Example 10 (orange) bound to PARP7 and shows the propynyl group occupying the hydrophobic cavity formed by ile631 and hydrophobic amino acids in the D-loop (cyan). The induced fit homology model of RBN-2397 (steel blue) shows that the methyl substituent only partially occupies the hydrophobic cavity. FIG. 1D provides data showing Example 10 inhibits GFP-PARP7 auto-MARylation in a dose-dependent manner in HEK 293T cells. GFP-PARP7 levels and MARylation were detected by Western blot analysis using an anti-GFP antibody and an anti-M/PAR antibody, respectively. Replicates shown in FIG. 8. FIG. 1E provides quantification of replicates in FIG. 1D and FIG. 8 using BioRad ImageLab software. Curves fit in Graphpad Prism with four parameter nonlinear regression.

FIGS. 2A-D demonstrate inhibition of PARP7 catalytic activity by Example 10 derepresses AHR ligand-mediated gene transcription in mouse embryonic fibroblasts (MEFs). FIG. 2A provides CYP1A1 mRNA levels assessed by qPCR after treatment of wild type (WT) MEFs with increasing concentrations of Example 10 in the presence (gray bars) or absence (black bars) of the AHR agonist TCDD (1 nM). FIG. 2B provides the same as in FIG. 2A except with PARP7^{-/-} MEFs. FIG. 2C provides CYP1A1 mRNA levels assessed by qPCR after treatment of wild type (WT) MEFs with increasing concentrations of TCDD in the presence (gray bars) or absence (black bars) of 100 nM Example 10. FIG. 2D provides the same as in FIG. 2C, except with PARP7−/− MEFs.

FIGS. 3A-C demonstrate Example 10 enhances 3pRNA- and cGAMP-stimulated IFN-β transcription. FIG. 3A shows MEFs were treated with 100 nM Example 10 (black bar) or DMSO (white bar) for 24 h. IFN-β mRNA levels were measured using quantitative RT-PCR. IFN-β levels were normalized to expression of TBP mRNA levels. FIG. 3B shows MEFs were treated with the RIG-I ligand 3pRNA (100 ng/ml) in the presence (black bar) or absence (gray bar) of 100 nM Example 10 for 4 h. IFN-β mRNA levels were assessed as in FIG. 3A. FIG. 3C shows MEFs were treated with the STING ligand cGAMP (5 mg/ml) in the presence (black bar) or absence (gray bar) of 100 nM Example 10 for 4 h. IFN-β mRNA levels were assessed as in FIG. 3A.

FIGS. 4A-E demonstrate inhibition of PARP7 catalytic activity induces a STING-dependent type I interferon response and PARP7 protein accumulation in CT-26 cells. FIG. 4A demonstrate Example 10 and RBN-2397 increase STAT1 and phosphor-Tyr701-STAT1 (pSTAT1) in a dose-dependent manner. CT-26 cells were treated with increasing concentrations of either Example 10 or RBN-2397 for 16 h. FIG. 4B provides of Western blots in FIG. 4A. Quantification was performed using BioRad ImageLab software. Curves were fit using four parameter nonlinear regression in GraphPad Prism. STAT1 and pSTAT1 levels were normalized to tubulin. Data was from 3 biological replicates. FIG. 4C demonstrates Example 10 and RBN-2397 induced STING degradation and signaling, but not to the same extent as a STING agonist. Treatment was as in FIG. 4A. Note: tubulin blot was the same as in FIG. 4A since samples were run on the same nitrocellulose membrane for western blot analysis. SE=short exposure; LE=long exposure. FIG. 4D demonstrates Example 10 and RBN-2397 increase PARP7 levels in a dose-dependent manner. Treatment was as in FIG. 4A. Note: tubulin blot is the same as in FIG. 4A since samples were run on the same nitrocellulose membrane for western blot analysis. FIG. 4E demonstrates quantification of Western blots in FIG. 4D. Quantification was performed using BioRad ImageLab software. Curves fitting was done using a four parameter nonlinear regression in GraphPad Prism. PARP7 levels were normalized to tubulin. Data was from 3 biological replicates. FIG. 4F demonstrates Example 10 and RBN-2397 increase IFN-β levels in a STING-dependent manner. CT-26 cells were treated with 300 nM Example 10 (dark gray) or 300 nM RBN-2397 (light gray) in the presence or absence of the covalent STING inhibitor H-151 (500 nM) for 16 h. IFN-0 levels were determined by ELISA (PBL Assay Science). Statistical analysis was performed as one-way ANOVA in GraphPad Prism. ****=p<0.001. FIG. 4G demonstrates Example 10 and RBN-2397 increase type I interferon reporter levels in a dose dependent manner. CT-26 cells stably expressing an IRSE luciferase reporter were treated with increasing concentrations of Example 10 or RBN-2397 for 16 h. Luciferase levels were measured using a luciferase assay reagent (Promega).

FIGS. 5A-D demonstrate PARP7 catalytic activity controls its protein abundance in the nucleus. FIG. 5A demonstrates inhibition of PARP7 with either Example 10 or RBN-2397 increases nuclear PARP7 protein levels. HiBiT-PARP7 knockin (KI) CT-26 cells were treated with 300 nM KMR, 300 nM RBN-2397, or DMSO for 18 h. WT CT-26 cells were used as a control. HiBiT-PARP7 levels were determined by immunofluorescence staining with an anti-HiBiT antibody followed by an anti-mouse AlexaFluor647 antibody. Nuclei were stained with DAPI. Scale bar=25 μm. FIG. 5B provides quantification of DAPI and AlexaFluor647 fluorescence in FIG. 5A across linear trace of nuclei. Replicate traces across RBN-2397 and Example 10 treated cells presented in FIG. 11. FIG. 5C demonstrates PARP7 levels were only detected in nucleus of CT-26 cells treated with PARP7 inhibitors. CT-26 cells were treated with 300 nM Example 10 or 300 nM RBN-2397 for 18 h. Cells were fractioned and PARP7 levels were detected by Western blot using an anti-PARP7 antibody. An anti-PARP-1 antibody was used as a nuclear marker whereas an anti-GAPDH was used as a cytosolic maker. FIG. 5D demonstrates increases in nuclear PARP7 levels were be detected within 30 min of PARP7 inhibitor treatment. HeLa cells overexpressing GFP-PARP7 and mRuby2-Nup90 (nuclear pore marker) were treated with 300 nM Example 10, 300 nM RBN-2397, or DMSO and live cell images were taken at 0, 30, and 120 min. Representative images shown. Scale bar=20 μm.

FIG. 6A provides individual dose response curves for Example 10 across the PARP family. FIG. 6B further provides individual dose response curves for Example 10 across the PARP family.

FIG. 7A provides individual dose response curves for RBN-2397 across the PARP family. FIG. 7B further provides individual dose response curves for RBN-2397 across the PARP family.

FIG. 8 provides replicate blots of FIG. 1D. HEK 293T cells were transfected with GFP-PARP7 and treated with increasing concentrations of Example 10 PARP7 for 18 h. GFP-PARP7 auto-MARylation and GFP-PARP7 protein levels were determined by Western blot using specific antibodies.

FIG. 9 demonstrates PARP7 inhibition decreases viability in NCI-H1373 cells. Cell viability in NCI-H1373 cells was measured by Cell Titer-Glo assay following a 6 d treatment with increasing concentrations of Example 10 or RBN-2397. Curves were fit using three parameter a nonlinear regression in GraphPad Prism.

FIG. 10A-D demonstrates PARP7 inhibition, but not Type I interferons drives PARP7 protein accumulation and IFN-I signaling in a time-dependent manner. FIG. 10A demonstrates Example 10 and RBN-2397 increase STAT1 and pSTAT1 in a time-dependent manner. CT-26 cells were treated with 300 nM Example 10 or RBN-2397 and cells were harvested for western blot analysis at the indicated times. FIG. 10B demonstrates Example 10 and RBN-2397 decrease STING levels and increase pIRF3 levels in a time-dependent manner. Treatment was same as in FIG. 10A. Note: tubulin blot is the same as in FIG. 10A since samples were run on the same nitrocellulose membrane for western blot analysis. FIG. 10C demonstrates Example 10 and RBN-2397 increase PARP7 levels in a time-dependent manner. Treatment was same as in FIG. 10A. Note: tubulin blot is the same as in FIG. 10A since samples were run on the same nitrocellulose membrane for western blot analysis. FIG. 10D shows results of CT-26 cells treated with indicated compounds, IFN-β (100 U/ml), or DMSO for 18 h. Proteins were detected by Western blot using specific antibodies.

FIG. 11 provides replicate cell traces from HiBiT-PARP7 CT-26 cells in FIG. 4 treated with 0.3 μM RBN-2397 or Example 10. Traces were drawn and measured with ImageJ software. Plots were generated in GraphPad Prism.

FIG. 12 provides synthesis of Example 10 (a) NBS, AIBN, CCl₄ reflux; (b) PPh₃, THF reflux; (c) 1) 2-fluoro-5-formylbenzonitrile, TEA, DCM 2) Hydrazine hydrate, H₂O, EtOH, DMF reflux; (d) 1) KOH. EtOH, ddH₂O, 2) HCl; (e) 6-(piperizino)pyridine-3-carbonitrile, propanephosphonic acid anhydride (T3P®), DIPEA, DMF; (f) tributyl(1-propynyl)tin, Pd(PPh₃)₄, toluene reflux.

### DETAILED DESCRIPTION

The following description sets forth numerous exemplary configurations, methods, parameters, and the like. It should be recognized, however, that such description is not intended as a limitation on the scope of the present disclosure, but is instead provided as a description of exemplary embodiments.

As used herein, the terms “including,” “containing,” and “comprising” are used in their open, non-limiting sense.

The articles “a” and “an”, as used herein, refer to one or more than one (i.e., to at least one) of the grammatical object of the article. By way of example. “an element” refers to one element or more than one element.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

“Alkyl”, as used herein, refers to an unbranched or branched saturated hydrocarbon chain. Alkyl can be used alone, or as part of another radical, such as —O-alkyl. In some embodiments, alkyl as used herein has 1 to 20 carbon atoms ((C₁₋₂₀)alkyl), 1 to 12 carbon atoms ((C₁₋₁₂)alkyl), 1 to 10 carbon atoms ((C₁₋₁₀)alkyl), 1 to 8 carbon atoms ((C₁₋₈)alkyl), 1 to 6 carbon atoms ((C₁₋₆)alkyl), 1 to 4 carbon atoms ((C₁₋₄)alkyl), or 1 to 3 carbon atoms ((C₁₋₃)alkyl). Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methyl pentyl. When an alkyl residue having a specific number of carbons is named, all geometric isomers having that number of carbons may be encompassed. Thus, for example, “butyl” can include n-butyl, sec-butyl, isobutyl and t-butyl, and “propyl” can include n-propyl and isopropyl.

“Alkenyl”, as used herein, refers to an unbranched or branched hydrocarbon chain. The “alkenyl” group contains at least one double bond. The double bond of an alkenyl group can be unconjugated or conjugated to another group. The alkenyl may be branched or straight. In some embodiments, alkenyl as used herein has 2 to 20 carbon atoms ((C₂₋₂₀)alkenyl), 2 to 12 carbon atoms ((C₂₋₁₂)alkenyl), 2 to 10 carbon atoms ((C₂₋₁₀)alkenyl), 2 to 8 carbon atoms ((C₂₋₈)alkenyl), 2 to 6 carbon atoms ((C₂₋₆)alkenyl, 2 to 4 carbon atoms ((C₂₋₄)alkenyl), or 2 to 3 carbon atoms ((C₂₋₃)alkenyl). Examples of alkenyl groups include, but are not limited to, ethylenyl, vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl and the like. When an alkenyl residue having a specific number of carbons is named, all geometric isomers and all E-Z isomers having that number of carbons may be encompassed.

“Alkynyl”, as used herein, refers to an unbranched or branched unsaturated hydrocarbon chain. The “alkynyl” group contains at least one triple bond. The alkynyl may be branched or straight. The triple bond of an alkynyl group can be unconjugated or conjugated to another group. In some embodiments, alkynyl as used herein has 2 to 50 carbon atoms ((C₂₋₅₀)alkynyl), 2 to 20 carbon atoms ((C₂₋₂₀)alkynyl), 2 to 12 carbon atoms ((C₂₋₁₂)alkynyl), 2 to 10 carbon atoms ((C₂₋₁₀)alkynyl), 2 to 8 carbon atoms ((C₂₋₈)alkynyl), 2 to 6 carbon atoms ((C₂₋₆)alkynyl, 2 to 4 carbon atoms ((C₂₋₄)alkynyl), or 2 to 3 carbon atoms ((C₂₋₃)alkynyl). Examples of alkynyl groups include, but are not limited to, ethynyl, propynyl, butynyl, pentynyl, hexynyl, methylpropynyl, 4-methyl-1-butynyl, 4-propyl-2-pentynyl, 4-butyl-2-hexynyl and the like. When an alkynyl residue having a specific number of carbons is named, all geometric isomers having that number of carbons may be encompassed.

“Cycloalkyl”, as used herein, refers to a saturated or partially saturated, monocyclic, fused or spiro polycyclic, carbocycle having from 3 to 18 carbon atoms per ring. The cycloalkyl ring or carbocycle may be unsubstituted or substituted by one or more substituents, e.g., 1 to 5 substituents, at any point of attachment. The substituents can themselves be unsubstituted or substituted. Examples of cycloalkyl groups include, without limitations, cyclopropyl, cyclobutyl, cyclopentyl, cyclohexyl, cycloheptanyl, cyclooctanyl, norboranyl, norborenyl, bicyclo[2,2,2]octanyl, bicyclo[2,2,2]octenyl, decahydronaphthalenyl, octahydro-1H-indenyl, cyclopentenyl, cyclohexenyl, cyclohexa-1,4-dienyl, cyclohexa-1,3-dienyl, 1,2,3,4-tetrahydronaphthalenyl, octahydropentalenyl, 3a,4,5,6,7,7a-hexahydro-1H-indenyl, 1,2,3,3a-tetrahydropentalenyl, bicyclo[3.1.0]hexanyl, bicyclo[2.1.0]pentanyl, spiro[3.3]heptanyl, bicyclo[2.2.1]heptanyl, bicyclo[2.2.1]hept-2-enyl, bicyclo[2.2.2]octanyl, 6-methylbicyclo[3.1.1]heptanyl, 2,6,6-trimethylbicyclo[3.1.1]heptanyl, and derivatives thereof.

“Cycloalkenyl”, as used herein, refers to a partially saturated, monocyclic or fused or spiro polycyclic carbocycle having from 3 to 18 carbon atoms per ring and containing at least one double bond. The cycloalkenyl ring may be unsubstituted or substituted by one or more substituents, e.g., 1 to 5 substituents, at any point of attachment. The substituents can themselves be unsubstituted or substituted.

“Heterocycle”, “heterocyclyl”, or “heterocyclediyl”, as used herein, refers to a saturated or partially unsaturated and non-aromatic monocyclic or fused polycyclic or spiro polycyclic ring structure of 4-to-18 atoms containing carbon and heteroatoms taken from oxygen, nitrogen, or sulfur wherein there is not delocalized π-electrons (aromaticity) shared among all ring carbons or heteroatoms. A heterocyclyl ring structure attaches to a single point of a moiety of the formulae described herein, while a heterocyclediyl ring structure attaches to two points of a moiety or moieties of formulae described herein. The heterocycle, heterocyclyl, or heterocyclediyl ring structure may be unsubstituted or substituted by one or more substituents. The substituents can themselves be unsubstituted or substituted. Examples of heterocycle, heterocyclyl, or heterocyclediyl rings include, but are not limited to, oxetanyl, azetidinyl, tetrahydrofuranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, homotropanyl, dihydrothiophen-2(3H)-onyl, tetrahydrothiophene 1,1-dioxide, 2,5-dihydro-1H-pyrrolyl, imidazolidin-2-one, pyrrolidin-2-one, dihydrofuran-2(3H)-one, 1,3-dioxolan-2-one, isothiazolidine 1,1-dioxide, 4,5-dihydro-1H-imidazolyl, 4,5-dihydrooxazolyl, oxiranyl, pyrazolidinyl, 4H-1,4-thiazinyl, thiomorpholinyl, 1,2,3,4-tetrahydropyridinyl, 1,2,3,4-tetrahydropyrazinyl, 1,3-oxazinan-2-one, tetrahydro-2H-thiopyran 1,1-dioxide, 7-oxabicyclo[2.2.1]heptanyl, 1,2-thiazepane 1,1-dioxide, octahydro-2H-quinolizinyl, 1,3-diazabicyclo[2.2.2]octanyl, 2,3-dihydrobenzo[b][1.4]dioxine, 3-azabicyclo[3.2.1]octanyl, 8-azaspiro[4.5]decane, 8-oxa-3-azabicyclo[3.2.1]octanyl, 2-azabicyclo[2.2.1]heptane, 2,8-diazaspiro[5.5]undecanyl, 2-azaspiro[5.5]undecanyl, 3-azaspiro[5.5]undecanyl, decahydroisoquinolinyl, 1-oxa-8-azaspiro[4.5]decanyl, 8-azabicyclo[3.2.1]octanyl, 1,4′-bipiperidinyl, azepanyl, 8-oxa-3-azabicyclo[3.2.1]octanyl, 3,4-dihydro-2H-benzo[b][1,4]oxazinyl, 5,6,7,8-tetrahydroimidazo[1,2-a]pyridinyl, 1,4-diazepanyl, phenoxathiinyl, benzo[d][1,3]dioxolyl, 2,3-dihydrobenzofuranyl, 2,3-dihydrobenzo[b][1,4]dioxinyl, 4-(piperidin-4-yl)morpholinyl, 3-azaspiro[5.5]undecanyl, decahydroquinolinyl, piperazin-2-one, 1-(pyrrolidin-2-ylmethyl)pyrrolidinyl, 1,3′-bipyrrolidinyl, and 6,7,8,9-tetrahydro-1H,5H-pyrazolo[1,2-a][1.2]diazepinyl.

“Aryl”, as used herein, refers to a monocyclic or polycyclic group having at least one hydrocarbon aromatic ring wherein all of the ring atoms of the at least one hydrocarbon aromatic ring are carbon. Aryl may include groups with a single aromatic ring (e.g., phenyl) and multiple fused aromatic rings (e.g., naphthyl, anthryl). Aryl may further include groups with one or more aromatic hydrocarbon rings fused to one or more non-aromatic hydrocarbon rings (e.g., fluorenyl; 2,3-dihydro-1H-indene; 1,2,3,4-tetrahydronaphthalene). In certain embodiments, aryl includes groups with an aromatic hydrocarbon ring fused to a non-aromatic ring wherein the non-aromatic ring comprises at least one ring hetero atom independently selected from the group consisting of N, O, and S. For example, in some embodiments, aryl includes groups with a phenyl ring fused to a non-aromatic ring, wherein the non-aromatic ring comprises at least one ring hetero atom independently selected from the group consisting of N. O, and S (e.g., chromane; thiochromane; 2,3-dihydrobenzofuran; indoline). In some embodiments, aryl as used herein has from 6 to 14 carbon atoms ((C₆-C₁₄)aryl), or 6 to 10 carbon atoms ((C₆-C₁₀)aryl). Where the aryl includes fused rings, the aryl may connect to one or more substituents or moieties of the formulae described herein through any atom of the fused ring for which valency permits.

“Heteroaryl”, as used herein, refers to a monocyclic or polycyclic group comprising at least one aromatic ring, wherein the aromatic ring comprises at least one ring heteroatom independently selected from the group consisting of N. O, and S. The heteroaryl group may comprise 5, 6, 7, 8, 9, 10, 11, 12, or more ring atoms, where ring atoms refer to the sum of carbon and heteroatoms in the one or more rings (e.g., be a 5-membered, 6-membered, 7-membered, 8-membered, 9-membered, 10-membered, 11-membered, or 12-membered heteroaryl). In some embodiments, heteroaryl includes groups with an aromatic ring that comprises at least one ring heteroatom independently selected from the group consisting of N, O, and S. (e.g., pyridinyl, pyrazinyl, furanyl, thiophenyl). In certain embodiments, heteroaryl includes polycyclic groups with an aromatic ring comprising at least one ring heteroatom, fused to a non-aromatic hydrocarbon ring (e.g., 5,6,7,8-tetrahydroquinolinyl; 4,5,6,7-tetrahydroisobenzofuranyl). In some embodiments, heteroaryl includes polycyclic groups with an aromatic ring comprising at least one ring heteroatom fused to an aromatic hydrocarbon ring (e.g., quinolinyl, quinoxalinyl, benzothiazolyl). In still further embodiments, heteroaryl includes polycyclic groups with two fused aromatic rings, wherein each ring comprises at least one ring heteroatom (e.g., naphthyridinyl). Heteroaryl may include groups comprising 1 to 5 ring heteroatoms, 1 to 4 ring heteroatoms, 1 to 3 ring heteroatoms, 1 or 2 ring heteroatoms, or 1 ring heteroatom, wherein each ring heteroatom is independently selected from the group consisting of N. O, and S. In one example, a heteroaryl has 3 to 8 ring carbon atoms, with 1 to 3 ring heteroatoms independently selected from N. O, and S. Examples of heteroaryl groups include, without limitations, pyridyl, pyridazinyl, pyrimidinyl, benzothiazolyl, and pyrazolyl.

As used herein, the term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein, the term “unsubstituted” means that the specified group bears no substituents.

“Amino”, as used herein, means a substituent containing at least one nitrogen atom. For example, NH₂, —NH(alkyl) or alkylamino. —N(alkyl)₂ or dialkylamino, amide, carboxamide, urea, and sulfamide are included in the term “amino”.

“Cyano”, as used herein, refers to a substituent having a carbon atom joined to a nitrogen atom by a triple bond, i.e., C≡N.

“Hydroxyl” or “hydroxy”, as used herein, refers to an OH group.

“Halogen” or “halo”, as used herein, refers to fluoro, chloro, bromo, or iodo radicals.

“Haloalkyl,” as used herein, refers to an alkyl group substituted with one or more halogen.

“Halocycloalkyl”, as used herein, refers to a cycloalkyl group substituted with one or more halogen.

“Haloaryl”, as used herein, refers to an aryl group substituted with one or more halogen.

“Oxo”, as used herein, refers to an “═O” group.

It should be understood that when a range of values is listed, it is intended to encompass each value and sub-range within the range. For example, “C₁₋₆alkyl” (which may also be referred to as C1-C6 alkyl, C₁-C₆ alkyl, or C₁₋₆ alkyl) is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

It should also be noted that any carbon as well as heteroatom with unsatisfied valences in the text, schemes, examples and tables herein is assumed to have the sufficient number of hydrogen atom(s) to satisfy the valences.

As used herein, references to hydrogen may also refer to a deuterium substitution if desired. The term “deuterium” as used herein means a stable isotope of hydrogen having odd numbers of protons and neutrons.

Compounds of the various Formulae and stereoisomers and pharmaceutically acceptable salts thereof may exist in their tautomeric form (for example, as an amide or imino ether). All such tautomeric forms are contemplated herein as part of the present disclosure.

It should be understood that all isomeric forms are included within the present disclosure, including mixtures thereof. If the compound contains a double bond, the substituent may be in the E or Z configuration. If the compound contains a disubstituted cycloalkyl, the cycloalkyl substituent may have a cis- or trans-configuration.

The compounds of the various Formulae may contain asymmetric or chiral centers, and, therefore, exist in different stereoisomeric forms. It is intended that all stereoisomeric forms of the compounds of the various Formulae as well as mixtures thereof, including racemic mixtures, form part of the present disclosure. In some embodiments, isomers of the compounds herein are stereoisomers. In addition, the present disclosure embraces all geometric and positional isomers. For example, if a compound of the various Formulae incorporates a double bond or a fused ring, both the cis- and trans-forms, as well as mixtures, are embraced within the scope of the present disclosure. Each compound herein disclosed includes all the enantiomers that conform to the general structure of the compound. The compounds may be in a racemic or enantiomerically pure form, or any other form in terms of stereochemistry. The assay results may reflect the data collected for the racemic form, the enantiomerically pure form, or any other form in terms of stereochemistry.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers, and converting (e.g., hydrolyzing) the individual diastereomers to the corresponding pure enantiomers. Also, some of the compounds of the various Formulae may be atropisomers (e.g., substituted biaryls) and are considered as part of the present disclosure. Enantiomers can also be separated by use of a chiral HPLC column.

In some embodiments, the compounds of Formulae I, II, III, IV, V, VI, and VII and pharmaceutically acceptable salts thereof are enantiomers. In some embodiments, the compounds and pharmaceutically acceptable salts thereof are the (S)-enantiomer. In other embodiments the compounds and pharmaceutically acceptable salts thereof are the (R)-enantiomer. In some embodiments, the compounds and pharmaceutically acceptable salts thereof are the (+) enantiomer or (−) enantiomer.

Some embodiments are directed to isotopically-labelled compounds of the present disclosure which are identical to those recited herein but for the fact that one or more atoms are replaced by an atom having an atomic mass or mass number different from the atomic mass or mass number usually found in nature. Examples of isotopes that can be incorporated into compounds of the present disclosure include isotopes of hydrogen, carbon, nitrogen, oxygen, phosphorus, fluorine and chlorine, such as ²H (or D), ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of the various Formulae (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of the various Formulae can generally be prepared by following procedures analogous to those disclosed in the Schemes and/or in the Examples herein below by substituting an appropriate isotopically labelled reagent for a non-isotopically labelled reagent.

In some embodiments, the compound comprises at least one deuterium atom. For example, one or more hydrogen atoms in a compound of the present disclosure can be replaced or substituted by deuterium. In some embodiments, the compound comprises two or more deuterium atoms. In some embodiments, the compound comprises 1, 2, 3, 4, 5, 6, 7, 8, 9, 10, 11 or 12 deuterium atoms.

The compounds of Formulae I, II, III, IV, V, VI, and VII may form salts which are also within the scope of the present disclosure. Reference to a compound of the Formula herein is understood to include reference to salts thereof, unless otherwise indicated.

The present disclosure is directed to compounds as described herein and stereoisomers and pharmaceutically acceptable salts thereof. The present disclosure is also directed to pharmaceutical compositions comprising one or more compounds as described herein and stereoisomers and pharmaceutically acceptable salts thereof.

“Pharmaceutically acceptable”, as used herein, refers to that which is useful in preparing a pharmaceutical composition that is generally safe, non-toxic and not biologically or otherwise undesirable, and includes that which is acceptable for veterinary use as well as human pharmaceutical use. For example, provided herein is a pharmaceutical composition comprising a compound of Formulae I, II, III, IV, V, VI, or VII and stereoisomers and pharmaceutically acceptable salts thereof, and a pharmaceutically acceptable excipient.

“Pharmaceutically acceptable salt”, as used herein, refers to a salt which is generally safe, non-toxic and not biologically or otherwise undesirable, and includes that which is acceptable for veterinary use as well as human pharmaceutical use. Such salts may include acid addition salts and base addition salts. Acid addition salts may be formed with inorganic acid such as, but not limited to, hydrochloric acid, hydrobromic acid, sulfuric acid, nitric acid, phosphoric acid and the like; or an organic acid such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, or undecylenic acid. Salts derived from inorganic bases may include, but are not limited to, sodium, potassium, lithium, ammonium, calcium, magnesium, iron, zinc, copper, manganese, and aluminum salts. Salts derived from organic bases may include, but are not limited to, salts of primary, secondary, or tertiary amines, substituted amines including naturally occurring substituted amines; cyclic amines; ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, or N-ethylpiperidine.

The term “carrier”, as used herein, encompasses carriers, excipients, and diluents and refers to a material, composition, or vehicle such as a liquid or solid filler, diluent, excipient, solvent, or encapsulating material involved in carrying or transporting a pharmaceutical agent from one organ, or portion of the body, to another organ, or portion of the body, of a subject. Excipients should be selected on the basis of compatibility and the release profile properties of the desired dosage form. Exemplary carrier materials include. e.g., binders, suspending agents, disintegration agents, filling agents, surfactants, solubilizers, stabilizers, lubricants, wetting agents, diluents, spray-dried dispersions, and the like.

“Pharmaceutically compatible carrier materials” may include. e.g., acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, and the like. See, e.g., Hoover. John E., Remington's Pharmaceutical Sciences, Mack Publishing Co., Easton. Pa. 1975.

“Solvate”, as used herein, refers to a complex of variable stoichiometry formed by a solute and solvent. Such solvents for the purpose of the present disclosure may not interfere with the biological activity of the solute. Examples of suitable solvents include, but are not limited to, water, methanol, ethanol, and acetic acid. Solvates wherein water is the solvent molecule are typically referred to as hydrates. Hydrates include compositions containing stoichiometric amounts of water, as well as compositions containing variable amounts of water.

### Compounds

The present disclosure provides a compound of Formula I;

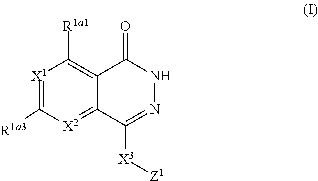

and stereoisomers and pharmaceutically acceptable salts thereof, wherein:
X¹ is —N— or —CR^{1a2}—;
X² is —N— or CR^{1a4}—;
R^{1a1}, R^{1a2}, R^{1a3}, and R^{1a4} are independently H, halo, —OH, C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, —SO₂-cycloalkyl, —NR^{1L1}R^{1L2}, or C₂₋₆alknyl-NR^{1L3}R^{1L4};
wherein if X¹ is —CR^{1a2}— and X² is —CR^{1a4}—, then at least one of R^{1a1}, R^{1a2}, R^{1a3}, and R^{1a4} is not H;
wherein each C₁₋₆alkyl, C₂₋₆alkenyl, C₂₋₆alkynyl, cycloalkyl, heterocyclyl, aryl, heteroaryl, —O—C₁₋₆ alkyl, —O—C₂₋₆alkenyl, —O—C₂₋₆alkynyl, —O-cycloalkyl, —O-heterocyclyl, —O-aryl, —O-heteroaryl, or —SO₂-cycloalkyl of R^{1a1}, R^{1a2}, R^{1a3}, and R^{1a4} is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂;
R^{1L1}, R^{1L2}, R^{1L3}, and R^{1L4} are independently H, C₁₋₆alkyl, C₂₋₆alkynyl, or cycloalkyl, wherein each C₁₋₆alkyl, C₂₋₆alkynyl, or cycloalkyl of R^{1L1}, R^{1L2}, R^{1L3}, and R^{1L4} is unsubstituted or substituted with one or more substituents independently selected from the group consisting of halo, C₁₋₆alkyl, C₁₋₆haloalkyl, cycloalkyl, and halocycloalkyl;
X³ is —O—, —NR^{1b1}—, or —CR^{1b2}R^{1b3}—, wherein R^{1b1}, R^{1b2}, and R^{1b3} are independently H or C₁₋₆alkyl;
Z¹ is

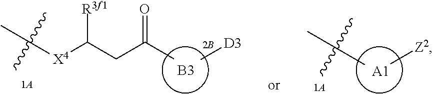

wherein a bond marked 1A is to X³ and a bond marked 2B is to D3, D4, D5, or D6;
R^{3f1} is H or C₁₋₆alkyl;
X⁴ is —O—, —NR^{3f2}—, or —CR^{3f3}R^{3f4}—, wherein R^{3f2}, R^{3f3}, and R^{3f4} are independently selected from H and C₁₋₆alkyl;
A1 is

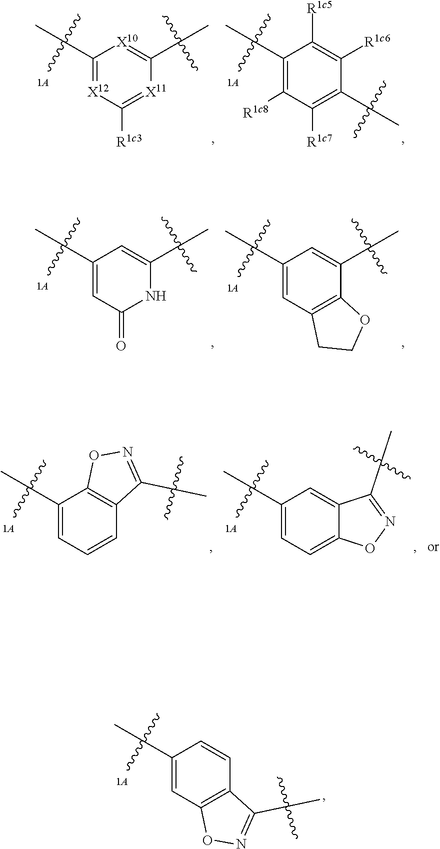

wherein a bond marked 1A is to X³;
X¹⁰ is —N— or —CR^{1c1}—, X¹¹ is —N— or —CR^{1c2}—, and X¹² is —N— or —CR^{1c4}—;
R^{1c1}, R^{1c2}, R^{1c3}, R^{1c4}, R^{1c5}, R^{1c6}, R^{1c7}, and R^{1c8} are independently H, halo, —CN, —SO₂CH₃, —SO₂NH₂, or —NHSO₂CH₃;
Z² is

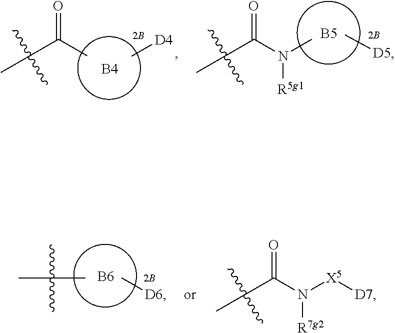

wherein a bond marked 2B is to D3, D4, D5, or D6;
X⁵ is —CH(R^{7g3})— or —CH(R^{7g4})CH₂N(R^{7g5})—;
R^{5g1}, R^{7g2}, R^{7g3}, R^{7g4}, and R^{7g5} are independently H or C₁₋₆alkyl;
B3, B4, B5, and B6 are independently a 3 to 8-membered monocyclic heterocyclediyl, a 7 to 18-membered polycyclic heterocyclediyl, or a 7 to 18-membered spirocyclic heterocyclediyl;
wherein the 3 to 8-membered monocyclic heterocyclediyl, 7 to 18-membered polycyclic heterocyclediyl, or 7 to 18-membered spirocyclic heterocyclediyl of B3, B4, B5, and B6 is unsubstituted or substituted with one or more substituents independently selected from the group consisting of halo, C₁₋₆alkyl, and oxo;
provided that B4 is not

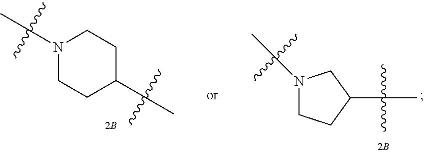 [SMILES: *C1CCN(*)CC1 ; *C1CCN(*)C1]

D3, D4, D5, D6, and D7 are independently C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, —C(O)-heteroaryl, —N(R^{1D1})(R^{1D2}), —C(O)N(R^{1D3})(R^{1D4}), or —N(R^{1D5})C(O)R^{1D6};
wherein the C₁₋₆alkyl, cycloalkyl, aryl, heteroaryl, —O—C₁₋₆alkyl, —O-aryl, —O-heteroaryl, —C(O)—C₁₋₆ alkyl, —C(O)-cycloalkyl, —C(O)-heterocyclyl, —C(O)-aryl, or —C(O)-heteroaryl of D3, D4, D5, D6, and D7 is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{1D10})₂, wherein each R^{1D10} is independently H or C₁₋₆alkyl;
R^{1D1}, R^{1D3}, and R^{1D5} are independently H or C₁₋₆alkyl;
R^{1D2} is aryl or heteroaryl, wherein the aryl or heteroaryl of R^{1D2} is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{1D11})₂, wherein each R^{1D11} is independently H or C₁₋₆alkyl; and
R^{1D4} and R^{1D6} are independently C₁₋₆alkyl, cycloalkyl, heterocyclyl, aryl, or heteroaryl, wherein each C₁₋₆alkyl, cycloalkyl, heterocyclyl, aryl, or heteroaryl of R^{1D4} and R^{1D6} is unsubstituted or substituted with one or more substituents independently selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{1D12})₂, wherein each R^{1D12} is independently H or C₁₋₆alkyl;
provided that if B4 is

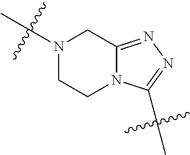 [SMILES: *c1nnc2n1CCN(*)C2]

and D4 is —CHF₂, —CF₂CH₃, or —CF₂CF₃, then R^{1a2} is not F, if X³ is —O—, B4 is

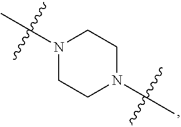 [SMILES: *N1CCN(*)CC1]

and D4 is —C(O)-aryl, then R^{1a4} is not Cl, and if D4 is

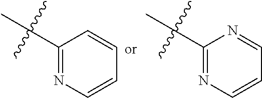 [SMILES: *c1ccccn1 ; *c1ncccn1]

and R^{1c2} is F, then R^{1a2} is not F.

In some embodiments of the compounds of Formula I and stereoisomers and pharmaceutically acceptable salts thereof. X¹ is —CR^{1a2}—. In some embodiments, X² is —CR^{1a4}—. In some embodiments, X¹ is —N—. In some embodiments, X² is —N—. In some embodiments, X¹ is —CR^{1a2}—. In some embodiments, X¹ is —CR^{1a2}— and X² is —N—. In some embodiments, X¹ is —N— and X² is —CR^{1a4}—.

In some embodiments of the compounds of Formula I and stereoisomers and pharmaceutically acceptable salts thereof, R^{1a2} is H, Cl, Br, I, —OH, C₁₋₆alkyl, C₂₋₆alkenyl, C₂₋₆-alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, —SO₂-cycloalkyl, —NR^{1L1}R^{1L2}, or C₂₋₆alkynyl-NR^{1L3}R^{1L4}, wherein each C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, or —SO₂-cycloalkyl of R^{4a2} is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂.

In some embodiments of the compounds of Formula I and stereoisomers and pharmaceutically acceptable salts thereof, R^{1a4} is H, F. Br, I, —OH, C₁₋₆alkyl, C₂₋₆alkenyl, C₂₋₆alkynyl, cycloalkyl, heterocyclyl, aryl, heteroaryl, —C₁₋₆alkyl, —O—C₂₋₆alkenyl, —O—C₂₋₆alkynyl, —O-cycloalkyl, —O-heterocyclyl, —O-aryl, —O-heteroaryl,—SO₂-cycloalkyl, —NR^{1L1}R^{1L2}, or C₂₋₆alkynyl-NR^{1L3}R^{1L4}, wherein each C₁₋₆-alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆-alkynyl, —O-cycloalkyl, —O-heterocyclyl, —O-aryl, —O-heteroaryl, or —SO₂-cycloalkyl of R^{1a4} is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂.

In some embodiments of the compounds of Formula I and stereoisomers and pharmaceutically acceptable salts thereof, one or more of R^{1a1}, R^{1a2}, R^{1a3}, and R^{1a4} is not H. In some embodiments, R^{1a1} is not H. In some embodiments, R^{1a2} is not H. In some embodiments, R^{1a3} is not H. In some embodiments, R^{1a4} is not H.

In some embodiments of the compounds of Formula I and stereoisomers and pharmaceutically acceptable salts thereof, one or more of R^{1a1}, R^{1a2}, R^{1a3}, and R^{1a4} is halo. In some embodiments, R^{1a1} is halo. In some embodiments, R^{1a2} is halo. In some embodiments, R^{1a3} is halo. In some embodiments, R^{1a4} is halo. In some embodiments, the halo is F, Cl, or Br. In some embodiments, the halo is Cl. In some embodiments, the halo is F. In some embodiments, R^{1a1} is F. In some embodiments, R^{1a3} is F. In some embodiments, R^{1a3} is F.

In some embodiments of the compounds of Formula I and stereoisomers and pharmaceutically acceptable salts thereof, one or more of R^{1a1}, R^{1a2}, R^{1a3}, and R^{1a4} is —OH. In some embodiments, R^{1a1} is —OH. In some embodiments, R^{1a2} is —OH. In some embodiments, R^{1a3} is —OH. In some embodiments, R^{1a4} is —OH.

In some embodiments of the compounds of Formula I and stereoisomers and pharmaceutically acceptable salts thereof, one or more of R^{1a1}, R^{1a2}, R^{1a3}, and R^{1a4} is C alkyl. In some embodiments, R^{1a1} is C₁₋₆alkyl. In some embodiments, R^{1a2} is C₁₋₆alkyl. In some embodiments, R^{1a3} is C₁₋₆alkyl. In some embodiments, R^{1a4} is C₁₋₆alkyl. In some embodiments, the C₁₋₆alkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the C₁₋₆alkyl is substituted with halo. In some embodiments, R^{1a1}, R^{1a2}, R^{1a3}, or R^{1a4} is —CH₃, —CH₂CH₃, —CH₂CF₃, —CF₃, —CF₂CH₃, —CH—CH₂CF₃,

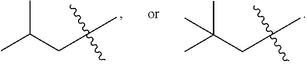

In some embodiments of the compounds of Formula I and stereoisomers and pharmaceutically acceptable salts thereof, one or more of R^{1a1}, R^{1a2}, R^{1a3}, and R^{1a4} is C₂₋₆alkenyl. In some embodiments, R^{1a1} is C₂₋₆alkenyl. In some embodiments, R^{1a2} is C₂₋₆alkenyl. In some embodiments, R^{1a3} is C₂₋₆alkenyl. In some embodiments, R^{1a4} is C₂₋₆alkenyl. In some embodiments, the C₂₋₆alkenyl is unsubstituted or substituted with one or more substituents independently selected from the group consisting of halo, —OH. C₁₋₆alkyl, C₁₋₆haloalkyl, cycloalkyl, halocycloalkyl, —O—C₁₋₆alkyl, and —C(O)NH₂. In some embodiments, the C₂₋₆alkenyl is substituted with halo. In some embodiments, R^{1a1}, R^{1a2}, R^{1a3}, or R^{1a4} is

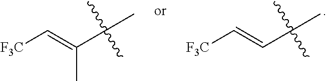

In some embodiments of the compounds of Formula I and stereoisomers and pharmaceutically acceptable salts thereof, one or more of R^{1a1}, R^{1a2}, R^{1a3}, and R^{1a4} is C₂₋₆alkynyl. In some embodiments, R^{1a1} is C₂₋₆alkynyl. In some embodiments, R^{1a2} is C₂₋₆alkynyl. In some embodiments, R^{1a3} is C₂₋₆alkynyl. In some embodiments, R^{1a4} is C₂₋₆alkynyl. In some embodiments, the C₂₋₆alkynyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the C₂₋₆alkynyl is substituted with halo. In some embodiments, the C₂₋₆alkynyl is substituted with cycloalkyl. In some embodiments, R^{1a1}, R^{1a2}, R^{1a3}, or R^{1a4} is

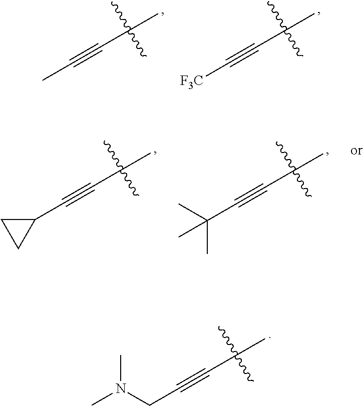

In some embodiments of the compounds of Formula I and stereoisomers and pharmaceutically acceptable salts thereof, one or more of R^{1a1}, R^{1a2}, R^{1a3}, and R^{1a4} is heterocyclyl. In some embodiments, R^{1a1} is heterocyclyl. In some embodiments, R^{1a2} is heterocyclyl. In some embodiments, R^{1a3} is heterocyclyl. In some embodiments, R^{1a4} is heterocyclyl. In some embodiments, the heterocyclyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the heterocyclyl is substituted with halo. In some embodiments, the heterocyclyl is substituted with —OH. In some embodiments, R^{1a1}, R^{1a2}, R^{1a3}, or R^{1a4} is

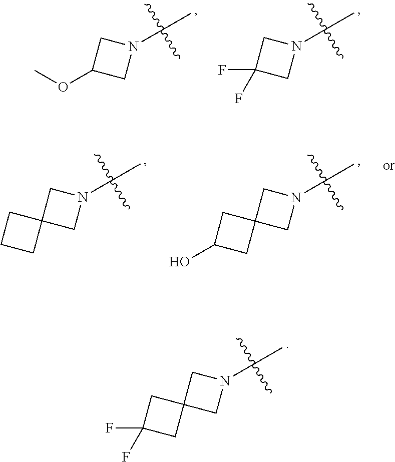

In some embodiments of the compounds of Formula I and stereoisomers and pharmaceutically acceptable salts thereof, one or more of R^{1a1}, R^{1a2}, R^{1a3}, and R^{1a4} is aryl or heteroaryl. In some embodiments, R^{1a1} is aryl or heteroaryl. In some embodiments, R^{1a2} is aryl or heteroaryl. In some embodiments, R^{1a3} is aryl or heteroaryl. In some embodiments, R^{1a4} is aryl or heteroaryl. In some embodiments, the aryl or heteroaryl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, R^{1a1}, R^{1a2}, R^{1a3}, or R^{1a4} is

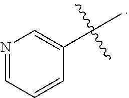 [SMILES: *c1cccnc1]

In some embodiments of the compounds of Formula I and stereoisomers and pharmaceutically acceptable salts thereof, one or more of R^{1a1}, R^{1a2}, R^{1a3}, and R^{1a4} is —O—C₁₋₆alkyl. In some embodiments, R^{1a1} is —O—C₁₋₆alkyl. In some embodiments, R^{1a2} is —O—C₁₋₆alkyl. In some embodiments, R^{1a3} is —O—C₁₋₆alkyl. In some embodiments, R^{1a4} is —O—C₁₋₆alkyl. In some embodiments, the —O—C₁₋₆alkyl is unsubstituted or substituted with one or more substituents independently selected from the group consisting of halo, —OH. C₁₋₆alkyl, C₁₋₆haloalkyl, cycloalkyl, halocycloalkyl, —O—C₁₋₆alkyl, and —C(O)NH₂. In some embodiments, the —O—C₁₋₆alkyl is substituted with halo. In some embodiments, the —O—C₁₋₆alkyl is substituted with cycloalkyl. In some embodiments, R^{1a1}, R^{1a2}, R^{1a3}, or R^{1a4} is —OCH₃, —OCHF₂, —OCH₂CF₃, or

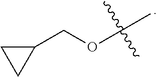 [SMILES: *OCC1CC1]

In some embodiments of the compounds of Formula I and stereoisomers and pharmaceutically acceptable salts thereof, one or more of R^{1a1}, R^{1a2}, R^{1a3}, and R^{1a4} is —O—C₂₋₆alkenyl. In some embodiments, R^{1a1} is —O—C₂₋₆alkenyl. In some embodiments, R^{1a2} is —O—C₂₋₆alkenyl. In some embodiments, R^{1a3} is —O—C₂₋₆alkenyl. In some embodiments, R^{1a4} is —O—C₂₋₆alkenyl. In some embodiments, the —O—C₂₋₆alkenyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O—C₂₋₆alkenyl is substituted with halo.

In some embodiments of the compounds of Formula I and stereoisomers and pharmaceutically acceptable salts thereof, one or more of R^{1a1}, R^{1a2}, R^{1a3}, and R^{1a4} is —C₂₋₆alkynyl. In some embodiments, R^{1a1} is —O—C₂₋₆alkynyl. In some embodiments, R^{1a2} is —O—C₂₋₆alkynyl. In some embodiments, R^{1a3} is —O—C₂₋₆ alkynyl. In some embodiments, R^{1a4} is —O—C₂₋₆alkynyl. In some embodiments, the —O—C₂₋₆alkynyl is unsubstituted or substituted with one or more substituents independently selected from the group consisting of halo, —OH, C₁₋₆alkyl, C₁₋₆haloalkyl, cycloalkyl, halocycloalkyl. O—C₁₋₆alkyl, and —C(O)NH₂. In some embodiments, the O—C₂₋₆alkynyl is substituted with halo. In some embodiments, R^{1a1}, R^{1a2}, R^{1a3}, or R^{1a4} is

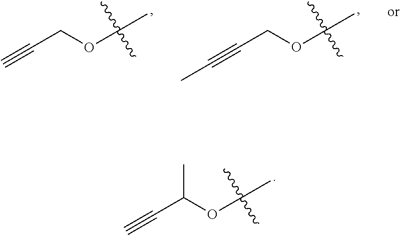

In some embodiments of the compounds of Formula I and stereoisomers and pharmaceutically acceptable salts thereof, one or more of R^{1a1}, R^{1a2}, R^{1a3}, and R^{1a4} is —O-cycloalkyl. In some embodiments, R^{1a1} is —O-cycloalkyl. In some embodiments, R^{1a2} is —O-cycloalkyl. In some embodiments, R^{1a3} is —O-cycloalkyl. In some embodiments, R^{1a4} is cycloalkyl. In some embodiments, the —O-cycloalkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-cycloalkyl is substituted with halo. In some embodiments, the —O-cycloalkyl is substituted with C₁₋₆haloalkyl. In some embodiments, R^{1a1}, R^{1a2}, R^{1a3}, or R^{1a4} is

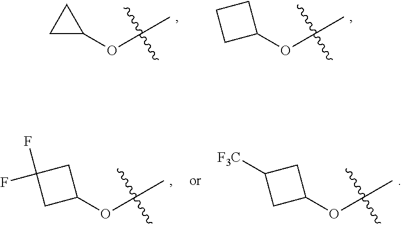 [SMILES: *OC1CC1 ; *OC1CCC1 ; *OC1CC(F)(F)C1 ; *OC1CC(C(F)(F)F)C1]

In some embodiments of the compounds of Formula I and stereoisomers and pharmaceutically acceptable salts thereof, one or more of R^{1a1}, R^{1a2}, R^{1a3}, and R^{1a4} is —O-heterocyclyl. In some embodiments, R^{1a1} is —O-heterocyclyl. In some embodiments, R^{1a2} is —O-heterocyclyl. In some embodiments, R^{1a3} is —O-heterocyclyl. In some embodiments, R^{1a4} is —O-heterocyclyl. In some embodiments, the —O-heterocyclyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-heterocyclyl is substituted with halo.

In some embodiments of the compounds of Formula I and stereoisomers and pharmaceutically acceptable salts thereof, one or more of R^{1a1}, R^{1a2}, R^{1a3}, and R^{1a4} is —O-aryl. In some embodiments, R^{1a1} is —O-aryl. In some embodiments, R^{1a2} is —O-aryl. In some embodiments, R^{1a3} is —O-aryl. In some embodiments, R^{1a4} is —O-aryl. In some embodiments, the —O-aryl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-aryl is substituted with halo. In some embodiments, the —O-aryl is substituted with C₁₋₆alkyl. In some embodiments, the —O-aryl is substituted with —C(O)NH₂. In some embodiments, the aryl of is a 6 membered monocyclic aryl. In some embodiments, R^{1a3} is —O-aryl wherein the aryl is an unsubstituted 6-membered monocyclic aryl. In some embodiments, R^{1a1}, R^{1a2}, R^{1a3}, or R^{1a4} is

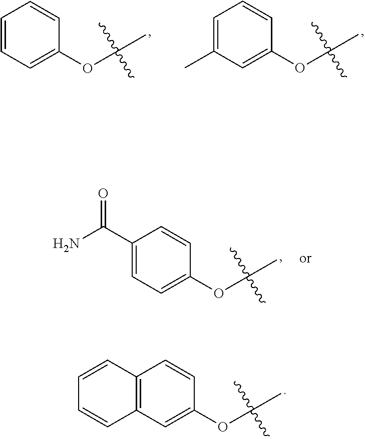

In some embodiments of the compounds of Formula I and stereoisomers and pharmaceutically acceptable salts thereof, one or more of R^{1a1}, R^{1a2}, R^{1a3}, and R^{1a4} is —O-heteroaryl. In some embodiments, R^{1a1} is —O-heteroaryl. In some embodiments, R^{1a2} is —O-heteroaryl. In some embodiments, R^{1a3} is —O-heteroaryl. In some embodiments, R^{1a4} is —O-heteroaryl. In some embodiments, the —O-heteroaryl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-heteroaryl is substituted with halo. In some embodiments, R^{1a1}, R^{1a2}, R^{1a3}, or R^{1a4} is

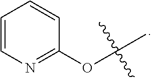 [SMILES: *Oc1ccccn1]

In some embodiments of the compounds of Formula I and stereoisomers and pharmaceutically acceptable salts thereof, one or more of R^{1a1}, R^{1a2}, R^{1a3}, and R^{1a4} is —SO-cycloalkyl. In some embodiments, R^{1a1} is—SO₂-cycloalkyl. In some embodiments, R^{1a2} is —SO₂-cycloalkyl. In some embodiments, R^{1a3} is —SO₂-cycloalkyl. In some embodiments, R^{1a4} is —SO₂-cycloalkyl. In some embodiments, the —SO₂-cycloalkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —SOZ-cycloalkyl is substituted with halo. In some embodiments, R^{1a1}, R^{1a2}, R^{1a3}, or R^{1a4} is

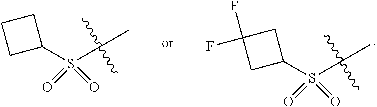 [SMILES: *S(=O)(=O)C1CCC1 ; *S(=O)(=O)C1CC(F)(F)C1]

In some embodiments of the compounds of Formula I and stereoisomers and pharmaceutically acceptable salts thereof, one or more of R^{1a1}, R^{1a2}, R^{1a3}, and R^{1a4} is —NR^{1L1}R^{1L2}. In some embodiments, R^{1a1} is —NR^{1L1}R^{1L2}. In some embodiments, R^{1a1} is —NR^{1L1}R^{1L2}. In some embodiments, R^{1a3} is —NR^{1L1}R^{1L2}. In some embodiments, R^{1a4} is —NR^{1L1}R^{1L2}. In some embodiments, R^{1a1}, R^{1a2}, R^{1a3}, or R^{1a4} is

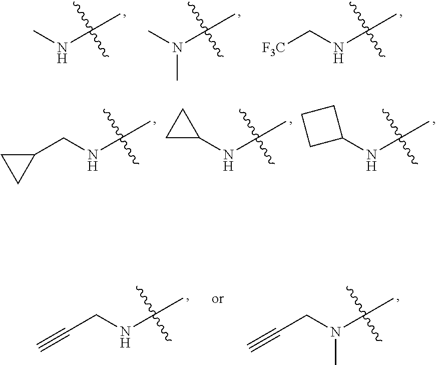

In some embodiments of the compounds of Formula I and stereoisomers and pharmaceutically acceptable salts thereof, R^{1L1} and R^{1L2} are independently H, C₁₋₆alkyl, C₂₋₆alkynyl, or cycloalkyl. In some embodiments, R^{1L1} is H. In some embodiments, R^{1L1} is H and R^{1L2} is C₁₋₆alkyl, C₂₋₆-alkynyl, or cycloalkyl. In some embodiments, R^{1L1} is —CH₃. In some embodiments, R^{1L1} is —CH₃ and R^{1L2} is C₁₋₆alkyl, C₂₋₆alkynyl, or cycloalkyl. In some embodiments, R^{1L1} is H or —CH₃ and R^{1L2} is C₁₋₆alkyl. In some embodiments, R^{1L1} is H or —CH₃ and R^{1L2} is C₂₋₆-alkynyl. In some embodiments, R^{1L1} is H or —CH₃ and R^{1L2} is cycloalkyl. In some embodiments, the C₁₋₆alkyl, C₂₋₆alkynyl, or cycloalkyl is unsubstituted or substituted with one or more substituents independently selected from the group consisting of halo, C₁₋₆alkyl, C₁₋₆haloalkyl, cycloalkyl, and halocycloalkyl. In some embodiments, the C₁₋₆alkyl is substituted with halo. In some embodiments, the C₁₋₆alkyl is substituted with cycloalkyl.

In some embodiments of the compounds of Formula I and stereoisomers and pharmaceutically acceptable salts thereof, one or more of R^{1a1}, R^{1a2}, R^{1a3}, and R^{1a4} is C₂₋₆-alkynyl-NR^{1L3}R^{1L4}. In some embodiments, R^{1a1} is C₂₋₆alkynyl-NR^{1L3}R^{1L4}. In some embodiments, R^{1a2} is C₂₋₆alkynyl-NR^{1L3}R^{1L4}. In some embodiments, R^{1a3} is C₂₋₆alkynyl-NR^{1L3}R^{1L4}. In some embodiments, R^{1a4} is C₂₋₆alkynyl-NR^{1L3}R^{1L4}.

In some embodiments of the compounds of Formula I and stereoisomers and pharmaceutically acceptable salts thereof, R^{1L3} and R^{1L4} are independently H, C₁₋₆alkyl, C₂₋₆alkynyl, or cycloalkyl. In some embodiments, R^{1L3} is H. In some embodiments, R^{1L3} is H and R^{1L4} is C₁₋₆alkyl, C₂₋₆-alkynyl, or cycloalkyl, In some embodiments, R^{1L3} is —CH₃. In some embodiments, R^{1L3} is —CH₃ and R^{1L4} is C₁₋₆alkyl, C₂₋₆alkynyl, or cycloalkyl. In some embodiments, R^{1L3} is C₁₋₆alkyl and R^{1L4} is C₁₋₆alkyl. In some embodiments, R^{1L3} is H or —CH₃ and R^{1L4} is C₁₋₆alkyl. In some embodiments, R^{1L3} is H or —CH₃ and R^{1L4} is C₂₋₆alkynyl. In some embodiments, R^{1L3} is H or —CH₃ and R^{1L4} is cycloalkyl. In some embodiments, the C₁₋₆alkyl, C₂₋₆alkynyl, or cycloalkyl is unsubstituted or substituted with one or more substituents independently selected from the group consisting of halo, C₁₋₆alkyl, C₁₋₆haloalkyl, cycloalkyl, and halocycloalkyl. In some embodiments, the C₁₋₆alkyl is substituted with halo. In some embodiments, the C₁₋₆alkyl is substituted with cycloalkyl. In some embodiments, R^{1a1}, R^{1a2}, R^{1a3}, or R^{1a4} is

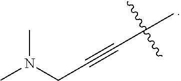 [SMILES: *C#CCN(C)C]

In some embodiments of the compounds of Formula I and stereoisomers and pharmaceutically acceptable salts thereof, two or more of R^{1a1}, R^{1a2}, R^{1a3}, and R^{1a4} is not H. In some embodiments, R^{1a3} is not H and one of R^{1a1}, R^{1a2}, or R^{1a4} is not H. In some embodiments, R^{1a2} is F or Cl and R^{1a3} is —O—C₁₋₆alkyl or —O-cycloalkyl, wherein the —O—C₁₋₆alkyl or —O-cycloalkyl is unsubstituted or substituted with halo or —CN. In some embodiments, R^{1a1} is not H and R^{1a2} is not H. In some embodiments, R^{1a1} is F or Cl and R^{1a2} is F or Cl.

In some embodiments of the compounds of Formula I and stereoisomers and pharmaceutically acceptable salts thereof, X³ is —O—. In some embodiments, X³ is —NR^{1b1}—. In some embodiments, X³ is —NH—. In some embodiments, X³ is —N(CH₃)—. In some embodiments, X³ is —CR^{1b2}R^{1b3}. In some embodiments, X³ is —CH(CH₃)—. In some embodiments, X³ is —CD₂-. In some embodiments, X³ is —CH₂—.

In some embodiments of the compounds of Formula I and stereoisomers and pharmaceutically acceptable salts thereof. X⁴ is —O—. In some embodiments, X⁴ is —NR^{3f2}—. In some embodiments, X⁴ is —NH—. In some embodiments, X⁴ is —N(CH₃)—. In some embodiments, X⁴ is —CR^{3f3}R^{3f4}—. In some embodiments, X⁴ is —CH(CH₃)—. In some embodiments, X⁴ is —CH₂—.

In some embodiments of the compounds of Formula I and stereoisomers and pharmaceutically acceptable salts thereof. A1 is

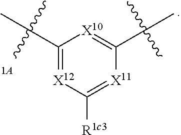

In some embodiments, one or more of X¹⁰, X¹¹, and X¹² is —N—. In some embodiments, X¹⁰ is —N—. In some embodiments, X¹¹ is —N—. In some embodiments, X¹² is —N—. In some embodiments, X¹⁰ is —CR^{1c1}—. In some embodiments, X¹¹ is —CR^{1c2}—. In some embodiments, X¹² is —CR^{1c3}. In some embodiments, X¹⁰ is —CR^{1c1}, X¹¹ is —CR^{1c2}—, and X¹² is —CR^{1c3}—.

In some embodiments of the compounds of Formula I and stereoisomers and pharmaceutically acceptable salts thereof, one or more of R^{1c1}, R^{1c2}, R^{1c3}, and R^{1c4} is not H. In some embodiments, one or more of R^{1c1}, R^{1c2}, R^{1c3}, and R^{1c4} is —CN. In some embodiments, one or more of R^{1c1}, R^{1c2}, R^{1c3}, and R^{1c4} is independently Cl, F, or Br. In some embodiments, two or more of R^{1c1}, R^{1c2}, R^{1c3}, and R^{1c4} are independently Cl, F, or Br. In some embodiments, R^{1c2} is F and R^{1c1}, R^{1c3}, and R^{1c4} are H. In some embodiments, R^{1c1}, R^{1c2}, R^{1c3}, and R^{1c4} are H.

In some embodiments of the compounds of Formula I and stereoisomers and pharmaceutically acceptable salts thereof, A1 is

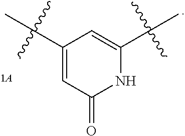 [SMILES: "." ; *c1cc(*)[nH]c(=O)c1]

In some embodiments, A1 is

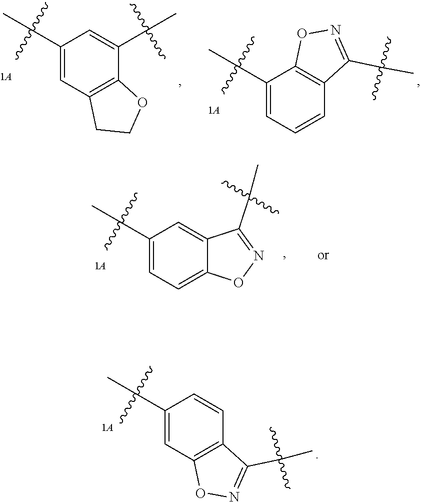 [SMILES: *c1cc(*)c2c(c1)CCO2 ; *c1noc2c(*)cccc12 ; *c1ccc2onc(*)c2c1 ; *c1ccc2c(*)noc2c1]

In some embodiments, A1 is

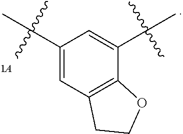 [SMILES: *c1cc(*)c2c(c1)CCO2]

In some embodiments, A1 is

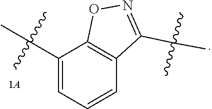 [SMILES: *c1noc2c(*)cccc12]

In some embodiments, A1 is

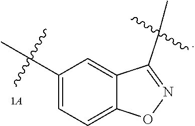 [SMILES: *c1ccc2onc(*)c2c1]

In some embodiments, A1 is

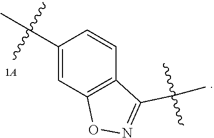 [SMILES: *c1ccc2c(*)noc2c1]

In some embodiments of the compounds of Formula I and stereoisomers and pharmaceutically acceptable salts thereof, B3, B4, B5, or B6 is a 3-membered monocyclic heterocyclediyl, a 4-membered monocyclic heterocyclediyl, a 5-membered monocyclic heterocyclediyl comprising 2 or more N, a 6-membered monocyclic heterocyclediyl comprising 2 or more N, a 7-membered monocyclic heterocyclediyl, an 8-membered monocyclic heterocyclediyl, a 7 to 18-membered polycyclic heterocyclediyl, or a 7 to 18-membered spirocyclic heterocyclediyl, wherein the 3-membered monocyclic heterocyclediyl, 4-membered monocyclic heterocyclediyl, 5-membered monocyclic heterocyclediyl, 6-membered monocyclic heterocyclediyl, 7-membered monocyclic heterocyclediyl, 8-membered monocyclic heterocyclediyl, 7 to 18-membered polycyclic heterocyclediyl, or 7 to 18-membered spirocyclic heterocyclediyl of B3, B4, B5, or B6 is unsubstituted or substituted with one or more substituents independently selected from the group consisting of halo, C₁₋₆alkyl, and oxo. In some embodiments, the 7 to 18-membered polycyclic heterocyclediyl is a 7, 8, 9, 10, 11, 12, 13, 14, 15, 16, 17, or 18-membered polycyclic heterocyclediyl. In some embodiments, the 7 to 18-membered spirocyclic heterocyclediyl is a 7, 8, 9, 10, 11, 12, 13, 14, 15, 16, 17, or 18-membered spirocyclic heterocyclediyl.

In some embodiments of the compounds of Formula I and stereoisomers and pharmaceutically acceptable salts thereof, B3, B4, B5, or B6 is a 3 to 8-membered monocyclic heterocyclediyl, wherein the 3 to 8-membered monocyclic heterocyclediyl is unsubstituted or substituted with one or more substituents selected from the group consisting of halo, C₁₋₆alkyl, and oxo. In some embodiments, the 3 to 8-membered monocyclic heterocyclediyl is a 3, 4, 5, 6, 7, or 8-membered monocyclic heterocyclediyl. In some embodiments, B3, B4, B5, or B6 is a 4-membered monocyclic heterocyclediyl, wherein the 4-membered monocyclic heterocyclediyl is unsubstituted or substituted with one or more substituents selected from the group consisting of halo, C₁₋₆alkyl, and oxo. In some embodiments, B3, B4, B5, or B6 is

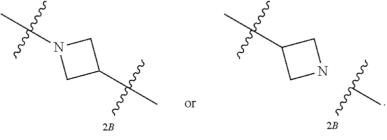

In some embodiments, B3, B4, B5, or B6 is a 6-membered monocyclic heterocyclediyl, wherein the 6-membered monocyclic heterocyclediyl is unsubstituted or substituted with one or more substituents selected from the u consisting of halo, C₁₋₆alkyl, and oxo. In some embodiments, B3, B4, B5, or B6 is

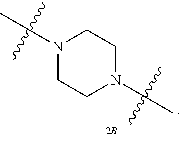 [SMILES: *N1CCN(*)CC1]

In some embodiments, B3, B4, B5, or B6 is a 7-membered monocyclic heterocyclediyl, wherein the 7-membered monocyclic heterocyclediyl is unsubstituted or substituted with one or more substituents selected from the group consisting of halo, C₁₋₆alkyl, and oxo. In some embodiments, B3, B4, B5, or B6 is

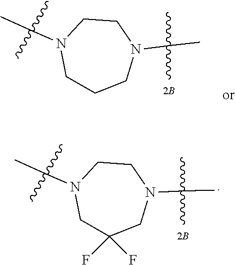 [SMILES: *N1CCCN(*)CC1 ; *N1CCN(*)CC(F)(F)C1]

In some embodiments of the compounds of Formula I and stereoisomers and pharmaceutically acceptable salts thereof, B3, B4, B5, or B6 is a 7 to 18-membered polycyclic heterocyclediyl, wherein the 7 to 18-membered polycyclic heterocyclediyl is unsubstituted or substituted with one or more substituents selected from the group consisting of halo, C₁₋₆alkyl, and oxo. In some embodiments, the 7 to 18-membered polycyclic heterocyclediyl is a 7, 8, 9, 10, 11, 12, 13, 14, 15, 16, 17, or 18-membered polycyclic heterocyclediyl. In some embodiments, B3, B4, B5, or B6 is

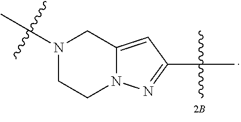 [SMILES: *c1cc2n(n1)CCN(*)C2]

In some embodiments, B3, B4, B5, or B6 is

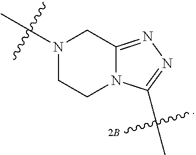 [SMILES: *c1nnc2n1CCN(*)C2]

In some embodiments B3, B4, B5, or B6 is

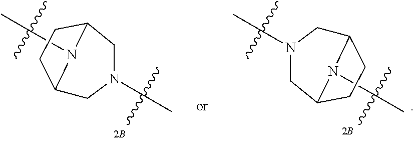 [SMILES: *N1CC2CCC(C1)N2* ; *N1CC2CCC(C1)N2*]

In some embodiments, B3, B4, B5, or B6 is

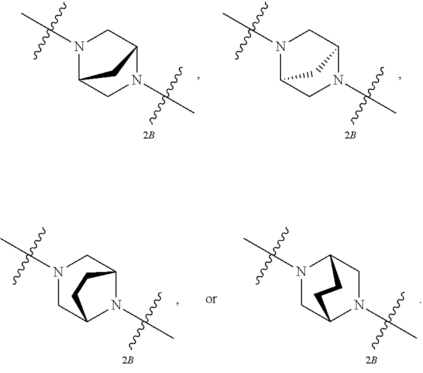

In some embodiments, B3, B4, B5, or B6 is

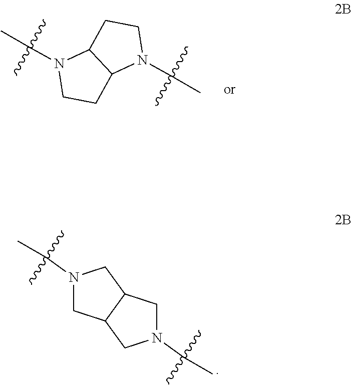 [SMILES: *N1CCC2C1CCN2* ; *N1CC2CN(*)CC2C1]

In some embodiments, B3, B4, B5, or B6 is

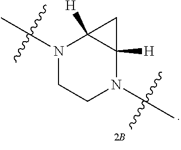 [SMILES: *N1CCN(*)[C@H]2C[C@H]21]

In some embodiments of the compounds of Formula I and stereoisomers and pharmaceutically acceptable salts thereof, B3, B4, B5, or B6 is a 7 to 18-membered spirocyclic heterocyclediyl, wherein the 7 to 18-membered spirocyclic heterocyclediyl is unsubstituted or substituted with one or more substituents selected from the group consisting of halo, C₁₋₆alkyl, and oxo. In some embodiments, the 7 to 18-membered spirocyclic heterocyclediyl is a 7, 8, 9, 10, 11, 12, 13, 14, 15, 16, 17, or 18-membered spirocyclic heterocyclediyl. In some embodiments, B3, B4, B5, or B6 is

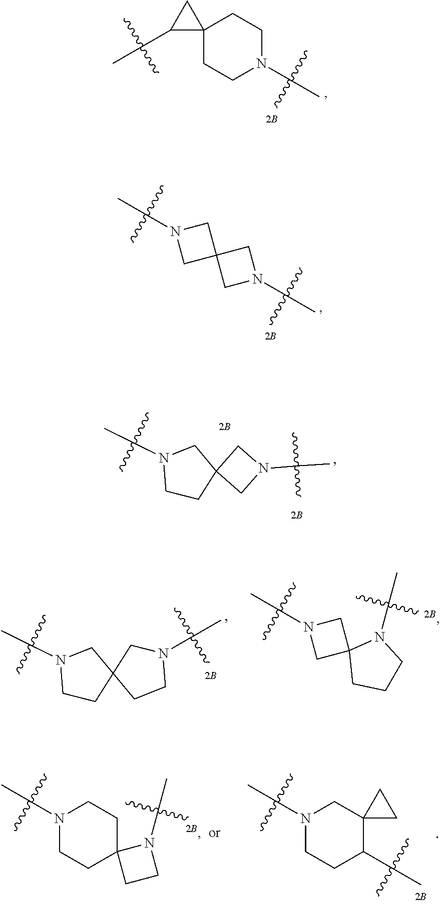

In some embodiments of the compounds of Formula I and stereoisomers and pharmaceutically acceptable salts thereof, the monocyclic heterocyclediyl, polycyclic heterocyclediyl, or spirocyclic heterocyclediyl of B3, B4, B5, or B6 comprises one or more N. In some embodiments, the monocyclic heterocyclediyl, polycyclic heterocyclediyl, or spirocyclic heterocyclediyl of B3, B4, B5, or B6 comprises two or more N.

In some embodiments of the compounds of Formula I and stereoisomers and pharmaceutically acceptable salts thereof, R^{5g1} is H. In some embodiments, R^{5g1} is C₁₋₆alkyl. In some embodiments, R^{5g1} is —CH₃.

In some embodiments of the compounds of Formula I and stereoisomers and pharmaceutically acceptable salts thereof, R^{7g2} is H. In some embodiments, R^{7g2} is C₁₋₆alkyl. In some embodiments, R^{7g2} is —CH₃. In some embodiments, R^{7g2} is —CH₂CH₃.

In some embodiments of the compounds of Formula I and stereoisomers and pharmaceutically acceptable salts thereof. X⁵ is —CH(R^{7g3})—, wherein R^{7g3} is H or C₁₋₆alkyl. In some embodiments, X⁵ is —CH₂—. In some embodiments, X is —CH(CH₃)—.

In some embodiments of the compounds of Formula I and stereoisomers and pharmaceutically acceptable salts thereof, X is —CH(R^{7g4})CH₂N(R^{7g5})—, wherein R^{7g4} and R^{7g5} are independently H or C₁₋₆alkyl. In some embodiments, R^{7g4} is H and R^{7g5} is C₁₋₆alkyl. In some embodiments, R^{7g5} is —CH₃. In some embodiments, R^{7g5} is —CH₂CH₃. In some embodiments, R^{7g4} is H and R^{7g5} is H.

In some embodiments of the compounds of Formula I and stereoisomers and pharmaceutically acceptable salts thereof, D3, D4, D5, D6, or D7 is C₁₋₆alkyl, wherein the C₁₋₆alkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{1D10})₂. In some embodiments, the C₁₋₆alkyl is unsubstituted or substituted with halo. In some embodiments, the C₁₋₆alkyl is unsubstituted or substituted with —OH. In some embodiments, the C₁₋₆alkyl is unsubstituted or substituted with —O—C₁₋₆alkyl.

In some embodiments of the compounds of Formula I and stereoisomers and pharmaceutically acceptable salts thereof, D3, D4, D5, D6, or D7 is cycloalkyl, wherein the cycloalkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{1D10})₂. In some embodiments, the cycloalkyl is unsubstituted or substituted with halo. In some embodiments, D3, D4, D5, D6, or D7 is

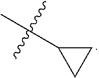 [SMILES: *C1CC1]

In some embodiments of the compounds of Formula I and stereoisomers and pharmaceutically acceptable salts thereof, D3, D4, D5, D6, or D7 is aryl, wherein the aryl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆ haloalkyl, —C(O)-cycloalkyl, and —C(O)N(R^{1D10})₂. In some embodiments, the aryl is a monocyclic 6-membered aryl. In some embodiments, the aryl is substituted with one or more substituents selected from the group consisting of halo, —CN, C₁₋₆alkyl, and C₁₋₆haloalkyl. In some embodiments, the aryl is substituted with —CN.

In some embodiments of the compounds of Formula I and stereoisomers and pharmaceutically acceptable salts thereof, D3, D4, D5, D6, or D7 is heteroaryl, wherein the heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, —C(O)NH₂, —C(O)NHCH₃, and —C(O)N(CH₃)₂.

In some embodiments of the compounds of Formula I and stereoisomers and pharmaceutically acceptable salts thereof, D3, D4, D5, D6, or D7 is a monocyclic 5 or 6-membered heteroaryl comprising one or more N, wherein the monocyclic 5 or 6-membered heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, —C(O)NH₂, —C(O)NHCH₃, and —C(O)N(CH₃)₂. In some embodiments, the monocyclic 5 or 6-membered heteroaryl is substituted with —CN. In some embodiments, the monocyclic 5 or 6-membered heteroaryl is substituted with —CF₃. In some embodiments, the monocyclic 5 or 6-membered heteroaryl is substituted with —OCH₃.

In some embodiments of the compounds of Formula I and stereoisomers and pharmaceutically acceptable salts thereof, D3, D4, D5, D6, or D7 is —O—C₁₋₆alkyl, wherein the —O—C₁₋₆alkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{1D10})₂. In some embodiments, the —O—C₁₋₆alkyl is unsubstituted or substituted with halo.

In some embodiments of the compounds of Formula I and stereoisomers and pharmaceutically acceptable salts thereof, D3, D4, D5, D6, or D7 is —O-aryl or —O-heteroaryl, wherein the —O-aryl or —O-heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{1D10})₂. In some embodiments, the —O-aryl or —O-heteroaryl is unsubstituted or substituted with halo. In some embodiments, the —O-aryl or —O-heteroaryl is unsubstituted or substituted with —CN. In some embodiments, D3, D4, D5, D6, or D7 is

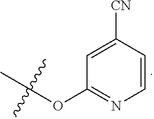 [SMILES: *Oc1cc(C#N)ccn1]

In some embodiments of the compounds of Formula I and stereoisomers and pharmaceutically acceptable salts thereof, D3, D4, D5, D6, or D7 is —C(O)—C₁₋₆alkyl, wherein the —C(O)—C₁₋₆alkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{1D10})₂. In some embodiments, the —C(O)—C₁₋₆alkyl is unsubstituted or substituted with halo. In some embodiments, the —C(O)—C₁₋₆alkyl is unsubstituted or substituted with —CN. In some embodiments, D3, D4, D5, D6, or D7 is

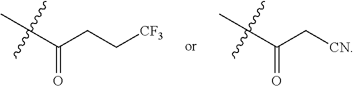 [SMILES: *C(=O)CCC(F)(F)F ; "or" ; *C(=O)CC#N]

In some embodiments of the compounds of Formula I and stereoisomers and pharmaceutically acceptable salts thereof, D3, D4, D5, D6, or D7 is —C(O)-cycloalkyl, wherein the —C(O)-cycloalkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{1D10})₂. In some embodiments, the —C(O)-cycloalkyl is unsubstituted or substituted with halo. In some embodiments, D3, D4, D5, D6, or D7 is

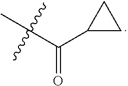 [SMILES: *C(=O)C1CC1]

In some embodiments of the compounds of Formula I and stereoisomers and pharmaceutically acceptable salts thereof, D3, D4, D5, D6, or D7 is —C(O)-heterocyclyl, wherein the —C(O)-heterocyclyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{1D10})₂. In some embodiments, the —C(O)-heterocyclyl is unsubstituted or substituted with halo. In some embodiments, D3, D4, D5, D6, or D7 is

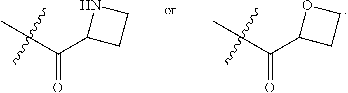 [SMILES: *C(=O)C1CCN1 ; *C(=O)C1CCO1]

In some embodiments of the compounds of Formula I and stereoisomers and pharmaceutically acceptable salts thereof, D3, D4, D5, D6, or D7 is —C(O)-aryl or —C(O)-heteroaryl, wherein the —C(O)-aryl or —C(O)-heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{1D10})₂. In some embodiments, the —C(O)-aryl or —C(O)-heteroaryl is unsubstituted or substituted with halo. In some embodiments, the —C(O)-aryl or —C(O)-heteroaryl is unsubstituted or substituted with —CF₃. In some embodiments, the —C(O)-aryl or —C(O)-heteroaryl is unsubstituted or substituted with —CN. In some embodiments, D3, D4, D5, D6, or D7 is

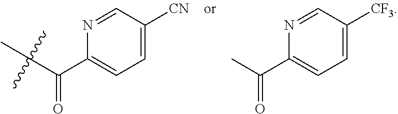

In some embodiments of the compounds of Formula I and stereoisomers and pharmaceutically acceptable salts thereof, D3, D4, D5, D6, or D7 is —N(R^{1D1})(R^{1D2}). In some embodiments, D3, D4, D5, D6 or D7 is

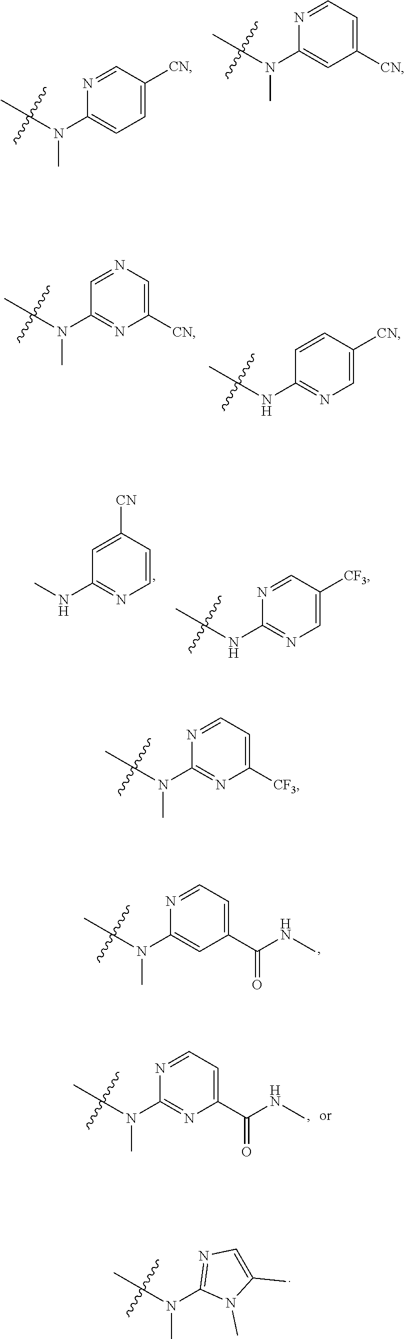

In some embodiments of the compounds of Formula I and stereoisomers and pharmaceutically acceptable salts thereof, R^{1D1} is H. In some embodiments, R^{1D1} is C₁₋₆alkyl. In some embodiments, R^{1D2} is aryl. In some embodiments, R^{1D2} is heteroaryl. In some embodiments, the heteroaryl is a 6-membered heteroaryl.

In some embodiments of the compounds of Formula I and stereoisomers and pharmaceutically acceptable salts thereof, D3, D4, D5, D6, or D7 is —C(O)N(R^{1D3})(R^{1D4}). In some embodiments, D3, D4, D5, D6, or D7 is

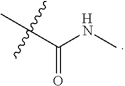

In some embodiments of the compounds of Formula I and stereoisomers and pharmaceutically acceptable salts thereof, R^{1D3} is H. In some embodiments, R^{1D3} is C₁₋₆alkyl. In some embodiments, R^{1D4} is C₁₋₆alkyl. In some embodiments, R^{1D3} is H and R^{1D4} is C₁₋₆alkyl.

In some embodiments of the compounds of Formula I and stereoisomers and pharmaceutically acceptable salts thereof, D3, D4, D5, D6, or D7 is —N(R^{1D5})C(O)R^{1D6}. In some embodiments, D3, D4, D5, D6, or D7 is

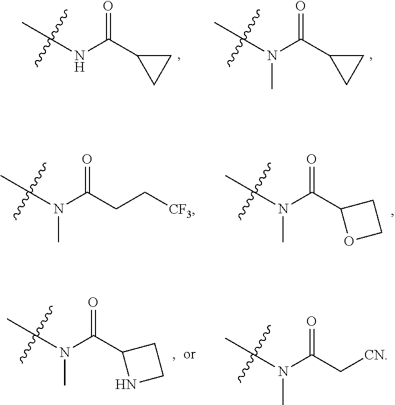

In some embodiments of the compounds of Formula I and stereoisomers and pharmaceutically acceptable salts thereof, R^{1D5} is H. In some embodiments, R^{1D5} is C₁₋₆alkyl. In some embodiments, R^{1D6} is C₁₋₆alkyl. In some embodiments, R^{1D6} is cycloalkyl. In some embodiments, R^{1D6} is heterocyclyl. In some embodiments, R^{1D5} is H and R^{1D6} is C₁₋₆alkyl, cycloalkyl, or heterocyclyl.

In some embodiments, the compound is of Formula II:

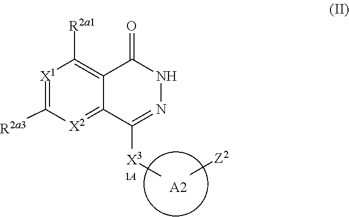

and stereoisomers and pharmaceutically acceptable salts thereof, wherein:
X¹ is —N— or —CR^{2a2}—;
X² is —N— or —CR^{2a4}—;
R^{2a1}, R^{2a2}, R^{2a3}, and R^{2a4} are independently H, halo, —OH, C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, —SO₂-cycloalkyl, —NR^{2L1}R^{2L2}, or C₂₋₆alkynyl-NR^{2L3}R^{2L4};
wherein if X¹ is —CR^{2a2}— and X² is —CR^{2a4}—, then at least one of R^{2a1}, R^{2a2}, R^{2a3}, and R^{2a4} is not H;
wherein each C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, or —SO₂-cycloalkyl of R^{2a1}, R^{2a2}, R^{2a3}, and R^{2a4} is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂;
R^{2L1}, R^{2L2}, R^{2L3}, and R^{2L4} are independently H, C₁₋₆alkyl, C₂₋₆alkynyl, or cycloalkyl, wherein each C₁₋₆alkyl, C₂₋₆alkynyl, or cycloalkyl of R^{2L1}, R^{2L2}, R^{2L3}, and R^{2L4} is unsubstituted or substituted with one or more substituents independently selected from the group consisting of halo, C₁₋₆alkyl, C₁₋₆haloalkyl, cycloalkyl, and halocycloalkyl;
X³ is —O—. —NR^{2b1}—, or —CR^{2b2}R^{2b3}, wherein R^{2b1}, R^{2b2}, and R^{2b3} are independently H or C₁₋₆alkyl;
A2 is

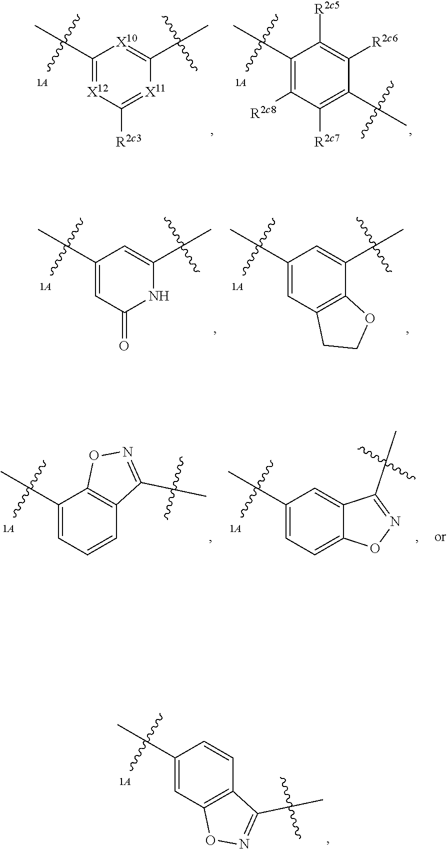

wherein a bond marked 1A is to X³;
X¹⁰ is —N— or —CR^{2c1}—, X¹¹ is —N— or —CR^{2c2}—, and X¹² is —N— or —CR^{2c4}—;
R^{2c1}, R^{2c2}, R^{2c3}, R^{2c4}, R^{2c5}, R^{2c6}, R^{2c7}, and R^{2c8} are independently H, halo, —CN, —SO₂CH₃, —SO₂NH₂, or —NHSO₂CH₃;
Z² is

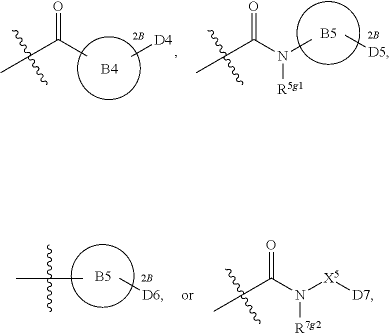

wherein a bond marked 2B is to D4, D5, or D6;
X⁵ is —CH(R^{7g3})— or —CH(R^{7g4})CH₂N(R^{7g5})—;
R^{5g1}, R^{7g2}, R^{7g3}, R^{7g4}, and R^{7g5} are independently H or C₁₋₆alkyl;
B4, B5, and B6 are independently a 3 to 8-membered monocyclic heterocyclediyl, a 7 to 18-membered polycyclic heterocyclediyl, or a 7 to 18-membered spirocyclic heterocyclediyl;
wherein the 3 to 8-membered monocyclic heterocyclediyl, 7 to 18-membered polycyclic heterocyclediyl, or 7 to 18-membered spirocyclic heterocyclediyl of B4, B5, and B6 is unsubstituted or substituted with one or more substituents independently selected from the group consisting of halo, C₁₋₆alkyl, and oxo;
provided that B4 is not

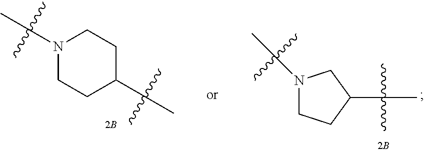 [SMILES: *C1CCN(*)CC1 ; *C1CCN(*)C1]

D4, D5, D6, and D7 are independently C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, —C(O)-heteroaryl, —N(R^{2D1})(R^{2D2}), —C(O)N(R^{2D3})(R^{2D4}), or —N(R^{2D5})C(O)R^{2D6};
wherein the C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, or —C(O)-heteroaryl of D4, D5, D6, and D7 is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{2D10})₂, wherein each R^{2D10} is independently H or C₁₋₆alkyl;
R^{2D1}, R^{2D3}, and R^{2D5} are independently H or C₁₋₆alkyl;
R^{2D2} is aryl or heteroaryl, wherein the aryl or heteroaryl of R^{2D2} is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{2D11})₂, wherein each R^{2D11} is independently H or C₁₋₆alkyl; and
R^{2D4} and R^{2D6} are independently C₁₋₆alkyl, cycloalkyl, heterocyclyl, aryl, or heteroaryl, wherein each C₃₋₆alkyl, cycloalkyl, heterocyclyl, aryl, or heteroaryl of R^{2D4} and R^{2D6} is unsubstituted or substituted with one or more substituents independently selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆ haloalkyl, —C(O)-cycloalkyl, and —C(O)N(R^{2D12})₂, wherein each R^{2D12} is independently H or C₁₋₆alkyl;
provided that if B4 is

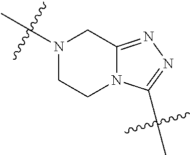 [SMILES: *c1nnc2n1CCN(*)C2]

and D4 is —CHF₂, —CF₂CH₃, or —CF₂CF₃, then R^{2a2} is not F, if X³ is —O—, B4 is

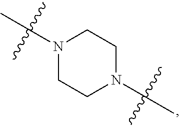 [SMILES: *N1CCN(*)CC1]

and D4 is —C(O)-aryl, then R^{2a4} is not Cl, and if D4 is

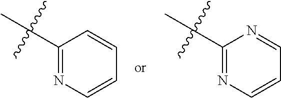 [SMILES: *c1ccccn1 ; *c1ncccn1]

and R^{2c2} is F, then R^{2a2} is not F.

In some embodiments of the compounds of Formula II and stereoisomers and pharmaceutically acceptable salts thereof, X¹ is —CR^{2a2}—. In some embodiments, X² is —CR^{2a4}—. In some embodiments, X¹ is —N—. In some embodiments, X² is —N—. In some embodiments, X¹ is —CR^{2a2}— and X² is —CR^{2a4}—. In some embodiments. X¹ is —CR^{2a2}— and X² is —N—. In some embodiments, X¹ is —N— and X² is —CR^{2a4}—.

In some embodiments of the compounds of Formula II and stereoisomers and pharmaceutically acceptable salts thereof, R^{2a2} is H, Cl, Br, I, —OH, C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, —SO₂-cycloalkyl, —NR^{2L1}R^{2L2}, or C₂₋₆alkynyl-NR^{2L3}R^{2L4}, wherein each C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, or —SO₂-cycloalkyl of R^{2a2} is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂.

In some embodiments of the compounds of Formula II and stereoisomers and pharmaceutically acceptable salts thereof, R^{2a4} is H, F, Br, I, —OH, C₁₋₆ alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, —SO₂-cycloalkyl, —NR^{2L1}R^{2L2}, or C₁₋₆alkynyl-NR^{2L3}R^{2L4}, wherein each C₁₋₆alkyl, C₁₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, or —SO-cycloalkyl of R^{2a4} is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂.

In some embodiments of the compounds of Formula II and stereoisomers and pharmaceutically acceptable salts thereof, one or more of R^{2a1}, R^{2a2}, R^{2a3}, and R^{2a4} is not H. In some embodiments, R^{2a1} is not H. In some embodiments, R^{2a2} is not H. In some embodiments, R^{2a3} is not H. In some embodiments, R^{2a4} is not H.

In some embodiments of the compounds of Formula II and stereoisomers and pharmaceutically acceptable salts thereof, one or more of R^{2a1}, R^{2a2}, R^{2a3}, and R^{2a4} is halo. In some embodiments, R^{2a1} is halo. In some embodiments, R^{2a2} is halo. In some embodiments, R^{2a3} is halo. In some embodiments, R^{2a4} is halo. In some embodiments, the halo is F, Cl, or Br. In some embodiments, the halo is Cl. In some embodiments, the halo is F. In some embodiments, R^{2a1} is F. In some embodiments, R^{2a3} is F. In some embodiments, R^{2a3} is F.

In some embodiments of the compounds of Formula II and stereoisomers and pharmaceutically acceptable salts thereof, one or more of R^{2a1}, R^{2a2}, R^{2a3}, and R^{2a4} is —OH. In some embodiments, R^{2a1} is —OH. In some embodiments, R^{2a2} is —OH. In some embodiments, R^{2a3} is —OH. In some embodiments, R^{2a4} is —OH.

In some embodiments of the compounds of Formula II and stereoisomers and pharmaceutically acceptable salts thereof, one or more of R^{2a1}, R^{2a2}, R^{2a3}, and R^{2a4} is C₁₋₆alkyl. In some embodiments, R^{2a1} is C₁₋₆alkyl. In some embodiments, R^{2a2} is C₁₋₆alkyl. In some embodiments, R^{2a3} is C₁₋₆alkyl. In some embodiments, R^{2a4} is C₁₋₆alkyl. In some embodiments, the C₁₋₆alkyl is unsubstituted or substituted with one or more substituents independently selected from the group consisting of halo, —OH, C₁₋₆alkyl, C₁₋₆ haloalkyl, cycloalkyl, halocycloalkyl, —O—C₁₋₆alkyl, and —C(O)NH₂. In some embodiments, the C₁₋₆alkyl is substituted with halo. In some embodiments, R^{2a1}, R^{2a2}, R^{2a3}, or R^{2a4} is —CH₃, —CH₂CH₃, —CH₂CF₃, —CF₃, —CF₂CH₃, —CH₂CH₂CF₃,

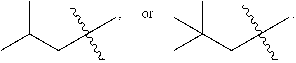

In some embodiments of the compounds of Formula II and stereoisomers and pharmaceutically acceptable salts thereof, one or more of R^{2a1}, R^{2a2}, R^{2a3}, and R^{2a4} is C₂₋₆alkenyl. In some embodiments, R^{2a1} is C₂₋₆alkenyl. In some embodiments, R^{2a2} is C₂₋₆alkenyl. In some embodiments, R^{2a3} is C₁₋₆alkenyl. In some embodiments, R^{2a4} is C₂₋₆alkenyl. In some embodiments, the C₂₋₆alkenyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the C₂₋₆alkenyl is substituted with halo. In some embodiments, R^{2a1}, R^{2a2}, R^{2a3}, or R^{2a4} is

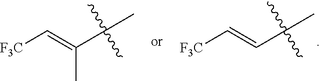

In some embodiments of the compounds of Formula II and stereoisomers and pharmaceutically acceptable salts thereof, one or more of R^{2a1}, R^{2a2}, R^{2a3}, and R^{2a4} is C₂₋₆alkynyl. In some embodiments, R^{2a1} is C₂₋₆alkynyl. In some embodiments, R^{2a2} is C₂₋₆alkynyl. In some embodiments, R^{2a3} is C₂₋₆alkynyl. In some embodiments, R^{2a4} is C₂₋₆alkynyl. In some embodiments, the C₂₋₆alkynyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the C₁₋₆alkynyl is substituted with halo. In some embodiments, the C₁₋₆alkynyl is substituted with cycloalkyl. In some embodiments, R^{2a1}, R^{2a2}, R^{2a3}, or R^{2a4} is

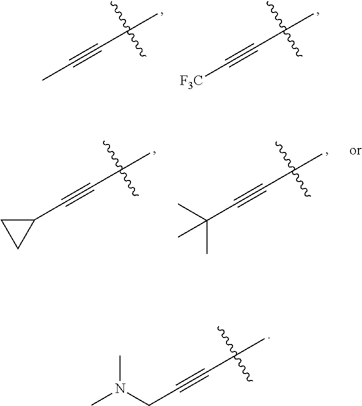

In some embodiments of the compounds of Formula II and stereoisomers and pharmaceutically acceptable salts thereof, one or more of R^{2a1}, R^{2a2}, R^{2a3}, and R^{2a4} is cycloalkyl. In some embodiments, R^{2a1} is cycloalkyl. In some embodiments, R^{2a2} is cycloalkyl. In some embodiments, R^{2a3} is cycloalkyl. In some embodiments, R^{2a4} is cycloalkyl. In some embodiments, the cycloalkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the cycloalkyl is substituted with halo. In some embodiments, R^{2a1}, R^{2a2}, R^{2a3}, or R^{2a4} is

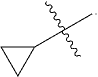 [SMILES: *C1CC1]

In some embodiments of the compounds of Formula II and stereoisomers and pharmaceutically acceptable salts thereof, one or more of R^{2a1}, R^{2a2}, R^{2a3}, and R^{2a4} is heterocyclyl. In some embodiments, R^{2a1} is heterocyclyl. In some embodiments, R^{2a2} is heterocyclyl. In some embodiments, R^{2a3} is heterocyclyl. In some embodiments, R^{2a4} is heterocyclyl. In some embodiments, the heterocyclyl is unsubstituted or substituted with one or more substituents independently selected from the group consisting of halo, —OH, C₁₋₆alkyl, C₁₋₆-haloalkyl, cycloalkyl, halocycloalkyl, —O—C₁₋₆alkyl, and —C(O)NH₂. In some embodiments, the heterocyclyl is substituted with halo. In some embodiments, the heterocyclyl is substituted with —OH. In some embodiments, R^{2a1}, R^{2a2}, R^{2a3}, or R^{2a4} is

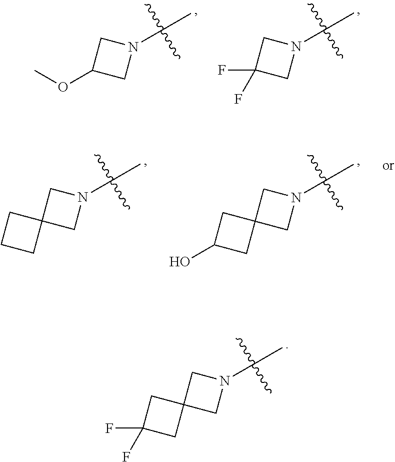

In some embodiments of the compounds of Formula II and stereoisomers and pharmaceutically acceptable salts thereof, one or more of R^{2a1}, R^{2a2}, R^{2a3}, and R^{2a4} is aryl or heteroaryl. In some embodiments, R^{2a1} is aryl or heteroaryl. In some embodiments, R^{2a2} is aryl or heteroaryl. In some embodiments, R^{2a3} is aryl or heteroaryl. In some embodiments, R^{2a4} is aryl or heteroaryl. In some embodiments, the aryl or heteroaryl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, R^{2a1}, R^{2a2}, R^{2a3}, or R^{2a4} is

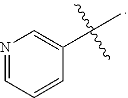 [SMILES: *c1cccnc1]

In some embodiments of the compounds of Formula II and stereoisomers and pharmaceutically acceptable salts thereof, one or more of R^{2a1}, R^{2a2}, R^{2a3}, and R^{2a4} is —O—C₁₋₆alkyl. In some embodiments, R^{2a1} is —O—C₁₋₆alkyl. In some embodiments, R^{2a2} is —O—C₁₋₆alkyl. In some embodiments, R^{2a3} is —O—C₁₋₆alkyl. In some embodiments, R^{2a4} is —O—C₁₋₆alkyl. In some embodiments, the —O—C₁₋₆alkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O—C₁₋₆alkyl is substituted with halo. In some embodiments, the —O—C₁₋₆alkyl is substituted with cycloalkyl. In some embodiments, R^{2a1}, R^{2a2}, R^{2a3}, or R^{2a4} is —OCH₃, —OCHF₂, —OCH₂CF₃, or

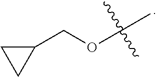 [SMILES: *OCC1CC1]

In some embodiments of the compounds of Formula II and stereoisomers and pharmaceutically acceptable salts thereof, one or more of R^{2a1}, R^{2a2}, R^{2a3}, and R^{2a4} is —O—C₂₋₆alkenyl. In some embodiments, R^{2a1} is —O—C₂₋₆alkenyl. In some embodiments, R^{2a2} is —O—C₂₋₆alkenyl. In some embodiments, R^{2a3} is —O—C₂₋₆ alkenyl. In some embodiments, R^{2a4} is —O—C₂₋₆alkenyl. In some embodiments, the —O—C₂₋₆alkenyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O—C₂₋₆alkenyl is substituted with halo.

In some embodiments of the compounds of Formula II and stereoisomers and pharmaceutically acceptable salts thereof, one or more of R^{2a1}, R^{2a2}, R^{2a3}, and R^{2a4} is —O—C₂₋₆-alkynyl. In some embodiments, R^{2a1} is —O—C₂₋₆alkynyl. In some embodiments, R^{2a2} is —O—C₂₋₆alkynyl. In some embodiments, R^{2a3} is —O—C₂₋₆alkynyl. In some embodiments, R^{2a4} is —O—C₂₋₆alkynyl. In some embodiments, the —O—C₂₋₆alkynyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O—C₂₋₆alkynyl is substituted with halo. In some embodiments, R^{2a1}, R^{2a2}, R^{2a3}, or R^{2a4} is

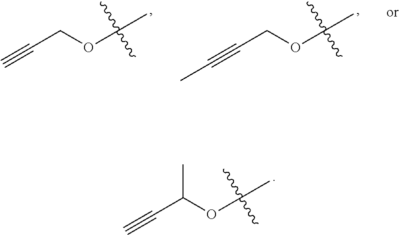

In some embodiments of the compounds of Formula II and stereoisomers and pharmaceutically acceptable salts thereof, one or more of R^{2a1}, R^{2a2}, R^{2a3}, and R^{2a4} is —O-cycloalkyl. In some embodiments, R^{2a1} is —O-cycloalkyl. In some embodiments, R^{2a2} is —O-cycloalkyl. In some embodiments, R^{2a3} is —O-cycloalkyl. In some embodiments, R^{2a4} is cycloalkyl. In some embodiments, the —O-cycloalkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-cycloalkyl is substituted with halo. In some embodiments, the —O-cycloalkyl is substituted with C₁₋₆haloalkyl. In some embodiments, R^{2a1}, R^{2a2}, R^{2a3}, or R^{2a4} is

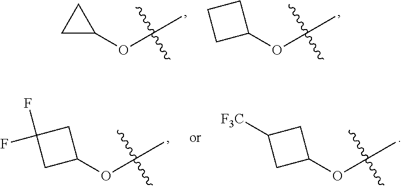 [SMILES: *OC1CC1 ; *OC1CCC1 ; *OC1CC(F)(F)C1 ; *OC1CC(C(F)(F)F)C1]

In some embodiments of the compounds of Formula II and stereoisomers and pharmaceutically acceptable salts thereof, one or more of R^{2a1}, R^{2a2}, R^{2a3}, and R^{2a4} is —O-heterocyclyl. In some embodiments, R^{2a1} is —O-heterocyclyl. In some embodiments, R^{2a2} is —O-heterocyclyl. In some embodiments, R^{2a3} is —O-heterocyclyl. In some embodiments, R^{2a4} is —O-heterocyclyl. In some embodiments, the —O-heterocyclyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-heterocyclyl is substituted with halo.

In some embodiments of the compounds of Formula II and stereoisomers and pharmaceutically acceptable salts thereof, one or more of R^{2a1}, R^{2a2}, R^{2a3}, and R^{2a4} is —O-aryl. In some embodiments, R^{2a1} is —O-aryl. In some embodiments, R^{2a2} is —O-aryl. In some embodiments, R^{2a3} is —O-aryl. In some embodiments, R^{2a4} is —O-aryl. In some embodiments, the —O-aryl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-aryl is substituted with halo. In some embodiments, the —O-aryl is substituted with C₁₋₆alkyl. In some embodiments, the —O-aryl is substituted with —C(O)NH₂. In some embodiments, the aryl of is a 6 membered monocyclic aryl. In some embodiments, R^{2a3} is —O-aryl wherein the aryl is an unsubstituted 6-membered monocyclic aryl. In some embodiments, R^{2a1}, R^{2a2}, R^{2a3}, or R^{2a4} is

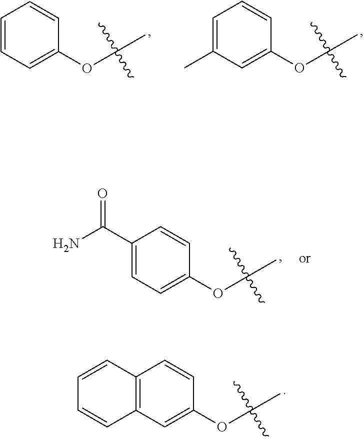

In some embodiments of the compounds of Formula II and stereoisomers and pharmaceutically acceptable salts thereof, one or more of R^{2a1}, R^{2a2}, R^{2a3}, and R^{2a4} is —O-heteroaryl. In some embodiments, R^{2a1} is —O-heteroaryl. In some embodiments, R^{2a2} is —O-heteroaryl. In some embodiments, R^{2a3} is —O-heteroaryl. In some embodiments, R^{2a4} is —O-heteroaryl. In some embodiments, the —O-heteroaryl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-heteroaryl is substituted with halo. In some embodiments, R^{2a1}, R^{2a2}, R^{2a3}, or R^{2a4} is

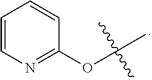 [SMILES: *Oc1ccccn1]

In some embodiments of the compounds of Formula II and stereoisomers and pharmaceutically acceptable salts thereof, one or more of R^{2a1}, R^{2a2}, R^{2a3}, and R^{2a4} is —SO₂-cycloalkyl. In some embodiments, R^{2a1} is —SO₂-cycloalkyl. In some embodiments, R^{2a2} is —SO₂-cycloalkyl. In some embodiments, R^{2a3} is —SO₂-cycloalkyl. In some embodiments, R^{2a4} is —SO₂-cycloalkyl. In some embodiments, the —SO₂-cycloalkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —SO₂-cycloalkyl is substituted with halo. In some embodiments, R^{2a1}, R^{2a2}, R^{2a3}, or R^{2a4} is

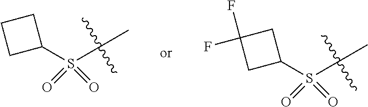 [SMILES: *S(=O)(=O)C1CCC1 ; *S(=O)(=O)C1CC(F)(F)C1]

In some embodiments of the compounds of Formula II and stereoisomers and pharmaceutically acceptable salts thereof, one or more of R^{2a1}, R^{2a2}, R^{2a3}, and R^{2a4} is —NR^{2L1}R^{2L2}. In some embodiments, R^{2a1} is —NR^{2L1}R^{2L2}. In some embodiments, R^{2a2} is —NR^{2L1}R^{2L2}. In some embodiments, R^{2a3} is —NR^{2L1}R^{2L2}. In some embodiments, R^{2a4} is —NR^{2L1}R^{2L2}. In some embodiments, R^{2a1}, R^{2a2}, R^{2a3}, or R^{2a4} is

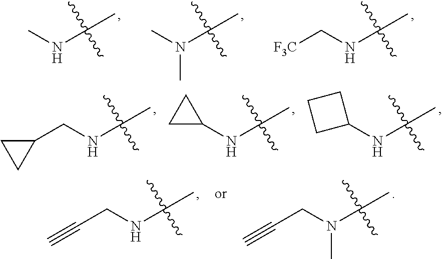

In some embodiments of the compounds of Formula II and stereoisomers and pharmaceutically acceptable salts thereof, R^{2L1} and R^{2L2} are independently H, C₁₋₆alkyl, C₂₋₆alkynyl, or cycloalkyl. In some embodiments, R^{2L1} is H. In some embodiments, R^{2L1} is H and R^{2L2} is C₁₋₆alkyl, C₂₋₆alkynyl, or cycloalkyl. In some embodiments, R^{2L1} is —CH₃. In some embodiments, R^{2L1} is —CH₃ and R^{2L2} is C₁₋₆alkyl, C₂₋₆alkynyl, or cycloalkyl. In some embodiments, R^{2L1} is H or —CH₃ and R^{2L2} is C₁₋₆alkyl. In some embodiments, R^{2L1} is H or —CH₃ and R^{2L2} is C₂₋₆alkynyl. In some embodiments, R^{2L1} is H or —CH₃ and R^{2L2} is cycloalkyl. In some embodiments, the C₁₋₆alkyl, C₂₋₆alkynyl, or cycloalkyl is unsubstituted or substituted with one or more substituents independently selected from the group consisting of halo, C₁₋₆alkyl, C₁₋₆haloalkyl, cycloalkyl, and halocycloalkyl. In some embodiments, the C₁₋₆alkyl is substituted with halo. In some embodiments, the C₁₋₆alkyl is substituted with cycloalkyl.

In some embodiments of the compounds of Formula II and stereoisomers and pharmaceutically acceptable salts thereof, one or more of R^{2a1}, R^{2a2}, R^{2a3}, and R^{2a4} is C₂₋₆alkynyl-NR^{2L3}R^{2L4}. In some embodiments, R^{2a1} is C₂₋₆alkynyl-NR^{2L3}R^{2L4}. In some embodiments, R^{2a2} is C₂₋₆alkynyl-NR^{2L3}R^{2L4}. In some embodiments, R^{2a3} is C₂₋₆alkynyl-NR^{2L3}R^{2L4}. In some embodiments, R^{2a4} is C₂₋₆-alkynyl-NR^{2L3}R^{2L4}.

In some embodiments of the compounds of Formula II and stereoisomers and pharmaceutically acceptable salts thereof, R^{2L3} and R^{2L4} are independently H, C₁₋₆alkyl, C₂₋₆alkynyl, or cycloalkyl. In some embodiments, R^{2L3} is H. In some embodiments, R^{2L3} is H and R^{2L4} is C₁₋₆alkyl, C₂₋₆alkynyl, or cycloalkyl. In some embodiments, R^{2L3} is —CH₃. In some embodiments, R^{2L3} is —CH₃ and R^{2L4} is C₁₋₆alkyl, C₂₋₆alkynyl, or cycloalkyl. In some embodiments, R^{2L3} is C₁₋₆alkyl and R^{2L4} is C₁₋₆alkyl. In some embodiments, R^{2L3} is H or —CH₃ and R^{2L4} is C₁₋₆alkyl. In some embodiments, R^{2L3} is H or —CH₃ and R^{2L4} is C₂₋₆alkynyl. In some embodiments, R^{2L3} is H or —CH₃ and R^{2L4} is cycloalkyl. In some embodiments, the C₁₋₆alkyl, C₂₋₆alkynyl, or cycloalkyl is unsubstituted or substituted with one or more substituents independently selected from the group consisting of halo, C₁₋₆alkyl, C₁₋₆haloalkyl, cycloalkyl, and halocycloalkyl. In some embodiments, the C₁₋₆alkyl is substituted with halo. In some embodiments, the C₁₋₆alkyl is substituted with cycloalkyl. In some embodiments, R^{2a1}, R^{2a2}, R^{2a3}, or R^{2a4} is

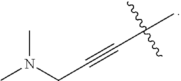 [SMILES: *C#CCN(C)C]

In some embodiments of the compounds of Formula II and stereoisomers and pharmaceutically acceptable salts thereof, two or more of R^{2a1}, R^{2a2}, R^{2a3}, and R^{2a4} is not H. In some embodiments, R^{2a3} is not H and one of R^{2a1}, R^{2a2}, or R^{2a4} is not H. In some embodiments, R^{2a2} is F or Cl and R^{2a3} is —O—C₁₋₆alkyl or —O-cycloalkyl, wherein the —O—C₁₋₆alkyl or —O-cycloalkyl is unsubstituted or substituted with halo or —CN. In some embodiments, R^{2a1} is not H and R^{2a2} is not H. In some embodiments, R^{2a1} is F or Cl and R^{2a2} is F or Cl.

In some embodiments of the compounds of Formula II and stereoisomers and pharmaceutically acceptable salts thereof. X³ is —O—. In some embodiments, X³ is —NR^{2b1}—. In some embodiments, X³ is —NH—. In some embodiments, X³ is —N(CH₃)—. In some embodiments, X³ is —CR^{2b2}R^{2b3}—. In some embodiments, X³ is —CH(CH₃)—. In some embodiments, X³ is —CD₂-. In some embodiments, X³ is —CH₂—.

In some embodiments of the compounds of Formula II and stereoisomers and pharmaceutically acceptable salts thereof. A2 is

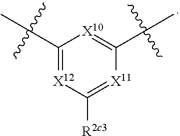

In some embodiments, one or more of X¹⁰, X¹¹, and X¹² is —N—. In some embodiments, X¹⁰ is —N—. In some embodiments, X¹¹ is —N—. In some embodiments, X¹² is —N—. In some embodiments, X¹⁰ is —CR^{2c1}—. In some embodiments, X¹¹ is —CR^{2c2}—. In some embodiments, X¹² is —CR^{2c3}—. In some embodiments, X¹⁰ is —CR^{2c1}—, X¹¹ is —CR^{2c2}—, and X¹² is —CR^{2c3}—.

In some embodiments of the compounds of Formula II and stereoisomers and pharmaceutically acceptable salts thereof, one or more of R^{2c1}, R^{2c2}, R^{2c3}, and R^{2c4} is not H. In some embodiments, one or more of R^{2c1}, R^{2c2}, R^{2c3}, and R^{2c4} is —CN. In some embodiments, one or more of R^{2c1}, R^{2c2}, R^{2c3}, and R^{2c4} is independently Cl, F, or Br. In some embodiments, two or more of R^{2c1}, R^{2c2}, R^{2c3}, and R^{2c4} are independently Cl, F, or Br. In some embodiments, R^{2c2} is F and R^{2c1}, R^{2c3}, and R^{2c4} are H. In some embodiments, R^{2c1}, R^{2c2}, R^{2c3}, and R^{2c4} are H.

In some embodiments of the compounds of Formula II and stereoisomers and pharmaceutically acceptable salts thereof. A2 is

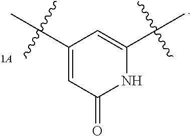 [SMILES: "." ; *c1cc(*)[nH]c(=O)c1]

In some embodiments, A2 is

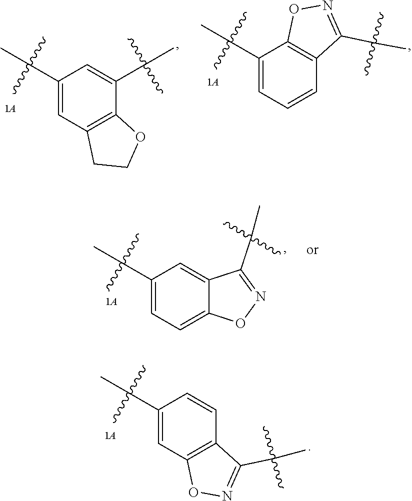 [SMILES: *c1cc(*)c2c(c1)CCO2 ; *c1noc2c(*)cccc12 ; *c1ccc2onc(*)c2c1 ; *c1ccc2c(*)noc2c1]

In some embodiments, A2 is

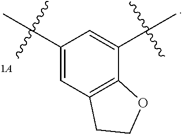 [SMILES: *c1cc(*)c2c(c1)CCO2]

In some embodiments, A2 is

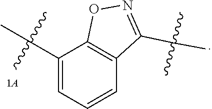 [SMILES: *c1noc2c(*)cccc12]

In some embodiments, A2 is

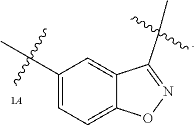 [SMILES: *c1ccc2onc(*)c2c1]

In some Embodiments, A2 is

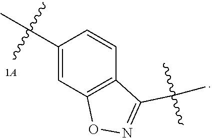 [SMILES: *c1ccc2c(*)noc2c1]

In some embodiments of the compounds of Formula II and stereoisomers and pharmaceutically acceptable salts thereof, B4, B5, or B6 is a 3-membered monocyclic heterocyclediyl, a 4-membered monocyclic heterocyclediyl, a 5-membered monocyclic heterocyclediyl comprising 2 or more N, a 6-membered monocyclic heterocyclediyl comprising 2 or more N, a 7-membered monocyclic heterocyclediyl, an 8-membered monocyclic heterocyclediyl, a 7 to 18-membered polycyclic heterocyclediyl, or a 7 to 18-membered spirocyclic heterocyclediyl, wherein the 3-membered monocyclic heterocyclediyl, 4-membered monocyclic heterocyclediyl, 5-membered monocyclic heterocyclediyl, 6-membered monocyclic heterocyclediyl, 7-membered monocyclic heterocyclediyl, 8-membered monocyclic heterocyclediyl, 7 to 18-membered polycyclic heterocyclediyl, or 7 to 18-membered spirocyclic heterocyclediyl of B4, B5, or B6 is unsubstituted or substituted with one or more substituents independently selected from the group consisting of halo, C₁₋₆alkyl, and oxo. In some embodiments, the 7 to 18-membered polycyclic heterocyclediyl is a 7, 8, 9, 10, 11, 12, 13, 14, 15, 16, 17, or 18-membered polycyclic heterocyclediyl. In some embodiments, the 7 to 18-membered spirocyclic heterocyclediyl is a 7, 8, 9, 10, 11, 12, 13, 14, 15, 16, 17, or 18-membered spirocyclic heterocyclediyl.

In some embodiments of the compounds of Formula II and stereoisomers and pharmaceutically acceptable salts thereof, B4, B5, or B6 is a 3 to 8-membered monocyclic heterocyclediyl, wherein the 3 to 8-membered monocyclic heterocyclediyl is unsubstituted or substituted with one or more substituents selected from the group consisting of halo, C₁₋₆alkyl, and oxo. In some embodiments, the 3 to 8-membered monocyclic heterocyclediyl is a 3, 4, 5, 6, 7, or 8-membered monocyclic heterocyclediyl. In some embodiments, B4, B5, or B6 is a 4-membered monocyclic heterocyclediyl, wherein the 4-membered monocyclic heterocyclediyl is unsubstituted or substituted with one or more substituents selected from the group consisting of halo, C₁₋₆alkyl, and oxo. In some embodiments, B4, B5, or B6 is

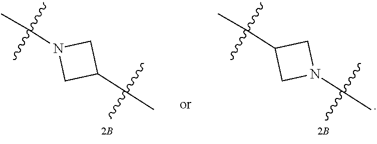 [SMILES: *C1CN(*)C1 ; *C1CN(*)C1]

In some embodiments, B4, B5, or B6 is a 6-membered monocyclic heterocyclediyl, wherein the 6-membered monocyclic heterocyclediyl is unsubstituted or substituted with one or more substituents selected from the group consisting of halo, C₁₋₆alkyl, and oxo. In some embodiments, B4, B5, or B6 is

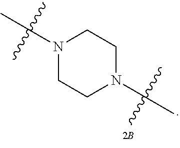 [SMILES: *N1CCN(*)CC1]

In some embodiments, B4, B5, or B6 is a 7-membered monocyclic heterocyclediyl, wherein the 7-membered monocyclic heterocyclediyl is unsubstituted or substituted with one or more substituents selected from the group consisting of halo, C₁₋₆alkyl, and oxo. In some embodiments, B4, B5, or B6 is

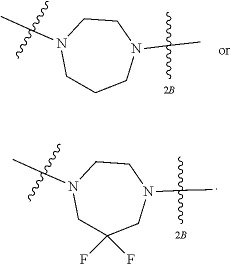 [SMILES: *N1CCCN(*)CC1 ; *N1CCN(*)CC(F)(F)C1]

In some embodiments of the compounds of Formula II and stereoisomers and pharmaceutically acceptable salts thereof, B4, B5, or B6 is a 7 to 18-membered polycyclic heterocyclediyl, wherein the 7 to 18-membered poly cyclic heterocyclediyl is unsubstituted or substituted with one or more substituents selected from the group consisting of halo, C₁₋₆alkyl, and oxo. In some embodiments, the 7 to 18-membered polycyclic heterocyclediyl is a 7, 8, 9, 10, 11, 12, 13, 14, 15, 16, 17, or 18-membered polycyclic heterocyclediyl. In some embodiments, B4, B5, or B6 is

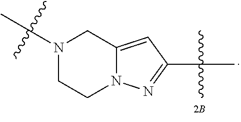 [SMILES: *c1cc2n(n1)CCN(*)C2]

In some Embodiments, B4, B5, or B6 is

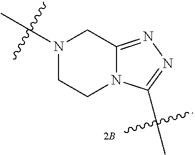 [SMILES: *c1nnc2n1CCN(*)C2]

In some embodiments B4, B5, or B6 is

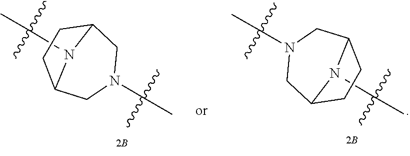

In some embodiments, B4, B5, or B6 is

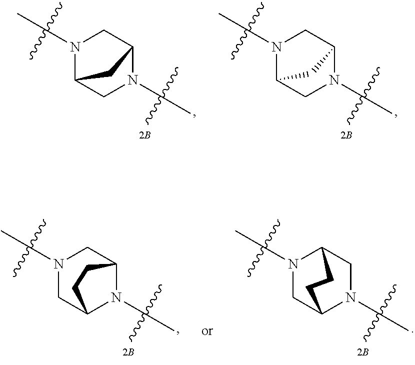

In some embodiments, B4, B5, or B6 is

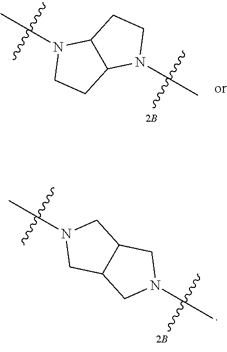 [SMILES: *N1CCC2C1CCN2* ; *N1CC2CN(*)CC2C1]

In some embodiments, B4, B5, or B6 is

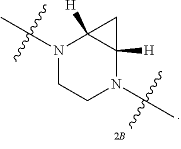 [SMILES: *N1CCN(*)[C@H]2C[C@H]21]

In some embodiments of the compounds of Formula II and stereoisomers and pharmaceutically acceptable salts thereof, B4, B5, or B6 is a 7 to 18-membered spirocyclic heterocyclediyl, wherein the 7 to 18-membered spirocyclic heterocyclediyl is unsubstituted or substituted with one or more substituents selected from the group consisting of halo, C₁₋₆alkyl, and oxo. In some embodiments, the 7 to 18-membered spirocyclic heterocyclediyl is a 7, 8, 9, 10, 11, 12, 13, 14, 15, 16, 17, or 18-membered spirocyclic heterocyclediyl. In some embodiments, B4, B5, or B6 is

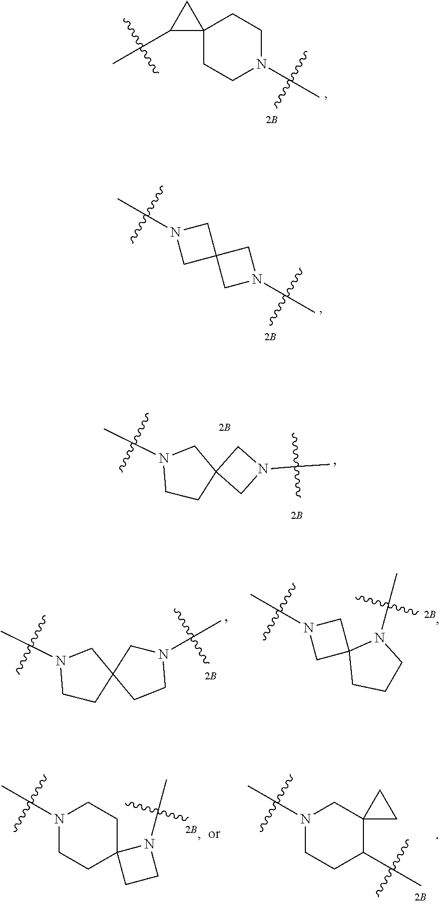

In some embodiments of the compounds of Formula II and stereoisomers and pharmaceutically acceptable salts thereof, the monocyclic heterocyclediyl, polycyclic heterocyclediyl, or spirocyclic heterocyclediyl of B4, B5, or B6 comprises one or more N. In some embodiments, the monocyclic heterocyclediyl, polycyclic heterocyclediyl, or spirocyclic heterocyclediyl of B4, B5, or B6 comprises two or more N.

In some embodiments of the compounds of Formula II and stereoisomers and pharmaceutically acceptable salts thereof, D4, D5, D6, or D7 is C₁₋₆alkyl, wherein the C₁₋₆alkyl is unsubstituted or substituted with one or more substituents selected from the group consisting of halo, —CN, C₁₋₆alkyl, C₁₋₆ haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{2D10})₂. In some embodiments, the C₁₋₆alkyl is unsubstituted or substituted with halo. In some embodiments, the C₁₋₆alkyl is unsubstituted or substituted with —OH. In some embodiments, the C₁₋₆alkyl is unsubstituted or substituted with —O—C₁₋₆alkyl.

In some embodiments of the compounds of Formula II and stereoisomers and pharmaceutically acceptable salts thereof, D4, D5, D6, or D7 is cycloalkyl, wherein the cycloalkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{2D10})₂. In some embodiments, the cycloalkyl is unsubstituted or substituted with halo. In some embodiments, D4, D5, D6, or D7 is

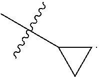 [SMILES: *C1CC1]

In some embodiments of the compounds of Formula II and stereoisomers and pharmaceutically acceptable salts thereof, D4, D5, D6, or D7 is aryl, wherein the aryl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{2D10})₂. In some embodiments, the aryl is a monocyclic 6-membered aryl. In some embodiments, the aryl is substituted with one or more substituents selected from the group consisting of halo, —CN, C₁₋₆alkyl, and C₁₋₆haloalkyl. In some embodiments, the aryl is substituted with —CN.

In some embodiments of the compounds of Formula II and stereoisomers and pharmaceutically acceptable salts thereof, D4, D5, D6, or D7 is heteroaryl, wherein the heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, —C(O)NH₂, —C(O)NHCH₃, and —C(O)N(CH₃)₂.

In some embodiments of the compounds of Formula II and stereoisomers and pharmaceutically acceptable salts thereof, D4, D5, D6, or D7 is a monocyclic 5 or 6-membered heteroaryl comprising one or more N, wherein the monocyclic 5 or 6-membered heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, —C(O)NH₂, —C(O)NHCH₃, and —C(O)N(CH₃)₂. In some embodiments, the monocyclic 5 or 6-membered heteroaryl is substituted with —CN. In some embodiments, the monocyclic 5 or 6-membered heteroaryl is substituted with —CF₃. In some embodiments, the monocyclic 5 or 6-membered heteroaryl is substituted with —OCH₃.

In some embodiments of the compounds of Formula II and stereoisomers and pharmaceutically acceptable salts thereof, D4, D5, D6, or D7 is —O—C₁₋₆alkyl, wherein the —O—C₁₋₆alkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆-haloalkyl, —C(O)-cycloalkyl, and —C(O)N(R^{2D10})₂. In some embodiments, the —O—C₁₋₆alkyl is unsubstituted or substituted with halo.

In some embodiments of the compounds of Formula II and stereoisomers and pharmaceutically acceptable salts thereof, D4, D5, D6, or D7 is —O-aryl or —O-heteroaryl, wherein the —O-aryl or —O-heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆ alkyl-O—C₁₋₆alkyl, C₁₋₆alkyl-O—C₁₋₆haloalkyl, —C(O)-cycloalkyl, and —C(O)N(R^{2D10})₂. In some embodiments, the —O-aryl or —O-heteroaryl is unsubstituted or substituted with halo. In some embodiments, the —O-aryl or —O-heteroaryl is unsubstituted or substituted with —CN. In some Embodiments, D4, D5, D6, or D7 is

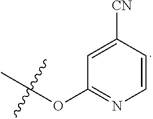 [SMILES: *Oc1cc(C#N)ccn1]

In some embodiments of the compounds of Formula II and stereoisomers and pharmaceutically acceptable salts thereof, D4, D5, D6, or D7 is —C(O)—C₁₋₆alkyl, wherein the —C(O)—C₁₋₆alkyl is unsubstituted or substituted with one or more substituents selected from the group consisting of halo, —CN, C₁₋₆alkyl, C₁₋₆haloalkyl, cycloalkyl, —OH, —C₁₋₆alkyl, C₁₋₆alkyl-OH—, —O—C₁₋₆haloalkyl, C₁₋₆alkyl-O—C₁₋₆ alkyl, C₁₋₆alkyl-O—C₁₋₆haloalkyl, —C(O)-cycloalkyl, and —C(O)N(R^{2D10})₂. In some embodiments, the —C(O)—C₁₋₆alkyl is unsubstituted or substituted with halo. In some embodiments, the —C(O)—C₁₋₆alkyl is unsubstituted or substituted with —CN. In some embodiments, D4, D5, D6, or D7 is

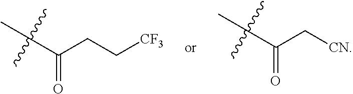 [SMILES: *C(=O)CCC(F)(F)F ; *C(=O)CC#N]

In some embodiments of the compounds of Formula II and stereoisomers and pharmaceutically acceptable salts thereof, D4, D5, D6, or D7 is —C(O)-cycloalkyl, wherein the —C(O)-cycloalkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆ alkyl, C₁₋₆alkyl-O—C₁₋₆haloalkyl, —C(O)-cycloalkyl, and —C(O)N(R^{2D10})₂. In some embodiments, the —C(O)-cycloalkyl is unsubstituted or substituted with halo. In some embodiments, D4, D5, D6, or D7 is

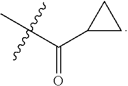 [SMILES: *C(=O)C1CC1]

In some embodiments of the compounds of Formula II and stereoisomers and pharmaceutically acceptable salts thereof, D4, D5, D6, or D7 is —C(O)-heterocyclyl, wherein the —C(O)-heterocyclyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{2D10})₂. In some embodiments, the —C(O)-heterocyclyl is unsubstituted or substituted with halo. In some embodiments, D4, D5, D6, or D7 is

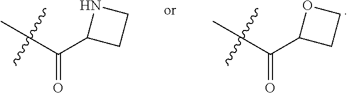 [SMILES: *C(=O)C1CCN1 ; *C(=O)C1CCO1]

In some embodiments of the compounds of Formula II and stereoisomers and pharmaceutically acceptable salts thereof, D4, D5, D6, or D7 is —C(O)-aryl or —C(O)-heteroaryl, wherein the —C(O)-aryl or —C(O)-heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆ haloalkyl, C₁₋₆alkyl-O—C₁₋₆alkyl, C₁₋₆alkyl-O—C₁₋₆ haloalkyl, —C(O)-cycloalkyl, and —C(O)N(R^{2D10})₂. In some embodiments, the —C(O)-aryl or —C(O)-heteroaryl is unsubstituted or substituted with halo. In some embodiments, the —C(O)-aryl or —C(O)-heteroaryl is unsubstituted or substituted with —CF₃. In some embodiments, the —C(O)-aryl or —C(O)-heteroaryl is unsubstituted or substituted with —CN. In some embodiments, D4, D5, D6, or D7 is

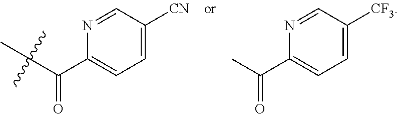

In some embodiments of the compounds of Formula II and stereoisomers and pharmaceutically acceptable salts thereof, D4, D5, D6, or D7 is —N(R^{2D1})(R^{2D2}). In some embodiments, D4, D5, D6, or D7 is

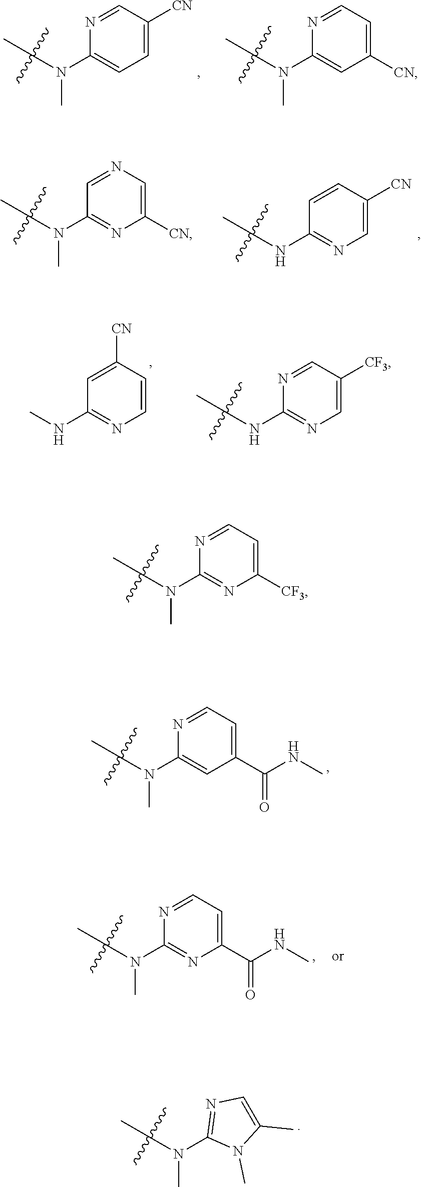

In some embodiments of the compounds of Formula II and stereoisomers and pharmaceutically acceptable salts thereof, R^{2D1} is H. In some embodiments, R^{2D1} is C₁₋₆alkyl. In some embodiments, R^{2D2} is aryl. In some embodiments, R^{2D2} is heteroaryl. In some embodiments, the heteroaryl is a 6-membered heteroaryl.

In some embodiments of the compounds of Formula II and stereoisomers and pharmaceutically acceptable salts thereof, D4, D5, D6, or D7 is —C(O)N(R^{2D3})(R^{2D4}). In some embodiments, D4, D5, D6, or D7 is

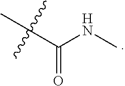

In some embodiments of the compounds of Formula II and stereoisomers and pharmaceutically acceptable salts thereof, R^{2D3} is H. In some embodiments, R^{2D3} is C₁₋₆alkyl. In some embodiments, R^{2D4} is C₁₋₆alkyl. In some embodiments, R^{2D3} is H and R^{2D4} is C₁₋₆alkyl.

In some embodiments of the compounds of Formula II and stereoisomers and pharmaceutically acceptable salts thereof, D4, D5, D6, or D7 is —N(R^{2D5})C(O)R^{2D6}. In some embodiments, D4, D5, D6, or D7 is

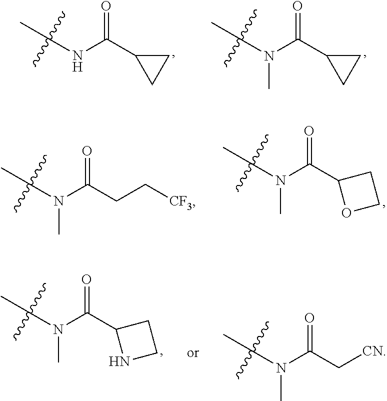

In some embodiments of the compounds of Formula H and stereoisomers and pharmaceutically acceptable salts thereof, R^{2D5} is H. In some embodiments, R^{2D5} is C₁₋₆alkyl. In some embodiments, R^{2D6} is C₁₋₆alkyl. In some embodiments, R^{2D6} is cycloalkyl. In some embodiments, R^{2D6} is heterocyclyl. In some embodiments, R^{2D5} is H and R^{2D6} is C₁₋₆alkyl, cycloalkyl, or heterocyclyl.

In some embodiments, the compound is of Formula III:

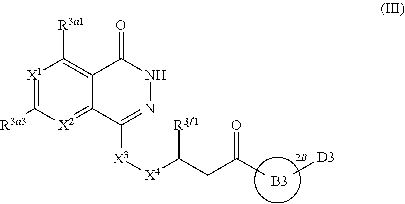

and stereoisomers and pharmaceutically acceptable salts thereof, wherein:
X¹ is —N— or —CR^{3a2}—;
X² is —N— or —CR^{3a4}—;
R^{3a1}, R^{3a2}, R^{3a3}, and R^{3a4} are independently H, halo, —OH, C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, —SO₂-cycloalkyl, —NR^{3L1}R^{3L2}, or C₂₋₆alkynyl-NR^{3L3}R^{3L4};
wherein if X¹ is —CR^{3a2}— and X² is —CR^{3a4}—, then at least one of R^{3a1}, R^{3a2}, R^{3a3}, and R^{3a4} is not H;
wherein each C₁₋₆alkyl, C₂₋₆alkenyl, C₂₋₆alkynyl, cycloalkyl, heterocyclyl, aryl, heteroaryl, —O—C₁₋₆alkyl. O—C₂₋₆alkenyl, —O—C₂₋₆alkynyl, —O-cycloalkyl, —O-heterocyclyl, —O-aryl, —O-heteroaryl, or —SO₂-cycloalkyl of R^{3a1}, R^{3a2}, R^{3a3}, and R^{3a4} is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂;
R^{3L1}, R^{3L2}, R^{3L3} and R^{3L4} are independently H, C₁₋₆alkyl, C₂₋₆alkynyl, or cycloalkyl, wherein each C₁₋₆alkyl, C₂₋₆alkynyl, or cycloalkyl of R^{3L1}, R^{3L2}, R^{3L3}, and R^{3L4} is unsubstituted or substituted with one or more substituents independently selected from the group consisting of halo, C₁₋₆alkyl, C₁₋₆haloalkyl, cycloalkyl, and halocycloalkyl;
X³ is —O—, —NR^{3b1}—, or —CR^{3b2}R^{3b3}—, wherein R^{3b1}, R^{3b2}, and R^{3b3} are independently H or C₁₋₆alkyl;
R^{3f1} is H or C₁₋₆alkyl;
X⁴ is —O—, —NR^{3f2}—, or —CR^{3f3}R^{3f4}—, wherein R^{3f2}, R^{3f3}, and R^{3f4} are independently selected from H and C₁₋₆alkyl;
B3 is a 3 to 8-membered monocyclic heterocyclediyl, a 7 to 18-membered polycyclic heterocyclediyl, or a 7 to 18-membered spirocyclic heterocyclediyl;
wherein the 3 to 8-membered monocyclic heterocyclediyl, 7 to 18-membered polycyclic heterocyclediyl, or 7 to 18-membered spirocyclic heterocyclediyl of B3 is unsubstituted or substituted with one or more substituents selected from the group consisting of halo, C₁₋₆alkyl, and oxo;
D3 is C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, —C(O)-heteroaryl, —N(R^{3D1})(R^{3D2}), —C(O)N(R^{3D3})(R^{3D4}), or —N(R^{3D5})C(O)R^{3D6};
wherein the C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, or —C(O)-heteroaryl of D3 is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{3D10})₂, wherein each R^{3D10} is independently H or C₁₋₆alkyl;
R^{3D1}, R^{3D3}, and R^{3D5} are independently H or C₁₋₆alkyl;
R^{3D2} is aryl or heteroaryl, wherein the aryl or heteroaryl of R^{3D2} is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{3D11})₂, wherein each R^{3D11} is independently H or C₁₋₆alkyl; and
R^{3D4} and R^{3D6} are independently C₁₋₆alkyl, cycloalkyl, heterocyclyl, aryl, or heteroaryl, wherein each C₁₋₆alkyl, cycloalkyl, heterocyclyl, aryl, or heteroaryl of R^{3D4} and R^{3D6} is unsubstituted or substituted with one or more substituents independently selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{3D12})₂, wherein each R^{3D12} is independently H or C₁₋₆alkyl.

In some embodiments of the compounds of Formula III and stereoisomers and pharmaceutically acceptable salts thereof, X¹ is —CR^{3a2}—. In some embodiments, X² is —CR^{3a4}—. In some embodiments, X¹ is —N—. In some embodiments, X² is —N—. In some embodiments, X¹ is —CR^{3a2}— and X² is —CR^{3a4}—. In some embodiments. X¹ is —CR^{3a2}— and X² is —N—. In some embodiments, X¹ is —N— and X² is —CR^{3a4}—.

In some embodiments of the compounds of Formula III and stereoisomers and pharmaceutically acceptable salts thereof, one or more of R^{3a1}, R^{3a2}, R^{3a3}, and R^{3a4} is not H. In some embodiments, R^{3a1} is not H. In some embodiments, R^{3a2} is not H. In some embodiments, R^{3a3} is not H. In some embodiments, R^{3a4} is not H.

In some embodiments of the compounds of Formula III and stereoisomers and pharmaceutically acceptable salts thereof, one or more of R^{3a1}, R^{3a2}, R^{3a3}, and R^{3a4} is halo. In some embodiments, R^{3a1} is halo. In some embodiments, R^{3a2} is halo. In some embodiments, R^{3a3} is halo. In some embodiments, R^{3a4} is halo. In some embodiments, the halo is F, Cl, or Br. In some embodiments, the halo is Cl. In some embodiments, the halo is F. In some embodiments, R^{3a1} is F. In some embodiments, R^{3a3} is F. In some embodiments, R^{3a3} is F.

In some embodiments of the compounds of Formula III and stereoisomers and pharmaceutically acceptable salts thereof, one or more of R^{3a1}, R^{3a2}, R^{3a3}, and R^{3a4} is —OH. In some embodiments, R^{3a1} is —OH. In some embodiments, R^{3a2} is —OH. In some embodiments, R^{3a3} is —OH. In some embodiments, R^{3a4} is —OH.

In some embodiments of the compounds of Formula III and stereoisomers and pharmaceutically acceptable salts thereof, one or more of R^{3a1}, R^{3a2}, R^{3a3}, and R^{3a4} is C₁₋₆alkyl. In some embodiments, R^{3a1} is C₁₋₆alkyl. In some embodiments, R^{3a2} is C₁₋₆alkyl. In some embodiments, R^{3a3} is C₁₋₆alkyl. In some embodiments, R^{3a4} is C₁₋₆alkyl. In some embodiments, the C₁₋₆alkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the C₁₋₆alkyl is substituted with halo. In some embodiments, R^{3a1}, R^{3a2}, R^{3a3}, or R^{3a4} is —CH₃, —CH₂CH₃, —CH₂CF₃, —CF₃, —CF₂CH₃, —CH₂CH₂CF₃,

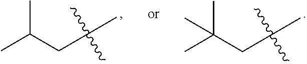

In some embodiments of the compounds of Formula III and stereoisomers and pharmaceutically acceptable salts thereof, one or more of R^{3a1}, R^{3a2}, R^{3a3}, and R^{3a4} is C₂₋₆alkenyl. In some embodiments, R^{3a1} is C₂₋₆alkenyl. In some embodiments, R^{3a2} is C₂₋₆alkenyl. In some embodiments, R^{3a3} is C₂₋₆alkenyl. In some embodiments, R^{3a4} is C₂₋₆alkenyl. In some embodiments, the C₂₋₆alkenyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the C₂₋₆alkenyl is substituted with halo. In some embodiments, R^{3a1}, R^{3a2}, R^{3a3}, or R^{3a4} is

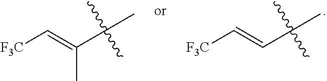

In some embodiments of the compounds of Formula III and stereoisomers and pharmaceutically acceptable salts thereof, one or more of R^{3a1}, R^{3a2}, R^{3a3}, and R^{3a4} is C₂₋₆alkynyl. In some embodiments, R^{3a1} is C₂₋₆alkynyl. In some embodiments, R^{3a2} is C₂₋₆alkynyl. In some embodiments, R^{3a3} is C₂₋₆alkynyl. In some embodiments, R^{3a4} is C₂₋₆alkynyl. In some embodiments, the C₂₋₆alkynyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the C₂₋₆alkynyl is substituted with halo. In some embodiments, the C₂₋₆₆alkynyl is substituted with cycloalkyl. In some embodiments, R^{3a1}, R^{3a2}, R^{3a3}, or R^{3a4} is

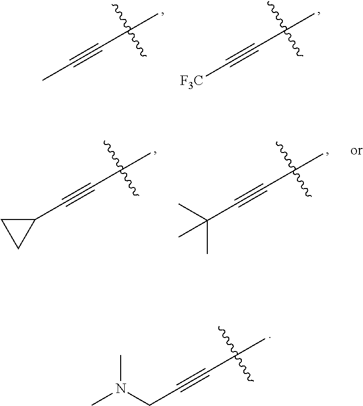

In some embodiments of the compounds of Formula III and stereoisomers and pharmaceutically acceptable salts thereof, one or more of R^{3a1}, R^{3a2}, R^{3a3}, and R^{3a4} is cycloalkyl. In some embodiments, R^{3a1} is cycloalkyl. In some embodiments, R^{3a2} is cycloalkyl. In some embodiments, R^{3a3} is cycloalkyl. In some embodiments, R^{3a4} is cycloalkyl. In some embodiments, the cycloalkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the cycloalkyl is substituted with halo. In some embodiments, R^{3a1}, R^{3a2}, R^{3a3}, or R^{3a4} is

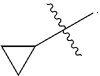 [SMILES: *C1CC1]

In some embodiments of the compounds of Formula III and stereoisomers and pharmaceutically acceptable salts thereof, one or more of R^{3a1}, R^{3a2}, R^{3a3}, and R^{3a4} is heterocyclyl. In some embodiments, R^{3a1} is heterocyclyl. In some embodiments, R^{3a2} is heterocyclyl. In some embodiments, R^{3a3} is heterocyclyl. In some embodiments, R^{3a4} is heterocyclyl. In some embodiments, the heterocyclyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the heterocyclyl is substituted with halo. In some embodiments, the heterocyclyl is substituted with —OH. In some embodiments, R^{3a1}, R^{3a2}, R^{3a3}, or R^{3a4} is

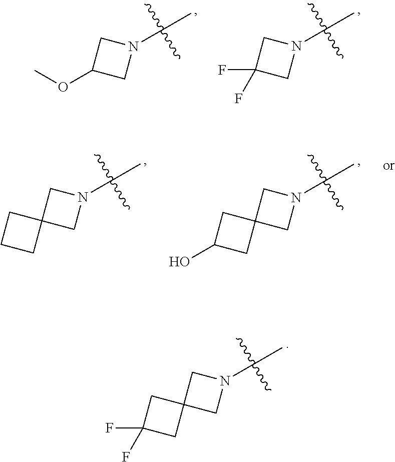

In some embodiments of the compounds of Formula III and stereoisomers and pharmaceutically acceptable salts thereof, one or more of R^{3a1}, R^{3a2}, R^{3a3}, and R^{3a4} is aryl or heteroaryl. In some embodiments, R^{3a1} is aryl or heteroaryl. In some embodiments, R^{3a2} is aryl or heteroaryl. In some embodiments, R^{3a3} is aryl or heteroaryl. In some embodiments, R^{3a4} is aryl or heteroaryl. In some embodiments, the aryl or heteroaryl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, R^{3a1}, R^{3a2}, R^{3a3}, or R^{3a4} is

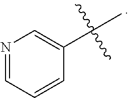 [SMILES: *c1cccnc1]

In some embodiments of the compounds of Formula III and stereoisomers and pharmaceutically acceptable salts thereof, one or more of R^{3a1}, R^{3a2}, R^{3a3}, and R^{3a4} is —O—C₁₋₆alkyl. In some embodiments, R^{3a1} is —O—C₁₋₆alkyl. In some embodiments, R^{3a2} is —O—C₁₋₆alkyl. In some embodiments, R^{3a3} is —O—C₁₋₆alkyl. In some embodiments, R^{3a4} is —O—C₁₋₆alkyl. In some embodiments, the —O—C₁₋₆alkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O—C₁₋₆alkyl is substituted with halo. In some embodiments, the —O—C₁₋₆alkyl is substituted with cycloalkyl. In some embodiments, R^{3a1}, R^{3a2}, R^{3a3}, or R^{3a4} is —OCH₃, —OCHF₂, —OCH₂CF₃, or

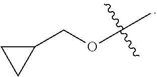 [SMILES: *OCC1CC1]

In some embodiments of the compounds of Formula III and stereoisomers and pharmaceutically acceptable salts thereof, one or more of R^{3a1}, R^{3a2}, R^{3a3}, and R^{3a4} is —O—C₂₋₆alkenyl. In some embodiments, R^{3a1} is —O—C₂₋₆alkenyl. In some embodiments, R^{3a2} is —O—C₂₋₆alkenyl. In some embodiments, R^{3a3} is —O—C₂₋₆alkenyl. In some embodiments, R^{3a4} is —O—C₂₋₆alkenyl. In some embodiments, the —O—C₂₋₆alkenyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O—C₂₋₆alkenyl is substituted with halo.

In some embodiments of the compounds of Formula III and stereoisomers and pharmaceutically acceptable salts thereof, one or more of R^{3a1}, R^{3a2}, R^{3a3}, and R^{3a4} is —O—C₂₋₆alkynyl. In some embodiments, R^{3a1} is —O—C₂₋₆alkynyl. In some embodiments, R^{3a2} is —O—C₂₋₆alkynyl. In some embodiments, R^{3a3} is —O—C₂₋₆alkynyl. In some embodiments, R^{3a4} is —O—C₂₋₆alkynyl. In some embodiments, the —O—C₂₋₆alkynyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O—C₂₋₆alkynyl is substituted with halo. In some embodiments, R^{3a1}, R^{3a2}, R^{3a3}, or R^{3a4} is

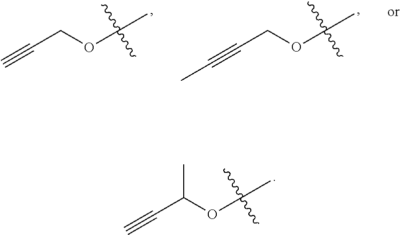

In some embodiments of the compounds of Formula III and stereoisomers and pharmaceutically acceptable salts thereof, one or more of R^{3a1}, R^{3a2}, R^{3a3}, and R^{3a4} is —O-cycloalkyl. In some embodiments, R^{1a1}, R^{2a1}, R^{3a1}, R^{4a1}, R^{5a1}, R^{6a1}, or R^{7a1} is —O-cycloalkyl. In some embodiments, R^{1a2}, R^{2a2}, R^{3a2}, R^{4a2}, R^{5a2}, R^{6a2}, or R^{7a2} is —O-cycloalkyl. In some embodiments, R^{3a3} is —O-cycloalkyl. In some embodiments, R^{3a4} is cycloalkyl. In some embodiments, the —O-cycloalkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-cycloalkyl is substituted with halo. In some embodiments, the —O-cycloalkyl is substituted with C₁₋₆haloalkyl. In some embodiments, R^{3a1}, R^{3a2}, R^{3a3}, or R^{3a4} is

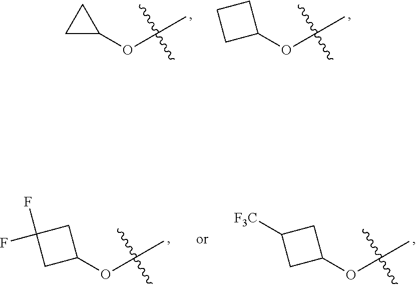 [SMILES: *OC1CC1 ; *OC1CCC1 ; *OC1CC(F)(F)C1 ; *OC1CC(C(F)(F)F)C1]

In some embodiments of the compounds of Formula III and stereoisomers and pharmaceutically acceptable salts thereof, one or more of R^{3a1}, R^{3a2}, R^{3a3}, and R^{3a4} is —O-heterocyclyl. In some embodiments, R^{3a1} is —O-heterocyclyl. In some embodiments, R^{3a2} is —O-heterocyclyl. In some embodiments, R^{3a3} is —O-heterocyclyl. In some embodiments, R^{3a4} is —O-heterocyclyl. In some embodiments, the —O-heterocyclyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-heterocyclyl is substituted with halo.

In some embodiments of the compounds of Formula III and stereoisomers and pharmaceutically acceptable salts thereof, one or more of R^{3a1}, R^{3a2}, R^{3a3}, and R^{3a4} is —O-aryl. In some embodiments, R^{3a1} is —O-aryl. In some embodiments, R^{3a2} is —O-aryl. In some embodiments, R^{3a3} is —O-aryl. In some embodiments, R^{3a4} is —O-aryl. In some embodiments, the —O-aryl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-aryl is substituted with halo. In some embodiments, the —O-aryl is substituted with C₁₋₆alkyl. In some embodiments, the —O-aryl is substituted with —C(O)NH₂. In some embodiments, the aryl of is a 6 membered monocyclic aryl. In some embodiments, R^{3a3} is —O-aryl wherein the aryl is an unsubstituted 6-membered monocyclic aryl. In some embodiments, R^{3a1}, R^{3a2}, R^{3a3}, or R^{3a4} is

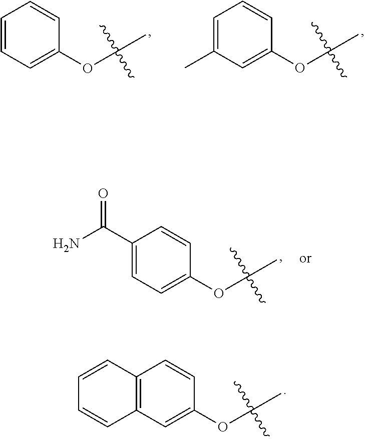

In some embodiments of the compounds of Formula III and stereoisomers and pharmaceutically acceptable salts thereof, one or more of R^{3a1}, R^{3a2}, R^{3a3}, and R^{3a4} is —O-heteroaryl. In some embodiments, R^{3a1} is —O-heteroaryl. In some embodiments, R^{3a2} is —O-heteroaryl. In some embodiments, R^{3a3} is —O-heteroaryl. In some embodiments, R^{3a4} is —O-heteroaryl. In some embodiments, the —O-heteroaryl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-heteroaryl is substituted with halo. In some embodiments, R^{3a1}, R^{3a2}, R^{3a3}, or R^{3a4} is

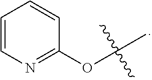 [SMILES: *Oc1ccccn1]

In some embodiments of the compounds of Formula III and stereoisomers and pharmaceutically acceptable salts thereof, one or more of R^{3a1}, R^{3a2}, R^{3a3}, and R^{3a4} is —SO₂-cycloalkyl. In some embodiments, R^{3a1} is —SO-cycloalkyl. In some embodiments, R^{3a2} is —SO₂-cycloalkyl. In some embodiments, R^{3a3} is —SO₂-cycloalkyl. In some embodiments, R^{3a4} is —SO₂-cycloalkyl. In some embodiments, the —SO₂-cycloalkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —SO₂-cycloalkyl is substituted with halo. In some embodiments, R^{3a1}, R^{3a2}, R^{3a3}, or R^{3a4} is

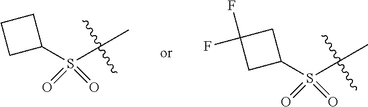 [SMILES: *S(=O)(=O)C1CCC1 ; *S(=O)(=O)C1CC(F)(F)C1]

In some embodiments of the compounds of Formula III and stereoisomers and pharmaceutically acceptable salts thereof, one or more of R^{3a1}, R^{3a2}, R^{3a3}, and R^{3a4} is —NR^{3L1}R^{3L2}. In some embodiments, R^{3a1} is —NR^{3L1}R^{3L2}. In some embodiments, R^{3a2} is —NR^{3L1}R^{3L2}. In some embodiments, R^{3a3} is —NR^{3L1}R^{3L2}. In some embodiments, R^{3a4} is —NR^{3L1}R^{3L2}. In some embodiments, R^{3a1}, R^{3a2}, R^{3a3}, or R^{3a4} is

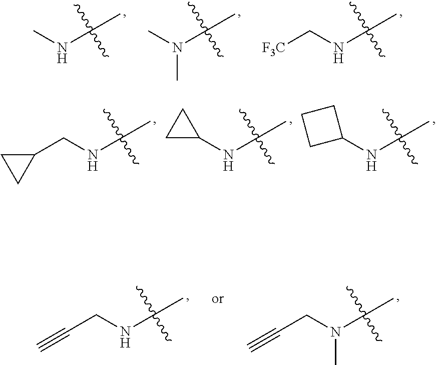

In some embodiments of the compounds of Formula III and stereoisomers and pharmaceutically acceptable salts thereof, R^{3L1} and R^{3L2} are independently H, C₁₋₆alkyl, C₂₋₆alkynyl, or cycloalkyl. In some embodiments, R^{3L1} is H. In some embodiments, R^{3L1} is H and R^{3L2} is C₁₋₆alkyl, C₂₋₆alkynyl, or cycloalkyl. In some embodiments, R^{3L1} is —CH₃. In some embodiments, R^{3L2} is —CH₃ and R^{3L2} is C₁₋₆alkyl, C₂₋₆alkynyl, or cycloalkyl. In some embodiments, R^{3L1} is H or —CH₃ and R^{3L2} is C₁₋₆alkyl. In some embodiments, R^{3L1} is H or —CH₃ and R^{3L2} is C₂₋₆alkynyl. In some embodiments, R^{3L1} is H or —CH₃ and R^{3L2} is cycloalkyl. In some embodiments, the C₁₋₆alkyl, C₂₋₆alkynyl, or cycloalkyl is unsubstituted or substituted with one or more substituents independently selected from the group consisting of halo, C₁₋₆alkyl, C₁₋₆haloalkyl, cycloalkyl, and halocycloalkyl. In some embodiments, the C₁₋₆alkyl is substituted with halo. In some embodiments, the C₁₋₆alkyl is substituted with cycloalkyl.

In some embodiments of the compounds of Formula III and stereoisomers and pharmaceutically acceptable salts thereof, one or more of R^{3a1}, R^{3a2}, R^{3a3}, and R^{3a4} is C₂₋₆alkynyl-NR^{3L3}R^{3L4}. In some embodiments, R^{3a3} is C₂₋₆alkynyl-NR^{3L3}R^{3L4}. In some embodiments, R^{3a2} is C₂₋₆alkynyl-NR^{3L3}R^{3L4}. In some embodiments, R^{3a3} is C₂₋₆alkynyl-NR^{3L3}R^{3L4}. In some embodiments, R^{3a4} is C₂₋₆alkynyl-NR^{3L3}R^{3L4}.

In some embodiments of the compounds of Formula III and stereoisomers and pharmaceutically acceptable salts thereof, R^{3L3} and R^{3L4} are independently H, C₁₋₆alkyl, C₂₋₆alkynyl, or cycloalkyl. In some embodiments, R^{3L3} is H. In some embodiments, R^{3L3} is H and R^{3L4} is C₁₋₆alkyl, C₂₋₆alkynyl, or cycloalkyl. In some embodiments, R^{3L3} is —CH₃. In some embodiments, R^{3L3} is —CH₃ and R^{3L4} is C₁₋₆alkyl, C₂₋₆alkynyl, or cycloalkyl. In some embodiments, R^{3L3} is C₁₋₆alkyl and R^{3L4} is C₁₋₆alkyl. In some embodiments, R^{3L3} is H or —CH₃ and R^{3L4} is C₁₋₆alkyl. In some embodiments, R^{3L3} is H or —CH₃ and R^{3L4} is C₂₋₆alkynyl. In some embodiments, R^{3L3} is H or —CH₃ and R^{3L4} is cycloalkyl. In some embodiments, the C₁₋₆alkyl, C₂₋₆alkynyl, or cycloalkyl is unsubstituted or substituted with one or more substituents independently selected from the group consisting of halo, C₁₋₆alkyl, C₁₋₆haloalkyl, cycloalkyl, and halocycloalkyl. In some embodiments, the C₁₋₆alkyl is substituted with halo. In some embodiments, the C₁₋₆alkyl is substituted with cycloalkyl. In some embodiments, R^{3a1}, R^{3a2}, R^{3a3}, or R^{3a4} is

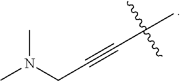 [SMILES: *C#CCN(C)C]

In some embodiments of the compounds of Formula III and stereoisomers and pharmaceutically acceptable salts thereof, two or more of R^{3a1}, R^{3a2}, R^{3a3}, and R^{3a4} is not H. In some embodiments, R^{3a3} is not H and one of R^{3a1}, R^{3a2}, or R^{3a4} is not H. In some embodiments, R^{3a2} is F or Cl and R^{3a3} is —O—C₁₋₆alkyl or —O-cycloalkyl, wherein the —O—C₁₋₆alkyl or —O-cycloalkyl is unsubstituted or substituted with halo or —CN. In some embodiments, R^{3a1} is not H and R^{3a2} is not H. In some embodiments, R^{3a1} is F or Cl and R^{3a2} is F or Cl.

In some embodiments of the compounds of Formula III and stereoisomers and pharmaceutically acceptable salts thereof. X³ is —O—. In some embodiments, X³ is —NR^{3b1}—. In some embodiments, X³ is —NH—. In some embodiments, X³ is —N(CH₃)—. In some embodiments, X³ is —CR^{3b2}R^{3b3}. In some embodiments, X³ is —CH(CH₃)—. In some embodiments, X³ is —CD₂-. In some embodiments, X³ is —CH₂—.

In some embodiments of the compounds of Formula III and stereoisomers and pharmaceutically acceptable salts thereof. X⁴ is —O—. In some embodiments, X⁴ is —NR^{3f2}—. In some embodiments, X⁴ is —NH—. In some embodiments, X⁴ is —N(CH₃)—. In some embodiments, X⁴ is —CR^{3f3}R^{3f4}—. In some embodiments, X⁴ is —CH(CH₃)—. In some embodiments, X⁴ is —CH₂—.

In some embodiments of the compounds of Formula III and stereoisomers and pharmaceutically acceptable salts thereof, B3 is a 3-membered monocyclic heterocyclediyl, a 4-membered monocyclic heterocyclediyl, a 5-membered monocyclic heterocyclediyl comprising 2 or more N, a 6-membered monocyclic heterocyclediyl comprising 2 or more N, a 7-membered monocyclic heterocyclediyl, an 8-membered monocyclic heterocyclediyl, a 7 to 18-membered polycyclic heterocyclediyl, or a 7 to 18-membered spirocyclic heterocyclediyl, wherein the 3-membered monocyclic heterocyclediyl, 4-membered monocyclic heterocyclediyl, 5-membered monocyclic heterocyclediyl, 6-membered monocyclic heterocyclediyl, 7-membered monocyclic heterocyclediyl, 8-membered monocyclic heterocyclediyl, 7 to 18-membered polycyclic heterocyclediyl, or 7 to 18-membered spirocyclic heterocyclediyl of B3 is unsubstituted or substituted with one or more substituents independently selected from the group consisting of halo, C₁₋₆alkyl, and oxo. In some embodiments, the 7 to 18-membered polycyclic heterocyclediyl is a 7, 8, 9, 10, 11, 12, 13, 14, 15, 16, 17, or 18-membered polycyclic heterocyclediyl. In some embodiments, the 7 to 18-membered spirocyclic heterocyclediyl is a 7, 8, 9, 10, 11, 12, 13, 14, 15, 16, 17, or 18-membered spirocyclic heterocyclediyl.

In some embodiments of the compounds of Formula III and stereoisomers and pharmaceutically acceptable salts thereof, B3 is a 3 to 8-membered monocyclic heterocyclediyl, wherein the 3 to 8-membered monocyclic heterocyclediyl is unsubstituted or substituted with one or more substituents selected from the group consisting of halo, C₁₋₆alkyl, and oxo. In some embodiments, the 3 to 8-membered monocyclic heterocyclediyl is a 3, 4, 5, 6, 7, or 8-membered monocyclic heterocyclediyl. In some embodiments, B3 is a 4-membered monocyclic heterocyclediyl, wherein the 4-membered monocyclic heterocyclediyl is unsubstituted or substituted with one or more substituents selected from the group consisting of halo, C₁₋₆alkyl, and oxo. In some embodiments, B3 is

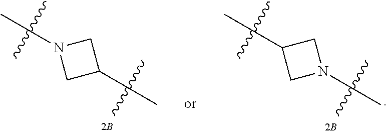 [SMILES: *C1CN(*)C1 ; *C1CN(*)C1]

In some embodiments, B3 is a 6-membered monocyclic heterocyclediyl, wherein the 6-membered monocyclic heterocyclediyl is unsubstituted or substituted with one or more substituents selected from the group consisting of halo, C₁₋₆alkyl, and oxo. In some embodiments, B3 is

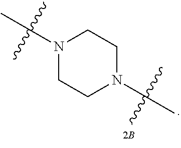 [SMILES: *N1CCN(*)CC1]

In some embodiments, B3 is a 7-membered monocyclic heterocyclediyl, wherein the 7-membered monocyclic heterocyclediyl is unsubstituted or substituted with one or more substituents selected from the group consisting of halo, C₁₋₆alkyl, and oxo. In some embodiments, B3 is

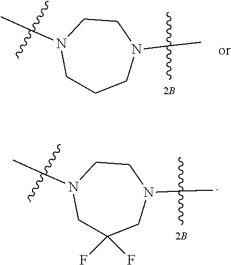 [SMILES: *N1CCCN(*)CC1 ; *N1CCN(*)CC(F)(F)C1]

In some embodiments of the compounds of Formula III and stereoisomers and pharmaceutically acceptable salts thereof, B3 is a 7 to 18-membered polycyclic heterocyclediyl, wherein the 7 to 18-membered polycyclic heterocyclediyl is unsubstituted or substituted with one or more substituents selected from the group consisting of halo, C₁₋₆alkyl, and oxo. In some embodiments, the 7 to 18-membered polycyclic heterocyclediyl is a 7, 8, 9, 10, 11, 12, 13, 14, 15, 16, 17, or 18-membered polycyclic heterocyclediyl. In some embodiments, B3 is

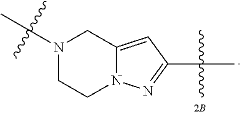 [SMILES: *c1cc2n(n1)CCN(*)C2]

In some embodiments, B3 is

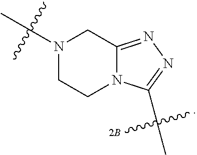 [SMILES: *c1nnc2n1CCN(*)C2]

In some embodiments B3 is

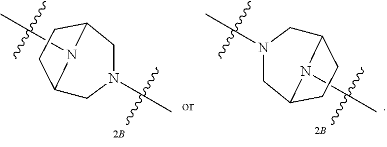 [SMILES: *N1CC2CCC(C1)N2* ; *N1CC2CCC(C1)N2*]

In some embodiments, B3 is

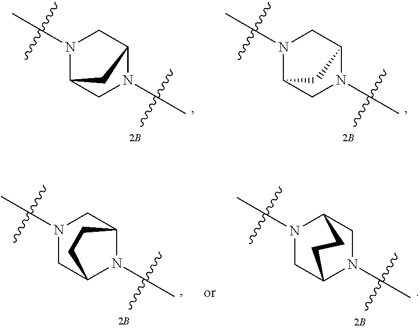

In some embodiments, B3 is

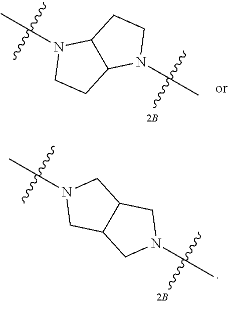 [SMILES: *N1CCC2C1CCN2* ; *N1CC2CN(*)CC2C1]

In some embodiments, B3 is

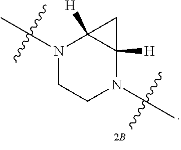 [SMILES: *N1CCN(*)[C@H]2C[C@H]21]

In some embodiments of the compounds of Formula III and stereoisomers and pharmaceutically acceptable salts thereof, B3 is a 7 to 18-membered spirocyclic heterocyclediyl, wherein the 7 to 18-membered spirocyclic heterocyclediyl is unsubstituted or substituted with one or more substituents selected from the group consisting of halo, C₁₋₆alkyl, and oxo. In some embodiments, the 7 to 18-membered spirocyclic heterocyclediyl is a 7, 8, 9, 10, 11, 12, 13, 14, 15, 16, 17, or 18-membered spirocyclic heterocyclediyl. In some embodiments, B3 is

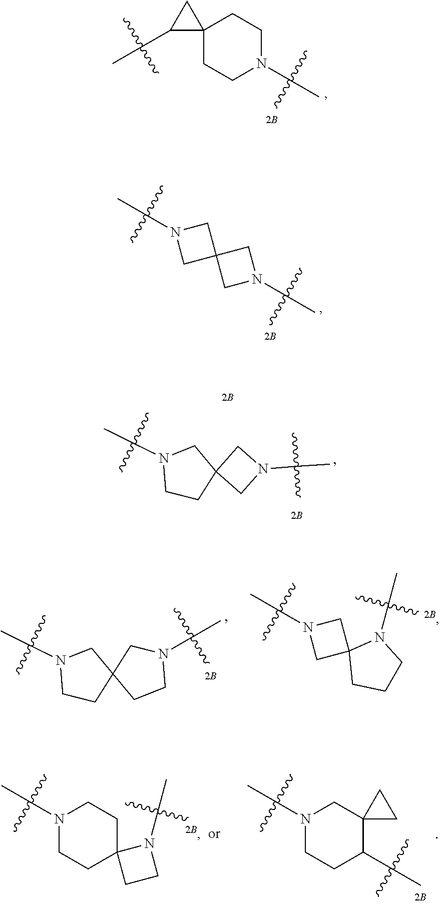

In some embodiments of the compounds of Formula III and stereoisomers and pharmaceutically acceptable salts thereof, the monocyclic heterocyclediyl, polycyclic heterocyclediyl, or spirocyclic heterocyclediyl of B3 comprises one or more N. In some embodiments, the monocyclic heterocyclediyl, polycyclic heterocyclediyl, or spirocyclic heterocyclediyl of B3 comprises two or more N.

In some embodiments of the compounds of Formula III and stereoisomers and pharmaceutically acceptable salts thereof, D3 is C₁₋₆alkyl, wherein the C₁₋₆alkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{3D10})₂. In some embodiments, the C₁₋₆alkyl is unsubstituted or substituted with halo. In some embodiments, the C₁₋₆alkyl is unsubstituted or substituted with —OH. In some embodiments, the C₁₋₆alkyl is unsubstituted or substituted with —O—C₁₋₆alkyl.

In some embodiments of the compounds of Formula III and stereoisomers and pharmaceutically acceptable salts thereof, D3 is cycloalkyl, wherein the cycloalkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{3D10})₂. In some embodiments, the cycloalkyl is unsubstituted or substituted with halo. In some embodiments, D3 is

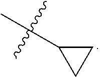 [SMILES: *C1CC1]

In some embodiments of the compounds of formula III and stereoisomers and pharmaceutically acceptable salts thereof, D3 is aryl, wherein the aryl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{3D10})₂. In some embodiments, the aryl is a monocyclic 6-membered aryl. In some embodiments, the aryl is substituted with one or more substituents selected from the group consisting of halo, —CN, C₁₋₆alkyl, and C₁₋₆haloalkyl. In some embodiments, the aryl is substituted with —CN.

In some embodiments of the compounds of Formula III and stereoisomers and pharmaceutically acceptable salts thereof, D3 is heteroaryl, wherein the heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, —C(O)NH₂, —C(O)NHCH₃, and —C(O)N(CH₃)₂.

In some embodiments of the compounds of Formula III and stereoisomers and pharmaceutically acceptable salts thereof, D3 is a monocyclic 5 or 6-membered heteroaryl comprising one or more N, wherein the monocyclic 5 or 6-membered heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, —C(O)NH₂, —C(O)NHCH₃, and —C(O)N(CH₃)₂. In some embodiments, the monocyclic 5 or 6-membered heteroaryl is substituted with —CN. In some embodiments, the monocyclic 5 or 6-membered heteroaryl is substituted with —CF₃. In some embodiments, the monocyclic 5 or 6-membered heteroaryl is substituted with —OCH₃.

In some embodiments of the compounds of Formula III and stereoisomers and pharmaceutically acceptable salts thereof, D3 is —O—C₁₋₆alkyl, wherein the —O—C₁₋₆alkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆ haloalkyl, —C(O)-cycloalkyl, and —C(O)N(R^{3D10})₂. In some embodiments, the —O—C₁₋₆alkyl is unsubstituted or substituted with halo.

In some embodiments of the compounds of Formula III and stereoisomers and pharmaceutically acceptable salts thereof, D3 is —O-aryl or —O-heteroaryl, wherein the —O-aryl or —O-heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{3D10})₂. In some embodiments, the —O-aryl or —O-heteroaryl is unsubstituted or substituted with halo. In some embodiments, the —O-aryl or —O-heteroaryl is unsubstituted or substituted with —CN. In some embodiments, D3 is

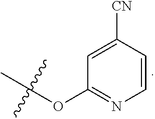 [SMILES: *Oc1cc(C#N)ccn1]

In some embodiments of the compounds of Formula III and stereoisomers and pharmaceutically acceptable salts thereof, D3 is —C(O)—C₁₋₆alkyl, wherein the C(O)—C₁₋₆alkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆ haloalkyl, —C(O)-cycloalkyl, and —C(O)N(R^{3D10})₂. In some embodiments, the —C(O)—C₁₋₆alkyl is unsubstituted or substituted with halo. In some embodiments, the —C(O)—C₁₋₆alkyl is unsubstituted or substituted with —CN. In some embodiments, D3 is

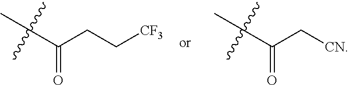 [SMILES: *C(=O)CCC(F)(F)F ; *C(=O)CC#N]

In some embodiments of the compounds of Formula III and stereoisomers and pharmaceutically acceptable salts thereof, D3 is —C(O)-cycloalkyl, wherein the —C(O)-cycloalkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{3D10}). In some embodiments, the —C(O)-cycloalkyl is unsubstituted or substituted with halo. In some embodiments, D3 is

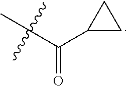 [SMILES: *C(=O)C1CC1]

In some embodiments of the compounds of Formula III and stereoisomers and pharmaceutically acceptable salts thereof, D3 is —C(O)-heterocyclyl, wherein the —C(O)-heterocyclyl is unsubstituted or substituted with one or more substituents selected from the group consisting of halo, —CN, C₁₋₆alkyl, C₁₋₆ haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{3D10})₂. In some embodiments, the —C(O)-heterocyclyl is unsubstituted or substituted with halo. In some embodiments, D3 is

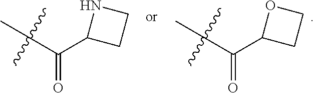 [SMILES: *C(=O)C1CCN1 ; *C(=O)C1CCO1]

In some embodiments of the compounds of Formula III and stereoisomers and pharmaceutically acceptable salts thereof, D3 is —C(O)-aryl or —C(O)-heteroaryl, wherein the —C(O)-aryl or —C(O)-heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{3D10})₂. In some embodiments, the —C(O)-aryl or —C(O)-heteroaryl is unsubstituted or substituted with halo. In some embodiments, the —C(O)-aryl or —C(O)-heteroaryl is unsubstituted or substituted with —CF₃. In some embodiments, the —C(O)-aryl or —C(O)-heteroaryl is unsubstituted or substituted with —CN. In some embodiments, D3 is

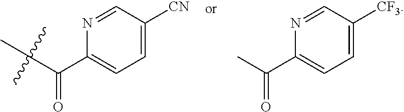

In some embodiments of the compounds of Formula III and stereoisomers and pharmaceutically acceptable salts thereof, D3 is —N(R^{3D1})(R^{3D2}). In some embodiments, D3 is

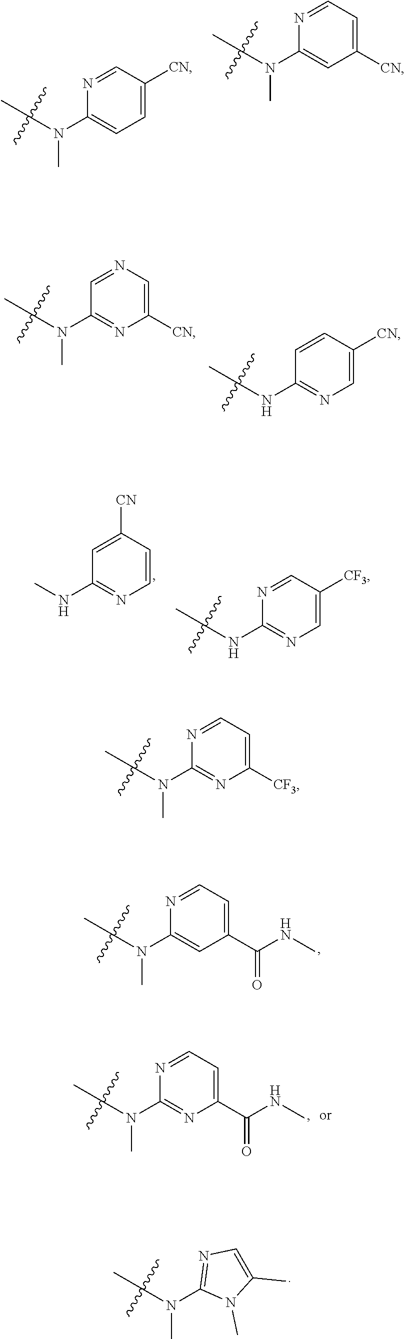

In some embodiments of the compounds of Formula III and stereoisomers and pharmaceutically acceptable salts thereof, R^{3D1} is H. In some embodiments, R^{3D1} is C₁₋₆alkyl. In some embodiments, R^{3D2} is aryl. In some embodiments, R^{3D2} is heteroaryl. In some embodiments, the heteroaryl is a 6-membered heteroaryl.

In some embodiments of the compounds of Formula III and stereoisomers and pharmaceutically acceptable salts thereof, D3 is —C(O)N(R^{3D3})(R^{3D4}). In some embodiments, D3 is

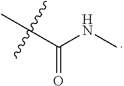 [SMILES: *C(=O)NC]

In some embodiments of the compounds of Formula III and stereoisomers and pharmaceutically acceptable salts thereof, R^{3D3} is H. In some embodiments, R^{3D3} is C₁₋₆alkyl. In some embodiments, R^{3D4} is C₁₋₆alkyl. In some embodiments, R^{3D3} is H and R^{3D4} is C₁₋₆alkyl.

In some embodiments of the compounds of Formula III and stereoisomers and pharmaceutically acceptable salts thereof, D3 is —N(R^{3D5})C(O)R^{3D6}—. In some embodiments, D3 is

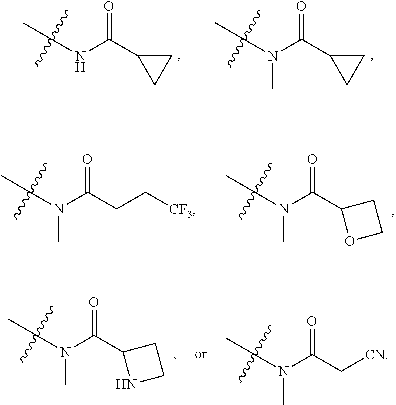

In some embodiments of the compounds of Formula III and stereoisomers and pharmaceutically acceptable salts thereof, R^{3D5} is H. In some embodiments, R^{3D5} is C₁₋₆alkyl. In some embodiments, R^{3D6} is C₁₋₆alkyl. In some embodiments, R^{3D6} is cycloalkyl. In some embodiments, R^{3D6} is heterocyclyl. In some embodiments, R^{3D5} is H and R^{3D6} is C₁₋₆alkyl, cycloalkyl, or heterocyclyl.

In some embodiments, the compound is of Formula IV:

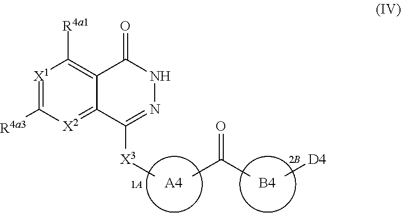

and stereoisomers and pharmaceutically acceptable salts thereof, wherein:
X¹ is —N— or —CR^{4a2}—;
X² is —N— or —CR^{4a4}—;
R^{4a1}, R^{4a2}, R^{4a3}, and R^{4a4} are independently H, halo, —OH, C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, —SO₂-cycloalkyl, —NR^{4L1}R^{4L2}, or C₂₋₆alkynyl-NR^{4L3}R^{4L4};
wherein if X¹ is —CR^{4a2}— and X² is —CR^{4a4}—, then at least one of R^{4a1}, R^{4a2}, R^{4a3}, and R^{4a4} is not H;
wherein each C₁₋₆alkyl, C₂₋₆alkenyl, C₂₋₆alkynyl, cycloalkyl, heterocyclyl, aryl, heteroaryl, —O—C₁₋₆ alkyl, —O—C₂₋₆alkenyl, —O—C₂₋₆alkynyl, —O-cycloalkyl, —O-heterocyclyl, —O-aryl, —O-heteroaryl, or —SO₂-cycloalkyl of R^{4a1}, R^{4a2}, R^{4a3}, and R^{4a4} is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂;
R^{4L1}, R^{4L2}, R^{4L3}, and R^{4L4} are independently H, C₁₋₆alkyl, C₁₋₆alkynyl, or cycloalkyl, wherein each C₁₋₆alkyl, C₂₋₆alkynyl, or cycloalkyl of R^{4L1}, R^{4L2}, R^{4L3}, and R^{4L4} is unsubstituted or substituted with one or more substituents independently selected from the group consisting of halo, C₁₋₆alkyl, C₁₋₆haloalkyl, cycloalkyl, and halocycloalkyl;
X³ is —O—, —NR^{4b1}—, or —CR^{4b2}R^{4b3}—, wherein R^{4b1}, R^{4b2}—, and R^{4b3} are independently H or C₁₋₆alkyl;
A4 is

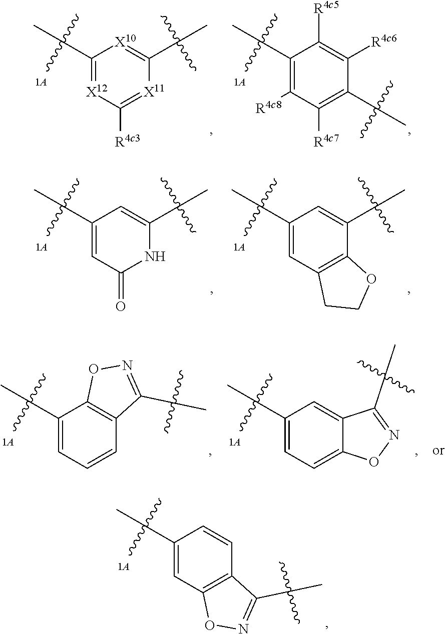

wherein a bond marked 1A is to X³;
X¹⁰ is —N— or —CR^{4c1}—, X¹¹ is —N— or —CR^{4c2}—, and X¹² is —N— or —CR^{4c4}—,
R^{4c1}, R^{4c2}, R^{4c3}, R^{4c4}, R^{4c5}, R^{4c6}, R^{4c7}, and R^{4c8} are independently H, halo, —CN, —SO₂CH₃, —SO₂NH₂, or —NHSO₂CH₃;
B4 is a 3 to 8-membered monocyclic heterocyclediyl, a 7 to 18-membered polycyclic heterocyclediyl, or a 7 to 18-membered spirocyclic heterocyclediyl;
wherein the 3 to 8-membered monocyclic heterocyclediyl, 7 to 18-membered polycyclic heterocyclediyl, or 7 to 18-membered spirocyclic heterocyclediyl of B4 is unsubstituted or substituted with one or more substituents selected from the group consisting of halo, C₁₋₆alkyl, and oxo;
provided that B4 is not

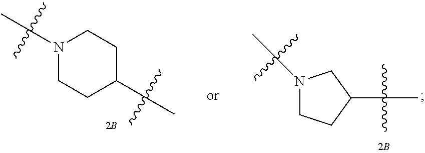 [SMILES: *C1CCN(*)CC1 ; *C1CCN(*)C1]

D4 is C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, —C(O)-heteroaryl, —N(R^{4D1})(R^{4D2}), —C(O)N(R^{4D3})(R^{4D4}), or —N(R^{4D5})C(O)R^{4D6};
wherein the C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, or —C(O)-heteroaryl of D4 is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{4D10})₂, wherein each R^{4D10} is independently H or C₁₋₆alkyl;
R^{4D1}, R^{4D3}, and R^{4D5} are independently H or C₁₋₆alkyl;
R^{4D2} is aryl or heteroaryl, wherein the aryl or heteroaryl of R^{4D2} is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{4D11})₂, wherein each R^{4D11} is independently H or C₁₋₆alkyl; and
R^{4D4} and R^{4D6} are independently C₁₋₆alkyl, cycloalkyl, heterocyclyl, aryl, or heteroaryl, wherein each C₁₋₆alkyl, cycloalkyl, heterocyclyl, aryl, or heteroaryl of R^{4D4} and R^{4D6} is unsubstituted or substituted with one or more substituents independently selected from the group consisting of halo, —CN, C₁₋₆alkyl, C₁₋₆ haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{4D12})₂, wherein each R^{4D12} is independently H or C₁₋₆alkyl;
provided that if B4 is

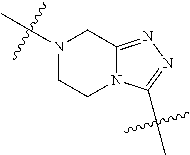 [SMILES: *c1nnc2n1CCN(*)C2]

and D4 is —CHF₂, —CF₂CH₃, or —CF₂CF₃, then R^{4a2} is not F, if X³ is —O—, B4 is

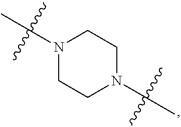 [SMILES: *N1CCN(*)CC1]

and D4 is —C(O)-aryl, then R^{4a4} is not Cl, and if D4 is

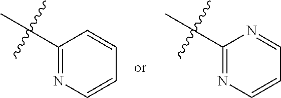 [SMILES: *c1ccccn1 ; *c1ncccn1]

and R^{4c2} is F, then R^{4a2} is not F.

In some embodiments of the compounds of Formula IV and stereoisomers and pharmaceutically acceptable salts thereof, X¹ is —CR^{4a2}—. In some embodiments, X² is —CR^{4a4}—. In some embodiments, X¹ is —N—. In some embodiments, X² is —N—. In some embodiments, X¹ is —CR^{4a2}— and X² is —CR^{4a4}—. In some embodiments. X¹ is —CR^{4a2}—, and X² is —N—. In some embodiments, X¹ is —N— and X² is —CR^{4a4}—.

In some embodiments of the compounds of Formula IV and stereoisomers and pharmaceutically acceptable salts thereof, R^{4a2} is H, Cl, Br, I, —OH, C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, —SO-cycloalkyl, —NR^{4L1}R^{4L2}, or C₂₋₆alkynyl-NR^{4L3}R^{4L4}, wherein each C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, or —SO₂-cycloalkyl of R^{4a2} is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂.

In some embodiments of the compounds of Formula IV and stereoisomers and pharmaceutically acceptable salts thereof, R^{4a4} is H, F, Br, I, —OH, C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, —SO₂-cycloalkyl, —NR^{4L1}R^{4L2}, or C₂₋₆alkynyl-NR^{4L3}R^{4L4}, wherein each C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, or —SO₂-cycloalkyl of R^{4a4} is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂.

In some embodiments of the compounds of Formula IV and stereoisomers and pharmaceutically acceptable salts thereof, one or more of R^{4a1}, R^{4a2}, R^{4a3}, and R^{4a4} is not H. In some embodiments, R^{4a1} is not H. In some embodiments, R^{4a2} is not H. In some embodiments, R^{4a3} is not H. In some embodiments, R^{4a4} is not H.

In some embodiments of the compounds of Formula IV and stereoisomers and pharmaceutically acceptable salts thereof, one or more of R^{4a1}, R^{4a2}, R^{4a3}, and R^{4a4} is halo. In some embodiments, R^{4a1} is halo. In some embodiments, R^{4a2} is halo. In some embodiments, R^{4a3} is halo. In some embodiments, R^{4a4} is halo. In some embodiments, the halo is F, Cl, or Br. In some embodiments, the halo is Cl. In some embodiments, the halo is F. In some embodiments, R^{4a1} is F. In some embodiments, R^{4a3} is F. In some embodiments, R^{4a3} is F.

In some embodiments of the compounds of Formula IV and stereoisomers and pharmaceutically acceptable salts thereof, one or more of R^{4a1}, R^{4a2}, R^{4a3}, and R^{4a4} is —OH. In some embodiments, R^{4a1} is —OH. In some embodiments, R^{4a2} is —OH. In some embodiments, R^{4a3} is —OH. In some embodiments, R^{4a4} is —OH.

In some embodiments of the compounds of Formula IV and stereoisomers and pharmaceutically acceptable salts thereof, one or more of R^{4a1}, R^{4a2}, R^{4a3}, and R^{4a4} is C₁₋₆alkyl. In some embodiments, R^{4a1} is C₁₋₆alkyl. In some embodiments, R^{4a2} is C₁₋₆alkyl. In some embodiments, R^{4a3} is C₁₋₆alkyl. In some embodiments, R^{4a4} is C₁₋₆alkyl. In some embodiments, the C₁₋₆alkyl is unsubstituted or substituted with one or more substituents independently selected from the group consisting of halo, —OH, C₁₋₆alkyl, C₁₋₆ haloalkyl, cycloalkyl, halocycloalkyl, —O—C₁₋₆alkyl, and —C(O)NH₂. In some embodiments, the C₁₋₆alkyl is substituted with halo. In some embodiments, R^{4a1}, R^{4a2}, R^{4a3}, or R^{4a4} is —CH₃, —CH₂CH₃, —CH₂CF₃, —CF₃, —CF₂CH₃, —CH₂CH₂CF₃,

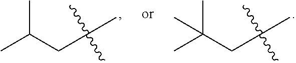

In some embodiments of the compounds of Formula IV and stereoisomers and pharmaceutically acceptable salts thereof, one or more of R^{4a1}, R^{4a2}, R^{4a3}, and R^{4a4} is C₂₋₆alkenyl. In some embodiments, R^{4a1} is C₂₋₆alkenyl. In some embodiments, R^{4a2} is C₂₋₆alkenyl. In some embodiments, R^{4a3} is C₂₋₆alkenyl. In some embodiments, R^{4a4} is C₂₋₆alkenyl. In some embodiments, the C₂₋₆alkenyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the C₂₋₆alkenyl is substituted with halo. In some embodiments, R^{4a1}, R^{4a2}, R^{4a3}, or R^{4a4} is

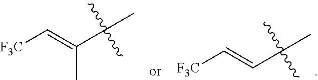

In some embodiments of the compounds of Formula IV and stereoisomers and pharmaceutically acceptable salts thereof, one or more of R^{4a1}, R^{4a2}, R^{4a3}, and R^{4a4} is C₂₋₆alkynyl. In some embodiments, R^{4a1} is C₂₋₆alkynyl. In some embodiments, R^{4a2} is C₂₋₆alkynyl. In some embodiments, R^{4a3} is C₂₋₆alkynyl. In some embodiments, R^{4a4} is C₂₋₆alkynyl. In some embodiments, the C₂₋₆alkynyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the C₂₋₆alkynyl is substituted with halo. In some embodiments, the C₂₋₆alkynyl is substituted with cycloalkyl. In some embodiments, R^{4a1}, R^{4a2}, R^{4a3}, or R^{4a4} is

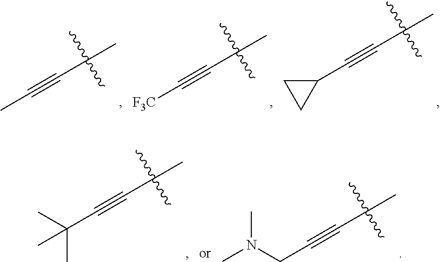

In some embodiments of the compounds of Formula IV and stereoisomers and pharmaceutically acceptable salts thereof, one or more of R^{4a1}, R^{4a2}, R^{4a3}, and R^{4a4} is cycloalkyl. In some embodiments, R^{4a1} is cycloalkyl. In some embodiments, R^{4a2} is cycloalkyl. In some embodiments, R^{4a3} is cycloalkyl. In some embodiments, R^{4a4} is cycloalkyl. In some embodiments, the cycloalkyl is unsubstituted or substituted with one or more substituents independently selected from the group consisting of halo, —OH, C₁₋₆alkyl, C₁₋₆ haloalkyl, cycloalkyl, halocycloalkyl, —O—C₁₋₆alkyl, and —C(O)NH₂. In some embodiments, the cycloalkyl is substituted with halo. In some embodiments, R^{4a1}, R^{4a2}, R^{4a3}, or R^{4a4} is

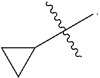 [SMILES: *C1CC1]

In some embodiments of the compounds of Formula IV and stereoisomers and pharmaceutically acceptable salts thereof, one or more of R^{4a1}, R^{4a2}, R^{4a3}, and R^{4a4} is heterocyclyl. In some embodiments, R^{4a1} is heterocyclyl. In some embodiments, R^{4a2} is heterocyclyl. In some embodiments, R^{4a3} is heterocyclyl. In some embodiments, R^{4a4} is heterocyclyl. In some embodiments, the heterocyclyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆ alkyl, and —C(O)NH₂. In some embodiments, the heterocyclyl is substituted with halo. In some embodiments, the heterocyclyl is substituted with —OH. In some embodiments, R^{4a1}, R^{4a2}, R^{4a3}, or R^{4a4} is

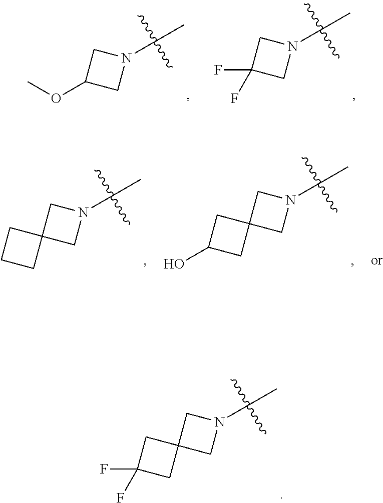

In some embodiments of the compounds of Formula IV and stereoisomers and pharmaceutically acceptable salts thereof, one or more of R^{4a1}, R^{4a2}, R^{4a3}, and R^{4a4} is aryl or heteroaryl. In some embodiments, R^{4a1} is aryl or heteroaryl. In some embodiments, R^{4a2} is aryl or heteroaryl. In some embodiments, R^{4a3} is aryl or heteroaryl. In some embodiments, R^{4a4} is aryl or heteroaryl. In some embodiments, the aryl or heteroaryl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, R^{4a1}, R^{4a2}, R^{4a3}, or R^{4a4} is

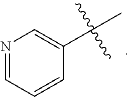 [SMILES: *c1cccnc1]

In some embodiments of the compounds of Formula IV and stereoisomers and pharmaceutically acceptable salts thereof, one or more of R^{4a1}, R^{4a2}, R^{4a3}, and R^{4a4} is —O—C₁₋₆alkyl. In some embodiments, R^{4a1} is —O—C₁₋₆alkyl. In some embodiments, R^{4a2} is —O—C₁₋₆alkyl. In some embodiments, R^{4a3} is —O—C₁₋₆alkyl. In some embodiments, R^{4a4} is —O—C₁₋₆alkyl. In some embodiments, the —O—C₁₋₆alkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O—C₁₋₆alkyl is substituted with halo. In some embodiments, the —O—C₁₋₆alkyl is substituted with cycloalkyl. In some embodiments, R^{4a1}, R^{4a2}, R^{4a3}, or R^{4a4} is —OCH₃, —OCHF₂, —OCH₂CF₃, or

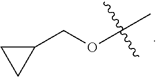 [SMILES: *OCC1CC1]

In some embodiments of the compounds of Formula IV and stereoisomers and pharmaceutically acceptable salts thereof, one or more of R^{4a1}, R^{4a2}, R^{4a3}, and R^{4a4} is —O—C₂₋₆alkenyl. In some embodiments, R^{4a1} is —O—C₂₋₆alkenyl. In some embodiments, R^{4a2} is —O—C₂₋₆alkenyl. In some embodiments, R^{4a3} is —O—C₂₋₆ alkenyl. In some embodiments, R^{4a4} is —O—C₂₋₆alkenyl. In some embodiments, the —O—C₂₋₆alkenyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the O—C₂₋₆alkenyl is substituted with halo.

In some embodiments of the compounds of Formula IV and stereoisomers and pharmaceutically acceptable salts thereof, one or more of R^{4a1}, R^{4a2}, R^{4a3}, and R^{4a4} is —O—C₂₋₆alkynyl. In some embodiments, R^{4a1} is —O—C₂₋₆alkynyl. In some embodiments, R^{4a2} is —O—C₂₋₆alkynyl. In some embodiments, R^{4a3} is —O—C₂₋₆alkynyl. In some embodiments, R^{4a4} is —O—C₂₋₆alkynyl. In some embodiments, the —O—C₂₋₆alkynyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O—C₂₋₆alkynyl is substituted with halo. In some embodiments, R^{4a1}, R^{4a2}, R^{4a3}, or R^{4a4} is

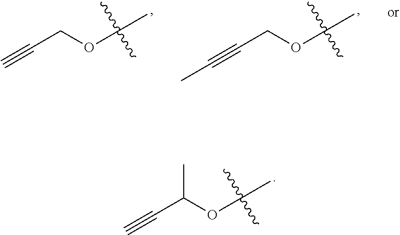

In some embodiments of the compounds of Formula IV and stereoisomers and pharmaceutically acceptable salts thereof, one or more of R^{4a1}, R^{4a2}, R^{4a3}, and R^{4a4} is —O-cycloalkyl. In some embodiments, R^{4a1} is —O-cycloalkyl. In some embodiments, R^{4a2} is —O-cycloalkyl. In some embodiments, R^{4a3} is —O-cycloalkyl. In some embodiments, R^{4a4} is cycloalkyl. In some embodiments, the —O-cycloalkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-cycloalkyl is substituted with halo. In some embodiments, the —O-cycloalkyl is substituted with C₁₋₆haloalkyl. In some embodiments, R^{4a1}, R^{4a2}, R^{4a3}, or R^{4a4} is

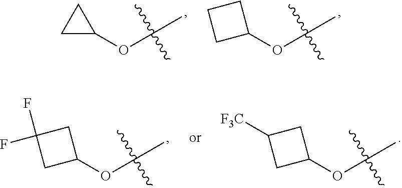 [SMILES: *OC1CC1 ; *OC1CCC1 ; *OC1CC(F)(F)C1 ; *OC1CC(C(F)(F)F)C1]

In some embodiments of the compounds of Formula IV and stereoisomers and pharmaceutically acceptable salts thereof, one or more of R^{4a1}, R^{4a2}, R^{4a3}, and R^{4a4} is —O-heterocyclyl. In some embodiments, R^{4a1} is —O-heterocyclyl. In some embodiments, R^{4a2} is —O-heterocyclyl. In some embodiments, R^{4a3} is —O-heterocyclyl. In some embodiments, R^{4a4} is —O-heterocyclyl. In some embodiments, the —O-heterocyclyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-heterocyclyl is substituted with halo.

In some embodiments of the compounds of Formula IV and stereoisomers and pharmaceutically acceptable salts thereof, one or more of R^{4a1}, R^{4a2}, R^{4a3}, and R^{4a4} is —O-aryl. In some embodiments, R^{4a1} is —O-aryl. In some embodiments, R^{4a2} is —O-aryl. In some embodiments, R^{4a3} is —O-aryl. In some embodiments, R^{4a4} is —O-aryl. In some embodiments, the —O-aryl is unsubstituted or substituted with one or more substituents independently selected from the group consisting of halo, —OH, C₁₋₆alkyl, C₁₋₆ haloalkyl, cycloalkyl, halocycloalkyl, —O—C₁₋₆alkyl, and —C(O)NH₂. In some embodiments, the —O-aryl is substituted with halo. In some embodiments, the —O-aryl is substituted with C₁₋₆alkyl. In some embodiments, the —O-aryl is substituted with —C(O)NH₂. In some embodiments, the aryl of is a 6 membered monocyclic aryl. In some embodiments, R^{4a3} is —O-aryl wherein the aryl is an unsubstituted 6-membered monocyclic aryl. In some embodiments, R^{4a1}, R^{4a2}, R^{4a3}, or R^{4a4} is

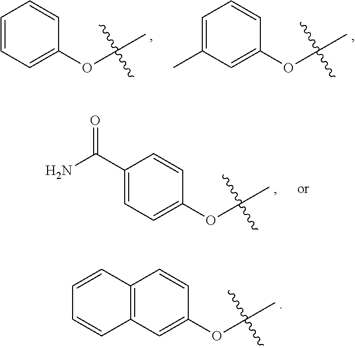

In some embodiments of the compounds of Formula IV and stereoisomers and pharmaceutically acceptable salts thereof, one or more of R^{4a1}, R^{4a2}, R^{4a3}, and R^{4a4} is —O-heteroaryl. In some embodiments, R^{4a1} is —O-heteroaryl. In some embodiments, R^{4a2} is —O-heteroaryl. In some embodiments, R^{4a3} is —O-heteroaryl. In some embodiments, R^{4a4} is —O-heteroaryl. In some embodiments, the —O-heteroaryl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-heteroaryl is substituted with halo. In some embodiments, R^{4a1}, R^{4a2}, R^{4a3}, or R^{4a4}, is

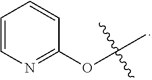 [SMILES: *Oc1ccccn1]

In some embodiments of the compounds of Formula IV and stereoisomers and pharmaceutically acceptable salts thereof, one or more of R^{4a1}, R^{4a2}, R^{4a3}, and R^{4a4} is —SO₂-cycloalkyl. In some embodiments, R^{4a1} is —SO₂-cycloalkyl. In some embodiments, R^{4a2} is —SO₂-cycloalkyl. In some embodiments, R^{4a3} is —SO₂-cycloalkyl. In some embodiments, R^{4a4} is —SO₂-cycloalkyl. In some embodiments, the —SO₂-cycloalkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —SO₂-cycloalkyl is substituted with halo. In some embodiments, R^{4a1}, R^{4a2}, R^{4a3}, or R^{4a4} is

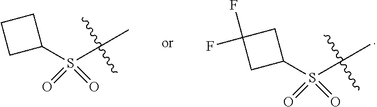 [SMILES: *S(=O)(=O)C1CCC1 ; *S(=O)(=O)C1CC(F)(F)C1]

In some embodiments of the compounds of Formula IV and stereoisomers and pharmaceutically acceptable salts thereof, one or more of R^{4a1}, R^{4a2}, R^{4a3}, and R^{4a4} is —NR^{4L1}R^{4L2}. In some embodiments, R^{4a1} is —NR^{4L1}R^{4L2}. In some embodiments, R^{4a2} is —NR^{4L1}R^{4L2}. In some embodiments, R^{4a3} is —NR^{4L1}R^{4L2}. In some embodiments, R^{4a4} is —NR^{4L1}R^{4L2}. In some embodiments, R^{4a1}, R^{4a2}, R^{4a3}, or R^{4a4} is

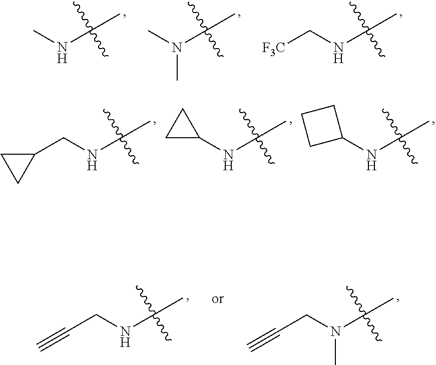

In some embodiments of the compounds of Formula IV and stereoisomers and pharmaceutically acceptable salts thereof, R^{4L1} and R^{4L2} are independently H, C₁₋₆alkyl, C₂₋₆alkynyl, or cycloalkyl. In some embodiments, R^{4L1} is H. In some embodiments, R^{4L1} is H and R^{4L2} is C₁₋₆alkyl, C₂₋₆alkynyl, or cycloalkyl. In some embodiments, R^{4L1} is —CH₃. In some embodiments, R^{4L1} is —CH₃ and R^{4L2} is C₁₋₆alkyl, C₂₋₆alkynyl, or cycloalkyl. In some embodiments, R^{4L1} is H or —CH₃ and R^{4L2} is C₁₋₆alkyl. In some embodiments, R^{4L1} is H or —CH₃ and R^{4L2} is C₂₋₆alkynyl. In some embodiments, R^{4L1} is H or —CH₃ and R^{4L2} is cycloalkyl. In some embodiments, the C₁₋₆ alkyl, C₂₋₆alkynyl, or cycloalkyl is unsubstituted or substituted with one or more substituents independently selected from the group consisting of halo, C₁₋₆alkyl, C₁₋₆haloalkyl, cycloalkyl, and halocycloalkyl. In some embodiments, the C₁₋₆alkyl is substituted with halo. In some embodiments, the C₁₋₆alkyl is substituted with cycloalkyl.

In some embodiments of the compounds of Formula IV and stereoisomers and pharmaceutically acceptable salts thereof, one or more of R^{4a1}, R^{4a2}, R^{4a3}, and R^{4a4} is C₂₋₆alkynyl-NR^{4L3}R^{4L4}. In some embodiments, R^{4a1} is C₂₋₆alkynyl-NR^{4L3}R^{4L4}. In some embodiments, R^{4a2} is C₂₋₆alkynyl-NR^{4L3}R^{4L4}. In some embodiments, R^{4a3} is C₂₋₆-alkynyl-NR^{4L3}R^{4L4}. In some embodiments, R^{4a2} is C₂₋₆alkynyl-NR^{4L3}R^{4L4}.

In some embodiments of the compounds of Formula IV and stereoisomers and pharmaceutically acceptable salts thereof, R^{4L3} and R^{4L4} are independently H, C₁₋₆alkyl, C₂₋₆alkynyl, or cycloalkyl. In some embodiments, R^{4L3} is H. In some embodiments, R^{4L3} is H and R^{4L4} is C₁₋₆alkyl, C₂₋₆-alkynyl, or cycloalkyl. In some embodiments, R^{4L3} is —CH₃. In some embodiments, R^{4L3} is —CH₃ and R^{4L4} is C₁₋₆alkyl, C₂₋₆alkynyl, or cycloalkyl. In some embodiments, R^{4L3} is C₁₋₆alkyl and R^{4L4} is C₁₋₆alkyl. In some embodiments, R^{4L3} is H or —CH₃ and R^{4L4} is C₁₋₆alkyl. In some embodiments, R^{4L3} is H or —CH₃ and R^{4L4} is C₂₋₆alkynyl. In some embodiments, R^{4L3} is H or —CH₃ and R^{4L4} is cycloalkyl. In some embodiments, the C₁₋₆alkyl, C₂₋₆alkynyl, or cycloalkyl is unsubstituted or substituted with one or more substituents independently selected from the group consisting of halo, C₁₋₆alkyl, C₁₋₆haloalkyl, cycloalkyl, and halocycloalkyl. In some embodiments, the C₁₋₆alkyl is substituted with halo. In some embodiments, the C₁₋₆alkyl is substituted with cycloalkyl. In some embodiments, R^{4a1}, R^{4a2}, R^{4a3}, or R^{4a4} is

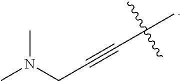 [SMILES: *C#CCN(C)C]

In some embodiments of the compounds of Formula IV and stereoisomers and pharmaceutically acceptable salts thereof, two or more of R^{4a1}, R^{4a2}, R^{4a3}, and R^{4a4} is not H. In some embodiments, R^{4a3} is not H and one of R^{4a1}, R^{4a2}, or R^{4a4} is not H. In some embodiments, R^{4a2} is F or Cl and R^{4a3} is —O—C₁₋₆alkyl or —O-cycloalkyl, wherein the —O—C₁₋₆alkyl or —O-cycloalkyl is unsubstituted or substituted with halo or —CN. In some embodiments, R^{4a1} is not H and R^{4a2} is not H. In some embodiments, R^{4a1} is F or Cl and R^{4a2} is F or Cl.

In some embodiments of the compounds of Formula IV and stereoisomers and pharmaceutically acceptable salts thereof. X is —O—. In some embodiments, X³ is —NR^{4b1}—. In some embodiments, X³ is —NH—. In some embodiments, X³ is —N(CH₃)—. In some embodiments, X³ is —CR^{4b2}R^{4b3}—. In some embodiments, X³ is —CH(CH₃)—. In some embodiments, X³ is —CD₂-. In some embodiments, X³ is —CH₂—.

In some embodiments of the compounds of Formula IV and stereoisomers and pharmaceutically acceptable salts thereof, A4 is

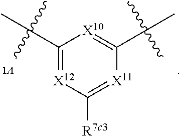

In some embodiments, one or more of X¹⁰, X¹¹, and X¹² is —N—. In some embodiments, X¹⁰ is —N—. In some embodiments, X¹¹ is —N—. In some embodiments, X¹² is —N—. In some embodiments, X¹⁰ is —CR^{4c1}—. In some embodiments, X¹¹ is —CR^{4c2}—. In some embodiments, X¹² is —CR^{4c3}—. In some embodiments, X¹⁰ is —CR^{4c1}—, X¹¹ is —CR^{4c2}—, and X¹² is —CR^{4c3}—.

In some embodiments of the compounds of Formula IV and stereoisomers and pharmaceutically acceptable salts thereof, one or more of R^{4c1}, R^{4c2}, R^{4c3}, and R^{4c4} is not H. In some embodiments, one or more of R^{4c1}, R^{4c2}, R^{4c3}, and R^{4c4} is —CN. In some embodiments, one or more of R^{4c1}, R^{4c2}, R^{4c3}, and R^{4c4} is independently Cl, F, or Br. In some embodiments, two or more of R^{4c1}, R^{4c2}, R^{4c3}, and R^{4c4} are independently Cl, F, or Br. In some embodiments, R^{4c2} is F and R^{1c1}, R^{1c3}, and R^{4c1}, R^{4c3}, and R^{4c4} are H. In some embodiments, R^{4c1}, R^{4c2}, R^{4c3}, and R^{4c4}, are H.

In some embodiments of the compounds of Formula IV and stereoisomers and pharmaceutically acceptable salts thereof, A4 is

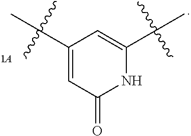 [SMILES: "." ; *c1cc(*)[nH]c(=O)c1]

In some embodiments, A4 is

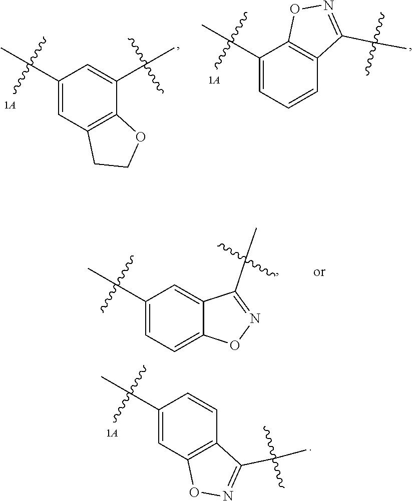

In some embodiments, A4 is

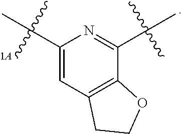 [SMILES: *c1cc2c(c(*)n1)OCC2]

In some embodiments, A4 is

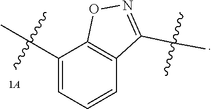 [SMILES: *c1noc2c(*)cccc12]

In some embodiments, A4 is

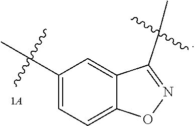 [SMILES: *c1ccc2onc(*)c2c1]

In some embodiments, A4 is

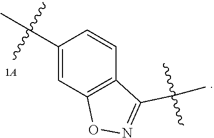 [SMILES: *c1ccc2c(*)noc2c1]

In some embodiments of the compounds of Formula IV and stereoisomers and pharmaceutically acceptable salts thereof, B4 is a 3-membered monocyclic heterocyclediyl, a 4-membered monocyclic heterocyclediyl, a 5-membered monocyclic heterocyclediyl comprising 2 or more N, a 6-membered monocyclic heterocyclediyl comprising 2 or more N, a 7-membered monocyclic heterocyclediyl, an 8-membered monocyclic heterocyclediyl, a 7 to 18-membered polycyclic heterocyclediyl, or a 7 to 18-membered spirocyclic heterocyclediyl, wherein the 3-membered monocyclic heterocyclediyl, 4-membered monocyclic heterocyclediyl, 5-membered monocyclic heterocyclediyl, 6-membered monocyclic heterocyclediyl, 7-membered monocyclic heterocyclediyl, 8-membered monocyclic heterocyclediyl, 7 to 18-membered polycyclic heterocyclediyl, or 7 to 18-membered spirocyclic heterocyclediyl of B4 is unsubstituted or substituted with one or more substituents independently selected from the group consisting of halo, C₁₋₆alkyl, and oxo. In some embodiments, the 7 to 18-membered polycyclic heterocyclediyl is a 7, 8, 9, 10, 11, 12, 13, 14, 15, 16, 17, or 18-membered polycyclic heterocyclediyl. In some embodiments, the 7 to 18-membered spirocyclic heterocyclediyl is a 7, 8, 9, 10, 11, 12, 13, 14, 15, 16, 17, or 18-membered spirocyclic heterocyclediyl.

In some embodiments of the compounds of Formula IV and stereoisomers and pharmaceutically acceptable salts thereof, B4 is a 3 to 8-membered monocyclic heterocyclediyl, wherein the 3 to 8-membered monocyclic heterocyclediyl is unsubstituted or substituted with one or more substituents selected from the group consisting of halo, C₁₋₆alkyl, and oxo. In some embodiments, the 3 to 8-membered monocyclic heterocyclediyl is a 3, 4, 5, 6, 7, or 8-membered monocyclic heterocyclediyl. In some embodiments, B4 is a 4-membered monocyclic heterocyclediyl, wherein the 4-membered monocyclic heterocyclediyl is unsubstituted or substituted with one or more substituents selected from the group consisting of halo, C₁₋₆alkyl, and oxo. In some embodiments, B4 is

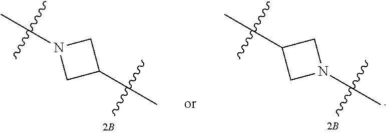 [SMILES: *C1CN(*)C1 ; *C1CN(*)C1]

In some embodiments, B4 is a 6-membered monocyclic heterocyclediyl, wherein the 6-membered monocyclic heterocyclediyl is unsubstituted or substituted with one or more substituents selected from the group consisting of halo, C₁₋₆alkyl, and oxo. In some embodiments, B4 is

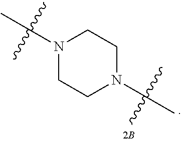 [SMILES: *N1CCN(*)CC1]

In some embodiments, B4 is a 7-membered monocyclic heterocyclediyl, wherein the 7-membered monocyclic heterocyclediyl is unsubstituted or substituted with one or more substituents selected from the group consisting of halo, C₁₋₆alkyl, and oxo. In some embodiments, B4 is

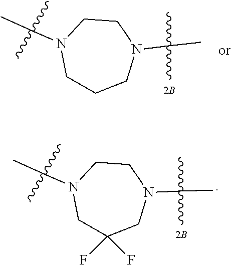 [SMILES: *N1CCCN(*)CC1 ; *N1CCN(*)CC(F)(F)C1]

In some embodiments of the compounds of Formula IV and stereoisomers and pharmaceutically acceptable salts thereof, B4 is a 7 to 18-membered polycyclic heterocyclediyl, wherein the 7 to 18-membered polycyclic heterocyclediyl is unsubstituted or substituted with one or more substituents selected from the group consisting of halo, C₁₋₆alkyl, and oxo. In some embodiments, the 7 to 18-membered polycyclic heterocyclediyl is a 7, 8, 9, 10, 11, 12, 13, 14, 15, 16, 17, or 18-membered polycyclic heterocyclediyl. In some embodiments, B4 is

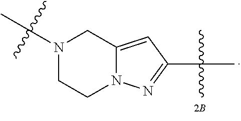 [SMILES: *c1cc2n(n1)CCN(*)C2]

In some embodiments, B4 is

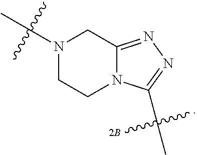 [SMILES: *c1nnc2n1CCN(*)C2]

In some embodiments B4 is

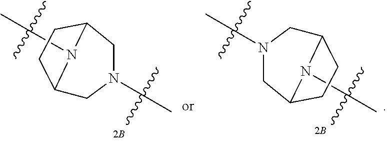 [SMILES: *N1CC2CCC(C1)N2* ; *N1CC2CCC(C1)N2*]

In some embodiments, B4 is

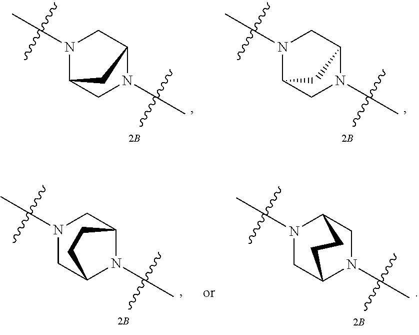

In some embodiments, B4 is

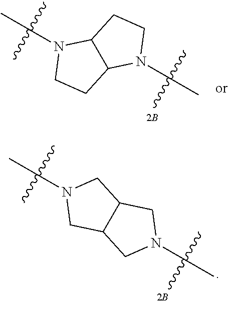 [SMILES: *N1CCC2C1CCN2* ; *N1CC2CN(*)CC2C1]

In some embodiments, B4 is

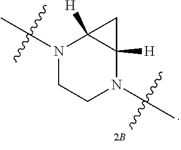 [SMILES: *N1CCN(*)[C@H]2C[C@H]21]

In some embodiments of the compounds of Formula IV and stereoisomers and pharmaceutically acceptable salts thereof, B4 is a 7 to 18-membered spirocyclic heterocyclediyl, wherein the 7 to 18-membered spirocyclic heterocyclediyl is unsubstituted or substituted with one or more substituents selected from the group consisting of halo, C₁₋₆alkyl, and oxo. In some embodiments, the 7 to 18-membered spirocyclic heterocyclediyl is a 7, 8, 9, 10, 11, 12, 13, 14, 15, 16, 17, or 18-membered spirocyclic heterocyclediyl. In some embodiments, B4 is

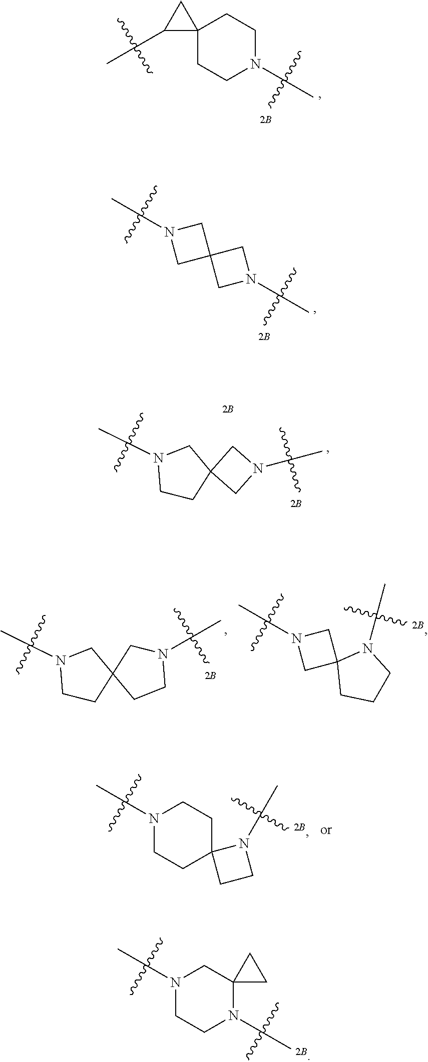

In some embodiments of the compounds of Formula IV and stereoisomers and pharmaceutically acceptable salts thereof, the monocyclic heterocyclediyl, polycyclic heterocyclediyl, or spirocyclic heterocyclediyl of B4 comprises one or more N. In some embodiments, the monocyclic heterocyclediyl, polycyclic heterocyclediyl, or spirocyclic heterocyclediyl of B4 comprises two or more N.

In some embodiments of the compounds of Formula IV and stereoisomers and pharmaceutically acceptable salts thereof, D4 is C₁₋₆alkyl, wherein the C₁₋₆alkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{4D10})₂. In some embodiments, the C₁₋₆ alkyl is unsubstituted or substituted with halo. In some embodiments, the C₁₋₆alkyl is unsubstituted or substituted with —OH. In some embodiments, the C₁₋₆alkyl is unsubstituted or substituted with —O—C₁₋₆alkyl.

In some embodiments of the compounds of Formula IV and stereoisomers and pharmaceutically acceptable salts thereof, D4 is cycloalkyl, wherein the cycloalkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{4D10})₂. In some embodiments, the cycloalkyl is unsubstituted or substituted with halo. In some embodiments, D4 is

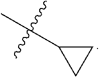 [SMILES: *C1CC1]

In some embodiments of the compounds of Formula IV and stereoisomers and pharmaceutically acceptable salts thereof, D4 is aryl, wherein the aryl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{4D10})₂. In some embodiments, the aryl is a monocyclic 6-membered aryl. In some embodiments, the aryl is substituted with one or more substituents selected from the group consisting of halo, —CN, C₁₋₆alkyl, and C₁₋₆haloalkyl. In some embodiments, the aryl is substituted with —CN.

In some embodiments of the compounds of Formula IV and stereoisomers and pharmaceutically acceptable salts thereof, D4 is heteroaryl, wherein the heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, —C(O)NH₂, —C(O)NHCH₃, and —C(O)N(CH₃)₂.

In some embodiments of the compounds of Formula IV and stereoisomers and pharmaceutically acceptable salts thereof, D4 is a monocyclic 5 or 6-membered heteroaryl comprising one or more N, wherein the monocyclic 5 or 6-membered heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, —C(O)NH₂, —C(O)NHCH₃, and —C(O)N(CH₃)₂. In some embodiments, the monocyclic 5 or 6-membered heteroaryl is substituted with —CN. In some embodiments, the monocyclic 5 or 6-membered heteroaryl is substituted with —CF₃. In some embodiments, the monocyclic 5 or 6-membered heteroaryl is substituted with —OCH₃.

In some embodiments of the compounds of Formula IV and stereoisomers and pharmaceutically acceptable salts thereof, D4 is —O—C₁₋₆alkyl, wherein the —O—C₁₋₆alkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{4D10})₂. In some embodiments, the —O—C₁₋₆alkyl is unsubstituted or substituted with halo.

In some embodiments of the compounds of Formula IV and stereoisomers and pharmaceutically acceptable salts thereof, D4 is —O-aryl or —O-heteroaryl, wherein the —O-aryl or —O-heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R′)₂. In some embodiments, the —O-aryl or —O-heteroaryl is unsubstituted or substituted with halo. In some embodiments, the —O-aryl or —O-heteroaryl is unsubstituted or substituted with —CN. In some embodiments, D4 is

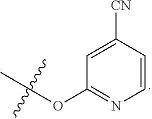 [SMILES: *Oc1cc(C#N)ccn1 ; "."]

In some embodiments of the compounds of Formula IV and stereoisomers and pharmaceutically acceptable salts thereof, D4 is —C(O)—C₁₋₆alkyl, wherein the —C(O)—C₁₋₆alkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆ haloalkyl, —C(O)-cycloalkyl, and —C(O)N(R^{4D10})₂. In some embodiments, the —C(O)—C₁₋₆alkyl is unsubstituted or substituted with halo. In some embodiments, the —C(O)—C₁₋₆alkyl is unsubstituted or substituted with —CN. In some embodiments, D4 is

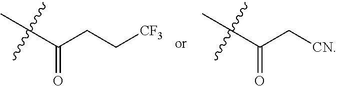 [SMILES: *C(=O)CCC(F)(F)F ; *C(=O)CC#N]

In some embodiments of the compounds of Formula IV and stereoisomers and pharmaceutically acceptable salts thereof, D4 is —C(O)-cycloalkyl, wherein the —C(O)-cycloalkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{4D10}). In some embodiments, the —C(O)-cycloalkyl is unsubstituted or substituted with halo. In some embodiments, D4 is

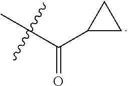 [SMILES: *C(=O)C1CC1]

In some embodiments of the compounds of Formula IV and stereoisomers and pharmaceutically acceptable salts thereof, D4 is —C(O)-heterocyclyl, wherein the —C(O)-heterocyclyl is unsubstituted or substituted with one or more substituents selected from the group consisting of halo, —CN, C₁₋₆alkyl, C₁₋₆ haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{4D10})₂. In some embodiments, the —C(O)-heterocyclyl is unsubstituted or substituted with halo. In some embodiments, D4 is

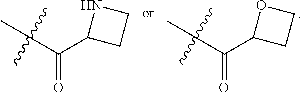 [SMILES: *C(=O)C1CCN1 ; "or" ; *C(=O)C1CCO1 ; "."]

In some embodiments of the compounds of Formula IV and stereoisomers and pharmaceutically acceptable salts thereof, D4 is —C(O)-aryl or —C(O)-heteroaryl, wherein the —C(O)-aryl or —C(O)-heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{4D10}). In some embodiments, the —C(O)-aryl or —C(O)-heteroaryl is unsubstituted or substituted with halo. In some embodiments, the —C(O)-aryl or —C(O)-heteroaryl is unsubstituted or substituted with —CF₃. In some embodiments, the —C(O)-aryl or —C(O)-heteroaryl is unsubstituted or substituted with —CN. In some embodiments, D4 is

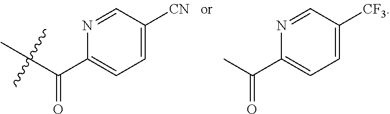

In some embodiments of the compounds of Formula IV and stereoisomers and pharmaceutically acceptable salts thereof, D4 is —N(R^{4D1})(R^{4D2}). In some embodiments, D4 is

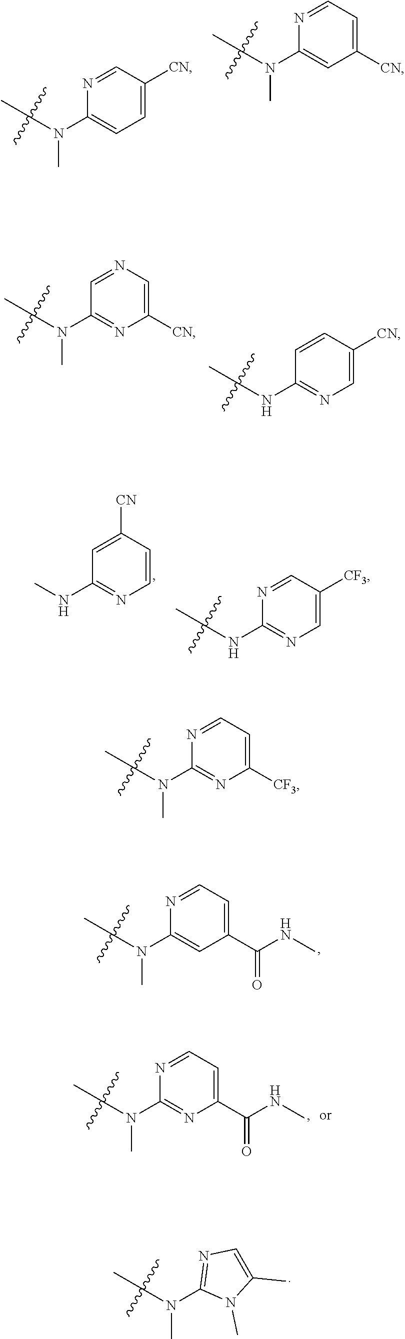

In some embodiments of the compounds of Formula IV and stereoisomers and pharmaceutically acceptable salts thereof, R^{4D1} is H. In some embodiments, R^{4D1} is C₁₋₆alkyl. In some embodiments, R^{4D2} is aryl. In some embodiments, R^{4D2} is heteroaryl. In some embodiments, the heteroaryl is a 6-membered heteroaryl.

In some embodiments of the compounds of Formula IV and stereoisomer and pharmaceutically acceptable salts thereof, D4 is —C(O)N(R^{4D3})(R^{4D4}). In some embodiments, D4 is

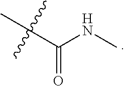

In some embodiments of the compounds of Formula IV and stereoisomers and pharmaceutically acceptable salts thereof, R^{4D3} is H. In some embodiments, R^{4D3} is C₁₋₆alkyl. In some embodiments, R^{4D4} is C₁₋₆alkyl. In some embodiments, R^{4D3} is H and R^{4D4} is C₁₋₆alkyl.

In some embodiments of the compounds of Formula IV and stereoisomers and pharmaceutically acceptable salts thereof, D4 is —N(R^{4D5})C(O)R^{4D6}. In some embodiments, D4 is

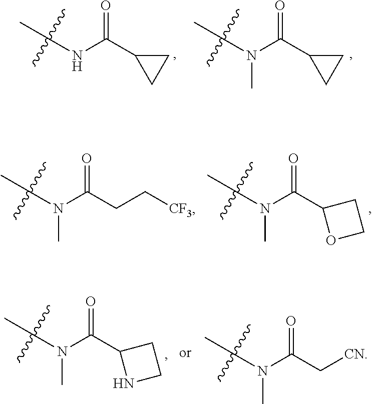

In some embodiments of the compounds of Formula IV and stereoisomers and pharmaceutically acceptable salts thereof, R^{4D5} is H. In some embodiments, R^{4D5} is C₁₋₆alkyl. In some embodiments, R^{4D6} is C₁₋₆alkyl. In some embodiments, R^{4D6} is cycloalkyl. In some embodiments, R^{4D6} is heterocyclyl. In some embodiments, R^{4D5} is H and R^{4D6} is C₁₋₆alkyl, cycloalkyl, or heterocyclyl.

In some embodiments, the compound is of Formula V:

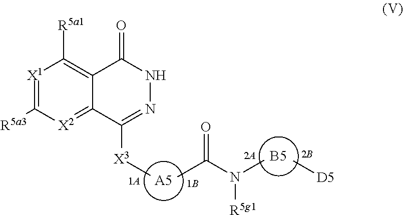

and stereoisomers and pharmaceutically acceptable salts thereof, wherein:
X¹ is —N— or —CR^{5a2}—;
X² is —N— or —CR^{5a4}—;
R^{5a1}, R^{5a2}, R^{5a3}, and R^{5a4} are independently H, halo, —OH, C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, —SO₂-cycloalkyl, —NR^{5L1}R^{5L2}, or C₂₋₆alkynyl-NR^{5L3}R^{5L4};
wherein if X¹ is —CR^{5a2}— and X² is —CR^{5a4}—, then at least one of R^{5a1}, R^{5a2}, R^{5a3}, and R^{5a4} is not H;
wherein each C₁₋₆alkyl, C₂₋₆alkenyl, C₂₋₆alkynyl, cycloalkyl, heterocyclyl, aryl, heteroaryl, —O—C₁₋₆ alkyl, —O—C₂₋₆alkenyl, —O—C₂₋₆alkynyl, —O-cycloalkyl, —O-heterocyclyl, —O-aryl, —O-heteroaryl, or —SO₂-cycloalkyl of R^{5a1}, R^{5a2}, R^{5a3}, and R^{5a4} is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂;
R^{5L1}, R^{5L2}, R^{5L3}, and R^{5L4} are independently H, C₁₋₆alkyl. C₂₋₆alkynyl, or cycloalkyl, wherein each C₁₋₆alkyl, C₂₋₆alkynyl, or cycloalkyl of R^{5L1}, R^{5L2}, R^{5L3}, and R^{5L4} is unsubstituted or substituted with one or more substituents independently selected from the group consisting of halo, C₁₋₆alkyl, C₁₋₆haloalkyl, cycloalkyl, and halocycloalkyl;
X³ is —O—, —NR^{5b1}—, or —CR^{5b2}R^{5b3}—, wherein R^{5b1}, R^{5b2}, and R^{5b3} are independently H or C₁₋₆alkyl;
A5 is

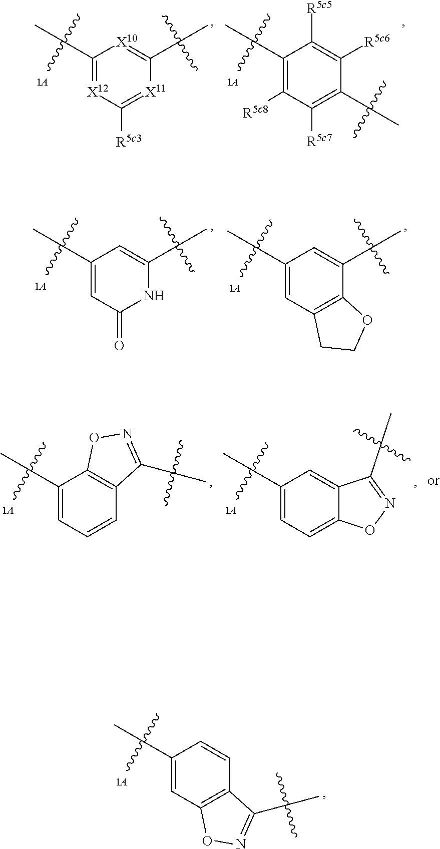

wherein a bond marked 1A is to X³;
X¹⁰ is —N— or —CR^{5c1}—, X¹¹ is —N— or —CR^{5c2}—, and X¹² is —N— or —CR^{5c4}—;
R^{5c1}, R^{5c2}, R^{5c3}, R^{5c4}, R^{5c5}, R^{5c6}, R^{5c7}, and R^{5c8} are independently H, halo, —CN, —SO₂CH₃, —SO₂NH₂, or —NHSO₂CH₃;
R^{5g1} is H or C₁₋₆alkyl;
B5 is a 3 to 8-membered monocyclic heterocyclediyl, a 7 to 18-membered polycyclic heterocyclediyl, or a 7 to 18-membered spirocyclic heterocyclediyl;
wherein the 3 to 8-membered monocyclic heterocyclediyl, 7 to 18-membered polycyclic heterocyclediyl, or 7 to 18-membered spirocyclic heterocyclediyl of B5 is unsubstituted or substituted with one or more substituents selected from the group consisting of halo, C₁₋₆alkyl, and oxo;
D5 is C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, —C(O)-heteroaryl, —N(R^{5D1})(R^{5D2}), —C(O)N(R^{5D3})(R^{5D4}), or —N(R^{5D5})C(O)R^{5D6};
wherein the C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, or —C(O)-heteroaryl of D5 is unsubstituted or substituted with one or more substituents selected from the group consisting of halo, —CN, C₁₋₆alkyl, C₁₋₆ haloalkyl, cycloalkyl, —OH, —O—C₁₋₆alkyl, C₁₋₆alkyl-OH, —O—C₁₋₆ haloalkyl, C₁₋₆alkyl-O—C₁₋₆alkyl, C₁₋₆alkyl-O—C₁₋₆haloalkyl, —C(O)-cycloalkyl, and —C(O)N(R^{5D10})₂, wherein each R^{5D10} is independently H or C₁₋₆ alkyl;
R^{5D1}, R^{5D3}, and R^{5D5} are independently H or C₁₋₆alkyl;
R^{5D2} is aryl or heteroaryl, wherein the aryl or heteroaryl of R^{5D2} is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{5D11})₂, wherein each R^{5D11} is independently H or C₁₋₆alkyl; and
R^{5D4} and R^{5D6} are independently C₁₋₆alkyl, cycloalkyl, heterocyclyl, aryl, or heteroaryl, wherein each C₁₋₆alkyl, cycloalkyl, heterocyclyl, aryl, or heteroaryl of R^{5D4} and R^{5D6} is unsubstituted or substituted with one or more substituents independently selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆ haloalkyl, —C(O)-cycloalkyl, and —C(O)N(R^{5D12})₂, wherein each R^{5D12} is independently H or C₁₋₆alkyl. In some embodiments of the compounds of Formula V and stereoisomers and pharmaceutically acceptable salts thereof. X¹ is —CR^{5a2}—. In some embodiments, X² is —CR^{5a4}—. In some embodiments, X¹ is —N—. In some embodiments. X² is —N—. In some embodiments, X¹ is —CR^{5a2}— and X² is —CR^{5a4}—. In some embodiments, X¹ is —CR^{5a2}— and X² is —N—. In some embodiments, X¹ is —N— and X² is —CR^{5a4}—.

In some embodiments of the compounds of Formula V and stereoisomers and pharmaceutically acceptable salts thereof, one or more of R^{5a1}, R^{5a2}, R^{5a3}, and R^{5a4} is not H. In some embodiments, R^{5a1} is not H. In some embodiments, R^{5a2} is not H. In some embodiments, R^{5a3} is not H. In some embodiments, R^{5a4} is not H.

In some embodiments of the compounds of Formula V and stereoisomers and pharmaceutically acceptable salts thereof, one or more of R^{5a1}, R^{5a2}, R^{5a3}, and R^{5a4} is halo. In some embodiments, R^{5a1} is halo. In some embodiments, R^{5a2} is halo. In some embodiments, R^{5a3} is halo. In some embodiments, R^{5a4} is halo. In some embodiments, the halo is F, Cl, or Br. In some embodiments, the halo is Cl. In some embodiments, the halo is F. In some embodiments, R^{5a1} is F. In some embodiments, R^{5a3} is F. In some embodiments, R^{5a3} is F.

In some embodiments of the compounds of Formula V and stereoisomers and pharmaceutically acceptable salts thereof, one or more of R^{5a1}, R^{5a2}, R^{5a3}, and R^{5a4} is —OH. In some embodiments, R^{5a1} is —OH. In some embodiments, R^{5a2} is —OH. In some embodiments, R^{5a3} is —OH. In some embodiments, R^{5a4} is —OH.

In some embodiments of the compounds of Formula V and stereoisomers and pharmaceutically acceptable salts thereof, one or more of R^{5a1}, R^{5a2}, R^{5a3}, and R^{5a4} is C₁₋₆alkyl. In some embodiments, R^{5a1} is C₁₋₆alkyl. In some embodiments, R^{5a2} is C₁₋₆alkyl. In some embodiments, R^{5a3} is C₁₋₆alkyl. In some embodiments, R^{5a4} is C₁₋₆alkyl. In some embodiments, the C₁₋₆alkyl is unsubstituted or substituted with one or more substituents independently selected from the group consisting of halo, —OH, C₁₋₆alkyl, C₁₋₆ haloalkyl, cycloalkyl, halocycloalkyl, —O—C₁₋₆alkyl, and —C(O)NH₂. In some embodiments, the C₁₋₆alkyl is substituted with halo. In some embodiments, R^{5a1}, R^{5a2}, R^{5a3}, or R^{5a4} is —CH₃, —CH₂CH₃, —CH₂CF₃, —CF₃, —CF₂CH₃, —CH₂CH₂CF₃,

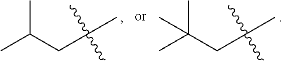

In some embodiments of the compounds of Formula V and stereoisomers and pharmaceutically acceptable salts thereof, one or more of R^{5a1}, R^{5a2}, R^{5a3}, and R^{5a4} is C₂₋₆alkenyl. In some embodiments, R^{5a1} is C₂₋₆alkenyl. In some embodiments, R^{5a2} is C₂₋₆alkenyl. In some embodiments, R^{5a3} is C₂₋₆alkenyl. In some embodiments, R^{5a4} is C₂₋₆-alkenyl. In some embodiments, the C₂₋₆alkenyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the C₂₋₆alkenyl is substituted with halo. In some embodiments, R^{5a1}, R^{5a2}, R^{5a3}, or R^{5a4} is

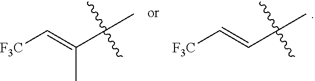

In some embodiments of the compounds of Formula V and stereoisomers and pharmaceutically acceptable salts thereof, one or more of R^{5a1}, R^{5a2}, R^{5a3}, and R^{5a4} is C₂₋₆alkynyl. In some embodiments, R^{5a1} is C₂₋₆alkynyl. In some embodiments, R^{5a2} is C₂₋₆alkynyl. In some embodiments, R^{5a3} is C₁₋₆alkynyl. In some embodiments, R^{5a4} is C₂₋₆alkynyl. In some embodiments, the C₂₋₆alkynyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the C₂₋₆alkynyl is substituted with halo. In some embodiments, the C₂₋₆alkynyl is substituted with cycloalkyl. In some embodiments, R^{5a1}, R^{5a2}, R^{5a3}, or R^{5a4} is

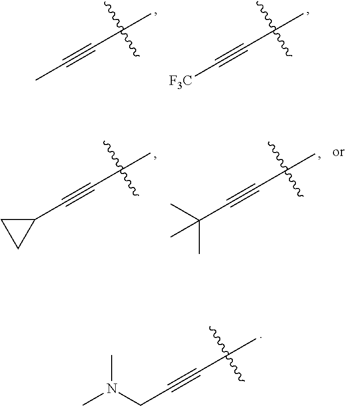

In some embodiments of the compounds of Formula V and stereoisomers and pharmaceutically acceptable salts thereof, one or more of R^{5a1}, R^{5a2}, R^{5a3}, and R^{5a4} is cycloalkyl. In some embodiments, R^{5a1} is cycloalkyl. In some embodiments, R^{5a2} is cycloalkyl. In some embodiments, R^{5a3} is cycloalkyl. In some embodiments, R^{5a4} is cycloalkyl. In some embodiments, the cycloalkyl is unsubstituted or substituted with one or more substituents independently selected from the group consisting of halo, —OH, C₁₋₆alkyl, C₁₋₆ haloalkyl, cycloalkyl, halocycloalkyl, —O—C₁₋₆alkyl, and —C(O)NH₂. In some embodiments, the cycloalkyl is substituted with halo. In some embodiments, R^{5a1}, R^{5a2}, R^{5a3}, or R^{5a4} is

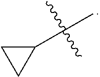 [SMILES: *C1CC1]

In some embodiments of the compounds of Formula V and stereoisomers and pharmaceutically acceptable salts thereof, one or more of R^{5a1}, R^{5a2}, R^{5a3}, and R^{5a4} is heterocyclyl. In some embodiments, R^{5a1} is heterocyclyl. In some embodiments, R^{5a2} is heterocyclyl. In some embodiments, R^{5a3} is heterocyclyl. In some embodiments, R^{5a4} is heterocyclyl. In some embodiments, the heterocyclyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the heterocyclyl is substituted with halo. In some embodiments, the heterocyclyl is substituted with —OH. In some embodiments, R^{5a1}, R^{5a2}, R^{5a3}, or R^{5a4} is

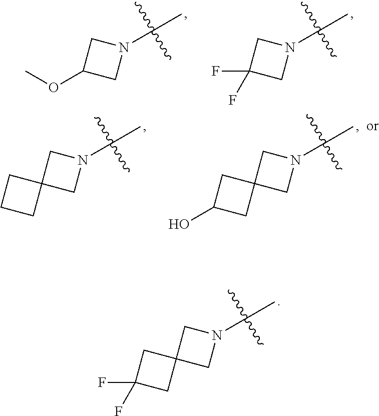

In some embodiments of the compounds of Formula V and stereoisomers and pharmaceutically acceptable salts thereof, one or more of R^{5a1}, R^{5a2}, R^{5a3}, and R^{5a4} is aryl or heteroaryl. In some embodiments, R^{5a1} is aryl or heteroaryl. In some embodiments, R^{5a2} is aryl or heteroaryl. In some embodiments, R^{5a3} is aryl or heteroaryl. In some embodiments, R^{5a4} is aryl or heteroaryl. In some embodiments, the aryl or heteroaryl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, R^{5a1}, R^{5a2}, R^{5a3}, or R^{5a4} is

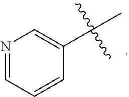 [SMILES: *c1cccnc1]

In some embodiments of the compounds of Formula V and stereoisomers and pharmaceutically acceptable salts thereof, one or more of R^{5a1}, R^{5a2}, R^{5a3}, and R^{5a4} is —O—C₁₋₆alkyl. In some embodiments, R^{5a1} is —O—C₁₋₆alkyl. In some embodiments, R^{5a2} is —O—C₁₋₆alkyl. In some embodiments, R^{5a3} is —O—C₁₋₆alkyl. In some embodiments, R^{5a4} is —O—C₁₋₆alkyl. In some embodiments, the —O—C₁₋₆alkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O—C₁₋₆alkyl is substituted with halo. In some embodiments, the —O—C₁₋₆alkyl is substituted with cycloalkyl. In some embodiments, R^{5a1}, R^{5a2}, R^{5a3}, or R^{5a4} is —OCH₃, —OCHF₂, —OCH₂CF₃, or

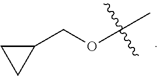 [SMILES: *OCC1CC1]

In some embodiments of the compounds of Formula V and stereoisomers and pharmaceutically acceptable salts thereof, one or more of R^{5a1}, R^{5a2}, R^{5a3}, and R^{5a4} is —O—C₂₋₆alkenyl. In some embodiments, R^{5a1} is —O—C₂₋₆alkenyl. In some embodiments, R^{5a2} is —O—C₂₋₆alkenyl. In some embodiments, R^{5a3} is —O—C₂₋₆ alkenyl. In some embodiments, R^{5a4} is —O—C₂₋₆alkenyl. In some embodiments, the —O—C₂₋₆alkenyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the O—C₂₋₆alkenyl is substituted with halo.

In some embodiments of the compounds of Formula V and stereoisomers and pharmaceutically acceptable salts thereof, one or more of R^{5a1}, R^{5a2}, R^{5a3}, and R^{5a4} is —O—C₂₋₆alkynyl. In some embodiments, R^{5a1} is —O—C₂₋₆alkynyl. In some embodiments, R^{5a2} is —O—C₂₋₆alkynyl. In some embodiments, R^{5a3} is —O—C₂₋₆alkynyl. In some embodiments, R^{5a4} is —O—C₂₋₆alkynyl. In some embodiments, the —O—C₂₋₆alkynyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O—C₂₋₆alkynyl is substituted with halo. In some embodiments, R^{5a1}, R^{5a2}, R^{5a3}, or R^{5a4} is

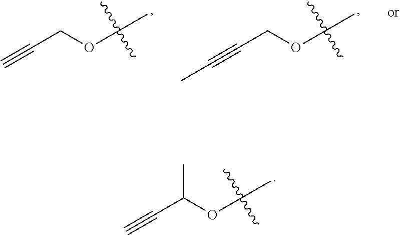

In some embodiments of the compounds of Formula V and stereoisomers and pharmaceutically acceptable salts thereof, one or more of R^{5a1}, R^{5a2}, R^{5a3}, and R^{5a4} is —O-cycloalkyl. In some embodiments, R^{5a1} is —O-cycloalkyl. In some embodiments, R^{5a2} is —O-cycloalkyl. In some embodiments, R^{5a3} is —O-cycloalkyl. In some embodiments, R^{5a4} is cycloalkyl. In some embodiments, the —O-cycloalkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-cycloalkyl is substituted with halo. In some embodiments, the —O-cycloalkyl is substituted with C₁₋₆haloalkyl. In some embodiments, R^{5a1}, R^{5a2}, R^{5a3}, or R^{5a4} is

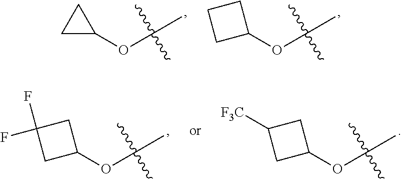 [SMILES: *OC1CC1 ; *OC1CCC1 ; *OC1CC(F)(F)C1 ; *OC1CC(C(F)(F)F)C1]

In some embodiments of the compounds of Formula V and stereoisomers and pharmaceutically acceptable salts thereof, one or more of R^{5a1}, R^{5a2}, R^{5a3}, and R^{5a4} is —O-heterocyclyl. In some embodiments, R^{5a1} is —O-heterocyclyl. In some embodiments, R^{5a2} is —O-heterocyclyl. In some embodiments, R^{5a3} is —O-heterocyclyl. In some embodiments, R^{5a4} is —O-heterocyclyl. In some embodiments, the —O-heterocyclyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-heterocyclyl is substituted with halo.

In some embodiments of the compounds of Formula V and stereoisomers and pharmaceutically acceptable salts thereof, one or more of R^{5a1}, R^{5a2}, R^{5a3}, and R^{5a4} is —O-aryl. In some embodiments, R^{5a1} is —O-aryl. In some embodiments, R^{5a2} is —O-aryl. In some embodiments, R^{5a3} is —O-aryl. In some embodiments, R^{5a4} is —O-aryl. In some embodiments, the —O-aryl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-aryl is substituted with halo. In some embodiments, the —O-aryl is substituted with C₁₋₆alkyl. In some embodiments, the —O-aryl is substituted with —C(O)NH₂. In some embodiments, the aryl of is a 6 membered monocyclic aryl. In some embodiments, R is —O-aryl wherein the aryl is an unsubstituted 6-membered monocyclic aryl. In some embodiments, R^{5a1}, R^{5a2}, R^{5a3}, or R^{5a4} is

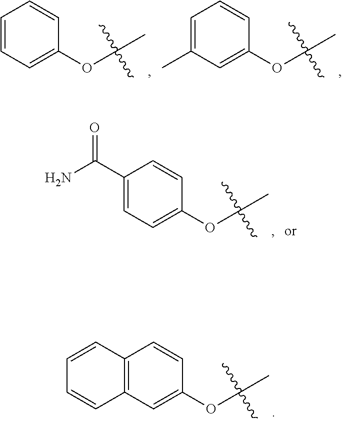

In some embodiments of the compounds of Formula V and stereoisomers and pharmaceutically acceptable salts thereof, one or more of R^{5a1}, R^{5a2}, R^{5a3}, and R^{5a4} is —O-heteroaryl. In some embodiments, R^{5a1} is —O-heteroaryl. In some embodiments, R^{5a2} is —O-heteroaryl. In some embodiments, R^{5a3} is —O-heteroaryl. In some embodiments, R^{5a4} is —O-heteroaryl. In some embodiments, the —O-heteroaryl is unsubstituted or substituted with one or more substituents independently selected from the group consisting of halo, —OH, C₁₋₆ alkyl, C₁₋₆haloalkyl, cycloalkyl, halocycloalkyl, —O—C₁₋₆alkyl, and —C(O)NH₂.

In some embodiments, the —O-heteroaryl is substituted with halo. In some embodiments, R^{5a1}, R^{5a2}, R^{5a3}, or R^{5a4} is

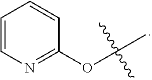 [SMILES: *Oc1ccccn1]

In some embodiments of the compounds of Formula V and stereoisomers and pharmaceutically acceptable salts thereof, one or more of R^{5a1}, R^{5a2}, R^{5a3}, and R^{5a4} is —SO₂-cycloalkyl. In some embodiments, R^{5a1} is —SO₂-cycloalkyl. In some embodiments, R^{5a2} is —SO₂-cycloalkyl. In some embodiments, R^{5a3} is —SO₂-cycloalkyl. In some embodiments, R^{5a4} is —SO₂-cycloalkyl. In some embodiments, the —SO₂-cycloalkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —SO₂-cycloalkyl is substituted with halo. In some embodiments, R^{5a1}, R^{5a2}, R^{5a3}, or R^{5a4} is

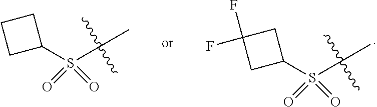 [SMILES: *S(=O)(=O)C1CCC1 ; *S(=O)(=O)C1CC(F)(F)C1]

In some embodiments of the compounds of Formula V and stereoisomers and pharmaceutically acceptable salts thereof, one or more of R^{5a1}, R^{5a2}, R^{5a3}, and R^{5a4} is —NR^{5L1}R^{5L2}. In some embodiments, R^{5a1} is —NR^{5L1}R^{5L2}. In some embodiments, R^{5a2} is —NR^{5L1}R^{5L2}. In some embodiments, R^{5a3} is —NR^{5L1}R^{5L2}. In some embodiments, R^{5a4} is —NR^{5L1}R^{5L2}. In some embodiments, R^{5a1}, R^{5a2}, R^{5a3}, or R^{5a4} is

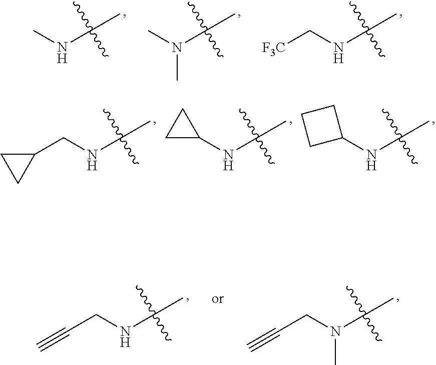

In some embodiments of the compounds of Formula V and stereoisomers and pharmaceutically acceptable salts thereof, R^{5L1} and R^{5L2} are independently H, C₁₋₆alkyl, C₂₋₆alkynyl, or cycloalkyl. In some embodiments, R^{5L1} is H. In some embodiments, R^{5L1} is H and R^{5L2} is C₁₋₆alkyl, C₂₋₆alkynyl, or cycloalkyl. In some embodiments, R^{5L1} is —CH₃. In some embodiments, R^{5L1} is —CH₃ and R^{5L2} is C₁₋₆alkyl, C₂₋₆alkynyl, or cycloalkyl. In some embodiments, R^{5L1} is H or —CH₃ and R^{5L2} is C₁₋₆alkyl. In some embodiments, R^{5L1} is H or —CH₃ and R^{5L2} is C₂₋₆alkynyl. In some embodiments, R^{5L1} is H or —CH₃ and R^{5L2} is cycloalkyl. In some embodiments, the C₁₋₆alkyl, C₂₋₆alkynyl, or cycloalkyl is unsubstituted or substituted with one or more substituents independently selected from the group consisting of halo, C₁₋₆alkyl, C₁₋₆haloalkyl, cycloalkyl, and halocycloalkyl. In some embodiments, the C₁₋₆alkyl is substituted with halo. In some embodiments, the C₁₋₆alkyl is substituted with cycloalkyl.

In some embodiments of the compounds of Formula V and stereoisomers and pharmaceutically acceptable salts thereof, one or more of R^{5a1}, R^{5a2}, R^{5a3}, and R^{5a4} is C₂₋₆alkynyl-NR^{5L3}R^{5L4}. In some embodiments, R^{5a1} is C₂₋₆alkynyl-NR^{5L3}R^{5L4}. In some embodiments, R^{5a2} is C₂₋₆alkynyl-NR^{5L3}R^{5L4}. In some embodiments, R^{5a3} is C₂₋₆-alkynyl-NR^{5L3}R^{5L4}. In some embodiments, R^{5a4} is C₂₋₆alkynyl-NR^{5L3}R^{5L4}.

In some embodiments of the compounds of Formula V and stereoisomers and pharmaceutically acceptable salts thereof, R^{5L3} and R^{5L4} are independently H, C₁₋₆alkyl, C₂₋₆alkynyl, or cycloalkyl. In some embodiments, R^{5L3} is H. In some embodiments, R^{5L3} is H and R^{5L4} is C₁₋₆alkyl, C₂₋₆-alkynyl, or cycloalkyl. In some embodiments, R^{5L3} is —CH₃. In some embodiments, R^{5L3} is —CH₃ and R^{5L4} is C₁₋₆alkyl, C₂₋₆alkynyl, or cycloalkyl. In some embodiments, R^{5L3} is C₁₋₆alkyl and R^{5L4} is C₁₋₆alkyl. In some embodiments, R^{5L3} is H or —CH₃ and R^{5L4} is C₁₋₆alkyl. In some embodiments, R^{5L3} is H or —CH₃ and R^{5L4} is C₂₋₆alkynyl. In some embodiments, R^{5L3} is H or —CH₃ and R^{5L4} is cycloalkyl. In some embodiments, the C₁₋₆alkyl, C₂₋₆alkynyl, or cycloalkyl is unsubstituted or substituted with one or more substituents independently selected from the group consisting of halo, C₁₋₆ alkyl, C₁₋₆haloalkyl, cycloalkyl, and halocycloalkyl. In some embodiments, the C₁₋₆alkyl is substituted with halo. In some embodiments, the C₁₋₆alkyl is substituted with cycloalkyl. In some embodiments, R^{5a1}, R^{5a2}, R^{5a3}, or R^{5a4} is

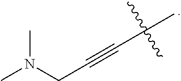 [SMILES: *C#CCN(C)C]

In some embodiments of the compounds of Formula V and stereoisomers and pharmaceutically acceptable salts thereof, two or more of R^{5a1}, R^{5a2}, R^{5a3}, and R^{5a4} is not H. In some embodiments, R^{5a3} is not H and one of R^{5a1}, R^{5a2}, or R^{5a4} is not H. In some embodiments, R^{5a2} is F or Cl and R^{5a3} is —O—C₁₋₆alkyl or —O-cycloalkyl, wherein the —O—C₁₋₆alkyl or —O-cycloalkyl is unsubstituted or substituted with halo or —CN. In some embodiments, R^{5a1} is not H and R^{5a2} is not H. In some embodiments, R^{5a1} is F or Cl and R^{5a2} is F or Cl.

In some embodiments of the compounds of Formula V and stereoisomers and pharmaceutically acceptable salts thereof. X³ is —O—. In some embodiments, X³ is —NR^{5b1}. In some embodiments, X³ is —NH—. In some embodiments, X³ is —N(CH₃)—. In some embodiments, X³ is —CR^{5b2}R^{5b3}—. In some embodiments, X³ is —CH(CH₃)—. In some embodiments, X³ is —CD₂-. In some embodiments, X³ is —CH₂—.

In some embodiments of the compounds of Formula V and stereoisomers and pharmaceutically acceptable salts thereof, A5 is

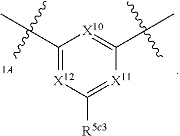

In some embodiments, one or more of X¹⁰, X¹¹, and X¹² is —N—. In some embodiments, X¹⁰ is —N—. In some embodiments, X¹¹ is —N—. In some embodiments, X¹² is —N—. In some embodiments, X¹⁰ is —CR^{5c1}. In some embodiments, X¹¹ is —CR^{5c2}—. In some embodiments, X¹² is —CR^{5c3}—. In some embodiments, X¹⁰ is —CR^{5c1}—, X¹¹ is —CR^{5c2}— and X¹² is —CR^{5c3}.

In some embodiments of the compounds of Formula V and stereoisomers and pharmaceutically acceptable salts thereof, one or more of R^{5c1}, R^{5c2}, R^{5c3}, and R^{5c4} is not H. In some embodiments, one or more of R^{5c1}, R^{5c2}, R^{5c3}, and R^{5c4} is —CN. In some embodiments, one or more of R^{5c1}, R^{5c2}, R^{5c3}, and R^{5c4} is independently Cl, F, or Br. In some embodiments, two or more of R^{5c1}, R^{5c2}, R^{5c3}, and R^{5c4} are independently Cl, F, or Br. In some embodiments, R^{5c2} is F and R^{5c1}, R^{5c3}, and R^{5c4} are H. In some embodiments, R^{5c1}, R^{5c2}, R^{5c3}, and R^{5c4} are H.

In some embodiments of the compounds of Formula V and stereoisomers and pharmaceutically acceptable salts thereof, A5 is

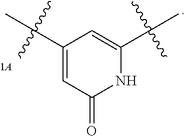 [SMILES: "." ; *c1cc(*)[nH]c(=O)c1]

In some embodiments, A5 is

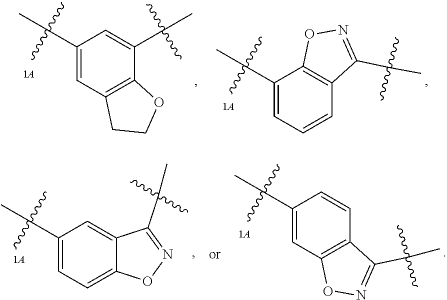 [SMILES: *c1cc(*)c2c(c1)CCO2 ; *c1noc2c(*)cccc12 ; *c1ccc2onc(*)c2c1 ; *c1ccc2c(*)noc2c1]

In some embodiments, A5 is

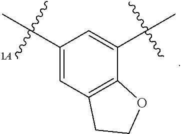 [SMILES: *c1cc(*)c2c(c1)CCO2]

In some embodiments, A5 is

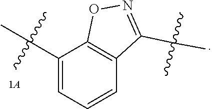 [SMILES: *c1noc2c(*)cccc12]

In some embodiments, A5 is

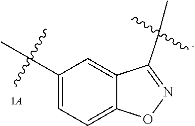 [SMILES: *c1ccc2onc(*)c2c1]

In some embodiments, A5 is

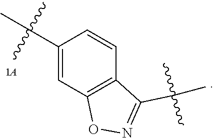 [SMILES: *c1ccc2c(*)noc2c1]

In some embodiments of the compounds of Formula V and stereoisomers and pharmaceutically acceptable salts thereof, B5 is a 3-membered monocyclic heterocyclediyl, a 4-membered monocyclic heterocyclediyl, a 5-membered monocyclic heterocyclediyl comprising 2 or more N, a 6-membered monocyclic heterocyclediyl comprising 2 or more N, a 7-membered monocyclic heterocyclediyl, an 8-membered monocyclic heterocyclediyl, a 7 to 18-membered poly-cyclic heterocyclediyl, or a 7 to 18-membered spirocyclic heterocyclediyl, wherein the 3-membered monocyclic heterocyclediyl, 4-membered monocyclic heterocyclediyl, 5-membered monocyclic heterocyclediyl, 6-membered monocyclic heterocyclediyl, 7-membered monocyclic heterocyclediyl, 8-membered monocyclic heterocyclediyl, 7 to 18-membered polycyclic heterocyclediyl, or 7 to 18-membered spirocyclic heterocyclediyl of B5 is unsubstituted or substituted with one or more substituents independently selected from the group consisting of halo, C₁₋₆alkyl, and oxo. In some embodiments, the 7 to 18-membered polycyclic heterocyclediyl is a 7, 8, 9, 10, 11, 12, 13, 14, 15, 16, 17, or 18-membered polycyclic heterocyclediyl. In some embodiments, the 7 to 18-membered spirocyclic heterocyclediyl is a 7, 8, 9, 10, 11, 12, 13, 14, 15, 16, 17, or 18-membered spirocyclic heterocyclediyl.

In some embodiments of the compounds of Formula V and stereoisomers and pharmaceutically acceptable salts thereof, B5 is a 3 to 8-membered monocyclic heterocyclediyl, wherein the 3 to 8-membered monocyclic heterocyclediyl is unsubstituted or substituted with one or more substituents selected from the group consisting of halo, C₁₋₆alkyl, and oxo. In some embodiments, the 3 to 8-membered monocyclic heterocyclediyl is a 3, 4, 5, 6, 7, or 8-membered monocyclic heterocyclediyl. In some embodiments, B5 is a 4-membered monocyclic heterocyclediyl, wherein the 4-membered monocyclic heterocyclediyl is unsubstituted or substituted with one or more substituents selected from the group consisting of halo, C₁₋₆alkyl, and oxo. In some embodiments, B5 is

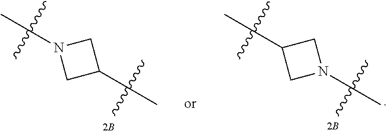 [SMILES: *C1CN(*)C1 ; *C1CN(*)C1]

In some embodiments, B5 is a 6-membered monocyclic heterocyclediyl, wherein the 6-membered monocyclic heterocyclediyl is unsubstituted or substituted with one or more substituents selected from the group consisting of halo, C₁₋₆alkyl, and oxo. In some embodiments, B5 is

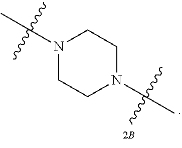 [SMILES: *N1CCN(*)CC1]

In some embodiments, B5 is a 7-membered monocyclic heterocyclediyl, wherein the 7-membered monocyclic heterocyclediyl is unsubstituted or substituted with one or more substituents selected from the group consisting of halo, C₁₋₆alkyl, and oxo. In some embodiments, B5 is

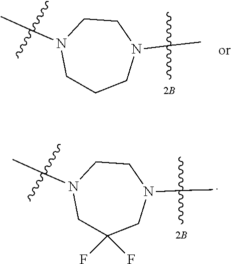 [SMILES: *N1CCCN(*)CC1 ; *N1CCN(*)CC(F)(F)C1]

In some embodiments of the compounds of Formula V and stereoisomers and pharmaceutically acceptable salts thereof, B5 is a 7 to 18-membered polycyclic heterocyclediyl, wherein the 7 to 18-membered polycyclic heterocyclediyl is unsubstituted or substituted with one or more substituents selected from the group consisting of halo, C₁₋₆alkyl, and oxo. In some embodiments, the 7 to 18-membered polycyclic heterocyclediyl is a 7, 8, 9, 10, 11, 12, 13, 14, 15, 16, 17, or 18-membered polycyclic heterocyclediyl. In some embodiments, B5 is

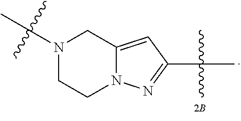 [SMILES: *c1cc2n(n1)CCN(*)C2]

In some embodiments, B5 is

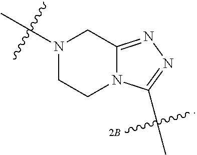 [SMILES: *c1nnc2n1CCN(*)C2]

In some embodiments B5 is

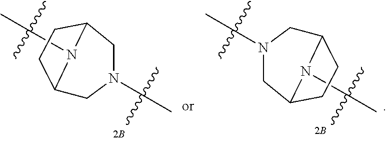 [SMILES: *N1CC2CCC(C1)N2* ; *N1CC2CCC(C1)N2*]

In some embodiments, B5 is

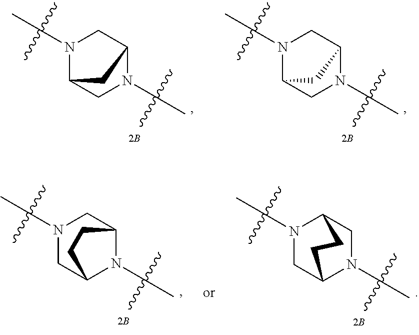

In some embodiments, B5 is

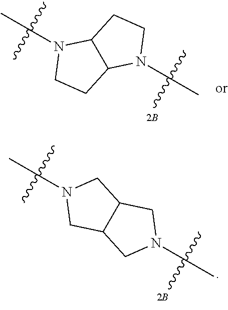 [SMILES: *N1CCC2C1CCN2* ; *N1CC2CN(*)CC2C1]

In some embodiments, B5 is

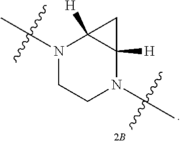 [SMILES: *N1CCN(*)[C@H]2C[C@H]21]

In some embodiments of the compounds of Formula V and stereoisomers and pharmaceutically acceptable salts thereof, B5 is a 7 to 18-membered spirocyclic heterocyclediyl, wherein the 7 to 18-membered spirocyclic heterocyclediyl is unsubstituted or substituted with one or more substituents selected from the group consisting of halo, C₁₋₆alkyl, and oxo. In some embodiments, the 7 to 18-membered spirocyclic heterocyclediyl is a 7, 8, 9, 10, 11, 12, 13, 14, 15, 16, 17, or 18-membered spirocyclic heterocyclediyl. In some embodiments, B5 is

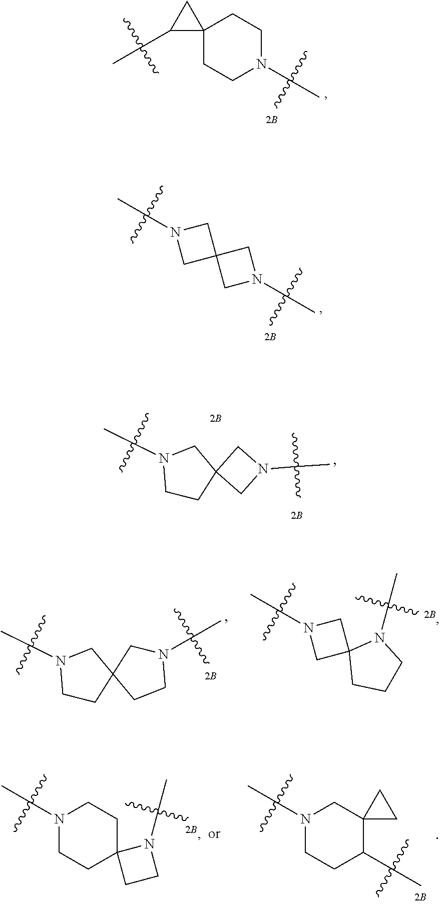

In some embodiments of the compounds of Formula V and stereoisomers and pharmaceutically acceptable salts thereof, the monocyclic heterocyclediyl, polycyclic heterocyclediyl, or spirocyclic heterocyclediyl of B5 comprises one or more N. In some embodiments, the monocyclic heterocyclediyl, polycyclic heterocyclediyl, or spirocyclic heterocyclediyl of B5 comprises two or more N.

In some embodiments of the compounds of Formula V and stereoisomers and pharmaceutically acceptable salts thereof, R^{5g1} is H. In some embodiments, R^{5g1} is C₁₋₆alkyl. In some embodiments, R^{5g1} is —CH₃.

In some embodiments of the compounds of Formula V and stereoisomers and pharmaceutically acceptable salts thereof, D5 is C₁₋₆alkyl, wherein the C₁₋₆alkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{5D10})₂. In some embodiments, the C₁₋₆alkyl is unsubstituted or substituted with halo. In some embodiments, the C₁₋₆alkyl is unsubstituted or substituted with —OH. In some embodiments, the C₁₋₆alkyl is unsubstituted or substituted with —O—C₁₋₆alkyl.

In some embodiments of the compounds of Formula V and stereoisomers and pharmaceutically acceptable salts thereof. D5 is cycloalkyl, wherein the cycloalkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{5D10})₂. In some embodiments, the cycloalkyl is unsubstituted or substituted with halo. In some embodiments, D5 is

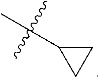 [SMILES: *C1CC1]

In some embodiments of the compounds of Formula V and stereoisomers and pharmaceutically acceptable salts thereof, D5 is aryl, wherein the aryl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{5D10})₂. In some embodiments, the aryl is a monocyclic 6-membered aryl. In some embodiments, the aryl is substituted with one or more substituents selected from the group consisting of halo, —CN, C₁₋₆alkyl, and C₁₋₆haloalkyl. In some embodiments, the aryl is substituted with —CN.

In some embodiments of the compounds of Formula V and stereoisomers and pharmaceutically acceptable salts thereof, D5 is heteroaryl, wherein the heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, —C(O)NH₂, —C(O)NHCH₃, and —C(O)N(CH₃)₂.

In some embodiments of the compounds of Formula V and stereoisomers and pharmaceutically acceptable salts thereof, D5 is a monocyclic 5 or 6-membered heteroaryl comprising one or more N, wherein the monocyclic 5 or 6-membered heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, —C(O)NH₂, —C(O)NHCH₃, and —C(O)N(CH₃)₂. In some embodiments, the monocyclic 5 or 6-membered heteroaryl is substituted with —CN. In some embodiments, the monocyclic 5 or 6-membered heteroaryl is substituted with —CF₃. In some embodiments, the monocyclic 5 or 6-membered heteroaryl is substituted with —OCH₃.

In some embodiments of the compounds of Formula V and stereoisomers and pharmaceutically acceptable salts thereof, D5 is —O—C₁₋₆alkyl, wherein the —O—C₁₋₆alkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆ haloalkyl, —C(O)-cycloalkyl, and —C(O)N(R^{5D10})₂. In some embodiments, the —O—C₁₋₆alkyl is unsubstituted or substituted with halo.

In some embodiments of the compounds of Formula V and stereoisomers and pharmaceutically acceptable salts thereof, D5 is —O-aryl or —O-heteroaryl, wherein the —O-aryl or —O-heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆ alkyl, C₁₋₆alkyl-O—C₁₋₆haloalkyl, —C(O)-cycloalkyl, and —C(O)N(R^{5D10})₂. In some embodiments, the —O-aryl or —O-heteroaryl is unsubstituted or substituted with halo. In some embodiments, the —O-aryl or —O-heteroaryl is unsubstituted or substituted with —CN. In some embodiments, D5 is

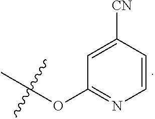 [SMILES: *Oc1cc(C#N)ccn1]

In some embodiments of the compounds of Formula V and stereoisomers and pharmaceutically acceptable salts thereof, D5 is —C(O)—C₁₋₆alkyl, wherein the —C(O)—C₁₋₆alkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{5D10})₂. In some embodiments, the —C(O)—C₁₋₆alkyl is unsubstituted or substituted with halo. In some embodiments, the —C(O)—C₁₋₆alkyl is unsubstituted or substituted with —CN. In some embodiments, D5 is

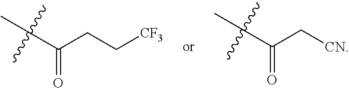 [SMILES: *C(=O)CCC(F)(F)F ; *C(=O)CC#N]

In some embodiments of the compounds of Formula V and stereoisomers and pharmaceutically acceptable salts thereof, D5 is —C(O)-cycloalkyl, wherein the —C(O)-cycloalkyl is unsubstituted or substituted with one or more substituents selected from the group consisting of halo, —CN, C₁₋₆alkyl, C₁₋₆ haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{5D10})₂. In some embodiments, the —C(O)-cycloalkyl is unsubstituted or substituted with halo. In some embodiments, D5 is

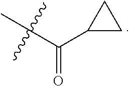 [SMILES: *C(=O)C1CC1]

In some embodiments of the compounds of Formula V and stereoisomers and pharmaceutically acceptable salts thereof, D5 is —C(O)-heterocyclyl, wherein the —C(O)-heterocyclyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{5D10})₂. In some embodiments, the —C(O)-heterocyclyl is unsubstituted or substituted with halo. In some embodiments, D5 is

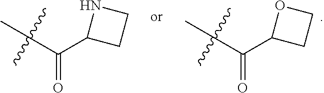 [SMILES: *C(=O)C1CCN1 ; *C(=O)C1CCO1]

In some embodiments of the compounds of Formula V and stereoisomers and pharmaceutically acceptable salts thereof, D5 is —C(O)-aryl or —C(O)-heteroaryl, wherein the —C(O)-aryl or —C(O)-heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{5D10})₂. In some embodiments, the —C(O)-aryl or —C(O)-heteroaryl is unsubstituted or substituted with halo. In some embodiments, the —C(O)-aryl or —C(O)-heteroaryl is unsubstituted or substituted with —CF₃. In some embodiments, the —C(O)-aryl or —C(O)-heteroaryl is unsubstituted or substituted with —CN. In some embodiments, D5 is

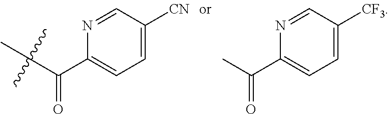

In some embodiments of the compounds of Formula V and stereoisomers and pharmaceutically acceptable salts thereof, D5 is —N(R^{5D1})(R^{5D2}). In some embodiments, D5 is

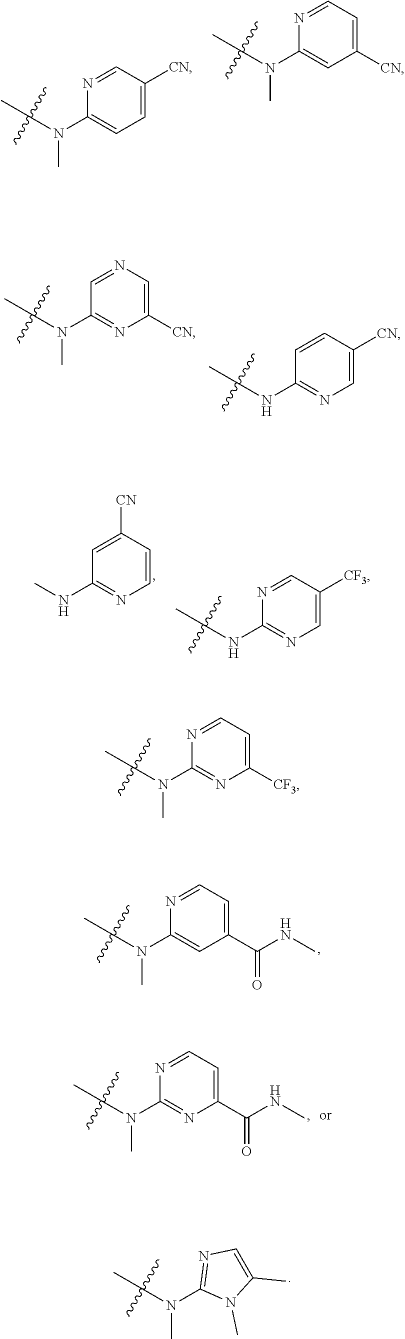

In some embodiments of the compounds of Formula V and stereoisomers and pharmaceutically acceptable salts thereof, R^{5D1} is H. In some embodiments, R^{5D1} is C₁₋₆alkyl. In some embodiments, R^{5D2} is aryl. In some embodiments, R^{5D2} is heteroaryl. In some embodiments, the heteroaryl is a 6-membered heteroaryl.

In some embodiments of the compounds of Formula V and stereoisomers and pharmaceutically acceptable salts thereof, D5 is —C(O)N(R^{5D3})(R^{5D4}). In some embodiments, D5 is

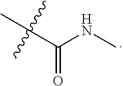 [SMILES: *C(=O)NC]

In some embodiments of the compounds of Formula V and stereoisomers and pharmaceutically acceptable salts thereof, R^{5D3} is H. In some embodiments, R^{5D3} is C₁₋₆alkyl. In some embodiments, R^{5D4} is C₁₋₆alkyl. In some embodiments, R^{5D3} is H and R^{5D4} is C₁₋₆alkyl.

In some embodiments of the compounds of Formula V and stereoisomers and pharmaceutically acceptable salts thereof, D5 is —N(R^{5D5})C(O)R^{5D6}—. In some embodiments, D5 is

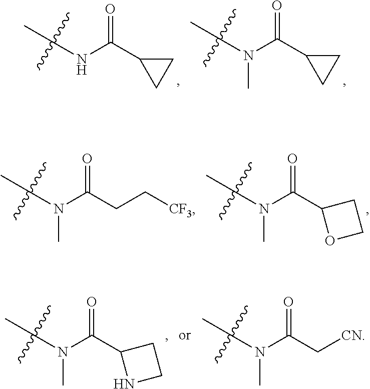

In some embodiments of the compounds of Formula V and stereoisomers and pharmaceutically acceptable salts thereof, R^{5D5} is H. In some embodiments, R^{5D5} is C₁₋₆alkyl. In some embodiments, R^{5D6} is C₁₋₆alkyl. In some embodiments, R^{5D6} is cycloalkyl. In some embodiments, R^{5D6} is heterocyclyl. In some embodiments, R^{5D5} is H and R^{5D6} is C₁₋₆alkyl, cycloalkyl, or heterocyclyl.

In some embodiments, the compound is of Formula VI:

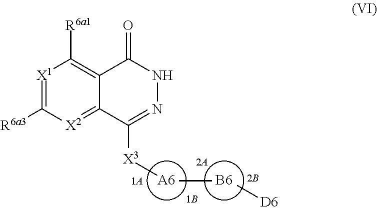

and stereoisomers and pharmaceutically acceptable salts thereof, wherein:
X¹ is —N— or —CR^{6a2}—;
X² is —N— or —CR^{6a4}—;
R^{6a1}, R^{6a2}, R^{6a3}, and R^{6a4} are independently H, halo, —OH, C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, —SO₂-cycloalkyl, —NR^{6L1}R^{6L2}, or C₂₋₆alkynyl-NR^{6L3}R^{6L4};
wherein if X¹ is —CR^{6a2}— and X² is —CR^{6a4}—, then at least one of R^{6a1}, R^{6a2}, R^{6a3}, and R^{6a4} is not H;
wherein each C₁₋₆alkyl, C₂₋₆alkenyl, C₂₋₆alkynyl, cycloalkyl, heterocyclyl, aryl, heteroaryl, —O—C₁₋₆ alkyl, —O—C₂₋₆alkenyl, —O—C₂₋₆alkynyl, —O-cycloalkyl, —O-heterocyclyl, —O-aryl, —O-heteroaryl, or —SO₂-cycloalkyl of R^{6a1}, R^{6a2}, R^{6a3}, and R^{6a4} is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂;
R^{6L1}, R^{6L2}, R^{6L3}, and R^{6L4} are independently H, C₁₋₆alkyl, C₂₋₆alkynyl, or cycloalkyl, wherein each C₁₋₆alkyl, C₂₋₆alkynyl, or cycloalkyl of R^{6L1}, R^{6L2}, R^{6L3}, and R^{6L4} is unsubstituted or substituted with one or more substituents independently selected from the group consisting of halo, C₁₋₆alkyl, C₁₋₆ haloalkyl, cycloalkyl, and halocycloalkyl;
X³ is —O—, —NR^{6b1}—, or —CR^{6b2}R^{6b3}—, wherein R^{6b1}, R^{6b2}, and R^{6b3} are independently H or C₁₋₆alkyl;
A6 is

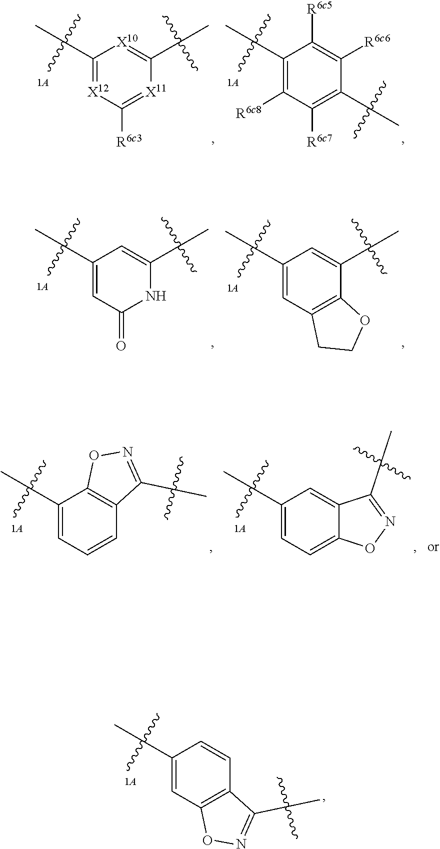

wherein a bond marked 1A is to X³;
X¹⁰ is —N— or —CR^{6c1}—, X¹¹ is —N— or —CR^{6c2}—, and X¹² is —N— or —CR^{6c4}—;
R^{6c1}, R^{6c2}, R^{6c3}, R^{6c4}, R^{6c5}, R^{6c6}, R^{6c7}, and R^{6c8} are independently H, halo, —CN, —SO₂CHA, —SO₂NH₂, or —NHSO₂CH₃;
B6 is a 3 to 8-membered monocyclic heterocyclediyl, a 7 to 18-membered polycyclic heterocyclediyl, or a 7 to 18-membered spirocyclic heterocyclediyl;
wherein the 3 to 8-membered monocyclic heterocyclediyl, 7 to 18-membered polycyclic heterocyclediyl, or 7 to 18-membered spirocyclic heterocyclediyl of B6 is unsubstituted or substituted with one or more substituents selected from the group consisting of halo, C₁₋₆alkyl, and oxo;
D6 is C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, —C(O)-heteroaryl, —N(R^{6D1})(R^{6D2}), —C(O)N(R^{6D3})(R^{6D4}), or —N(R^{6D5})C(O)R^{6D6};
wherein the C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, or —C(O)-heteroaryl of D6 is unsubstituted or substituted with one or more substituents selected from the group consisting of halo, —CN, C₁₋₆alkyl, C₁₋₆haloalkyl, cycloalkyl, —OH, —O—C alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{6D10})₂, wherein each R^{6D10} is independently H or C₁₋₆alkyl;
R^{6D1}, R^{6D3}, and R^{6D5} are independently H or C₁₋₆alkyl;
R^{6D2} is aryl or heteroaryl, wherein the aryl or heteroaryl of R^{6D2} is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{6D11})₂, wherein each R^{6D11} is independently H or C₁₋₆alkyl; and
R^{6D4} and R^{6D5} are independently C₁₋₆alkyl, cycloalkyl, heterocyclyl, aryl, or heteroaryl, wherein each C₁₋₆alkyl, cycloalkyl, heterocyclyl, aryl, or heteroaryl of R^{6D4} and R^{6D6} is unsubstituted or substituted with one or more substituents independently selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{6D12})₂, wherein each R^{6D12} is independently H or C₁₋₆alkyl.

In some embodiments of the compounds of Formula VI and stereoisomers and pharmaceutically acceptable salts thereof, X¹ is —CR^{6a2}—. In some embodiments, X² is —CR^{6a4}—. In some embodiments, X¹ is —N—. In some embodiments, X² is —N—. In some embodiments, X¹ is —CR^{6a2}— and X² is —CR^{6a4}—. In some embodiments. X¹ is —CR^{6a2}— and X² is —N—. In some embodiments, X¹ is —N— and X² is —CR^{6a4}—.

In some embodiments of the compounds of Formula VI and stereoisomers and pharmaceutically acceptable salts thereof, one or more of R^{6a1}, R^{6a2}, R^{6a3}, and R^{6a4} is not H. In some embodiments, R^{6a1} is not H. In some embodiments, R^{6a2} is not H. In some embodiments, R^{6a3} is not H. In some embodiments, R^{6a4} is not H.

In some embodiments of the compounds of Formula VI and stereoisomers and pharmaceutically acceptable salts thereof, one or more of R^{6a1}, R^{6a2}, R^{6a3}, and R^{6a4} is halo. In some embodiments, R^{6a1} is halo. In some embodiments, R^{6a2} is halo. In some embodiments, R^{6a3} is halo. In some embodiments, R^{6a4} is halo. In some embodiments, the halo is F, Cl, or Br. In some embodiments, the halo is Cl. In some embodiments, the halo is F. In some embodiments, R^{6a1} is F. In some embodiments, R^{6a3} is F. In some embodiments, R^{6a3} is F.

In some embodiments of the compounds of Formula VI and stereoisomers and pharmaceutically acceptable salts thereof, one or more of R^{6a1}, R^{6a2}, R^{6a3}, and R^{6a4} is —OH. In some embodiments, R^{6a1} is —OH. In some embodiments, R^{6a2} is —OH. In some embodiments, R^{6a3} is —OH. In some embodiments, R^{6a4} is —OH.

In some embodiments of the compounds of Formula VI and stereoisomers and pharmaceutically acceptable salts thereof, one or more of R^{6a1}, R^{6a2}, R^{6a3}, and R^{6a4} is C₁₋₆alkyl. In some embodiments, R^{6a1} is C₁₋₆alkyl. In some embodiments, R^{6a2} is C₁₋₆alkyl. In some embodiments, R^{6a3} is C₁₋₆alkyl. In some embodiments. R^{6a4} is C₁₋₆alkyl. In some embodiments, the C₁₋₆alkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the C₁₋₆alkyl is substituted with halo. In some embodiments, R^{6a1}, R^{6a2}, R^{6a3}, or R^{6a4} is —CH₃, —CH₂CH₃, —CH₂CF₃, —CF₃, —CF₂CH₃, —CH₂CH₂CF₃,

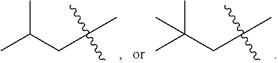

In some embodiments of the compounds of Formula VII and stereoisomers and pharmaceutically acceptable salts thereof, one or more of R^{7a1}, R^{7a2}, R^{7a3}, and R^{7a4} is C₁₋₆alkyl. In some embodiments, R^{7a1} is C₁₋₆alkyl. In some embodiments, R^{7a2} is C₁₋₆alkyl. In some embodiments, R^{7a3} is C₁₋₆alkyl. In some embodiments, R^{7a4} is C₁₋₆alkyl. In some embodiments, the C₁₋₆alkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the C₁₋₆alkyl is substituted with halo. In some embodiments, R^{7a1}, R^{7a2}, R^{7a3}, or R^{7a4} is —CH₃, —CH₂CH₃, —CH₂CF₃, —CF₃, —CF₂CH₃, —CH₂CH₂CF₃,

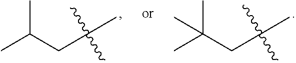

In some embodiments of the compounds of Formula VI and stereoisomers and pharmaceutically acceptable salts thereof, one or more of R^{6a1}, R^{6a2}, R^{6a3}, and R^{6a4} is C₂₋₆alkenyl. In some embodiments, R^{6a1} is C₂₋₆alkenyl. In some embodiments, R^{6a2} is C₂₋₆alkenyl. In some embodiments, R^{6a3} is C₂₋₆alkenyl. In some embodiments, R^{6a4} is C₂₋₆alkenyl. In some embodiments, the C₂₋₆alkenyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the C₂₋₆alkenyl is substituted with halo. In some embodiments, R^{6a1}, R^{6a2}, R^{6a3}, or R^{6a4} is

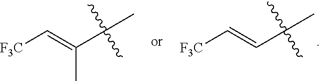

In some embodiments of the compounds of Formula VI and stereoisomers and pharmaceutically acceptable salts thereof, one or more of R^{6a1}, R^{6a2}, R^{6a3}, and R^{6a4} is C₂₋₆alkynyl. In some embodiments, R^{6a1} is C₂₋₆alkynyl. In some embodiments, R^{6a2} is C₂₋₆alkynyl. In some embodiments, R^{6a3} is C₂₋₆alkynyl. In some embodiments, R^{6a4} is C₂₋₆alkynyl. In some embodiments, the C₂₋₆alkynyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the C₂₋₆alkynyl is substituted with halo. In some embodiments, the C₂₋₆alkynyl is substituted with cycloalkyl. In some embodiments, R^{6a1}, R^{6a2}, R^{6a3}, or R^{6a4} is

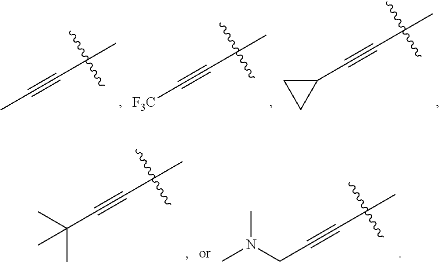

In some embodiments of the compounds of Formula VI and stereoisomers and pharmaceutically acceptable salts thereof, one or more of R^{6a1}, R^{6a2}, R^{6a3}, and R^{6a4} is cycloalkyl. In some embodiments, R^{6a1} is cycloalkyl. In some embodiments, R^{6a2} is cycloalkyl. In some embodiments, R^{6a3} is cycloalkyl. In some embodiments, R^{6a4} is cycloalkyl. In some embodiments, the cycloalkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the cycloalkyl is substituted with halo. In some embodiments, R^{6a1}, R^{6a2}, R^{6a3}, or R^{6a4} is

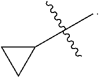 [SMILES: *C1CC1]

In some embodiments of the compounds of Formula VI and stereoisomers and pharmaceutically acceptable salts thereof, one or more of R^{6a1}, R^{6a2}, R^{6a3}, and R^{6a4} is heterocyclyl. In some embodiments, R^{6a1} is heterocyclyl. In some embodiments, R^{6a2} is heterocyclyl. In some embodiments, R^{6a3} is heterocyclyl. In some embodiments, R^{6a4} is heterocyclyl. In some embodiments, the heterocyclyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the heterocyclyl is substituted with halo. In some embodiments, the heterocyclyl is substituted with —OH. In some embodiments, R^{6a1}, R^{6a2}, R^{6a3}, or R^{6a4} is

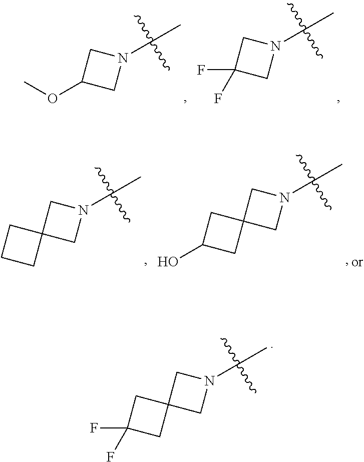

In some embodiments of the compounds of Formula VI and stereoisomers and pharmaceutically acceptable salts thereof, one or more of R^{6a1}, R^{6a2}, R^{6a3}, and R^{6a4} is aryl or heteroaryl. In some embodiments, R^{6a1} is aryl or heteroaryl. In some embodiments, R^{6a2} is aryl or heteroaryl. In some embodiments, R^{6a3} is aryl or heteroaryl. In some embodiments, R^{6a4} is aryl or heteroaryl. In some embodiments, the aryl or heteroaryl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, R^{6a1}, R^{6a2}, R^{6a3}, or R^{6a4} is

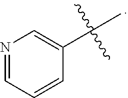 [SMILES: *c1cccnc1]

In some embodiments of the compounds of Formula VI and stereoisomers and pharmaceutically acceptable salts thereof, one or more of R^{6a1}, R^{6a2}, R^{6a3}, and R^{6a4} is —O—C₁₋₆alkyl. In some embodiments, R^{6a1} is —O—C₁₋₆alkyl. In some embodiments, R^{6a2} is —O—C₁₋₆alkyl. In some embodiments, R^{6a3} is —O—C₁₋₆alkyl. In some embodiments, R^{6a4} is —O—C₁₋₆alkyl. In some embodiments, the —O—C₁₋₆alkyl is unsubstituted or substituted with one or more substituents independently selected from the group consisting of halo, —OH, C₁₋₆ alkyl, C₁₋₆haloalkyl, cycloalkyl, halocycloalkyl, —O—C₁₋₆alkyl, and —C(O)NH₂. In some embodiments, the —O—C₁₋₆alkyl is substituted with halo. In some embodiments, the —O—C₁₋₆alkyl is substituted with cycloalkyl. In some embodiments, R^{6a1}, R^{6a2}, R^{6a3}, or R^{6a4} is —OCH₃, —OCHF₂, —OCH₂CF₃, or

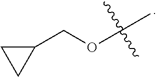 [SMILES: *OCC1CC1]

In some embodiments of the compounds of Formula VI and stereoisomers and pharmaceutically acceptable salts thereof, one or more of R^{6a1}, R^{6a2}, R^{6a3}, and R^{6a4} is —O—C₂₋₆alkenyl. In some embodiments, R^{6a1} is —O—C₂₋₆alkenyl. In some embodiments, R^{6a2} is —O—C₂₋₆alkenyl. In some embodiments, R^{6a3} is —O—C₂₋₆alkenyl. In some embodiments, R^{6a4} is —O—C₂₋₆alkenyl. In some embodiments, the —O—C₁₋₆alkenyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O—C₂₋₆alkenyl is substituted with halo.

In some embodiments of the compounds of Formula VI and stereoisomers and pharmaceutically acceptable salts thereof, one or more of R^{6a1}, R^{6a2}, R^{6a3}, and R^{6a4} is —O—C₂₋₆alkynyl. In some embodiments, R^{6a1} is —O—C₂₋₆alkynyl. In some embodiments, R^{6a2} is —O—C₂₋₆alkynyl. In some embodiments, R^{6a3} is —O—C₂₋₆alkynyl. In some embodiments, R^{6a4} is —O—C₂₋₆alkynyl. In some embodiments, the —O—C₂₋₆alkynyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O—C₂₋₆alkynyl is substituted with halo. In some embodiments, R^{6a1}, R^{6a2}, R^{6a3}, or R^{6a4} is

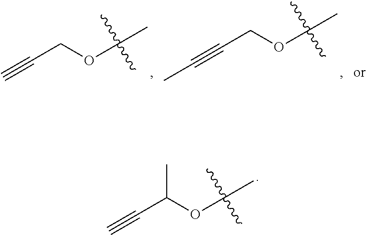

In some embodiments of the compounds of Formula VI and stereoisomers and pharmaceutically acceptable salts thereof, one or more of R^{6a1}, R^{6a2}, R^{6a3}, and R^{6a4} is —O-cycloalkyl. In some embodiments, R^{6a1} is —O-cycloalkyl. In some embodiments, R^{6a2} is —O-cycloalkyl. In some embodiments, R^{6a3} is —O-cycloalkyl. In some embodiments, R^{6a4} is cycloalkyl. In some embodiments, the —O-cycloalkyl is unsubstituted or substituted with one or more substituents independently selected from the group consisting of halo, —OH, C₁₋₆alkyl, C₁₋₆ haloalkyl, cycloalkyl, halocycloalkyl, —O—C₁₋₆alkyl, and —C(O)NH₂. In some embodiments, the —O-cycloalkyl is substituted with halo. In some embodiments, the —O-cycloalkyl is substituted with C₁₋₆haloalkyl. In some embodiments, R^{6a1}, R^{6a2}, R^{6a3}, or R^{6a4} is

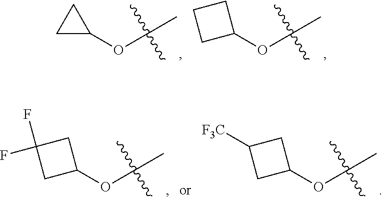 [SMILES: *OC1CC1 ; *OC1CCC1 ; *OC1CC(F)(F)C1 ; *OC1CC(C(F)(F)F)C1]

In some embodiments of the compounds of Formula VI and stereoisomers and pharmaceutically acceptable salts thereof, one or more of R^{6a1}, R^{6a2}, R^{6a3}, and R^{6a4} is —O-heterocyclyl. In some embodiments, R^{6a1} is —O-heterocyclyl. In some embodiments, R^{6a2} is —O-heterocyclyl. In some embodiments, R^{6a3} is —O-heterocyclyl. In some embodiments, R^{6a4} is —O-heterocyclyl. In some embodiments, the —O-heterocyclyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-heterocyclyl is substituted with halo.

In some embodiments of the compounds of Formula VI and stereoisomers and pharmaceutically acceptable salts thereof, one or more of R^{6a1}, R^{6a2}, R^{6a3}, and R^{6a4} is —O-aryl. In some embodiments, R^{6a1} is —O-aryl. In some embodiments, R^{6a2} is —O-aryl. In some embodiments, R^{6a3} is —O-aryl. In some embodiments, R^{6a4} is —O-aryl. In some embodiments, the —O-aryl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-aryl is substituted with halo. In some embodiments, the —O-aryl is substituted with C₁₋₆alkyl. In some embodiments, the —O-aryl is substituted with —C(O)NH₂. In some embodiments, the aryl of is a 6 membered monocyclic aryl. In some embodiments, R^{6a3} is —O-aryl wherein the aryl is an unsubstituted 6-membered monocyclic aryl. In some embodiments, R^{6a1}, R^{6a2}, R^{6a3}, or R^{6a4} is

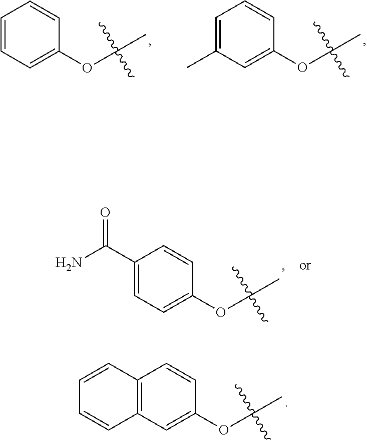

In some embodiments of the compounds of Formula VI and stereoisomers and pharmaceutically acceptable salts thereof, one or more of R^{6a1}, R^{6a2}, R^{6a3}, and R^{6a4} is —O-heteroaryl. In some embodiments, R^{6a1} is —O-heteroaryl. In some embodiments, R^{6a2} is —O-heteroaryl. In some embodiments, R^{6a3} is —O-heteroaryl. In some embodiments, R^{6a4} is —O-heteroaryl. In some embodiments, the —O-heteroaryl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-heteroaryl is substituted with halo. In some embodiments, R^{6a1}, R^{6a2}, R^{6a3}, or R^{6a4} is

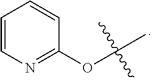 [SMILES: *Oc1ccccn1]

In some embodiments of the compounds of Formula VI and stereoisomers and pharmaceutically acceptable salts thereof, one or more of R^{6a1}, R^{6a2}, R^{6a3}, and R^{6a4} is —SO₂-cycloalkyl. In some embodiments, R^{6a1} is —SO₂-cycloalkyl. In some embodiments, R^{6a2} is —SO₂-cycloalkyl. In some embodiments, R^{6a3} is —SO₂-cycloalkyl. In some embodiments, R^{6a4} is —SO₂-cycloalkyl. In some embodiments, the —SO₂-cycloalkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —SO₂-cycloalkyl is substituted with halo. In some embodiments, R^{6a1}, R^{6a1}, R^{6a3}, or R^{6a4} is

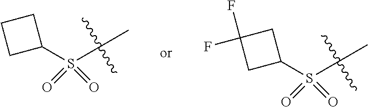 [SMILES: *S(=O)(=O)C1CCC1 ; *S(=O)(=O)C1CC(F)(F)C1]

In some embodiments of the compounds of Formula VI and stereoisomers and pharmaceutically acceptable salts thereof, one or more of R^{6a1}, R^{6a2}, R^{6a3}, and R^{6a4} is —NR^{6L1}R^{6L2}. In some embodiments, R^{6a1} is —NR^{6L1}R^{6L2}. In some embodiments, R^{6a2} is —NR^{6L1}R^{6L2}. In some embodiments, R^{6a3} is —NR^{6L1}R^{6L2}. In some embodiments, R^{6a4} is —NR^{6L1}R^{6L2}. In some embodiments, R^{6a1}, R^{6a2}, R^{6a3}, or R^{6a4} is

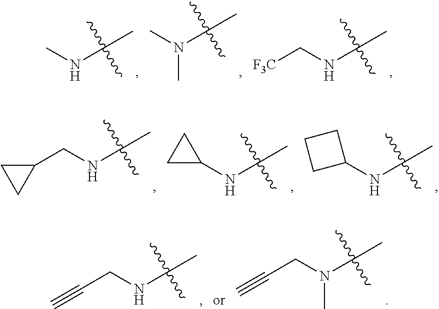

In some embodiments of the compounds of Formula VI and stereoisomers and pharmaceutically acceptable salts thereof, R^{6L1} and R^{6L2} are independently H, C₁₋₆alkyl, C₂₋₆alkynyl, or cycloalkyl. In some embodiments, R^{6L1} is H. In some embodiments, R^{6L1} is H and R^{6L2} is C₁₋₆alkyl, C₂₋₆alkynyl, or cycloalkyl. In some embodiments, R^{6L1} is —CH₃. In some embodiments, R^{6L1} is —CH₃ and R^{6L2} is C₁₋₆alkyl, C₂₋₆alkynyl, or cycloalkyl. In some embodiments, R^{6L1} is H or —CH₃ and R^{6L2} is C₁₋₆alkyl. In some embodiments, R^{6L1} is H or —CH₃ and R^{6L2} is C₂₋₆alkynyl. In some embodiments, R^{6L1} is H or —CH₃ and R^{6L2} is cycloalkyl. In some embodiments, the C₁₋₆alkyl, C₂₋₆alkynyl, or cycloalkyl is unsubstituted or substituted with one or more substituents independently selected from the group consisting of halo, C₁₋₆alkyl, C₁₋₆haloalkyl, cycloalkyl, and halocycloalkyl. In some embodiments, the C₁₋₆alkyl is substituted with halo. In some embodiments, the C₁₋₆alkyl is substituted with cycloalkyl.

In some embodiments of the compounds of Formula VI and stereoisomers and pharmaceutically acceptable salts thereof, one or more of R^{6a1}, R^{6a2}, R^{6a3}, and R^{6a4} is C₂₋₆alkynyl-NR^{6L3}R^{6L4}. In some embodiments, R^{6a1} is C₂₋₆alkynyl-NR^{6L3}R^{6L4}. In some embodiments, R^{6a2} is C₂₋₆alkynyl-NR^{6L3}R^{6L4}. In some embodiments, R^{6a3} is C₂₋₆alkynyl-NR^{6L3}R^{6L4}. In some embodiments, R^{6a4} is C₂₋₆alkynyl-NR^{6L3}R^{6L4}.

In some embodiments of the compounds of Formula VI and stereoisomers and pharmaceutically acceptable salts thereof, R^{6L3} and R^{6L4} are independently H, C₁₋₆alkyl, C₂₋₆-alkynyl, or cycloalkyl. In some embodiments, R^{6L3} is H. In some embodiments, R^{6L3} is H and R^{6L4} is C₁₋₆alkyl, C₂₋₆alkynyl, or cycloalkyl. In some embodiments, R^{6L3} is —CH₃. In some embodiments, R^{6L3} is —CH₃ and R^{6L4} is C₁₋₆alkyl, C₂₋₆alkynyl, or cycloalkyl. In some embodiments, R^{6L3} is C₁₋₆alkyl and R^{6L4} is C₁₋₆alkyl. In some embodiments, R^{6L3} is H or —CH₃ and R^{6L4} is C₁₋₆alkyl. In some embodiments, R^{6L3} is H or —CH₃ and R^{6L4} is C₂₋₆alkynyl. In some embodiments, R^{6L3} is H or —CH₃ and R^{6L4} is cycloalkyl. In some embodiments, the C₁₋₆alkyl, C₂₋₆alkynyl, or cycloalkyl is unsubstituted or substituted with one or more substituents independently selected from the group consisting of halo, C₁₋₆alkyl, C₁₋₆haloalkyl, cycloalkyl, and halocycloalkyl. In some embodiments, the C₁₋₆alkyl is substituted with halo. In some embodiments, the C₁₋₆alkyl is substituted with cycloalkyl. In some embodiments, R^{6a1}, R^{6a2}, R^{6a3}, or R^{6a4} is

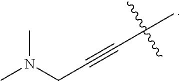 [SMILES: *C#CCN(C)C]

In some embodiments of the compounds of Formula VI and stereoisomers and pharmaceutically acceptable salts thereof, two or more of R^{6a1}, R^{6a2}, R^{6a3}, and R^{6a4} is not H. In some embodiments, R^{6a3} is not H and one of R^{6a1}, R^{6a2}, or R^{6a4} is not H. In some embodiments, R^{6a2} is F or Cl and R^{6a3} is —O—C₁₋₆alkyl or —O-cycloalkyl, wherein the —O—C₁₋₆alkyl or —O-cycloalkyl is unsubstituted or substituted with halo or —CN. In some embodiments, R^{6a1} is not H and R^{6a2} is not H. In some embodiments, R^{6a1} is F or Cl and R^{6a2} is F or Cl.

In some embodiments of the compounds of Formula VI and stereoisomers and pharmaceutically acceptable salts thereof. X³ is —O—. In some embodiments, X³ is —NR^{6b1}—. In some embodiments, X³ is —NH—. In some embodiments, X³ is —N(CH₃)—. In some embodiments, X³ is —CR^{6b2}R^{6b3}—. In some embodiments, X³ is —CH(CH₃)—. In some embodiments, X³ is —CD₂-. In some embodiments, X³ is —CH₂—.

In some embodiments of the compounds of Formula VI and stereoisomers and pharmaceutically acceptable salts thereof. A6 is

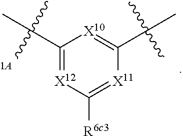

In some embodiments, one or more of X¹⁰, X¹¹, and X¹² is —N—. In some embodiments, X¹⁰ is —N—. In some embodiments, X¹¹ is —N—. In some embodiments, X¹² is —N—. In some embodiments, X¹⁰ is —CR^{6c1}—. In some embodiments, X¹¹ is —CR^{6c2}—. In some embodiments, X¹² is —CR^{6c3}—. In some embodiments, X¹⁰ is —CR^{6c1}—, X¹¹ is —CR^{6c2}—, and X¹² is —CR^{6c3}—.

In some embodiments of the compounds of Formula VI and stereoisomers and pharmaceutically acceptable salts thereof, one or more of R^{6c1}, R^{6c2}, R^{6c3}, and R^{6c4} is not H. In some embodiments, one or more of R^{6c1}, R^{6c2}, R^{6c3}, and R^{6c4} is —CN. In some embodiments, one or more of R^{6c1}, R^{6c2}, R^{6c3}, and R^{6c4} is independently Cl, F, or Br. In some embodiments, two or more of R^{6c1}, R^{6c2}, R^{6c3}, and R^{6c4} are independently Cl, F, or Br. In some embodiments, R^{6c2} is F and R^{6c1}, R^{6c3}, and R^{6c4} are H. In some embodiments, R^{6c1}, R^{6c2}, R^{6c3}, and R^{6c4} are H.

In some embodiments of the compounds of Formula VI and stereoisomers and pharmaceutically acceptable salts thereof. A6 is

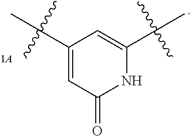 [SMILES: "." ; *c1cc(*)[nH]c(=O)c1]

In some embodiments, A6 is

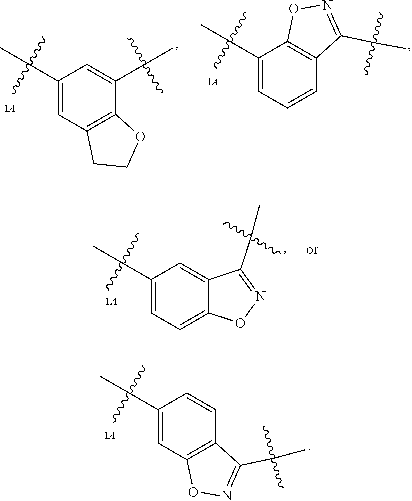 [SMILES: *c1cc(*)c2c(c1)CCO2 ; *c1noc2c(*)cccc12 ; *c1ccc2onc(*)c2c1 ; *c1ccc2c(*)noc2c1]

In some embodiments, A6 is

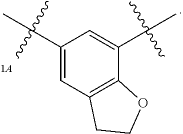 [SMILES: *c1cc(*)c2c(c1)CCO2]

In some embodiments, A6 is

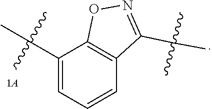 [SMILES: *c1noc2c(*)cccc12]

In some embodiments, A6 is

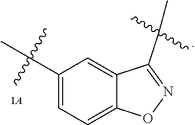 [SMILES: *c1ccc2onc(*)c2c1]

In some embodiments, A6 is

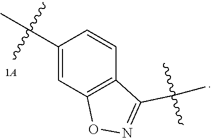 [SMILES: *c1ccc2c(*)noc2c1]

In some embodiments of the compounds of Formula VI and stereoisomers and pharmaceutically acceptable salts thereof, B6 is a 3-membered monocyclic heterocyclediyl, a 4-membered monocyclic heterocyclediyl, a 5-membered monocyclic heterocyclediyl comprising 2 or more N, a 6-membered monocyclic heterocyclediyl comprising 2 or more N, a 7-membered monocyclic heterocyclediyl, an 8-membered monocyclic heterocyclediyl, a 7 to 18-membered polycyclic heterocyclediyl, or a 7 to 18-membered spirocyclic heterocyclediyl, wherein the 3-membered monocyclic heterocyclediyl, 4-membered monocyclic heterocyclediyl, 5-membered monocyclic heterocyclediyl, 6-membered monocyclic heterocyclediyl, 7-membered monocyclic heterocyclediyl, 8-membered monocyclic heterocyclediyl, 7 to 18-membered polycyclic heterocyclediyl, or 7 to 18-membered spirocyclic heterocyclediyl of B6 is unsubstituted or substituted with one or more substituents independently selected from the group consisting of halo, C₁₋₆alkyl, and oxo. In some embodiments, the 7 to 18-membered polycyclic heterocyclediyl is a 7, 8, 9, 10, 11, 12, 13, 14, 15, 16, 17, or 18-membered polycyclic heterocyclediyl. In some embodiments, the 7 to 18-membered spirocyclic heterocyclediyl is a 7, 8, 9, 10, 11, 12, 13, 14, 15, 16, 17, or 18-membered spirocyclic heterocyclediyl.

In some embodiments of the compounds of Formula VI and stereoisomers and pharmaceutically acceptable salts thereof, B6 is a 3 to 8-membered monocyclic heterocyclediyl, wherein the 3 to 8-membered monocyclic heterocyclediyl is unsubstituted or substituted with one or more substituents selected from the group consisting of halo, C₁₋₆alkyl and oxo. In some embodiments, the 3 to 8-membered monocyclic heterocyclediyl is a 3, 4, 5, 6, 7, or 8-membered monocyclic heterocyclediyl. In some embodiments, B6 is a 4-membered monocyclic heterocyclediyl, wherein the 4-membered monocyclic heterocyclediyl is unsubstituted or substituted with one or more substituents selected from the group consisting of halo, C₁₋₆alkyl, and oxo. In some embodiments, B6 is

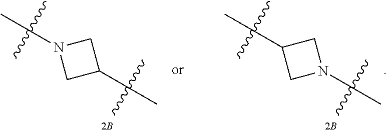 [SMILES: *C1CN(*)C1 ; *C1CN(*)C1]

In some embodiments, B6 is a 6-membered monocyclic heterocyclediyl, wherein the 6-membered monocyclic heterocyclediyl is unsubstituted or substituted with one or more substituents selected from the group consisting of halo, C₁₋₆alkyl, and oxo. In some embodiments, B6 is

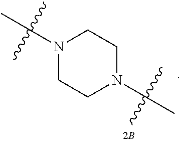 [SMILES: *N1CCN(*)CC1]

In some embodiments, B6 is a 7-membered monocyclic heterocyclediyl, wherein the 7-membered monocyclic heterocyclediyl is unsubstituted or substituted with one or more substituents selected from the group consisting of halo, C₁₋₆alkyl, and oxo. In some embodiments, B6 is

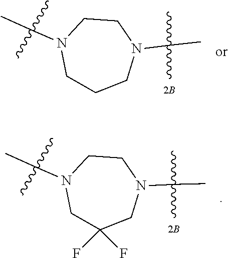 [SMILES: *N1CCCN(*)CC1 ; *N1CCN(*)CC(F)(F)C1]

In some embodiments of the compounds of Formula VI and stereoisomers and pharmaceutically acceptable salts thereof, B6 is a 7 to 18-membered polycyclic heterocyclediyl, wherein the 7 to 18-membered polycyclic heterocyclediyl is unsubstituted or substituted with one or more substituents selected from the group consisting of halo, C₁₋₆alkyl, and oxo. In some embodiments, the 7 to 18-membered polycyclic heterocyclediyl is a 7, 8, 9, 10, 11, 12, 13, 14, 15, 16, 17, or 18-membered polycyclic heterocyclediyl. In some embodiments, B6 is

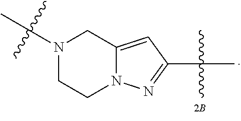 [SMILES: *c1cc2n(n1)CCN(*)C2]

In some embodiments, B6 is

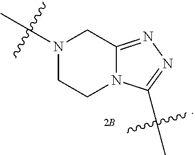 [SMILES: *c1nnc2n1CCN(*)C2]

In some embodiments, B6 is

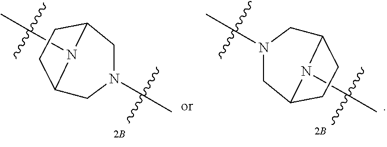 [SMILES: *N1CC2CCC(C1)N2* ; *N1CC2CCC(C1)N2*]

In some embodiments, B6 is

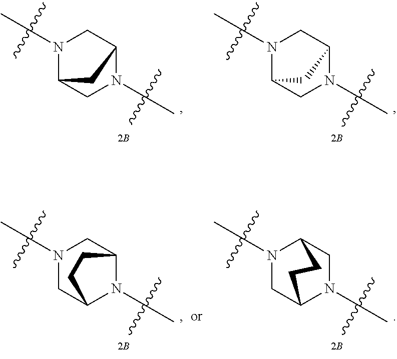

In some embodiments, B6 is

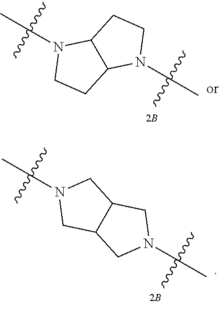 [SMILES: *N1CCC2C1CCN2* ; *N1CC2CN(*)CC2C1]

In some embodiments, B6 is

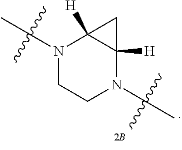 [SMILES: *N1CCN(*)[C@H]2C[C@H]21]

In some embodiments of the compounds of Formula VI and stereoisomers and pharmaceutically acceptable salts thereof, B6 is a 7 to 18-membered spirocyclic heterocyclediyl, wherein the 7 to 18-membered spirocyclic heterocyclediyl is unsubstituted or substituted with one or more substituents selected from the group consisting of halo, C₁₋₆alkyl, and oxo. In some embodiments, the 7 to 18-membered spirocyclic heterocyclediyl is a 7, 8, 9, 10, 11, 12, 13, 14, 15, 16, 17, or 18-membered spirocyclic heterocyclediyl. In some embodiments, B6 is

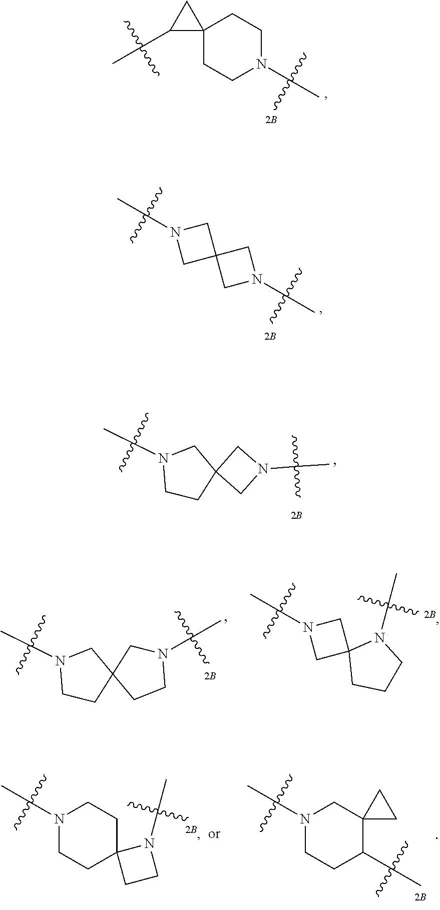

In some embodiments of the compounds of Formula VI and stereoisomers and pharmaceutically acceptable salts thereof, the monocyclic heterocyclediyl, polycyclic heterocyclediyl, or spirocyclic heterocyclediyl of B6 comprises one or more N. In some embodiments, the monocyclic heterocyclediyl, polycyclic heterocyclediyl, or spirocyclic heterocyclediyl of B6 comprises two or more N.

In some embodiments of the compounds of Formula VI and stereoisomers and pharmaceutically acceptable salts thereof, D6 is C₁₋₆alkyl, wherein the C₁₋₆alkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and C(O)N(R^{6D10})₂. In some embodiments, the C₁₋₆alkyl is unsubstituted or substituted with halo. In some embodiments, the C₁₋₆alkyl is unsubstituted or substituted with —OH. In some embodiments, the C₁₋₆alkyl is unsubstituted or substituted with —O—C₁₋₆alkyl.

In some embodiments of the compounds of Formula VI and stereoisomers and pharmaceutically acceptable salts thereof, D6 is cycloalkyl, wherein the cycloalkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{6D10})₂. In some embodiments, the cycloalkyl is unsubstituted or substituted with halo. In some embodiments, D6 is

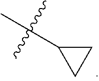 [SMILES: *C1CC1]

In some embodiments of the compounds of Formula VI and stereoisomers and pharmaceutically acceptable salts thereof, D6 is aryl, wherein the aryl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{6D10})₂. In some embodiments, the aryl is a monocyclic 6-membered aryl. In some embodiments, the aryl is substituted with one or more substituents selected from the group consisting of halo, —CN, C₁₋₆alkyl, and C₁₋₆haloalkyl. In some embodiments, the aryl is substituted with —CN.

In some embodiments of the compounds of Formula VI and stereoisomers and pharmaceutically acceptable salts thereof, D6 is heteroaryl, wherein the heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, —C(O)NH₂, —C(O)NHCH₃, and —C(O)N(CH₃)₂.

In some embodiments of the compounds of Formula VI and stereoisomers and pharmaceutically acceptable salts thereof, D6 is a monocyclic 5 or 6-membered heteroaryl comprising one or more N, wherein the monocyclic 5 or 6-membered heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, —C(O)NH₂, —C(O)NHCH₃, and —C(O)N(CH₃)₂. In some embodiments, the monocyclic 5 or 6-membered heteroaryl is substituted with —CN. In some embodiments, the monocyclic 5 or 6-membered heteroaryl is substituted with —CF₃. In some embodiments, the monocyclic 5 or 6-membered heteroaryl is substituted with —OCH₃.

In some embodiments of the compounds of Formula VI and stereoisomers and pharmaceutically acceptable salts thereof, D6 is —O—C₁₋₆alkyl, wherein the —O—C₁₋₆alkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{6D10})₂. In some embodiments, the —O—C₁₋₆alkyl is unsubstituted or substituted with halo.

In some embodiments of the compounds of Formula VI and stereoisomers and pharmaceutically acceptable salts thereof, D6 is —O-aryl or —O-heteroaryl, wherein the —O-aryl or —O-heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{6D10})₂. In some embodiments, the —O-aryl or —O-heteroaryl is unsubstituted or substituted with halo. In some embodiments, the —O-aryl or —O-heteroaryl is unsubstituted or substituted with —CN. In some embodiments, D6 is

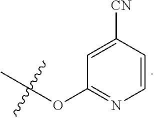 [SMILES: *Oc1cc(C#N)ccn1]

In some embodiments of the compounds of Formula VI and stereoisomers and pharmaceutically acceptable salts thereof, D6 is —C(O)—C₁₋₆alkyl, wherein the —C(O)—C₁₋₆alkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{6D10})₂. In some embodiments, the —C(O)—C₁₋₆alkyl is unsubstituted or substituted with halo. In some embodiments, the —C(O)—C₁₋₆alkyl is unsubstituted or substituted with —CN. In some embodiments, D6 is

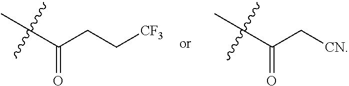 [SMILES: *C(=O)CCC(F)(F)F ; *C(=O)CC#N]

In some embodiments of the compounds of Formula VI and stereoisomers and pharmaceutically acceptable salts thereof, D6 is —C(O)-cycloalkyl, wherein the —C(O)-cycloalkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{6D10})₂. In some embodiments, the —C(O)-cycloalkyl is unsubstituted or substituted with halo. In some embodiments, D6 is

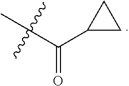 [SMILES: *C(=O)C1CC1]

In some embodiments of the compounds of Formula VI and stereoisomers and pharmaceutically acceptable salts thereof, D6 is —C(O)-heterocyclyl, wherein the —C(O)-heterocyclyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{6D10})₂. In some embodiments, the —C(O)-heterocyclyl is unsubstituted or substituted with halo. In some embodiments, D6 is

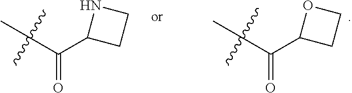 [SMILES: *C(=O)C1CCN1 ; *C(=O)C1CCO1]

In some embodiments of the compounds of Formula VI and stereoisomers and pharmaceutically acceptable salts thereof, D6 is —C(O)-aryl or —C(O)-heteroaryl, wherein the —C(O)-aryl or —C(O)-heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{6D10})₂. In some embodiments, the —C(O)-aryl or —C(O)-heteroaryl is unsubstituted or substituted with halo. In some embodiments, the —C(O)-aryl or —C(O)-heteroaryl is unsubstituted or substituted with —CF₃. In some embodiments, the —C(O)-aryl or —C(O)-heteroaryl is unsubstituted or substituted with —CN. In some embodiments, D6 is

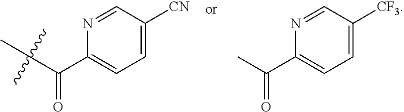

In some embodiments of the compounds of Formula VI and stereoisomers and pharmaceutically acceptable salts thereof, D6 is —N(R^{6D1})(R^{6D2}). In some embodiments, D6 is

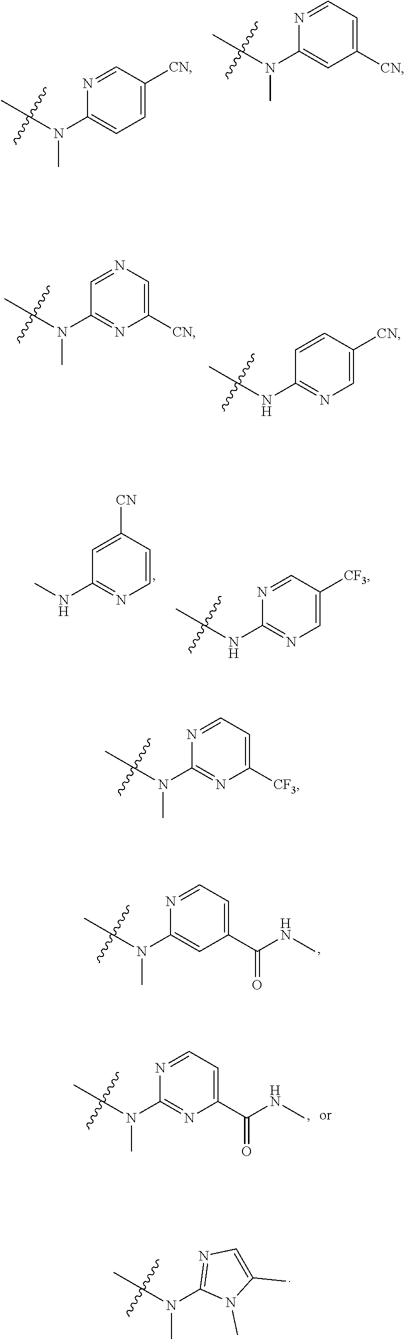

In some embodiments of the compounds of Formula VI and stereoisomers and pharmaceutically acceptable salts thereof, R^{6D1} is H. In some embodiments, R^{6D1} is C₁₋₆alkyl. In some embodiments, R^{6D2} is aryl. In some embodiments, R^{6D2} is heteroaryl. In some embodiments, the heteroaryl is a 6-membered heteroaryl.

In some embodiments of the compounds of Formula VI and stereoisomers and pharmaceutically acceptable salts thereof, D6 is —C(O)N(R^{6D3})(R^{6D4}). In some embodiments, D6 is

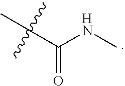

In some embodiments of the compounds of Formula VI and stereoisomers and pharmaceutically acceptable salts thereof, R^{6D3} is H. In some embodiments, R^{6D3} is C₁₋₆alkyl. In some embodiments, R^{6D4} is C₁₋₆alkyl. In some embodiments, R^{6D3} is H and R^{6D4} is C₁₋₆alkyl.

In some embodiments of the compounds of Formula VI and stereoisomers and pharmaceutically acceptable salts thereof, D6 is —N(R^{6D5})C(O)R^{6D6}—. In some embodiments, D6 is

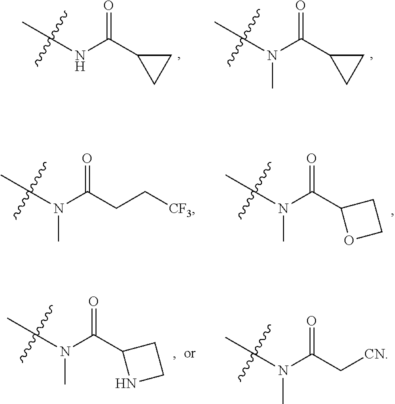

In some embodiments of the compounds of Formula VI and stereoisomers and pharmaceutically acceptable salts thereof, R^{6D5} is H. In some embodiments, R^{6D5} is C₁₋₆alkyl. In some embodiments, R^{6D6} is C₁₋₆alkyl. In some embodiments, R^{6D6} is cycloalkyl. In some embodiments, R^{6D6} is heterocyclyl. In some embodiments, R^{6D5} is H and R^{6D6} is C₁₋₆alkyl, cycloalkyl, or heterocyclyl.

In some embodiments, the compound is of Formula VII:

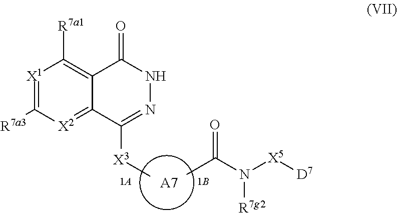

and stereoisomers and pharmaceutically acceptable salts thereof, wherein:
X¹ is —N— or —CR^{7a2}—;
X² is —N— or —CR^{7a4}—;
R^{7a1}, R^{7a2}, R^{7a3}, and R^{7a4} are independently H, halo, —OH, C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, —SO₂-cycloalkyl, —NR^{7L1}R^{7L2}, or C₂₋₆alkynyl-NR^{7L3}R^{7L4};
wherein if X¹ is —CR^{7a2}— and X² is —CR^{7a4}—, then at least one of R^{7a1}, R^{7a2}, R^{7a3}, and R^{7a4} is not H;
wherein each C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, or —SO₂-cycloalkyl of R^{7a1}, R^{7a2}, R^{7a3}, and R^{7a4} is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂;
R^{7L1}, R^{7L2}, R^{7L3}, and R^{7L4} are independently H, C₁₋₆alkyl, C₂₋₆alkynyl, or cycloalkyl, wherein each C₁₋₆alkyl, C₂₋₆alkynyl, or cycloalkyl of R^{7L1}, R^{7L2}, R^{7L3}, and R^{7L4} is unsubstituted or substituted with one or more substituents independently selected from the group consisting of halo, C₁₋₆alkyl, C₁₋₆haloalkyl, cycloalkyl, and halocycloalkyl;
X³ is —O—, —NR^{7b1}, or —CR^{7b2}R^{7b3}—, wherein R^{7b1}, R^{7b2}, and R^{7b3} are independently H or C₁₋₆alkyl;
A7 is

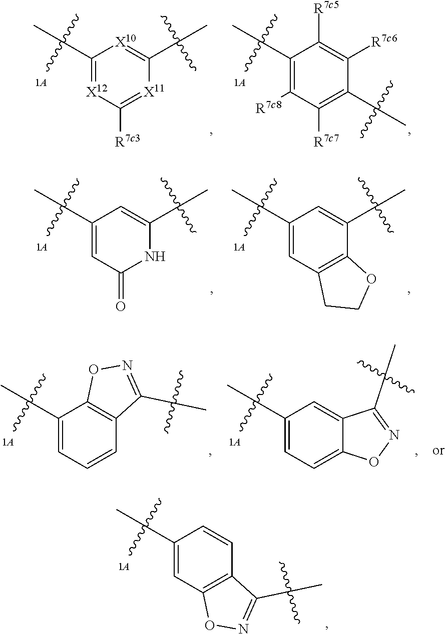

wherein a bond marked 1A is to X³;
X¹⁰ is —N— or —CR^{7c1}—, X¹¹ is —N— or —CR^{7c2}—, and X¹² is —N— or CR^{7c4}—;
R^{7c1}, R^{7c2}, R^{7c3}, R^{7c4}, R^{7c5}, R^{7c6}, R^{7c7}, and R^{7c8} are independently H, halo, —CN, —SO₂CH₃, —SO₂NH₂, or —NHSO₂CH₃;
X⁵ is —CH(R^{7g3})— or —CH(R^{7g4})CH₂N(R^{7g5})—;
R^{7g2}, R^{7g3}, R^{7g4}, and R^{7g5} are independently H or C₁₋₆alkyl;
D7 is C₁₋₆alkyl, cycloalkyl, aryl, heteroaryl, —O—C₁₋₆alkyl, —O-aryl, —O-heteroaryl, —C(O)—C₁₋₆alkyl, —(O)-cycloalkyl, —C(O)-heterocyclyl, —C(O)-aryl, —C(O)-heteroaryl, —N(R^{7D1})(R^{7D2}), —C(O)N(R^{7D3})(R^{7D4}), or —N(R^{7D5})C(O)R^{7D6};
wherein the C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, or —C(O)-heteroaryl of D7 is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-C₁₋₆alkyl, C₁₋₆alkyl-O—C₁₋₆haloalkyl, —C(O)-cycloalkyl, and —C(O)N(R^{7D10})₂, wherein each R^{7D10} is independently H or C₁₋₆alkyl;
R^{7D1}, R^{7D3}, and R^{7D5} are independently H or C₁₋₆alkyl;
R^{7D2} is aryl or heteroaryl, wherein the aryl or heteroaryl of R^{7D2} is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{7D11})₂, wherein each R^{7D11} is independently H or C₁₋₆alkyl; and
R^{7D4} and R^{7D6} are independently C₁₋₆alkyl, cycloalkyl, heterocyclyl, aryl, or heteroaryl, wherein each C₁₋₆alkyl, cycloalkyl, heterocyclyl, aryl, or heteroaryl of R^{7D4} and R^{7D6} is unsubstituted or substituted with one or more substituents independently selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{7D12})₂, wherein each R^{7D12} is independently H or C₁₋₆alkyl.

In some embodiments of the compounds of Formula VII and stereoisomers and pharmaceutically acceptable salts thereof. X¹ is —CR^{7a2}—. In some embodiments, X² is —CR^{7a4}—. In some embodiments, X¹ is —N—. In some embodiments, X² is —N—. In some embodiments, X¹ is —CR^{7a2}— and X² is —CR^{7a4}—. In some embodiments, X¹ is —CR^{7a2}— and X² is —N—. In some embodiments, X¹ is —N— and X² is —CR^{7a4}—.

In some embodiments of the compounds of Formula VII and stereoisomers and pharmaceutically acceptable salts thereof, one or more of R^{7a1} is not H. In some embodiments, R^{7a2} is not H. In some embodiments, R^{7a3} is not H. In some embodiments, R^{7a4} is not H.

In some embodiments of the compounds of Formula VII and stereoisomers and pharmaceutically acceptable salts thereof, one or more of R^{7a1}, R^{7a2}, R^{7a3}, and R^{7a4} is halo. In some embodiments, R^{7a1} is halo. In some embodiments, R^{7a2} is halo. In some embodiments, R^{7a3} is halo. In some embodiments, R^{7a4} is halo. In some embodiments, the halo is F, Cl, or Br. In some embodiments, the halo is Cl. In some embodiments, the halo is F. In some embodiments, R^{7a1} is F. In some embodiments, R^{7a3} is F. In some embodiments, R^{7a3} is F.

In some embodiments of the compounds of Formula VII and stereoisomers and pharmaceutically acceptable salts thereof, one or more of R^{7a1}, R^{7a2}, R^{7a3}, and R^{7a4} is —OH. In some embodiments, R^{7a1} is —OH. In some embodiments, R^{7a2} is —OH. In some embodiments, R^{7a3} is —OH. In some embodiments, R^{7a4} is —OH.

In some embodiments of the compounds of Formula VII and stereoisomers and pharmaceutically acceptable salts thereof, one or more of R^{7a1}, R^{7a2}, R^{7a3}, and R^{7a4} is C₂₋₆alkenyl. In some embodiments, R^{7a1} is C₂₋₆alkenyl. In some embodiments, R^{7a2} is C₂₋₆alkenyl. In some embodiments, R^{7a3} is C₂₋₆alkenyl. In some embodiments, R^{7a4} is C₂₋₆alkenyl. In some embodiments, the C₂₋₆alkenyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the C₂₋₆alkenyl is substituted with halo. In some embodiments, R^{7a1}, R^{7a2}, R^{7a3}, or R^{7a4} is

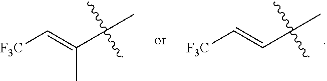

In some embodiments of the compounds of Formula VII and stereoisomers and pharmaceutically acceptable salts thereof, one or more of R^{7a1}, R^{7a2}, R^{7a3}, and R^{7a4} is C₂₋₆alkynyl. In some embodiments, R^{7a1} is C₂₋₆alkynyl. In some embodiments, R^{7a2} is C₂₋₆alkynyl. In some embodiments, R^{7a3} is C₂₋₆alkynyl. In some embodiments, R^{7a4} is C₂₋₆alkynyl. In some embodiments, the C₂₋₆alkynyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the C₂₋₆alkynyl is substituted with halo. In some embodiments, the C₂₋₆alkynyl is substituted with cycloalkyl. In some embodiments, R^{7a1}, R^{7a2}, R^{7a3}, or R^{7a4} is

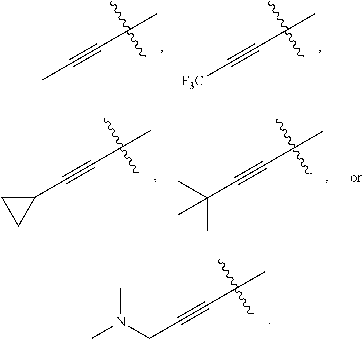

In some embodiments of the compounds of Formula VII and stereoisomers and pharmaceutically acceptable salts thereof, one or more of R^{7a1}, R^{7a2}, R^{7a3}, and R^{7a4} is cycloalkyl. In some embodiments, R^{7a1} is cycloalkyl. In some embodiments, R^{7a2} is cycloalkyl. In some embodiments, R^{7a3} is cycloalkyl. In some embodiments, R^{7a4} is cycloalkyl. In some embodiments, the cycloalkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the cycloalkyl is substituted with halo. In some embodiments, R^{7a1}, R^{7a2}, R^{7a3}, or R^{7a4} is

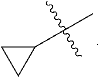 [SMILES: *C1CC1]

In some embodiments of the compounds of Formula VII and stereoisomers and pharmaceutically acceptable salts thereof, one or more of R^{7a1}, R^{7a2}, R^{7a3}, and R^{7a4} is heterocyclyl. In some embodiments, R^{7a1} is heterocyclyl. In some embodiments, R^{7a2} is heterocyclyl. In some embodiments, R^{7a3} is heterocyclyl. In some embodiments, R^{7a4} is heterocyclyl. In some embodiments, the heterocyclyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the heterocyclyl is substituted with halo. In some embodiments, the heterocyclyl is substituted with —OH. In some embodiments, R^{7a1}, R^{7a2}, R^{7a3}, or R^{7a4} is

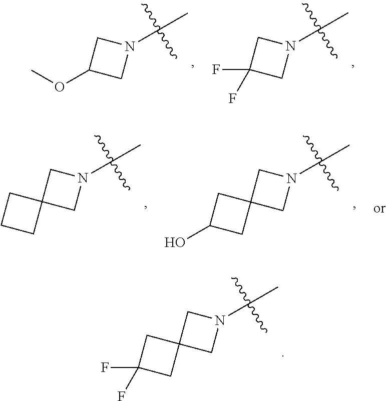

In some embodiments of the compounds of Formula VII and stereoisomers and pharmaceutically acceptable salts thereof, one or more of R^{7a1}, R^{7a2}, R^{7a3}, and R^{7a4} is aryl or heteroaryl. In some embodiments, R^{7a1} is aryl or heteroaryl. In some embodiments, R^{7a2} is aryl or heteroaryl. In some embodiments, R^{7a3} is aryl or heteroaryl. In some embodiments, R^{7a4} is aryl or heteroaryl. In some embodiments, the aryl or heteroaryl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, R^{7a1}, R^{7a2}, R^{7a3}, or R^{7a4} is

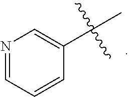 [SMILES: *c1cccnc1]

In some embodiments of the compounds of Formula VII and stereoisomers and pharmaceutically acceptable salts thereof, one or more of R^{7a1}, R^{7a2}, R^{7a3}, and R^{7a4} is —O—C₁₋₆alkyl. In some embodiments, R^{7a1} is —O—C₁₋₆alkyl. In some embodiments, R^{7a2} is —O—C₁₋₆alkyl. In some embodiments, R^{7a3} is —O—C₁₋₆alkyl. In some embodiments, R^{7a4} is —O—C₁, alkyl. In some embodiments, the —O—C₁₋₆alkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O—C₁₋₆alkyl is substituted with halo. In some embodiments, the —O—C₁₋₆alkyl is substituted with cycloalkyl. In some embodiments, R^{7a1}, R^{7a2}, R^{7a3}, or R^{7a4} is —OCH₃, —OCHF₂, —OCH₂CF₃, or

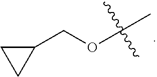 [SMILES: *OCC1CC1]

In some embodiments of the compounds of Formula VII and stereoisomers and pharmaceutically acceptable salts thereof, one or more of R^{7a1}, R^{7a2}, R^{7a3}, and R^{7a4} is —O—C₂₋₆alkenyl. In some embodiments, R^{7a1} is —O—C₂₋₆alkenyl. In some embodiments, R^{7a2} is —O—C₂₋₆alkenyl. In some embodiments, R^{7a3} is —O—C₂₋₆alkenyl. In some embodiments, R^{7a4} is —O—C₂₋₆alkenyl. In some embodiments, the —O—C₂₋₆alkenyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O—C₂₋₆alkenyl is substituted with halo.

In some embodiments of the compounds of Formula VII and stereoisomers and pharmaceutically acceptable salts thereof, one or more of R^{7a1}, R^{7a2}, R^{7a3}, and R^{7a4} is —O—C₂₋₆alkynyl. In some embodiments, R^{7a1} is —O—C₂₋₆alkynyl. In some embodiments, R^{7a2} is —O—C₂₋₆alkynyl. In some embodiments, R^{7a3} is —O—C₂₋₆alkynyl. In some embodiments, R^{7a4} is —O—C₂₋₆alkynyl. In some embodiments, the O—C₂₋₆alkynyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O—C₂₋₆alkynyl is substituted with halo. In some embodiments, R^{7a1}, R^{7a2}, R^{7a3}, or R^{7a4} is

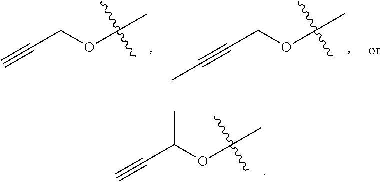

In some embodiments of the compounds of Formula VII and stereoisomers and pharmaceutically acceptable salts thereof, one or more of R^{7a1}, R^{7a2}, R^{7a3}, and R^{7a4} is —O-cycloalkyl. In some embodiments, R^{7a1} is —O-cycloalkyl. In some embodiments, R^{7a2} is —O-cycloalkyl. In some embodiments, R^{7a3} is —O-cycloalkyl. In some embodiments, R^{7a4} is cycloalkyl. In some embodiments, the —O-cycloalkyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-cycloalkyl is substituted with halo. In some embodiments, the —O-cycloalkyl is substituted with C₁₋₆haloalkyl. In some embodiments, R^{7a1}, R^{7a2}, R^{7a3}, or R^{7a4} is

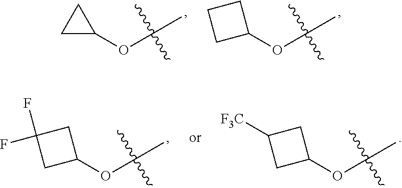 [SMILES: *OC1CC1 ; *OC1CCC1 ; *OC1CC(F)(F)C1 ; *OC1CC(C(F)(F)F)C1]

In some embodiments of the compounds of Formula VII and stereoisomers and pharmaceutically acceptable salts thereof, one or more of R^{7a1}, R^{7a2}, R^{7a3}, and R^{7a4} is —O-heterocyclyl. In some embodiments, R^{7a1} is —O-heterocyclyl. In some embodiments, R^{7a2} is —O-heterocyclyl. In some embodiments, R^{7a3} is —O-heterocyclyl. In some embodiments, R^{7a4} is —O-heterocyclyl. In some embodiments, the —O-heterocyclyl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-heterocyclyl is substituted with halo.

In some embodiments of the compounds of Formula VII and stereoisomers and pharmaceutically acceptable salts thereof, one or more of R^{7a1}, R^{7a2}, R^{7a3}, and R^{7a4} is —O-aryl. In some embodiments, R^{7a1} is —O-aryl. In some embodiments, R^{7a2} is —O-aryl. In some embodiments, R^{7a3} is —O-aryl. In some embodiments, R^{7a4} is —O-aryl. In some embodiments, the —O-aryl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-aryl is substituted with halo. In some embodiments, the —O-aryl is substituted with C₁₋₆alkyl. In some embodiments, the —O-aryl is substituted with —C(O)NH₂. In some embodiments, the aryl of is a 6 membered monocyclic aryl. In some embodiments, R^{7a3} is —O-aryl wherein the aryl is an unsubstituted 6-membered monocyclic aryl. In some embodiments, R^{7a1}, R^{7a2}, R^{7a3}, or R^{7a4} is

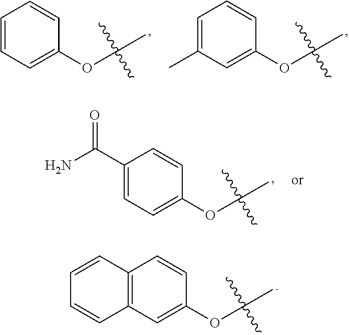

In some embodiments of the compounds of Formula VII and stereoisomers and pharmaceutically acceptable salts thereof, one or more of R^{7a1}, R^{7a2}, R^{7a3}, and R^{7a4} is —O-heteroaryl. In some embodiments, R^{7a1} is —O-heteroaryl. In some embodiments, R^{7a2} is —O-heteroaryl. In some embodiments, R^{7a3} is —O-heteroaryl. In some embodiments, R^{7a4} is —O-heteroaryl. In some embodiments, the —O-heteroaryl is unsubstituted or substituted with one or more substituents independently selected from the group consisting of halo, —OH, C₁₋₆alkyl, C₁₋₆haloalkyl, cycloalkyl, halocycloalkyl, —O—C₁₋₆alkyl, and —C(O)NH₂. In some embodiments, the —O-heteroaryl is substituted with halo. In some embodiments, R^{7a1}, R^{7a2}, R^{7a3}, or R^{7a1} is

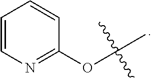 [SMILES: *Oc1ccccn1]

In some embodiments of the compounds of Formula VII and stereoisomers and pharmaceutically acceptable salts thereof, one or more of R^{7a1}, R^{7a2}, R^{7a3}, and R^{7a4} is —SO₂-cycloalkyl. In some embodiments, R^{7a1} is —SO₂-cycloalkyl. In some embodiments, R^{7a2} is —SO₂-cycloalkyl. In some embodiments, R^{7a3} is —SO₂-cycloalkyl. In some embodiments, R^{7a4} is —SO₂-cycloalkyl. In some embodiments, the —SO₂-cycloalkyl is unsubstituted or substituted with one or more substituents independently selected from the group consisting of halo, —OH, C₁₋₆alkyl, C₁₋₆-haloalkyl, cycloalkyl, halocycloalkyl, —O—C₁₋₆alkyl, and —C(O)NH₂. In some embodiments, the —SO₂-cycloalkyl is substituted with halo. In some embodiments, R^{7a1}, R^{7a2}, R^{7a3}, or R^{7a4} is

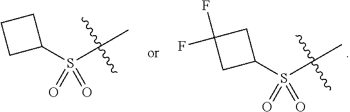 [SMILES: *S(=O)(=O)C1CCC1 ; *S(=O)(=O)C1CC(F)(F)C1]

In some embodiments of the compounds of Formula VII and stereoisomers and pharmaceutically acceptable salts thereof, one or more of R^{7a1}, R^{7a2}, R^{7a3}, and R^{7a4} is —NR^{7L1}R^{7L2}. In some embodiments, R^{7a1} is —NR^{7L1}R^{7L2}. In some embodiments, R^{7a2} is —NR^{7L1}R^{7L2}. In some embodiments, R^{7a3} is —NR^{7L1}R^{7L2}. In some embodiments, R^{7a4} is —NR^{7L1}R^{7L2}. In some embodiments, R^{7a1}, R^{7a2}, R^{7a3}, or R^{7a4} is

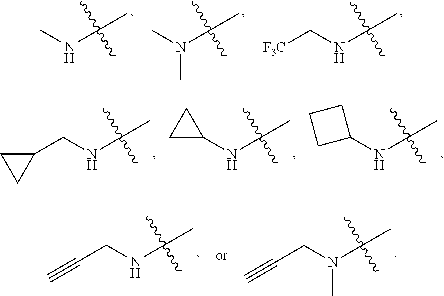

In some embodiments of the compounds of Formula VII and stereoisomers and pharmaceutically acceptable salts thereof, R^{7L1} and R^{7L2} are independently H, C₁₋₆alkyl, C₂₋₆alkynyl, or cycloalkyl. In some embodiments, R^{7L1} is H. In some embodiments, R^{7L1} is H and R^{7L2} is C₁₋₆alkyl, C₂₋₆alkynyl, or cycloalkyl. In some embodiments, R^{7L1} is —CH₃. In some embodiments, R^{7L1} is —CH₃ and R^{7L2} is C₁₋₆alkyl, C₂₋₆alkynyl, or cycloalkyl. In some embodiments, R^{7L1} is H or —CH₃ and R^{7L2} is C₁₋₆alkyl. In some embodiments, R^{7L1} is H or —CH₃ and R^{7L2} is C₂₋₆alkynyl. In some embodiments, R^{7L1} is H or —CH₃ and R^{7L2} is cycloalkyl. In some embodiments, the C₁₋₆alkyl, C₁₋₆alkynyl, or cycloalkyl is unsubstituted or substituted with one or more substituents independently selected from the group consisting of halo, C₁₋₆alkyl, C₁₋₆haloalkyl, cycloalkyl, and halocycloalkyl. In some embodiments, the C₁₋₆alkyl is substituted with halo. In some embodiments, the C₁₋₆alkyl is substituted with cycloalkyl.

In some embodiments of the compounds of Formula VII and stereoisomers and pharmaceutically acceptable salts thereof, one or more of R^{7a1}, R^{7a2}, R^{7a3}, and R^{7a4} is C₂₋₆alkynyl-NR^{7L3}R^{7L4}. In some embodiments, R^{7a1} is C₂₋₆alkynyl-NR^{7L3}R^{7L4}. In some embodiments, R is C₂₋₆alkynyl-NR^{7L3}R^{7L4}. In some embodiments, R^{7a3} is C₂₋₆alkynyl-NR^{7L3}R^{7L4}. In some embodiments, R^{7a4} is C₂₋₆alkynyl-NR^{7L3}R^{7L4}.

In some embodiments of the compounds of Formula VII and stereoisomers and pharmaceutically acceptable salts thereof, R^{7L3} and R^{7L4} are independently H, C₁₋₆alkyl, C₂₋₆alkynyl, or cycloalkyl. In some embodiments, R^{7L3} is H. In some embodiments, R^{7L3} is H and R^{7L4} is C₁₋₆alkyl, C₂₋₆alkynyl, or cycloalkyl. In some embodiments, R^{7L3} is —CH₃. In some embodiments, R^{7L3} is —CH₃ and R^{7L4} is C₁₋₆alkyl, C₂₋₆alkynyl, or cycloalkyl. In some embodiments, R^{7L3} is C₁₋₆alkyl and R^{7L4} is C₁₋₆alkyl. In some embodiments, R^{7L3} is H or —CH₃ and R^{7L4} is C₁₋₆alkyl. In some embodiments, R^{7L3} is H or —CH₃ and R^{7L4} is C₂₋₆alkynyl. In some embodiments, R^{7L3} is H or —CH₃ and R^{7L4} is cycloalkyl. In some embodiments, the C₁₋₆alkyl, C₂₋₆alkynyl, or cycloalkyl is unsubstituted or substituted with one or more substituents independently selected from the group consisting of halo, C₁₋₆alkyl, C₁₋₆haloalkyl, cycloalkyl, and halocycloalkyl. In some embodiments, the C₁₋₆alkyl is substituted with halo. In some embodiments, the C₁₋₆alkyl is substituted with cycloalkyl. In some embodiments, R^{7a1}, R^{7a2}, R^{7a3}, or R^{7a4} is

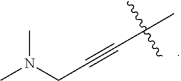 [SMILES: *C#CCN(C)C]

In some embodiments of the compounds of Formula VII and stereoisomers and pharmaceutically acceptable salts thereof, two or more of R^{7a1}, R^{7a2}, R^{7a3}, and R^{7a4} is not H. In some embodiments, R^{7a3} is not H and one of R^{7a1}, R^{7a2}, or R^{7a4} is not H. In some embodiments, R^{7a2} is F or Cl and R^{7a3} is —O—C₁₋₆alkyl or —O-cycloalkyl, wherein the —O—C₁₋₆alkyl or —O-cycloalkyl is unsubstituted or substituted with halo or —CN. In some embodiments, R^{7a1} is not H and R^{7a2} is not H. In some embodiments, R^{7a1} is F or Cl and R^{7a2} is F or Cl.

In some embodiments of the compounds of Formula VII and stereoisomers and pharmaceutically acceptable salts thereof, X³ is —O—. In some embodiments, X³ is —NR^{7b1}—. In some embodiments, X³ is —NH—. In some embodiments, X is —N(CH₃)—. In some embodiments, X³ is —CR^{7b2}R^{7b3}—. In some embodiments, X³ is —CH(CH₃)—. In some embodiments, X³ is —CD₂-. In some embodiments, X³ is —CH₂—.

In some embodiments of the compounds of Formula VII and stereoisomers and pharmaceutically acceptable salts thereof, A7 is

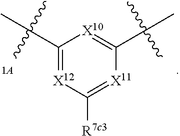

In some embodiments, one or more of X¹⁰, X¹¹, and X¹² is —N—. In some embodiments, X¹⁰ is —N—. In some embodiments, X¹¹ is —N—. In some embodiments, X¹² is —N—. In some embodiments, X¹⁰ is —CR^{7c1}—. In some embodiments, X¹¹ is —CR^{7c2}—. In some embodiments, X¹² is —CR^{7c3}—. In some embodiments, X¹⁰ is —CR^{7c1}—, X¹¹ is —CR^{7c2}—, and X¹² is —CR^{7c3}—.

In some embodiments of the compounds of Formula VII and stereoisomers and pharmaceutically acceptable salts thereof, one or more of R^{7c1}, R^{7c2}, R^{7c3}, and R^{7c4} is not H. In some embodiments, one or more of R^{7c1}, R^{7c2}, R^{7c3}, and R^{7c4} is —CN. In some embodiments, one or more of R^{7c1}, R^{7c2}, R^{7c3}, and R^{7c4} is independently Cl, F, or Br. In some embodiments, two or more of R^{7c1}, R^{7c2}, R^{7c3}, and R^{7c4} are independently Cl, F, or Br. In some embodiments, R^{7c2} is F and R^{7c1}, R^{7c3}, and R^{7c4} are H. In some embodiments, R^{7c1}, R^{7c2}, R^{7c3}, and R^{7c4} are H.

In some embodiments of the compounds of Formula VII and stereoisomers and pharmaceutically acceptable salts thereof, A7 is

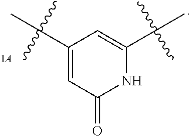 [SMILES: "." ; *c1cc(*)[nH]c(=O)c1]

In some embodiments, A7 is

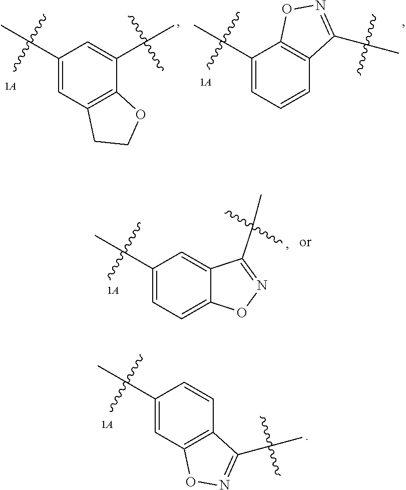 [SMILES: *c1cc(*)c2c(c1)CCO2 ; *c1noc2c(*)cccc12 ; *c1ccc2onc(*)c2c1 ; "," ; "or" ; *c1ccc2c(*)noc2c1]

In some embodiments, A7 is

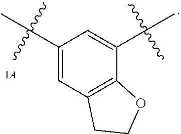 [SMILES: *c1cc(*)c2c(c1)CCO2]

In some embodiments, A7 is

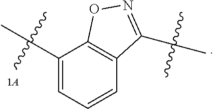 [SMILES: *c1noc2c(*)cccc12 ; "."]

In some embodiments, A7 is

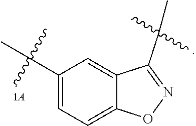 [SMILES: *c1ccc2onc(*)c2c1 ; "."]

In some embodiments, A7 is

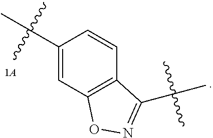 [SMILES: *c1ccc2c(*)noc2c1]

In some embodiments of the compounds of Formula VII and stereoisomers and pharmaceutically acceptable salts thereof, R^{7g2} is H. In some embodiments, R^{7g2} is C₁₋₆ alkyl. In some embodiments, R^{7g2} is —CH₃. In some embodiments, R^{7g2} is —CH₂CH₃.

In some embodiments of the compounds of Formula VII and stereoisomers and pharmaceutically acceptable salts thereof. X⁵ is —CH(R^{7g3})—, wherein R^{7g3} is H or C₁₋₆alkyl. In some embodiments, X⁵ is —CH₂—. In some embodiments, X⁵ is —CH(CH₃)—.

In some embodiments of the compounds of Formula VII and stereoisomers and pharmaceutically acceptable salts thereof. X⁵ is —CH(R^{7g4})CH₂N(R^{7g5})—, wherein R^{7g4} and R^{7g5} are independently H or C₁₋₆alkyl. In some embodiments, R^{7g4} is H and R^{7g5} is C₁₋₆alkyl. In some embodiments, R^{7g5} is —CH₃. In some embodiments, R^{7g5} is —CH₂CH₃. In some embodiments, R^{7g4} is H and R^{7g5} is H.

In some embodiments of the compounds of Formula VII and stereoisomers and pharmaceutically acceptable salts thereof, D7 is C₁₋₆alkyl, wherein the C₁₋₆alkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{7D10})₂. In some embodiments, the C₁₋₆alkyl is unsubstituted or substituted with halo. In some embodiments, the C₁₋₆alkyl is unsubstituted or substituted with —OH. In some embodiments, the C₁₋₆alkyl is unsubstituted or substituted with —O—C₁₋₆alkyl.

In some embodiments of the compounds of Formula VII and stereoisomers and pharmaceutically acceptable salts thereof, D7 is cycloalkyl, wherein the cycloalkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{7D10})₂. In some embodiments, the cycloalkyl is unsubstituted or substituted with halo. In some embodiments, D7 is

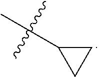 [SMILES: *C1CC1]

In some embodiments of the compounds of Formula VII and stereoisomers and pharmaceutically acceptable salts thereof, D7 is aryl, wherein the aryl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{7D10})₂. In some embodiments, the aryl is a monocyclic 6-membered aryl. In some embodiments, the aryl is substituted with one or more substituents selected from the group consisting of halo, —CN, C₁₋₆alkyl, and C₁₋₆haloalkyl. In some embodiments, the aryl is substituted with —CN.

In some embodiments of the compounds of Formula VII and stereoisomers and pharmaceutically acceptable salts thereof, D7 is heteroaryl, wherein the heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, —C(O)NH₂, —C(O)NHCH₃, and —C(O)N(CH₃)₂.

In some embodiments of the compounds of Formula VII and stereoisomers and pharmaceutically acceptable salts thereof, D7 is a monocyclic 5 or 6-membered heteroaryl comprising one or more N, wherein the monocyclic 5 or 6-membered heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, —C(O)NH₂, —C(O)NHCH₃, and —C(O)N(CH₃)₂. In some embodiments, the monocyclic 5 or 6-membered heteroaryl is substituted with —CN. In some embodiments, the monocyclic 5 or 6-membered heteroaryl is substituted with —CF₃. In some embodiments, the monocyclic 5 or 6-membered heteroaryl is substituted with —OCH₃.

In some embodiments of the compounds of Formula VII and stereoisomers and pharmaceutically acceptable salts thereof, D7 is —O—C₁₋₆alkyl, wherein the —O—C₁₋₆alkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{7D10})₂. In some embodiments, the —O—C₁₋₆alkyl is unsubstituted or substituted with halo.

In some embodiments of the compounds of Formula VII and stereoisomers and pharmaceutically acceptable salts thereof, D7 is —O-aryl or —O-heteroaryl, wherein the —O-aryl or —O-heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{7D10})₂. In some embodiments, the —O-aryl or —O-heteroaryl is unsubstituted or substituted with halo. In some embodiments, the —O-aryl or —O-heteroaryl is unsubstituted or substituted with —CN. In some embodiments, D7 is

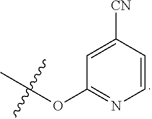 [SMILES: *Oc1cc(C#N)ccn1 ; "."]

In some embodiments of the compounds of Formula VII and stereoisomers and pharmaceutically acceptable salts thereof, D7 is —C(O)—C₁₋₆alkyl, wherein the —C(O)—C₁₋₆alkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{7D10})₂. In some embodiments, the —C(O)—C₁₋₆alkyl is unsubstituted or substituted with halo. In some embodiments, the —C(O)—C₁₋₆alkyl is unsubstituted or substituted with —CN. In some embodiments, D7 is

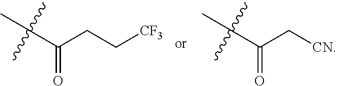 [SMILES: *C(=O)CCC(F)(F)F ; *C(=O)CC#N]

In some embodiments of the compounds of Formula VII and stereoisomers and pharmaceutically acceptable salts thereof, D7 is —C(O)-cycloalkyl, wherein the —C(O)-cycloalkyl is unsubstituted or substituted with one or more substituents selected from the group consisting of halo, —CN, C₁₋₆alkyl, C₁₋₆haloalkyl, cycloalkyl, —OH, —C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{7D10}). In some embodiments, the —C(O)-cycloalkyl is unsubstituted or substituted with halo. In some embodiments, D7 is

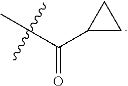 [SMILES: *C(=O)C1CC1]

In some embodiments of the compounds of Formula VII and stereoisomers and pharmaceutically acceptable salts thereof, D7 is —C(O)-heterocyclyl, wherein the —C(O)-heterocyclyl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{7D10})₂. In some embodiments, the —C(O)-heterocyclyl is unsubstituted or substituted with halo. In some embodiments, D7 is

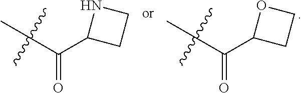 [SMILES: *C(=O)C1CCN1 ; "or" ; *C(=O)C1CCO1 ; "."]

In some embodiments of the compounds of Formula VII and stereoisomers and pharmaceutically acceptable salts thereof, D7 is —C(O)-aryl or —C(O)-heteroaryl, wherein the —C(O)-aryl or —C(O)-heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{7D10})₂. In some embodiments, the —C(O)-aryl or —C(O)-heteroaryl is unsubstituted or substituted with halo. In some embodiments, the —C(O)-aryl or —C(O)-heteroaryl is unsubstituted or substituted with —CF₃. In some embodiments, the —C(O)-aryl or —C(O)-heteroaryl is unsubstituted or substituted with —CN. In some embodiments, D7 is

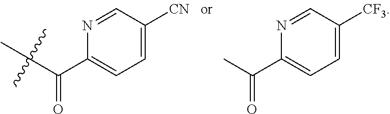

In some embodiments of the compounds of Formula VII and stereoisomers and pharmaceutically acceptable salts thereof, D7 is —N(R^{7D1})(R^{7D2}). In some embodiments, D7 is

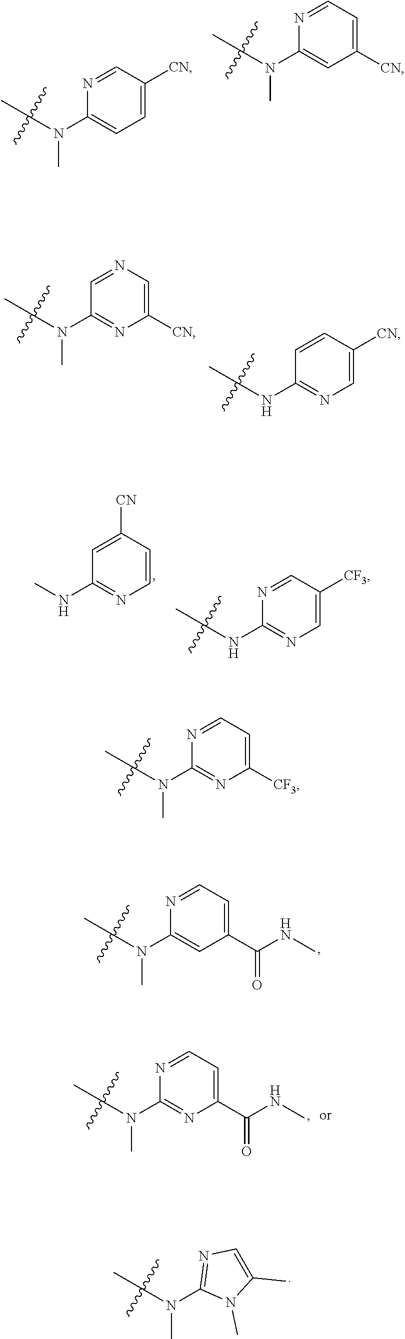

In some embodiments of the compounds of Formula VII and stereoisomers and pharmaceutically acceptable salts thereof, R^{7D1} is H. In some embodiments, R^{7D1} is C₁₋₆alkyl. In some embodiments, R^{7D2} is aryl. In some embodiments, R^{7D2} is heteroaryl. In some embodiments, the heteroaryl is a 6-membered heteroaryl.

In some embodiments of the compounds of Formula VII and stereoisomers and pharmaceutically acceptable salts thereof, D7 is —C(O)N(R^{7D3})(R^{7D4}). In some embodiments, D7 is

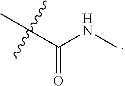

In some embodiments of the compounds of Formula VII and stereoisomers and pharmaceutically acceptable salts thereof, R^{7D3} is H. In some embodiments, R^{7D3} is C₁₋₆alkyl. In some embodiments, R^{7D4} is C₁₋₆alkyl. In some embodiments, R^{7D3} is H and R^{7D4} is C₁₋₆alkyl.

In some embodiments of the compounds of Formula VII and stereoisomers and pharmaceutically acceptable salts thereof, D7 is —N(R^{7D5})C(O)R^{7D6}—. In some embodiments D7 is

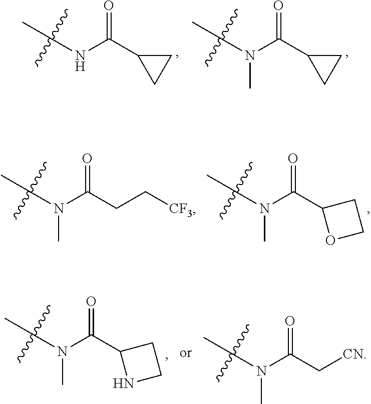

In some embodiments of the compounds of Formula VII and stereoisomers and pharmaceutically acceptable salts thereof, R^{7D5} is H. In some embodiments, R^{7D5} is C₁₋₆alkyl. In some embodiments, R^{7D6} is C₁₋₆alkyl. In some embodiments, R^{7D6} is cycloalkyl. In some embodiments, R^{7D6} is heterocyclyl. In some embodiments, R^{7D5} is H and R^{7D6} is C₁₋₆alkyl, cycloalkyl, or heterocyclyl.

In some embodiments, the compound, and stereoisomers and pharmaceutically acceptable salt thereof, is selected from the group consisting of

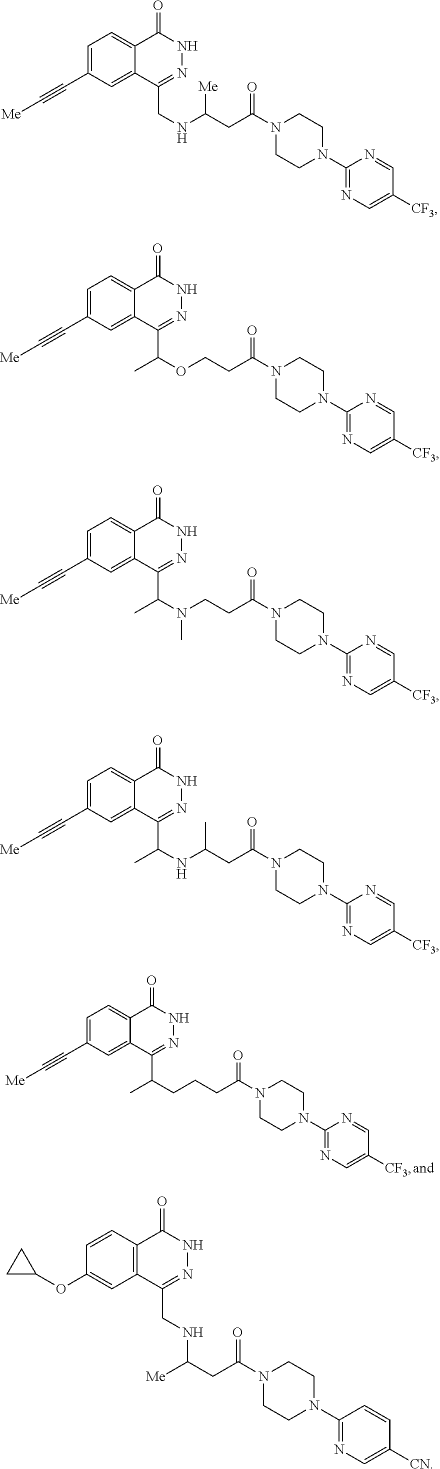

In some embodiments, the compound, and stereoisomers and pharmaceutically acceptable salt thereof, is selected from the group consisting of

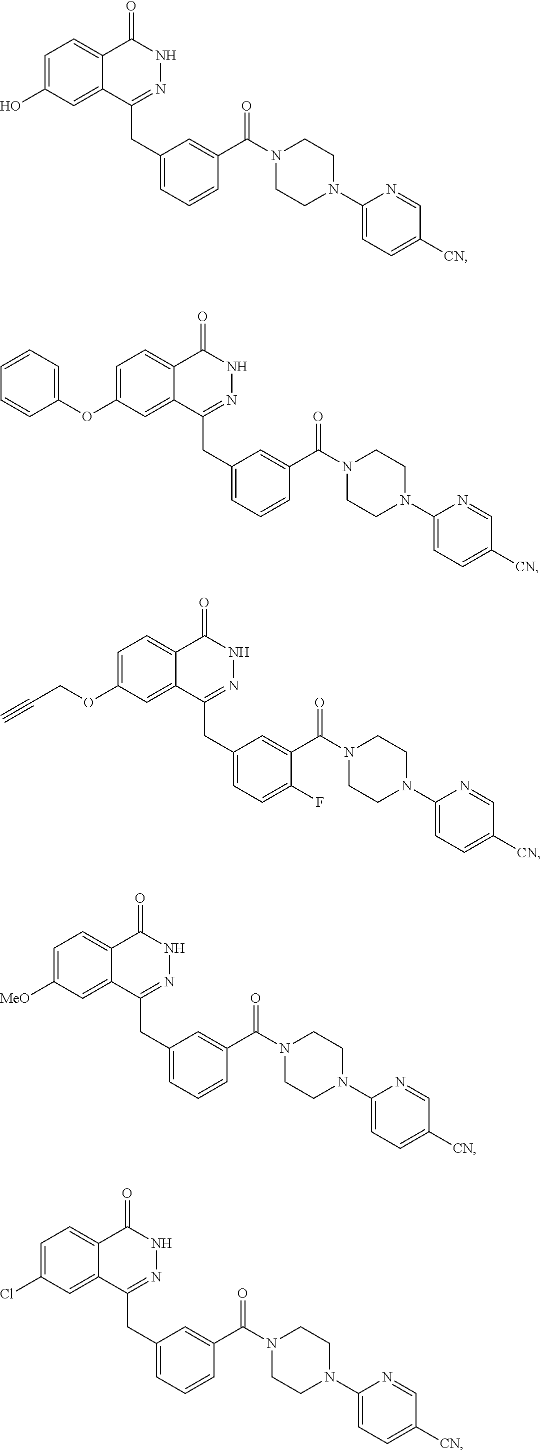 [SMILES: N#Cc1ccc(N2CCN(C(=O)c3cccc(Cc4n[nH]c(=O)c5ccc(O)cc45)c3)CC2)nc1 ; N#Cc1ccc(N2CCN(C(=O)c3cccc(Cc4n[nH]c(=O)c5ccc(Oc6ccccc6)cc45)c3)CC2)nc1 ; C#CCOc1ccc2c(=O)[nH]nc(Cc3ccc(F)c(C(=O)N4CCN(c5ccc(C#N)cn5)CC4)c3)c2c1 ; COc1ccc2c(=O)[nH]nc(Cc3cccc(C(=O)N4CCN(c5ccc(C#N)cn5)CC4)c3)c2c1 ; N#Cc1ccc(N2CCN(C(=O)c3cccc(Cc4n[nH]c(=O)c5ccc(Cl)cc45)c3)CC2)nc1]

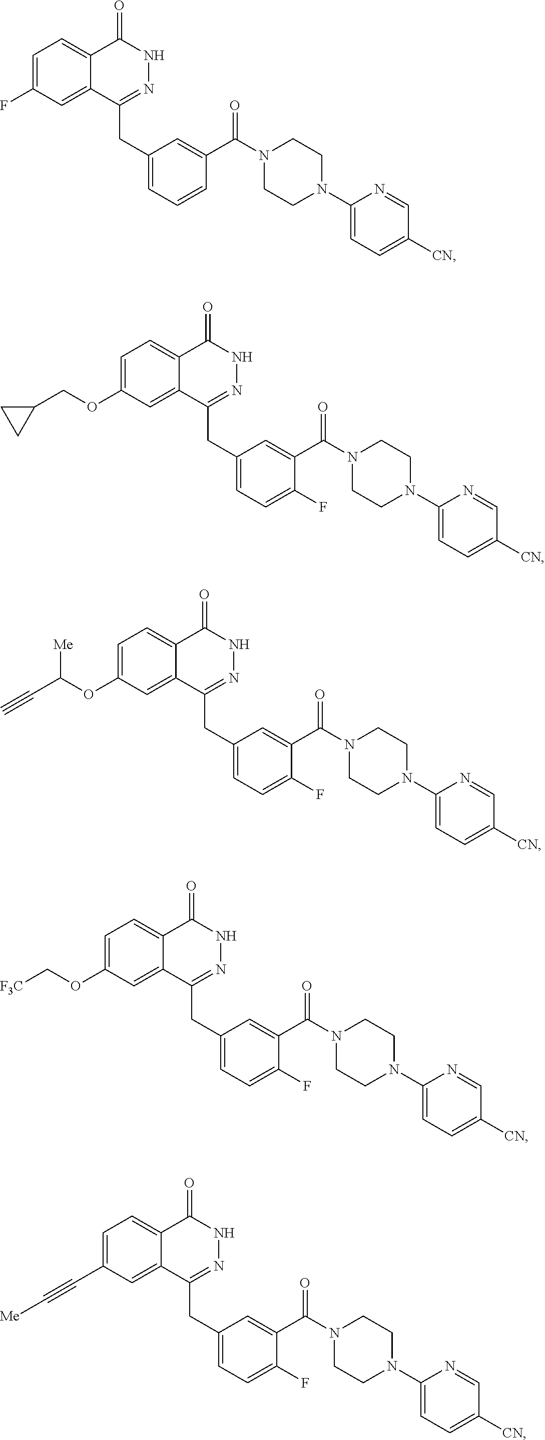 [SMILES: N#Cc1ccc(N2CCN(C(=O)c3cccc(Cc4n[nH]c(=O)c5ccc(F)cc45)c3)CC2)nc1 ; N#Cc1ccc(N2CCN(C(=O)c3cc(Cc4n[nH]c(=O)c5ccc(OCC6CC6)cc45)ccc3F)CC2)nc1 ; C#CC(C)Oc1ccc2c(=O)[nH]nc(Cc3ccc(F)c(C(=O)N4CCN(c5ccc(C#N)cn5)CC4)c3)c2c1 ; N#Cc1ccc(N2CCN(C(=O)c3cc(Cc4n[nH]c(=O)c5ccc(OCC(F)(F)F)cc45)ccc3F)CC2)nc1 ; CC#Cc1ccc2c(=O)[nH]nc(Cc3ccc(F)c(C(=O)N4CCN(c5ccc(C#N)cn5)CC4)c3)c2c1]

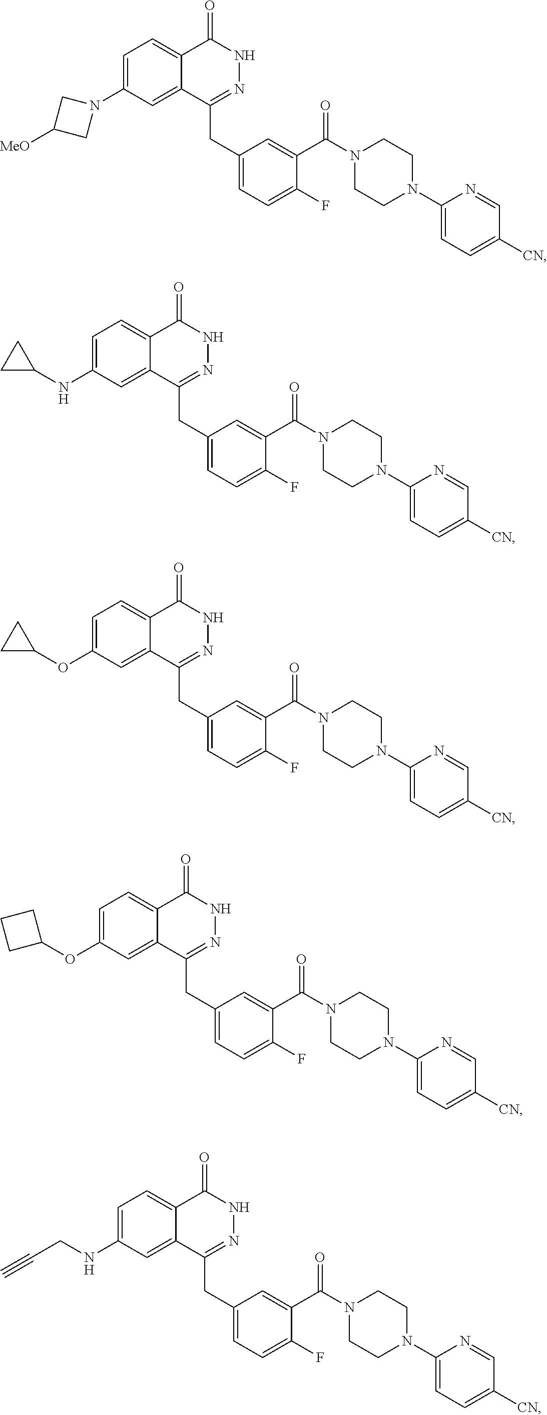 [SMILES: COC1CN(c2ccc3c(=O)[nH]nc(Cc4ccc(F)c(C(=O)N5CCN(c6ccc(C#N)cn6)CC5)c4)c3c2)C1 ; N#Cc1ccc(N2CCN(C(=O)c3cc(Cc4n[nH]c(=O)c5ccc(NC6CC6)cc45)ccc3F)CC2)nc1 ; N#Cc1ccc(N2CCN(C(=O)c3cc(Cc4n[nH]c(=O)c5ccc(OC6CC6)cc45)ccc3F)CC2)nc1 ; N#Cc1ccc(N2CCN(C(=O)c3cc(Cc4n[nH]c(=O)c5ccc(OC6CCC6)cc45)ccc3F)CC2)nc1 ; C#CCNc1ccc2c(=O)[nH]nc(Cc3ccc(F)c(C(=O)N4CCN(c5ccc(C#N)cn5)CC4)c3)c2c1]

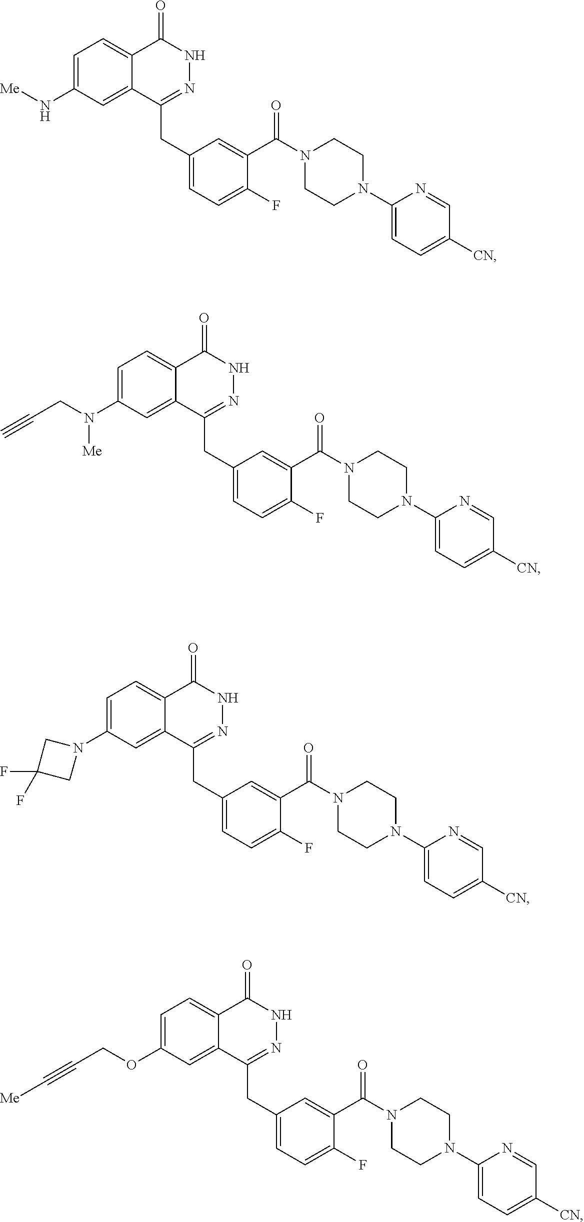 [SMILES: CNc1ccc2c(=O)[nH]nc(Cc3ccc(F)c(C(=O)N4CCN(c5ccc(C#N)cn5)CC4)c3)c2c1 ; C#CCN(C)c1ccc2c(=O)[nH]nc(Cc3ccc(F)c(C(=O)N4CCN(c5ccc(C#N)cn5)CC4)c3)c2c1 ; N#Cc1ccc(N2CCN(C(=O)c3cc(Cc4n[nH]c(=O)c5ccc(N6CC(F)(F)C6)cc45)ccc3F)CC2)nc1 ; CC#CCOc1ccc2c(=O)[nH]nc(Cc3ccc(F)c(C(=O)N4CCN(c5ccc(C#N)cn5)CC4)c3)c2c1]

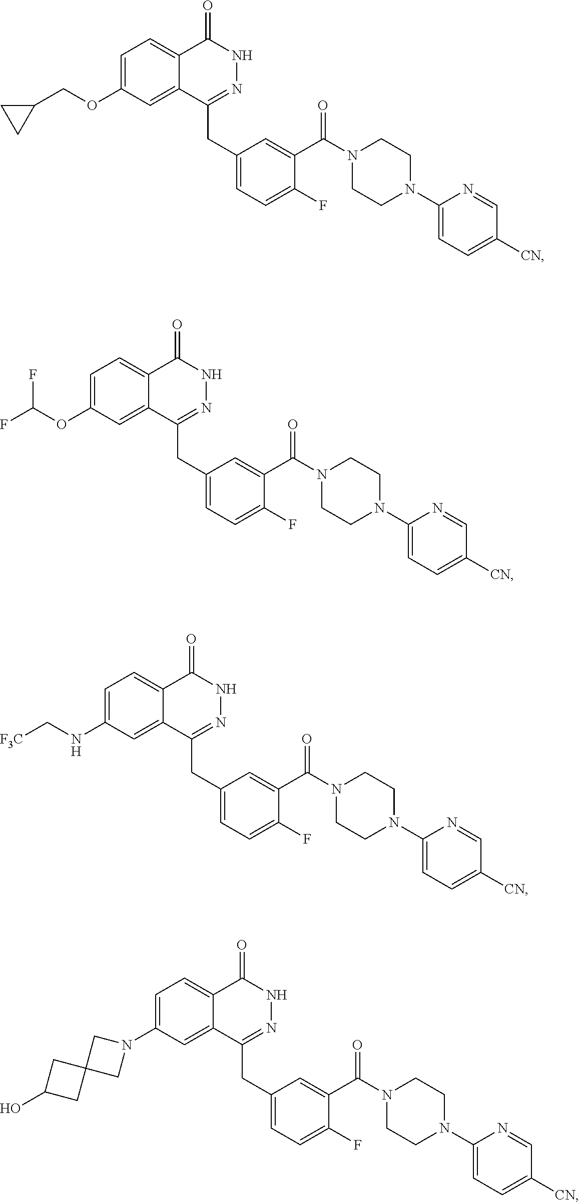 [SMILES: N#Cc1ccc(N2CCN(C(=O)c3cc(Cc4n[nH]c(=O)c5ccc(OCC6CC6)cc45)ccc3F)CC2)nc1 ; N#Cc1ccc(N2CCN(C(=O)c3cc(Cc4n[nH]c(=O)c5ccc(OC(F)F)cc45)ccc3F)CC2)nc1 ; N#Cc1ccc(N2CCN(C(=O)c3cc(Cc4n[nH]c(=O)c5ccc(NCC(F)(F)F)cc45)ccc3F)CC2)nc1 ; N#Cc1ccc(N2CCN(C(=O)c3cc(Cc4n[nH]c(=O)c5ccc(N6CC7(CC(O)C7)C6)cc45)ccc3F)CC2)nc1]

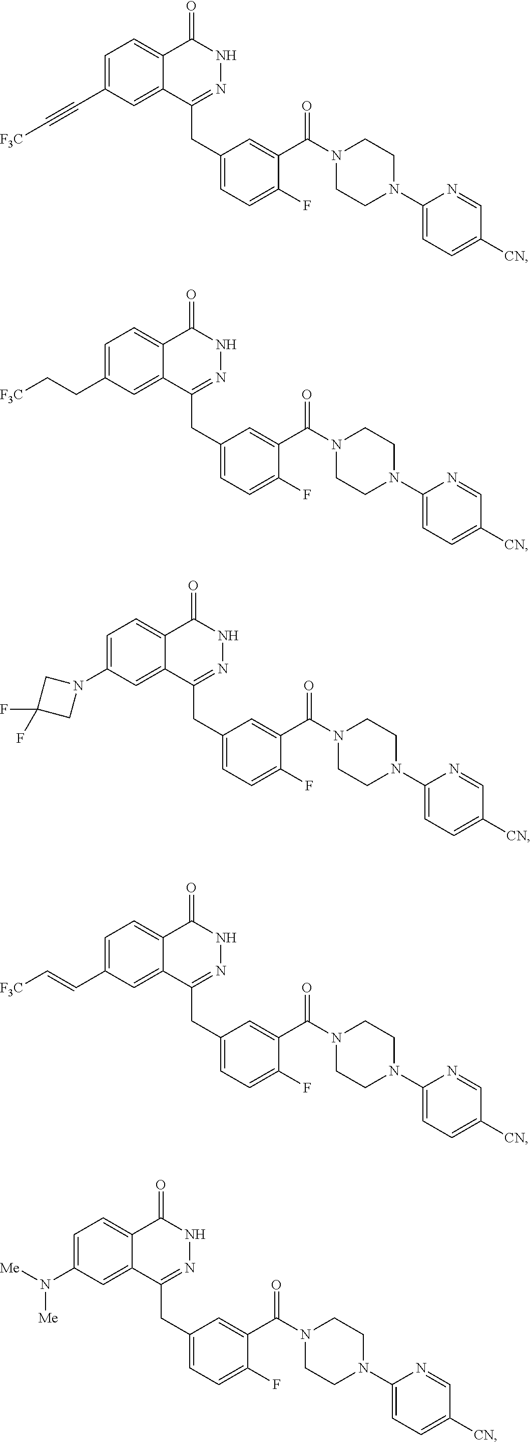 [SMILES: N#Cc1ccc(N2CCN(C(=O)c3cc(Cc4n[nH]c(=O)c5ccc(C#CC(F)(F)F)cc45)ccc3F)CC2)nc1 ; N#Cc1ccc(N2CCN(C(=O)c3cc(Cc4n[nH]c(=O)c5ccc(CCC(F)(F)F)cc45)ccc3F)CC2)nc1 ; N#Cc1ccc(N2CCN(C(=O)c3cc(Cc4n[nH]c(=O)c5ccc(N6CC(F)(F)C6)cc45)ccc3F)CC2)nc1 ; N#Cc1ccc(N2CCN(C(=O)c3cc(Cc4n[nH]c(=O)c5ccc(/C=C/C(F)(F)F)cc45)ccc3F)CC2)nc1 ; CN(C)c1ccc2c(=O)[nH]nc(Cc3ccc(F)c(C(=O)N4CCN(c5ccc(C#N)cn5)CC4)c3)c2c1]

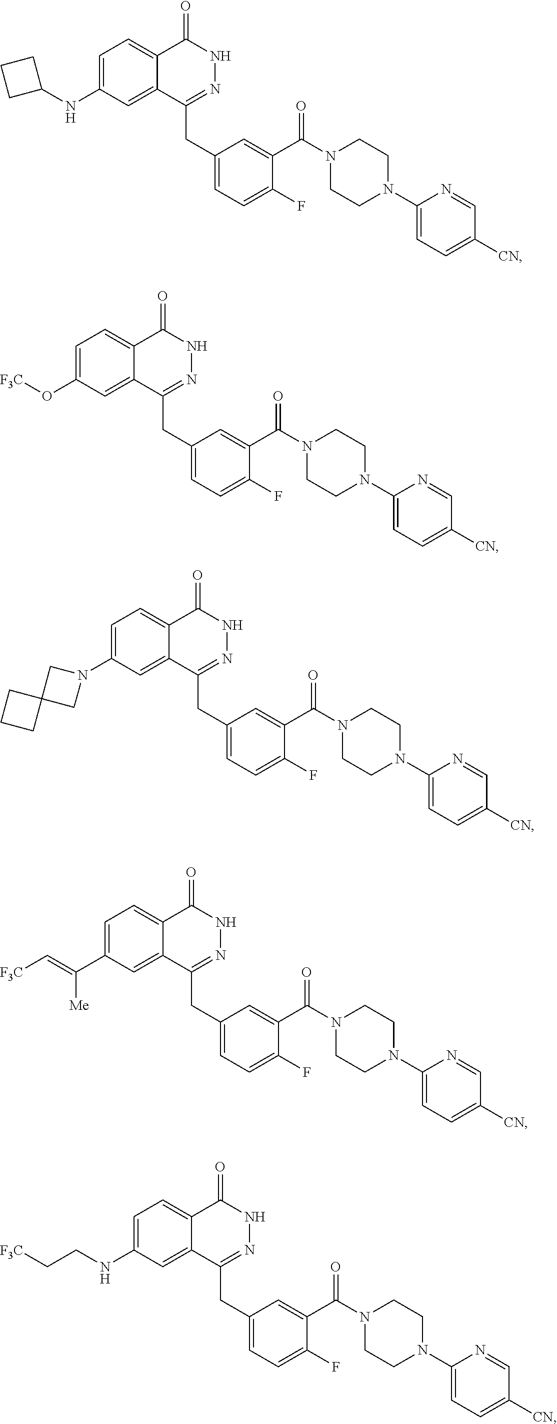 [SMILES: N#Cc1ccc(N2CCN(C(=O)c3cc(Cc4n[nH]c(=O)c5ccc(NC6CCC6)cc45)ccc3F)CC2)nc1 ; N#Cc1ccc(N2CCN(C(=O)c3cc(Cc4n[nH]c(=O)c5ccc(OC(F)(F)F)cc45)ccc3F)CC2)nc1 ; N#Cc1ccc(N2CCN(C(=O)c3cc(Cc4n[nH]c(=O)c5ccc(N6CC7(CCC7)C6)cc45)ccc3F)CC2)nc1 ; C/C(=C\C(F)(F)F)c1ccc2c(=O)[nH]nc(Cc3ccc(F)c(C(=O)N4CCN(c5ccc(C#N)cn5)CC4)c3)c2c1 ; N#Cc1ccc(N2CCN(C(=O)c3cc(Cc4n[nH]c(=O)c5ccc(NCCC(F)(F)F)cc45)ccc3F)CC2)nc1]

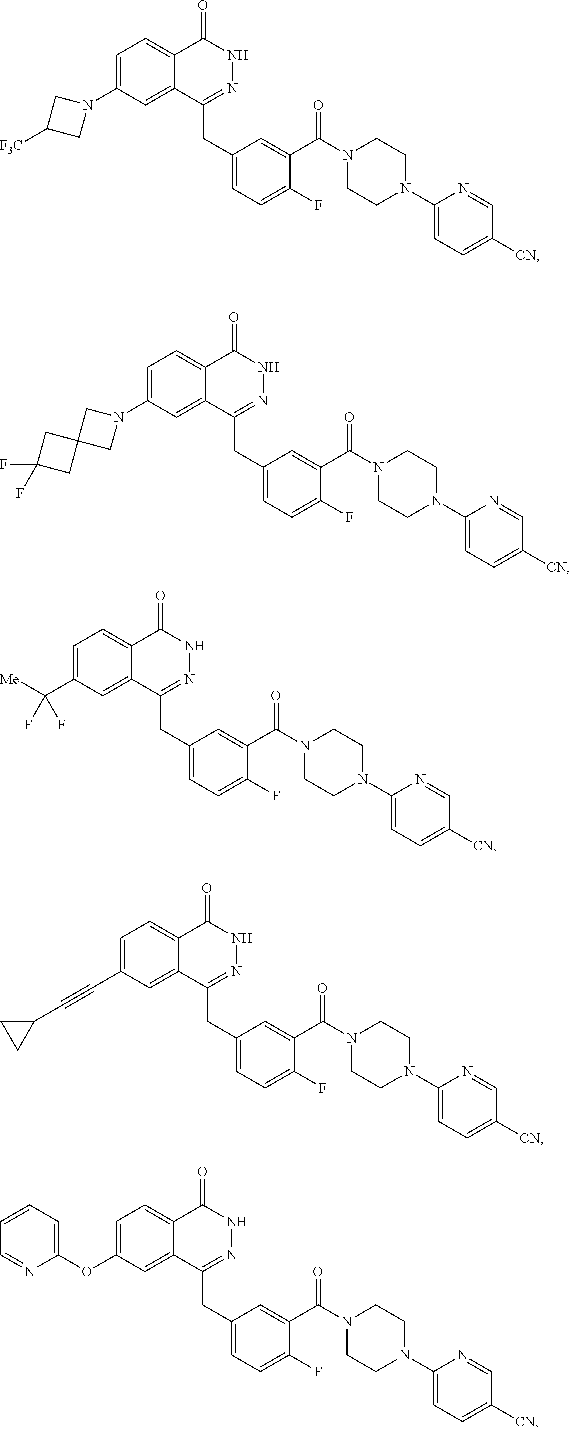 [SMILES: N#Cc1ccc(N2CCN(C(=O)c3cc(Cc4n[nH]c(=O)c5ccc(N6CC(C(F)(F)F)C6)cc45)ccc3F)CC2)nc1 ; N#Cc1ccc(N2CCN(C(=O)c3cc(Cc4n[nH]c(=O)c5ccc(N6CC7(C6)CC(F)(F)C7)cc45)ccc3F)CC2)nc1 ; CC(F)(F)c1ccc2c(=O)[nH]nc(Cc3ccc(F)c(C(=O)N4CCN(c5ccc(C#N)cn5)CC4)c3)c2c1 ; N#Cc1ccc(N2CCN(C(=O)c3cc(Cc4n[nH]c(=O)c5ccc(C#CC6CC6)cc45)ccc3F)CC2)nc1 ; N#Cc1ccc(N2CCN(C(=O)c3cc(Cc4n[nH]c(=O)c5ccc(Oc6ccccn6)cc45)ccc3F)CC2)nc1]

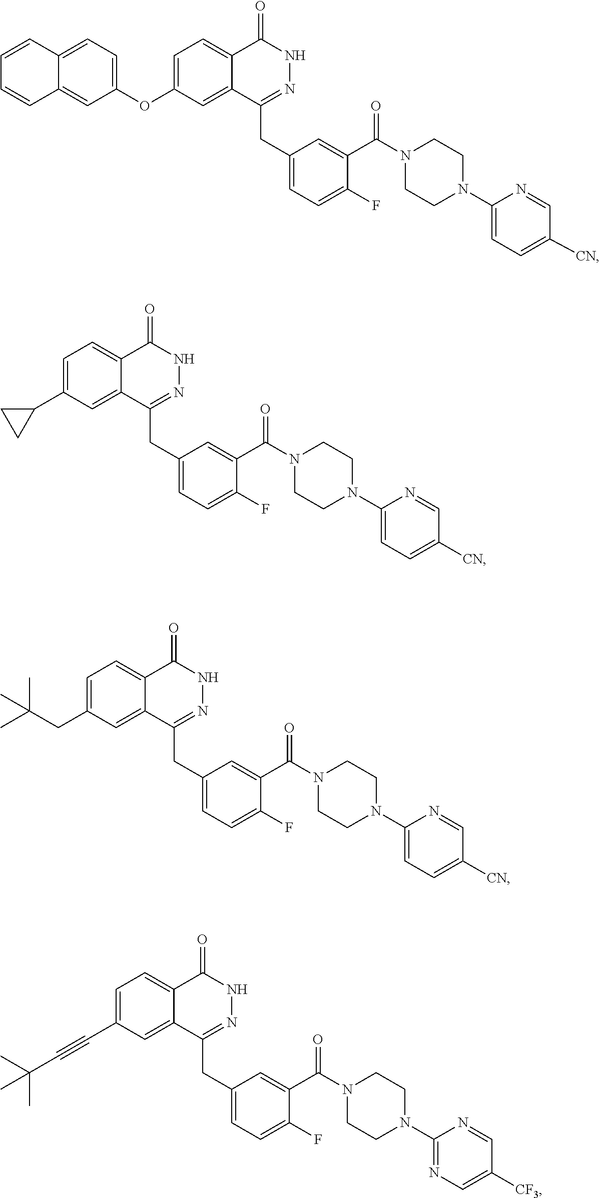 [SMILES: N#Cc1ccc(N2CCN(C(=O)c3cc(Cc4n[nH]c(=O)c5ccc(Oc6ccc7ccccc7c6)cc45)ccc3F)CC2)nc1 ; N#Cc1ccc(N2CCN(C(=O)c3cc(Cc4n[nH]c(=O)c5ccc(C6CC6)cc45)ccc3F)CC2)nc1 ; CC(C)(C)Cc1ccc2c(=O)[nH]nc(Cc3ccc(F)c(C(=O)N4CCN(c5ccc(C#N)cn5)CC4)c3)c2c1 ; CC(C)(C)C#Cc1ccc2c(=O)[nH]nc(Cc3ccc(F)c(C(=O)N4CCN(c5ncc(C(F)(F)F)cn5)CC4)c3)c2c1]

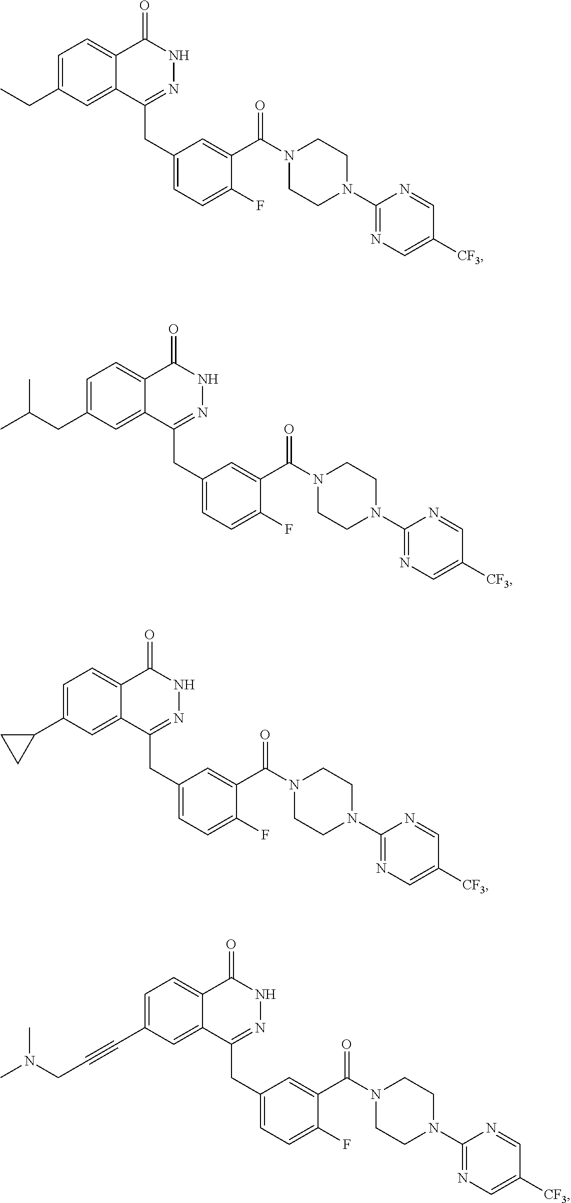 [SMILES: CCc1ccc2c(=O)[nH]nc(Cc3ccc(F)c(C(=O)N4CCN(c5ncc(C(F)(F)F)cn5)CC4)c3)c2c1 ; CC(C)Cc1ccc2c(=O)[nH]nc(Cc3ccc(F)c(C(=O)N4CCN(c5ncc(C(F)(F)F)cn5)CC4)c3)c2c1 ; O=C(c1cc(Cc2n[nH]c(=O)c3ccc(C4CC4)cc23)ccc1F)N1CCN(c2ncc(C(F)(F)F)cn2)CC1 ; CN(C)CC#Cc1ccc2c(=O)[nH]nc(Cc3ccc(F)c(C(=O)N4CCN(c5ncc(C(F)(F)F)cn5)CC4)c3)c2c1]

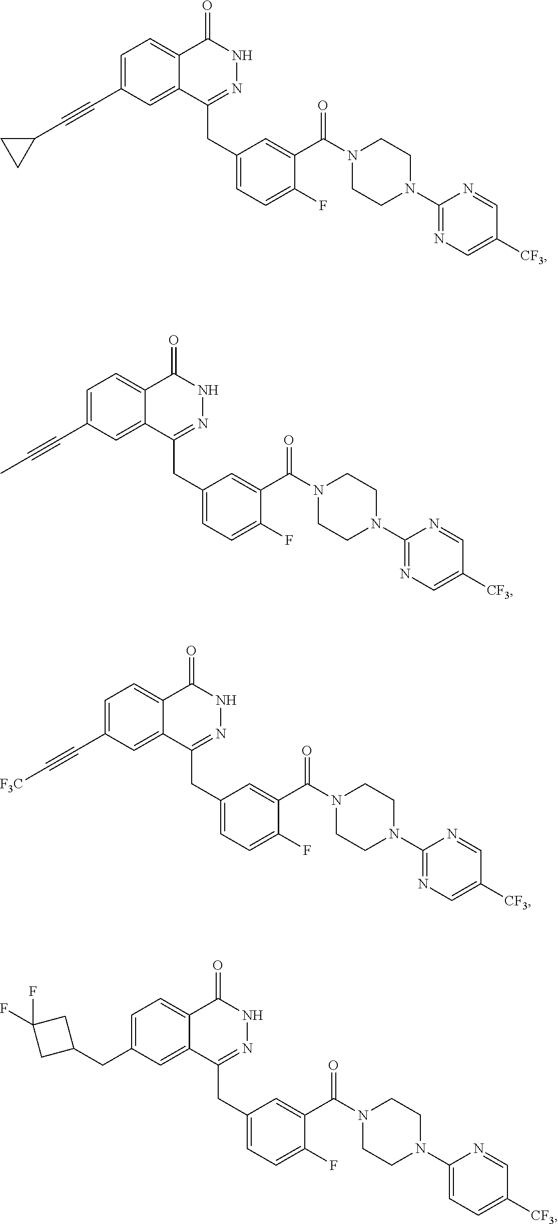 [SMILES: O=C(c1cc(Cc2n[nH]c(=O)c3ccc(C#CC4CC4)cc23)ccc1F)N1CCN(c2ncc(C(F)(F)F)cn2)CC1 ; CC#Cc1ccc2c(=O)[nH]nc(Cc3ccc(F)c(C(=O)N4CCN(c5ncc(C(F)(F)F)cn5)CC4)c3)c2c1 ; O=C(c1cc(Cc2n[nH]c(=O)c3ccc(C#CC(F)(F)F)cc23)ccc1F)N1CCN(c2ncc(C(F)(F)F)cn2)CC1 ; O=C(c1cc(Cc2n[nH]c(=O)c3ccc(CC4CC(F)(F)C4)cc23)ccc1F)N1CCN(c2ccc(C(F)(F)F)cn2)CC1]

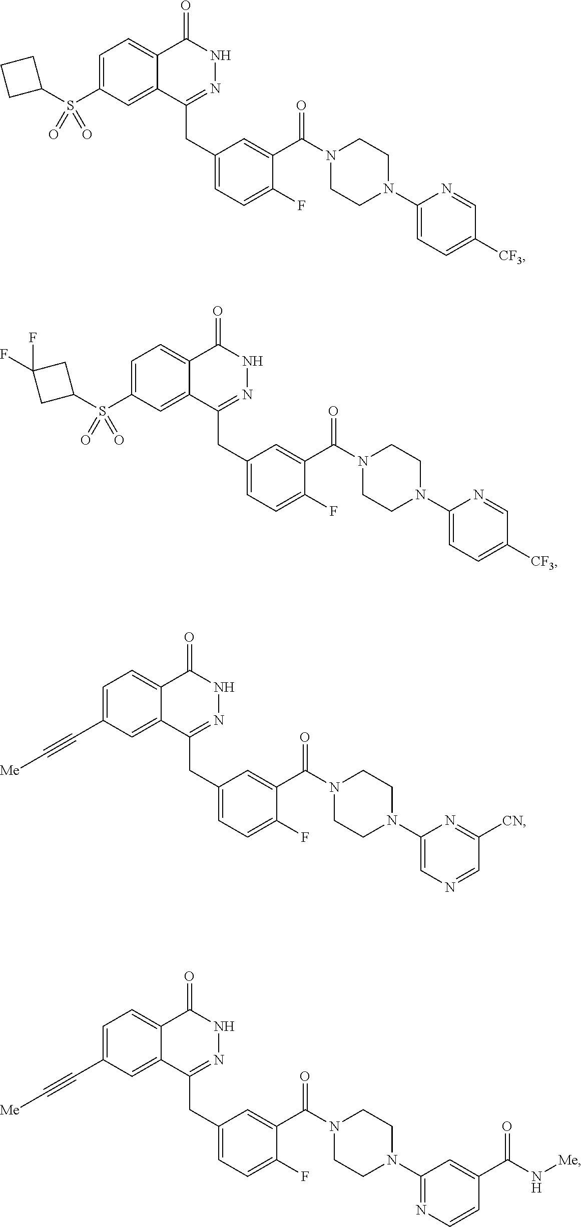 [SMILES: O=C(c1cc(Cc2n[nH]c(=O)c3ccc(S(=O)(=O)C4CCC4)cc23)ccc1F)N1CCN(c2ccc(C(F)(F)F)cn2)CC1 ; O=C(c1cc(Cc2n[nH]c(=O)c3ccc(S(=O)(=O)C4CC(F)(F)C4)cc23)ccc1F)N1CCN(c2ccc(C(F)(F)F)cn2)CC1 ; CC#Cc1ccc2c(=O)[nH]nc(Cc3ccc(F)c(C(=O)N4CCN(c5cncc(C#N)n5)CC4)c3)c2c1 ; CC#Cc1ccc2c(=O)[nH]nc(Cc3ccc(F)c(C(=O)N4CCN(c5cc(C(=O)NC)ccn5)CC4)c3)c2c1]

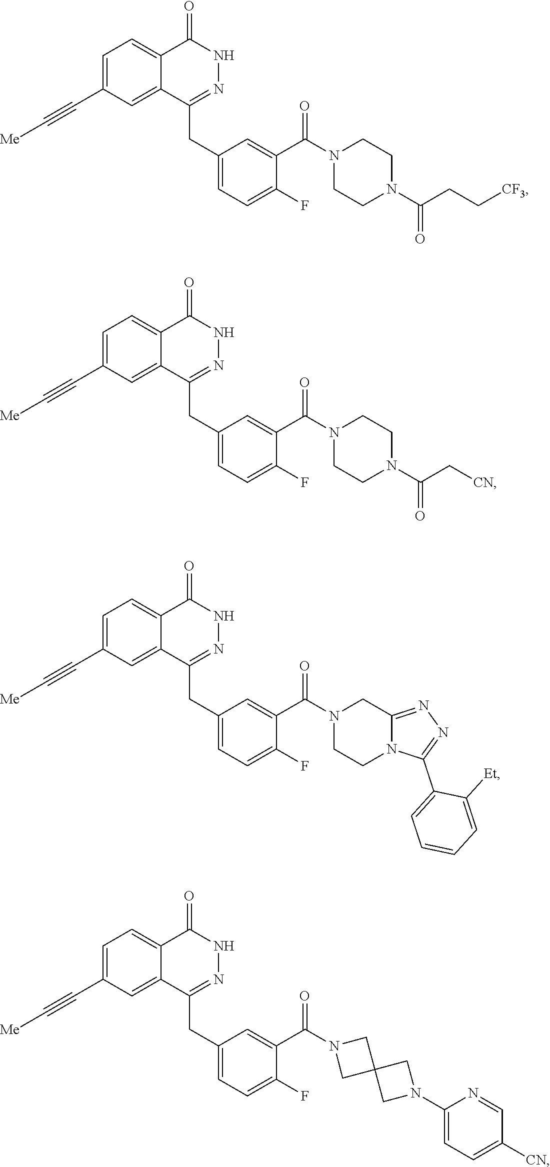 [SMILES: CC#Cc1ccc2c(=O)[nH]nc(Cc3ccc(F)c(C(=O)N4CCN(C(=O)CCC(F)(F)F)CC4)c3)c2c1 ; CC#Cc1ccc2c(=O)[nH]nc(Cc3ccc(F)c(C(=O)N4CCN(C(=O)CC#N)CC4)c3)c2c1 ; CC#Cc1ccc2c(=O)[nH]nc(Cc3ccc(F)c(C(=O)N4CCn5c(nnc5-c5ccccc5CC)C4)c3)c2c1 ; CC#Cc1ccc2c(=O)[nH]nc(Cc3ccc(F)c(C(=O)N4CC5(C4)CN(c4ccc(C#N)cn4)C5)c3)c2c1]

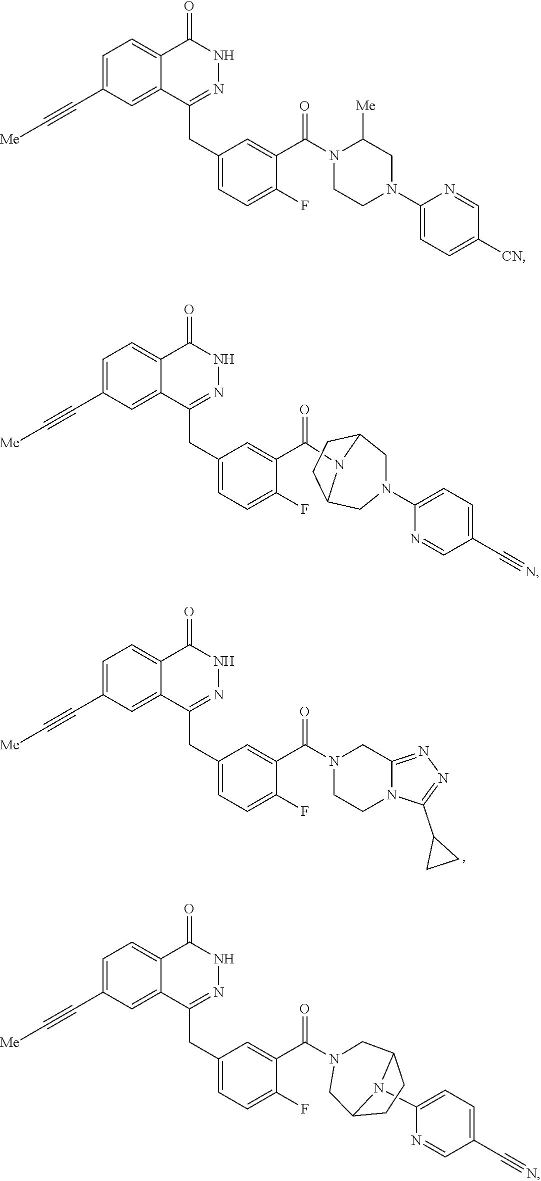 [SMILES: CC#Cc1ccc2c(=O)[nH]nc(Cc3ccc(F)c(C(=O)N4CCN(c5ccc(C#N)cn5)CC4C)c3)c2c1 ; CC#Cc1ccc2c(=O)[nH]nc(Cc3ccc(F)c(C(=O)N4C5CCC4CN(c4ccc(C#N)cn4)C5)c3)c2c1 ; CC#Cc1ccc2c(=O)[nH]nc(Cc3ccc(F)c(C(=O)N4CCn5c(nnc5C5CC5)C4)c3)c2c1 ; CC#Cc1ccc2c(=O)[nH]nc(Cc3ccc(F)c(C(=O)N4CC5CCC(C4)N5c4ccc(C#N)cn4)c3)c2c1]

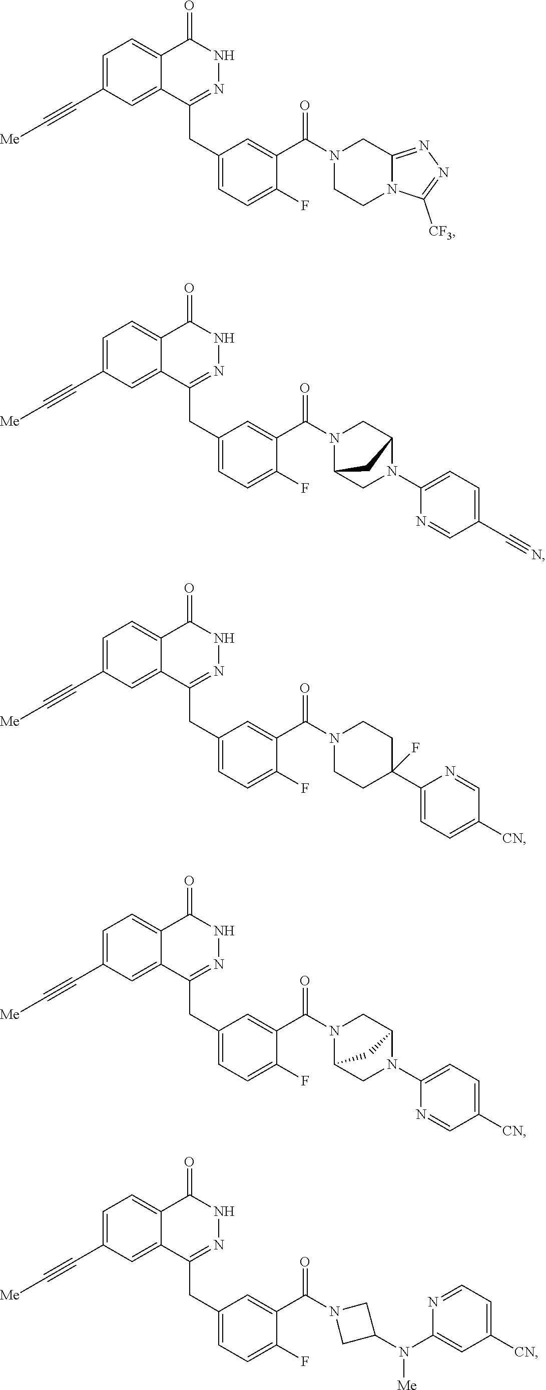 [SMILES: CC#Cc1ccc2c(=O)[nH]nc(Cc3ccc(F)c(C(=O)N4CCn5c(nnc5C(F)(F)F)C4)c3)c2c1 ; CC#Cc1ccc2c(=O)[nH]nc(Cc3ccc(F)c(C(=O)N4C[C@H]5C[C@@H]4CN5c4ccc(C#N)cn4)c3)c2c1 ; CC#Cc1ccc2c(=O)[nH]nc(Cc3ccc(F)c(C(=O)N4CCC(F)(c5ccc(C#N)cn5)CC4)c3)c2c1 ; CC#Cc1ccc2c(=O)[nH]nc(Cc3ccc(F)c(C(=O)N4C[C@@H]5C[C@H]4CN5c4ccc(C#N)cn4)c3)c2c1 ; CC#Cc1ccc2c(=O)[nH]nc(Cc3ccc(F)c(C(=O)N4CC(N(C)c5cc(C#N)ccn5)C4)c3)c2c1]

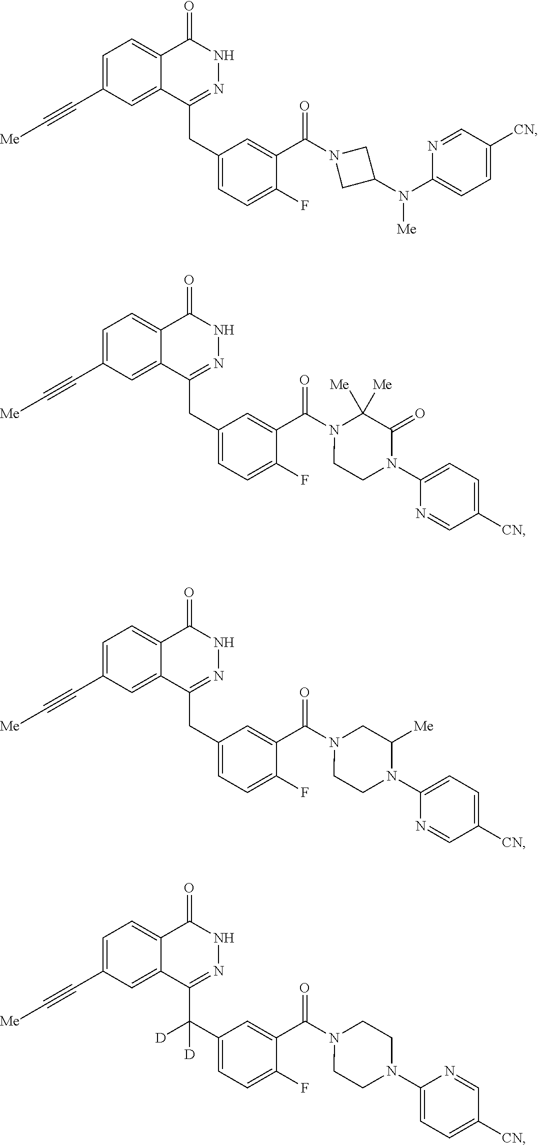 [SMILES: CC#Cc1ccc2c(=O)[nH]nc(Cc3ccc(F)c(C(=O)N4CC(N(C)c5ccc(C#N)cn5)C4)c3)c2c1 ; CC#Cc1ccc2c(=O)[nH]nc(Cc3ccc(F)c(C(=O)N4CCN(c5ccc(C#N)cn5)C(=O)C4(C)C)c3)c2c1 ; CC#Cc1ccc2c(=O)[nH]nc(Cc3ccc(F)c(C(=O)N4CCN(c5ccc(C#N)cn5)C(C)C4)c3)c2c1 ; [2H]C([2H])(c1ccc(F)c(C(=O)N2CCN(c3ccc(C#N)cn3)CC2)c1)c1n[nH]c(=O)c2ccc(C#CC)cc12]

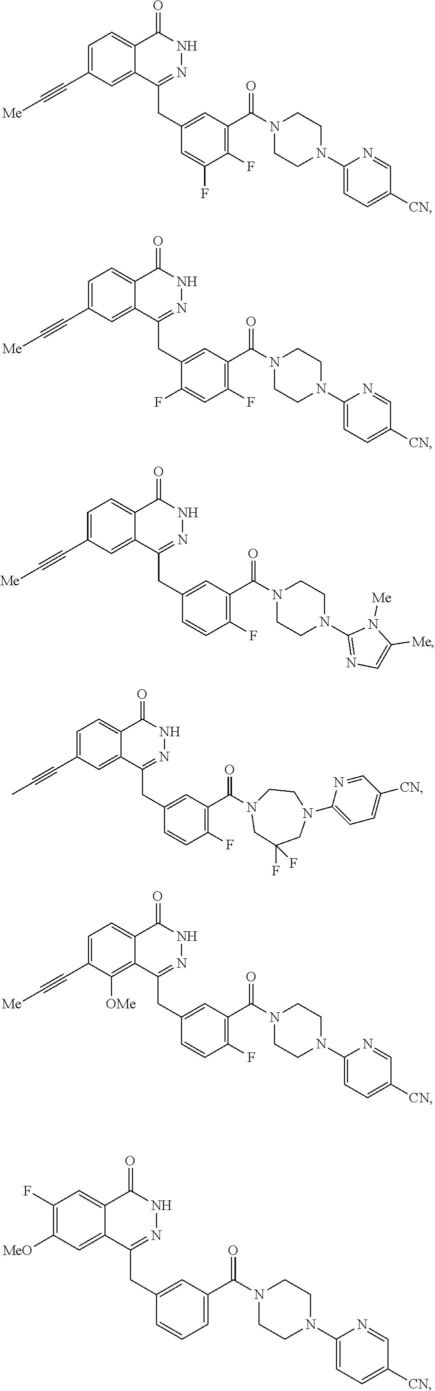 [SMILES: CC#Cc1ccc2c(=O)[nH]nc(Cc3cc(F)c(F)c(C(=O)N4CCN(c5ccc(C#N)cn5)CC4)c3)c2c1 ; CC#Cc1ccc2c(=O)[nH]nc(Cc3cc(C(=O)N4CCN(c5ccc(C#N)cn5)CC4)c(F)cc3F)c2c1 ; CC#Cc1ccc2c(=O)[nH]nc(Cc3ccc(F)c(C(=O)N4CCN(c5ncc(C)n5C)CC4)c3)c2c1 ; CC#Cc1ccc2c(=O)[nH]nc(Cc3ccc(F)c(C(=O)N4CCN(c5ccc(C#N)cn5)CC(F)(F)C4)c3)c2c1 ; CC#Cc1ccc2c(=O)[nH]nc(Cc3ccc(F)c(C(=O)N4CCN(c5ccc(C#N)cn5)CC4)c3)c2c1OC ; COc1cc2c(Cc3cccc(C(=O)N4CCN(c5ccc(C#N)cn5)CC4)c3)n[nH]c(=O)c2cc1F]

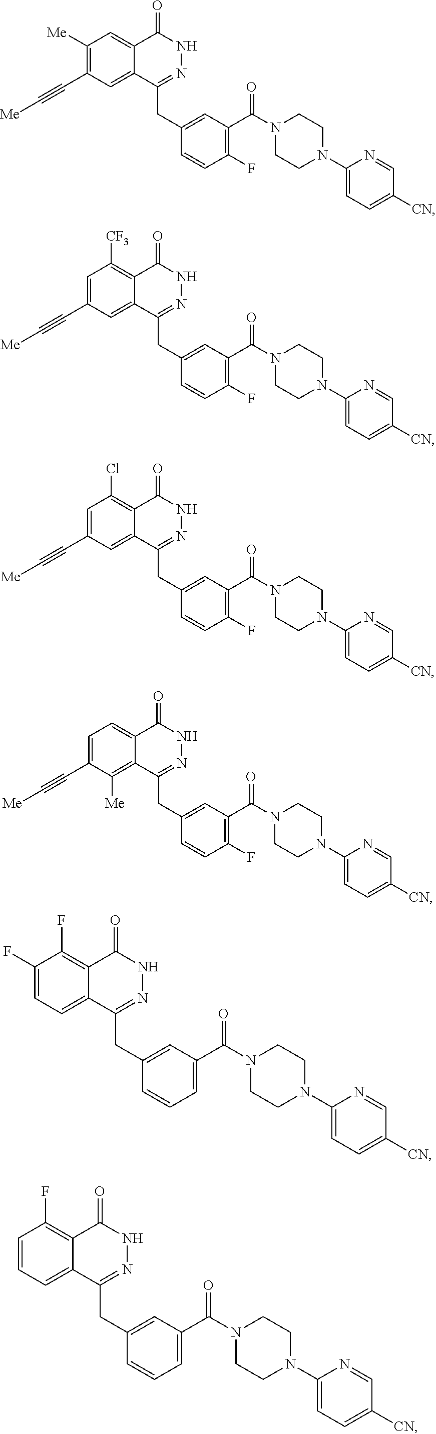 [SMILES: CC#Cc1cc2c(Cc3ccc(F)c(C(=O)N4CCN(c5ccc(C#N)cn5)CC4)c3)n[nH]c(=O)c2cc1C ; CC#Cc1cc(C(F)(F)F)c2c(=O)[nH]nc(Cc3ccc(F)c(C(=O)N4CCN(c5ccc(C#N)cn5)CC4)c3)c2c1 ; CC#Cc1cc(Cl)c2c(=O)[nH]nc(Cc3ccc(F)c(C(=O)N4CCN(c5ccc(C#N)cn5)CC4)c3)c2c1 ; CC#Cc1ccc2c(=O)[nH]nc(Cc3ccc(F)c(C(=O)N4CCN(c5ccc(C#N)cn5)CC4)c3)c2c1C ; N#Cc1ccc(N2CCN(C(=O)c3cccc(Cc4n[nH]c(=O)c5c(F)c(F)ccc45)c3)CC2)nc1 ; N#Cc1ccc(N2CCN(C(=O)c3cccc(Cc4n[nH]c(=O)c5c(F)cccc45)c3)CC2)nc1]

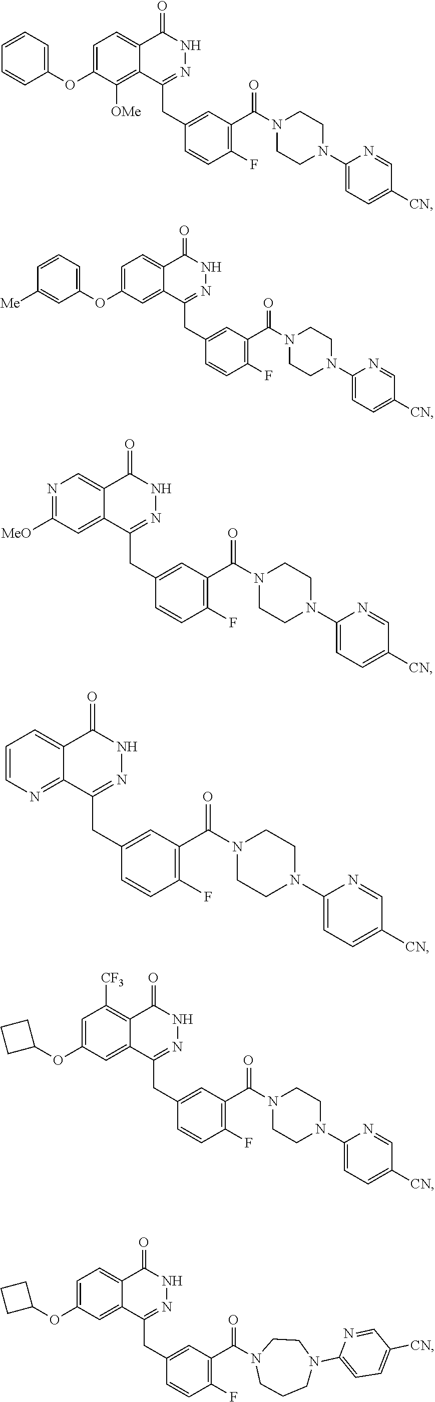 [SMILES: COc1c(Oc2ccccc2)ccc2c(=O)[nH]nc(Cc3ccc(F)c(C(=O)N4CCN(c5ccc(C#N)cn5)CC4)c3)c12 ; Cc1cccc(Oc2ccc3c(=O)[nH]nc(Cc4ccc(F)c(C(=O)N5CCN(c6ccc(C#N)cn6)CC5)c4)c3c2)c1 ; COc1cc2c(Cc3ccc(F)c(C(=O)N4CCN(c5ccc(C#N)cn5)CC4)c3)n[nH]c(=O)c2cn1 ; N#Cc1ccc(N2CCN(C(=O)c3cc(Cc4n[nH]c(=O)c5cccnc45)ccc3F)CC2)nc1 ; N#Cc1ccc(N2CCN(C(=O)c3cc(Cc4n[nH]c(=O)c5c(C(F)(F)F)cc(OC6CCC6)cc45)ccc3F)CC2)nc1 ; N#Cc1ccc(N2CCCN(C(=O)c3cc(Cc4n[nH]c(=O)c5ccc(OC6CCC6)cc45)ccc3F)CC2)nc1]

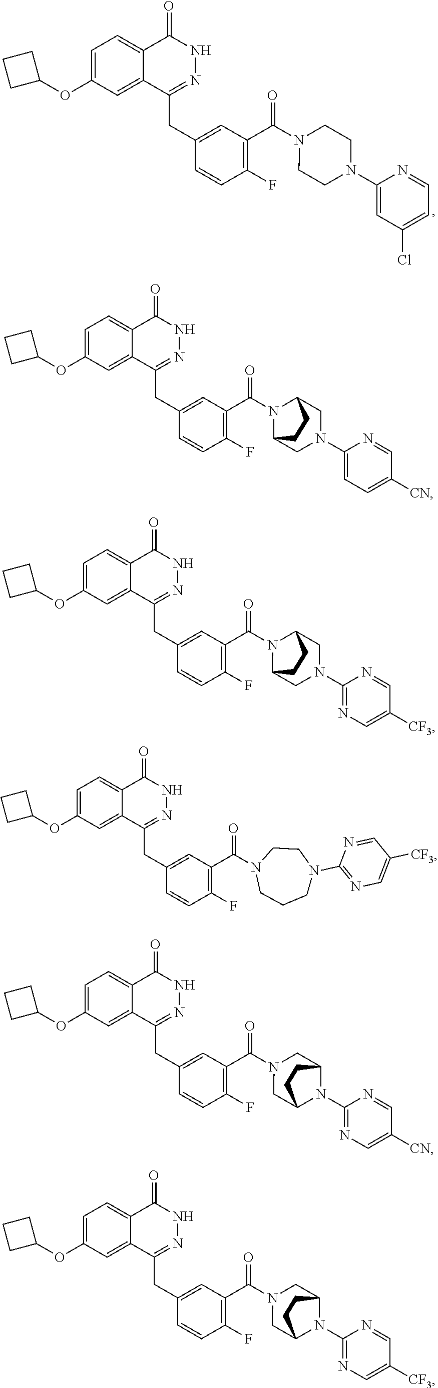

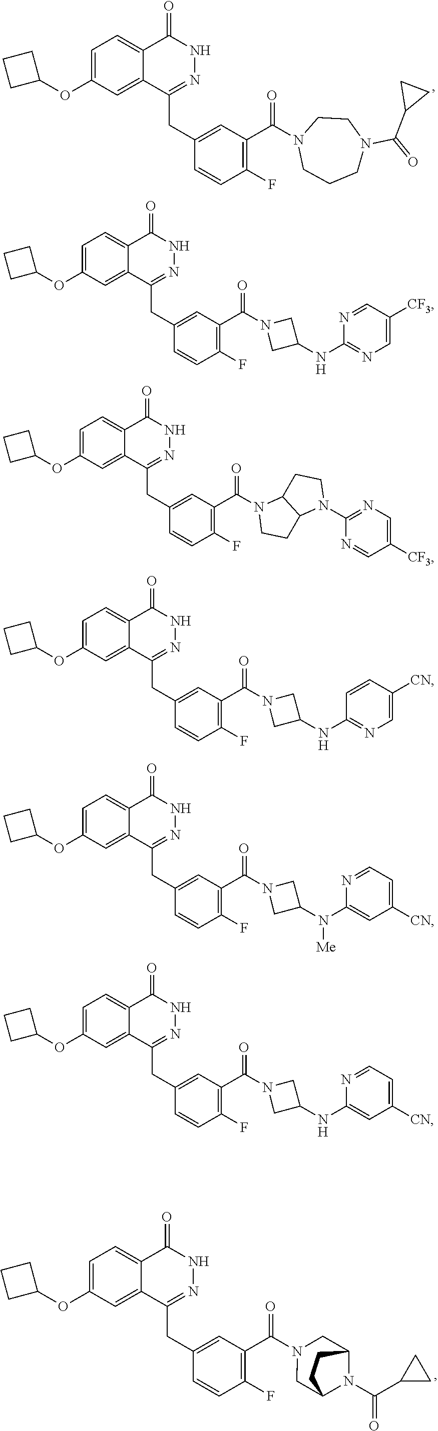

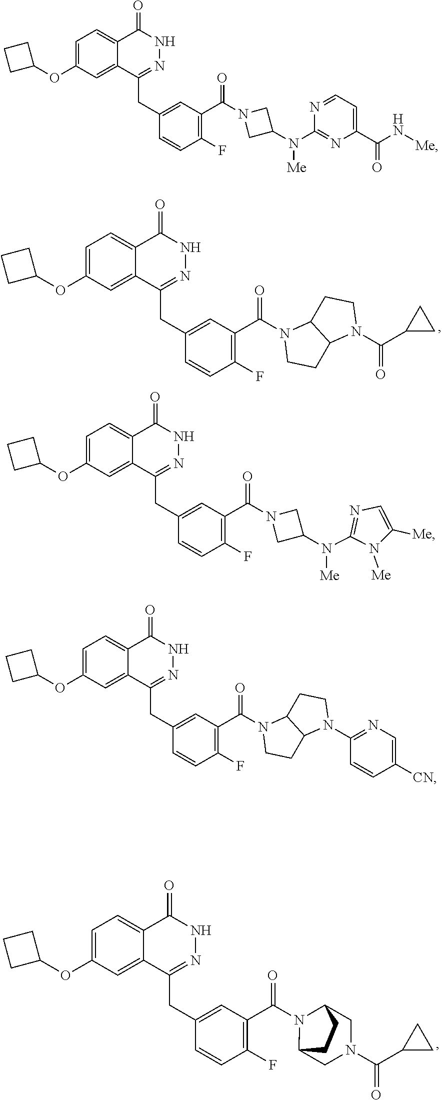

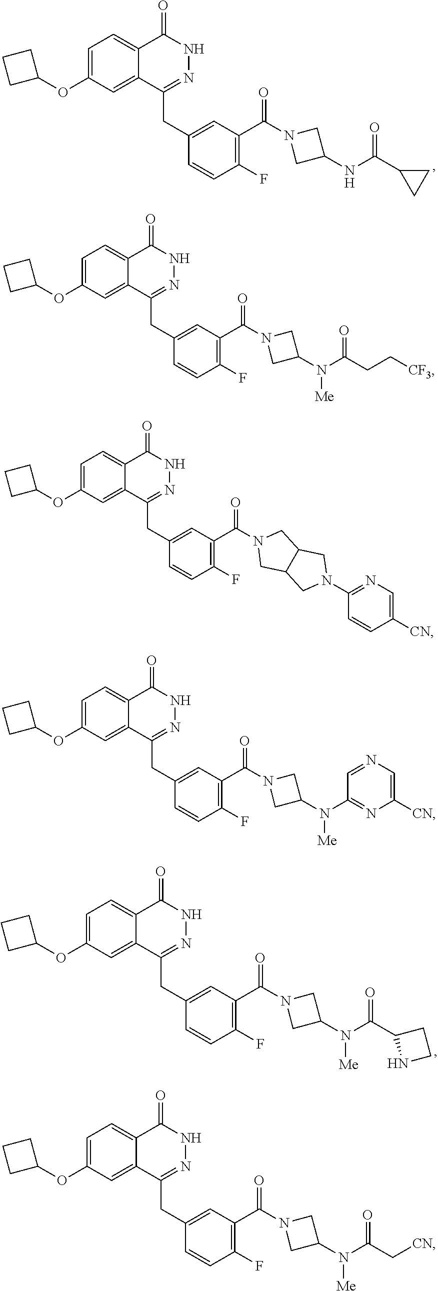

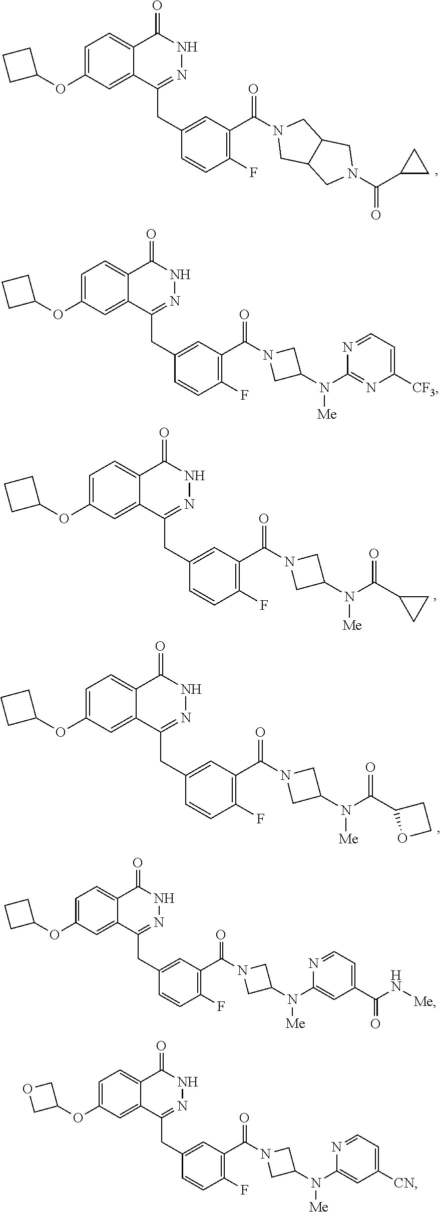

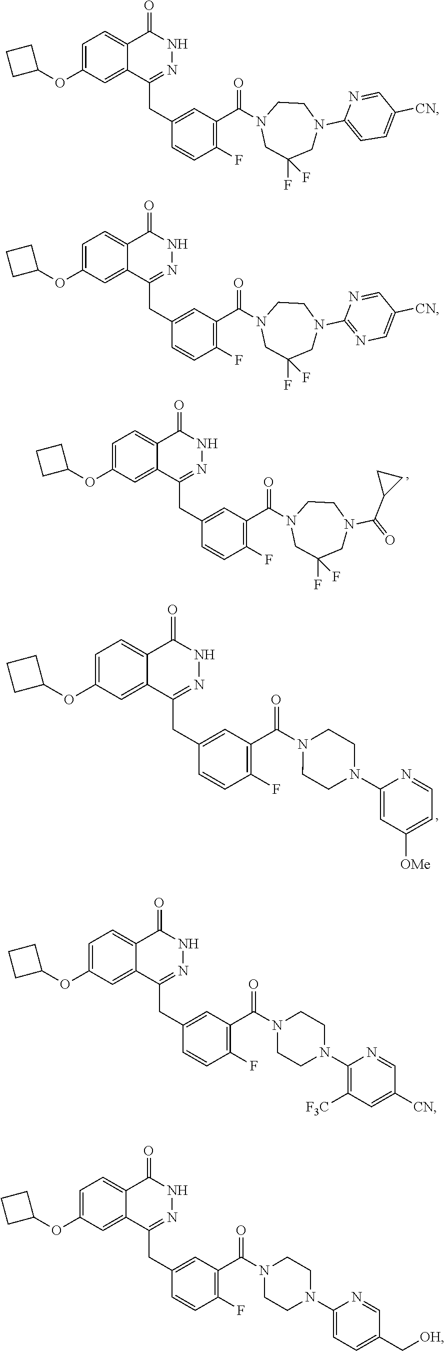 [SMILES: N#Cc1ccc(N2CCN(C(=O)c3cc(Cc4n[nH]c(=O)c5ccc(OC6CCC6)cc45)ccc3F)CC(F)(F)C2)nc1 ; N#Cc1cnc(N2CCN(C(=O)c3cc(Cc4n[nH]c(=O)c5ccc(OC6CCC6)cc45)ccc3F)CC(F)(F)C2)nc1 ; O=C(c1cc(Cc2n[nH]c(=O)c3ccc(OC4CCC4)cc23)ccc1F)N1CCN(C(=O)C2CC2)CC(F)(F)C1 ; COc1ccnc(N2CCN(C(=O)c3cc(Cc4n[nH]c(=O)c5ccc(OC6CCC6)cc45)ccc3F)CC2)c1 ; N#Cc1cnc(N2CCN(C(=O)c3cc(Cc4n[nH]c(=O)c5ccc(OC6CCC6)cc45)ccc3F)CC2)c(C(F)(F)F)c1 ; O=C(c1cc(Cc2n[nH]c(=O)c3ccc(OC4CCC4)cc23)ccc1F)N1CCN(c2ccc(CO)cn2)CC1]

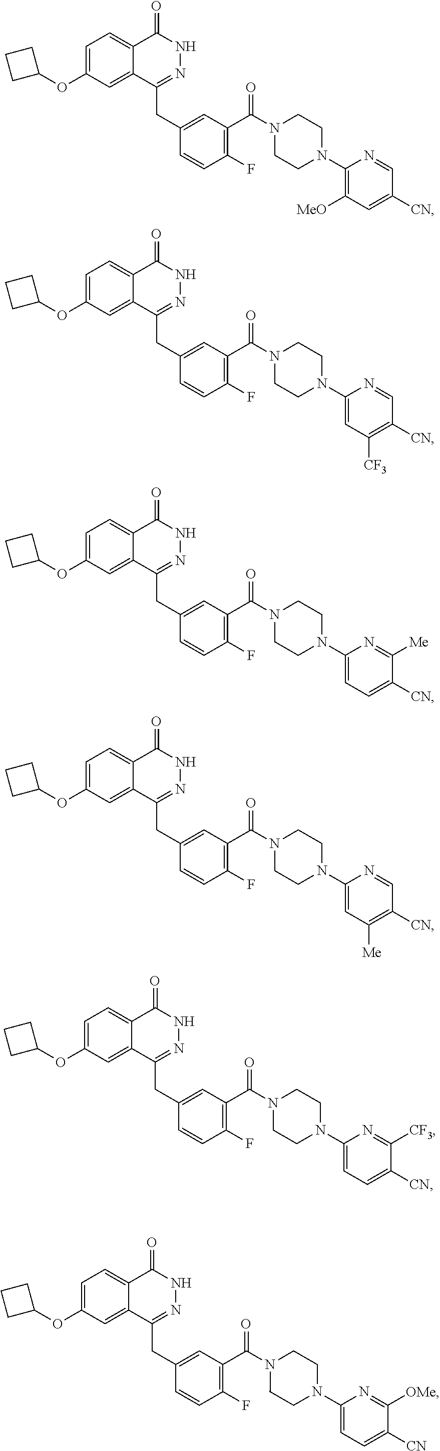 [SMILES: COc1cc(C#N)cnc1N1CCN(C(=O)c2cc(Cc3n[nH]c(=O)c4ccc(OC5CCC5)cc34)ccc2F)CC1 ; N#Cc1cnc(N2CCN(C(=O)c3cc(Cc4n[nH]c(=O)c5ccc(OC6CCC6)cc45)ccc3F)CC2)cc1C(F)(F)F ; Cc1nc(N2CCN(C(=O)c3cc(Cc4n[nH]c(=O)c5ccc(OC6CCC6)cc45)ccc3F)CC2)ccc1C#N ; Cc1cc(N2CCN(C(=O)c3cc(Cc4n[nH]c(=O)c5ccc(OC6CCC6)cc45)ccc3F)CC2)ncc1C#N ; N#Cc1ccc(N2CCN(C(=O)c3cc(Cc4n[nH]c(=O)c5ccc(OC6CCC6)cc45)ccc3F)CC2)nc1C(F)(F)F ; COc1nc(N2CCN(C(=O)c3cc(Cc4n[nH]c(=O)c5ccc(OC6CCC6)cc45)ccc3F)CC2)ccc1C#N]

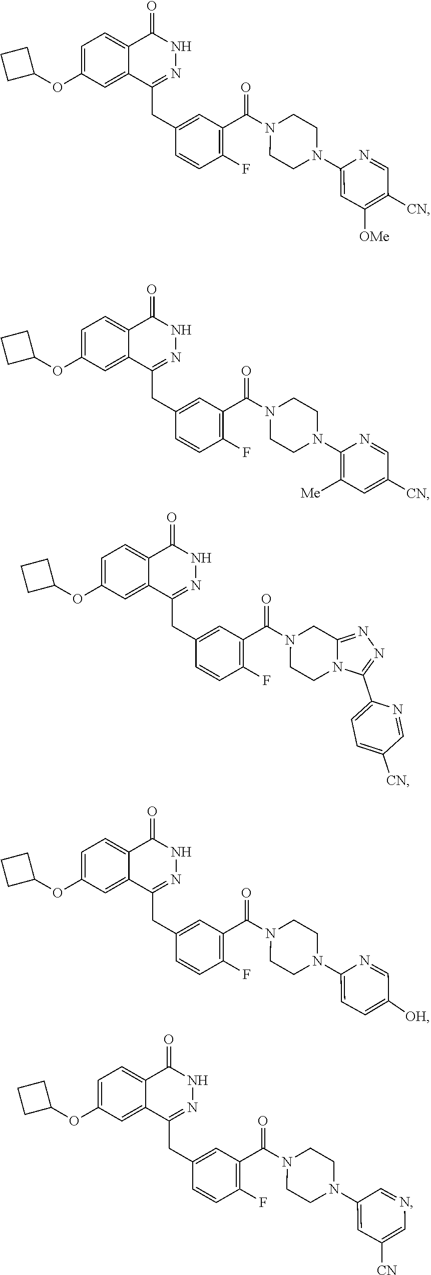 [SMILES: COc1cc(N2CCN(C(=O)c3cc(Cc4n[nH]c(=O)c5ccc(OC6CCC6)cc45)ccc3F)CC2)ncc1C#N ; Cc1cc(C#N)cnc1N1CCN(C(=O)c2cc(Cc3n[nH]c(=O)c4ccc(OC5CCC5)cc34)ccc2F)CC1 ; N#Cc1ccc(-c2nnc3n2CCN(C(=O)c2cc(Cc4n[nH]c(=O)c5ccc(OC6CCC6)cc45)ccc2F)C3)nc1 ; O=C(c1cc(Cc2n[nH]c(=O)c3ccc(OC4CCC4)cc23)ccc1F)N1CCN(c2ccc(O)cn2)CC1 ; N#Cc1cncc(N2CCN(C(=O)c3cc(Cc4n[nH]c(=O)c5ccc(OC6CCC6)cc45)ccc3F)CC2)c1]

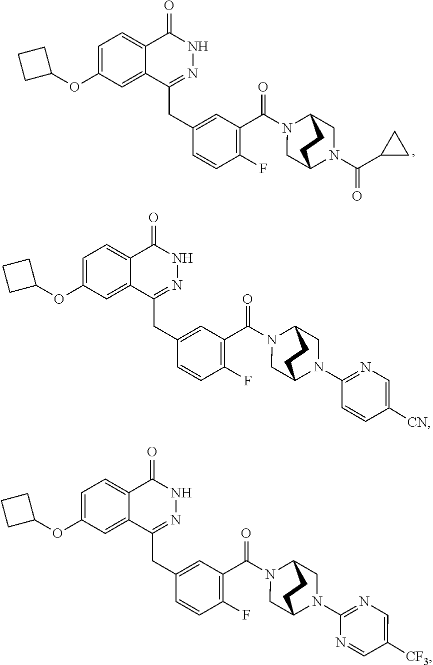

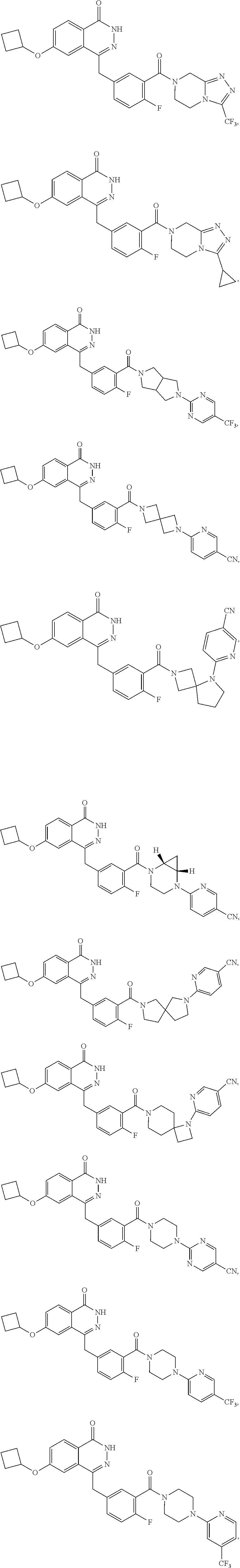

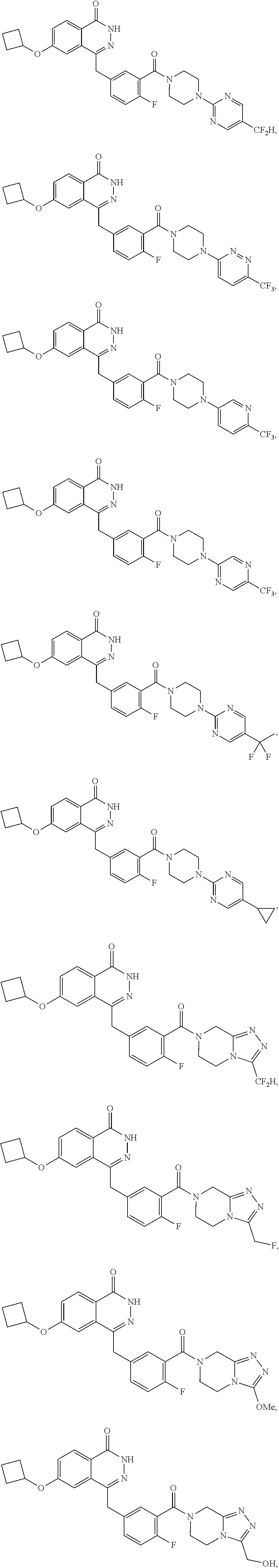

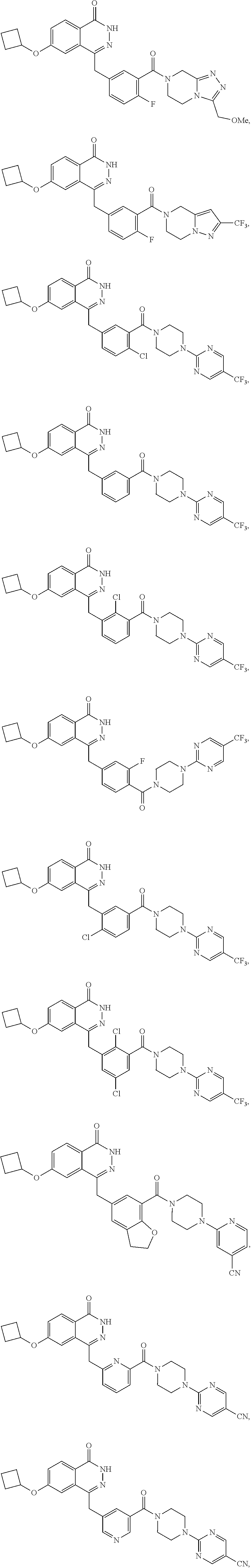

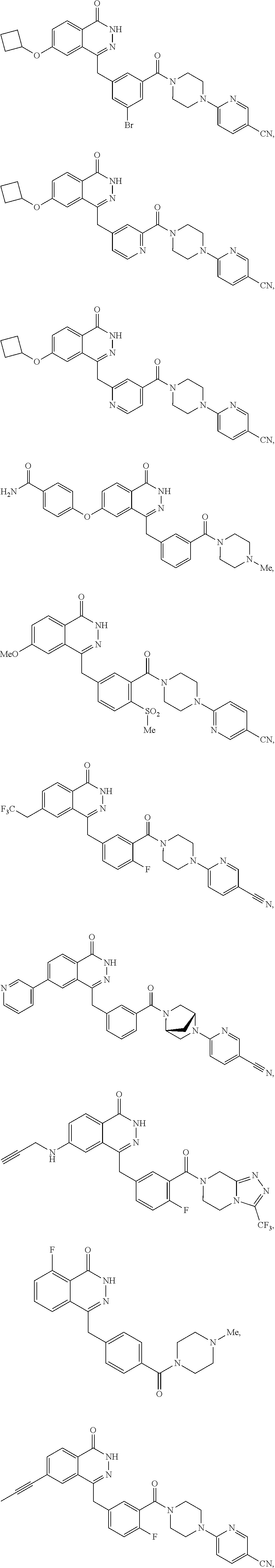

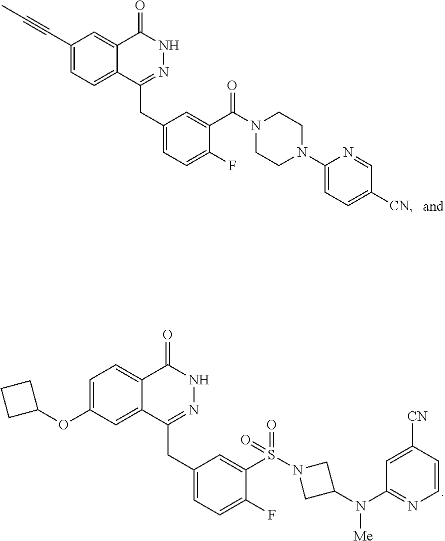 [SMILES: CC#Cc1ccc2c(Cc3ccc(F)c(C(=O)N4CCN(c5ccc(C#N)cn5)CC4)c3)n[nH]c(=O)c2c1 ; CN(c1cc(C#N)ccn1)C1CN(S(=O)(=O)c2cc(Cc3n[nH]c(=O)c4ccc(OC5CCC5)cc34)ccc2F)C1]

In some embodiments, the compound, and stereoisomers and pharmaceutically acceptable salt thereof, is selected from the group consisting of

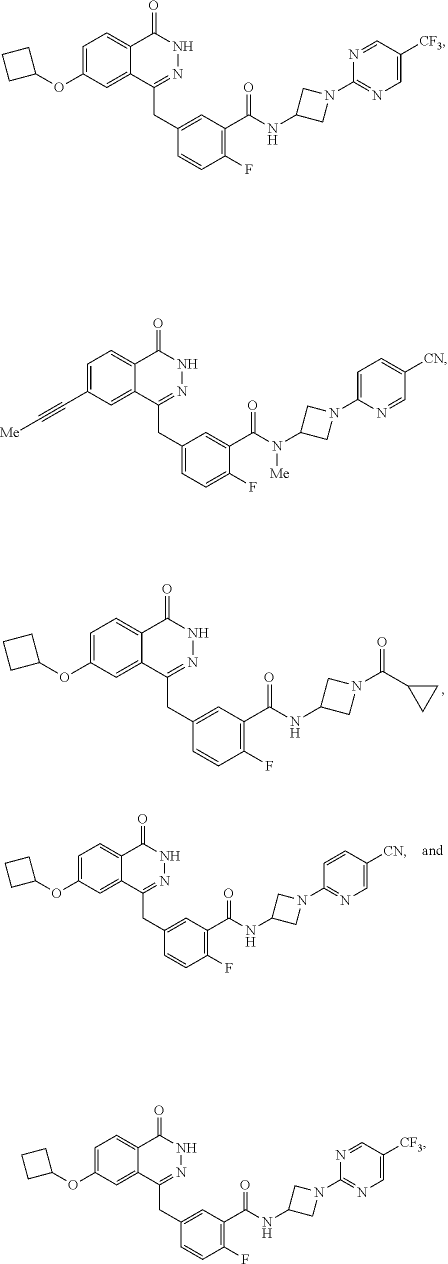 [SMILES: O=C(NC1CN(c2ncc(C(F)(F)F)cn2)C1)c1cc(Cc2n[nH]c(=O)c3ccc(OC4CCC4)cc23)ccc1F ; CC#Cc1ccc2c(=O)[nH]nc(Cc3ccc(F)c(C(=O)N(C)C4CN(c5ccc(C#N)cn5)C4)c3)c2c1 ; O=C(NC1CN(C(=O)C2CC2)C1)c1cc(Cc2n[nH]c(=O)c3ccc(OC4CCC4)cc23)ccc1F ; N#Cc1ccc(N2CC(NC(=O)c3cc(Cc4n[nH]c(=O)c5ccc(OC6CCC6)cc45)ccc3F)C2)nc1 ; O=C(NC1CN(c2ncc(C(F)(F)F)cn2)C1)c1cc(Cc2n[nH]c(=O)c3ccc(OC4CCC4)cc23)ccc1F]

In some embodiments, the compound, and stereoisomers and pharmaceutically acceptable salt thereof, is selected from the group consisting of

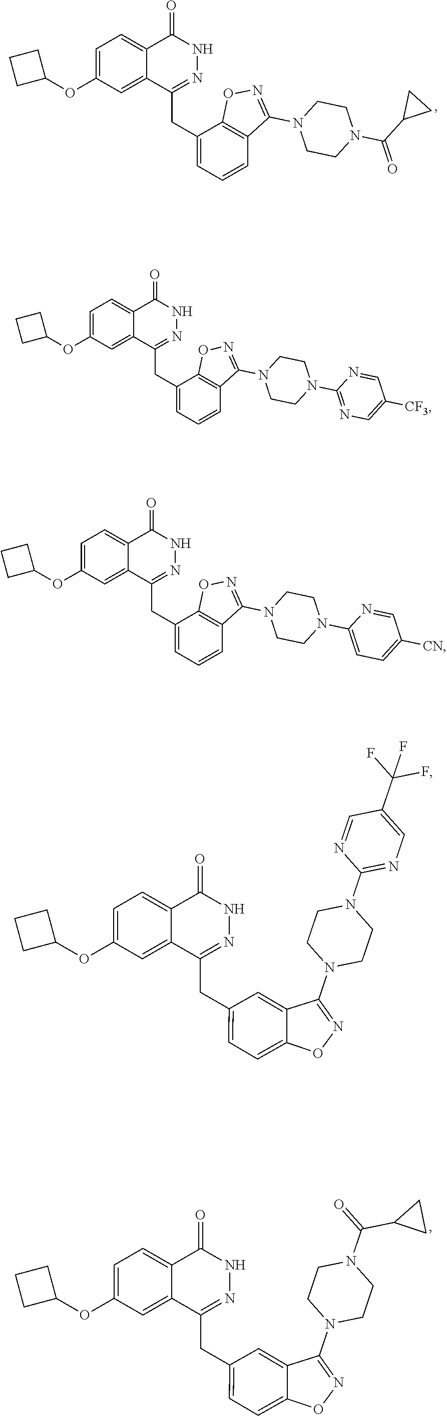 [SMILES: O=C(C1CC1)N1CCN(c2noc3c(Cc4n[nH]c(=O)c5ccc(OC6CCC6)cc45)cccc23)CC1 ; O=c1[nH]nc(Cc2cccc3c(N4CCN(c5ncc(C(F)(F)F)cn5)CC4)noc23)c2cc(OC3CCC3)ccc12 ; N#Cc1ccc(N2CCN(c3noc4c(Cc5n[nH]c(=O)c6ccc(OC7CCC7)cc56)cccc34)CC2)nc1 ; O=c1[nH]nc(Cc2ccc3onc(N4CCN(c5ncc(C(F)(F)F)cn5)CC4)c3c2)c2cc(OC3CCC3)ccc12 ; O=C(C1CC1)N1CCN(c2noc3ccc(Cc4n[nH]c(=O)c5ccc(OC6CCC6)cc45)cc23)CC1]

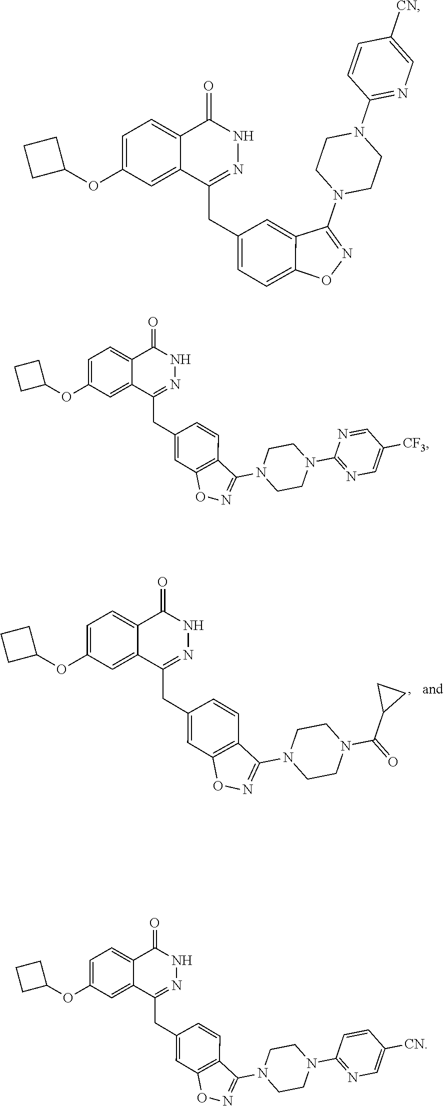 [SMILES: N#Cc1ccc(N2CCN(c3noc4ccc(Cc5n[nH]c(=O)c6ccc(OC7CCC7)cc56)cc34)CC2)nc1 ; O=c1[nH]nc(Cc2ccc3c(N4CCN(c5ncc(C(F)(F)F)cn5)CC4)noc3c2)c2cc(OC3CCC3)ccc12 ; O=C(C1CC1)N1CCN(c2noc3cc(Cc4n[nH]c(=O)c5ccc(OC6CCC6)cc45)ccc23)CC1 ; N#Cc1ccc(N2CCN(c3noc4cc(Cc5n[nH]c(=O)c6ccc(OC7CCC7)cc56)ccc34)CC2)nc1]

In some embodiments, the compound, and stereoisomers and pharmaceutically acceptable salt thereof, is selected from the group consisting of

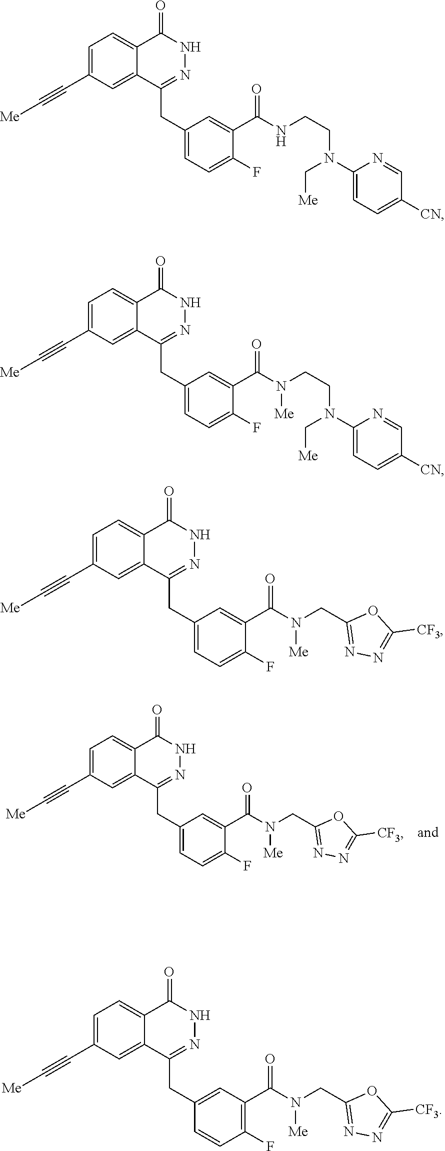 [SMILES: CC#Cc1ccc2c(=O)[nH]nc(Cc3ccc(F)c(C(=O)NCCN(CC)c4ccc(C#N)cn4)c3)c2c1 ; CC#Cc1ccc2c(=O)[nH]nc(Cc3ccc(F)c(C(=O)N(C)CCN(CC)c4ccc(C#N)cn4)c3)c2c1 ; CC#Cc1ccc2c(=O)[nH]nc(Cc3ccc(F)c(C(=O)N(C)Cc4nnc(C(F)(F)F)o4)c3)c2c1 ; CC#Cc1ccc2c(=O)[nH]nc(Cc3ccc(F)c(C(=O)N(C)Cc4nnc(C(F)(F)F)o4)c3)c2c1 ; "and" ; CC#Cc1ccc2c(=O)[nH]nc(Cc3ccc(F)c(C(=O)N(C)Cc4nnc(C(F)(F)F)o4)c3)c2c1]

Unless otherwise stated, structures depicted herein are also meant to include salts (e.g. pharmaceutically acceptable salts), solvates, hydrates, and isomers (e.g. stereoisomers) thereof. Accordingly, the present disclosure is directed to compounds of Formulae I, II, III, IV, V, VI, and VII and salts, solvates, hydrates, and isomers thereof. Moreover, reference to compounds of Formula I, II, III, IV, V, VI, and VII and stereoisomers and pharmaceutically acceptable salts thereof is considered to include reference to solvates, hydrates, and isomers (e.g. stereoisomers) of any thereof.

In some embodiments, the compound is a solvate, hydrate, or isomer (e.g. stereoisomer) of a compound of Formulae I, II, III, IV, V, VII, and VII and stereoisomers and pharmaceutically acceptable salts thereof.

Unless otherwise stated, structures depicted herein are also meant to include compounds which differ only in the presence of one or more isotopically enriched atoms. For example, compounds having the present structure except for the replacement of a hydrogen atom by deuterium or tritium, the replacement of a carbon atom by ¹³C or ¹⁴C, the replacement of a nitrogen atom by ¹⁵N, or the replacement of an oxygen atom by ¹⁷O or ¹⁸O are within the scope of the present disclosure. Such isotopically labeled compounds are useful as research or diagnostic tools.

### Methods of Synthesizing the Compounds

The compounds of Formulae I, II, III, IV, V, VI, and VII and stereoisomers and pharmaceutically acceptable salts thereof may be prepared by methods known in the art of organic synthesis. It is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999, which is hereby incorporated by reference in its entirety). These groups are removed at a convenient stage of the compound synthesis using methods that are readily apparent to those skilled in the art. The selection processes, as well as the reaction conditions and order of their execution, shall be consistent with the preparation of the disclosed compounds (e.g., Formulae I, II, II, IV, V, VI, and VII and stereoisomers and pharmaceutically acceptable salts thereof).

Those skilled in the art will recognize if a stereocenter exists in the compounds disclosed compounds (e.g. Formulae I, II, III, IV, V, VI, and VII and stereoisomers and pharmaceutically acceptable salts thereof). Accordingly, the present disclosure includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example. “Stereochemistry of Organic Compounds” by E. L. Eliel, S. H, Wilen, and L. N. Mander (Wiley-Interscience, 1994), which is hereby incorporated by reference in its entirety.

The compounds described herein may be made from commercially available starting materials or synthesized using known organic, inorganic, and/or enzymatic processes.

Compounds of Formulae I, II, III, IV, V, VI, and VII can be prepared according to procedures outlined in Schemes and Examples herein. In the Examples section, compounds of the present disclosure are further exemplified by specific examples. Unless otherwise specified, all temperatures were expressed in ° C. and all reactions are conducted at room temperature.

### Pharmaceutical Compositions

The compounds of Formulae I, II, III, IV, V, VI, and VII and stereoisomers and pharmaceutically acceptable salts thereof may be used on their own but will generally be administered in the form of a pharmaceutical composition in which the disclosed compound and stereoisomers and pharmaceutically acceptable salts thereof is in association with a pharmaceutically acceptable adjuvant, diluent or carrier. Conventional procedures for the selection and preparation of suitable pharmaceutical formulations are described in, for example. “Pharmaceuticals—The Science of Dosage Form Designs”. M. E. Aulton. Churchill Livingstone, 1988, which is hereby incorporated by reference in its entirety.

The present disclosure also provides a pharmaceutical composition comprising a compound of Formulae I, II, III, IV, V, VI, or VII, and stereoisomers and pharmaceutically acceptable salts thereof, and a pharmaceutically acceptable carrier.

The present disclosure also provides a compound of Formulae I, II, III, IV, V, VI, or VII, and stereoisomers and pharmaceutically acceptable salts thereof, for use in medicine.

The present disclosure also provides a pharmaceutical composition comprising a compound of Formulae I, II, III, IV, V, VI, or VII, and stereoisomers and pharmaceutically acceptable salts thereof, in association with a pharmaceutically acceptable adjuvant, diluent or carrier.

The present disclosure further provides a process for the preparation of a pharmaceutical composition of the present disclosure which comprises mixing a compound of Formulae I, II, III, IV, V, VI, or VII and stereoisomers and pharmaceutically acceptable salts thereof with a pharmaceutically acceptable adjuvant, diluent or carrier.

Depending on the mode of administration, the pharmaceutical composition will comprise from about 0.05 to about 99% w (percent by weight), more particularly from about 0.05 to about 80% w, still more particularly from about 0.10 to about 70% w, and even more particularly from about 0.10 to about 50% w, of active ingredient, all percentages by weight being based on total composition.

The pharmaceutical compositions may be administered topically (e.g. to the skin or to the lung and/or airways) in the form, e.g., of creams, solutions, suspensions, heptafluoroalkane (HFA) aerosols and dry powder formulations, for example, formulations in the inhaler device known as the Turbuhaler®; or systemically, e.g. by oral administration in the form of tablets, capsules, syrups, powders or granules; or by parenteral administration in the form of a sterile solution, suspension or emulsion for injection (including intravenous, subcutaneous, intramuscular, intravascular or infusion); or by rectal administration in the form of suppositories.

Dry powder formulations and pressurized HFA aerosols of the compounds of the present disclosure (including stereoisomers and pharmaceutically acceptable salts thereof) may be administered by oral or nasal inhalation. For inhalation, the compound is desirably finely divided. The finely divided compound preferably has a mass median diameter of less than 10 micrometres (μm), and may be suspended in a propellant mixture with the assistance of a dispersant, such as a C₈-C₂₀ fatty acid or salt thereof. (for example, oleic acid), a bile salt, a phospholipid, an alkyl saccharide, a perfluorinated or polyethoxylated surfactant, or other pharmaceutically acceptable dispersant.

The compounds of the present disclosure may also be administered by means of a dry powder inhaler. The inhaler may be a single or a multi dose inhaler, and may be a breath actuated dry powder inhaler.

One possibility is to mix the finely divided compound of the present disclosure with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatin capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

Another possibility is to process the compound as an amorphous dispersion in a polymer matrix such as hydroxypropyl methylcellulose (HPMC) or hydroxypropyl methylcellulose acetate succinate (HPMCAS). As the name suggests, spray-dried dispersions (SDDs) are obtained by dissolving drug and polymer in an organic solvent, atomizing the resulting solution into droplets, and evaporation to dried solid particles. SDDs are usually amenable for use a variety of final oral dosage forms, including capsules and tablets.

For oral administration the compound of the present disclosure may be admixed with an adjuvant or a carrier, for example, lactose, saccharose, sorbitol, mannitol; a starch, for example, potato starch, corn starch or amylopectin; a cellulose derivative; a binder, for example, gelatin or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatin, talcum and titanium dioxide. Alternatively, the tablet may be coated with a suitable polymer dissolved in a readily volatile organic solvent.

For the preparation of soft gelatin capsules, the compound of the present disclosure may be admixed with, for example, a vegetable oil or polyethylene glycol. Hard gelatin capsules may contain granules of the compound using either the above-mentioned excipients for tablets. Also liquid or semisolid formulations of the compound of the present disclosure may be filled into hard gelatin capsules.

Liquid preparations for oral application may be in the form of syrups or suspensions, for example, solutions containing the compound of the present disclosure, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

### Methods of Treatment

The terms “treat,” “treating,” or “treatment”, as used herein, refer to any indicia of success in the amelioration of a disorder (such as injury, disease pathology, or condition), including any objective or subjective parameter such as abatement; remission; diminishing of symptoms or making the disorder more tolerable to the subject; slowing or stopping the rate of degeneration, decline, or development; slowing the progression of disorder; making the final point of degeneration less debilitating; improving a subject's physical or mental well-being; or relieving or causing regression of the disorder. The treatment of symptoms, including the amelioration of symptoms, can be based on objective or subjective parameters, which may include the results of a physical examination, a neuropsychiatric exam, and/or a psychiatric evaluation. Certain methods and uses disclosed herein may treat cancer by, for example, causing remission of cancer, slowing the rate of growth of cancer cells, slowing the rate of spread of cancer cells, reducing metastasis, or reducing the growth of metastatic tumors, reducing the size of one or more tumors, reducing the number of one or more tumors, or any combinations thereof.

The terms “administered”, “administration”, or “administering”, as used herein, refers to either directly administering a disclosed compound (and stereoisomers and pharmaceutically acceptable salts thereof) or a composition to a subject, including an animal, in need of treatment by bringing such individual in contact with, or otherwise exposing such individual to, such compound.

As used herein, the term “subject” encompasses mammals and non-mammals. Examples of mammals include, but are not limited to, any member of the class Mammalia: humans, non-human primates such as chimpanzees, and other apes and monkey species: farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the present disclosure, the mammal is a human.

A “patient” is a mammal, e.g., a human, mouse, rat, guinea pig, dog, cat, horse, cow, pig, or non-human primate, such as a monkey, chimpanzee, baboon or rhesus. “Patient” includes both humans and animals.

“Inhibition”, as used herein, refers to reducing the activity of or complete inhibition of the target, activity may include reducing the activity of a component of a pathway to a level that is still detectable. Full inhibition may include stopping all activity of a component of a pathway (such as stopping the activity of PARP7), or reducing the activity of a component of a pathway to a level below detection. Inhibition of a component of a pathway may be measured directly or indirectly, using any methods known in the art. “Selective inhibition of PARP7”, as used herein, refers to wherein in vitro IC50 for PARP7 activity is less than about 10-fold compared to in vitro IC50 for other PARP family member activity in similar bio-assay formats, particularly PARP1 and PARP2. In some embodiments, the compound of Formulae I, II, III, IV, V, VI, and VII and stereoisomers and pharmaceutically acceptable salts thereof selectively inhibits PARP7.

The term “disorder”, as used herein, refers to and is used interchangeably with the terms disease, condition, or illness, unless otherwise indicated.

The term “disorder”, as used herein, refers to and is used interchangeably with the terms disease, condition, or illness, unless otherwise indicated.

In some embodiments, the present disclosure provides compounds which are suitable for use in the treatment of one or more disorders which are linked to PARP7 (e.g. the overexpression of PARP). “Responsive to inhibition of PARP7”, as used herein, refers to disorders expected to improve with administration of inhibiting doses of the compound. Cancer is a disorder that may be responsive to treatment by administration of the compound. Some specific cancers responsive to treatment by administration of the compound can be identified by analysis of in vitro expression levels and abnormal activity of PARP7 in cancer cell lines. Additionally, in vitro cell viability assays can be used to identify specific cancer cell lines that are responsive to treatment by administration of the compound. Cancer cell lines responsive to PARP7 inhibitor compounds in vitro are predicted to be responsive to PARP7 inhibitor compounds in vivo, for example in xenograft models for cancer growth inhibition in mice. Also, some cancers may be responsive to PARP7 inhibitors indirectly by activating the immune system of the patient rather than by causing death of the cancer cells directly. Additional methods may be used to identify PARP7-responsive cancers such tumor-specific mutations in particular genes, tumor gene expression patterns as well as biomarkers in the blood for example circulating tumor DNA, RNA or proteins.

The compounds of Formulae I, II, III, IV, V, VI, and VII and stereoisomers and pharmaceutically acceptable salts thereof have activity as pharmaceuticals, as discussed herein.

The present disclosure provides a compound of Formulae I, II, III, IV, V, VI, or VII, and stereoisomers and pharmaceutically acceptable salts thereof, or a pharmaceutical composition thereof for use in the treatment of a disorder in a subject in need thereof.

The present disclosure provides a compound of Formulae I, II, III, IV, V, VI, or VII, and stereoisomers and pharmaceutically acceptable salts thereof, or a pharmaceutical composition thereof for the treatment of a disorder in a subject in need thereof.

The present disclosure provides a compound of Formulae I, II, III, IV, V, VI, or VII, and stereoisomers and pharmaceutically acceptable salts thereof, or a pharmaceutical composition thereof, for use in the treatment of a disorder that is responsive to inhibition of PARP7.

The present disclosure provides a compound of Formulae I, II, III, IV, V, VI, or VII, and stereoisomers and pharmaceutically acceptable salts thereof, or a pharmaceutical composition thereof, in the treatment of a disorder that is responsive to inhibition of PARP7.

The present disclosure provides a compound of Formulae I, II, III, IV, V, VI, or VII, and stereoisomers and pharmaceutically acceptable salts thereof, or a pharmaceutical composition thereof, for use in the manufacture of a medicament for the treatment of a disorder that is responsive to inhibition of PARP7.

The present disclosure also provides a method of treating a disorder in a subject in need thereof, wherein the disorder is mediated by PARP7, comprising administering to the subject a compound of Formulae I, II, III, IV, V, VI, or VII, and stereoisomers and pharmaceutically acceptable salts thereof, or a pharmaceutical composition thereof.

In some embodiments, the disorder is selected from the group consisting of cancer, cardiovascular disorder, neurological disorder, inflammatory disorder, autoimmune disorder, and infectious disease.

In some embodiments, wherein the disorder is cancer. In some embodiments, the cancer is of solid organ origin or of hematopoietic origin.

In some embodiments, the disorder is cancer, the cancer is of solid organ origin, and the solid organ is selected from the group consisting of the brain, breast, colon, endometrium, esophagus, head and neck, upper gastrointestinal tract, respiratory tract, lung, kidney, liver, lower gastrointestinal tract, small intestine, large intestine, ovary, pancreas, prostate, stomach, testes, and urinary tract. In some embodiments, the disorder is cancer and the cancer is adenocarcinoma. In some embodiments, the disorder is non-small cell lung cancer. In some embodiments, the cancer is squamous cell carcinoma of the lung (SCCL).

In some embodiments, the disorder is cancer, and the cancer is leukemia or lymphoma. In some embodiments, the leukemia is acute lymphocytic leukemia (ALL), acute myelogenous leukemia (AML), chronic lymphocytic leukemia (CLL), or chronic myelogenous leukemia (CML). In some embodiments, the lymphoma is Hodgkin's lymphoma, non-Hodgkin's lymphoma, multiple myeloma, B-cell lymphoma, diffuse large B-cell lymphoma (DLBCL), chronic lymphocyteic lymphoma (CLL), T-cell lymphoma, hairy cell lymphoma, or Burkett's lymphoma.

In some embodiments, the disorder is cancer and the cancer is selected from the group consisting of cancer of the bladder, bone cancer, cancer of the cervix, cancer of the epithelium, cancer of the gallbladder, cancer of the rectum, skin cancer, thyroid cancer, and cancer of the uterus.

In some embodiments, a compound of Formulae I, II, III, IV, V, VI, or VII, and stereoisomers and pharmaceutically acceptable salts thereof, is administered to a subject in need thereof to inhibit a component of the PARP7 pathway. In certain embodiments, the compound or stereoisomer or a pharmaceutically acceptable salt thereof, is administered as a pharmaceutical composition, as described herein.

In some embodiments, inhibition of a component of the PARP7 pathway is measured directly, for example by measuring the product of a reaction catalyzed by a PARP7 pathway component. Inhibition of PARP7 activation may in some embodiments be demonstrated by western blotting and quantitatively assessing the levels of full length and cleaved PARP7 proteins from a cell line treated with compounds in vitro or in vivo.

In some embodiments, inhibition of a component of the PARP7 pathway is measured indirectly, for example by measuring the level of expression of one or more genes that are regulated by PARP7. The inhibition of a component of the PARP7 pathway, such as inhibition of catalytic activity (MARylation), may modulate the expression of one or more genes that are regulated by PARP7, for example IFN-β. The transcription levels may be assessed, for example, by transcriptomic analysis, including but not limited to q-PCR. Modulation of one, two, three, four, five, or more genes may indicate inhibition of PARP activation. This evaluation of endogenous IFN-β gene expression may be assessed in cell lines (such as CT26 cell lines) or primary cells (such as fibroblasts of mouse, rat, or human origin). In some embodiments, the gene transcription levels of IFN-β are evaluated. Inhibition of PARP7 activation may in some embodiments be demonstrated by detection of IFN-β secreted by cells treated with compounds in vitro or in vivo.

### Example Embodiments

Embodiment I-1. A compound of Formula I:

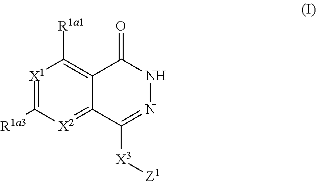

and stereoisomers and pharmaceutically acceptable salts thereof, wherein:
X¹ is —N— or —CR^{1a2}—;
X² is —N— or CR^{1a4}—;
R^{1a1}, R^{1a2}, R^{1a3}, and R^{1a4} are independently H, halo, —OH, C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆-alkynyl, —O-cycloalkyl, —O-heterocyclyl, —O-aryl, —O-heteroaryl, —SO₂-cycloalkyl, —NR^{1L1}R^{1L2}, or C₂₋₆alkynyl-NR^{1L3}R^{1L4};
wherein if X¹ is —CR^{1a2}— and X² is —CR^{1a4}—, then at least one of R^{1a1}, R^{1a2}, R^{1a3}, and R^{1a4} is not H;
wherein each C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, or —SO₂-cycloalkyl of R^{1a1}, R^{1a2}, R^{1a3}, and R^{1a4} is unsubstituted or substituted with one or more substituents independently selected from the group consisting of halo, —OH, —CN, C₁₋₆alkyl, C₁₋₆haloalkyl, cycloalkyl, halocycloalkyl, —O—C₁₋₆alkyl, and —C(O)NH₂;
R^{1L1}, R^{1L2}, R^{1L3}, and R^{1L4} are independently H, C₁₋₆alkyl, C₂₋₆alkynyl, or cycloalkyl, wherein each C₁₋₆alkyl, C₂₋₆alkynyl, or cycloalkyl of R^{1L1}, R^{12L}, R^{1L3}, and R^{1L4} is unsubstituted or substituted with one or more substituents independently selected from the group consisting of halo, C₁₋₆alkyl, C₁₋₆haloalkyl, cycloalkyl, and halocycloalkyl;
X³ is —O—, —NR^{1b1}—, or —CR^{1b2}R^{1b3}—, wherein R^{1b1}, R^{1b2}, and R^{1b3} are independently H or C₁₋₆alkyl;
Z¹ is

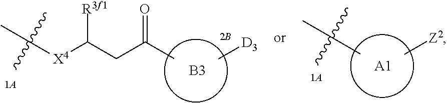

wherein a bond marked 1A is to X³ and a bond marked 2B is to D3, D4, D5, or D6;
R^{3f1} is H or C₁₋₆alkyl;
X⁴ is —O—, —NR^{3f2}—, or —CR^{3f3}R^{3f4}—, wherein R^{3f2}, R^{3f3}, and R^{3f4} are independently selected from H and C₁₋₆alkyl;
A1 is

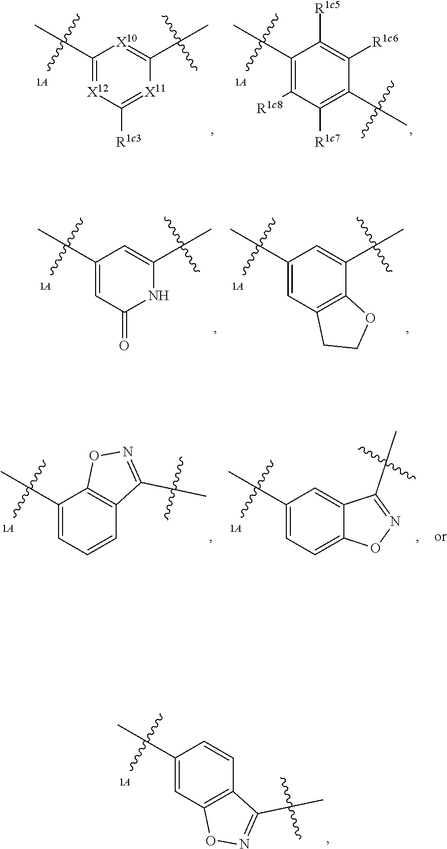

wherein a bond marked 1A is to X³;
X¹⁰ is —N— or —CR^{1c1}—, X¹¹ is —N— or —CR^{1c2}—, and X¹² is —N— or —CR^{1c4}—;
R^{1c1}, R^{1c2}, R^{1c3}, R^{1c4}, R^{1c5}, R^{1c6}, R^{1c7}, and R^{1c8} are independently H, halo, —CN, —SO₂CH₃, —SO₂NH₂, or —NHSO₂CH₃;
Z² is

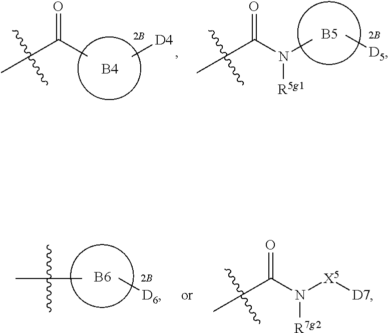

wherein a bond marked 2B is to D3, D4, D5, or D6;
X⁵ is —CH(R^{7g3})— or —CH(R^{7g4})CH₂N(R^{7g5})—;
R^{5g1}, R^{7g2}, R^{7g3}, R^{7g4}, and R^{7g5} are independently H or C₁₋₆alkyl;
B3, B4, B5, and B6 are independently a 3 to 8-membered monocyclic heterocyclediyl, a 7 to 18-membered polycyclic heterocyclediyl, or a 7 to 18-membered spirocyclic heterocyclediyl;
wherein the 3 to 8-membered monocyclic heterocyclediyl, 7 to 18-membered polycyclic heterocyclediyl, or 7 to 18-membered spirocyclic heterocyclediyl of B3, B4, B5, and B6 is unsubstituted or substituted with one or more substituents independently selected from the group consisting of halo, C₁₋₆alkyl, and oxo;
provided that B4 is not

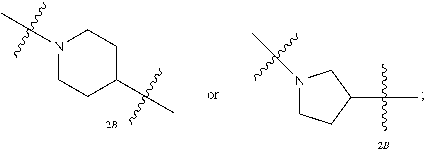 [SMILES: *C1CCN(*)CC1 ; *C1CCN(*)C1]

D3, D4, D5, D6, and D7 are independently C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, —C(O)-heteroaryl, —N(R^{1D1})(R^{1D2}), —C(O)N(R^{1D3})(R^{1D4}), or —N(R^{1D5})C(O)R^{1D6};
wherein the C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, or —C(O)-heteroaryl of D3, D4, D5, D6, and D7 is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{1D10})₂, wherein each R^{1D10} is independently H or C₁₋₆alkyl;
R^{1D1}, R^{1D3}, and R^{1D5} are independently H or C₁₋₆alkyl;
R^{1D2} is aryl or heteroaryl, wherein the aryl or heteroaryl of R^{1D2} is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{1D11})₂, wherein each R^{1D11} is independently H or C₁₋₆alkyl; and
R^{1D4} and R^{1D6} are independently C₁₋₆alkyl, cycloalkyl, heterocyclyl, aryl, or heteroaryl, wherein each C₁₋₆-alkyl, cycloalkyl, heterocyclyl, aryl, or heteroaryl of R^{1D4} and R^{1D6} is unsubstituted or substituted with one or more substituents independently selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{1D12})₂, wherein each R^{1D12} is independently H or C₁₋₆alkyl;
provided that if B4 is

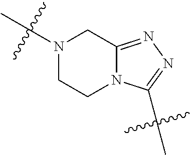 [SMILES: *c1nnc2n1CCN(*)C2]

and D4 is —CHF₂, —CF₂CH₃, or —CF₂CF₃, then R^{1a2} is not F, if X³ is —O—, B4 is

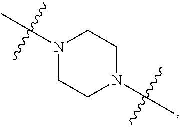 [SMILES: *N1CCN(*)CC1]

and D4 is —C(O)-aryl, then R^{1a4} is not Cl, and if D4 is

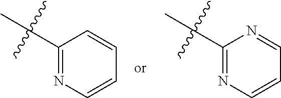 [SMILES: *c1ccccn1 ; *c1ncccn1]

and R^{1c2} is F, then R^{1a2} is not F.

Embodiment I-2. A compound of Formula II;

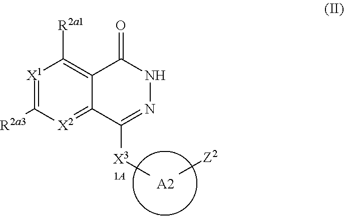

and stereoisomers and pharmaceutically acceptable salts thereof, wherein:
X¹ is —N— or —CR^{2a2}—;
X² is —N— or —CR^{2a4}—;
R^{2a1}, R^{2a2}, R^{2a3}, and R^{2a4} are independently H, halo, —OH, C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, —SO₂-cycloalkyl, —NR^{2L1}R^{2L2}, or C₂₋₆alkynyl-NR^{2L3}R^{2L4};
wherein if X¹ is —CR^{2a2}— and X² is —CR^{2a4}—, then at least one of R^{2a1}, R^{2a2}, R^{2a3}, and R^{2a4} is not H;
wherein each C₁₋₆alkyl, C₂₋₆alkenyl. C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, or —SO₂-cycloalkyl of R^{2a1}, R^{2a2}, R^{2a3}, and R^{2a4} is unsubstituted or substituted with one or more substituents independently selected from the group consisting of halo, —OH, —CN, C₁₋₆alkyl, C₁₋₆haloalkyl, cycloalkyl, halocycloalkyl, —O—C₁₋₆alkyl, and —C(O)NH₂;
R^{2L1}, R^{2L2}, R^{2L3}, and R^{2L4} are independently H, C₁₋₆alkyl, C₂₋₆alkynyl, or cycloalkyl, wherein each C₁₋₆alkyl, C₂₋₆alkynyl, or cycloalkyl of R^{2L1}, R^{2L2}, R^{2L3}, and R^{2L4} is unsubstituted or substituted with one or more substituents independently selected from the group consisting of halo, C₁₋₆alkyl, C₁₋₆haloalkyl, cycloalkyl, and halocycloalkyl;
X³ is —O—, —NR^{2b1}—, or —CR^{2b2}R^{2b3}—, wherein R^{2b1}, R^{2b2}, and R^{2b3} are independently H or C₁₋₆alkyl;
A2 is

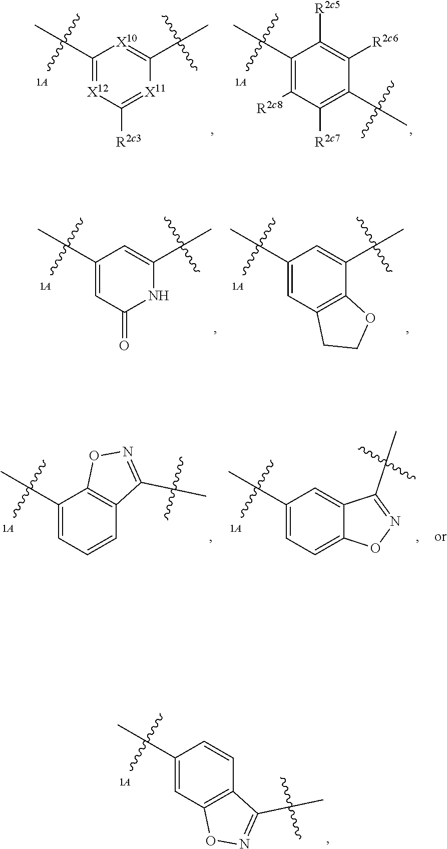

wherein a bond marked 1A is to X³;
X¹⁰ is —N— or —CR^{2c1}—, X¹¹ is —N— or —CR^{2c2}—, and X¹² is —N— or —CR^{2c4}—;
R^{2c1}, R^{2c2}, R^{2c3}, R^{2c4}, R^{2c5}, R^{2c6}, R^{2c7} and R^{2c8} are independently H, halo, —CN, —SO₂CH₃, —SO₂NH₂, or —NHSO₂CH₃;
Z² is

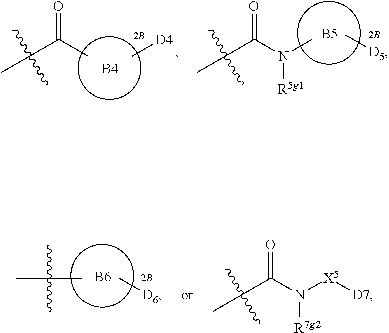

wherein a bond marked 2B is to D4, D5, or D6;
X⁵ is —CH(R^{7g3})— or —CH(R^{7g4})CH₂N(R^{7g5})—;
R^{5g1}, R^{7g2}, R^{7g3}, R^{7g4}, and R^{7g5} are independently H or C₁₋₆alkyl;
B4, B5, and B6 are independently a 3 to 8-membered monocyclic heterocyclediyl, a 7 to 18-membered polycyclic heterocyclediyl, or a 7 to 18-membered spirocyclic heterocyclediyl;
wherein the 3 to 8-membered monocyclic heterocyclediyl, 7 to 18-membered polycyclic heterocyclediyl, or 7 to 18-membered spirocyclic heterocyclediyl of B4, B5, and B6 is unsubstituted or substituted with one or more substituents independently selected from the group consisting of halo, C₁₋₆alkyl, and oxo;
provided that B4 is not

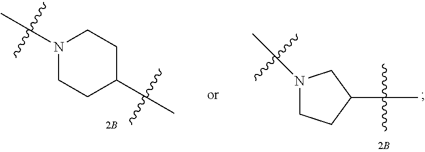 [SMILES: *C1CCN(*)CC1 ; *C1CCN(*)C1]

D4, D5, D6, and D7 are independently C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, —C(O)-heteroaryl, —N(R^{2D1})(R^{2D2}), —C(O)N(R^{2D3})(R^{2D4}), or —N(R^{2D5})C(O)R^{2D6};
wherein the C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, or —C(O)-heteroaryl of D4, D5, D6, and D7 is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{2D10)}₂, wherein each R^{2D10} is independently H or C₁₋₆alkyl;
R^{2D1}, R^{2D3}, and R^{2D5} are independently H or C₁₋₆alkyl;
R^{2D2} is aryl or heteroaryl, wherein the aryl or heteroaryl of R^{2D2} is unsubstituted or substituted with one or more substituents selected from the group consisting of halo, —CN, C₁₋₆alkyl, C₁₋₆haloalkyl, cycloalkyl, —OH, —C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{2D11})₂, wherein each R^{2D11} is independently H or C₁₋₆alkyl; and
R^{2D4} and R^{2D6} are independently C₁₋₆alkyl, cycloalkyl, heterocyclyl, aryl, or heteroaryl, wherein each C₁₋₆alkyl, cycloalkyl, heterocyclyl, aryl, or heteroaryl of R^{2D4} and R^{2D6} is unsubstituted or substituted with one or more substituents independently selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{2D12})₂, wherein each R^{2D12} is independently H or C₁₋₆alkyl;
provided that if B4 is

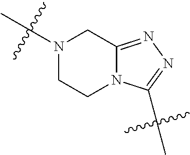 [SMILES: *c1nnc2n1CCN(*)C2]

and D4 is —CHF₂, —CF₂CH₃, or —CF₂CF₃, then R^{2a2} is not F, if X³ is —O—, B4 is

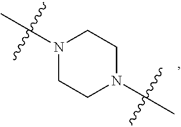 [SMILES: *N1CCN(*)CC1]

and D4 is C(O)-aryl, then R^{2a4} is not Cl, and if D4 is

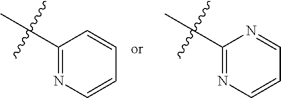 [SMILES: *c1ccccn1 ; *c1ncccn1]

and R^{2c2} is F, then R^{2a2} is not F.

Embodiment I-3. A compound of Formula III:

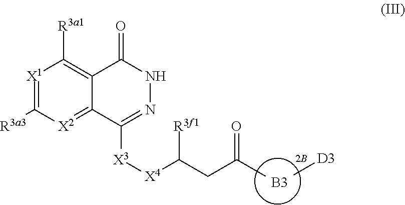

and stereoisomers and pharmaceutically acceptable salts thereof, wherein:
X¹ is —N— or —CR^{3a2}—;
X² is —N— or —CR^{3a4}—;
R^{3a1}, R^{3a2}, R^{3a3}, and R^{3a4} are independently H, halo, —OH, C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, —SO₂-cycloalkyl, —NR^{3L1}R^{3L2}, or C₂₋₆alkynyl-NR^{3L3}R^{3L4};
wherein if X¹ is —CR^{3a2}— and X² is —CR^{3a4}—, then at least one of R^{3a1}, R^{3a2}, R^{3a3}, and R^{3a4} is not H;
wherein each C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆-alkenyl, —O—C₂₋₆alkynyl, —O-cycloalkyl, —O-heterocyclyl, —O-aryl, —O-heteroaryl, or —SO₂-cycloalkyl of R^{3a1}, R^{3a2}, R^{3a3}, and R^{3a4} is unsubstituted or substituted with one or more substituents independently selected from the group consisting of halo, —OH, —CN, C₁₋₆alkyl, C₁₋₆haloalkyl, cycloalkyl, halocycloalkyl, —O—C₁₋₆alkyl, and —C(O)NH₂;
R^{3L1}, R^{3L2}, R^{3L3}, and R^{3L4} are independently H, C₁₋₆-alkyl, C₂₋₆alkynyl, or cycloalkyl, wherein each C₁₋₆alkyl, C₂₋₆alkynyl, or cycloalkyl of R^{3L1}, R^{3L2}R^{3L3} and R^{3L4} is unsubstituted or substituted with one or more substituents independently selected from the group consisting of halo, C₁₋₆alkyl, C₁₋₆haloalkyl, cycloalkyl, and halocycloalkyl;
X³ is —O—, —NR^{3b1}—, or —CR^{3b2}R^{3b3}—, wherein R^{3b1}, R^{3b2}, and R^{3b3} are independently H or C₁₋₆alkyl;
R^{3f1} is H or C₁₋₆alkyl;
X⁴ is —O—, —NR^{3f2}—, or —CR^{3f3}R^{3f4}—, wherein R^{3f2}, R^{3f3}, and R^{3f4} are independently selected from H and C₁₋₆alkyl;
B3 is a 3 to 8-membered monocyclic heterocyclediyl, a 7 to 18-membered polycyclic heterocyclediyl, or a 7 to 18-membered spirocyclic heterocyclediyl;
wherein the 3 to 8-membered monocyclic heterocyclediyl, 7 to 18-membered polycyclic heterocyclediyl, or 7 to 18-membered spirocyclic heterocyclediyl of B3 is unsubstituted or substituted with one or more substituents selected from the group consisting of halo, C₁₋₆alkyl, and oxo;
D3 is C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, —C(O)-heteroaryl, —N(R^{3D1})(R^{3D2}), —C(O)N(R^{3D3})(R^{3D4}), or —N(R^{3D5})C(O)R^{3D6};
wherein the C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, or —C(O)-heteroaryl of D3 is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-C₁₋₆alkyl, C₁₋₆alkyl-O—C₁₋₆haloalkyl, —C(O)-cycloalkyl, and —C(O)N(R^{3D10})₂, wherein each R^{3D10} is independently H or C₁₋₆alkyl;
R^{3D1}, R^{3D3}, and R^{3D5} are independently H or C₁₋₆alkyl;
R^{3D2} is aryl or heteroaryl, wherein the aryl or heteroaryl of R^{3D2} is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{3D11})₂, wherein each R^{3D11} is independently H or C₁₋₆alkyl; and
R^{3D4} and R^{3D6} are independently C₁₋₆alkyl, cycloalkyl, heterocyclyl, aryl, or heteroaryl, wherein each C₁₋₆alkyl, cycloalkyl, heterocyclyl, aryl, or heteroaryl of R^{3D4} and R^{3D6} is unsubstituted or substituted with one or more substituents independently selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{3D12})₂, wherein each R^{3D12} is independently H or C₁₋₆alkyl.

Embodiment I-4. The compound of embodiment I-2, and stereoisomers and pharmaceutically acceptable salts thereof, wherein the compound is of Formula IV:

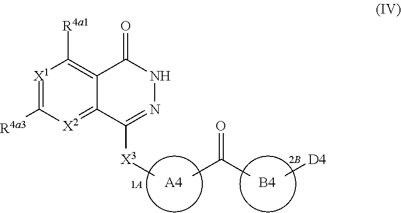

X¹ is —N— or —CR^{4a2}—;
X² is —N— or —CR^{4a4}—;
R^{4a1}, R^{4a2}, R^{4a3}, and R^{4a4} are independently H, halo, —OH, C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, —SO₂-cycloalkyl, —NR^{4L1}R^{4L2}, or C₂₋₆alkynyl-NR^{4L3}R^{4L4};
wherein if X¹ is —CR^{4a2}— and X² is —CR^{4a4}—, then at least one of R^{4a1}, R^{4a2}, R^{4a3}, and R^{4a4} is not H;
wherein each C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, or —SO₂-cycloalkyl of R^{4a1}, R^{4a2}, R^{4a3}, and R^{4a4} is unsubstituted or substituted with one or more substituents independently selected from the group consisting of halo, —OH, —CN, C₁₋₆alkyl, C₁₋₆haloalkyl, cycloalkyl, halocycloalkyl, —O—C₁₋₆alkyl, and —C(O)NH₂;
R^{4L1}, R^{4L2}, R^{4L3}, and R^{4L4} are independently H, C₁₋₆alkyl, C₂₋₆alkynyl, or cycloalkyl, wherein each C₁₋₆alkyl, C₂₋₆alkynyl, or cycloalkyl of R^{4L1}, R^{4L2}, R^{4L3}, and R^{4L4} is unsubstituted or substituted with one or more substituents independently selected from the group consisting of halo, C₁₋₆alkyl, C₁₋₆haloalkyl, cycloalkyl, and halocycloalkyl.
X³ is —O—. —NR^{4b1}—, or —CR^{4b2}R^{4b3}—, wherein R^{4b1}, R^{4b2}, and R^{4b3} are independently H or C₁₋₆alkyl;
A4 is

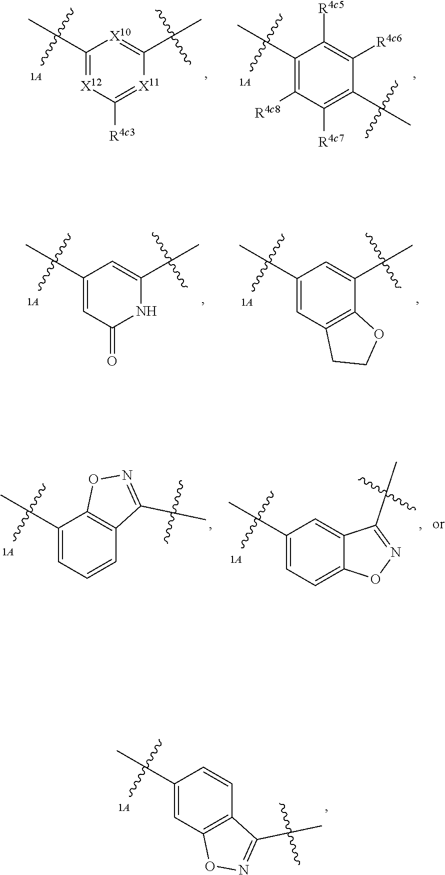

wherein a bond marked 1A is to X³;
X¹⁰ is —N— or —CR^{4c1}—, X¹¹ is —N— or —CR^{4c2}—, and X¹² is —N— or —CR^{4c4}—;
R^{4c1}, R^{4c2}, R^{4c3}, R^{4c4}, R^{4c5}, R^{4c6}, R^{4c7}, and R^{4c8} are independently H, halo, —CN, —SO₂CH₃, —SO₂NH₂, or —NHSO₂CH₃;
B4 is a 3 to 8-membered monocyclic heterocyclediyl, a 7 to 18-membered polycyclic heterocyclediyl, or a 7 to 18-membered spirocyclic heterocyclediyl;
wherein the 3 to 8-membered monocyclic heterocyclediyl, 7 to 18-membered polycyclic heterocyclediyl, or 7 to 18-membered spirocyclic heterocyclediyl of B4 is unsubstituted or substituted with one or more substituents selected from the group consisting of halo, C₁₋₆alkyl, and oxo;
provided that B4 is not

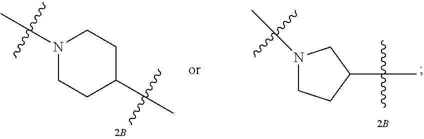 [SMILES: *C1CCN(*)CC1 ; *C1CCN(*)C1]

D4 is C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, —C(O)-heteroaryl, —N(R^{4D1})(R^{4D2}), —C(O)N(R^{4D3})(R^{4D4}), or —N(R^{4D5})C(O)R^{4D6};
wherein the C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, or —C(O)-heteroaryl of D4 is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{4D10})₂, wherein each R^{4D10} is independently H or C₁₋₆alkyl;
R^{4D1}, R^{4D3}, and R^{4D5} are independently H or C₁₋₆alkyl;
R^{4D2} is aryl or heteroaryl, wherein the aryl or heteroaryl of R^{4D2} is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{4D11})₂, wherein each R^{4D11} is independently H or C₁₋₆alkyl; and
R^{4D4} and R^{4D6} are independently C₁₋₆alkyl, cycloalkyl, heterocyclyl, aryl, or heteroaryl, wherein each C₁₋₆alkyl, cycloalkyl, heterocyclyl, aryl, or heteroaryl of R^{4D4} and R^{4D6} is unsubstituted or substituted with one or more substituents independently selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{4D12})₂, wherein each R^{4D12} is independently H or C₁₋₆alkyl;
provided that if B4 is

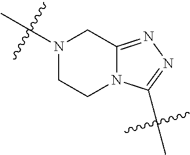 [SMILES: *c1nnc2n1CCN(*)C2]

and D4 is —CHF₂, —CF₂CH₃, or —CF₂CF₃, then R^{4a2} is not F, if X³ is —O—, B4 is

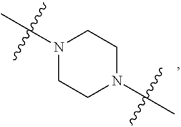 [SMILES: *N1CCN(*)CC1]

and D4 is —C(O)-aryl, then R is not Cl, and if D4 is

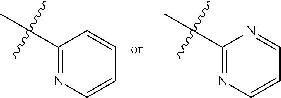 [SMILES: *c1ccccn1 ; *c1ncccn1]

and R^{4c2} is F, then R^{4a2} is not F.

Embodiment I-5. The compound of embodiment I-2, and stereoisomers and pharmaceutically acceptable salts thereof, wherein the compound is of Formula V:

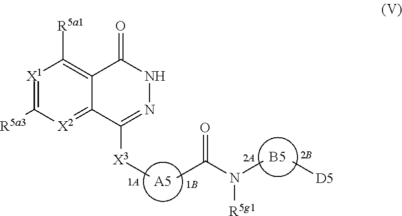

X¹ is —N— or —R^{5a2}—;
X² is —N— or —CR^{5a4}—;
R^{5a1}, R^{5a2}, R^{5a3}, and R^{5a4} are independently H, halo, —OH, C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, —SO₂-cycloalkyl, —NR^{5L1}R^{5L2}, or C₂₋₆alkynyl-NR^{5L3}R^{5L4};
wherein if X¹ is —CR^{5a2}— and X² is —CR^{5a4}—, then at least one of R^{5a1}, R^{5a2}, R^{5a3}, and R^{5a4} is not H;
wherein each C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, or —SO₂-cycloalkyl of R^{5a1}, R^{5a2}, R^{5a3}, and R^{5a4} is unsubstituted or substituted with one or more substituents independently selected from the group consisting of halo, —OH, —CN, C₁₋₆alkyl, C₁₋₆haloalkyl, cycloalkyl, halocycloalkyl, —O—C₁₋₆alkyl, and —C(O)NH₂;
R^{5L1}, R^{5L2}, R^{5L3}, and R^{5L4} are independently H, C₁₋₆alkyl, C₂₋₆alkynyl, or cycloalkyl, wherein each C₁₋₆alkyl, C₂₋₆alkynyl, or cycloalkyl of R^{5L1}, R^{5L2}, R^{5L3}, and R^{5L4} is unsubstituted or substituted with one or more substituents independently selected from the group consisting of halo, C₁₋₆alkyl, C₁₋₆haloalkyl, cycloalkyl, and halocycloalkyl;
X³ is —O—, —NR^{5b1}—, or —CR^{5b2}R^{5b3}—, wherein R^{5b1}, R^{5b2}, and R^{5b3} are independently H or C₁₋₆alkyl;
A5 is

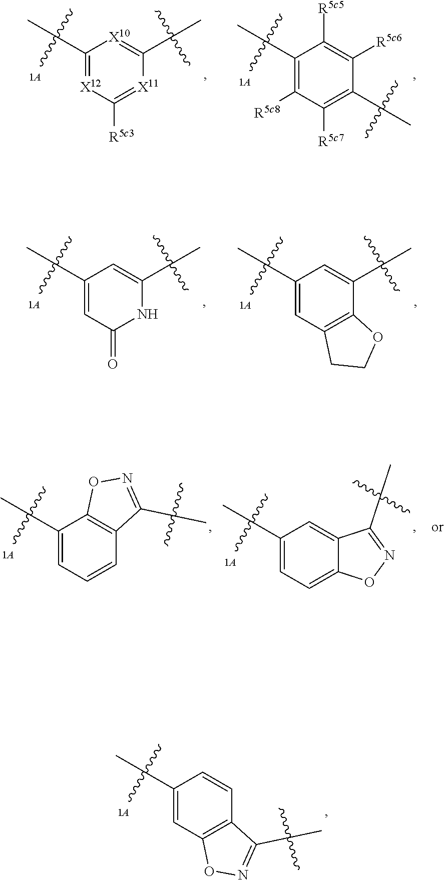

wherein a bond marked 1A is to X³;
X¹⁰ is —N— or —CR^{5c1}—, X¹¹ is —N— or —CR^{5c2}—, and X¹² is —N— or —CR^{5c4}—;
R^{5c1}, R^{5c2}, R^{5c3}, R^{5c4}, R^{5c5}, R^{5c6}, R^{5c7}, and R^{5c8} are independently H, halo, —CN, —SO₂CH₃, —SO₂NH₂, or —NHSO₂CH₃;
R^{5g1} is H or C₁₋₆alkyl;
B5 is a 3 to 8-membered monocyclic heterocyclediyl, a 7 to 18-membered polycyclic heterocyclediyl, or a 7 to 18-membered spirocyclic heterocyclediyl;
wherein the 3 to 8-membered monocyclic heterocyclediyl, 7 to 18-membered polycyclic heterocyclediyl, or 7 to 18-membered spirocyclic heterocyclediyl of B5 is unsubstituted or substituted with one or more substituents selected from the group consisting of halo, C₁₋₆alkyl, and oxo;
D5 is C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, —C(O)-heteroaryl, —N(R^{5L1})(R^{5B2}), —C(O)N(R^{5L3})(R^{5D4}), or —N(R^{5D5})C(O)R^{5D6};
wherein the C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, or —C(O)-heteroaryl of D5 is unsubstituted or substituted with one or more substituents selected from the group consisting of halo, —CN, C₁₋₆alkyl, C₁₋₆ haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{5D10})₂, wherein each R^{5D10} is independently H or C₁₋₆alkyl;
R^{5D1}, R^{5D3}, and R^{5D5} are independently H or C₁₋₆alkyl;
R^{5D2} is aryl or heteroaryl, wherein the aryl or heteroaryl of R^{5D2} is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-C₁₋₆haloalkyl, —C(O)-cycloalkyl, and —C(O)N(R^{5D11})₂, wherein each R^{5D11} is independently H or C₁₋₆alkyl; and
R^{5D4} and R^{5D6} are independently C₁₋₆alkyl, cycloalkyl, heterocyclyl, aryl, or heteroaryl, wherein each C₁₋₆alkyl, cycloalkyl, heterocyclyl, aryl, or heteroaryl of R^{5D4} and R^{5D6} is unsubstituted or substituted with one or more substituents independently selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{5D12})₂, wherein each R^{5D12} is independently H or C₁₋₆alkyl.

Embodiment I-6. The compound of embodiment I-2, and stereoisomers and pharmaceutically acceptable salts thereof, wherein the compound is of Formula VI:

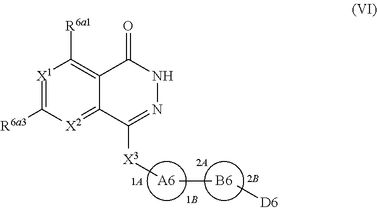

X¹ is —N— or —CR^{6a2}—;
X² is —N— or —CR^{6a4}—;
R^{6a1}, R^{6a2}, R^{6a3}, and R^{6a4} are independently H, halo, —OH, C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl. —O—C₂₋₆alkynyl, —O-cycloalkyl, —O-heterocyclyl, —O-aryl, —O-heteroaryl, —SO₂-cycloalkyl, —NR^{6L1}—R^{6L2}, or C₂₋₆alkynyl-NR^{6L3}R^{6L4};
wherein if X¹ is —CR^{6a2}— and X² is —CR^{6a4}—, then at least one of R^{6a1}, R^{6a2}, R^{6a3}, and R^{6a4} is not H;
wherein each C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, or —SO₂-cycloalkyl of R^{6a1}, R^{6a2}, R^{6a3}, and R^{6a4} is unsubstituted or substituted with one or more substituents independently selected from the group consisting of halo, —OH, —CN, C₁₋₆alkyl, C₁₋₆haloalkyl, cycloalkyl, halocycloalkyl, —O—C₁₋₆alkyl, and —C(O)NH₂;
R^{6L1}, R^{6L2}, R^{6L3}, and R^{6L4} are independently H, C₁₋₆alkyl, C₂₋₆alkynyl, or cycloalkyl, wherein each C₁₋₆alkyl, C₂₋₆alkynyl, or cycloalkyl of R^{6L1}, R^{6L2}, R^{6L3}, and R^{6L4} is unsubstituted or substituted with one or more substituents independently selected from the group consisting of halo, C₁₋₆alkyl, C₁₋₆haloalkyl, cycloalkyl, and halocycloalkyl;
X³ is —O—, —NR^{6b1}—, or —CR^{6b2}R^{6b3}—, wherein R^{6b1}, R^{6b2}, and R^{6b3} are independently H or C₁₋₆alkyl;
A6 is

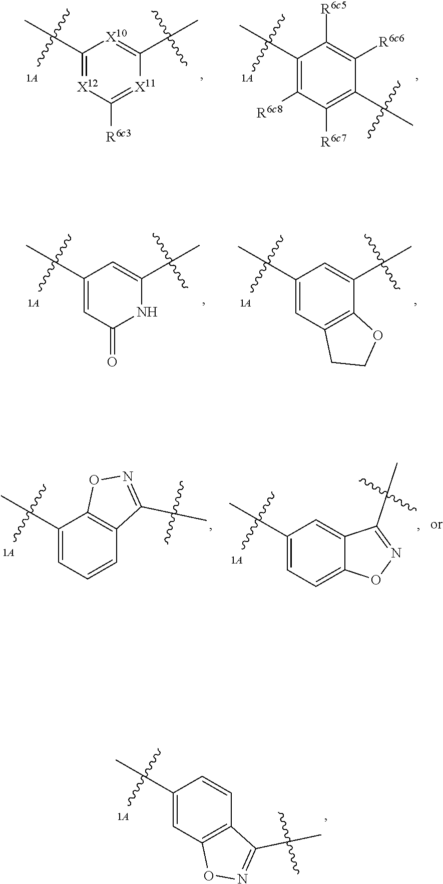

wherein a bond marked 1A is to X³;
X¹⁰ is —N— or —CR^{6c1}—, X¹¹ is —N— or —CR^{6c2}—, and X¹² is —N— or —CR^{6c4}—;
R^{6c1}, R^{6c2}, R^{6c3}, R^{6c4}, R^{6c5}, R^{6c6}, R^{6c7}, and R^{6c8} are independently H, halo, —CN, —SO₂CH₃, —SO₂NH₂, or —NHSO₂CH₃;
B6 is a 3 to 8-membered monocyclic heterocyclediyl, a 7 to 18-membered polycyclic heterocyclediyl, or a 7 to 18-membered spirocyclic heterocyclediyl;
wherein the 3 to 8-membered monocyclic heterocyclediyl, 7 to 18-membered polycyclic heterocyclediyl, or 7 to 18-membered spirocyclic heterocyclediyl of B6 is unsubstituted or substituted with one or more substituents selected from the group consisting of halo, C₁₋₆alkyl, and oxo;
D6 is C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, —C(O)-heteroaryl, —N(R^{6D1})(R^{6D2}), —C(O)N(R^{6D3})(R^{6D4}), or —N(R^{6D5})C(O)R^{6D6};
wherein the C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, or —C(O)-heteroaryl of D6 is unsubstituted or substituted with one or more substituents selected from the group consisting of halo, —CN, C₁₋₆alkyl, C₁₋₆ haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{6D10})₂, wherein each R^{6D10} is independently H or C₁₋₆alkyl;
R^{6D1}, R^{6D3}, and R^{6D5} are independently H or C₁₋₆alkyl;
R^{6D2} is aryl or heteroaryl, wherein the aryl or heteroaryl of R^{6D2} is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{6D11})₂, wherein each R^{6D11} is independently H or C₁₋₆alkyl; and
R^{6D4} and R^{6D6} are independently C₁₋₆alkyl, cycloalkyl, heterocyclyl, aryl, or heteroaryl, wherein each C₁₋₆alkyl, cycloalkyl, heterocyclyl, aryl, or heteroaryl of R^{6D4} and R^{6D6} is unsubstituted or substituted with one or more substituents independently selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{6D12})₂, wherein each R^{6D12} is independently H or C₁₋₆alkyl.

Embodiment I-7. The compound of embodiment I-2, and stereoisomers and pharmaceutically acceptable salts thereof, wherein the compound is of Formula VII:

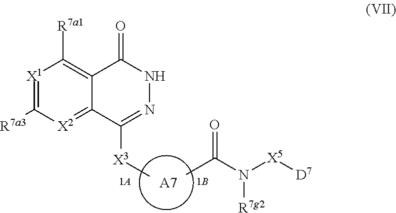

X¹ is —N— or —CR^{7a2}—;
X² is —N— or —CR^{7a4}—;
R^{7a1}, R^{7a2}, R^{7a3}, and R^{7a4} are independently H, halo, —OH, C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, —SO₂-cycloalkyl, —NR^{7L1}R^{7L2}, or C₂₋₆alkynyl-NR^{7L3}R^{7L4};
wherein if X¹ is —CR^{7a2}— and X² is —CR^{7a4}—, then at least one of R^{7a1}, R^{7a2}, R^{7a3}, and R^{7a4} is not H;
wherein each C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, or —SO₂-cycloalkyl of R^{7a1}, R^{7a2}, R^{7a3}, and R^{7a4} is unsubstituted or substituted with one or more substituents independently selected from the group consisting of halo, —OH, —CN, C₁₋₆alkyl, C₁₋₆haloalkyl, cycloalkyl, halocycloalkyl, —O—C₁₋₆alkyl, and —C(O)NH₂;
R^{7L1}, R^{7L2}, R^{7L3}, and R^{7L4} are independently H, C₁₋₆alkyl, C₁₋₆alkynyl, or cycloalkyl, wherein each C₁₋₆alkyl, C₂₋₆alkynyl, or cycloalkyl of R^{7L1}, R^{7L2}, R^{7L3}, and R^{7L4} is unsubstituted or substituted with one or more substituents independently selected from the group consisting of halo, C₁₋₆alkyl, C₁₋₆haloalkyl, cycloalkyl, and halocycloalkyl;
X³ is —O—, —NR^{7b1}—, or —CR^{7b2}R^{7b3}—, wherein R^{7b1}, R^{7b2}, and R^{7b3} are independently H or C₁₋₆alkyl;
A7 is

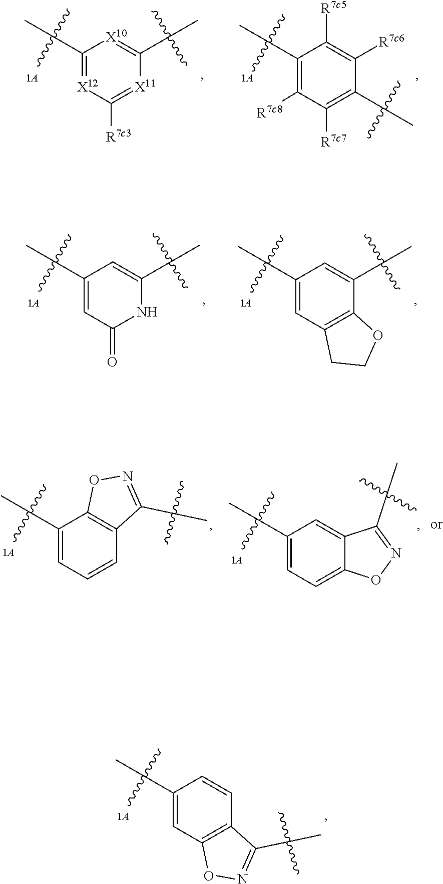

wherein a bond marked 1A is to X³;
X¹⁰ is —N— or —CR^{7c1}—, X¹¹ is —N— or —CR^{7c2}—, and X¹² is —N— or —CR^{7c4}—;
R^{7c1}, R^{7c2}, R^{7c3}, R^{7c4}, R^{7c5}, R^{7c6}, R^{7c7}, and R^{7c8} are independently H, halo, —CN, —SO₂CH₃, —SO₂NH₂, or —NHSO₂CH₃;
X⁵ is —CH(R^{7g3})— or —CH(R^{7g4})CH₂N(R^{7g5})—;
R^{7g2}, R^{7g3}, R^{7g4}, and R^{7g5} are independently H or C₁₋₆alkyl;
D7 is C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, —C(O)-heteroaryl, —N(R^{7D1})(R^{7D2}), —C(O)N(R^{7D3})(R^{7D4}), or —N(R^{7D5})C(O)R^{7D6};
wherein the C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, or —C(O)-heteroaryl of D7 is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{7D10}), wherein each R^{7D10} is independently H or C₁₋₆alkyl;
R^{7D1}, R^{7D3}, and R^{7D5} are independently H or C₁₋₆alkyl;
R^{7D2} is aryl or heteroaryl, wherein the aryl or heteroaryl of R^{7D2} is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{7D11)}₂, wherein each R^{7D11} is independently H or C₁₋₆alkyl; and
R^{7D4} and R^{7D6} are independently C₁₋₆alkyl, cycloalkyl, heterocyclyl, aryl, or heteroaryl, wherein each C₁₋₆alkyl, cycloalkyl, heterocyclyl, aryl, or heteroaryl of R^{7D4} and R^{7D6} is unsubstituted or substituted with one or more substituents independently selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆ haloalkyl, —C(O)-cycloalkyl, and —C(O)N(R^{7D12})₂, wherein each R^{7D12} is independently H or C₁₋₆alkyl.

Embodiment I-8. The compound of any one of embodiments I-1 to I-7, and stereoisomers and pharmaceutically acceptable salts thereof, wherein X¹ is —CR^{1a2}—, —CR^{2a2}—, —CR^{3a2}—, —CR^{4a2}—, —CR^{5a2}—, —CR^{6a2}—, or —CR^{7a2}—.

Embodiment I-9. The compound of any one of embodiments I-1 to I-8, and stereoisomers and pharmaceutically acceptable salts thereof, wherein X² is —CR^{1a4}—. —CR^{2a4}—, —CR^{3a4}—, —CR^{4a4}—, —CR^{5a4}—, —CR^{6a4}—, or —CR^{7a4}—.

Embodiment I-10. The compound of any one of embodiments I-1, I-2, I-4, I-8, and I-9, and stereoisomers and pharmaceutically acceptable salts thereof, wherein R^{1a2}, R^{2a2}, or R^{4a2} is H, Cl, Br, I, —OH, C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, —SO₂-cycloalkyl, —NR^{1L1}R^{1L2}, C₂₋₆alkynyl-NR^{1L3}R^{1L4}, —NR^{2L1}R^{2L2}, C₂₋₆alkynyl-NR^{2L3}R^{2L4}, —NR^{4L1}R^{4L2}, or C₂₋₆alkynyl-NR^{4L3}R^{4L4};
wherein each C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, or —SO₂-cycloalkyl of R^{1a2}, R^{2a2}, or R^{4a2} is unsubstituted or substituted with one or more substituents independently selected from the group consisting of halo, —OH, —CN, C₁₋₆alkyl, C₁₋₆haloalkyl, cycloalkyl, halocycloalkyl, —O—C₁₋₆alkyl, and —C(O)NH₂.

Embodiment I-11. The compound of any one of embodiments I-1, I-2, I-4, and I-8 to I-10, and stereoisomers and pharmaceutically acceptable salts thereof, wherein R^{1a4}, R^{2a4}, or R^{4a4} is H, F, Br, I, —OH, C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, —SO₂-cycloalkyl, —NR^{1L1}R^{1L2}, C₂₋₆ alkynyl-NR^{1L3}R^{1L4}, —NR^{2L1}R^{2L2}, C₂₋₆alkynyl-NR^{2L3}R^{2L4}, —NR^{4L1}R^{4L2}, or C₂₋₆alkynyl-NR^{4L3}R^{4L4};
wherein each C₁₋₆alkyl, C₂₋₆alkenyl, C₂₋₆alkynyl, cycloalkyl, heterocyclyl, aryl, heteroaryl, —O—C₁₋₆alkyl, —O—C₁₋₆alkenyl, —O—C₂₋₆alkynyl, —O-cycloalkyl, —O-heterocyclyl, —O-aryl, —O-heteroaryl, or —SO₂-cycloalkyl of R^{1a4}, R^{2a4}, or R^{4a4} is unsubstituted or substituted with one or more substituents independently selected from the group consisting of halo, —OH, —CN, C₁₋₆alkyl, C₁₋₆haloalkyl, cycloalkyl, halocycloalkyl, —O—C₁₋₆alkyl, and —C(O)NH₂.

Embodiment I-12. The compound of any one of embodiments I-1 to I-11, and stereoisomers and pharmaceutically acceptable salts thereof, wherein one, two, three, or four of R^{1a1}, R^{1a2}, R^{1a3}, and R^{1a4}, R^{2a1}, R^{2a2}, R^{2a3}, and R^{2a4}, R^{3a1}, R^{3a2}, R^{3a3}, and R^{3a4}, R^{4a1}, R^{4a2}, R^{4a3} and R^{4a4}, R^{5a1}, R^{5a2}, R^{5a3}, and R^{5a4}, R^{6a1}, R^{6a2}, R^{6a3}, and R^{6a4}, or R^{7a1}, R^{7a2}, R^{7a3}, and R^{7a4} is not H.

Embodiment I-13. The compound of any one of embodiments I-1 to I-12, and stereoisomers and pharmaceutically acceptable salts thereof, wherein R^{1a3}, R^{2a3}, R^{3a3}, R^{4a3}, R^{5a3}, R^{6a3}, or R^{7a3} is not H.

Embodiment I-14. The compound of any one of embodiments I-1 to I-13, and stereoisomers and pharmaceutically acceptable salts thereof, wherein one, two, three, or four of R^{1a1}, R^{1a2}, R^{1a3}, and R^{1a4}, R^{2a1}, R^{2a2}, R^{2a3}, and R^{2a4}, R^{3a1}, R^{3a2}, R^{3a3}, and R^{3a4}, R^{4a1}, R^{4a2}, R^{4a3}, and R^{4a4}, R^{5a1}, R^{5a2}, R^{5a3}, and R^{5a4}, R^{6a1}, R^{6a2}, R^{6a3}, and R^{6a4}, or R^{7a1}, R^{7a1}, R^{7a2}, and R^{7a4} is F or Cl.

Embodiment I-15. The compound of any one of embodiments I-1 to I-14, and stereoisomers and pharmaceutically acceptable salts thereof, wherein R^{1a1}, R^{2a1}, R^{3a1}, R^{4a1}, R^{5a1}, R^{6a1}, or R^{7a1} is F or Cl.

Embodiment I-16. The compound of any one of embodiments I-1 to I-9 and I-11 to I-15, and stereoisomers and pharmaceutically acceptable salts thereof, wherein R^{1a1}, R^{2a2}, R^{3a2}, R^{4a2}, R^{5a2}, R^{6a2}, or R^{7a2} is F or Cl.

Embodiment I-17. The compound of any one of embodiments I-1 to I-16, and stereoisomers and pharmaceutically acceptable salts thereof, wherein R^{1a3}, R^{2a3}, R^{3a3}, R^{4a3}, R^{5a3}, R^{6a3}, or R^{7a3} is F or Cl.

Embodiment I-18. The compound of any one of embodiments I-1 to I-17, and stereoisomers and pharmaceutically acceptable salts thereof, wherein one, two, three, or four of R^{1a1}, R^{1a2}, R^{1a3}, and R^{1a4}, R^{2a1}, R^{2a2}, R^{2a3}, and R^{2a4}, R^{3a1}, R^{3a2}, R^{3a3}, and R^{3a4}, R^{4a1}, R^{4a2}, R^{4a3}, and R^{4a4}, R^{5a1}, R^{5a2}, R^{5a3}, and R^{5a4}, R^{6a1}, R^{6a2}, R^{6a3}, and R^{6a4}, or R^{7a1}, R^{7a2}, R^{7a3}, and R^{7a4} is —OH.

Embodiment I-19. The compound of any one of embodiments I-1 to I-16 and I-18, and stereoisomers and pharmaceutically acceptable salts thereof, wherein R^{1a3}, R^{2a3}, R^{3a3}, R^{4a3}, R^{5a3}, R^{6a3}, or R^{7a3} is —OH.

Embodiment I-20. The compound of any one of embodiments I-1 to I-19, and stereoisomers and pharmaceutically acceptable salts thereof, wherein one, two, three, or four of R^{1a1}, R^{1a2}, R^{1a3}, and R^{1a4}, R^{2a1}, R^{2a2}, R^{2a3}, and R^{2a4}, R^{3a1}, R^{3a2}, R^{3a3}, and R^{3a4}, R^{4a1}, R^{4a2}, R^{4a3}, and R^{4a4}, R^{5a1}, R^{5a2}, R^{5a3}, and R^{5a4}, R^{6a1}, R^{6a2}, R^{6a3}, and R^{6a4}, or R^{7a1}, R^{7a2}, R^{7a3}, and R^{7a4} is —O—C₁₋₆alkyl or —O-cycloalkyl, wherein the —O—C₁₋₆alkyl or —O-cycloalkyl is unsubstituted or substituted with halo or —CN.

Embodiment I-21. The compound of any one of embodiments I-1 to I-16, I-18, and I-20, and stereoisomers and pharmaceutically acceptable salts thereof, wherein R^{1a3}, R^{2a3}, R^{3a3}, R^{4a3}, R^{5a3}, R^{6a3}, or R^{7a3} is —O—C₁₋₆alkyl or —O-cycloalkyl, wherein the —O—C₁₋₆alkyl or —O-cycloalkyl is unsubstituted or substituted with halo or —CN.

Embodiment I-22. The compound of any one of embodiments I-1 to I-21, and stereoisomers and pharmaceutically acceptable salts thereof, wherein one, two, three, or four of R^{1a1}, R^{1a2}, R^{1a3}, and R^{1a4}, R^{2a1}, R^{2a2}, R^{2a3}, and R^{2a4}, R^{3a1}, R^{3a2}, R^{3a3}, and R^{3a4}, R^{4a1}, R^{4a2}, R^{4a3}, and R^{4a4}, R^{5a1}, R^{5a2}, R^{5a3}, and R^{5a4}, R^{6a1}, R^{6a2}, R^{6a3}, and R^{6a4}, or R^{7a1}, R^{7a2}, R^{7a3}, and R^{7a4} is —O—C₂₋₆-alkynyl, wherein the —O—C₂₋₆alkynyl is unsubstituted or substituted with halo.

Embodiment I-23. The compound of any one of embodiments I-1 to I-16, I-18, I-20, and I-22, and stereoisomers and pharmaceutically acceptable salts thereof, wherein R^{1a3}, R^{2a3}, R^{3a3}, R^{4a3}, R^{5a3}, R^{6a3}, or R^{7a3} is —O—C₂₋₆alkynyl, wherein the —O—C₂₋₆alkynyl is unsubstituted or substituted with halo.

Embodiment I-24. The compound of any one of embodiments I-1 to I-23, and stereoisomers and pharmaceutically acceptable salts thereof, wherein one, two, three, or four of R^{1a1}, R^{1a2}, R^{1a3}, and R^{1a4}, R^{2a1}, R^{2a2}, R^{2a3}, and R^{2a4}, R^{3a1}, R^{3a2}, R^{3a3}, and R^{3a4}, R^{4a1}, R^{4a2}, R^{4a3}, and R^{4a4}, R^{5a1}, R^{5a2}, R^{5a3}, and R^{5a4}, R^{6a1}, R^{6a2}, R^{6a3}, and R^{6a4}, or R^{7a1}, R^{7a2}, R^{7a3}, and R^{7a4} is —O-aryl or —O-heteroaryl, wherein the —O-aryl or —O-heteroaryl is unsubstituted or substituted with halo.

Embodiment I-25. The compound of any one of embodiments I-1 to I-16, I-18, I-20, I-22, and I-24, and stereoisomers and pharmaceutically acceptable salts thereof, wherein R^{1a3}, R^{2a3}, R^{3a3}, R^{4a3}, R^{5a3}, R^{6a3}, or R^{7a3} is —O-aryl or —O-heteroaryl, wherein the —O-aryl or —O-heteroaryl is unsubstituted or substituted with halo.

Embodiment I-26. The compound of any one of embodiments I-1 to I-25, and stereoisomers and pharmaceutically acceptable salts thereof, wherein two of R^{1a1}, R^{1a2}, R^{1a3}, and R^{1a4}, R^{2a1}, R^{2a2}, R^{2a3}, and R^{2a4}, R^{3a1}, R^{3a2}, R^{3a3}, and R^{3a4}, R^{4a1}, R^{4a2}, R^{4a3}, and R^{4a4}, R^{5a1}, R^{5a2}, R^{5a3}, and R^{5a4}, R^{6a1}, R^{6a2}, R^{6a3}, and R^{6a4}, or R^{7a1}, R^{7a2}, R^{7a3}, and R^{7a4} is not H.

Embodiment I-27. The compound of any one of embodiments I-1 to I-26, and stereoisomers and pharmaceutically acceptable salts thereof, wherein R^{1a1}, R^{2a1}, R^{3a1}, R^{4a1}, R^{5a1}, R^{6a1}, or R^{7a1} is F or Cl and R^{1a2}, R^{2a2}, R^{3a2}, R^{4a2}, R^{5a2}, R^{6a2}, or R^{7a2} is F or Cl.

Embodiment I-28. The compound of any one of embodiments I-1 to I-16, I-18, I-20 to I-22, I-24, and I-26 and stereoisomers and pharmaceutically acceptable salts thereof, wherein R^{1a2}, R^{2a2}, R^{3a2}, R^{4a2}, R^{5a2}, R^{6a2}, or R^{7a2} is F or Cl and R^{1a3}, R^{2a3}, R^{3a3}, R^{4a3}, R^{5a3}, R^{6a3}, or R^{7a3} is —O—C₁₋₆alkyl or —O-cycloalkyl, wherein the —O—C₁₋₆-alkyl or —O-cycloalkyl is unsubstituted or substituted with halo.

Embodiment I-29. The compound of any one of embodiments I-1 to I-28, and stereoisomers and pharmaceutically acceptable salts thereof, wherein X³ is —O—.

Embodiment I-30. The compound of any one of embodiments I-1 to I-29, and stereoisomers and pharmaceutically acceptable salts thereof, wherein X³ is —CH₂—.

Embodiment I-31. The compound of any one of embodiments I-1, 1-3, and I-8 to I-30, and stereoisomers and pharmaceutically acceptable salts thereof, wherein X⁴ is —O—.

Embodiment I-32. The compound of any one of embodiments I-1, I-3, and I-8 to I-30, and stereoisomers and pharmaceutically acceptable salts thereof, wherein X⁴ is —NR^{3f2}—.

Embodiment I-33. The compound of any one of embodiments I-1, I-3, and I-8 to I-30, and stereoisomers and pharmaceutically acceptable salts thereof, wherein X⁴ is —CR^{3f3}R^{3f4}—.

Embodiment I-34. The compound of any one of embodiments I-1, I-2 and I-4 to I-30, and stereoisomers and pharmaceutically acceptable salts thereof, wherein A1 A2, A4, A5, A6, or A7 is

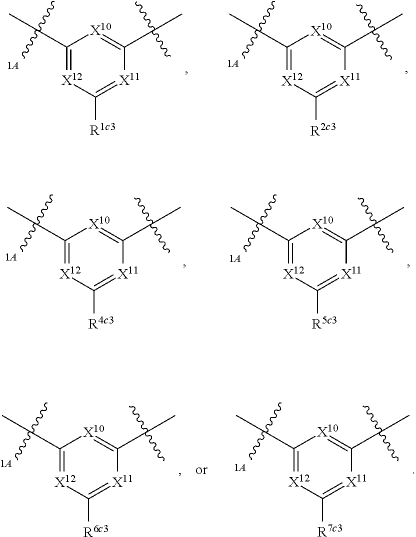

Embodiment I-35. The compound of any one of embodiments I-1, I-2, and I-4 to I-34, and stereoisomers and pharmaceutically acceptable salts thereof, wherein one or more of X¹⁰, X¹¹, and X¹² is —N—.

Embodiment I-36. The compound of any one of embodiments I-1, I-2, I-4 to I-30, and I-34, and stereoisomers and pharmaceutically acceptable salts thereof, wherein X¹⁰ is —CR^{1c1}—, —CR^{2c1}—, —CR^{4c1}—, —CR^{5c1}—, —CR^{6c1}—, or —CR^{7c1}—, X¹¹ is —CR^{1c2}—, —CR^{2c2}—, —CR^{4c2}—, —CR^{5c2}—, —CR^{6c2}—, or —CR^{7c2}—, and X¹² is —CR^{1c3}—, —CR^{2c3}—, —CR^{4c3}—, —CR^{5c3}—, —CR^{6c3}—, or —CR^{7c3}—.

Embodiment I-37. The compound of any one of embodiments I-1, I-2, I-4 to I-30, and I-34 to I-36, and stereoisomers and pharmaceutically acceptable salts thereof, wherein one, two, three, or four of R^{1c1}, R^{1c2}, R^{1c3}, and R^{1c4}, R^{2c1}, R^{2c2}, R^{2c3}, and R^{2c4}, R^{4c1}, R^{4c2}, R^{4c3}, and R^{4c4}, R^{5c1}, R^{5c2}, R^{5c3}, and R^{5c4}, R^{6c1}, R^{6c2}, R^{6c3}, and R^{6c4}, or R^{7c1}, R^{7c2}, R^{7c3}, and R^{7c4} is not H.

Embodiment I-38. The compound of any one of embodiments I-1, I-2, I-4 to I-30, and I-34 to I-37 and stereoisomers and pharmaceutically acceptable salts thereof, wherein R^{1c2}, R^{2c2}, R^{4c2}, R^{5c2}, R^{6c2}, or R^{7c2} is F and R^{1c1}, R^{1c3}, and R^{1c4}, R^{2c1}, R^{2c3}, and R^{2c4}, R^{4c1}, R^{4c3}, and R^{4c4}, R^{5c1}, R^{5c3}, and R^{5c4}, R^{6c1}, R^{6c3}, and R^{6c4}, or R^{7c1}, R^{7c3}, and R^{7c4} are H.

Embodiment I-39. The compound of any one of embodiments I-1, I-2, I-4 to I-30, and I-34 to I-36, and stereoisomers and pharmaceutically acceptable salts thereof, wherein R^{1c1}, R^{1c2}, R^{1c3}, and R^{1c4}, R^{2c1}, R^{2c2}, R^{2c3}, and R^{2c4}, R^{4c1}, R^{4c2}, R^{4c3}, and R^{4c4}, R^{5c1}, R^{5c2}, R^{5c3}, and R^{5c4}, R^{6c1}, R^{6c2}, R^{6c3}, and R^{6c4}, or R^{7c1}, R^{7c2}, R^{7c3} and R^{7c4} are H.

Embodiment 140. The compound of any one of embodiments I-1, I-2, and I-4 to I-30, and stereoisomers and pharmaceutically acceptable salts thereof, wherein A1, A2, A4, A5, A6, or A7 is

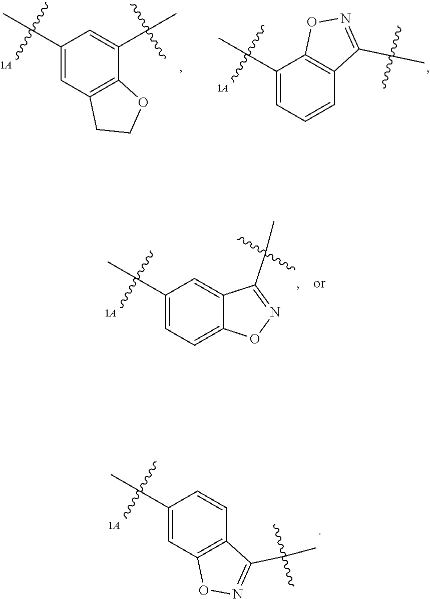 [SMILES: *c1cc(*)c2c(c1)CCO2 ; *c1noc2c(*)cccc12 ; *c1ccc2onc(*)c2c1 ; *c1ccc2c(*)noc2c1]

Embodiment I-41. The compound of any one of embodiments I-1 to I-8 to I-40, and stereoisomers and pharmaceutically acceptable salts thereof, wherein B3, B4, B5, or B6 is a 3-membered monocyclic heterocyclediyl, a 4-membered monocyclic heterocyclediyl, a 5-membered monocyclic heterocyclediyl comprising 2 or more N, a 6-membered monocyclic heterocyclediyl comprising 2 or more N, a 7-membered monocyclic heterocyclediyl, an 8-membered monocyclic heterocyclediyl, a 7 to 18-membered polycyclic heterocyclediyl, or a 7 to 18-membered spirocyclic heterocyclediyl;
wherein the 3-membered monocyclic heterocyclediyl, 4-membered monocyclic heterocyclediyl, 5-membered monocyclic heterocyclediyl, 6-membered monocyclic heterocyclediyl, 7-membered monocyclic heterocyclediyl, 8-membered monocyclic heterocyclediyl, 7 to 18-membered polycyclic heterocyclediyl, or 7 to 18-membered spirocyclic heterocyclediyl of B3, B4, B5, or B6 is unsubstituted or substituted with one or more substituents independently selected from the group consisting of halo, C₁₋₆ alkyl, and oxo.

Embodiment I-42. The compound of any one of embodiments I-1 to I-6 and I-8 to I-40, and stereoisomers and pharmaceutically acceptable salts thereof, wherein B3, B4, B5, or B6 is a 3 to 8-membered monocyclic heterocyclediyl, wherein the 3 to 8-membered monocyclic heterocyclediyl is unsubstituted or substituted with one or more substituents selected from the group consisting of halo, C₁₋₆ alkyl, and oxo.

Embodiment I-43. The compound of any one of embodiments I-1 to I-6 and I-8 to I-42, and stereoisomers and pharmaceutically acceptable salts thereof, wherein B3, B4, B5, or B6 is a 4-membered monocyclic heterocyclediyl, wherein the 4-membered monocyclic heterocyclediyl is unsubstituted or substituted with one or more substituents selected from the group consisting of halo, C₁₋₆alkyl, and oxo.

Embodiment I-44. The compound of any one of embodiments I-1 to I-6 and I-8 to I-43, and stereoisomers and pharmaceutically acceptable salts thereof, wherein B3, B4, B5, or B6 is

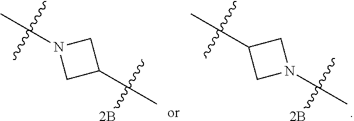

Embodiment I-45. The compound of any one of embodiments I-1 to I-6, 1-8 to I-40 and I-42, and stereoisomers and pharmaceutically acceptable salts thereof, wherein B3, B4, B5, or B6 is a 6-membered monocyclic heterocyclediyl, wherein the 6-membered monocyclic heterocyclediyl is unsubstituted or substituted with one or more substituents selected from the group consisting of halo, C₁₋₆alkyl, and oxo.

Embodiment I-46. The compound of any one of embodiments I-1 to I-6, I-8 to I-42 and I-45, and stereoisomers and pharmaceutically acceptable salts thereof, wherein B3, B4, B5, or B6 is

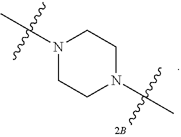 [SMILES: *N1CCN(*)CC1]

Embodiment I-47. The compound of any one of embodiments I-1 to I-6 and I-8 to I-42, and stereoisomers and pharmaceutically acceptable salts thereof, wherein B3, B4, B5, or B6 is

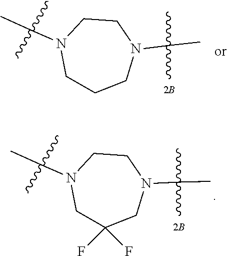 [SMILES: *N1CCCN(*)CC1 ; *N1CCN(*)CC(F)(F)C1]

Embodiment I-48. The compound of any one of embodiments I-1 to I-6 and I-8 to I-41, and stereoisomers and pharmaceutically acceptable salts thereof, wherein B3, B4, B5, or B6 is a 7 to 18-membered polycyclic heterocyclediyl, wherein the 7 to 18-membered polycyclic heterocyclediyl is unsubstituted or substituted with one or more substituents selected from the group consisting of halo, C₁₋₆ alkyl, and oxo.

Embodiment I-49. The compound of any one of embodiments I-1 to I-6, 1-8 to I-41, and I-48, and stereoisomers and pharmaceutically acceptable salts thereof, wherein B3, B4, B5, or B6 is

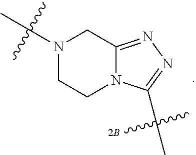 [SMILES: *c1nnc2n1CCN(*)C2]

Embodiment I-50. The compound of any one of embodiments I-1 to I-6, 1-8 to I-41, and I-48, and stereoisomers and pharmaceutically acceptable salts thereof, wherein B3, B4, B5, or B6 is B6 is

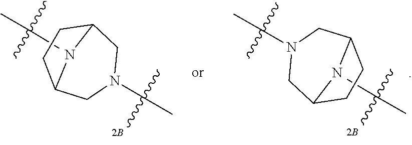

Embodiment I-51. The compound of any one of embodiments I-1 to I-6, 1-8 to I-41, and I-48, and stereoisomers and pharmaceutically acceptable salts thereof, wherein B3, B4, B5, or B6 is

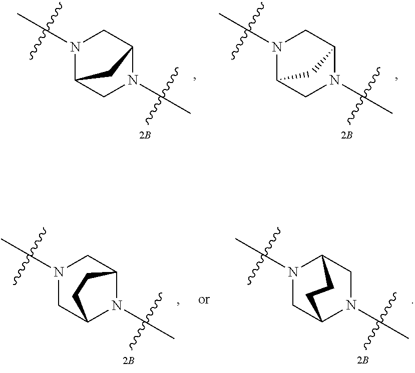

Embodiment I-52. The compound of any one of embodiments I-1 to I-6 and I-8 to I-41, and stereoisomers and pharmaceutically acceptable salts thereof, wherein B3, B4, B5, or B6 is a 7 to 18-membered spirocyclic heterocyclediyl, wherein the 7 to 18-membered spirocyclic heterocyclediyl is unsubstituted or substituted with one or more substituents selected from the group consisting of halo, C₁₋₆alkyl, and oxo.

Embodiment I-53. The compound of any one of embodiments I-1 to I-6 and I-8 to I-52, and stereoisomers and pharmaceutically acceptable salts thereof, wherein the monocyclic heterocyclediyl, polycyclic heterocyclediyl, or spirocyclic heterocyclediyl of B3, B4, B5, or B6 comprises one or more N.

Embodiment I-54. The compound of any one of embodiments I-1 to I-6, 1-8 to I-43, and I-45 to I-54, and stereoisomers and pharmaceutically acceptable salts thereof, wherein the monocyclic heterocyclediyl, polycyclic heterocyclediyl, or spirocyclic heterocyclediyl of B3, B4, B5, or B6 comprises two or more N.

Embodiment I-55. The compound of any one of embodiments I-1, I-5, 1-8 to I-30, and I-34 to I-54, and stereoisomers and pharmaceutically acceptable salts thereof, wherein R^{5g1} is H.

Embodiment I-56. The compound of any one of embodiments I-1, I-5, 1-8 to I-30, and I-34 to I-54, and stereoisomers and pharmaceutically acceptable salts thereof, wherein R^{5g1} is —CH₃.

Embodiment I-57. The compound of any one of embodiments I-1, I-7, I-8 to I-30, and I-34 to I-54, and stereoisomers and pharmaceutically acceptable salts thereof, wherein R^{7g2} is H.

Embodiment I-58. The compound of any one of embodiments I-1, I-7, I-8 to I-30, and I-34 to I-54, and stereoisomers and pharmaceutically acceptable salts thereof, wherein R^{7g2} is —CH₃.

Embodiment I-59. The compound of any one of embodiments I-1, I-7, I-8 to I-30, I-34 to I-54, I-57, and I-58 and stereoisomers and pharmaceutically acceptable salts thereof, wherein X⁵ is —CH(R^{7g3})—, wherein R^{7g3} is H or C₁₋₆alkyl.

Embodiment I-60. The compound of any one of embodiments I-1, I-7, I-8 to I-30, I-34 to I-54, I-57, and I-58, and stereoisomers and pharmaceutically acceptable salts thereof, wherein X⁵ is —CH(R^{7g4})CH₂N(R^{7g5})—, wherein R^{7g4} and R^{7g5} are independently H or C₁₋₆alkyl.

Embodiment I-61. The compound of any one of embodiments I-1 to I-60, and stereoisomers and pharmaceutically acceptable salts thereof, wherein D3, D4, D5, D6, or D7 is C₁₋₆alkyl, wherein the C₁₋₆ alkyl is unsubstituted or substituted with halo.

Embodiment I-62. The compound of any one of embodiments I-1 to I-60, and stereoisomers and pharmaceutically acceptable salts thereof, wherein D3, D4, D5, D6, or D7 is cycloalkyl, wherein the cycloalkyl is unsubstituted or substituted with halo.

Embodiment I-63. The compound of any one of embodiments I-1 to I-60, and stereoisomers and pharmaceutically acceptable salts thereof, wherein D3, D4, D5, D6, or D7 is aryl, wherein the aryl is unsubstituted or substituted with one or more substituents selected from the group consisting of halo, —CN, C₁₋₆alkyl, C₁₋₆haloalkyl, cycloalkyl, —OH, —C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, —C(O)NH₂, —C(O)NHCH₃, and —C(O)N(CH₃)₂.

Embodiment I-64. The compound of any one of embodiments I-1 to I-60 and I-63, and stereoisomers and pharmaceutically acceptable salts thereof, wherein D3, D4, D5, D6, or D7 is a monocyclic 6-membered aryl, wherein the monocyclic 6-membered aryl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, —C(O)NH₂, —C(O)NHCH₃, and —C(O)N(CH₃)₂.

Embodiment I-65. The compound of any one of embodiments I-1 to I-60, and stereoisomers and pharmaceutically acceptable salts thereof, wherein D3, D4, D5, D6, or D7 is heteroaryl, wherein the heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-C₁₋₆ alkyl, C₁₋₆alkyl-O—C₁₋₆haloalkyl, —C(O)-cycloalkyl, —C(O)NH₂, —C(O)NHCH₃, and —C(O)N(CH₃)₂.

Embodiment I-66. The compound of any one of embodiments I-1 to I-60 and I-65, and stereoisomers and pharmaceutically acceptable salts thereof, wherein D3, D4, D5, D6, or D7 is a monocyclic 5 or 6-membered heteroaryl comprising one or more N, wherein the monocyclic 5 or 6-membered heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, —C(O)NH₂, —C(O)NHCH₃, and —C(O)N(CH₃)₂.

Embodiment I-67. The compound of any one of embodiments I-1 to I-60, and stereoisomers and pharmaceutically acceptable salts thereof, wherein D3, D4, D5, D6, or D7 is —O—C₁₋₆alkyl or C₁₋₆alkyl-O—C₁₋₆alkyl, wherein the —O—C₁₋₆alkyl or C₁₋₆alkyl-O—C₁₋₆alkyl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, —C(O)NH₂, —C(O)NHCH₃, and —C(O)N(CH₃)₂.

Embodiment I-68. The compound of any one of embodiments I-1 to I-60, and stereoisomers and pharmaceutically acceptable salts thereof, wherein D3, D4, D5, D6, or D7 is —O-aryl or —O-heteroaryl, wherein the —O-aryl or —O-heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, —C(O)NH₂, —C(O)NHCH₃, and —C(O)N(CH₃)₂.

Embodiment I-69. The compound of any one of embodiments I-1 to I-60, and stereoisomers and pharmaceutically acceptable salts thereof, wherein D3, D4, D5, D6, or D7 is —C(O)—C₁₋₆alkyl, —C(O)-cycloalkyl, or —C(O)-heterocyclyl, wherein the —C(O)—C₁₋₆alkyl, —C(O)-cycloalkyl, or —C(O)-heterocyclyl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, —C(O)NH₂, —C(O)NHCH₃, and —C(O)N(CH₃)₂.

Embodiment I-70. The compound of any one of embodiments I-1 to I-60, and stereoisomers and pharmaceutically acceptable salts thereof, wherein D3, D4, D5, D6, or D7 is —C(O)-aryl or —C(O)-heteroaryl, wherein the —C(O)-aryl or —C(O)-heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, —C(O)NH₂, —C(O)NHCH₃, and —C(O)N(CH₃)₂.

Embodiment I-71. The compound of any one of embodiments I-1 to I-60, and stereoisomers and pharmaceutically acceptable salts thereof, wherein D3, D4, D5, D6, or D7 is —N(R^{1D1})(R^{1D2}), —N(R^{2D1})(R^{2D2}), —N(R^{3D1})(R^{3D2}), —N(R^{4D1})(R^{4D2}), —N(R^{5D1})(R^{5D2}), —N(R^{6D1})(R^{6D2}), or —N(R^{7D1})(R^{7D2}).

Embodiment I-72. The compound of any one of embodiments I-1 to I-60, and stereoisomers and pharmaceutically acceptable salts thereof, wherein D3, D4, D5, D6, or D7 is —C(O)N(R^{1D3})(R^{1D4}), —C(O)N(R^{2D3})(R^{2D4}), —C(O)N(R^{3D3})(R^{3D4}), —C(O)N(R^{4D3})(R^{4D4}), —C(O)N(R^{5D3})(R^{5D4}), —C(O)N(R^{6D3})(R^{6D4}), or —C(O)N(R^{7D3})(R^{7D4}).

Embodiment I-73. The compound of any one of embodiments I-1 to I-60, and stereoisomers and pharmaceutically acceptable salts thereof, wherein D3, D4, D5, D6, or D7 is —N(R^{1D5})C(O)R^{1D6}—, —N(R^{2D5})C(O)R^{2D6}—, —N(R^{3D5})C(O)R^{3D6}—, —N(R^{4D5})C(O)R^{4D6}, —N(R^{5D5})C(O)R^{5D6}—, —N(R^{6D5})C(O)R^{6D6}—, or —N(R^{7D5})C(O)R^{7D6}—.

Embodiment I-74. A compound, and stereoisomers and pharmaceutically acceptable salts thereof, selected from the group consisting of

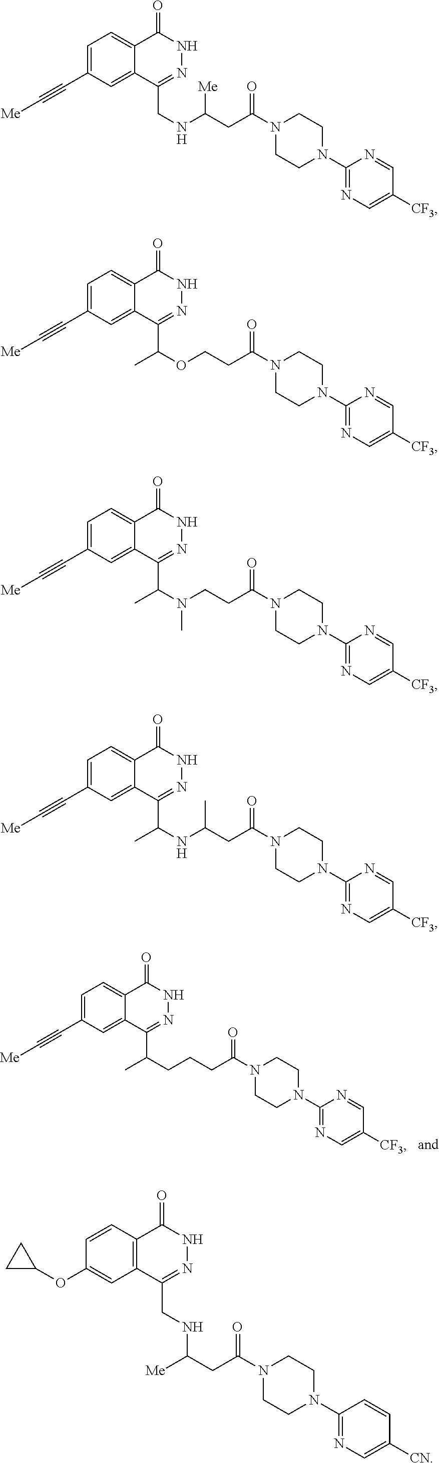 [SMILES: CC#Cc1ccc2c(=O)[nH]nc(CNC(C)CC(=O)N3CCN(c4ncc(C(F)(F)F)cn4)CC3)c2c1 ; CC#Cc1ccc2c(=O)[nH]nc(C(C)OCCC(=O)N3CCN(c4ncc(C(F)(F)F)cn4)CC3)c2c1 ; CC#Cc1ccc2c(=O)[nH]nc(C(C)N(C)CCC(=O)N3CCN(c4ncc(C(F)(F)F)cn4)CC3)c2c1 ; CC#Cc1ccc2c(=O)[nH]nc(C(C)NC(C)CC(=O)N3CCN(c4ncc(C(F)(F)F)cn4)CC3)c2c1 ; CC#Cc1ccc2c(=O)[nH]nc(C(C)CCCC(=O)N3CCN(c4ncc(C(F)(F)F)cn4)CC3)c2c1 ; CC(CC(=O)N1CCN(c2ccc(C#N)cn2)CC1)NCc1n[nH]c(=O)c2ccc(OC3CC3)cc12]

Embodiment I-75. A compound, and stereoisomers and pharmaceutically acceptable salts thereof, selected from the group consisting of

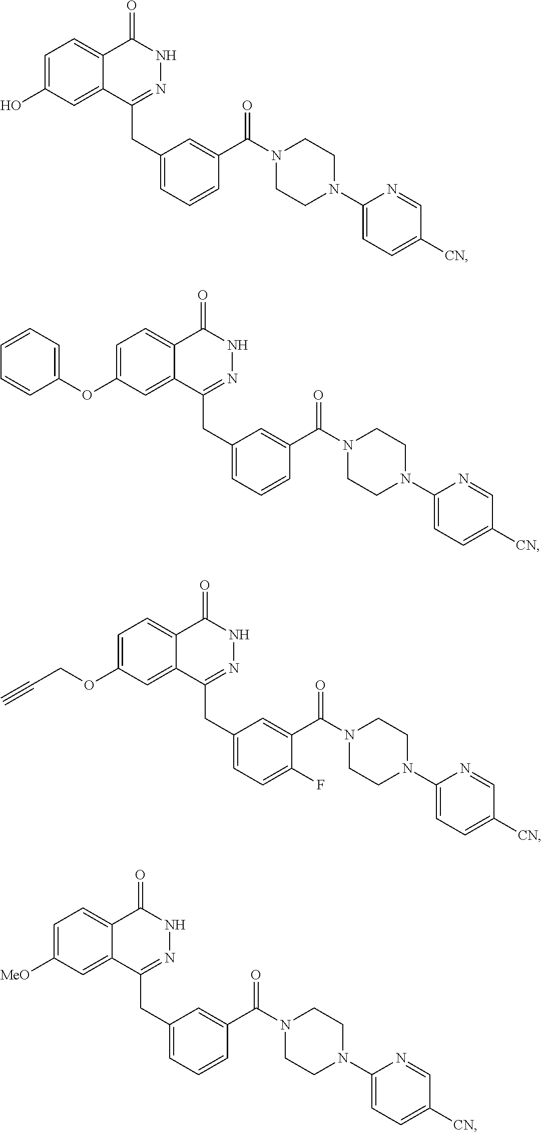 [SMILES: N#Cc1ccc(N2CCN(C(=O)c3cccc(Cc4n[nH]c(=O)c5ccc(O)cc45)c3)CC2)nc1 ; N#Cc1ccc(N2CCN(C(=O)c3cccc(Cc4n[nH]c(=O)c5ccc(Oc6ccccc6)cc45)c3)CC2)nc1 ; C#CCOc1ccc2c(=O)[nH]nc(Cc3ccc(F)c(C(=O)N4CCN(c5ccc(C#N)cn5)CC4)c3)c2c1 ; COc1ccc2c(=O)[nH]nc(Cc3cccc(C(=O)N4CCN(c5ccc(C#N)cn5)CC4)c3)c2c1]

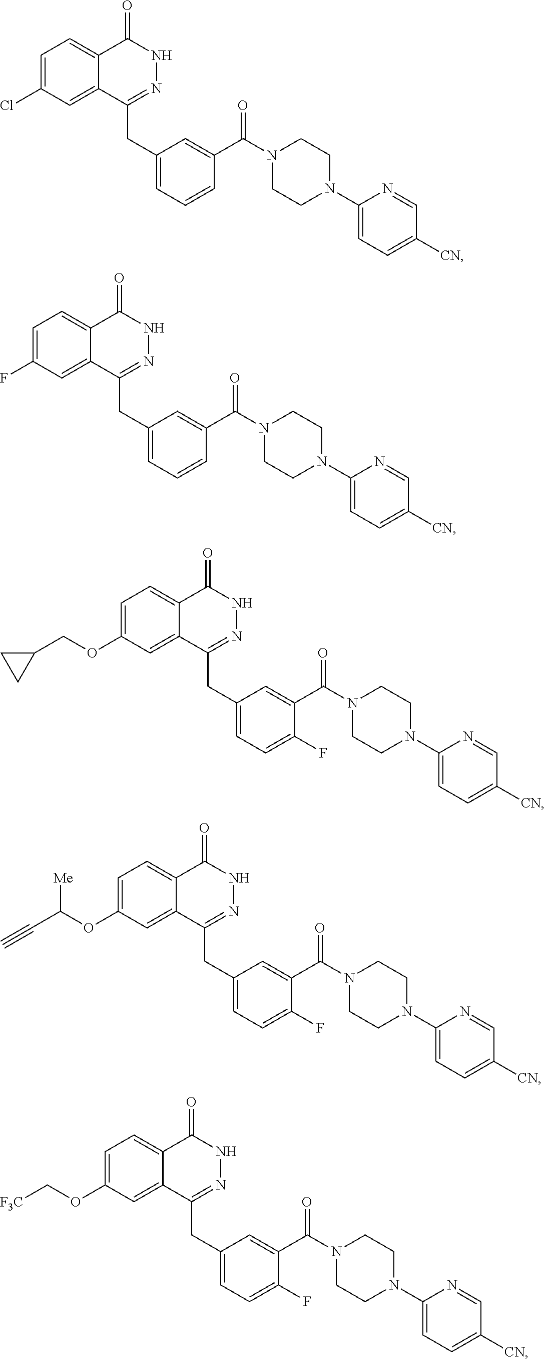 [SMILES: N#Cc1ccc(N2CCN(C(=O)c3cccc(Cc4n[nH]c(=O)c5ccc(Cl)cc45)c3)CC2)nc1 ; N#Cc1ccc(N2CCN(C(=O)c3cccc(Cc4n[nH]c(=O)c5ccc(F)cc45)c3)CC2)nc1 ; N#Cc1ccc(N2CCN(C(=O)c3cc(Cc4n[nH]c(=O)c5ccc(OCC6CC6)cc45)ccc3F)CC2)nc1 ; C#CC(C)Oc1ccc2c(=O)[nH]nc(Cc3ccc(F)c(C(=O)N4CCN(c5ccc(C#N)cn5)CC4)c3)c2c1 ; N#Cc1ccc(N2CCN(C(=O)c3cc(Cc4n[nH]c(=O)c5ccc(OCC(F)(F)F)cc45)ccc3F)CC2)nc1]

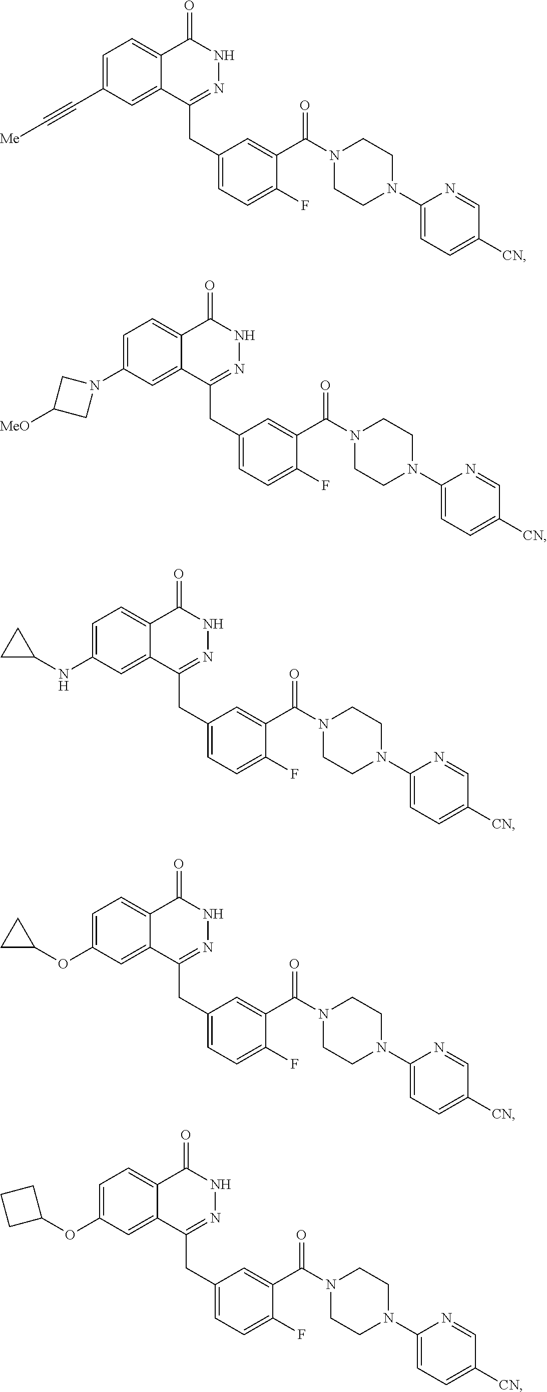 [SMILES: CC#Cc1ccc2c(=O)[nH]nc(Cc3ccc(F)c(C(=O)N4CCN(c5ccc(C#N)cn5)CC4)c3)c2c1 ; COC1CN(c2ccc3c(=O)[nH]nc(Cc4ccc(F)c(C(=O)N5CCN(c6ccc(C#N)cn6)CC5)c4)c3c2)C1 ; N#Cc1ccc(N2CCN(C(=O)c3cc(Cc4n[nH]c(=O)c5ccc(NC6CC6)cc45)ccc3F)CC2)nc1 ; N#Cc1ccc(N2CCN(C(=O)c3cc(Cc4n[nH]c(=O)c5ccc(OC6CC6)cc45)ccc3F)CC2)nc1 ; N#Cc1ccc(N2CCN(C(=O)c3cc(Cc4n[nH]c(=O)c5ccc(OC6CCC6)cc45)ccc3F)CC2)nc1]

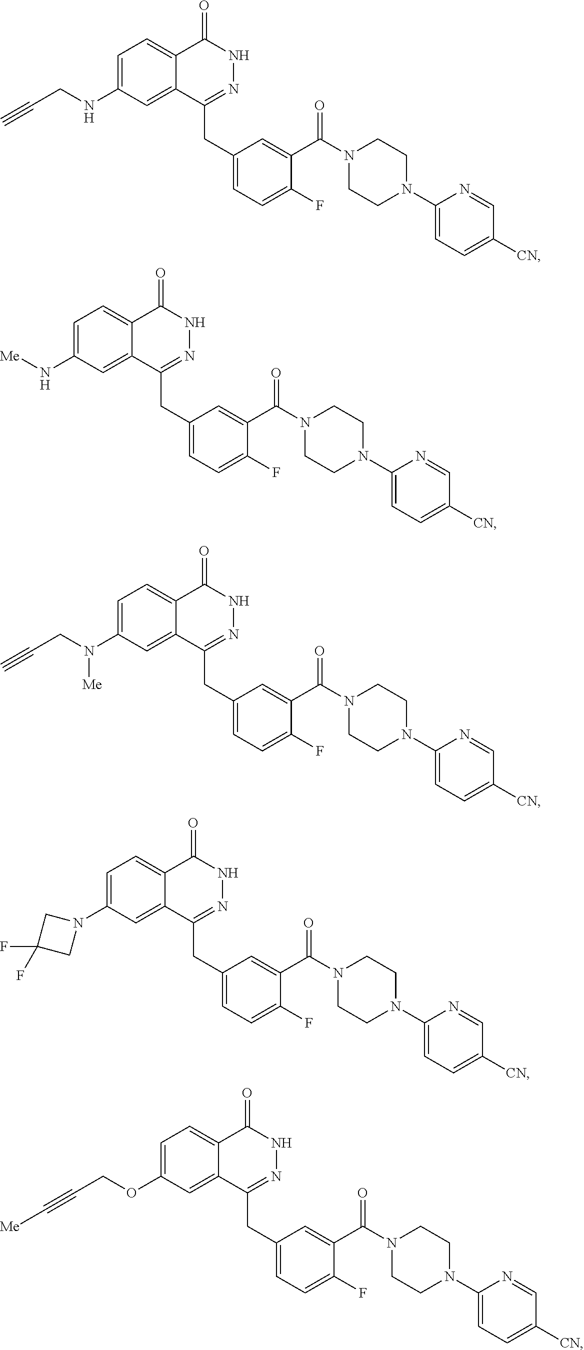 [SMILES: C#CCNc1ccc2c(=O)[nH]nc(Cc3ccc(F)c(C(=O)N4CCN(c5ccc(C#N)cn5)CC4)c3)c2c1 ; CNc1ccc2c(=O)[nH]nc(Cc3ccc(F)c(C(=O)N4CCN(c5ccc(C#N)cn5)CC4)c3)c2c1 ; C#CCN(C)c1ccc2c(=O)[nH]nc(Cc3ccc(F)c(C(=O)N4CCN(c5ccc(C#N)cn5)CC4)c3)c2c1 ; N#Cc1ccc(N2CCN(C(=O)c3cc(Cc4n[nH]c(=O)c5ccc(N6CC(F)(F)C6)cc45)ccc3F)CC2)nc1 ; CC#CCOc1ccc2c(=O)[nH]nc(Cc3ccc(F)c(C(=O)N4CCN(c5ccc(C#N)cn5)CC4)c3)c2c1]

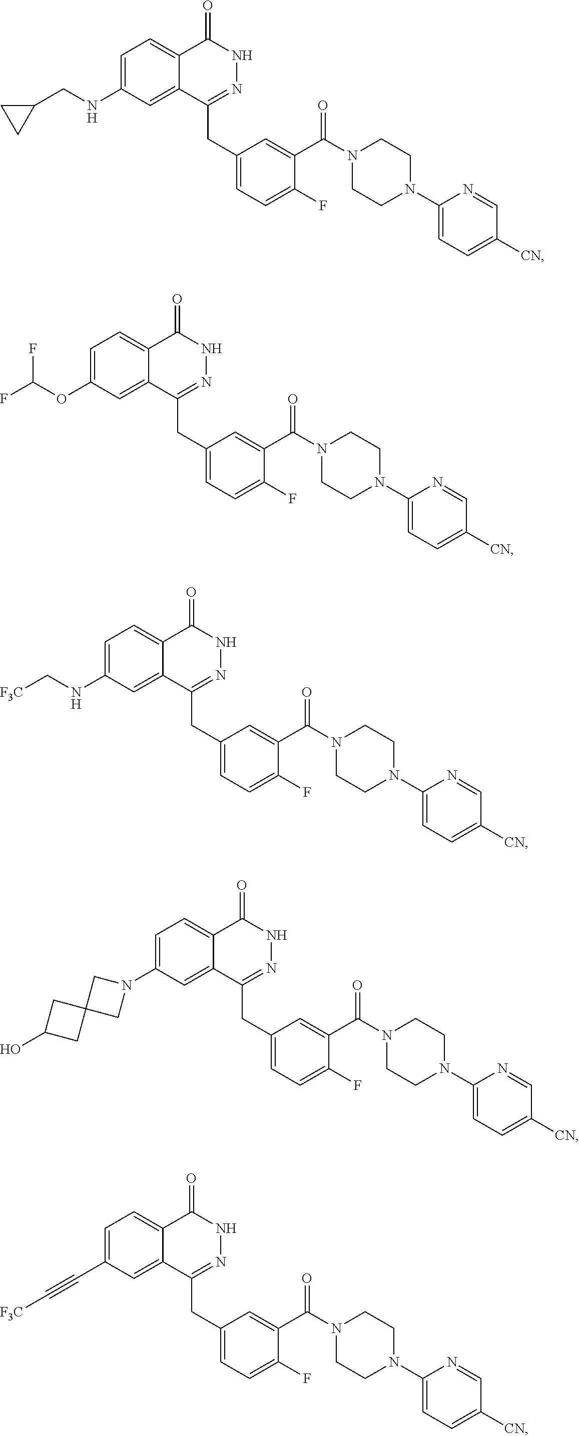 [SMILES: N#Cc1ccc(N2CCN(C(=O)c3cc(Cc4n[nH]c(=O)c5ccc(NCC6CC6)cc45)ccc3F)CC2)nc1 ; N#Cc1ccc(N2CCN(C(=O)c3cc(Cc4n[nH]c(=O)c5ccc(OC(F)F)cc45)ccc3F)CC2)nc1 ; N#Cc1ccc(N2CCN(C(=O)c3cc(Cc4n[nH]c(=O)c5ccc(NCC(F)(F)F)cc45)ccc3F)CC2)nc1 ; N#Cc1ccc(N2CCN(C(=O)c3cc(Cc4n[nH]c(=O)c5ccc(N6CC7(CC(O)C7)C6)cc45)ccc3F)CC2)nc1 ; N#Cc1ccc(N2CCN(C(=O)c3cc(Cc4n[nH]c(=O)c5ccc(C#CC(F)(F)F)cc45)ccc3F)CC2)nc1]

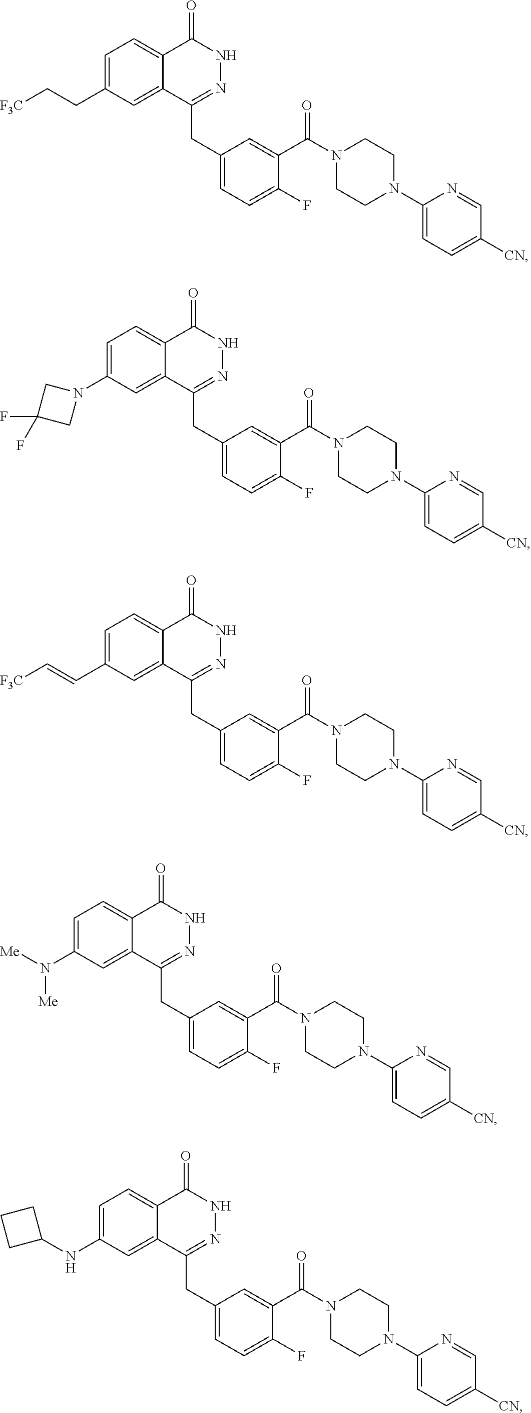 [SMILES: N#Cc1ccc(N2CCN(C(=O)c3cc(Cc4n[nH]c(=O)c5ccc(CCC(F)(F)F)cc45)ccc3F)CC2)nc1 ; N#Cc1ccc(N2CCN(C(=O)c3cc(Cc4n[nH]c(=O)c5ccc(N6CC(F)(F)C6)cc45)ccc3F)CC2)nc1 ; N#Cc1ccc(N2CCN(C(=O)c3cc(Cc4n[nH]c(=O)c5ccc(/C=C/C(F)(F)F)cc45)ccc3F)CC2)nc1 ; CN(C)c1ccc2c(=O)[nH]nc(Cc3ccc(F)c(C(=O)N4CCN(c5ccc(C#N)cn5)CC4)c3)c2c1 ; N#Cc1ccc(N2CCN(C(=O)c3cc(Cc4n[nH]c(=O)c5ccc(NC6CCC6)cc45)ccc3F)CC2)nc1]

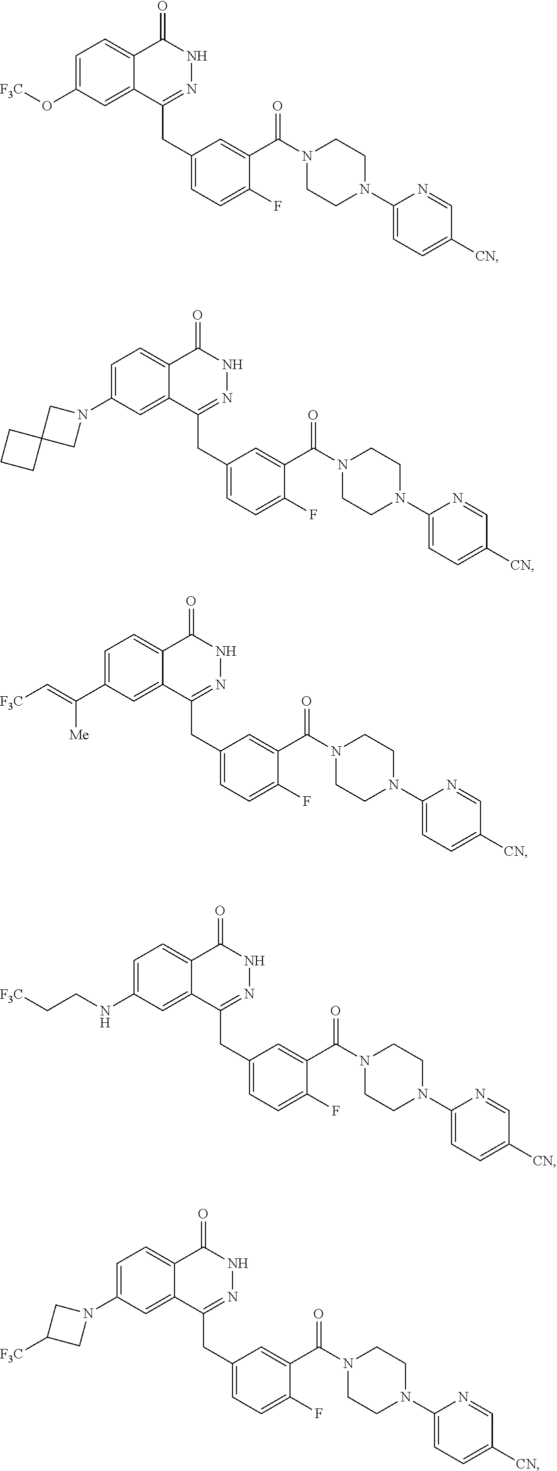 [SMILES: N#Cc1ccc(N2CCN(C(=O)c3cc(Cc4n[nH]c(=O)c5ccc(OC(F)(F)F)cc45)ccc3F)CC2)nc1 ; N#Cc1ccc(N2CCN(C(=O)c3cc(Cc4n[nH]c(=O)c5ccc(N6CC7(CCC7)C6)cc45)ccc3F)CC2)nc1 ; C/C(=C\C(F)(F)F)c1ccc2c(=O)[nH]nc(Cc3ccc(F)c(C(=O)N4CCN(c5ccc(C#N)cn5)CC4)c3)c2c1 ; N#Cc1ccc(N2CCN(C(=O)c3cc(Cc4n[nH]c(=O)c5ccc(NCCC(F)(F)F)cc45)ccc3F)CC2)nc1 ; N#Cc1ccc(N2CCN(C(=O)c3cc(Cc4n[nH]c(=O)c5ccc(N6CC(C(F)(F)F)C6)cc45)ccc3F)CC2)nc1]

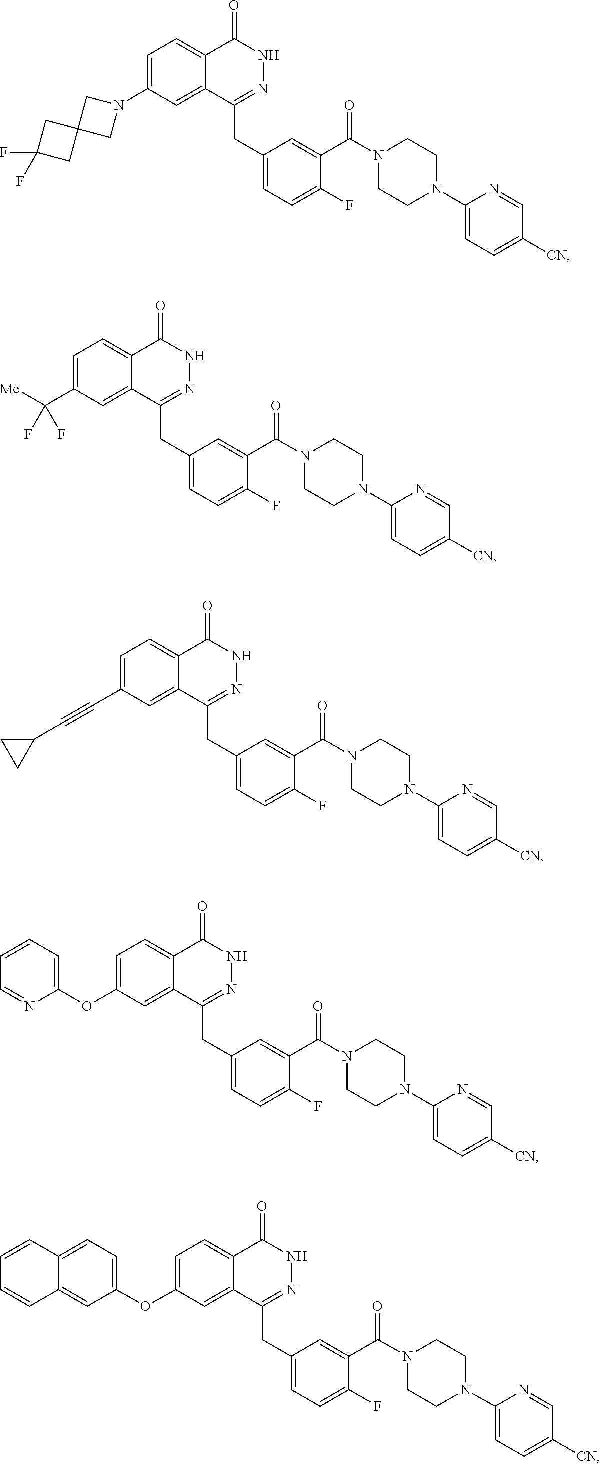 [SMILES: N#Cc1ccc(N2CCN(C(=O)c3cc(Cc4n[nH]c(=O)c5ccc(N6CC7(C6)CC(F)(F)C7)cc45)ccc3F)CC2)nc1 ; CC(F)(F)c1ccc2c(=O)[nH]nc(Cc3ccc(F)c(C(=O)N4CCN(c5ccc(C#N)cn5)CC4)c3)c2c1 ; N#Cc1ccc(N2CCN(C(=O)c3cc(Cc4n[nH]c(=O)c5ccc(C#CC6CC6)cc45)ccc3F)CC2)nc1 ; N#Cc1ccc(N2CCN(C(=O)c3cc(Cc4n[nH]c(=O)c5ccc(Oc6ccccn6)cc45)ccc3F)CC2)nc1 ; N#Cc1ccc(N2CCN(C(=O)c3cc(Cc4n[nH]c(=O)c5ccc(Oc6ccc7ccccc7c6)cc45)ccc3F)CC2)nc1]

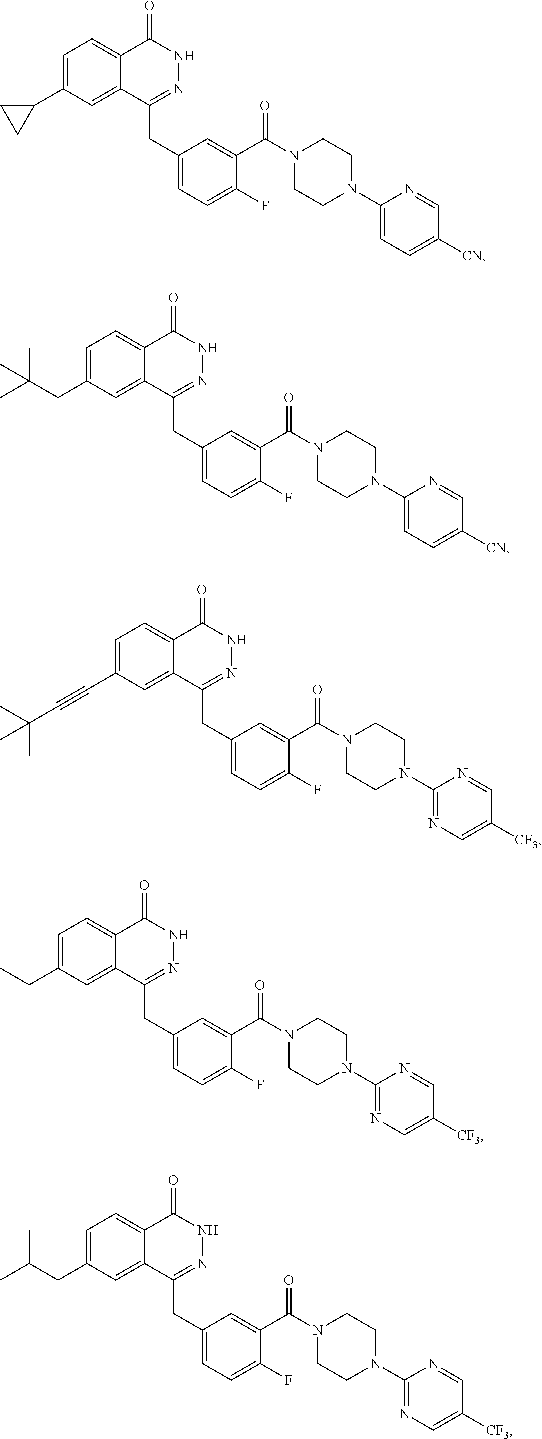 [SMILES: N#Cc1ccc(N2CCN(C(=O)c3cc(Cc4n[nH]c(=O)c5ccc(C6CC6)cc45)ccc3F)CC2)nc1 ; CC(C)(C)Cc1ccc2c(=O)[nH]nc(Cc3ccc(F)c(C(=O)N4CCN(c5ccc(C#N)cn5)CC4)c3)c2c1 ; CC(C)(C)C#Cc1ccc2c(=O)[nH]nc(Cc3ccc(F)c(C(=O)N4CCN(c5ncc(C(F)(F)F)cn5)CC4)c3)c2c1 ; CCc1ccc2c(=O)[nH]nc(Cc3ccc(F)c(C(=O)N4CCN(c5ncc(C(F)(F)F)cn5)CC4)c3)c2c1 ; CC(C)Cc1ccc2c(=O)[nH]nc(Cc3ccc(F)c(C(=O)N4CCN(c5ncc(C(F)(F)F)cn5)CC4)c3)c2c1]

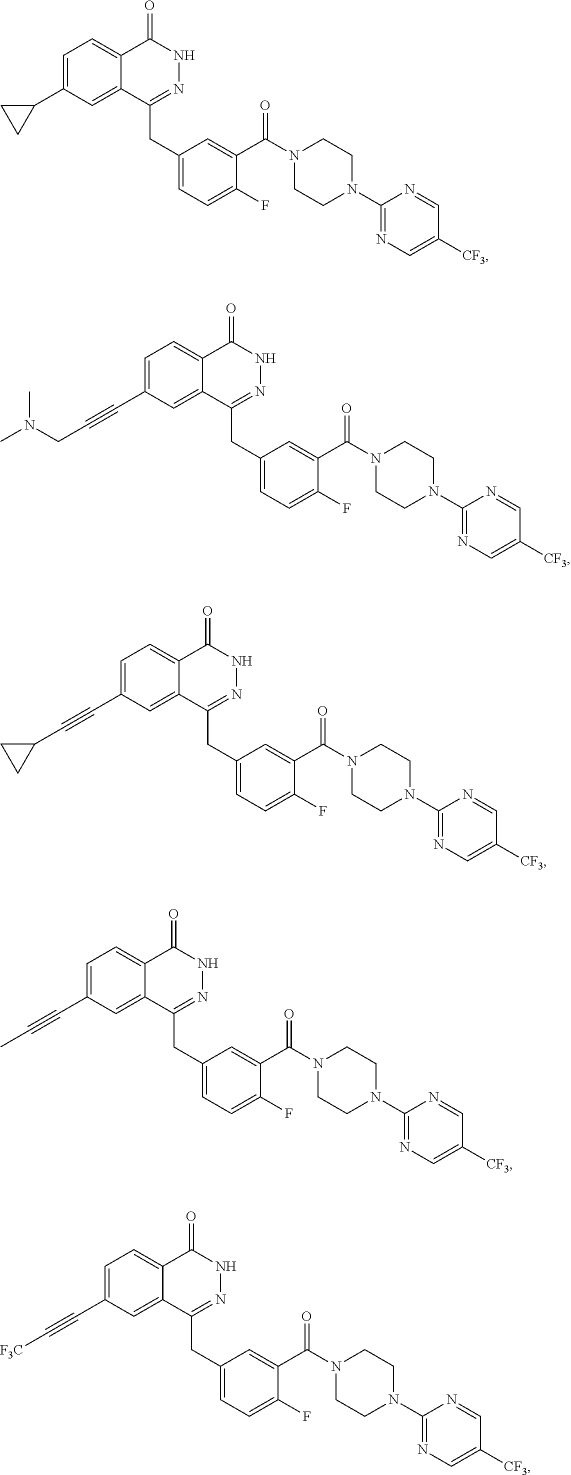 [SMILES: O=C(c1cc(Cc2n[nH]c(=O)c3ccc(C4CC4)cc23)ccc1F)N1CCN(c2ncc(C(F)(F)F)cn2)CC1 ; CN(C)CC#Cc1ccc2c(=O)[nH]nc(Cc3ccc(F)c(C(=O)N4CCN(c5ncc(C(F)(F)F)cn5)CC4)c3)c2c1 ; O=C(c1cc(Cc2n[nH]c(=O)c3ccc(C#CC4CC4)cc23)ccc1F)N1CCN(c2ncc(C(F)(F)F)cn2)CC1 ; CC#Cc1ccc2c(=O)[nH]nc(Cc3ccc(F)c(C(=O)N4CCN(c5ncc(C(F)(F)F)cn5)CC4)c3)c2c1 ; O=C(c1cc(Cc2n[nH]c(=O)c3ccc(C#CC(F)(F)F)cc23)ccc1F)N1CCN(c2ncc(C(F)(F)F)cn2)CC1]

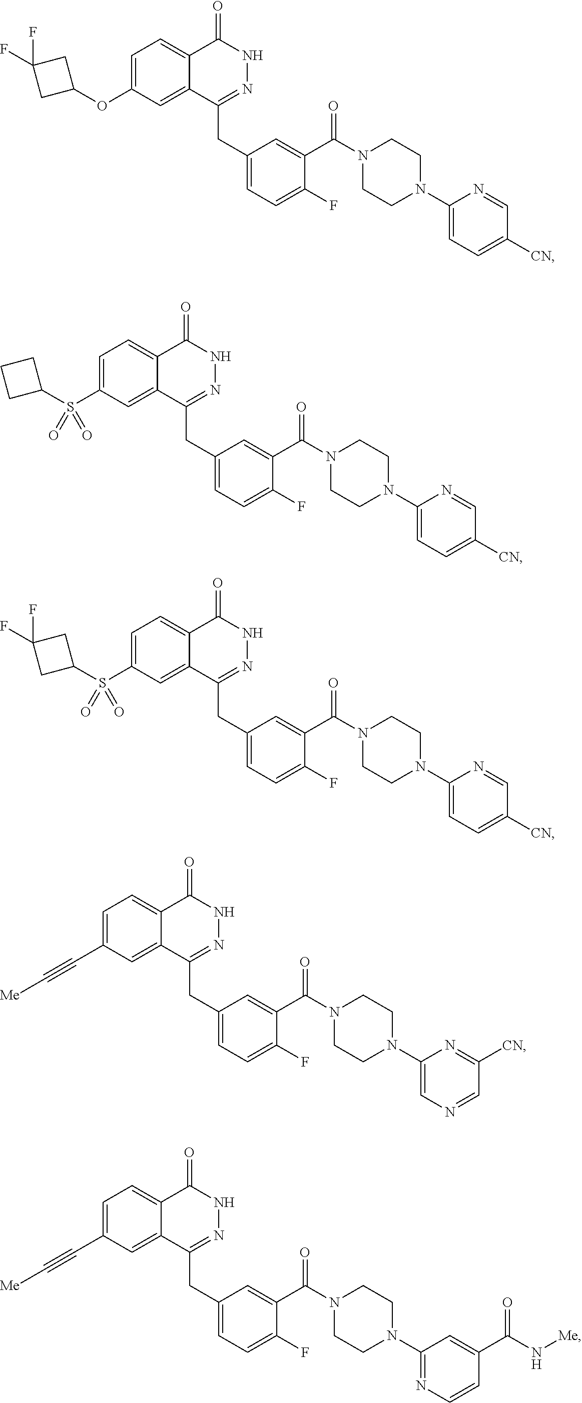 [SMILES: N#Cc1ccc(N2CCN(C(=O)c3cc(Cc4n[nH]c(=O)c5ccc(OC6CC(F)(F)C6)cc45)ccc3F)CC2)nc1 ; N#Cc1ccc(N2CCN(C(=O)c3cc(Cc4n[nH]c(=O)c5ccc(S(=O)(=O)C6CCC6)cc45)ccc3F)CC2)nc1 ; N#Cc1ccc(N2CCN(C(=O)c3cc(Cc4n[nH]c(=O)c5ccc(S(=O)(=O)C6CC(F)(F)C6)cc45)ccc3F)CC2)nc1 ; CC#Cc1ccc2c(=O)[nH]nc(Cc3ccc(F)c(C(=O)N4CCN(c5cncc(C#N)n5)CC4)c3)c2c1 ; CC#Cc1ccc2c(=O)[nH]nc(Cc3ccc(F)c(C(=O)N4CCN(c5cc(C(=O)NC)ccn5)CC4)c3)c2c1]

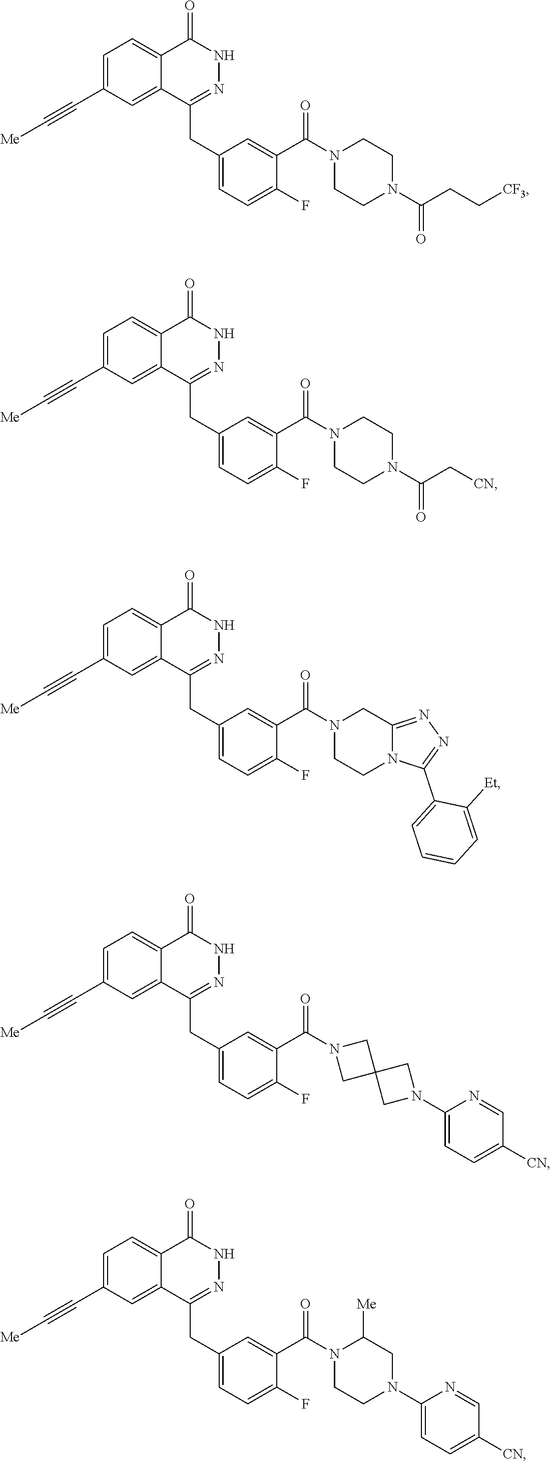 [SMILES: CC#Cc1ccc2c(=O)[nH]nc(Cc3ccc(F)c(C(=O)N4CCN(C(=O)CCC(F)(F)F)CC4)c3)c2c1 ; CC#Cc1ccc2c(=O)[nH]nc(Cc3ccc(F)c(C(=O)N4CCN(C(=O)CC#N)CC4)c3)c2c1 ; CC#Cc1ccc2c(=O)[nH]nc(Cc3ccc(F)c(C(=O)N4CCn5c(nnc5-c5ccccc5CC)C4)c3)c2c1 ; CC#Cc1ccc2c(=O)[nH]nc(Cc3ccc(F)c(C(=O)N4CC5(C4)CN(c4ccc(C#N)cn4)C5)c3)c2c1 ; CC#Cc1ccc2c(=O)[nH]nc(Cc3ccc(F)c(C(=O)N4CCN(c5ccc(C#N)cn5)CC4C)c3)c2c1]

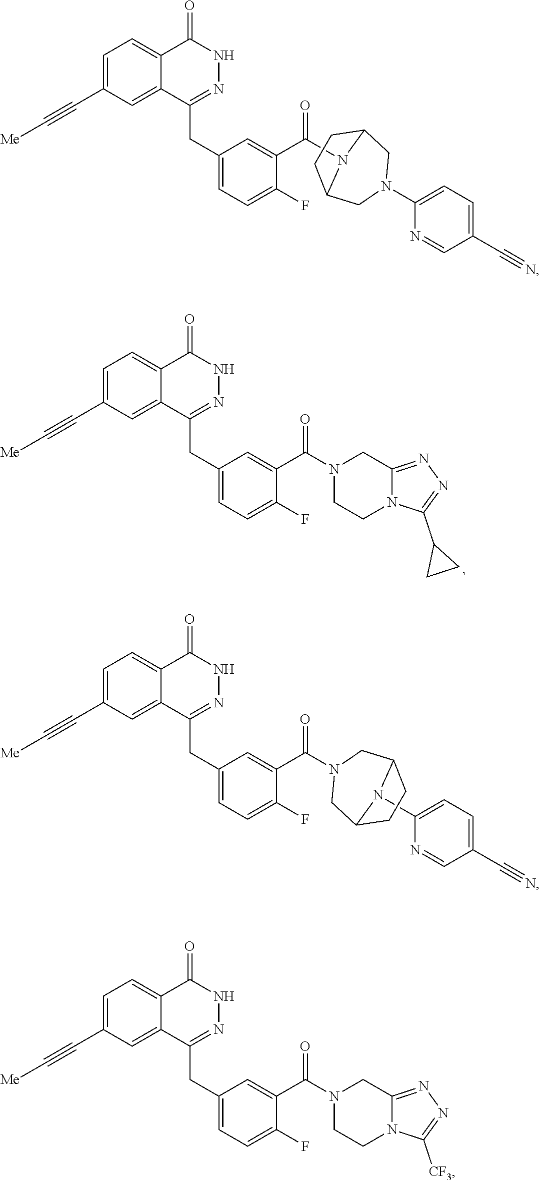 [SMILES: CC#Cc1ccc2c(=O)[nH]nc(Cc3ccc(F)c(C(=O)N4C5CCC4CN(c4ccc(C#N)cn4)C5)c3)c2c1 ; CC#Cc1ccc2c(=O)[nH]nc(Cc3ccc(F)c(C(=O)N4CCn5c(nnc5C5CC5)C4)c3)c2c1 ; CC#Cc1ccc2c(=O)[nH]nc(Cc3ccc(F)c(C(=O)N4CC5CCC(C4)N5c4ccc(C#N)cn4)c3)c2c1 ; CC#Cc1ccc2c(=O)[nH]nc(Cc3ccc(F)c(C(=O)N4CCn5c(nnc5C(F)(F)F)C4)c3)c2c1]

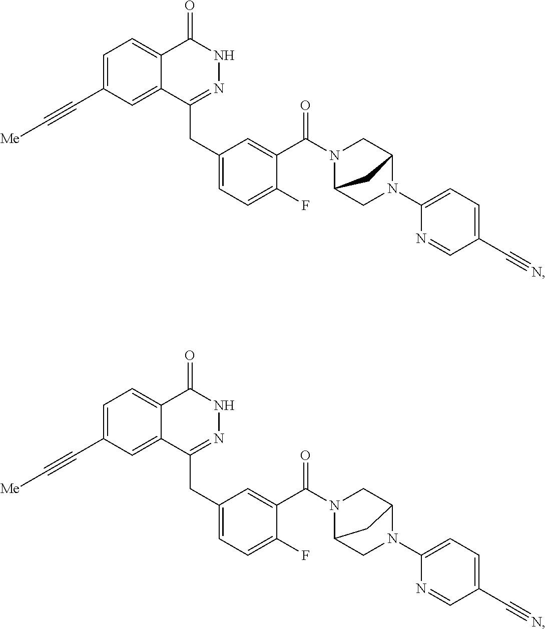 [SMILES: CC#Cc1ccc2c(=O)[nH]nc(Cc3ccc(F)c(C(=O)N4C[C@H]5C[C@@H]4CN5c4ccc(C#N)cn4)c3)c2c1 ; CC#Cc1ccc2c(=O)[nH]nc(Cc3ccc(F)c(C(=O)N4CC5CC4CN5c4ccc(C#N)cn4)c3)c2c1]

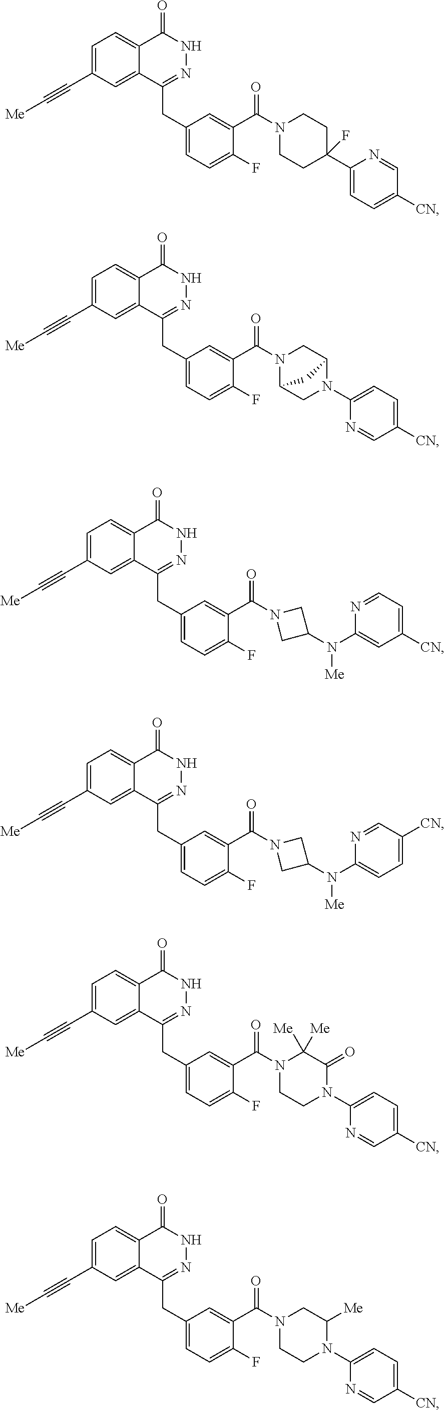 [SMILES: CC#Cc1ccc2c(=O)[nH]nc(Cc3ccc(F)c(C(=O)N4CCC(F)(c5ccc(C#N)cn5)CC4)c3)c2c1 ; CC#Cc1ccc2c(=O)[nH]nc(Cc3ccc(F)c(C(=O)N4C[C@@H]5C[C@H]4CN5c4ccc(C#N)cn4)c3)c2c1 ; CC#Cc1ccc2c(=O)[nH]nc(Cc3ccc(F)c(C(=O)N4CC(N(C)c5cc(C#N)ccn5)C4)c3)c2c1 ; CC#Cc1ccc2c(=O)[nH]nc(Cc3ccc(F)c(C(=O)N4CC(N(C)c5ccc(C#N)cn5)C4)c3)c2c1 ; CC#Cc1ccc2c(=O)[nH]nc(Cc3ccc(F)c(C(=O)N4CCN(c5ccc(C#N)cn5)C(=O)C4(C)C)c3)c2c1 ; CC#Cc1ccc2c(=O)[nH]nc(Cc3ccc(F)c(C(=O)N4CCN(c5ccc(C#N)cn5)C(C)C4)c3)c2c1]

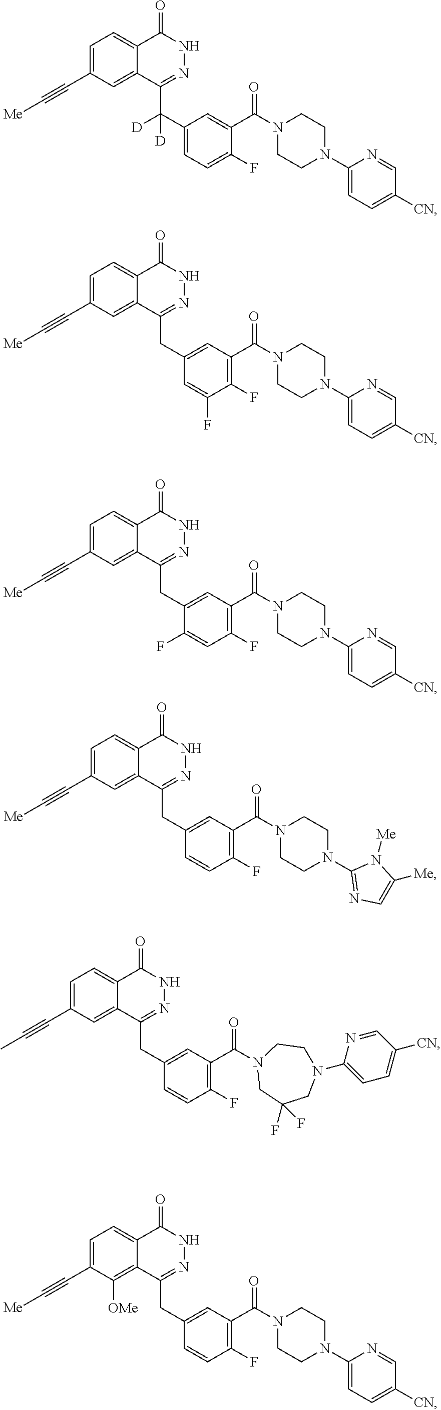 [SMILES: [2H]C([2H])(c1ccc(F)c(C(=O)N2CCN(c3ccc(C#N)cn3)CC2)c1)c1n[nH]c(=O)c2ccc(C#CC)cc12 ; CC#Cc1ccc2c(=O)[nH]nc(Cc3cc(F)c(F)c(C(=O)N4CCN(c5ccc(C#N)cn5)CC4)c3)c2c1 ; CC#Cc1ccc2c(=O)[nH]nc(Cc3cc(C(=O)N4CCN(c5ccc(C#N)cn5)CC4)c(F)cc3F)c2c1 ; CC#Cc1ccc2c(=O)[nH]nc(Cc3ccc(F)c(C(=O)N4CCN(c5ncc(C)n5C)CC4)c3)c2c1 ; CC#Cc1ccc2c(=O)[nH]nc(Cc3ccc(F)c(C(=O)N4CCN(c5ccc(C#N)cn5)CC(F)(F)C4)c3)c2c1 ; CC#Cc1ccc2c(=O)[nH]nc(Cc3ccc(F)c(C(=O)N4CCN(c5ccc(C#N)cn5)CC4)c3)c2c1OC]

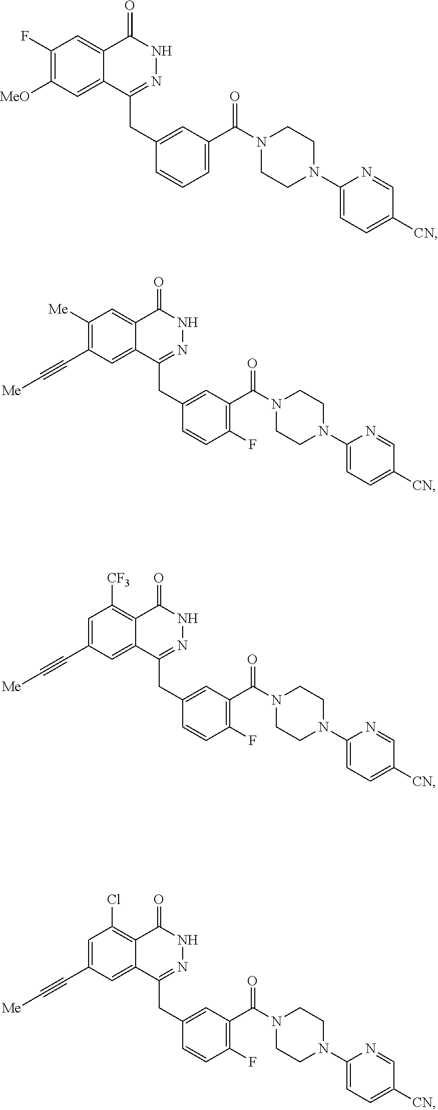 [SMILES: COc1cc2c(Cc3cccc(C(=O)N4CCN(c5ccc(C#N)cn5)CC4)c3)n[nH]c(=O)c2cc1F ; CC#Cc1cc2c(Cc3ccc(F)c(C(=O)N4CCN(c5ccc(C#N)cn5)CC4)c3)n[nH]c(=O)c2cc1C ; CC#Cc1cc(C(F)(F)F)c2c(=O)[nH]nc(Cc3ccc(F)c(C(=O)N4CCN(c5ccc(C#N)cn5)CC4)c3)c2c1 ; CC#Cc1cc(Cl)c2c(=O)[nH]nc(Cc3ccc(F)c(C(=O)N4CCN(c5ccc(C#N)cn5)CC4)c3)c2c1]

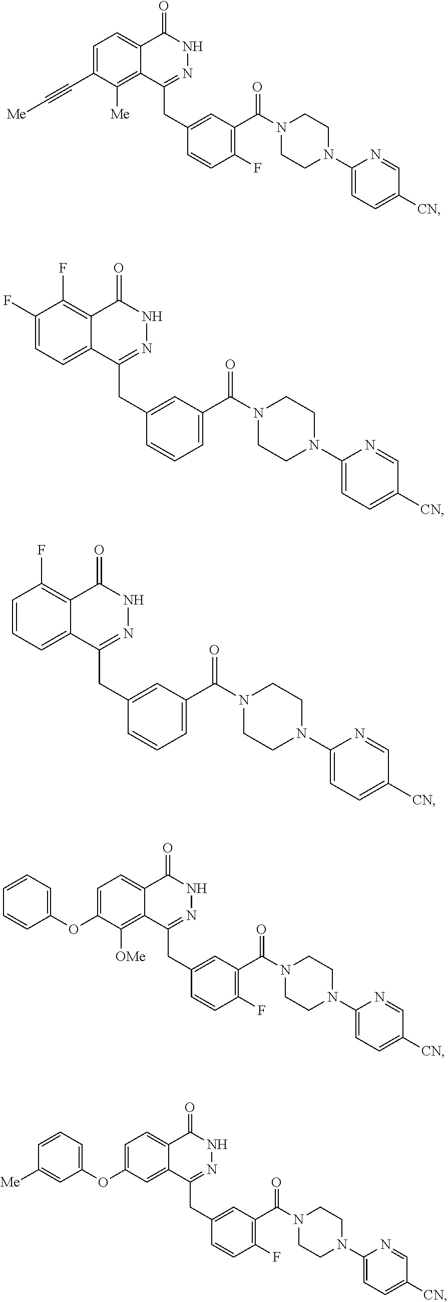 [SMILES: CC#Cc1ccc2c(=O)[nH]nc(Cc3ccc(F)c(C(=O)N4CCN(c5ccc(C#N)cn5)CC4)c3)c2c1C ; N#Cc1ccc(N2CCN(C(=O)c3cccc(Cc4n[nH]c(=O)c5c(F)c(F)ccc45)c3)CC2)nc1 ; N#Cc1ccc(N2CCN(C(=O)c3cccc(Cc4n[nH]c(=O)c5c(F)cccc45)c3)CC2)nc1 ; COc1c(Oc2ccccc2)ccc2c(=O)[nH]nc(Cc3ccc(F)c(C(=O)N4CCN(c5ccc(C#N)cn5)CC4)c3)c12 ; Cc1cccc(Oc2ccc3c(=O)[nH]nc(Cc4ccc(F)c(C(=O)N5CCN(c6ccc(C#N)cn6)CC5)c4)c3c2)c1]

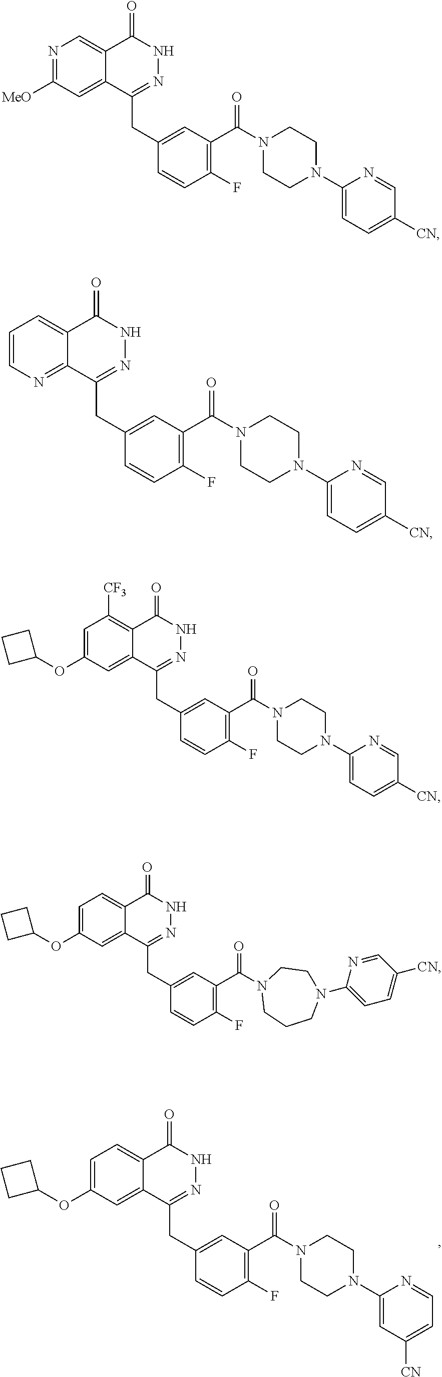 [SMILES: COc1cc2c(Cc3ccc(F)c(C(=O)N4CCN(c5ccc(C#N)cn5)CC4)c3)n[nH]c(=O)c2cn1 ; N#Cc1ccc(N2CCN(C(=O)c3cc(Cc4n[nH]c(=O)c5cccnc45)ccc3F)CC2)nc1 ; N#Cc1ccc(N2CCN(C(=O)c3cc(Cc4n[nH]c(=O)c5c(C(F)(F)F)cc(OC6CCC6)cc45)ccc3F)CC2)nc1 ; N#Cc1ccc(N2CCCN(C(=O)c3cc(Cc4n[nH]c(=O)c5ccc(OC6CCC6)cc45)ccc3F)CC2)nc1 ; N#Cc1ccnc(N2CCN(C(=O)c3cc(Cc4n[nH]c(=O)c5ccc(OC6CCC6)cc45)ccc3F)CC2)c1]

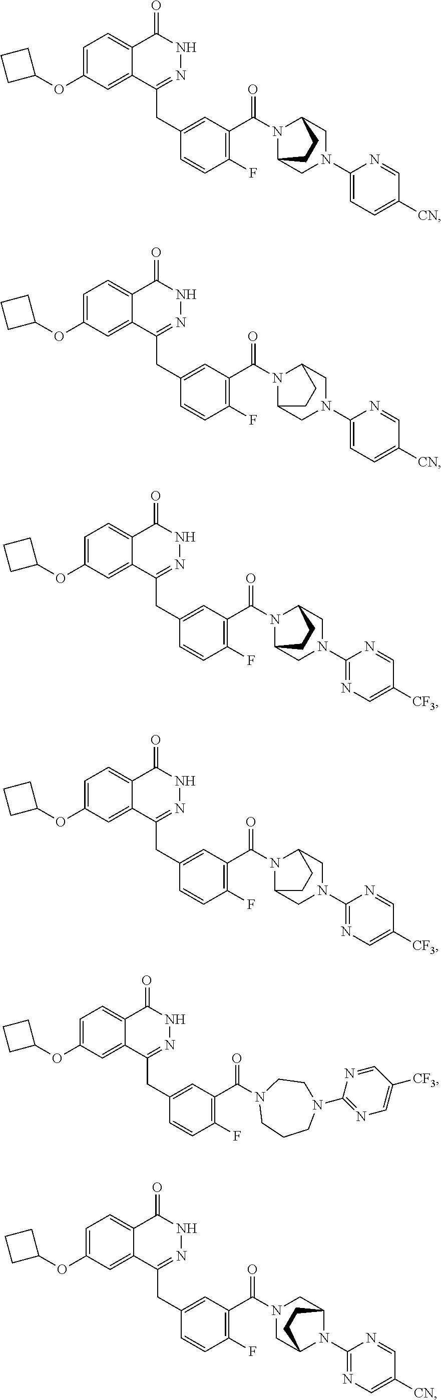

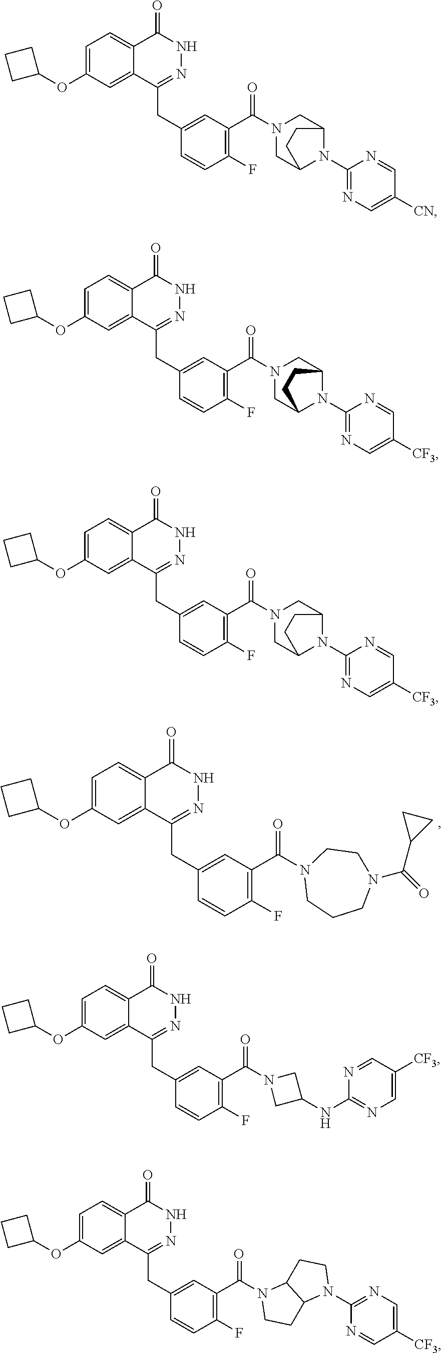

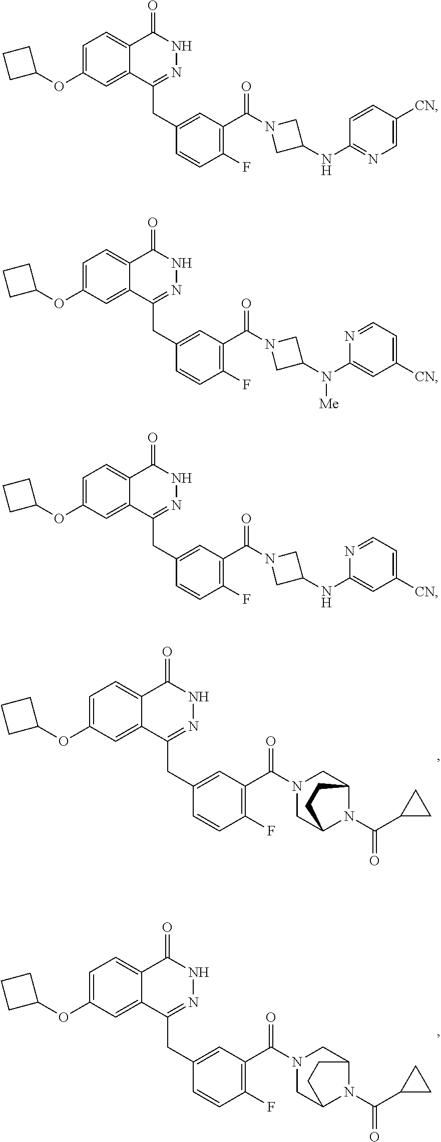

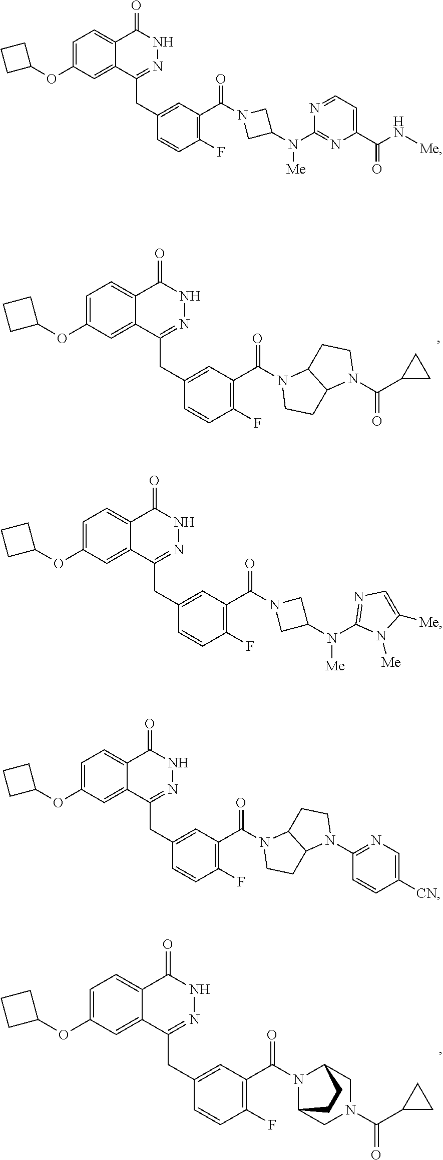

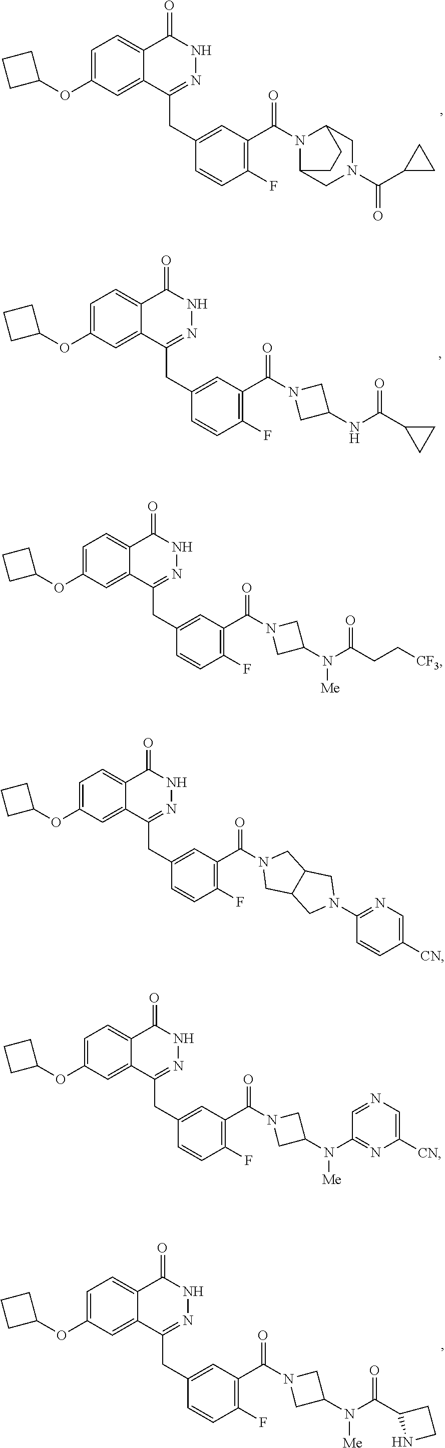

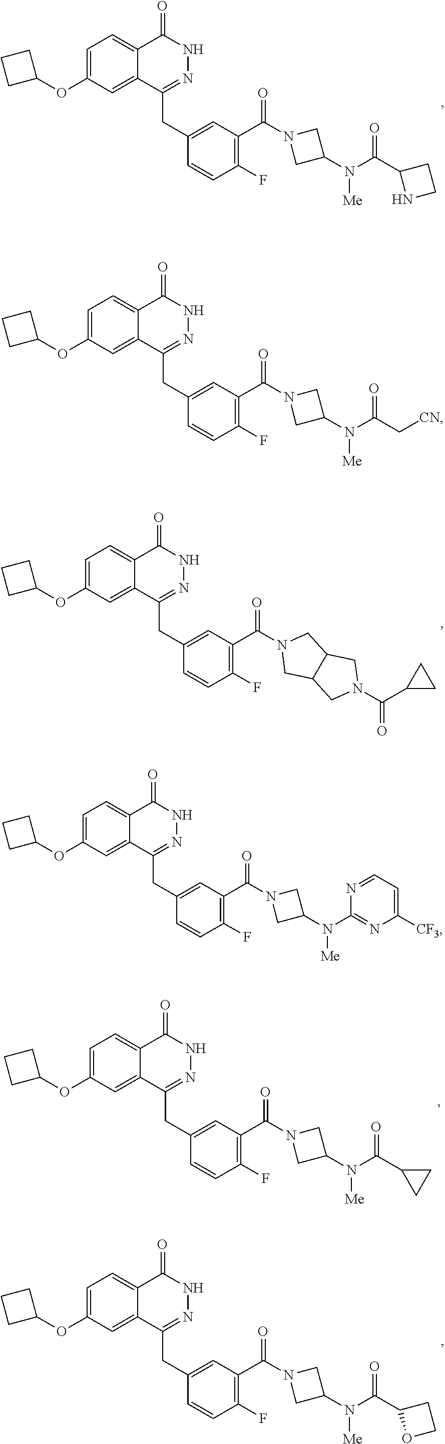

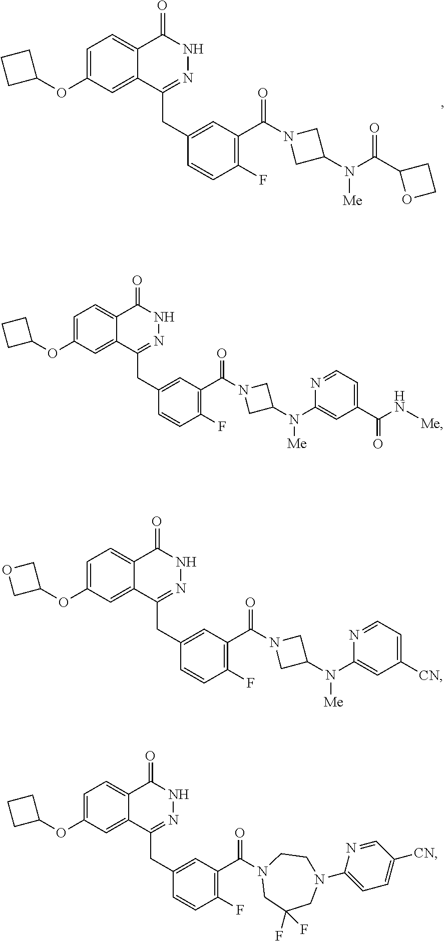 [SMILES: CN(C(=O)C1CCO1)C1CN(C(=O)c2cc(Cc3n[nH]c(=O)c4ccc(OC5CCC5)cc34)ccc2F)C1 ; CNC(=O)c1ccnc(N(C)C2CN(C(=O)c3cc(Cc4n[nH]c(=O)c5ccc(OC6CCC6)cc45)ccc3F)C2)c1 ; CN(c1cc(C#N)ccn1)C1CN(C(=O)c2cc(Cc3n[nH]c(=O)c4ccc(OC5COC5)cc34)ccc2F)C1 ; N#Cc1ccc(N2CCN(C(=O)c3cc(Cc4n[nH]c(=O)c5ccc(OC6CCC6)cc45)ccc3F)CC(F)(F)C2)nc1]

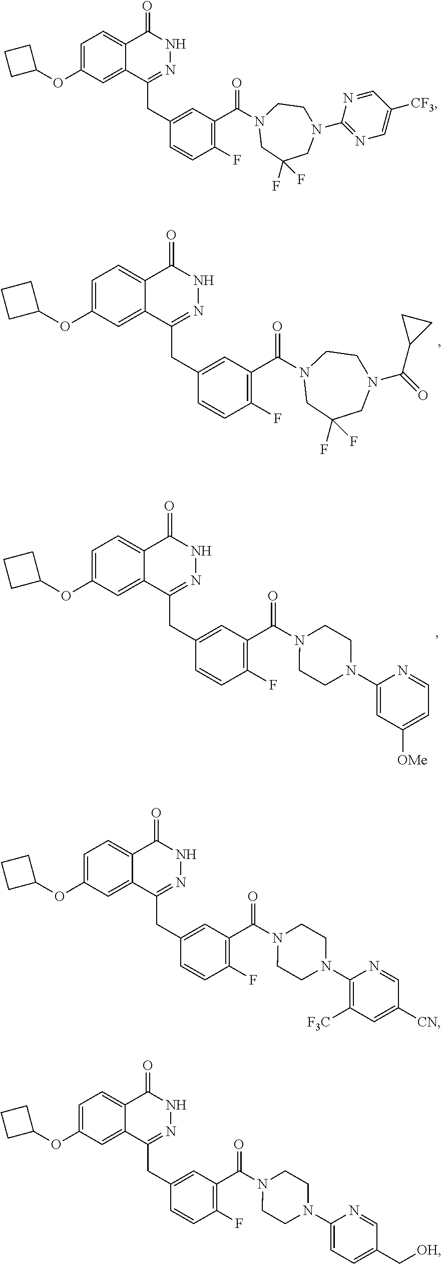 [SMILES: O=C(c1cc(Cc2n[nH]c(=O)c3ccc(OC4CCC4)cc23)ccc1F)N1CCN(c2ncc(C(F)(F)F)cn2)CC(F)(F)C1 ; O=C(c1cc(Cc2n[nH]c(=O)c3ccc(OC4CCC4)cc23)ccc1F)N1CCN(C(=O)C2CC2)CC(F)(F)C1 ; COc1ccnc(N2CCN(C(=O)c3cc(Cc4n[nH]c(=O)c5ccc(OC6CCC6)cc45)ccc3F)CC2)c1 ; N#Cc1cnc(N2CCN(C(=O)c3cc(Cc4n[nH]c(=O)c5ccc(OC6CCC6)cc45)ccc3F)CC2)c(C(F)(F)F)c1 ; O=C(c1cc(Cc2n[nH]c(=O)c3ccc(OC4CCC4)cc23)ccc1F)N1CCN(c2ccc(CO)cn2)CC1]

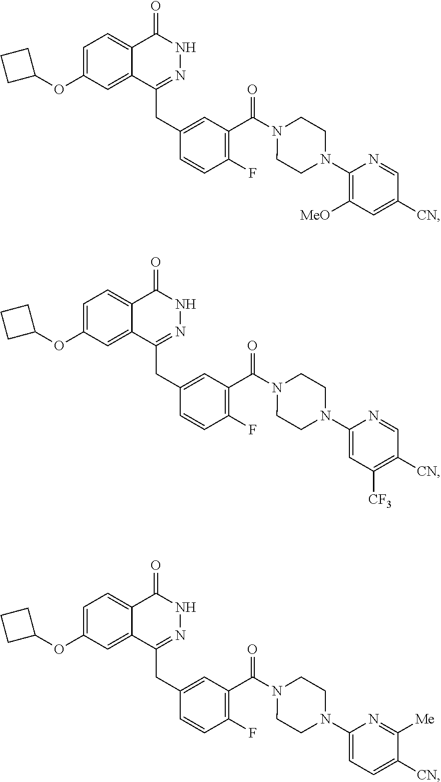 [SMILES: COc1cc(C#N)cnc1N1CCN(C(=O)c2cc(Cc3n[nH]c(=O)c4ccc(OC5CCC5)cc34)ccc2F)CC1 ; N#Cc1cnc(N2CCN(C(=O)c3cc(Cc4n[nH]c(=O)c5ccc(OC6CCC6)cc45)ccc3F)CC2)cc1C(F)(F)F ; Cc1nc(N2CCN(C(=O)c3cc(Cc4n[nH]c(=O)c5ccc(OC6CCC6)cc45)ccc3F)CC2)ccc1C#N]

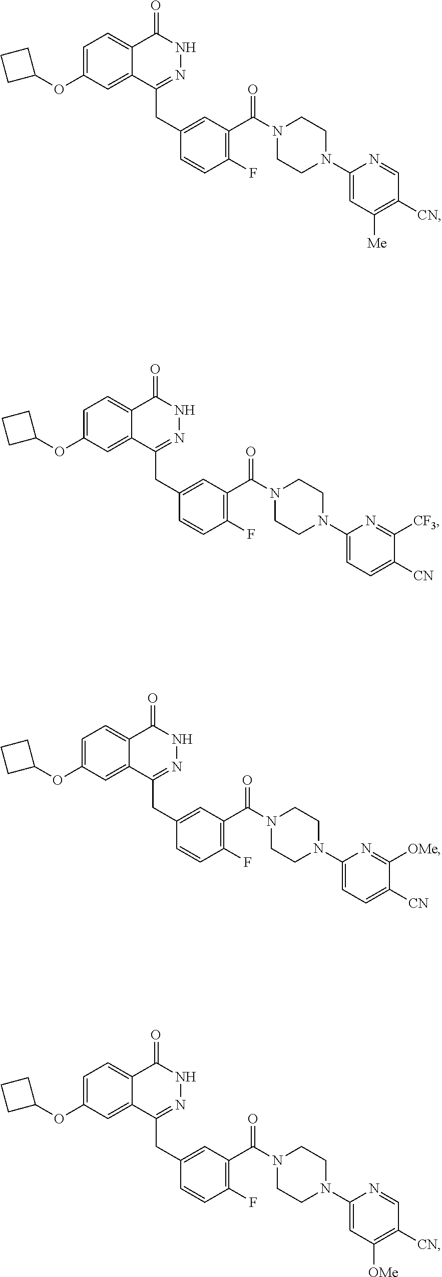 [SMILES: Cc1cc(N2CCN(C(=O)c3cc(Cc4n[nH]c(=O)c5ccc(OC6CCC6)cc45)ccc3F)CC2)ncc1C#N ; N#Cc1ccc(N2CCN(C(=O)c3cc(Cc4n[nH]c(=O)c5ccc(OC6CCC6)cc45)ccc3F)CC2)nc1C(F)(F)F ; COc1nc(N2CCN(C(=O)c3cc(Cc4n[nH]c(=O)c5ccc(OC6CCC6)cc45)ccc3F)CC2)ccc1C#N ; COc1cc(N2CCN(C(=O)c3cc(Cc4n[nH]c(=O)c5ccc(OC6CCC6)cc45)ccc3F)CC2)ncc1C#N]

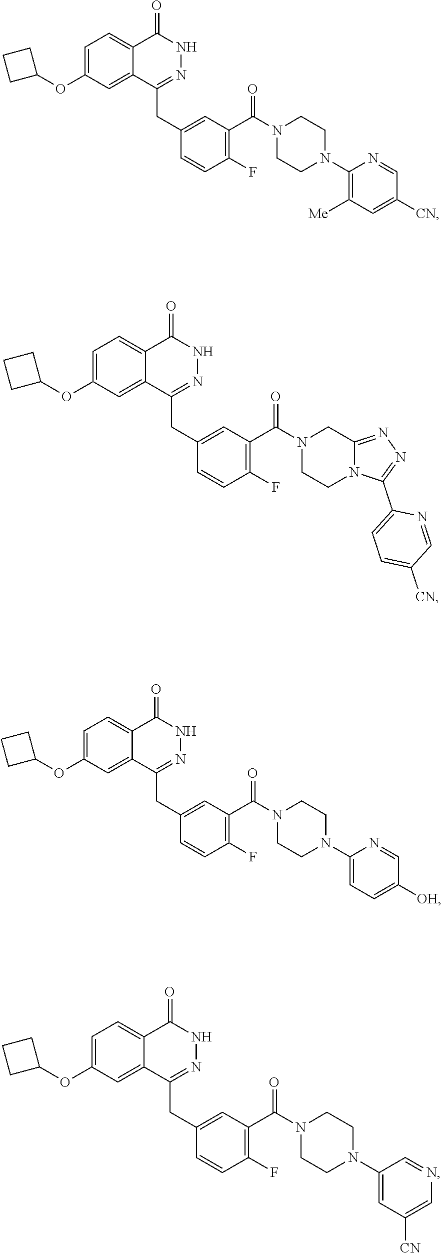 [SMILES: Cc1cc(C#N)cnc1N1CCN(C(=O)c2cc(Cc3n[nH]c(=O)c4ccc(OC5CCC5)cc34)ccc2F)CC1 ; N#Cc1ccc(-c2nnc3n2CCN(C(=O)c2cc(Cc4n[nH]c(=O)c5ccc(OC6CCC6)cc45)ccc2F)C3)nc1 ; O=C(c1cc(Cc2n[nH]c(=O)c3ccc(OC4CCC4)cc23)ccc1F)N1CCN(c2ccc(O)cn2)CC1 ; N#Cc1cncc(N2CCN(C(=O)c3cc(Cc4n[nH]c(=O)c5ccc(OC6CCC6)cc45)ccc3F)CC2)c1]

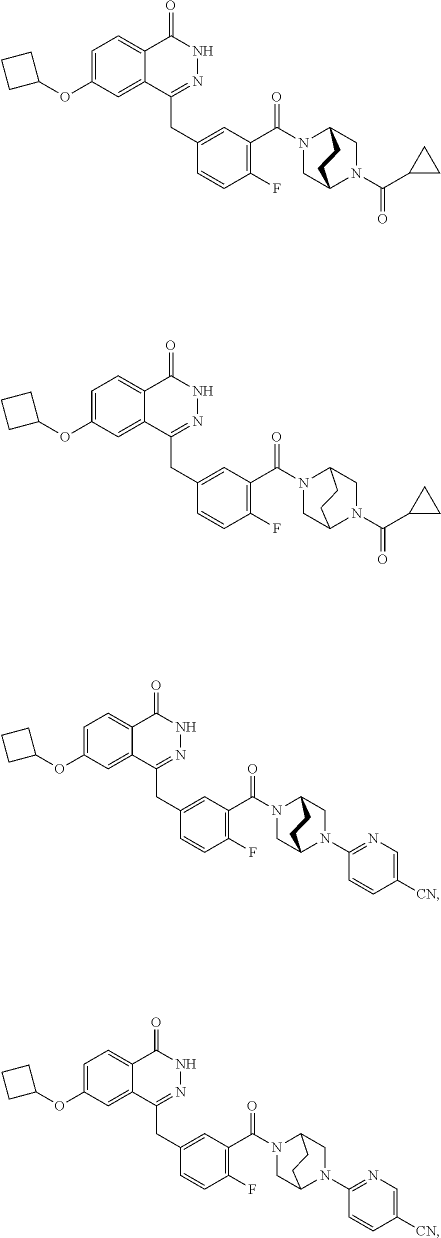

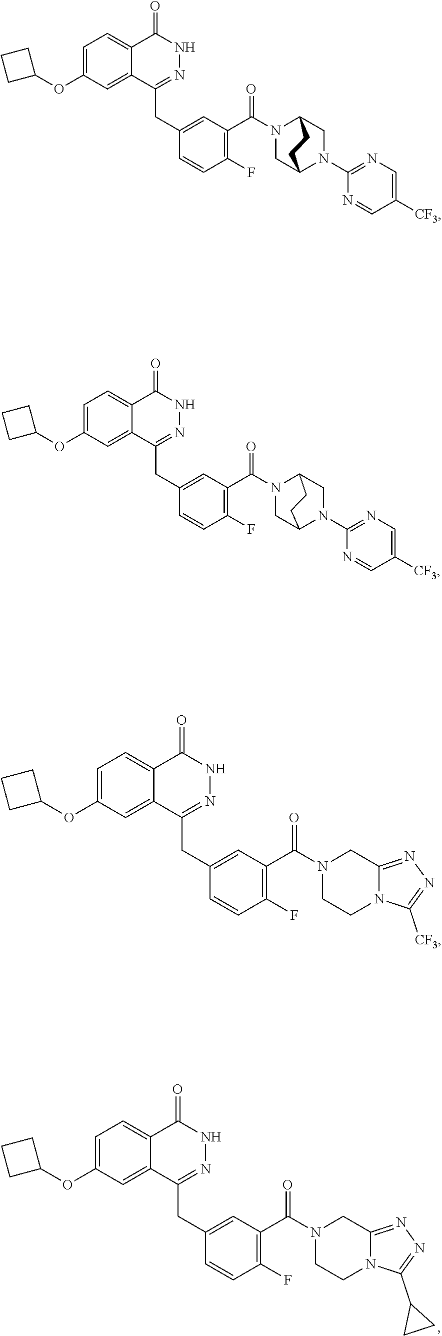

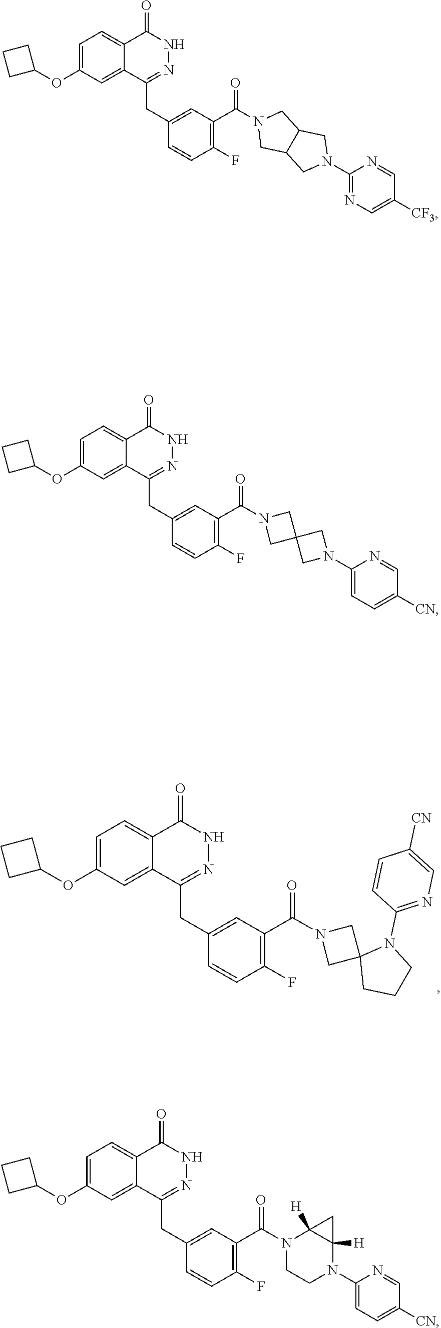 [SMILES: O=C(c1cc(Cc2n[nH]c(=O)c3ccc(OC4CCC4)cc23)ccc1F)N1CC2CN(c3ncc(C(F)(F)F)cn3)CC2C1 ; N#Cc1ccc(N2CC3(CN(C(=O)c4cc(Cc5n[nH]c(=O)c6ccc(OC7CCC7)cc56)ccc4F)C3)C2)nc1 ; N#Cc1ccc(N2CCCC23CN(C(=O)c2cc(Cc4n[nH]c(=O)c5ccc(OC6CCC6)cc45)ccc2F)C3)nc1 ; N#Cc1ccc(N2CCN(C(=O)c3cc(Cc4n[nH]c(=O)c5ccc(OC6CCC6)cc45)ccc3F)[C@@H]3C[C@@H]32)nc1]

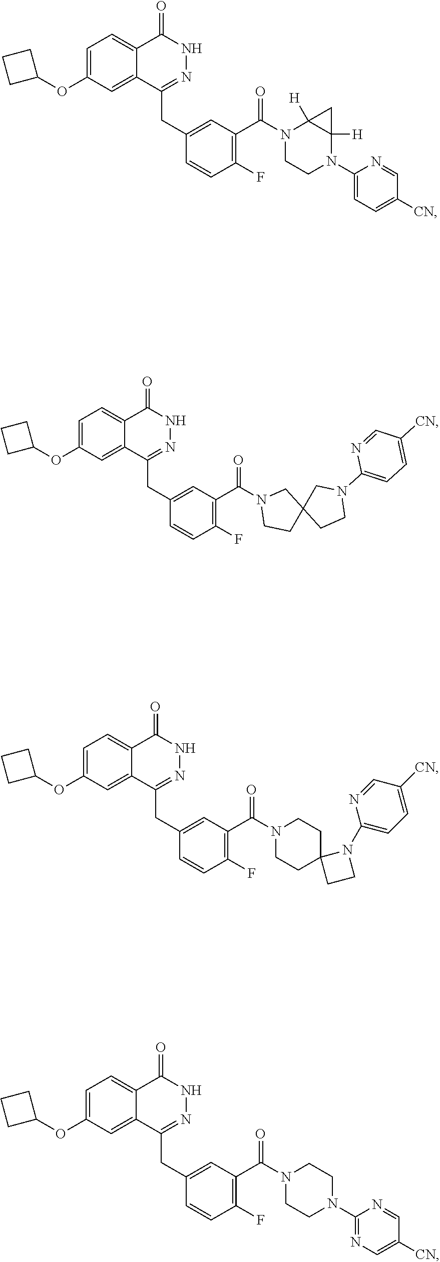 [SMILES: N#Cc1ccc(N2CCN(C(=O)c3cc(Cc4n[nH]c(=O)c5ccc(OC6CCC6)cc45)ccc3F)C3CC32)nc1 ; N#Cc1ccc(N2CCC3(CCN(C(=O)c4cc(Cc5n[nH]c(=O)c6ccc(OC7CCC7)cc56)ccc4F)C3)C2)nc1 ; N#Cc1ccc(N2CCC23CCN(C(=O)c2cc(Cc4n[nH]c(=O)c5ccc(OC6CCC6)cc45)ccc2F)CC3)nc1 ; N#Cc1cnc(N2CCN(C(=O)c3cc(Cc4n[nH]c(=O)c5ccc(OC6CCC6)cc45)ccc3F)CC2)nc1]

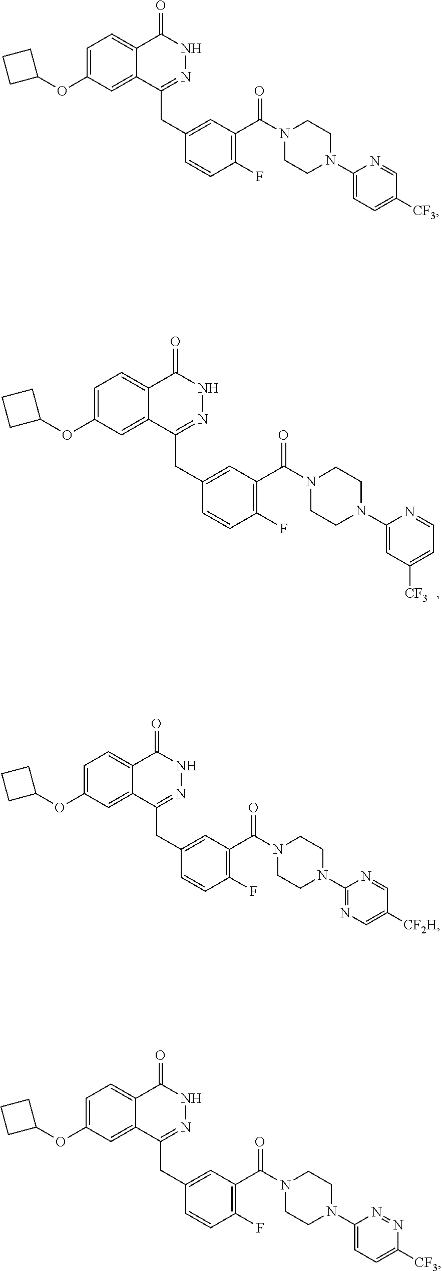 [SMILES: O=C(c1cc(Cc2n[nH]c(=O)c3ccc(OC4CCC4)cc23)ccc1F)N1CCN(c2ccc(C(F)(F)F)cn2)CC1 ; O=C(c1cc(Cc2n[nH]c(=O)c3ccc(OC4CCC4)cc23)ccc1F)N1CCN(c2cc(C(F)(F)F)ccn2)CC1 ; O=C(c1cc(Cc2n[nH]c(=O)c3ccc(OC4CCC4)cc23)ccc1F)N1CCN(c2ncc(C(F)F)cn2)CC1 ; O=C(c1cc(Cc2n[nH]c(=O)c3ccc(OC4CCC4)cc23)ccc1F)N1CCN(c2ccc(C(F)(F)F)nn2)CC1]

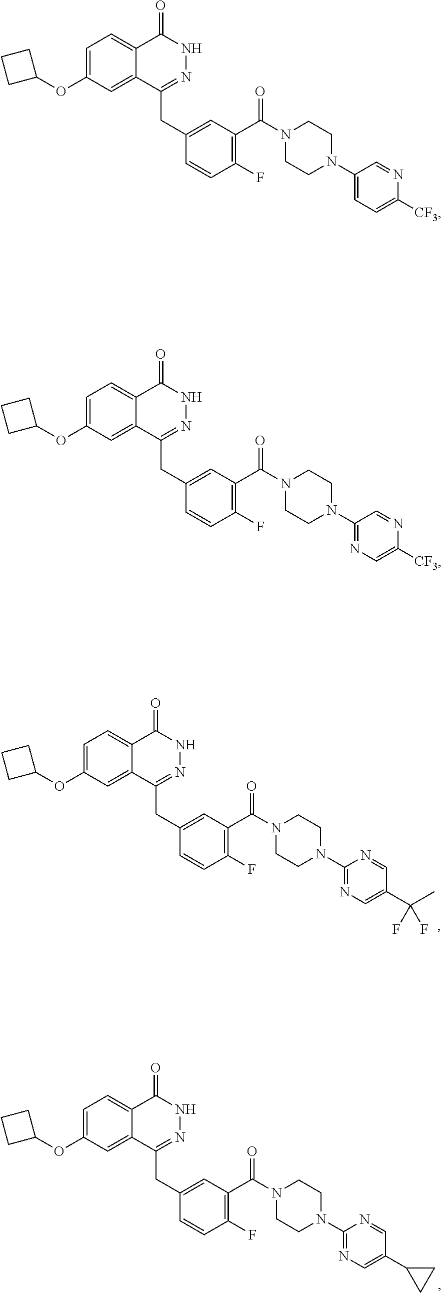 [SMILES: O=C(c1cc(Cc2n[nH]c(=O)c3ccc(OC4CCC4)cc23)ccc1F)N1CCN(c2ccc(C(F)(F)F)nc2)CC1 ; O=C(c1cc(Cc2n[nH]c(=O)c3ccc(OC4CCC4)cc23)ccc1F)N1CCN(c2cnc(C(F)(F)F)cn2)CC1 ; CC(F)(F)c1cnc(N2CCN(C(=O)c3cc(Cc4n[nH]c(=O)c5ccc(OC6CCC6)cc45)ccc3F)CC2)nc1 ; O=C(c1cc(Cc2n[nH]c(=O)c3ccc(OC4CCC4)cc23)ccc1F)N1CCN(c2ncc(C3CC3)cn2)CC1]

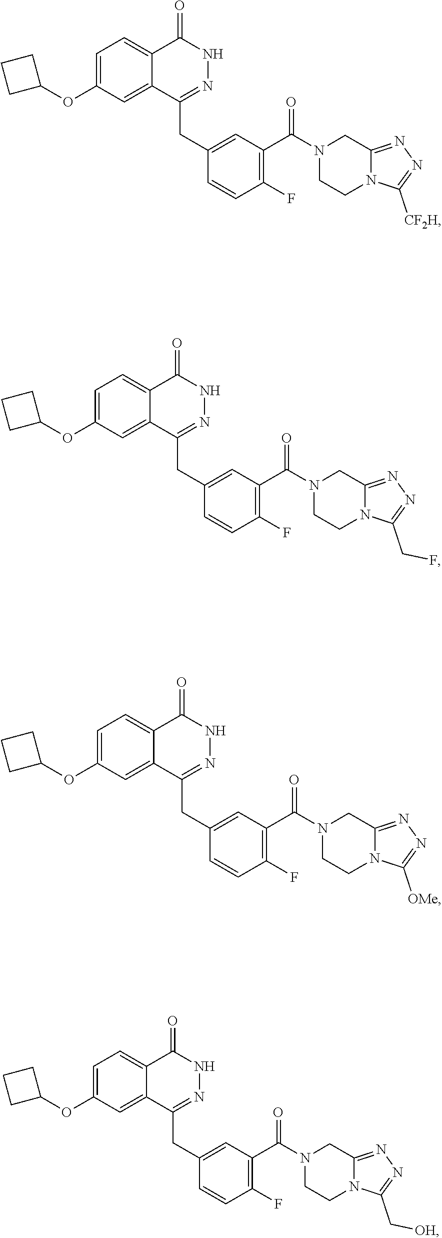 [SMILES: O=C(c1cc(Cc2n[nH]c(=O)c3ccc(OC4CCC4)cc23)ccc1F)N1CCn2c(nnc2C(F)F)C1 ; O=C(c1cc(Cc2n[nH]c(=O)c3ccc(OC4CCC4)cc23)ccc1F)N1CCn2c(CF)nnc2C1 ; COc1nnc2n1CCN(C(=O)c1cc(Cc3n[nH]c(=O)c4ccc(OC5CCC5)cc34)ccc1F)C2 ; O=C(c1cc(Cc2n[nH]c(=O)c3ccc(OC4CCC4)cc23)ccc1F)N1CCn2c(CO)nnc2C1]

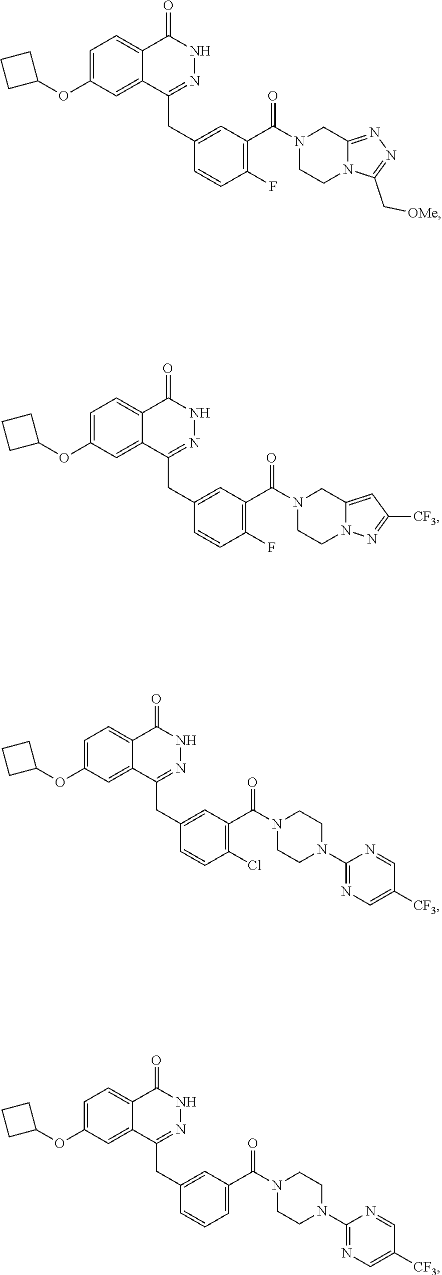 [SMILES: COCc1nnc2n1CCN(C(=O)c1cc(Cc3n[nH]c(=O)c4ccc(OC5CCC5)cc34)ccc1F)C2 ; O=C(c1cc(Cc2n[nH]c(=O)c3ccc(OC4CCC4)cc23)ccc1F)N1CCn2nc(C(F)(F)F)cc2C1 ; O=C(c1cc(Cc2n[nH]c(=O)c3ccc(OC4CCC4)cc23)ccc1Cl)N1CCN(c2ncc(C(F)(F)F)cn2)CC1 ; O=C(c1cccc(Cc2n[nH]c(=O)c3ccc(OC4CCC4)cc23)c1)N1CCN(c2ncc(C(F)(F)F)cn2)CC1]

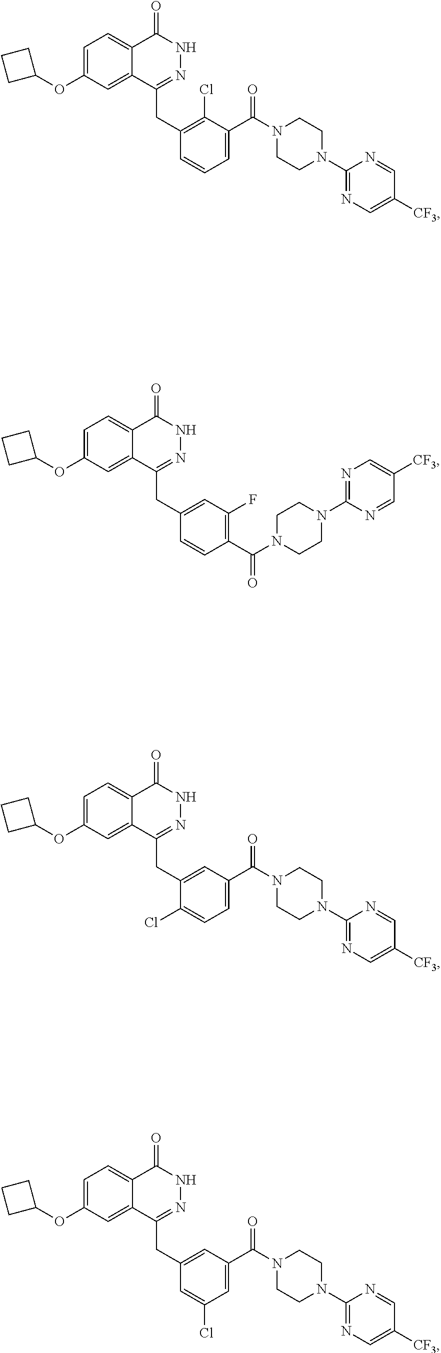 [SMILES: O=C(c1cccc(Cc2n[nH]c(=O)c3ccc(OC4CCC4)cc23)c1Cl)N1CCN(c2ncc(C(F)(F)F)cn2)CC1 ; O=C(c1ccc(Cc2n[nH]c(=O)c3ccc(OC4CCC4)cc23)cc1F)N1CCN(c2ncc(C(F)(F)F)cn2)CC1 ; O=C(c1ccc(Cl)c(Cc2n[nH]c(=O)c3ccc(OC4CCC4)cc23)c1)N1CCN(c2ncc(C(F)(F)F)cn2)CC1 ; O=C(c1cc(Cl)cc(Cc2n[nH]c(=O)c3ccc(OC4CCC4)cc23)c1)N1CCN(c2ncc(C(F)(F)F)cn2)CC1]

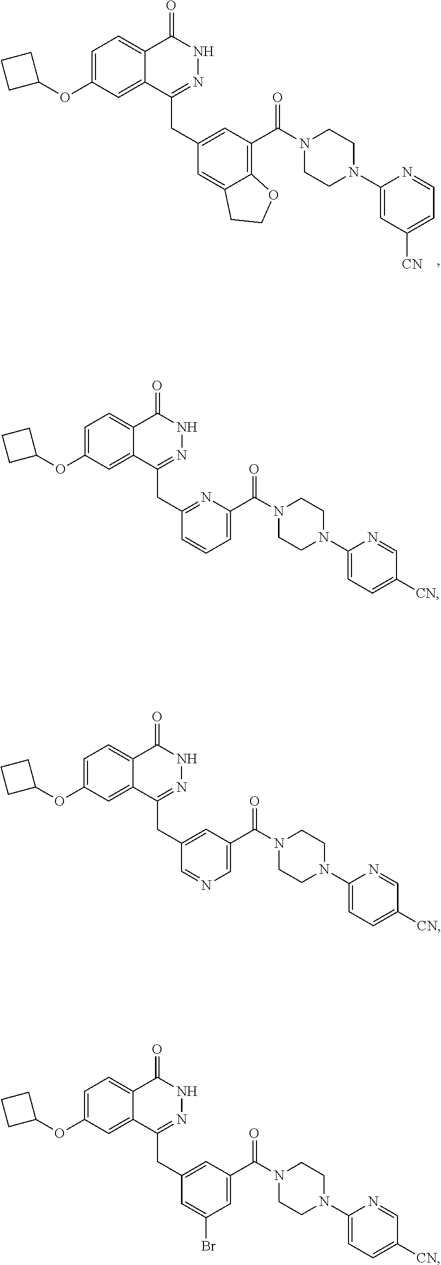 [SMILES: N#Cc1ccnc(N2CCN(C(=O)c3cc(Cc4n[nH]c(=O)c5ccc(OC6CCC6)cc45)cc4c3OCC4)CC2)c1 ; N#Cc1ccc(N2CCN(C(=O)c3cccc(Cc4n[nH]c(=O)c5ccc(OC6CCC6)cc45)n3)CC2)nc1 ; N#Cc1ccc(N2CCN(C(=O)c3cncc(Cc4n[nH]c(=O)c5ccc(OC6CCC6)cc45)c3)CC2)nc1 ; N#Cc1ccc(N2CCN(C(=O)c3cc(Br)cc(Cc4n[nH]c(=O)c5ccc(OC6CCC6)cc45)c3)CC2)nc1]

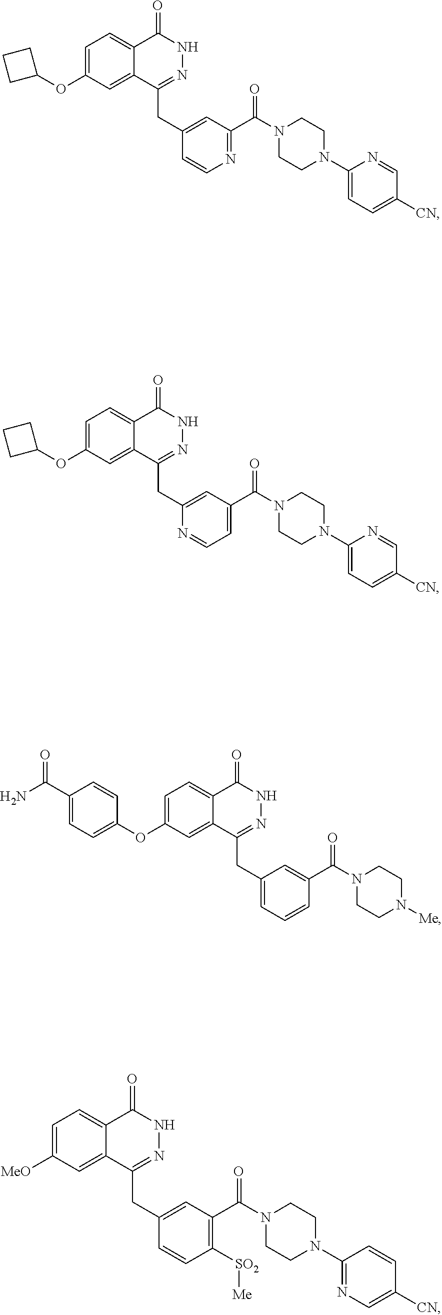 [SMILES: N#Cc1ccc(N2CCN(C(=O)c3cc(Cc4n[nH]c(=O)c5ccc(OC6CCC6)cc45)ccn3)CC2)nc1 ; N#Cc1ccc(N2CCN(C(=O)c3ccnc(Cc4n[nH]c(=O)c5ccc(OC6CCC6)cc45)c3)CC2)nc1 ; CN1CCN(C(=O)c2cccc(Cc3n[nH]c(=O)c4ccc(Oc5ccc(C(N)=O)cc5)cc34)c2)CC1 ; COc1ccc2c(=O)[nH]nc(Cc3ccc(S(C)(=O)=O)c(C(=O)N4CCN(c5ccc(C#N)cn5)CC4)c3)c2c1]

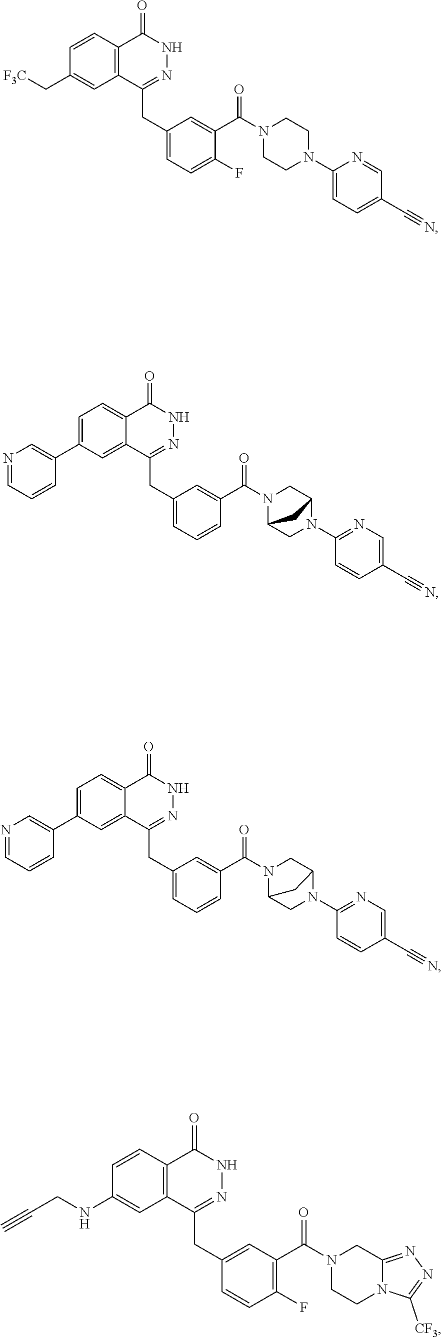 [SMILES: N#Cc1ccc(N2CCN(C(=O)c3cc(Cc4n[nH]c(=O)c5ccc(CC(F)(F)F)cc45)ccc3F)CC2)nc1 ; N#Cc1ccc(N2C[C@H]3C[C@@H]2CN3C(=O)c2cccc(Cc3n[nH]c(=O)c4ccc(-c5cccnc5)cc34)c2)nc1 ; N#Cc1ccc(N2CC3CC2CN3C(=O)c2cccc(Cc3n[nH]c(=O)c4ccc(-c5cccnc5)cc34)c2)nc1 ; C#CCNc1ccc2c(=O)[nH]nc(Cc3ccc(F)c(C(=O)N4CCn5c(nnc5C(F)(F)F)C4)c3)c2c1]

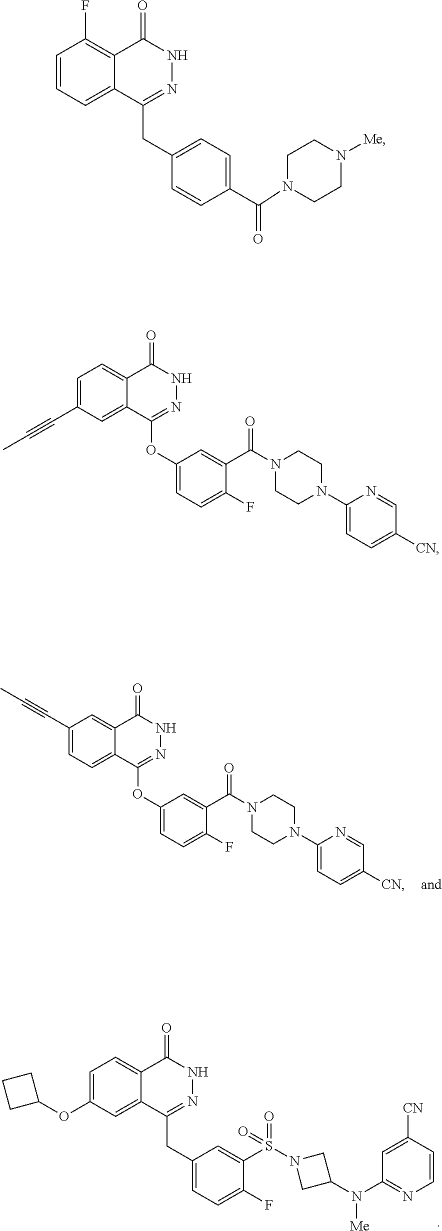 [SMILES: CN1CCN(C(=O)c2ccc(Cc3n[nH]c(=O)c4c(F)cccc34)cc2)CC1 ; CC#Cc1ccc2c(=O)[nH]nc(Oc3ccc(F)c(C(=O)N4CCN(c5ccc(C#N)cn5)CC4)c3)c2c1 ; CC#Cc1ccc2c(Oc3ccc(F)c(C(=O)N4CCN(c5ccc(C#N)cn5)CC4)c3)n[nH]c(=O)c2c1 ; CN(c1cc(C#N)ccn1)C1CN(S(=O)(=O)c2cc(Cc3n[nH]c(=O)c4ccc(OC5CCC5)cc34)ccc2F)C1]

Embodiment I-76. A compound, and stereoisomers and pharmaceutically acceptable salts thereof, selected from the group consisting of

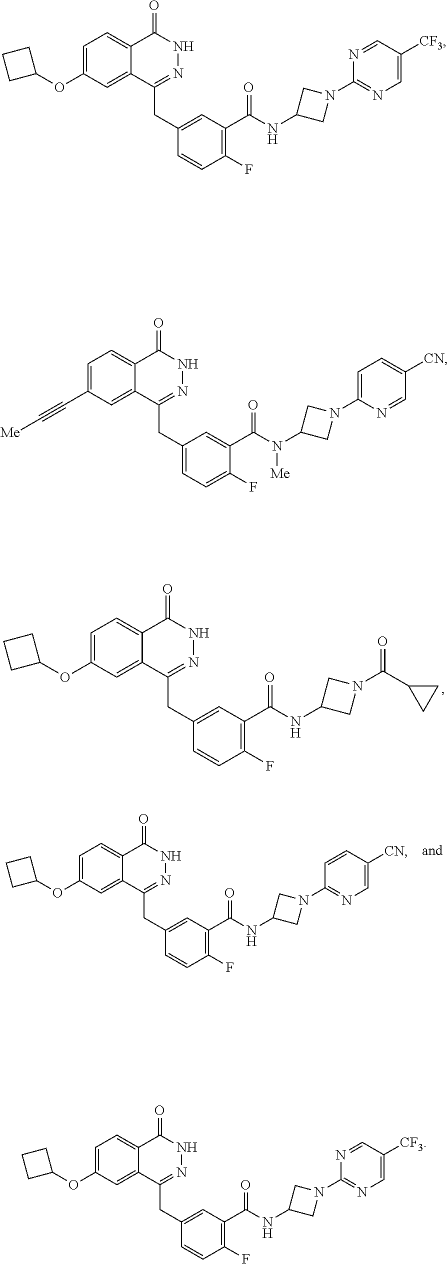 [SMILES: O=C(NC1CN(c2ncc(C(F)(F)F)cn2)C1)c1cc(Cc2n[nH]c(=O)c3ccc(OC4CCC4)cc23)ccc1F ; CC#Cc1ccc2c(=O)[nH]nc(Cc3ccc(F)c(C(=O)N(C)C4CN(c5ccc(C#N)cn5)C4)c3)c2c1 ; O=C(NC1CN(C(=O)C2CC2)C1)c1cc(Cc2n[nH]c(=O)c3ccc(OC4CCC4)cc23)ccc1F ; N#Cc1ccc(N2CC(NC(=O)c3cc(Cc4n[nH]c(=O)c5ccc(OC6CCC6)cc45)ccc3F)C2)nc1 ; O=C(NC1CN(c2ncc(C(F)(F)F)cn2)C1)c1cc(Cc2n[nH]c(=O)c3ccc(OC4CCC4)cc23)ccc1F]

Embodiment I-77. A compound, and stereoisomers and pharmaceutically acceptable salts thereof, selected from the group consisting of

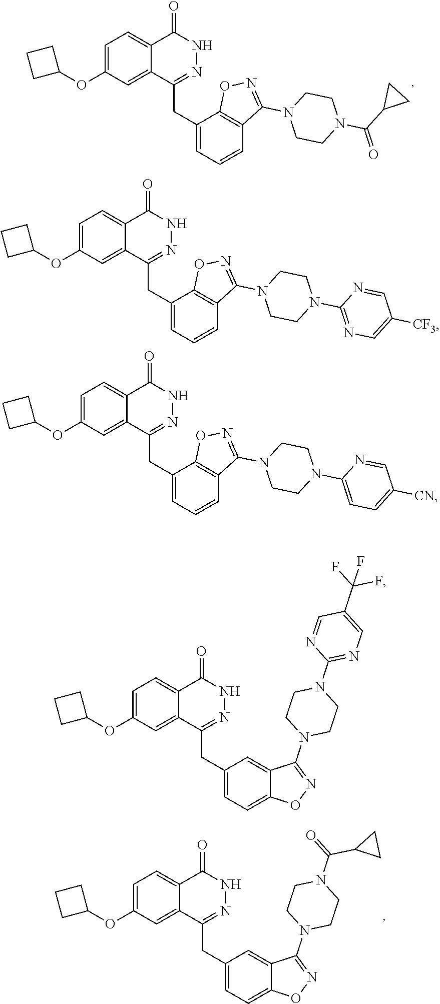

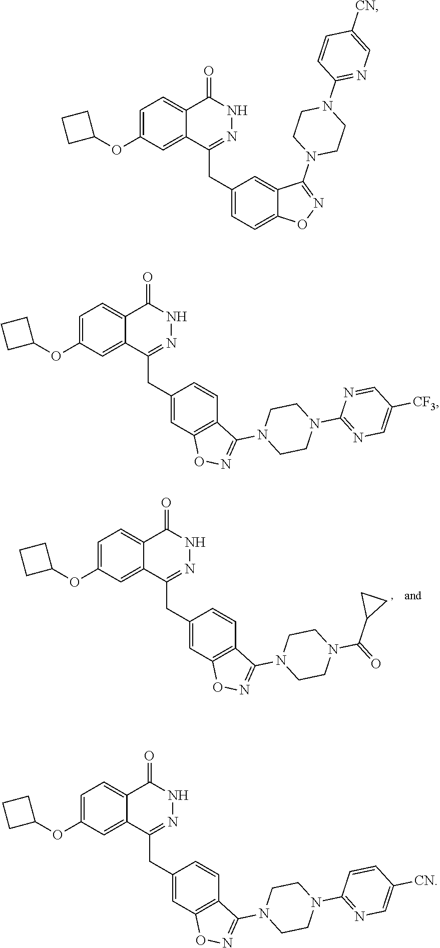 [SMILES: N#Cc1ccc(N2CCN(c3noc4ccc(Cc5n[nH]c(=O)c6ccc(OC7CCC7)cc56)cc34)CC2)nc1 ; O=c1[nH]nc(Cc2ccc3c(N4CCN(c5ncc(C(F)(F)F)cn5)CC4)noc3c2)c2cc(OC3CCC3)ccc12 ; O=C(C1CC1)N1CCN(c2noc3cc(Cc4n[nH]c(=O)c5ccc(OC6CCC6)cc45)ccc23)CC1 ; N#Cc1ccc(N2CCN(c3noc4cc(Cc5n[nH]c(=O)c6ccc(OC7CCC7)cc56)ccc34)CC2)nc1]

Embodiment I-78. A compound, and stereoisomers and pharmaceutically acceptable salts thereof, selected from the group consisting of

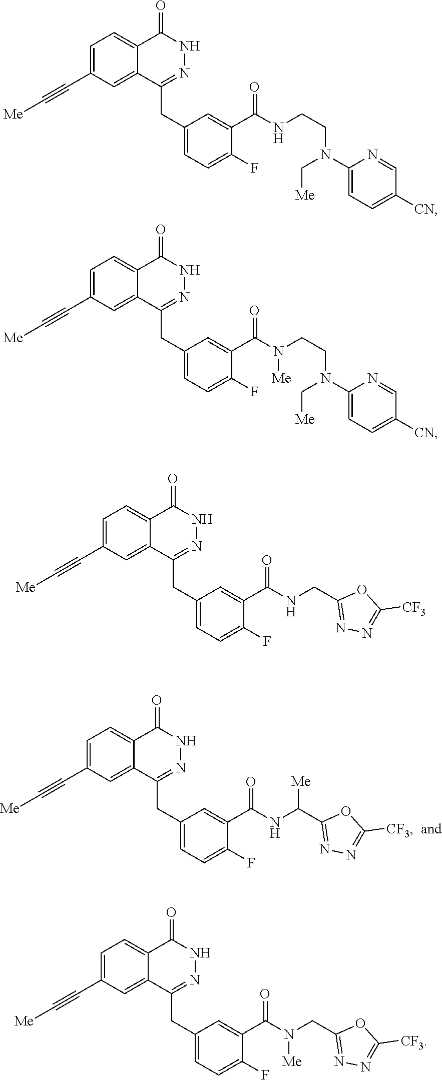

Embodiment I-79. The compound of any one of embodiments I-1 to I-78, wherein the compound selectively inhibits PARP7.

Embodiment I-80. A pharmaceutical composition comprising a compound of any one of embodiments I-1 to I-79, and stereoisomers and pharmaceutically acceptable salts thereof, and a pharmaceutically acceptable carrier.

Embodiment I-81. A compound of any one of embodiments I-1 to I-79, and stereoisomers and pharmaceutically acceptable salts thereof, for use in medicine.

Embodiment I-82. A compound of any one of embodiments I-1 to I-79, and stereoisomers and pharmaceutically acceptable salts thereof, for use in the treatment of a disorder that is responsive to inhibition of PARP7.

Embodiment I-83. Use of a compound of any one of embodiments I-1 to I-79, and stereoisomers and pharmaceutically acceptable salts thereof, in the treatment of a disorder that is responsive to inhibition of PARP7.

Embodiment I-84. A compound of any one of embodiments I-1 to I-79, and stereoisomers and pharmaceutically acceptable salts thereof, for use in the manufacture of a medicament for the treatment of a disorder that is responsive to inhibition of PARP7.

Embodiment I-85. A method of treating a disorder in a subject in need thereof, wherein the disorder is mediated by PARP7, comprising administering to the subject a compound of any one of embodiments I-1 to I-79, and stereoisomers and pharmaceutically acceptable salts thereof.

Embodiment I-86. The compound of embodiment I-82 or I-84, the use of embodiment I-83, or the method of embodiment I-85, wherein the disorder is selected from the group consisting of cancer, cardiovascular disorder, neurological disorder, inflammatory disorder, autoimmune disorder, and infectious disease.

Embodiment I-87. The compound of embodiment I-82, I-84, or I-86, the use of embodiment I-83 or I-86, or the method of embodiment I-85 or I-86, wherein the disorder is cancer and the cancer is of solid organ origin or of hematopoietic origin.

Embodiment I-88. The compound of embodiment I-82, I-84, I-86, or I-87 the use of embodiment I-83, I-86, or I-87 or the method of embodiment I-85, I-86, or I-87, wherein the disorder is cancer, the cancer is of solid organ origin, and the solid organ is selected from the group consisting of the brain, breast, colon, endometrium, esophagus, head and neck, upper gastrointestinal tract, respiratory tract, lung, kidney, liver, lower gastrointestinal tract, small intestine, large intestine, ovary, pancreas, prostate, stomach, testes, and urinary tract.

Embodiment I-89. The compound of embodiment I-82, I-84, I-86, or I-87 the use of embodiment I-83, I-86, or I-87 or the method of embodiment I-85, I-86, or I-87, wherein the disorder is cancer and the cancer is leukemia or lymphoma.

Embodiment I-90. The compound, use, or method of embodiment I-89, wherein the leukemia is acute lymphocytic leukemia (ALL), acute myelogenous leukemia (AML), chronic lymphocytic leukemia (CLL), or chronic myelogenous leukemia (CML).

Embodiment I-91. The compound, use, or method of embodiment I-89, wherein the lymphoma is Hodgkin's lymphoma, non-Hodgkin's lymphoma, multiple myeloma, B-cell lymphoma, diffuse large B-cell lymphoma (DLBCL), chronic lymphocytic lymphoma (CLL), T-cell lymphoma, hairy cell lymphoma, or Burkett's lymphoma.

Embodiment I-92. The compound of embodiment I-82, I-84, or I-86, the use of embodiment I-83 or I-86, or the method of embodiment I-85 or I-86, wherein the disorder is cancer and the cancer is selected from the group consisting of cancer of the bladder, bone cancer, cancer of the cervix, cancer of the epithelium, cancer of the gallbladder, cancer of the rectum, skin cancer, thyroid cancer, and cancer of the uterus.

### EXAMPLES

### General Synthetic Procedures

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

| | |
|---|---|
| c | acetyl |
| ACN | acetonitrile |
| APTB | 5-[di(1-adamantyl)phosphino]-12,32,52-triphenyl- |
| | 12H-[1,42]bipyrazole |
| aq. | aqueous |
| atm | atmospheres |
| Boc | tert-butoxy carbonyl |
| Boc₂O | Di-t-butyl dicarbonate |
| BrettPhos | 2-(Dicyclohexylphosphino)3,6-dimethoxy-2′,4′,6′- |
| | triisopropyl-1,1′-biphenyl |
| DAST | Diethylaminosulfur trifluoride |
| DCM | dichloromethane |
| DIPEA | N,N-Diisopropyl ethylamine |
| DMP; Dess-Martin | 1,1,1-Tris(acetyloxy)-1,1-dihydro-1,2- |
| | benziodoxol-3-(1H)-one |
| DMA | Dimethyl adipate |
| DMF | Dimethylformamide |
| DMSO | dimethylsulfoxide |
| eq(s). | equivalent(s) |
| EDCI | 1-Ethyl-3-(3-dimethylaminopropyl)carbodiimide |
| EtOAc/EA | ethyl acetate |
| Et | Ethyl |
| EtOH | ethanol |
| Et₃N | triethylamine |
| g | gram(s) |
| h | hour(s) |
| HATU | (Dimethylamino)-N,N-dimethyl(3H- |
| | [1,2,3]triazolo[4,5-6]pyridin-3- |
| | yloxy)methaniminium hexafluorophosphate |
| Hex | hexane |
| HOBt | 1-Hydroxybenzotriazole |
| HPLC | High pressure liquid chromatography |
| IPA | isopropanol |
| LCMS; LC-MS | liquid chromatography mass spectrometry |
| MeOH | methanol |
| mg | milligram(s) |
| min | Minute(s) |
| mL; ml | milliliter(s) |
| MS | mass spectrometry |
| mW | megawatt |
| NMe | N-methyl |
| NMP | N-Methyl-2-pyrrolidone |
| NMR | Nuclear magnetic resonance |
| Pd₂dba₃ | Tris(dibenzylideneacetone) dipalladium(0) |
| Ph | phenyl |
| r.t.; rt; RT | Room temperature |
| S.; sat. | saturated |
| TEA | triethylamine |
| TFA | trifluoroacetic acid |
| THF | tetrahydrofuran |
| TLC | Thin layer chromatography |
| Ts | Tosyl; 4-Methylphenylsulfonyl |
| X-Phos | 2-Dicyclohexylphosphino-2′,4′,6′- |
| | triisopropylbiphenyl |

Routine ¹H NMR spectra were recorded on 400, or 500 MHz spectrometers (Agilent, Oxford or Bruker) at ambient temperature, NMR solvents, d-chloroform (CDCl₃), d₆-dimethylsulfoxide (DMSO-d₆), and deuterated methanol (CD₃OD) were purchased from commercial suppliers and used without further purification. Spectra were processed using the automatic phasing and polynomial baseline correction features of the software. In cases where two adjacent peaks of equal or unequal height were observed, these two peaks may be labeled as either a multiplet or as a doublet. In the case of a doublet, a coupling constant using this software may be assigned. In any given example, one or more protons may not be observed due to obscurity by water and/or solvent peaks. Spectral data are reported as follows: chemical shift (multiplicity [singlet (s), broad singlet (bs), doublet (d), triplet (t), quartet (q), sextuplet (sex), multiplet (m), apparent (app), doublet of doublets (dd), doublet of doublet of doublets (ddd), doublet of triplets (dt)], coupling constant, integration). Chemical shifts are reported in ppm (δ), and coupling constants are reported in Hz. ¹H Resonances are referenced to solvent residual peaks for CDCl₃ (7.26 ppm), DMSO-d₆ (2.50 ppm), CD₃OD (3.30 ppm).

The HPLC-UV/MS instrumentation for product analysis consisted of a Waters Alliance 2695 with a column heater coupled with a ZQ 4000 mass spectrometer. The MS was equipped with an electrospray ionization (ESI) source and used in scan mode (100-1200 amu, source temperature: 150° C.) for both positive and negative ionization. The HPLC was equipped with a Waters photodiode array detector (PDA) 2998 (range used: 195-320 nm). The analytical method was developed on a XBridge C18 column (3.5 μm particle size, 4.6×30 mm) with a 10 mM buffer (ammonium formate pH 3.8 or ammonium bicarbonate pH 10)—A % and acetonitrile—B % as the mobile phase. A flow rate of 3 mL/min at 25° C. was set and the following gradient was used: 1) 5% B isocratic for 0.2 min, 5%-100% B in 1.8 min, 100% B for 1 min, or 2) 5% B isocratic for 0.2 min, 5%-100% B in 5.8 min, 100% B for 1 min.

In some other instances, HPLC-UV/MS instrumentation for product analysis consisted of a Agilent LCMS, Column: Waters X-Bridge C18 (50 mm×4.6 mm×3.5 μm); Column Temperature: 40° C.; Flow Rate: 1.5 mL/min; Mobile Phase: from 90% [water+10 mM NH₄HCO₃] and 5% [CH₃CN] to 0% [water+10 mM NH₄HCO₃] and 100% [CH₃CN] in 6.0 min, then under this condition for 2.0 min. finally changed to 90% [water+10 mM NH₄HCO₃] and 5% [CH₃CN] in 0.1 min and under this condition for 1 min.

### Prep-HPLC Conditions

An example of preparative HPLC condition employed to purify products is described below. Purifications were not limited to the gradient illustrated below and variations of the illustrated gradient were made according to polarity of the products obtained.

### 1.1 Chromatographic Equipment

Gilson Prep-HPLC system: GX-281 sample manager, 306 pump, 806 Manometric module, 811 D DYNAMIC Mixer, UV/VIS-156

### 1.2 Chromatographic Condition

Column: Waters X-Bridge™ Prep C18 5 μm OBD™, 19×250 mm
Flowrate: 20 mL/min
Gradient:

TABLE 1
Chromatographic gradient conditions

| | | Water |
|---|---|---|
| Time(min) | ACN | (10 mM NH₄HCO₃) |
| 0 | 10% | 90% |
| 1.0 | 10% | 90% |
| 3.95 | 45% | 55% |
| 19.10 | 60% | 40% |
| 19.35 | 95% | 5% |
| 24.50 | 95% | 5% |
| 24.80 | 10% | 90% |
| 30.80 | 10% | 90% |

Wavelength: 214 nm and 254 nm.

### EXAMPLES

The following synthetic examples are provided to illustrate the present disclosure, and should not be construed as limiting thereof. In these examples, all parts and percentages are by weight, unless otherwise noted.

### Synthesis of Example 1: 6-(4-(3-((7-Hydroxy-4-oxo-3,4-dihydrophthalazin-1yl)methyl)benzoyl)piperazin-1-yl)nicotinonitrile

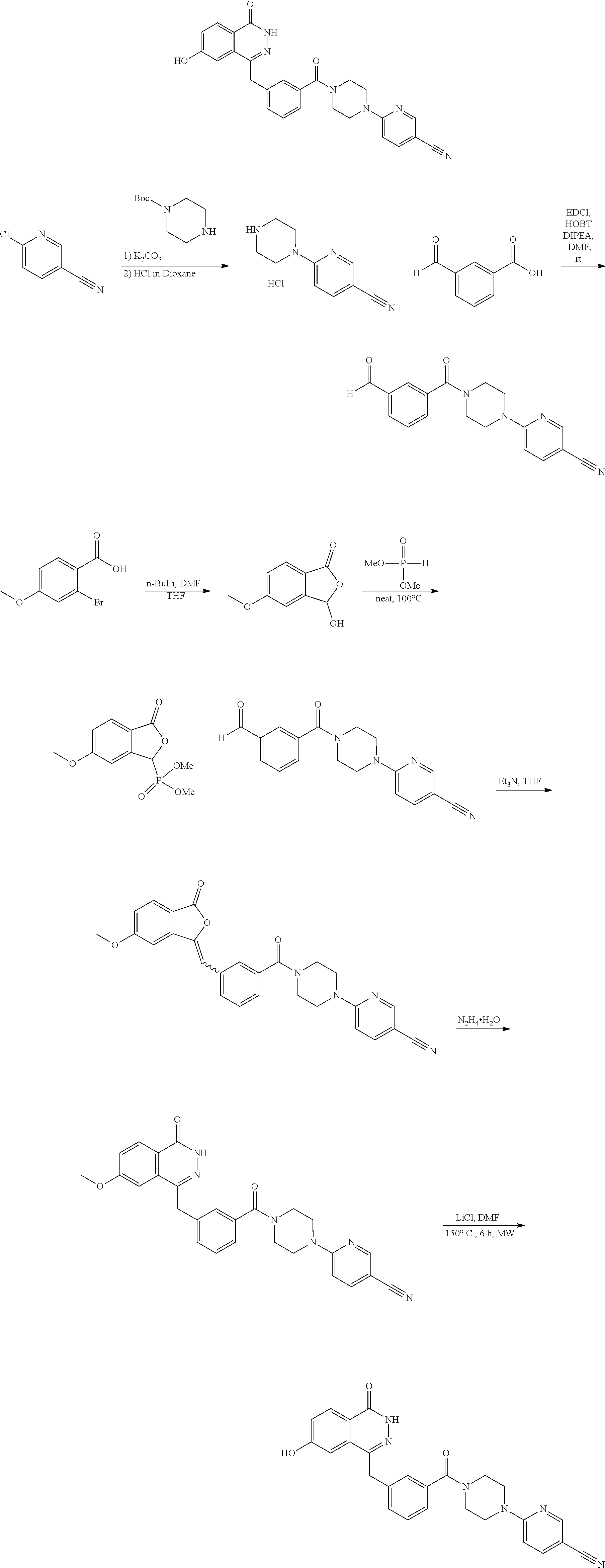

### Step 1:

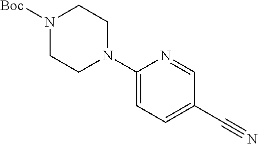 [SMILES: CC(C)(C)OC(=O)N1CCN(c2ccc(C#N)cn2)CC1]

### tert-Butyl 4-(5-cyanopyridin-2-yl)piperazine-1-carboxylate

A mixture of 6-chloronicotinonitrile (10.0 g, 72.2 mmol), tert-butyl piperazine-1-carboxylate (13.6 g, 72.9 mmol) and K₂CO₃ (17.0 g, 122.7 mmol) in MeCN (10 mL) was stirred at 60° C. for 12 hours. The mixture was cooled to rt, diluted with H₂O (50 mL) and extracted with EtOAc (50 mL×3). The combined organic layer was washed with brine, dried over anhydrous Na₂SO₄, filtered and concentrated in vacuo. The residue was purified by silica gel chromatography (EtOAc/pet ether=0 to 30%) to give tert-butyl 4-(5-cyanopyridin-2-yl)piperazine-1-carboxylate as a white solid (15.4 g, 73% yield). LCMS ESI m/z: 289 [M+H]⁺.

### Step 2:

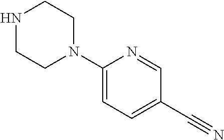 [SMILES: N#Cc1ccc(N2CCNCC2)nc1]

### 6-(Piperazin-1-yl)nicotinonitrile hydrochloride

A solution of tert-butyl 4-(5-cyanopyridin-2-yl)piperazine-1-carboxylate (15.4 g, 53.4 mmol) in HCl/dioxane (4M, 50 mL) was stirred at rt for 1 hour. The reaction mixture was concentrated in vacuo to give 6-(piperazin-1-yl)nicotinonitrile hydrochloride as a white solid (11.5 g, 96% yield). This was used without further purification. LCMS ESI m/z 189 [M+H]⁺.

### Step 3:

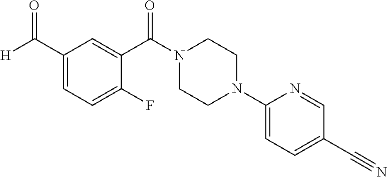 [SMILES: N#Cc1ccc(N2CCN(C(=O)c3cc(C=O)ccc3F)CC2)nc1]

### 6-(4-(2-Fluoro-5-formylbenzoyl)piperazin-1-yl)nicotinonitrile

To a solution of 2-fluoro-5-formylbenzoic acid (10.0 g, 59.5 mmol) and 6-(piperazin-1-yl)nicotinonitrile (10.2 g, 54.1 mmol) in DMF (100 mL) was added EDCI (12.1 g, 81.1 mmol), HOBt (11.0 g, 81.1 mmol) and DIPEA (16.3 g, 162 mmol), The reaction mixture was stirred at rt for 16 hours. The mixture was diluted with H₂O (100 mL) and extracted with EtOAc (100 mL×3). The combined organic layer was washed with brine, dried over anhydrous Na₂SO₄, filtered and concentrated in vacuo. The residue was purified by silica gel chromatography (EtOAc/pet ether=0 to 30%) to give 6-(4-(2-fluoro-5-formylbenzoyl)piperazin-1-yl)nicotinonitrile as a white solid (18 g, 94% yield). LCMS ESI m/z 339 [M+H]⁺.

### Step 4

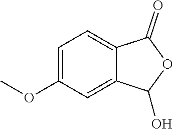

### 3-Hydroxy-5-methoxyisobenzofuran-1(3H)-one

To a stirred solution of 2-bromo-4-methoxybenzoic acid (5.00 g, 21.6 mmol) in anhydrous THF (5.17 mL) at −78° C. was added n-BuLi (2.77 g, 43.3 mmol) dropwise under argon. After stirred at this temperature for 0.5 hour, the mixture was treated with DMF (4.75 g, 64.9 mmol, 5.03 mL). The resulting solution was kept at −78° C. for 1 hour, then the reaction mixture was allowed to warm to ambient temperature. Water (10 mL) was added dropwise to the mixture, the aqueous layer was washed with diethyl ether (2×30 mL), shaken, and acidified with 2 M HCl until the solution reached pH 5˜6. The organic layer was separated, dried and concentrated to the residue. The residue was purified by silica gel chromatography (petroleum ether:EtOAc=10:1 to 3:1) to give 3-hydroxy-5-methoxy-3H-isobenzofuran-1-one (1.5 g, 38% yield). ¹H NMR (400 MHz, DMSO-d₆) δ 8.18 (d, J=6.8 Hz, 1H), 7.78 (d, J=9.0 Hz, 1H), 7.22 (d, J=5.9 Hz, 2H), 6.62 (d, J=6.8 Hz, 1H), 3.94 (s, 3H).

### Step 5

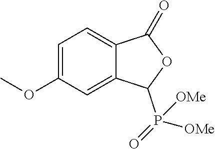 [SMILES: COc1ccc2c(c1)C(P(=O)(OC)OC)OC2=O]

### Dimethyl 6-methoxy-3-oxo-1,3-dihydroisobenzofuran-1-ylphosphonate

To a solution of 3-hydroxy-5-methoxy-3H-isobenzofuran-1-one (1.50 g, 8.33 mmol) in dimethylphosphite (15 mL) was stirred at 100° C. for 2 hours then cooled to room temperature. The mixture was poured into water (30 mL), extracted with ethyl acetate (40 mL) for twice. The combined organic layers were washed with brine, dried and concentrated to the crude. The crude was purified by silica gel chromatography (petroleum ether:EtOAc=30:1 to 1:1) to give dimethyl 6-methoxy-3-oxo-1,3-dihydroisobenzofuran-1-ylphosphonate (1.70 g, 75% yield). LCMS ESI m/z: 273.0 [M+H]⁺.

### Step 6

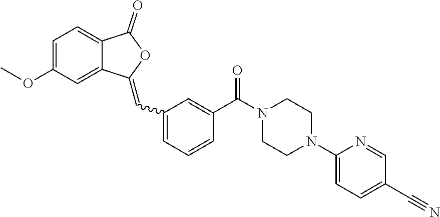 [SMILES: COc1ccc2c(c1)C(=Cc1cccc(C(=O)N3CCN(c4ccc(C#N)cn4)CC3)c1)OC2=O]

### 6-(4-(3-((6-Methoxy-3-oxoisobenzofuran-1(3H)-ylidene)methyl)benzoyl) piperazin-1-yl)nicotinonitrile

To a mixture of dimethyl 6-methoxy-3-oxo-1,3-dihydroisobenzofuran-1-ylphosphonate (800 mg, 2.94 mmol) in THF (20 mL) was added 6-[4-(3-formylbenzoyl)piperazin-1-yl]pyridine-3-carbonitrile (1.13 g, 3.53 mmol) and Et₃N (892 mg, 8.82 mmol, 1.23 mL). The resulting mixture was stirred at 70° C. for 10 hours. The mixture was quenched with sat. NaHSO₃ solution (50 mL) and filtered. The filter cake was washed with H₂O (20 mL×2), dried and concentrated under reduced pressure to give 6-(4-(3-((6-methoxy-3-oxoisobenzofuran-1(3H)-ylidene)methyl)benzoyl)piperazin-1-yl)nicotinonitrile (900 mg, 66% yield) as a white solid. LCMS ESI 467.2 [M+H]⁺.

### Step 7

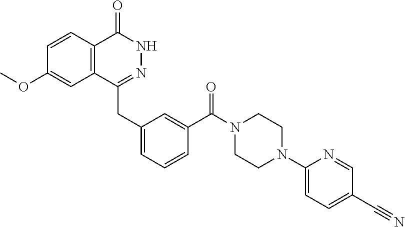 [SMILES: COc1ccc2c(=O)[nH]nc(Cc3cccc(C(=O)N4CCN(c5ccc(C#N)cn5)CC4)c3)c2c1]

### 6-(4-(3-((7-Methoxy-4-oxo-3,4-dihydrophthalazin-1-yl)methyl)benzoyl)piperazin-1-yl)nicotinonitrile

To a mixture of 6-(4-(34(6-methoxy-3-oxoisobenzofuran-1(3H)-ylidene)methyl)benzoyl)piperazin-1-yl)nicotinonitrile (759 mg, 1.57 mmol) in THF (9.26 mL) was added hydrazine hydrate (784 mg, 15.7 mmol, 764 μL). The resulting mixture was stirred at 70° C. for 3 hours. The mixture was cooled to rt, and filtered. The filter cake was washed with a solution of petroleum ether/EtOAc=10/1 (20 mL×2) and dried under reduced pressure to give 6-(4-(3-((7-methoxy-4-oxo-3,4-dihydrophthalazin-1-yl)methyl)benzoyl)piperazin-1-yl)nicotinonitrile (500 mg, 64% yield) as a white solid. LCMS ESI m/z 481.2 [M+H]⁺.

### Step 8

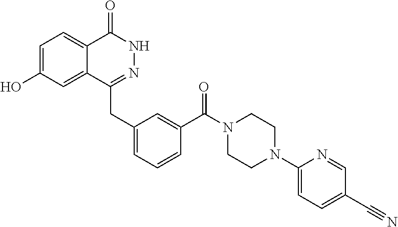 [SMILES: N#Cc1ccc(N2CCN(C(=O)c3cccc(Cc4n[nH]c(=O)c5ccc(O)cc45)c3)CC2)nc1]

### 6-(4-(3-((7-Hydroxy-4-oxo-3,4-dihydrophthalazin-1yl)methyl)benzoyl)piperazin-1-yl)nicotinonitrile

To a solution of 6-(4-(3-((7-methoxy-4-oxo-3,4-dihydrophthalazin-1-yl)methyl)benzoyl)piperazin-1-yl)nicotinonitrile (130 mg, 271 μmol) in DMF (2 mL) was added LiCl (34.4 mg, 812 μmol, 16.6 μL) at 25° C. The reaction was stirred at 150° C. for 6 hours under microwave. The mixture was filtered and purified by prep-HPLC to give 6-(4-(3-((7-hydroxy-4-oxo-3,4-dihydrophthalazin-1-yl)methyl)benzoyl)piperazin-1-yl)nicotinonitrile (51.6 mg, 41% yield) as a white solid. LCMS ESI 467.2 [M+H]⁺. ¹H-NMR (400 MHz, DMSO-d₆) δ 12.32 (s, 1H), 8.51 (d, J=2.0 Hz, 1H), 8.10 (d, J=8.7 Hz, 1H), 7.89 (d, J=9.1, 2.4 Hz, 1H), 7.43-7.37 (m, 2H), 7.35 (s, 1H), 7.32-7.26 (m, 1H), 7.22 (dd, J=8.7, 2.3 Hz, 1H), 7.11 (d, J=2.2 Hz, 1H), 6.90 (d, J=9.1 Hz, 1H), 4.25 (s, 2H), 3.68 (m, 8H).

### Synthesis of Example 2: 6-(4-(3-((4-Oxo-7-phenoxy-3,4-dihydrophthalazin-1-yl)methyl)benzoyl)piperazin-1-yl)nicotinonitrile

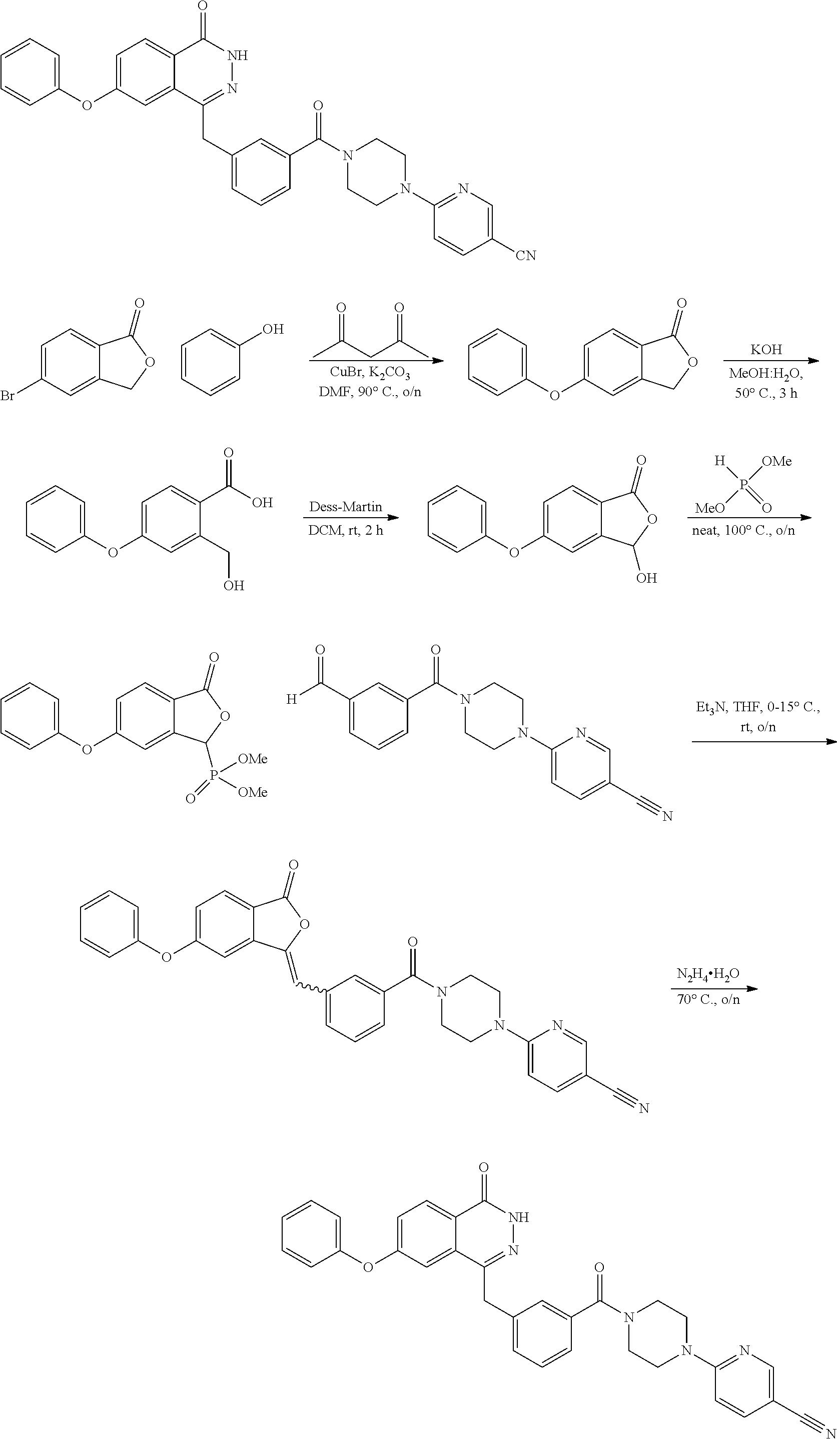

### Step 1

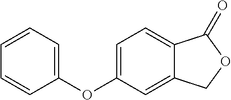 [SMILES: O=C1OCc2cc(Oc3ccccc3)ccc21]

### 5-Phenoxyisobenzofuran-1(3H)-one

To a mixture of 5-bromoisobenzofuran-1(3H)-one (5.0 g, 23 mmol) and phenol (4.3 g, 46 mmol) in dry DMF (100 mL) was successively added CuBr (0.33 g, 2.3 mmol), pentane-2,4-dione (0.46 g, 4.6 mmol) and K₂CO₃ (6.4 g, 46 mmol) at room temperature. The resulting mixture was flushed with N₂ for 10 min. Then the reaction was allowed to heat to 90° C. and stirred at this temperature for overnight. The slurry was filtered over a pad of celite. The filtrate was concentrated. The filtrate was diluted with H₂O (100 ml). The mixture was extracted with EtOAc (50 mL×3). The combined organic layers were washed with 1 N NaOH (40 mL×2). The organic layer was separated, dried, filtered and concentrated. The crude product was purified by silica gel chromatography (petroleum ether/EtOAc=30/1 to 10/1, v/v) to give 5-phenoxyisobenzofuran-1(3H)-one (2.0 g, 38% yield) as a light yellow solid. ¹H NMR (400 MHz, CDCl₃) δ 7.85 (d, J=8.0 Hz, 1H), 7.45-7.41 (m, 2H), 7.45-7.26 (m, 1H), 7.26-7.25 (m, 1H), 7.13-7.08 (m, 2H), 6.94 (d, J=4.0 Hz, 1H), 5.33 (d, J=4.0 Hz, 2H).

### Step 2

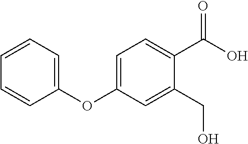 [SMILES: O=C(O)c1ccc(Oc2ccccc2)cc1CO]

### 2-(Hydroxymethyl)-4-phenoxybenzoic acid

To a solution of 5-phenoxy isobenzofuran-1(3H)-one (1.4 g, 6.2 mmol), MeOH (15 mL) and H₂O (15 mL) was added KOH (0.69 g, 12 mmol) at room temperature. The resulting mixture was allowed to warm to 50° C. and stirred at the same temperature for 3 hours. The volatile was removed. The residue was dissolved in H₂O (40 mL). The solution was acidified with 1 N HCl until the solution reached pH 2-3. The suspension was filtered, and the solid was collected. The product was dried in vacuo to give 2-(hydroxymethyl)-4-phenoxybenzoic acid (1.3 g, 87%) as a white solid, which was directly used for the next step without further purification. ¹H NMR (400 MHz, CDCl₃) δ 7.91 (d, J=8.0 Hz, 1H), 7.47-7.45 (m, 2H), 7.30-7.29 (m, 1H), 7.25-7.23 (m, 1H), 7.12-7.10 (m, 2H), 6.90-6.87 (m, 1H), 4.82 (d, J=8.0 Hz, 2H).

### Step 3

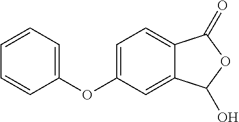 [SMILES: O=C1OC(O)c2cc(Oc3ccccc3)ccc21]

### 3-Hydroxy-5-phenoxyisobenzofuran-1(3H)-one

To a slurry of 2-(hydroxymethyl)-4-phenoxy benzoic acid (1.1 g, 4.5 mmol) in dry DCM (30 mL) was added Dess-Martin periodinane (2.3 g, 5.4 mmol) in portionwise at 0° C. Then the reaction was allowed to warm to room temperature, and stirred at the same temperature for 2 hours. The suspension was filtered, and the solid was rinsed with DCM (50 mL). The filtrate was washed by a sat. aq of Na₂SO₃ (30 mL×2). The organic layer was separated, dried, filtered and concentrated. The residue was purified by silica gel chromatography (DCM/MeOH=20/1 to 10/1, v/v) to give 3-hydroxy-5-phenoxyisobenzofuran-1(3H)-one (0.6 g, 55% yield) as a white solid. ¹H NMR (400 MHz, CDCl₃) δ 7.81 (d, J=8.0 Hz, 1H), 7.44-7.43 (m, 2H), 7.28-7.23 (m, 2H), 7.17 (dd, J=4.0 Hz, J=8.0 Hz, 1H), 7.11-7.08 (m, 3H), 6.52 (s, 1H).

### Step 4

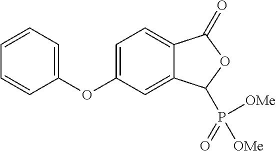 [SMILES: COP(=O)(OC)C1OC(=O)c2ccc(Oc3ccccc3)cc21]

### Dimethyl 3-oxo-6-phenoxy-1,3-dihydroisobenzofuran-1-ylphosphonate

A flask were charged 3-hydroxy-5-phenoxyisobenzofuran-1(3H)-one (0.6 g, 2.5 mmol) and dimethyl phosphonate (10 mL) under N₂. The mixture was heated to 100° C. and stirred at the same temperature for overnight. The reaction was diluted with EtOAc (50 mL). The solution was washed by water (15 mL×7). The organic layer was separated, dried, filtered and concentrated. The residue was purified by silica gel chromatography (petroleum ether/EtOAc=1/1, v/v) to give dimethyl 3-oxo-6-phenoxy-1,3-dihydroisobenzofuran-1-ylphosphonate (0.15 g, 18% yield) as a yellow solid.

### Step 5

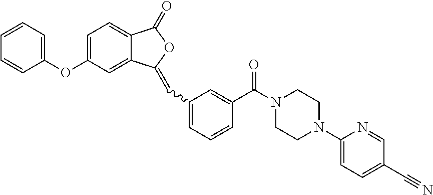 [SMILES: N#Cc1ccc(N2CCN(C(=O)c3cccc(C=C4OC(=O)c5ccc(Oc6ccccc6)cc54)c3)CC2)nc1]

### 6-(4-(3-((3-Oxo-6-phenoxyisobenzofuran-1(3H)-ylidene)methyl)benzoyl)piperazin-1-yl)nicotinonitrile

To a mixture of dimethyl 3-oxo-6-phenoxy-1,3-<dihydroisobenzofuran-1-ylphosphonate (0.15 g, 0.45 mmol) and 6-(4-(3-formylbenzoyl)piperazin-1-yl)nicotinonitrile (0.43 g, 1.3 mmol) in dry THF (5 mL) was added Et₃N (91 mg, 0.9 mmol) dropwise at 0° C. under N₂. The reaction mixture was allowed to warm to room temperature, and stirred at the same temperature for overnight. The mixture was poured into a mixture of DCM (50 mL) and a sat. aq of NaHSO₃ (50 mL). The solution was vigorously stirred at room temperature for 1 hour. The organic layer was separated, dried, filtered and concentrated. The residue was purified by silica gel chromatography (petroleum ether/EtOAc=5/1 to 1/1, v/v) to give 6-(4-(3-((3-oxo-6-phenoxyisobenzofuran-1(3H)-ylidene)methyl)benzoyl)piperazin-1-yl)nicotinonitrile (0.2 g, 83% yield) as a white solid. LCMS ESI m/z: 529.2 [M+H]⁺.

### Step 6

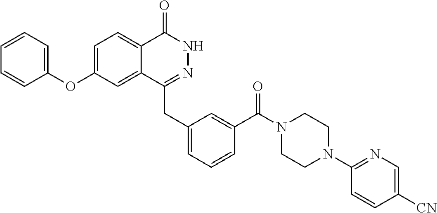 [SMILES: N#Cc1ccc(N2CCN(C(=O)c3cccc(Cc4n[nH]c(=O)c5ccc(Oc6ccccc6)cc45)c3)CC2)nc1]

### 6-(4-(3-((4-Oxo-7-phenoxy-3,4-dihydrophthalazin-1-yl)methyl)benzoyl)piperazin-1-yl)nicotinonitrile

A mixture of 6-(4-(3-((3-oxo-6-phenoxyisobenzofuran-1(3H)-ylidene)methyl)benzoyl)piperazin-1-yl)nicotinonitrile (0.22 g, 0.44 mmol) and N₂H₄·H₂O (0.8 mL) in THF (1.6 mL) was stirred at 70° C. for 2 hours in a sealed tube. The slurry was filtered, and the solid was collected. The product was triturated in MTBE (20 mL) for overnight. The suspension was filtered. The product was dried in vacuo to give 6-(4-(3-((4-oxo-7-phenoxy-3,4-dihydrophthalazin-1-yl)methyl)benzoyl)piperazin-1-yl)nicotinonitrile (0.11 g, 49% yield) as a white solid. LCMS ESI m/z: 543.2 [M+H]⁺. ¹H NMR (400 MHz, DMSO-d₆) δ 12.56 (s, 1H), 8.49 (d, J=4.0 Hz, 1H), 8.26 (d, J=8.0 Hz, 1H), 7.87 (dd, J=8.0, 4.0 Hz, 1H), 7.46 (t, J=8.0 Hz, 2H), 7.38-7.37 (m, 3H), 7.30-7.28 (m, 4H), 7.09 (d, J=8.0 Hz, 2H), 6.87 (d, J=12.0 Hz, 1H), 4.25 (s, 2H), 3.76-3.62 (m, 6H), 3.41-3.40 (m, 2H).

### Synthesis of Example 3: 6-(4-(2-Fluor-((4-oxo-7-(prop-2-yn-1-yloxy)-3,4-dihydrophthalazin-1yl)methyl)benzoyl) piperazin-1-yl)nicotinonitrile

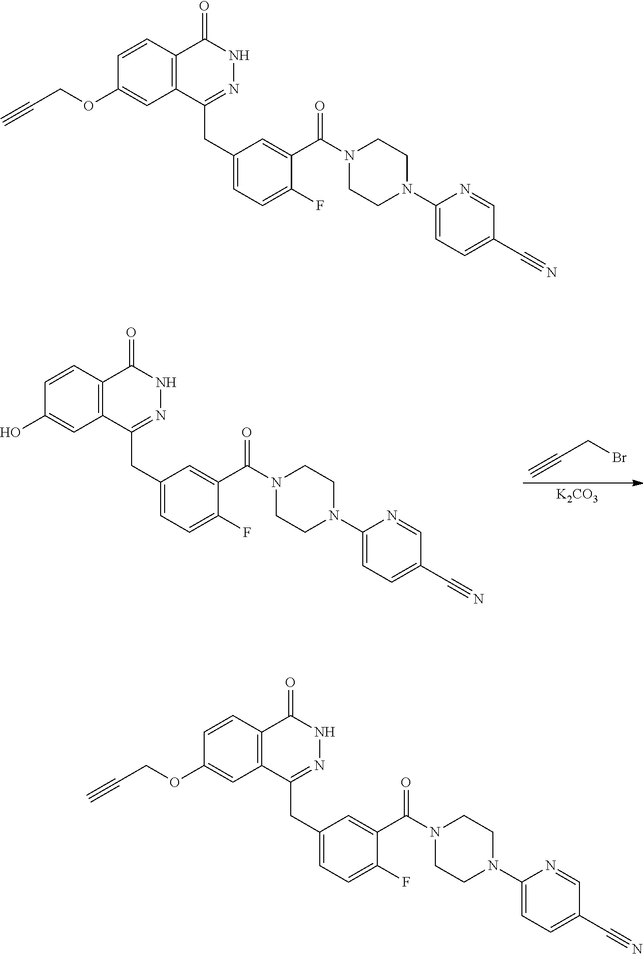

### Step 1:

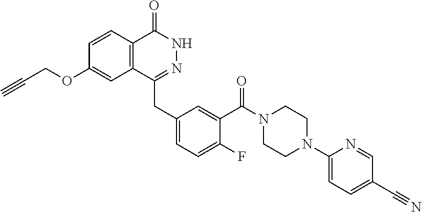 [SMILES: C#CCOc1ccc2c(=O)[nH]nc(Cc3ccc(F)c(C(=O)N4CCN(c5ccc(C#N)cn5)CC4)c3)c2c1]

### 6-(4-(2-Fluoro-5-((4-oxo-7-(prop-2-yn-1-yloxy)-3,4-dihydrophthalazin-yl)methyl)benzoyl) piperazin-1-yl)nicotinonitrile

A mixture of 6-(4-(3-((7-hydroxy-4-oxo-3,4-dihydrophthalazin-1yl)methyl)benzoyl)piperazin-1-yl)nicotinonitrile (100 mg, 0.21 mmol), 3-bromoprop-1-yne (26 mg, 0.22 mmol) and K₂CO₃ (86 mg, 0.63 mmol) in DMF (5 mL) was stirred at 50° C. for 1 hour. The mixture was cooled to rt, diluted with H₂O (10 mL) and extracted with EtOAc (20 mL×3). The combined organic layer was concentrated in vacuo and the residue was purified by prep-HPLC to give 6-(4-(2-fluoro-5-((4-oxo-7-(prop-2-yn-1-yloxy)-3,4-dihydrophthalazin-1-yl)methyl) benzoyl)piperazin-1-yl)nicotinonitrile as a white solid (40 mg, 37% yield). LCMS ESI m/z: 523 [M+H]⁺. ¹H NMR (400 MHz, CDCl₃) δ 9.95 (s, 1H), 8.34 (dd, J=10.0, 5.5 Hz, 2H), 7.59 (dd, J=9.0, 2.3 Hz, 1H), 7.29 (dd, J=12.8, 4.1 Hz, 3H), 7.14 (d, J=2.4 Hz, 1H), 7.00 (t, J=8.7 Hz, 1H), 6.55 (d, J=9.0 Hz, 1H), 4.71 (d, J=2.4 Hz, 2H), 4.18 (s, 2H), 3.82 (s, 2H), 3.71 (s, 2H), 3.62 (t, J=5.1 Hz, 2H), 3.36 (s, 2H), 2.58 (t, J=2.4 Hz, 1H).

### Synthesis of Example 4: 6-(4-(3-((7-Methoxy-4-oxo-3,4-dihydrophthalazin-1-yl)methyl)benzoyl)piperazin-1-yl)nicotinonitrile

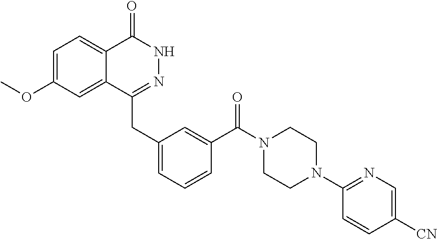 [SMILES: COc1ccc2c(=O)[nH]nc(Cc3cccc(C(=O)N4CCN(c5ccc(C#N)cn5)CC4)c3)c2c1]

Following the procedure described in Example 1 and making non-critical variations as required to replace 2-(hydroxymethyl)-4-phenoxybenzoic acid with 2-(hydroxymethyl)-4-methoxybenzoic acid, 6-(4-(3-((7-methoxy-4-oxo-3,4-dihydrophthalazin-1-yl)methyl)benzoyl)piperazin-1-yl)nicotinonitrile was obtained as a white solid. LCMS ESI m/z: 481.1 [M+H]⁺. ¹H NMR (400 MHz, DMSO-d₆) δ 12.45 (s, 1H), 8.52 (d, J=2.0 Hz, 1H), 8.19 (d, J=8.8 Hz, 1H), 7.90 (dd, J=9.1, 2.4 Hz, 1H), 7.41 (m, 4H), 7.30 (t, J=5.4 Hz, 2H), 6.92 (d, J=9.1 Hz, 1H), 4.35 (s, 2H), 3.88 (s, 3H), 3.69 (s, 8H).

### Synthesis of Example 5: 6-(4-(3-((7-Chloro-4-oxo-3,4-dihydrophthalazin-1-yl)methyl)benzoyl)piperazin-1-yl)nicotinonitrile

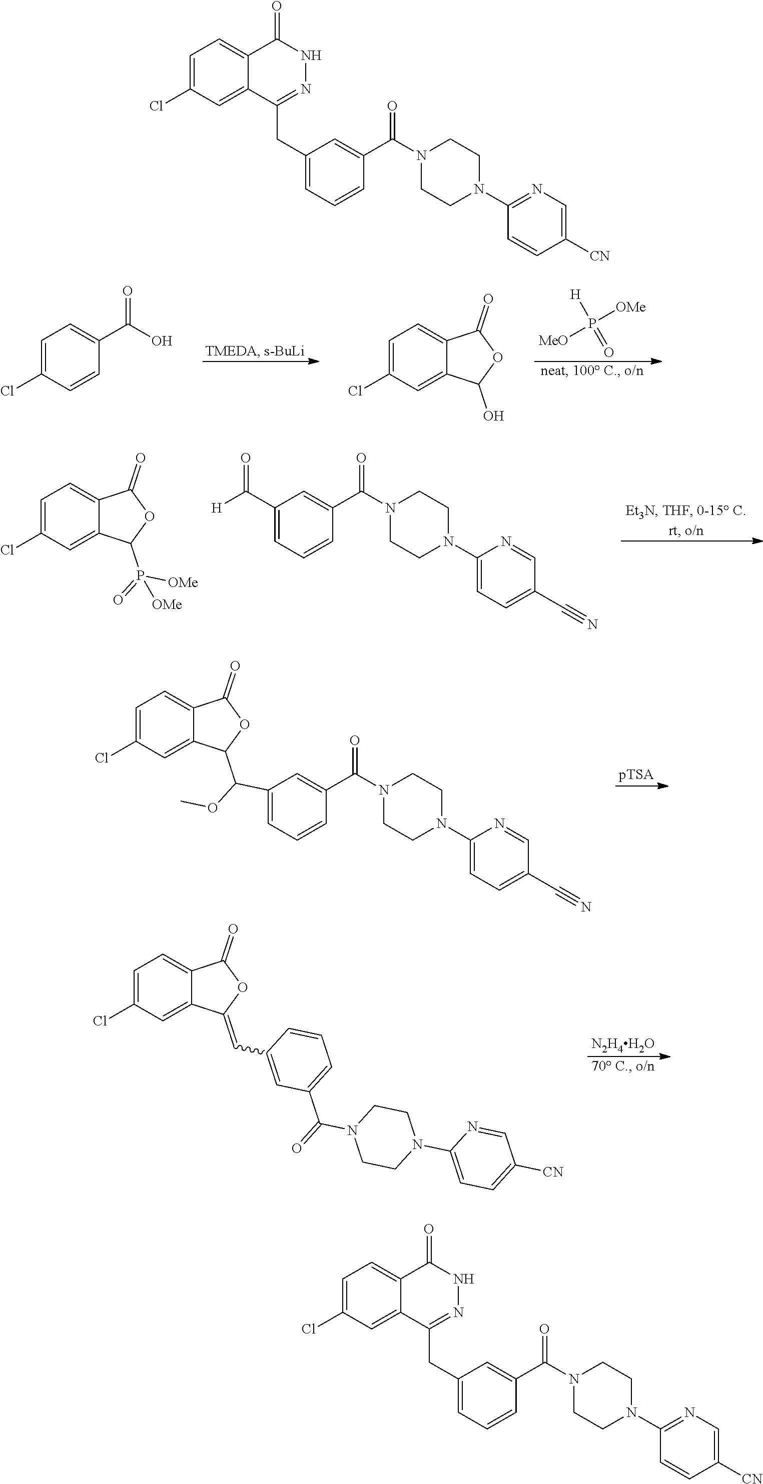

### Step 1

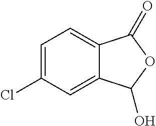 [SMILES: O=C1OC(O)c2cc(Cl)ccc21]

### 5-Chloro-3-hydroxyisobenzofuran-1(3H)-one

To a mixture of 4-chlorobenzoic acid (1.0 g, 6.4 mmol) in THF (10 mL) was added TMEDA (1.6 g, 14.1 mmol), The mixture was stirred at −78° C. for 2 minutes. Then s-BuLi (14.1 mL, 14.1 mmol, 1 M in THF) was drop-wise into the mixture, which was stirred at −78° C. for another 30 min. DMF (2 mL) was added to the mixture, and the mixture was stirred at −78° C. for another 1 h. The mixture was quenched with 4 N aq. HCl, extracted with EtOAc (30 mL), dried over Na₂SO₄ and concentrated to give a residue. The residue was purified by silica gel chromatography (petroleum ether/EtOAc=10/1 to 5/1, v/v) to give 5-chloro-3-hydroxyisobenzofuran-1(3H)-one (330 mg, 28% yield) as a white solid. ¹H NMR (400 MHz, CDCl₃) δ 8.64 (d, J=2.0 Hz, 1H), 8.24 (dd, J=8.4, 2.1 Hz, 1H), 7.84 (s, 1H), 7.66 (s, 1H), 6.61 (s, 1H).

### Step 2

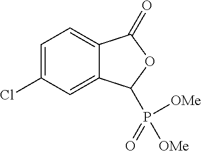 [SMILES: COP(=O)(OC)C1OC(=O)c2ccc(Cl)cc21]

### Dimethyl 6-chloro-3-oxo-1,3-dihydroisobenzofuran-1-ylphosphonate

Following the procedure of step 5 in Example 1, but starting with 5-chloro-3-hydroxyisobenzofuran-1(3H)-one gave the title compound as a colorless oil. ¹H NMR (400 MHz, CDCl₃) δ 7.88 (d, J=8.2 Hz, 1H), 7.75 (s, 1H), 7.57 (d, J=8.2 Hz, 1H), 5.67 (m, 1H), 3.94 (m, 3H), 3.75-3.55 (m, 3H).

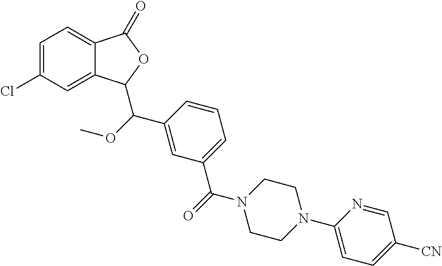 [SMILES: COC(c1cccc(C(=O)N2CCN(c3ccc(C#N)cn3)CC2)c1)C1OC(=O)c2ccc(Cl)cc21]

### 6-(4-(3-((6-Chloro-3-oxo-1,3-dihydroisobenzofuran-1yl)(methoxy)methyl)benzoyl) piperazin-1-yl)nicotinonitrile

To a solution of dimethyl 6-chloro-3-oxo-1,3-dihydroisobenzofuran-1-ylphosphonate (100 mg, 0.4 mmol) in THF (2 mL) was added 6-(4-(3-formylbenzoyl)piperazin-1-yl)nicotinonitrile (366 mg, 1.1 mmol), Then Et₃N (116 mg, 1.1 mmol) was drop-wise into the mixture, which was stirred at 20° C. for 16 hrs. The solids were collected by filtration, washed with petroleum ether (5 mL×2) and dried under vacuum to give 6-(4-(3-((6-chloro-3-oxo-1,3-dihydroisobenzofuran-1-yl)(methoxy)methyl)benzoyl)piperazin-1-yl)nicotinonitrile (150 mg, 84% yield) as a yellow solid. LCMS ESI m/z: 503.1 [M+H]⁺.

### Step 4

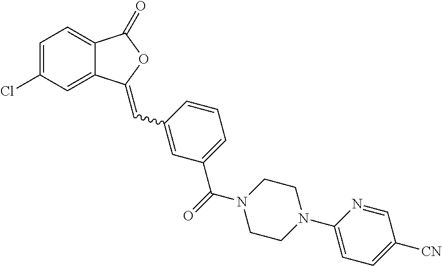 [SMILES: N#Cc1ccc(N2CCN(C(=O)c3cccc(C=C4OC(=O)c5ccc(Cl)cc54)c3)CC2)nc1]

### 6-(4-(3-((6-Chloro-3-oxoisobenzofuran-1(3H)-ylidene)methyl)benzoyl)piperazin-1-yl)nicotinonitrile

To a mixture of 6-(4-(3-((6-chloro-3-oxo-1,3-dihydroisobenzofuran-1-yl)(methoxy)methyl)benzoyl)piperazin-1-yl)nicotinonitrile (130 mg, 0.3 mmol) in toluene (5 mL) was added PTSA (5 mg, 0.03 mmol), The resulting mixture was stirred at 100° C. for 1 hr. The mixture was concentrated to give the crude 6-(4-(3-((6-chloro-3-oxoisobenzofuran-1(3H)-ylidene)methyl)benzoyl)piperazin-1-yl)nicotinonitrile (110 mg, 90%/o yield) as yellow gum. LCMS ESI m/z: 571.1 [M+H]⁺.

### Step 5

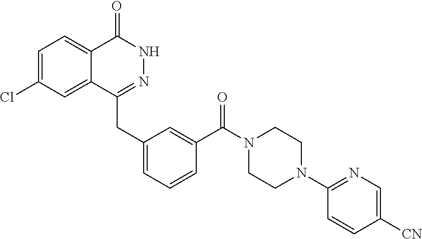 [SMILES: N#Cc1ccc(N2CCN(C(=O)c3cccc(Cc4n[nH]c(=O)c5ccc(Cl)cc45)c3)CC2)nc1]

### 6-(4-(3-((7-Chloro-4-oxo-3,4-dihydrophthalazin-1-yl)methyl)benzoyl)piperazin-1-yl)nicotinonitrile

Following the procedure of step 7 in Example 1, but starting with 6-(4-(3-((6-chloro-3-oxoisobenzofuran-1(3H)-ylidene)methyl)benzoyl)piperazin-1-yl)nicotinonitrile gave the title compound as a white solid. LCMS ESI m/z: 484.7 [M+H]⁺. ¹H NMR (400 MHz, DMSO-d₆) δ 12.64 (s, 1H), 8.44 (d, J=1.9 Hz, 1H), 8.18 (d, J=8.5 Hz, 1H), 7.97 (d, J=1.9 Hz, 1H), 7.85-7.77 (m, 2H), 7.37-7.31 (m, 3H), 7.26-7.20 (m, 1H), 6.84 (m, 1H), 4.30 (s, 2H), 3.61 (s, 6H), 3.32 (d, J=6.8 Hz, 2H).

### Synthesis of Example 6: 6-(4-(3-((7-Fluoro-4-oxo-3,4-dihydrophthalazin-1-yl)methyl)benzoyl)piperazin-1-yl)nicotinonitrile

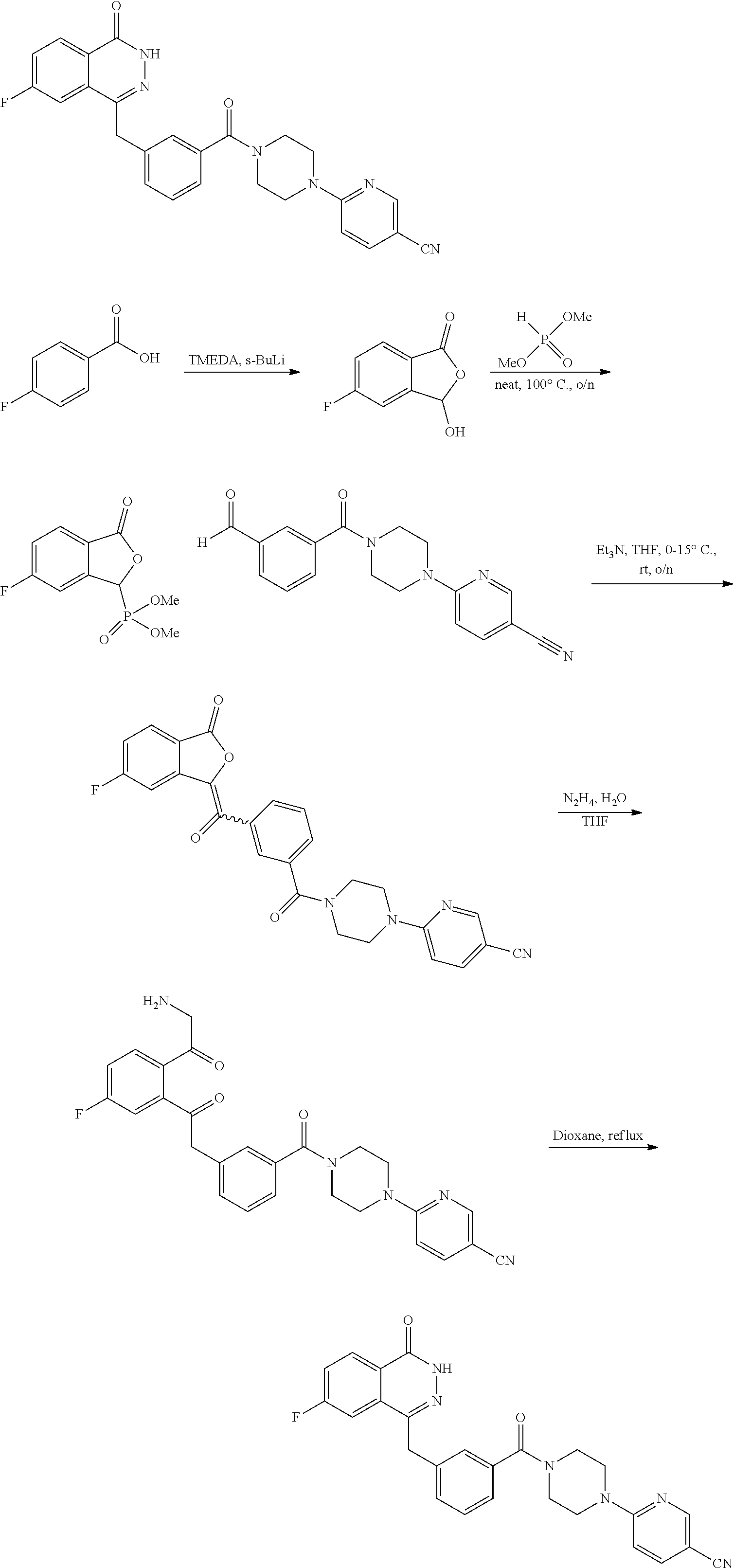

### Step 1

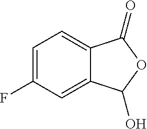 [SMILES: O=C1OC(O)c2cc(F)ccc21]

### 5-Fluoro-3-hydroxyisobenzofuran-1(3H)-one

Following the procedure of step 4 in Example 1, but starting with 4-fluorobenzoic acid gave the title compound as a yellow solid. LCMS ESI m/z: 169.1 [M+H]⁺.

### Step 2

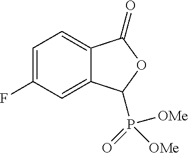 [SMILES: COP(=O)(OC)C1OC(=O)c2ccc(F)cc21]

### Dimethyl 6-fluoro-3-oxo-1,3-dihydroisobenzofuran-1-ylphosphonate

Following the procedure of step 5 in Example 1, but starting with 5-fluoro-3-hydroxyisobenzofuran-1(3H)-one gave the title compound as a yellow oil. LCMS ESI m/z: 261.1 [M+H]⁺.

### Step 3

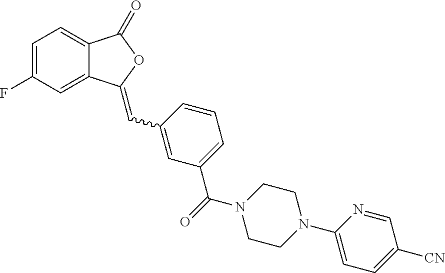 [SMILES: N#Cc1ccc(N2CCN(C(=O)c3cccc(C=C4OC(=O)c5ccc(F)cc54)c3)CC2)nc1]

### 6-(4-(3-((6-Fluoro-3-oxoisobenzofuran-1(3H)-ylidene)methyl)benzoyl)piperazin-1-yl)nicotinonitrile

Following the procedure of step 6 in Example 1, but starting with dimethyl 6-fluoro-3-oxo-1,3-dihydroisobenzofuran-1-ylphosphonate gave the title compound as a yellow solid. LCMS ESI m/z: 454.1 [M+H]⁺.

### Step 4

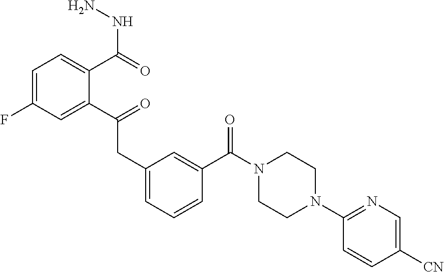 [SMILES: N#Cc1ccc(N2CCN(C(=O)c3cccc(CC(=O)c4cc(F)ccc4C(=O)NN)c3)CC2)nc1]

### 2-(2-(3-(4-(5-Cyanopyridin-2-yl)piperazine-1-carbonyl)phenyl)acetyl)-4-fluorobenzohydrazide

To a mixture of 6-(4-(3-((6-fluoro-3-oxoisobenzofuran-1(3H)-ylidene)methyl)benzoyl)piperazin-1-yl)nicotinonitrile (0.14 g, 0.64 mmol) and N₂H₄·H₂O (0.8 mL) in THF (1.6 mL) was stirred at 25° C. for 16 hours in a sealed tube. The reaction mixture was concentrated to give the crude product 2-(2-(3-(4-(5-cyanopyridin-2-yl)piperazine-1-carbonyl)phenyl)acetyl)-4-fluorobenzohydrazide as a yellow solid. LCMS ESI m/z: 487.1 [M+H]⁺.

### Step 5

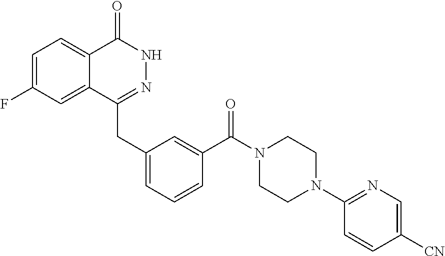 [SMILES: N#Cc1ccc(N2CCN(C(=O)c3cccc(Cc4n[nH]c(=O)c5ccc(F)cc45)c3)CC2)nc1]

### 6-(4-(3-((7-Fluoro-4-oxo-3,4-dihydrophthalazin-1-yl)methyl)benzoyl)piperazin-1-yl)nicotinonitrile

A mixture of 2-(2-(3-(4-(5-cyanopyridin-2-yl)piperazine-1-carbonyl)phenyl)acetyl)-4-fluorobenzohydrazide (0.012 g, 0.02 mmol) in dioxane (5 mL) was stirred at 100° C. for 5 hours in a sealed tube. The reaction mixture was concentrated to the crude. The residue was purified by Prep-TLC (DCM/MeOH=10/1) to give 6-(4-(3-((7-fluoro-4-oxo-3,4-dihydrophthalazin-1-yl)methyl)benzoyl)piperazin-1-yl)nicotinonitrile (8 mg, 70% yield) as a white solid. LCMS ESI m/z: 468.7 [M+H]⁺. ¹H NMR (400 MHz, DMSO-d₆) δ 12.68 (s, 1H), 8.52 (m, 1H), 8.34 (m, 1H), 7.91 (m, 1H), 7.89 (m, 1H), 7.70 (m, 1H), 7.43 (m, 3H), 7.31 (m, 1H), 6.91 (m, 1H), 4.31 (s, 2H), 3.69 (m, 8H).

### Synthesis of Example 7: 6-(4-(5-((7-(Cyclopropylmethoxy)-4-oxo-3,4-dihydrophthalazin-1-yl)methyl)-2-fluorobenzoyl)piperazin-1-yl)nicotinonitrile

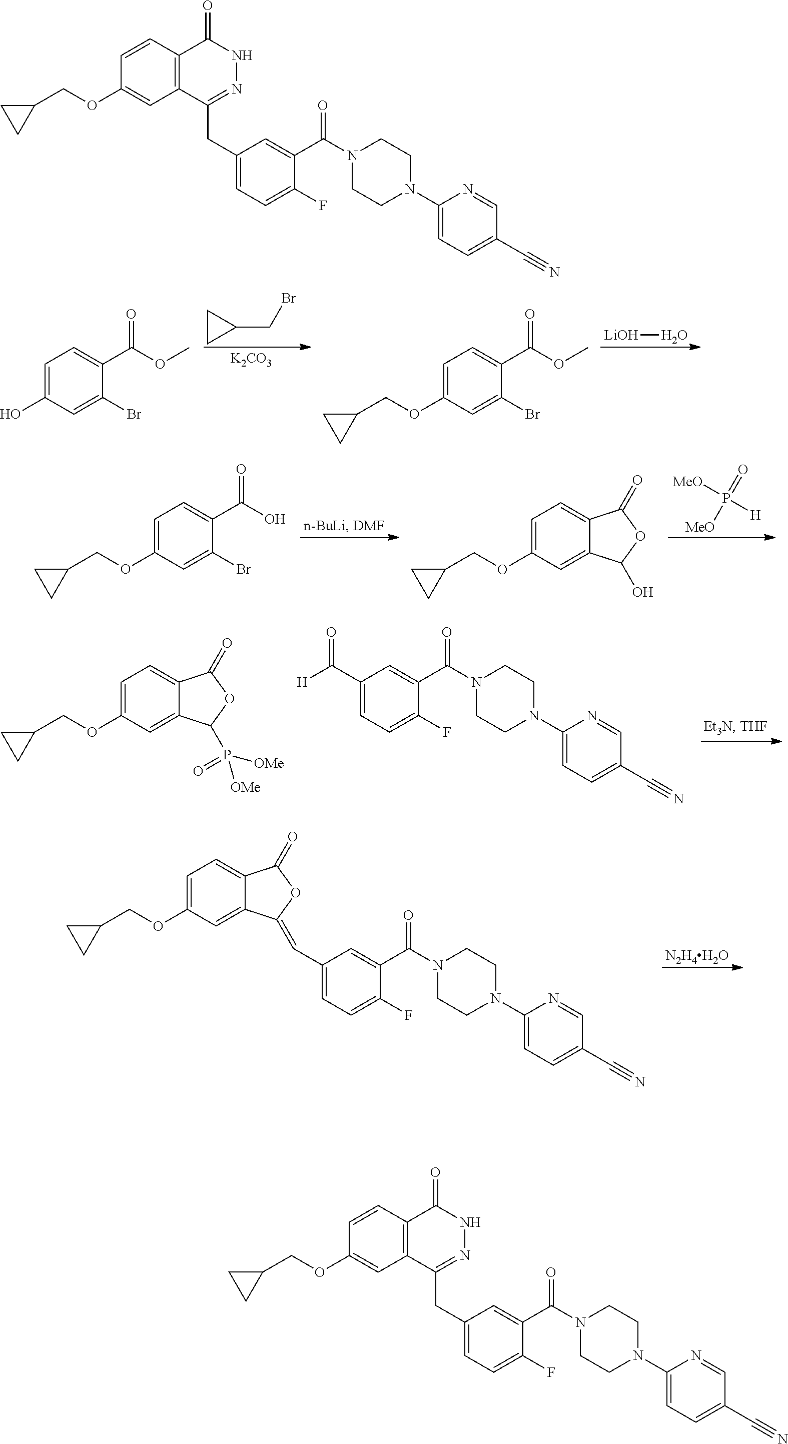

### Step 1

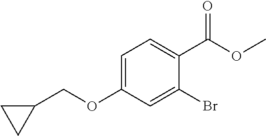 [SMILES: COC(=O)c1ccc(OCC2CC2)cc1Br]

### Methyl 2-bromo-4-(cyclopropylmethoxy)benzoate

A mixture of methyl 2-bromo-4-hydroxybenzoate (1.00 g, 4.33 mmol), (bromomethyl)cyclopropane (701 mg, 5.19 mmol) and K₂CO₃ (1.79 g, 12.98 mmol) in DMF (10 mL) was stirred at 65° C. under Ar (g) for 12 hours. The mixture was cooled to rt, diluted with H₂O (20 mL) and extracted with EtOAc (30 mL×3). The combined organic layer was washed with brine, dried over anhydrous Na₂SO₄, filtered and concentrated in vacuo to give methyl 2-bromo-4-(cyclopropylmethoxy)benzoate as a yellow oil (1.23 g, 98% yield). This was used without further purification. LCMS ESI m/z: 285 [M+H]⁺.

### Step 2

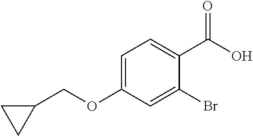 [SMILES: O=C(O)c1ccc(OCC2CC2)cc1Br]

### 2-Bromo-4-(cyclopropylmethoxy)benzoic acid

A mixture of methyl 2-bromo-4-(cyclopropylmethoxy)benzoate (1.23 g, 4.31 mmol) and LiOH—H₂O (362 mg, 8.62 mmol) in MeOH (12 mL) and H₂O (4 mL) was stirred at 50° C. for 12 hours. The organic solvent was removed in vacuo and aq HCl (2N) was added until the solution reached pH 5˜6, then extracted with EtOAc (30 mL×3). The combined organic layer was washed with brine, dried over anhydrous Na₂SO₄, filtered and concentrated in vacuo to give 2-bromo-4-(cyclopropylmethoxy)benzoic acid as a white solid (1.1 g, 94%). This was used without further purification. LCMS ESI m/z: 271 [M+H]⁺.

### Step 3

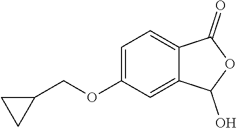 [SMILES: O=C1OC(O)c2cc(OCC3CC3)ccc21]

### 5-(Cyclopropylmethoxy)-3-hydroxyisobenzofuran-1(3H)-one

A solution of 2-bromo-4-(cyclopropylmethoxy)benzoic acid (900 mg, 3.32 mmol) in THF (10 mL) was cooled to −78° C. then n-BuLi (2.5 M in heptane, 2.66 mL, 6.64 mmol) was added dropwise under Ar (g). The reaction was stirred at −78° C. for 15 min, then DMF (533 mg, 7.30 mmol) was added. The reaction was stirred at −78° C. for 20 min, then quenched with sat. NH₄Cl solution (10 mL), added aq HCl (1N) until the solution reached pH 5˜6. The mixture was extracted with EtOAc (3×30 mL). The combined organic layer was washed with bine, dried over Na₂SO₄, filtered and concentrated in vacuo. The residue was purified by silica gel chromatography (EtOAc/pet ether=30 to 50%) to give 5-(cyclopropylmethoxy)-3-hydroxyisobenzofuran-1(3H)-one (110 mg, 14% yield). LCMS ESI m/z 221 (M+H)⁺.

### Step 4

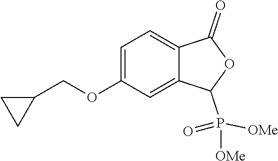 [SMILES: COP(=O)(OC)C1OC(=O)c2ccc(OCC3CC3)cc21]

### Dimethyl (6-(cyclopropylmethoxy)-3-oxo-1,3-dihydroisobenzofuran-1-yl)phosphonate

A solution of 5-(cyclopropylmethoxy)-3-hydroxyisobenzofuran-1(3H)-one (110 mg, 0.50 mmol) in dimethyl phosphonate (5 mL) was stirred at 100° C. for 3 hours. The mixture was cooled to rt, diluted with H₂O (10 mL) and extracted with EtOAc (20 mL×3). The combined organic layer was washed with brine, dried over anhydrous Na₂SO₄, filtered and concentrated in vacuo. The residue was purified by silica gel chromatography (EtOAc/petroleum ether=40 to 6(0%) to give dimethyl (6-(cyclopropylmethoxy)-3-oxo-1,3-dihydroisobenzofuran-1-yl)phosphonate as a yellow oil (135 mg, 87% yield). LCMS ESI m/z: 313 [M+H]⁺.

### Step 5

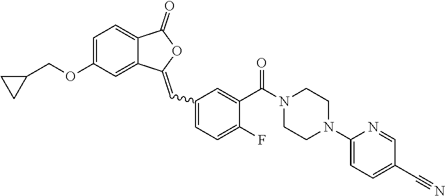 [SMILES: N#Cc1ccc(N2CCN(C(=O)c3cc(C=C4OC(=O)c5ccc(OCC6CC6)cc54)ccc3F)CC2)nc1]

### 6-(4-(5-((6-(Cyclopropylmethoxy)-3-oxoisobenzofuran-1(3H)-ylidene)methyl)-2-fluorobenzoyl)piperazin-1-yl)nicotinonitrile

A mixture of dimethyl (6-(cyclopropylmethoxy)-3-oxo-1,3-dihydroisobenzofuran-1-yl)phosphonate (110 mg, 0.35 mmol), 6-(4-(2-fluoro-5-formylbenzoyl)piperazin-1-yl)nicotinonitrile (143 mg, 0.42 mmol) and Et₃N (107 mg, 1.06 mmol) in THF (5 mL) was stirred at 60° C. for 3 hours. The mixture was cooled to rt, diluted with H₂O (30 mL) and extracted with DCM (30 mL×3). The combined organic layer was dried over anhydrous Na₂SO₄, filtered and concentrated in vacuo to give 6-(4-(5-((6-(cyclopropylmethoxy)-3-oxoisobenzofuran-1(3H)-ylidene)methyl)-2-fluorobenzoyl)piperazin-1-yl)nicotinonitrile as a white solid (180 mg, 97% yield). This was used without further purification. LCMS ESI m/z: 525 [M+H]⁺).

### Step 6

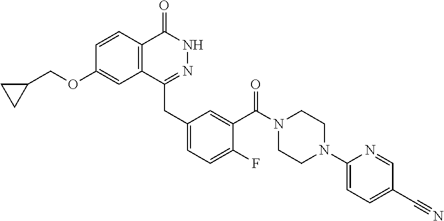 [SMILES: N#Cc1ccc(N2CCN(C(=O)c3cc(Cc4n[nH]c(=O)c5ccc(OCC6CC6)cc45)ccc3F)CC2)nc1]

### 6-(4-(5-((7-(Cyclopropylmethoxy)-4-oxo-3,4-dihydrophthalazin-1-yl)methyl)-2-fluorobenzoyl)piperazin-1-yl)nicotinonitrile

To a solution of 6-(4-(5-((6-(cyclopropylmethoxy)-3-oxoisobenzofuran-1(3H)-ylidene)methyl)-2-fluorobenzoyl)piperazin-1-yl)nicotinonitrile (180 mg, 0.34 mmol) in THF (5 mL) was added N₂H₄·H₂O (136 mg, 6.8 mmol) and heated to 60° C. for 3 hours. The solids were collected by filtration, washed with water and dried under vacuum to give (4-(5-((7-(cyclopropylmethoxy)-4-oxo-3,4-dihydrophthalazin-1-yl)methyl)-2-fluorobenzoyl)piperazin-1-yl)nicotinonitrile as a white solid (24 mg, 13% yield). LCMS ESI m/z 539 [M+H]⁺). ¹H NMR (400 MHz, DMSO-d₆) δ 12.44 (s, 1H), 8.51 (d, J=2.2 Hz, 1H), 8.16 (d, J=8.8 Hz, 1H), 7.90 (dd, J=9.1, 2.3 Hz, 1H), 7.48-7.36 (m, 3H), 7.26 (dd, J=14.9, 5.7 Hz, 2H), 6.93 (d, J=9.1 Hz, 1H), 4.31 (s, 2H), 3.95 (d, J=7.0 Hz, 2H), 3.82-3.68 (m, 4H), 3.67-3.57 (m, 2H), 3.32-3.29 (m, 2H), 1.29-1.14 (m, 1H), 0.63-0.49 (m, 2H), 0.41-0.27 (m, 2H).

### Synthesis of Example 8: 6-(4-(5-((7-(But-3-yn-2-yloxy)-4-oxo-3,4-dihydrophthalazin-1-yl)methyl)-2-fluorobenzoyl)piperazin-1-yl)nicotinonitrile

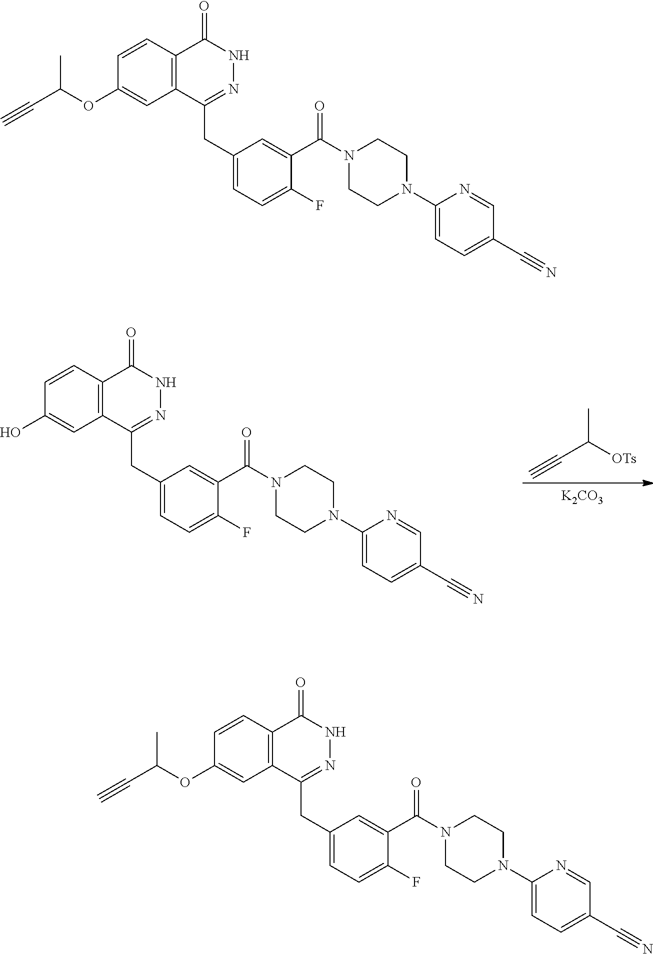

### Step 1

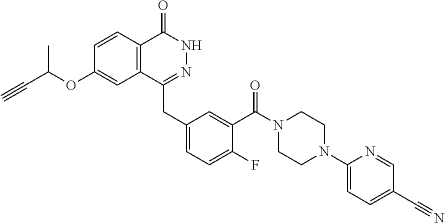 [SMILES: C#CC(C)Oc1ccc2c(=O)[nH]nc(Cc3ccc(F)c(C(=O)N4CCN(c5ccc(C#N)cn5)CC4)c3)c2c1]

### 6-(4-(5-((7-(But-3-yn-2-yloxy)-4-oxo-3,4-dihydrophthalazin-1-yl)methyl)-2-fluorobenzoyl)piperazin-1-yl)nicotinonitrile

A mixture of 6-(4-(2-fluoro-5-((7-hydroxy-4-oxo-3,4-dihydrophthalazin-1-yl)methyl)benzoyl)piperazin-1-yl)nicotinonitrile (40 mg, 0.08 mmol), but-3-yn-2-yl 4-methylbenzenesulfonate (27 mg, 0.12 mmol) and K₂CO₃ (33 mg, 0.24 mmol) in DMF (5 mL) was stirred at 50° C. for 1 hour. The solid were filtered off, and the filtrate was concentrated in vacuo. The residue was purified by prep-HPLC to give the desired product 6-(4-(5-((7-(but-3-yn-2-yloxy)-4-oxo-3,4-dihydrophthalazin-1-yl)methyl)-2-fluorobenzoyl)piperazin-1-yl)nicotinonitrile as a white solid (29 mg, 68% yield). LCMS ESI m/z 537 (M+H)⁺). ¹H-NMR (400 MHz, DMSO-d₆) δ 12.49 (s, 1H), 8.20 (d, J=8.8 Hz, 1H), 7.90 (dd, J=9.2, 2.4 Hz, 1H), 7.49-7.37 (m, 4H), 7.26 (t, J=9.2 Hz, 1H), 6.92 (d, J=8.8 Hz, 1H), 5.37-5.35 (m, 1H), 4.38-4.21 (m, 2H), 3.77-3.73 (m, 4H), 3.62-3.54 (m, 3H), 3.32-3.29 (m, 2H), 1.58 (d, J=6.8 Hz, 3H).

### Synthesis of Example 9: 6-(4-(2-Fluoro-5-((4-oxo-7-(2,2,2-trifluoroethoxy)-3,4-dihydrophthalazin-1-yl)methyl)benzoyl)piperazin-1-yl)nicotinonitrile

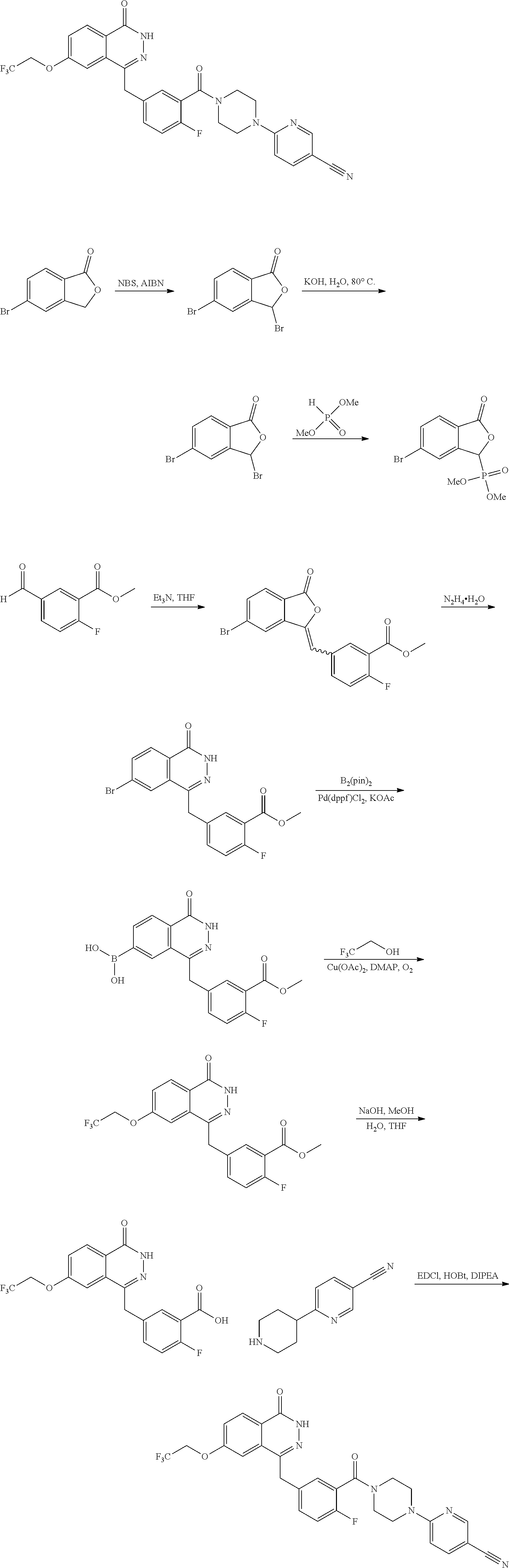

### Step 1

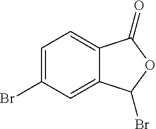 [SMILES: O=C1OC(Br)c2cc(Br)ccc21]

### 3,5-Dibromoisobenzofuran-1(3H)-one

A mixture of 5-bromoisobenzofuran-1(3H)-one (13.5 g, 63.4 mmol), NBS (13.5 g, 76.1 mmol) and AIBN (1.04 g, 6.34 mmol) in CCl₄ (100 mL) was heated to 80° C. and stirred for 16 hours. The mixture was cooled to rt, diluted with H₂O (30 mL) and extracted with DCM (80 mL×3). The combined organic layer was dried over anhydrous Na₂SO₄, filtered and concentrated in vacuo. The residue was purified by silica gel chromatography (EtOAc/petrole ether=0 to 15%) to afford 3,5-dibromoisobenzofuran-1(3H)-one as a light yellow solid (8.8 g, 48% yield). LCMS ESI m/z 291 [M+H]⁺.

### Step 2

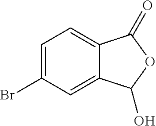 [SMILES: O=C1OC(O)c2cc(Br)ccc21]

### 5-(Difluoromethoxy)-3-hydroxyisobenzofuran-1(3H)-one

A mixture of 3,5-dibromoisobenzofuran-1(3H)-one (8.8 g, 16.6 mmol) and KOH (2.79 g, 49.7 mmol) in H₂O (25 mL) was stirred at 80° C. for 3 hours. The reaction was cooled to rt and aq HCl (1N) was added until the solution reached pH 5˜6. The solids were collected by filtration, washed with water and dried under vacuum to give 5-bromo-3-hydroxyisobenzofuran-1(3H)-one as a light yellow solid (6.4 g, 92% yield). This was used without further purification. LCMS ESI m/z 229 [M+H]⁺.

### Step 3

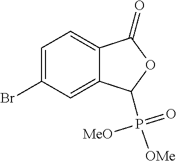 [SMILES: COP(=O)(OC)C1OC(=O)c2ccc(Br)cc21]

### Dimethyl (6-bromo-3-oxo-1,3-dihydroisobenzofuran-1-yl)phosphonate

A solution of 5-bromo-3-hydroxyisobenzofuran-1(3H)-one (6.4 g, 22.35 mmol) in dimethyl phosphonate (50 mL) was heated to 100° C. and stirred for 2 hours. The mixture was cooled to rt, diluted with H₂O (100 mL) and extracted with EtOAc (50 mL×3). The combined organic layer was washed with brine, dried over anhydrous Na₂SO₄, filtered and concentrated in vacuo. The residue was purified by silica gel chromatography (EtOAc/petroleum ether=30 to 50%) to afford dimethyl (6-bromo-3-oxo-1,3-dihydroisobenzofuran-1-yl)phosphonate as a white solid (5.68 g, 70%). LCMS ESI m-z 321 [M+H]⁺.

### Step 4

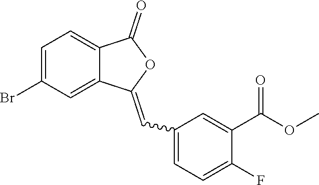 [SMILES: COC(=O)c1cc(C=C2OC(=O)c3ccc(Br)cc32)ccc1F]

### Methyl-5-((6-bromo-3-oxoisobenzofuran-1(3H)-ylidene)methyl)-2-fluorobenzoate

A solution of dimethyl (6-bromo-3-oxo-1,3-dihydroisobenzofuran-1-yl)phosphonate (5.00 g, 15.6 mmol), methyl 2-fluoro-5-formylbenzoate (3.12 g, 17.13 mmol) and Et₃N (4.73 g, 46.72 mmol) in THF (50 mL) was stirred at rt under Ar (g) for 12 hours. The reaction mixture was diluted with water (50 mL) and extracted with EtOAc (50 mL×3). The combined organic layer was washed with brine, dried over Na₂SO₄, filtered and concentrated in vacuo to afford methyl 5-((6-bromo-3-oxoisobenzofuran-1(3H)-ylidene)methyl)-2-fluorobenzoate as a white solid (5.01 g, 85% yield). This was used without further purification. LCMS ESI m/z 377 (M+H)⁺.

### Step 5

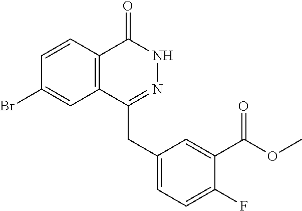 [SMILES: COC(=O)c1cc(Cc2n[nH]c(=O)c3ccc(Br)cc23)ccc1F]

### Methyl 5-((7-bromo-4-oxo-3,4-dihydrophthalazin-1-yl)methyl)-2-fluorobenzoate

A solution of methyl 5-((6-bromo-3-oxoisobenzofuran-1(3H)-ylidene)methyl)-2-fluorobenzoate (5.80 g, 13.3 mmol) and N₂H₄·H₂O (1.33 g, 26.5 mmol) in THF (50 mL) was stirred at 70° C. for 4 hours. The solids were filtered and washed with water (30 mL) to afford methyl 5-((7-bromo-4-oxo-3,4-dihydrophthalazin-1-yl)methyl)-2-fluorobenzoate as a white solid (4.1 g, 79% yield). This was used without further purification. LCMS ESI m/z 391 (M+H)⁺.

### Step 6

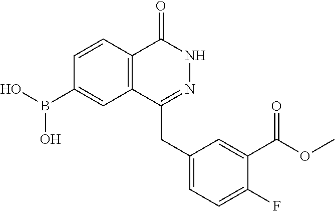 [SMILES: COC(=O)c1cc(Cc2n[nH]c(=O)c3ccc(B(O)O)cc23)ccc1F]

### (4-(4-Fluoro-3-(methoxycarbonyl)benzyl)-1-oxo-1,2-dihydrophthalazin-6-yl)boronic acid

A mixture of methyl 5-((7-bromo-4-oxo-3,4-dihydrophthalazin-1-yl)methyl)-2-fluorobenzoate (529 mg, 1.11 mmol), 4,4,4′,4′,5,5,5′,5′-octamethyl-2,2′-bi(1,3,2-dioxaborolane) (1.41 g, 5.54 mmol), KOAc (653 mg, 6.65 mmol) and Pd(dppf)Cl₂ (49 mg, 0.06 mmol) in DMSO (10 mL) was stirred at 80° C. under Ar (g) for 2 hours. The mixture was cooled to rt, diluted with H₂O (10 mL) and extracted with EtOAc (20 mL×3). The combined organic layer was washed with brine, dried over anhydrous Na₂SO₄, filtered and concentrated in vacuo to afford (4-(4-fluoro-3-(methoxycarbonyl)benzyl)-1-oxo-1,2-dihydrophthalazin-6-yl)boronic acid as a light yellow solid (520 mg, 67% yield). This was used without further purification. LCMS ESI m/z 357 (M+H)⁺.

### Step 7

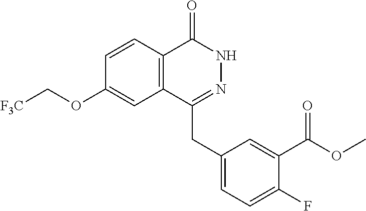 [SMILES: COC(=O)c1cc(Cc2n[nH]c(=O)c3ccc(OCC(F)(F)F)cc23)ccc1F]

### Methyl 2-fluoro-5-((4-oxo-7-(2,2,2-trifluoroethoxy)-3,4-dihydrophthalazin-1-yl)methyl)benzoate

A mixture of (4-(4-fluoro-3-(methoxycarbonyl)benzyl)-1-oxo-1,2-dihydrophthalazin-6-yl)boronic acid (60 mg, 0.17 mmol), Cu(OAc)₂·H₂O (4 mg, 0.02 mmol) and DMAP (41 mg, 0.33 mmol) in CF₃CH₂OH (1 mL) was stirred at 40° C. under O₂(g) for 12 hours. The reaction was quenched with water (10 mL) and extracted with EtOAc (10 mL×3). The combined organic layer was washed with brine, dried over anhydrous Na₂SO₄, filtered and concentrated in vacuo. The residue was purified by silica gel chromatography (EtOAc/petroleum ether=50 to 70%) to give methyl 2-fluoro-5-((4-oxo-7-(2,2,2-trifluoroethoxy)-3,4-dihydrophthalazin-1-yl)methyl)benzoate as a white solid (25 mg, 27% yield). LCMS ESI m/z 411[M+H]⁺.

### Step 8

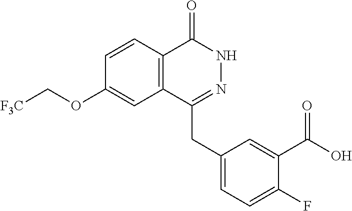 [SMILES: O=C(O)c1cc(Cc2n[nH]c(=O)c3ccc(OCC(F)(F)F)cc23)ccc1F]

### 2-Fluoro-5-((4-oxo-7-(2,2,2-trifluoroethoxy)-3,4-dihydrophthalazin-1-yl)methyl)benzoic acid

A mixture of methyl 2-fluoro-5-((4-oxo-7-(2,2,2-trifluoroethoxy)-3,4-dihydrophthalazin-1-yl)methyl)benzoate (25 mg, 0.06 mmol) and NaOH (24 mg, 0.6 mmol) in MeOH (i mL). THF (1 mL) and H₂O (1 mL) was stirred at rt for 1 hour. The organic solvent was removed in vacuo and aq HCl (1N) was added to until the solution reached pH 5˜6. The solids were collected by filtration, washed with water and dried under vacuum to give 2-fluoro-5-((4-oxo-7-(2,2,2-trifluoroethoxy)-3,4-dihydrophthalazin-1-yl)methyl)benzoic acid as a white solid (20 mg, 83% yield). LCMS ESI m/z 397 [M+H]⁺.

### Step 9

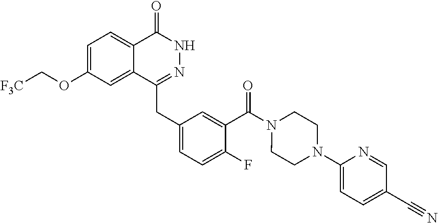 [SMILES: N#Cc1ccc(N2CCN(C(=O)c3cc(Cc4n[nH]c(=O)c5ccc(OCC(F)(F)F)cc45)ccc3F)CC2)nc1]

### 6-(4-(2-Fluoro-5-((4-oxo-7-(2,2,2-trifluoroethoxy)-3,4-dihydrophthalazin-1-yl)methyl)benzoyl)piperazin-1-yl)nicotinonitrile

To a solution of 6-(piperazin-1-yl)nicotinonitrile (12 mg, 0.06 mmol) and 2-fluoro-5-((4-oxo-7-(2,2,2-trifluoroethoxy)-3,4-dihydrophthalazin-1-yl)methyl)benzoic acid (20 mg, 0.05 mmol) in DMF (1 mL) was added EDCI (10 mg, 0.05 mmol), HOBt (10 mg, 0.08 mmol) and DIPEA (20 mg, 0.15 mmol), The reaction mixture was stirred at rt for 2 hours. The reaction mixture was purified by prep-HPLC to afford 6-(4-(2-fluoro-5-((4-oxo-7-(2,2,2-trifluoroethoxy)-3,4-dihydrophthalazin-1-yl)methyl)benzoyl)piperazin-1-yl)nicotinonitrile as a white solid (12 mg, 43% yield). LCMS ESI m/z 567 (M+H)⁺. ¹H NMR (40) MHz, DMSO-d₆) δ 12.53 (s, 1H), 8.51 (d, J=2.1 Hz, 1H), 8.23 (d, J=8.7 Hz, 1H), 7.90 (dd, J=9.1, 2.3 Hz, 1H), 7.56-7.50 (m, 2H), 7.48-7.42 (m, 2H), 7.25 (t, J=8.9 Hz, 1H), 6.92 (d, J=9.2 Hz, 1H), 4.99 (q, J=8.7 Hz, 2H), 4.33 (s, 2H), 3.80-3.70 (m, 4H), 3.65-3.59 (m, 2H), 3.17 (s, 2H).

### Synthesis of Example 10: 6-(4-(2-Fluoro-5-((4-oxo-7-(prop-1-ynyl)-3,4-dihydrophthalazin-1-yl)methyl)benzoyl)piperazin-1-yl)nicotinonitrile

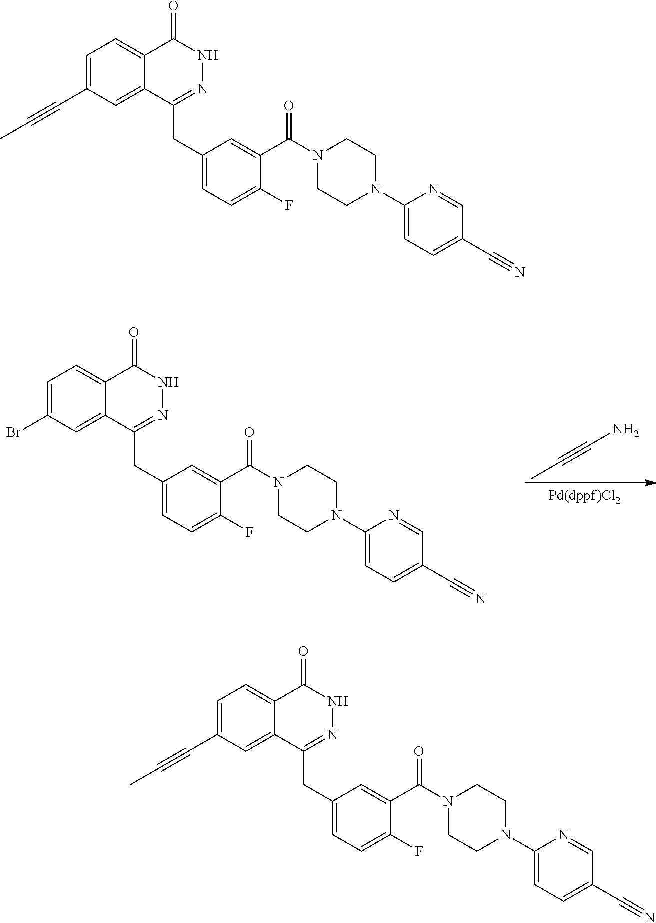

### Step 1

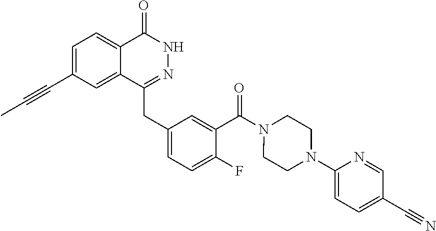 [SMILES: CC#Cc1ccc2c(=O)[nH]nc(Cc3ccc(F)c(C(=O)N4CCN(c5ccc(C#N)cn5)CC4)c3)c2c1]

### 6-(4-(2-Fluoro-5-((4-oxo-7-(prop-1-ynyl)-3,4-dihydrophthalazin-1-yl)methyl)benzoyl)piperazin-1-yl)nicotinonitrile

A mixture of 6-(4-(5-((7-bromo-4-oxo-3,4-dihydrophthalazin-1-yl)methyl)-2-fluorobenzoyl)piperazin-1-yl)nicotinonitrile (420 mg, 0.77 mmol), tributyl(prop-1-ynyl)stannane (760 mg, 2.30 mmol) and Pd(dppf)Cl₂ (58 mg, 0.08 mmol) in 1,4-dioxane (10 mL) were stirred at 100° C. under Ar (g) for 16 hours. The reaction was cooled to rt, diluted with water (20 mL) and extracted with EtOAc (3×20 mL). The combined organic layer was dried over Na₂SO₄, filtered and concentrated in vacuo. The residue was purified by silica gel chromatography (EtOAc/pet ether=50 to 80%) to give 6-(4-(2-fluoro-5-((4-oxo-7-(prop-1-ynyl)-3,4-dihydrophthalazin-1-yl)methyl)benzoyl)piperazin-1-yl)nicotinonitrile tert-butyl as a white solid (145 mg, 37% yield). LCMS ESI m/z 508 (M+H)⁺. ¹H NMR (400 MHz, DMSO-d₆) δ 12.64 (s, 1H), 8.52 (d, J=8.2 Hz, 1H), 8.20 (d, J=8.2 Hz, 1H), 7.98-7.87 (m, 2H), 7.78 (dd, J=8.2, 1.2 Hz, 1H), 7.42-7.26 (m, 3H), 6.93 (d, J=9.1 Hz, 1H), 4.34 (s, 2H), 3.75 (d, J=10.2 Hz, 4H), 3.63 (s, 2H), 3.31 (s, 2H), 2.08 (s, 3H).

### Synthesis of Example 11: 6-(4-(2-Fluoro-5-((7-(3-methoxyazetidin-1-yl)-4-oxo-3,4-dihydrophthalazin-1-yl)methyl)benzoyl)piperazin-1-yl)nicotinonitrile

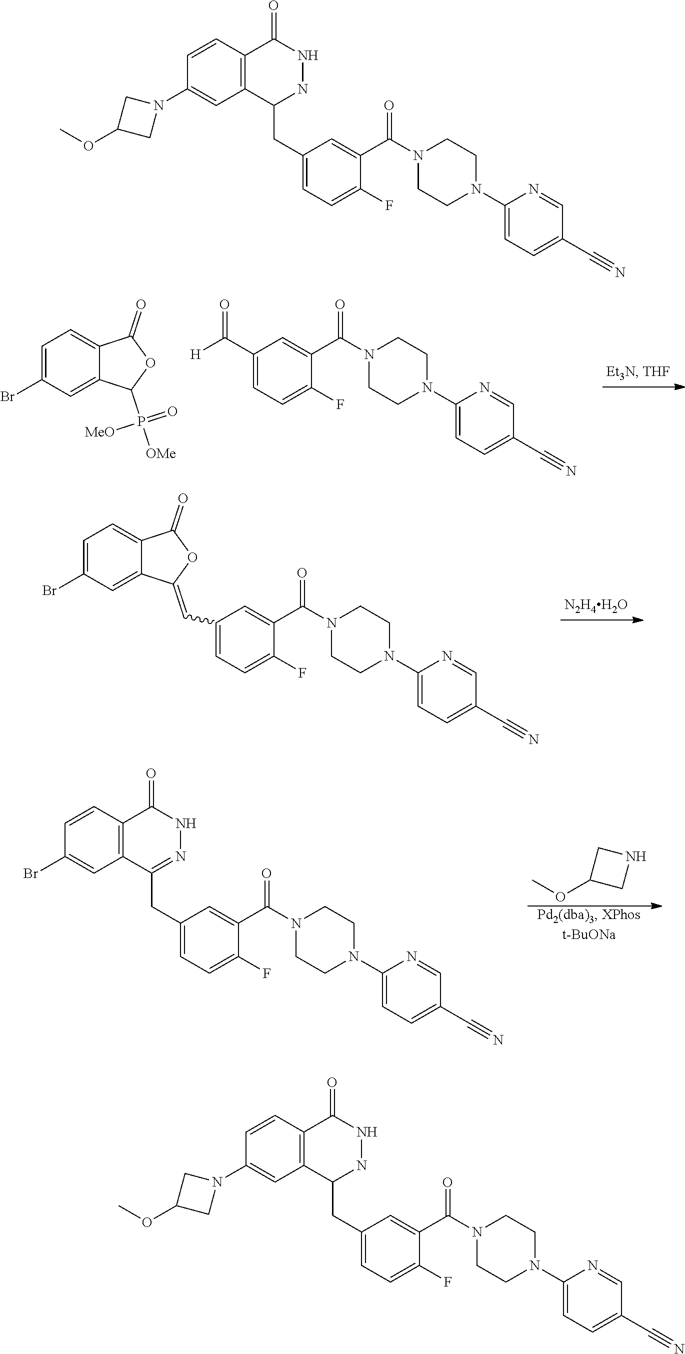

### Step 1

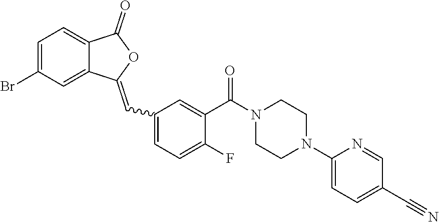 [SMILES: N#Cc1ccc(N2CCN(C(=O)c3cc(C=C4OC(=O)c5ccc(Br)cc54)ccc3F)CC2)nc1]

### 6-(4-(5-((6-Bromo-3-oxoisobenzofuran-1(3H)-ylidene)methyl)-2-fluorobenzoyl)piperazin-1-yl)nicotinonitrile

A mixture of dimethyl (6-bromo-3-oxo-1,3-dihydroisobenzofuran-1-yl)phosphonate (3.5 g, 10.90 mmol), 6-(4-(2-fluoro-5-formylbenzoyl)piperazin-1-yl)nicotinonitrile (4.43 g, 13.08 mmol) and Et₃N (3.31 g, 32.70 mmol) in THF (70 mL) was heated to 60° C. and stirred for 3 hours. The mixture was cooled to rt, diluted with H₂O (50 mL) and extracted with EtOAc (50 mL×3). The combined organic layer was washed with water and brine, dried over anhydrous Na₂SO₄, filtered and concentrated in vacuo. The residue was purified by silica gel chromatography (EtOAc/pet ether=0 to 50%) to afford 6-(4-(5-((6-bromo-3-oxoisobenzofuran-1(3H)-ylidene)methyl)-2-fluorobenzoyl)piperazin-1-yl)nicotinonitrile as a yellow solid (3.6 g, 62% yield). LCMS ESI m/z 533 [M+H]⁺.

### Step 2

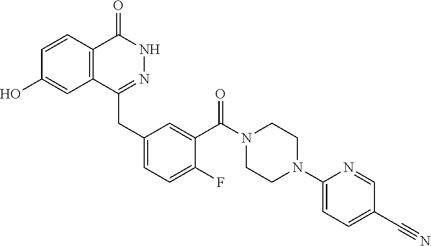 [SMILES: N#Cc1ccc(N2CCN(C(=O)c3cc(Cc4n[nH]c(=O)c5ccc(O)cc45)ccc3F)CC2)nc1]

### 6-(4-(5-((7-Bromo-4-oxo-3,4-dihydrophthalazin-1-yl)methyl)-2-fluorobenzoyl)piperazin-1-yl)nicotinonitrile

To a solution of 6-(4-(5-((6-bromo-3-oxoisobenzofuran-1(3H)-ylidene)methyl)-2-fluorobenzoyl)piperazin-1-yl)nicotinonitrile (3.60 g, 6.75 mmol) in THF (10 mL) was added N₂H₄·H₂O (4.60 g, 67.5 mmol), then the mixture was stirred at 70° C. for 4 hours. The solids were collected by filtration, washed with water and dried under vacuum to give 6-(4-(5-((7-bromo-4-oxo-3,4-dihydrophthalazin-1-yl)methyl)-2-fluorobenzoyl)piperazin-1-yl)nicotinonitrile as a white solid (2.9 g, 79% yield). This was used without further purification. LCMS ESI m/z 547 [M+H]⁺.

### Step 3

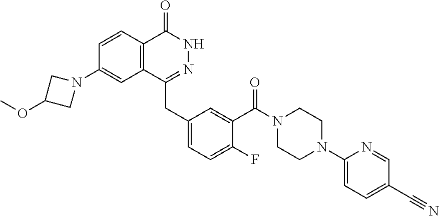 [SMILES: COC1CN(c2ccc3c(=O)[nH]nc(Cc4ccc(F)c(C(=O)N5CCN(c6ccc(C#N)cn6)CC5)c4)c3c2)C1]

### 6-(4-(2-Fluoro-5-((7-(3-methoxyazetidin-1-yl)-4-oxo-3,4-dihydrophthalazin-1-yl)methyl)benzoyl)piperazin-1-yl)nicotinonitrile

A mixture of 6-(4-(5-((7-bromo-4-oxo-3,4-dihydrophthalazin-1-yl)methyl)-2-fluorobenzoyl)piperazin-1-yl)nicotinonitrile (100 mg, 0.18 mmol), Pd₂(dba)₃ (10 mg, 0.01 mmol), XPhos (9.0 mg, 0.02 mmol), sodium tert-butoxide (53 mg, 0.55 mmol) and 3-methoxyazetidine (48 mg, 0.55 mmol) in DMA (2 mL) was stirred at 130° C. for 2 hours. The mixture was cooled to rt, filtered, and the filtrate was purified by prep-HPLC to give 6-(4-(2-fluoro-5-((7-(3-methoxyazetidin-1-yl)-4-oxo-3,4-dihydrophthalazin-1-yl)methyl)benzoyl) piperazin-1-yl)nicotinonitrile as a white solid (11 mg, 18% yield). LCMS ESI m/z 554 (M+H)⁺. ¹H NMR (400 MHz, DMSO-d₆) δ 12.17 (s, 1H), 8.52 (d, J=1.9 Hz, 1H), 8.01 (d, J=8.7 Hz, 1H), 7.91 (dd, J=9.1, 2.3 Hz, 1H), 7.45-7.39 (m, 2H), 7.25 (t, J=9.3 Hz, 1H), 6.92 (d, J=9.1 Hz, 1H), 6.85 (dd, J=8.7, 2.2 Hz, 1H), 6.58 (d, J=2.1 Hz, 1H), 4.39-4.30 (m, 3H), 4.23 (s, 3H), 3.82-3.69 (m, 6H), 3.62 (t, J=7.5 Hz, 2H), 3.24 (s, 4H).

### Synthesis of Example 12: 6-(4-(5-((7-(Cyclopropylamino)-4-oxo-3,4-dihydrophthalazin-1-yl)methyl)-2-fluorobenzoyl)piperazin-1-yl)nicotinonitrile

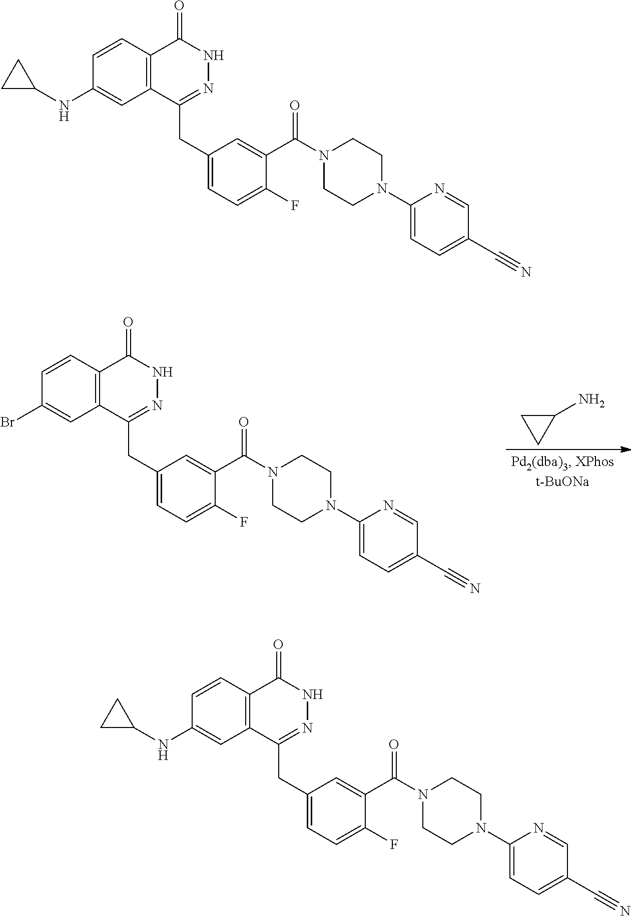

### Step 1

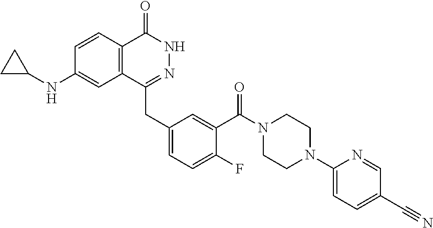 [SMILES: N#Cc1ccc(N2CCN(C(=O)c3cc(Cc4n[nH]c(=O)c5ccc(NC6CC6)cc45)ccc3F)CC2)nc1]

### 6-(4-(5-((7-(Cyclopropylamino)-4-oxo-3,4-dihydrophthalazin-1-yl)methyl)-2-fluorobenzoyl)piperazin-1-yl)nicotinonitrile

A mixture of 6-(4-(5-((7-bromo-4-oxo-3,4-dihydrophthalazin-1-yl)methyl)-2-fluorobenzoyl)piperazin-1-yl)nicotinonitrile (200 mg, 0.36 mmol), 3-methoxyazetidine (63 mg, 1.10 mmol), Pd₂(dba)₃ (20 mg, 0.02 mmol), XPhos (42 mg, 0.09 mmol) and sodium tert-butoxide (70 mg, 0.73 mmol) in DMA (5 mL) was stirred at 130° C. under Ar (g) for 1.5 hours. The mixture was cooled to rt, filtered, and the filtrate was purified by prep-HPLC to give 6-(4-(5-((7-(cyclopropylamino)-4-oxo-3,4-dihydrophthalazin-1-yl)methyl)-2-fluorobenzoyl) piperazin-1-yl)nicotinonitrile as a white solid (14 mg, 7% yield). LCMS ESI m/z 524 (M+H)⁺. ¹H NMR (400 MHz, DMSO-d₆) δ 12.12 (s, 1H), 8.51 (d, J=2.2 Hz, 1H), 7.93 (d, J=8.7 Hz, 1H), 7.90 (dd, J=9.1, 2.3 Hz, 1H), 7.48-7.40 (m, 1H), 7.36 (dd, J=6.4, 2.1 Hz, 1H), 7.26 (t, J=9.0 Hz, 1H), 7.14 (s, 1H), 7.04 (dd, J=8.7, 1.9 Hz, 1H), 6.95-6.88 (m, 2H), 4.21 (s, 2H), 3.82-3.66 (m, 6H), 3.30 (d, J=3.7 Hz, 2H), 2.37 (s, 1H), 0.79-0.70 (m, 2H), 0.37-0.29 (m, 2H).

### Synthesis of Example 13: 6-(4-(5-((7-Cyclopropoxy-4-oxo-3,4-dihydrophthalazin-1-yl)methyl)-2-fluorobenzoyl)piperazin-1-yl)nicotinonitrile

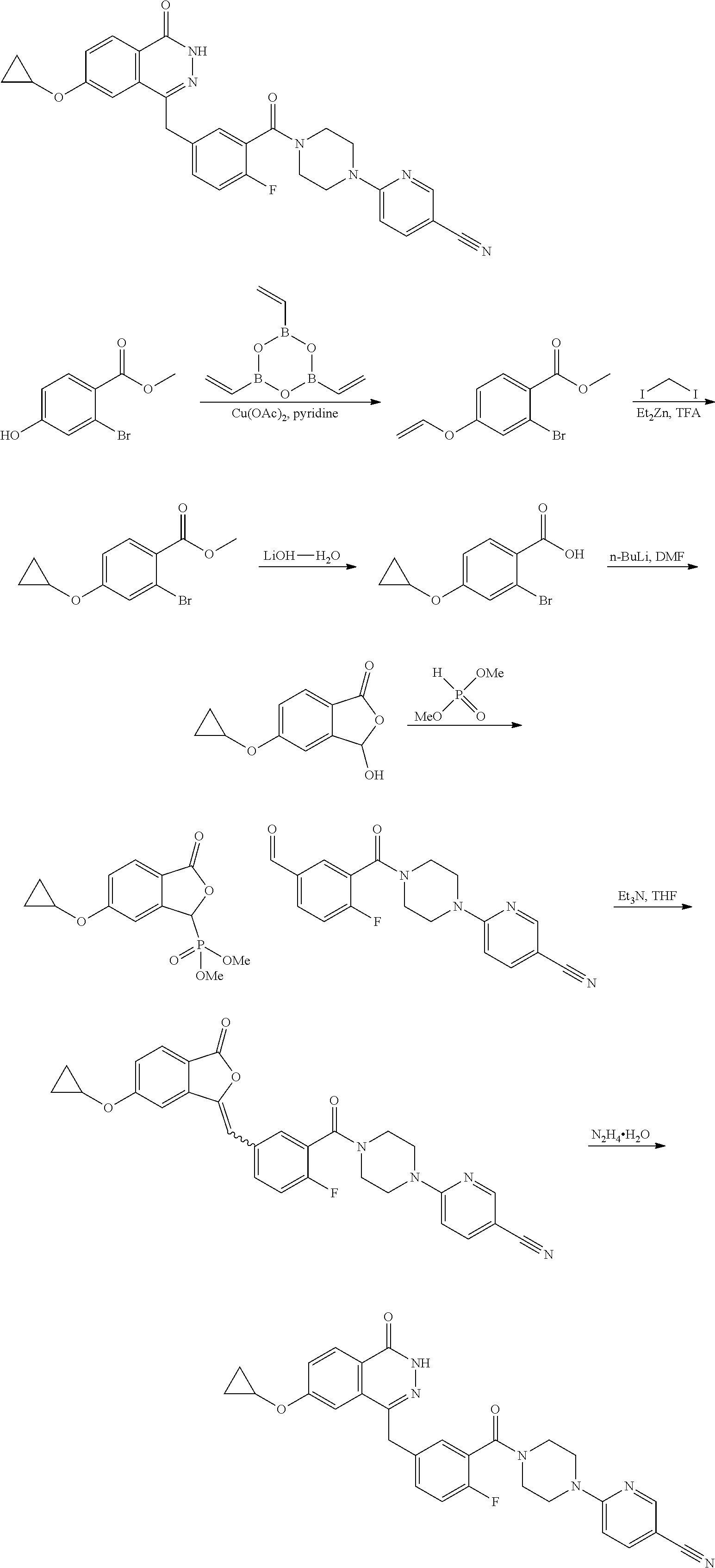

### Step 1

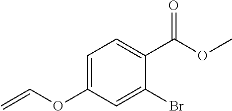

### Methyl 2-bromo-4-(vinyloxy)benzoate

A mixture of methyl 2-bromo-4-hydroxybenzoate (2.00 g, 8.66 mmol), 2,4,6-trivinyl-1,3,5,2,4,6-trioxatriborinane (2.00 g, 8.31 mmol), pyridine (6.85 g, 86.6 mmol) and Cu(OAc)₂ (1.57 g, 8.66 mmol) in DCM (20 mL) was stirred at rt under O₂(g) for 48 hours. The reaction was concentrated in vacuo, then diluted with water (30 mL) and extracted with EtOAc (30 mL×3). The combined organic layer was washed with brine, dried over anhydrous Na₂SO₄, filtered and concentrated in vacuo. The residue was purified by silica gel chromatography (EtOAc/pet ether=0 to 5%) to afford methyl 2-bromo-4-(vinyloxy)benzoate as a yellow oil (1.6 g, 72% yield). LCMS ESI m/z 257 [M+H]⁺.

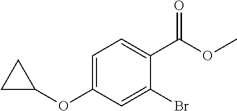

### Methyl 2-bromo-4-cyclopropoxybenzoate

To a solution of Et₂Zn (1 M in hexanes, 35 mL, 35 mmol) in DCM (50 mL) was added TFA (3.99 g, 35 mmol) at 0° C. under N₂ (g). The mixture was stirred for 30 min at 0° C., then diiodomethane (9.38 g, 35 mmol) was added and stirred for another 30 min. To the reaction mixture, a solution of methyl 2-bromo-4-vinyloxy-benzoate (1.5 g, 5.83 mmol) in DCM (10 mL) was added dropwise at 0° C., then stirred at rt for 16 hours. The mixture was quenched with sat.NaHCO₃ (20 mL) and extracted with DCM (50 mL×3). The combined organic layer was dried over anhydrous Na₂SO₄, filtered and concentrated in vacuo. The residue was purified by silica gel chromatography (EtOAc/petroleum ether=0 to 5%) to afford methyl 2-bromo-4-(cyclopropoxy)benzoate (780 mg, 49% yield) as a light yellow oil. LCMS ESI m/z 271 [M+H]⁺. ¹H NMR (400 MHz, CDCl₃) δ 7.83 (d, J=8.8 Hz, 1H), 7.35 (s, 1H), 7.00 (dd, J=8.8, 2.8 Hz, 1H), 3.89 (s, 3H), 3.80-3.75 (m, 1H), 0.84-0.78 (m, 4H).

### Step 3

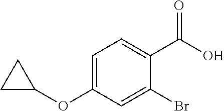 [SMILES: O=C(O)c1ccc(OC2CC2)cc1Br]

### 2-Bromo-4-cyclopropoxybenzoic acid

A mixture of methyl 2-bromo-4-cyclopropoxybenzoate (780 mg, 2.88 mmol) and LiOH—H₂O (605 mg, 14.4 mmol) in MeOH (8 mL) and H₂O (4 mL) was stirred at rt for 3 hours. The organic solvent was removed in vacuo and aq HCl (1N) was added until the solution reached pH 5˜6. The solids were collected by filtration, washed with water and dried under vacuum to give 2-bromo-4-cyclopropoxybenzoic acid as a white solid (640 mg, 87% yield). This was used without further purification. LCMS ESI m/z 257 [M+H]⁺.

### Step 4

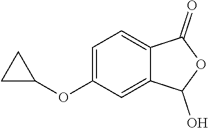 [SMILES: O=C1OC(O)c2cc(OC3CC3)ccc21]

### 5-Cyclopropoxy-3-hydroxyisobenzofuran-1(3H)-one

A mixture of 2-bromo-4-cyclopropoxybenzoic acid (400 mg, 1.57 mmol) in THF (5 mL) was cooled to −78° C., then n-BuLi (2.5 M in heptane, 1.25 mL, 3.11 mmol) was added dropwise under Ar (g). The reaction was stirred at −78° C. for 15 min, then DMF (252 mg, 3.45 mmol) was added. The reaction was kept at −78° C. for 20 min, then quenched with sat. NH₄Cl solution (5 mL). To the resulting mixture, was added aq HCl (1N) until the solution reached pH 5˜6, then extracted with EtOAc (20×3 mL). The combined organic layer was washed with bine, dried over Na₂SO₄, filtered and concentrated in vacuo. The residue was purified by silica gel chromatography (EtOAc/petroleum ether=30 to 50%) to give 5-cyclopropoxy-3-hydroxyisobenzofuran-1(3H)-one as a colorless oil (90 mg, 28% yield). LCMS ESI m/z 207 (M+H)⁺.

### Step 5

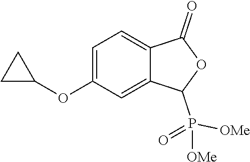 [SMILES: COP(=O)(OC)C1OC(=O)c2ccc(OC3CC3)cc21]

### Dimethyl (6-cyclopropoxy-3-oxo-1,3-dihydroisobenzofuran-1-yl)phosphonate

A mixture of 5-cyclopropoxy-3-hydroxyisobenzofuran-1(3H)-one (90 mg, 0.44 mmol) in dimethyl phosphonate (617 mg, 5.6 mmol) was stirred at 100° C. for 3 hours. The mixture was cooled to rt, diluted with H₂O (10 mL) and extracted with EtOAc (20 mL×3). The combined organic layer was washed with brine, dried over anhydrous Na₂SO₄, filtered and concentrated in vacuo. The residue was purified by silica gel chromatography (EtOAc/petroleum ether=0 to 50%) to give dimethyl (6-cyclopropoxy-3-oxo-1,3-dihydroisobenzofuran-1-yl)phosphonate as a yellow oil (80 mg, 61% yield). LCMS ESI m/z 299 [M+H]⁺.

### Step 6

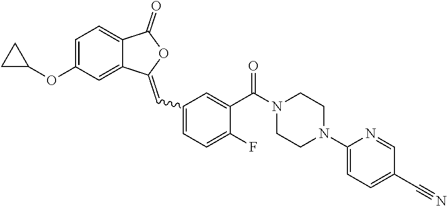 [SMILES: N#Cc1ccc(N2CCN(C(=O)c3cc(C=C4OC(=O)c5ccc(OC6CC6)cc54)ccc3F)CC2)nc1]

### 6-(4-(5-((6-Cyclopropoxy-3-oxoisobenzofuran-1(3H)-ylidene)methyl)-2-fluorobenzoyl)piperazin-1-yl)nicotinonitrile

A mixture of dimethyl(6-cyclopropoxy-3-oxo-1,3-dihydroisobenzofuran-1-yl)phosphonate (80 mg, 0.27 mmol), 6-(4-(2-fluoro-5-formylbenzoyl)piperazin-1-yl)nicotinonitrile (109 mg, 0.32 mmol) and Et₃N (81 mg, 0.80 mmol) in THF (5 mL) was heated to 60° C. and stirred for 3 hours. The mixture was cooled to rt, diluted with H₂O (10 mL) and extracted with DCM (10 mL×3). The combined organic layer was dried over anhydrous Na₂SO₄, filtered and concentrated in vacuo to afford 6-(4-(5-((6-cyclopropoxy-3-oxoisobenzofuran-1(3H)-ylidene)methyl)-2-fluorobenzoyl) piperazin-1-yl)nicotinonitrile as a white solid (130 mg, 95% yield). This was used without further purification. LCMS ESI m/z 511 [M+H]⁺.

### Step 7

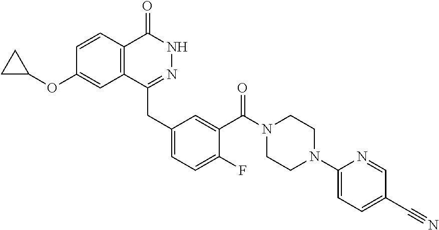 [SMILES: N#Cc1ccc(N2CCN(C(=O)c3cc(Cc4n[nH]c(=O)c5ccc(OC6CC6)cc45)ccc3F)CC2)nc1]

### 6-(4-(5-((7-Cyclopropoxy-4-oxo-3,4-dihydrophthalazin-1-yl)methyl)-2-fluorobenzoyl)piperazin-1-yl)nicotinonitrile

To a solution of 6-(4-(5-((6-cyclopropoxy-3-oxoisobenzofuran-1(3H)-ylidene) methyl)-2-fluorobenzoyl)piperazin-1-yl)nicotinonitrile (140 mg, 0.27 mmol) in THF (5 mL) was added N₂H₄·H₂O (108 mg, 5.4 mmol), then stirred at 60° C. for 3 hours. The reaction was cooled to rt. The solids were collected by filtration, washed with water (5 mL) and dried under vacuo to give 6-(4-(5-((7-cyclopropoxy-4-oxo-3,4-dihydrophthalazin-1-yl)methyl)-2-fluorobenzoyl)piperazin-1-yl)nicotinonitrile as a white solid (60 mg, 42% yield). LCMS ESI m/z 525 [M+H]⁺. ¹H NMR (400 MHz, DMSO-d₆) δ 12.48 (s, 1H), 8.51 (d, J=2.2 Hz, 1H), 8.22-8.12 (m, 1H), 7.90 (dd, J=9.1, 2.3 Hz, 1H), 7.52-7.37 (m, 4H), 7.27 (t, J=9.0 Hz, 1H), 6.93 (d, J=9.1 Hz, 1H), 4.31 (s, 2H), 4.04-3.94 (m, 1H), 3.74 (d, J=12.0 Hz, 4H), 3.61 (d, J=4.0 Hz, 2H), 3.31 (s, 2H), 0.85 (t, J=6.2 Hz, 2H), 0.64 (s, 2H).

### Synthesis of Example 14: 6-(4-(5-((7-Cyclobutoxy-4-oxo-3,4-dihydrophthalazin-1-yl)methyl)-2-fluorobenzoyl)piperazin-1-yl)nicotinonitrile

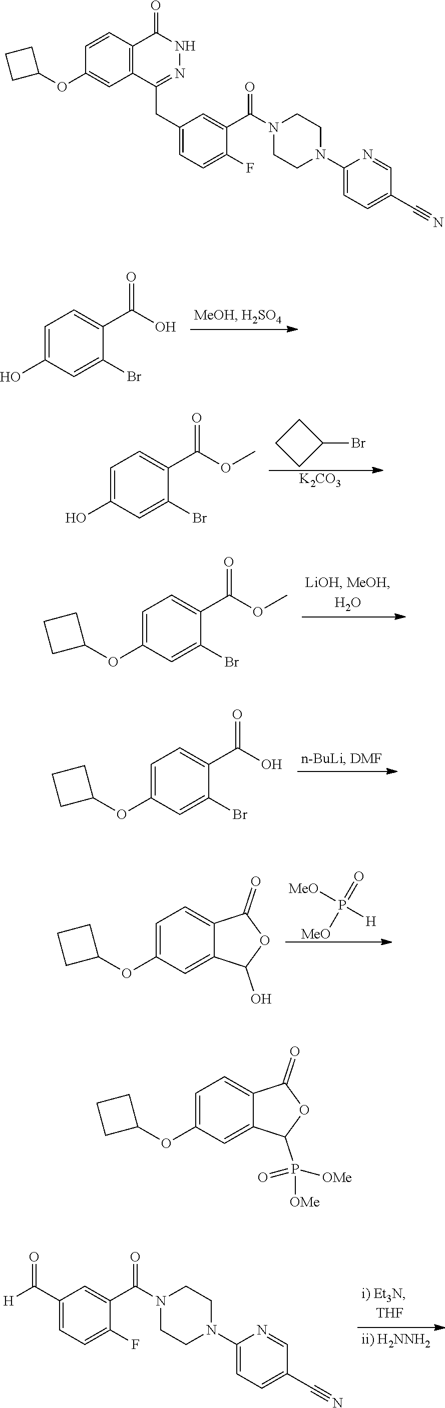

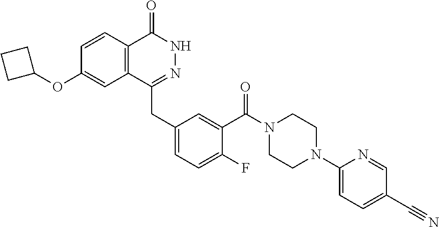 [SMILES: N#Cc1ccc(N2CCN(C(=O)c3cc(Cc4n[nH]c(=O)c5ccc(OC6CCC6)cc45)ccc3F)CC2)nc1]

### Step 1

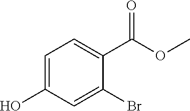

### Methyl 2-bromo-4-hydroxybenzoate

To a solution of 2-bromo-4-hydroxybenzoic (5.00 g, 23.04 mmol) in MeOH (75 mL) was added conc. H₂SO₄ (4.50 g, 23.0 mmol, 2.50 mL) at rt, then stirred at 70° C. for 4 hours. The reaction was concentrated in vacuum, then diluted with water (30 mL) and extracted with EtOAc (20 mL×3). The combined organic layer was washed with brine, dried over anhydrous Na₂SO₄, filtered and concentrated in vacuo to afford methyl 2-bromo-4-hydroxybenzoate as a white solid (5.20 g, 97% yield). This was used without further purification. LCMS ESI m/z 231 [M+H]⁺).

### Step 2

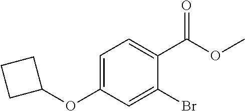 [SMILES: COC(=O)c1ccc(OC2CCC2)cc1Br]

### methyl 2-bromo-4-cyclobutoxybenzoate

A mixture of methyl 2-bromo-4-hydroxybenzoate (1.20 g, 5.19 mmol), K₂CO₃ (2.15 g, 15.6 mmol) and bromocyclobutane (855 mg, 6.34 mmol) in DMF (24 mL) was stirred at 75° C. for 18 hours. The reaction was cooled to rt, diluted with H₂O (5 mL) and extracted with EtOAc (30 mL×3). The combined organic layer was washed with brine, dried over anhydrous Na₂SO₄, filtered and concentrated in vacuum. The residue was purified by silica gel chromatography (EtOAc/petroleum ether=0 to 7%) to give methyl 2-bromo-4-cyclobutoxybenzoate as a colorless oil (1.00 g, 81% yield). LCMS ESI m/z 285 [M+H]⁺).

### Step 3

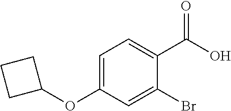 [SMILES: O=C(O)c1ccc(OC2CCC2)cc1Br]

### 2-Bromo-4-cyclobutoxybenzoic acid

A mixture of methyl 2-bromo-4-cyclobutoxybenzoate (1.20 g, 4.21 mmol) and LiOH—H₂O (406 mg, 9.68 mmol) in MeOH (6 mL) and H₂O (2 mL) was stirred at rt for 14 hours. The organic solvent was removed in vacuo and aq HCl (1N) was added until the solution reached pH 5˜6. The solids were collected by filtration, washed with H₂O and dried under vacuum to give 2-bromo-4-cyclobutoxybenzoic acid as a white solid (1.01 g, 88% yield). The product was used without further purification. LCMS ESI m/z 271 [M+H]⁺.

### Step 4

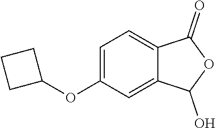 [SMILES: O=C1OC(O)c2cc(OC3CCC3)ccc21]

### 5-Cyclobutoxy-3-hydroxyisobenzofuran-1(3H)-one

A solution of 2-bromo-4-cyclobutoxybenzoic acid (400 mg, 1.48 mmol) in THF (12 mL) was cooled to −78° C., then n-BuLi (2.5 M in heptane, 1.18 mL, 2.96 mmol) was added dropwise under Ar (g). The reaction was stirred at −78° C. for 15 min, then DMF (238 mg, 3.26 mmol) was added. The reaction was stirred at −78° C. for 20 min, then quenched with sat. NH₄Cl solution (10 mL). The mixture was neutralized by aq HCl (1N) until the solution reached pH 5˜6, then extracted with EtOAc (30×3 mL). The combined organic layer was washed with bine, dried over Na₂SO₄, filtered and concentrated in vacuo. The residue was purified by silica gel chromatography (EtOAc/petroleum ether=30 to 50%) to give 5-cyclobutoxy-3-hydroxyisobenzofuran-1(3H)-one as a white solid (80 mg, 24% yield). LCMS ESI m/z 221 (M+H)⁺.

### Step 5

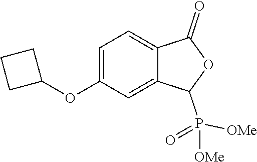 [SMILES: COP(=O)(OC)C1OC(=O)c2ccc(OC3CCC3)cc21]

### Dimethyl (6-cyclobutoxy-3-oxo-1,3-dihydroisobenzofuran-1-yl)phosphonate

A solution of 5-cyclobutoxy-3-hydroxyisobenzofuran-1(3H)-one (80 mg, 1.80 mmol) in dimethylphosphite (6 mL) was stirred at 100° C. for 2 hours. The mixture was cooled to rt, diluted with H₂O (10 mL) and extracted with EtOAc (10 mL×3). The combined organic layer was washed with brine, dried over anhydrous Na₂SO₄, filtered and concentrated in vacuo. The residue was purified by silica gel chromatography (EtOAc/petroleum ether=50 to 60%) to give dimethyl (6-cyclobutoxy-3-oxo-1,3-dihydroisobenzofuran-1-yl)phosphonate as a colorless oil (62 mg, 54% yield). LCMS ESI m/z 313 [M+H]⁺.

### Step 6

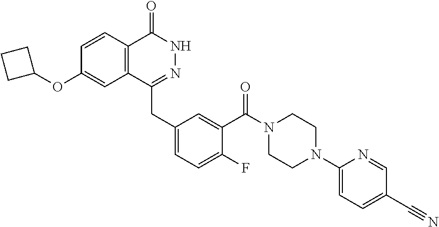 [SMILES: N#Cc1ccc(N2CCN(C(=O)c3cc(Cc4n[nH]c(=O)c5ccc(OC6CCC6)cc45)ccc3F)CC2)nc1]

### 6-(4-(2,3-Difluoro-5-((4-oxo-7-(prop-1-ynyl)-3,4-dihydrophthalazin-1-yl)methyl)benzoyl)piperazin-1-yl)nicotinonitrile

A solution of dimethyl (6-cyclobutoxy-3-oxo-1,3-dihydroisobenzofuran-1-yl)phosphonate (50 mg, 0.16 mmol), 6-(4-(2-fluoro-5-formylbenzoyl)piperazin-1-yl)nicotinonitrile (65 mg, 0.19 mmol) and Et₃N (49 mg, 0.48 mmol) in anhydrous THF (5 mL) was stirred at 25° C. under Ar (g) for 15 hours. Hydrazine hydrate (50 mg, 1 mmol) was added and the reaction was stirred at 70° C. for 1 hour. The reaction was cooled to rt, then THF was removed under vacuum. The formed solid was washed with water (10 mL) and petroleum ether/EtOAc (2:1, 10 mL) to give methyl 5-[(7-bromo-4-oxo-3H-phthalazin-1-yl)methyl]-2,3-difluoro-benzoate as a white solid (35 mg, 41% yield). LCMS ESI m/z 539 [M+H]). ¹H NMR (400 MHz, CDCl₃) δ 10.04 (s, 1H), 8.42 (d, J=2.2 Hz, 1H), 8.35 (d, J=8.8 Hz, 1H), 7.65 (dd, J=9.0, 2.3 Hz, 1H), 7.38-7.30 (m, 2H), 7.20 (dd, J=8.8, 2.4 Hz, 1H), 7.07 (t, J=8.8 Hz, 1H), 6.91 (d, J=2.3 Hz, 1H), 6.62 (d, J=9.0 Hz, 1H), 4.67 (d, J=7.0 Hz, 1H), 4.22 (s, 2H), 3.91-3.87 (m, 2H), 3.79-3.76 (m, 2H), 3.69 (m, 2H), 3.54-3.33 (m, 2H), 2.42 (m, 2H), 2.23-2.09 (m, 2H), 1.96-1.84 (m, 1H), 1.84-1.70 (m, 1H).

### Synthesis of Example 15: 6-(4-(2-Fluoro-5-((4-oxo-7-(prop-2-ynylamino)-3,4-dihydrophthalazin-1-yl)methyl)benzoyl)piperazin-1-yl)nicotinonitrile

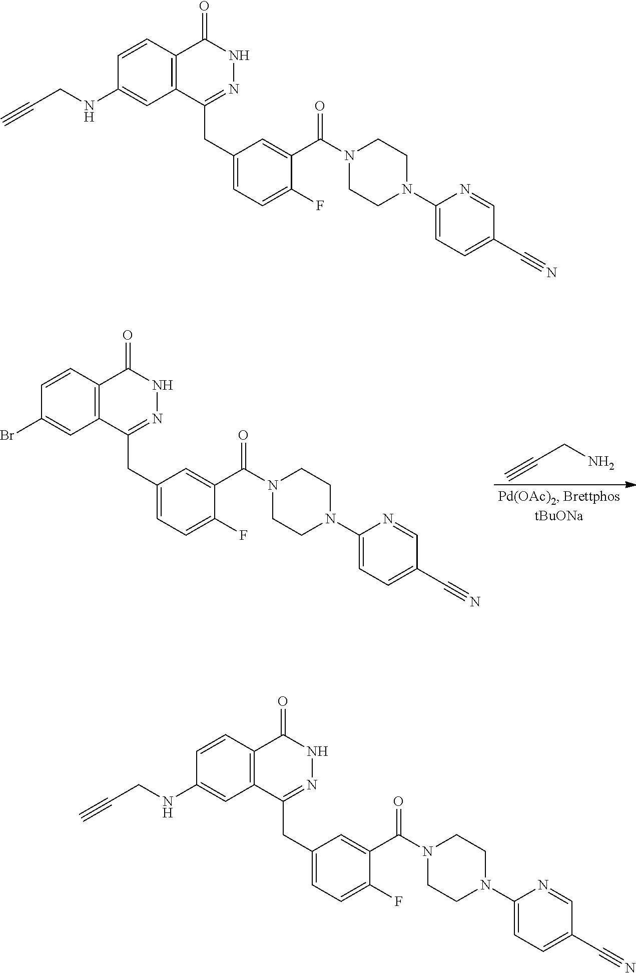

### Step 1

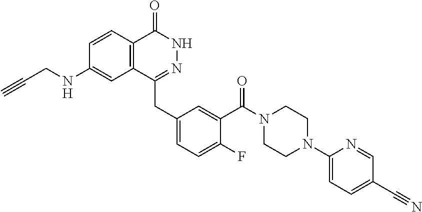 [SMILES: C#CCNc1ccc2c(=O)[nH]nc(Cc3ccc(F)c(C(=O)N4CCN(c5ccc(C#N)cn5)CC4)c3)c2c1]

### 6-(4-(2-Fluoro-5-((4-oxo-7-(prop-2-ynylamino)-3,4-dihydrophthalazin-1-yl)methyl)benzoyl)piperazin-1-yl)nicotinonitrile

A mixture of 6-(4-(5-((7-bromo-4-oxo-3,4-dihydrophthalazin-1-yl)methyl)-2-fluorobenzoyl)piperazin-1-yl)nicotinonitrile (200 mg, 0.37 mmol), prop-2-yn-1-amine (403 mg, 7.33 mmol), Pd(OAc)₂ (17 mg, 0.07 mmol), Brettphos (37 mg, 0.07 mmol) and sodium tert-butoxide (71 mg, 0.74 mmol) in 1,4-dioxane (10 mL) was stirred at 100° C. for 8 hours. The reaction was cooled to rt, then the solids were filtered off. The filtrate was concentrated in vacuo and the residue was purified by prep-HPLC to give 6-(4-(2-fluoro-5-((4-oxo-7-(prop-2-ynylamino)-3,4-dihydrophthalazin-1-yl)methyl)benzoyl)piperazin-1-yl)nicotinonitrile as a white solid (35 mg, 18% yield). LCMS ESI m/z 522 (M+H)⁺. ¹H NMR (400 MHz, DMSO-d₆) δ 12.66 (s, 1H), 8.51 (d, J=2.2 Hz, 1H), 8.23 (d, J=8.2 Hz, 1H), 7.93-7.89 (m, 2H), 7.79 (dd, J=8.2, 1.3 Hz, 1H), 7.44-7.37 (m, 2H), 7.26 (t, J=9.0 Hz, 1H), 6.93 (d, J=9.1 Hz, 1H), 4.35 (s, 2H), 3.79-3.70 (m, 4H), 3.65-3.57 (m, 4H), 3.31-3.27 (m, 2H), 2.76 (s, 1H).

### Synthesis of Example 16: 6-(4-(2-Fluoro-5-((7-(methylamino)-4-oxo-3,4-dihydrophthalazin-1-yl)methyl)benzoyl) piperazin-1-yl)nicotinonitrile

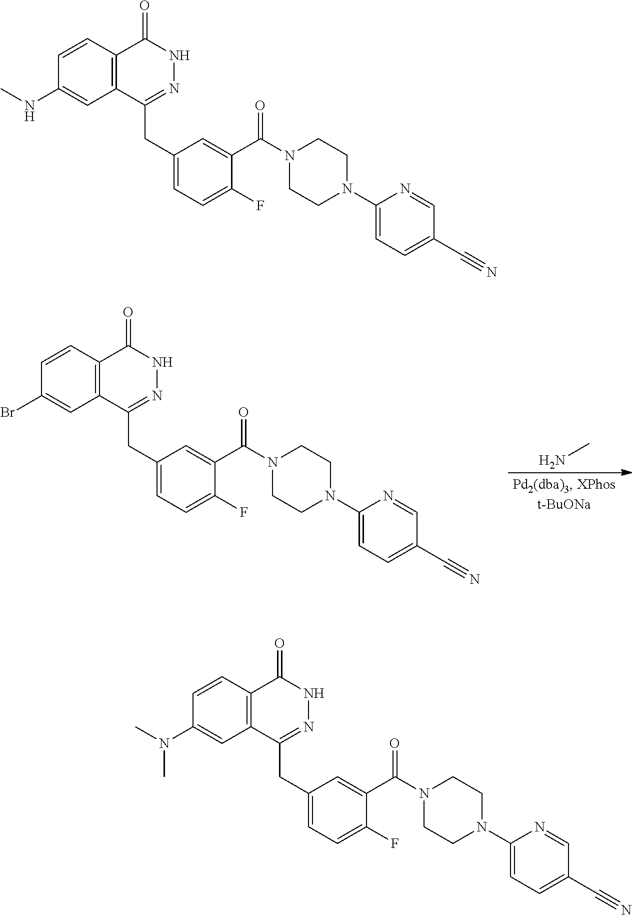

### Step 1

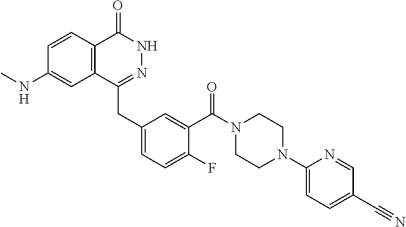 [SMILES: CNc1ccc2c(=O)[nH]nc(Cc3ccc(F)c(C(=O)N4CCN(c5ccc(C#N)cn5)CC4)c3)c2c1]

### 6-(4-(2-Fluoro-5-((7-(methylamino)-4-oxo-3,4-dihydrophthalazin-1-yl)methyl)benzoyl) piperazin-1-yl)nicotinonitrile

To a solution of 6-(4-(5-((7-bromo-4-oxo-3,4-dihydrophthalazin-1-yl)methyl)-2-fluorobenzoyl)piperazin-1-yl)nicotinonitrile (50.0 mg, 91.3 μmol) in DMA (3 mL) was added methanamine hydrochloride (47.49 mg, 456.72 μmol, HCl), t-BuONa (61.45 mg, 0.639 mmol), Pd₂(dba)₃ (8.4 mg, 9.1 μmol), and XPhos (8.71 mg, 18.3 μmol). This reaction mixture was heated to 130° C. for 16 hours. The mixture was purified by Prep-HPLC to give 6-(4-(2-fluoro-5-((7-(methylamino)-4-oxo-3,4-dihydrophthalazin-1-yl)methyl)benzoyl)piperazin-1-yl)nicotinonitrile (10.0 mg, 20% yield) as a white solid. LCMS ESI m/z 497.7 [M+H]⁺. ¹H NMR (400 MHz, DMSO-d₆) δ 12.07 (s, 1H), 8.51 (d, J=2.1 Hz, 1H), 7.95-7.86 (m, 2H), 7.47-7.35 (m, 2H), 7.25 (t, J=9.0 Hz, 1H), 6.98 (dd, J=8.8, 2.1 Hz, 1H), 6.92 (d, J=9.1 Hz, 1H), 6.78 (d, J=5.0 Hz, 1H), 6.63 (d, J=2.0 Hz, 1H), 4.21 (s, 2H), 3.81-3.68 (m, 4H), 3.60 (s, 2H), 3.31-3.28 (m, 2H), 2.73 (d, J=4.9 Hz, 3H).

### Synthesis of Example 17: 6-(4-(2-Fluoro-5-((7-(methyl(prop-2-ynyl)amino)-4-oxo-3,4-dihydrophthalazin-1-yl)methyl)benzoyl)piperazin-1-yl)nicotinonitrile

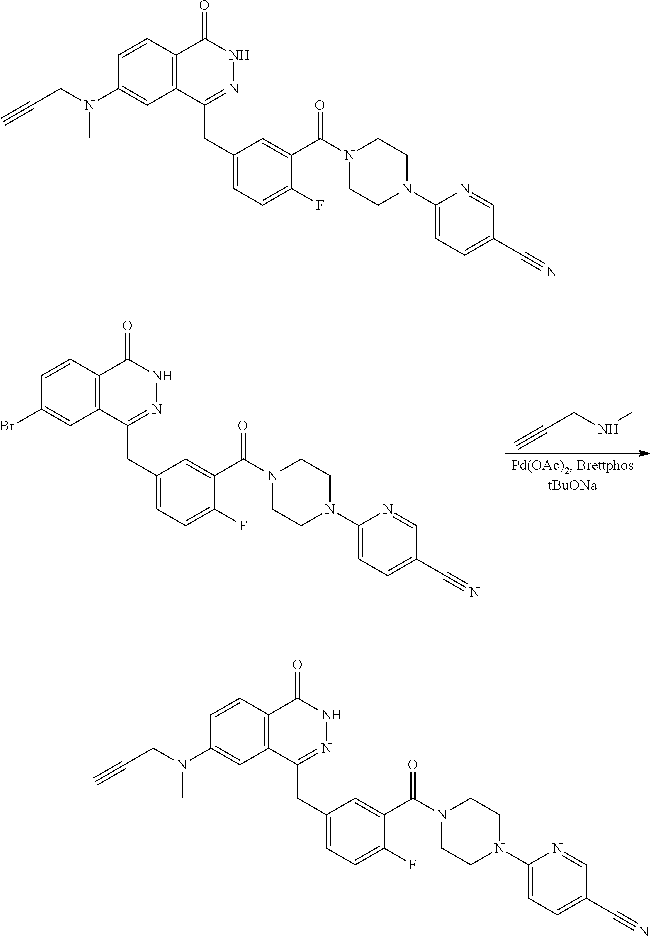

### Step 1

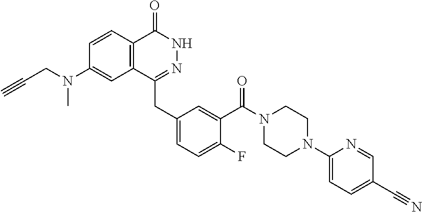 [SMILES: C#CCN(C)c1ccc2c(=O)[nH]nc(Cc3ccc(F)c(C(=O)N4CCN(c5ccc(C#N)cn5)CC4)c3)c2c1]

### 6-(4-(2-Fluoro-5-((7-(methyl(prop-2-ynyl)amino)-4-oxo-3,4-dihydrophthalazin-1-yl)methyl)benzoyl)piperazin-1-yl)nicotinonitrile

A mixture of 6-(4-(5-((7-bromo-4-oxo-3,4-dihydrophthalazin-1-yl)methyl)-2-fluorobenzoyl)piperazin-1-yl)nicotinonitrile (200 mg, 0.37 mmol), N-methylprop-2-yn-1-amine (505 mg, 7.33 mmol), Pd(OAc)₂ (17 mg, 0.07 mmol), Brettphos (37 mg, 0.07 mmol) and sodium tert-butoxide (71 mg, 0.74 mmol) in 1,4-dioxane (10 mL) was stirred at 100° C. for 8 hours. The reaction was cooled to rt, then the solids were filtered off. The filtrate was concentrated in vacuo and the residue was purified by silica gel chromatography (EtOAc/petroleum ether=80% to 100/0) to give 6-(4-(2-fluoro-5-((7-(methyl(prop-2-ynyl)amino)-4-oxo-3,4-dihydrophthalazin-1-yl)methyl) benzoyl) piperazin-1-yl)nicotinonitrile as a white solid (45 mg, 23% Yield). LCMS ESI m/z 536 (M+H)⁺. ¹H NMR (400 MHz, MeOD) δ 8.42 (d, J=1.9 Hz, 1H), 8.32 (d, J=8.2 Hz, 1H), 7.95 (s, 1H), 7.76 (dd, J=9.1, 2.3 Hz, 2H), 7.50-7.45 (m, 1H), 7.40-7.36 (m, 1H), 7.18 (t, J=9.0 Hz, 1H), 6.87 (d, J=9.1 Hz, 1H), 4.37 (s, 2H), 3.86-3.81 (m, 4H), 3.70-3.66 (m, 4H), 3.44-3.39 (m, 2H), 2.51 (s, 3H).

### Synthesis of Example 18: 6-(4-(5-((7-(3,3-Difluoroazetidin-1-yl)-4-oxo-3,4-dihydrophthalazin-1-yl)methyl)-2-fluorobenzoyl)piperazin-1-yl)nicotinonitrile

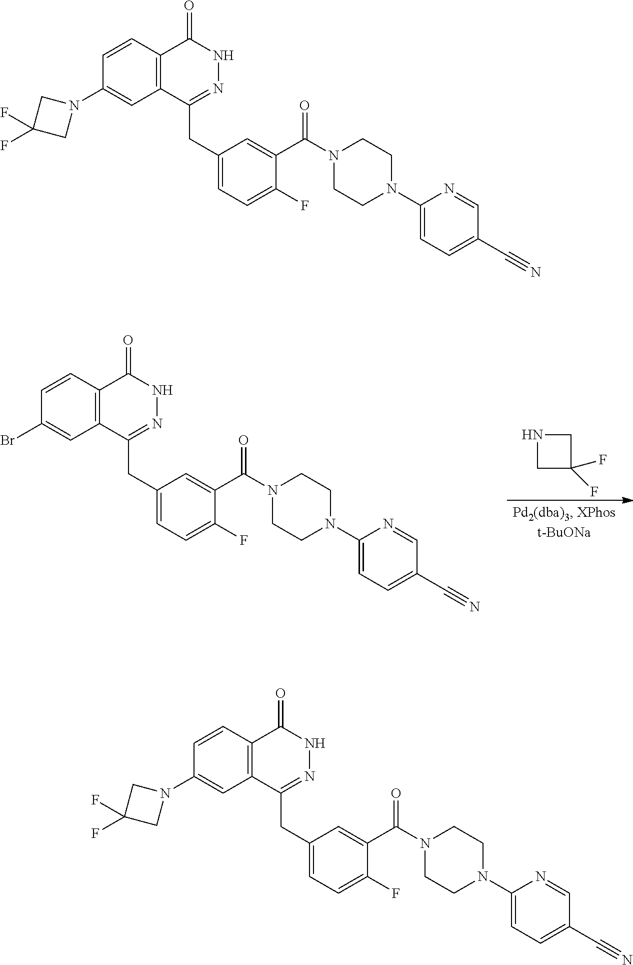 [SMILES: N#Cc1ccc(N2CCN(C(=O)c3cc(Cc4n[nH]c(=O)c5ccc(N6CC(F)(F)C6)cc45)ccc3F)CC2)nc1 ; N#Cc1ccc(N2CCN(C(=O)c3cc(Cc4n[nH]c(=O)c5ccc(Br)cc45)ccc3F)CC2)nc1 ; FC1(F)CNC1 ; N#Cc1ccc(N2CCN(C(=O)c3cc(Cc4n[nH]c(=O)c5ccc(N6CC(F)(F)C6)cc45)ccc3F)CC2)nc1]

### Step 1

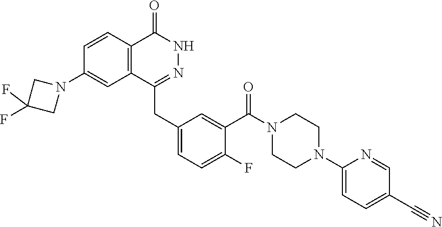 [SMILES: N#Cc1ccc(N2CCN(C(=O)c3cc(Cc4n[nH]c(=O)c5ccc(N6CC(F)(F)C6)cc45)ccc3F)CC2)nc1]

### 6-(4-(5-((7-(3,3-Difluoroazetidin-1-yl)-4-oxo-3,4-dihydrophthalazin-1-yl)methyl)-2-fluorobenzoyl)piperazin-1-yl)nicotinonitrile

To a solution of 6-[4-[5-[(7-bromo-4-oxo-3H-phthalazin-1-yl)methyl]-2-fluoro-benzoyl]piperazin-1-yl]pyridine-3-carbonitrile (99.6 mg, 182 μmol) in DMA (2 mL) was added 3,3-difluoroazetidine hydrochloride (118 mg, 910 μmol), t-BuONa (122 mg, 1.27 mmol), Pd₂(dba)₃ (16.7 mg, 18.2 μmol), and XPhos (17.4 mg, 36.4 μmol). The resulting mixture was heated to 130° C. for 16 hours. The mixture was purified by Prep-HPLC to give 6-[4-[5-[[7-(3,3-difluoroazetidin-1-yl)-4-oxo-3H-phthalazin-1-yl]methyl]-2-fluoro-benzoyl]piperazin-1-yl]pyridine-3-carbonitrile (08.03 mg, 16% yield) as a white solid. LCMS ESI m/z 559.7 [M+H]⁺. ¹H NMR (400 MHz, DMSO-d₆) δ 12.27 (s, 1H), 8.52 (d, J=2.0 Hz, 1H), 8.09 (d, J=8.7 Hz, 1H), 7.90 (dd, J=9.1, 2.4 Hz, 11f), 7.48-7.42 (m, 2H), 7.26 (t, J=9.3 Hz, 1H), 7.01 (dd, J=8.7, 2.2 Hz, 1H), 6.93 (d, J=9.1 Hz, 1H), 6.82 (d, J=2.2 Hz, 1H), 4.44 (t, J=12.3 Hz, 4H), 4.26 (s, 2H), 3.76 (m, 4H), 3.63 (m, 2H), 3.36 (m, 2H).

### Synthesis of Example 19: 6-(4-(5-((7-(But-2-ynyloxy)-4-oxo-3,4-dihydrophthalazin-1-yl)methyl)-2-fluorobenzoyl)piperazin-1-yl)nicotinonitrile

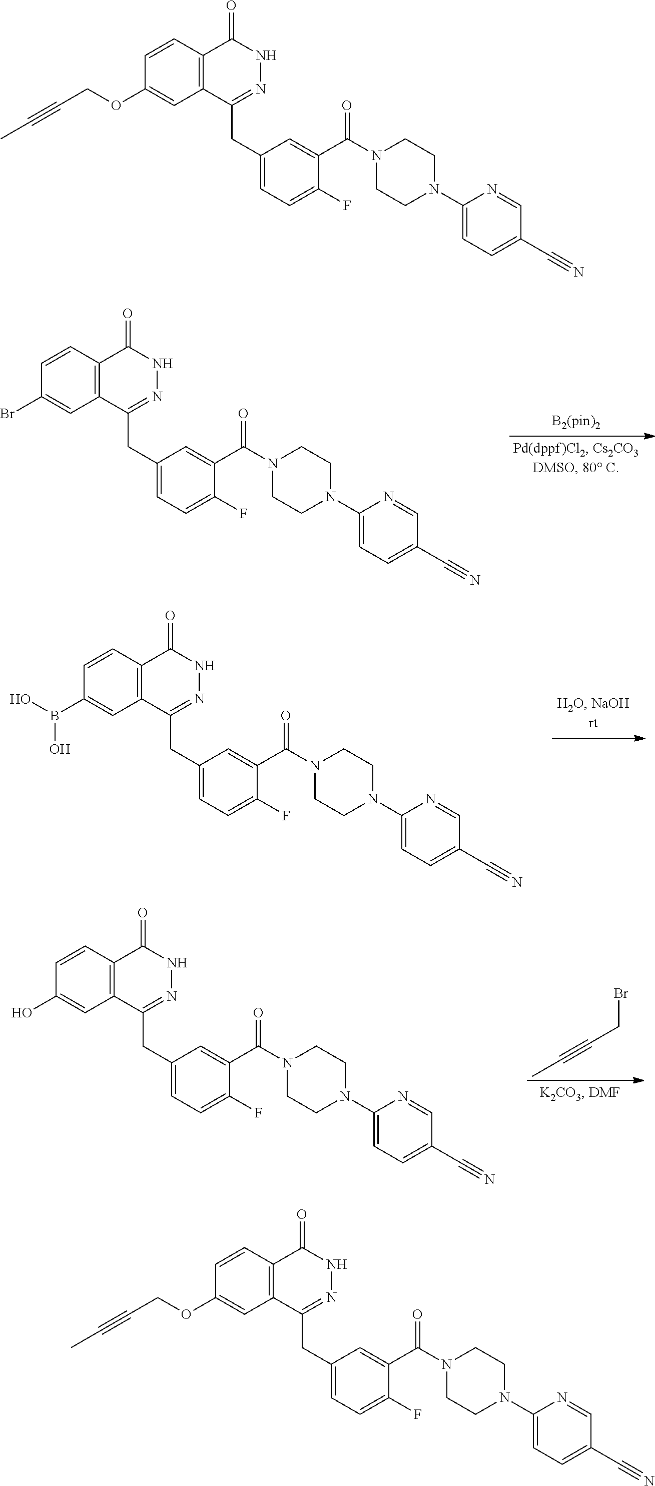

### Step 1

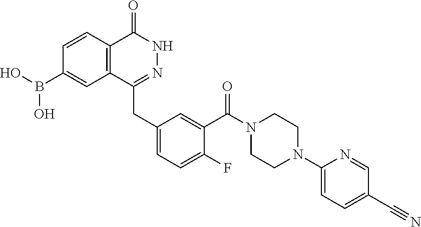 [SMILES: N#Cc1ccc(N2CCN(C(=O)c3cc(Cc4n[nH]c(=O)c5ccc(B(O)O)cc45)ccc3F)CC2)nc1]

### 4-(3-(4-(5-cyanopyridin-2-yl)piperazine-1-carbonyl)-4-fluorobenzyl)-1-oxo-1,2-dihydrophthalazin-6-ylboronic acid

A mixture of 6-(4-(5-((7-bromo-4-oxo-3,4-dihydrophthalazin-1-yl)methyl)-2-fluorobenzoyl)piperazin-1-yl)nicotinonitrile (400 mg, 0.73 mmol), 4,4,4′,4′,5,5,5′,5′-octamethyl-2,2′-bi(1,3,2-dioxaborolane) (556 mg, 2.19 mmol), Cs₂CO₃ (714 mg, 2.19 mmol) and Pd(dppf)Cl₂ (58 mg, 0.08 mmol) in DMSO (10 mL) was stirred at 80° C. under Ar (g) for 16 hours. The reaction was cooled to rt, diluted with water (20 mL) and extracted with EtOAc (3×20 mL). The combined organic layer was dried over Na₂SO₄, filtered and concentrated in vacuo. The residue was purified by silica gel chromatography (DCM/MeOH=0% to 10%) to give 4-(3-(4-(5-cyanopyridin-2-yl)piperazine-1-carbonyl)-4-fluorobenzyl)-1-oxo-1,2-dihydrophthalazin-6-ylboronic acid (240 mg, 64% yield) as a white solid. LCMS ESI m/z: 513 [M+H]⁺.

### Step 2

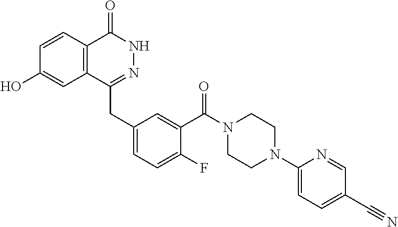 [SMILES: N#Cc1ccc(N2CCN(C(=O)c3cc(Cc4n[nH]c(=O)c5ccc(O)cc45)ccc3F)CC2)nc1]

### 6-(4-(2-fluoro-5-((7-hydroxy-4-oxo-3,4-dihydrophthalazin-1-yl)methyl)benzoyl) piperazin-1-yl)nicotinonitrile

A solution of 4-(3-(4-(5-cyanopyridin-2-yl)piperazine-1-carbonyl)-4-fluorobenzyl)-1-oxo-1,2-dihydrophthalazin-6-ylboronic acid (240 mg, 0.47 mmol) and H₂O₂ (180 mg, 4.70 mmol) in dioxane (8 mL) were stirred at rt for 10 mins, then NaOH (56 mg, 1.41 mmol) in H₂O (1 mL) was added. The reaction mixture was stirred rt for 16 hours, diluted with H₂O (10 mL) and extracted with EtOAc (3×20 mL). The combined organic lay er was dried over Na₂SO₄, filtered and concentrated in vacuo. The residue was purified by silica gel chromatography (DCM/MeOH=0% to 10%/o) to give 6-(4-(2-fluoro-5-((7-hydroxy-4-oxo-3,4-dihydrophthalazin-1-yl)methyl)benzoyl)piperazin-1-yl)nicotinonitrile (40 mg, 18% yield) as a white solid. LCMS ESI m/z: 485 [M+H]⁺.

### Step 3

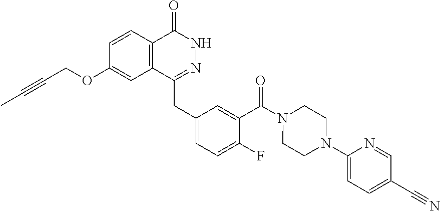 [SMILES: CC#CCOc1ccc2c(=O)[nH]nc(Cc3ccc(F)c(C(=O)N4CCN(c5ccc(C#N)cn5)CC4)c3)c2c1]

### 6-(4-(5-((7-(but-2-ynyloxy)-4-oxo-3,4-dihydrophthalazin-1-yl)methyl)-2-fluon)benzoyl)piperazin-1-yl)nicotinonitrile

A mixture of 6-(4-(2-fluoro-5-((7-hydroxy-4-oxo-3,4-dihydrophthalazin-1-yl)methyl)benzoyl)piperazin-1-yl)nicotinonitrile (40 mg, 0.08 mmol), 1-bromobut-2-yne (16 mg, 0.12 mmol) and K₂CO₃ (33 mg, 0.24 mmol) in DMF (5 mL) was stirred at 50° C. for 1 hour. The solids were filtered off, and the filtrate was concentrated in vacuo. The residue was purified by pre-HPLC to give 2-chloro-4-isopropoxy-1,8-naphthyridine (25 mg, 58% yield) as a white solid. LCMS ESI m/z: 537 [M+H]⁺. ¹H NMR (400 MHz, DMSO-D6) δ 12.48 (s, 1H), 8.52 (d, J=2.2 Hz, 1H), 8.19 (d, J=8.8 Hz, 1H), 7.90 (dd, J=9.1, 2.3 Hz, 1H), 7.48-7.39 (m, 3H), 7.36-7.33 (m, 1H), 7.26 (t, J=9.0 Hz, 1H), 6.93 (d, J=9.1 Hz, 1H), 4.91 (d, J=2.2 Hz, 2H), 4.30 (s, 2H), 3.75 (m, 4H), 3.62 (m, 2H), 3.32-3.30 (m, 2H), 1.78 (s, 3H).

### Synthesis of Example 20: 6-(4-(5-((7-(cyclopropylmethylamino)-4-oxo-3,4-dihydrophthalazin-1-yl)methyl)-2-fluorobenzoyl)piperazin-1-yl)nicotinonitrile

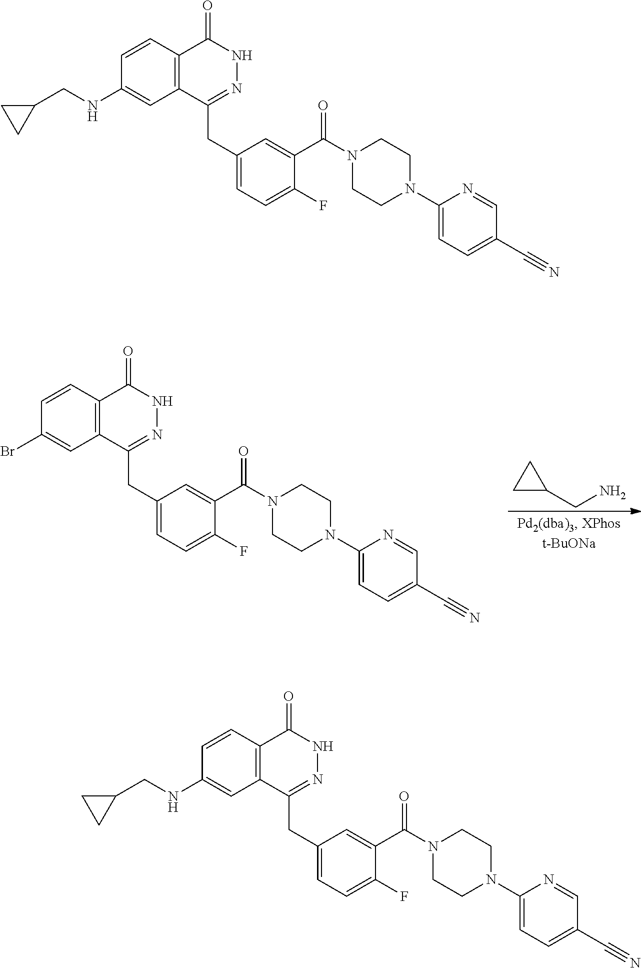 [SMILES: N#Cc1ccc(N2CCN(C(=O)c3cc(Cc4n[nH]c(=O)c5ccc(NCC6CC6)cc45)ccc3F)CC2)nc1 ; N#Cc1ccc(N2CCN(C(=O)c3cc(Cc4n[nH]c(=O)c5ccc(Br)cc45)ccc3F)CC2)nc1 ; NCC1CC1 ; N#Cc1ccc(N2CCN(C(=O)c3cc(Cc4n[nH]c(=O)c5ccc(NCC6CC6)cc45)ccc3F)CC2)nc1]

### Step 1

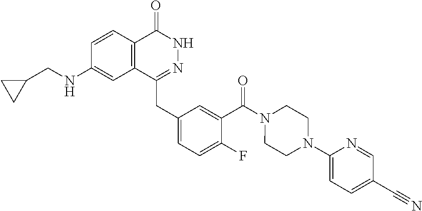 [SMILES: N#Cc1ccc(N2CCN(C(=O)c3cc(Cc4n[nH]c(=O)c5ccc(NCC6CC6)cc45)ccc3F)CC2)nc1]

### 6-(4-(5-((7-(Cyclopropylmethylamino)-4-oxo-3,4-dihydrophthalazin-1-yl)methyl)-2-fluorobenzoyl)piperazin-1-yl)nicotinonitrile

A mixture of 6-(4-(5-((7-bromo-4-oxo-3,4-dihydrophthalazin-1-yl)methyl)-2-fluorobenzoyl)piperazin-1-yl)nicotinonitrile (200 mg, 0.37 mmol), cyclopropylmethanamine (105 mg, 1.48 mmol), Pd₂(dba)₃ (65 mg, 0.07 mmol), XPhos (33 mg, 0.07 mmol) and t-BuONa (107 mg, 1.11 mmol) in DMA (10 mL) was stirred at 130° C. for 14 hours. The reaction was cooled to rt, then the solids were filtered off. The filtrate was concentrated in vacuo and the residue was purified by prep-HPLC to give 6-(4-(5-((7-(cyclopropylmethylamino)-4-oxo-3,4-dihydrophthalazin-1-yl)methyl)-2-fluorobenzoyl)piperazin-1-yl)nicotinonitrile as a white solid (30 mg, 15% Yield). LCMS ESI m/z 538 (M+H)⁺. ¹H NMR (400 MHz, DMSO-d₆) δ 12.06 (s, 1H), 8.51 (d, J=2.1 Hz, 1H), 7.90 (dd, J=9.0, 2.5 Hz, 2H), 7.46-7.40 (m, 1H), 7.39-7.34 (m, 1H), 7.25 (t, J=9.0 Hz, 1H), 7.03 (dd, J=8.9, 2.1 Hz, 1H), 6.92 (d, J=9.2 Hz, 1H), 6.86 (t, J=5.2 Hz, 1H), 6.69 (s, 1H), 4.20 (s, 2H), 3.76 (s, 2H), 3.73 (s, 2H), 3.60 (s, 2H), 3.31-3.27 (m, 2H), 2.95 (t, 2H), 1.05-0.90 (m, 1H), 0.48-0.40 (m, 2H), 0.24-0.15 (m, 2H).

### Synthesis of Example 21: 6-(4-(5-((7-(Difluoromethoxy)-4-oxo-3,4-dihydrophthalazin-1-yl)methyl)-2-fluorobenzoyl)piperazin-1-yl)nicotinonitrile

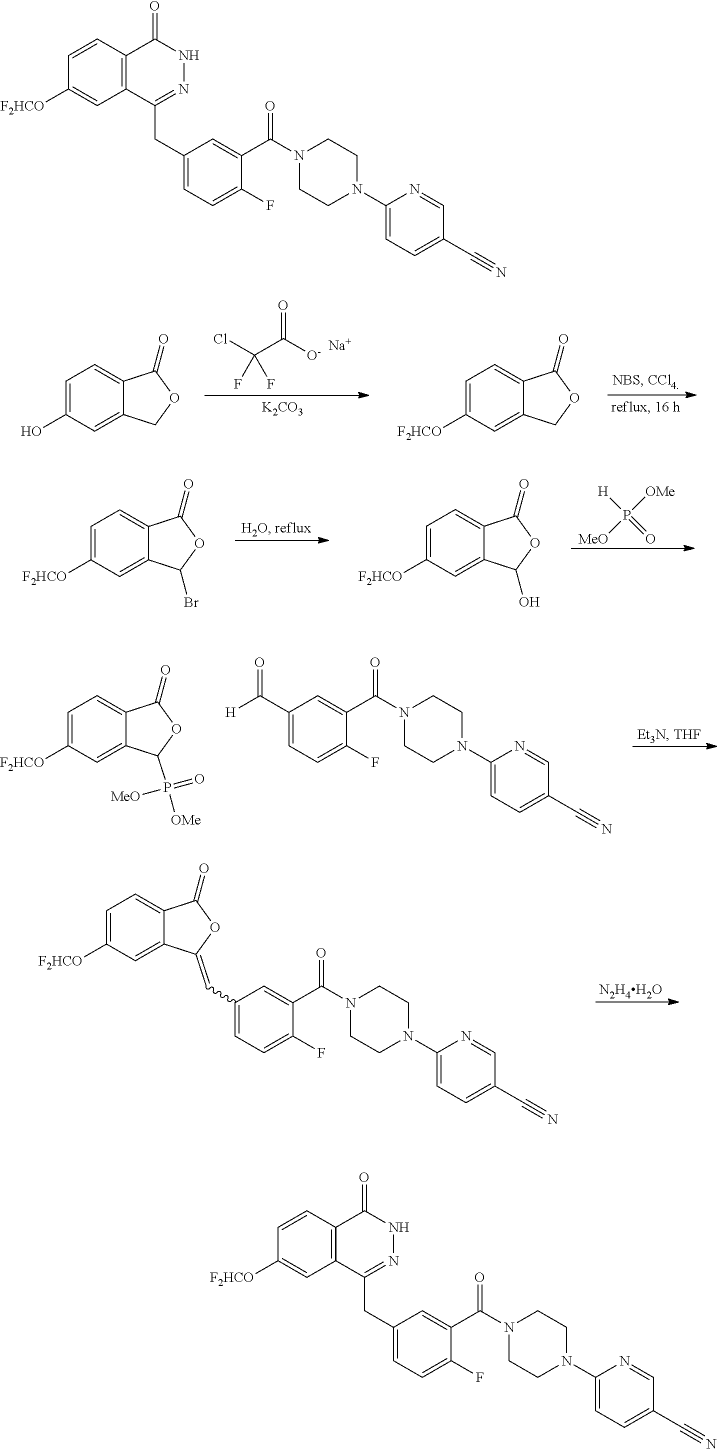

### Step 1

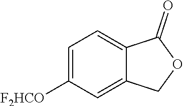 [SMILES: O=C1OCc2cc(OC(F)F)ccc21]

### 5-(Difluoromethoxy)isobenzofuran-1(3H)-one

To a solution of 5-hydroxy-3H-isobenzofuran-1-one (2.00 g, 13.3 mmol) and sodium 2-chloro-2,2-difluoroacetate (6.09 g, 40.0 mmol) in DMF (20 mL) was added K₂CO₃ (5.52 g, 40.0 mmol), then the mixture was stirred at 100° C. for 48 hours. The mixture was cooled to rt, poured into a saturated solution of NH₄Cl (20 mL) and extracted with EtOAc (20 mL×3). The combined organic layer was washed with brine, dried over MgSO₄, filtered and concentrated in vacuo. The residue was purified by silica gel chromatography (EtOAc/petroleum ether=0 to 15%) to give 5-(difluoromethoxy)-3H-isobenzofuran-1-one as a white solid (1.83 g, 28% yield). LCMS ESI m/z 201 (M+H)⁺. ¹H NMR (400 MHz, CDCl₃) δ 7.94 (d, J=8.4 Hz, 1H), 7.28 (dd, J=8.5 Hz, 1H), 7.24 (s, 1H), 6.63 (t, J=72.4 Hz, 1H), 5.32 (s, 2H).

### Step 2

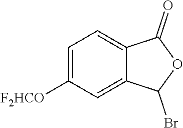 [SMILES: O=C1OC(Br)c2cc(OC(F)F)ccc21]

### 3-Bromo-5-(difluoromethoxy)isobenzofuran-1(3H)-one

A mixture of 5-(difluoromethoxy)-3H-isobenzofuran-1-one (750 mg, 3.75 mmol), NBS (801 mg, 4.5 mmol) and AIBN (62 mg, 3.75 mmol) in CCl₄ (10 mL) was heated to 80° C. and stirred for 16 hours. The mixture was cooled to rt, diluted with H₂O (30 mL) and extracted with DCM (30 mL×3). The combined organic layer was dried over anhydrous Na₂SO₄, filtered and concentrated in vacuo. The residue was purified by silica gel chromatography (EtOAc/petroleum ether=0 to 9%) to obtain 3-bromo-5-(difluoromethoxy)-3H-isobenzofuran-1-one as a white solid (580 mg, 55%). LCMS ESI m/z 279 (M+H)⁺.

### Step 3

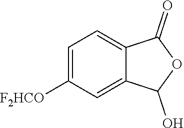 [SMILES: O=C1OC(O)c2cc(OC(F)F)ccc21]

### 5-(Difluoromethoxy)-3-hydroxyisobenzofuran-1(3H)-one

A solution of 3-bromo-5-(difluoromethoxy)-3H-isobenzofuran-1-one (580 mg, 2.08 mmol) in H₂O (25 mL) was stirred at 100° C. for 3 hours. The reaction was cooled to rt and extracted with DCM (50 mL×3). The combined organic layer was dried over anhydrous Na₂SO₄, filtered and concentrated in vacuo to give 5-(difluoromethoxy)-3-hydroxy-3H-isobenzofuran-1-one as a light yellow oil (390 mg, 87/o yield). LCMS ESI m/z 217 (M+H)⁺.

### Step 4

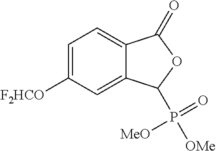 [SMILES: COP(=O)(OC)C1OC(=O)c2ccc(OC(F)F)cc21]

### Dimethyl (6-(difluoromethoxy)-3-oxo-1,3-dihydroisobenzofuran-1-yl)phosphonate

A solution of 5-(difluoromethoxy)-3-hydroxy-3H-isobenzofuran-1-one (390 mg, 1.80 mmol) in dimethylphosphite (30 mL) was stirred at 100° C. for 2 hours. The mixture was cooled to rt, diluted with H₂O (10 mL) and extracted with EtOAc (20 mL×3). The combined organic layer was washed with water and brine, dried over anhydrous Na₂SO₄, filtered and concentrated in vacuo to give dimethyl (6-(difluoromethoxy)-3-oxo-1,3-dihydroisobenzofuran-1-yl)phosphonate as a light yellow oil (230 mg, 41% yield). This was used without further purification. LCMS ESI m/z 309 (M+H)⁺.

### Step 5

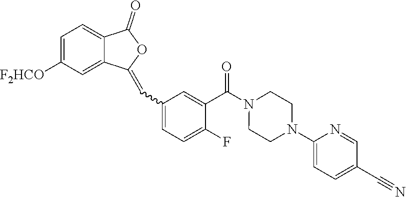 [SMILES: N#Cc1ccc(N2CCN(C(=O)c3cc(C=C4OC(=O)c5ccc(OC(F)F)cc54)ccc3F)CC2)nc1]

### 6-(4-(5-((6-(Difluoromethoxy)-3-oxoisobenzofuran-1(3H)-ylidene)methyl)-2-fluorobenzoyl)piperazin-1-yl)nicotinonitrile

To a solution of (6-(difluoromethoxy)-3-oxo-1,3-dihydroisobenzofuran-1-yl)phosphonate (230 mg, 0.75 mmol) in THF (dry, 5 mL) under Ar (g), was added 6-(4-(2-fluoro-5-formylbenzoyl)piperazin-1-yl)nicotinonitrile (303 mg, 0.90 mmol) and triethylamine (197 mg, 1.95 mmol). The reaction mixture was stirred at rt for 12 hours, then poured into water (10 mL) and extracted with EtOAc (20 mL×3). The combined organic layer was washed with brine, dried over Na₂SO₄, filtered and concentrated in vacuo to give 6-[4-[5-[6-(difluoromethoxy)-3-oxo-isobenzofuran-1-ylidene]methyl]-2-fluoro-benzoyl]piperazin-1-yl]pyridine-3-carbonitrile as a yellow solid (397 mg, 41% yield). LCMS ESI m/z 521 (M+H)⁺.

### Step 6

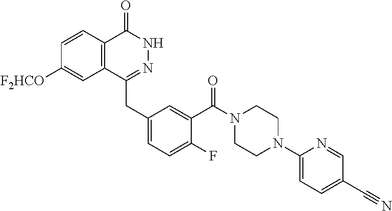 [SMILES: N#Cc1ccc(N2CCN(C(=O)c3cc(Cc4n[nH]c(=O)c5ccc(OC(F)F)cc45)ccc3F)CC2)nc1]

### 6-(4-(5-((7-(Difluoromethoxy)-4-oxo-3,4-dihydrophthalazin-1-yl)methyl)-2-fluorobenzoyl)piperazin-1-yl)nicotinonitrile

To a solution of 6-[4-[5-[6-(difluoromethoxy)-3-oxo-isobenzofuran-1-ylidene]methyl]-2-fluoro-benzoyl]piperazin-1-yl]pyridine-3-carbonitrile (397 mg, 0.63 mmol) in THF (10 mL) was added N₂H₄·H₂O (655 mg, 11.14 mmol), and the mixture was stirred at 70° C. for 4 hours. The solids were filtered, washed with water (5 mL×2), and dried under vacuum to give 6-(4-(5-((7-(difluoromethoxy)-4-oxo-3,4-dihydrophthalazin-1-yl)methyl)-2-fluorobenzoyl)piperazin-1-yl)nicotinonitrile as a white solid (260 mg, 44% yield). LCMS ESI m/z 535 (M+H)⁺. ¹H NMR (400 MHz, DMSO-d₆) δ 12.66 (s, 1H), 8.51 (d, J=2.2 Hz, 1H), 8.32 (d, J=8.6 Hz, 1H), 7.90 (dd, J=9.1, 2.3 Hz, 1H), 7.69-7.60 (m, 2H), 7.51-7.21 (m, 4H), 6.92 (d, J=9.1 Hz, 1H), 4.33 (s, 2H), 3.77-3.73 (m, 4H), 3.61 (m, 2H), 3.31 m, 2H).

### Synthesis of Example 22: 6-(4-(2-Fluoro-5-((4-oxo-7-((2,2,2-trifluoroethyl)amino)-3,4-dihydrophthalazin-1-yl)methyl)benzoyl)piperazin-1-yl)nicotinonitrile

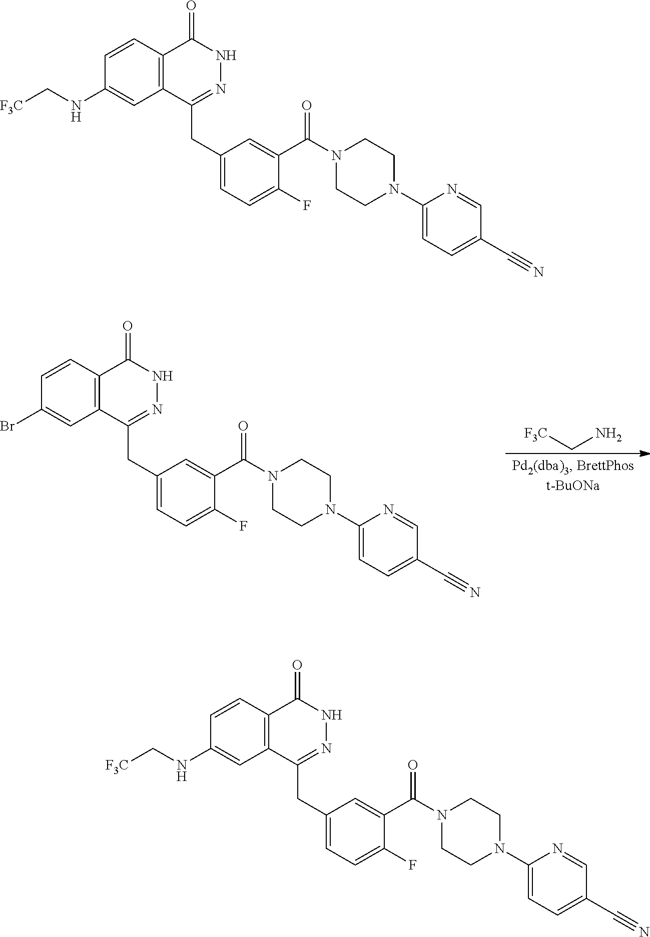 [SMILES: N#Cc1ccc(N2CCN(C(=O)c3cc(Cc4n[nH]c(=O)c5ccc(NCC(F)(F)F)cc45)ccc3F)CC2)nc1 ; N#Cc1ccc(N2CCN(C(=O)c3cc(Cc4n[nH]c(=O)c5ccc(Br)cc45)ccc3F)CC2)nc1 ; NCC(F)(F)F ; N#Cc1ccc(N2CCN(C(=O)c3cc(Cc4n[nH]c(=O)c5ccc(NCC(F)(F)F)cc45)ccc3F)CC2)nc1]

### Step 1

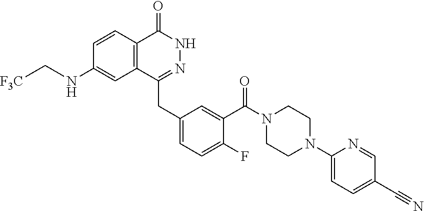 [SMILES: N#Cc1ccc(N2CCN(C(=O)c3cc(Cc4n[nH]c(=O)c5ccc(NCC(F)(F)F)cc45)ccc3F)CC2)nc1]

### 6-(4-(2-Fluoro-5-((4-oxo-7-((2,2,2-trifluoroethyl)amino)-3,4-dihydrophthalazin-1-yl)methyl)benzoyl)piperazin-1-yl)nicotinonitrile

A mixture of 6-(4-(5-((7-bromo-4-oxo-3,4-dihydrophthalazin-1-yl)methyl)-2-fluorobenzoyl)piperazin-1-yl)nicotinonitrile (200 mg, 0.36 mmol), 2,2,2-trifluoroethan-1-amine (108 mg, 1.10 mmol), Pd₂(dba). (20 mg, 0.02 mmol), XPhos (17 mg, 0.04 mmol) and sodium tert-butoxide (105 mg, 1.10 mmol) in DMA (5 mL) was stirred at 130° C. for 2 hours. The mixture was cooled to rt, filtered and the filtrate was purified by prep-HPLC to give 6-(4-(2-fluoro-5-((4-oxo-7-((2,2,2-trifluoroethyl)amino)-3,4-dihydrophthalazin-1-yl)methyl)benzoyl) piperazin-1-yl)nicotinonitrile as a white solid (19 mg, 9% yield). LCMS ESI m/z 566 (M+H)⁺. ¹H NMR (400 MHz, DMSO-d₆) δ 12.17 (s, 1H), 8.51 (d, J=2.2 Hz, 1H), 7.97 (d, J=8.8 Hz, 1H), 7.90 (dd, J=9.1, 2.3 Hz, 1H), 7.50-7.42 (m, 1H), 7.36 (dd, J=6.5, 2.0 Hz, 1H), 7.30-7.15 (m, 3H), 7.02 (d, J=1.8 Hz, 1H), 6.91 (d, J=9.1 Hz, 1H), 4.23 (s, 2H), 4.20-4.08 (m, 2H), 3.74 (m, 4H), 3.59-3.51 (m, 2H), 3.31-3.26 (m, 2H).

### Synthesis of Example 23: 6-(4-(2-Fluoro-5-((7-(6-hydroxy-2-azaspiro[3.3]heptan-2-yl)-4-oxo-3,4-dihydrophthalazin-1-yl)methyl)benzoyl)piperazin-1-yl)nicotinonitrile

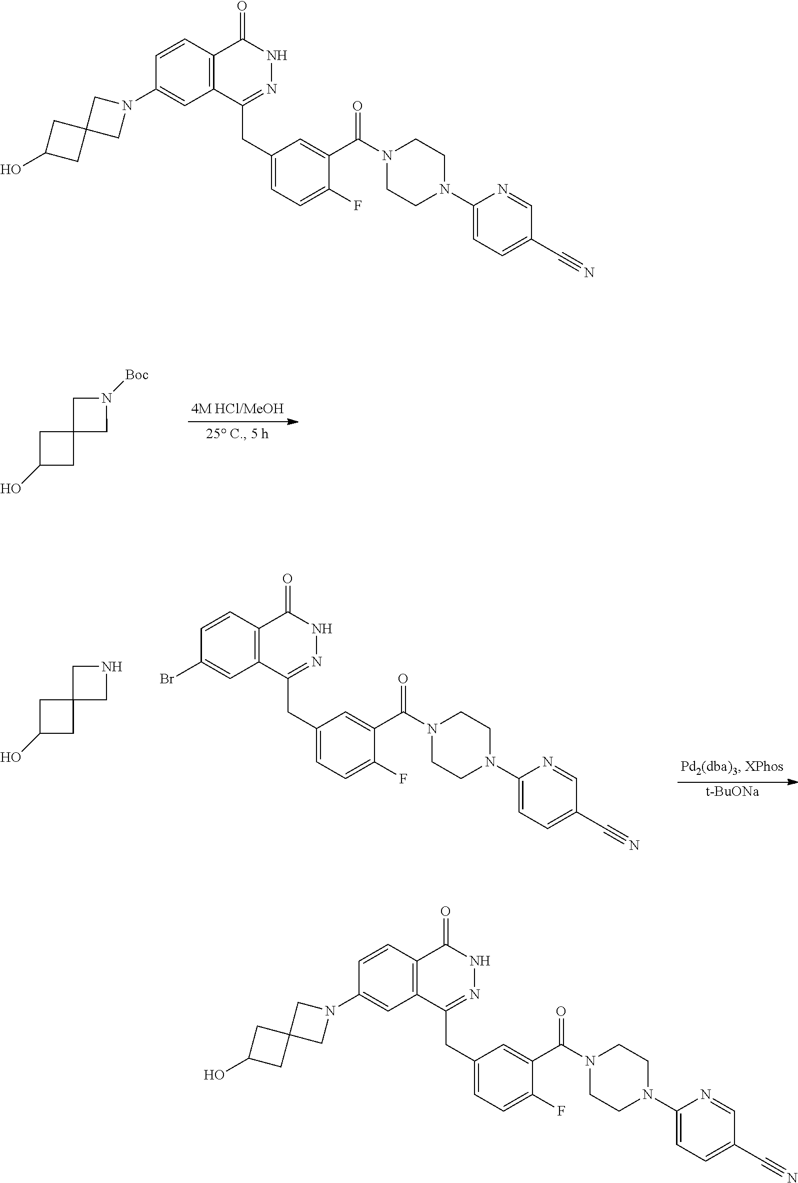 [SMILES: N#Cc1ccc(N2CCN(C(=O)c3cc(Cc4n[nH]c(=O)c5ccc(N6CC7(CC(O)C7)C6)cc45)ccc3F)CC2)nc1 ; CC(C)(C)OC(=O)N1CC2(CC(O)C2)C1 ; "t-BuONa" ; OC1CC2(CNC2)C1 ; N#Cc1ccc(N2CCN(C(=O)c3cc(Cc4n[nH]c(=O)c5ccc(Br)cc45)ccc3F)CC2)nc1 ; N#Cc1ccc(N2CCN(C(=O)c3cc(Cc4n[nH]c(=O)c5ccc(N6CC7(CC(O)C7)C6)cc45)ccc3F)CC2)nc1]

### Step 1

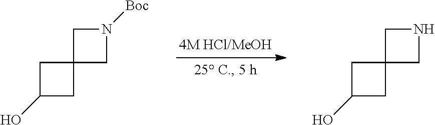 [SMILES: CC(C)(C)OC(=O)N1CC2(CC(O)C2)C1 ; OC1CC2(CNC2)C1]

### 2-Azaspiro[3.3]heptan-6-ol hydrochloride

To a solution of tert-butyl 6-hydroxy-2-azaspiro[3.3]heptane-2-carboxylate (200 mg, 937 μmol) in HCl/MeOH (4 M, 4 mL) at 25° C. The reaction mixture was stirred at 25° C. for 5 hours. The mixture was concentrated to give 2-azaspiro[3.3]heptan-6-ol hydrochloride (100 mg, 71% yield) as a white oil.

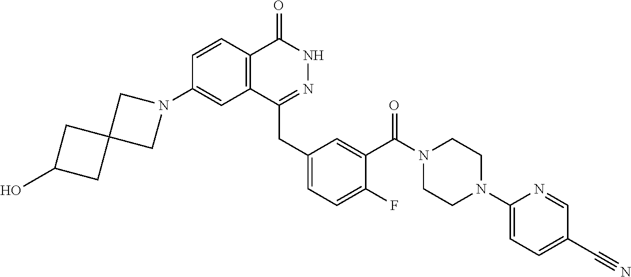 [SMILES: N#Cc1ccc(N2CCN(C(=O)c3cc(Cc4n[nH]c(=O)c5ccc(N6CC7(CC(O)C7)C6)cc45)ccc3F)CC2)nc1]

### 6-(4-(2-Fluoro-5-((7-(6-hydroxy-2-azaspiro[3.3]heptan-2-yl)-4-oxo-3,4-dihydrophthalazin-1-yl)methyl)benzoyl)piperazin-1-yl)nicotinonitrile

To a solution of 6-[4-[5-[(7-bromo-4-oxo-3H-phthalazin-1-yl)methyl]-2-fluoro-benzoyl]piperazin-1-yl]pyridine-3-carbonitrile (150 mg, 274 μmol) in DMF (4 mL) was added 2-azaspiro[3.3]heptan-6-ol hydrochloride (62.0 mg, 548 μmol), t-BuOK (185 mg, 1.64 mmol), X-Phos (26.1 mg, 54.8 μmol) and Pd₂(dba)₃ (15.8 mg, 27.4 μmol) at 25° C. under N₂ (g). The reaction was stirred at 110° C. under microwave for 45 min. The mixture was filtered and purified by Pre-HPLC to give 6-(4-(2-fluoro-5-((7-(6-hydroxy-2-azaspiro[3.3]heptan-2-yl)-4-oxo-3,4-dihydrophthalazin-1-yl)methyl)benzoyl)piperazin-1-yl)nicotinonitrile (13.5 mg, 9% yield) as a white solid. LCMS ESI m/z 580.3 [M+H]⁺. ¹H NMR (400 MHz, DMSO-d₆) δ 12.14 (s, 1H), 8.52 (d, J=2.3 Hz, 1H), 7.99 (d, J=8.7 Hz, 1H), 7.90 (dd, J=9.1, 2.4 Hz, 1H), 7.41 (t, J=6.5 Hz, 2H), 7.25 (t, J=9.2 Hz, 1H), 6.92 (d, J=9.0 Hz, 1H), 6.79 (dd, J=8.7, 2.2 Hz, 1H), 6.51 (d, J=2.1 Hz, 1H), 5.07 (d, J=6.1 Hz, H), 4.21 (s, 2H), 4.01 (dd, J=13.4, 7.0 Hz, 1H), 3.89 (d, J=17.3 Hz, 4H), 3.75 (m, 4H), 3.62 (m, 2H), 2.46 (dd, J=7.6, 4.9 Hz, 4H), 2.07-1.97 (m, 2H).

### Synthesis of Example 24: 6-(4-(2-Fluoro-5-((4-oxo-7-(3,3,3-trifluoroprop-1-ynyl)-3,4-dihydrophthalazin-1-yl)methyl)benzoyl)piperazin-1-yl)nicotinonitrile

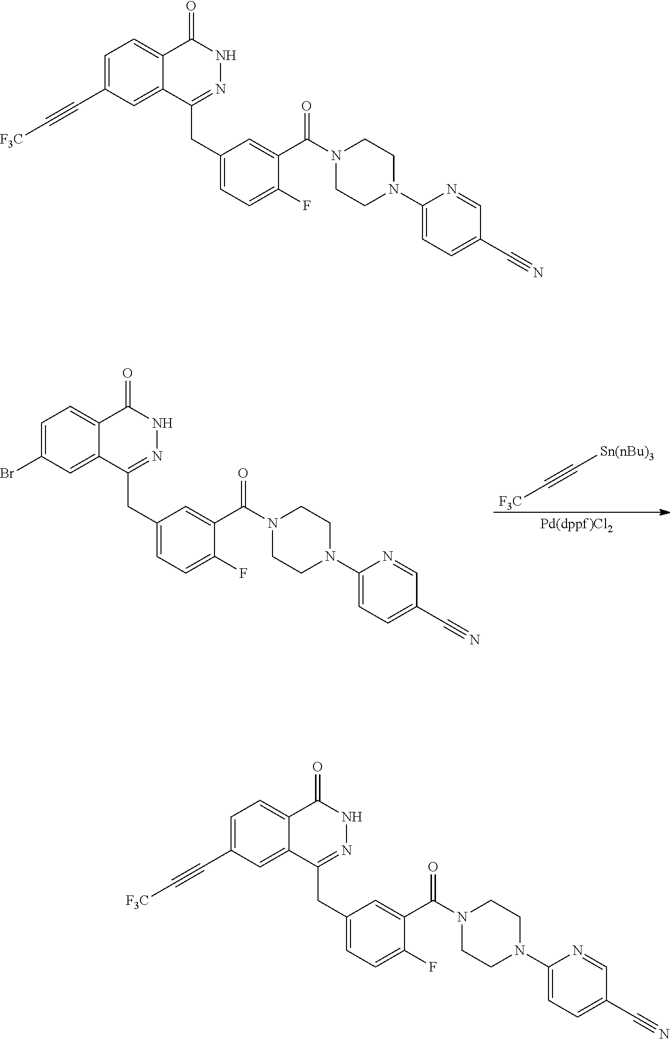

### Step 1

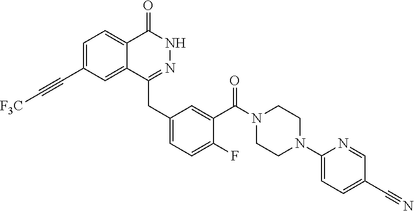 [SMILES: N#Cc1ccc(N2CCN(C(=O)c3cc(Cc4n[nH]c(=O)c5ccc(C#CC(F)(F)F)cc45)ccc3F)CC2)nc1]

### 6-(4-(2-Fluoro-5-((4-oxo-7-(3,3,3-trifluoroprop-1-ynyl)-3,4-dihydrophthalazin-1-yl)methyl)benzoyl)piperazin-1-yl)nicotinonitrile

A mixture of 6-(4-(5-((7-bromo-4-oxo-3,4-dihydrophthalazin-1-yl)methyl)-2-fluorobenzoyl)piperazin-1-yl)nicotinonitrile (200 mg, 0.37 mmol), tributyl(3,3,3-trifluoroprop-1-ynyl)stannane (422 mg, 1.10 mmol) and Pd(dppf)Cl₂ (58 mg, 0.08 mmol) in 1,4-dioxane (10 mL) were stirred at 100° C. under Ar (g) for 14 hours. The mixture was cooled to rt, diluted with water (20 mL) and extracted with EtOAc (3×20 mL). The combined organic layer was dried over Na₂SO₄, filtered and concentrated in vacuo. The residue was purified by silica gel chromatography (EtOAc/petroleum ether=80% to 100%) to give 6-(4-(2-fluoro-5-((4-oxo-7-(3,3,3-trifluoroprop-1-ynyl)-3,4-dihydrophthalazin-1-yl)methyl)benzoyl)piperazin-1-yl)nicotinonitrile as a white solid (35 mg, 17% yield). LCMS ESI m/z 561 (M+H)⁺. ¹H NMR (400 MHz, DMSO-d₆) δ 12.81 (s, 1H), 8.53-8.45 (m, 2H), 8.32 (d, J=8.2 Hz, 1H), 8.12 (d, J=9.4 Hz, H), 7.90 (dd, J=9.1, 2.4 Hz, 1H), 7.46-7.27 (m, 3H), 6.92 (d, J=9.1 Hz, 1H), 4.38 (s, 2H), 3.80-3.70 (m, 4H), 3.65-3.58 (m, 2H), 3.32-3.29 (m, 2H).

### Synthesis of Example 25: 6-(4-(2-Fluoro-5-((4-oxo-7-(3,3,3-trifluoropropyl)-3,4-dihydrophthalazin-1-yl)methyl)benzoyl)piperazin-1-yl)nicotinonitrile

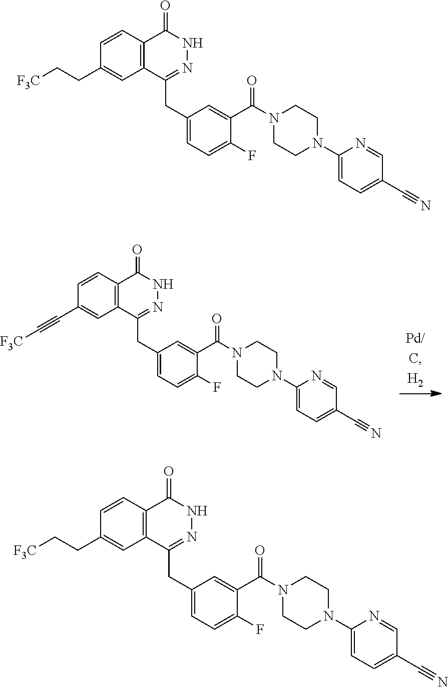 [SMILES: N#Cc1ccc(N2CCN(C(=O)c3cc(Cc4n[nH]c(=O)c5ccc(CCC(F)(F)F)cc45)ccc3F)CC2)nc1 ; N#Cc1ccc(N2CCN(C(=O)c3cc(Cc4n[nH]c(=O)c5ccc(C#CC(F)(F)F)cc45)ccc3F)CC2)nc1 ; N#Cc1ccc(N2CCN(C(=O)c3cc(Cc4n[nH]c(=O)c5ccc(CCC(F)(F)F)cc45)ccc3F)CC2)nc1]

### Step 1

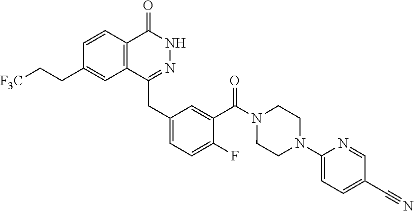 [SMILES: N#Cc1ccc(N2CCN(C(=O)c3cc(Cc4n[nH]c(=O)c5ccc(CCC(F)(F)F)cc45)ccc3F)CC2)nc1]

### 6-(4-(2-Fluoro-5-((4-oxo-7-(3,3,3-trifluoropropyl)-3,4-dihydrophthalazin-1-yl)methyl)benzoyl)piperazin-1-yl)nicotinonitrile

A mixture of 6-(4-(2-fluoro-5-((4-oxo-7-(3,3,3-trifluoroprop-1-ynyl)-3,4-dihydrophthalazin-1-yl)methyl)benzoyl)piperazin-1-yl)nicotinonitrile (22 mg, 0.04 mmol) and Pd/C (10%, 4 mg) in MeOH (2 mL) were stirred at rt for 14 hours under H₂(g). The mixture was filtered and concentrated in vacuo. The residue was purified by prep-HPLC to afford 6-(4-(2-fluoro-5-((4-oxo-7-(3,3,3-trifluoropropyl)-3,4-dihydrophthalazin-1-yl)methyl)benzoyl)piperazin-1-yl)nicotinonitrile as a white solid (10 mg, 45% yield). LCMS ESI m/z 561 (M+H)⁺. ¹H NMR (400 MHz, CDCl₃) δ 10.04 (s, 1H), 8.45-8.38 (m, 2H), 7.68-7.59 (m, 2H), 7.53 (s, 1H), 7.39-7.31 (m, 2H), 7.08 (t, J=8.8 Hz, 1H), 6.61 (d, J=8.9 Hz, 1H), 4.28 (s, 2H), 3.89 (m, 2H), 3.77 (m, 2H), 3.69 (t, J=4.9 Hz, 2H), 3.43 (m, 2H), 3.03 (t, J=7.6 Hz, 2H), 2.51-2.35 (m, 2H).

### Synthesis of Example 26: 6-(4-(2-Fluoro-5-((4-oxo-7-(prop-2-yn-1-yloxy)-3,4-dihydrophthalazin-1-yl)methyl)benzoyl)piperazin-1-yl)nicotinonitrile

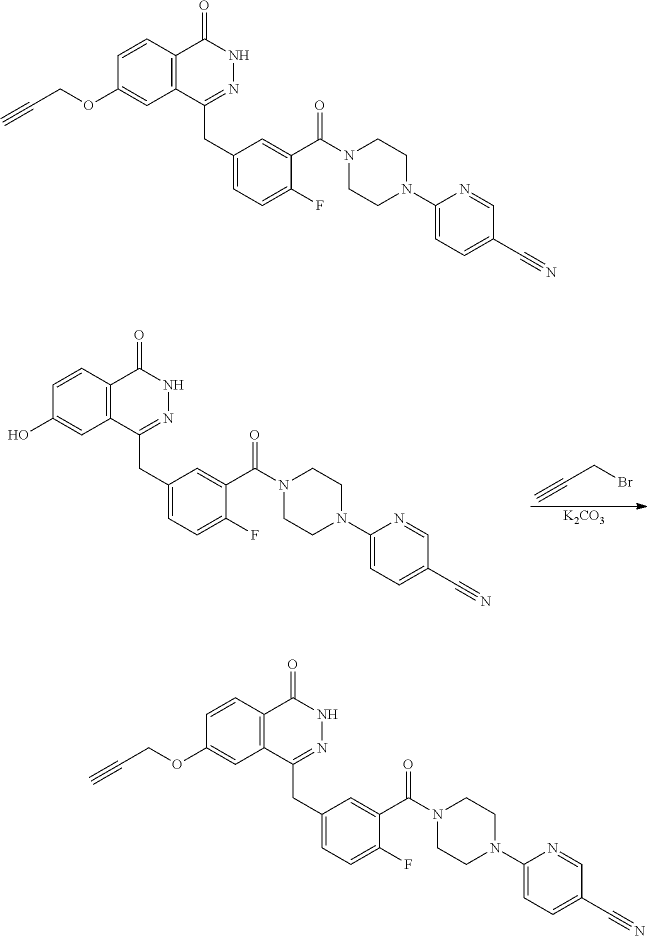

### Step 1

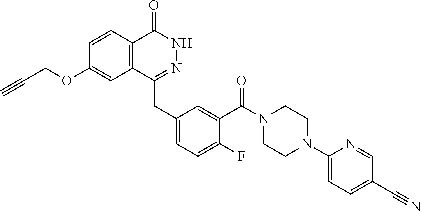 [SMILES: C#CCOc1ccc2c(=O)[nH]nc(Cc3ccc(F)c(C(=O)N4CCN(c5ccc(C#N)cn5)CC4)c3)c2c1]

### 6-(4-(2-Fluoro-5-((4-oxo-7-(prop-2-yn-1-yloxy)-3,4-dihydrophthalazin-1-yl)methyl)benzoyl)piperazin-1-yl)nicotinonitrile

A mixture of 6-(4-(2-fluoro-5-((4-oxo-7-(prop-2-yn-1-yloxy)-3,4-dihydrophthalazin-1-yl)methyl)benzoyl)piperazin-1-yl)nicotinonitrile (100 mg, 0.21 mmol), 3-bromoprop-1-yne (26 mg, 0.22 mmol) and K₂CO₃ (86 mg, 0.63 mmol) in DMF (5 mL) was stirred at 50° C. for 1 hour. The mixture was cooled to rt, diluted with H₂O (10 mL) and extracted with EtOAc (20 mL×3). The combined organic layer was concentrated in vacuo and the residue was purified by prep-HPLC to give 64-(2-fluoro-5-((4-oxo-7-(prop-2-yn-1-yloxy)-3,4-dihydrophthalazin-1-yl)methyl) benzoyl)piperazin-1-yl)nicotinonitrile as a white solid (40 mg, 37% yield). LCMS ESI m/z 523 [M+H]⁺. ¹H NMR (40) MHz, CDCl₃) δ 9.95 (s, 1H), 8.34 (dd, J=10.0, 5.5 Hz, 2H), 7.59 (dd, J=9.0, 2.3 Hz, 1H), 7.29 (dd, J=12.8, 4.1 Hz, 3H), 7.14 (d, J=2.4 Hz, 1H), 7.00 (t, J=8.7 Hz, 1H), 6.55 (d, J=9.0 Hz, 1H), 4.71 (d, J=2.4 Hz, 2H), 4.18 (s, 2H), 3.82 (m, 2H), 3.71 (m, 2H), 3.62 (m, 2H), 3.36 (s, 2H), 2.58 (t, J=2.4 Hz, 1H).

### Synthesis of Example 27: (E)-6-(4-(2-Fluoro-5-((4-oxo-7-(3,3,3-trifluoroprop-1-enyl)-3,4-dihydrophthalazin-1-yl)methyl)benzoyl)piperazin-1-yl)nicotinonitrile

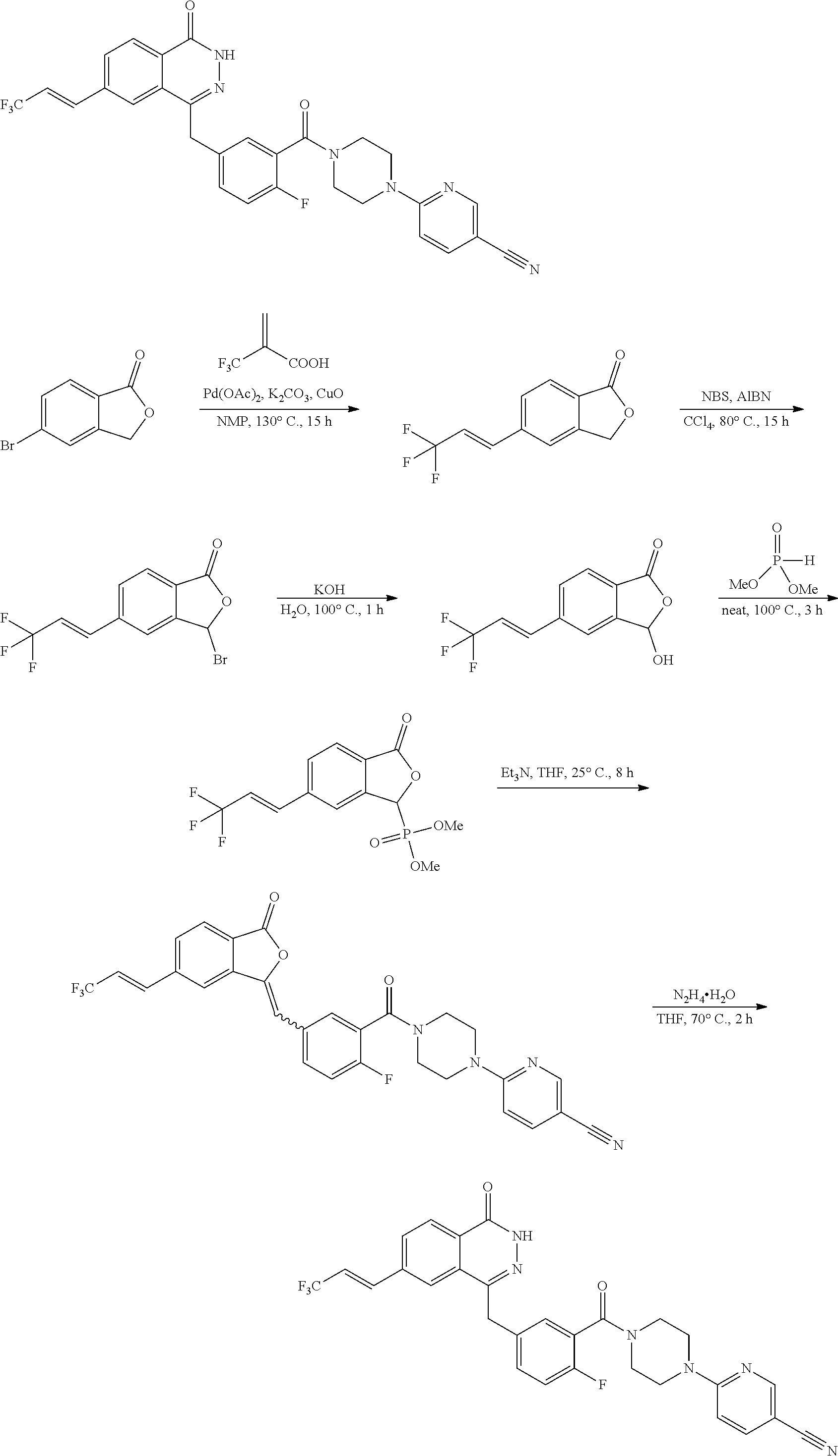

### Step 1

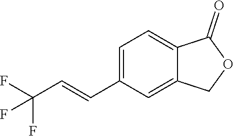 [SMILES: O=C1OCc2cc(/C=C/C(F)(F)F)ccc21]

### (E)-5-(3,3,3-Trifluoroprop-1-en-1-yl)isobenzofuran-1(3H)-one

To a solution of 5-bromoisobenzofuran-1(3H)-one (4.5 g, 21.12 mmol) in NMP (50 mL) was added 2-(trifluoromethyl)prop-2-enoic acid (4.14 g, 29.57 mmol), Pd(OAc)₂ (474 mg, 2.11 mmol) and CuO (1.70 g, 21.1 mmol) at 25° C. under N₂ (g). The reaction mixture was stirred at 130° C. for 15 hours. Water (20 mL) and EtOAc (30 mL) were added to the mixture. The organic layer was separated, washed with brine, dried over Na₂SO₄ and concentrated to give a residue. The residue was purified by silica gel chromatography (petroleum ether/EtOAc=20/1 to 2/1) to give (E)-5-(3,3,3-trifluoroprop-1-en-1-yl)isobenzofuran-1(3H)-one (1.6 g, 33.2% yield) as a white solid. ¹H NMR (400 MHz, CDCl₃) δ 7.95 (d, J=8.0 Hz, 1H), 7.63 (d, J=8.0 Hz, 1H), 7.57 (s, 1H), 7.29-7.22 (m, 1H), 6.36 (dq, J=16.1, 6.3 Hz, 1H), 5.35 (s, 2H).

### Step 2

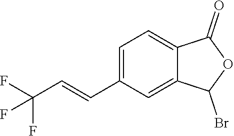 [SMILES: O=C1OC(Br)c2cc(/C=C/C(F)(F)F)ccc21]

### (E)-3-Bromo-5-(3,3,3-trifluoroprop-1-en-1-yl)isobenzofuran-1(3H)-one

To a solution of (E)-5-(3,3,3-trifluoroprop-1-en-1-yl)isobenzofuran-1(3H)-one (1.00 g, 4.38 mmol) in CCl₄ (10 mL) was added 1-bromopyrrolidine-2,5-dione (1.17 g, 6.57 mmol, 557.72 μL), AIBN (72.0 mg, 438 μmol) at 25° C. under N₂ (g). The reaction mixture was stirred at 80° C. for 15 hours. Water (20 mL) and EtOAc (30 mL) were added to the mixture. The organic layer was separated, washed with brine, dried over Na₂SO₄ and concentrated to give a residue. The residue was purified by silica gel chromatography (petroleum ether/EtOAc=20/1 to 0/1) to give (E)-3-bromo-5-(3,3,3-trifluoroprop-1-en-1-yl)isobenzofuran-1(3H)-one (1.00 g, 74% yield) as a white solid.

### Step 3

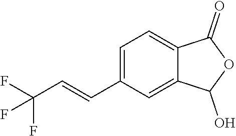 [SMILES: O=C1OC(O)c2cc(/C=C/C(F)(F)F)ccc21]

### (E)-3-Hydroxy-5-(3,3,3-trifluoroprop-1-en-1-yl)isobenzofuran-1(3H)-one

A solution of (E)-3-bromo-5-(3,3,3-trifluoroprop-1-en-1-yl)isobenzofuran-1(3H)-one (1.5 g, 4.88 mmol) in 1 mol/L KOH (15 mL) was stirred at 100° C. for 1 hour. The mixture was acidified with 1N HCl until the solution reached pH 2-3. The solids were collected by filtration, washed with water and dried under vacuum to give (E)-3-hydroxy-5-(3,3,3-trifluoroprop-1-en-1-yl)isobenzofuran-1(3H)-one (1 g, 84% yield) as a white solid. ¹H NMR (400 MHz, DMSO-d₆) δ 8.27 (d, J=8.5 Hz, 1H), 8.04 (s, 1H), 7.96 (d, J=8.0 Hz, 1H), 7.87 (d, J=7.9 Hz, 1H), 7.54 (dd, J=16.2, 1.9 Hz, 1H), 7.15-6.99 (m, 1H), 6.70 (d, J=8.3 Hz, 1H).

### Step 4

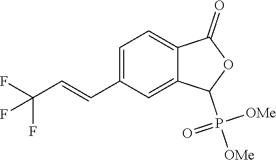 [SMILES: COP(=O)(OC)C1OC(=O)c2ccc(/C=C/C(F)(F)F)cc21]

### Dimethyl (E)-(3-oxo-6-(3,3,3-trifluoroprop-1-en-1-yl)-1,3-dihydroisobenzofuran-1-yl)phosphonate

A solution of (E)-3-hydroxy-5-(3,3,3-trifluoroprop-1-en-1-yl)isobenzofuran-1(3H)-one (1.00 g, 4.10 mmol) in dimethylphosphite (4 mL) was stirred at 100° C. for 3 hours. Water (20 mL) and EtOAc (30 mL) were added to the mixture. The organic layer was separated, washed with brine, dried over Na₂SO₄, and concentrated to give a residue. The residue was purified by silica gel chromatography (petroleum ether/EtOAc=20/1 to 1/2) to give dimethyl (E)-(3-oxo-6-(3,3,3-trifluoroprop-1-en-1-yl)-1,3-dihydroisobenzofuran-1-yl)phosphonate (750 mg, 55% yield) as a white solid. ¹H NMR (400 MHz, DMSO-d₆) δ 8.06-7.92 (m, 3H), 7.61 (dd, J=16.3, 2.1 Hz, 1H), 7.07 (dq, J=16.2, 6.9 Hz, 1H), 6.37 (d, J=11.2 Hz, 1H), 3.82 (d, J=10.9 Hz, 3H), 3.65 (d, J=10.7 Hz, 3H).

### Step 5

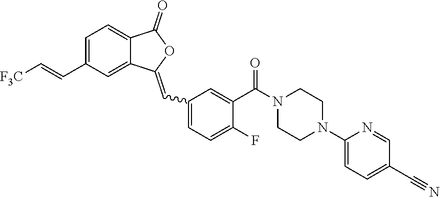 [SMILES: N#Cc1ccc(N2CCN(C(=O)c3cc(C=C4OC(=O)c5ccc(/C=C/C(F)(F)F)cc54)ccc3F)CC2)nc1]

### 6-(4-(2-Fluoro-5-(((Z)-3-oxo-6-((E)-3,3,3-trifluoroprop-1-en-1-yl)isobenzofuran-1(3H)-ylidene)methyl)benzoyl)piperazin-1-yl)nicotinonitrile

To a solution of (E)-(3-oxo-6-(3,3,3-trifluoroprop-1-en-1-yl)-1,3-dihydroisobenzofuran-1-yl)phosphonate (300 mg, 892.33 μmol) in THF (4 mL) was added 6-[4-(2-fluoro-5-formyl-benzoyl)piperazin-1-yl]pyridine-3-carbonitrile (305 mg, 901 μmol), Et₃N (271 mg, 2.68 mmol, 373 μL) at 25° C. The reaction mixture was stirred at 25° C. for 8 hours. Water (10 mL) and EtOAc (10 mL) were added to the mixture, the organic layer was separated, washed with brine, dried over Na₂SO₄ and concentrated to give 6-(4-(2-fluoro-5-(((Z)-3-oxo-6-((E)-3,3,3-trifluoroprop-1-en-1-yl)isobenzofuran-1(3H)-ylidene)methyl)benzoyl)piperazin-1-yl)nicotinonitrile (280 mg crude, 57% yield) as a white solid.

### Step 6

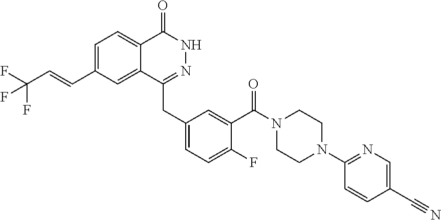 [SMILES: N#Cc1ccc(N2CCN(C(=O)c3cc(Cc4n[nH]c(=O)c5ccc(/C=C/C(F)(F)F)cc45)ccc3F)CC2)nc1]

### (E)-6-(4-(2-Fluoro-5-((4-oxo-7-(3,3,3-trifluoroprop-1-en-1-yl)-3,4-dihydrophthalazin-1-yl)methyl)benzoyl)piperazin-1-yl)nicotinonitrile

To a solution of 6-(4-(2-fluoro-5-(((Z)-3-oxo-6-((E)-3,3,3-trifluoroprop-1-en-1-yl)isobenzofuran-1(3H)-ylidene)methyl)benzoyl)piperazin-1-yl)nicotinonitrile (280 mg, 511 μmol) in THF (2 mL) was added hydrazine hydrate (2.05 g, 32.8 mmol, 2 mL, 80% purity) at 25° C. under N₂ (g). The reaction mixture was stirred at 70° C. for 2 hours. The mixture was filtered and purified by prep-HPLC to give (E)-6-(4-(2-fluoro-5-((4-oxo-7-(3,3,3-trifluoroprop-1-en-1-yl)-3,4-dihydrophthalazin-1-yl)methyl)benzoyl)piperazin-1-yl)nicotinonitrile (49.8 mg, 17% yield) as a white solid. LCMS ESI m/z 562.5 [M+H]⁺. ¹H NMR (400 MHz, DMSO-d₆) δ 12.66 (s, 1H), 8.51 (d, J=1.9 Hz, 1H), 8.33 (s, 1H), 8.28 (d, J=8.3 Hz, 1H), 8.16 (dd, J=8.3, 1.2 Hz, 1H), 7.89 (dd, J=9.1, 2.4 Hz, 1H), 7.56 (dd, J=16.3, 2.1 Hz, 1H), 7.50 (dd, J=10.5, 4.1 Hz, 2H), 7.27 (dd, J=12.6, 6.0 Hz, 1H), 7.15-7.02 (m, 1H), 6.91 (d, J=9.1 Hz, 1H), 4.36 (s, 2H), 3.75 (m, 4H), 3.59 (m, 2H), 3.30 (m, 2H).

### Synthesis of Example 28: 6-(4-(5-((7-(Dimethylamino)-4-oxo-3,4-dihydrophthalazin-1-yl)methyl)-2-fluorobenzoyl)piperazin-1-yl)nicotinonitrile

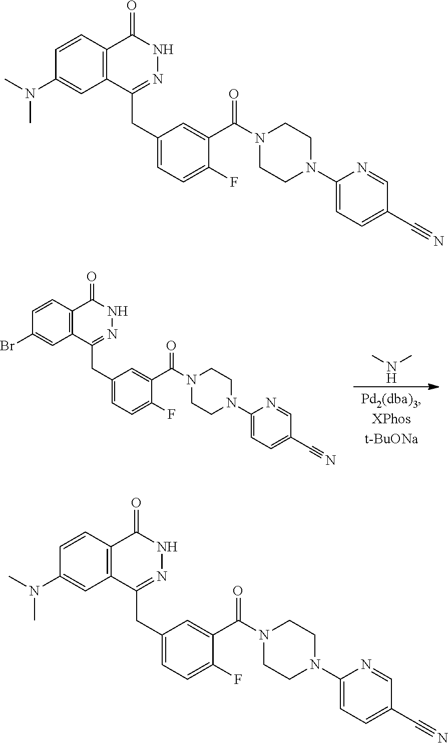

### Step 1

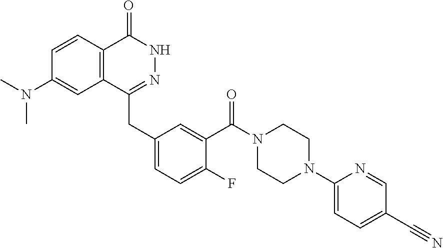 [SMILES: CN(C)c1ccc2c(=O)[nH]nc(Cc3ccc(F)c(C(=O)N4CCN(c5ccc(C#N)cn5)CC4)c3)c2c1]

### 6-(4-(5-((7-(dimethylamino)-4-oxo-3,4-dihydrophthalazin-1-yl)methyl)-2-fluorobenzoyl)piperazin-1-yl)nicotinonitrile

To a solution of 6-(4-(5-((7-bromo-4-oxo-3,4-dihydrophthalazin-1-yl)methyl)-2-fluorobenzoyl)piperazin-1-yl)nicotinonitrile (100 mg, 183 μmol) in DMA (3 mL) was added dimethylamine (33.0 mg, 731 μmol), t-BuONa (44.0 mg, 457 μmol), Pd₂(dba)₃ (1.67 mg, 1.83 μmol), and XPhos (1.74 mg, 3.65 μmol). The reaction mixture was heated to 110° C. for 16 hours. The mixture was filtered and concentrated to the crude. The crude was purified by prep-HPLC to give 6-(4-(5-((7-(dimethylamino)-4-oxo-3,4-dihydrophthalazin-1-yl)methyl)-2-fluorobenzoyl)piperazin-1-yl)nicotinonitrile (30 mg, 31% yield) as a white solid. LCMS ESI m/z 511.7 [M+H]⁺. ¹H NMR (400 MHz, DMSO-d₆) δ 12.13 (s, 1H), 8.52 (d, J=2.3 Hz, 1H), 8.01 (d, J=9.0 Hz, 1H), 7.90 (dd, J=9.1, 2.3 Hz, 1H), 7.44 (t, J=5.0 Hz, 2H), 7.28-7.16 (m, 2H), 6.93 (d, J=9.1 Hz, 1H), 6.76 (d, J=2.3 Hz, 1H), 4.27 (s, 2H), 3.75 (m, 4H), 3.61 (s, 2H), 3.23 (m, 2H), 3.02 (s, 6H).

### Synthesis of Example 29: 6-(4-(5-((7-(Cyclobutylamino)-4-oxo-3,4-dihydrophthalazin-1-yl)methyl)-2-fluorobenzoyl)piperazin-1-yl)nicotinonitrile

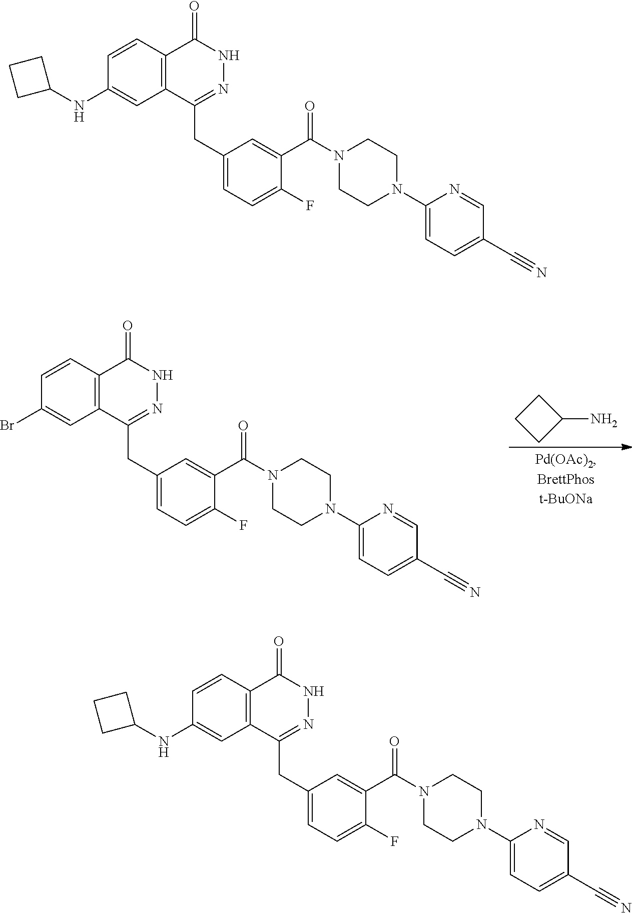 [SMILES: N#Cc1ccc(N2CCN(C(=O)c3cc(Cc4n[nH]c(=O)c5ccc(NC6CCC6)cc45)ccc3F)CC2)nc1 ; N#Cc1ccc(N2CCN(C(=O)c3cc(Cc4n[nH]c(=O)c5ccc(Br)cc45)ccc3F)CC2)nc1 ; NC1CCC1 ; N#Cc1ccc(N2CCN(C(=O)c3cc(Cc4n[nH]c(=O)c5ccc(NC6CCC6)cc45)ccc3F)CC2)nc1]

### Step 1

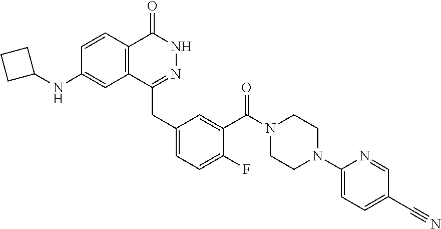 [SMILES: N#Cc1ccc(N2CCN(C(=O)c3cc(Cc4n[nH]c(=O)c5ccc(NC6CCC6)cc45)ccc3F)CC2)nc1]

### 6-(4-(5-((7-(Cyclobutylamino)-4-oxo-3,4-dihydrophthalazin-1-yl)methyl)-2-fluorobenzoyl)piperazin-1-yl)nicotinonitrile

A mixture of 6-(4-(5-((7-bromo-4-oxo-3,4-dihydrophthalazin-1-yl)methyl)-2-fluorobenzoyl)piperazin-1-yl)nicotinonitrile (200 mg, 0.37 mmol), cyclobutanamine (130 mg, 1.83 mmol), Pd(OAc)₂ (15 mg, 0.07 mmol), Brettphos (39 mg, 0.07 mmol) and NaOtBu (70 mg, 0.73 mmol) in DMA (2 mL) was stirred at 130° C. under Ar (g) for 1 hour. The reaction was cooled to rt and purified by pre-HPLC to give 6-(4-(5-((7-(cyclobutylamino)-4-oxo-3,4-dihydrophthalazin-1-yl)methyl)-2-fluorobenzoyl)piperazin-1-yl)nicotinonitrile as a white solid (19.6 mg, 10% yield). LCMS ESI m/z 538 [M+H]⁺. ¹H NMR (400 MHz, CDCl₃) δ 9.62 (s, 1H), 8.42 (d, J=2.0 Hz, 1H), 8.18 (d, J=8.7 Hz, 1H), 7.66 (dd, J=9.0, 2.3 Hz, 1H), 7.37-7.34 (m, 1H), 7.33-7.29 (m, 1H), 7.06 (t, J=8.8 Hz, 1H), 6.86 (dd, J=8.7, 2.2 Hz, 1H), 6.62 (d, J=9.0 Hz, 1H), 6.48 (d, J=2.2 Hz, 1H), 4.62 (d, J=6.2 Hz, 1H), 4.18 (s, 2H), 3.94-3.86 (m, 3H), 3.81-3.75 (m, 2H), 3.68 (m, 2H), 3.43 (m, 2H), 2.45-2.37 (m, 2H), 1.86 (m, 4H).

### Synthesis of Example 30: 6-(4-(2-fluoro-5-((4-oxo-7-(trifluoromethoxy)-3,4-dihydrophthalazin-1-yl)methyl)benzoyl)piperazin-1-yl)nicotinonitrile

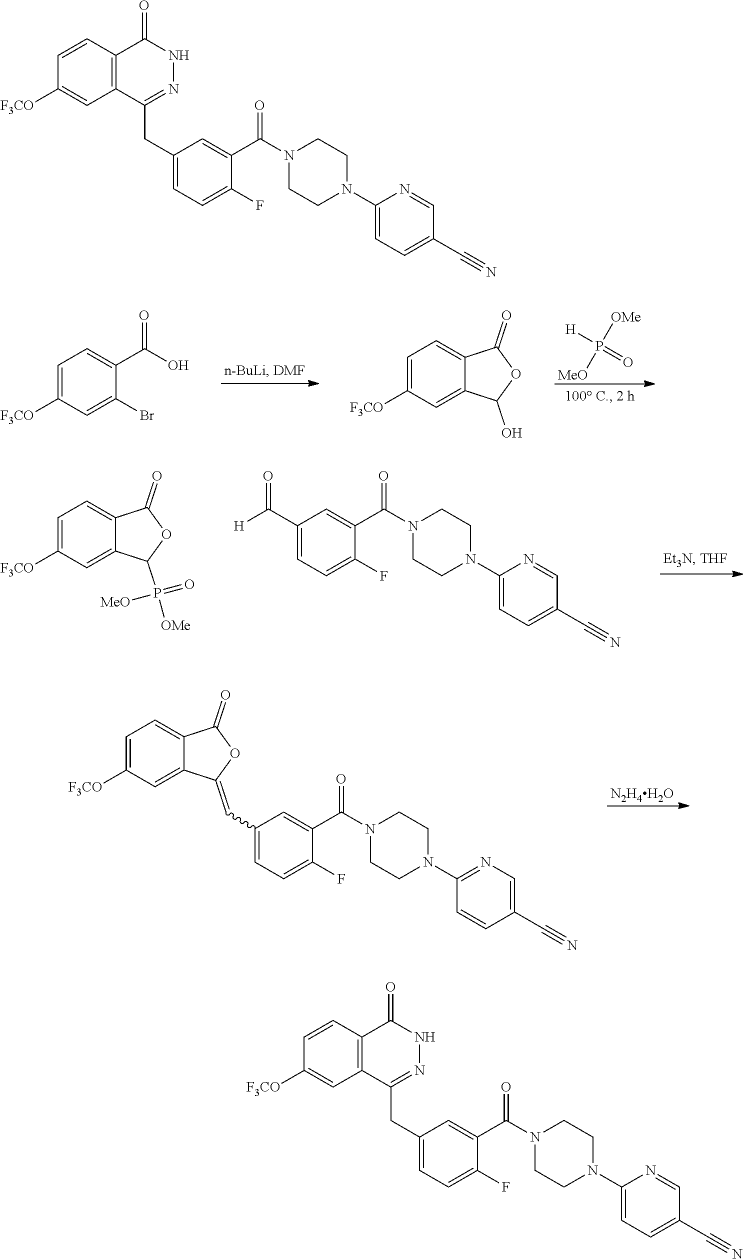

### Step 1

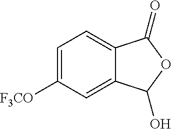 [SMILES: O=C1OC(O)c2cc(OC(F)(F)F)ccc21]

### 3-Hydroxy-5-(trifluoromethoxy)isobenzofuran-1(3H)-one

A solution of 2-bromo-4-(trifluoromethoxy)benzoic acid (200 mg, 0.70 mmol) in THF (6 mL) was cooled to −78° C., then n-BuLi (2.5M in heptane, 0.56 mL, 1.4 mmol) was added dropwise under Ar (g). The reaction was stirred at −78° C. for 15 min, then DMF (112 mg, 1.54 mmol) was added. The reaction was kept at −78° C. for 20 min, then quenched with sat. NH₄Cl solution (50 mL). The resulting mixture was added aq HCl (1N) until the solution reached pH 5˜6, then extracted with EtOAc (30×3 mL). The combined organic layer was washed with bine, dried over Na₂SO₄, filtered and concentrated in vacuo. The residue was purified by silica gel chromatography (EtOAc/petroleum ether=30 to 50%) to give 3-hydroxy-5-(trifluoromethoxy)isobenzofuran-1(3H)-one as a white solid (75 mg, 45% yield). LCMS ESI m/z 235 (M+H)⁺.

### Step 2

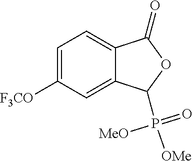 [SMILES: COP(=O)(OC)C1OC(=O)c2ccc(OC(F)(F)F)cc21]

### Dimethyl (3-oxo-6-(trifluoromethoxy)-1,3-dihydroisobenzofuran-1-yl)phosphonate

A solution of 3-hydroxy-5-(trifluoromethoxy)isobenzofuran-1(3H)-one (70 mg, 0.30 mmol) in dimethylphosphite (5 mL) was heated to 100° C. and stirred for 2 hours. The mixture was cooled to rt, diluted with H₂O (10 mL) and extracted with EtOAc (10 mL×3). The combined organic layer was washed with brine, dried over anhydrous Na₂SO₄, filtered and concentrated in vacuo to give dimethyl (6-(difluoromethoxy)-3-oxo-1,3-dihydroisobenzofuran-1-yl)phosphonate as a light yellow oil (90 mg, 83% yield). LCMS ESI m/z 327 (M+H)⁺.

### Step 3

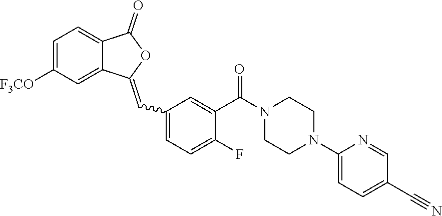 [SMILES: N#Cc1ccc(N2CCN(C(=O)c3cc(C=C4OC(=O)c5ccc(OC(F)(F)F)cc54)ccc3F)CC2)nc1]

### 6-(4-(2-Fluoro-5-((3-oxo-6-(trifluoromethoxy)isobenzofuran-1(3H)-ylidene)methyl)benzoyl)piperazin-1-yl)nicotinonitrile

A solution of dimethyl (3-oxo-6-(trifluoromethoxy)-1,3-dihydroisobenzofuran-1-yl)phosphonate (90 mg, 0.28 mmol), 6-(4-(2-fluoro-5-formylbenzoyl)piperazin-1-yl)nicotinonitrile (112 mg, 0.33 mmol) and Et₃N (84 mg, 0.83 mmol) in THF (3 mL) was stirred at rt under Ar (g) for 12 hours. The mixture was diluted with water (10 mL) and extracted with EtOAc (10 mL×3). The combined organic layer was washed with brine, dried over Na₂SO₄, filtered and concentrated in vacuo to give 6-(4-(2-fluoro-5-((3-oxo-6-(trifluoromethoxy)isobenzofuran-1(3H)-ylidene)methyl)benzoyl)piperazin-1-yl)nicotinonitrile as a yellow solid (140 mg, 94% yield). LCMS ESI m/z 539 (M+H)⁺.

### Step 4

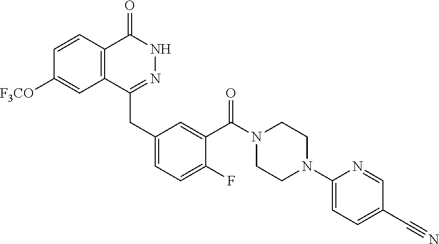 [SMILES: N#Cc1ccc(N2CCN(C(=O)c3cc(Cc4n[nH]c(=O)c5ccc(OC(F)(F)F)cc45)ccc3F)CC2)nc1]

### 6-(4-(2-fluoro-5-((4-oxo-7-(trifluoromethoxy)-3,4-dihydrophthalazin-1-yl)methyl)benzoyl)piperazin-1-yl)nicotinonitrile

A mixture of 6-(4-(2-fluoro-54(3-oxo-6-(trifluoromethoxy)isobenzofuran-1(3H)-ylidene)methyl)benzoyl)piperazin-1-yl)nicotinonitrile (70 mg, 0.13 mmol) and N₂H₄·H₂O (77 mg, 1.3 mmol) in THF (2 mL) was stirred at 70° C. for 3 hours. The solids were filtered and washed with water (5 mL), dried under vacuum to give 6-(4-(2-fluoro-5-((4-oxo-7-(trifluoromethoxy)-3,4-dihydrophthalazin-1-yl)methyl)benzoyl)piperazin-1-yl)nicotinonitrile as a white solid (26 mg, 36% yield). LCMS ESI m/z 553 (M+H)⁺. ¹H NMR (400 MHz, CDCl₃) δ 10.02 (s, 1H), 8.52 (d, J=8.8 Hz, 1H), 8.42 (d, J=2.1 Hz, 1H), 7.66 (dd, J=9.0, 2.3 Hz, 1H), 7.57 (d, J=8.8 Hz, 1H), 7.49 (s, 1H), 7.39-7.32 (m, 2H), 7.09 (t, J=9.0 Hz, 1H), 6.62 (d, J=9.0 Hz, 1H), 4.26 (s, 2H), 3.90 (m, 2H), 3.78 (m, 2H), 3.69 (t, J=5.1 Hz, 2H), 3.42 (m, 2H).

### Synthesis of Example 31: 6-(4-(2-Fluoro-5-((4-oxo-7-(2-azaspiro[3.3]heptan-2-yl)-3,4-dihydrophthalazin-1-yl)methyl)benzoyl)piperazin-1-yl)nicotinonitrile

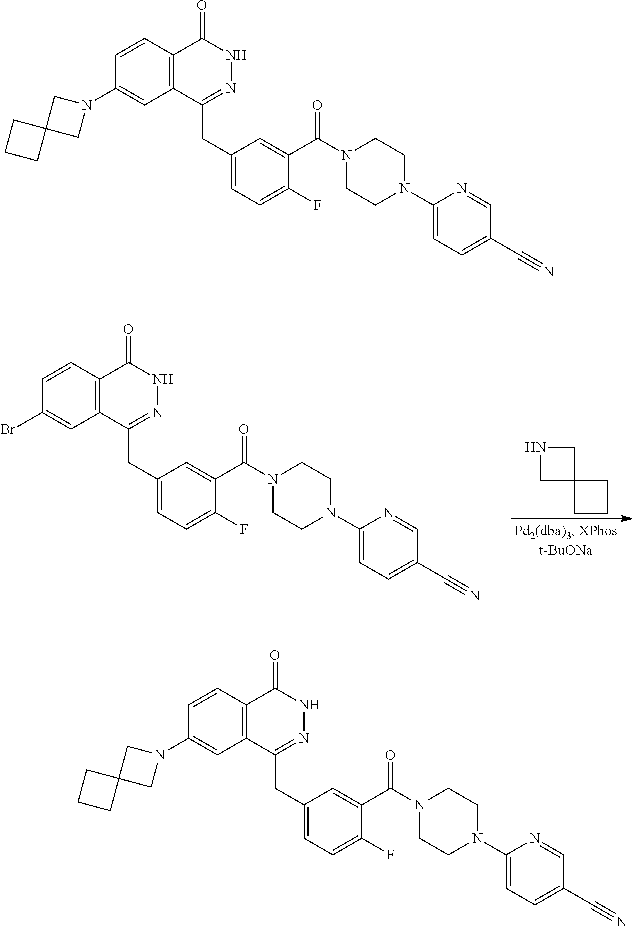 [SMILES: N#Cc1ccc(N2CCN(C(=O)c3cc(Cc4n[nH]c(=O)c5ccc(N6CC7(CCC7)C6)cc45)ccc3F)CC2)nc1 ; N#Cc1ccc(N2CCN(C(=O)c3cc(Cc4n[nH]c(=O)c5ccc(Br)cc45)ccc3F)CC2)nc1 ; C1CC2(C1)CNC2 ; N#Cc1ccc(N2CCN(C(=O)c3cc(Cc4n[nH]c(=O)c5ccc(N6CC7(CCC7)C6)cc45)ccc3F)CC2)nc1]

### Step 1

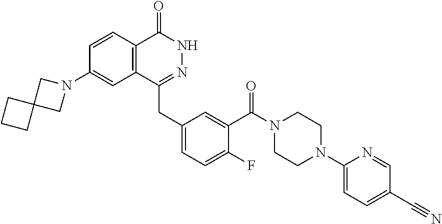 [SMILES: N#Cc1ccc(N2CCN(C(=O)c3cc(Cc4n[nH]c(=O)c5ccc(N6CC7(CCC7)C6)cc45)ccc3F)CC2)nc1]

### 6-(4-(2-Fluoro-5-((4-oxo-7-(2-azaspiro[3.3]heptan-2-yl)-3,4-dihydrophthalazin-1-yl)methyl)benzoyl)piperazin-1-yl)nicotinonitrile

6-[4-[5-[(7-Bromo-4-oxo-3H-phthalazin-1-yl)methyl]-2-fluoro-benzoyl]piperazin-1-yl]pyridine-3-carbonitrile (50.0 mg, 91.3 μmol), 2-azaspiro[3.3]heptane (44.4 mg, 457 μmol), Pd₂(dba)₃ (5.3 mg, 9.1 μmol), t-BuONa (61.5 mg, 639 μmol), XPhos (8.7 mg, 18.3 μmol) and DMA (3 mL) were added to a 10 mL scaled tube. The resultant mixture was stirred at 120° C. for 0.5 hr under N₂ (g). The mixture was purified by prep-HPLC to afford 6-[4-[5-[[7-(2-azaspiro[3.3]heptan-2-yl)-4-oxo-3H-phthalazin-1-yl]methyl]-2-fluoro-benzoyl]piperazin-1-yl]pyridine-3-carbonitrile (8.05 mg, 16% yield) as a white solid. LCMS ESI m/z 563.8 [M+H]⁺. ¹H NMR (400 MHz, DMSO-d₆) δ 12.14 (s, 1H), 8.51 (d, J=2.1 Hz, 1H), 7.99 (d, J=8.7 Hz, 1H), 7.90 (dd, J=9.1, 2.4 Hz, 1H), 7.42 (dd, J=8.8, 5.8 Hz, 2H), 7.25 (t, J=8.9 Hz, 1H), 6.92 (d, J=9.1 Hz, 1H), 6.81 (dd, J=8.7, 2.1 Hz, 1H), 6.52 (d, J=2.1 Hz, 1H), 4.22 (s, 2H), 3.90 (s, 4H), 3.75 (m, 4H), 3.62 (m, 2H), 3.37 (m, 2H), 2.17 (t, J=7.5 Hz, 4H), 1.85-1.76 (m, 2H).

### Synthesis of Example 32: (E)-6-(4-(2-fluoro-5-((4-oxo-7-(4,4,4-trifluorobut-2-en-2-yl)-3,4-dihydrophthalazin-1-yl)methyl)benzoyl)piperazin-1-yl)nicotinonitrile

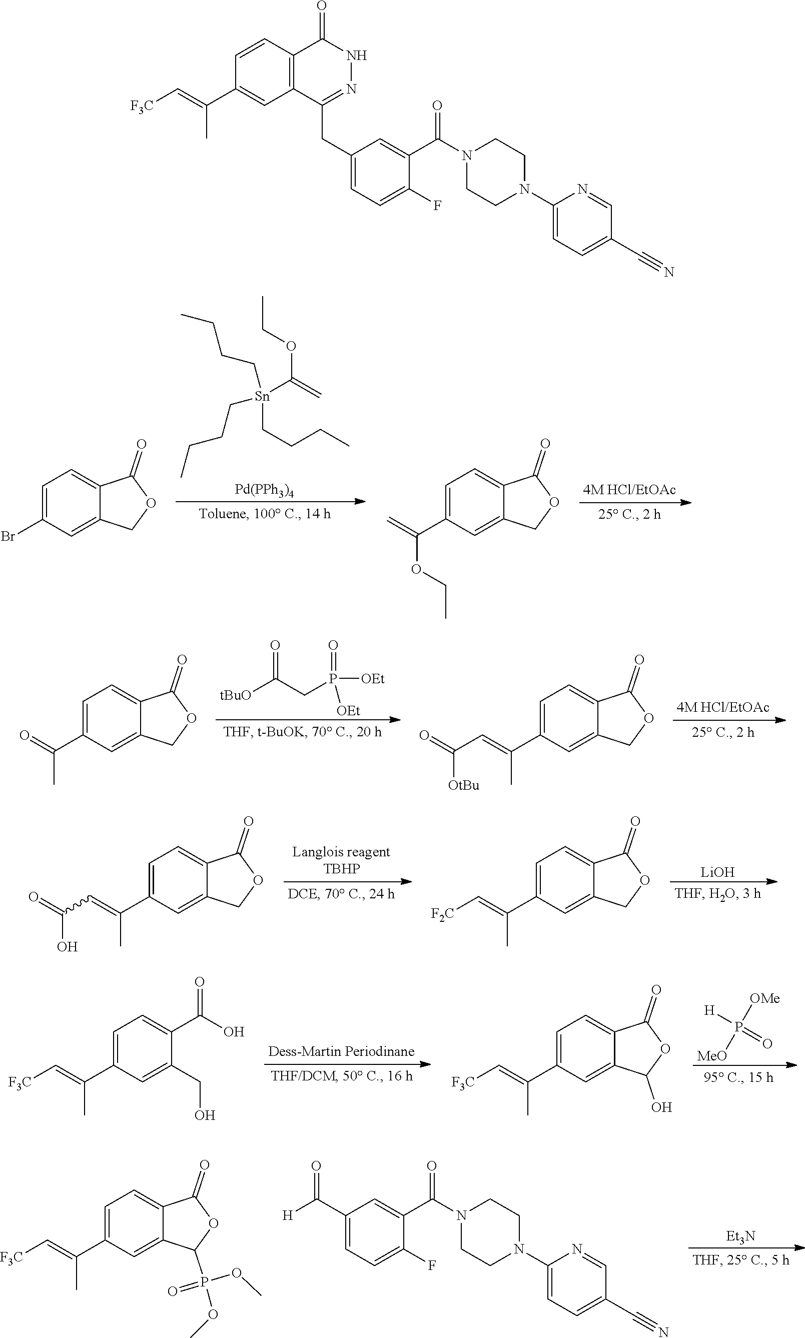

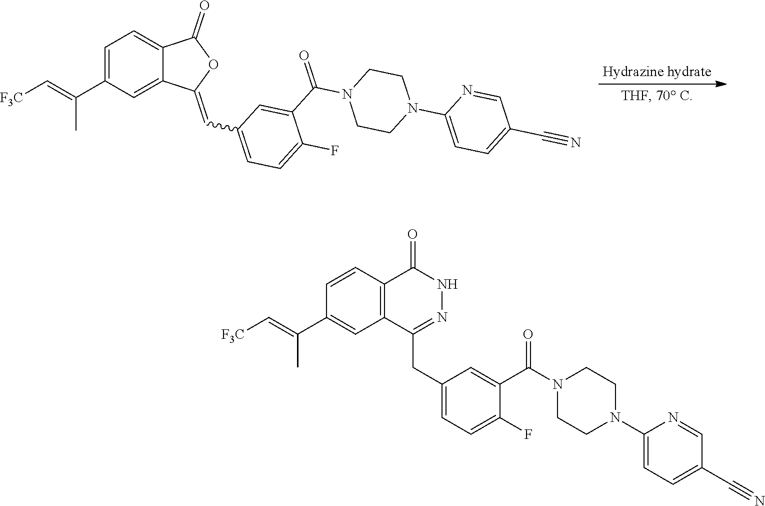 [SMILES: C/C(=C\C(F)(F)F)c1ccc2c(c1)C(=Cc1ccc(F)c(C(=O)N3CCN(c4ccc(C#N)cn4)CC3)c1)OC2=O ; C/C(=C\C(F)(F)F)c1ccc2c(=O)[nH]nc(Cc3ccc(F)c(C(=O)N4CCN(c5ccc(C#N)cn5)CC4)c3)c2c1]

### Step 1

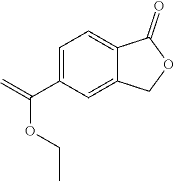

### 5-(1-Ethoxyvinyl)isobenzofuran-1(3H)-one

To a solution of 5-bromo-3H-isobenzofuran-1-one (3.00 g, 164 mmol) in toluene (101 mL) was added tributyl(1-ethoxyvinyl)stannane (89.0 g, 246 mmol, 83.3 mL), Pd(PPh₃)₄ (9.49 g, 8.21 mmol) at 25° C. under N₂. The reaction mixture was stirred at 100° C. for 14 hrs. Water and EtOAc were added to the mixture. The organic layer was separated, washed with NaCl (aq), dried over Na₂SO₄ and concentrated to give a residue. The residue was purified by silica gel chromatography (petroleum ether/EtOAc=20/1 to 0/1) to give 5-(1-ethoxyvinyl)-3H-isobenzofuran-1-one (30 g, 89% yield) as a yellow solid. ¹H NMR (400 MHz, DMSO-D6) δ 7.89 (s, 1H), 7.82 (s, 2H), 5.41 (s, 2H), 4.98 (d, J=3.0 Hz, 1H), 4.49 (d, J=3.0 Hz, 1H), 3.93 (t, J=7.0 Hz, 2H), 1.37 (t, J=7.0 Hz, 3H).

### Step 2

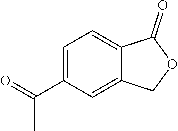

### 5-Acetylisobenzofuran-1(3H)-one

A solution of 5-(1-ethoxyvinyl)-3H-isobenzofuran-1-one (30 g, 146.90 mmol) in 4M HCl/EtOAc (180 mL) was stirred at 25° C. for 2 hrs. The reaction mixture was concentrated to give 5-acetyl-3H-isobenzofuran-1-one (25 g, 96% yield) as a white solid. ¹H NMR (400 MHz, DMSO-d₆) δ 8.23 (s, 1H), 8.12 (d, J=8.0 Hz, 1H), 7.97 (d, J=8.0 Hz, 1H), 5.49 (s, 2H), 2.67 (s, 3H).

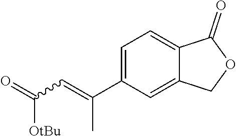 [SMILES: CC(=CC(=O)OC(C)(C)C)c1ccc2c(c1)COC2=O]

### (Z)- and (E)-tert-Butyl 3-(1-oxo-1,3-dihydroisobenzofuran-5-yl)but-2-enoate

To a solution of 5-acetyl-3H-isobenzofuran-1-one (1.80 g, 10.2 mmol) in THF (20 mL) was added tert-butyl 2-diethoxyphosphorylacetate (3.35 g, 13.3 mmol, 3.12 mL) and t-BuOK (1.49 g, 13.3 mmol), The reaction mixture was stirred at 70° C. for 20 hrs. The reaction mixture was concentrated under vacuum. The residue was poured into water, extracted with EtOAc. The combined organic extracts were dried by Na₂SO₄, filtered and evaporated under reduced pressure to give viscous oils, which were purified by silica gel chromatography (petroleum ether/EtOAc=10/1 to 2/1) to give the mixture of (E)-tert-butyl 3-(1-oxo-1,3-dihydroisobenzofuran-5-yl)but-2-enoate and (Z)-tert-butyl 3-(1-oxo-1,3-dihydroisobenzofuran-5-yl)but-2-enoate (1.2 g, 43% yield) as a yellow solid. ¹H NMR (400 MHz, DMSO-d6) δ 7.85 (d, J=8.2 Hz, 2H), 7.73 (d, J=8.7 Hz, 1H), 6.13 (d, J=1.2 Hz, 1H), 5.41 (d, J=6.7 Hz, 2H), 2.51 (d, J=1.2 Hz, 3H), 1.48 (s, 9H).

### Step 4

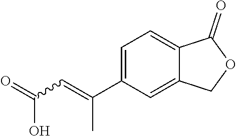 [SMILES: CC(=CC(=O)O)c1ccc2c(c1)COC2=O]

### (Z)- and (E)-3-(1-Oxo-1,3-dihydroisobenzofuran-5-yl)but-2-enoic acid

The mixture product from above (8.00 g, 29.2 mmol) was added to 4M HCl/EtOAc (4 M, 100 mL). The reaction mixture was stirred at 25° C. for 3 hrs. The reaction mixture was concentrated to give a mixture of (E)-3-(1-oxo-1,3-dihydroisobenzofuran-5-yl)but-2-enoic acid and (Z)-3-(1-oxo-1,3-dihydroisobenzofuran-5-yl)but-2-enoic acid (5.78 g, 91% yield) was a yellow solid. LCMS ESI m/z: 219.1 [M+H]⁺.

### Step 5

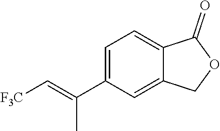 [SMILES: C/C(=C\C(F)(F)F)c1ccc2c(c1)COC2=O]

### (E)-5-(4,4,4-trifluorobut-2-en-2-yl)isobenzofuran-1(3H)-one

To a solution of mixture (E)-3-(1-oxo-1,3-dihydroisobenzofuran-5-yl)but-2-enoic and (Z)-3-(1-oxo-1,3-dihydroisobenzofuran-5-yl)but-2-enoic acid (100 mg, 458 μmol), CuCl (45.4 mg, 458 μmol), Ag₂CO₃ (75.8 mg, 275 μmol), and CF₃SO₂Na (215 mg, 1.37 mmol) in DCE (2 mL) at 0° C. was slowly added TBHP (295 mg, 2.29 mmol, 70% purity) with stirring. The reaction was allowed to warm to 70° C. and then stirred for 24 h under N₂. Water and EtOAc were added to the mixture, which became two phases. The organic layer was separated, washed with NaCl (aq), dried over Na₂SO₄ and concentrated to give a residue. The residue was purified by prepared-TLC (petroleum ether/EtOAc=2/1) to give (E)-5-(4,4,4-trifluorobut-2-en-2-yl)isobenzofuran-1(3H)-one (25 mg, 23% yield) as a yellow solid. ¹H NMR (400 MHz, DMSO-d₆) δ 7.92-7.85 (m, 2H), 7.78 (d, J=8.2 Hz, 1H), 6.34 (dd, J=8.6, 1.3 Hz, 1H), 5.44 (s, 2H), 2.32 (dd, J=2.1, 1.4 Hz, 3H).

### Step 6

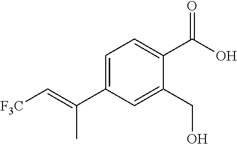 [SMILES: C/C(=C\C(F)(F)F)c1ccc(C(=O)O)c(CO)c1]

### (E)-2-(Hydroxymethyl)-4-(4,4,4-trifluorobut-2-en-2-yl)benzoic acid

To a solution of (E)-5-(4,4,4-trifluorobut-2-en-2-yl)isobenzofuran-1(3H)-one (630 mg, 2.60 mmol) in THF (3 mL) was added LiOH (186.90 mg, 7.80 mmol) at 25° C. The reaction mixture was stirred at 25° C. for 3 hr. The mixture was adjusted to pH 6 with 1M HCl (aq). EtOAc was added to the mixture, which became two phases. The organic layer was separated, washed with NaCl (aq), dried over Na₂SO₄ and concentrated to give (E)-2-(hydroxymethyl)-4-(4,4,4-trifluorobut-2-en-2-yl)benzoic acid (400 mg, 59% yield) as a white solid. LCMS ESI m/z: 258.9 [M+H]⁺.

### Step 7

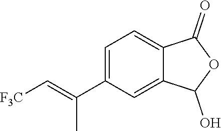 [SMILES: C/C(=C\C(F)(F)F)c1ccc2c(c1)C(O)OC2=O]

### (E)-3-Hydroxy-5-(4,4,4-trifluorobut-2-en-2-yl)isobenzofuran-1(3H)-one

To a solution of (E)-2-(hydroxymethyl)-4-(4,4,4-trifluorobut-2-en-2-yl)benzoic acid (800 mg, 3.07 mmol) in DCM (6 mL) was added Dess-Martin Periodinane (1.51 g, 3.38 mmol, 95% purity) at 25° C. under N₂. The reaction mixture was stirred at 50° C. for 16 hrS. DCM was added to the mixture, after stirring the mixture for 30 min, the solids were collected, washed with DCM and filtrate dried under reduced pressure to give the crude product. The crude was purified by silica gel chromatography (petroleum ether/EtOAc=20/1 to 0/1) to give (E)-3-hydroxy-5-(4,4,4-trifluorobut-2-en-2-yl)isobenzofuran-1(3H)-one (450 mg, 57% yield) as a yellow oil. LCMS ESI m/z: 256.8 [M+H]⁺.

### Step 8, 9 and 10

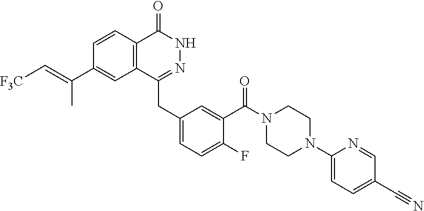 [SMILES: C/C(=C\C(F)(F)F)c1ccc2c(=O)[nH]nc(Cc3ccc(F)c(C(=O)N4CCN(c5ccc(C#N)cn5)CC4)c3)c2c1]

### (E)-6-(4-(2-Fluoro-5-((4-oxo-7-(4,4,4-trifluorobut-2-en-2-yl)-3,4-dihydrophthalazin-1-yl)methyl)benzoyl)piperazin-1-yl)nicotinonitrile

Following the three-step procedure in Example 1, but starting with (E)-3-hydroxy-5-(4,4,4-trifluorobut-2-en-2-yl)isobenzofuran-1(3H)-one gave the title compound as a white solid. LCMS ESI m/z: 576.7 [M+H]⁺. ¹H NMR (400 MHz, DMSO-d6) δ12.64 (s, 1H), 8.51 (d, J=2.1 Hz, 1H), 8.26 (d, J=8.3 Hz, 1H), 8.09 (s, 1H), 8.03 (d, J=8.4 Hz, 1H), 7.90 (dd, J=9.1, 2.3 Hz, 1H), 7.46 (t, J=6.9 Hz, 2H), 7.26 (t, J=8.9 Hz, 1H), 6.92 (d, J=9.1 Hz, 1H), 6.41 (dd, J=16.8, 8.1 Hz, 1H), 4.42 (s, 2H), 3.74 (m, 4H), 3.60 (m, 2H), 3.30-3.26 (m, 2H), 2.32 (s, 3H).

### Synthesis of Example 33: 6-(4-(2-Fluoro-5-((4-oxo-7-((3,3,3-trifluoropropyl)amino)-3,4-dihydrophthalazin-1-yl)methyl)benzoyl)piperazin-1-yl)nicotinonitrile

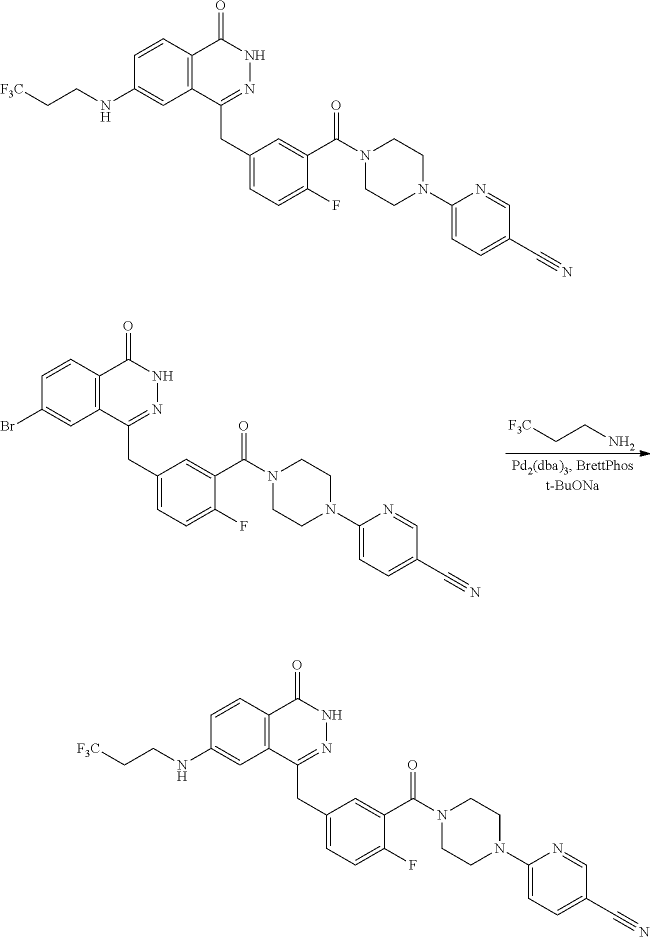 [SMILES: N#Cc1ccc(N2CCN(C(=O)c3cc(Cc4n[nH]c(=O)c5ccc(NCCC(F)(F)F)cc45)ccc3F)CC2)nc1 ; N#Cc1ccc(N2CCN(C(=O)c3cc(Cc4n[nH]c(=O)c5ccc(Br)cc45)ccc3F)CC2)nc1 ; NCCC(F)(F)F ; N#Cc1ccc(N2CCN(C(=O)c3cc(Cc4n[nH]c(=O)c5ccc(NCCC(F)(F)F)cc45)ccc3F)CC2)nc1]

### Step 1

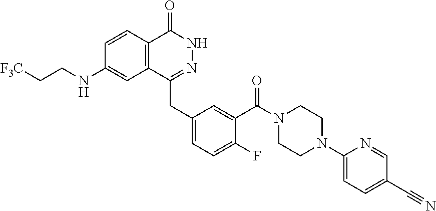 [SMILES: N#Cc1ccc(N2CCN(C(=O)c3cc(Cc4n[nH]c(=O)c5ccc(NCCC(F)(F)F)cc45)ccc3F)CC2)nc1]

### 6-(4-(2-Fluoro-5-((4-oxo-7-((3,3,3-trifluoropropyl)amino)-3,4-dihydrophthalazin-1-yl)methyl)benzoyl)piperazin-1-yl)nicotinonitrile

A mixture of 6-(4-(5-((7-bromo-4-oxo-3,4-dihydrophthalazin-1-yl)methyl)-2-fluorobenzoyl)piperazin-1-yl)nicotinonitrile (200 mg, 0.37 mmol), 3,3-trifluoropropan-1-amine (124 mg, 1.10 mmol), Pd₂(dba)₃ (33 mg, 0.04 mmol), BrettPhos (39.0 mg, 0.07 mmol) and t-BuONa (211 mg, 2.19 mmol) in DMA (2 mL) was heated at 160° C. by microwave for 0.5 hour. The reaction was cooled to rt and purified by prep-HPLC to give 6-(4-(2-fluoro-5-((4-oxo-7-((3,3,3-trifluoropropyl)amino)-3,4-dihydrophthalazin-1-yl)methyl)benzoyl)piperazin-1-yl)nicotinonitrile as a white solid (51 mg, 24% yield). LCMS ESI m/z: 580 [M+H]⁺. ¹H NMR (400 MHz, CDCl₃) δ 9.81 (s, 1H), 8.42 (d, J=1.9 Hz, 1H), 8.23 (d, J=8.7 Hz, 1H), 7.66 (dd, J=9.0, 2.3 Hz, 1H), 7.38-7.35 (m, 1H), 7.34-7.30 (m, 1H), 7.06 (t, J=8.8 Hz, 1H), 6.93 (dd, J=8.8, 2.3 Hz, 1H), 6.62 (d, J=9.0 Hz, 1H), 6.58 (d, J=2.2 Hz, 1H), 4.65 (t, J=6.0 Hz, 1H), 4.20 (s, 2H), 3.89 (m, 2H), 3.78 (m, 2H), 3.70 (t, J=5.1 Hz, 2H), 3.52-3.41 (m, 4H), 2.45-2.33 (m, 2H).

### Synthesis of Example 34: 6-(4-(2-Fluoro-5-((4-oxo-7-(3-(trifluoromethyl)azetidin-1-yl)-3,4-dihydrophthalazin-1-yl)methyl)benzoyl)piperazin-1-yl)nicotinonitrile

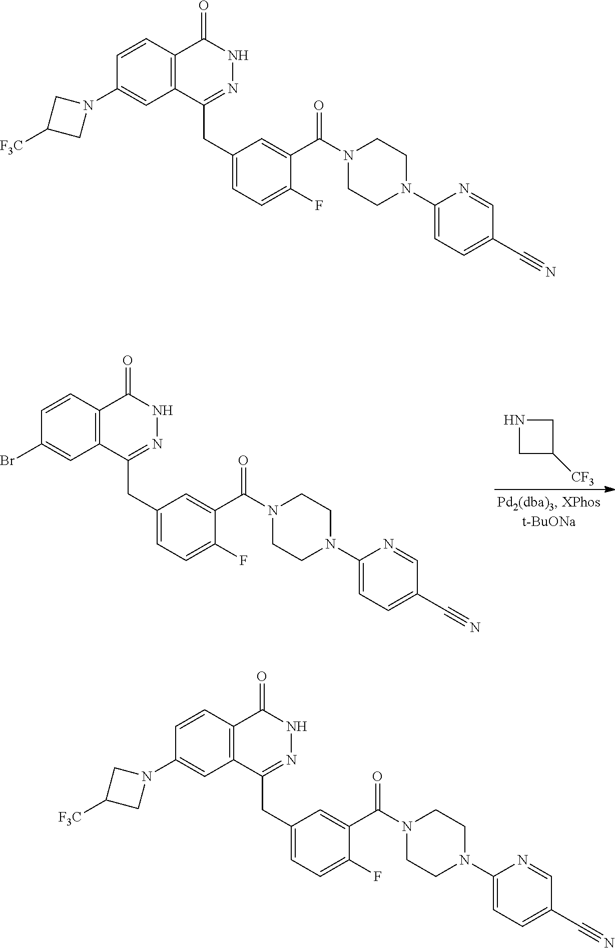 [SMILES: N#Cc1ccc(N2CCN(C(=O)c3cc(Cc4n[nH]c(=O)c5ccc(N6CC(C(F)(F)F)C6)cc45)ccc3F)CC2)nc1 ; N#Cc1ccc(N2CCN(C(=O)c3cc(Cc4n[nH]c(=O)c5ccc(Br)cc45)ccc3F)CC2)nc1 ; FC(F)(F)C1CNC1 ; N#Cc1ccc(N2CCN(C(=O)c3cc(Cc4n[nH]c(=O)c5ccc(N6CC(C(F)(F)F)C6)cc45)ccc3F)CC2)nc1]

### Step 1

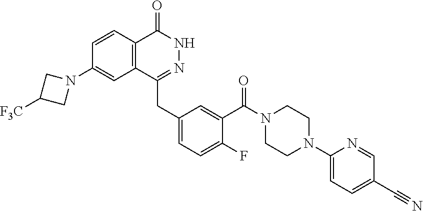 [SMILES: N#Cc1ccc(N2CCN(C(=O)c3cc(Cc4n[nH]c(=O)c5ccc(N6CC(C(F)(F)F)C6)cc45)ccc3F)CC2)nc1]

### 6-(4-(2-Fluoro-5-((4-oxo-7-(3-(trifluoromethyl)azetidin-1-yl)-3,4-dihydrophthalazin-1-yl)methyl)benzoyl)piperazin-1-yl)nicotinonitrile

To a solution of 6-[4-[5-[(7-bromo-4-oxo-3H-phthalazin-1-yl)methyl]-2-fluoro-benzoyl]piperazin-1-yl]pyridine-3-carbonitrile (50.0 mg, 91.3 μmol) in DMA (2 mL) was added 3-(trifluoromethyl)azetidine hydrochloride (73.8 mg, 456 μmol), t-BuONa (61.5 mg, 639 μmol), Pd₂(dba)₃ (8.4 mg, 9.1 μmol) and XPhos (8.7 mg, 18.3 μmol). The reaction mixture was stirred at 110° C. under microwave for 0.5 hour. The mixture was purified by Prep-HPLC to give 6-[4-[2-fluoro-5-[[4-oxo-7-[3-(trifluoromethyl)azetidin-1-yl]-3H-phthalazin-1-yl]methyl]benzoyl]piperazin-1-yl]pyridine-3-carbonitrile (15.6 mg, 29% yield) as a white solid. LCMS ESI m/z: 591.8 [M+H]⁺. ¹H NMR (400 MHz, DMSO-d₆) δ 12.22 (s, 1H), 8.51 (d, J=2.1 Hz, 1H), 8.04 (d, J=8.7 Hz, 1H), 7.90 (dd, J=9.1, 2.4 Hz, 1H), 7.50-7.41 (m, 2H), 7.25 (t, J=9.0 Hz, 1H), 6.92 (dd, J=8.8, 2.2 Hz, 2H), 6.68 (d, J=2.1 Hz, 1H), 4.25 (s, 2H), 4.18 (t, J=8.7 Hz, 2H), 4.02 (dd, J=8.8, 5.1 Hz, 2H), 3.75 (m, 5H), 3.62 (m, 2H), 3.32-3.29 (m, 2H).

### Synthesis of Example 35: 6-(4-(5-((7-(6,6-Difluoro-2-azaspiro[33]heptan-2-yl)-4-oxo-3,4-dihydrophthalazin-1-yl)methyl)-2-fluorobenzoyl)piperazin-1-yl)nicotinonitrile

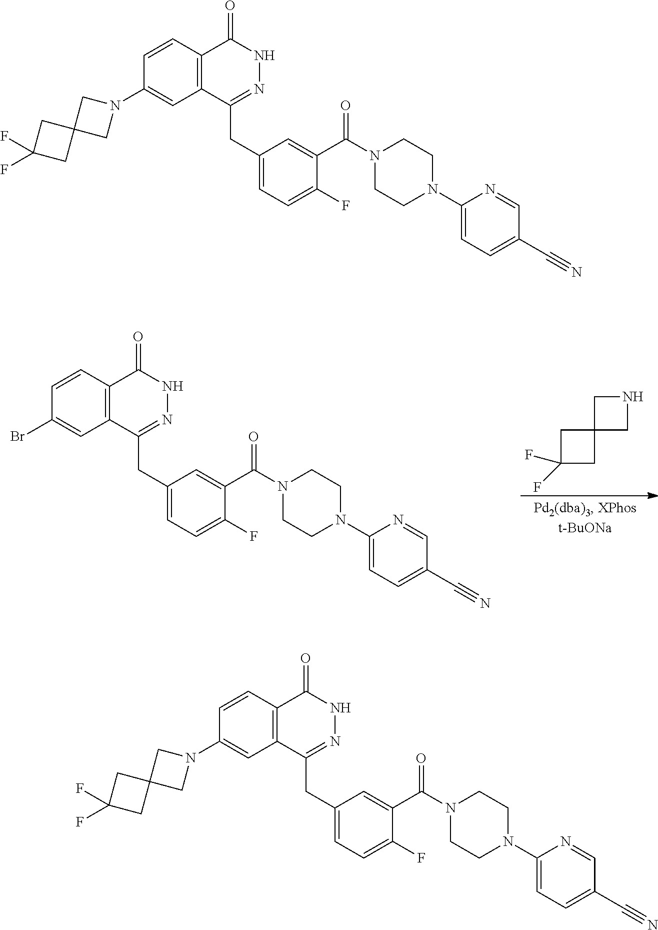 [SMILES: N#Cc1ccc(N2CCN(C(=O)c3cc(Cc4n[nH]c(=O)c5ccc(N6CC7(C6)CC(F)(F)C7)cc45)ccc3F)CC2)nc1 ; N#Cc1ccc(N2CCN(C(=O)c3cc(Cc4n[nH]c(=O)c5ccc(Br)cc45)ccc3F)CC2)nc1 ; FC1(F)CC2(CNC2)C1 ; N#Cc1ccc(N2CCN(C(=O)c3cc(Cc4n[nH]c(=O)c5ccc(N6CC7(C6)CC(F)(F)C7)cc45)ccc3F)CC2)nc1]

### Step 1

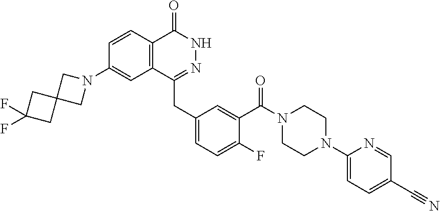 [SMILES: N#Cc1ccc(N2CCN(C(=O)c3cc(Cc4n[nH]c(=O)c5ccc(N6CC7(C6)CC(F)(F)C7)cc45)ccc3F)CC2)nc1]

### 6-(4-(5-((7-(6,6-Difluoro-2-azaspiro[3.3]heptan-2-yl)-4-oxo-3,4-dihydrophthalazin-1-yl)methyl)-2-fluorobenzoyl)piperazin-1-yl)nicotinonitrile

A mixture of 6-(4-(5-((7-bromo-4-oxo-3,4-dihydrophthalazin-1-yl)methyl)-2-fluorobenzoyl)piperazin-1-yl)nicotinonitrile (200 mg, 0.37 mmol), 6,6-difluoro-2-azaspiro[3.3]heptane (200 mg, 1.48 mmol), Pd₂(dba), (65.0 mg, 0.07 mmol), XPhos (33.0 mg, 0.07 mmol) and t-BuONa (107 mg, 1.11 mmol) in DMA (10 mL) was stirred at 130° C. for 16 hours. The reaction was cooled to rt, then the solids were filtered off. The filtrate was concentrated in vacuo and the residue was purified by prep-HPLC to give 6-(4-(5-((7-(6,6-difluoro-2-azaspiro[3.3]heptan-2-yl)-4-oxo-3,4-dihydrophthalazin-1-yl)methyl)-2-fluorobenzoyl)piperazin-1-yl)nicotinonitrile as a white solid (28 mg, 13% yield). LCMS ESI m/z: 600 (M+H)⁺. ¹H NMR (400 MHz, DMSO-d₆) δ 12.18 (s, 1H), 8.51 (d, J=2.1 Hz, 1H), 8.01 (d, J=8.7 Hz, 1H), 7.90 (dd, J=9.1, 2.4 Hz, 1H), 7.47-7.38 (m, 2H), 7.25 (t, J=8.9 Hz, 1H), 6.93 (d, J=9.0 Hz, 1H), 6.85 (dd, J=8.7, 2.1 Hz, 1H), 6.59 (d, J=2.1 Hz, 1H), 4.23 (s, 2H), 4.05 (s, 4H), 3.76 (m, 4H), 3.62 (m, 2H), 3.36 (m, 2H), 2.87 (t, J=12.4 Hz, 4H).

### Synthesis of Example 36: 6-(4-(5-((7-(1,1-Difluoroethyl)-4-oxo-3,4-dihydrophthalazin-1-yl)methyl)-2-fluorobenzoyl)piperazin-1-yl)nicotinonitrile

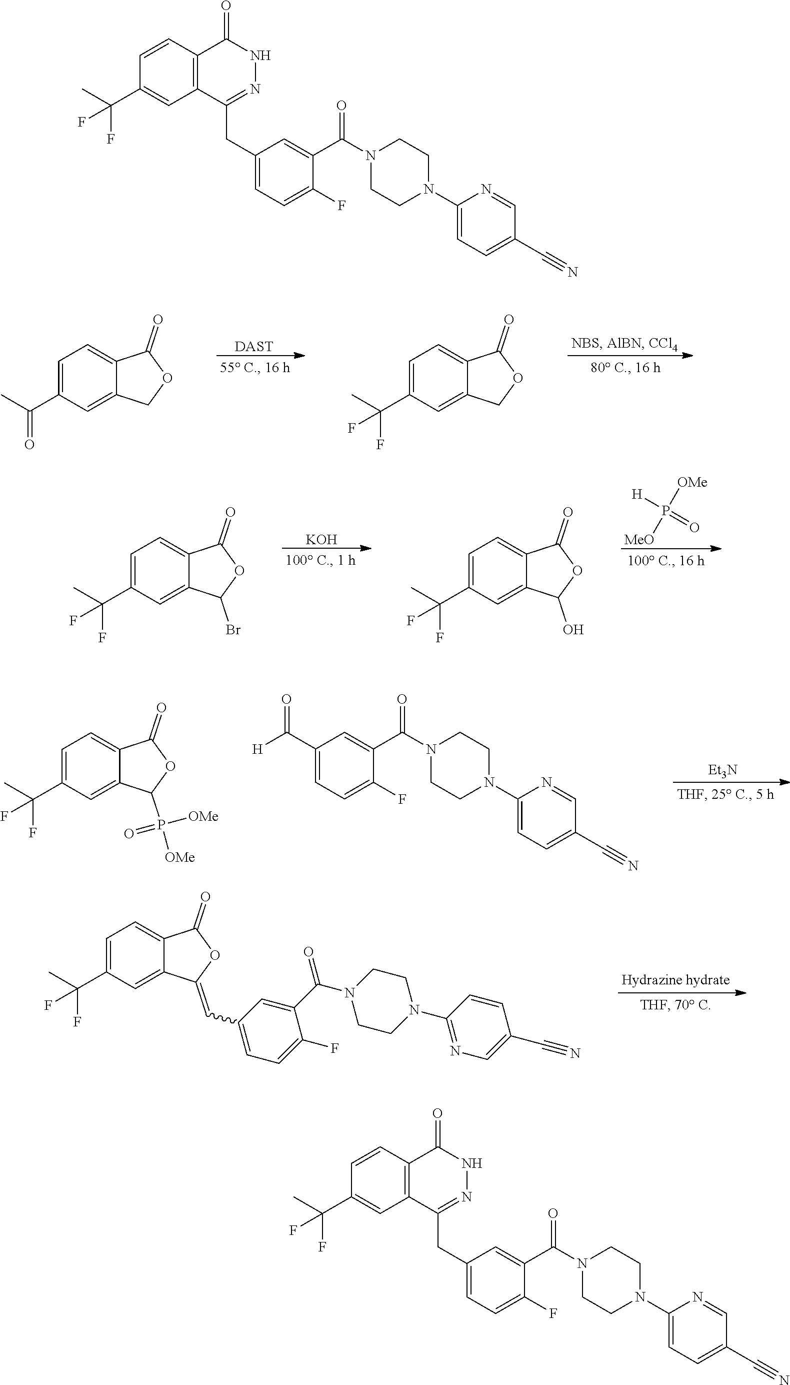

### Step 1

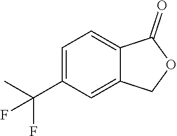

### 5-(1,1-Difluoroethyl)isobenzofuran-1(3H)-one

To a solution of 5-acetyl-3H-isobenzofuran-1-one (950 mg, 5.39 mmol) in DCM (20 mL) was added DAST (8.69 g, 53.9 mmol, 7.12 mL). The reaction mixture was stirred at 55° C. for 16 hours. The mixture was poured to sat. NaHCO₃, extracted with DCM (20 mL×3). The extracts were washed with sat, brine, dried over Na₂SO₄, filtered. The organic layer was concentrated to give the crude. The crude was purified by silica gel chromatography to give 5-(1,1-difluoroethyl)-3H-isobenzofuran-1-one (400 mg, 37% yield) as a grey solid. ¹H NMR (400 MHz, DMSO-dL) S 7.97 (d, J=8.0 Hz, 1H), 7.91 (d, J=0.7 Hz, 1H), 7.78 (dd, J=8.0, 0.6 Hz, 1H), 5.47 (s, 2H), 2.03 (t, J=19.0 Hz, 3H).

### Step 2

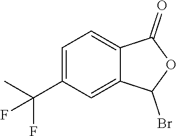

### 5-(1,1-Difluoroethyl)isobenzofuran-1(3H)-one

To a solution of 5-(1,1-difluoroethyl)-3H-isobenzofuran-1-one (350 mg, 1.77 mol) in CCl₄ (20 mL) was added NBS (472 mg, 2.65 mmol, 225 μL) and AIBN (29.0 mg, 177 μmol). The mixture was stirred at 80° C. for 16 hours. The mixture was purified by silica gel chromatography (petroleum ether:EtOAc=4:1) to give 3-bromo-5-(1,1-difluoroethyl)-3H-isobenzofuran-1-one (420 mg, 86% yield) as a light yellow oil. ¹H NMR (400 MHz, CDCl₃) δ 7.99 (d, J=7.9 Hz, 1H), 7.76 (d, J=8.8 Hz, 2H), 7.42 (s, 1H), 1.98 (t, J=18.2 Hz, 3H).

### Step 3

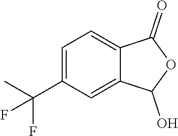

### 5-(1,1-Difluoroethyl)-3-hydroxyisobenzofuran-1(3H)-one

To a 50 mL round bottle flask was added 3-bromo-5-(1,1-difluoroethyl)-3H-isobenzofuran-1-one (420 mg, 1.52 mmol) and KOH (1 M, 20 mL). The mixture was stirred at 100° C. for 1 hour. The mixture was acidified to pH 5 with 1N HCl, extracted with DCM (20 mL×3). The extracts were washed with brine, dried over Na₂SO₄, filtered and concentrated to give 5-(1,1-difluoroethyl)-3-hydroxy-3H-isobenzofuran-1-one as a light yellow solid (290 mg, 89% yield). LCMS ESI m/z: 212.9 [M+H]⁺.

### Step 4

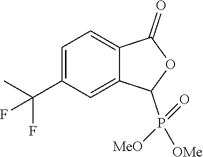 [SMILES: COP(=O)(OC)C1OC(=O)c2ccc(C(C)(F)F)cc21]

### Dimethyl 6-(1,1-difluoroethyl)-3-oxo-1,3-dihydroisobenzofuran-1-ylphosphonate

To a 50 mL round bottle flask was added 5-(1,1-difluoroethyl)-3-hydroxy-3H-isobenzofuran-1-one (290 mg, 1.35 mmol) and dimethylphosphite (149 mg, 1.35 mmol, 5 mL). The mixture was stirred at 100° C. for 16 hours. The mixture was poured into water (50 mL), extracted with DCM (30 mL×3), dried over Na₂SO₄, filtered and concentrated to the residue. The residue was purified by silica gel chromatography (PE:EA=10:1) to give dimethyl 6-(1,1-difluoroethyl)-3-oxo-1,3-dihydroisobenzofuran-1-ylphosphonate (810 mg, crude) as a light yellow oil. LCMS ESI m/z: 307.1 [M+H]⁺.

### Step 5

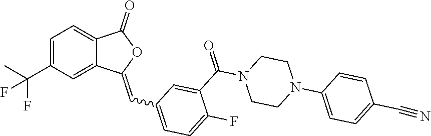 [SMILES: CC(F)(F)c1ccc2c(c1)C(=Cc1ccc(F)c(C(=O)N3CCN(c4ccc(C#N)cc4)CC3)c1)OC2=O]

### 6-(4-(5-((6-(1,1-Difluoroethyl)-3-oxoisobenzofuran-1(3H)-ylidene)methyl)-2-fluorobenzoyl)piperazin-1-yl)nicotinonitrile

To a solution of dimethyl 6-(1,1-difluoroethyl)-3-oxo-1,3-dihydroisobenzofuran-1-ylphosphonate (442 mg, 1.31 mmol) in THF (15 mL) was added ET3N (397 mg, 3.92 mmol, 546 μL) and 6-(4-(2-fluoro-5-formylbenzoyl)piperazin-1-yl)nicotinonitrile (800 mg, 1.31 mmol), The mixture was stirred at rt for 16 hours. The mixture was poured into water (30 mL), extracted with DCM (50 mL×3). The organic layers were dried over Na₂SO₄, filtered and concentrated to the crude. The crude was purified by silica gel chromatography (PE:EA=1:1) to give 6-[4-[5-[[6-(1,1-difluoroethyl)-3-oxo-isobenzofuran-1-ylidene]methyl]-2-fluoro-benzoyl]piperazin-1-yl]pyridine-3-carbonitrile (290 mg, 43% yield) as a light yellow oil. LCMS ESI m/z: 519.2 [M+H]⁺.

### Step 6

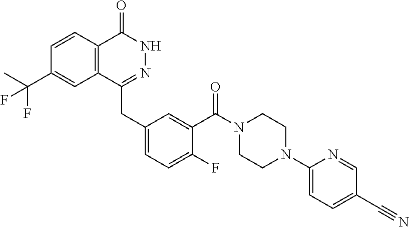 [SMILES: CC(F)(F)c1ccc2c(=O)[nH]nc(Cc3ccc(F)c(C(=O)N4CCN(c5ccc(C#N)cn5)CC4)c3)c2c1]

### 6-(4-(5-((7-(1,1-Difluoroethyl)-4-oxo-3,4-dihydrophthalazin-1-yl)methyl)-2-fluorobenzoyl)piperazin-1-yl)nicotinonitrile

To a solution of 6-[4-[5-[[6-(1,1-difluoroethyl)-3-oxo-isobenzofuran-1-ylidene]methyl]-2-fluoro-benzoyl]piperazin-1-yl]pyridine-3-carbonitrile (290 mg, 559 μmol) in THF (4 mL) was added N₂H₄·H₂O (2 mL). The mixture was stirred at 80° C. for 3 hours. The mixture was purified by Prep-HPLC to give 6-(4-(5-((7-(1,1-difluoroethyl)-4-oxo-3,4-dihydrophthalazin-1-yl)methyl)-2-fluorobenzoyl)piperazin-1-yl)nicotinonitrile (101 mg, 34% yield) as a white solid. LCMS ESI m/z: 532.8 [M+H]⁺. ¹H NMR (400 MHz, DMSO-d₆) δ 12.73 (s, 1H), 8.51 (d, J=2.2 Hz, 1H), 8.37 (d, J=8.3 Hz, 1H), 8.06 (s, 1H), 8.00 (d, J=8.4 Hz, 1H), 7.90 (dd, J=9.1, 2.3 Hz, 1H), 7.49-7.40 (m, 2H), 7.26 (t, J=9.0 Hz, 1H), 6.91 (d, J=9.1 Hz, 1H), 4.42 (s, 2H), 3.75 (m, 4H), 3.60 (m, 2H), 3.30 (m, 2H), 2.03 (t, J=19.1 Hz, 3H).

### Synthesis of Example 37: 6-(4-(5-((7-(Cyclopropylethynyl)-4-oxo-3,4-dihydrophthalazin-1-yl)methyl)-2-fluorobenzoyl)piperazin-1-yl)nicotinonitrile

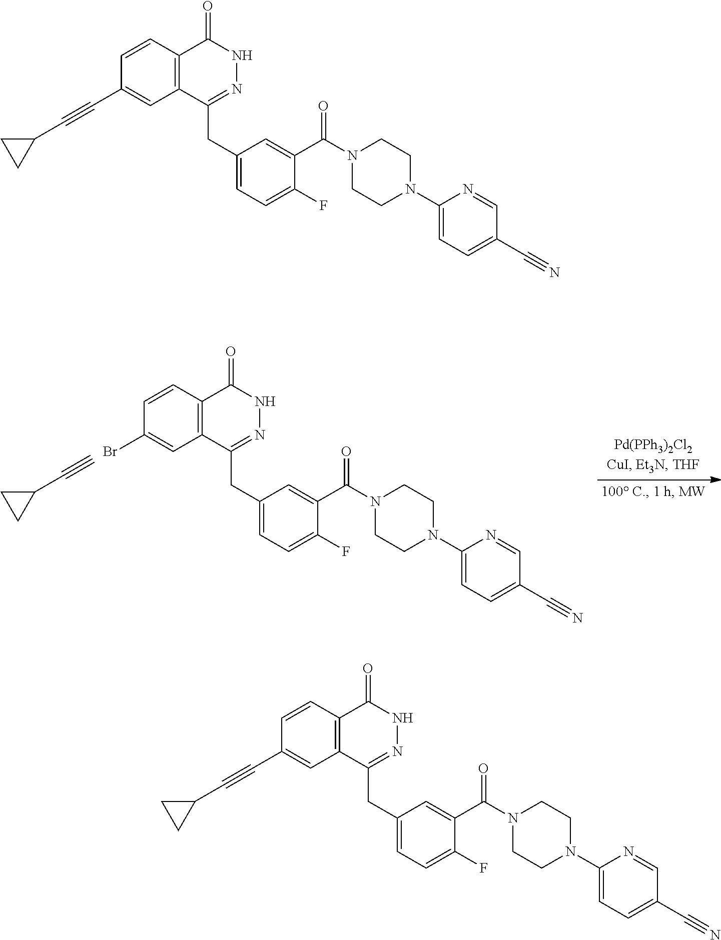

### Step 1

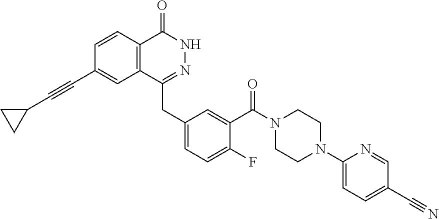 [SMILES: N#Cc1ccc(N2CCN(C(=O)c3cc(Cc4n[nH]c(=O)c5ccc(C#CC6CC6)cc45)ccc3F)CC2)nc1]

### 6-(4-(5-((7-(Cyclopropylethynyl)-4-oxo-3,4-dihydrophthalazin-1-yl)methyl)-2-fluorobenzoyl)piperazin-1-yl)nicotinonitrile

To a mixture of 6-[4-[5-[(7-bromo-4-oxo-3H-phthalazin-1-yl)methyl]-2-fluoro-benzoyl]piperazin-1-yl]pyridine-3-carbonitrile (50.0 mg, 91.3 μmol) in THF (1.5 mL) was added ethynylcyclopropane (12.1 mg, 183 μmol, 15.46 μL), CuI (8.70 mg, 45.7 μmol, 1.55 μL), Pd(PPh₃)₂Cl₂ (6.41 mg, 9.13 μmol), Et₃N (27.7 mg, 274 μmol, 38.2 μL). The reaction mixture was stirred under microwave at 110° C. for 1 hour. The mixture was purified by Prep-HPLC to give 6-(4-(5-((7-(cyclopropylethynyl)-4-oxo-3,4-dihydrophthalazin-1-yl)methyl)-2-fluorobenzoyl)piperazin-1-yl)nicotinonitrile (8.43 mg, 17% yield) as a white solid. LCMS ESI m/z: 532.8 [M+H]⁺. ¹H-NMR (400 MHz, DMSO-d₆) δ 12.61 (s, 1H), 8.51 (d, J=2.2 Hz, 1H), 8.18 (d, J=8.2 Hz, 1H), 7.90 (dd, J=9.3, 2.5 Hz, 2H), 7.77-7.72 (m, 1H), 7.41 (d, J=5.7 Hz, 2H), 7.27 (d, J=9.5 Hz, 1H), 6.92 (d, J=9.1 Hz, 1H), 4.33 (s, 2H), 3.75 (m, 4H), 3.63 (m, 2H), 3.33 (m, 2H), 1.58 (s, 1H), 0.97-0.86 (m, 2H), 0.84-0.70 (m, 2H).

### Synthesis of Example 38: 6-(4-(2-Fluoro-5-((4-oxo-7-(pyridin-2-yloxy)-3,4-dihydrophthalazin-1-yl)methyl)benzoyl)piperazin-1-yl)nicotinonitrile

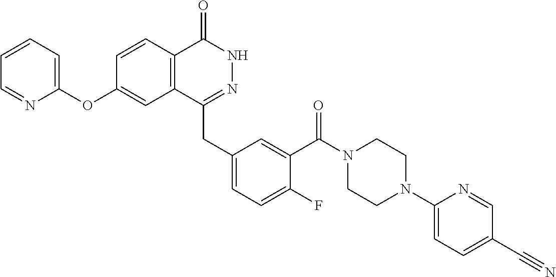 [SMILES: N#Cc1ccc(N2CCN(C(=O)c3cc(Cc4n[nH]c(=O)c5ccc(Oc6ccccn6)cc45)ccc3F)CC2)nc1]

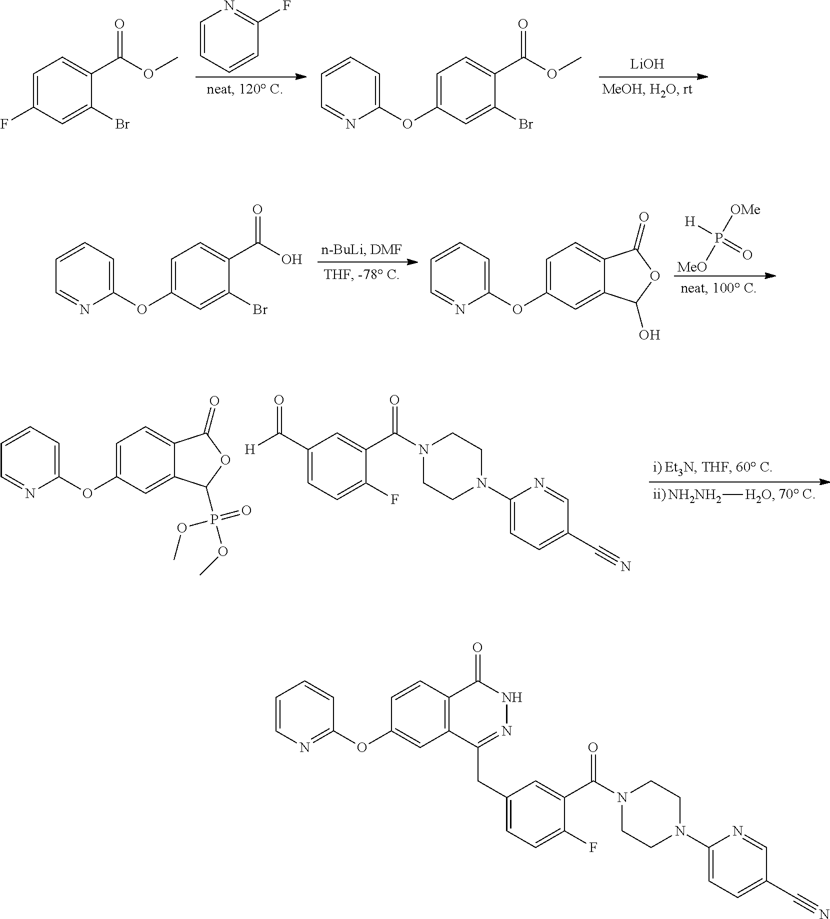

### Step 1

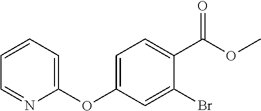 [SMILES: COC(=O)c1ccc(Oc2ccccn2)cc1Br]

### Methyl 2-bromo-4-(pyridin-2-yloxy)benzoate

A mixture of methyl 2-bromo-4-fluorobenzoate (2.50 g, 10.82 mmol) and 2-fluoropyridine (1 mL) was stirred at 120° C. for 16 hours. The reaction was cooled to rt, diluted with H₂O (100 mL) and extracted with EtOAc (100 mL×3). The combined organic layer was washed with brine, dried over anhydrous Na₂SO₄, filtered and concentrated in vacuum. The residue was purified by silica gel chromatography (EtOAc/petroleum ether=0% to 7%) to give methyl 2-bromo-4-(pyridin-2-yloxy)benzoate (2.83 g, 85% yield) as a colorless oil. LCMS ESI m/z: 308.0 [M+H]⁺.

### Step 2

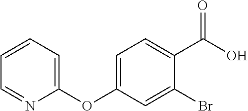 [SMILES: O=C(O)c1ccc(Oc2ccccn2)cc1Br]

### 2-Bromo-4-(pyridin-2-yloxy)benzoic acid

A mixture of methyl 2-bromo-4-(pyridin-2-yloxy)benzoate (2.83 g, 9.18 mmol) and NaOH (1.84 g, 45.92 mmol) in MeOH (30 mL) and H₂O (10 mL) was stirred at rt for 2 hours. The organic solvent was removed in vacuo and aq HCl (1N) was added until the solution reached pH 5˜6. The formed solid was collected by filtration, washed with H₂O, dried under vacuum to give 2-bromo-4-(pyridin-2-yloxy)benzoic acid (2.2 g, 81% yield) as a white solid. LCMS ESI m/z: 294.0 [M+H]⁺.

### Step 3

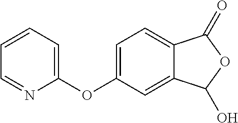 [SMILES: O=C1OC(O)c2cc(Oc3ccccn3)ccc21]

### 3-Hydroxy-5-(pyridin-2-yloxy)isobenzofuran-1(3H)-one

A solution of 2-bromo-4-(pyridin-2-yloxy)benzoic acid (1.90 g, 6.46 mmol) in THF (30 mL) was cooled to −78° C. then n-BuLi (2.5 M in heptane, 5.7 mL, 14.25 mmol) was added dropwise under Ar (g). The reaction was stirred at −78° C. for 30 min, then added DMF (1.05 g, 14.33 mmol), The reaction was stirred at −78° C. for 20 min, then quenched with sat. NH₄Cl solution (10 mL), neutralized by aq HCl (1N) until the solution reached pH 5˜6, and extracted with EtOAc (30×3 mL). The combined organic layer was washed with bine, dried over Na₂SO₄, filtered and concentrated in vacuum. The residue was purified by silica gel chromatography (EtOAc/petroleum ether=30% to 50%) to give 3-hydroxy-5-(pyridin-2-yloxy)isobenzofuran-1(3H)-one (680 mg, 43% yield) as a white solid. LCMS ESI m/z: 244.1 [M+H]⁺.

### Step 4

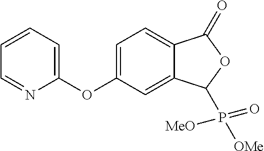 [SMILES: COP(=O)(OC)C1OC(=O)c2ccc(Oc3ccccn3)cc21]

### Dimethyl (3-oxo-6-(pyridin-2-yloxy)-1,3-dihydroisobenzofuran-1-yl)phosphonate

A solution of 3-hydroxy-5-(pyridin-2-yloxy)isobenzofuran-1(3H)-one (480 mg, 1.97 mmol) in dimethyl phosphonate (5 mL) was stirred at 100° C. for 16 hours. The mixture was cooled to rt, diluted with H₂O (10 mL) and extracted with EtOAc (10 mL×3). The combined organic layer was washed with brine, dried over anhydrous Na₂SO₄, filtered and concentrated in vacuo. The residue was purified by silica gel chromatography (EtOAc/petroleum ether=50% to 60%) to give dimethyl (3-oxo-6-(pyridin-2-yloxy)-1,3-dihydroisobenzofuran-1-yl)phosphonate (20) mg, 30% yield) as a white solid. LCMS ESI m/z: 334.1 [M+H]⁺.

### Step 5

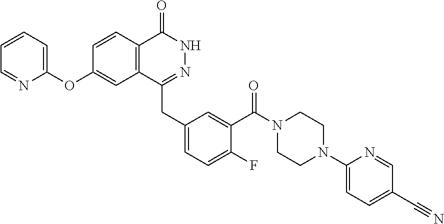 [SMILES: N#Cc1ccc(N2CCN(C(=O)c3cc(Cc4n[nH]c(=O)c5ccc(Oc6ccccn6)cc45)ccc3F)CC2)nc1]

### 6-(4-(2-Fluoro-5-((4-oxo-7-(pyridin-2-yloxy)-3,4-dihydrophthalazin-1-yl)methyl)benzoyl)piperazin-1-yl)nicotinonitrile

A solution of dimethyl (3-oxo-6-(pyridin-2-yloxy)-1,3-dihydroisobenzofuran-1-yl)phosphonate (200 mg, 0.59 mmol), 6-(4-(2-fluoro-5-formylbenzoyl)piperazin-1-yl)nicotinonitrile (212 mg, 0.63 mmol) and Et₃N (181 mg, 1.79 mmol) in anhydrous THF (2 mL) was stirred at 60° C. under Ar (g) for 15 hours. Hydrazine hydrate (46 mg, 0.78 mmol) was added and the reaction was stirred at 70° C. for 1 hour. The reaction was cooled to rt, then THF was removed under vacuum. The formed solid was filtered, washed with water (10 mL) and petroleum ether/EtOAc (2:1) to give 6-(4-(2-fluoro-5-((4-oxo-7-(pyridin-2-yloxy)-3,4-dihydrophthalazin-1-yl)methyl)benzoyl)piperazin-1-yl)nicotinonitrile (60 mg, 20% yield) as a white solid. LCMS ESI m/z: 562.0 [M+H]⁺. ¹H NMR (400 MHz, DMSO-d₆) δ 12.60 (s, 1H), 8.51 (d, J=2.2 Hz, 1H), 8.28 (d, J=8.7 Hz, 1H), 8.17-8.13 (m, 1H), 7.95-7.88 (m, 2H), 7.67 (d, J=2.1 Hz, 1H), 7.58-7.53 (m, 1H), 7.44-7.34 (m, 2H), 7.27-7.14 (m, 3H), 6.89 (d, J=9.1 Hz, 1H), 4.28 (s, 2H), 3.73 (m, 4H), 3.56 (m, 2H), 3.26 (m, 2H).

### Synthesis of Example 39: 6-(4-(2-Fluoro-5-((7-(naphthalen-2-yloxy)-4-oxo-3,4-dihydrophthalazin-1-yl)methyl)benzoyl)piperazin-1-yl)nicotinonitrile

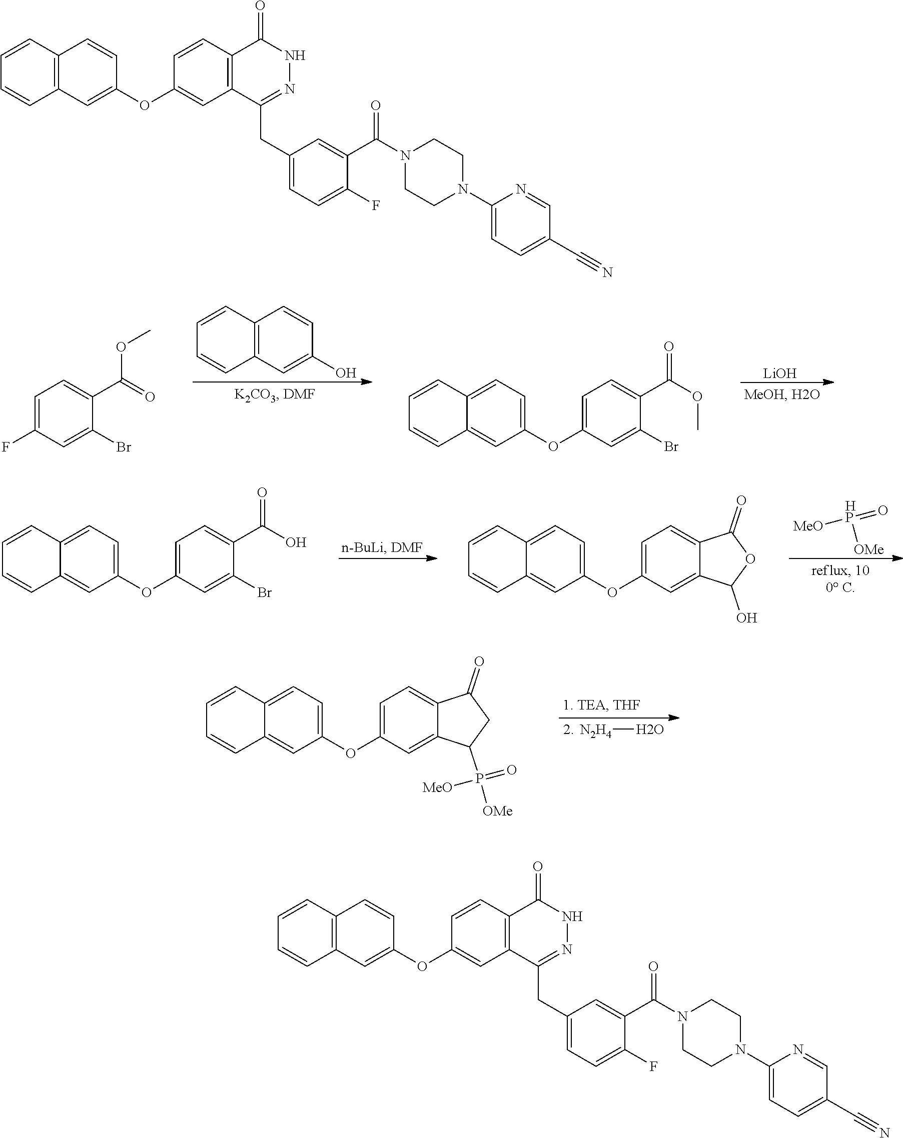

### Step 1

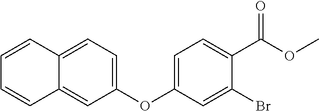 [SMILES: COC(=O)c1ccc(Oc2ccc3ccccc3c2)cc1Br]

### Methyl 2-bromo-4-(naphthalen-2-yloxy)benzoate

A mixture of ethyl methyl 2-bromo-4-fluorobenzoate (3.00 g, 12.9 mmol), naphthalen-2-ol (2.78 g, 19.3 mmol) and K₂CO₃ (3.56 g, 25.8 mmol) in DMF (30 mL) was stirred at 100° C. for 16 hours. The mixture was diluted with H₂O (60 mL) and extracted with EtOAc (3×50 mL). The combined organic layer was dried over Na₂SO₄, filtered and concentrated in vacuo. The residue was purified by silica gel chromatography (EtOAc/petroleum ether=0% to 50%) to give methyl 2-bromo-4-(naphthalen-2-yloxy)benzoate (4.00 g, 87% yield) as a white solid. LCMS ESI m/z: 357 [M+H]+.

### Step 2

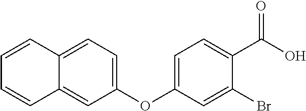 [SMILES: O=C(O)c1ccc(Oc2ccc3ccccc3c2)cc1Br]

### 2-Bromo-4-(naphthalen-2-yloxy)benzoic acid

A mixture of methyl 2-bromo-4-(naphthalen-2-yloxy)benzoate (4.00 g, 11.2 mmol) and NaOH (2.24 g, 56.0 mmol) in MeOH (15 mL) and H₂O (5 mL) was stirred at rt for 2 hours. The organic solvent was removed in vacuo and aq HCl (1N) was added until the solution reached pH 5˜6. The solids were collected by filtration, washed with water and dried under vacuum to give 2-bromo-4-(naphthalen-2-yloxy)benzoic acid (3.5 g, 91% yield) as a white solid. LCMS ESI n/z: 343 [M+H]⁺.

### Step 3

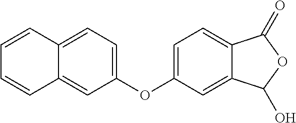 [SMILES: O=C1OC(O)c2cc(Oc3ccc4ccccc4c3)ccc21]

### 3-Hydroxy-5-(naphthalen-2-yloxy)isobenzofuran-1(3H)-one

A solution of 2-bromo-4-(naphthalen-2-yloxy)benzoic acid (1.00 g, 2.91 mmol) in THF (30 mL) was cooled to −78° C., then n-BuLi (2.5 M in heptane, 2.33 mL, 5.83 mmol) was added dropwise under Ar (g). The reaction was stirred at −78° C. for 30 min, then added DMF (469 mg, 6.41 mmol), The reaction was stirred at −78° C. for 20 min, then quenched with sat. NH₄Cl solution (10 mL) and added aq HCl (1M) until the solution reached pH 5˜6. The mixture was extracted with EtOAc (30×3 mL). The combined organic layer was washed with brine, dried over Na₂SO₄, filtered and concentrated in vacuo. The residue was purified by silica gel chromatography (EtOAc/petroleum ether=0% to 20%) to give 3-hydroxy-5-(naphthalen-2-yloxy)isobenzofuran-1(3H)-one (540 mg, 63% yield) as a white solid. LCMS ESI m/z: 293 [M+H]⁺.

### Step 4

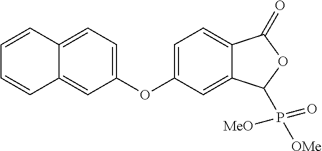 [SMILES: COP(=O)(OC)C1OC(=O)c2ccc(Oc3ccc4ccccc4c3)cc21]

### Dimethyl (6-(naphthalen-2-yloxy)-3-oxo-1,3-dihydroisobenzofuran-1-yl)phosphonate

A solution of 3-hydroxy-5-(naphthalen-2-yloxy)isobenzofuran-1(3H)-one (540 mg, 1.85 mmol) in dimethyl phosphonate (10 mL) was stirred at 100° C. for 16 hours. The mixture was cooled to it, diluted with H₂O (20 mL) and extracted with EtOAc (20 mL×3). The combined organic layer was washed with brine, dried over anhydrous Na₂SO₄, filtered and concentrated in vacuo to give dimethyl (6-(naphthalen-2-yloxy)-3-oxo-1,3-dihydroisobenzofuran-1-yl)phosphonate (690 mg, 97% yield) as a light yellow oil. LCMS ESI m/z: 385 [M+H]⁺.

### Step 5

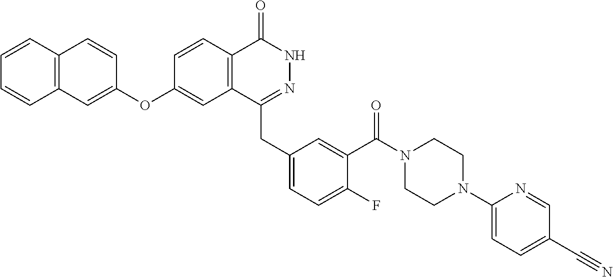 [SMILES: N#Cc1ccc(N2CCN(C(=O)c3cc(Cc4n[nH]c(=O)c5ccc(Oc6ccc7ccccc7c6)cc45)ccc3F)CC2)nc1]

### 6-(4-(2-fluoro-5-((7-(naphthalen-2-yloxy)-4-oxo-3,4-dihydrophthalazin-1-yl)methyl)benzoyl)piperazin-1-yl)nicotinonitrile

A solution of dimethyl (6-(naphthalen-2-yloxy)-3-oxo-1,3-dihydroisobenzofuran-1-yl)phosphonate (200 mg, 0.52 mmol), 6-(4(2-fluoro-5-formylbenzoyl)piperazin-1-yl)nicotinonitrile (176 mg, 0.52 mmol) and Et₃N (158 mg, 1.56 mmol) in THF (5 mL) was stirred at 25° C. under Ar (g) for 15 hours. Hydrazine hydrate (46 mg, 0.78 mmol) was added and the reaction was stirred at 70° C. for 1 hour. The reaction was cooled to rt, and the solvent was removed under vacuum. The formed solids were washed with water (10 mL) and petroleum ether/EA (2:1, 10 mL) to give 6-(4-(2-fluoro-5-((7-(naphthalen-2-yloxy)-4-oxo-3,4-dihydrophthalazin-1-yl)methyl)benzoyl)piperazin-1-yl)nicotinonitrile (119 mg, 37% yield) as a white solid. LCMS ESI m/z: 611 [M+H]+. ¹H NMR (400 MHz, DMSO-d₆) δ 12.58 (s, 1H), 8.49-8.44 (m, 1H), 8.27 (d, J=8.7 Hz, 1H), 8.05-7.97 (m, 2H), 7.88-7.81 (m, 2H), 7.59-7.57 (m, 1H), 7.56-7.50 (m, 2H), 7.50-7.48 (m, 1H), 7.48-7.44 (m, 1H), 7.35-7.30 (m, 2H), 7.29-7.24 (m, 1H), 7.06 (t, J=9.0 Hz, 1H), 6.84 (d, J=9.1 Hz, 1H), 4.23 (s, 2H), 3.78-3.68 (m, 4H), 3.60-3.55 (m, 2H), 3.27-3.23 (m, 2H).

### Synthesis of Example 40: 6-(4-(5-((7-Cyclopropyl-4-oxo-3,4-dihydrophthalazin-1-yl)methyl)-2-fluorobenzoyl)piperazin-1-yl)nicotinonitrile

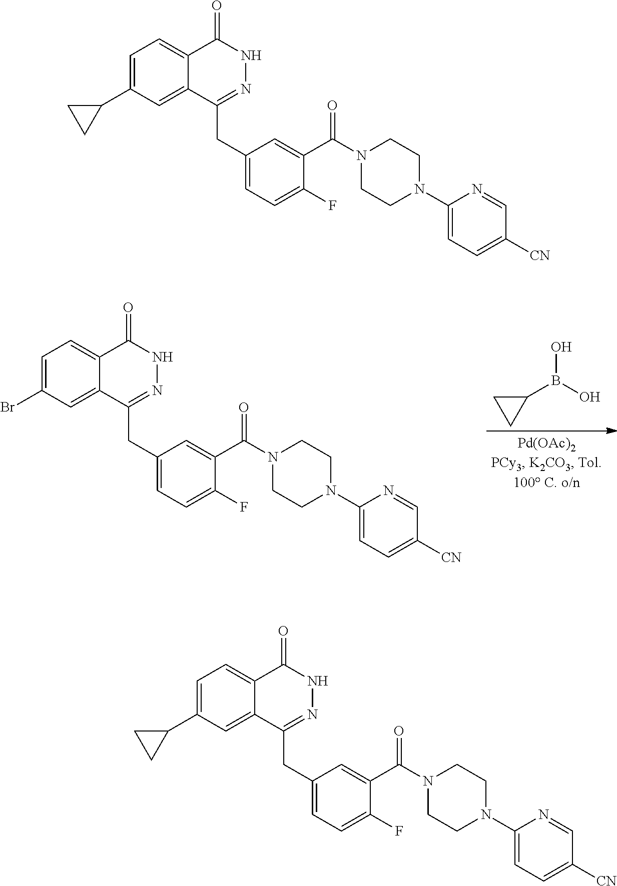 [SMILES: N#Cc1ccc(N2CCN(C(=O)c3cc(Cc4n[nH]c(=O)c5ccc(C6CC6)cc45)ccc3F)CC2)nc1 ; N#Cc1ccc(N2CCN(C(=O)c3cc(Cc4n[nH]c(=O)c5ccc(Br)cc45)ccc3F)CC2)nc1 ; OB(O)C1CC1 ; N#Cc1ccc(N2CCN(C(=O)c3cc(Cc4n[nH]c(=O)c5ccc(C6CC6)cc45)ccc3F)CC2)nc1]

### Step 1

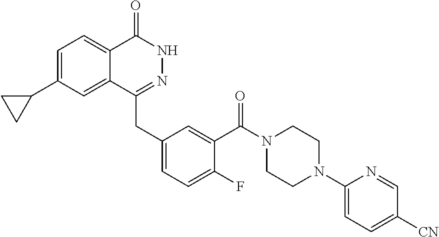 [SMILES: N#Cc1ccc(N2CCN(C(=O)c3cc(Cc4n[nH]c(=O)c5ccc(C6CC6)cc45)ccc3F)CC2)nc1]

### 6-(4-(5-((7-Cyclopropyl-4-oxo-3,4-dihydrophthalazin-1-yl)methyl)-2-fluorobenzoyl)piperazin-1-yl)nicotinonitrile

6-(4-(5-((7-bromo-4-oxo-3,4-dihydrophthalazin-1-yl)methyl)-2-fluorobenzoyl)piperazin-1-yl)nicotinonitrile (27.7 mg, 0.05 mmol), cyclopropylboronic acid (13.0 mg, 0.15 mmol), Pd(OAc)₂ (3.0 mg, 0.013 mmol), PCy₃ (8.0 mg, 0.029 mmol), K₂CO₃ (21.0 mg, 0.15 mmol), and toluene were added in a pressure vial under N₂ atmosphere. The mixture was heated at 100° C. overnight. The crude residue was purified by flash (0-10(0% EtOAc in hexanes) to afford the product as a white solid (4.3 mg, 16% yield). LCMS ESI m/z: 509.2 [M+H]^{+ 1}H NMR (400 MHz, CDCl₃) δ 10.22 (s, 1H), 8.41 (d, J=1.9 Hz, 1H), 8.32 (d, J=8.2 Hz, 1H), 7.42-7.30 (m, 4H), 7.08 (d, J=8.8 Hz, 1H), 6.61 (d, J=8.9 Hz, 1H), 4.27 (s, 2H), 3.89 (s, 2H), 3.77 (s, 2H), 3.73-3.60 (m, 2H), 3.44 (s, 2H), 2.01 (m, 1H), 1.26 (m, 2H), 1.16-1.09 (m, 2H).

### Synthesis of Example 41: 6-(4-(2-Fluoro-5-((7-neopentyl-4-oxo-3,4-dihydrophthalazin-1-yl)methyl)benzoyl)piperazin-1-yl)nicotinonitrile

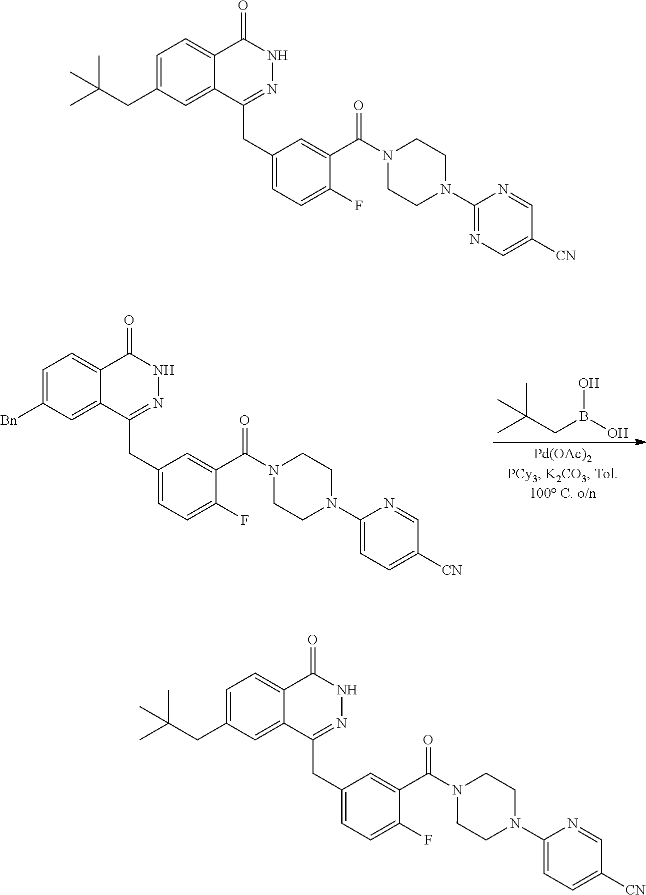

### Step 1

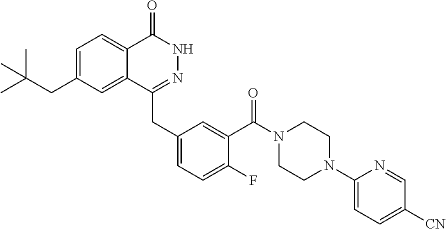 [SMILES: CC(C)(C)Cc1ccc2c(=O)[nH]nc(Cc3ccc(F)c(C(=O)N4CCN(c5ccc(C#N)cn5)CC4)c3)c2c1]

### 6-(4-(2-Fluoro-5-((7-neopentyl-4-oxo-3,4-dihydrophthalazin-1-yl)methyl)benzoyl)piperazin-1-yl)nicotinonitrile

6-(4-(5-((7-Bromo-4-oxo-3,4-dihydrophthalazin-1-yl)methyl)-2-fluorobenzoyl)piperazin-1-yl)nicotinonitrile (27.7 mg, 0.05 mmol), neopentylboronic acid (0.017 g, 0.15 mmol), Pd(OAc)₂ (0.0 mg, 0.013 mmol), PCy₃ (8.0 mg, 0.029 mmol), K₂CO₃ (21.0 mg, 0.15 mmol), and toluene were added in a pressure vial under N₂ atmosphere. The mixture was heated at 100° C. overnight. The reaction was cooled to rt and concentrated in vacuo. The crude residue was purified via a flash chromatography (0-100% EtOAc in hexanes) to afford the product as a white solid (5.4 mg, 19% yield). LC-MS ESI m/z 539.4 [M+H]⁺. ¹H NMR (400 MHz, CDCl₃) δ 9.91 (s, 1H), 8.35 (d, J=1.7 Hz, 1H), 8.28 (d, J=8.1 Hz, 1H), 7.59 (dd, J=9.0, 2.3 Hz, 1H), 7.46 (dd, =8.1, 1.5 Hz, 1H), 7.35 (s, 1H), 7.29 (m, 2H), 6.98 (t, J=8.8 Hz, 1H), 6.55 (d, J=9.0 Hz, 1H), 4.21 (s, 2H), 3.82 (s, 2H), 3.70 (s, 2H), 3.62 (s, 2H), 3.34 (s, 2H), 1.98 (s, 2H), 0.95 (s, 9H).

### Synthesis of Example 42: 4-(4-Fluoro-3-(4-(5-(trifluoromethyl)pyrimidin-2-yl)piperazine-1-carbonyl)benzyl)-6-(3,3,3-trifluoroprop-1-yn-1-yl)phthalazin-1(2H)-one

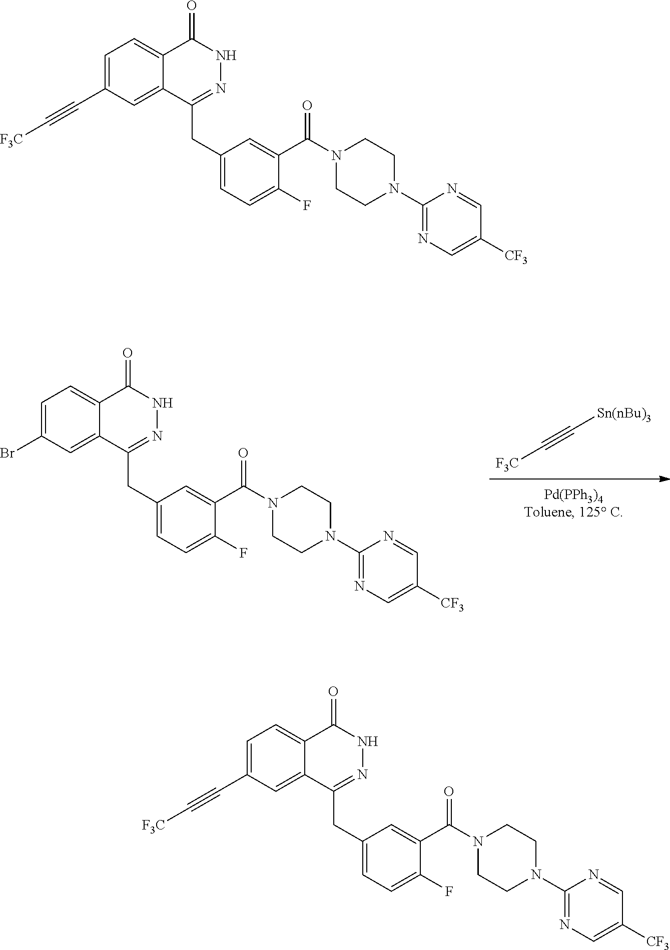

### Step 1

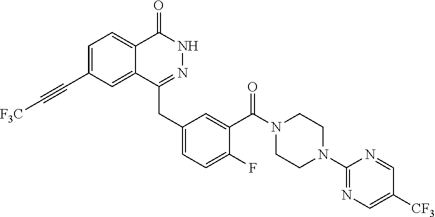 [SMILES: O=C(c1cc(Cc2n[nH]c(=O)c3ccc(C#CC(F)(F)F)cc23)ccc1F)N1CCN(c2ncc(C(F)(F)F)cn2)CC1]

### 4-(4-Fluoro-3-(4-(5-(trifluoromethyl)pyrimidin-2-yl)piperazine-1-carbonyl)benzyl)-6-(3,3,3-trifluoroprop-1-yn-1-yl)phthalazin-1(2H)-one

6-Bromo-4-(4-fluoro-3-(4-(5-(trifluoromethyl)pyrimidin-2-yl)piperazine-1-carbonyl)benzyl)phthalazin-1(2H)-one (24.0 mg, 0.04 mmol) was dissolved in anhydrous toluene (0.5 mL) under argon gas and the solution was degassed with argon for 5 min. Palladium tetrakis(triphenylphosphine) (7.0 mg, 0.00583 mmol.) was added quickly and the solution degassed with argon and stirred for 10 min. To this mixture tributyl(3,3,3-trifluoroprop-1-yn-1-yl)stannane (0.025 uL, 0.06 mmol) was added dropwise. The reaction mixture was heated at 125° C. for 4 h in a sealed tube. The reaction mixture was cooled to rt and concentrated in vacuo. The crude residue was purified via flash chromatography (0-80% EtOAc in hexanes) to afford the product as a white solid (6.6 mg, 27% yield). LCMS ESI m/z: 605.0 [M+H]⁺. ¹H NMR (400 MHz, CDCl₃) 9.78 (s, 1H), 8.33 (s, 2H), 8.13 (d, J=8.9 Hz, 1H), 7.74-7.65 (m, 2H), 7.11-7.16 (m, 2H), 6.91 (t, J=8.8 Hz, 1H), 4.06 (s, 2H), 3.84 (s, 2H), 3.74-3.67 (m, 4H), 3.22 (s, 2H).

### Synthesis of Example 43: 6-Ethyl-4-(4-fluoro-3-(4-(5-(trifluoromethyl)pyrimidin-2-yl)piperazine-1-carbonyl)benzyl)phthalazin-1(2H)-one

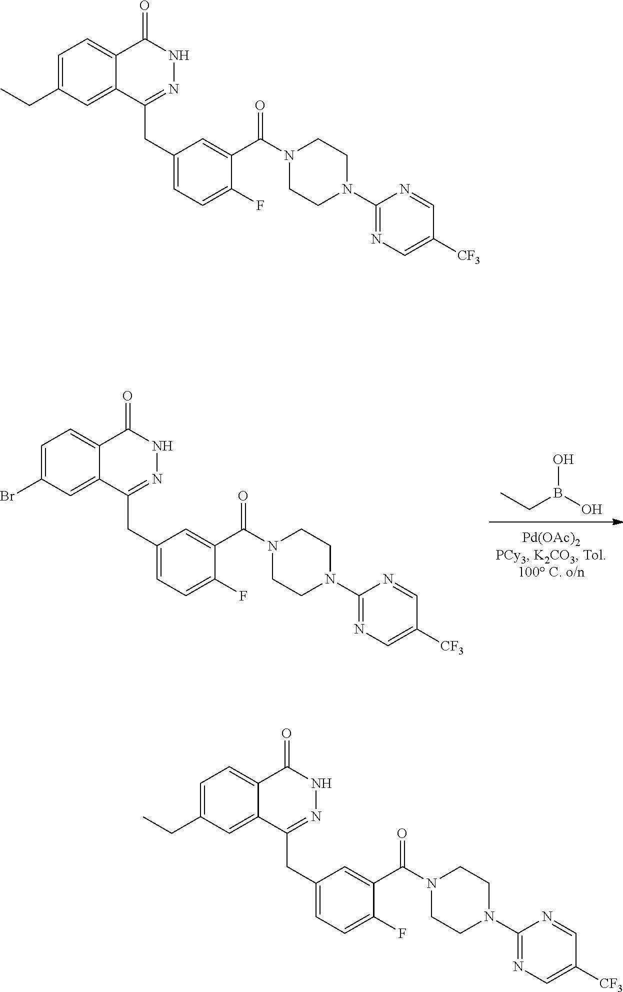

### Step 1

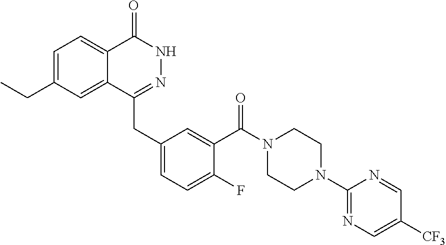 [SMILES: CCc1ccc2c(=O)[nH]nc(Cc3ccc(F)c(C(=O)N4CCN(c5ncc(C(F)(F)F)cn5)CC4)c3)c2c1]

### 6-Ethyl-4-(4-fluoro-3-(4-(5-(trifluoromethyl)pyrimidin-2-yl)piperazine-1-carbonyl)benzyl)phthalazin-1(2H)-one

6-Bromo-4-(4-fluoro-3-(4-(5-(trifluoromethyl)pyrimidin-2-yl)piperazine-1-carbonyl)benzyl)phthalazin-1(2H)-one (0.030 g, 0.05 mmol), ethylboronic acid (0.008 g, 3.0 equiv). Pd(OAc)₂ (2 mg), PCy₃ (4 mg), K₂CO₃ (21 mg) and toluene were added in a pressure vial under N₂ atmosphere. The mixture was heated at 100° C. overnight. The reaction was cooled to rt and concentrated in vacuo. The crude residue was purified via flash chromatography (0-100% EtOAc in hexanes) to afford the product as a white solid (8.4 mg, 31% yield). LCMS ESI m/z: 540.4 [M+H]⁺. ¹H NMR (400 MHz, CDCl₃) δ 10.12 (s, 1H), 8.43 (s, 2H), 8.26 (d, J=7.9 Hz, 1H), 7.34-7.27 (m, 4H), 7.00 (t, J=8.8 Hz, 1H), 4.19 (s, 2H), 3.92-3.77 (m, 4H), 3.32 (s, 2H), 2.75 (q, J=7.2 Hz, 1H), 1.23 (t, J=7.2 Hz, 1H).

### Synthesis of Example 44: 4-(4-Fluoro-3-(4-(5-(trifluoromethyl)pyrimidin-2-yl)piperazine-1-carbonyl)benzyl)-6-isobutylphthalazin-1(2H)-one

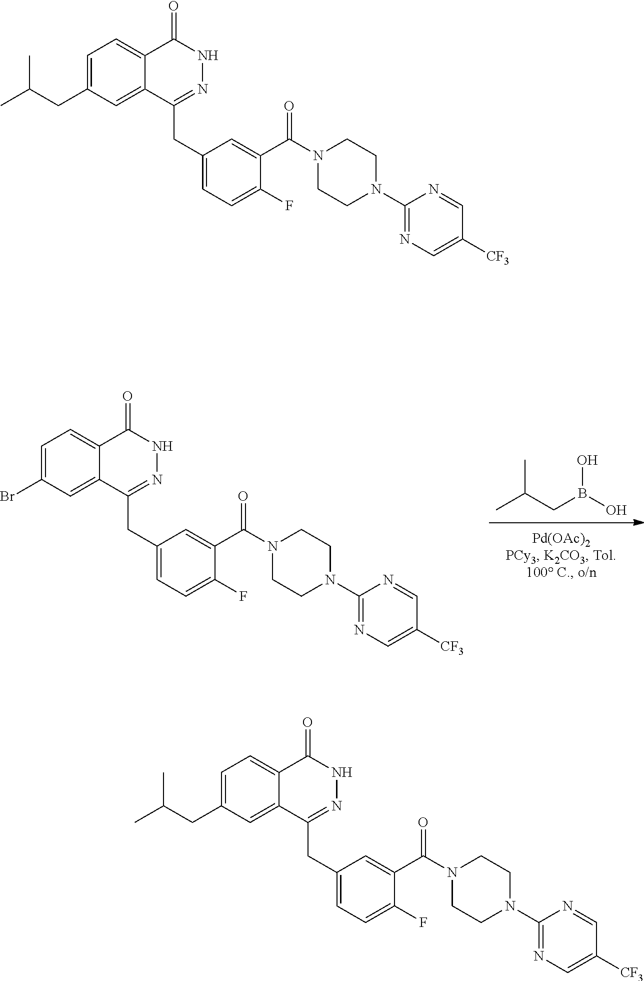

### Step 1

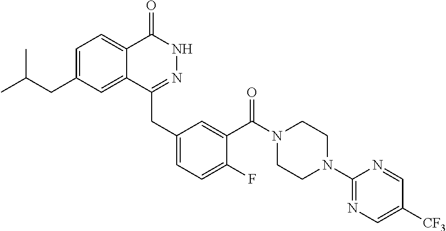 [SMILES: CC(C)Cc1ccc2c(=O)[nH]nc(Cc3ccc(F)c(C(=O)N4CCN(c5ncc(C(F)(F)F)cn5)CC4)c3)c2c1]

### 4-(4-Fluoro-3-(4-(5-(trifluoromethyl)pyrimidin-2-yl)piperazine-1-carbonyl)benzyl)-6-isobutylphthalazin-1(2H)-one

6-Bromo-4-(4-fluoro-3-(4-(5-(trifluoromethyl)pyrimidin-2-yl)piperazine-1-carbonyl)benzyl)phthalazin-1(2H)-one (0.030 g, 0.05 mmol) isobutylboronic acid (0.011 g, 3.0 equiv), Pd(OAc)₂ (2 mg), PCy₃ (4 mg), K₂CO₃ (21 mg), and toluene were added in a pressure vial under N₂ atmosphere. The mixture was heated at 100° C. overnight. The reaction was cooled to rt and concentrated in vacuo. The crude residue was purified by flash chromatography (0-100% EtOAc in hexanes) to afford the product as a white solid (12.8 mg, 49% yield). LCMS ESI m/z: 568.4 [M+H]⁺. ¹H NMR (400 MHz, CDCl₃) δ 10.25 (s, 1H), 8.43 (s, 2H), 8.31 (d, J=8.0 Hz, 1H), 7.48 (d, J=8.0 Hz, 1H), 7.38 (s, 1H), 7.28 (d, J=17.5 Hz, 2H), 6.98 (t, J=8.8 Hz, 1H), 4.22 (s, 2H), 3.94 (s, 2H), 3.84-3.77 (m, 4H), 3.31 (s, 2H), 2.54 (d, J=7.1 Hz, 2H), 1.80 (m, 1H), 0.79 (d, J=6.6 Hz, 6H).

### Synthesis of Example 45: 6-Cyclopropyl-4-(4-fluoro-3-(4-(5-(trifluoromethyl)pyrimidin-2-yl)piperazine-1-carbonyl)benzyl)phthalazin-1(2H)-one

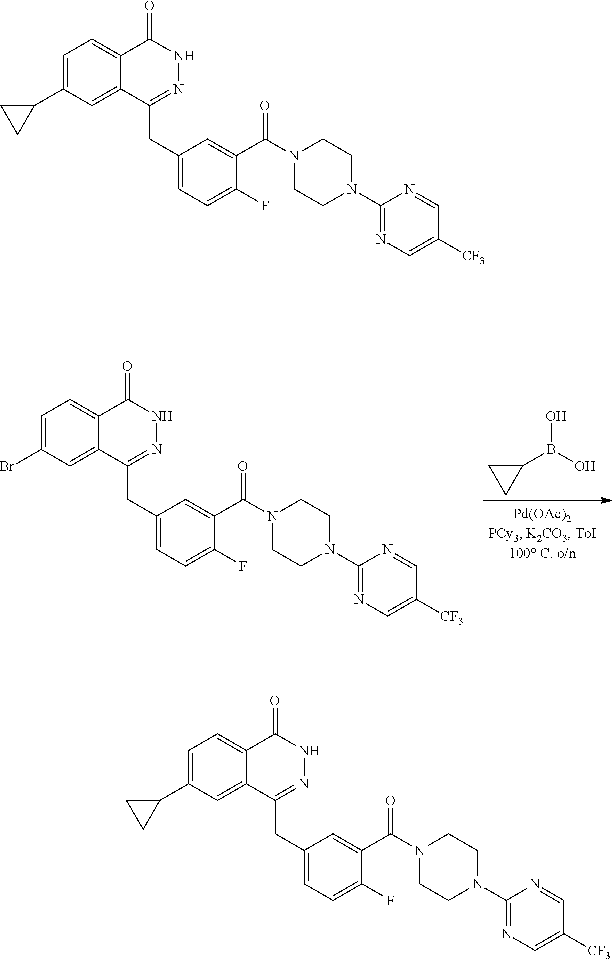 [SMILES: O=C(c1cc(Cc2n[nH]c(=O)c3ccc(C4CC4)cc23)ccc1F)N1CCN(c2ncc(C(F)(F)F)cn2)CC1 ; O=C(c1cc(Cc2n[nH]c(=O)c3ccc(Br)cc23)ccc1F)N1CCN(c2ncc(C(F)(F)F)cn2)CC1 ; OB(O)C1CC1 ; O=C(c1cc(Cc2n[nH]c(=O)c3ccc(C4CC4)cc23)ccc1F)N1CCN(c2ncc(C(F)(F)F)cn2)CC1]

### Step 1

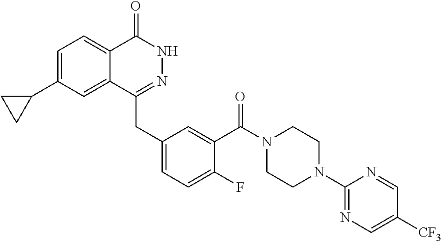 [SMILES: O=C(c1cc(Cc2n[nH]c(=O)c3ccc(C4CC4)cc23)ccc1F)N1CCN(c2ncc(C(F)(F)F)cn2)CC1]

### 6-Cyclopropyl-4-(4-fluoro-3-(4-(5-(trifluoromethyl)pyrimidin-2-yl)piperazine-1-carbonyl)benzyl)phthalazin-1(2H)-one

6-Bromo-4-(4-fluoro-3-(4-(5-(trifluoromethyl)pyrimidin-2-yl)piperazine-1-carbonyl)benzyl)phthalazin-1(2H)-one (0.030 g, 0.05 mmol), cyclopropylboronic acid (0.0065 g, 3.0 equiv), Pd(OAc)₂ (2 mg), PCy₃ (4 mg). K₂CO₃ (21 mg), and toluene were added in a pressure vial under N₂ atmosphere. The resulting mixture was heated at 100° C. overnight. The reaction was cooled to rt and concentrated in vacuo. The crude residue was purified via flash chromatography (0-100% EtOAc in hexanes) to afford the product as a white solid (9.8 mg, 37% yield). LCMS ESI m/z: 552.7 [M+H]⁺. ¹H NMR (400 MHz, CDCl₃) δ 10.12 (s, 1H), 8.43 (s, 2H), 8.26 (d, J=7.9 Hz, 1H), 7.34-7.27 (m, 4H), 7.00 (t, J=8.8 Hz, 1H), 4.19 (s, 2H), 3.92-3.77 (m, 4H), 3.32 (s, 2H), 1.94 (m, 1H), 0.87-0.65 (m, 4H).

### Synthesis of Example 46: 6-(3-(Dimethylamino)prop-1-yn-1-yl)-4-(4-fluoro-3-(4-(5-(trifluoromethyl)pyrimidin-2-yl)piperazine-1-carbonyl)benzyl)phthalazin-1(2H)-one

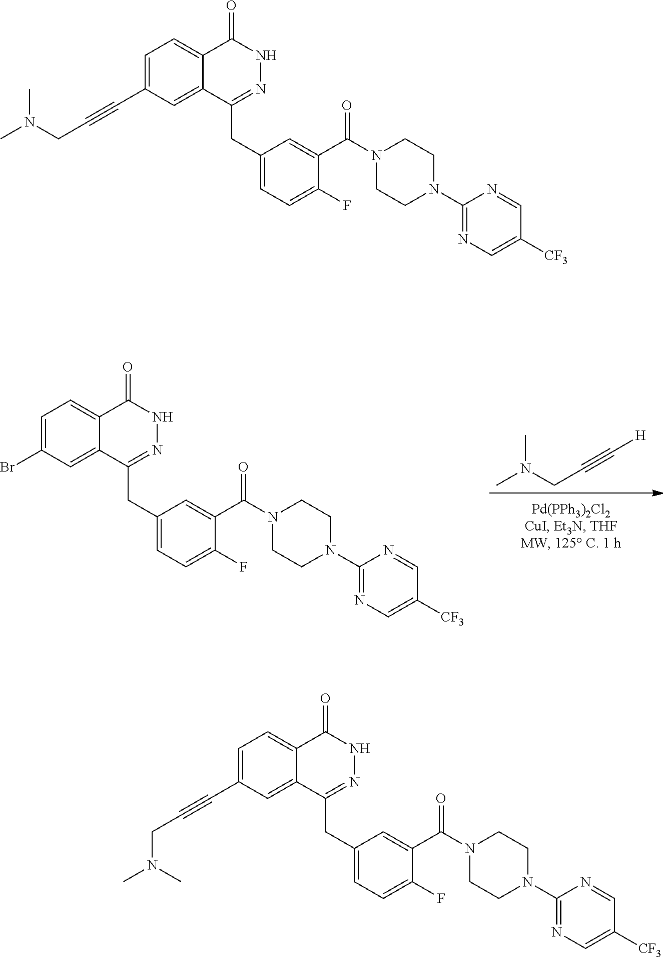

### Step 1

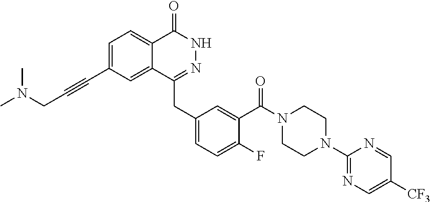 [SMILES: CN(C)CC#Cc1ccc2c(=O)[nH]nc(Cc3ccc(F)c(C(=O)N4CCN(c5ncc(C(F)(F)F)cn5)CC4)c3)c2c1]

### 6-(3-(Dimethylamino)prop-1-yn-1-yl)-4-(4-fluoro-3-(4-(5-(trifluoromethyl)pyrimidin-2-yl)piperazine-1-carbonyl)benzyl)phthalazin-1(2H)-one

6-bromo-4-(4-fluoro-3-(4-(5-(trifluoromethyl)pyrimidin-2-yl)piperazine-1-carbonyl)benzyl)phthalazin-1(2H)-one (0.030 g, 0.05 mmol) was added to a microwave vial and dissolved in anhydrous THF (1 mL) under argon. CuI (4 mg) and Pd(PPh₃)₂Cl₂ (2 mg) was added to the stirred solution under argon and the mixture was stirred at rt for 15 mins. To this mixture as added triethylamine (60 uL), stirred for another 5 mins, and N,N-dimethylprop-2-yn-1-amine was added dropwise. The mixture was heated in a microwave reactor at 125° C. for 1 hr. The crude residue was purified via a flash chromatography (0-80% EtOAc in hexanes) to afford the product as a white solid (9.0 mg, 30% yield). LCMS ESI m/z: 593.9 [M+H]⁺. ¹H NMR (400 MHz, CDCl₃) δ 10.46 (s, 1H), 8.44 (s, 2H), 8.33 (d, J=8.2 Hz, 1H), 7.82-7.67 (m, 2H), 7.28 (ddd, J=13.7, 5.6, 2.4 Hz, 2H), 7.01 (t, J=8.8 Hz, 1H), 4.21 (s, 2H), 3.95 (s, 2H), 3.86-3.79 (m, 4H), 3.64 (s, 2H), 3.33 (s, 2H), 2.50 (s, 6H).

### Synthesis of Example 47: 6-(Cyclopropylethynyl)-4-(4-fluoro-3-(4-(5-(trifluoromethyl)pyrimidin-2-yl)piperazine-1-carbonyl)benzyl)phthalazin-1(2)-one

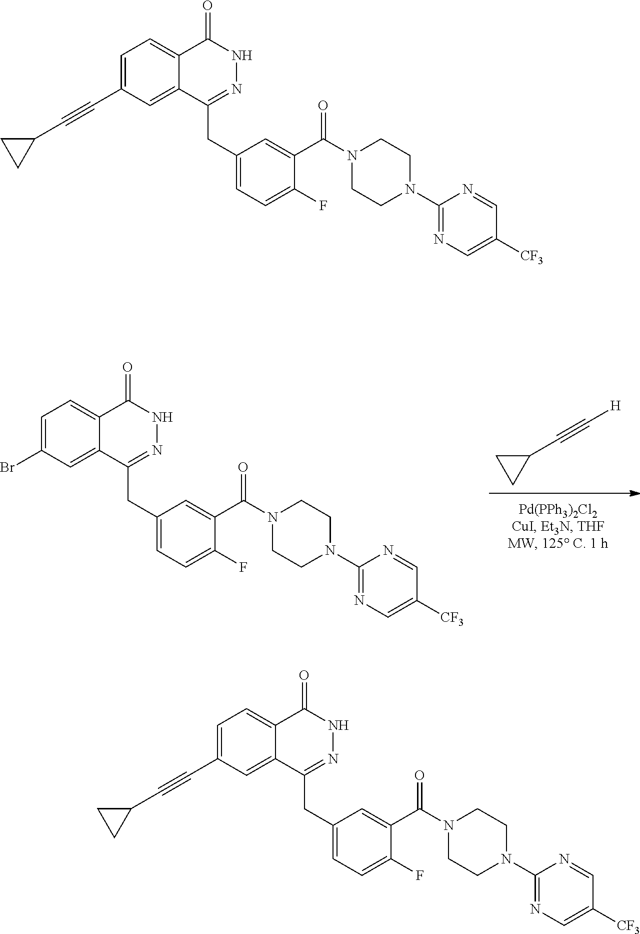 [SMILES: O=C(c1cc(Cc2n[nH]c(=O)c3ccc(C#CC4CC4)cc23)ccc1F)N1CCN(c2ncc(C(F)(F)F)cn2)CC1 ; O=C(c1cc(Cc2n[nH]c(=O)c3ccc(Br)cc23)ccc1F)N1CCN(c2ncc(C(F)(F)F)cn2)CC1 ; C#CC1CC1 ; O=C(c1cc(Cc2n[nH]c(=O)c3ccc(C#CC4CC4)cc23)ccc1F)N1CCN(c2ncc(C(F)(F)F)cn2)CC1]

### Step 1

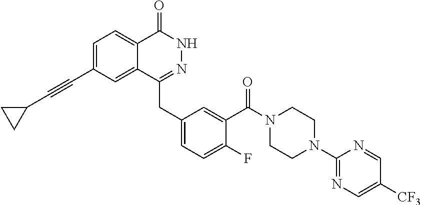 [SMILES: O=C(c1cc(Cc2n[nH]c(=O)c3ccc(C#CC4CC4)cc23)ccc1F)N1CCN(c2ncc(C(F)(F)F)cn2)CC1]

### 6-(Cyclopropylethynyl)-4-(4-fluoro-3-(4-(5-(trifluoromethyl)pyrimidin-2-yl)piperazine-1-carbonyl)benzyl)phthalazin-1(2H)-one

6-Bromo-4-(4-fluoro-3-(4-(5-(trifluoromethyl)pyrimidin-2-yl)piperazine-1-carbonyl)benzyl)phthalazin-1(2H)-one (0.030 g, 0.05 mmol) was added to a microwave vial and dissolved in anhydrous THF (1 mL) under argon gas. CuI (4 mg) and Pd(PPh₃)₂Cl₂ (2 mg) was added to the stirred solution under argon and the mixture was stirred at rt for 15 mins. To this mixture was added triethylamine (60 uL), stirred for another 5 mins, and then the cyclopropylalkyne was added dropwise. The mixture was heated in a microwave reactor at 125° C. for 1 hr. The crude residue was purified via a flash chromatography (0-80% EtOAc in hexanes) to afford the product as a white solid (0.014 g, 49% yield). LCMS ESI m/z: 576.9 [M+H]⁺. ¹H NMR (400 MHz, CDCl₃) δ 10.58 (s, 1H), 8.50 (s, 2H), 8.35 (d, J=8.8 Hz, 1H), 7.72-7.62 (m, 2H), 7.34-7.42 (m, 2H), 7.07 (t, J=8.8 Hz, 1H), 4.25 (s, 2H), 4.02 (s, 2H), 3.40 (s, 2H), 1.55-1.41 (m, 1H), 0.97-0.77 (m, 4H).

### Synthesis of Example 48: 4-(4-Fluoro-3-(4-(5-(trifluoromethyl)pyrimidin-2-yl)piperazine-1-carbonyl)benzyl)-6-(prop-1-yn-1-yl)phthalazin-1(2H)-one

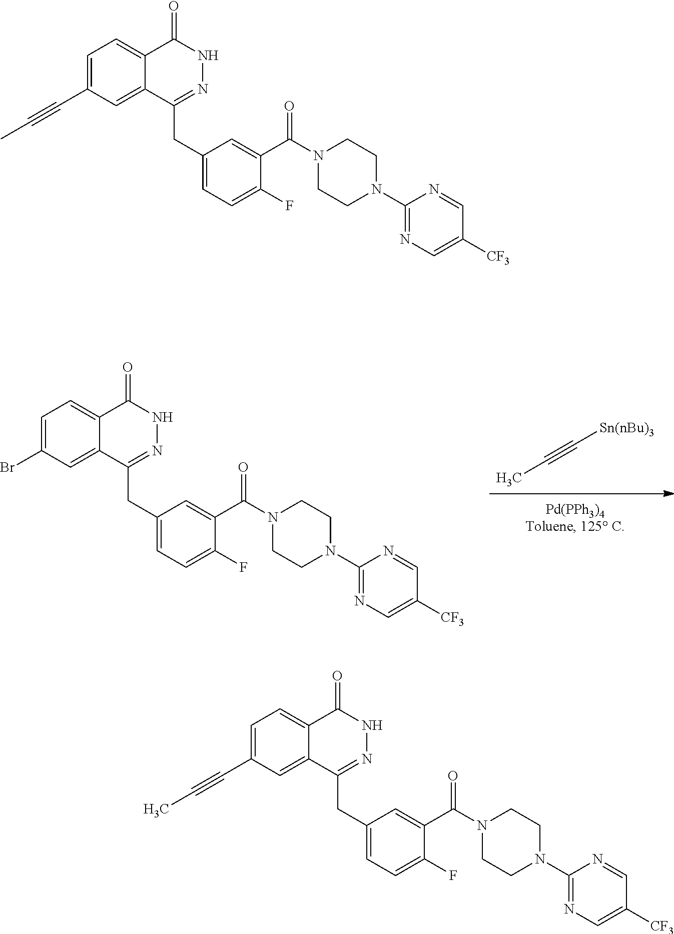

### Step 1:

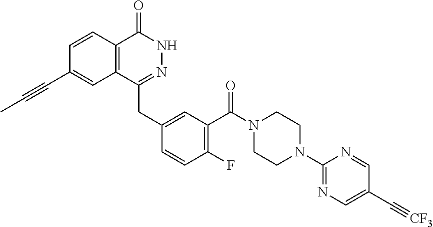

### 4-(4-Fluoro-3-(4-(5-(trifluoromethyl)pyrimidin-2-yl)piperazine-1-carbonyl)benzyl)-6-(prop-1-yn-1-yl)phthalazin-1(2H)-one

6-Bromo-4-(4-fluoro-3-(4-(5-(trifluoromethyl)pyrimidin-2-yl)piperazine-1-carbonyl)benzyl)phthalazin-1(2H)-one (0.029 g, 0.053 mmol) was dissolved in anhydrous toluene (1 mL) under argon gas and the solution degassed with argon for 5 min. Palladium tetrakis(triphenylphosphine) (7 mg, 0.00583 mmol) was added quickly to this mixture and the solution was degassed with argon for 10 min. Tributyl(prop-1-yn-1-yl)stannane (0.040 uL, 1.5 mmol) was added dropwise. The reaction mixture was heated at 125° C. for 4 h in a sealed tube. The reaction was cooled to rt and concentrated in vacuo. The crude residue was purified by flash chromatography (0-80% EtOAc in hexanes) to afford the product as a white solid (21.6 mg, 74% yield). LCMS ESI m/z: 550.2 [M+H]⁺. ¹H NMR (400 MHz, CD₃OD) δ 8.58-8.42 (m, 2H), 8.21 (d, J=8.3 Hz, 1H), 7.79 (s, 1H), 7.68 (dd, J=8.3, 1.4 Hz, 1H), 7.33 (dd, J=6.3, 2.2 Hz, 1H), 7.12 (t, J=9.0 Hz, 1H), 4.30 (s, 2H), 4.00-3.93 (m, 4H), 3.81-3.75 (m, 4H), 2.00 (s, 3H).

### Synthesis of Example 49: 4-(4-Fluoro-3-(4-(5-(trifluoromethyl)pyrimidin-2-yl)piperazine-1-carbonyl)benzyl)-6-(3,3,3-trifluoroprop-1-yn-1-yl)phthalazin-1(2H)-one

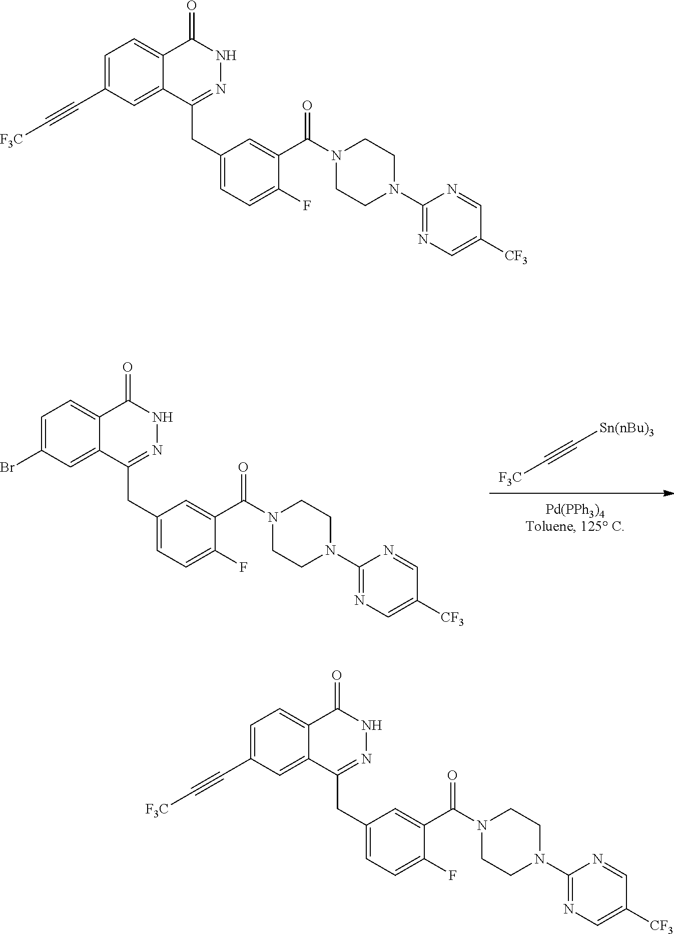

### Step 1

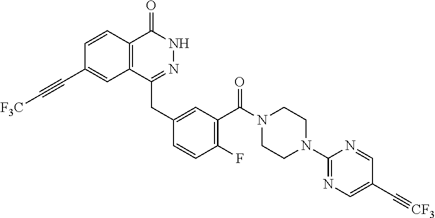

### 4-(4-Fluoro-3-(4-(5-(trifluoromethyl)pyrimidin-2-yl)piperazine-1-carbonyl)benzyl)-6-(3,3,3-trifluoroprop-1-yn-1-yl)phthalazin-1(2H)-one

6-Bromo-4-(4-fluoro-3-(4-(5-(trifluoromethyl)pyrimidin-2-yl)piperazine-1-carbonyl)benzyl)phthalazin-1(2H)-one (0.024 g, 0.04 mmol) was dissolved in anhydrous toluene (0.5 mL) under argon gas and the solution was degassed with argon for 5 min. Palladium tetrakis(triphenylphosphine) (7 mg, 0.00583 mmol) was added quickly and the solution degassed with argon and stirred for 10 min. To this mixture tributyl(3,3,3-trifluoroprop-1-yn-1-yl)stannane (0.025 uL, 0.06 mmol) was added dropwise. The reaction mixture was heated at 125° C. for 4 h in a sealed tube. The reaction mixture was cooled to rt and concentrated in vacuo. The crude residue was purified by flash chromatography (0-80% EtOAc in hexanes) to afford the product as a white solid (6.6 mg, 27% yield). LCMS ESI m/z: 605.0 [M+H]⁺. ¹H NMR (400 MHz, CDCl₃) 9.78 (s, 1H), 8.33 (s, 2H), 8.13 (d, J=8.9 Hz, 1H), 7.74-7.65 (m, 2H), 7.11-7.16 (m, 2H), 6.91 (t, J=8.8 Hz, 1H), 4.06 (s, 2H), 3.84 (s, 2H), 3.74-3.67 (m, 4f), 3.22 (s, 2H).

### Synthesis of Example 51: 6-(4-(5-((7-(3,3-Difluorocyclobutoxy)-4-oxo-3,4-dihydrophthalazin-1-yl)methyl)-2-fluorobenzoyl)piperazin-1-yl)nicotinonitrile

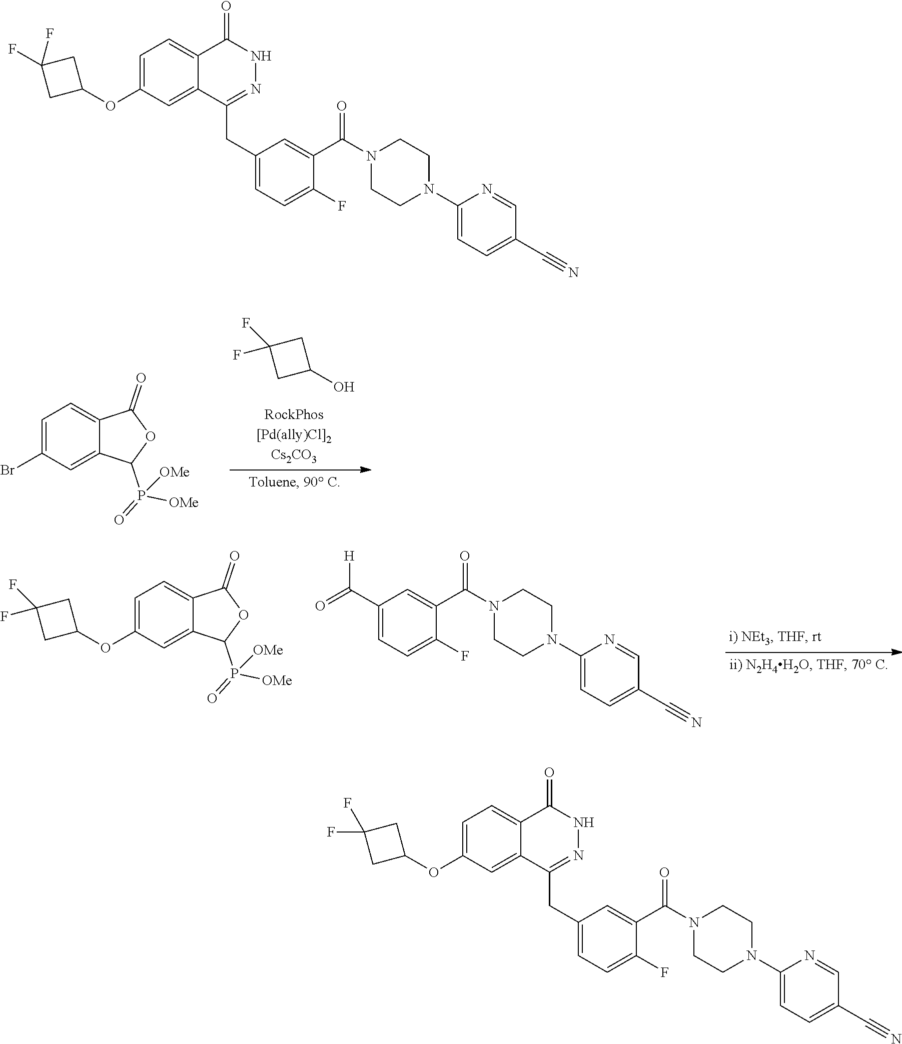 [SMILES: N#Cc1ccc(N2CCN(C(=O)c3cc(Cc4n[nH]c(=O)c5ccc(OC6CC(F)(F)C6)cc45)ccc3F)CC2)nc1 ; COP(=O)(OC)C1OC(=O)c2ccc(Br)cc21 ; OC1CC(F)(F)C1 ; COP(=O)(OC)C1OC(=O)c2ccc(OC3CC(F)(F)C3)cc21 ; N#Cc1ccc(N2CCN(C(=O)c3cc(C=O)ccc3F)CC2)nc1 ; N#Cc1ccc(N2CCN(C(=O)c3cc(Cc4n[nH]c(=O)c5ccc(OC6CC(F)(F)C6)cc45)ccc3F)CC2)nc1]

### Step 1

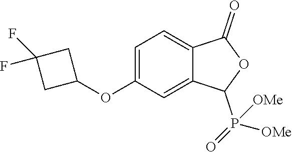 [SMILES: COP(=O)(OC)C1OC(=O)c2ccc(OC3CC(F)(F)C3)cc21]

### Dimethyl (6-(3,3-difluorocyclobutoxy)-3-oxo-1,3-dihydroisobenzofuran-1-yl)phosphonate

A mixture of dimethyl (6-bromo-3-oxo-1,3-dihydroisobenzofuran-1-yl)phosphonate (3H)-one (25 mg, 0.078 mmol), Pd₂(dba)₃ (2.5 mg, 0.007 mmol), Rockphos (6.1 mg, 0.012 mmol), cesium carbonate (38 mg, 0.12 mmol) and 3,3-difluorocyclobutanol (43 mg, 0.39 mmol) in toluene (1.7 mL) was stirred at 90° C. for 5 hours. The mixture was cooled to rt and concentrated in vacuo. The resulting dimethyl (6-(3,3-difluorocyclobutoxy)-3-oxo-1,3-dihydroisobenzofuran-1-yl)phosphonate was obtained as crude (brown oil) and carried forward without further purification. LCMS ESI m/z: 349.2 [M+H]⁺.

### Step 2

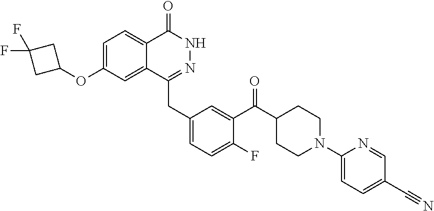 [SMILES: N#Cc1ccc(N2CCC(C(=O)c3cc(Cc4n[nH]c(=O)c5ccc(OC6CC(F)(F)C6)cc45)ccc3F)CC2)nc1]

### 6-(4-(5-((7-(3,3-Difluorocyclobutoxy)-4-oxo-3,4-dihydrophthalazin-1-yl)methyl)-2-fluorobenzoyl)piperazin-1-yl)nicotinonitrile

A mixture of dimethyl (6-(3,3-difluorocyclobutoxy)-3-oxo-1,3-dihydroisobenzofuran-1-yl)phosphonate (25 mg, 0.078 mmol), 6-(4-(2-fluoro-5-formylbenzoyl)piperazin-1-yl)nicotinonitrile (24 mg, 0.078 mmol) and Et₃N (22 mg, 0.21 mmol) in THF (0.7 mL) was stirred at rt for 17 hours. To the reaction mixture was added N₂H₄·H₂O (0.01 mL, 0.32 mmol), then stirred at 70° C. for 1 hour. The reaction mixture was concentrated in vacuo and the resulting crude was subjected to reverse phase column chromatography (MeCN: ammonium formate buffered water=0 to 100%) to afford 6-(4-(5-((7-(3,3-difluorocyclobutoxy)-4-oxo-3,4-dihydrophthalazin-1-yl)methyl)-2-fluorobenzoyl)piperazin-1-yl)nicotinonitrile as a white solid (4 mg, 0.059 mmol, 12% yield). LCMS ESI m/z: 575.3 [M+H]⁺. ¹H-NMR (400 MHz, DMSO-d₆) δ 8.51 (d, J=2.2 Hz, 1H), 8.19 (d, J=8.8 Hz, 1H), 7.90 (dd, J=9.2, 2.2 Hz, 1H), 7.47-7.42 (m, 2H), 7.39 (dd, J=8.8, 2.3 Hz, 1H), 7.28 (d, J=9.3 Hz, 1H), 7.18 (d, J=1.7 Hz, 1H), 6.92 (d, J=9.1 Hz, 1H), 4.99-4.91 (m, 1H), 4.32 (s, 2H), 3.78-3.74 (m, 2H), 3.73-3.69 (m, 2H), 3.63-3.59 (m, 2H), 3.33-3.28 (m, 2H), 3.26-3.18 (m, 2H), 2.76-2.67 (m, 2H).

### Synthesis of Example 52: 6-(4-(5-((7-(3-cyanocyclobutoxy)-4-oxo-3,4-dihydrophthalazin-1-yl)methyl)-2-fluorobenzoyl)piperazin-1-yl)nicotinonitrile formate salt

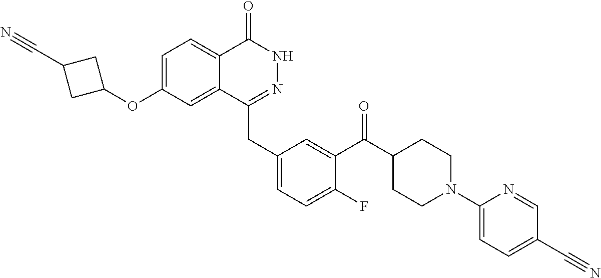 [SMILES: N#Cc1ccc(N2CCC(C(=O)c3cc(Cc4n[nH]c(=O)c5ccc(OC6CC(C#N)C6)cc45)ccc3F)CC2)nc1]

Following the procedure described in Example 51 and making non-critical variations as required to replace dimethyl (6-(3,3-difluorocyclobutoxy)-3-oxo-1,3-dihydroisobenzofuran-1-yl)phosphonate with dimethyl (6-(3-cyanocyclobutoxy)-3-oxo-1,3-dihydroisobenzofuran-1-yl)phosphonate, 6-(4-(5-((7-(3-cyanocyclobutoxy)-4-oxo-3,4-dihydrophthalazin-1-yl)methyl)-2-fluorobenzoyl)piperazin-1-yl)nicotinonitrile was obtained as a white solid (1 mg, 0.002 mmol, 2% yield). LCMS ESI m/z: 564.5 [M+H]⁺. ¹H NMR (400 MHz, DMSO-d₆) δ 12.50 (s, 1H), 8.51 (d, J=1.9 Hz, 1H), 8.48 (s, 1H), 8.17 (dd, J=8.9, 2.1 Hz, 1H), 7.90 (dd, J=9.1, 2.3 Hz, 1H), 7.45-7.41 (m, 2H), 7.33 (dd, J=8.9, 2.3 Hz, 1H), 7.27 (t, J=8.9 Hz, 1H), 7.12 (dd, J=7.0, 3.0 Hz, 1H), 6.93 (d, J=9.1 Hz, 1H), 4.90-4.82 (m, 1H), 4.30 (s, 2H), 3.79-3.76 (m, 2H), 3.74-3.71 (m, 2H), 3.62 (dd, J=6.0, 3.3 Hz, 2H), 3.17-3.08 (m, 3H), 2.96-2.84 (m, 2H), 2.40-2.33 (m, 2H).

### Synthesis of Example 53: 6-(4-(5-((7-(cyclobutylsulfonyl)-4-oxo-3,4-dihydrophthalazin-1-yl)methyl)-2-fluorobenzoyl)piperazin-1-yl)nicotinonitrile

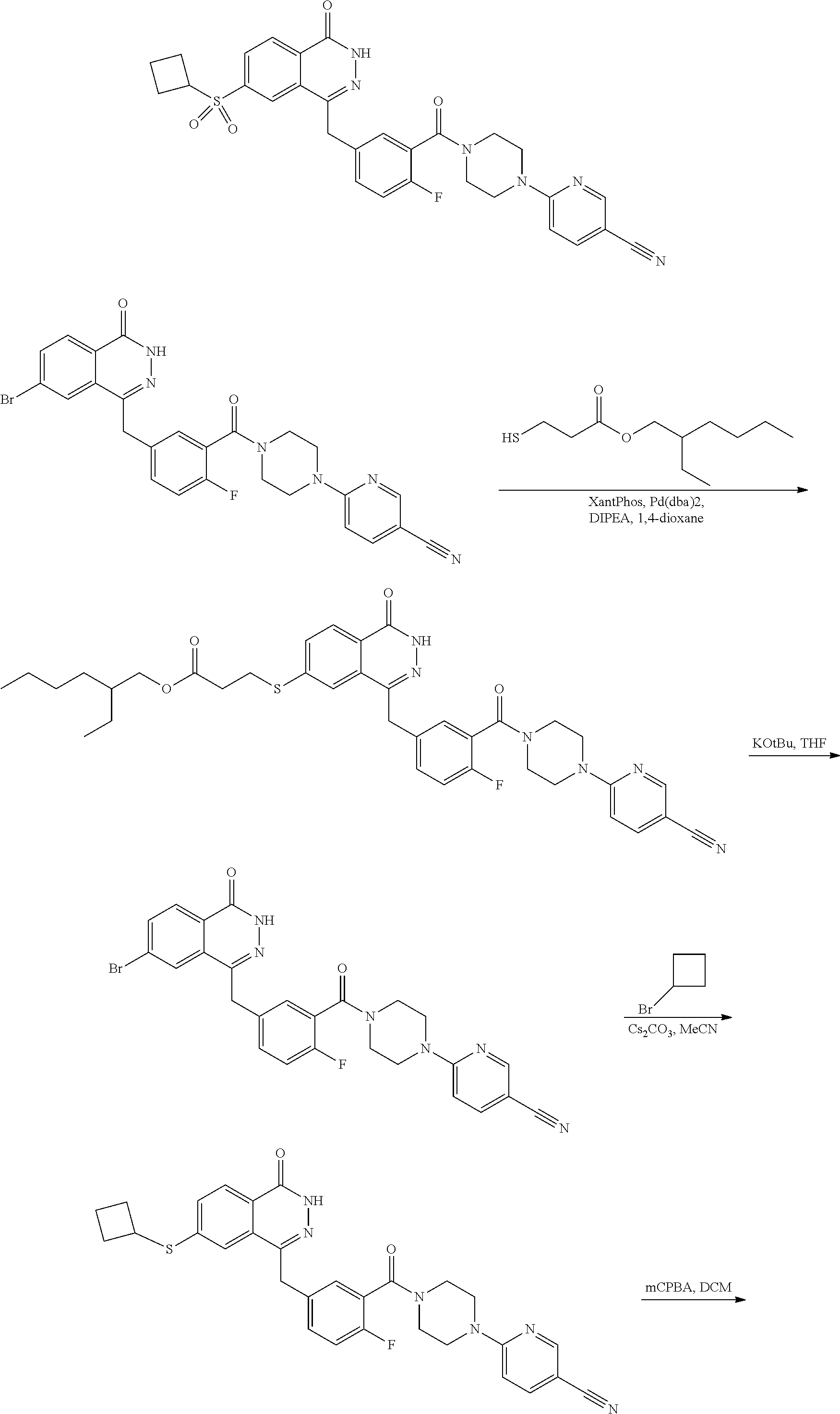

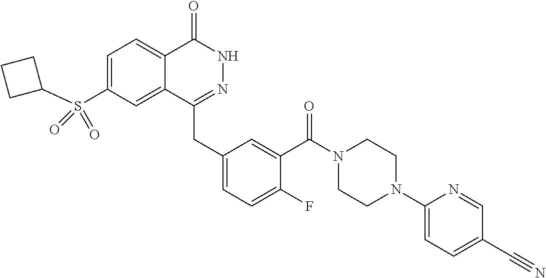 [SMILES: N#Cc1ccc(N2CCN(C(=O)c3cc(Cc4n[nH]c(=O)c5ccc(S(=O)(=O)C6CCC6)cc45)ccc3F)CC2)nc1]

### Step 1

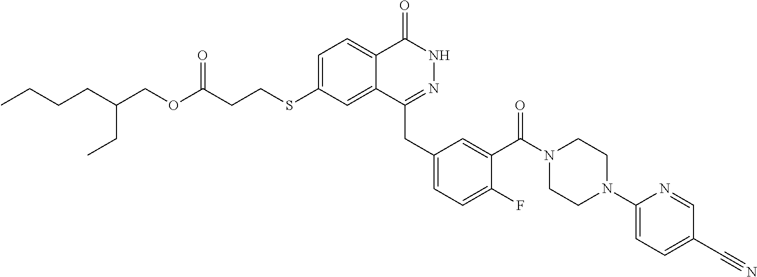 [SMILES: CCCCC(CC)COC(=O)CCSc1ccc2c(=O)[nH]nc(Cc3ccc(F)c(C(=O)N4CCN(c5ccc(C#N)cn5)CC4)c3)c2c1]

### 2-Ethylhexyl 3-((4-(3-(4-(5-cyanopyridin-2-yl)piperazine-1-carbonyl)-4-fluorobenzyl)-1-oxo-1,2-dihydrophthalazin-6-yl)thio)propanoate

To a solution of 6-(4-(5-((7-bromo-4-oxo-3,4-dihydrophthalazin-1-yl)methyl)-2-fluorobenzoyl)piperazin-1-yl)nicotinonitrile (200 mg, 0.365 mmol), 2-Ethylhexyl-3-Mercaptopropionate (80 mg, 0.365 mmol) and N,N-diisopropylethylamine (129 μL, 0.731 mmol) in 1,4-dioxane (3.65 mL) were added bis(dibenzylideneacetone)palladium(0) (4 mg, 0.007 mmol) and 4,4-bis(diphenylphosphino)-9,9-dimethylxanthene (8 mg, 0.014 mmol) at room temperature. The resulting mixture was degassed with N₂ for 5 mins charged with a N₂ balloon. The reaction mixture was heated to 110° C. After 180 mins, the reaction mixture diluted with EtOAc (50 mL) and washed with brine. The organic layer was dried over Na₂SO₄ and filtered. The solvent was removed to afford the crude product of 2-ethylhexyl 3-((4-(3-(4-(5-cyanopyridin-2-yl)piperazine-1-carbonyl)-4-fluorobenzyl)-1-oxo-1,2-dihydrophthalazin-6-yl)thio)propanoate as yellow oil (172 mg, 69% yield). LCMS ESI m/z: 685.5 [M+H]⁺.

### Step 2

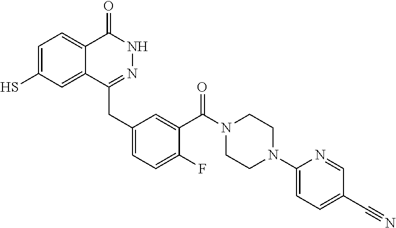 [SMILES: N#Cc1ccc(N2CCN(C(=O)c3cc(Cc4n[nH]c(=O)c5ccc(S)cc45)ccc3F)CC2)nc1]

### 6-(4-(2-Fluoro-5-((7-mercapto-4-oxo-3,4-dihydrophthalazin-1-yl)methyl)benzoyl)piperazin-1-yl)nicotinonitrile

To a solution of 2-ethylhexyl 3-((4-(3-(4-(5-cyanopyridin-2-yl)piperazine-1-carbonyl)-4-fluorobenzyl)-1-oxo-1,2-dihydrophthalazin-6-yl)thio)propanoate (142 mg, 0.207 mmol) in THF (2.07 mL) was added potassium tert-butoxide (58 uL, 0.415 mmol) at 0° C. After 110 mins, the reaction mixture was concentrated under reduced pressure and purified with C-18 silica gel reverse-phase chromatography using ACN/AmF from 0% to 60% to afford 6-(4-(2-fluoro-5-((7-mercapto-4-oxo-3,4-dihydrophthalazin-1-yl)methyl)benzoyl)piperazin-1-yl)nicotinonitrile (78.0 mg, 71% yield) as a white solid. LCMS ESI m/z: 501.3 [M+H]⁺.

### Step 3

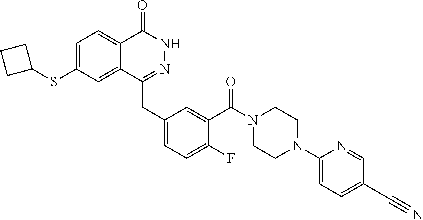 [SMILES: N#Cc1ccc(N2CCN(C(=O)c3cc(Cc4n[nH]c(=O)c5ccc(SC6CCC6)cc45)ccc3F)CC2)nc1]

### 6-(4-(5-((7-(cyclobutylthio)-4-oxo-3,4-dihydrophthalazin-1-yl)methyl)-2-fluorobenzoyl)piperazin-1-yl)nicotinonitrile

To a solution of 6-(4-(2-fluoro-5-((7-mercapto-4-oxo-3,4-dihydrophthalazin-1-yl)methyl)benzoyl)piperazin-1-yl)nicotinonitrile (20 mg, 0.400 mmol) in MeCN (400 μL) were added bromocyclobutane (23 uL, 0.240 mmol) and cesium carbonate (13 mg, 0.400 mmol), The reaction vessel was charged with N₂ and heated to 120° C. with a microwave reactor. After 20 mins, the reaction was concentrated under reduced pressure. The crude was used in the next step without further purification. LCMS ESI m/z: 555.3 [M+H]⁺.

### Step 4

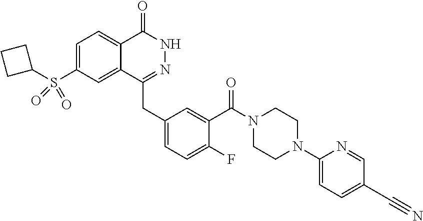 [SMILES: N#Cc1ccc(N2CCN(C(=O)c3cc(Cc4n[nH]c(=O)c5ccc(S(=O)(=O)C6CCC6)cc45)ccc3F)CC2)nc1]

### 6-(4-(5-((7-(cyclobutylsulfonyl)-4-oxo-3,4-dihydrophthalazin-1-yl)methyl)-2-fluorobenzoyl)piperazin-1-yl)nicotinonitrile

To a solution of the crude product of 6-(4-(5-((7-cyclobutylthio)-4-oxo-3,4-dihydrophthalazin-1-yl)methyl)-2-fluorobenzoyl)piperazin-1-yl)nicotinonitrile (25 mg, 0.451 mmol) in dichloromethane (2.25 mL) was added 3-chloroperbenzoic acid (31 mg, 0.180 mmol), The resulting suspension was stirred at room temperature. After 2 hours, the reaction mixture was concentrated under reduced pressure. The crude was purified with C18 silica gel reverse-phase chromatography using MeCN/Ammonium formate from 0% to 70% to afford (17 mg, 63% yield) as a white powder. LCMS ESI m/z: 587.3 [M+H]⁺. ¹H NMR (400 MHz, DMSO-d₆) δ 12.89 (s, 1H), 8.49 (d, J=2.1 Hz, 1H), 8.46 (d, J=8.2 Hz, 1H), 8.24-8.16 (m, 2H), 7.89 (dd, J=9.1, 2.3 Hz, 1H), 7.44-7.34 (m, 2H), 7.25 (t, J=9.0 Hz, 1H), 6.89 (d, J=9.1 Hz, 1H), 4.43 (s, 2H), 4.21 (p, =8.0 Hz, 1H), 3.72 (dd, J=18.3, 4.1 Hz, 4H), 3.57 (s, 2H), 3.27 (d, J=4.4 Hz, 2H), 2.30-2.16 (m, 2H), 2.08-1.96 (m, 2H), 1.95-1.72 (m, 2H).

### Synthesis of Example 54: 6-(4-(5-((7-((3,3-Difluorocyclobutyl)sulfonyl)-4-oxo-3,4-dihydrophthalazin-1-yl)methyl)-2-fluorobenzoyl)piperazin-1-yl)nicotinonitrile

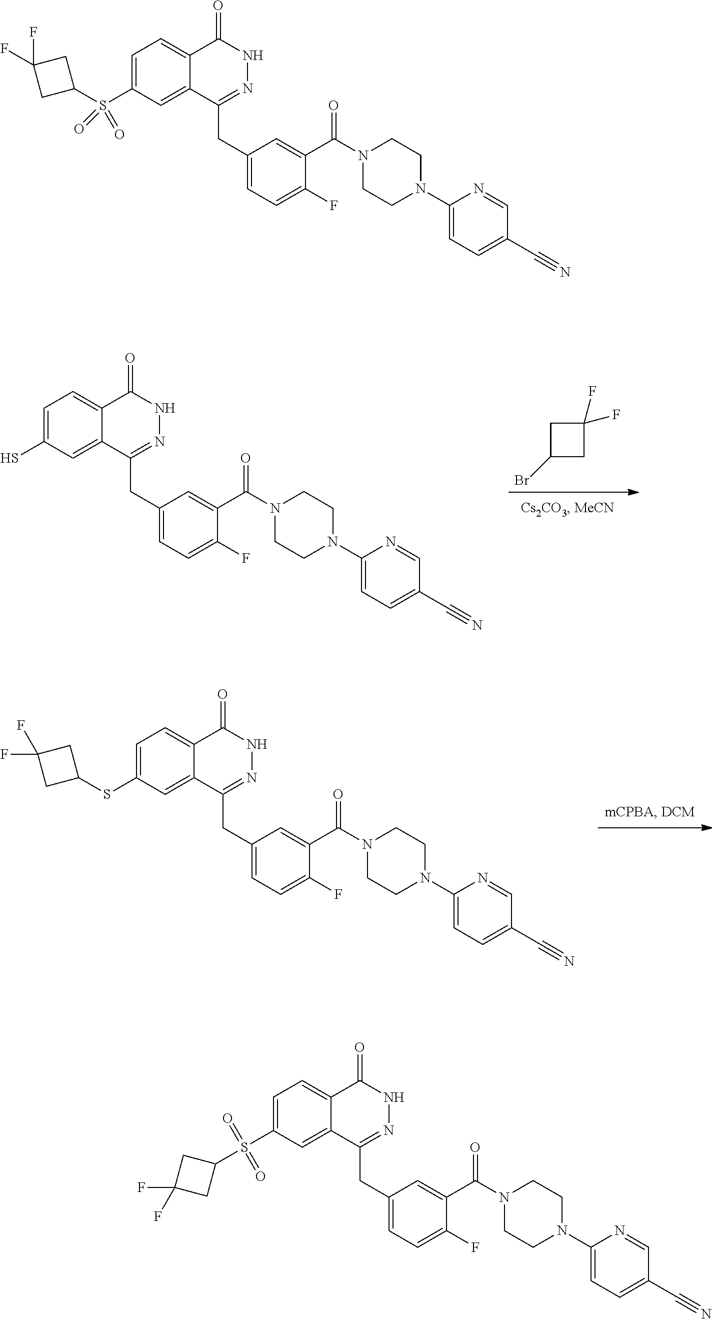 [SMILES: N#Cc1ccc(N2CCN(C(=O)c3cc(Cc4n[nH]c(=O)c5ccc(S(=O)(=O)C6CC(F)(F)C6)cc45)ccc3F)CC2)nc1 ; N#Cc1ccc(N2CCN(C(=O)c3cc(Cc4n[nH]c(=O)c5ccc(S)cc45)ccc3F)CC2)nc1 ; FC1(F)CC(Br)C1 ; N#Cc1ccc(N2CCN(C(=O)c3cc(Cc4n[nH]c(=O)c5ccc(SC6CC(F)(F)C6)cc45)ccc3F)CC2)nc1 ; N#Cc1ccc(N2CCN(C(=O)c3cc(Cc4n[nH]c(=O)c5ccc(S(=O)(=O)C6CC(F)(F)C6)cc45)ccc3F)CC2)nc1]

### Step 1

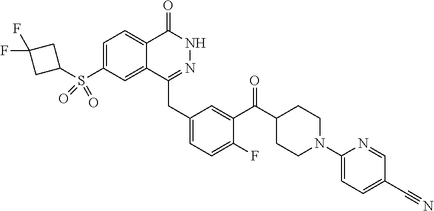 [SMILES: N#Cc1ccc(N2CCC(C(=O)c3cc(Cc4n[nH]c(=O)c5ccc(S(=O)(=O)C6CC(F)(F)C6)cc45)ccc3F)CC2)nc1]

### 6-(4-(5-((7-((3,3-difluorocyclobutyl)thio)-4-oxo-3,4-dihydrophthalazin-1-yl)methyl)-2-fluorobenzoyl)piperazin-1-yl)nicotinonitrile

Following the procedure described in Example 53 and making non-critical variation as required to replace bromocyclobutane with 3-bromo-1,1-difluorocyclobutane, the crude product of 6-(4-(5-((7-((3,3-difluorocyclobutyl)thio)-4-oxo-3,4-dihydrophthalazin-1-yl)methyl)-2-fluorobenzoyl)piperazin-1-yl)nicotinonitrile was obtained as a yellow oil (crude). LCMS ESI m/z: 591.3 [M+H]⁺.

### Step 2

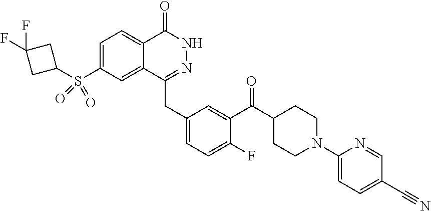 [SMILES: N#Cc1ccc(N2CCC(C(=O)c3cc(Cc4n[nH]c(=O)c5ccc(S(=O)(=O)C6CC(F)(F)C6)cc45)ccc3F)CC2)nc1]

### 6-(4-(5-((7-((3,3-difluorocyclobutyl)sulfonyl)-4-oxo-3,4-dihydrophthalazin-1-yl)methyl)-2-fluorobenzoyl)piperazin-1-yl)nicotinonitrile

Following the procedure described in Example 53 and making non-critical variation as required to replace 6-(4-(5-((7-(cyclobutylthio)-4-oxo-3,4-dihydrophthalazin-1-yl)methyl)-2-fluorobenzoyl)piperazin-1-yl)nicotinonitrile with 6-(4-(5-((7-(3,3-difluorocyclobutyl)thio)-4-oxo-3,4-dihydrophthalazin-1-yl)methyl)-2-fluorobenzoyl)piperazin-1-yl)nicotinonitrile, 6-(4-(5-((7-((3,3-difluorocyclobutyl)thio)-4-oxo-3,4-dihydrophthalazin-1-yl)methyl)-2-fluorobenzoyl)piperazin-1-yl)nicotinonitrile was obtained as a white solid (16 mg, 22% yield). LCMS ESI m/z: 623.4 [M+H]⁺. ¹H NMR (400 MHz, DMSO-d₆) δ 12.91 (s, 1H), 8.50-8.47 (m, 2H), 8.30 (d, J=1.2 Hz, 1H), 8.27 (dd, J=8.3, 1.6 Hz, 1H), 7.89 (dd, J=9.1, 2.3 Hz, 1H), 7.46-7.39 (m, 1H), 7.36 (dd, J=6.5, 2.1 Hz, 1H), 7.26 (t, J=9.0 Hz, 1H), 6.89 (d, J=9.1 Hz, 1H), 4.45 (s, 2H), 4.38-4.21 (m, 1H), 3.71 (dd, J=18.0, 4.1 Hz, 4H), 3.57 (s, 2H), 3.30 (d, J=20.9 Hz, 2H), 3.04-2.76 (m, 4H).

### Synthesis of Example 55: (SP-PAR7000203-NX-1)

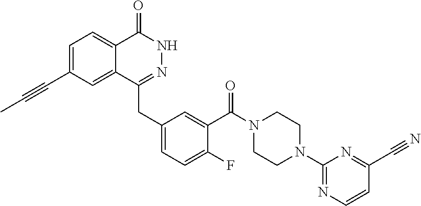 [SMILES: CC#Cc1ccc2c(=O)[nH]nc(Cc3ccc(F)c(C(=O)N4CCN(c5nccc(C#N)n5)CC4)c3)c2c1]

### 6-(4-(2-Fluoro-5-((4-oxo-7-(prop-1-ynyl)-3,4-dihydrophthalazin-1-yl)methyl)benzoyl)piperazin-1-yl)pyrazine-2-carbonitrile

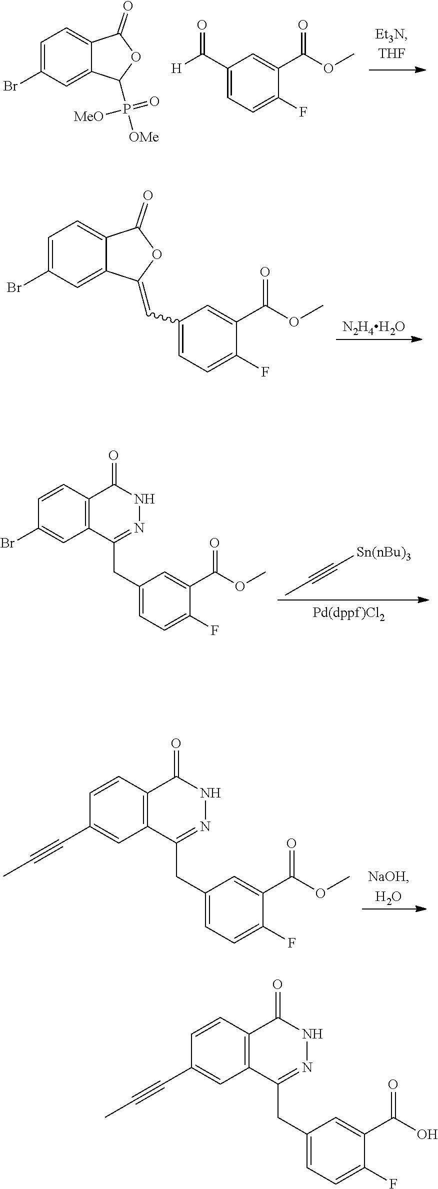

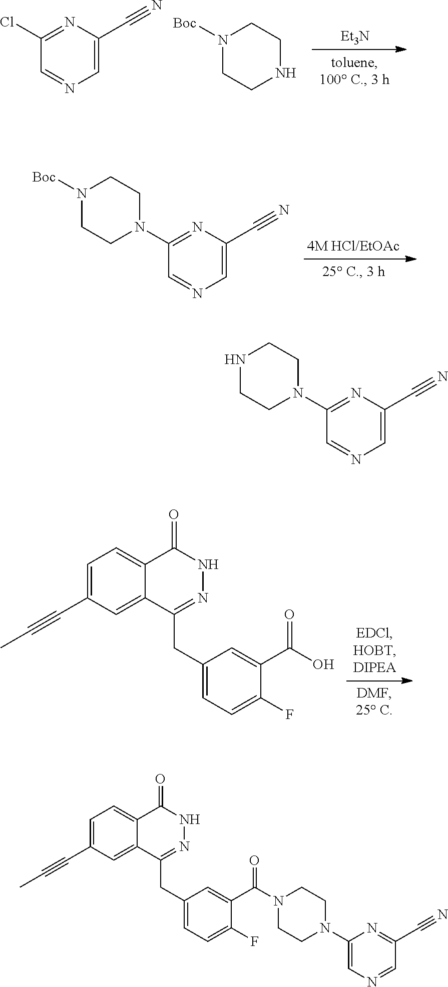

### Step 1

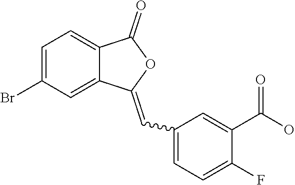 [SMILES: O=C(O)c1cc(C=C2OC(=O)c3ccc(Br)cc32)ccc1F]

### Methyl-5-((6-bromo-3-oxoisobenzofuran-1(3H)-ylidene)methyl)-2-fluorobenzoate

A solution of dimethyl (6-bromo-3-oxo-1,3-dihydroisobenzofuran-1-yl)phosphonate (5.0) g, 15.6 mmol), methyl 2-fluoro-5-formylbenzoate (3.12 g, 17.1 mmol) and Et₃N (4.73 g, 46.72 mmol) in THF (50 mL) was stirred at rt under Ar (g) for 12 hours. The reaction mixture was diluted with water (50 mL) and extracted with EtOAc (50 mL×3). The combined organic layer was washed with brine, dried over Na₂SO₄, filtered and concentrated in vacuo to afford methyl 5-((6-bromo-3-oxoisobenzofuran-1(3H)-ylidene)methyl)-2-fluorobenzoate as a white solid (5.01 g, 85% yield). This was used without further purification. LCMS ESI m/z: 377 (M+H)⁺.

### Step 2

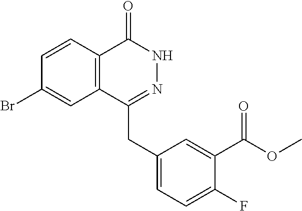 [SMILES: COC(=O)c1cc(Cc2n[nH]c(=O)c3ccc(Br)cc23)ccc1F]

### Methyl 5-((7-bromo-4-oxo-3,4-dihydrophthalazin-1-yl)methyl)-2-fluorobenzoate

A solution of methyl 5-((6-bromo-3-oxoisobenzofuran-1(3H)-ylidene)methyl)-2-fluorobenzoate (5.80 g, 13.3 mmol) and N₂H₄·H₂O (1.33 g, 26.5 mmol) in THF (50 mL) was stirred at 70° C. for 4 h. The solids were filtered and washed with water (30 mL) to afford methyl 5-((7-bromo-4-oxo-3,4-dihydrophthalazin-1-yl)methyl)-2-fluorobenzoate as a white solid (4.1 g, 79% yield). This was used without further purification. LCMS ESI m/z: 391 (M+H)⁺.

### Step 3

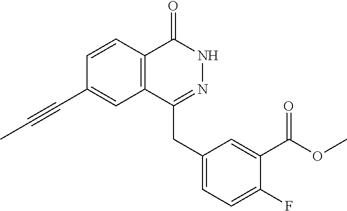 [SMILES: CC#Cc1ccc2c(=O)[nH]nc(Cc3ccc(F)c(C(=O)OC)c3)c2c1]

### Methyl 2-fluoro-5-((4-oxo-7-(prop-1-yn-1-yl)-3,4-dihydrophthalazin-1-yl)methyl)benzoate

A mixture of tributyl(prop-1-ynyl)stannane (8.08 g, 24.5 mmol), methyl 5-[(7-bromo-4-oxo-3H-phthalazin-1-yl)methyl]-2-fluoro-benzoate (4.00 g, 8.18 mmol) and Pd(dppf)Cl₂ (599 mg, 0.82 mmol) in 1,4-dioxane (40 mL) was stirred at 100° C. under Ar (g) for 5 hours. The reaction mixture was concentrated in vacuo and the residue was purified by silica gel chromatography (MeOH/DCM=0 to 20%) to afford methyl 2-fluoro-5-((4-oxo-7-prop-1-yn-1-yl)-3,4-dihydrophthalazin-1-yl)methyl)benzoate as a white solid (3.2 g, 96% yield). LCMS ESI m/z: 351 [M+H]⁺.

### Step 4

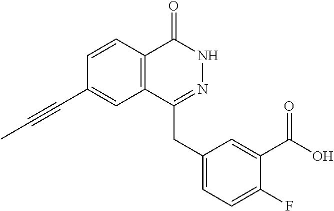 [SMILES: CC#Cc1ccc2c(=O)[nH]nc(Cc3ccc(F)c(C(=O)O)c3)c2c1]

### 2-Fluoro-5-((4-oxo-7-(prop-1-yn-1-yl)-3,4-dihydrophthalazin-1-yl)methyl)benzoic acid

A mixture of methyl 2-fluoro-5-((4-oxo-7-(prop-1-yn-1-yl)-3,4-dihydrophthalazin-1-yl)methyl)benzoate (3.20 g, 9.13 mmol) and NaOH (1.10 g, 27.40 mmol) in MeOH (30 mL) and H₂O (10 mL) was stirred at rt for 2 hours. The organic solvent was removed in vacuo and aq HCl (1N) was added until the solution reached pH 5˜6. The solids were collected by filtration, washed with H₂O (5 mL) and dried under vacuum to give 2-fluoro-5-((4-oxo-7-(prop-1-yn-1-yl)-3,4-dihydrophthalazin-1-yl)methyl)benzoic acid as a white solid (3.08 g, 90% yield). LCMS ESI m/z: 337 [M+H]⁺.

### Step 5

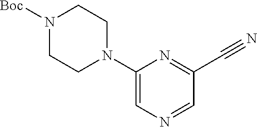 [SMILES: CC(C)(C)OC(=O)N1CCN(c2cncc(C#N)n2)CC1]

### tert-Butyl 4-(6-cyanopyrazin-2-yl)piperazine-1-carboxylate

To a solution of tert-butyl piperazine-1-carboxylate (265 mg, 1.07 mmol) in toluene (6 mL) was added 6-chloropyrazine-2-carbonitrile (150 mg, 1.07 mmol), triethylamine (326 mg, 3.22 mmol, 449 μL) at 25° C. After stirring the reaction mixture at 100° C. for 3 hr. Water and EtOAc were added to the mixture, which became two phases. The organic layer was separated, washed with NaCl (aq), dried over Na₂SO₄ and concentrated to give a residue. The residue was purified by silica gel chromatography (petroleum ether/EtOAc=20/1 to 2/1) to give tert-butyl 4-(6-cyanopyrazin-2-yl)piperazine-1-carboxylate (200 mg, crude) as a yellow solid. LCMS ESI m/z: 290.1 [M+H]⁺.

### Step 6 and 7

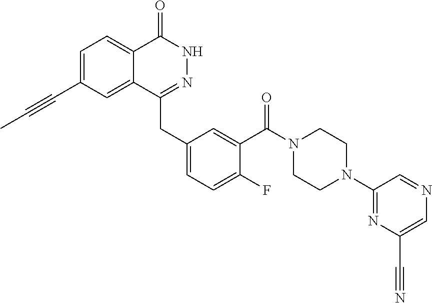 [SMILES: CC#Cc1ccc2c(=O)[nH]nc(Cc3ccc(F)c(C(=O)N4CCN(c5cncc(C#N)n5)CC4)c3)c2c1]

### 6-(4-(2-fluoro-5-((4-oxo-7-(prop-1-ynyl)-3,4-dihydrophthalazin-1-yl)methyl)benzoyl)piperazin-1-yl)pyrazine-2-carbonitrile

Following the two-step procedure in Example 59, but starting with tert-butyl 4-(6-cyanopyrazin-2-yl)piperazine-1-carboxylate gave the title compound as a white solid. LCMS ESI m/z: 508.2 [M+H]⁺. ¹H NMR (400 MHz, DMSO-d6) δ 12.62 (s, 1H), 8.64 (s, 1H), 8.35 (s, 1H), 8.20 (d, J=8.2 Hz, 1H), 7.93 (s, 1H), 7.78 (dd, J=8.2, 1.3 Hz, 1H), 7.45-7.38 (m, 2H), 7.29-7.23 (m, 1H), 4.34 (s, 2H), 3.75 (s, 4H), 3.59 (s, 2H), 3.34 (s, 2H), 2.09 (s, 3H).

### Synthesis of Example 56: 2-(4-(2-Fluoro-5-((4-oxo-7-(prop-1-ynyl)-3,4-dihydrophthalazin-1-yl)methyl)benzoyl)piperazin-1-yl)-N-methylisonicotinamide

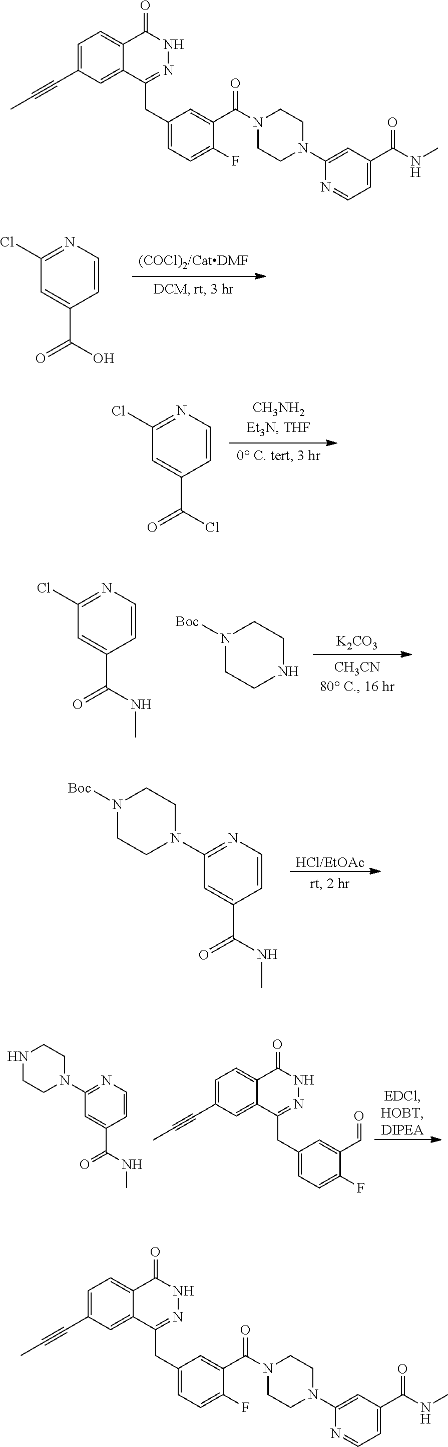

### Step 1, 2, 3, 4 and 5 were as in Example 55

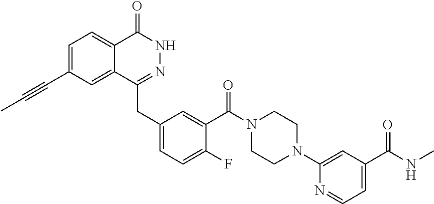 [SMILES: CC#Cc1ccc2c(=O)[nH]nc(Cc3ccc(F)c(C(=O)N4CCN(c5cc(C(=O)NC)ccn5)CC4)c3)c2c1]

### 2-(4-(2-Fluoro-5-((4-oxo-7-(prop-1-ynyl)-3,4-dihydrophthalazin-1-yl)methyl)benzoyl)piperazin-1-yl)-N-methylisonicotinamide

Following the five-step procedure above in Example 55, but starting with 2-chloroisonicotinic acid gave the title compound as a white solid. LCMS ESI m/z: 539.2 [M+H]⁺. ¹H NMR (400 MHz, DMSO-d6) δ 12.63 (s, 1H), 8.55 (d, J=4.6 Hz, 1H), 8.21 (dd, J=7.7, 7.0 Hz, 2H), 7.93 (s, 1H), 7.78 (dd, J=8.2, 1.3 Hz, 1H), 7.41 (dd, J=6.2, 2.2 Hz, 2H), 7.25 (t, J=9.3 Hz, 1H), 7.16 (s, 1H), 7.01 (d, J=5.2 Hz, 1H), 4.34 (s, 2H), 3.75 (s, 2H), 3.63 (s, 2H), 3.50 (s, 2H), 3.32 (s, 2H), 2.78 (d, J=4.5 Hz, 3H), 2.08 (s, 3H).

### Synthesis of Example 57: 4-(4-Fluoro-3-(4-(4,4,4-trifluorobutanoyl)piperazine-1-carbonyl)benzyl)-6-(prop-1-ynyl)phthalazin-1(2H)-one

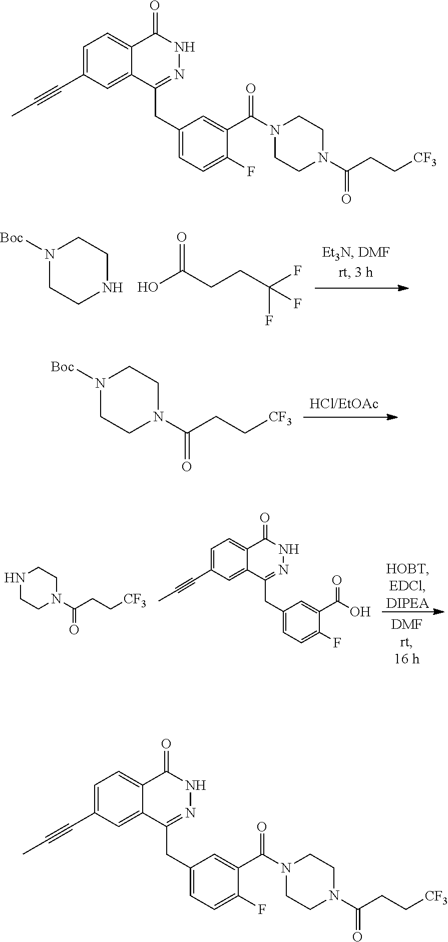

### Step 1

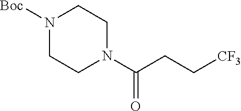 [SMILES: CC(C)(C)OC(=O)N1CCN(C(=O)CCC(F)(F)F)CC1]

### tert-Butyl 4-(4,4,4-trifluorobutanoyl)piperazine-1-carboxylate

To a solution of 4,4,4-trifluorobutanoic acid (577 mg, 4.06 mmol) in DMF (10 mL) was added Et₃N (1.64 g, 16.2 mmol) and HATU (2.32 g, 6.09 mmol), The reaction mixture was stirred at rt for 10 minutes, tert-butyl piperazine-1-carboxylate (1.0 g, 4.06 mmol) was added, and then stirred at rt for 3 hrs. The reaction mixture was poured into water (30 mL), extracted with Ethyl Acetate (50 mL×3), washed with brine, dried and concentrated. The residue was purified by silica gel chromatography to give tert-butyl 4-(4,4,4-trifluorobutanoyl)piperazine-1-carboxylate (750 mg, 59% yield) as a colorless oil. ¹H NMR (400 MHz, CDCl₃) δ 3.65-3.56 (m, 2H), 3.53-3.38 (m, 6H), 2.56 (m, 4H), 1.48 (s, 9H).

### Step 2 and 3

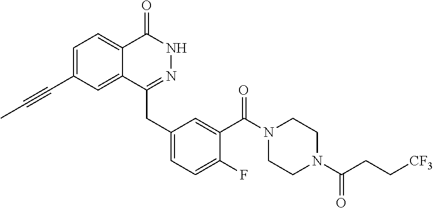 [SMILES: CC#Cc1ccc2c(=O)[nH]nc(Cc3ccc(F)c(C(=O)N4CCN(C(=O)CCC(F)(F)F)CC4)c3)c2c1]

### 4-(4-Fluoro-3-(4-(4,4,4-trifluorobutanoyl)piperazine-1-carbonyl)benzyl)-6-(prop-1-ynyl)phthalazin-1(2H)-one

Following the two-step procedure Example 59, but starting with tert-butyl 4-(4,4,4-trifluorobutanoyl)piperazine-1-carboxylate gave the title compound as a white solid. LCMS ESI m/z: 528.7 [M+H]⁺. ¹H NMR (400 MHz, DMSO-d6) δ 12.62 (s, 1H), 8.20 (d, J=8.3 Hz, 1H), 7.92 (d, J=13.2 Hz, 1H), 7.77 (d, J=7.3 Hz, 1H), 7.46-7.34 (m, 2H), 7.25 (d, J=7.2 Hz, 1H), 4.33 (s, 2H), 3.74-3.46 (m, 5H), 3.40 (m, 2H), 3.20 (m, 2H), 2.60 (m, 3H), 2.10 (s, 3H).

### Synthesis of Example 58: 3-(4-(2-Fluoro-5-((4-oxo-7-(prop-1-ynyl)-3,4-dihydrophthalazin-1-yl)methyl)benzoyl)piperazin-1-yl)-3-oxopropanenitrile

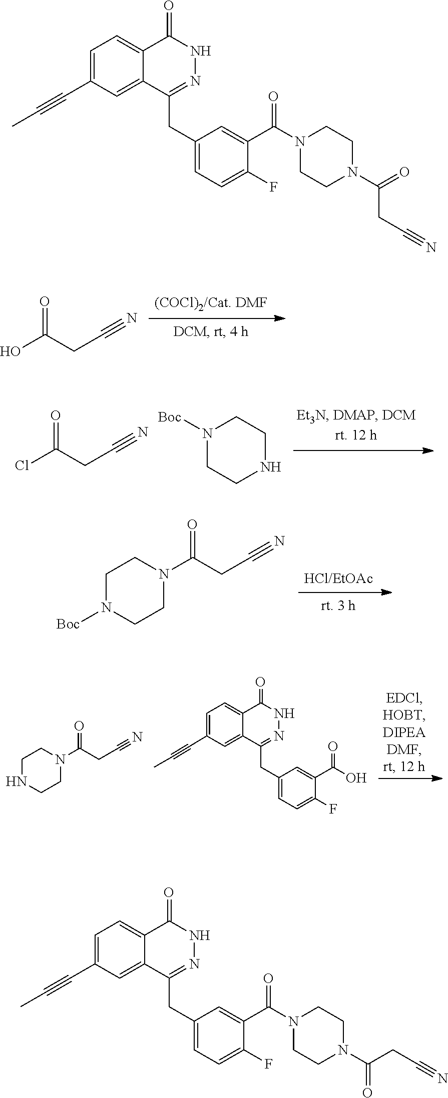

### Step 1

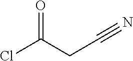

### 2-Cyanoacetyl chloride

Oxalyl dichloride (2.24 g, 17.6 mmol, 1.54 mL) was added drop-wise to a solution of 2-cyanoacetic acid (1.0) g, 11.8 mmol) in DCM (15 mL) and DMF (0.1 mL) at 0° C. The resultant mixture was stirred at rt for 4 hr. The mixture concentrated under vacuum to give 2-cyanoacetyl chloride (1.2 g, crude) as a black oil, which was used to next step without further purification.

### Step 2

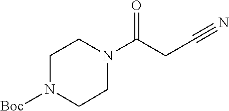 [SMILES: CC(C)(C)OC(=O)N1CCN(C(=O)CC#N)CC1]

### tert-Butyl 4-(2-cyanoacetyl)piperazine-1-carboxylate

2-Cyanoacetyl chloride (1.22 g, 11.79 mmol) in DCM (10 mL) was added to a solution of tert-butyl piperazine-1-carboxylate (3.19 g, 12.97 mmol), Et₃N (5.96 g, 58.93 mmol, 8.21 mL), DMAP (143.99 mg, 1.18 mmol) and DCM (10 mL) at 0° C. The resultant mixture was stirred at rt for 12 hr. The mixture was concentrated to give a crude residue, which was purified by silica gel chromatography (petroleum ether:EtOAc=10:1 to 1:1) to give tert-butyl 4-(2-cyanoacetyl)piperazine-1-carboxylate (1.00 g, 34% yield) as a yellow solid. ¹H NMR (400 MHz, CDCl₃) δ 3.61-3.54 (m, 2H), 3.53-3.46 (m, 4H), 3.41 (dd, J=9.9, 4.8 Hz, 4H), 1.44 (s, 9H).

### Step 3 and 4

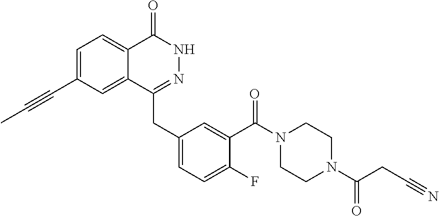 [SMILES: CC#Cc1ccc2c(=O)[nH]nc(Cc3ccc(F)c(C(=O)N4CCN(C(=O)CC#N)CC4)c3)c2c1]

### 3-(4-(2-Fluoro-5-((4-oxo-7-(prop-1-ynyl)-3,4-dihydrophthalazin-1-yl)methyl)benzoyl) piperazin-1-yl)-3-oxopropanenitrile

Following the two-step procedure in Example 59, but starting with tert-butyl 4-(2-cyanoacetyl)piperazine-1-carboxylate gave the title compound as a light yellow solid. LCMS ESI m/z: 472.2 [M+H]⁺. ¹H NMR (400 MHz, DMSO-d6) δ 12.62 (s, 1H), 8.20 (d, J=8.2 Hz, 1H), 7.92 (d, J=12.6 Hz, 1H), 7.78 (d, J=8.2 Hz, 1H), 7.44-7.35 (m, 2H), 7.24 (t, J=8.8 Hz, 1H), 4.33 (s, 2H), 4.09 (s, 1H), 4.01 (s, 1H), 3.70-3.52 (m, 4H), 3.42 (s, 2H), 3.20 (s, 2H), 2.11 (s, 3H).

### Synthesis of Example 59: 4-(3-(3-(2-Ethylphenyl)-5,6,7,8-tetrahydro-[1,2,4]triazolo[4,3-a]pyrazine-7-carbonyl)-4-fluorobenzyl)-6-(prop-1-ynyl)phthalazin-1(2H)-one

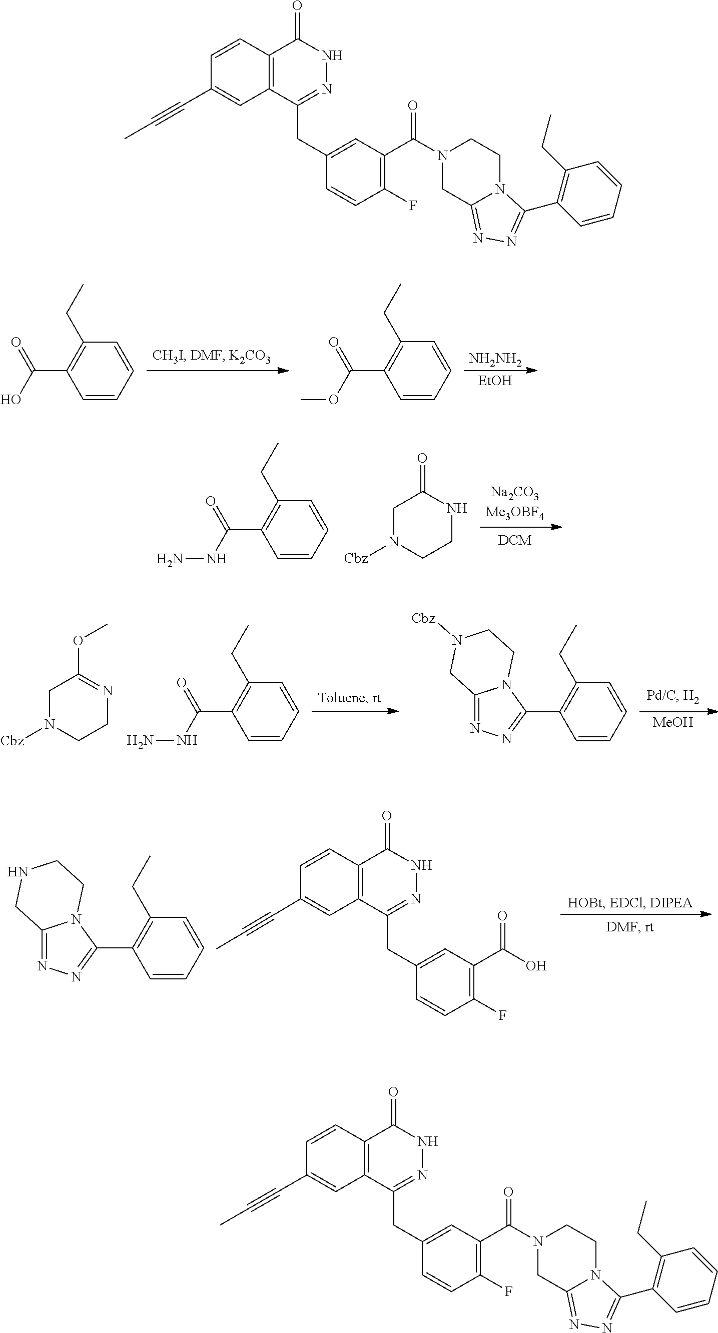

### Step 1

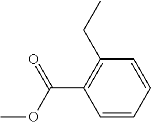

### Methyl 2-ethylbenzoate

Into a 40 ml vial were added 2-ethylbenzoic acid (5.00 g, 33.3 mmol), DMF (20 mL), iodomethane (5.67 g, 40.0 mmol, 2.49 mL) and potassium carbonate (13.8 g, 99.9 mmol, 6.03 mL) at room temperature. The mixture was stirred for 3.5 hr at 75° C. The mixture was extracted with Ethyl Acetate, washed with water. The organic phase was dried over Na₂SO₄, concentrated under reduced pressure to give methyl 2-ethylbenzoate (4.8 g, 88% yield) as a white solid. ¹H NMR (400 MHz, DMSO-d6) δ 7.76 (dd, J=7.8, 1.3 Hz, 1H), 7.50 (td, J=7.6, 1.4 Hz, 1H), 7.38-7.26 (m, 2H), 3.83 (s, 3H), 2.88 (q, J=7.5 Hz, 2H), 1.16 (m, 3H).

### Step 2

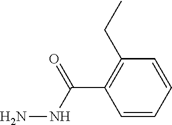

### 2-Ethylbenzohydrazide

A mixture of methyl 2-ethylbenzoate (4 g, 24.36 mmol) and Hydrazine monohydrate (9.76 g, 194.88 mmol, 9.50 mL) in EtOH (4 mL) was stirred at 100° C. under microwave for 3 hr. The reaction solution was concentrated under reduced pressure, diluted with DCM, washed with water. The organic phase was dried over Na₂SO₄ and concentrated to give 2-ethylbenzohydrazide (2.56 g, 64% yield) as a white solid. ¹H NMR (400 MHz, DMSO-d6) δ 9.40 (s, 1H), 7.30 (m, 4H), 4.43 (d, J=4.0 Hz, 2H), 2.69 (q. J=7.5 Hz, 2H), 1.14 (t, J=7.5 Hz, 3H).

### Step 3

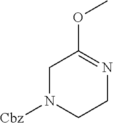 [SMILES: COC1=NCCN(C(=O)OCc2ccccc2)C1]

### Benzyl 3-methoxy-5,6-dihydropyrazine-1(2H)-carboxylate

To a solution of benzyl 3-oxopiperazine-1-carboxylate (5 g, 21.34 mmol) in DCM (200 mL) were added trimethyloxonium tetrafluoroborate (11.05 g, 74.71 mmol) and Na₂CO₃ (45.25 g, 426.89 mmol) at room temperature. The mixture was stirred at room temperature for 6 hr. The mixture was poured into water. The organic layer was separated, dried over Na₂SO₄ and concentrated under reduced pressure. The residue was purified by silica gel chromatography (eluent) to give benzyl 3-methoxy-5,6-dihydropyrazine-1(2H)-carboxylate (1 g, 19% yield) as a colorless oil. ¹H NMR (400 MHz, CDCl₃) δ 7.40-7.29 (m, 5H), 5.16 (s, 2H), 3.96 (s, 2H), 3.68 (s, 3H), 3.54 (s, 2H), 3.46 (m, 2H).

### Step 4

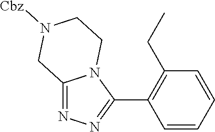 [SMILES: CCc1ccccc1-c1nnc2n1CCN(C(=O)OCc1ccccc1)C2]

### Benzyl 3-(2-ethylphenyl)-5,6-dihydro-[1,2,4]triazolo[4,3-a]pyrazine-7(8H)-carboxylate

Into a 40 mL sealed tube were added benzyl 6-methoxy-3,5-dihydro-2H-pyrazine-4-carboxylate (1.2 g, 4.83 mmol), toluene (70 mL) and 2-ethylbenzohydrazide (793.65 mg, 4.83 mmol) at room temperature. The solution was stirred overnight. The reaction system was concentrated under reduced pressure to remove the solvent. The residue was purified by silica gel chromatography (eluent) to give benzyl benzyl 3-(2-ethylphenyl)-5,6-dihydro-[1,2,4]triazolo[4,3-a]pyrazine-7(8H)carboxylate (1.5 g, 86% yield) as a yellow oil. LCMS ESI m/z: 363.2 [M+H]⁺.

### Step 5

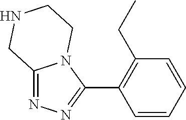 [SMILES: CCc1ccccc1-c1nnc2n1CCNC2]

### 3-(2-Ethylphenyl)-5,6,7,8-tetrahydro-[1,2,4]triazolo[4,3-a]pyrazine

A mixture of benzyl 3-(2-ethylphenyl)-5,6-dihydro-[1,2,4]triazolo[4,3-a]pyrazine-7(8H)-carboxylate (750 mg, 1.66 mmol) and Pd/C (500 mg, 4.12 mmol) in MeOH (5 mL) was stirred at room temperature for 2 h under hydrogen atmosphere. The mixture was filtered and concentrated to give 3-(2-ethylphenyl)-5,6,7,8-tetrahydro-[1,2,4]triazolo[4,3-a]pyrazine (320 mg, 85% yield) as a yellow oil. LCMS ESI m/z: 229.2 [M+H]⁺.

### Step 6

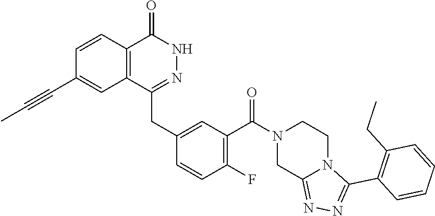 [SMILES: CC#Cc1ccc2c(=O)[nH]nc(Cc3ccc(F)c(C(=O)N4CCn5c(nnc5-c5ccccc5CC)C4)c3)c2c1]

### 4-(3-(3-(2-Ethylphenyl)-5,6,7,8-tetrahydro-[1,2,4]triazolo[4,3-a]pyrazine-7-carbonyl)-4-fluorobenzyl)-6-(prop-1-ynyl)phthalazin-1(2H)-one

A mixture of 2-fluoro-5-[(4-oxo-7-prop-1-ynyl-3H-phthalazin-1-yl)methyl]benzoic acid (50 mg, 148.67 μmol), 3-(2-ethylphenyl)-5,6,7,8-tetrahydro-[1,2,4]triazolo[4,3-a]pyrazine (45 mg, 197.12 μmol), DIPEA (77 mg, 596.90 μmol), EDCI (43 mg, 225.13 μmol) and HOBt (30.2 mg, 223.51 μmol) in DMF (3 mL) was stirred at room temperature for 3 hr under nitrogen atmosphere. The mixture was extracted with Ethyl Acetate, washed with water. The organic phase was dried over Na₂SO₄, concentrated under reduced pressure. The residue was purified by prep-HPLC to give 4-(3-(3-(2-ethylphenyl)-5,6,7,8-tetrahydro-[1,2,4]triazolo[4,3-a]pyrazine-7-carbonyl)-4-fluorobenzyl)-6-(prop-1-ynyl)phthalazin-1(2H)-one (5.0 mg, 6.1% yield) as a white solid. LCMS ESI m/z: 547.2 [M+H]⁺. ¹H NMR (400 MHz, DMSO-d₆) δ 12.59 (s, 1H), 8.17 (m, J=27.4, 8.2 Hz, 1H), 7.88 (d, J=50.9 Hz, 1H), 7.80-7.61 (m, 1H), 7.48 (dt, J=17.5, 7.4 Hz, 31H), 7.43-7.23 (m, 4H), 5.01 (s, 1H) 4.69 (s, 1H), 4.39-4.28 (m, 2H), 3.95 (d, J=91.4 Hz, 1H), 3.61 (s, 2H), 2.55 (q, J=7.1 Hz, 2H), 2.13-2.00 (m, 3H), 1.10-0.99 (m, 3H).

### Synthesis of Example 60: 6-(6-(2-Fluoro-5-((4-oxo-7-(prop-1-yn-1-yl)-3,4-dihydrophthalazin-1-yl)methyl)benzoyl)-2,6-diazaspiro[3.3]heptan-2-yl)nicotinonitrile

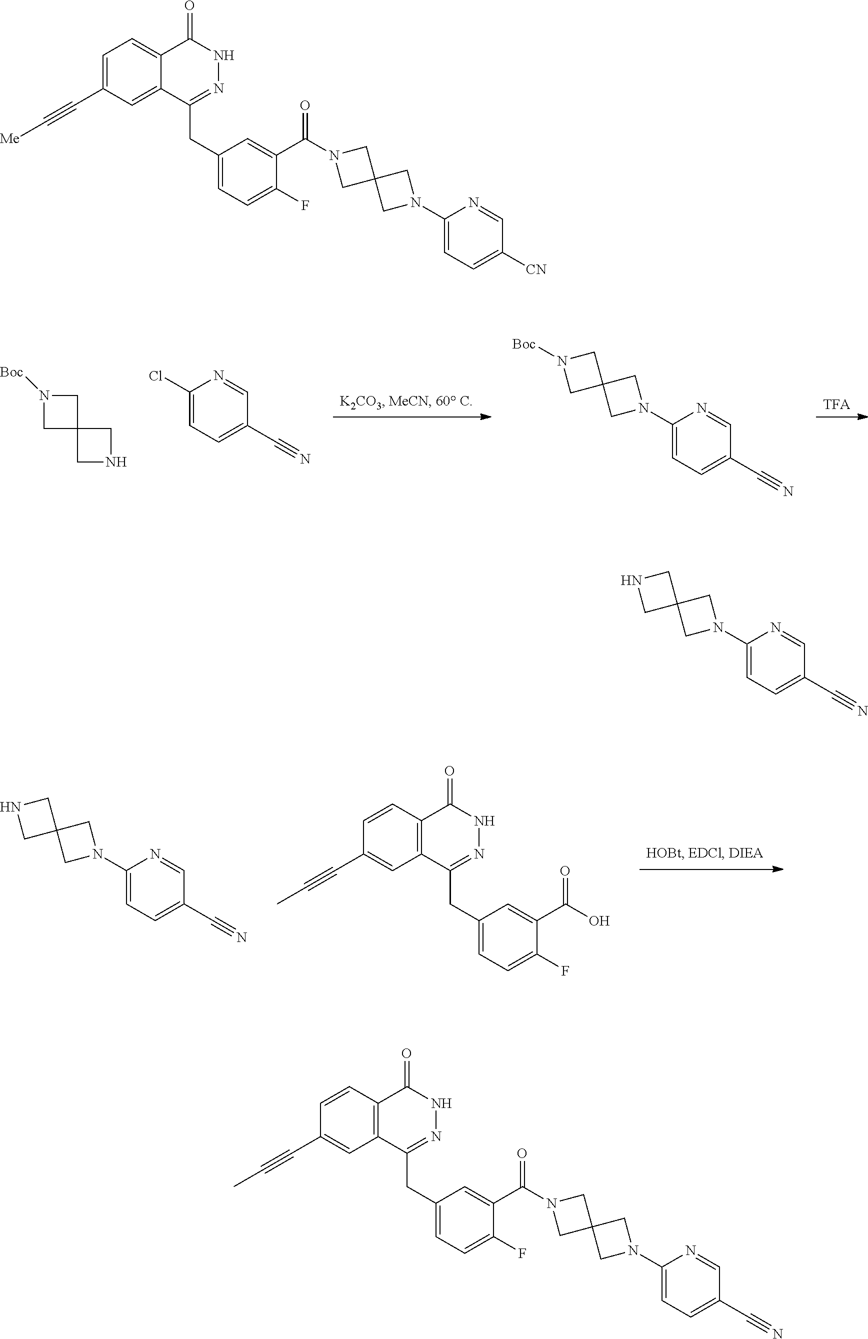

### Step 1

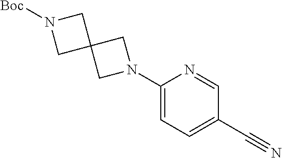 [SMILES: CC(C)(C)OC(=O)N1CC2(C1)CN(c1ccc(C#N)cn1)C2]

### tert-Butyl 6-(5-cyanopyridin-2-yl)-2,6-diazaspiro[3.3]heptane-2-carboxylate

A mixture of 6-chloropyridine-3-carbonitrile (476 mg, 3.43 mmol), tert-butyl 2,6-diazaspiro[3.3]heptane-2-carboxylate (683 mg, 3.43 mmol) and K₂CO₃ (1.19 g, 8.59 mmol) in MeCN (10 mL) was stirred at 60° C. for 16 hours. The reaction was diluted with H₂O (20 mL) and extracted with EtOAc (20 mL×3). The combined organic layer was washed with brine, dried over Na₂SO₄, filtered and concentrated in vacuo to afford tert-butyl 6-(5-cyanopyridin-2-yl)-2,6-diazaspiro[3.3]heptane-2-carboxylate as a white solid (1.03 g, 92% yield). This was used without further purification. LCMS ESI m/z: 301 [M+H]⁺.

### Step 2:

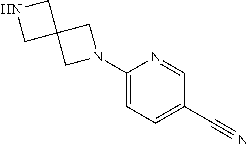 [SMILES: N#Cc1ccc(N2CC3(CNC3)C2)nc1]

### 6-(2,6-Diazaspiro[3.3]heptan-2-yl)nicotinonitrile

A solution of tert-butyl 6-(5-cyanopyridin-2-yl)-2,6-diazaspiro[3.3]heptane-2-carboxylate (400 mg, 1.33 mmol) in DCM (5 mL) and TFA (1 mL) was stirred at rt for 1 hour. The mixture was removed in vacuo to give 6-(2,6-diazaspiro[3.3]heptan-2-yl)nicotinonitrile as a yellow oil (266 mg, 99% yield). This was used without further purification. LCMS ESI m/z: 201 [M+H]⁺.

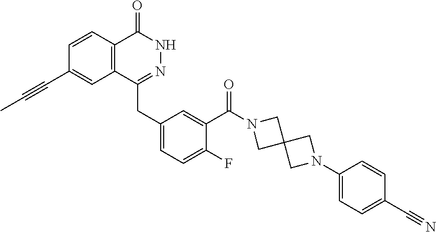 [SMILES: CC#Cc1ccc2c(=O)[nH]nc(Cc3ccc(F)c(C(=O)N4CC5(C4)CN(c4ccc(C#N)cc4)C5)c3)c2c1]

### 6-(6-(2-Fluoro-5-((4-oxo-7-(prop-1-yn-1-yl)-3,4-dihydrophthalazin-1-yl)methyl)benzoyl)-2,6-diazaspiro[3.3]heptan-2-yl)nicotinonitrile

To a solution of 6-(2,6-diazaspiro[3.3]heptan-2-yl)nicotinonitrile (89 mg, 0.45 mmol) and 2-fluoro-5-((4-oxo-7-(prop-1-yn-1-yl)-3,4-dihydrophthalazin-1-yl)methyl)benzoic acid (50 mg, 0.15 mmol) in DMF (1 mL) was added EDCI (43 mg, 0.22 mmol), HOB (30 mg, 0.22 mmol) and DIPEA (96 mg, 0.75 mmol), The reaction mixture was stirred at rt for 2 hours, then purified by prep-HPLC to give 6-(6-(2-fluoro-5-((4-oxo-7-(prop-1-yn-1-yl)-3,4-dihydrophthalazin-1-yl)methyl)benzoyl)-2,6-diazaspiro[3.3]heptan-2-yl)nicotinonitrile as a white solid (45 mg, 58% yield). LCMS ESI m/z: 519 [M+H]⁺. ¹H NMR (400 MHz, CDCl₃) δ 9.84 (s, 1H), 8.39 (d, J=1.6 Hz, 1H), 8.36 (d, J=8.2 Hz, 1H), 7.72 (d, J=8.2 Hz, 1H), 7.70 (s, 1H), 7.61 (dd, J=8.8, 2.2 Hz, 1H), 7.52-7.49 (m, 1H), 7.38-7.31 (m, 1H), 7.09-7.04 (m, 1H), 6.24 (d, J=8.7 Hz, 1H), 4.38 (s, 2H), 4.31-4.21 (m, 8H), 2.10 (s, 3H).

### Synthesis of Example 61: 6-(4-(2-Fluoro-5-((4-oxo-7-(prop-1-yn-1-yl)-3,4-dihydrophthalazin-1-yl)methyl)benzoyl)-3-methylpiperazin-1-yl)nicotinonitrile

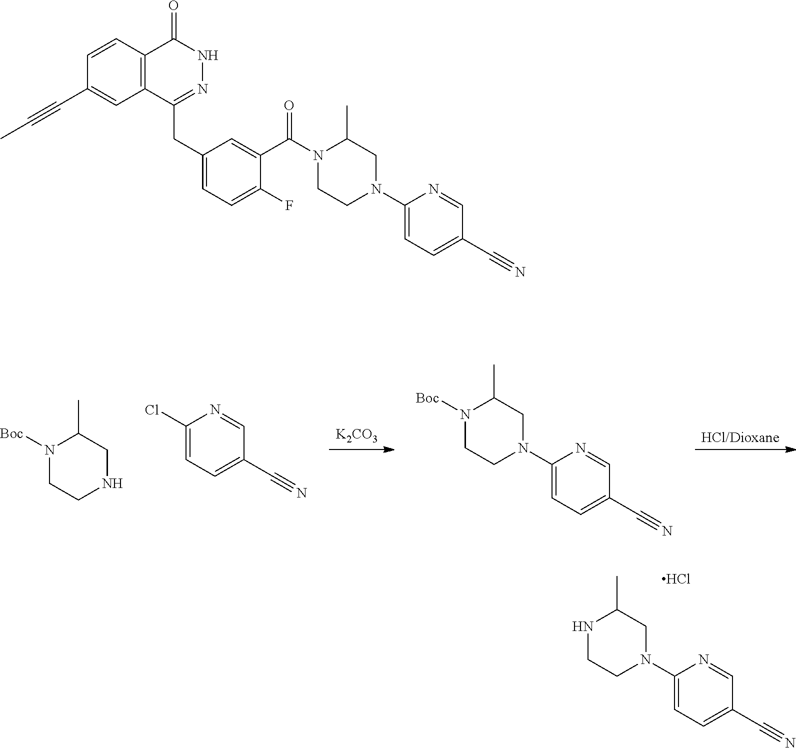

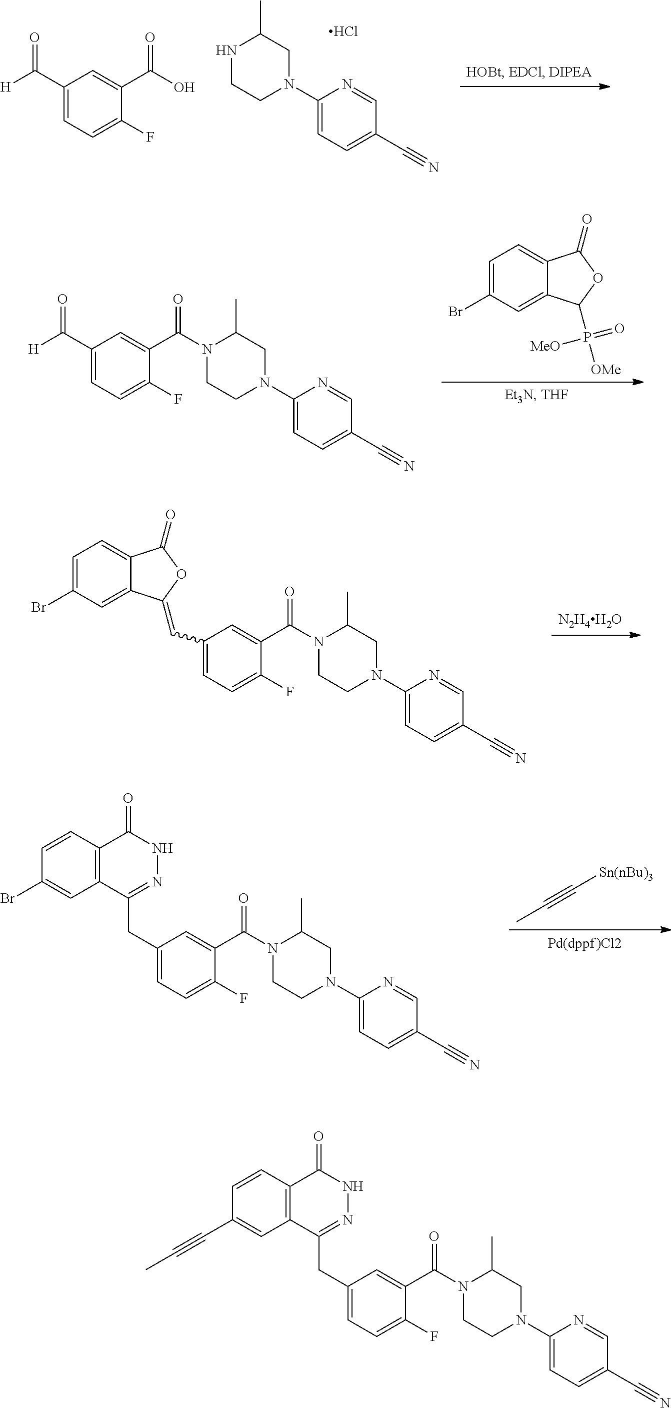

### Step 1

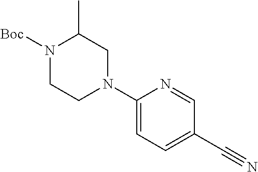 [SMILES: CC1CN(c2ccc(C#N)cn2)CCN1C(=O)OC(C)(C)C]

### tert-Butyl 4-(5-cyanopyridin-2-yl)-2-methylpiperazine-1-carboxylate

A mixture of 6-chloronicotinonitrile (500 mg, 3.61 mmol), tert-butyl 2-methylpiperazine-1-carboxylate (730 mg, 3.64 mmol) and K₂CO₃ (848 mg, 6.13 mmol) in MeCN (20 mL) was stirred at 60° C. for 12 hours. The reaction mixture was diluted with water (20 mL) and extracted with EtOAc (20 mL×3). The combined organic layer was washed with brine, dried over Na₂SO₄, filtered and concentrated in vacuo to afford tert-butyl 4-(5-cyanopyridin-2-yl)-2-methylpiperazine-1-carboxylate as a white solid (700 mg, 63% yield). This was used without further purification. LCMS ESI m/z: 247 [M-Boc+H]⁺.

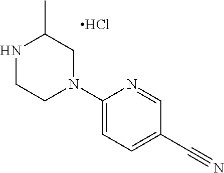

### 6-(3-Methylpiperazin-1-yl)nicotinonitrile hydrochloride

To a solution of tert-butyl 4-(5-cyanopyridin-2-yl)-2-methylpiperazine-1-carboxylate (700 mg, 2.32 mmol) in 1,4-dioxane (10 mL) was added HCl/1,4-dioxane (4M, 10 mL). The mixture was stirred at rt for 1 hour. Solvent was removed in vacuo to afford 6-(3-methylpiperazin-1-yl)nicotinonitrile hydrochloride as a white solid (450 mg, 96% yield). This was used without further purification. LCMS ESI m/z: 203 [M+H]⁺.

### Step 3

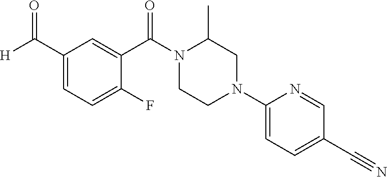 [SMILES: CC1CN(c2ccc(C#N)cn2)CCN1C(=O)c1cc(C=O)ccc1F]

### 6-(4-(2-fluoro-5-formylbenzoyl)-3-methylpiperazin-1-yl)nicotinonitrile

To a solution of 6-(3-methylpiperazin-1-yl)nicotinonitrile (450 mg, 2.22 mmol) and 2-fluoro-5-formylbenzoic acid (549 mg, 3.26 mmol) in DMF (5 mL) was added EDCI (663 mg, 4.08 mmol), HOBt (467 mg, 3.46 mmol) and DIPEA (893 mg, 8.16 mmol), The reaction mixture was stirred at rt for 12 hours, then diluted with water (20 mL) and extracted with EtOAc (20 mL×3). The combined organic layer was washed with brine, dried over Na₂SO₄, filtered and concentrated in vacuo to afford 6-(4-(2-fluoro-5-formylbenzoyl)-3-methylpiperazin-1-yl)nicotinonitrile as a white solid (636 mg, 81% yield). LCMS ESI m/z: 353 [M+H]⁺.

### Step 4

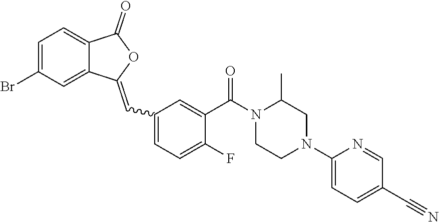 [SMILES: CC1CN(c2ccc(C#N)cn2)CCN1C(=O)c1cc(C=C2OC(=O)c3ccc(Br)cc32)ccc1F]

### 6-(4-(5-((6-Bromo-3-oxoisobenzofuran-1(3H)-ylidene)methyl)-2-fluorobenzoyl)-3-methylpiperazin-1-yl)nicotinonitrile

A solution of 6-(4-(2-fluoro-5-formylbenzoyl)-3-methylpiperazin-1-yl)nicotinonitrile (336 mg, 0.99 mmol), dimethyl (6-bromo-3-oxo-1,3-dihydroisobenzofuran-1-yl)phosphonate (270 mg, 0.84 mmol) and Et₃N (252 mg, 2.49 mmol) in THF (5 mL) was stirred at rt under Ar (g) for 12 hours. The reaction was diluted with water (20 mL) and extracted with EtOAc (20 mL×3). The combined organic layer was washed with brine, dried over Na₂SO₄, filtered and concentrated in vacuo to afford 6-(4-(5-((6-bromo-3-oxoisobenzofuran-1(3H)-ylidene)methyl)-2-fluorobenzoyl)-3-methylpiperazin-1-yl)nicotinonitrile as a white solid (420 mg, 94% yield). This was used without further purification. LCMS ESI m/z: 539 (M+H)⁺.

### Step 5

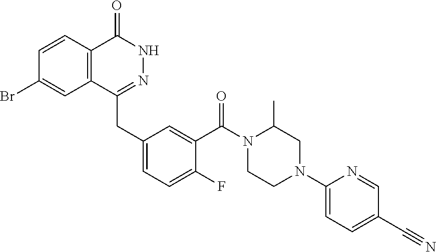 [SMILES: CC1CN(c2ccc(C#N)cn2)CCN1C(=O)c1cc(Cc2n[nH]c(=O)c3ccc(Br)cc23)ccc1F]

### 6-(4-(5-((7-Bromo 4-oxo-3,4-dihydrophthalazin-1-yl)methyl)-2-fluorobenzoyl)-3-methylpiperazin-1-yl)nicotinonitrile

A mixture of 6-(4-(5-((6-bromo-3-oxoisobenzofuran-(3H)-ylidene)methyl)-2-fluorobenzoyl)-3-methylpiperazin-1-yl)nicotinonitrile (340 mg, 0.62 mmol) and N₂H₄·H₂O (366 mg, 6.2 mmol) in THF (5 mL) was stirred at 70° C. for 3 hours. The solids were filtered and washed with water (10 mL) to afford 6-(4-(5-((7-bromo-4-oxo-3,4-dihydrophthalazin-1-yl)methyl)-2-fluorobenzoyl)-3-methylpiperazin-1-yl)nicotinonitrile as a white solid (302 mg, 87% yield). LCMS ESI m/z: 561 (M+H)⁺.

### Step 6

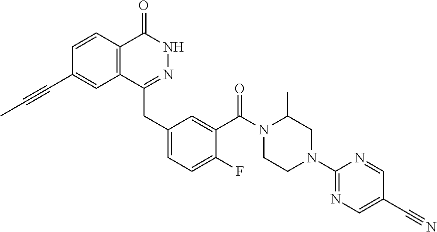 [SMILES: CC#Cc1ccc2c(=O)[nH]nc(Cc3ccc(F)c(C(=O)N4CCN(c5ncc(C#N)cn5)CC4C)c3)c2c1]

### 6-(4-(2-Fluoro-5-((4-oxo-7-(prop-1-yn-1-yl)-3,4-dihydrophthalazin-1-yl)methyl)benzoyl)-3-methylpiperazin-1-yl)nicotinonitrile

A mixture of tributyl(prop-1-ynyl)stannane (176 mg, 0.53 mmol), 6-(4-(5-((7-bromo-4-oxo-3,4-dihydrophthalazin-1-yl)methyl)-2-fluorobenzoyl)-3-methylpiperazin-1-yl)nicotinonitrile (100 mg, 0.18 mmol) and Pd(dppf)Cl₂ (13 mg, 0.02 mmol) in 1,4-dioxane (5 mL) was stirred at 100° C. under Ar (g) for 16 hours. The reaction mixture was concentrated in vacuo and the residue was purified by prep-HPLC to afford 6-(4-(2-fluoro-5-((4-oxo-7-(prop-1-yn-1-yl)-3,4-dihydrophthalazin-1-yl)methyl)benzoyl)-3-methylpiperazin-1-yl)nicotinonitrile as a white solid (32 mg, 35% yield). LCMS ESI m/z: 521 [M+H]⁺; ¹H NMR (400 MHz, CDCl₃) δ 10.48 (s, 1H), 8.46-8.31 (m, 2H), 7.71 (d, J=7.3 Hz, 2H), 7.63 (dd, J=9.0, 2.2 Hz, 1f), 7.32 (d, J=8.5 Hz, 2H), 7.07 (t, J=8.8 Hz, 1H), 6.59 (t, J=7.9 Hz, 1H), 5.10-4.59 (m, 1H), 4.45-4.14 (m, 4H), 3.90-2.99 (m, 4H), 2.09 (s, 3H), 1.30 (d, J=6.6 Hz, 3H).

### Synthesis of Example 62: 6-(8-(2-Fluoro-5-((4-oxo-7-(prop-1-yn-1-yl)-3,4-dihydrophthalazin-1-yl)methyl)benzoyl)-3,8-diazabicyclo[3.2.1]octan-3-yl)nicotinonitrile

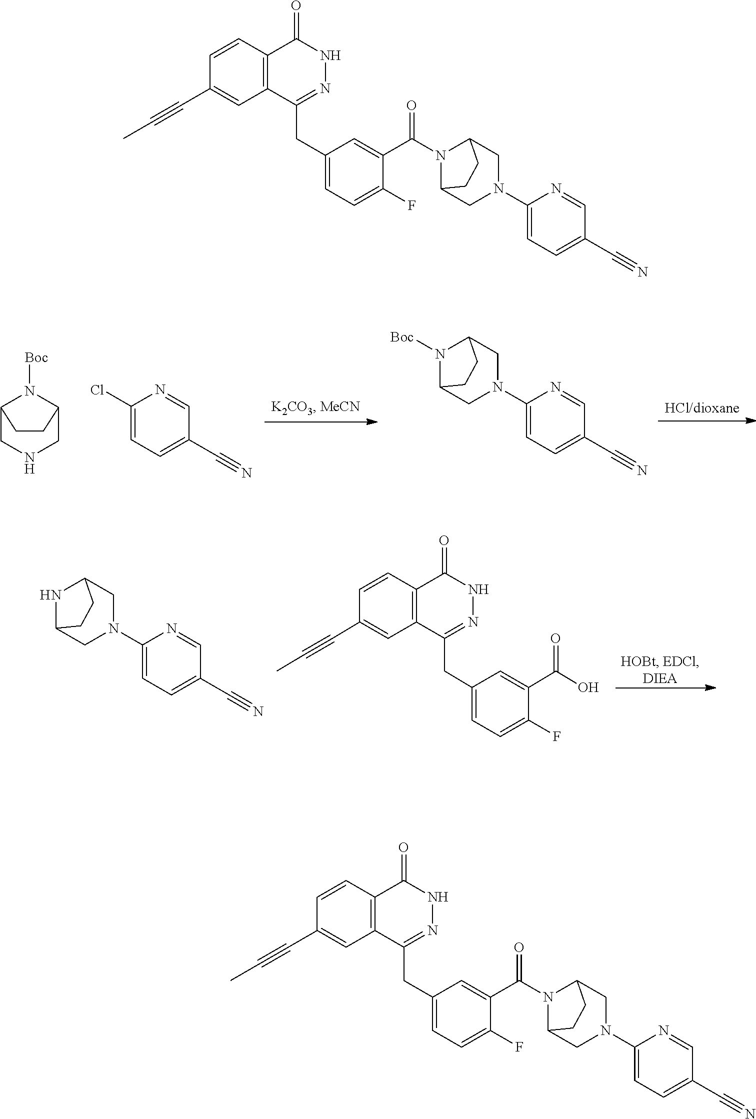

### Step 1

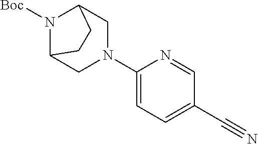 [SMILES: CC(C)(C)OC(=O)N1C2CCC1CN(c1ccc(C#N)cn1)C2]

### tert-Butyl 3-(5-cyanopyridin-2-yl)-3,8-diazabicyclo[3.2.1]octane-8-carboxylate

A mixture of 6-chloronicotinonitrile (500 mg, 3.61 mmol), tert-butyl-3,8-diazabicyclo[3.2.1]octane-8-carboxylate (766 mg, 3.61 mmol) and K₂CO₃ (848 mg, 6.13 mmol) in MeCN (10 mL) was stirred at 60° C. for 12 hours. The mixture was diluted with water (20 mL) and extracted with EtOAc (20 mL×3). The combined organic layer was washed with brine, dried over Na₂SO₄, filtered and concentrated in vacuo to afford tert-butyl 3-(5-cyanopyridin-2-yl)-3,8-diazabicyclo[3.2.1]octane-8-carboxylate as a white solid (1.02 g, 90% yield). This was used without further purification. LCMS ESI m/z: 247 [M+H]⁺.

### Step 2

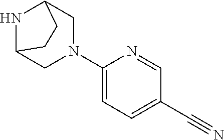 [SMILES: N#Cc1ccc(N2CC3CCC(C2)N3)nc1]

### 6-(3,8-Diazabicyclo[3.2.1]octan-3-yl)nicotinonitrile

To a solution of tert-butyl 3-(5-cyanopyridin-2-yl)-3,8-diazabicyclo[3.2.1]octane-8-carboxylate (200 mg, 0.64 mmol) in 1,4-dioxane (1 mL) was added HCl/dioxane (4M, 1 mL). The mixture was stirred at rt for 1 hour. Solvent was removed in vacuo to afford 6-(3,8-diazabicyclo[3.2.1]octan-3-yl)nicotinonitrile as a white solid (130 mg, 96% yield). This was used without further purification. LCMS ESI m/z: 215 [M+H]⁺.

### Step 3

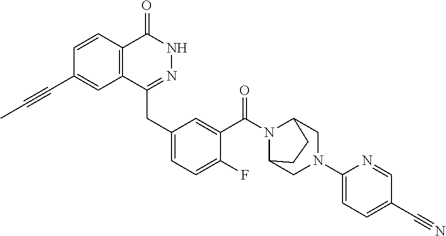 [SMILES: CC#Cc1ccc2c(=O)[nH]nc(Cc3ccc(F)c(C(=O)N4C5CCC4CN(c4ccc(C#N)cn4)C5)c3)c2c1]

### 6-(8-(2-Fluoro-5-((4-oxo-7-(prop-1-yn-1-yl)-3,4-dihydrophthalazin-1-yl)methyl)benzoyl)-3,8-diazabicyclo[3.2.1]octan-3-yl)nicotinonitrile

To a solution of 6-(3,8-diazabicyclo[3.2.1]octan-3-yl)nicotinonitrile (100 mg, 0.40 mmol) and 2-fluoro-54(4-oxo-7-(prop-1-yn-1-yl)-3,4-dihydrophthalazin-1-yl)methyl)benzoic acid (201 mg, 0.60 mmol) in DMF (2 mL) was added EDCI (114 mg, 0.60 mmol), HOBt (81 mg, 0.60 mmol) and DIPEA (155 mg, 1.20 mmol), The reaction was stirred at rt for 2 h. The reaction mixture was purified by prep-HPLC to afford 6-(8-(2-fluoro-5-((4-oxo-7-(prop-1-yn-1-yl)-3,4-dihydrophthalazin-1-yl)methyl)benzoyl)-3,8-diazabicyclo[3.2.1]octan-3-yl)nicotinonitrile as a white solid (55 mg, 26% yield). LCMS ESI m/z: 533 [M+H]⁺): ¹H NMR (400 MHz, DMSO-d₆) δ 12.63 (s, 1H), 8.51 (d, J=2.3 Hz, 1H), 8.19 (d, J=8.2 Hz, 1H), 7.94 (s, 1H), 7.90 (dd, J=9.1, 2.3 Hz, 1H), 7.79-7.76 (m, 1H), 7.51-7.46 (m, 1H), 7.45-7.39 (m, 1H), 7.29-7.23 (m, 1H), 6.85 (d, J=9.1 Hz, 1H), 4.83-4.77 (m, 1H), 4.34 (s, 2H), 4.26 (d, J=11.5 Hz, 1H), 4.10 (d, J=11.2 Hz, 1H), 3.87-3.81 (m, 1H), 3.14 (d, J=11.3 Hz, 1H), 2.92 (d, J=12.3 Hz, 1H), 2.07 (s, 3H), 1.93-1.80 (m, 2H), 1.71-1.58 (m, 2H).

### Synthesis of Example 63: 4-[[3-(3-cyclopropyl-6,8-dihydro-5H-[1,2,4]triazolo[4,3-a]pyrazine-7-carbonyl)-4-fluoro-phenyl]methyl]-6-prop-1-ynyl-2H-phthalazin-1-one

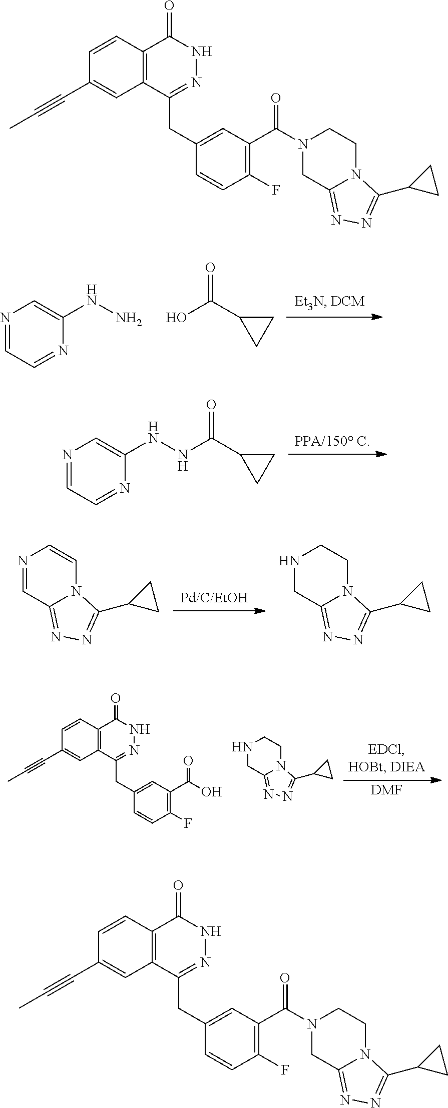

### Step 1

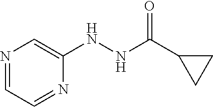 [SMILES: O=C(NNc1cnccn1)C1CC1]

### N′-pyrazin-2-ylcyclopropanecarbohydrazide

A mixture of pyrazin-2-ylhydrazine (1 g, 9.08 mmol) and Et₃N (1.84 g, 18.16 mmol, 2.53 mL) in DCM (12 mL) was stirred at rt. Cyclopropanecarbonyl chloride (1.04 g, 9.99 mmol, 908.03 μL) was added dropwise to the reaction mixture at room temperature under nitrogen atmosphere. The solution was stirred at room temperature for 3 hours. The solution was concentrated under reduced pressure. The residue was purified by silica gel chromatography (DCM:MeOH=12:1) to give N-pyrazin-2-ylcyclopropanecarbohydrazide (430 mg, 27% yield). LCMS ESI m/z: 179.1 [M+H]⁺.

### Step 2

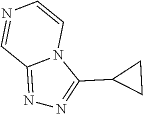 [SMILES: c1cn2c(C3CC3)nnc2cn1]

### 3-Cyclopropyl-[1,2,4]triazolo[4,3-a]pyrazine

A solution of N-pyrazin-2-ylcyclopropanecarbohydrazide (200 mg, 841.79 μmol) in PPA (3 m L) was stirred at 150° C. for 2 hours and then for 36 hours at 100° C. under nitrogen atmosphere. The mixture was worked up with EtOAc and water. The mixture was added ammonium hydroxide until reached pH 1. The organic phase was dried over Na₂SO₄, concentrated under reduced pressure. The residue was purified by Prep-TLC (DCM:MeOH=10:1) to give 3-cyclopropyl-[1,2,4]triazolo[4,3-a]pyrazine (80 mg, 59% yield) as a yellow solid. ¹H NMR (400 MHz, DMSO-d₆) δ 9.32 (d, J=1.6 Hz, 1H), 8.61 (dd, J=4.8, 1.6 Hz, 1H), 7.93 (d, J=4.8 Hz, 1H), 2.48-2.43 (m, 1H), 1.18 (ddd, J=11.6, 5.3, 3.2 Hz, 2H), 1.10 (qd, J=5.0, 2.2 Hz, 2H).

### Step 3

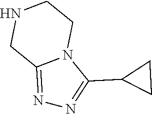 [SMILES: C1Cn2c(nnc2C2CC2)CN1]

### 3-Cyclopropyl-5,6,7,8-tetrahydro-[1,2,4]triazolo[4,3-a]pyrazine

A mixture of 3-cyclopropyl-[1,2,4]triazolo[4,3-a]pyrazine (80 mg, 499.45 μmol) and Pd/C (60.66 mg, wet) in EtOH (5 mL) was stirred at room temperature under hydrogen atmosphere for 16 hours. The mixture was filtered. The filtrate was concentrated under reduced pressure to give 3-cyclopropyl-5,6,7,8-tetrahydro-[1,2,4]triazolo[4,3-a]pyrazine (40 mg, 49% yield) as a colorless oil. LCMS ESI m/z: 165.2 [M+H]⁺.

### Step 4

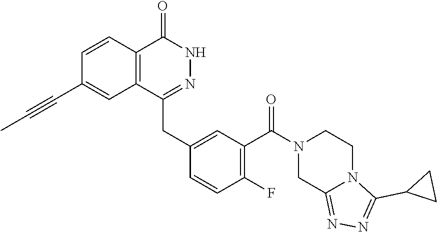 [SMILES: CC#Cc1ccc2c(=O)[nH]nc(Cc3ccc(F)c(C(=O)N4CCn5c(nnc5C5CC5)C4)c3)c2c1]

### 4-[[3-(3-Cyclopropyl-6,8-dihydro-5H-[1,2,4]triazol)[4,3-a]pyrazine-7-carbonyl)-4-fluoro-phenyl]methyl]-6-prop-1-ynyl-2H-phthalazin-1-one

A solution of 2-methyl-5-[(4-oxo-7-prop-1-ynyl-3H-phthalazin-1-yl)methyl]benzoic acid (99.40 mg, 299.07 μmol), 3-cyclopropyl-5,6,7,8-tetrahydro-[1,2,4]triazolo[4,3-a]pyrazine (152-4) (60 mg, 365.39 μmol), DIPEA (141.41 mg, 1.10 mmol), EDCI (104.68 mg, 548.09 μmol) and HOBt (74.06 mg, 548.09 μmol) in DMF (8 mL) was stirred at room temperature under nitrogen atmosphere for 3 hours. The solution was dissolved in EtOAc (20 mL), washed with water (10 mL×3). The organic phase was dried over Na₂SO₄, concentrated under reduced pressure. The residue was purified by Prep-TLC (DCM:MeOH=10:1) to give 4-[[3-(3-cyclopropyl-6,8-dihydro-5H-[1,2,4]triazolo[4,3-a]pyrazine-7-carbonyl)-4-fluoro-phenyl]methyl]-6-prop-1-ynyl-2H-phthalazin-1-one (8.5 mg, 4.8% yield) as a white solid. LCMS ESI 483.2 [M+H]⁺. ¹H NMR (400 MHz, CDCl₃) δ 10.52 (s, 1H), 8.35 (d, J=8.2 Hz, 1H), 7.71 (d, J=7.4 Hz, 2H), 7.47-7.31 (m, 2H), 7.11 (t, J=8.9 Hz, 1H), 4.92 (d, J=133.5 Hz, 2H), 4.32-3.70 (m, 6H), 2.10 (s, 3H), 1.70-1.65 (m, 1H), 1.13 (dd, J=7.2, 3.3 Hz, 2H), 1.06 (d, J=7.7 Hz, 2H).

### Synthesis of Example 64: 6-(3-(2-Fluoro-5-((4-oxo-7-(prop-1-ynyl)-3,4-dihydrophthalazin-1-yl)methyl)benzoyl)-3,8-diazabicyclo[3.2.1]octan-8-yl)nicotinonitrile

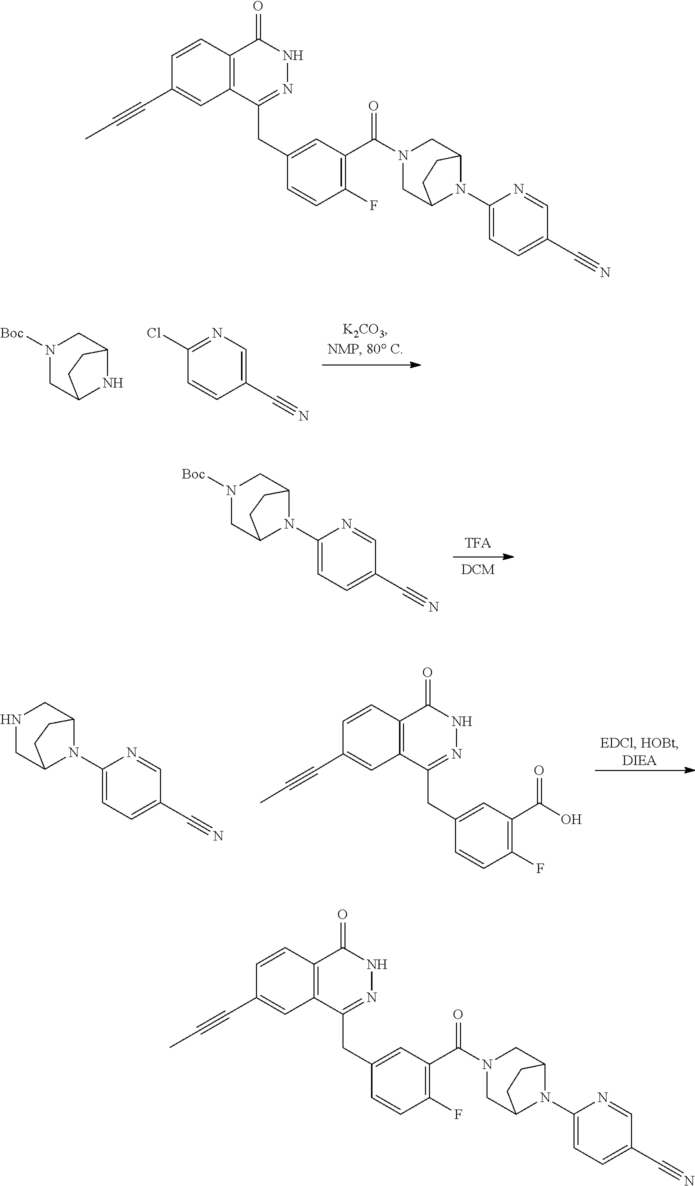

### Step 1, 2 and 3

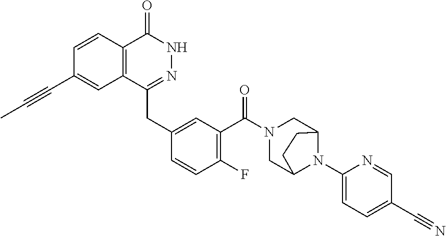 [SMILES: CC#Cc1ccc2c(=O)[nH]nc(Cc3ccc(F)c(C(=O)N4CC5CCC(C4)N5c4ccc(C#N)cn4)c3)c2c1]

### 6-(3-(2-Fluoro-5-((4-oxo-7-(prop-1-ynyl)-3,4-dihydrophthalazin-1-yl)methyl)benzoyl)-3,8-diazabicyclo[3.2.1]octan-8-yl)nicotinonitrile

Following the three-step procedure above in Example 55, but starting with tert-butyl 3,8-diazabicyclo[3.2.1]octane-3-carboxylate gave the title compound as a white solid. LCMS ESI m/z: 533.2 [M+H]⁺. ¹H NMR (400 MHz, DMSO-d6) δ12.61 (s, 1H), 8.52 (d, J=2.1 Hz, 1H), 8.19 (d, J=8.2 Hz, 1H), 7.90 (dd, J=9.0, 2.3 Hz, 2H), 7.78 (dd, J=8.2, 1.2 Hz, 1H), 7.50-7.29 (m, 2H), 7.24 (t, J=8.9 Hz, 1H), 6.90 (d, J=9.0 Hz, 1H), 4.80 (s, 1H), 4.60 (s, 1H), 4.33 (s, 3H), 3.27 (s, 1H), 3.11 (m, 1H), 2.99 (d, J=12.6 Hz, 1H), 2.09 (m, 3H), 2.01-1.84 (m, 2H), 1.75 (m, 2H).

### Synthesis of Example 65: Preparation of 4-(4-fluoro-3-(3-(trifluoromethyl)-5,6,7,8-tetrahydro-[1,2,4]triazolo[4,3-a]pyrazine-7-carbonyl)benzyl)-6-(prop-1-yn-1-yl)phthalazin-1(2H)-one

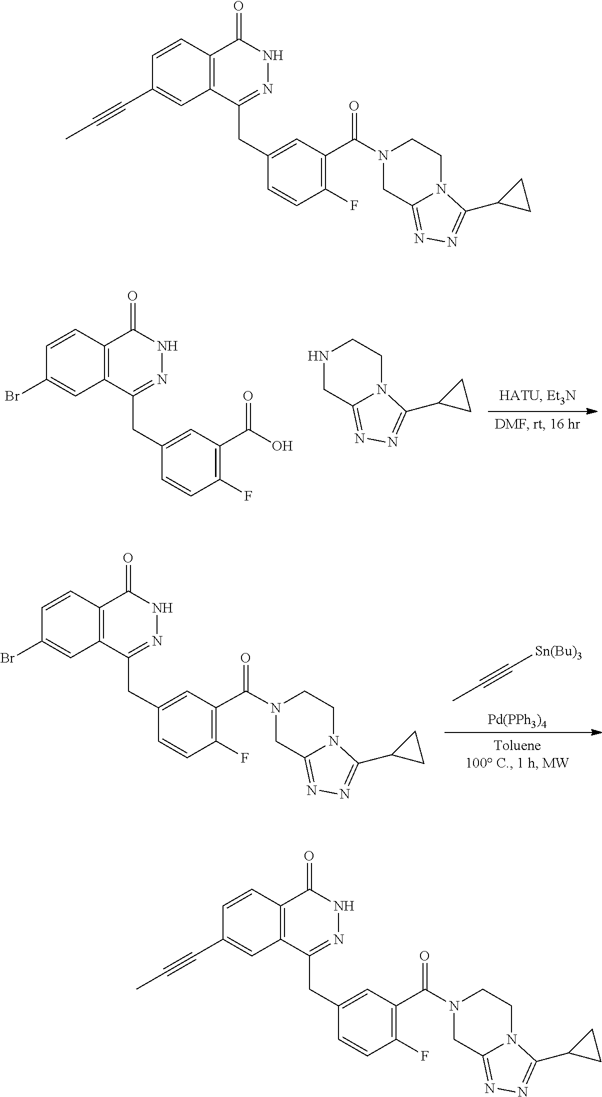

### Step 1

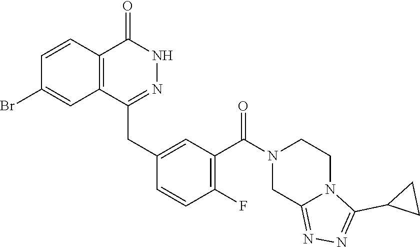 [SMILES: O=C(c1cc(Cc2n[nH]c(=O)c3ccc(Br)cc23)ccc1F)N1CCn2c(nnc2C2CC2)C1]

### 6-Bromo-4-(4-fluoro-3-(3-(trifluoromethyl)-5,6,7,8-tetrahydro-[1,2,4]triazolo[4,3-a]pyrazine-7-carbonyl)benzyl)phthalazin-1(2H)-one

To a solution of 5-[(7-bromo-4-oxo-3H-phthalazin-1-yl)methyl]-2-fluoro-benzoic acid (450 mg, 1.19 mmol) in DMF (10 mL) was added HATU (544.39 mg, 1.43 mmol) and Et₃N (482.92 mg, 4.77 mmol, 665.19 μL). The mixture was stirred at rt for 0.5 hour, 3-(trifluoromethyl)-5,6,7,8-tetrahydro-[1,2,4]triazolo[4,3-a]pyrazine (272.75 mg, 1.19 mmol) was added, then stirred at rt for another 16 h. The reaction mixture was poured into water (50 mL), extracted with EtOAc (50 mL×3). The extracts were washed with brine, dried and concentrated to give 6-bromo-4-[[4-fluoro-3-[3-(trifluoromethyl)-6,8-dihydro-5H-[1,2,4]triazolo[4,3-a]pyrazine-7-carbonyl]phenyl]methyl]-2H-phthalazin-1-one (700 mg, crude) as a white solid. LCMS ESI 548.7 [M−H]⁻.

### Step 2

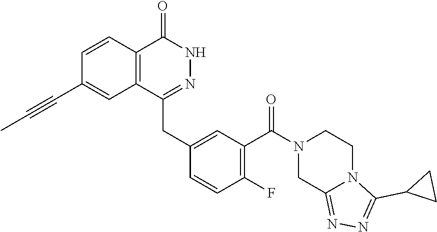 [SMILES: CC#Cc1ccc2c(=O)[nH]nc(Cc3ccc(F)c(C(=O)N4CCn5c(nnc5C5CC5)C4)c3)c2c1]

### 4-(4-Fluoro-3-(3-(trifluoromethyl)-5,6,7,8-tetrahydro-[1,2,4]triazolo[4,3-a]pyrazine-7-carbonyl)benzyl)-6-(prop-1-yn-1-yl)phthalazin-1(2H)-one

To a solution of 6-bromo-4-[[4-fluoro-3-[3-(trifluoromethyl)-6,8-dihydro-5H-[1,2,4]triazolo[4,3-a]pyrazine-7-carbonyl]phenyl]methyl]-2H-phthalazin-1-one (700 mg, 253.95 μmol) in PhCH₃ (4 mL) was added tributyl(prop-1-ynyl)stannane (835.77 mg, 2.54 mmol) and Pd(PPh₃)₄ (29.35 mg, 25.39 μmol) The mixture was stirred at 100° C. under microwave for 1 hour. CsF (1 g) and water (20 m L) were added to the reaction mixture. The mixture was stirred at rt for another 0.5 hour. The mixture was extracted with EtOAc (30 mL×3), dried and concentrated to the residue. The residue was purified by Prep-HPLC to give 4-[[4-fluoro-3-[3-(trifluoromethyl)-6,8-dihydro-5H-[1,2,4]triazolo[4,3-a]pyrazine-7-carbonyl]phenyl]methyl]-6-prop-1-ynyl-2H-phthalazin-1-one (21.28 mg, 16% yield) as a light yellow solid. LCMS ESI 511.2 [M+H]⁺. ¹H NMR (400 MHz, DMSO-d₆) δ 12.61 (s, 1H), 8.20 (d, J=8.2 Hz, 1H), 7.90 (d, J=21.3 Hz, 1H), 7.77 (dd, J=8.2, 1.3 Hz, 1H), 7.53-7.46 (m, 1H), 7.41 (d, J=5.5 Hz, 1H), 7.31 (t, J=8.8 Hz, 1H), 5.03 (m, 1H), 4.71 (m, 1H), 4.35 (s, 2H), 4.23 (m, 1H), 4.15 (m, 1H), 4.02 (m, 1H), 3.70 (m, 1H), 2.10 (s, 3H).

### Synthesis of Example 66: 6-((1R,4R)-5-(2-Fluoro-5-((4-oxo-7-(prop-1-ynyl)-3,4-dihydrophthalazin-1-yl)methyl)benzoyl)-2,5-diazabicyclo[2.2.1]heptan-2-yl)nicotinonitrile

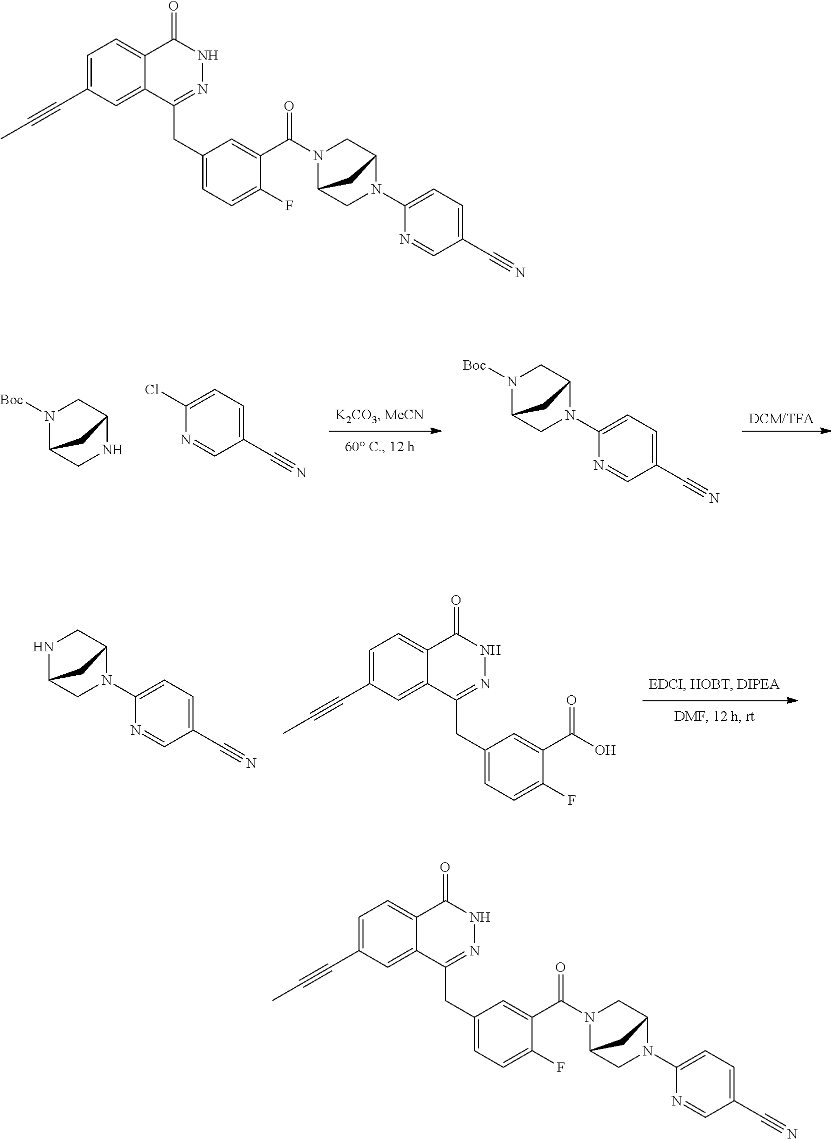

### Step 1 and 2

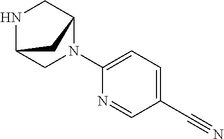 [SMILES: N#Cc1ccc(N2C[C@H]3C[C@@H]2CN3)nc1]

### 6-((1R,4R)-2,5-Diazabicyclo[2.2.1]heptan-2-yl)nicotinonitrile

Following the general procedure above in Example 55, but starting with (1R,4R)-tert-butyl 2,5-diazabicyclo[2.2.1]heptane-2-carboxylate and 6-chloronicotinonitrile gave the title compound as a colorless oil. LCMS ESI m/z: 201.1 [M+H]⁺.

### Step 3

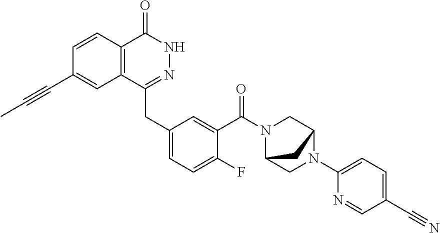 [SMILES: CC#Cc1ccc2c(=O)[nH]nc(Cc3ccc(F)c(C(=O)N4C[C@H]5C[C@@H]4CN5c4ccc(C#N)cn4)c3)c2c1]

### 6-((1R,4R)-5-(2-Fluoro-5-((4-oxo-7-(prop-1-ynyl)-3,4-dihydrophthalazin-1-yl)methyl)benzoyl)-2,5-diazabicyclo[2.2.1]heptan-2-yl)nicotinonitrile

Following the procedure in Example 55, but starting with 6-((1R,4R)-2,5-diazabicyclo[2.2.1]heptan-2-yl)nicotinonitrile gave the title compound as a white solid. LCMS ESI m/z: 518.7 [M+H]⁺. ¹H NMR (400 MHz, DMSO-D6) δ 12.61 (d, J=18.5 Hz, 1H), 8.51-8.41 (m, 1H), 8.20 (dd, J=12.8, 8.2 Hz, 1H), 7.96-7.73 (m, 3H), 7.53-7.11 (m, 3H), 6.59 (d, J=38.1 Hz, 1H), 4.98 (s, 1H), 4.33 (m, 2H), 4.19 (s, 1H), 3.67-3.56 (m, 1H), 3.42 (m, 3H), 2.10 (t, J=8.3 Hz, 3H), 1.97 (m, 2H).

### Synthesis of Example 67: 6-(4-Fluoro-1-(2-fluoro-5-((4-oxo-7-(prop-1-yn-1-yl)-3,4-dihydrophthalazin-1-yl)methyl)benzoyl)piperidin-4-yl)nicotinonitrile

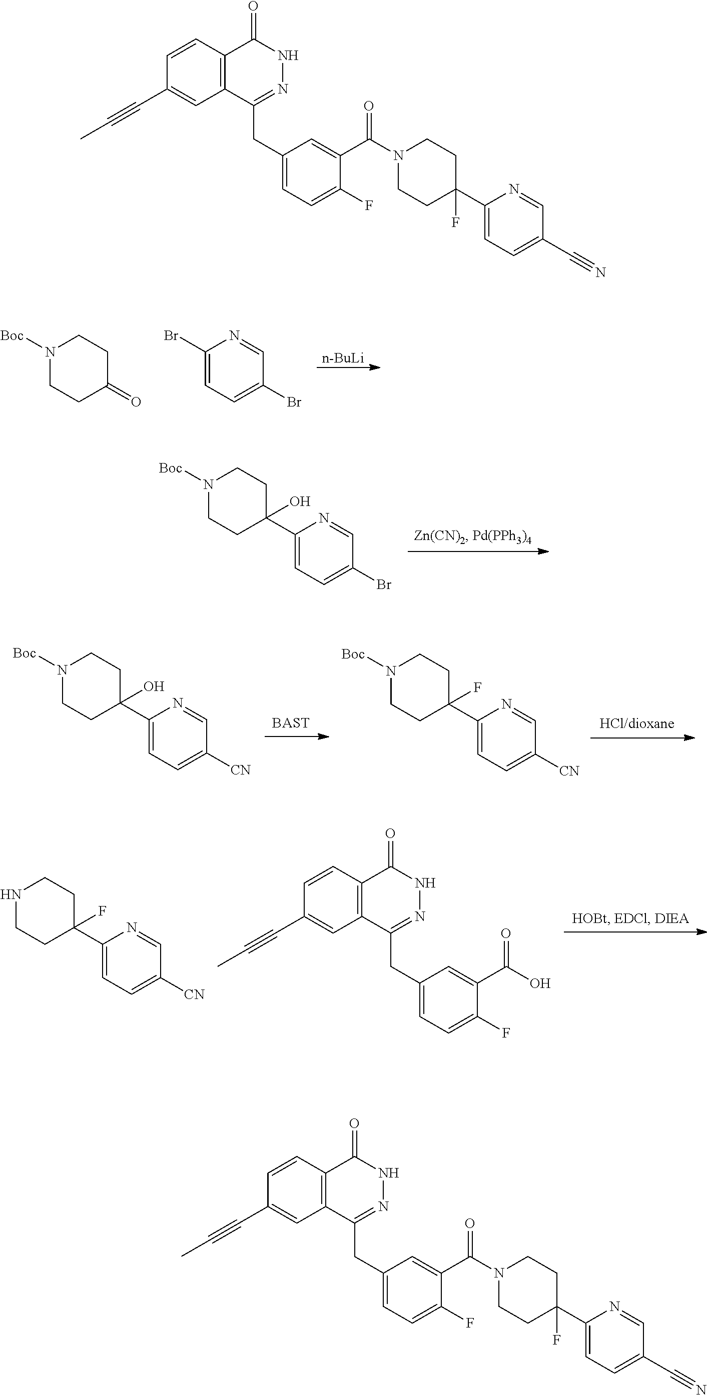

### Step 1

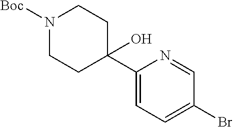 [SMILES: CC(C)(C)OC(=O)N1CCC(O)(c2ccc(Br)cn2)CC1]

### tert-Butyl 4-(5-cyanopyridin-2-yl)-4-hydroxypiperidine-1-carboxylate

A solution of 2,5-dibromopyridine (705 mg, 3 mmol) in THF (20 mL) was cooled to −78° C., then n-BuLi (2.5M in heptane, 1.32 mL, 3.3 mmol) was added dropwise under Ar (g). The reaction was stirred at −78° C. for 15 min, then tert-butyl 4-oxopiperidine-1-carboxylate (716 mg, 3.6 mmol) was added. The reaction was kept at −78° C. for 20 min, then quenched with sat. NH₄Cl solution (10 mL) and extracted with EtOAc (30×3 mL). The combined organic layer was washed with brine, dried over Na₂SO₄, filtered and concentrated in vacuo. The residue was purified by silica gel chromatography (EtOAc/pet ether=20 to 40%) to give tert-butyl 4-(5-bromopyridin-2-yl)-4-hydroxypiperidine-1-carboxylate as a white solid (520 mg, 49% yield). LCMS ESI 357 (M+H)⁺.

### Step 2

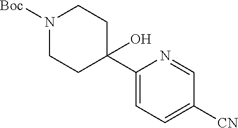 [SMILES: CC(C)(C)OC(=O)N1CCC(O)(c2ccc(C#N)cn2)CC1]

### tert-Butyl 4-(5-cyanopyridin-2-yl)-4-hydroxypiperidine-1-carboxylate

A mixture of tert-butyl 4-oxopiperidine-1-carboxylate (520 mg, 1.46 mol). Zn(CN): (347 mg, 2.92 mmol) and Pd(PPh₃)₄ in dry DMF (10 mL) was stirred at 100′° C. under Ar (g) for 2 hours. The mixture was cooled to rt, diluted with H₂O (10 mL) and extracted with EtOAc (20 mL×3). The combined organic layer was washed with brine, dried over anhydrous Na₂SO₄, filtered and concentrated in vacuo to give tert-butyl 4-(5-cyanopyridin-2-yl)-4-hydroxypiperidine-1-carboxylate as light yellow oil (380 mg, 86% yield). LCMS ESI 304 (M+H)⁺.

### Step 3: tert-Butyl 4-(5-cyanopyridin-2-yl)-4-fluoropiperidine-1-carboxylate

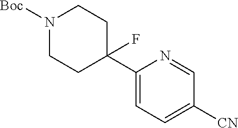 [SMILES: CC(C)(C)OC(=O)N1CCC(F)(c2ccc(C#N)cn2)CC1]

To a solution of tert-butyl 4-(5-cyanopyridin-2-yl)-4-hydroxypiperidine-1-carboxylate (380 mg, 1.25 mmol) in DCM (10 mL) was added BAST (966 mg, 4.37 mmol) in portions at 0° C. The reaction was stirred at rt for 12 hours. The mixture was diluted with H₂O (10 mL) and extracted with DCM (3×20 mL). The combined organic layer was dried over Na₂SO₄, filtered and concentrated in vacuo. The residue was purified by silica gel chromatography (EtOAc/pet ether=0 to 10%) to give tert-butyl 4-(5-cyanopyridin-2-yl)-4-fluoropiperidine-1-carboxylate as a yellow oil (170 mg, 45% yield). LCMS ESI m/z: 306 (M+H)⁺. ¹H NMR (40) MHz, CDCl₃) δ 8.20 (s, 1H), 8.02 (dd, J=8.0, 2.0 Hz, 1H), 7.73 (t, J=7.6 Hz, 1H), 4.15-4.05 (m, 4H), 3.18-3.03 (m, 2H), 2.38-2.15 (m, 2H), 1.49 (s, 9H).

### Step 4

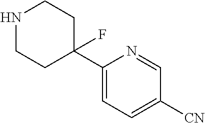 [SMILES: N#Cc1ccc(C2(F)CCNCC2)nc1]

### 6-(4-Fluoropiperidin-4-yl)nicotinonitrile

To a solution of tert-butyl 4-(5-cyanopyridin-2-yl)-4-fluoropiperidine-1-carboxylate (115 mg, 0.37 mmol) in 1,4-dioxane (1 mL) was added HCl/dioxane (4 M, 1 mL). The mixture was stirred at rt for 1 hour, then concentrated in vacuo to afford 6-(4-fluoropiperidin-4-yl)nicotinonitrile as a white solid (75 mg, 98% yield). This was used without further purification. LCMS ESI m/z: 206 [M+H]⁺.

### Step 5

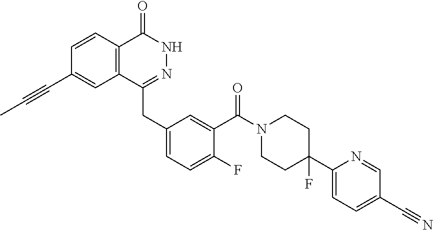 [SMILES: CC#Cc1ccc2c(=O)[nH]nc(Cc3ccc(F)c(C(=O)N4CCC(F)(c5ccc(C#N)cn5)CC4)c3)c2c1]

### 6-(4-Fluoro-1-(2-fluoro-5-((4-oxo-7-(prop-1-yn-1-yl)-3,4-dihydrophthalazin-1-yl)methyl)benzoyl)piperidin-4-yl)nicotinonitrile

To a solution of 6-(4-fluoropiperidin-4-yl)nicotinonitrile (75 mg, 0.31 mmol) and 2-fluoro-5-((4-oxo-7-(prop-1-yn-1-yl)-3,4-dihydrophthalazin-1-yl)methyl)benzoic acid (167 mg, 0.40 mmol) in DMF (2 mL) was added EDCI (77 mg, 0.50 mmol), HOBt (67 mg, 0.50 mmol) and DIEPA (214 mg, 1.66 mmol), The reaction mixture was stirred at rt for 2 hours. The reaction was purified by prep-HPLC to give 6-(4-fluoro-1-(2-fluoro-5-((4-oxo-7-(prop-1-yn-1-yl)-3,4-dihydrophthalazin-1-yl)methyl) benzoyl)piperidin-4-yl)nicotinonitrile as a white solid (65 mg, 37% yield). LCMS ESI m/z: 524 [M+H]⁺. ¹H NMR (400 MHz, CDCl₃) δ 10.13 (s, 1H), 8.85 (d, J=1.0 Hz, 1H), 8.35 (d, 1H), 8.03 (dd, J=8.3, 2.1 Hz, 1H), 7.77-7.66 (m, 3H), 7.37 (dd, J=6.1 Hz, 1H), 7.33-7.27 (m, 1H), 7.07 (t, J=8.8 Hz, 1H), 4.82 (dd, J=13.6 Hz, 1H), 4.25 (s, 2H), 3.56 (t, 2H), 3.21 (t, J=12.7 Hz, 1H), 2.56-2.16 (m, 2H), 2.09 (s, 3H), 2.03-1.93 (m, 1H), 1.86-1.73 (m, 1H).

### Synthesis of Example 68: 6-((1S,4S)-5-(2-fluoro-5-((4-oxo-7-(prop-1-ynyl)-3,4-dihydrophthalazin-1-yl)methyl)benzoyl)-2,5-diazabicyclo[2.2.1]heptan-2-yl)nicotinonitrile

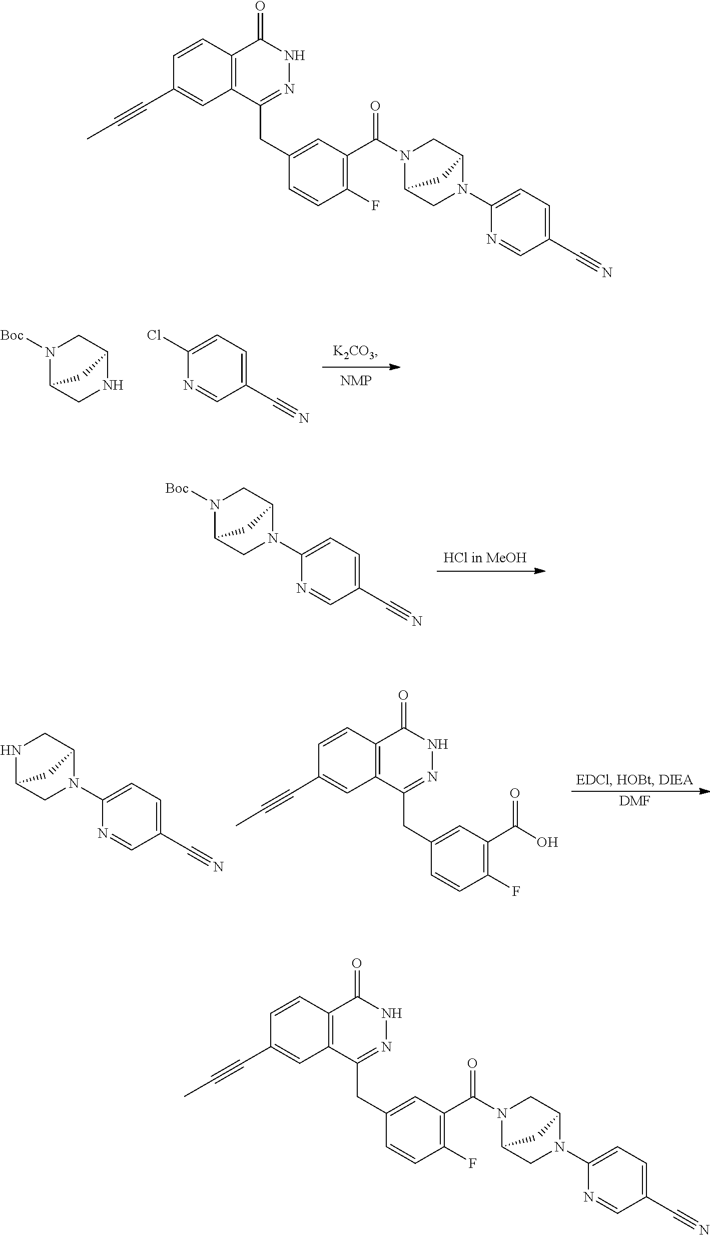

### Step 1

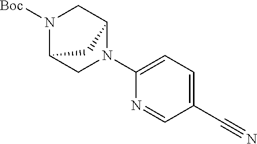 [SMILES: CC(C)(C)OC(=O)N1C[C@@H]2C[C@H]1CN2c1ccc(C#N)cn1]

### tert-Butyl(S,4S)-5-(5-cyano-2-pyridyl)-2,5-diazabicyclo[2.2.1]heptane-2-carboxylate

A mixture of tert-butyl (1S,4S)-2,5-diazabicyclo[2.2.1]heptane-2-carboxylate (200.00 mg, 1.01 mmol), 6-chloropyridine-3-carbonitrile (181.70 mg, 1.31 mmol) and K₂CO₃ (278.42 mg, 2.02 mmol) in NMP (8 mL) was stirred at room temperature. The mixture was stirred for 3 hours at 80° C. under nitrogen atmosphere. The mixture was dissolved in EtOAc (20 mL), washed with water (20 mL×3). The organic phase was dried over Na₂SO₄, concentrated under reduced pressure. The residue was purified by Prep-TLC (petroleum ether:EtOAc=1:1) to afford tert-butyl (1S,4S)-5-(5-cyano-2-pyridyl)-2,5-diazabicyclo[2.2.1]heptane-2-carboxylate (270 mg, 89% yield) as a colorless semi-solid. LCMS ESI m/z 245.1 [M-56]⁺.

### Step 2

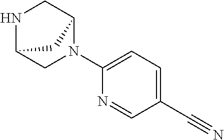 [SMILES: N#Cc1ccc(N2C[C@@H]3C[C@H]2CN3)nc1]

### 6-[(1S,4S)-2,5-Diazabicyclo[2.2.1]heptan-2-yl]pyridine-3-carbonitrile

A mixture of tert-butyl (1S,4S)-5-(5-cyano-2-pyridyl)-2,5-diazabicyclo[2.2.1]heptane-2-carboxylate (270 mg, 898.94 μmol) and HCl in MeOH (4 mol/L 5 mL) was stirred at rt for 1 hour. The reaction system was concentrated under reduced pressure to give 6-[(1S,4S)-2,5-diazabicyclo[2.2.1]heptan-2-yl]pyridine-3-carbonitrile (200 mg, crude) as a colorless oil. LCMS ESI m/z 201.1 [M+H]⁺.

### Step 3

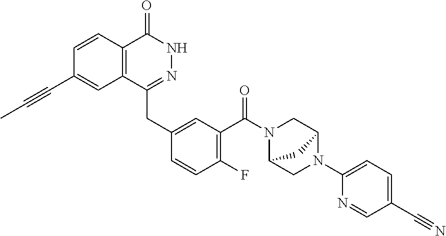 [SMILES: CC#Cc1ccc2c(=O)[nH]nc(Cc3ccc(F)c(C(=O)N4C[C@@H]5C[C@H]4CN5c4ccc(C#N)cn4)c3)c2c1]

### 6-[(1S,4S)-5-[2-Fluoro-5-[(4-oxo-7-prop-1-ynyl-3H-phthalazin-1-yl)methyl]benzoyl]-2,5-diazabicyclo[2.2.1]heptan-2-yl]pyridine-3-carbonitrile

A mixture of 2-fluoro-5-[(4-oxo-7-prop-1-ynyl-3H-phthalazin-1-yl)methyl]benzoic acid (50 mg. 148.67 μmol), 6-[(1S,4S)-2,5-diazabicyclo[2.2.1]heptan-2-yl]pyridine-3-carbonitrile (35.72 mg, 178.40 μmol), EDCI (42.59 mg, 223.00 μmol), HOBt (30.13 mg, 223.00 μmol) and DIPEA (57.54 mg, 446.01 μmol) in DMF (5 mL) was stirred for 3 hours at room temperature under nitrogen atmosphere. The solution was poured into water (10 mL), extracted with EtOAc (10 mL×3). The organic layers were dried over Na₂SO₄, concentrated under reduced pressure. The residue was purified by Prep-TLC (DCM:MeOH=10:1) to give 6-[(1S,4S)-5-[2-fluoro-5-[(4-oxo-7-prop-1-ynyl-3H-phthalazin-1-yl)methyl]benzoyl]-2,5-diazabicyclo[2.2.1]heptan-2-yl]pyridine-3-carbonitrile (7.0 mg, 9% yield) as a white solid. LCMS ESI m/z 519.2 [M+H]⁺. ¹H NMR (400 MHz, DMSO-d₆) δ 12.63 (s, 1H), 8.52-8.39 (m, 1H), 8.20 (dd, J=12.7, 8.2 Hz, 1H), 7.94-7.74 (m, 3H), 7.49-7.31 (m, 2H), 7.31-7.14 (m, 1H), 6.63 (s, 1H), 4.98 (s, 1H), 4.32 (dd, J=27.3, 4.2 Hz, 2H), 4.18 (s, 1H), 3.84-3.54 (m, 2H), 3.43 (dd, J=14.7, 9.9 Hz, 2H), 2.11 (d, J=5.4 Hz, 3H), 1.97 (dd, J=27.2, 11.0 Hz, 2H).

### Synthesis of Example 69: 2-((1-(2-fluoro-5-((4-oxo-7-(prop-1-yn-1-yl)-3,4-dihydrophthalazin-1-yl)methyl)benzoyl)azetidin-3-yl)(methyl)amino) isonicotinonitrile

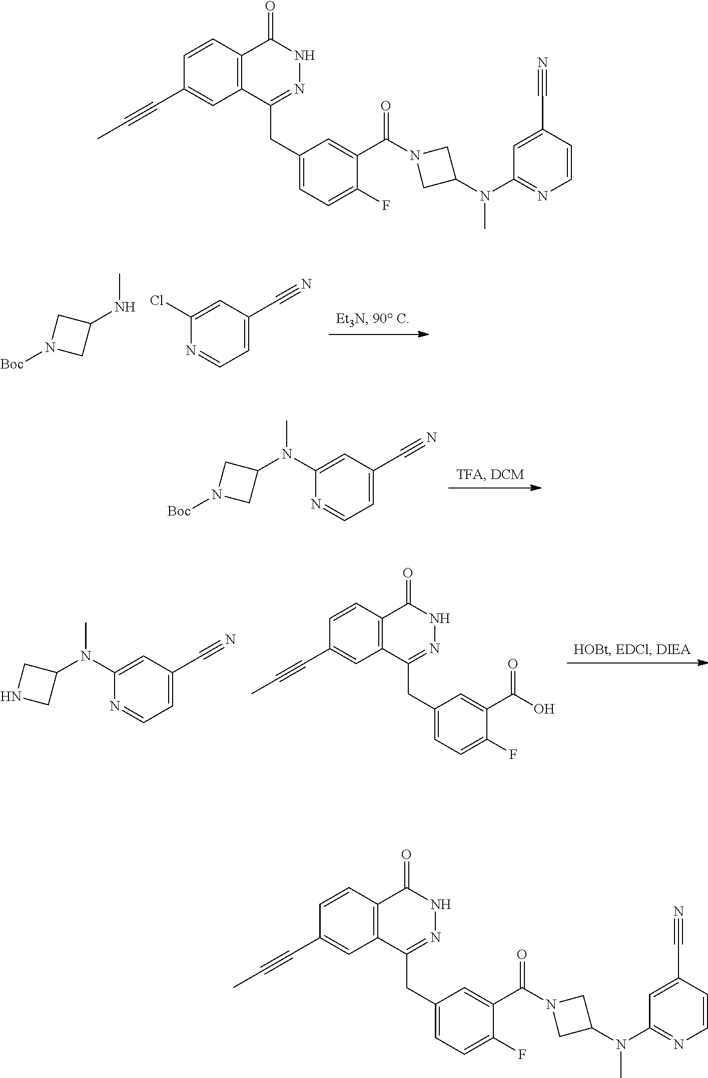

### Step 1

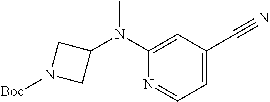 [SMILES: CN(c1cc(C#N)ccn1)C1CN(C(=O)OC(C)(C)C)C1]

### tert-Butyl 3-((4-cyanopyridin-2-yl)(methyl)amino)azetidine-1-carboxylate

A mixture of 2-chloroisonicotinonitrile (100 mg, 0.72 mmol), tert-butyl 3-(methylamino)azetidine-1-carboxylate (400 mg, 2.17 mmol) and Et₃N (150 mg, 1.44 mmol) was stirred at 90° C. for 16 hours. The mixture was cooled to rt, diluted with H₂O (5 mL) and extracted with EtOAc (10 mL×3). The combined organic layer was washed with brine, dried over anhydrous Na₂SO₄, filtered and concentrated in vacuo. The residue was purified by silica gel chromatography (EtOAc/pet ether=40 to 60%) to afford tert-butyl 3-((4-cyanopyridin-2-yl)(methyl)amino)azetidine-1-carboxylate as a colorless oil (110 mg, 53% yield). LCMS ESI m/z 289 [M+H]⁺.

### Step 2

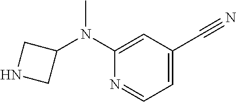

### 2-(Azetidin-3-yl(methyl)amino)isonicotinonitrile

To a solution of tert-butyl 3-((4-cyanopyridin-2-yl)(methyl)amino)azetidine-1-carboxylate (0.11 g, 0.38 mmol) in DCM (5 mL) was added TFA (0.5 mL). The mixture was stirred at rt for 1 hour. The solution was concentrated in vacuo to give 2-(azetidin-3-yl(methyl)amino)isonicotinonitrile as a colorless oil (70 mg, 97% yield). This was used without further purification. LCMS ESI m/z 189 [M+H]1.

### Step 3

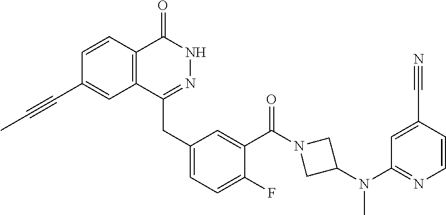 [SMILES: CC#Cc1ccc2c(=O)[nH]nc(Cc3ccc(F)c(C(=O)N4CC(N(C)c5cc(C#N)ccn5)C4)c3)c2c1]

### 2-((1-(2-Fluoro-5-((4-oxo-7-(prop-1-yn-1-yl)-3,4-dihydrophthalazin-1-yl)methyl)benzoyl)azetidin-3-yl)methyl)amino)isonicotinonitrile

To a solution of 2-(azetidin-3-yl(methyl)amino)isonicotinonitrile (70 mg, 0.40 mmol) and 2-fluoro-5-((4-oxo-7-(prop-1-yn-1-yl)-3,4-dihydrophthalazin-1-yl)methyl)benzoic acid (147 mg, 0.44 mmol) in DMF (5 mL) was added EDCI (115 mg, 0.60 mmol), HOBt (81 mg, 0.60 mmol) and DIPEA (154 mg, 1.20 mmol), The mixture was stirred at rt for 16 hours. The reaction was purified by prep-HPLC to give 2-((1-(2-fluoro-5-((4-oxo-7-(prop-1-yn-1-yl)-3,4-dihydrophthalazin-1-yl)methyl)benzoyl)azetidin-3-yl)(methyl)amino)isonicotinonitrile as a white solid (10 mg, 4% yield). LCMS ESI 507 (M+H)⁺; ¹H NMR (400 MHz, DMSO-d₆) δ 12.63 (s, 1H), 8.61 (t, J=5.9 Hz, 1H), 8.43 (d, J=6.7 Hz, 1H), 8.20 (d, J=8.2 Hz, 1H), 7.95 (s, 1H), 7.90 (s, 1H), 7.77 (dd, J=8.2, 1.4 Hz, 1H), 7.35 (dd, J=6.7, 2.2 Hz, 1H), 7.25 (dd, J=6.6, 1.4 Hz, 1H), 7.19 (dd, J=10.1, 8.6 Hz, 1H), 4.82 (dd, J=13.2, 11.4 Hz, 1H), 4.56 (dd, J=13.5, 6.2 Hz, 1H), 4.51-4.42 (m, 1H), 3.80-3.69 (m, 2H), 3.68-3.60 (m, 2H), 3.16 (s, 3H), 2.10 (s, 3H).

### Synthesis of Example 70: 6-((1-(2-Fluoro-5-((4-oxo-7-(prop-1-yn-1-yl)-3,4-dihydrophthalazin-1-yl)methyl)benzoyl)azetidin-3-yl)(methyl)amino)nicotinonitrile 2,2,2-trifluoroacetate

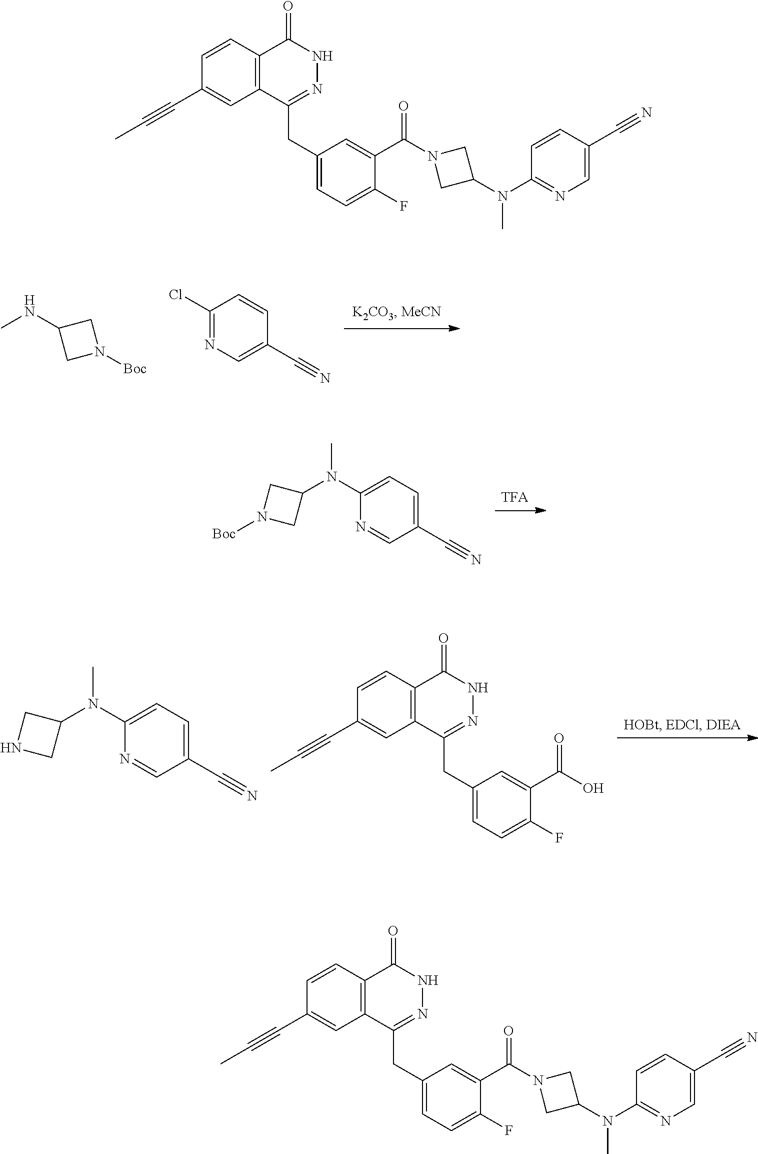

### Step 1

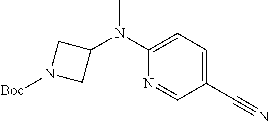 [SMILES: CN(c1ccc(C#N)cn1)C1CN(C(=O)OC(C)(C)C)C1]

### tert-Butyl 3-((5-cyanopyridin-2-yl)methyl)amino)azetidine-1-carboxylate

A mixture of 6-chloropyridine-3-carbonitrile (500 mg, 3.61 mmol), tert-butyl N-[2-(ethylamino)ethyl]carbamate (672 mg, 3.61 mmol) and K₂CO₃ (1.25 g, 9.02 mmol) in MeCN (20 mL) was stirred at 60° C. for 16 hours. The reaction was concentrated in vacuo, diluted with water (30 mL) and extracted with EtOAc (30 mL×3). The combined organic layer was washed with brine, dried over anhydrous Na₂SO₄, filtered and concentrated in vacuo. The residue was purified by silica gel chromatography (EtOAc/pet ether=10 to 20%) to give tert-butyl 3-((5-cyanopyridin-2-yl)(methyl)amino)azetidine-1-carboxylate as a white solid (1.2 g, 70/o yield). LCMS ESI m/z 291 [M+H]⁺.

### Step 2

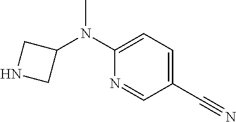

### 6-(Azetidin-3-yl(methyl)amino)nicotinonitrile

A solution of tert-butyl 3-((5-cyanopyridin-2-yl)(methyl)amino)azetidine-1-carboxylate (200 mg, 0.69 mmol) in DCM (5 mL) and TFA (0.5 mL) was stirred at rt for 1 hour. The solvent was removed in vacuo to give 6-(azetidin-3-yl(methyl)amino)nicotinonitrile as a yellow oil (129 mg, quant.). This was used without further purification. LCMS ESI m/z 189 [M+H]⁺.

### Step 3

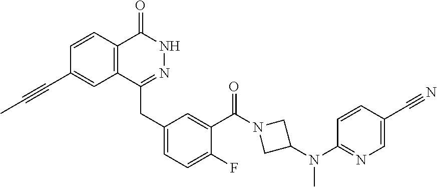 [SMILES: CC#Cc1ccc2c(=O)[nH]nc(Cc3ccc(F)c(C(=O)N4CC(N(C)c5ccc(C#N)cn5)C4)c3)c2c1]

### 6-((1-(2-Fluoro-5-((4-oxo-7-(prop-1-yn-1-yl)-3,4-dihydrophthalazin-1-yl)methyl)benzoyl)azetidin-3-yl)(methyl)amino)nicotinonitrile 2,2,2-trifluoroacetate

To a solution of 6-(azetidin-3-yl(methyl)amino)nicotinonitrile (80 mg, 0.13 mmol) and 2-fluoro-54(4-oxo-7-(prop-1-yn-1-yl)-3,4-dihydrophthalazin-1-yl)methyl)benzoic acid (50 mg, 0.13 mmol) in DMF (2 mL) was added EDCI (38 mg, 0.2 mmol), HOBt (27 mg, 0.2 mmol) and DIPEA (88 mg, 0.67 mmol), The reaction mixture was stirred at rt for 2 hours. The reaction was purified by prep-HPLC to give 6-((1-(2-fluoro-5-((4-oxo-7-(prop-1-yn-1-yl)-3,4-dihydrophthalazin-1-yl)methyl)benzoyl)azetidin-3-yl)(methyl)amino)nicotinonitrile 2,2,2-trifluoroacetate as a white solid (17.5 mg, 21% yield). LCMS ESI m/z 507 (M+H)⁺; ¹H NMR (400 MHz, DMSO-d₆) δ 12.63 (s, 1H), 9.03 (s, 1H), 8.62 (s, 1H), 8.30 (dd, J=9.4, 1.7 Hz, 1H), 8.20 (d, J=8.2 Hz, 1H), 7.92 (s, 1H), 7.78 (d, J=8.3 Hz, 1H), 7.47 (s, 1H), 7.35 (d, J=9.3 Hz, 2H), 7.26-7.15 (m, 1H), 4.79 (t, J=11.4 Hz, 1H), 4.64-4.45 (m, 2H), 4.30 (s, 2H), 3.79 (d, J=10.8 Hz, 1H), 3.59 (d, J=14.2 Hz, 1H), 3.22 (s, 3H), 2.11 (s, 3H).

### Synthesis of Example 71: 6-(4-(2-Fluoro-5-((4-oxo-7-(prop-1-yn-1-yl)-3,4-dihydrophthalazin-1-yl)methyl)benzoyl)-3,3-dimethyl-2-oxopiperazin-1-yl)nicotinonitrile

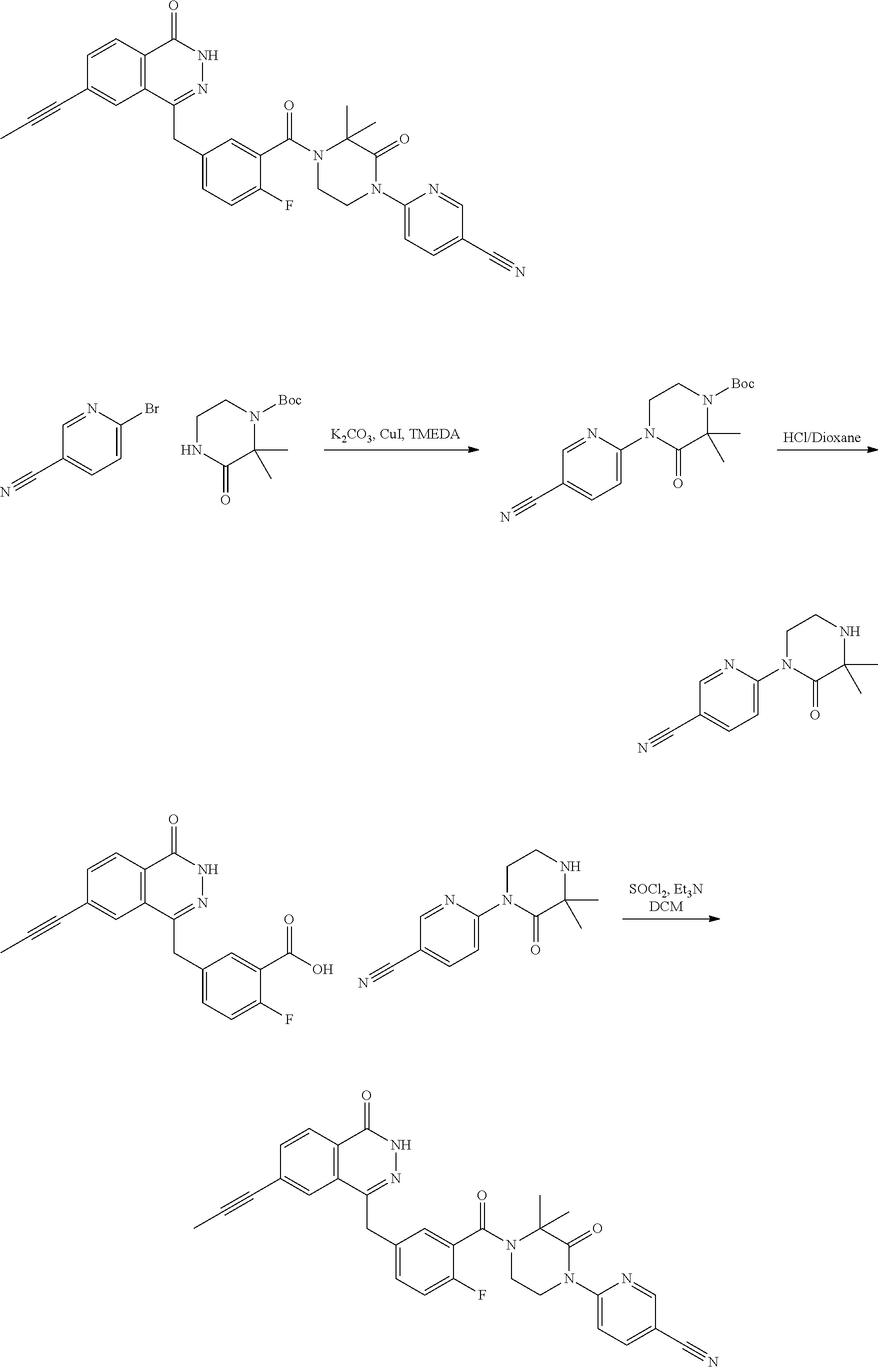

### Step 1

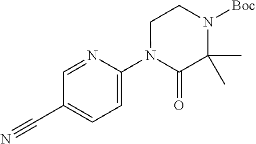 [SMILES: CC(C)(C)OC(=O)N1CCN(c2ccc(C#N)cn2)C(=O)C1(C)C]

### tert-Butyl 4-(5-cyanopyridin-2-yl)-2,2-dimethyl-3-oxopiperazine-1-carboxylate

A mixture of tert-butyl 2,2-dimethyl-3-oxopiperazine-1-carboxylate (600 mg, 2.63 mmol), 6-bromonicotinonitrile (481 mg, 2.63 mmol), K₂CO₃ (726 mg, 5.26 mmol), CuI (5 mg, 0.03 mmol) and TMEDA (31 mg, 0.26 mmol) in 1,4-dioxane (3 mL) was stirred at 120° C. under Ar (g) for 16 h. The solids were removed by filtration, and the filtrate was concentrated in vacuo. The residue was purified by silica gel chromatography (EtOAc/petroleum ether=0 to 9%) to give tert-butyl 4-(5-cyanopyridin-2-yl)-2,2-dimethyl-3-oxopiperazine-1-carboxylate (230 mg, 26% yield) as a white solid. LCMS ESI m/z 331 [M+H]⁺.

### Step 2

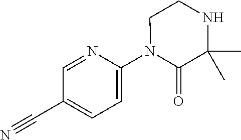 [SMILES: CC1(C)NCCN(c2ccc(C#N)cn2)C1=O]

### 6-(3,3-Dimethyl-2-oxopiperazin-1-yl)nicotinonitrile

A solution of tert-butyl 4-(5-cyanopyridin-2-yl)-2,2-dimethyl-3-oxopiperazine-1-carboxylate (230 mg, 0.70 mmol) in HCl/dioxane (4M, 5 mL) was stirred at 25° C. for 1 hour. The reaction mixture was concentrated in vacuo to give 6-(3,3-dimethyl-2-oxopiperazin-1-yl)nicotinonitrile as a white solid (180 mg, 97% yield). This was used without further purification. LCMS ESI m/z 231 [M+H]⁺.

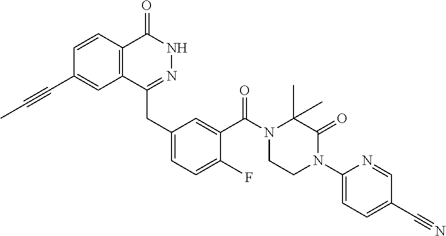 [SMILES: CC#Cc1ccc2c(=O)[nH]nc(Cc3ccc(F)c(C(=O)N4CCN(c5ccc(C#N)cn5)C(=O)C4(C)C)c3)c2c1]

### 6-(4-(2-Fluoro-5-((4-oxo-7-(prop-1-yn-1-yl)-3,4-dihydrophthalazin-1-yl)methyl)benzoyl)-3,3-dimethyl-2-oxopiperazin-1-yl)nicotinonitrile

A solution of 2-fluoro-5-((4-oxo-7-(prop-1-yn-1-yl)-3,4-dihydrophthalazin-1-yl)methyl) benzoic acid (304 mg, 0.90 mmol) in SOCl₂ (1.95 g, 16.42 mmol) was stirred at 25° C. under Ar (g) for 3 hours. The mixture was concentrated in vacuo to obtain a white solid. The obtained solid was dissolved in DCM (20 mL), then a solution of 6-(3,3-dimethyl-2-oxopiperazin-1-yl)nicotinonitrile (180 mg, 0.82 mmol) and Et₃N (249 mg, 2.46 mmol) in DCM (10 mL) was added. The mixture was stirred at rt under Ar (g) for 4 hours, then diluted with H₂O (20 mL) and extracted with DCM (30 mL×3). The combined organic layer was dried over anhydrous Na₂SO₄, filtered and concentrated in vacuo. The residue was purified by silica gel chromatography (EtOAc/petroleum ether=0 to 50%) to afford 6-(4-(2-fluoro-5-((4-oxo-7-(prop-1-yn-1-yl)-3,4-dihydrophthalazin-1-yl)methyl)benzoyl)-3,3-dimethyl-2-oxopiperazin-1-yl)nicotinonitrile as a white solid (174 mg, 39% yield). LCMS ESI 549 (M+H)⁺; ¹H NMR (40) MHz, DMSO-d₆) δ 12.64 (s, 1H), 8.93 (dd, J=2.3, 0.8 Hz, 1H), 8.31 (dd, J=8.9, 2.3 Hz, 1H), 8.22-8.20 (m, 1H), 8.20-8.18 (m, 1H), 7.95 (s, 1H), 7.78 (dd, J=8.2, 1.4 Hz, 1H), 7.45 (dd, J=6.5, 2.1 Hz, 1H), 7.42-7.37 (m, 1H), 7.29-7.22 (m, 1H), 4.34 (s, 2H), 4.06-4.00 (m, 2H), 3.58 (s, 2H), 2.08 (d, J=2.1 Hz, 3H), 1.83 (s, 6H).

### Synthesis of Example 72: 6-(4-(2-Fluoro-5-((4-oxo-7-(prop-1-ynyl)-3,4-dihydrophthalazin-1-yl)methyl)benzoyl)-2-methylpiperazin-1-yl)nicotinonitrile

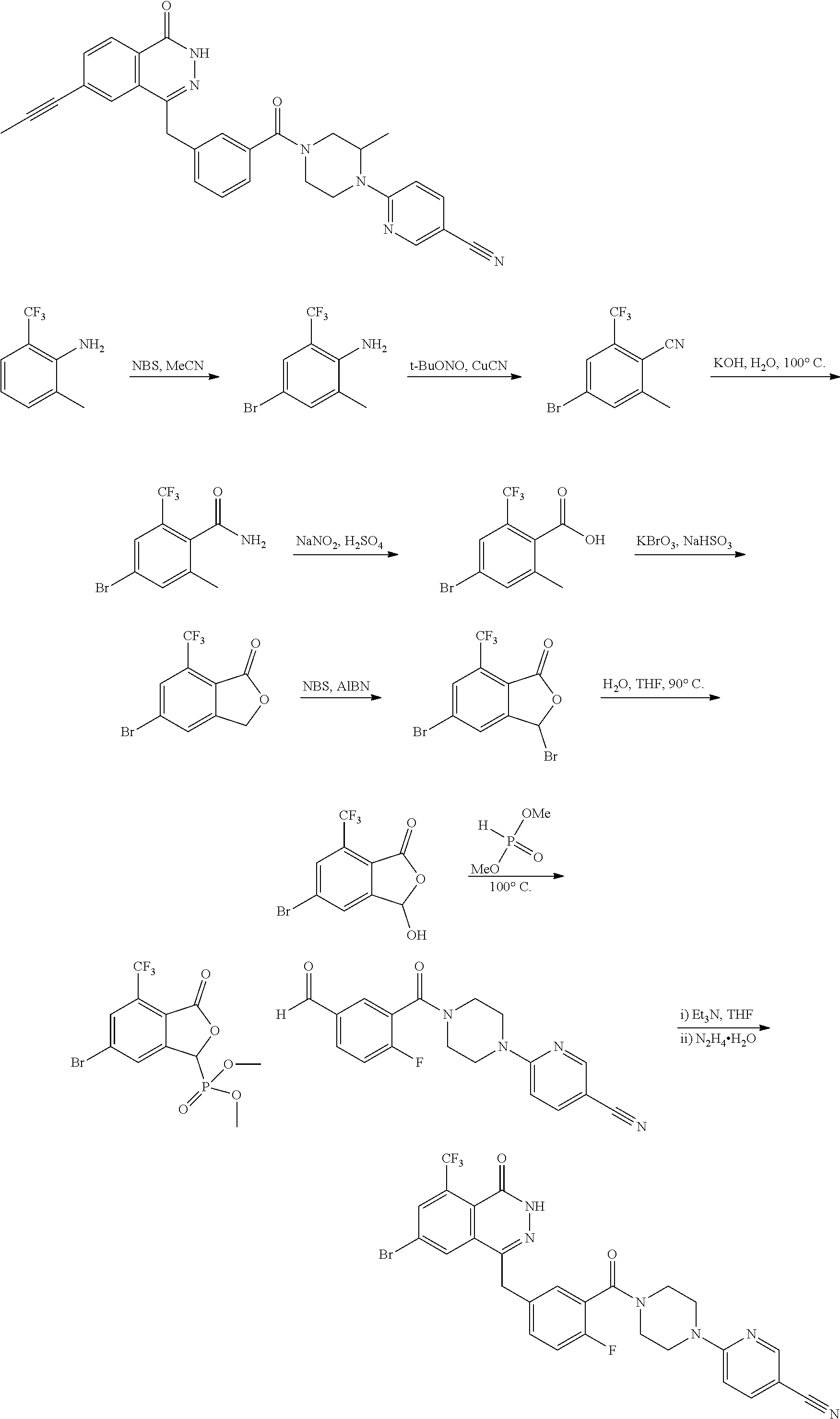

### Step 1

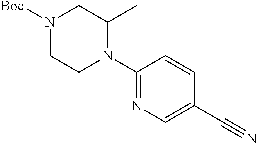 [SMILES: CC1CN(C(=O)OC(C)(C)C)CCN1c1ccc(C#N)cn1]

### tert-Buty 4-(5-cyanopyridin-2-yl)-3-methylpiperazine-1-carboxylate

To a solution of 6-chloropyridine-3-carbonitrile (1.5 g, 10.83 mmol) in NMP (15 mL) was added tert-butyl 3-methylpiperazine-1-carboxylate (2.17 g, 10.83 mmol) and K₂CO₃ (2.99 g, 21.65 mmol), The reaction mixture was stirred at 80° C. under N₂(g) for 18 hours. The reaction mixture was worked up with water (20 mL) and EtOAc (30 mL). The organic layer was washed with brine, dried over Na₂SO₄ and concentrated to give a residue. The residue was purified by silica gel chromatography (petroleum ether/EtOAc=20/1 to 4/1) to give tert-butyl 4-(5-cyano-2-pyridyl)-3-methy-piperazine-1-carboxylate (1.8 g, 55% yield) as a white solid. ¹H NMR (400 MHz, CDCl₃) δ 8.42 (d, J=2.0 Hz, 1H), 7.63 (dd, J=9.0, 2.3 Hz, 1H) 6.56 (d, J=9.0 Hz, 1H), 4.53 (s, 1H), 4.12 (dd, J=14.3, 7.1 Hz 2H) 3.98 (d, J=24.8 Hz, 1H), 3.31-3.12 (m, 2H), 3.02 (s, 1H), 1.49 (s, 9H), 1.20 (d, J=6.7 Hz, 3H).

### Step 2

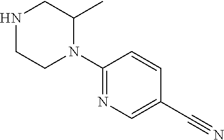

### 6-(2-Methyl-4-piperazin-1-yl)nicotinonitrile

A solution of tert-butyl 4-(5-cyano-2-pyridyl)-3-methyl-piperazine-1-carboxylate (1.8 g, 5.95 mmol) in EtOAc (5 mL) was added HCl/EA (12 mL, 4 M). The reaction mixture was stirred at rt for 3 hours. The mixture was concentrated to give 6-(2-methylpiperazin-1-yl)pyridine-3-carbonitrile (1.4 g, crude). LCMS ESI m/z 203.2 [M+H]⁺.

### Step 3

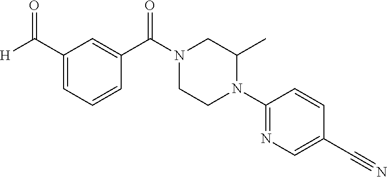 [SMILES: CC1CN(C(=O)c2cccc(C=O)c2)CCN1c1ccc(C#N)cn1]

### 6-(4-(2-Fluoro-5-formylbenzoyl)-2-methylpiperazin-1-yl)nicotinonitrile

To a solution of 2-fluoro-5-formyl-benzoic acid (352.14 mg, 2.09 mmol) in DMF (8 mL) was added HATU (955.69 mg, 2.51 mmol) and Et₃N (423.89 mg, 4.19 mmol, 583.87 μL). The reaction mixture was stirred at rt for 15 min. 6-(2-Methylpiperazin-1-yl)pyridine-3-carbonitrile (500 mg, 2.09 mmol) was added to the mixture and the reaction mixture was stirred at rt for another 8 hours. The mixture was poured into water (20 mL), extracted with EtOAc (20 mL×3). The organic layer was concentrated to the crude. The crude was purified by silica gel chromatography (petroleum ether/EtOAc=2/1) to give 6-[4-(2-fluoro-5-formyl-benzoyl)-2-methyl-piperazin-1-yl]pyridine-3-carbonitrile (550 mg, 75% yield) as a white solid. LCMS ESI m/z 353.1 [M+H]⁺.

### Step 4

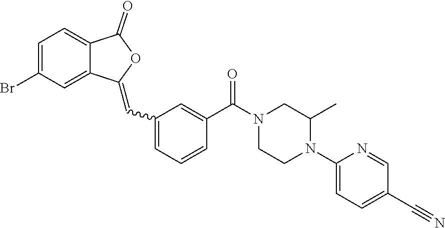 [SMILES: CC1CN(C(=O)c2cccc(C=C3OC(=O)c4ccc(Br)cc43)c2)CCN1c1ccc(C#N)cn1]

### 6-(4-(5-((6-Bromo-3-oxoisobenzofuran-1(3H)-ylidene)methyl)-2-fluorobenzoyl)-2-methylpiperazin-1-yl)nicotinonitrile

To a solution of 5-bromo-3-dimethoxyphosphoryl-3H-isobenzofuran-1-one (210 mg, 654.08 μmol) in THF (10 mL) was added 6-[4-(2-fluoro-5-formyl-benzoyl)-2-methyl-piperazin-1-yl]pyridine-3-carbonitrile (276.57 mg, 784.90 μmol) and Et₃N (198.56 mg, 1.96 mmol, 273.50 μL). The reaction mixture was stirred at rt for 16 hours. The mixture was poured into sat. NaHSO₃, extracted with DCM (30 mL×3), dried over Na₂SO₄. The organic layer was concentrated to give 6-[4-[5-[(6-bromo-3-oxo-isobenzofuran-1-ylidene)methyl]-2-fluoro-benzoyl]-2-methyl-piperazin-1-yl]pyridine-3-carbonitrile (330 mg, crude) as a light yellow solid. LCMS ESI m/z 549.6 [M+H]⁺.

### Step 5

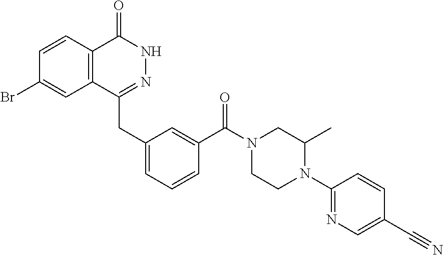 [SMILES: CC1CN(C(=O)c2cccc(Cc3n[nH]c(=O)c4ccc(Br)cc34)c2)CCN1c1ccc(C#N)cn1]

### 6-(4-(5-((7-Bromo-4-oxo-3,4-dihydrophthalazin-1-yl)methyl)-2-fluorobenzoyl)-2-methylpiperazin-1-yl)nicotinonitrile

A solution of 6-[4-[5-[(Z)-(6-bromo-3-oxo-isobenzofuran-1-ylidene)methyl]-2-fluoro-benzoyl]-2-methyl-piperazin-1-yl]pyridine-3-carbonitrile (330 mg, 602.88 μmol) and N₂H₄·H₂O (602.88 μmol, 2 mL, 80% purity) in THF (5 mL) was heated to 80° C. for 3 hours. The mixture was poured into water (20 mL), extracted with DCM (20 mL×3). The extracts was dried over anhydrous Na₂SO₄, filtered and concentrated in vacuo. The residue was purified by silica gel chromatography to give 6-[4-[5-[(7-bromo-4-oxo-3H-phthalazin-1-yl)methyl]-2-fluoro-benzoyl]-2-methyl-piperazin-1-yl]pyridine-3-carbonitrile (230 mg, 68% yield) as a light yellow gum. LCMS ESI m/z 561.1 [M+H]⁺.

### Step 6

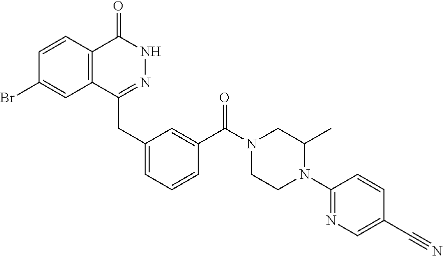 [SMILES: CC1CN(C(=O)c2cccc(Cc3n[nH]c(=O)c4ccc(Br)cc34)c2)CCN1c1ccc(C#N)cn1]

### 6-(4-(2-Fluoro-5-((4-oxo-7-(prop-1-yn-1-yl)-3,4-dihydrophthalazin-1-yl)methyl)benzoyl)-2-methylpiperazin-1-yl)nicotinonitrile

To a mixture of 6-[4-[5-[(7-bromo-4-oxo-3H-phthalazin-1-yl)methyl]-2-fluoro-benzoyl]-2-methyl-piperazin-1-yl]pyridine-3-carbonitrile (100 mg, 178.12 μmol) in toluene (2 mL) was added Pd(PPh₃)₄ (20.58 mg, 17.81 μmol) and tributyl(prop-1-ynyl)stannane (117.24 mg, 356.25 μmol). The reaction mixture was stirred at 100° C. under microwave for 1 hr. The mixture was purified by Prep-HPLC to give 6-[4-[2-fluoro-5-[(4-oxo-7-prop-1-ynyl-3H-phthalazin-1-yl)methyl]benzoyl]-2-methyl-piperazin-1-yl]pyridine-3-carbonitrile (24.57 mg, 27% yield) as a white solid: LCMS ESI 520.8 [M+H]⁺; ¹H NMR (400 MHz, DMSO-d₆) δ 12.63 (s, 1H), 8.52 (t, J=2.5 Hz, 1H), 8.20 (dd, J=8.2, 1.0 Hz, 1H), 7.90 (ddd. J=15.0, 11.3, 7.4 Hz, 2H), 7.77 (d, J=8.1 Hz, 1H), 7.41 (ddd. J=23.5, 13.5, 5.2 Hz, 2H), 7.26 (t, J=9.0 Hz, 1H), 6.88 (dd, J=11.6, 9.3 Hz, 1H), 4.79 (s, 1H), 4.53 (s, 1H), 4.46-4.10 (m, 4H). 3.44 (d, J=13.1 Hz, 1H), 3.29-2.99 (m, 3H), 2.08 (d, J=4.8 Hz, 3H), 1.02 (dd, J=99.0, 5.5 Hz, 3H).

### Synthesis of Example 73: 6-(4-(2-Fluoro-5-((4-oxo-7-(prop-1-yn-1-yl)-3,4-dihydrophthalazin-1-yl)methyl-d2)benzoyl)piperazin-1-yl)nicotinonitrile

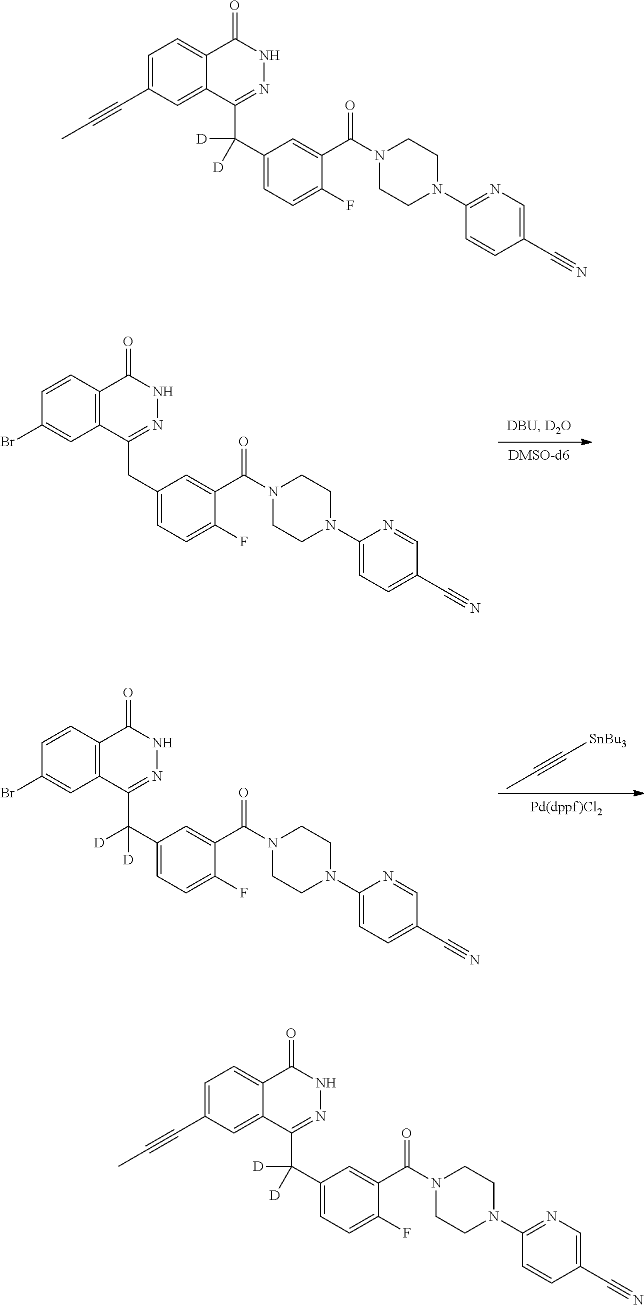

### Step 1:

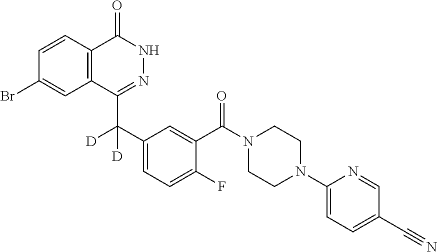 [SMILES: [2H]C([2H])(c1ccc(F)c(C(=O)N2CCN(c3ccc(C#N)cn3)CC2)c1)c1n[nH]c(=O)c2ccc(Br)cc12]

### 6-(4-(5-((7-Bromo-4-oxo-3,4-dihydrophthalazin-1-yl)methyl-d2)-2-fluorobenzoyl)piperazin-1-yl)nicotinonitrile

A solution of 6-[4-[5-[(7-bromo-4-oxo-3H-phthalazin-1-yl)methyl]-2-fluoro-benzoyl]piperazin-1-yl]pyridine-3-carbonitrile (100 mg, 0.18 mmol) and DBU (56 mg, 0.37 mmol) in dry DMSO-de (15 mL) and DD) (5 mL) were stirred at 70° C. under Ar (g) for 18 hours. The reaction was cooled to rt, diluted with H₂O (20 mL) and filtered. The obtained solids were dried under vacuum to obtain 6-[4-[5-[(7-bromo-4-oxo-3H-phthalazin-1-yl)-dideuterio-methyl]-2-fluoro-benzoyl]piperazin-1-yl]pyridine-3-carbonitrile as a white solid (88 mg, 88% yield). This was used without further purification. LCMS ESI m/z: 549 [M+H]⁺.

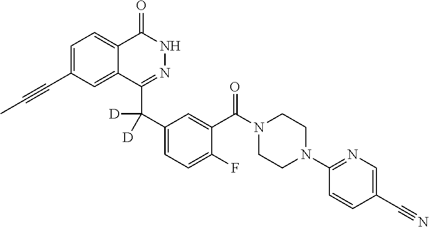 [SMILES: [2H]C([2H])(c1ccc(F)c(C(=O)N2CCN(c3ccc(C#N)cn3)CC2)c1)c1n[nH]c(=O)c2ccc(C#CC)cc12]

### 6-(4-(2-Fluoro-5-((4-oxo-7-(prop-1-yn-1-yl)-3,4-dihydrophthalazin-1-yl)methyl-d2)benzoyl)piperazin-1-yl)nicotinonitrile

A mixture of 6-[4-[5-[(7-bromo-4-oxo-3H-phthalazin-1-yl)-dideuterio-methyl]-2-fluoro-benzoyl]piperazin-1-yl]pyridine-3-carbonitrile (88 mg, 0.16 mmol), tributyl (prop-1-ynyl)stannane (160 mg, 0.48 mmol) and Pd(dppf)Cl₂ (15 mg, 0.02 mmol) in 1,4-dioxane (5 mL) was stirred at 100° C. under Ar (g) for 6 hours. The reaction mixture was concentrated in vacuo and the residue was purified by prep-HPLC to afford 6-(4-(2-fluoro-5-((4-oxo-7-(prop-1-yn-1-yl)-3,4-dihydrophthalazin-1-yl)methyl-d2)benzoyl)piperazin-1-yl)nicotinonitrile (31 mg, 37% yield) as a white solid. LCMS ESI m/z 509 [M+H]⁺. ¹H NMR (400 MHz, DMSO-d₆) δ 12.64 (s, 1H), 8.51 (d, J=2.0 Hz, 1H), 8.19 (d, J=8.2 Hz, 1H), 7.94 (s, 1H), 7.90 (dd, J=9.1, 2.3 Hz, 1H), 7.78 (dd, J=8.2, 1.4 Hz, 1H), 7.47-7.37 (m, 2H), 7.26 (t, J=9.1 Hz, H), 6.93 (d, J=9.1 Hz, 1H), 3.75 (dd, J=13.7, 3.6 Hz, 4H), 3.62 (t, J=4.7 Hz, 2H), 3.35-3.25 (m, 2H), 2.08 (s, 3H).

### Synthesis of Example 74: 6-(4-(2,3-Difluoro-5-((4-oxo-7-(prop-1-ynyl)-3,4-dihydrophthalazin-1-yl)methyl)benzoyl)piperazin-1-yl)nicotinonitrile

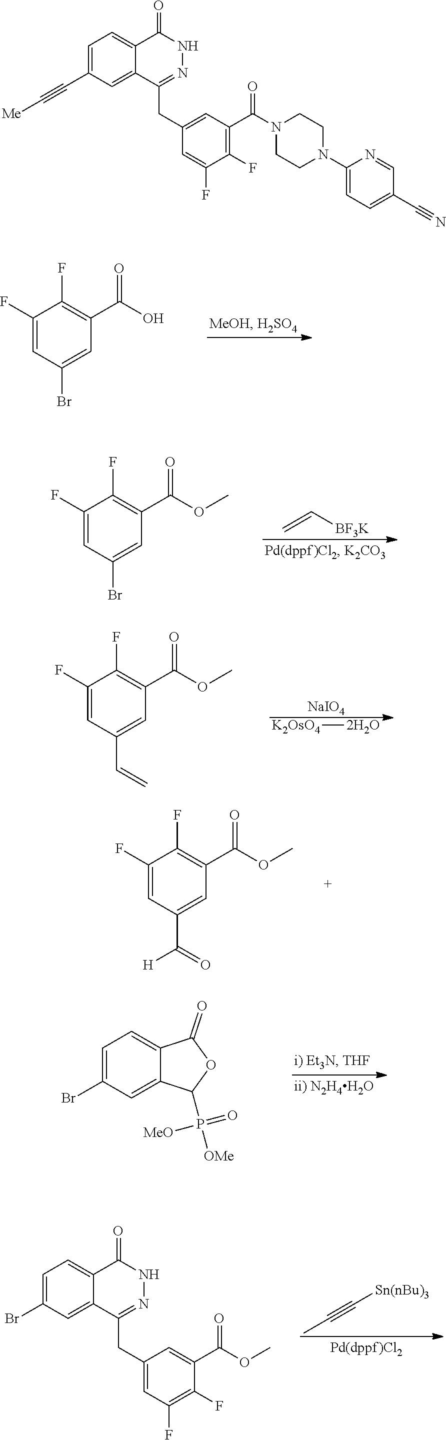

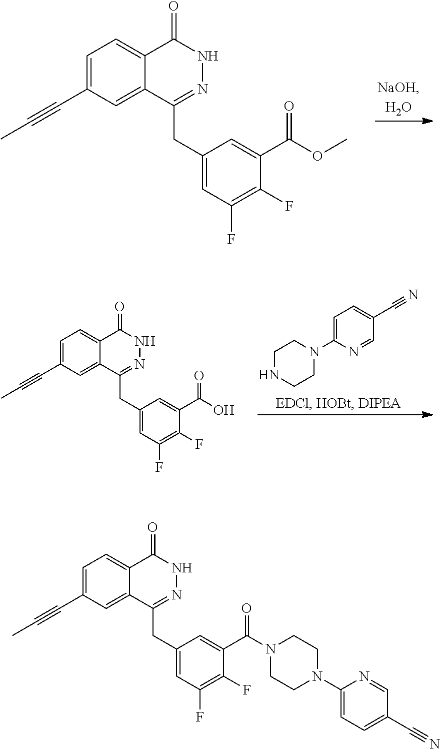

### Step 1

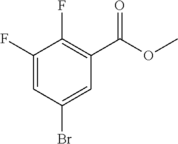

### Methyl 5-bromo-2,3-difluorobenzoate

To a solution of 5-bromo-2,3-difluoro-benzoic acid (1.1 g, 4.64 mmol) in MeOH (25 mL) was added conc. H₂SO₄ (4.05 g, 41.27 mmol, 2.20 mL) at rt, then stirred at 70° C. for 2 hours. The solution was concentrated in vacuo. This residue was diluted with water (30 mL) and extracted with EtOAc (20 mL×3). The combined organic layer was washed with brine, dried over anhydrous Na₂SO₄, filtered and concentrated in vacuo. The residue was purified by silica gel chromatography (EtOAc/petroleum ether=0 to 4%) to afford methyl 5-bromo-2,3-difluoro-benzoate as a colorless oil (1.08 g, 93% yield). LCMS ESI m/z: 251 [M+H]1.

### Step 2

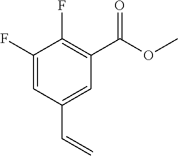

### Methyl 2,3-difluoro-5-vinylbenzoate

A mixture of methyl 5-bromo-2,3-difluoro-benzoate (0.98 g, 3.90 mmol), potassium vinyltrifluoroborate (1.05 g, 7.81 mmol), K₂CO₃ (2.16 g, 15.62 mmol) and Pd(dppf)Cl₂ (286 mg, 0.39 mmol) in MeCN (16 mL) and H₂O (4 mL) was stirred at 80° C. under Ar (g) for 7 hours. The mixture was cooled to rt, diluted with H₂O (5 mL) and extracted with EtOAc (10 mL×3). The combined organic layer was washed with brine, dried over anhydrous Na₂SO₄, filtered and concentrated in vacuo. The residue was purified by silica gel chromatography (EtOAc/petroleum ether=0 to 7%) to give methyl 2,3-difluoro-5-vinyl-benzoate as a colorless oil (0.7 g, 90% yield). LCMS ESI m/z: 199 [M+H]⁺. ¹H NMR (400 MHz, CDCl₃) δ 7.69-7.61 (m, 1H), 7.39-7.36 (m, 1H), 6.63-6.59 (m, 1H), 5.73 (d, J=16 Hz, 1H), 5.35 (d, J=12 Hz, 1H).

### Step 3

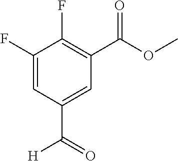

### Methyl 2,3-difluoro-5-formylbenzoate

To a mixture of methyl 2,3-difluoro-5-vinyl-benzoate (678 mg, 3.42 mmol) in THF (10 mL) and H₂O (10 mL) was added K₂OsO₄-2H₂O (13 mg, 0.34 mmol), The mixture was stirred at rt for 5 min, then NaIO₄ (2.20 g, 10.26 mmol) was added and the mixture was stirred at rt for 2 hours. The solids were removed by filtration. The filtrate was diluted with H₂O (5 mL) and extracted with EtOAc (10 mL×3). The combined organic layer was washed with brine, dried over anhydrous Na₂SO₄, filtered and concentrated in vacuo to give methyl 2,3-difluoro-5-formyl-benzoate as a colorless oil (0.68 g, 99% yield). This was used without further purification. LCMS ESI m/z: 201 [M+H]⁺.

### Step 4

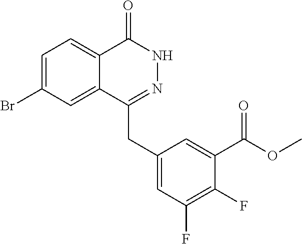 [SMILES: COC(=O)c1cc(Cc2n[nH]c(=O)c3ccc(Br)cc23)cc(F)c1F]

Methyl 5-((7-bromo-4-oxo-3,4-dihydrophthalazin-1-yl)methyl)-2,3-difluorobenzoate A solution of 5-bromo-3-dimethoxyphosphoryl-3H-isobenzofuran-1-one (176 mg, 0.55 mmol), methyl 2,3-difluoro-5-formyl-benzoate (100 mg, 0.50 mmol) and Et₃N (152 mg, 1.50 mmol) in anhydrous THF (5 mL) was stirred at 25° C. under Ar (g) for 15 hours. Hydrazine hydrate (50 mg, 1 mmol) was added and the reaction was stirred at 70° C. for 1 hour. The reaction was cooled to rt, then THF was removed under vacuum. The solids were washed with water (10 mL) and petroleum ether/EtOAc (2:1, 10 mL) to give methyl 5-[(7-bromo-4-oxo-3H-phthalazin-1-yl)methyl]-2,3-difluoro-benzoate as a white solid (130 mg, 64% yield). LCMS ESI m/z: 408.9 [M+H]⁺.

### Step 5

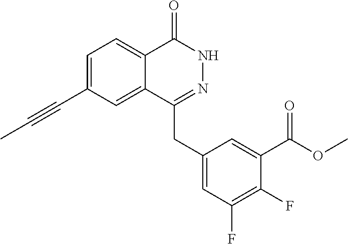 [SMILES: CC#Cc1ccc2c(=O)[nH]nc(Cc3cc(F)c(F)c(C(=O)OC)c3)c2c1]

### Methyl 2,3-difluoro-5-((4-oxo-7-(prop-1-ynyl)-3,4-dihydrophthalazin-1-yl)methyl)benzoate

A mixture of tributyl(prop-1-ynyl)stannane (230 mg, 0.70 mmol), methyl 5-[(7-bromo-4-oxo-3H-phthalazin-1-yl)methyl]-2,3-difluoro-benzoate (130 mg, 0.32 mmol) and Pd(dppf)Cl₂ (16 mg, 0.02 mmol) in 1,4-dioxane (5 mL) was stirred at 100° C. under Ar (g) for 5 hours. The reaction mixture was concentrated in vacuo and the residue was purified by silica gel chromatography (MeOH/DCM=0 to 30%) to afford methyl 2,3-difluoro-5-[(4-oxo-7-prop-1-ynyl-3H-phthalazin-1-yl)methyl]benzoate as a white solid (52 mg, 44% yield). LCMS ESI m/z: 369 [M+H]⁺.

### Step 6

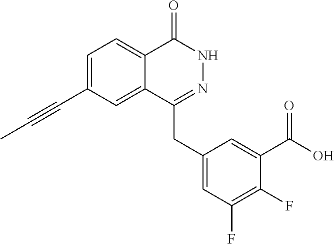 [SMILES: CC#Cc1ccc2c(=O)[nH]nc(Cc3cc(F)c(F)c(C(=O)O)c3)c2c1]

### 2,3-Difluoro-5-((4-oxo-7-(prop-1-yn-1-yl)-3,4-dihydrophthalazin-1-yl)methyl)benzoic acid

A mixture of methyl 2,3-difluoro-5-[(4-oxo-7-prop-1-ynyl-3H-phthalazin-1-yl)methyl]benzoate (52 mg, 0.14 mmol) and NaOH (17 mg, 0.42 mmol) in MeOH (2 mL). THF (2 mL) and H₂O (2 mL) was stirred at rt for 17 hours. The organic solvent was removed in vacuo and aq HCl (1N) was added until the solution reached pH 5˜6. The solids were collected by filtration, washed with water and dried under vacuum to give 2,3-difluoro-5-[(4-oxo-7-prop-1-ynyl-3H-phthalazin-1-yl)methyl]benzoic acid as a white solid (34 mg, 68% yield). LCMS ESI m/z: 355 [M+H]⁺.

### Step 7

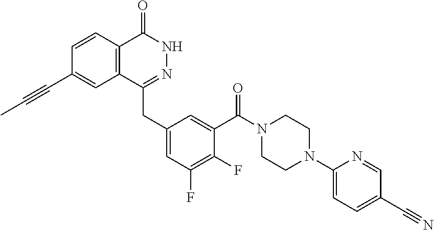 [SMILES: CC#Cc1ccc2c(=O)[nH]nc(Cc3cc(F)c(F)c(C(=O)N4CCN(c5ccc(C#N)cn5)CC4)c3)c2c1]

### 6-(4-(2,3-Difluoro-5-((4-oxo-7-(prop-1-ynyl)-3,4-dihydrophthalazin-1-yl)methyl)benzoyl)piperazin-1-yl)nicotinonitrile

To a solution of 6-piperazin-1-ylpyridine-3-carbonitrile (36 mg, 0.16 mmol) and 2,3-difluoro-5-[(4-oxo-7-prop-1-ynyl-3H-phthalazin-1-yl)methyl]benzoic acid (32 mg, 0.09 mmol) in DMF (5 mL) was added EDCI (26 mg, 0.13 mmol), HOBt (18 mg, 0.13 mmol) and DIPEA (58 mg, 0.45 mmol), The reaction mixture was stirred at rt for 2 hours. The reaction was purified by prep-HPLC to give 6-[4-[2,3-difluoro-5-[(4-oxo-7-prop-1-ynyl-3H-phthalazin-1-yl)methyl]benzoyl]piperazin-1-yl]pyridine-3-carbonitrile as a white solid (31 mg, 65% yield). LCMS ESI m/z: 525.2 [M+H]⁺. ¹H NMR (400 MHz, CDCl₃) 10.19 (s, 1H), 8.42 (d, J=1.9 Hz, 1H), 8.37 (d, J=8.2 Hz, 1H), 7.73 (dd, J=8.2, 1.3 Hz, 1H), 7.69-7.64 (m, 2H), 7.22-7.14 (m, 1H), 7.12 (d, J=4.8 Hz, 1H), 6.62 (d, J=8.9 Hz, 1H), 4.23 (s, 2H), 3.89 (s, 2H), 3.78 (s, 2H), 3.70 (t, J=5.1 Hz, 2H), 3.44 (s, 2H), 2.09 (s, 3H).

### Synthesis of Example 75: 6-(Ethyl(2-(1-oxo-6-((4-oxo-7-(prop-1-ynyl)-3,4-dihydrophthalazin-1-yl)methyl)isoindolin-2-yl)ethyl)amino)nicotinonitrile

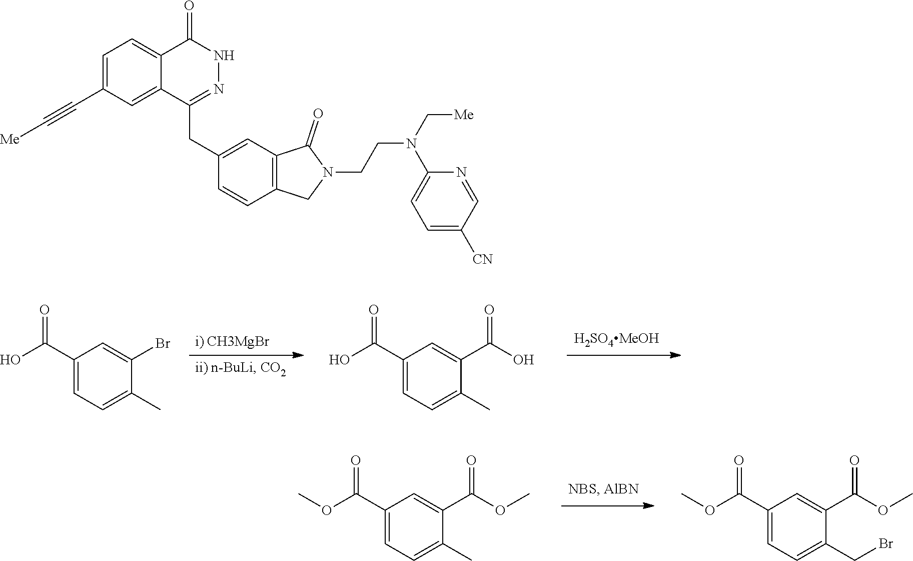

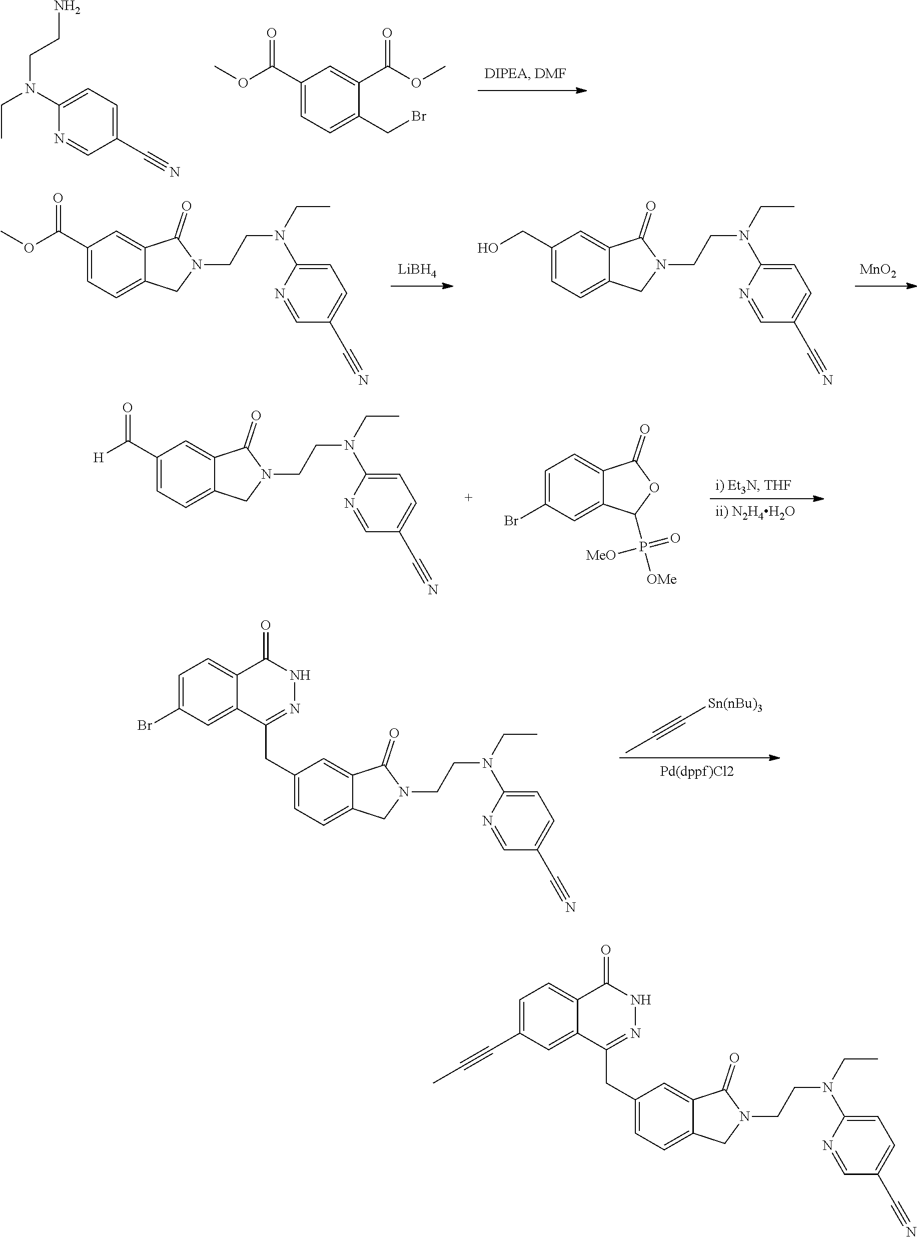

### Step 1

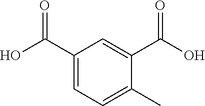

### 4-Methylisophthalic acid

To a solution of 3-bromo-4-methyl-benzoic acid (10.0 g, 46.5 mmol) in THF (50 mL) was slowly added methylmagnesium bromide (0M in THF, 51 mL, 51 mmol) at 0° C. under Ar (g). The mixture was stirred at 0° C. for 2 hours, then cooled to −78° C. and added n-BuLi (2.5 M in Hexane, 36 mL, 90 mmol) dropwise. The reaction was warmed to −40° C. under CO₂ (g) for 30 min, then stirred at rt for 12 hours. The reaction mixture was quenched with sat. aq NH₄Cl (10 mL) and extracted with EtOAc (50 mL×3). The combined organic layer was dried over Na₂SO₄, filtered and concentrated in vacuo to give 4-methylbenzene-1,3-dicarboxylic acid as a white solid (8.2 g, 43% yield). This was used without further purification. LCMS ESI m/z: 181 [M+H]⁺.

### Step 2

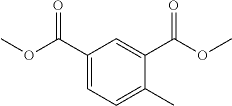

### Dimethyl 4-methylisophthalate

To a solution of 4-methylbenzene-1,3-dicarboxylic acid (7.2 g, 14.39 mmol) in MeOH (150 mL) was added conc. H₂SO₄ (5 mL) in portions. The reaction mixture was stirred at 70° C. for 8 hours, then cooled to rt, diluted with H₂O (50 mL) and extracted with EtOAc (50 mL×3). The combined organic layer was dried over Na₂SO₄, filtered and concentrated in vacuum. The residue was purified by silica gel chromatography (EtOAc/petroleum ether=10 to 20%) to afford ethyl dimethyl 4-methylisophthalate as a yellow oil (7.1 g, 85% yield). LCMS ESI m/z: 209 [M+H]⁺.

### Step 3

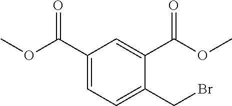

### Dimethyl 4-(bromomethyl)isophthalate

A mixture of ethyl dimethyl 4-methylisophthalate (2.6 g, 12.49 mmol), NBS (2.44 g, 13.74 mmol) and AIBN (410 mg, 2.50 mmol) in CCl₄ (20 mL) was stirred at 70° C. for 15 hours. The mixture was cooled to rt, diluted with H₂O (30 mL) and extracted with DCM (30 mL×3). The combined organic layer was dried over anhydrous Na₂SO₄, filtered and concentrated in vacuum. The residue was purified by silica gel chromatography (EtOAc/petroleum ether=0 to 10%) to obtain dimethyl 4-(bromomethyl)isophthalate as a white solid (2.11 g, 59% yield). LCMS ESI m/z: 287 (M+H)⁺.

### Step 4

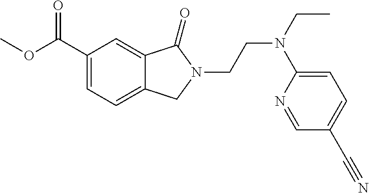 [SMILES: CCN(CCN1Cc2ccc(C(=O)OC)cc2C1=O)c1ccc(C#N)cn1]

### Methyl 2-(2-((5-cyanopyridin-2-yl)(ethyl)amino)ethyl)-3-oxoisoindoline-5-carboxylate

A solution of dimethyl 4-(bromomethyl)isophthalate (542 mg, 1.89 mmol), 6-((2-aminoethyl)(ethyl)amino)nicotinonitrile (450 mg, 1.98 mmol) and DIPEA (732 mg, 5.66 mmol) in DMF (6 mL) was stirred at rt under Ar (g) for 12 hours. The mixture was diluted with H₂O (5 mL) and extracted with EtOAc (15 mL×3). The combined organic layer was washed with brine, dried over anhydrous Na₂SO₄, filtered and concentrated in vacuo. The residue was purified by silica gel chromatography (EtOAc/petroleum ether=50 to 70%) to give methyl 2-(2-((5-cyanopyridin-2-yl)(ethyl)amino)ethyl)-3-oxoisoindoline-5-carboxylate as a colorless oil (495 mg, 79% yield). LCMS ESI m/z: 365 [M+H]⁺.

### Step 5

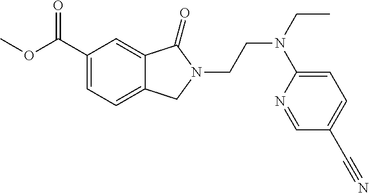 [SMILES: CCN(CCN1Cc2ccc(C(=O)OC)cc2C1=O)c1ccc(C#N)cn1]

### 6-(Ethyl(2-(6-(hydroxymethyl)-1-oxoisoindolin-2-yl)ethyl)amino)nicotinonitrile

To a mixture of methyl 2-(2-((5-cyanopyridin-2-yl)(ethyl)amino)ethyl)-3-oxoisoindoline-5-carboxylate (445 mg, 1.22 mmol) in THF (10 mL) was added LiBH₄ (2M in THF 1.22 mL, 2.44 mmol) in portions. The mixture was stirred at rt for 18 hours, then diluted with water (10 mL) and extracted with EtOAc (20 mL×3). The combined organic layer was dried over Na₂SO₄, filtered, and concentrated in vacuum. The residue was purified by silica gel chromatography (EtOAc/petroleum ether=60 to 80%) to afford 6-(ethyl(2-(6-(hydroxymethyl)-1-oxoisoindolin-2-yl)ethyl)amino)nicotinonitrile as a yellow oil (360 mg, 88% yield). LCMS ESI m/z: 337 [M+H]⁺.

### Step 6

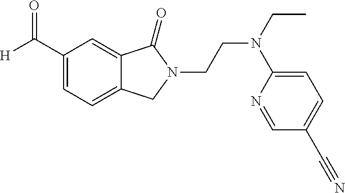 [SMILES: CCN(CCN1Cc2ccc(C=O)cc2C1=O)c1ccc(C#N)cn1]

### 6-(Ethyl(2-(6-formyl-1-oxoisoindolin-2-yl)ethyl)amino)nicotinonitrile

A mixture of 6-(ethyl(2-(6-(hydroxymethyl)-1-oxoisoindolin-2-yl)ethyl)amino)nicotinonitrile (360 mg, 1.07 mmol) and MnO₂ (464 mg, 5.34 mmol) in DCM (15 mL) was stirred at 50° C. for 12 hours. The reaction was filtered and the filtrate was concentrated in vacuo to obtain 6-(ethyl(2-(6-formyl-1-oxoisoindolin-2-yl)ethyl)amino)nicotinonitrile as a white solid (278 mg, 77% yield). This was used without further purification. LCMS ESI m/z: 335 [M+H]⁺.

### Step 7

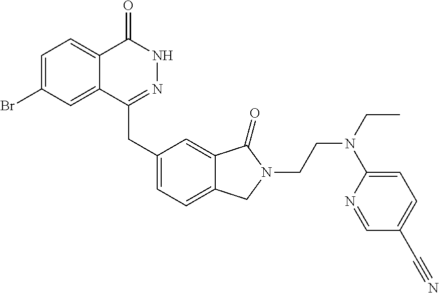 [SMILES: CCN(CCN1Cc2ccc(Cc3n[nH]c(=O)c4ccc(Br)cc34)cc2C1=O)c1ccc(C#N)cn1]

### 6-((2-(6-((7-Bromo-4-oxo-3,4-dihydrophthalazin-1-yl)methyl)-1-oxoisoindolin-2-yl)ethyl)(ethyl)amino)nicotinonitrile

A solution of 6-(ethyl(2-(6-formyl-1-oxoisoindolin-2-yl)ethyl)amino)nicotinonitrile (250 mg, 0.75 mmol), 5-bromo-3-dimethoxyphosphoryl-3H-isobenzofuran-1-one (240 mg, 0.75 mmol) and Et₃N (228 mg, 2.25 mmol) in anhydrous THF (10 mL) was stirred at 25° C. under Ar (g) for 15 hours. To the reaction mixture, hydrazine hydrate (265 mg, 4.5 mmol) was added and the reaction was stirred at 70° C. for 1 hour. The reaction was cooled to rt, then THF was removed under vacuum. The solids were washed with H₂O (10 mL) and petroleum ether/EA (2:1, 10 mL) to give 6-((2-(6-((7-bromo-4-oxo-3,4-dihydrophthalazin-1-yl)methyl)-1-oxoisoindolin-2-yl)ethyl)(ethyl)amino)nicotinonitrile as a white solid (290 mg, 84% yield). This was used without further purification. LCMS ESI m/z: 543 [M+H]⁺.

### Step 8

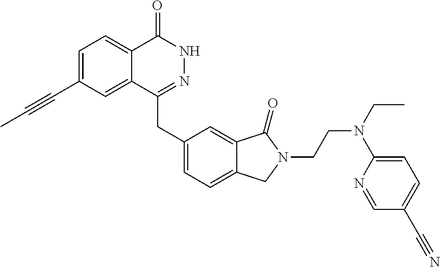 [SMILES: CC#Cc1ccc2c(=O)[nH]nc(Cc3ccc4c(c3)C(=O)N(CCN(CC)c3ccc(C#N)cn3)C4)c2c1]

### 6-(Ethyl(2-(1-oxo-6-((4-oxo-7-(prop-1-ynyl)-3,4-dihydrophthalazin-1-yl)methyl)isoindolin-2-yl)ethyl)amino)nicotinonitrile

A mixture of tributyl(prop-1-ynyl)stannane (230, mg, 0.70 mmol), 6-((2-(6-((7-bromo-4-oxo-3,4-dihydrophthalazin-1-yl)methyl)-1-oxoisoindolin-2-yl)ethyl)(ethyl)amino)nicotinonitrile (100 mg, 0.18 mmol) and Pd(dppf)Cl₂ (16 mg, 0.02 mmol) in 1,4-dioxane (5 mL) was stirred at 100° C. under Ar (g) for 5 hours. The reaction mixture was concentrated in vacuo and the residue was purified by prep-HPLC to afford methyl 2,3-difluoro-5-[(4-oxo-7-prop-1-ynyl-3H-phthalazin-1-yl)methyl]benzoate as a white solid (26 mg, 29% yield). LCMS ESI m/z: 503 [M+H]⁺. ¹H NMR (400 MHz, CDCl₃) δ 9.92 (s, 1H), 8.35 (d, J=8.8 Hz, 1H), 8.28 (d, J=2.1 Hz, 1H), 7.74-7.67 (m, 3H), 7.53 (dd, J=9.0, 2.3 Hz, 1H), 7.46 (d, J=7.8 Hz, 1H), 7.38 (d, J=7.8 Hz, 1H), 6.53 (d, J=9.1 Hz, 1H), 4.44 (s, 2H), 4.33 (s, 2H), 3.90-3.79 (m, 4H), 3.52 (q, J=7.1 Hz, 2H), 2.09 (s, 3H), 1.19 (t, J=7.1 Hz, 3H).

### Synthesis of Example 76: 2-Fluoro-5-((4-oxo-7-(prop-1-yn-1-yl)-3,4-dihydrophthalazin-1-yl)methyl)benzonitrile

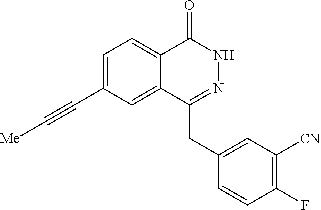 [SMILES: CC#Cc1ccc2c(=O)[nH]nc(Cc3ccc(F)c(C#N)c3)c2c1]

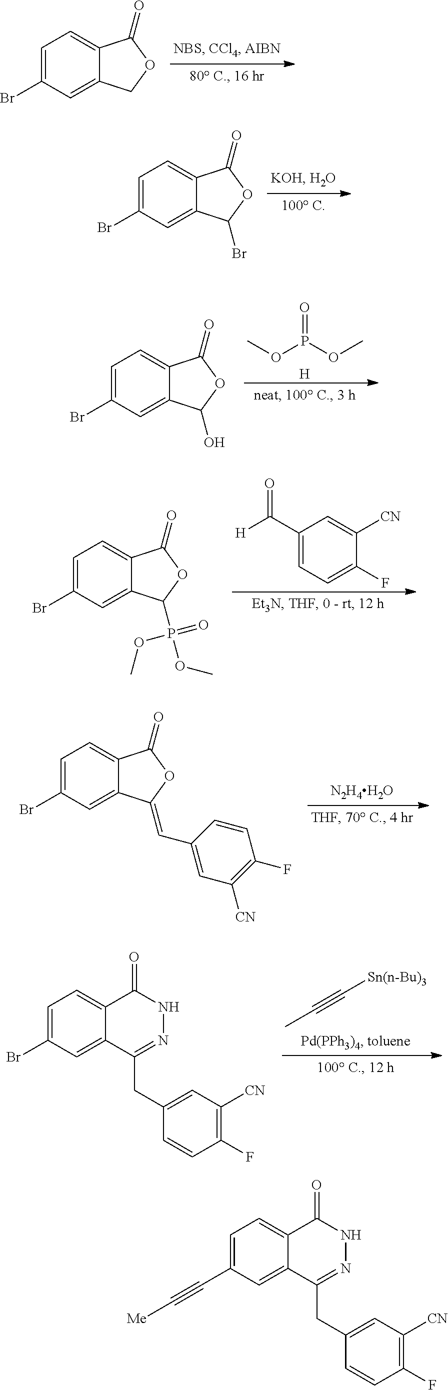

### Step 1

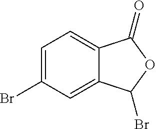 [SMILES: O=C1OC(Br)c2cc(Br)ccc21]

3,5-Dibromoisobenzofuran-1(3H)-one To a solution of 5-bromo-3H-isobenzofuran-1-one (10.0 g, 46.9 mmol) in CCl₄ (100 mL) was added AIBN (771 mg, 4.7 mmol) and 1-bromopyrrolidine-2,5-dione (10.0 g, 56.3 mmol), The resulting mixture was stirred at 80° C. for 16 hours. Water (90 mL) and EtOAc (120 mL) were added to the mixture, which became two phases. The organic layer was washed with brine, dried over Na₂SO₄ and concentrated to give a residue. The residue was purified by silica gel chromatography (petroleum ether/EtOAc=10/1) to give 3,5-dibromo-3H-isobenzofuran-1-one (9 g, 66% yield) as a white solid. ¹H-NMR (400 MHz, CDCl₃) δ 7.80-7.78 (m, 1H), 7.78 (s, 1H), 7.76 (d, J=1.3 Hz, 1H), 7.34 (s, 1H).

### Step 2

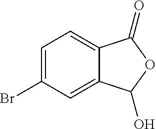 [SMILES: O=C1OC(O)c2cc(Br)ccc21]

### 5-Bromo-3-hydroxyisobenzofuran-1(3H)-one

To a mixture of 3,5-dibromo-3H-isobenzofuran-1-one (8.00 g, 27.4 mmol) in water (50 mL) was added KOH (3.08 g, 54.8 mmol), The resulting mixture was stirred at 100° C. for 2 hours. The mixture was cooled to rt, acidified with 1 M HCl until the solution reached pH 5˜6. The mixture was filtered, the filter cake was washed with H₂O (20 mL×2) and dried under reduced pressure to give 5-bromo-3-hydroxy-3H-isobenzofuran-1-one (5.4 g, 86% yield) as a white solid. ¹H-NMR (400 MHz, DMSO-d₆) δ 8.28 (d, J=8.2 Hz, 1H), 7.93 (s, 1H), 7.86 (dd, J=8.1, 1.4 Hz, 1H), 7.77 (d, J=8.1 Hz, 1H), 6.65 (d, J=8.1 Hz, 1H).

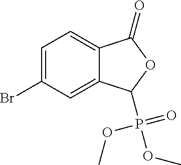 [SMILES: COP(=O)(OC)C1OC(=O)c2ccc(Br)cc21]

### Dimethyl 6-bromo-3-oxo-1,3-dihydroisobenzofuran-1-ylphosphonate

5-bromo-3-hydroxy-3H-isobenzofuran-1-one (5.00 g, 21.8 mmol) was added into dimethylphosphite (15 mL) and the resulting mixture was stirred at 100° C. for 3 hours. The mixture was quenched with H₂O (50 mL) and extracted with EtOAc (60 mL×2). The combined organic phase was washed with H₂O (40 mL×4), dried over Na₂SO₄, filtered and concentrated under reduced pressure to give the crude product, which was purified by silica gel chromatography (petroleum ether/EtOAc=30/1 to 1/1) to afford dimethyl 6-bromo-3-oxo-1,3-dihydroisobenzofuran-1-ylphosphonate (4.00 g, 57% yield) as a white solid. ¹H-NMR (400 MHz, DMSO-d₆) δ 7.89 (d, J=0.8 Hz, 2H), 7.86-7.83 (m, 1H), 6.35 (dd, J=11.6, 0.7 Hz, 1H), 3.82 (d, J=10.9 Hz, 3H), 3.65 (d, J=10.7 Hz, 3H).

### Step 4

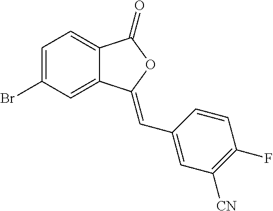 [SMILES: N#Cc1cc(/C=C2\OC(=O)c3ccc(Br)cc32)ccc1F]

### 5-[(Z)-(6-Bromo-3-oxo-isobenzofuran-1-ylidene)methyl]-2-fluoro-benzonitrile

To a mixture of 5-bromo-3-dimethoxyphosphoryl-3H-isobenzofuran-1-one (3.00 g, 9.34 mmol) in THF (40 mL) was added 2-fluoro-5-formyl-benzonitrile (2.79 g, 18.69 mmol) and N,N-diethylethanamine (2.84 g, 28.03 mmol, 3.91 mL). The resulting mixture was stirred at 25° C. for 12 hours. The mixture was quenched with sat. NaHSO₃ solution (30 mL) and filtered. The filter cake was washed with H₂O (20 mL×2) and dried under reduced pressure to give 5-[(Z)-(6-bromo-3-oxo-isobenzofuran-1-ylidene)methyl]-2-fluoro-benzonitrile (3.00 g, 93% yield) as a white solid. ¹H-NMR (400 MHz, DMSO-d₆) δ 8.42 (d, J=1.0 Hz, 1H), 8.20-8.07 (m, 2H), 7.94-7.85 (m, 2H), 7.67 (t, J=9.1 Hz, 1H), 7.08 (s, 1H).

### Step 5

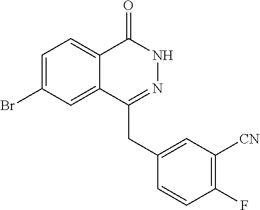 [SMILES: N#Cc1cc(Cc2n[nH]c(=O)c3ccc(Br)cc23)ccc1F]

### 5-[(7-Bromo-4-oxo-3H-phthalazin-1-yl)methyl]-2-fluoro-benzonitrile

To a mixture of 5-[(Z)-(6-bromo-3-oxo-isobenzofuran-1-ylidene)methyl]-2-fluoro-benzonitrile (3.00 g, 8.72 mmol) in THF (30 mL) was added hydrazine;hydrate (818 mg, 13.1 mmol, 797 μL). The resulting mixture was stirred at 70° C. for 4 hours. The mixture was cooled to rt, and filtered. The filter cake was washed with a solution of petrol ether/EtOAc=10/1 (20 mL×2) and dried under reduced pressure to give 5-[(7-bromo-4-oxo-3H-phthalazin-1-yl)methyl]-2-fluoro-benzonitrile (3.00 g, 77% yield) as a yellow solid. ¹H-NMR (400 MHz, DMSO-d₆) δ 8.22 (d, J=1.7 Hz, 1H), 8.17 (d, J=8.5 Hz, 1H), 8.02 (dd, J=8.5, 1.8 Hz, 1H), 7.90 (dd, J=6.2, 2.2 Hz, 1H), 7.76-7.68 (m, 1H), 7.48 (t, J=9.1 Hz, 1H), 4.38 (s, 2H).

### Step 6

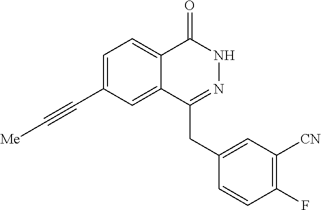 [SMILES: CC#Cc1ccc2c(=O)[nH]nc(Cc3ccc(F)c(C#N)c3)c2c1]

### 2-Fluoro-5-((4-oxo-7-(prop-1-yn-1-yl)-3,4-dihydrophthalazin-1-yl)methyl)benzonitrile

5-[(7-bromo-4-oxo-3H-phthalazin-1-yl)methyl]-2-fluoro-benzonitrile (1.50 g, 4.19 mmol), tributyl(prop-1-ynyl)stannane (2.76 g, 8.38 mmol), Pd(PPh₃)₄ (484 mg, 419 μmol) and toluene (20 mL) were added to a 100 mL bottled flask. The mixture was purged with N₂ (g) for 3 times. The resultant mixture was stirred at 100° C. for 12 hours. The mixture was filtered and concentrated under reduced pressure to give the crude product, which was purified by prep-HPLC to afford 2-fluoro-5-((4-oxo-7-(prop-1-yn-1-yl)-3,4-dihydrophthalazin-1-yl)methyl)benzonitrile (6.7 mg, 8% yield) as a white solid. LCMS ESI m/z: 317.9 [M+H]⁺. ¹H-NMR (400 MHz, DMSO-d₆) δ 12.59 (s, 1H), 8.20 (d, J=8.2 Hz, 1H), 7.97 (s, 1H), 7.88 (dd, J=6.3, 2.2 Hz, 1H), 7.79 (dd, J=8.2, 1.4 Hz, 1H), 7.75-7.69 (m, 1H), 7.47 (t, J=9.1 Hz, 1H), 4.37 (s, 2H), 2.12 (s, 3H).

### Synthesis of Example 77: 6-(4-(2,4-Difluoro-5-((4-oxo-7-(prop-1-yn-1-yl)-3,4-dihydrophthalazin-1-yl)methyl)benzoyl)piperazin-1-yl)nicotinonitrile

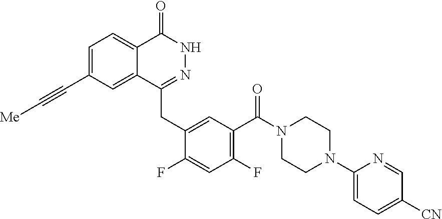 [SMILES: CC#Cc1ccc2c(=O)[nH]nc(Cc3cc(C(=O)N4CCN(c5ccc(C#N)cn5)CC4)c(F)cc3F)c2c1]

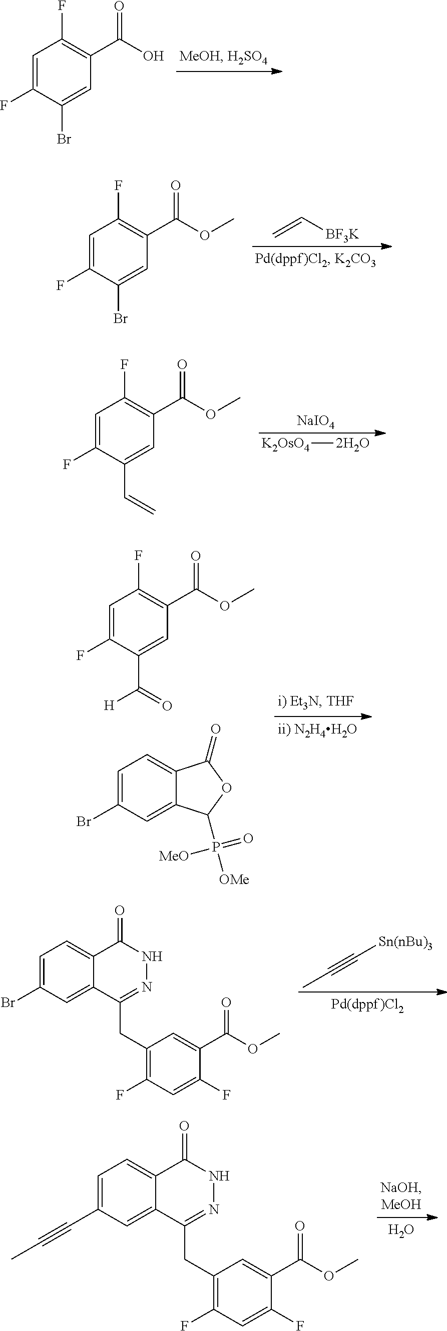

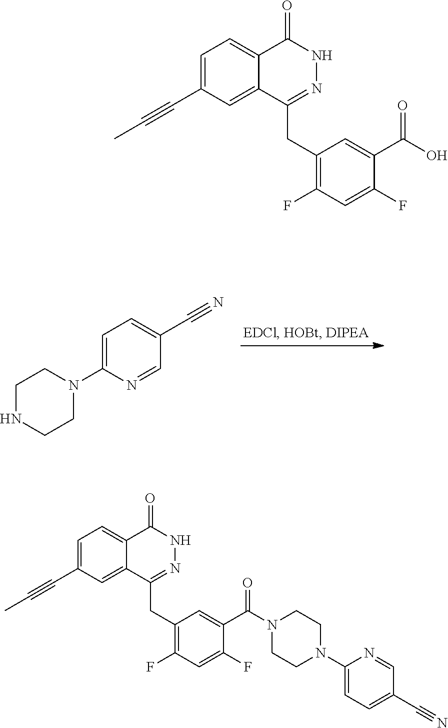 [SMILES: CC#Cc1ccc2c(=O)[nH]nc(Cc3cc(C(=O)O)c(F)cc3F)c2c1 ; N#Cc1ccc(N2CCNCC2)nc1 ; CC#Cc1ccc2c(=O)[nH]nc(Cc3cc(C(=O)N4CCN(c5ccc(C#N)cn5)CC4)c(F)cc3F)c2c1]

### Step 1

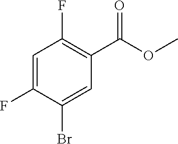

### Methyl 5-bromo-2,4-difluorobenzoate

To a solution of 5-bromo-2,4-difluoro-benzoic acid (2.00 g, 8.44 mmol) in MeOH (30 mL) was added conc. H₂SO₄ (2 mL) in portions. The mixture was heated to 70° C. for 2 hours. The solution was concentrated in vacuo. The residue was diluted with water (30 mL) and extracted with EtOAc (20 mL×3). The combined organic layer was washed with brine, dried over anhydrous Na₂SO₄, filtered and concentrated in vacuo. The residue was purified by silica gel chromatography (EtOAc/petroleum ether=0 to 4%) to afford methyl 5-bromo-2,4-difluoro-benzoate as a colorless liquid (1.87 g, 86% yield). LCMS ESI m/z: 251 [M+H]⁺.

### Step 2

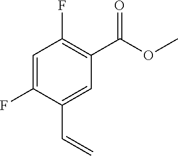

### Methyl 2,4-difluoro-5-vinylbenzoate

A mixture of methyl 5-bromo-2,4-difluoro-benzoate (1.87 g, 7.45 mmol), potassium vinyltrifluoroborate (1.99 g, 14.9 mmol), K₂CO₃ (4.12 g, 29.8 mmol) and Pd(dppf)Cl₂ (545 mg, 0.75 mmol) in MeCN (30 mL) and H₂O (8 mL) was stirred at 80° C. under Ar (g) for 2 hours. The mixture was cooled to rt, diluted with H₂O (5 mL) and extracted with EtOAc (10 mL×3). The combined organic layer was washed with brine, dried over anhydrous Na₂SO₄, filtered and concentrated in vacuo. The residue was purified by silica gel chromatography (EtOAc/petroleum ether=0 to 7%) to give methyl 2,4-difluoro-5-vinyl-benzoate as a colorless oil (1.26 g, 85% yield). LCMS ESI m/z: 199.1 [M+H]⁺. ¹H NMR (400 MHz, CDCl₃) δ 8.10 (t, J=8.0 Hz, 1H), 6.88-6.73 (m, 2H), 5.84 (d, J=16 Hz, 1H), 5.42 (d, J=12 Hz, 1H), 3.93 (s, 3H).

### Step 3

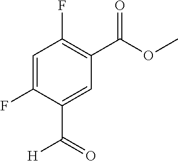

### Methyl 2,4-difluoro-5-formylbenzoate

To a mixture of methyl 2,4-difluoro-5-vinyl-benzoate (1.26 g, 6.36 mmol) in THF (20 mL) and H₂O (15 mL) was added K₂OsO₄-2H₂O (23 mg, 0.06 mmol), The mixture was stirred at room temperature for 5 min, then NaIO₄ (3.98 g, 18.62 mmol) was added and the mixture was stirred at rt for 2 hours. The solids were removed by filtration. The filtrate was diluted with H₂O (5 mL) and extracted with EtOAc (30 mL×3). The combined organic layer was washed with brine, dried over anhydrous Na₂SO₄, filtered and concentrated in vacuo to give methyl 2,4-difluoro-5-formyl-benzoate (1.14 g, 92% yield). This was used without further purification. LCMS ESI m/z: 201[M+H]⁺.

### Step 4

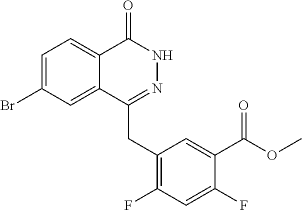 [SMILES: COC(=O)c1cc(Cc2n[nH]c(=O)c3ccc(Br)cc23)c(F)cc1F]

### Methyl 5-((7-bromo-4-oxo-3,4-dihydrophthalazin-1-yl)methyl)-2,4-difluorobenzoate

A solution of 5-bromo-3-dimethoxyphosphoryl-3H-isobenzofuran-1-one (642 mg, 2.00 mmol), methyl 2,4-difluoro-5-formyl-benzoate (400 mg, 2.00 mmol) and Et₃N (607 mg, 6.00 mmol) in anhydrous THF (16 mL) was stirred at 25° C. under Ar (g) for 3 hours. Hydrazine hydrate (220 mg, 3.73 mmol) was added and the reaction was stirred at 70° C. for 2 hour. The reaction was cooled to rt, then solvent was removed under vacuum. The solids were washed with water (10 mL) and petroleum ether/EtOAc (10:1, 10 mL) to give methyl 5-((7-bromo-4-oxo-3,4-dihydrophthalazin-1-yl)methyl)-2,4-difluorobenzoate as a yellow solid (664 mg, 82% yield). LCMS ESI m/z: 408.9 [M+H]⁺.

### Step 5

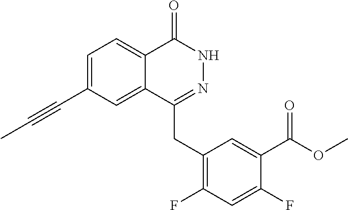 [SMILES: CC#Cc1ccc2c(=O)[nH]nc(Cc3cc(C(=O)OC)c(F)cc3F)c2c1]

### Methyl 2,4-Difluoro-5-((4-oxo-7-(prop-1-ynyl)-3,4-dihydrophthalazin-1-yl)methyl)benzoate

A mixture of tributyl(prop-1-ynyl)stannane (354 mg, 1.08 mmol), methyl 5-[(7-bromo-4-oxo-3H-phthalazin-1-yl)methyl]-2,3-difluoro-benzoate (200 mg, 0.49 mmol) and Pd(dppf)Cl₂ (25 mg, 0.04 mmol) in 1,4-dioxane (5 mL) were stirred at 100° C. under Ar (g) for 3 hours. The reaction mixture was concentrated in vacuo and the residue was purified by silica gel chromatography (MeOH/DCM 0-30%) to afford methyl 2,4-difluoro-5-((4-oxo-7-(prop-1-ynyl)-3,4-dihydrophthalazin-1-yl)methyl)benzoate as a yellow solid (97 mg, 54% yield). LCMS ESI m/z: 369 [M+H]⁺.

### Step 6

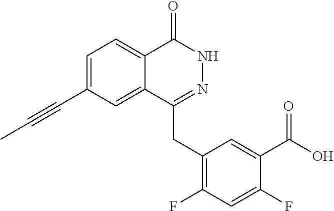 [SMILES: CC#Cc1ccc2c(=O)[nH]nc(Cc3cc(C(=O)O)c(F)cc3F)c2c1]

### 2,4-Difluoro-5-((4-oxo-7-(prop-1-yn-1-yl)-3,4-dihydrophthalazin-1-yl)methyl)benzoic acid

A mixture of methyl 2,4-difluoro-5-((4-oxo-7-(prop-1-ynyl)-3,4-dihydrophthalazin-1-yl)methyl)benzoate (97 mg, 0.26 mmol) and NaOH (32 mg, 0.79 mmol) in MeOH (2 mL), THF (2 mL) and H₂O (2 mL) was stirred at rt for 1 hour. The organic solvent was removed in vacuum and aq HCl (1N) was added until the solution reached to pH 5˜6. The solids were collected by filtration, washed with water and dried under vacuum to give 2,4-difluoro-5-((4-oxo-7-prop-1-yn-1-yl)-3,4-dihydrophthalazin-1-yl)methyl)benzoic acid as a white solid (60 mg, 67% yield). LCMS ESI m/z: 355 [M+H]⁺.

### Step 7

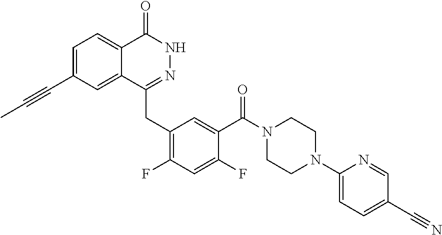 [SMILES: CC#Cc1ccc2c(=O)[nH]nc(Cc3cc(C(=O)N4CCN(c5ccc(C#N)cn5)CC4)c(F)cc3F)c2c1]

### 6-(4-(2,4-Difluoro-5-((4-oxo-7-(prop-1-ynyl)-3,4-dihydrophthalazin-1-yl)methyl)benzoyl)piperazin-1-yl)nicotinonitrile

To a solution of 6-piperazin-1-ylpyridine-3-carbonitrile (69 mg, 0.30 mmol) and 2,4-difluoro-5-((4-oxo-7-(prop-1-yn-1-yl)-3,4-dihydrophthalazin-1-yl)methyl)benzoic acid (60 mg, 0.17 mmol) in DMF (4 mL) was added EDCI (49 mg, 0.25 mmol), HOBT (34 mg, 0.25 mmol) and DIPEA (109 mg, 0.85 mmol), The reaction mixture was stirred at rt for 2 hours. The reaction was purified by prep-HPLC to give 6-(4-(2,4-difluoro-5-((4-oxo-7-(prop-1-ynyl)-3,4-dihydrophthalazin-1-yl)methyl)benzoyl)piperazin-1-yl)nicotinonitrile as a white solid (35 mg, 42% yield). LCMS ESI m/z: 525 [M+H]⁺. ¹H NMR (400 MHz, CDCl₃) δ 10.17 (s, 1H), 8.41 (d, J=2.0 Hz, 1H), 8.35 (d, J=8.2 Hz, 1H), 7.77 (s, 1H), 7.74 (d, J=8.3 Hz, 1H), 7.65 (dd, J=9.0, 2.2 Hz, 1H), 7.31 (t, J=7.7 Hz, 1H), 6.94 (t, J=9.3 Hz, 1H), 6.61 (d, J=9.0 Hz, 1H), 4.24 (s, 2H), 3.86 (s, 2H), 3.72 (d, J=28.1 Hz, 4H), 3.43 (s, 2H), 2.12 (s, 3H).

### Synthesis of Example 78: 6-(Ethyl(2-((2,2,2-trifluoro-1-(2-fluoro-5-((4-oxo-7-(prop-1-yn-1-yl)-3,4-dihydrophthalazin-1-yl)methyl)phenyl)ethyl)amino)ethyl)amino)nicotinonitrile

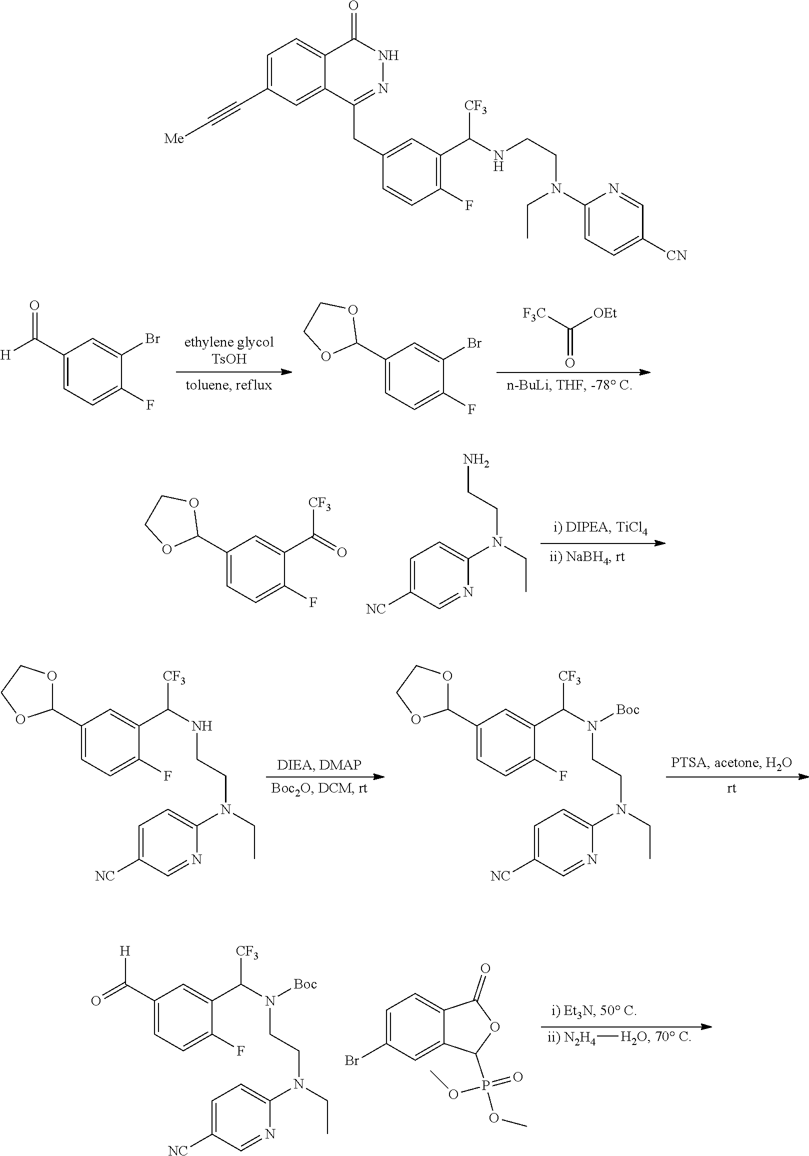

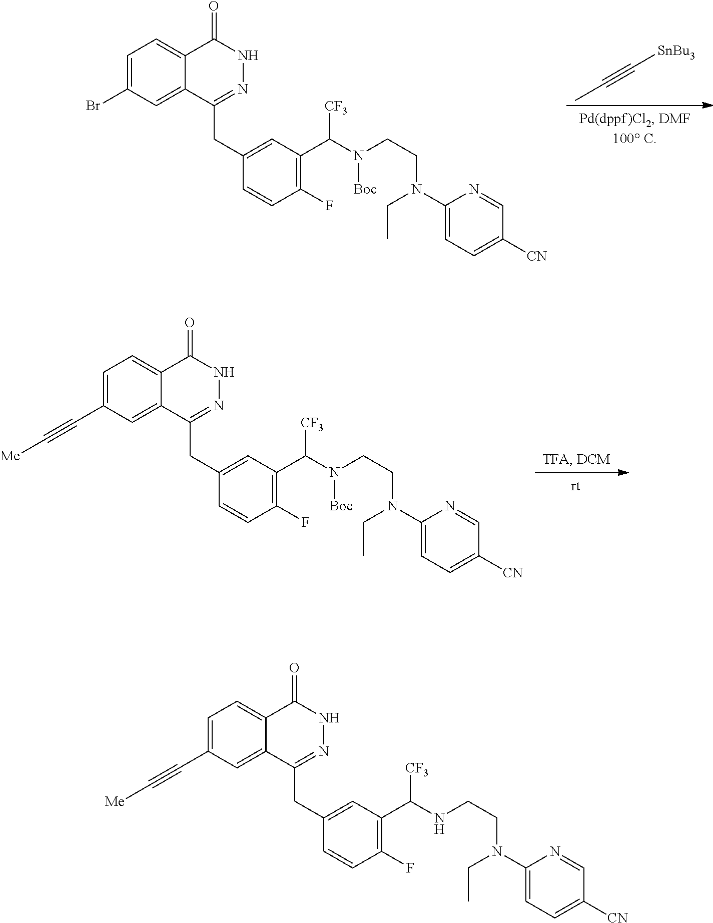

### Step 1

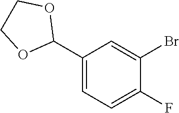 [SMILES: Fc1ccc(C2OCCO2)cc1Br]

### 2-(3-Bromo-4-fluorophenyl)-1,3-dioxolane

To a mixture of 3-bromo-4-fluorobenzaldehyde (5.00 g, 24.6 mmol), ethylene glycol (22.9 g, 369 mmol) and TsOH (2.12 g, 12.3 mmol) in toluene (60 mL) was stirred under reflux for 2 hour. The mixture was cooled to t, diluted with H₂O (10 mL) and extracted with EtOAc (20 mL×3). The combined organic layer was washed with brine, dried over anhydrous Na₂SO₄, and filtered and concentrated in vacuo. The residue was purified by prep-HPLC to give 2-(3-bromo-4-fluorophenyl)-1,3-dioxolane (5.44 g, 89/o yield) as a white solid. LCMS ESI m/z: 247/249 [M+H]⁺.

### Step 2

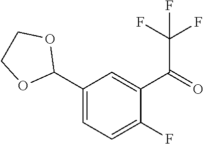 [SMILES: O=C(c1cc(C2OCCO2)ccc1F)C(F)(F)F]

### 1-(5-(1,3-Dioxolan-2-yl)-2-fluorophenyl)-2,2,2-trifluoroethan-1-one

A solution of 2-(3-bromo-4-fluorophenyl)-1,3-dioxolane (2.00 g, 8.10 mmol) in THF (10 mL) was cooled to −78° C., then n-BuLi (2.5M in heptane, 3.88 mL, 9.71 mmol) was added dropwise under Ar (g). The reaction was stirred at −78° C. for 15 min, then ethyl 2,2,2-trifluoroacetate (1.50 g, 10.52 mmol) was added dropwise. The reaction was kept at −78° C. for 20 min, then quenched with sat. NH₄Cl solution (10 mL) and neutralized with aq HCl (1 mol/L) until the solution reached pH 5˜6. The mixture was extracted with EtOAc (50×3 mL). The combined organic layer was washed with bine, dried over Na₂SO₄, filtered and concentrated in vacuo. The residue was purified by silica gel chromatography (EtOAc/petroleum ether=20% to 30%) to give 1-(5-(1,3-dioxolan-2-yl)-2-fluorophenyl)-2,2,2-trifluoroethan-1-one (950 mg, 44% yield) as a white solid. LCMS ESI m/z: 265 [M+H]⁺.

### Step 3

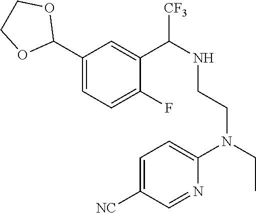 [SMILES: CCN(CCNC(c1cc(C2OCCO2)ccc1F)C(F)(F)F)c1ccc(C#N)cn1]

### 6-((2-((1-(5-(1,3-Dioxolan-2-yl)-2-fluorophenyl)-2,2,2-trifluoroethyl)amino)ethyl) (ethyl)amino)nicotinonitrile

A solution of dimethyl 1-(5-(1,3-dioxolan-2-yl)-2-fluorophenyl)-2,2,2-trifluoroethan-1-one (450 mg, 1.70 mmol), 6-((2-aminoethyl)(ethyl) amino)nicotinonitrile (463 mg, 2.04 mmol), TiCl₄ (323 mg, 1.70 mmol) and DIPEA (880 mg, 6.81 mmol) in anhydrous DCM (5 mL) was stirred at 25° C. under Ar (g) for 1 hours. NaBH₄ (322 mg, 8.52 mmol) was added and the reaction was stirred at 25° C. for 2 hour. The mixture was diluted with H₂O (10 mL) and extracted with DCM (20 mL×3). The combined organic layer was dried over anhydrous Na₂SO₄, filtered and concentrated in vacuo. The residue was purified by silica gel chromatography (EtOAc/petroleum ether=10% to 20%) to give 6-((2-((1-(5-(1,3-dioxolan-2-yl)-2-fluorophenyl)-2,2,2-trifluoroethyl)amino)ethyl) (ethyl) amino)nicotinonitrile (280 mg, 37% yield) as a yellow oil. LCMS ESI m/z: 439 [M+H]⁺.

### Step 4

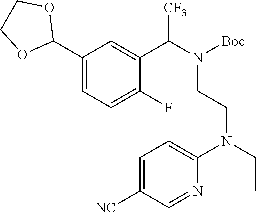 [SMILES: CCN(CCN(C(=O)OC(C)(C)C)C(c1cc(C2OCCO2)ccc1F)C(F)(F)F)c1ccc(C#N)cn1]

### tert-Butyl (1-(5-(1,3-dioxolan-2-yl)-2-fluorophenyl)-2,2,2-trifluoroethyl)(2-((5-cyanopyridin-2-yl)ethyl)amino)ethyl)carbamate

A mixture of 6-((2-((1-(5-(1,3-dioxolan-2-yl)-2-fluorophenyl)-2,2,2-trifluoroethyl)amino) ethyl)(ethyl)amino)nicotinonitrile (295 mg, 0.67 mmol), Boc₂O (146 mg, 0.67 mmol), DIPEA (87 mg, 0.67 mmol) and DMAP (82 mg, 0.67 mmol) in DCM (6 mL) was stirred at 25° C. under Ar (g) for 12 hours. The mixture was diluted with H₂O (5 mL) and extracted with DCM (10 mL×3). The combined organic layer was dried over anhydrous Na₂SO₄, filtered and concentrated in vacuo. The residue was purified by silica gel chromatography (EtOAc/petroleum ether=0% to 7%) to give tert-butyl (1-(5-(1,3-dioxolan-2-yl)-2-fluorophenyl)-2,2,2-trifluoroethyl)(2-((5-cyanopyridin-2-yl)(ethyl)amino)ethyl)carbamate (240 mg, 66% yield) as a colorless oil. LCMS ESI m/z: 539 [M+H]⁺.

### Step 5

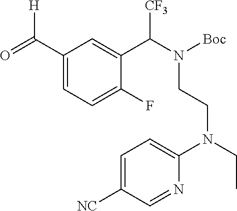 [SMILES: CCN(CCN(C(=O)OC(C)(C)C)C(c1cc(C=O)ccc1F)C(F)(F)F)c1ccc(C#N)cn1]

### tert-Butyl (2-((5-cyanopyridin-2-yl)(ethyl)amino)ethyl)(2,2,2-trifluoro-1-(2-fluoro-5-formylphenyl)ethyl)carbamate

A solution of tert-butyl (1-(5-(1,3-dioxolan-2-yl)-2-fluorophenyl)-2,2,2-trifluoroethyl)(2-((5-cyanopyridin-2-yl)(ethyl)amino)ethyl)carbamate (240 mg, 0.45 mmol) and PTSA (2 mg, 0.01 mmol) in acetone (5 mL) and H₂O (0.5 mL) was stirred at rt for 1 hour. The organic solvent was removed in vacuum and aq HCl (1N) was added until the solution reached to pH 5˜6. The solids were collected by filtration, washed with water and dried under vacuum to give tert-butyl (2-((5-cyanopyridin-2-yl)(ethyl)amino)ethyl)(2,2,2-trifluoro-1-(2-fluoro-5-formylphenyl)ethyl)carbamate (200 mg, 90% yield) as a white solid. LCMS ESI m/z: 495 [M+H]⁺. This was used without further purification.

### Step 6

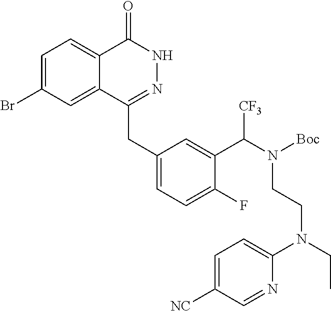 [SMILES: CCN(CCN(C(=O)OC(C)(C)C)C(c1cc(Cc2n[nH]c(=O)c3ccc(Br)cc23)ccc1F)C(F)(F)F)c1ccc(C#N)cn1]

### tert-Butyl (1-(5-((7-bromo-4-oxo-3,4-dihydrophthalazin-1-yl)methyl)-2-fluorophenyl)-2,2,2-trifluoroethyl)(2-((5-cyanopyridin-2-yl)(ethyl)amino)ethyl) carbamate

A solution of dimethyl (6-bromo-3-oxo-1,3-dihydroisobenzofuran-1-yl)phosphonate (130 mg, 0.41 mmol), tert-butyl (2-((5-cyanopyridin-2-yl)(ethyl)amino)ethyl)(2,2,2-trifluoro-1-(2-fluoro-5-formylphenyl)ethyl)carbamate (200 mg, 0.41 mmol) and Et₃N (123 mg, 1.21 mmol) in anhydrous THF (10 mL) was stirred at 50° C. under Ar (g) for 12 hours. Hydrazine hydrate (24 mg, 0.48 mmol) was added and the reaction was stirred at 70° C. for 2 hours. The reaction was cooled to rt, then THF was removed under vacuum. The formed solids were washed with water (10 mL) and petroleum ether/EtOAc (10:1, 10 mL) to give tert-butyl (1-(5-((7-bromo-4-oxo-3,4-dihydrophthalazin-1-yl)methyl)-2-fluorophenyl)-2,2,2-trifluoroethyl)(2-((5-cyanopyridin-2-yl)(ethyl)amino)ethyl)carbamate (200 mg, 89% yield) as a yellow solid. LCMS ESI m/z: 703 [M+H]⁺.

### Step 7

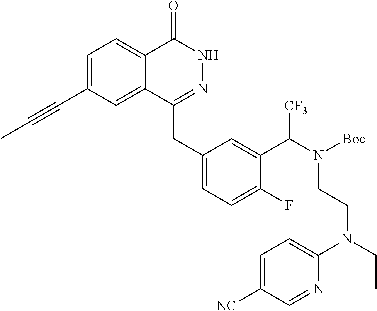 [SMILES: CC#Cc1ccc2c(=O)[nH]nc(Cc3ccc(F)c(C(N(CCN(CC)c4ccc(C#N)cn4)C(=O)OC(C)(C)C)C(F)(F)F)c3)c2c1]

### tert-Butyl (2-((5-cyanopyridin-2-yl)(ethyl)amino)ethyl)(2,2,2-trifluoro-1-(2-fluoro-5-((4-oxo-7-(prop-1-yn-1-yl)-3,4-dihydrophthalazin-1-yl)methyl)phenyl)ethyl) carbamate

A mixture of tert-butyl (1-(5-((7-bromo-4-oxo-3,4-dihydrophthalazin-1-yl)methyl)-2-fluorophenyl)-2,2,2-trifluoroethyl)(2-((5-cyanopyridin-2-yl)(ethyl)amino)ethyl)carbamate (200 mg, 0.28 mmol), tributyl(prop-1-yn-1-yl)stannane (281 mg, 0.85 mmol), Pd(dppf)Cl₂ (21 mg, 0.03 mmol) in dioxane (20 mL) was stirred at 100° C. under Ar (g) for 16 hours. The mixture was cooled to rt, diluted with H₂O (10 mL) and extracted with EtOAc (20 mL×3). The combined organic layer was washed with brine, dried over anhydrous Na₂SO₄, filtered and concentrated in vacuo. The residue was purified by silica gel chromatography (EtOAc/petroleum ether=30% to 60%) to give tert-butyl (2-((5-cyanopyridin-2-yl)(ethyl)amino)ethyl)(2,2,2-trifluoro-1-(2-fluoro-54(4-oxo-7-(prop-1-yn-1-yl)-3,4-dihydrophthalazin-1-yl)methyl)phenyl)ethyl)carbamate (160 mg, 84% yield) as a white solid. LCMS ESI m/z: 663 [M+H]⁺.

### Step 8

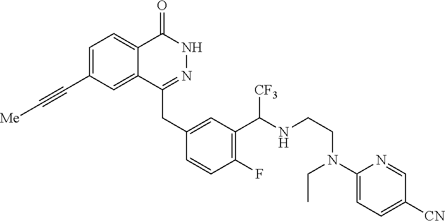 [SMILES: CC#Cc1ccc2c(=O)[nH]nc(Cc3ccc(F)c(C(NCCN(CC)c4ccc(C#N)cn4)C(F)(F)F)c3)c2c1]

### 6-(Ethyl(2-((2,2,2-trifluoro-1-(2-fluoro-5-((4-oxo-7-(prop-1-yn-1-yl)-3,4-dihydrophthalazin-1-yl)methyl)phenyl)ethyl)amino)ethyl)amino)nicotinonitrile

To a solution of tert-butyl (2-((5-cyanopyridin-2-yl)(ethyl)amino)ethyl)(2,2,2-trifluoro-1-(2-fluoro-54(4-oxo-7-(prop-1-yn-1-yl)-3,4-dihydrophthalazin-1-yl)methyl)phenyl)ethyl)carbamate (160 mg, 0.24 mmol) in DCM (5 mL) was added TFA (5 mL). The reaction mixture was stirred at rt for 1 hour. The reaction was concentrated in vacuo and the residue was purified by prep-HPLC to give the 6-(ethyl(2-((2,2,2-trifluoro-1-(2-fluoro-5-((4-oxo-7-(prop-1-yn-1-yl)-3,4-dihydrophthalazin-1-yl)methyl)phenyl)ethyl)amino)ethyl)amino)nicotinonitrile (90 mg, 66% yield) as a white solid. LCMS ESI m/z: 563 [M+H]⁺. ¹H NMR (400 MHz, DMSO-d6) δ 12.63 (s, 1H), 8.36 (s, 1H), 8.17 (d, J=8.4 Hz, 1H), 7.81 (s, 1H), 7.72-7.69 (m, 2H), 7.58-7.56 (m, 1H), 7.35-7.32 (m, 1H), 7.19 (t, J=8.8 Hz, 1H), 6.60 (d, J=9.2 Hz, 1H), 4.72-4.70 (m, 1H), 4.30-4.28 (m, 2H), 3.46-3.41 (m, 5H), 2.61 (t, J=6.8 Hz, 2H), 2.06 (s, 3H), 1.00 (t, J=6.8 Hz, 3H).

### Synthesis of Example 79: 4-(3-(4-(1,5-Dimethyl-1H-imidazol-2-yl)piperazine-1-carbonyl)-4-fluorobenzyl)-6-(prop-1-ynyl)phthalazin-1(2H)-one

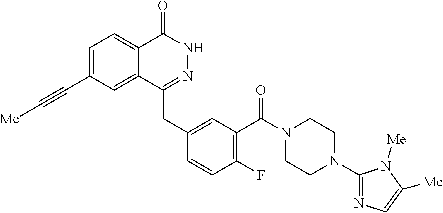 [SMILES: CC#Cc1ccc2c(=O)[nH]nc(Cc3ccc(F)c(C(=O)N4CCN(c5ncc(C)n5C)CC4)c3)c2c1]

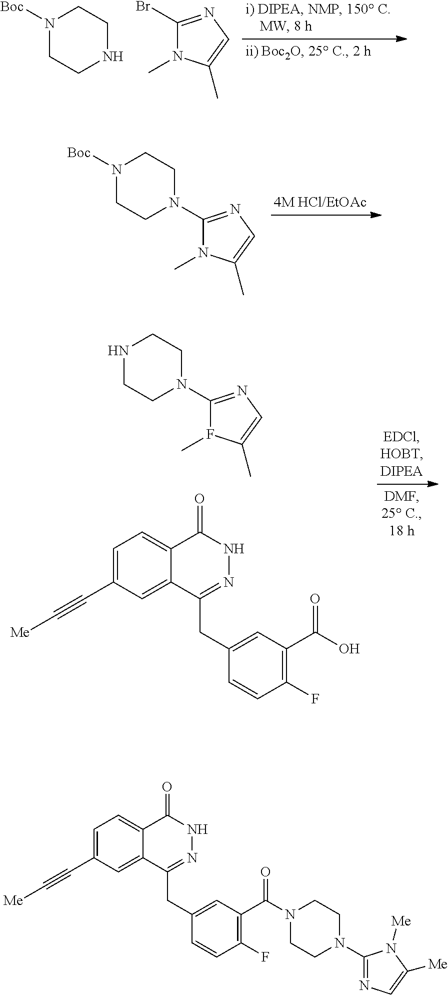

### Step 1

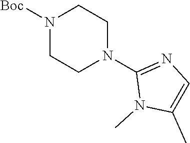 [SMILES: Cc1cnc(N2CCN(C(=O)OC(C)(C)C)CC2)n1C]

### tert-Buty 4-(1,5-dimethyl-1H-imidazol-2-yl)piperazine-1-carboxylate

To a solution of 2-bromo-1,5-dimethyl-imidazole (100 mg, 571.34 μmol) in NMP (0.5 mL) was added tert-butyl piperazine-1-carboxylate (160 mg, 857 μmol), DIPEA (369 mg, 2.86 mmol, 498 μL). The reaction mixture stirred at 150° C. under microwave for 10 hr. Di-tert-butyl dicarbonate (312 mg, 1.43 mmol, 328 μL) was added to the reaction mixture and stirred at 25° C. for 2 hr. The reaction mixture was diluted in ethyl acetate (10 mL), washed with water (10 mL), brine, dried and concentrated to the residue. The residue was purified by prepare TLC (PE/EA=0/1) to give tert-butyl 4-(1,5-dimethylimidazol-2-yl)piperazine-1-carboxylate (54 mg, 34% yield) as a white solid. ¹H NMR (400 MHz, CDCl₃) δ 6.54 (d, J=0.7 Hz, 1H), 3.55 (dd, J=6.2, 3.9 Hz, 4H), 3.37 (s, 3H), 3.07-2.94 (m, 4H), 2.14 (d, J=1.0 Hz, 3H), 1.47 (s, 9H).

### Step 2

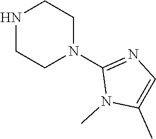

### 1-(1,5-Dimethyl-1H-imidazol-2-yl)piperazine

To a solution of tert-butyl 4-(1,5-dimethylimidazol-2-yl)piperazine-1-carboxylate (54 mg, 193 μmol) in 4M EA/HCl (3 mL). The reaction mixture was stirred at rt, for 2 hours. The mixture was concentrated to give 1-(1,5-dimethylimidazol-2-yl)piperazine (40 mg, 96% yield. HCl) as a white solid. LCMS ESI m/z: 181.1 [M+H]⁺. The crude material was used as is in the next step.

### Step 3

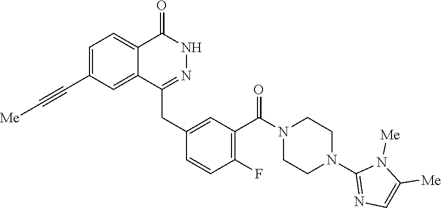 [SMILES: CC#Cc1ccc2c(=O)[nH]nc(Cc3ccc(F)c(C(=O)N4CCN(c5ncc(C)n5C)CC4)c3)c2c1]

### 4-(3-(4-(1,5-Dimethyl-1H-imidazol-2-yl)piperazine-1-carbonyl)-4-fluorobenzyl)-6-(prop-1-ynyl)phthalazin-1(2H)-one

Following the general amide coupling procedure above in Example 55, but starting with 1-(1,5-dimethyl-1H-imidazol-2-yl)piperazine gave the title compound as a white solid. LCMS ESI m/z: 498.8 [M+H]⁺. ¹H NMR (400 MHz, CDCl₃) δ 10.36 (s, 1H), 8.35 (d, J=8.2 Hz, 1H), 7.75 (s, 1H), 7.70 (d, J=8.2 Hz, 1H), 7.38 (d, J=6.3 Hz, 1H), 7.32 (s, 1H), 7.07 (t, J=8.8 Hz, 1H), 6.88 (s, 1H), 4.25 (s, 2H), 3.71-3.55 (m, 4H), 3.53 (s, 3H), 3.49-3.30 (m, 4H), 2.25 (s, 3H), 2.11 (s, 3H).

### Synthesis of Example 80: 6-(6,6-Difluoro-4-(2-fluoro-5-((4-oxo-7-(prop-1-ynyl)-3,4-dihydrophthalazin-1-yl)methyl)benzoyl)-1,4-diazepan-1-yl)nicotinonitrile

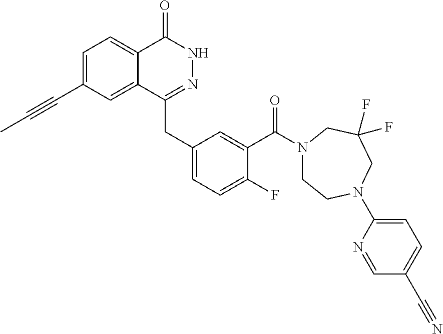 [SMILES: CC#Cc1ccc2c(=O)[nH]nc(Cc3ccc(F)c(C(=O)N4CCN(c5ccc(C#N)cn5)CC(F)(F)C4)c3)c2c1]

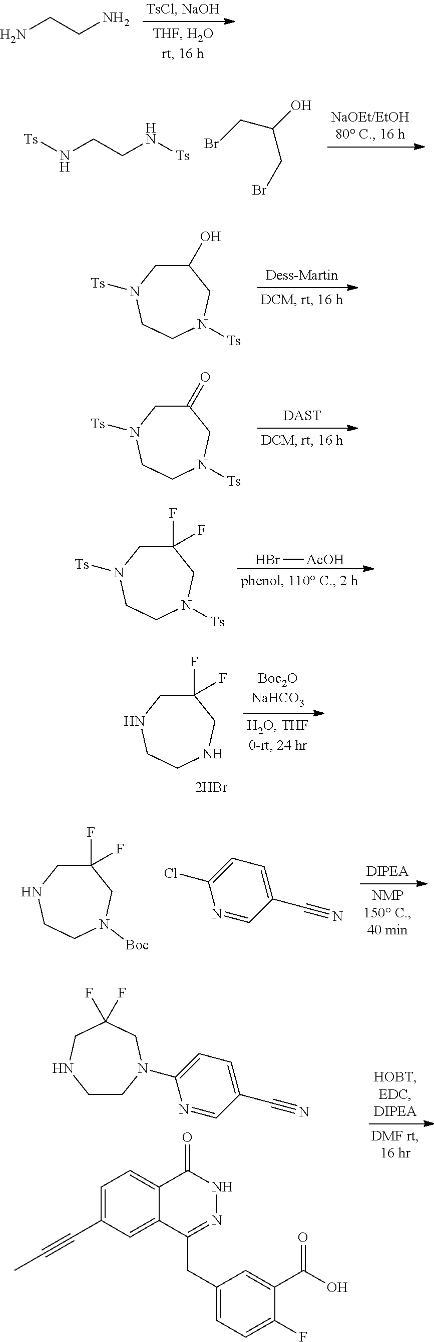

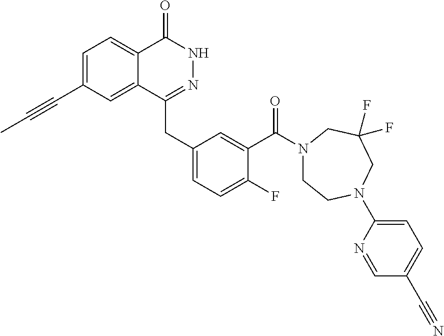 [SMILES: CC#Cc1ccc2c(=O)[nH]nc(Cc3ccc(F)c(C(=O)N4CCN(c5ccc(C#N)cn5)CC(F)(F)C4)c3)c2c1]

### Step 1

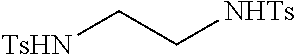

### N,N′-(Ethane-1,2-diyl)bis(4-methylbenzenesulfonamide)

To a solution of ethane-1,2-diamine (10.0 g, 166 vmmol, 11.1 mL) taken under argon atmosphere and cooled to 0° C. then NaOH (13.3 g, 333 mmol, 6.25 mL) in H₂O (134 mL) was added slowly at 0° C. The resulting mixture was dropwise added to the cooled solution of 4-methylbenzenesulfonyl chloride (63.45 g, 332.79 mmol) in THF (134 mL) at 0° C. and then stirred at 25° C. for 16 hrs. The solid suspension was filtered off and the resulting solid was filtered through sintered funnel and re-crystallized from MeOH to afford 4-methyl-N-[2-(p-tolylsulfonylamino)ethyl]benzenesulfonamide (31 g, 51%/yield) as a white solid. ¹H NMR (400 MHz, DMSO-d6) δ 7.61 (d, J=8.2 Hz, 4H), 7.38 (d, J=8.0 Hz, 4H), 2.72 (s, 4H), 2.39 (s, 6H).

### Step 2

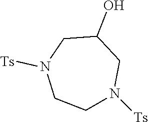 [SMILES: Cc1ccc(S(=O)(=O)N2CCN(S(=O)(=O)c3ccc(C)cc3)CC(O)C2)cc1]

### 1,4-Ditosyl-1,4-diazepan-6-ol

To a stirred solution of 4-methyl-N-[2-(p-tolylsulfonylamino)ethyl]benzenesulfonamide (14.0 g, 38.0 mmol) in ethanol (140 mL), sodium ethanolate (4.15 g, 61.0 mmol) followed by 1,3-dibromopropan-2-ol (8.53 g, 39.1 mmol) were added. Then the reaction mixture was heated to reflux at 80° C. for 16 hours. The solvent was evaporated, diluted with H₂O and neutralize the reaction mixture with citric acid and extracted with EtOAc. Combined organic layers were dried over Na₂SO₄ and concentrated. The crude obtained was again suspended in EOH-H₂O (2:1) and basified with 2 N NaOH at 0° C. carefully (pH 10). The white suspension was decanted first and then filtered, the solid residue was washed with H₂O followed by EtOH and finally dried to get 1,4-bis(p-tolylsulfonyl)-1,4-diazepan-6-ol (13.0 g, 81% yield) as a white solid. ¹H NMR (400 MHz, DMSO-d₆) δ 7.71 (d, J=8.2 Hz, 4H), 7.47 (d, J=8.1 Hz, 4H), 5.32 (d, J=4.7 Hz, 1H), 3.85-3.73 (m, 1H), 3.52 (d, J=1.8 Hz, 4H), 3.20-3.11 (m, 2H), 2.91 (dd, J=13.9, 8.1 Hz, 2H), 2.45 (s, 6H).

### Step 3

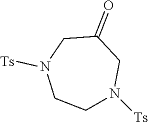 [SMILES: Cc1ccc(S(=O)(=O)N2CCN(S(=O)(=O)c3ccc(C)cc3)CC(=O)C2)cc1]

### 1,4-Ditosyl-1,4-diazepan-6-one

To a solution of 1,4-bis(p-tolylsulfonyl)-1,4-diazepan-6-ol (13 g, 30.62 mmol) in DCM (300 mL) was added DMP (26.0 g, 61.2 mmol) portion wise at 0° C. during 30 min. The reaction mixture was slowly allowed to 25° C. and stirring was continued for 15 hrs. The solids were filtered and washed with DCM. The organic layer was concentrated and purified by silica gel chromatography to afford 1,4-bis(p-tolylsulfonyl)-1,4-diazepan-6-one (12.0 g, 74% yield) as a white solid. ¹H NMR (400 MHz, DMSO-d₆) δ 7.70 (d, J=8.3 Hz, 4H), 7.43 (d, J=8.0 Hz, 4H), 3.93 (s, 4H), 3.58 (s, 4H), 2.40 (s, 6f).

### Step 4

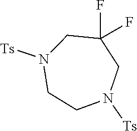 [SMILES: Cc1ccc(S(=O)(=O)N2CCN(S(=O)(=O)c3ccc(C)cc3)CC(F)(F)C2)cc1]

### 6,6-Difluoro-1,4-ditosyl-1,4-diazepane

In a polystyrene reaction vessel containing a solution of 1,4-bis(p-tolylsulfonyl)-1,4-diazepan-6-one (12.0 g, 28.4 mmol) in DCM (100 mL) was added N-ethyl-N-(trifluoro-sulfanyl)ethanamine (22.9 g, 142 mmol, 18.8 mL) at 0° C. under N₂ atmosphere. The reaction mixture was slowly allowed to warm to 25° C. and stirred for 16 h. The reaction mixture was diluted with DCM (40 ml) and carefully basified with NaHCO₃ adjust pH 7. The organic layer was separated, and aqueous layer was extracted with DCM (30 ml×2). The combined organic layers were washed with brine (30 ml), dried over Na₂SO₄ and concentrated to get crude product. The crude was purified by silica gel chromatography to afford 6,6-difluoro-1,4-bis(p-tolylsulfonyl)-1,4-diazepane (8.0 g, 63% yield) as a white solid. ¹H NMR (400 MHz, CDCl₃) δ 7.65 (d, J=8.3 Hz, 4H), 7.33 (d, J=8.0 Hz, 4H), 3.68 (t, J 12.3 Hz, 4H), 3.41 (s, 4H), 2.44 (s, 6H).

### Step 5

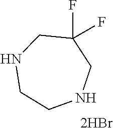

### 6,6-Difluoro-1,4-diazepane

In a polystyrA suspension of 6,6-difluoro-1,4-bis(p-tolylsulfonyl)-1,4-diazepane (1.2 g, 2.70 mmol) and phenol (1.02 g, 10.8 mmol, 949 μL) in HBr (728 mg, 2.70 mmol, 10 mL, 30% purity) was heated to 110° C. in a sealed tube. After 2 hrs, the mixture was cooled to 25° C. the solvent removed in vacuum and the residue was triturated with MTBE and washed with EtOH and finally dried to give 6,6-difluoro-1,4-diazepane (550 mg, 68% yield) as an off-white solid. ¹H NMR (400 MHz, MeOD) δ 4.07 (t, J=12.3 Hz, 4H), 3.78 (s, 4H).

### Step 6

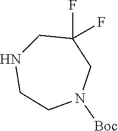 [SMILES: CC(C)(C)OC(=O)N1CCNCC(F)(F)C1]

### tert-Butyl 6,6-difluoro-1,4-diazepane-1-carboxylate

To a solution of 6,6-difluoro-1,4-diazepane (800 mg, 2.68 mmol, 2HBr) in H₂O (24 mL) was added NaHCO₃ (677 mg, 8.05 mmol), which was dissolved in THF (45 mL) at 0° C., and then Boc₂O (2.82 mmol) was added dropwise, and then stirred at rt, for 24 hrs. The mixture was concentrated and extracted with EtOAc (30 mL×2), washed with brine, dried, concentrated and purified by silica gel chromatography to give tert-butyl 6,6-difluoro-1,4-diazepane-1-carboxylate (290 mg, 46% yield) as a white solid. ¹H NMR (400 MHz, MeOD) δ 3.84 (t, J=12.5 Hz, 2H), 3.48 (s, 2H), 3.05 (t, J=13.4 Hz, 2H), 2.94-2.85 (m, 2H), 1.46 (s, 9H).

### Step 7

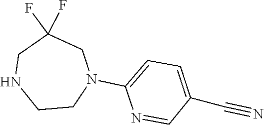 [SMILES: N#Cc1ccc(N2CCCNCC(F)(F)C2)nc1]

### 6-(6,6-Difluoro-1,4-diazepan-1-yl)nicotinonitrile

A solution of tert-butyl 6,6-difluoro-1,4-diazepane-1-carboxylate (100 mg, 423 μmol), 6-chloropyridine-3-carbonitrile (58.7 mg, 423 μmol) and DIPEA (164 mg, 1.27 mmol) in NMP (1.5 mL) was stirred at 150° C. under microwave for 40 min. The reaction mixture was diluted in EA, washed with water 3 times. The organic layer was concentrated to the crude and purified by Pre-TLC (EtOAc: Petroleum ether=3:1) to give 6-(6,6-difluoro-1,4-diazepan-1-yl)pyridine-3-carbonitrile (40 mg, 40% yield) as a yellow solid. LCMS ESI m/z: 239.2 [M+H]⁺.

### Step 8

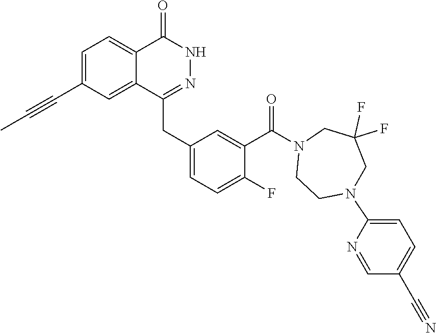 [SMILES: CC#Cc1ccc2c(=O)[nH]nc(Cc3ccc(F)c(C(=O)N4CCN(c5ccc(C#N)cn5)CC(F)(F)C4)c3)c2c1]

### 6-(6,6-Difluoro-4-(2-fluoro-5-((4-oxo-7-(prop-1-ynyl)-3,4-dihydrophthalazin-1-yl)methyl)benzoyl)-1,4-diazepan-1-yl)nicotinonitrile

Following the general amide coupling procedure in Example 55, but starting with 6-(6,6-difluoro-1,4-diazepan-1-yl)nicotinonitrile gave the title compound as a white solid. LCMS ESI m/z: 557.2 [M+H]⁺. ¹H NMR (400 MHz, DMSO-d₆) δ 12.61 (d, J=5.6 Hz, 1H), 8.51 (dd, J=35.7, 2.1 Hz, 1H), 8.20 (dd, J=8.2, 3.8 Hz, 1H), 7.92 (dt, J=21.0, 12.1 Hz, 2H), 7.77 (dd, J=11.3, 4.2 Hz, 1H), 7.43-7.34 (m, 1H), 7.30-7.18 (m, 2H), 7.00 (d, J=9.1 Hz, 1H), 4.33 (m, 4H), 4.15 (s, 1H), 3.93 (m, 3H), 3.76 (s, 1H), 3.50 (t, J=5.3 Hz, 1H), 2.09 (d, J=4.0 Hz, 3H).

### Example 81 (SP-PAR700076-NX-1)

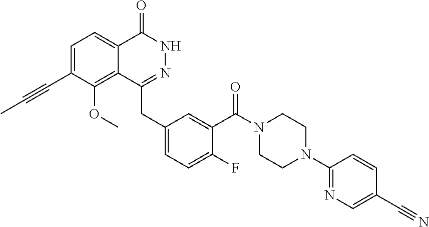 [SMILES: CC#Cc1ccc2c(=O)[nH]nc(Cc3ccc(F)c(C(=O)N4CCN(c5ccc(C#N)cn5)CC4)c3)c2c1OC]

### 6-(4-(2-Fluoro-5-((8-methoxy-4-oxo-7-(prop-1-yn-1-yl)-3,4-dihydrophthalazin-1-yl)methyl)benzoyl)piperazin-1-yl)nicotinonitrile

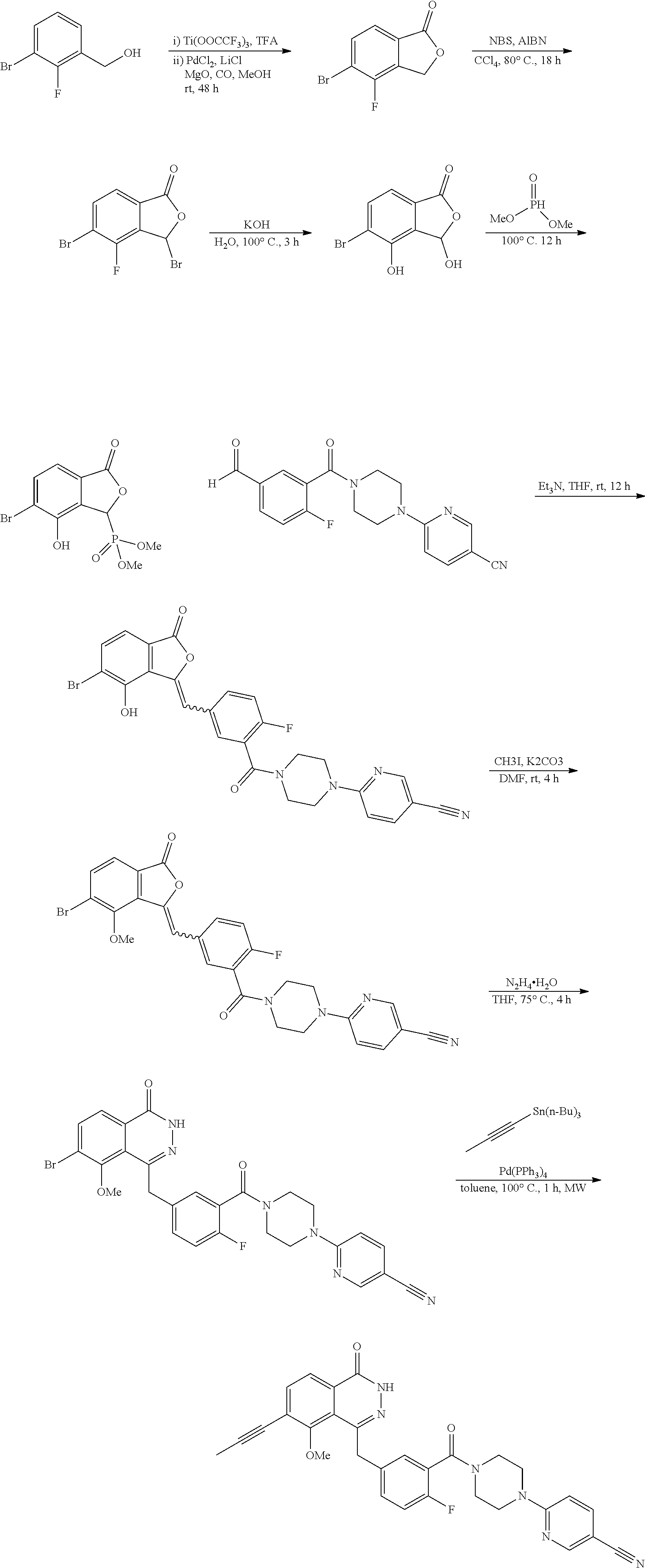

### Step 1

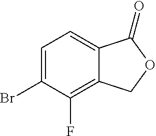 [SMILES: O=C1OCc2c1ccc(Br)c2F]

### 5-Bromo-4-fluoroisobenzofuran-1(3H)-one

A mixture of (3-bromo-2-fluorophenyl)methanol (4 g, 19.51 mmol), thallium(III) trifluoroacetate (10.71 g, 19.71 mmol) and TFA (20 mL) was stirred at room temperature for 1 day. The solution was concentrated to dryness under reduced pressure at room temperature and azeotroped with dichloromethane. The reaction mixture was then treated with PdCl₂ (345.96 mg, 1.95 mmol), LiCl (1.65 g, 39.02 mmol, 799.83 μL), MgO (1.61 g, 39.02 mmol) and MeOH (20 mL). The reaction mixture was then degassed and purged with CO several times and then stirred under CO for 1 day. EtOAc (80 mL) was added the reaction mixture to precipitate the salts. The black solution was filtered through a celite pad, washed with EtOAc (40 mL), adsorbed onto silica and purified by silica gel chromatography to afford 5-bromo-4-fluoro-3H-isobenzofuran-1-one (1 g, 22% yield) as a white solid. ¹H NMR (400 MHz, DMSO-d₆) δ 7.95 (dd, J=8.0, 5.9 Hz, 1H), 7.66 (d, J=8.1 Hz, 1H), 5.55 (s, 2H).

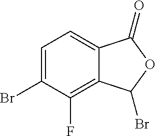 [SMILES: O=C1OC(Br)c2c1ccc(Br)c2F]

### 3,5-Dibromo-4-fluoroisobenzofuran-1(3H)-one

To a solution of 5-bromo-4-fluoro-3H-isobenzofuran-1-one (1 g, 4.33 mmol) in CCl₄ (15 mL) was added 1-bromopyrrolidine-2,5-dione (924.52 mg, 5.19 mmol, 440.67 μL), AIBN (71.08 mg, 432.87 μmol). The reaction mixture was stirred at 80° C. for 18 hours under N₂ (g). Water (20 mL) and EtOAc (30 mL) were added to the mixture. The organic layer was separated, washed with brine, dried over Na₂SO₄ and concentrated to give a residue. The residue was purified by silica gel chromatography (petroleum ether/EtOAc=20/1 to 0/1) to give 3,5-dibromo-4-fluoroisobenzofuran-1(3H)-one (1.2 g, 90% yield) as a white solid. ¹H NMR (400 MHz, DMSO-d₆) δ 7.99 (dd, J=8.0, 5.9 Hz, 1H), 7.61 (d, J=8.0 Hz, 1H), 6.82 (s, 1H).

### Step 3

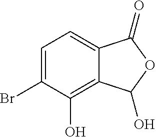 [SMILES: O=C1OC(O)c2c1ccc(Br)c2O]

### 5-Bromo-3,4-dihydroxyisobenzofuran-1(3H)-one

A solution of 3,5-dibromo-4-fluoroisobenzofuran-1(3H)-one (1.2 g, 3.87 mmol) in 1 mol/L KOH (10 mL) was stirred at 100° C. for 3 hours. The mixture was acidified with 1N HCl until the solution reached pH 2-3, extracted with EtOAc (20 mL×3). The organic layer was washed with water (20 mL), dried over Na₂SO₄, and concentrated to give 5-bromo-3,4-dihydroxyisobenzofuran-1(3H)-one (800 mg, 84% yield). The crude was taken to the next step without purification.

### Step 4

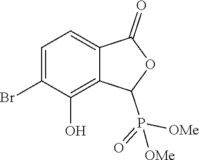 [SMILES: COP(=O)(OC)C1OC(=O)c2ccc(Br)c(O)c21]

### Dimethyl (6-bromo-7-hydroxy-3-oxo-1,3-dihydroisobenzofuran-1-yl)phosphonate

A solution of 5-bromo-3,4-dihydroxy-3H-isobenzofuran-1-one (800 mg, 3.26 mmol) in dimethyl phosphonate (1 mL) was stirred at 100° C. for 3 hours. Water (20 mL) and EtOAc (20 mL) were added to the mixture. The organic layer was separated, washed with brine, dried over Na₂SO₄ and concentrated to give a residue. The residue was purified by silica gel chromatography (petroleum ether/EtOAc=20/1 to 0/1) to give dimethyl (6-bromo-7-hydroxy-3-oxo-1,3-dihydroisobenzofuran-1-yl)phosphonate (800 mg, 73% yield) as a white solid. LCMS ESI m/z 339.0 [M+H]⁺.

### Step 5

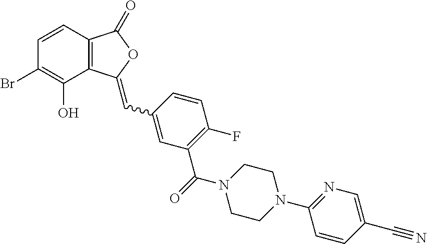 [SMILES: N#Cc1ccc(N2CCN(C(=O)c3cc(C=C4OC(=O)c5ccc(Br)c(O)c54)ccc3F)CC2)nc1]

### 6-(4-(5-((6-Bromo-7-hydroxy-3-oxoisobenzofuran-1(3H)-ylidene)methyl)-2-fluorobenzoyl)piperazin-1-yl)nicotinonitrile

To a solution of dimethyl (6-bromo-7-hydroxy-3-oxo-1,3-dihydroisobenzofuran-1-yl)phosphonate (750 mg, 2.23 mmol) in THF (7 mL) was added 6-[4-(2-fluoro-5-formyl-benzoyl)piperazin-1-yl]pyridine-3-carbonitrile (790.48 mg, 2.34 mmol), Et₃N (675.48 mg, 6.68 mmol, 930.41 μL) at 25° C. The reaction mixture was stirred at 25° C. for 12 hours. The mixture was quenched with sat. Na₂SO₃ solution (5 mL) and filtered. The filter cake was washed with H₂O (5 mL*2), dried under reduced pressure to give 6-(4-(5-((6-bromo-7-hydroxy-3-oxoisobenzofuran-1(3H)-ylidene)methyl)-2-fluorobenzoyl)piperazin-1-yl)nicotinonitrile (750 mg, 61% yield) as a yellow solid. LCMS ESI m/z 549.0 [M+H]⁺.

### Step 6

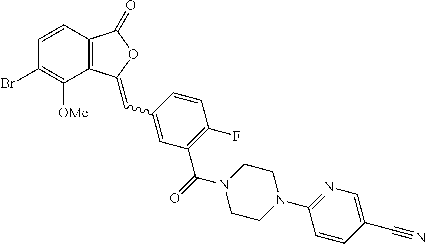 [SMILES: COc1c(Br)ccc2c1C(=Cc1ccc(F)c(C(=O)N3CCN(c4ccc(C#N)cn4)CC3)c1)OC2=O]

### 6-(4-(5-((6-Bromo-7-methoxy-3-oxoisobenzofuran-1(3H)-ylidene)methyl)-2-fluorobenzoyl)piperazin-1-yl)nicotinonitrile

To a solution of 6-(4-(5-((6-bromo-7-hydroxy-3-oxoisobenzofuran-1(3H)-ylidene)methyl)-2-fluorobenzoyl)piperazin-1-yl)nicotinonitrile (80 mg, 145.63 μmol) in DMF (1 mL) was added K₂CO₃ (60.38 mg, 436.88 μmol) and CH₃I (31.01 mg, 218.44 μmol) at 25° C. The reaction mixture was stirred at 25° C. for 4 hours. H₂O (10 mL) was added to the mixture and filtered. The filter cake was washed with water (2 mL) and dried under reduced pressure to give 6-(4-(5-((6-bromo-7-methoxy-3-oxoisobenzofuran-1(3H)-ylidene)methyl)-2-fluorobenzoyl)piperazin-1-yl)nicotinonitrile (58 mg, 71% yield).

### Step 7

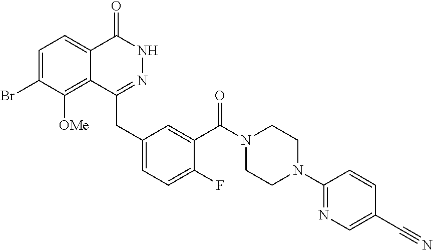 [SMILES: COc1c(Br)ccc2c(=O)[nH]nc(Cc3ccc(F)c(C(=O)N4CCN(c5ccc(C#N)cn5)CC4)c3)c12]

### 6-(4-(5-((7-Bromo-8-methoxy-4-oxo-3,4-dihydrophthalazin-1-yl)methyl)-2-fluorobenzoyl)piperazin-1-yl)nicotinonitrile

To a solution of 6-(4-(5-((6-bromo-7-methoxy-3-oxoisobenzofuran-1(3H)-ylidene)methyl)-2-fluorobenzoyl)piperazin-1-yl)nicotinonitrile (58 mg, 102.95 μmol) in THF (2 mL) was added hydrazine hydrate (12.88 mg, 205.90 μmol, 12.55 μL, 80% purity) at 25° C. under N₂ (g). The reaction mixture was stirred at 75° C. for 4 hours. H₂O (5 mL) was added to the mixture. The solids were collected by filtration, washed with water and dried under vacuum to give 6-(4-(5-((7-bromo-8-methoxy-4-oxo-3,4-dihydrophthalazin-1-yl)methyl)-2-fluorobenzoyl)piperazin-1-yl)nicotinonitrile (55 mg, 93% yield). ¹H NMR (400 MHz, DMSO-d₆) δ 12.76 (s, 1H), 8.51 (d, J=2.2 Hz, 1H), 80) (d, J=8.5 Hz, 1H), 7.99 (d, J=8.5 Hz, 1H), 7.90 (dd, J=9.0, 2.3 Hz, 1H), 7.37-7.17 (m, 3H), 6.92 (d, J=9.0 Hz, 1H), 4.39 (s, 2H), 3.86 (s, 3H), 3.63 (dd, J=88.3, 44.8 Hz, 8H).

### Step 8

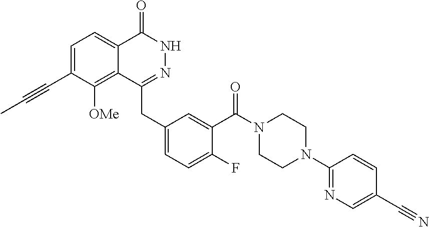 [SMILES: CC#Cc1ccc2c(=O)[nH]nc(Cc3ccc(F)c(C(=O)N4CCN(c5ccc(C#N)cn5)CC4)c3)c2c1OC]

### 6-(4-(2-Fluoro-5-((8-methoxy-4-oxo-7-(prop-1-yn-1-yl)-3,4-dihydrophthalazin-1-yl)methyl)benzoyl)piperazin-1-yl)nicotinonitrile

To a solution of 6-(4-(5-((7-bromo-8-methoxy-4-oxo-3,4-dihydrophthalazin-1-yl)methyl)-2-fluorobenzoyl)piperazin-1-yl)nicotinonitrile (45 mg, 77.94 μmol) in toluene (1 mL) was added tributyl(prop-1-ynyl)stannane (51.30 mg, 155.87 μmol), Pd(PPh₃)₄ (9.01 mg, 7.79 μmol) at 25° C. under N₂ (g). The reaction was stirred at 100° C. under microwave for 1 hour. The mixture was filtered and purified by prep-HPLC to give 6-[4-[2-fluoro-5-[(8-methoxy-4-oxo-7-prop-1-ynyl-3H-phthalazin-1-yl)methyl]benzoyl]piperazin-1-yl]pyridine-3-carbonitrile (7.65 mg, 18% yield) as a white solid. LCMS ESI m/z 537.2 [M+H]⁺. ¹H NMR (400 MHz, DMSO-d₆) δ 12.68 (s, 1H), 8.51 (d, J=2.0 Hz, 1H), 7.98 (d, J=8.2 Hz, 1H), 7.90 (dd, J=9.1, 2.4 Hz, 1H), 7.78 (d, J=8.2 Hz, 1H), 7.32-7.27 (m, 1H), 7.22 (dt, J=12.6, 5.9 Hz, 2H), 6.92 (d, J=9.1 Hz, 1H), 4.35 (s, 2H), 3.91 (s, 3H), 3.79-3.69 (m, 4H), 3.62 (s, 2H), 3.29 (s, 2f), 2.15 (s, 3H).

### Synthesis of Example 82: 6-(4-(3-((6-Fluoro-7-methoxy-4-oxo-3,4-dihydrophthalazin-1-yl)methyl)benzoyl)piperazin-1-yl)nicotinonitrile

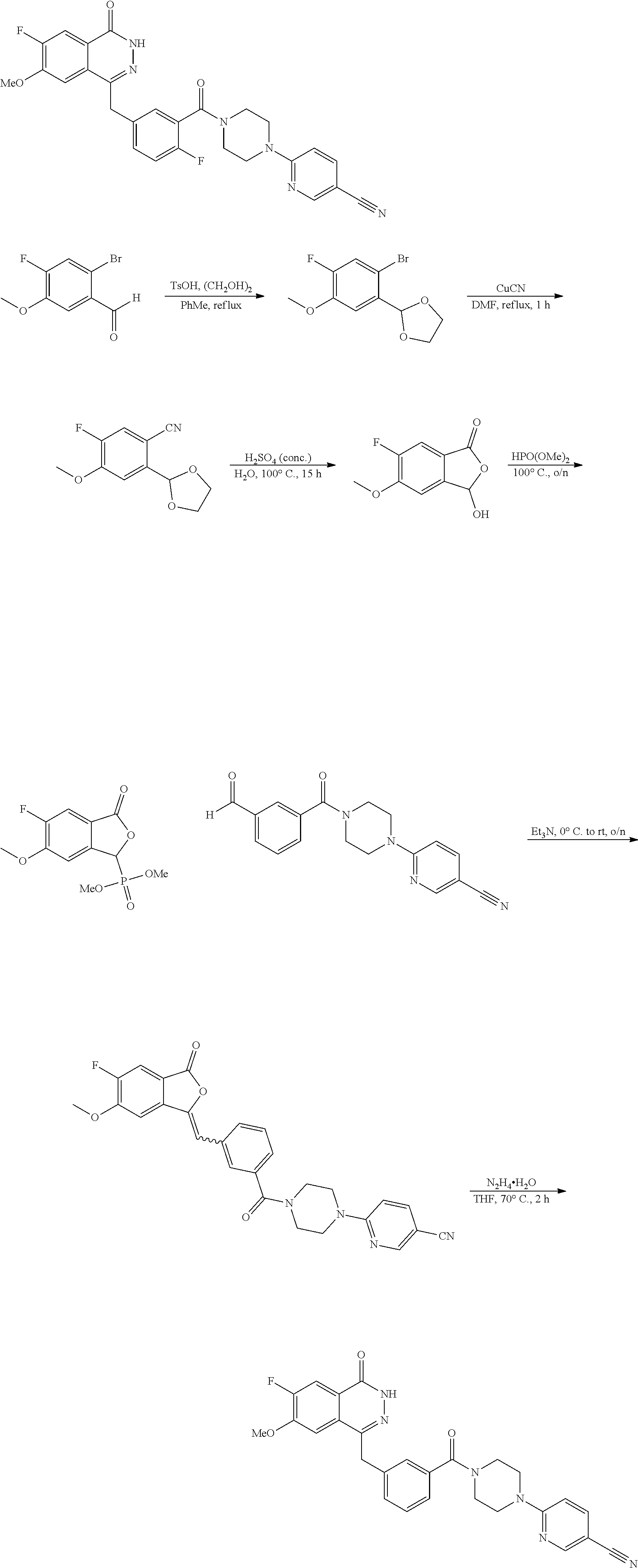

### Step 1

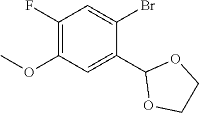

### 2-(2-Bromo-4-fluoro-5-methoxyphenyl)-1,3-dioxolane

To a solution of 2-bromo-4-fluoro-5-methoxybenzaldehyde (2.0 g, 8.6 mmol) and (CH₂OH)₂ (2.6 g, 43 mmol) in dry toluene (100 mL) was added TsOH (0.29 g, 0.17 mmol) at room temperature. The resulting mixture was allowed to heat to 120° C., and stirred at 120° C. for 24 hours. The mixture was diluted with EtOAc (100 mL). The solution was washed by a sat. aq of NaHCO₃ (100 mL×3). The organic layer was separated, dried, filtered and concentrated. The residue was purified by silica gel chromatography (petroleum ether/EtOAc=20/1 to 10/1, v/v) to give 2-(2-bromo-4-fluoro-5-methoxyphenyl)-1,3-dioxolane (2.2 g, 92% yield) as a white solid. ¹H NMR (40) MHz, CDCl₃) δ 7.54 (d, J=12.0 Hz, 1H), 7.21 (d, J=8.0 Hz, 1H), 5.83 (s, 1H), 4.09-4.05 (m, 2H), 3.95-3.92 (m, 2H), 3.81 (s, 3H).

### Step 2

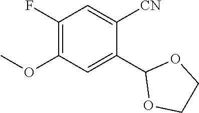 [SMILES: COc1cc(C2OCCO2)c(C#N)cc1F]

### 2-(1,3-Dioxolan-2-yl)-5-fluoro-4-methoxybenzonitrile

A slurry of 2-(2-bromo-4-fluoro-5-methoxyphenyl)-1,3-dioxolane (1.0 g, 3.6 mmol) and CuCN (0.33 g, 3.6 mmol) in dry DMF (20 mL) was stirred at 165° C. (oil bath) for 1 hour. The reaction mixture was poured into H₂O (50 mL), extracted with EA (30 mL×3). The combined organic layers were washed with brine (20 mL×3), dried, filtered and concentrated. The residue was purified by silica gel chromatography (petroleum ether/EtOAc=50/1 to 2/1, v/v) to give 2-(1,3-dioxolan-2-yl)-5-fluoro-4-methoxybenzonitrile (0.7 g, 88% yield) as an off-white solid. ¹H NMR (400 MHz, CDCl₃) δ 7.54 (d, J=12.0 Hz, 1H), 7.48 (d, J=8.0 Hz, 1H), 5.93 (s, 1H), 4.20-4.19 (m, 2H), 4.09-4.07 (m, 2H), 4.00 (m, 3H).

### Step 3

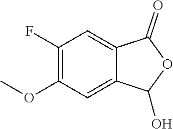

### 6-Fluoro-3-hydroxy-5-methoxyisobenzofuran-1(3H)-one

A slurry of solution of 2-(1,3-dioxolan-2-yl)-5-fluoro-4-methoxybenzonitrile (0.6 g, 2.6 mmol) in a mixture of conc. H₂SO₄ (8 mL) and H₂O (8 mL) was stirred at 100° C. for 15 hours. The reaction solution was added dropwise to a solution of sat. NaHCO₃ (200 mL). The solution was extracted with EtOAc (30 mL×4). The combined organic layers were dried, filtered, and concentrated. The residue was purified by silica gel chromatography (DCM/MeOH=40/1 to 20/1, v/v) to give the desired compound of 6-fluoro-3-hydroxy-5-methoxyisobenzofuran-1(3H)-one (0.37 g, 60% yield) as a yellow solid. ¹H NMR (400 MHz, DMSO-d₆) δ 7.57 (d, J=12.0 Hz, 1H), 6.88 (d, J=4.0 Hz, 1H), 6.54 (s, 1H), 3.89 (s, 3H).

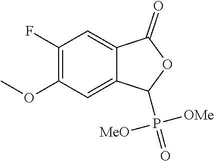 [SMILES: COc1cc2c(cc1F)C(=O)OC2P(=O)(OC)OC]

### Dimethyl 5-fluoro-6-methoxy-3-oxo-1,3-dihydroisobenzofuran-1-ylphosphonate

Following the procedure of step 5 in Example 1 but starting with 6-fluoro-3-hydroxy-5-methoxyisobenzofuran-1(3H)-one gave the title compound as a yellow oil. ¹H NMR (400 MHz, DMSO-d₆) δ 7.59 (d, J=12.0 Hz, 1H), 7.29-7.27 (m, 1H), 5.64 (d, J=12.0 Hz, 1H), 4.02 (s, 3H), 3.95 (d, J=12.0 Hz, 1H), 3.63 (d, J=8.0 Hz, 1H).

### Step 5

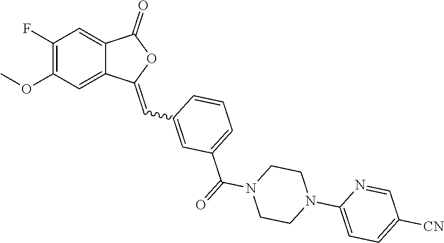 [SMILES: COc1cc2c(cc1F)C(=O)OC2=Cc1cccc(C(=O)N2CCN(c3ccc(C#N)cn3)CC2)c1]

### 6-(4-(3-((5-Fluoro-6-methoxy-3-oxoisobenzofuran-1(3H)-ylidene)methyl)benzoyl)piperazin-1-yl)nicotinonitrile

Following the procedure of step 6 in Example 1 but starting with dimethyl 5-fluoro-6-methoxy-3-oxo-1,3-dihydroisobenzofuran-1-ylphosphonate gave the title compound as a light yellow solid. LCMS ESI m/z: 485.1 [M+H]⁺.

### Step 6

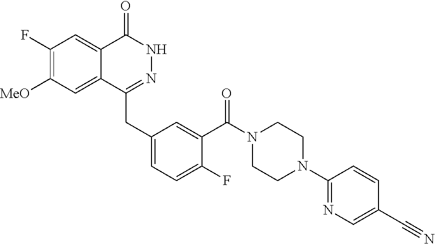 [SMILES: COc1cc2c(Cc3ccc(F)c(C(=O)N4CCN(c5ccc(C#N)cn5)CC4)c3)n[nH]c(=O)c2cc1F]

### 6-(4-(3-((6-Fluoro-7-methoxy-4-oxo-3,4-dihydrophthalazin-1-yl)methyl)benzoyl)piperazin-1-yl)nicotinonitrile

Following the procedure of step 7 in Example 1, but starting with 6-(4-(3-((5-fluoro-6-methoxy-3-oxoisobenzofuran-1(3H)-ylidene)methyl)benzoyl)piperazin-1-yl)nicotinonitrile gave the title compound as a white solid. LCMS ESI m/z: 498.8 [M+H]⁺. ¹H NMR (400 MHz, DMSO-d₆) δ 12.59 (s, 1H), 8.51 (d, J=4.0 Hz, 1H), 7.93 (d, J=4.0 Hz, 1H), 7.89 (dd, J=4.0 Hz, J=8.0 Hz, 1H), 7.52 (d, J=8.0 Hz, 1H), 7.46-7.38 (m, 3H), 7.30 (d, J=8.0 Hz, 1H), 6.32 (d, J=12.0 Hz, 1H), 4.38 (s, 2H), 3.97 (s, 3H), 3.68-3.59 (m, 6H), 3.41-3.40 (m, 2H).

### Synthesis of Example 83: 6-(4-(2-Fluoro-5-((6-methyl-4-oxo-7-(prop-1-yn-1-yl)-3,4-dihydrophthalazin-1-yl)methyl)benzoyl)piperazin-1-yl)nicotinonitrile

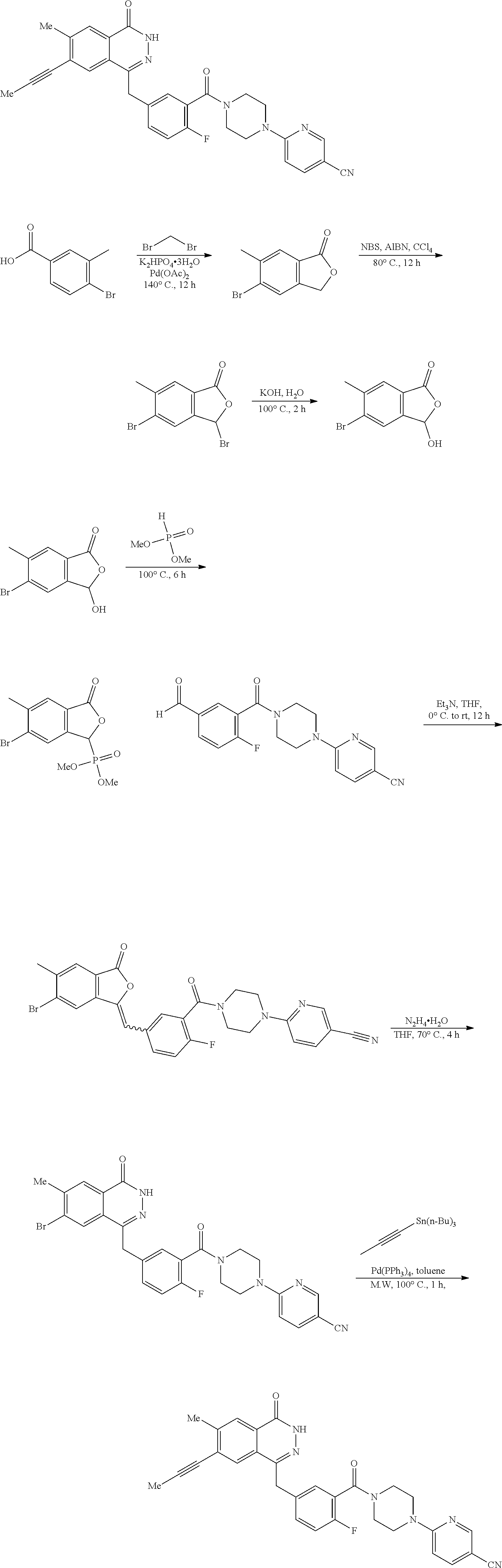

### Step 1

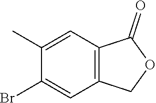

### 5-Bromo-6-methyl-3H-isobenzofuran-1-one

4-bromo-3-methyl-benzoic acid (5 g, 23.25 mmol), dibromomethane (4.04 g, 23.25 mmol) and K₂HPO₄·3H₂O (15.92 g, 69.75 mmol) were added to a 100 mL sealed tube. The mixture was purged with N₂ (g) for 2 times, then Pd(OAc)₂ (522.01 mg, 2.33 mmol) was added. The resultant mixture was stirred at 140° C. for 12 hours. After the reaction mixture was cooled to room temperature, the content was diluted with CH₂Cl₂ (20 mL), filtered through a celite pad. The organic layer was washed with H₂O (20 mL), brine (20 mL), dried over Na₂SO₄, concentrated in vacuum to afford the crude product, which was purified by silica gel chromatography (petroleum ether:EtOAc=10:1 to 5:1) to afford 5-bromo-6-methyl-3H-isobenzofuran-1-one (3.7 g, 70% yield) as a white solid. ¹H NMR (400 MHz, CDCl₃) δ 7.77 (s, 1H), 7.70 (s, 1H), 5.26 (s, 2H), 2.51 (s, 3H).

### Step 2

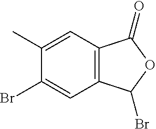

### 3,5-Dibromo-6-methyl-3H-isobenzofuran-1-one

5-Bromo-6-methyl-3H-isobenzofuran-1-one (3.7 g, 16.30 mmol), NBS (3.19 g, 17.93 mmol, 1.52 mL), AIBN (267.59 mg, 1.63 mmol) and CCl₄ (40 mL) were added to a 100 mL bottled flask. The mixture was stirred at 80° C. for 12 hours. The mixture was diluted with EtOAc (40 mL), and washed with H₂O (20 mL), brine (30 mL), dried over Na₂SO₄, concentrated in vacuum to afford the crude product, which was purified by silica gel chromatography (petroleum ether:EtOAc=10:1 to 5:1) to afford 3,5-dibromo-6-methyl-3H-isobenzofuran-1-one (2 g, 40% yield) as a white solid. ¹H NMR (400 MHz, CDCl₃) δ 7.82 (s, 1H), 7.77 (s, 1H), 7.33 (s, 1H), 2.54 (s, 3H)

### Step 3

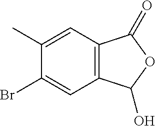

### 5-Bromo-3-hydroxy-6-methylisobenzofuran-1(3H)-one

3,5-Dibromo-6-methyl-3H-isobenzofuran-1-one (2 g, 6.54 mmol). KOH (733.53 mg, 13.07 mmol, 358.87 μL) and water (20 mL) were added to a 250 mL bottled flask. The mixture was stirred at 100° C. for 2 hours. The mixture was diluted in EtOAc (20 mL), the organic layer was washed with H₂O (10 mL), brine (30 mL), dried over Na₂SO₄, concentrated in vacuum to afford the crude product, which was purified by silica gel chromatography (petroleum ether:EtOAc=10:1 to 5:1) to afford 5-bromo-3-hydroxy-6-methylisobenzofuran-1(3H)-one (1.5 g, 94.4% yield) as a white solid. ¹H NMR (400 MHz, DMSO-d₆) δ 8.30 (d, J=7.6 Hz, 1H), 7.97 (s, 1H), 7.88 (s, 1H), 6.68 (d, J=5.5 Hz, 1H), 2.52 (s, 3H).

### Step 4

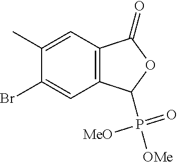 [SMILES: COP(=O)(OC)C1OC(=O)c2cc(C)c(Br)cc21]

### Dimethyl (6-bromo-5-methyl-3-oxo-1,3-dihydroisobenzofuran-1-yl)phosphonate

5-Bromo-3-hydroxy-6-methyl-3H-isobenzofuran-1-one (1.5 g, 6.17 mmol) was added into dimethylphosphite (679.16 mg, 6.17 mmol, 5 mL). The resulting mixture was stirred at 100° C. for 6 hours. The mixture was quenched with H₂O (20 mL), extracted with EtOAc (20 mL×2). The combined organic phase was washed with H₂O (20 mL×2), dried over Na₂SO₄, filtered and concentrated under reduced pressure to afford the crude product, which was purified by silica gel chromatography (petroleum ether:EtOAc=20:1 to 1:1) to afford dimethyl (6-bromo-5-methyl-3-oxo-1,3-dihydroisobenzofuran-1-yl)phosphonate (850 mg, 41% yield) as a white solid. ¹H NMR (400 MHz, CDCl₃) δ 7.94 (s, 1H), 7.79 (s, 1H), 5.64 (d, J=10.9 Hz, 1H), 3.93 (d, J=10.9 Hz, 3H), 3.68 (d, J=10.7 Hz, 3H), 2.52 (s, 3H).

### Step 5

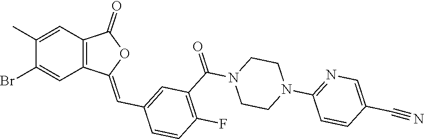 [SMILES: Cc1cc2c(cc1Br)/C(=C/c1ccc(F)c(C(=O)N3CCN(c4ccc(C#N)cn4)CC3)c1)OC2=O]

### 6-(4-(5-((6-bromo-5-methyl-3-oxoisobenzofuran-1(3H)-ylidene)methyl)-2-fluorobenzoyl)piperazin-1-yl)nicotinonitrile

To a mixture of dimethyl (6-bromo-5-methyl-3-oxo-1,3-dihydroisobenzofuran-1-yl)phosphonate (740 mg, 2.21 mmol) in THF (10 mL) was added 6-[4-(2-fluoro-5-formyl-benzoyl)piperazin-1-yl]pyridine-3-carbonitrile (1.34 g, 3.98 mmol) and Et₃N (670.39 mg, 6.63 mmol, 923.41 μL). The resulting mixture was stirred at 25° C. for 12 hours. The mixture was quenched with sat. NaHSO₃ solution (20 mL) and filtered. The filter cake was washed with H₂O (20 mL×2) and dried under reduced pressure to give 6-(4-(5-((6-bromo-5-methyl-3-oxoisobenzofuran-1(3H)-ylidene)methyl)-2-fluorobenzoyl)piperazin-1-yl)nicotinonitrile (1.36 g, crude) as a yellow solid.

### Step 6

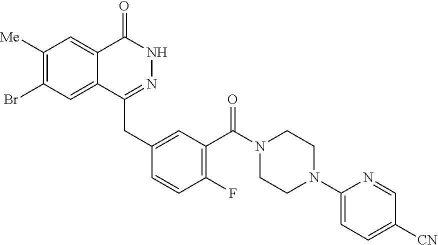 [SMILES: Cc1cc2c(=O)[nH]nc(Cc3ccc(F)c(C(=O)N4CCN(c5ccc(C#N)cn5)CC4)c3)c2cc1Br]

### 6-[4-[5-[(7-Bromo-6-methyl-4-oxo-3H-phthalazin-1-yl)methyl]-2-fluoro-benzoyl]piperazin-1-yl]pyridine-3-carbonitrile

To a mixture of 6-(4-(5-((6-bromo-5-methyl-3-oxoisobenzofuran-1(3H)-ylidene)methyl)-2-fluorobenzoyl)piperazin-1-yl)nicotinonitrile (1.36 g, 2.48 mmol) in THF (20 mL) was added hydrazine hydrate (310.95 mg, 4.97 mmol, 302.77 μL). The resulting mixture was stirred at 70° C. for 4 hours. The mixture was cooled to rt, and filtered. The filter cake was washed with a solution of petrol ether/EtOAc=10/1 (20 mL×2). The solid was dried under reduced pressure to give 6-[4[5-[(7-bromo-6-methyl-4-oxo-3H-phthalazin-1-yl)methyl]-2-fluoro-benzoyl]piperazin-1-yl]pyridine-3-carbonitrile (1.1 g, 79% yield) as a yellow solid. ¹H NMR (400 MHz, DMSO-d₆) δ 12.68 (s, 1H), 8.57 (d, J=2.2 Hz, 1H), 8.24 (d, J=16.4 Hz, 2H), 7.96 (dd, J=9.1, 2.3 Hz, 1H), 7.54-7.41 (m, 2H), 7.32 (t, J=9.0 Hz, 1H), 6.98 (d, J=9.1 Hz, 1H), 4.39 (s, 2H), 3.80 (d, J=7.9 Hz, 4H), 3.66 (s, 2H), 3.37 (d, J=2.4 Hz, 2H), 2.59 (s, 3H).

### Step 7

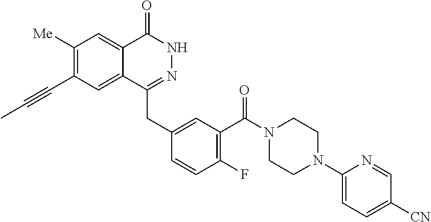 [SMILES: CC#Cc1cc2c(Cc3ccc(F)c(C(=O)N4CCN(c5ccc(C#N)cn5)CC4)c3)n[nH]c(=O)c2cc1C]

### 6-(4-(2-Fluoro-5-((6-methyl-4-oxo-7-(prop-1-yn-1-yl)-3,4-dihydrophthalazin-1-yl)methyl)benzoyl)piperazin-1-yl)nicotinonitrile

6-[4-[5-[(7-Bromo-6-methyl-4-oxo-3H-phthalazin-1-yl)methyl]-2-fluoro-benzoyl]piperazin-1-yl]pyridine-3-carbonitrile (100 mg, 178.12 μmol), tributyl(prop-1-ynyl)stannane (117.24 mg, 356.25 μmol), palladium;triphenylphosphane (20.58 mg, 17.81 μmol) and toluene (2 mL) were added to a 10 mL sealed tube. The mixture was purged with N₂ (g) for 3 times, the resultant mixture was stirred at 100° C. for 1 hr. The mixture was concentrated under reduced pressure to give the crude product, which was purified by prep-HPLC to afford 6-(4-(2-fluoro-5-((6-methyl-4-oxo-7-(prop-1-yn-1-yl)-3,4-dihydrophthalazin-1-yl)methyl)benzoyl)piperazin-1-yl)nicotinonitrile (17.8 mg, 19% yield) as a white solid. LCMS ESI 520.8 [M+H]⁺. ¹H-NMR (400 MHz, DMSO-d₆) δ 12.53 (s, 1H), 8.51 (d, J=2.3 Hz, 1H), 8.12 (s, 1H), 7.90 (dd, J=9.0, 2.3 Hz, 2H), 7.43-7.37 (m, 2H), 7.25 (t, J=8.9 Hz, 1H), 6.92 (d, J=9.1 Hz, 1H), 4.31 (s, 2H), 3.74 (d, J=9.4 Hz, 4H), 3.61 (s, 2H), 3.30-3.24 (m, 2H), 2.51 (d, J=1.4 Hz, 3H), 2.13 (s, 3H).

### Synthesis of Example 84: 6-(4-(2-Fluoro-5-((4-oxo-7-(prop-1-yn-1-yl)-5-(trifluoromethyl)-3,4-dihydrophthalazin-1-yl)methyl)benzoyl)piperazin-1-yl)nicotinonitrile

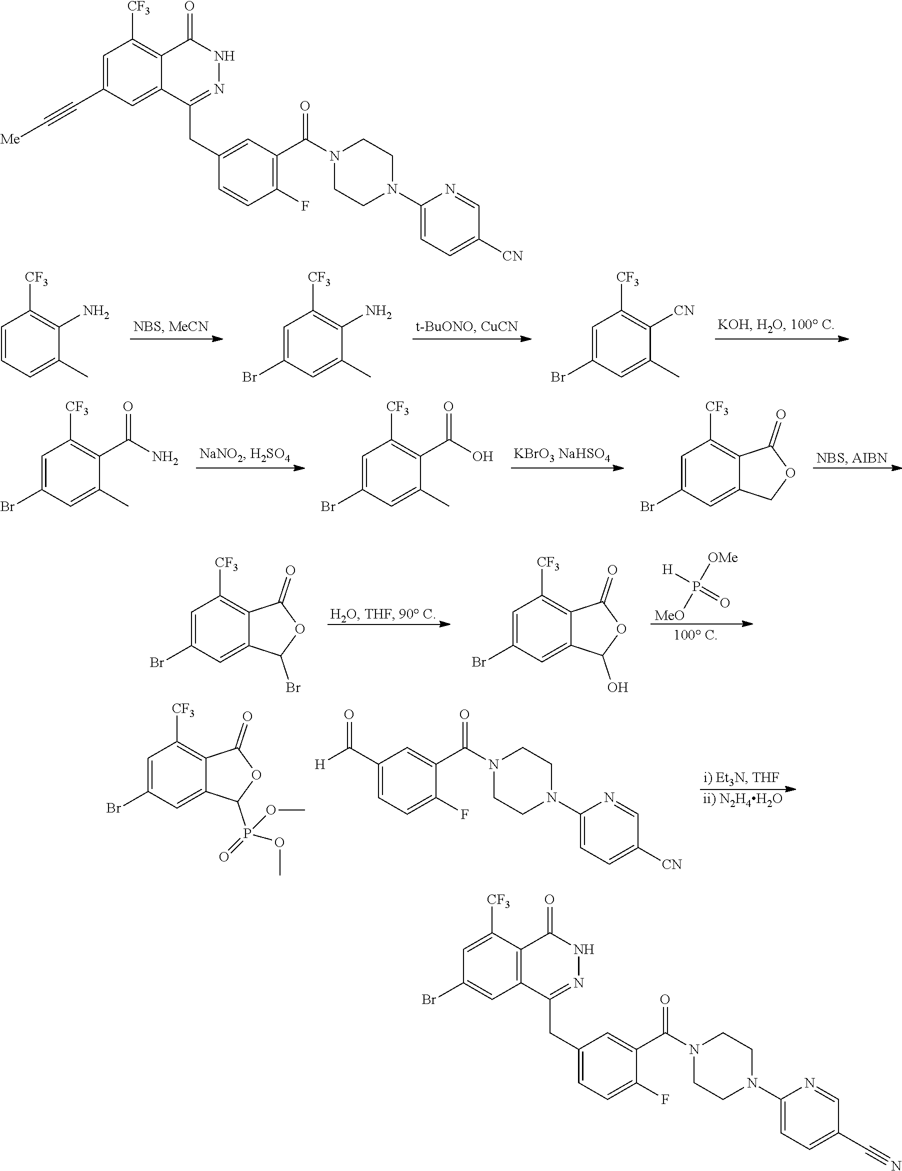

### Step 1

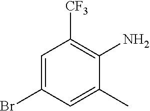

### 4-Bromo-2-methyl-6-(trifluoromethyl)aniline

To a solution of 2-methyl-(trifluoromethyl)aniline (5.00 g, 28.60 mmol) in MeCN (20 mL) was added NBS (5.09 g, 28.6 mmol) in portions. The reaction mixture was stirred at rt for 3 hours, then diluted with EtOAc (100 mL) and water (100 mL). The organic phase was separated and the aqueous phase was extracted with EtOAc (50 mL×2). The combined organic layer was dried over Na₂SO₄, filtered and concentrated in vacuo. The residue was purified by silica gel chromatography (EtOAc/petroleum ether=0 to 10%) to afford 4-bromo-2-methyl-6-(trifluoromethyl)aniline as a yellow oil (6.90 g, 95% yield). LCMS ESI m/z 254 [M+H]⁺.

### 4-Bromo-2-methyl-6-(trifluoromethyl)benzonitrile

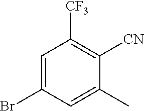

A mixture of 4-bromo-2-methyl-6-(trifluoromethyl)aniline (6.90 g, 27.27 mmol), tert-Butyl nitrite (14.05 g, 136.36 mmol) and CuCN (3.83 g, 40.91 mmol) in DMSO (100 mL) were stirred at 70° C. under Ar (g) for 1 hour. The reaction mixture was cooled to rt, diluted with water (50 mL) and extracted with EtOAc (3×50 mL). The combined organic layer was dried over Na₂SO₄, filtered and concentrated in vacuo. The residue was purified by silica gel chromatography (EtOAc/petroleum ether=10 to 20%) to afford 4-bromo-2-methyl-6-(trifluoromethyl)benzonitrile as a yellow oil (3.62 g, 50% yield). LCMS ESI m/z 264 [M+H]⁺.

### 4-Bromo-2-methyl-6-(trifluoromethyl)benzamide

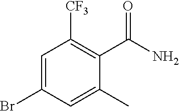

A mixture of 4-bromo-2-methyl-6-(trifluoromethyl)benzonitrile (3.62 g, 13.76 mmol) and KOH (15.42 g, 275.29 mmol) in H₂O (50 mL) were stirred at 10) ° C. for 16 hours. The mixture was cooled to rt, then extracted with EtOAc (3×50 mL). The combined organic layer was dried over Na₂SO₄, filtered and concentrated in vacuo. The residue was purified by silica gel chromatography (EtOAc/petroleum ether=30 to 50%) to give 4-bromo-2-methyl-6-(trifluoromethyl)benzamide as a yellow solid (2.32 g, 60% yield). LC-MS ESI m/z 282 [M+H]⁺.

### Step 4

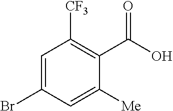 [SMILES: Cc1cc(Br)cc(C(F)(F)F)c1C(=O)O]

### 4-Bromo-2-methyl-6-(trifluoromethyl)benzoic acid

To a mixture of 4-bromo-2-methyl-6-(trifluoromethyl)benzamide (2.32 g, 8.26 mmol) in H₂SO₄ (40 mL) was added a solution of NaNO₂ (9.68 g, 140.36 mmol) in H₂O (15 mL) dropwise at 0° C. After stirred at 0° C. for 30 mins, the reaction mixture was heated to 130° C. for 14 hours. The mixture was cooled to rt, diluted with H₂O (40 mL) and extracted with EtOAc (3×40 mL). The combined organic layer was dried over Na₂SO₄, filtered and concentrated in vacuo. The residue was purified by silica gel chromatography (EtOAc/pet ether=50/o to 80%) to give 4-bromo-2-methyl-6-(trifluoromethyl)benzoic acid as a yellow solid (2.11 g, 91% yield). LCMS ESI m/z 265, [M−H₂O+H]⁺.

### Step 5

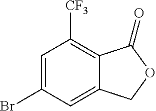 [SMILES: O=C1OCc2cc(Br)cc(C(F)(F)F)c21]

### 5-Bromo-7-(trifluormethyl)isobenzofuran-1(3H)-one

A mixture of 4-bromo-2-methyl-6-(trifluoromethyl)benzoic acid (2.11 g, 7.48 mmol), KBrO3 (7.49 g, 44.89 mmol) and NaHSO3 (4.67 g, 44.89 mmol) in H2O (50 mL) were stirred at rt for 5 days.

The reaction mixture was extracted with EtOAc (3×50 mL). The combined organic layer was dried over Na2SO4, filtered and concentrated in vacuo. The residue was purified by silica gel chromatography (EtOAc/pet ether=0 to 10%) to give 5-bromo-7-(trifluoromethyl)isobenzofuran-1(3H)-one as a yellow solid (1.26 g, 60% yield). LCMS ESI m/z 281 [M+H]+, 1H NMR (400 MHz, CDCl3) δ 7.97 (s, 1H), 7.68 (s, 1H), 5.33 (s, 2H).

### Step 6

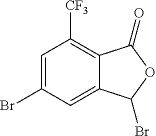 [SMILES: O=C1OC(Br)c2cc(Br)cc(C(F)(F)F)c21]

### 3,5-Dibromo-7-(trifluoromethyl)isobenzofuran-1(3H)-one

A mixture of 5-bromo-7-(trifluoromethyl)isobenzofuran-1(3H)-one (1.26 g, 4.50 mmol). AIBN (148 mg, 0.90 mmol) and NBS (960 mg, 5.40 mmol) in CCl4 (20 mL) were stirred at 80° C. for 8 hours. The mixture was concentrated in vacuo, and the residue was purified by silica gel chromatography (EtOAc/pet ether=0 to 10%) to give 3,5-dibromo-7-(trifluoromethyl)isobenzofuran-1(3H)-one as a yellow solid (880 mg, 55% yield). LCMS ESI m/z 359 [M+H]+.

### Step 7

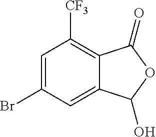 [SMILES: O=C1OC(O)c2cc(Br)cc(C(F)(F)F)c21]

### 5-Bromo-3-hydroxy-7-(trifluoromethyl)isobenzofuran-1(3H)-one

A solution of 3,5-dibromo-7-(trifluoromethyl)isobenzofuran-1(3H)-one (880 mg, 2.46 mmol) in H2O (10 mL) and THF (10 mL) was stirred at 90° C. for 6 hours. The reaction mixture was cooled to rt, then extracted with EtOAc (3×30 mL). The combined organic layer was dried over Na2SO4, filtered and concentrated in vacuo. The residue was purified by silica gel chromatography (EtOAc/petroleum ether=30 to 40%) to give 5-bromo-3-hydroxy-7-(trifluoromethyl)isobenzofuran-1(3H)-one (470 mg, 65% yield). LCMS ESI m/z 297 [M+H]+.

### Step 8

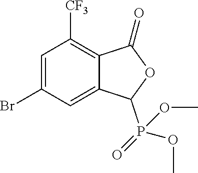 [SMILES: COP(=O)(OC)C1OC(=O)c2c1cc(Br)cc2C(F)(F)F]

### Dimethyl 6-bromo-3-oxo-4-(trifluoromethyl)-1,3-dihydroisobenzofuran-1-ylphosphonate

A mixture of 5-bromo-3-hydroxy-7-(trifluoromethyl)isobenzofuran-1(3H)-one (470 mg, 1.59 mmol) in dimethyl phosphonate (5 mL) was stirred at 100° C. for 12 hours. The mixture was cooled to rt, diluted with H2O (20 mL) and extracted with EtOAc (3-20 mL). The combined organic layer was dried over Na2SO4, filtered and concentrated in vacuo. The residue was purified by silica gel chromatography (EtOAc/petroleum ether=30 to 40%) to give dimethyl 6-bromo-3-oxo-4-(trifluoromethyl)-1,3-dihydroisobenzofuran-1-ylphosphonate as a yellow oil (380 mg, 63% yield). LCMS ESI m/z 389 [M+H]+.

### Step 9

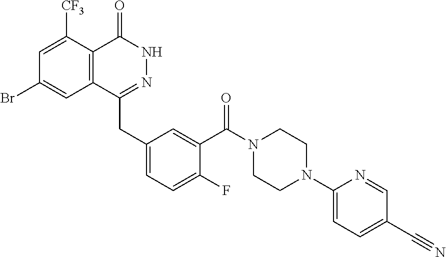 [SMILES: N#Cc1ccc(N2CCN(C(=O)c3cc(Cc4n[nH]c(=O)c5c(C(F)(F)F)cc(Br)cc45)ccc3F)CC2)nc1]

### 6-(4-(5-((7-Bromo-4-oxo-5-(trifluoromethyl)-3,4-dihydrophthalazin-1-yl)methyl)-2-fluorobenzoyl)piperazin-1-yl)nicotinonitrile

A mixture of 6-bromo-3-oxo-4-(trifluoromethyl)-1,3-dihydroisobenzofuran-1-ylphosphonate (380 mg, 0.98 mmol), 6-(4-(2-fluoro-5-formylbenzoyl)piperazin-1-yl)nicotinonitrile (330 mg, 0.98 mmol) and Et3N (300 mg, 3 mmol) in THF (7 mL) was stirred at rt for 12 hours. To the reaction mixture was added N2H4·H2O (353 mg, 6 mmol), then stirred at 70° C. for 2 hours. The reaction mixture was concentrated in vacuo. The solids were diluted with MeOH (5 mL), filtered and dried under vacuum to give 6-(4-(5-((7-bromo-4-oxo-5-(trifluoromethyl)-3,4-dihydrophthalazin-1-yl)methyl)-2-fluorobenzoyl)piperazin-1-yl)nicotinonitrile as a white solid (150 mg, 25% yield). This crude product was used in the next step without further purification. LCMS ESI m/z 615 [M+H]+.

### Step 10

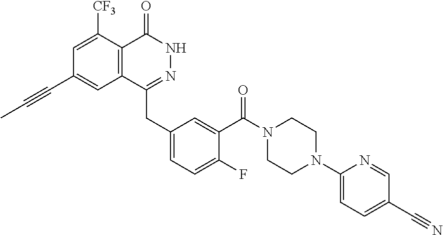 [SMILES: CC#Cc1cc(C(F)(F)F)c2c(=O)[nH]nc(Cc3ccc(F)c(C(=O)N4CCN(c5ccc(C#N)cn5)CC4)c3)c2c1]

### 6-(4-(2-Fluoro-5-((4-oxo-7-(prop-1-ynyl)-5-(trifluoromethyl)-3,4-dihydrophthalazin-1-yl)methyl)benzoyl)piperazin-1-yl)nicotinonitrile

A mixture of 6-(4-(5-((7-bromo-4-oxo-5-(trifluoromethyl)-3,4-dihydrophthalazin-1-yl)methyl)-2-fluorobenzoyl)piperazin-1-yl)nicotinonitrile (150 mg, 0.25 mmol), tributyl(prop-1-ynyl)stannane (330 mg, 1.00 mmol) and Pd(dppf)Cl2 (22 mg, 0.03 mmol) in 1,4-dioxane (8 mL) was stirred at 100° C. under Ar (g) for 10 hours. The reaction mixture was cooled to rt, diluted with water (20 mL) and extracted with EtOAc (3×20 mL). The combined organic layer was dried over Na2SO4, filtered and concentrated in vacuo. The residue was purified by silica gel chromatography (EtOAc/petroleum ether=80 to 100%) to give the desired product as a white solid (80 mg, 57% yield). LCMS ESI m/z 575 [M+H]+. 1H NMR (400 MHz, DMSO-d6) δ 12.79 (s, 1H), 8.51 (d, J=2.0 Hz, 1H), 8.10 (s, 1H), 7.90 (dd, J=9.1, 2.3 Hz, 1H), 7.44-7.39 (m, 2H), 7.26 (t, J=9.3 Hz, 1H), 6.92 (d, J=9.2 Hz, 1H), 4.38 (s, 2H), 3.76 (d, J=8.3 Hz, 4H), 3.61 (s, 2H), 3.32-7.29 (m, 2H), 2.11 (s, 3H).

### Synthesis of Example 85: 6-(4-(5-((5-chloro-4-oxo-7-(prop-1-yn-1-yl)-3,4-dihydrophthalazin-1-yl)methyl)-2-fluorobenzoyl)piperazin-1-yl)nicotinonitrile

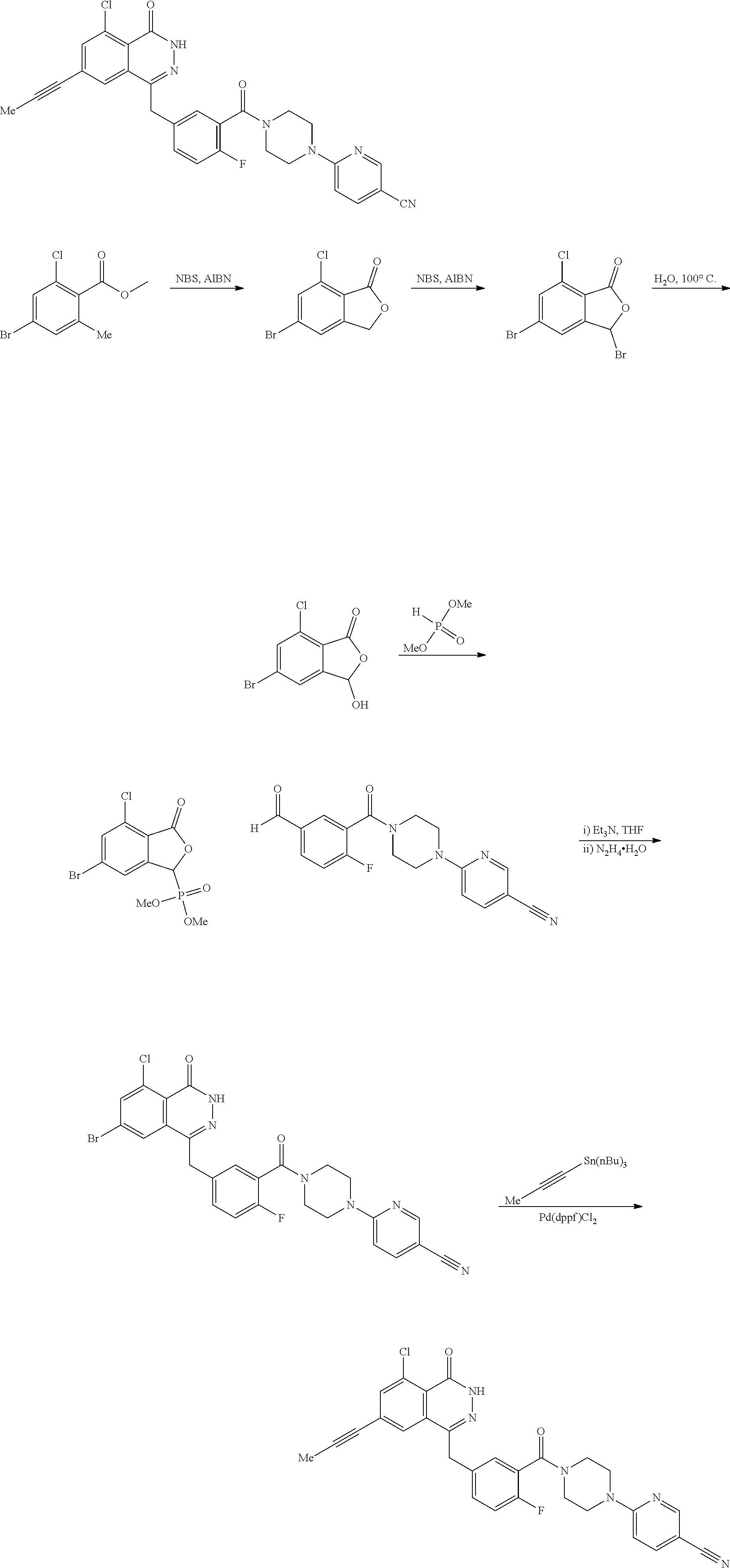

### Step 1

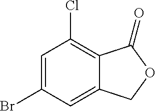 [SMILES: O=C1OCc2cc(Br)cc(Cl)c21]

### 5-Bromo-7-chloroisobenzofuran-1(3H)-one

A mixture of methyl 4-bromo-2-chloro-6-methylbenzoate (2.01 g, 7.59 mmol). NBS (6.75 g, 37.95 mmol) and AIBN (1.25 g, 7.59 mmol) in CCl₄ (100 mL) was stirred at 80° C. for 18 hours. The mixture was cooled to rt, diluted with H₂O (50 mL), and extracted with DCM (50 mL×3). The combined organic layer was dried over anhydrous Na₂SO₄, filtered and concentrated in vacuo. The residue was purified by silica gel chromatography (EtOAc/petroleum ether=0 to 10%) to obtain 5-bromo-7-chloroisobenzofuran-1(3H)-one as a white solid (1.41 g, 75% yield). LCMS ESI m/z 267 [M+H]⁺. ¹H NMR (400 MHz, CDCl₃) δ 7.66 (s, 1H), 7.56 (s, 1H), 5.25 (s, 2H).

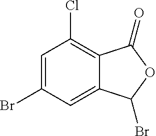 [SMILES: O=C1OC(Br)c2cc(Br)cc(Cl)c21]

### 3,5-Dibromo-7-chloroisobenzofuran-1(3H)-one

A mixture of methyl 5-bromo-7-chloroisobenzofuran-1(3H)one (1.41 g, 5.70 mmol), NBS (1.12 g, 6.27 mmol) and AIBN (187 mg, 1.14 mmol) in CCl₄ (30 mL) was stirred at 80° C. for 15 hours. The mixture was cooled to rt, diluted with H₂O (30 mL), and extracted with DCM (50 mL×3). The combined organic layer dried over anhydrous Na₂SO₄, filtered and concentrated in vacuo. The residue was purified by silica gel chromatography (EtOAc/pet ether=0 to 10%) to afford 3,5-dibromo-7-chloroisobenzofuran-1(3H)-one as a white solid (1.16 g, 62% yield). LCMS ESI m/z 326 [M+H]⁺.

### Step 3

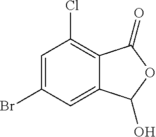 [SMILES: O=C1OC(O)c2cc(Br)cc(Cl)c21]

### 5-Bromo-7-chloro-3-hydroxyisobenzofuran-1(3H)-one

A mixture of 3,5-dibromo-7-chloroisobenzofuran-1(3H)-one (1.16 g, 3.55 mmol) in H₂O (25 mL) was stirred at 100° C. for 4 hours. The reaction was cooled to rt and extracted with DCM (50 mL×3). The combined organic layer was dried over anhydrous Na₂SO₄, filtered and concentrated in vacuo to give 5-bromo-7-chloro-3-hydroxyisobenzofuran-1(3H)-one as a light yellow oil (553 mg, 59% yield). LCMS ESI m/z 244 [M−H₂O+H]⁺.

### Step 4

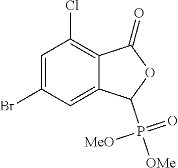 [SMILES: COP(=O)(OC)C1OC(=O)c2c(Cl)cc(Br)cc21]

### Dimethyl 6-bromo-4-chloro-3-oxo-1,3-dihydroisobenzofuran-1-ylphosphonate

A solution of 5-bromo-7-chloro-3-hydroxyisobenzofuran-1(3H)-one (658 mg, 2.50 mmol) in dimethylphosphite (10 mL) was heated to 100° C. for 2 hours. The reaction mixture was cooled to rt, diluted with H₂O (10 mL) and extracted with EtOAc (20 mL×3). The combined organic layer was washed with brine, dried over anhydrous Na₂SO₄, filtered and concentrated in vacuo to give dimethyl 6-bromo-4-chlor-3-oxo-1,3-dihydroisobenzofuran-1-ylphosphonate as a light yellow oil (649 mg, 73% yield). LCMS ESI m/z 355 [M+H]⁺.

### Step 5

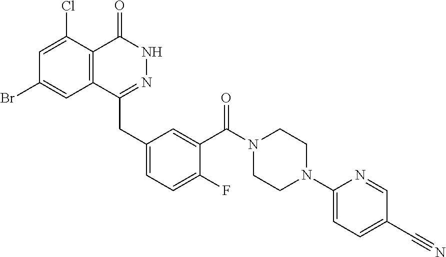 [SMILES: N#Cc1ccc(N2CCN(C(=O)c3cc(Cc4n[nH]c(=O)c5c(Cl)cc(Br)cc45)ccc3F)CC2)nc1]

### 6-(4-(5-((7-Bromo-5-chloro-4-oxo-3,4-dihydrophthalazin-1-yl)methyl)-2-fluorobenzoyl)piperazin-1-yl)nicotinonitrile

A solution of 6-(4-(2-fluoro-5-formylbenzoyl)piperazin-1-yl)nicotinonitrile (649 mg, 1.92 mmol), dimethyl 6-bromo-4-chloro-3-oxo-1,3-dihydroisobenzofuran-1-ylphosphonate (649 mg, 1.83 mmol) and Et₃N (554 mg, 5.48 mmol) in anhydrous THF (10 mL) was stirred at 25° C. under Ar (g) for 15 hours. To the reaction solution, hydrazine hydrate (914 mg, 18.26 mmol) was added and stirred at 70° C. for 1 hour. The reaction was cooled to rt, then THF was removed under vacuum. The solids were filtered, washed with water (10 mL) and petroleum ether/EtOAc (2:1, 10 mL) to give 6-(4-(54(7-bromo-5-chloro-4-oxo-3,4-dihydrophthalazin-1-yl)methyl)-2-fluorobenzoyl)piperazin-1-yl)nicotinonitrile as a white solid (135 mg, 12% yield). LCMS ESI m/z 581 [M+H]⁺.

### Step 6

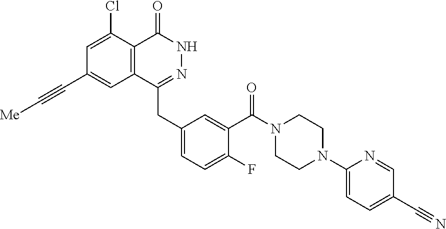 [SMILES: CC#Cc1cc(Cl)c2c(=O)[nH]nc(Cc3ccc(F)c(C(=O)N4CCN(c5ccc(C#N)cn5)CC4)c3)c2c1]

### 6-(4-(5-((5-Chloro-4-oxo-7-(prop-1-ynyl)-3,4-dihydrophthalazin-1-yl)methyl)-2-fluorobenzoyl)piperazin-1-yl)nicotinonitrile

A mixture of tributyl(prop-1-ynyl)stannane (183 mg, 0.56 mmol), 6-(4-(5-(7-bromo-5-chloro-4-oxo-3,4-dihydrophthalazin-1-yl)methyl)-2-fluorobenzoyl)piperazin-1-yl)nicotinonitrile (100 mg, 0.19 mmol) and Pd(dppf)Cl₂ (15 mg, 0.02 mmol) in 1,4-dioxane (5 mL) was stirred at 100° C. under Ar (g) for 5 hours. The reaction mixture was concentrated in vacuo and the residue was purified by silica gel chromatography (MeOH/DCM=0 to 5%) to afford 6-(4-(5-((5-chloro-4-oxo-7-(prop-1-ynyl)-3,4-dihydrophthalazin-1-yl)methyl)-2-fluorobenzoyl)piperazin-1-yl)nicotinonitrile as a white solid (26 mg, 28% yield). LCMS ESI m/z 541 [M+H]⁺. ¹H NMR (400 MHz, CDCl₃) δ 9.75 (s, 1H), 8.42 (d, J=1.9 Hz, 1H), 7.69 (d, J=1.3 Hz, 1H), 7.66 (dd, J=9.0, 2.3 Hz, 1H), 7.56 (d, J=1.3 Hz, 1H), 7.35-7.27 (m, 2H). 7.08 (t, J=8.8 Hz, 1H), 6.62 (d, J=9.1 Hz, 1H), 4.20 (s, 2H), 3.90 (s, 2H), 3.78 (s, 2H), 3.69 (t, J=5.2 Hz, 2H), 3.43 (s, 2H), 2.08 (s, 3H).

### Synthesis of Example 86: 6-(4-(2-Fluoro-5-((8-methyl-4-oxo-7-(prop-1-ynyl)-3,4-dihydrophthalazin-1-yl)methyl)benzoyl)piperazin-1-yl)nicotinonitrile

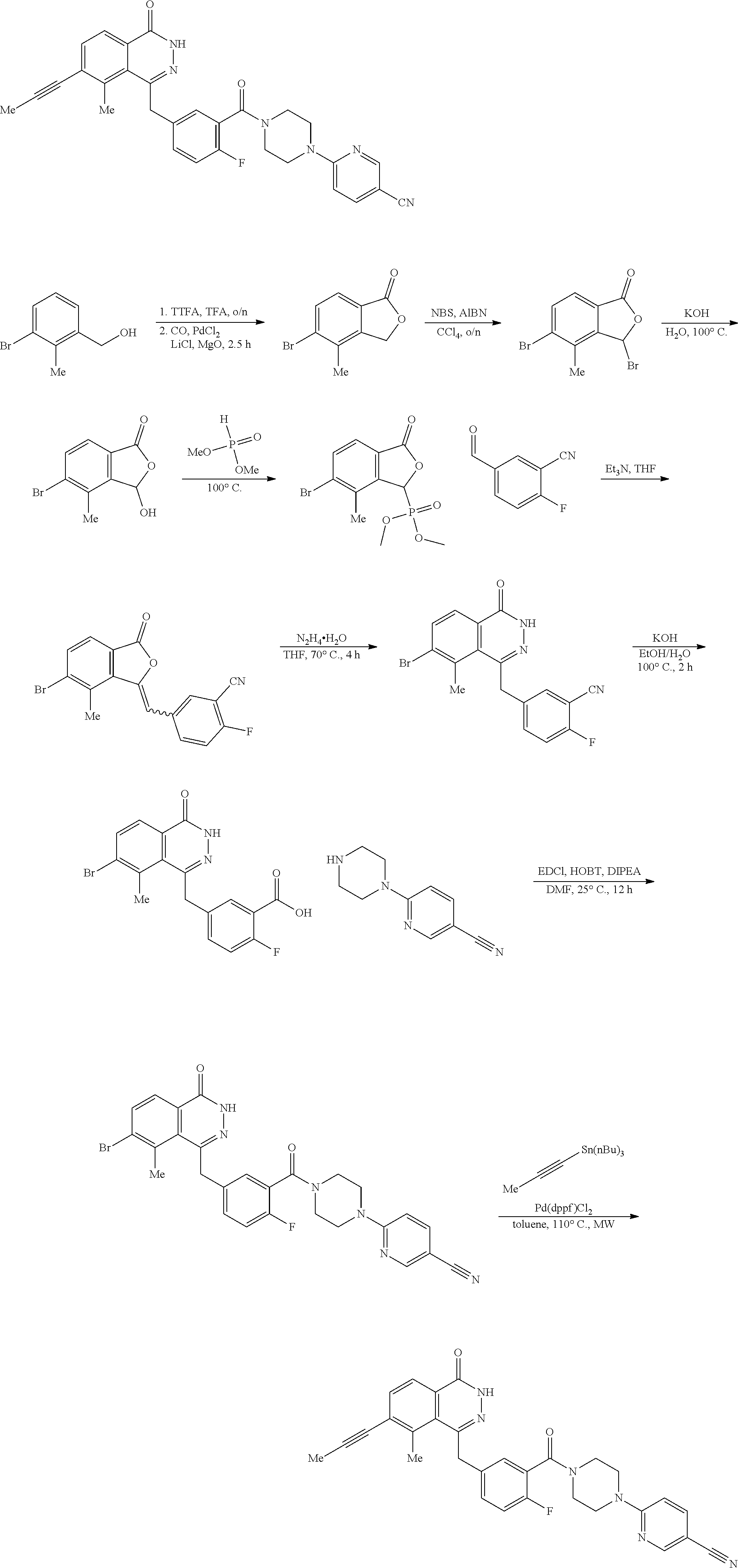

### Step 1

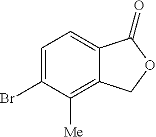 [SMILES: Cc1c(Br)ccc2c1COC2=O]

### 5-Bromo-4-methylisobenzofuran-1(3H)-one

Into a single neck round bottom flask were added (3-bromo-2-methyl-phenyl)methanol (2.7 g, 13.43 mmol), TFA (30 mL) and LiCl (1.14 g, 26.86 mmol, 550.53 μL) at room temperature. The mixture was stirred overnight. The reaction was concentrated under reduced pressure. To the residue were added MeOH (70 mL), MgO (1.11 g, 26.86 mmol), PdCl₂ (237.69 mg, 1.34 mmol) and TTFA (7.29 g, 13.43 mmol) at room temperature. The mixture was stirred for 2 hr at room temperature under CO atmosphere. The mixture was diluted in EA, washed with water. The organic phase was dried over Na₂SO₄, concentrated under reduced pressure. The residue was purified by silica gel chromatography to give 5-bromo-4-methyl-3H-isobenzofuran-1-one (2.4 g, 57% yield) as a white solid. ¹H NMR (400 MHz, DMSO-D6) δ 7.87 (d, J=8.1 Hz, 1H), 7.67-7.64 (m, 1H), 5.49 (s, 2H), 2.39 (s, 3H).

### Step 2

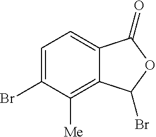 [SMILES: Cc1c(Br)ccc2c1C(Br)OC2=O]

### 3,5-Dibromo-4-methylisobenzofuran-1(3H)-one

Into a 100 ml single round bottom flask were added 5-bromo-4-methyl-3H-isobenzofuran-1-one (900 mg, 3.96 mmol) CCl₄ (12 mL), AIBN (65.01 mg, 396.38 μmol) and NBS (917.12 mg, 5.15 mmol, 437.14 μL) at room temperature. The mixture was stirred overnight at 80° C. under nitrogen atmosphere. The solvent was removed under reduced pressure. The residue was purified by silica gel chromatography (petroleum ether:EtOAc=15:1) to give 3,5-dibromo-4-methyl-3H-isobenzofuran-1-one (880 mg, 73% yield) as a white solid. ¹H NMR (400 MHz, CDCl₃) δ 7.83 (d, J=8.1 Hz, 1H), 7.64 (d, J=8.1 Hz, 1H), 7.30 (d, J=5.2 Hz, 1H), 2.48 (s, 3H).

### Step 3

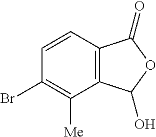 [SMILES: Cc1c(Br)ccc2c1C(O)OC2=O]

### 5-Bromo-3-hydroxy-4-methylisobenzofuran-1(3H)-one

Into a 100 mL single round bottom flask were added 3,5-dibromo-4-methyl-3H-isobenzofuran-1-one (850 mg, 2.78 mmol). H₂O (14 mL) and KOH (779.43 mg, 13.89 mmol, 381.33 μL) at room temperature. The mixture was stirred for 40 min at 100° C. under nitrogen atmosphere. The reaction was allowed to cool to room temperature. The solution was adjusted to PH 1.0 with 1 mol/L HCl aqueous solution. The precipitate was filtered. The filtrate was extracted with DCM. The organic phase was dried over Na₂SO₄. The precipitate was dissolved in the organic phase and concentrated under reduced pressure to give 5-bromo-3-hydroxy-4-methyl-3H-isobenzofuran-1-one (600 mg, 89% yield) as a white solid. LCMS ESI m/z 244.8 [M+H]⁺.

### Step 4

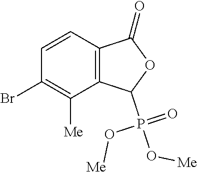 [SMILES: COP(=O)(OC)C1OC(=O)c2ccc(Br)c(C)c21]

### Dimethyl 6-bromo-7-methyl-3-oxo-1,3-dihydroisobenzofuran-1-ylphosphonate

Into a 100 mL single round bottom flask were added 5-bromo-3-hydroxy-4-methyl-3H-isobenzofuran-1-one (600) mg, 2.47 mmol) and dimethyl phosphonate (8 mL) at room temperature. The solution was stirred for 16 hr at 100° C. under nitrogen atmosphere. The temperature was allowed to cool to room temperature. EA and water were added to the solution. The organic phase was dried over Na₂SO₄, concentrated under reduced pressure. The residue was purified by silica gel chromatography (EtOAc:Petroleum ether=2:1) to give dimethyl 6-bromo-7-methyl-3-oxo-1,3-dihydroisobenzofuran-1-ylphosphonate (380 mg, 46% yield) as a white solid. LCMS ESI m/z 337.0 [M+H]⁺.

### Step 5

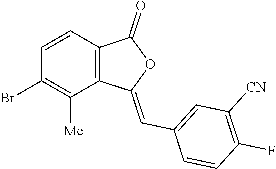 [SMILES: Cc1c(Br)ccc2c1/C(=C/c1ccc(F)c(C#N)c1)OC2=O]

### 5-((6-bromo-7-methyl-3-oxoisobenzofuran-1(3H)-ylidene)methyl)-2-fluorobenzonitrile

Into a 100 mL single round bottom flask were added dimethyl 6-bromo-7-methyl-3-oxo-1,3-dihydroisobenzofuran-1-ylphosphonate (300 mg, 895.29 μmol) THF (20 mL) 2-fluoro-5-formyl-benzonitrile (226.96 mg, 1.52 mmol) and Et₃N (271.78 mg, 2.69 mmol, 374.36 μL) at room temperature. The solution was stirred for 24 hr at room temperature under nitrogen atmosphere. The reaction was quenched with NaHSO₃ (aq) (30 mL), extracted with EA (30 mL), washed with water (20 mL). The organic phase was dried over Na₂SO₄, concentrated under reduced pressure. The residue was purified by triturated with DCM. The white solids were collected by filtration to give 5-((6-bromo-7-methyl-3-oxoisobenzofuran-1(3H)-ylidene)methyl)-2-fluorobenzonitrile (110 mg, 34% yield) as a white solid. ¹H NMR (400 MHz, DMSO-D6) δ 8.37 (dd, J=6.4, 2.2 Hz, 1H), 8.34-8.27 (m, 1H), 7.97 (d, J=8.1 Hz, 1H), 7.76 (d, J=8.0 Hz, 1H), 7.68 (t, J=9.1 Hz, 1H), 6.93 (s, 1H), 2.78 (s, 3H).

### Step 6

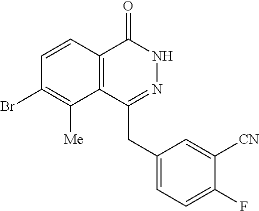 [SMILES: Cc1c(Br)ccc2c(=O)[nH]nc(Cc3ccc(F)c(C#N)c3)c12]

### 5-((7-bromo-8-methyl-4-oxo-3,4-dihydrophthalazin-1-yl)methyl)-2-fluorobenzonitrile

To a mixture of 5-((6-bromo-7-methyl-3-oxoisobenzofuran-1(3H)-ylidene)methyl)-2-fluorobenzonitrile (110 mg, 307.12 μmol) in THF (3 mL) was added hydrazine hydrate (38.44 mg, 614.25 μmol, 37.43 μL) at 25° C. After stirring the reaction mixture at 70° C. for 3 hr. Water and EtOAc were added to the mixture, which became two phases. The organic layer was separated, washed with NaCl (aq), dried over Na₂SO₄ and concentrated to give 5-((7-bromo-8-methyl-4-oxo-3,4-dihydrophthalazin-1-yl)methyl)-2-fluorobenzonitrile (110 mg, 96.2% yield) as a yellow solid. LCMS ESI m/z 372.0 [M+H]⁺.

### Step 7

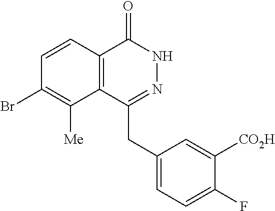 [SMILES: Cc1c(Br)ccc2c(=O)[nH]nc(Cc3ccc(F)c(C(=O)O)c3)c12]

### 5-((7-Bromo-8-methyl-4-oxo-3,4-dihydrophthalazin-1-yl)methyl)-2-fluorobenzoic acid

To a solution of 5-[(7-bromo-8-methyl-4-oxo-3H-phthalazin-1-yl)methyl]-2-fluoro-benzonitrile (110 mg, 295.55 μmol) in water (4 mL) and EtOH (1 mL) was added KOH (165.83 mg, 2.96 mmol, 81.13 μL) at 25° C. After stirring the reaction mixture at 100° C. for 2 hr. Ethanol was evaporated off and the aqueous was extracted with ethyl acetate. The aqueous was then acidified with conc HCl until the solution reached pH 1. The solids were collected by filtration and dried under vacuum to give 5-((7-bromo-8-methyl-4-oxo-3,4-dihydrophthalazin-1-yl)methyl)-2-fluorobenzoic acid (95 mg, crude) as a red solid. LCMS ESI m/z 390.7 [M+H]⁺.

### Step 8

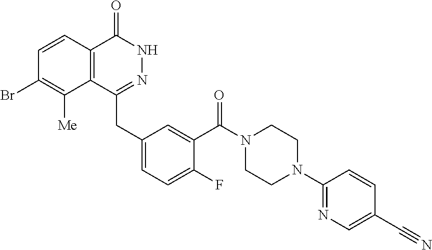 [SMILES: Cc1c(Br)ccc2c(=O)[nH]nc(Cc3ccc(F)c(C(=O)N4CCN(c5ccc(C#N)cn5)CC4)c3)c12]

### 6-(4-(5-((7-bromo-8-methyl-4-oxo-3,4-dihydrophthalazin-1-yl)methyl)-2-fluorobenzoyl)piperazin-1-yl)nicotinonitrile

Following the amide coupling procedure in Example 9, but starting with 5-((7-Bromo-8-methyl-4-oxo-3,4-dihydrophthalazin-1-yl)methyl)-2-fluorobenzoic acid and 6-(piperazin-1-yl)nicotinonitrile gave the title compound as a red solid. LCMS ESI m/z 560.7 [M+H]⁺.

### Step 9

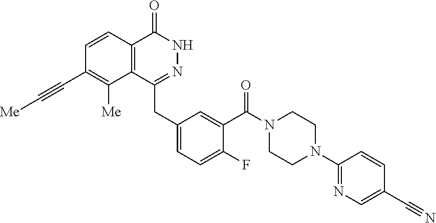 [SMILES: CC#Cc1ccc2c(=O)[nH]nc(Cc3ccc(F)c(C(=O)N4CCN(c5ccc(C#N)cn5)CC4)c3)c2c1C]

### 6-(4-(2-Fluoro-5-((8-methyl-4-oxo-7-(prop-1-ynyl)-3,4-dihydrophthalazin-1-yl)methyl)benzoyl)piperazin-1-yl)nicotinonitrile

To a solution of 6-[4-[5-[(7-bromo-8-methyl-4-oxo-3H-phthalazin-1-yl)methyl]-2-fluoro-benzoyl]piperazin-1-yl]pyridine-3-carbonitrile (110 mg, 195.94 μmol) in toluene (1 mL) was added tributyl(prop-1-ynyl)stannane (128.97 mg, 391.87 μmol), Pd(PPh₃)₄ (22.64 mg, 19.59 μmol) at 25° C. under N₂ (g). The reaction was stirred under microwave at 100° C. for 1 hr. The reaction mixture was filtered and concentrated. The residue was purified by prep-HPLC to give 6-[4-[2-fluoro-5-[(8-methyl-4-oxo-7-prop-1-ynyl-3H-phthalazin-1-yl)methyl]benzoyl]piperazin-1-yl]pyridine-3-carbonitrile (12.4 mg, 12% yield) as a white solid. LCMS ESI m/z 520.7 [M+H]⁺. ¹H NMR (400 MHz, DMSO-d₆) δ 12.65 (s, 1H), 8.51 (d, J=2.3 Hz, 1H), 8.13 (d, J=8.2 Hz, 1H), 7.90 (dd, J=9.1, 2.4 Hz, 1H), 7.78 (d, J=8.2 Hz, 1H), 7.29-7.19 (m, 3H), 6.92 (d, J=9.2 Hz, 1H), 4.51 (s, 2H), 3.73 (d, J=13.0 Hz, 4H), 3.61 (s, 2H), 3.34 (d, J=3.2 Hz, 2H), 2.79 (s, 3H), 2.14 (s, 3H).

### Synthesis of Example 87: 6-(4-(3-((5,6-Difluoro-4-oxo-3,4-dihydrophthalazin-1-yl)methyl)benzoyl)piperazin-1-yl)nicotinonitrile

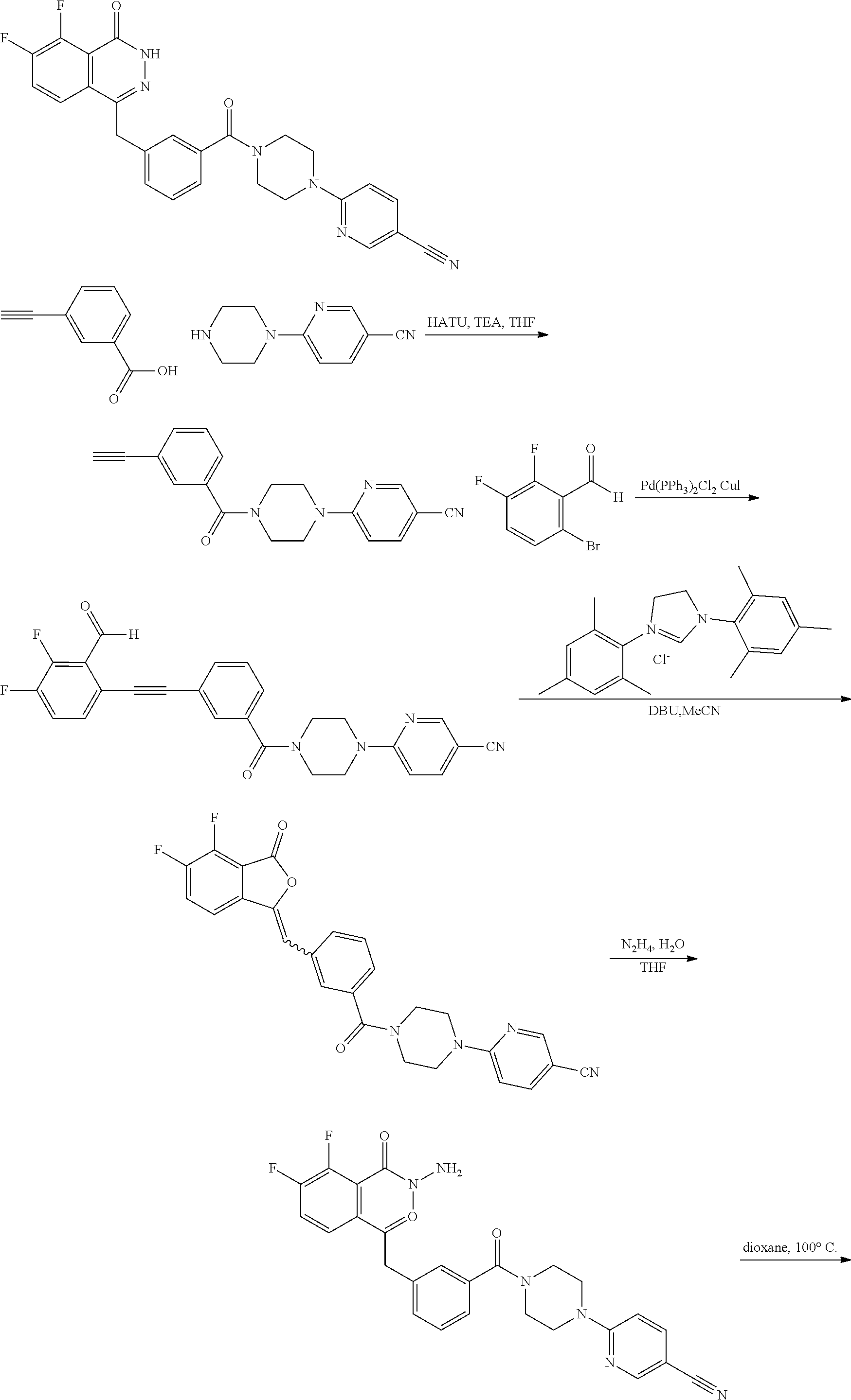

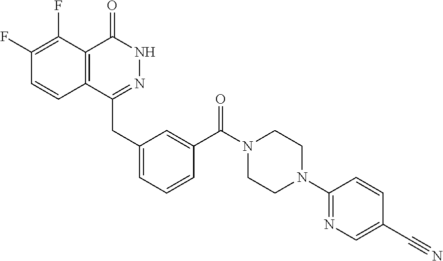 [SMILES: N#Cc1ccc(N2CCN(C(=O)c3cccc(Cc4n[nH]c(=O)c5c(F)c(F)ccc45)c3)CC2)nc1]

### Step 1

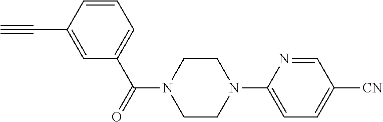 [SMILES: C#Cc1cccc(C(=O)N2CCN(c3ccc(C#N)cn3)CC2)c1]

### 6-(4-(3-Ethynylbenzoyl)piperazin-1-yl)nicotinonitrile

To a solution of 3-ethynylbenzoic acid (4.0 g, 21.3 mmol) in THF (30 mL) was added 6-(piperazin-1-yl)nicotinonitrile (3.1 g, 21.3 mmol), HATU (9.7 g, 25.5 mmol) and DIPEA (8.2 g, 63.8 mmol). The resulting mixture was stirred at 20° C. for 2 h. Then water (30 mL) and EtOAc (40 mL) were added to the mixture, which was became two phases. The organic layer was separated, washed with water (10 mL×2), aq. NH₄Cl (10 mL×2), brine (10 mL×2), dried over Na₂SO₄ and concentrated to give a residue. The residue was purified by silica gel chromatography (PE/EA=10/1 to 2/1, v/v) to give 6-(4-(3-ethynylbenzoyl)piperazin-1-yl)nicotinonitrile (6.0 g, 89% yield) as a white solid. LCMS ESI m/z 317.1 [M+H]⁺.

### Step 2

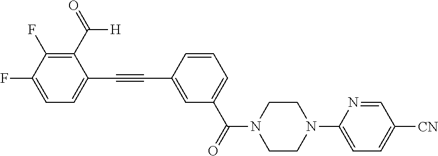 [SMILES: N#Cc1ccc(N2CCN(C(=O)c3cccc(C#Cc4ccc(F)c(F)c4C=O)c3)CC2)nc1]

### 6-(4-(3-((3,4-Difluoro-2-formylphenyl)ethynyl)benzoyl)piperazin-1-yl)nicotinonitrile

A mixture of 6-(4-(3-ethynylbenzoyl)piperazin-1-yl)nicotinonitrile (3 g, 9.5 mmol) in DMF (15 mL) was added 6-bromo-2,3-difluorobenzaldehyde (2.1 g, 9.5 mmol), Pd(PPh₃)₂Cl₂ (0.7 g, 1.0 mmol), CuI (0.2 g, 1.0 mmol) and ET3N (2.9 g, 28.5 mmol). The resulting mixture was stirred at rt for 1 h. The mixture was concentrated to give a residue. The residue was purified by triturated with petroleum ether/EtOAc (Petroleum ether/EtOAc=5/1) (20 mL) to give 6-(4-(3-((3,4-difluoro-2-formylphenyl)ethynyl)benzoyl)piperazin-1-yl)nicotinonitrile (2.0 g, 46% yield) as a yellow solid. LCMS ESI m/z 457.1 [M+H]⁺.

### Step 3

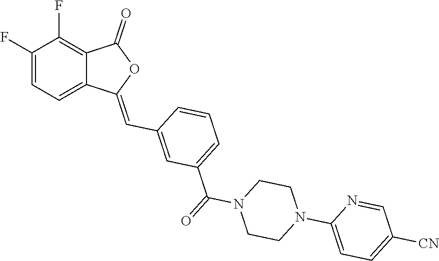 [SMILES: N#Cc1ccc(N2CCN(C(=O)c3cccc(/C=C4\OC(=O)c5c4ccc(F)c5F)c3)CC2)nc1]

### 6-(4-(3-((4,5-difluoro-3-oxoisobenzofuran-1(3H)-ylidene)methyl)benzoyl)piperazin-1-yl)nicotinonitrile

To a solution of 6-(4-(3-((3,4-difluoro-2-formylphenyl)ethynyl)benzoyl)piperazin-1-yl)nicotinonitrile (1.0 g, 2.2 mmol) in MeCN (10 mL) was added 1,3-dimesitylimidazolidin-1-ium chloride (113 mg, 0.3 mmol) and DBU (100 mg, 0.7 mmol). The resulting mixture was stirred at 80′C for 2 h under O₂. Water (10 mL) and EtOAc (20 mL) were added to the mixture, which became two phases. The organic layer was separated, washed with brine (10 mL×2), dried over Na₂SO₄ and concentrated to a residue. The residue was purified by silica gel chromatography (PE/EA=10/1 to 2/1, v/v) to give (Z)-6-(4-(3-((4,5-difluoro-3-oxoisobenzofuran-1(3H)-ylidene)methyl)benzoyl)piperazin-1-yl)nicotinonitrile (500 mg, 48% yield) as a yellow solid. LCMS ESI m/z 473.1 [M+H]⁺.

### Step 4

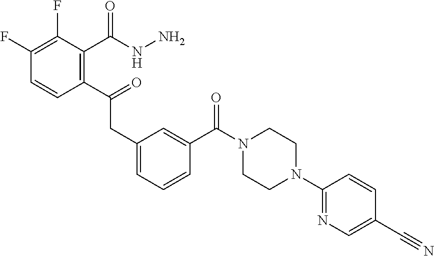 [SMILES: N#Cc1ccc(N2CCN(C(=O)c3cccc(CC(=O)c4ccc(F)c(F)c4C(=O)NN)c3)CC2)nc1]

### 6-(2-(3-(4-(5-Cyanopyridin-2-yl)piperazine-1-carbonyl)phenyl)acetyl)-2,3-difluorobenzohydrazide

Following the procedure of step 5 in Example 6, but starting with 6-(4-(3-((4,5-difluoro-3-oxoisobenzofuran-1(3H)-ylidene)methyl)benzoyl)piperazin-1-yl)nicotinonitrile gave the title compound as a yellow solid. LCMS ESI m/z 504.1 [M+H]⁺.

### Step 5

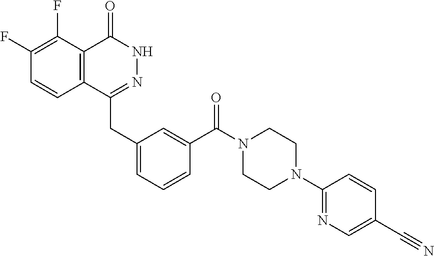 [SMILES: N#Cc1ccc(N2CCN(C(=O)c3cccc(Cc4n[nH]c(=O)c5c(F)c(F)ccc45)c3)CC2)nc1]

### 6-(4-(3-((5,6-difluoro-4-oxo-3,4-dihydrophthalazin-1-yl)methyl)benzoyl)piperazin-1-yl)nicotinonitrile

Following the procedure of step 6 in Example 6 but starting with 6-(2-(3-(4-(5-cyanopyridin-2-yl)piperazine-1-carbonyl)phenyl)acetyl)-2,3-difluorobenzohydrazide gave the title compound as a yellow solid. LCMS ESI m/z 486.8 [M+H]⁺. ¹H NMR (400 MHz, DMSO-d₆) δ 12.64 (s, 1H), 8.51 (m, 1H), 8.03 (m, 1H), 7.88 (m, 1H), 7.82 (m, 1H), 7.41 (m, 3H), 7.28 (m, 1H), 6.91 (m, 1H), 4.34 (s, 2H), 3.68 (m, 6H), 3.36 (m, 2H).

### Synthesis of Example 88: 6-(4-(3-((5-Fluoro-4-oxo-3,4-dihydrophthalazin-1-yl)methyl)benzoyl)piperazin-1-yl)nicotinonitrile

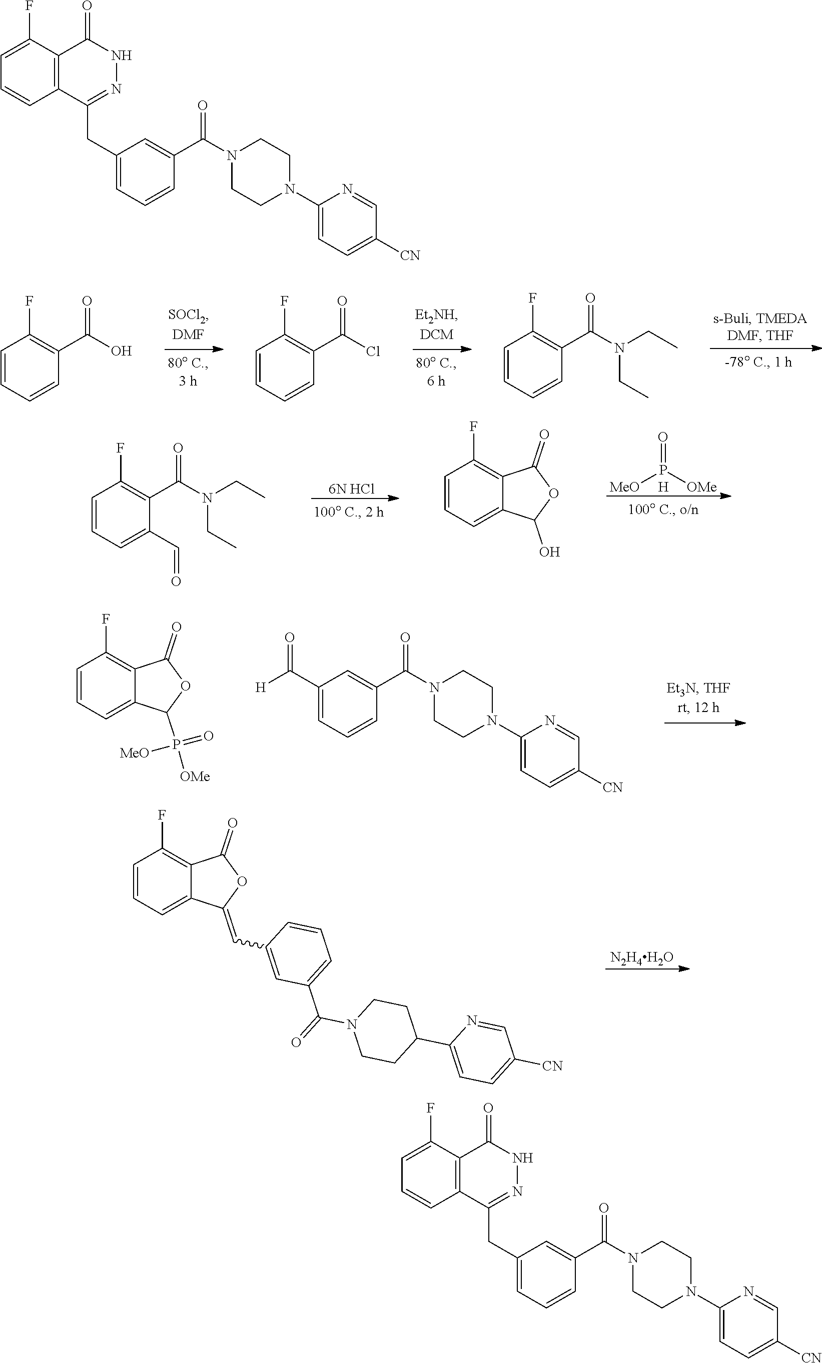

### Step 1

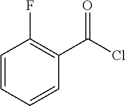

### 2-Fluorobenzoyl chloride

2-Fluorobenzoic acid (20.0 g, 142.74 mmol) was added into sulfurous dichloride (15 mL) slowly at 0° C. Then NN-dimethylformamide (1 mL) was added dropwise to the solution at 0° C. The mixture was stirred at 80° C. for 3 hr. The reaction mixture was concentrated under reduced pressure to give 2-fluorobenzoyl chloride (22.6 g, 100% yield) as brown oil.

### Step 2

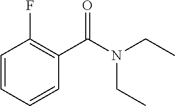

### N,N-Diethyl-2-fluorobenzamide

To a solution of 2-fluorobenzoyl chloride (22.6 g, 142.54 mmol) in dichloromethane (100 mL) was added diethylamine (22.1 mL, 213.80 mmol). The resulting mixture was stirred at 80° C. for 6 hr. The mixture was quenched with water (200 mL), extracted with dichloromethane (200 mL×2). The combined organic phase was washed successively with hydrochloric acid (200 mL×2, 1 M) and saturated sodium chloride solution (200 mL), dried over sodium sulfate, filtered and concentrated under reduced pressure to give N,N-diethyl-2-fluorobenzamide (27.8 g, 100% yield) as a brown oil.

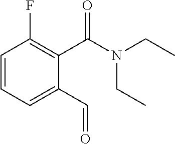 [SMILES: CCN(CC)C(=O)c1c(F)cccc1C=O]

### N,N-Diethyl-2-fluoro-6-formylbenzamide

To a solution of N,N-diethyl-2-fluorobenzamide (10.0 g, 51.22 mmol) in tetrahydrofuran (100 mL) was added N,N,N′,N′-Tetramethylethylenediamine (TMEDA, 16.9 mL, 112.68 mmol). sec-Butyllithium (s-BuLi, 88.7 mL, 112.68 mmol, 1.3 M in hexane) was added dropwise at −78° C. under nitrogen atmosphere and then the mixture was stirred at −78° C. for 0.5 hr under nitrogen atmosphere. N,N-dimethylformamide (19.8 mL, 256.10 mmol) was added slowly at −78° C. under nitrogen atmosphere. The resulting mixture was stirred at −78° C. for 1 h under nitrogen atmosphere. The mixture was quenched with hydrochloric acid (200 mL, 1 M), extracted with ethyl acetate (200 mL×2). The combined organic phase was washed saturated sodium chloride solution (200 mL), dried over sodium sulfate, filtered and concentrated under reduced pressure. The residue was purified by silica gel chromatography (petroleum ether/ethyl acetate=10/1, v/v) to give NN-diethyl-2-fluoro-6-formylbenzamide (4.75 g, 42% yield) as a yellow oil. LCMS ESI m/z: 223.9 [M+H]⁺.

### Step 4

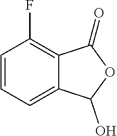 [SMILES: O=C1OC(O)c2cccc(F)c21]

### 7-Fluoro-3-hydroxyisobenzofuran-1(3H)-one

A mixture of N,N-diethyl-2-fluoro-6-formylbenzamide (4.75 g, 21.28 mmol) was stirred at 100° C. for 2 hr. The mixture was diluted with water (100 mL), extracted with ethyl acetate (100 mL×2). The combined organic phase was washed with saturated sodium chloride solution (100 mL), dried over sodium sulfate, filtered and concentrated under reduced pressure. The residue was purified by silica gel chromatography (petroleum ether/ethyl acetate=4/1, v/v) to give 7-fluoro-3-hydroxyisobenzofuran-1(3H)-one (2.19 g, 61% yield) as a white solid.

### Step 5, 6 and 7

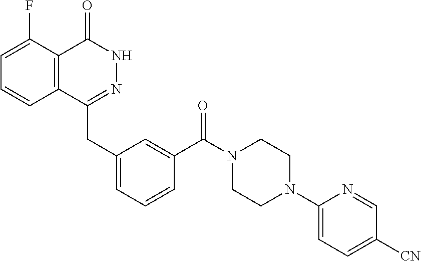 [SMILES: N#Cc1ccc(N2CCN(C(=O)c3cccc(Cc4n[nH]c(=O)c5c(F)cccc45)c3)CC2)nc1]

### 6-(4-(3-((5-Fluor-4-oxo-3,4-dihydrophthalazin-1-yl)methyl)benzoyl)piperazin-1-yl)nicotinonitrile

Following the three-step phthalazinone synthesis procedure in Example 1, but starting with 7-fluoro-3-hydroxyisobenzofuran-1(3H)-one gave the title compound as a yellow solid. LCMS EST m/z 468.8 [M+H]⁺. ¹H NMR (400 MHz, DMSO-d₆) δ 12.56 (s, 1H), 8.51 (d, J=4.0 Hz, 1H), 7.91-7.88 (m, 2H), 7.77 (d, J=8.0 Hz, 1H), 7.62-7.57 (m, 2H), 7.43-7.38 (m, 3H), 7.29 (d, J=8.0 Hz, 1H), 6.92 (d, J=8.0 Hz, 1H), 4.34 (s, 2H), 3.74-3.67 (m, 6H), 3.34-3.31 (m, 2H).

### Synthesis of Example 89: 6-(4-(2-Fluoro-5-((8-methoxy-4-oxo-7-phenoxy-3,4-dihydrophthalazin-1-yl)methyl)benzoyl)piperazin-1-yl)nicotinonitrile

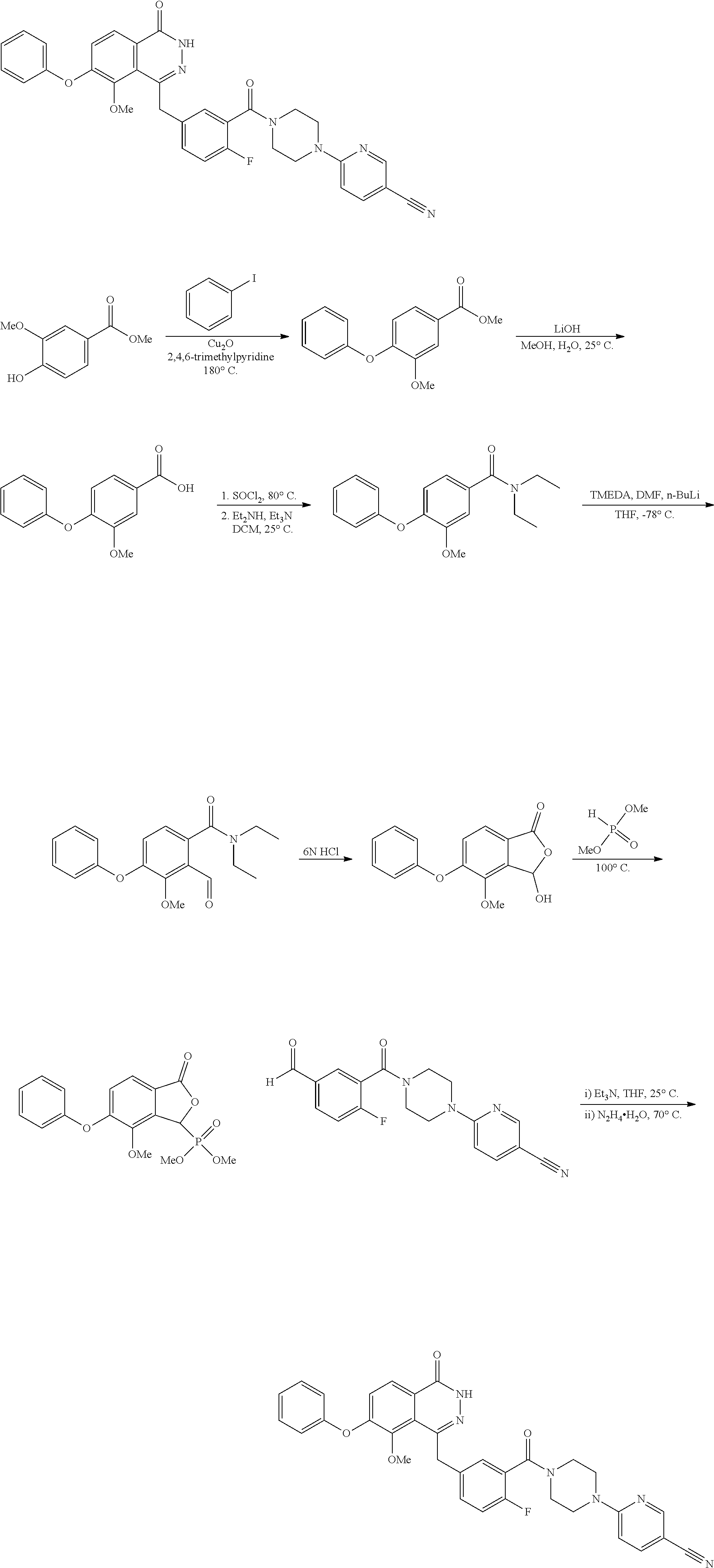

### Step 1

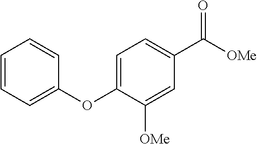 [SMILES: COC(=O)c1ccc(Oc2ccccc2)c(OC)c1]

### methyl 3-methoxy-4-phenoxybenzoate

A mixture of methyl 4-hydroxy-3-methoxybenzoate (5 g, 27.45 mmol), iodobenzene (11.20 g, 0.30 mmol) and CuO (9.43 g, 65.87 mmol) in 2,4,6-trimethylpyridine (10 mL) was stirred at 180° C. for 2 hours. The mixture was cooled to rt, diluted with H₂O (10 mL) and extracted with EtOAc (20 mL×3). The combined organic layer was washed with brine, dried over anhydrous Na₂SO₄, and filtered and concentrated in vacuo. The residue was purified by prep-HPLC to give methyl 3-methoxy-4-phenoxybenzoate (4.90 g, 69% yield) as a white solid. LCMS ESI m/z: 259 [M+H]⁺.

### Step 2

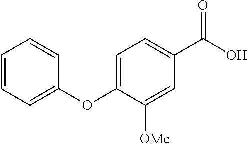 [SMILES: COc1cc(C(=O)O)ccc1Oc1ccccc1]

### 3-methoxy-4-phenoxybenzoic acid

A solution of methyl 3-methoxy-4-phenoxybenzoate (4.90 g, 18.97 mmol) and LiOH (3.41 g, 142.27 mmol) in MeOH (30 mL) and H₂O (10 mL) was stirred at rt for 1 hour. The organic solvent was removed in vacuum and aq HCl (1N) was added until the solution reached to pH 5˜6. The solids were collected by filtration, washed with water and dried under vacuum to give 3-methoxy-4-phenoxybenzoic acid (4.54 mg, 97% yield) as a white solid. LCMS ESI m/z 245 [M+H]⁺. This crude product was used in the next step without further purification.

### Step 3

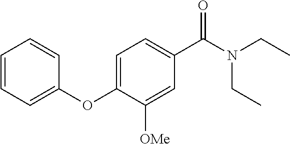 [SMILES: CCN(CC)C(=O)c1ccc(Oc2ccccc2)c(OC)c1]

### N,N-diethyl-3-methoxy-4-phenoxybenzamide

A mixture of 3-methoxy-4-phenoxybenzoic acid (4.24 g, 17.36 mmol) and thionyl chloride (41.31 g, 347.20 mmol) was stirred at 80° C. under Ar (g) for 3 hours. The reaction was concentrated under vacuum, the residue was dissolved in DCM (30 mL), then added a solution of Et₂NH (1.90 g, 26.04 mmol) and Et₃N (5.27 g, 52.08 mmol) in DCM (100 mL). The reaction was stirred at rt for 1 hour, then diluted with water (100 mL) and extracted with DCM (100 mL×3). The combined organic layer was dried over Na₂SO₄, filtered and concentrated in vacuo. The residue was purified by silica gel chromatography (EtOAc/petroleum ether=20 to 40%) to give N,N-diethyl-3-methoxy-4-phenoxybenzamide (5.28 g, 95% yield) as a yellow oil. LCMS ESI m/z 300 [M+H]⁺.

### Step 4

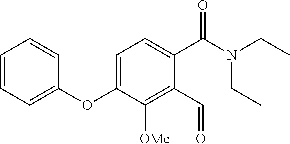 [SMILES: CCN(CC)C(=O)c1ccc(Oc2ccccc2)c(OC)c1C=O]

### N,N-diethyl-2-formyl-3-methoxy-4-phenoxybenzamide

A solution of N,N-diethyl-3-methoxy-4-phenoxybenzamide (600 mg, 2.00 mmol) and TMEDA (175 mg, 1.50 mmol) in THF (10 mL) was cooled to −78° C., then n-BuLi (2.5M in heptane, 0.60 mL, 1.50 mmol) was added dropwise under Argon. The reaction was stirred at −78° C. for 30 min, then added DMF (123 mg, 1.70 mmol) dropwise. The reaction was stirred at −78° C. for 20 min, then quenched with sat. NH₄Cl solution (10 mL) and neutralized aq HCl (1N) until the solution reached pH 5˜6. The mixture was extracted with EtOAc (50×3 mL). The combined organic layer was washed with bine, dried over Na₂SO₄, filtered and concentrated in vacuo. The residue was purified by silica gel chromatography (EtOAc/pet ether=10% to 20%) to give N,N-diethyl-2-formyl-3-methoxy-4-phenoxybenzamide (160 mg, 27% yield) as a white solid. LCMS ESI m/z: 328 [M+H]⁺.

### Step 5

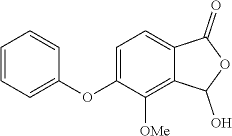 [SMILES: COc1c(Oc2ccccc2)ccc2c1C(O)OC2=O]

### 3-Hydroxy-4-methoxy-5-phenoxyisobenzofuran-1(3H)-one

A mixture of N,N-diethyl-2-formyl-3-methoxy-4-phenoxybenzamide (160 mg, 0.49 mmol) in 6 M HCl (5 mL) was stirred at 100° C. for 16 hours. The reaction was cooled to rt. The solid was collected by filtration, washed with water and dried under vacuum to give 3-hydroxy-4-methoxy-5-phenoxyisobenzofuran-1(3H)-one (150 mg, 95% yield) as a yellow solid. LCMS ESI m/z: 273 [M+H]⁺. This crude product was used in the next step without further purification.

### Step 6

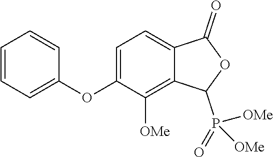 [SMILES: COc1c(Oc2ccccc2)ccc2c1C(P(=O)(OC)OC)OC2=O]

### 7-Methoxy-3-oxo-6-phenoxy-1,3-dihydroisobenzofuran-1-yl dimethyl phosphate

A solution of 3-hydroxy-4-methoxy-5-phenoxyisobenzofuran-1(3H)-one (150 mg, 0.55 mmol) in dimethyl phosphonate (1.21 g, 11.02 mmol) was stirred at 100° C. for 2 hours. The mixture was cooled to rt, diluted with H₂O (100 mL) and extracted with EtOAc (50 mL×3). The combined organic layer was washed with brine, dried over anhydrous Na₂SO₄, filtered and concentrated in vacuo. The residue was purified by silica gel chromatography (EtOAc/pet ether=10% to 15%) to afford 7-methoxy-3-oxo-6-phenoxy-1,3-dihydroisobenzofuran-1-yl dimethyl phosphate (95 mg, 20% yield) as a white solid. LCMS ESI m/z: 381 [M+H]⁺.

### Step 7

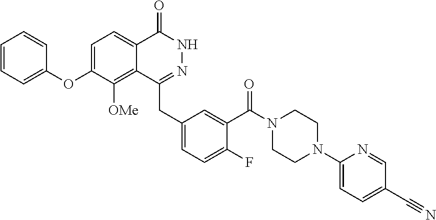 [SMILES: COc1c(Oc2ccccc2)ccc2c(=O)[nH]nc(Cc3ccc(F)c(C(=O)N4CCN(c5ccc(C#N)cn5)CC4)c3)c12]

### 6-(4-(2-Fluoro-5-((8-methoxy-4-oxo-7-phenoxy-3,4-dihydrophthalazin-1-yl)methyl) benzoyl)piperazin-1-yl)nicotinonitrile

A solution of 7-methoxy-3-oxo-6-phenoxy-1,3-dihydroisobenzofuran-1-yl dimethyl phosphate (95 mg, 0.25 mmol), 6-(4-(2-fluoro-5-formylbenzoyl)piperazin-1-yl)nicotinonitrile (97.87 mg, 0.29 mmol) and Et₃N (80 mg, 0.79 mmol) in anhydrous THF (5 mL) was stirred at 25° C. under Ar (g) for 3 hours. Hydrazine hydrate (20 mg, 0.39 mmol) was added and the reaction was stirred at 70° C. for 2 hours. The reaction was cooled to rt, then THF was removed under vacuum. The formed solids were washed with water (10 mL) and pet ether/EtOAc (10:1, 10 mL) to give 6-(4-(2-fluoro-5-((8-methoxy-4-oxo-7-phenoxy-3,4-dihydrophthalazin-1-yl)methyl)benzoyl)piperazin-1-yl)nicotinonitrile (19 mg, 32% yield) as a yellow. LCMS ESI m/z: 591 [M+H]⁺. ¹H NMR (400 MHz, DMSO-D6) δ 12.63 (s, 1H), 8.50 (d, J=2.2 Hz, 1H), 8.05 (d, J=8.7 Hz, 1H), 7.89 (dd, J=9.1, 2.3 Hz, 1H), 7.46-7.35 (m, 3H), 7.34-7.28 (m, 1H), 7.22 (dt, J=14.8, 6.2 Hz, 3H), 7.05 (d, J=7.8 Hz, 2H), 6.90 (d, J=9.2 Hz, 1H), 4.39 (s, 2H), 3.79 (s, 3H), 3.78-3.68 (m, 4H), 3.64-3.56 (m, 2H), 3.31-3.25 (m, 2H).

### Synthesis of Example 90: 6-(4-(2-fluoro-5-((4-oxo-7-(m-tolyloxy)-3,4-dihydrophthalazin-1-yl)methyl)benzoyl)piperazin-1-yl)nicotinonitrile

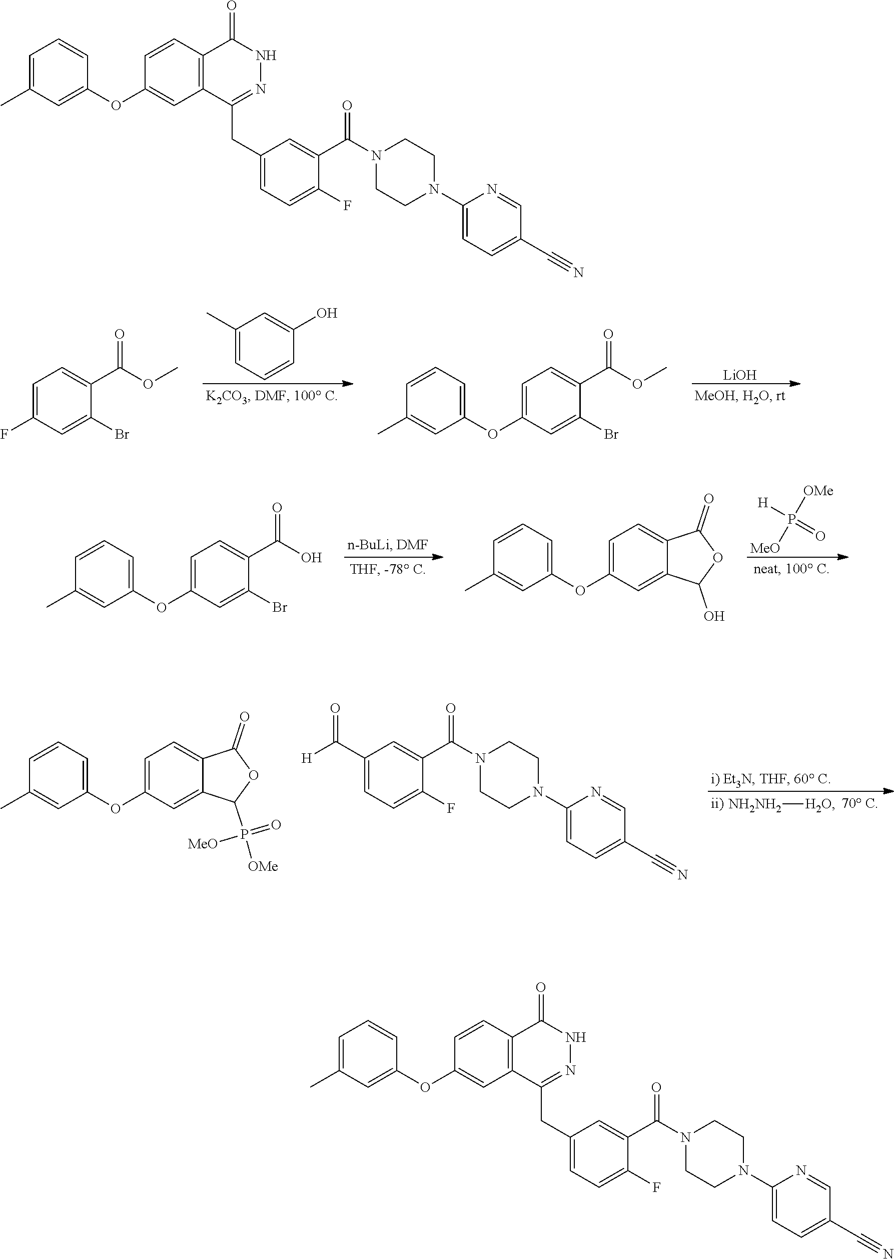

### Step 1

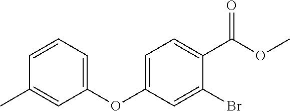

### Methyl 2-bromo-4-(m-tolyloxy)benzoate

A mixture of methyl methyl 2-bromo-4-fluorobenzoate (2.00 g, 8.58 mmol), K₂CO₃ (2.37 g, 17.16 mmol) and m-cresol (1.39 g, 12.87 mmol) in DMF (20 mL) was stirred at 100° C. for 16 hours. The reaction was cooled to rt, diluted with H₂O (100 mL) and extracted with EtOAc (100 mL×3). The combined organic layer was washed with brine, dried over anhydrous Na₂SO₄, filtered and concentrated in vacuum. The residue was purified by silica gel chromatography (EtOAc/petroleum ether=0% to 7%) to give methyl 2-bromo-4-(m-tolyloxy)benzoate (2.45 g, 89% yield) as a colorless oil. LCMS ESI m/z 321.0 [M+H]⁺.

### Step 2

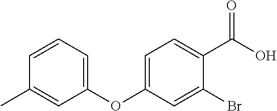 [SMILES: Cc1cccc(Oc2ccc(C(=O)O)c(Br)c2)c1]

### 2-Bromo-4-(m-tolyloxy)benzoic acid

A mixture of methyl methyl 2-bromo-4-(m-tolyloxy)benzoate (2.45 g, 7.63 mmol) and LiOH (1.83 g, 76.28 mmol) in MeOH (21 mL) and H₂O (7 mL) was stirred at rt for 2 hours. The organic solvent was removed in vacuo and aq HCl (1N) was added until the solution reached pH 5˜6. The formed solid was collected by filtration and dried under vacuum to give 2-bromo-4-(m-tolyloxy)benzoic (2.3 g, 98% yield) as a white solid. LCMS ESI m/z 307.0 [M+H]⁺.

### Step 3

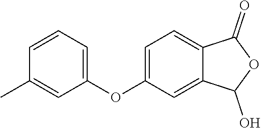 [SMILES: Cc1cccc(Oc2ccc3c(c2)C(O)OC3=O)c1]

### 3-Hydroxy-5-(m-tolyloxy)isobenzofuran-1(3H)-one

A solution of 2-bromo-4-(m-tolyloxy)benzoic acid (2.0 g, 6.51 mmol) in THF (35 mL) was cooled to −78° C. then n-BuLi (2.5M in heptane, 5.21 mL, 13.02 mmol) was added dropwise under Ar (g). The reaction was stirred at −78° C. for 30 min, then added DMF (1.05 g, 14.33 mmol). The reaction was stirred at −78° C. for 20 min, then quenched with sat. NH₄Cl solution (10 mL), neutralized by aq HCl (1N) until the solution reached pH 5˜6 and extracted with EtOAc (30×3 mL). The combined organic layer was washed with bine, dried over Na₂SO₄ and filtered. The residue was purified by silica gel chromatography (EtOAc/petroleum ether=30% to 50%) to give 3-hydroxy-5-(m-tolyloxy)isobenzofuran-1(3H)-one (900 mg, 54% yield) as a white solid. LCMS ESI m/z 257.1 [M+H]⁺.

### Step 4

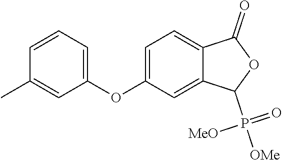 [SMILES: COP(=O)(OC)C1OC(=O)c2ccc(Oc3cccc(C)c3)cc21]

### Dimethyl (3-oxo-6-(m-tolyloxy)-1,3-dihydroisobenzofuran-1-yl)phosphonate

A solution of 3-hydroxy-5-(m-tolyloxy)isobenzofuran-1(3H)-one (500 mg, 1.95 mmol) in dimethyl phosphonate (5 mL) was stirred at 100° C. for 16 hours. The mixture was cooled to rt, diluted with H₂O (10 mL) and extracted with EtOAc (10 mL×3). The combined organic layer was washed with brine, dried over anhydrous Na₂SO₄, filtered and concentrated in vacuo. The residue was purified by silica gel chromatography (EtOAc/petroleum ether=50% to 60%) to give dimethyl (3-oxo-6-(m-tolyloxy)-1,3-dihydroisobenzofuran-1-yl)phosphonate (350 mg, 52% yield) as a off-white solid. LCMS ESI m/z 349.1 [M+H]⁺.

### Step 5

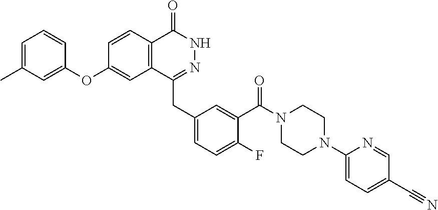 [SMILES: Cc1cccc(Oc2ccc3c(=O)[nH]nc(Cc4ccc(F)c(C(=O)N5CCN(c6ccc(C#N)cn6)CC5)c4)c3c2)c1]

### 6-(4-(2-Fluoro-5-((4-oxo-7-(m-tolyloxy)-3,4-dihydrophthalazin-1-yl)methyl)benzoyl)piperazin-1-yl)nicotinonitrile

A solution of dimethyl (3-oxo-6-(m-tolyloxy)-1,3-dihydroisobenzofuran-1-yl)phosphonate (250 mg, 0.72 mmol), 6-(4-(2-fluoro-5-formylbenzoyl)piperazin-1-yl)nicotinonitrile (255 mg, 0.75 mmol) and Et₃N (218 mg, 2.15 mmol) in anhydrous THF (5 mL) was stirred at 60° C. under Ar (g) for 15 hours. Hydrazine hydrate (55 mg, 0.93 mmol) was added and the reaction was stirred at 70° C. for 1 hour. The reaction was cooled to rt, then THF was removed under vacuum. The formed solid was washed with water (10 mL) and petroleum ether/EtOAc (2:1, 10 mL) to give 6-(4-(2-fluoro-5-((4-oxo-7-(m-tolyloxy)-3,4-dihydrophthalazin-1-yl)methyl)benzoyl)piperazin-1-yl)nicotinonitrile (343 mg, 83% yield) as a white solid. LCMS ESI m/z 575.1 [M+H]⁺. ¹H NMR (400 MHz, DMSO-d₆) δ 12.55 (s, 1H), 8.49 (d, J=2.2 Hz, 1H), 8.24 (d, J=8.7 Hz, 1H), 7.87 (dd, =9.1, 2.3 Hz, 1H), 7.38-7.30 (m, 5H), 7.21 (t, J=9.3 Hz, 1H), 7.09 (d, J=7.5 Hz, 1H), 6.93-6.86 (m, 3H), 4.23 (s, 2H), 3.79-3.69 (m, 4H), 3.59 (s, 2H), 3.28 (s, 2H), 2.30 (s, 3H).

### Synthesis of Example 91: 6-(4-(2-fluoro-5-((7-methoxy-4-oxo-3,4-dihydropyrido[4,3-d]pyridazin-1-yl)methyl)benzoyl)piperazin-1-yl)nicotinonitrile

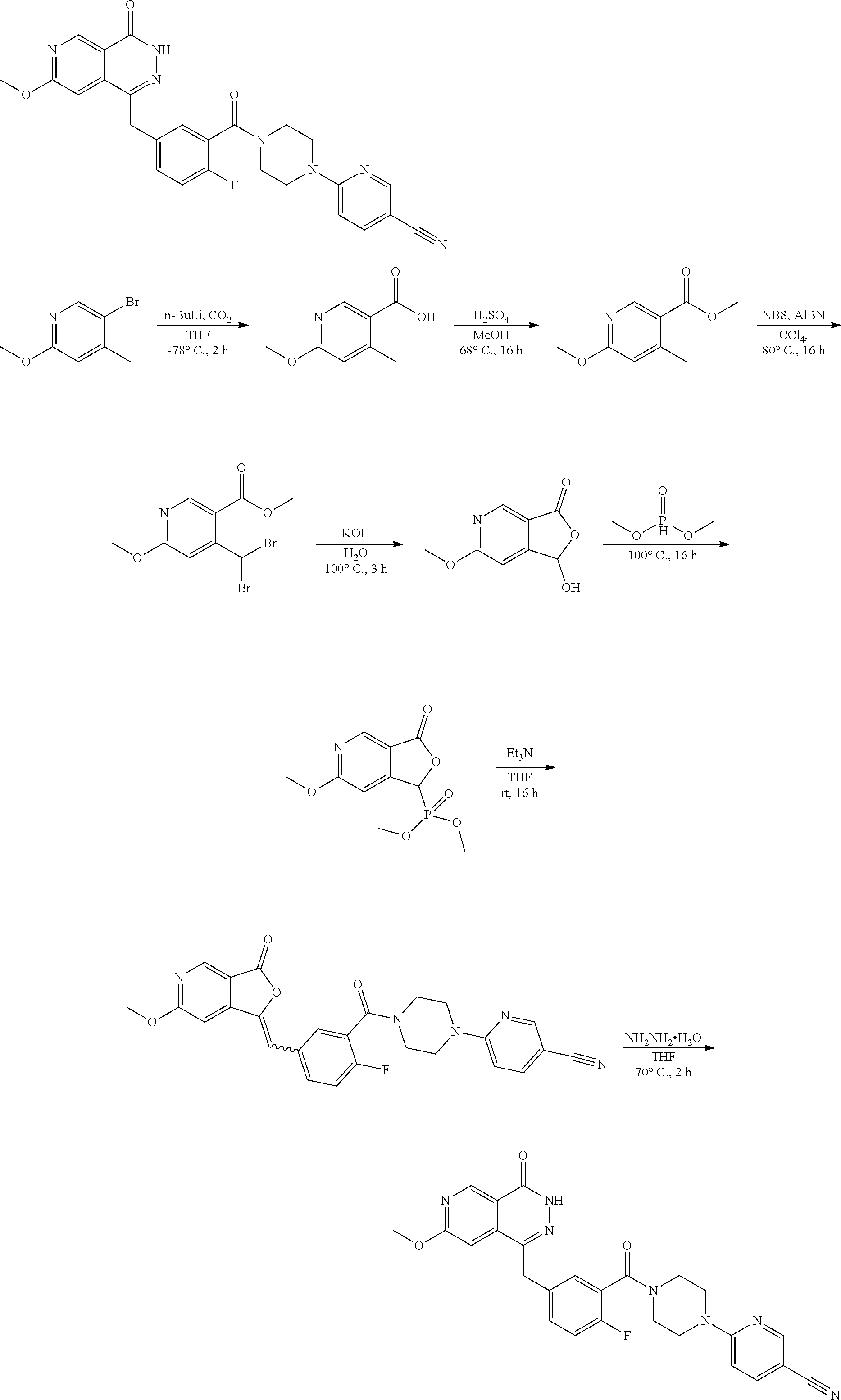

### Step 1

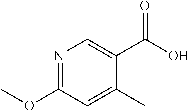

### 6-Methoxy-4-methylnicotinic acid

To a solution of 5-bromo-2-methoxy-4-methyl-pyridine (10 g, 49.49 mmol) in THF (100 mL) was dropwised with n-BuLi (2.5 M, 23.76 mL) under −78° C., and then stirred at −78° C. for 0.5 hr. The reaction mixture was charged with CO₂ balloon, and stirred at −78° C. for 2 hr, then 1 N HCl aq, was added to quench the reaction, warmed to rt. The solution was extracted with EA, washed with brine, dried and concentrated to give 6-methoxy-4-methyl-pyridine-3-carboxylic acid (15 g, crude) as a white solid.

### Step 2

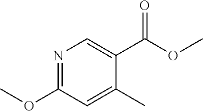

### Methyl 6-methoxy-4-methylnicotinate

To a solution of 6-methoxy-4-methyl-pyridine-3-carboxylic acid (15 g, 89.73 mmol) in MeOH (100 mL) was dropwised with H₂SO₄ (10 mL), and then heated to 68° C. for 16 hr. The reaction mixture was concentrated to give a residue, 100 mL H₂O was added, and then the solution was basitified until the solution reached pH 9 by sat. NaHCO₃, extracted with EtOAc (100 mL×3), washed with brine, dried and concentrated to give methyl 6-methoxy-4-methylnicotinate (1.3 g, 8.0% yield) as a white solid. ¹H NMR (400 MHz, CDCl₃) δ 8.75 (s, 1H), 6.58 (s, 1H), 3.97 (s, 3H), 3.88 (s, 3H), 2.57 (s, 3H).

### Step 3

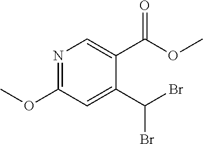

### Methyl 4-(dibromomethyl)-6-methoxynicotinate

To a sealed tube was added methyl 6-methoxy-4-methyl-pyridine-3-carboxylate (600 mg, 3.31 mmol), AIBN (10 mg), NBS (1.30 g, 7.29 mmol) and CCl₄ (18 mL), and then heated to 80° C. for 16 h. The mixture was concentrated and purified by silica gel chromatography to give methyl 4-(dibromomethyl)-6-methoxy-pyridine-3-carboxylate (950 mg, 85% yield) as a light yellow solid. ¹H NMR (400 MHz, CDCl₃) δ 8.78 (s, 1H), 7.95 (s, 1H), 7.43 (s, 1H), 4.03 (s, 3H), 3.94 (s, 3H).

### Step 4

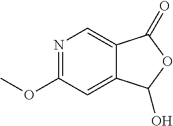

### 1-Hydroxy-6-methoxyfuro[3,4-c]pyridin-3(1H)-one

To a 25 mL round bottle was added methyl 4-(dibromomethyl)-6-methoxy-pyridine-3-carboxylate (350 mg, 1.03 mmol), KOH (289.67 mg, 5.16 mmol, 141.72 μL), H₂O (10 mL), and then heated to 100° C. for 3 hr. The mixture was acidified with 1 N HCl until the solution reached pH 5 and then extracted with EtOAc (15 mL×3), washed with brine, dried and concentrated to give 1-hydroxy-6-methoxy-1H-furo[3,4-c]pyridin-3-one (80 mg, 42.77% yield) as a light yellow solid.

### Step 5, 6 and 7

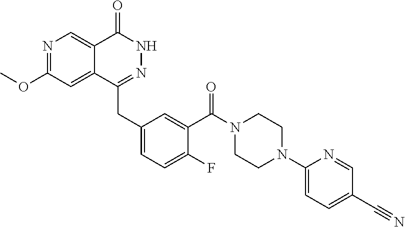 [SMILES: COc1cc2c(Cc3ccc(F)c(C(=O)N4CCN(c5ccc(C#N)cn5)CC4)c3)n[nH]c(=O)c2cn1]

### 6-(4-(2-Fluoro-5-((7-methoxy-4-oxo-3,4-dihydropyrido[4,3-d]pyridazin-1-yl)methyl)benzoyl)piperazin-1-yl)nicotinonitrile

Following the three-step phthalazinone synthesis procedure above in Example 1, but starting with 1-hydroxy-6-methoxyfuro[3,4-c]pyridin-3(1H)-one gave the title compound as a white solid. LCMS ESI m/z: 499.7 [M+H]⁺. ¹H NMR (400 MHz, DMSO-d6) δ 12.59 (s, 1H), 9.14 (s, 1H), 8.52 (d, J=2.1 Hz, 1H), 7.91 (dd, J=9.1, 2.1 Hz, 1H), 7.45 (t, J=6.8 Hz, 2H), 7.26 (t, J=8.9 Hz, 1H), 7.18 (s, 1H), 6.93 (d, J=9.2 Hz, 1H), 4.27 (s, 2H), 4.00 (s, 3H), 3.75 (d, J=12.2 Hz, 4H), 3.64 (s, 2H), 3.37 (s, 2H).

### Synthesis of Example 92: 6-(4-(2-Fluoro-5-((5-oxo-5,6-dihydropyrido[2,3-d]pyridazin-8-yl)methyl)benzoyl)piperazin-1-yl)nicotinonitrile

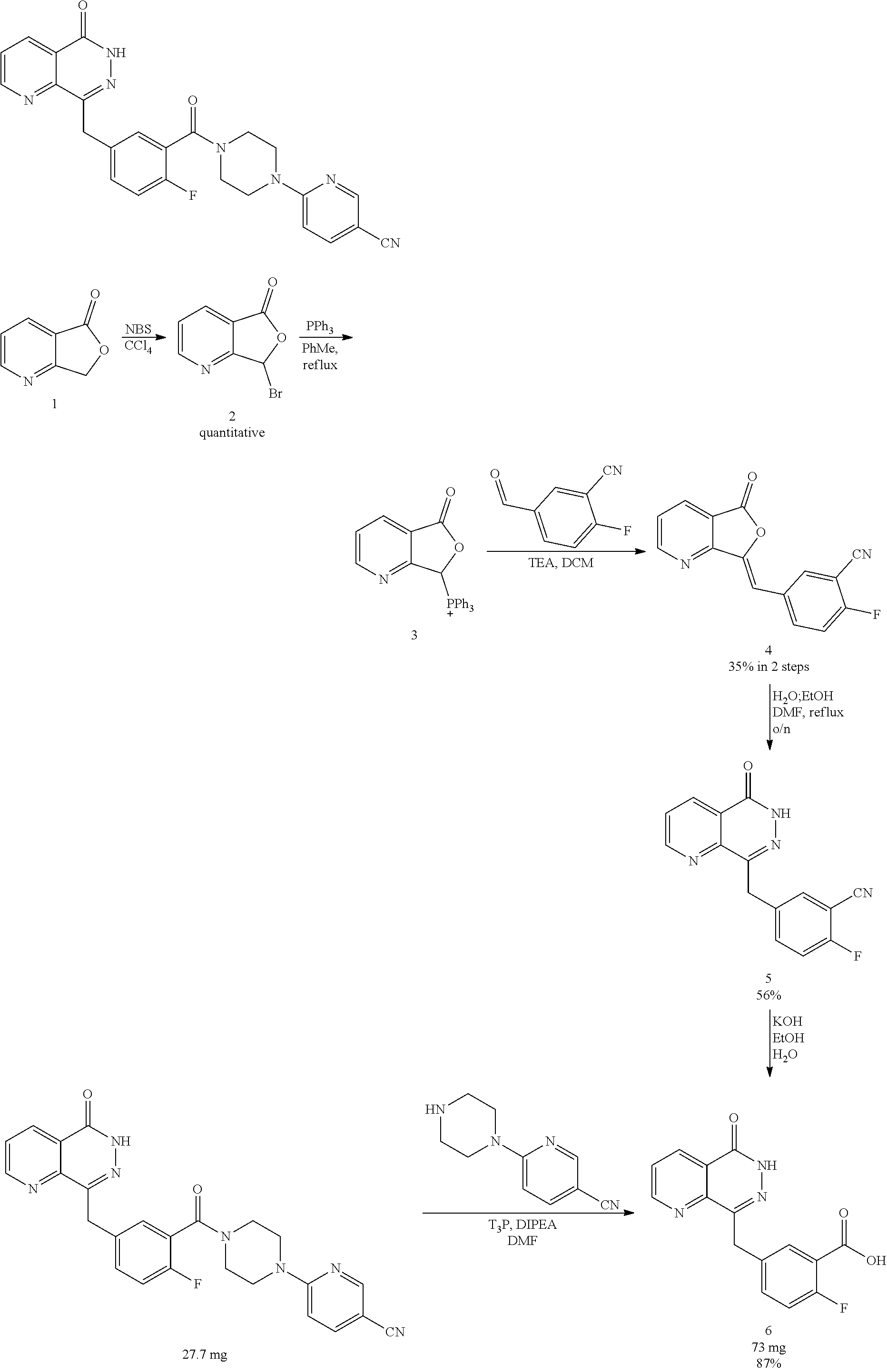

1 (0.405 g, 3.0) mmol) and 1-bromopyrrolidine-2,5-dione (0.59 g, 3.3 mmol) were dissolved CCI4 and heated at reflux for 2 hours. The reaction was cooled and filtered, the filtrate was evaporated to afford 7-bromofuro[3,4-b]pyridin-5(7H)-one (2), as a yellow solid (quantitative yield), which was used directly in the next stage without further purification.

2 (3.0, 0.642 mmol) and triphenylphosphine (0.86 g, 3.3 mmol) were heated at reflux in acetonitrile (20 mL) for 4 h. The reaction was cooled and concentrated and purified by using flash column (DCM/MeOH) to afford the desired material 3.

3 (0.605 g, 1.27 mmol) and 2-fluoro-5-formylbenzonitrile (0.23 g, 1.52 mmol) were dissolved DCM (7 mL), to this was added triethylamine (0.27 mL, 1.9 mmol) and the reaction was stirred overnight. The reaction mixture was quenched with water (50 mL), extracted with EA (2×15 mL), the organic layer was dried over MgSO4, filtered and evaporated to afford an orange gum crude. This was passed through a plug of silica eluting with ethyl acetate to afford 4 as a pale yellow oil. (0.13 g, 35%)

To the above crude 4 (0.13 g) was added water (2 mL), EtOH (2 mL) and DMF (0.12 mL). Hydrazine hydrate (0.16 mL) were added and the reaction was heated at reflux overnight. The reaction was cooled, and the precipitate was collected by filtration, washed with EtOH (25 mL) and air dried to afford the desired compound 5 as a white solid (78.2 mg, 56%)

To 5 (78.2 mg) were added KOH (0.15 g, 10.0 mmol) and EtOH (1 mL) and water (3.5 mL) and heated at 100° C. for 5 hours. The ethanol was evaporated off and the aqueous was extracted with ethyl acetate (2×5 mL). The aqueous was then acidified to pH1 with cone HCl to afford a solid, which was filtered, washed with water and dried to the desired material 6 as a beige solid. (73 mg, 87%)

2-fluoro-5-((5-oxo-5,6-dihydropyrido[2,3-d]pyridazin-8-yl)methyl)benzoic acid (0.028 g, 0.069 mmol) and 6-(piperazin-1-yl)nicotinonitrile (0.016 g, 0.083 mmol, 1.2 equiv.) were dissolved in anhydrous DMF (0.4 mL) and cooled to 0° C. To this mixture was added diisopropylethylamine (35 uL, 3.0 equiv.) followed by addition of propylphosphonic anhydride (T3P) (90 uL, 0.13 mmol, 3.0 equiv.) (50% in EtOAc). The mixture was stirred for 30 min at rt. The mixture was diluted with ethyl acetate (5 mL) and sat. NaHCO₃ (5 mL). The layers were separated and the aqueous layer was extracted with ethyl acetate. The organic extracts were washed with brine (5 mL), filtered through a silica plug, and dried over MgSO₄. After filtration, the solvent was removed in vacuo. Flash chromatography (0-100% EtOAc in Heptanes) over silica gel afforded the product as a white solid (0.0277 g). LCMS ESI m/z: 470.1 [M+H]⁺. ¹H NMR (400 MHz, CDCl₃) δ 10.87 (s, 1H), 9.10 (dd, J=4.6, 1.8 Hz, 1H), 8.69 (dd, J=8.1, 1.8 Hz, 1H), 8.44-8.39 (m, 1H), 7.76-7.62 (m, 2H), 7.51 (ddd, J=13.2, 5.0, 2.2 Hz, 2H), 7.03 (t, J=8.8 Hz, 1H), 6.62 (d, J=9.0 Hz, 1H), 4.45 (s, 2H), 3.89 (s, 2H), 3.84-3.72 (m, 2H), 3.44 (s, 2H).

### Synthesis of Example 93: 6-(4-(5-((7-Cyclobutoxy-4-oxo-5-(trifluoromethyl)-3,4-dihydrophthalazin-1-yl)methyl)-2-fluorobenzoyl)piperazin-1-yl)nicotinonitrile

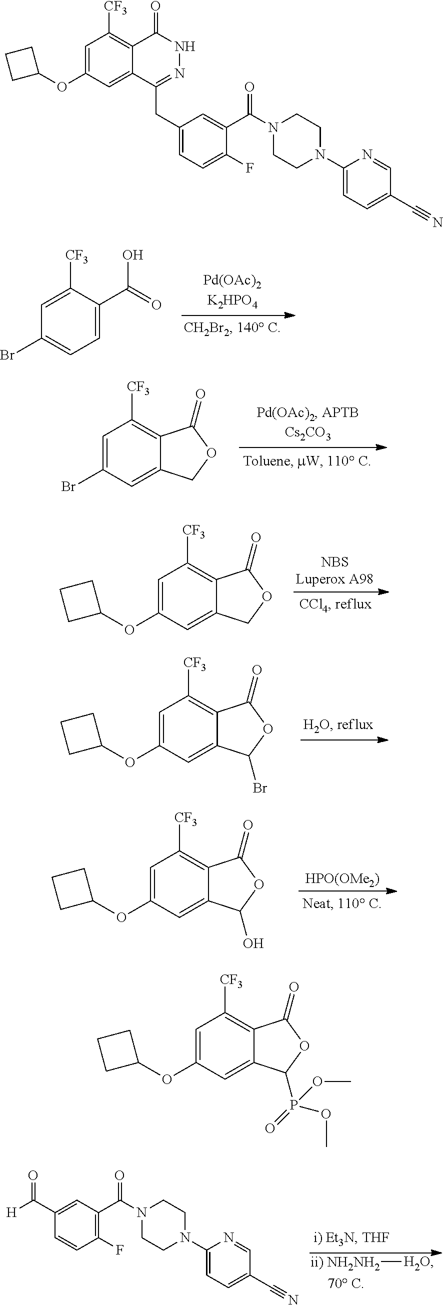

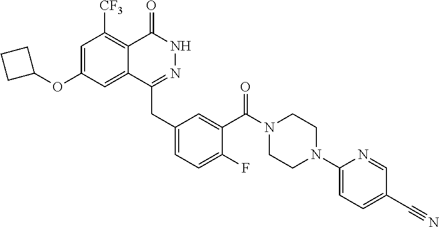 [SMILES: N#Cc1ccc(N2CCN(C(=O)c3cc(Cc4n[nH]c(=O)c5c(C(F)(F)F)cc(OC6CCC6)cc45)ccc3F)CC2)nc1]

### Step 1

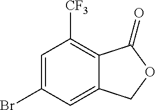 [SMILES: O=C1OCc2cc(Br)cc(C(F)(F)F)c21]

### 5-Bromo-7-(trifluoromethyl)isobenzofuran-1(3H)-one

To a high pressure reaction vessel was added 4-bromo-2-(trifluoromethyl)benzoic acid (1.80 g, 6.49 mmol), Pd(OAc)₂ (729 mg, 3.25 mmol), potassium phosphate dibasic (3.46 g, 19.5 mmol), and finally methylene bromide (14 mL, 6.49 mmol). The tube was sealed and the reaction was heated to 140° C. for 24 hours. The crude reaction mixture was filtered through a 0.45 n syringe filter and the resulting filtrate was concentrated under vacuum. The resulting residue was dissolved in minimal DCM and subjected to normal phase chromotography (Heptanes-EtOAc, 5% to 100% over 10 minutes). The collected fractions were concentrated under vacuum to afford 5-bromo-7-(trifluoromethyl)isobenzofuran-1(3H)-one (630 mg, 35%) as a white solid. ¹H NMR (400 MHz, DMSO-d₆) δ 8.31 (s, 1H), 8.13 (s, 1H), 5.45 (s, 2H).

### Step 2

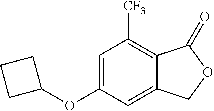 [SMILES: O=C1OCc2cc(OC3CCC3)cc(C(F)(F)F)c21]

### 5-Cyclobutoxy-7-(trifluoromethyl)isobenzofuran-1(3H)-one

A flame-dried vial with stir bar was charged with 5-bromo-7-(trifluoromethyl)isobenzofuran-1(3H)-one (606 mg, 2.16 mmol). Pd(OAc)₂ (19.8 mg, 86.3 umol), Cs₂CO₃ (1.42 g, 4.31 mmol) and 5-[di(1-adamantyl)phosphino]-12,32,52-triphenyl-12H-[1,42]bipyrazole (114 mg, 173 umol). The vial was sealed with a septum and purged with nitrogen gas. A separate screw cap vial was charged with toluene (3.72 mL) and cyclobutanol (517 uL, 6.47 mmol), capped and degassed for a 5 minutes period using a nitrogen balloon and sonication. The mixture was then added to the vial under nitrogen and the reaction mixture was stirred at 100° C. for 24 hours. After 20 hours of stirring, the reaction mixture then cooled and filtered. The filtrate was then concentrated under vacuum and the resulting residue was suspended in minimal DCM and subjected to normal-phase chromotography (5%4-95% Heptanes:EtOAc) to afford 5-cyclobutoxy-7-(trifluoromethyl)isobenzofuran-1(3H)-one (110 mg, 19% yield) as a white solid. LCMS ESI m/z: 273.2 [M+H]⁺.

### Step 3

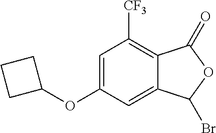 [SMILES: O=C1OC(Br)c2cc(OC3CCC3)cc(C(F)(F)F)c21]

### 3-Bromo-5-cyclobutoxy-7-(trifluoromethyl)isobenzofuran-1(3H)-one

To a solution of cyclobutoxy-7-(trifluoromethyl)isobenzofuran-1(3H)-one (110 mg, 0.404 mmol) in CCl₄ (1.99 mL) was added n-bromosuccinimide (72.6 mg, 0.404 mmol) and finally Luperox A98, benzoyl peroxide (4.31 uL, 0.0202 mmol). The solution was refluxed for 24 hours. After completion, the reaction was concentrated under vacuum and the resulting solution was dissolved in minimal DCM and subjected to normal phase chromotography (Heptanes:EtOAc, 5-95%) to afford 3-bromo-5-cyclobutoxy-7-(trifluoromethyl)isobenzofuran-1(3H)-one (142 mg, 100% yield) as an off-white solid which was carried forward to the next step. LCMS ESI m/z: 367 [M+OH]⁻.

### Step 4

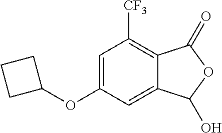 [SMILES: O=C1OC(O)c2cc(OC3CCC3)cc(C(F)(F)F)c21]

### 5-Cyclobutoxy-3-hydroxy-7-(trifluoromethyl)isobenzofuran-1(3H)-one

A mixture of 3-bromo-5-cyclobutoxy-7-(trifluoromethyl)isobenzofuran-1(3H)-one (142 mg, 404 umol) in H₂O (5 mL) was stirred at reflux for 16 hours. The reaction was cooled to room and extracted with DCM (10 mL×3). The combined organic layer was dried over anhydrous Na₂SO₄, filtered and concentrated in vacuum to afford 5-cyclobutoxy-3-hydroxy-7-(trifluoromethyl)isobenzofuran-1(3H)-one (117 mg, 100% yield) as a light-yellow solid which was carried forward. LCMS ESI m/z: 287.1 [M−H]⁺.

### Step 5

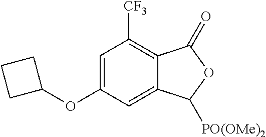

### Dimethyl (6-cyclobutoxy-3-oxo-4-(trifluoromethyl)-1,3-dihydroisobenzofuran-1-yl)phosphonate

To a microwave vial was added 5-cyclobutoxy-3-hydroxy-7-(trifluoromethyl)isobenzofuran-1(3H)-one (117 mg, 406 umol) and dimethyl phosphite (1.00 mL). The solution was sparged with nitrogen and the reaction was stirred at 110° C. for 45 minutes. The reaction was then diluted with water and EtOAc and extracted three times. The pooled organic layers were then washed with water and finally brine. The organic layer was then concentrated under vacuum to afford a yellow oil. The oil was left under high vacuum overnight to afford dimethyl (6-cyclobutoxy-3-oxo-4-(trifluoromethyl)-1,3-dihydroisobenzofuran-1-yl)phosphonate (154 mg, 100% yield) as an off-white solid. LCMS ESI m/z: 381.2 [M−H]⁺.

### Step 6

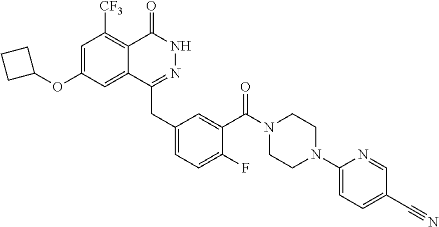 [SMILES: N#Cc1ccc(N2CCN(C(=O)c3cc(Cc4n[nH]c(=O)c5c(C(F)(F)F)cc(OC6CCC6)cc45)ccc3F)CC2)nc1]

### 6-(4-(5-((7-cyclobutoxy-4-oxo-5-(trifluoromethyl)-3,4-dihydrophthalazin-1-yl)methyl)-2-fluorobenzoyl)piperazin-1-yl)nicotinonitrile

To a glass vial containing (6-cyclobutoxy-3-oxo-4-(trifluoromethyl)-1,3-dihydroisobenzofuran-1-yl)phosphonate (163 mg, 429 umol) in THF (21.4 mL) was added triethylamine (179 uL, 1.29 mmol) and 6-(4-(2-fluoro-5-formylbenzoyl)piperazin-1-yl)nicotinonitrile (160 mg, 472 umol). The reaction was heated to 70° C. for 48 hours. Hydrazine hydrate solution (54.4 uL, 1.71 mmol) was added and the reaction was left to stir for another 48 hours at 70° C. After completion of the reaction, the solution was concentrated under vacuum. Methanol was then added to the resulting residue which induced the formation of a white solid. The solid was filtered off and the eluent was again concentrated. The residue was dissolved in minimal DMSO and subjected to reverse phase chromatography (10 mM aqueous ammonium formate:MeCN, 5-95%). The fractions containing product were pooled and lyophilized to afford 6-(4-(5-((7-cyclobutoxy-4-oxo-5-(trifluoromethyl)-3,4-dihydrophthalazin-1-yl)methyl)-2-fluorobenzoyl)piperazin-1-yl)nicotinonitrile (8.20 mg, 3.2% yield) as a white powder. LCMS ESI m/z: 607.4 [M+H]¹H NMR (400 MHz, DMSO-D6) δ 12.64 (s, 1H), 8.51 (d, J=2.2 Hz, 1H), 7.90 (dd, J=9.1, 2.3 Hz, 1H), 7.62 (d, J=2.0 Hz, 1H), 7.45-7.39 (m, 2H), 7.32-7.23 (m, 2H), 6.93 (d, J=9.1 Hz, 1H), 4.93 (p. J=7.1 Hz, 1H), 4.37 (s, 2H), 3.75 (s, 2H), 3.72 (s, 2H), 3.60 (s, 2H), 3.29 (s, 2H), 2.34 (d, J=7.5 Hz, 2H), 2.05-1.93 (m, 2H), 1.78 (q, J=10.2 Hz, 1H), 1.66 (dd, J=18.6, 8.5 Hz, 1H).

### Synthesis of Example 94: 6-(4-(5-((7-Cyclobutoxy-4-oxo-3,4-dihydrophthalazin-1-yl)methyl)-2-fluorobenzoyl)-1,4-diazepan-1-yl)nicotinonitrile

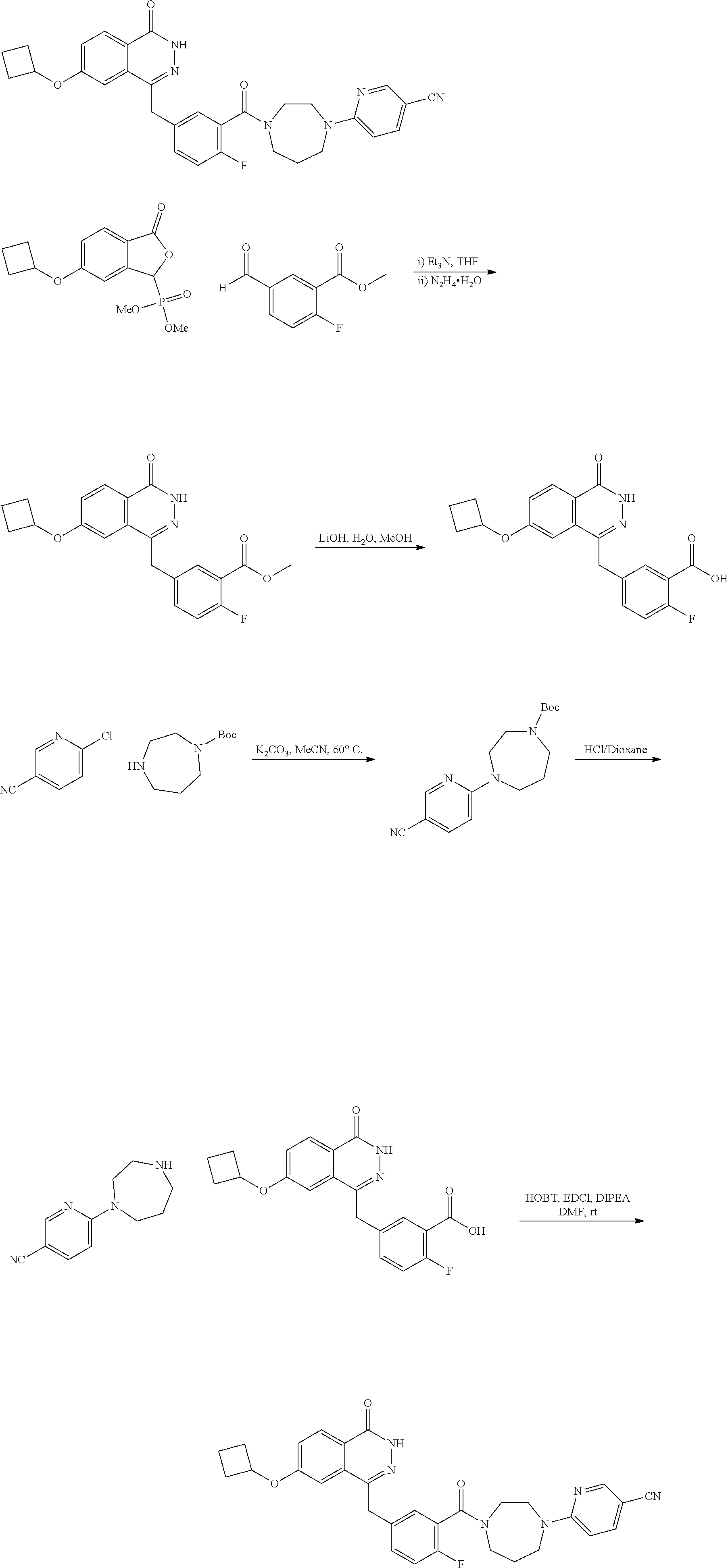

### Step 1

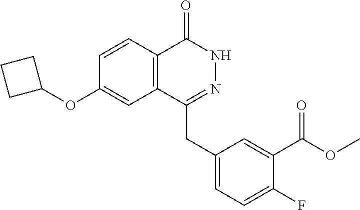 [SMILES: COC(=O)c1cc(Cc2n[nH]c(=O)c3ccc(OC4CCC4)cc23)ccc1F]

### Methyl 5-((7-cyclobutoxy-4-oxo-3,4-dihydrophthalazin-1-yl)methyl)-2-fluorobenzoate

A solution of dimethyl (6-cyclobutoxy-3-oxo-1,3-dihydroisobenzofuran-1-yl)phosphonate (1.6 g, 4.20 mmol), methyl 2-fluoro-5-formylbenzoate (762 mg, 4.20 mmol) and Et₃N (7010 mg, 6.9 mmol) in anhydrous THF (10 mL) was stirred at 60° C. under Ar (g) for 15 hours. Hydrazine hydrate (202 mg, 6.3 mmol) was added and the reaction was heated to 70° C. for 1 hour. The reaction was concentrated under vacuum. The formed solids were washed with water (10 mL) and pet ether/EA (2:1, 10 mL) to afford methyl 5-((7-cyclobutoxy-4-oxo-3,4-dihydrophthalazin-1-yl)methyl)-2-fluorobenzoate as a white solid (1.16 g, 59% yield). LCMS EST m/z 383 [M+H]⁺.

### Step 2

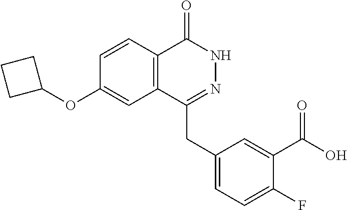 [SMILES: O=C(O)c1cc(Cc2n[nH]c(=O)c3ccc(OC4CCC4)cc23)ccc1F]

### 5-((7-Cyclobutoxy-4-oxo-3,4-dihydrophthalazin-1-yl)methyl)-2-fluorobenzoic aciD

A mixture solution of methyl 5-((7-cyclobutoxy-4-oxo-3,4-dihydrophthalazin-1-yl)methyl)-2-fluorobenzoate (1.16 g, 3.03 mmol) and LiOH—H₂O (636 mg, 15.15 mmol) in MeOH (15 mL) and H₂O (5 mL) was stirred at rt for 3 hours. The organic solvent was removed under vacuo and aq HCl (1N) was added until the solution reached pH 5˜6. The solids were collected by filtration, washed with water and dried under vacuum to give 5-((7-cyclobutoxy-4-oxo-3,4-dihydrophthalazin-1-yl)methyl)-2-fluorobenzoic acid as a white solid (1.05 g, 89% yield). This was used without further purification. LCMS ESI m/z 369 [M+H]⁺.

### Step 3

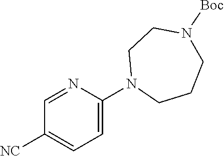 [SMILES: CC(C)(C)OC(=O)N1CCCN(c2ccc(C#N)cn2)CC1]

### tert-Butyl 4-(5-cyanopyridin-2-yl)-1,4-diazepane-1-carboxylate

A mixture of 6-chloronicotinonitrile (300 mg, 2.17 mmol), tert-butyl 1,4-diazepane-1-carboxylate (434 mg, 2.17 mmol) and K₂CO₃ (599 mg, 4.34 mmol) in ACN (5 mL) was stirred at 60° C. for 16 hours. The reaction was concentrated in vacuo, diluted with water (20 mL) and extracted with EtOAc (20 mL×3). The combined organic layer was washed with brine, dried over anhydrous Na₂SO₄, filtered and concentrated in vacuo. The residue was purified by silica gel chromatography (EtOAc/petroleum ether=10% to 50%) to give tert-butyl 4-(5-cyanopyridin-2-yl)-1,4-diazepane-1-carboxylate (620 mg, 95% yield) as a white solid. LCMS EST m/z 303 [M+H]⁺.

### Step 4

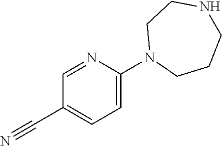 [SMILES: N#Cc1ccc(N2CCCNCC2)nc1]

### 6-(1,4-Diazepan-1-yl)nicotinonitrile

A solution of tert-butyl 4-(5-cyanopyridin-2-yl)-1,4-diazepane-1-carboxylate (620 mg, 2.05 mmol) in HCU/dioxane (4.0 M, 10 mL, 40.0 mmol) was stirred at rt for 1 hour. The solvent was removed in vacuo to give 6-(azetidin-3-yl(methyl)amino)nicotinonitrile (400 mg, 96% yield) as a white solid. This was used without further purification. LCMS ESI m/z: 203 [M+H]⁺.

### Step 5

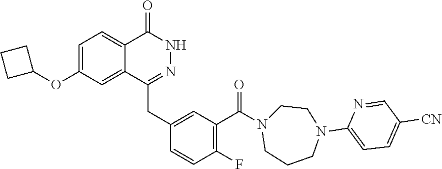 [SMILES: N#Cc1ccc(N2CCCN(C(=O)c3cc(Cc4n[nH]c(=O)c5ccc(OC6CCC6)cc45)ccc3F)CC2)nc1]

### 6-(4-(5-((7-Cyclobutoxy-4-oxo-3,4-dihydrophthalazin-1-yl)methyl)-2-fluorobenzoyl)-1,4-diazepan-1-yl)nicotinonitrile

To a solution of 5-((7-cyclobutoxy-4-oxo-3,4-dihydrophthalazin-1-yl)methyl)-2-fluorobenzoic acid (80 mg, 0.22 mmol) and 6-(1,4-diazepan-1-yl)nicotinonitrile (62 mg, 0.26 mmol) in DMF (2 mL) was added EDCI (62 mg, 0.33 mmol). HOBt (44 mg, 0.33 mmol) and DIPEA (140 mg, 1.10 mmol). The reaction mixture was stirred at rt for 2 hours. The reaction was purified by prep-HPLC to give 6-(4-(5-((7-cyclobutoxy-4-oxo-3,4-dihydrophthalazin-1-yl)methyl)-2-fluorobenzoyl)-1,4-diazepan-1-yl)nicotinonitrile (52 mg, 43% yield) as a white solid. LCMS EST m/z: 553 [M+H]⁺. ¹H NMR (400 MHz, DMSO-d₆) δ 12.44 (s, 1H), 8.50-8.30 (m, 1H), 8.19-8.12 (m, 1H), 7.87-7.80 (m, 1H), 7.43-7.28 (m, 2H), 7.26-7.11 (m, 1H), 7.11-7.06 (m, 1H), 7.05-6.74 (m, 2H), 4.87-4.75 (m, 1H), 4.25 (d, J=24.2 Hz, 2H), 3.91-3.47 (m, 7H), 3.28-3.14 (m, 1H), 2.42-2.32 (m, 2H), 2.05-1.94 (m, 2H), 1.89-1.37 (m, 4H).

### Synthesis of Example 95: 2-(4-(5-((7-Cyclobutoxy-4-oxo-3,4-dihydrophthalazin-1-yl)methyl)-2-fluorobenzoyl)piperazin-1-yl)isonicotinonitrile

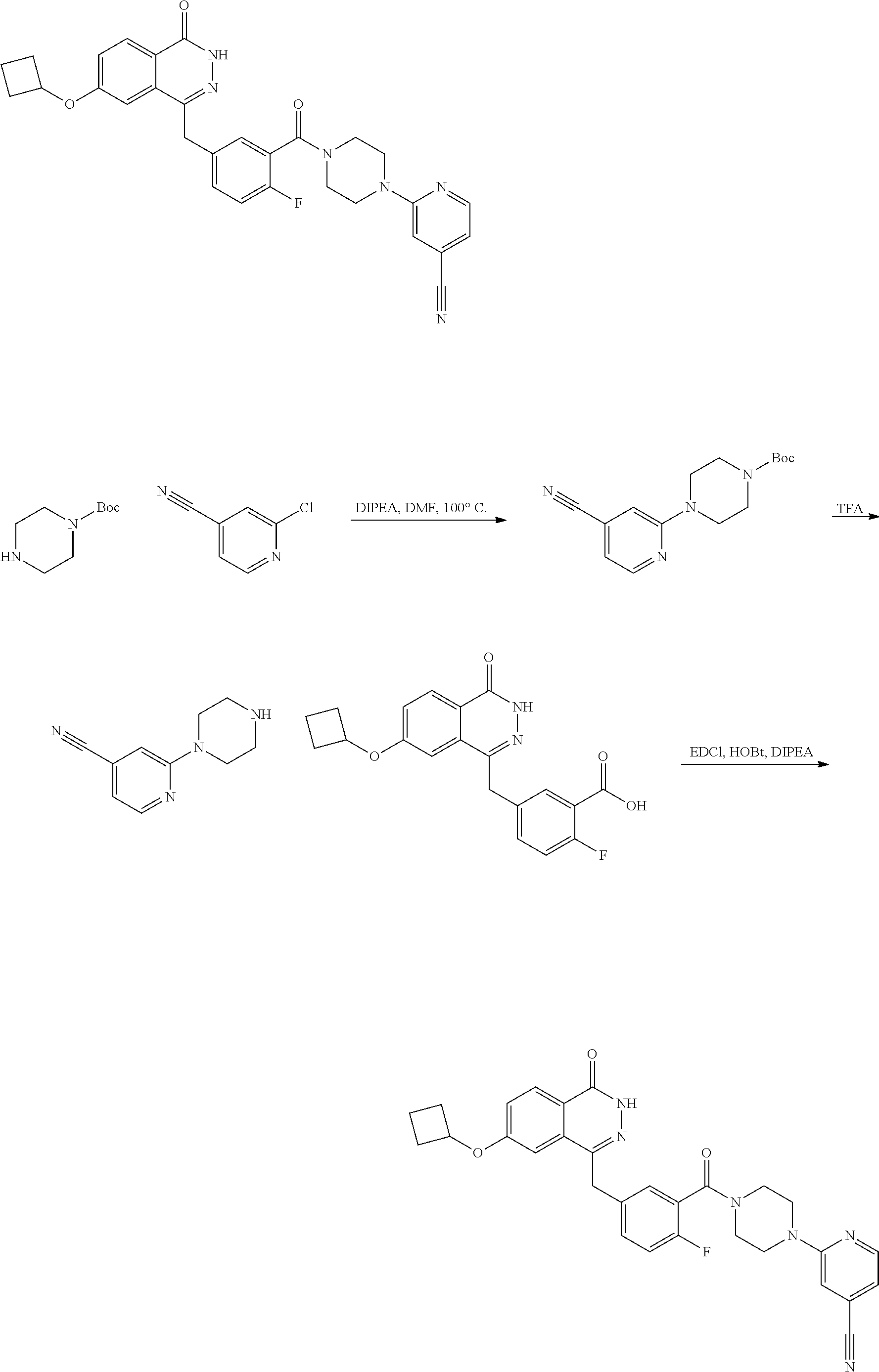

### Step 1

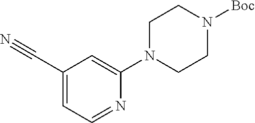 [SMILES: CC(C)(C)OC(=O)N1CCN(c2cc(C#N)ccn2)CC1]

### tert-Butyl 4-(4-cyanopyridin-2-yl)piperazine-1-carboxylate

A mixture of 2-chloroisonicotinonitrile (200 mg, 1.44 mmol), tert-butyl piperazine-1-carboxylate (323 mg, 1.73 mmol) and DIPEA (746 mg, 5.77 mmol) in DMF (5 mL) was stirred at 100° C. for 20 hours. The mixture was cooled to rt, diluted with H₂O (10 mL) and extracted with EtOAc (10 mL×3). The combined organic layer was washed with brine, dried over anhydrous Na₂SO₄, filtered and concentrated in vacuo. The residue was purified by silica gel chromatography (EtOAc/pet ether=0 to 10%) to give tert-butyl 4-(4-cyanopyridin-2-yl)piperazine-1-carboxylate as a white solid (350 mg, 84% yield). LCMS ESI m/z 289 [M+H]⁺.

### Step 2

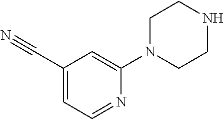 [SMILES: N#Cc1ccnc(N2CCNCC2)c1]

### 2-(Piperazin-1-yl)isonicotinonitrile

A solution of tert-butyl 4-(4-cyanopyridin-2-yl)piperazine-1-carboxylate (350 mg, 1.21 mmol) in DCM (5 mL) and TFA (0.5 mL) was stirred at 25° C. for 2 hours. The reaction mixture was concentrated in vacuo to afford 2-(piperazin-1-yl)isonicotinonitrile as a yellow oil (366 mg, 99% yield). This was used without further purification. LCMS ESI m/z 189 [M+H]⁺.

### Step 3

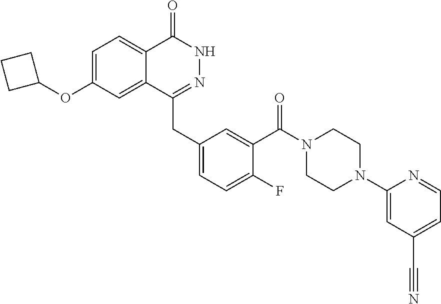 [SMILES: N#Cc1ccnc(N2CCN(C(=O)c3cc(Cc4n[nH]c(=O)c5ccc(OC6CCC6)cc45)ccc3F)CC2)c1]

### 2-(4-(5-((7-Cyclobutoxy-4-oxo-3,4-dihydrophthalazin-1-yl)methyl)-2-fluorobenzoyl)piperazin-1-yl)isonicotinonitrile

To a solution of 2-(piperazin-1-yl)isonicotinonitrile (43 mg, 0.23 mmol) and 5-((7-cyclobutoxy-4-oxo-3,4-dihydrophthalazin-1-yl)methyl)-2-fluorobenzoic acid (70 mg, 0.19 mmol) in DMF (3 mL) was added EDCI (36 mg, 0.19 mmol), HOBt (39 mg, 0.29 mmol) and DIPEA (74 mg, 0.57 mmol). The reaction mixture was stirred at rt for 16 hours. The mixture was diluted with H₂O (10 mL) and extracted with EtOAc (10 mL×3). The combined organic layer was concentrated in vacuo and the residue was purified by prep-HPLC to give 2-(4-(5-((7-cyclobutoxy-4-oxo-3,4-dihydrophthalazin-1-yl)methyl)-2-fluorobenzoyl)piperazin-1-yl)isonicotinonitrile as a white solid (55 mg, 54% yield). LCMS ESI m/z 539 [M+H]⁺. ¹H NMR (400 MHz, DMSO-d₆) δ 12.45 (s, 1H), 8.31 (d, J=5.0 Hz, 1H), 8.15 (d, J=8.8 Hz, 1H), 7.42 (t, J=6.4 Hz, 2H), 7.34-7.22 (m, 3H), 7.11 (d, J=2.3 Hz, 1H), 7.00 (dd, J=5.0, 1.0 Hz, 1H), 4.91-4.76 (m, 1H), 4.31 (s, 2H), 3.77-3.62 (m, 4H), 3.52 (s, 2H), 3.29 (s, 2H), 2.44-2.34 (m, 2H), 2.06-1.95 (m, 2H), 1.83-1.58 (m, 2H).

### Synthesis of Example 96: 6-(8-(5-((7-Cyclobutoxy-4-oxo-3,4-dihydrophthalazin-1-yl)methyl)-2-fluorobenzoyl)-3,8-diazabicyclo[3.2.1]octan-3-yl)nicotinonitrile

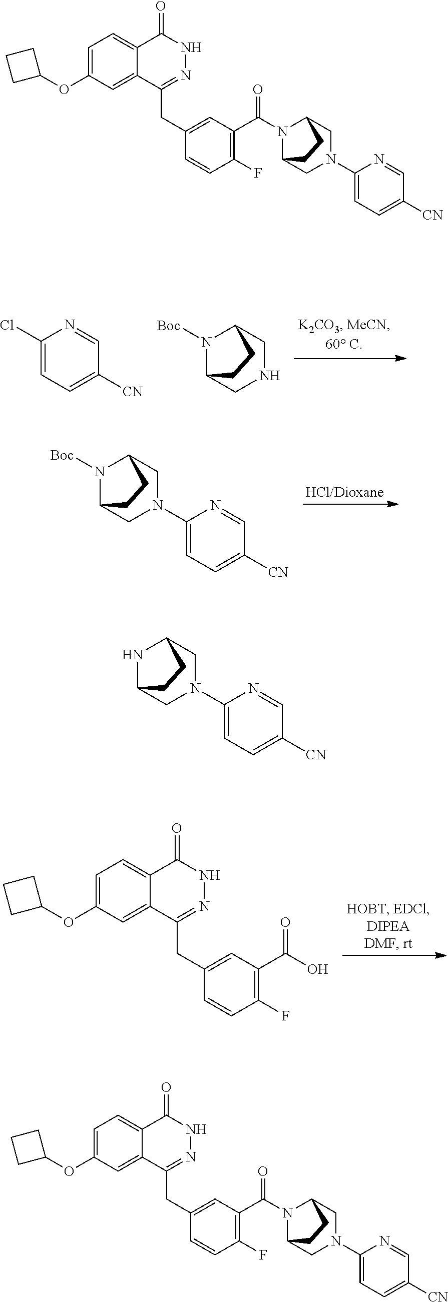

### Step 1

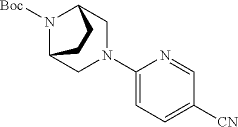

### tert-Butyl 3-(5-cyanopyridin-2-yl)-3,8-diazabicyclo[3.2.1]octane-8-carboxylate

A mixture of 6-chloronicotinonitrile (300 mg, 2.17 mmol), tert-butyl 3,8-diazabicyclo[3.2.1]octane-8-carboxylate (460 mg, 2.17 mmol) and K₂CO₃ (509 mg, 3.68 mmol) in MeCN (5 mL) was stirred at 60° C. for 16 hours. The reaction was concentrated in vacuo, diluted with water (20 mL) and extracted with EtOAc (20 mL×3). The combined organic layer was washed with brine, dried over anhydrous Na₂SO₄, filtered and concentrated in vacuo. The residue was purified by silica gel chromatography (EtOAc/petroleum ether=10% to 50%) to give tert-butyl 3-(5-cyanopyridin-2-yl)-3,8-diazabicyclo[3.2.1]octane-8-carboxylate (580 mg, 85% yield) as a white solid. LCMS ESI m/z: 315 [M+H]⁺.

### Step 2

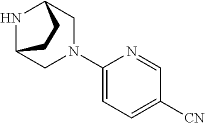

### 6-(3,8-Diazabicyclo[3.2.1]octan-3-yl)nicotinonitrile

A solution of tert-butyl 3-(5-cyanopyridin-2-yl)-3,8-diazabicyclo[3.2.1]octane-8-carboxylate (580 mg, 1.84 mmol) in HCl/dioxane (4.0M, 5 mL, 20.0 mmol) was stirred at rt for 1 hour. The solvent was removed in vacuo to give 6-(3,8-diazabicyclo[3.2.1]octan-3-yl)nicotinonitrile (370 mg, 94% yield) as a white solid. This was used without further purification. LCMS ESI m/z: 215 [M+H]⁺.

### Step 3

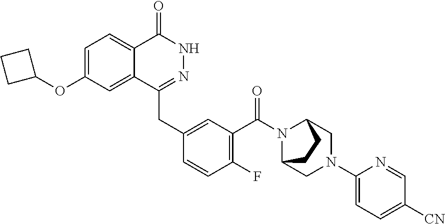

### 6-(8-(5-((7-Cyclobutoxy-4-oxo-3,4-dihydrophthalazin-1-yl)methyl)-2-fluorobenzoyl)-3,8-diazabicyclo[3.2.1]octan-3-yl)nicotinonitrile

To a solution of 5-((7-cyclobutoxy-4-oxo-3,4-dihydrophthalazin-1-yl)methyl)-2-fluorobenzoic acid (80 mg, 0.22 mmol) and 6-(3,8-diazabicyclo[3.2.1]octan-3-yl)nicotinonitrile (163 mg, 0.66 mmol) in DMF (2 mL) was added EDCI (62 mg, 0.33 mmol), HOBt (44 mg, 0.33 mmol) and DIPEA (140 mg, 1.10 mmol). The reaction mixture was stirred at rt for 2 h. The reaction was purified by prep-HPLC to give 6-(8-(5-((7-cyclobutoxy-4-oxo-3,4-dihydrophthalazin-1-yl)methyl)-2-fluorobenzoyl)-3,8-diazabicyclo[3.2.1]octan-3-yl)nicotinonitrile (38 mg, 31% yield) as a white solid. LCMS ESI m/z: 565 [M+H]⁺. ¹H NMR (400 MHz, DMSO-d₆) δ 12.45 (s, 1H), 8.52-8.50 (m, 1H), 8.16 (d, J=8.8 Hz, 1H), 7.92-7.88 (m, 1H), 7.51-7.47 (m, 1H), 7.46-7.41 (m, 1H), 7.33-7.24 (m, 2H), 7.13-7.10 (m, 1H), 6.85 (d, J=9.2 Hz, 1H), 4.87-4.78 (m, 2H), 4.32 (s, 2H), 4.25 (d, J=12.4 Hz, 1H), 4.08 (d, J=12.1 Hz, 1H), 3.88-3.82 (m, 1H), 3.13 (d, J=11.7 Hz, 1H), 2.90 (d, J=12.1 Hz, 1H), 2.43-2.36 (m, 2H), 2.03-1.95 (m, 2H), 1.91-1.79 (m, 2H), 1.78-1.61 (m, 4H).

### Synthesis of Example 97: 6-cyclobutoxy-4-(4-fluoro-3-(3-(5-(trifluoromethyl)pyrimidin-2-yl)-3,8-diazabicyclo[3.2.1]octane-8-carbonyl)benzyl)phthalazin-1(2H)-one

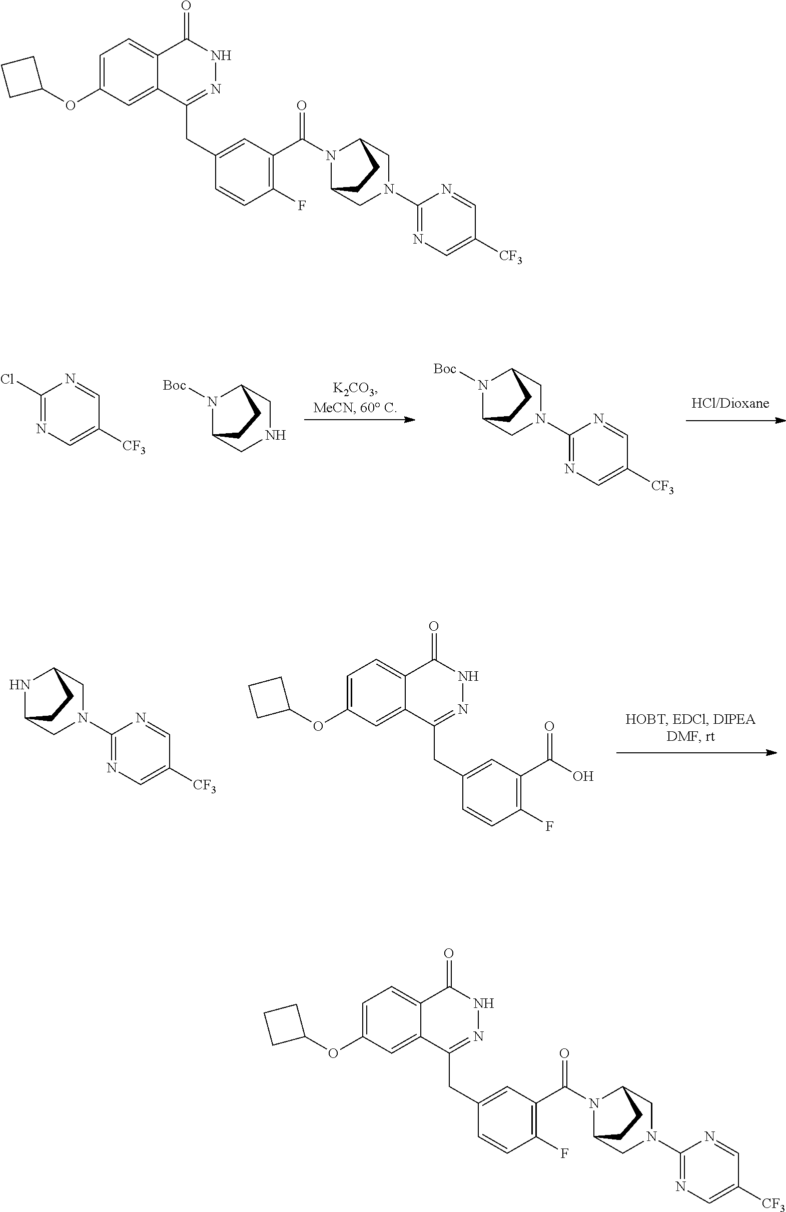

### Step 1

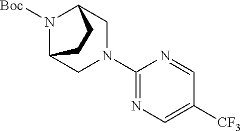

### tert-Butyl 3-(5-(trifluoromethyl)pyrimidin-2-yl)-3,8-diazabicyclo[3.2.1]octane-8-carboxylate

A mixture of tert-butyl 3,8-diazabicyclo[3.2.1]octane-8-carboxylate (200 mg, 0.94 mmol), 2-chloro-5-(trifluoromethyl)pyrimidine (172 mg, 0.94 mmol) and K₂CO₃ (221 mg, 0.02 mmol) in ACN (4 mL) was stirred at 60° C. for 12 hours. The solid was filtered off, and the filtrate was concentrated in vacuo. The residue was purified by silica gel chromatography (EtOAc/pet ether=40% to 50%) to give the desired product tert-butyl 3-(5-(trifluoromethyl)pyrimidin-2-yl)-3,8-diazabicyclo[3.2.1]octane-8-carboxylate (302 mg, 89% yield) as a white solid. LCMS ESI m: 359.1 [M+H]⁺.

### Step 2

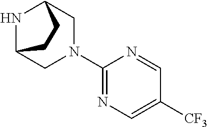

### 3-(5-(trifluoromethyl)pyrimidin-2-yl)-3,8-diazabicyclo)[3.2.1]octane

To a solution of tert-butyl 3-(5-(trifluoromethyl)pyrimidin-2-yl)-3,8-diazabicyclo[3.2.1]octane-8-carboxylate (300 mg, 0.84 mmol) in dioxane (1 mL) was added HCl/dioxane (4.0M in dioxane, 2 mL, 8.0 mmol). The mixture was stirred at rt for 1 hour, then concentrated in vacuo to afford 3-(5-(trifluoromethyl)pyrimidin-2-yl)-3,8-diazabicyclo[3.2.1]octane (193 mg, 89% yield) as a white solid. This was used without further purification. LCMS ESI m/z 259.1 [M+H]⁺.

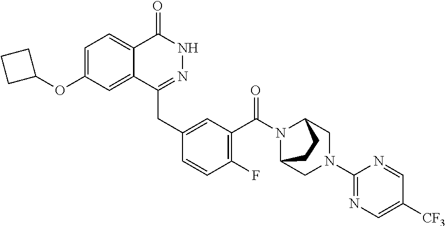

### 6-cyclobutoxy-4-(4-fluoro-3-(3-(5-(trifluoromethyl)pyrimidin-2-yl)-3,8-diazabicyclo[3.2.1]octane-8-carbonyl)benzyl)phthalazin-1(2H)-one

To a solution of 5-((7-cyclobutoxy-4-oxo-3,4-dihydrophthalazin-1-yl)methyl)-2-fluorobenzoic acid (80 mg, 0.21 mmol) and 3-(5-(trifluoromethyl)pyrimidin-2-yl)-3,8-diazabicyclo[3.2.1]octane (76.8 mg, 0.40 mmol) in DMF (2 mL) was added EDC (63 mg, 0.32 mmol). HOBt (44 mg, 0.32 mmol) and DIPEA (140 mg, 1.09 mmol). The reaction mixture was stirred at rt for 2 hours. The reaction was purified by prep-HPLC to give 6-cyclobutoxy-4-(4-fluoro-3-(3-(5-(trifluoromethyl)pyrimidin-2-yl)-3,8-diazabicyclo[3.2.1]octane-8-carbonyl)benzyl)phthalazin-1(2H)-one (45 mg, 34% yield) as a white solid. LCMS EST 609.2 [M+H]⁺. ¹H NMR (400 MHz, DMSO-d6) δ 12.46 (s, 1H), 8.73 (s, 1H), 8.26-8.06 (m, 1H), 7.50 (dd, J=6.4, 2.0 Hz, 1H), 7.46-7.40 (m, 1H), 7.34-7.24 (m, 2H), 7.11 (d, J=2.3 Hz, 1H), 4.84 (dd, J=14.1, 6.9 Hz, 2H), 4.67-4.23 (m, 4H), 3.87 (d, J=5.2 Hz, 1H), 3.10 (dd, J=86.6, 12.5 Hz, 2H), 2.38 (m, J=25.4, 12.7 Hz, 2H), 2.07-1.43 (m, 8H).

### Synthesis of Example 98: 6-Cyclobutoxy-4-(4-fluoro-3-(4-(5-(trifluoromethyl)pyrimidin-2-yl)-1,4-diazepane-1-carbonyl)benzyl)phthalazin-1(2H)-one

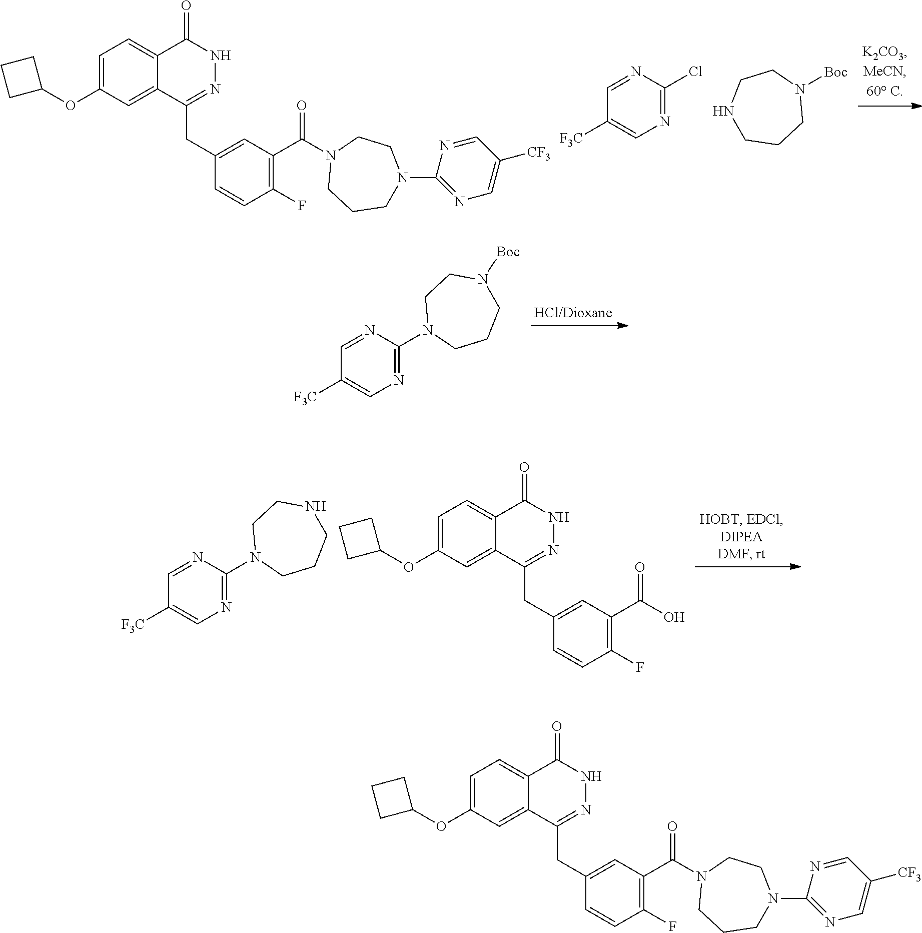

### Step 1

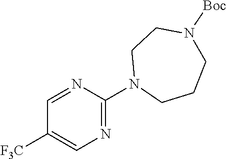 [SMILES: CC(C)(C)OC(=O)N1CCCN(c2ncc(C(F)(F)F)cn2)CC1]

### tert-Butyl 4-(5-(trifluoromethyl)pyrimidin-2-yl)-1,4-diazepane-1-carboxylate

A mixture of tert-butyl 1,4-diazepane-1-carboxylate (300 mg, 1.50 mmol), 2-chloro-5-(trifluoromethyl)pyrimidine (273 mg, 3.61 mmol) and K₂CO₃ (352 mg, 2.55 mmol) in ACN (5 mL) was stirred at 60° C. for 16 hours. The reaction was concentrated in vacuo, diluted with water (30 mL) and extracted with EtOAc (30 mL×3). The combined organic layer was washed with brine, dried over anhydrous Na₂SO₄, filtered and concentrated in vacuo. The residue was purified by silica gel chromatography (EtOAc/petroleum ether=10% to 20%) to give tert-butyl 4-(5-(trifluoromethyl)pyrimidin-2-yl)-1,4-diazepane-1-carboxylate (466 mg, 90% Yield) as a white solid. LCMS ESI m/z: 347 [M+H]⁺.

### Step 2

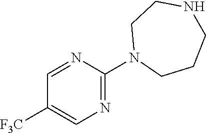 [SMILES: FC(F)(F)c1cnc(N2CCCNCC2)nc1]

### 1-(5-(Trifluoromethyl)pyrimidin-2-yl)-1,4-diazepane

To a solution of tert-butyl 4-(5-(trifluoromethyl)pyrimidin-2-yl)-1,4-diazepane-1-carboxylate (466 mg, 1.35 mmol)) in dioxane (1 mL) was added HCl/dioxane (4.0M in dioxane, 2 mL, 8.0 mmol). The mixture was stirred at rt for 1 hour. The solvent was removed in vacuo to give 1-(5-(trifluoromethyl)pyrimidin-2-yl)-1,4-diazepane (203 mg 61% yield) as a white solid. This was used without further purification. LCMS ESI m/z: 548 [M+H]⁺.

### Step 3

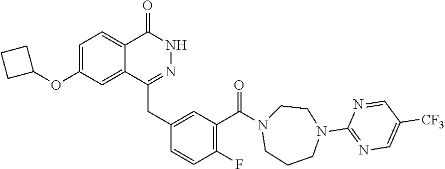 [SMILES: O=C(c1cc(Cc2n[nH]c(=O)c3ccc(OC4CCC4)cc23)ccc1F)N1CCCN(c2ncc(C(F)(F)F)cn2)CC1]

### 6-Cyclobutoxy-4-(4-fluoro-3-(4-((trifluoromethyl)pyrimidin-2-yl)-1,4-diazepane-1-carbonyl)benzyl)phthalazin-1(2f)-one

To a solution of 1-(5-(trifluoromethyl)pyrimidin-2-yl)-1,4-diazepane (74 mg, 0.26 mmol) and 5-((7-cyclobutoxy-4-oxo-3,4-dihydrophthalazin-1-yl)methyl)-2-fluorobenzoic acid (80 mg, 0.22 mmol) in DMF (1 mL) was added EDCI (62 mg, 0.32 mmol), HOBt (44 mg, 0.32 mmol) and DIPEA (140 mg, 1.1 mmol). The reaction mixture was stirred at rt for 2 hours. The reaction was purified by prep-HPLC to give 6-cyclobutoxy-4-(4-fluoro-3-(4-(5-(trifluoromethyl)pyrimidin-2-yl)-1,4-diazepane-1-carbonyl)benzyl)phthalazin-1(2H)-one (45 mg, 61% yield) as a white solid. LCMS ESI m/z 597.3 [M+H]⁺. ¹H NMR (400 MHz, DMSO-d₆) δ 12.44 (d, J=4.5 Hz, 1H), 8.81-8.46 (m, 2H), 8.16 (dd, J=8.8, 2.3 Hz, 1H), 7.38-7.02 (m, 5H), 4.87-4.76 (m, 1H), 4.25 (d, J=23.3 Hz, 2H), 3.94 (d, J=28.3 Hz, 1H), 3.90-3.77 (m, 4H), 3.65 (s, 1H), 3.43 (t, J=5.6 Hz, 1H), 3.22 (t, J=5.8 Hz, 1H), 2.38 (s, 2H), 2.04-1.43 (m, 6H).

### Synthesis of Example 99: 6-(3-(5-((7-Cyclobutoxy-4-oxo-3,4-dihydrophthalazin-1-yl)methyl)-2-fluorobenzoyl)-3,8-diazabicyclo[3.2.1]octan-8-yl)nicotinonitrile

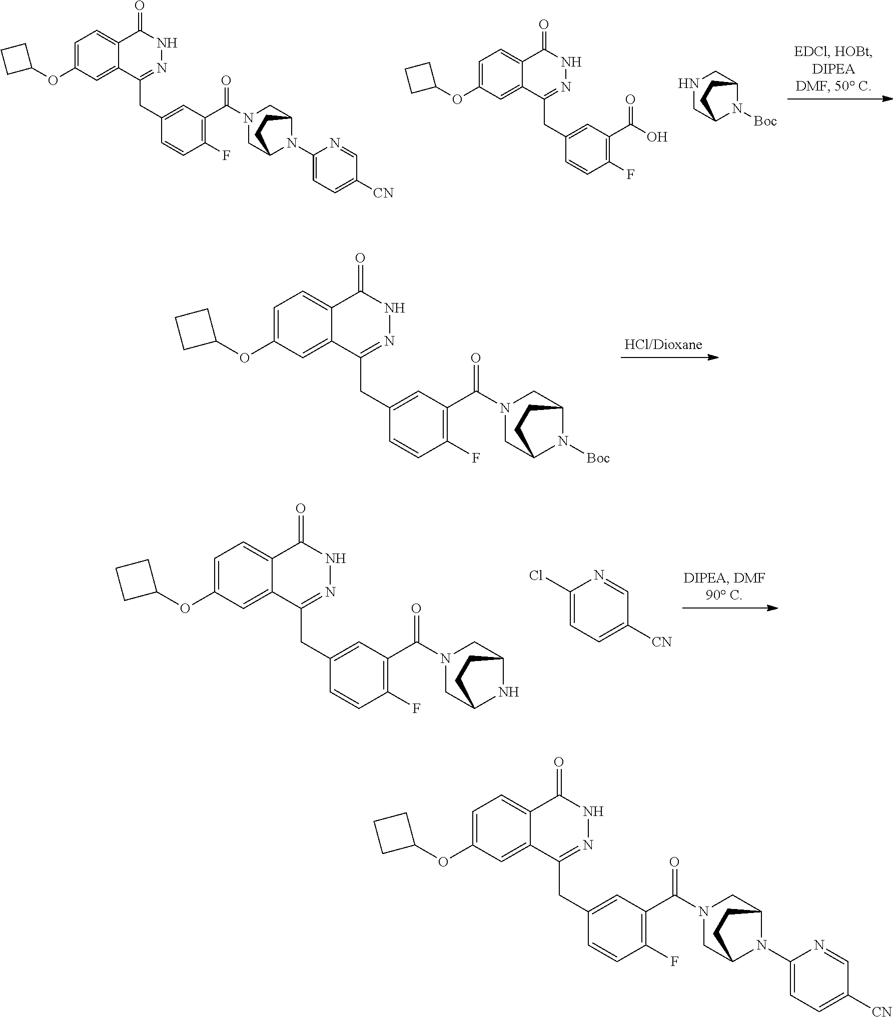

### Step 1

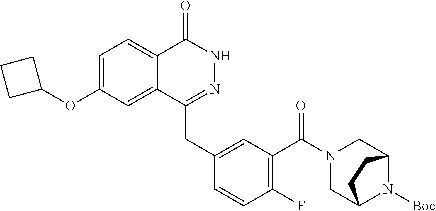

### tert-Butyl 3-(5-((7-cyclobutoxy-4-oxo-3,4-dihydrophthalazin-1-yl)methyl)-2-fluorobenzoyl)-3,8-diazabicyclo[3.2.1]octane-8-carboxylate

To a solution of 5-((7-cyclobutoxy-4-oxo-3,4-dihydrophthalazin-1-yl)methyl)-2-fluorobenzoic acid (30) mg, 0.81 mmol) and tert-butyl 3,8-diazabicyclo[3.2.1]octane-8-carboxylate (173 mg, 0.81 mmol) in DMF (3 mL) was added EDCI (165 mg, 1.22 mmol), HOBt (234 mg, 1.22 mmol) and DIPEA (316 mg, 2.44 mmol). The reaction mixture was stirred at rt for 16 hours. The mixture was diluted with H₂O (10 mL) and extracted with EtOAc (10 mL×3). The combined organic layer was washed with brine, dried over anhydrous Na₂SO₄, filtered and concentrated in vacuo. The residue was purified by silica gel chromatography (PE:EA=9:1 to 1:1) to give tert-butyl methyl(3-oxo-3-(4-(5-(trifluoromethyl)pyrimidin-2-yl)piperazin-1-yl)propyl)carbamate (426 mg, 0.75 mmol, 93% yield) as a white solid. LCMS ESI m/z: 563.1 [M+H]⁺.

### Step 2

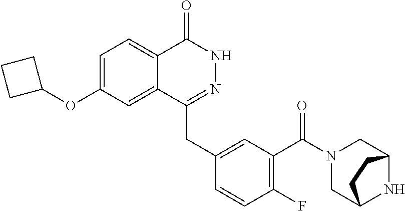

### 4-(3-(3,8-Diazabicyclo[3.2.1]octane-3-carbonyl)-4-fluorobenzyl)-6-cyclobutoxyphthalazin-1(2H)-one

To a solution of tert-butyl 3-(5-((7-cyclobutoxy-4-oxo-3,4-dihydrophthalazin-1-yl)methyl)-2-fluorobenzoyl)-3,8-diazabicyclo[3.2.1]octane-8-carboxylate (395 mg, 0.70 mmol) in dioxane (5 mL) was added HCl/Dioxane (4M, 5 mL) and stirred at 25° C. for 1 hour. The mixture was concentrated in vacuo to afford 4-(3-(3,8-diazabicyclo[3.2.1]octane-3-carbonyl)-4-fluorobenzyl)-6-cyclobutoxyphthalazin-1(2H)-one as a white solid (320 mg, 99% yield). This was used without further purification. LCMS ESI m/z: 463.1 [M+H]⁺.

### Step 3

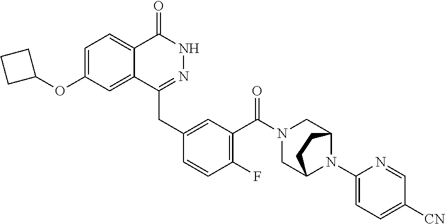

### 6-(3-(5-((7-Cyclobutoxy-4-oxo-3,4-dihydrophthalazin-1-yl)methyl)-2-fluorobenzoyl)-3,8-diazabicyclo[3.2.1]octan-8-yl)nicotinonitrile

To a solution of 4-(3-(3,8-diazabicyclo[3.2.1]octane-3-carbonyl)-4-fluorobenzyl)-6-cyclobutoxyphthalazin-1(2H)-one (80 mg, 0.17 mmol) and 6-chloronicotinonitrile (29 mg, 0.21 mmol) in DMF (1 mL) was added DIPEA (67 mg, 0.51 mmol). The reaction mixture was stirred at 90° C. for 16 hours. The reaction was purified by prep-HPLC to give 6-(3-(5-((7-cyclobutoxy-4-oxo-3,4-dihydrophthalazin-1-yl)methyl)-2-fluorobenzoyl)-3,8-diazabicyclo[3.2.1]octan-8-yl)nicotinonitrile (56.9 mg, 58% yield) as a white solid. LCMS ESI m/z: 565.0 [M+H]⁺. ¹H NMR (400 MHz, DMSO-d₆) δ 12.45 (s, 1H), 8.51 (d, J=2.1 Hz, 1H), 8.15 (d, J=8.8 Hz, 1H), 7.89 (dd, J=9.0, 2.3 Hz, 1H), 7.39 (dd, J=7.0, 4.6 Hz, 2H), 7.29 (dt, J=8.9, 4.5 Hz, 1H), 7.23 (t, J=8.8 Hz, 1H), 7.08 (s, 1H), 6.89 (d, J=9.0 Hz, 1H), 4.86-4.73 (m, 2H), 4.58 (s, 1H), 4.29 (s, 3H), 3.30-3.18 (m, 1H), 3.11 (d, J=12.3 Hz, 1H), 2.97 (d, J=12.6 Hz, 1H), 2.37 (d, J=5.4 Hz, 2H), 2.04-1.87 (m, 4H), 1.75 (t, J=8.6 Hz, 2H), 1.63 (d, J=8.6 Hz, 2H).

### Synthesis of Example 100: 6-Cyclobutoxy-4-(4-fluoro-3-(8-(5-(trifluoromethyl)pyrimidin-2-yl)-3,8-diazabicyclo[3.2.1]octane-3-carbonyl)benzyl)phthalazin-1(2H)-one

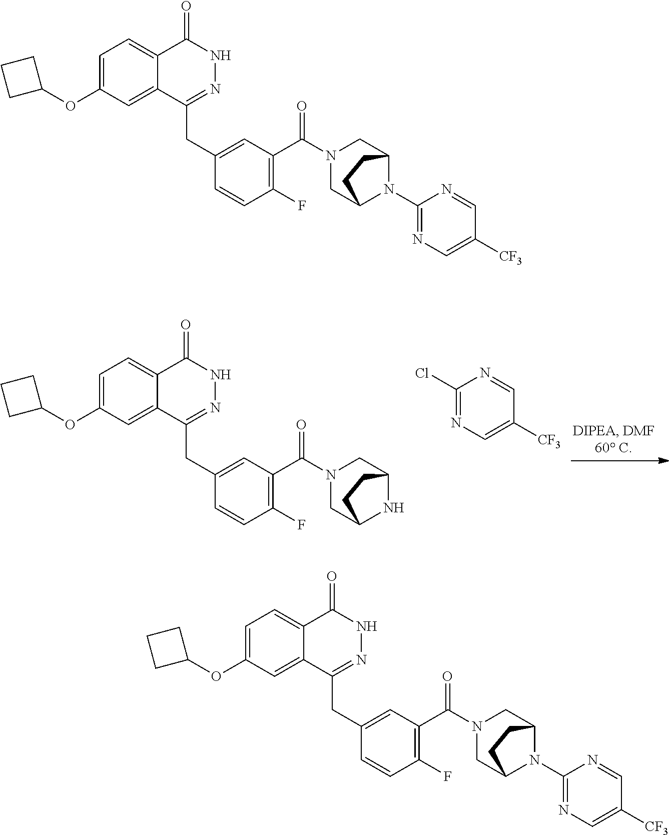

### Step 1

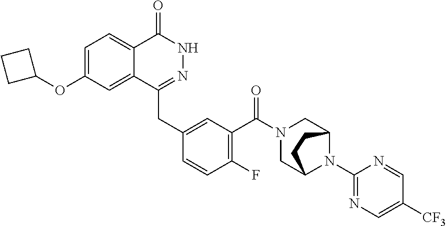

### 6-Cyclobutoxy-4-(4-fluoro-3-(8-(5-(trifluoromethyl)pyrimidin-2-yl)-3,8-diazabicyclo[3.2.1]octane-3-carbonyl)benzyl)phthalazin-1(2H)-one

To a solution of 4-(3-(3,8-diazabicyclo[3.2.1]octane-3-carbonyl)-4-fluorobenzyl)-6-cyclobutoxyphthalazin-1(2H)-one (100 mg, 0.22 mmol) and 2-chloro-5-(trifluoromethyl)pyrimidine (47 mg, 0.26 mmol) in DMF (3 mL) was added DIPEA (84 mg, 0.65 mmol). The reaction mixture was stirred at 60° C. for 3 hours. The mixture was purified by prep-HPLC to give 6-cyclobutoxy-4-(4-fluoro-3-(8-(5-(trifluoromethyl)pyrimidin-2-yl)-3,8-diazabicyclo[3.2.1]octane-3-carbonyl)benzyl) phthalazin-1(2H)-one (39 mg, 30% yield) as a white solid. LCMS ESI m/z: 609.2 [M+H]⁺. ¹H NMR (400 MHz, DMSO-d₆) δ 12.45 (s, 1H), 8.73 (s, 2H), 8.15 (d, J=8.8 Hz, 1H), 7.40 (dd, J=7.0, 4.6 Hz, 2H), 7.30 (dd, J=8.8, 2.3 Hz, 1H), 7.24 (t, J=8.9 Hz, 1H), 7.08 (s, 1H), 4.92-4.78 (m, 2H), 4.67 (d, J=6.1 Hz, 1H), 4.43-4.24 (m, 3H), 3.31-3.25 (m, 1H), 3.18 (d, J=14.8 Hz, 1H), 3.01 (d, J=12.4 Hz, 1H), 2.38 (d, J=6.6 Hz, 2H), 2.04-1.87 (m, 4H), 1.75 (t, J=9.6 Hz, 2H), 1.70-1.48 (m, 2H).

### Synthesis of Example 101: 6-Cyclobutoxy-4-(3-(4-(cyclopropanecarbonyl)-1,4-diazepane-1-carbonyl)-4-fluorobenzyl)phthalazin-1(2H)-one

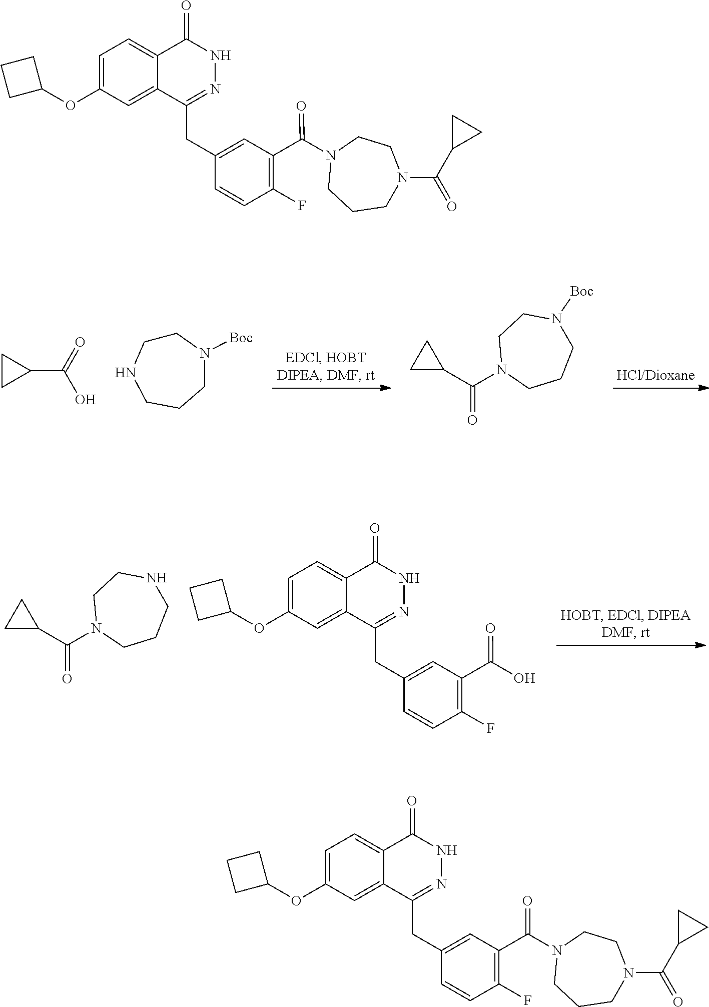

### Step 1

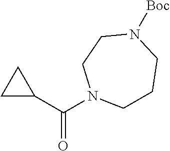 [SMILES: CC(C)(C)OC(=O)N1CCCN(C(=O)C2CC2)CC1]

### tert-Butyl 4-(cyclopropanecarbonyl)-1,4-diazepane-1-carboxylate

To a solution of tert-butyl 1,4-diazepane-1-carboxylate (300 mg, 1.50 mmol) and cyclopropanecarboxylic acid (155 mg, 1.80 mmol) in DMF (2 mL) was added EDCI (471 mg, 2.25 mmol). HOBt (304 mg, 2.25 mmol) and DIPEA (581 mg, 4.49 mmol). The resulting reaction mixture was stirred at rt for 16 hours, then the reaction was quenched with water (10 mL) and extracted with EtOAc (10 mL×3). The combined organic phase was washed with brine, dried over Na₂SO₄, filtered and concentrated under reduced pressure. The residue was purified by silica gel chromatography (Petroleum ether/EtOAc=4/1) to give tert-butyl 4-(cyclopropanecarbonyl)-1,4-diazepane-1-carboxylate (380 mg, 95% yield) as a colorless oil. LCMS EST m/z: 269.1 [M+H]⁺.

### Step 2

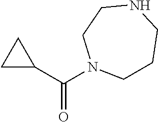 [SMILES: O=C(C1CC1)N1CCCNCC1]

### Cyclopropyl(1,4-diazepan-1-yl)methanone

To a solution of tert-butyl 4-(cyclopropanecarbonyl)-1,4-diazepane-1-carboxylate (380 mg, 1.42 mmol) in dioxane (5 mL) was added HCl/Dioxane (4M in dioxane, 5 mL) and stirred at 25° C. for 1 hour. The mixture was concentrated in vacuo to afford cyclopropyl(1,4-diazepan-1-yl)methanone as a white solid (238 mg, 82% yield). This was used without further purification. LCMS ESI m/z: 169.1 [M+H]⁺.

### Step 3

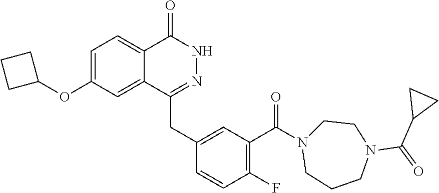 [SMILES: O=C(c1cc(Cc2n[nH]c(=O)c3ccc(OC4CCC4)cc23)ccc1F)N1CCCN(C(=O)C2CC2)CC1]

### 6-Cyclobutoxy-4-(3-(4-(cyclopropanecarbonyl)-1,4-diazepane-1-carbonyl)-4-fluorobenzyl)phthalazin-1(2H)-one

To a solution of cyclopropyl(1,4-diazepan-1-yl)methanone (50 mg, 0.30 mmol) and 5-((7-cyclobutoxy-4-oxo-3,4-dihydrophthalazin-1-yl)methyl)-2-fluorobenzoic acid (109 mg, 0.30 mmol) in DMF (2 mL) was added EDCI (85 mg, 0.45 mmol). HOBt (60 mg, 0.45 mmol) and DIPEA (115 mg, 0.90 mmol). The reaction mixture was stirred at rt for 16 hours. The mixture was poured into water (10 mL) and extracted with EtOAc (20 mL×3). The combined organic layer was washed with brine, dried over Na₂SO₄ and concentrated in vacuo. The residue was purified by prep-HPLC to give 6-cyclobutoxy-4-(3-(4-(cyclopropanecarbonyl)-1,4-diazepane-1-carbonyl)-4-fluorobenzyl)phthalazin-1(2H)-one (43 mg, 28% yield) as a white solid. LCMS ESI m/z: 519.1 [M+H]⁺. ¹H NMR (400 MHz, DMSO-d₆) δ 12.48-12.40 (1H, m), 8.15 (1H, d, J=8.8), 7.45-7.35 (1H, m), 7.33-7.15 (3H, m), 7.13-7.03 (1H, m), 4.83 (1H, dt, J=10.8, 7.0), 4.29 (2H, t, J=6.1), 3.81 (1H, t, J=10.9), 3.69 (1H, d, J=5.4), 3.65-3.55 (2H, m), 3.41 (2H, d, J=5.9), 3.27-3.15 (2H, m), 2.43-2.30 (2H, m), 2.05-1.95 (2H, m), 1.92-1.82 (1H, m), 1.82-1.57 (3H, m), 1.34 (1H, t, J=37.5), 0.59 (4H, dt, J=86.1, 5.5).

### Synthesis of Example 102: 6-Cyclobutoxy-4-(4-fluoro-3-(3-((5-(trifluoromethyl)pyrimidin-2-yl)amino)azetidine-1-carbonyl)benzyl)phthalazin-1(2H)-one

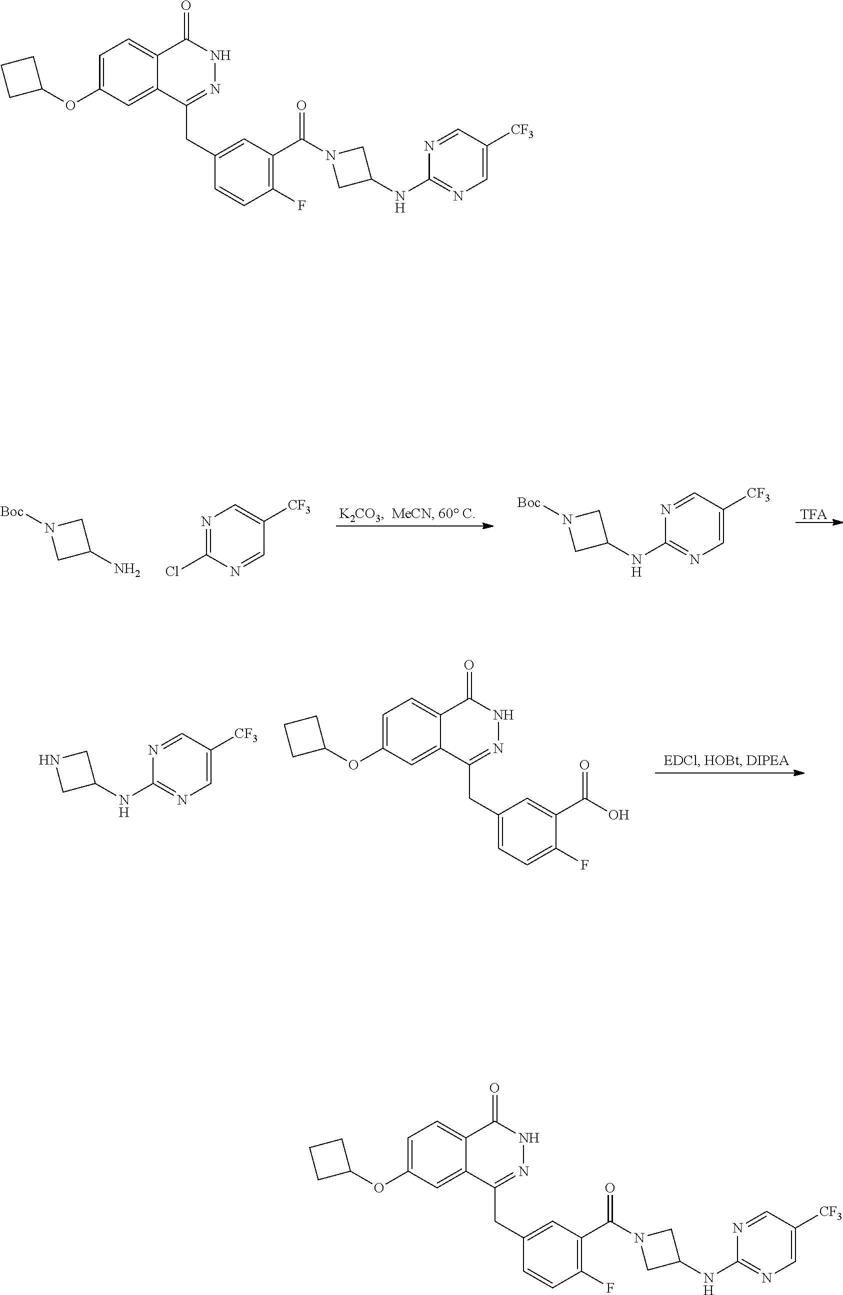

### Step 1

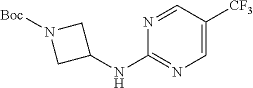 [SMILES: CC(C)(C)OC(=O)N1CC(Nc2ncc(C(F)(F)F)cn2)C1]

### tert-Butyl 3-((5-(trifluoromethyl)pyrimidin-2-yl)amino)azetidine-1-carboxylate

To a solution of 2-chloro-5-(trifluoromethyl)pyrimidine (300 mg, 1.64 mmol) and tert-butyl 3-aminoazetidine-1-carboxylate (283 mg, 1.64 mmol) in MeCN (3 mL) was added K₂CO₃ (681 mg, 4.93 mmol). The reaction mixture was stirred at 60° C. for 16 hours, then quenched with water (10 mL) and extracted with EtOAc (10 mL). The organic phase was washed with brine, dried over Na₂SO₄, filtered and concentrated under reduced pressure. The residue was purified by silica gel chromatography (Petroleum ether/EtOAc=5/1) to give tert-butyl 3-((5-(trifluoromethyl)pyrimidin-2-yl)amino)azetidine-1-carboxylate (520 mg, 99% yield) as a white solid. LCMS EST m/z: 319.1 [M+H]⁺.

### Step 2

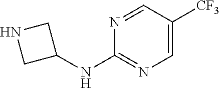 [SMILES: FC(F)(F)c1cnc(NC2CNC2)nc1]

### N-(Azetidin-3-yl)-5-(trifluoromethyl)pyrimidin-2-amine

To a solution of tert-butyl (1-(5-((7-cyclobutoxy-4-oxo-3,4-dihydrophthalazin-1-yl)methyl)-2-fluorobenzoyl)azetidin-3-yl)(methyl)carbamate (222 mg, 0.41 mmol) in DCM (10 mL) was added TFA (1 mL) and stirred at 25° C. for 2 hours. The reaction mixture was concentrated in vacuo to afford 6-cyclobutoxy-4-(4-fluoro-3-(3-(methylamino)azetidine-1-carbonyl)benzyl)phthalazin-1(2H)-one (346 mg, 97% yield) as a colorless oil. This was used without further purification. LCMS ESI m/z: 219.1 [M+H]⁺.

### Step 3

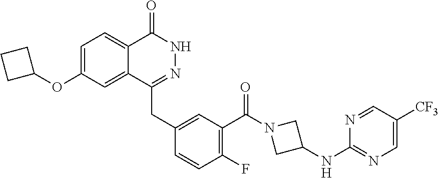 [SMILES: O=C(c1cc(Cc2n[nH]c(=O)c3ccc(OC4CCC4)cc23)ccc1F)N1CC(Nc2ncc(C(F)(F)F)cn2)C1]

### 6-Cyclobutoxy-4-(4-fluoro-3-(3-((5-(trifluoromethyl)pyrimidin-2-yl)amino)azetidine-1-carbonyl)benzyl)phthalazin-1(2H)-one

To a solution of N-(azetidin-3-yl)-5-(trifluoromethyl)pyrimidin-2-amine (50 mg, 0.23 mmol) and 5-((7-cyclobutoxy-4-oxo-3,4-dihydrophthalazin-1-yl)methyl)-2-fluorobenzoic acid (50 mg, 0.23 mmol) in DMF (2 mL) was added EDCI (66 mg, 0.34 mmol), HOBt (46 mg, 0.34 mmol) and DIPEA (89 mg, 0.69 mmol). The reaction mixture was stirred at rt for 16 h. The mixture was diluted with H₂O (10 mL) and extracted with EtOAc (10 mL×3). The combined organic layer was washed with brine, dried over anhydrous Na₂SO₄, filtered and concentrated in vacuo. The residue was purified by prep-HPLC to give 6-cyclobutoxy-4-(4-fluoro-3-(3-((5-(trifluoromethyl)pyrimidin-2-yl)amino) azetidine-1-carbonyl)benzyl)phthalazin-1(2H)-one (88.7 mg, 68% yield) as a white solid. LCMS ESI m/z: 569.2 [M+H]⁺. ¹H NMR (400 MHz, DMSO-d₆) δ 12.45 (1H, s), 8.71 (1H, d, J=6.1), 8.67 (1H, s), 8.14 (1H, d, J=8.8), 7.46 (2H, dd, J=12.0, 4.9), 7.33-7.17 (2H, m), 7.08 (1H, d, J=1.8), 4.81 (1H, dd, J=14.0, 7.1), 4.66 (1H, d, J=5.8), 4.41-4.17 (4H, m), 3.94 (2H, ddd, J=13.9, 9.4, 5.2), 2.41-2.26 (2H, m), 1.96 (2H, d, J=9.8), 1.82-1.53 (2H, m).

### Synthesis of Example 103: 6-Cyclobutoxy-4-(4-fluoro-3-(4-(5-(trifluormethyl)pyrimidin-2-yl)octahydropyrrolo[3,2-b]pyrrole-1-carbonyl)benzyl)phthalazin-1(2H)-one

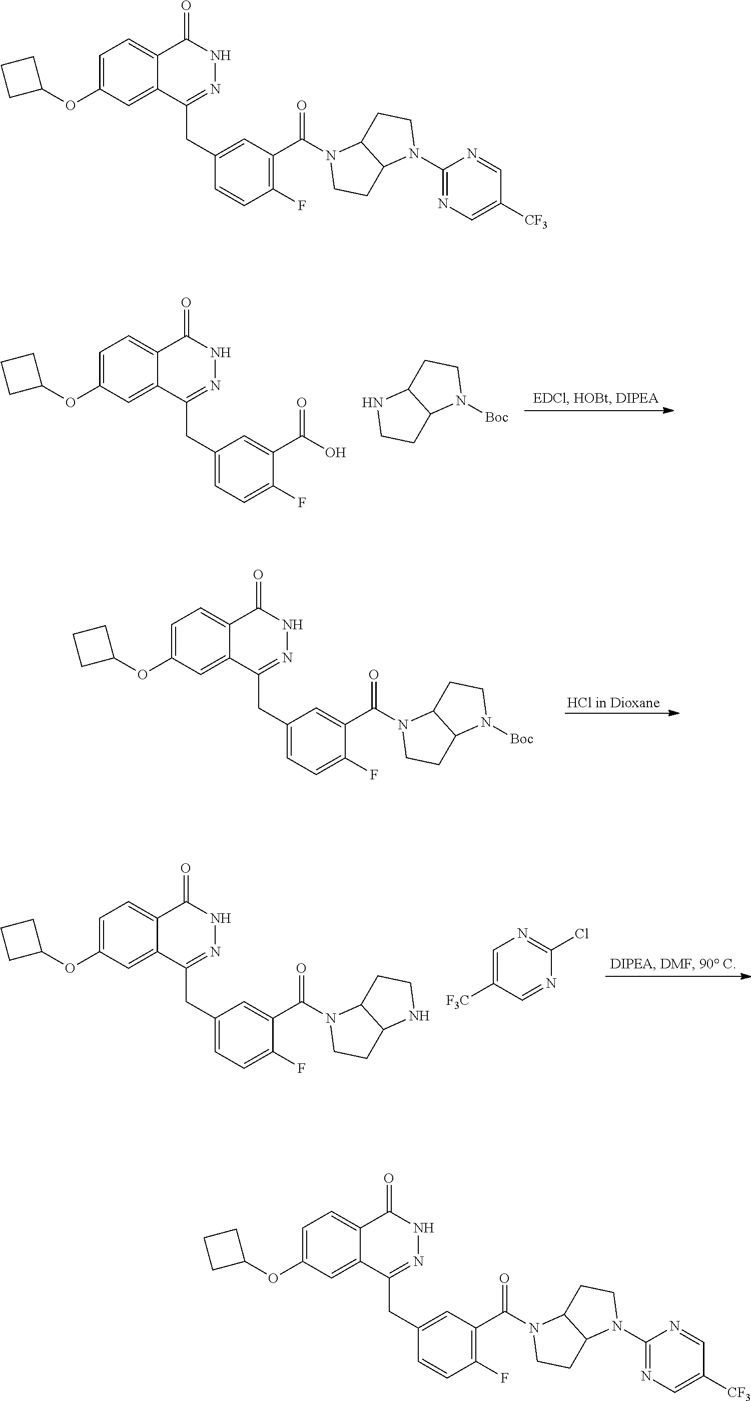

### Step 1

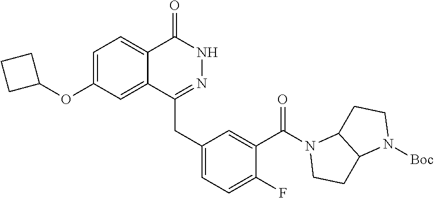 [SMILES: CC(C)(C)OC(=O)N1CCC2C1CCN2C(=O)c1cc(Cc2n[nH]c(=O)c3ccc(OC4CCC4)cc23)ccc1F]

### tert-Butyl 4-(5-((7-Cyclobutoxy-4-oxo-3,4-dihydrophthalazin-1-yl)methyl)-2-fluoro benzoyl)hexahydropyrrolo[3,2-b]pyrrole-1(2H)-carboxylate

To a solution of 5-((7-cyclobutoxy-4-oxo-3,4-dihydrophthalazin-1-yl)methyl)-2-fluorobenzoic acid (250 mag, 0.68 mmol) and tert-butyl hexahydropyrrolo[3,2-b]pyrrole-1(2H)-carboxylate (173 mg, 0.81 mmol) in DMF (2 mL) was added EDCI (195 mg, 1.02 mmol). HOBt (138 mg, 1.02 mmol) and DIPEA (439 mg, 3.40 mmol). The reaction mixture was stirred at rt for 16 hours. The mixture was diluted with H₂O (10 mL) and extracted with EtOAc (10 mL×3). The combined organic layer was washed with brine, dried over anhydrous Na₂SO₄, filtered and concentrated in vacuo. The residue was purified by silica gel chromatography (EtOAc/pet ether=40% to 50%) to give tert-butyl 4-(5-((7-cyclobutoxy-4-oxo-3,4-dihydrophthalazin-1-yl)methyl)-2-fluorobenzoyl)hexahydropyrrolo[3,2-b]pyrrole-1(2H)-carboxylate (360 mg 94% yield) as a white solid; LCMS ESI m/z 563.3 [M+H]⁺.

### Step 2

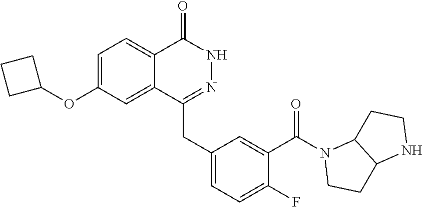 [SMILES: O=C(c1cc(Cc2n[nH]c(=O)c3ccc(OC4CCC4)cc23)ccc1F)N1CCC2NCCC21]

### 6-Cyclobutoxy-4-(4-fluoro-3-(octahydropyrrolo[3,2-b]pyrrole-1-carbonyl)benzyl)phthalazin-1(2H)-one

To a solution of tert-butyl 4-(5-((7-cyclobutoxy-4-oxo-3,4-dihydrophthalazin-1-yl)methyl)-2-fluorobenzoyl)hexahydropyrrolo[3,2-b]pyrrole-1(2H)-carboxylate (360 mg, 0.64 mmol) in dioxane (1 mL) was added HCl/dioxane (4.0 M, 2 mL, 8.00 mmol). The mixture was stirred at rt for 1 hour. The solution was concentrated in vacuo to give 6-cyclobutoxy-4-(4-fluoro-3-(octahydropyrrolo[3,2-b]pyrrole-1-carbonyl)benzyl) phthalazin-1(2H)-one (310 mg 97% yield) as a white solid. This was used without further purification. LCMS ESI m/z 463.1 [M+H]⁺.

### Step 3

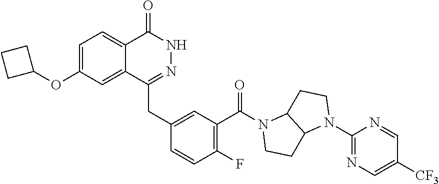 [SMILES: O=C(c1cc(Cc2n[nH]c(=O)c3ccc(OC4CCC4)cc23)ccc1F)N1CCC2C1CCN2c1ncc(C(F)(F)F)cn1]

### 6-Cyclobutoxy-4-(4-fluoro-3-(4-(5-(trifluoromethyl)pyrimidin-2-yl)octahydropyrrolo[3,2-b]pyrrole-1-carbonyl)benzyl)phthalazin-1(2H)-one

A solution of 6-cyclobutoxy-4-(4-fluoro-3-(octahydropyrrolo[3,2-b]pyrrole-1-carbonyl)benzyl)phthalazin-1(2H)-one (150 mg, 0.30 mmol), 2-chloro-5-(trifluoromethyl)pyrimidine (55 mg, 0.30 mmol) and DIPEA (155 mg, 1.20 mmol) in DMF (2 mL) was stirred at rt under Ar (g) for 12 hours, then purified by pre-HPLC to afford 6-cyclobutoxy-4-(4-fluoro-3-(4-(5-(trifluoromethyl)pyrimidin-2-yl)octahydropyrrolo[3,2-b]pyrrole-1-carbonyl)benzyl)phthalazin-1(2H)-one (56 mg 31% yield) as a colorless oil. LCMS ESI m/z 609.2 [M+H]⁺. ¹H NMR (400 MHz, DMSO-d6) δ 12.46 (s, 1H), 8.75 (s, 2H), 8.15 (m, 1H), 7.60-7.06 (m, 5H), 4.94-4.68 (m, 3H), 4.38-4.26 (m, 2H), 4.17-3.94 (m, 1H), 3.46 (m, 1H), 3.31-3.10 (m, 2H), 2.40-2.27 (m, 3f), 2.21-2.12 (m, 2H), 2.08-1.87 (m, 3H), 1.67 (m, 2H).

### Synthesis of Example 104: 6-((1-(5-((7-Cyclobutoxy-4-oxo-3,4-dihydrophthalazin-1-yl)methyl)-2-fluorobenzoyl) azetidin-3-yl)amino)nicotinonitrile

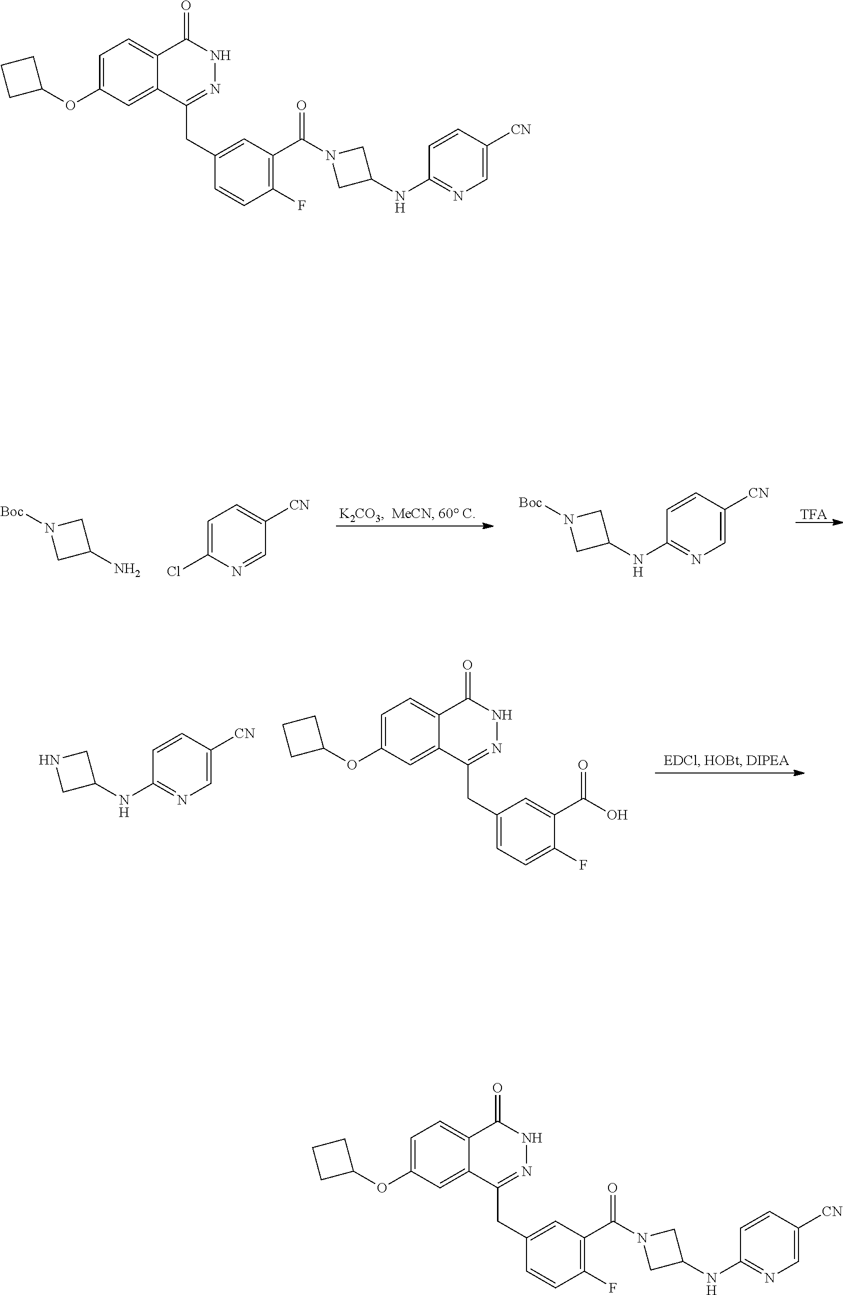

### Step 1

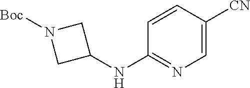 [SMILES: CC(C)(C)OC(=O)N1CC(Nc2ccc(C#N)cn2)C1]

### tert-Butyl 3-((5-Cyanopyridin-2-yl)amino)azetidine-1-carboxylate

A mixture of 6-chloropyridine-3-carbonitrile (300 mg, 2.17 mmol), tert-butyl 3-aminoazetidine-1-carboxylate (746 mg, 4.33 mmol) and K₂CO₃ (748 mg, 5.41 mmol) in MeCN (10 mL) was stirred under reflux for 16 hours. The reaction was concentrated in vacuo, diluted with water (30 mL) and extracted with EtOAc (20 mL×3). The combined organic layer was washed with brine, dried over anhydrous Na₂SO₄, filtered and concentrated in vacuo. The residue was purified by silica gel chromatography (PE/EA=5/1) to give tert-butyl 3-((5-cyanopyridin-2-yl)amino)azetidine-1-carboxylate (384 mg, 51% yield) as a white solid. LCMS ESI m/z: 525 [M+H]⁺.

### Step 2

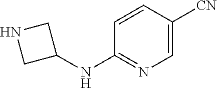 [SMILES: N#Cc1ccc(NC2CNC2)nc1]

### 6-(Azetidin-3-ylamino)nicotinonitrile

A solution of ten-butyl 3-((5-cyanopyridin-2-yl)amino)azetidine-1-carboxylate (384 mg, 1.12 mmol) in DCM (5 mL) and TFA (0.5 mL) was stirred at rt for 1 hour. The solvent was removed in vacuo to give 6-(azetidin-3-ylamino)nicotinonitrile (322 mg, 100% yield) as a pale yellow oil. LCMS ESI m/z: 175.1 [M+H]⁺. This was used without further purification.

### Step 3

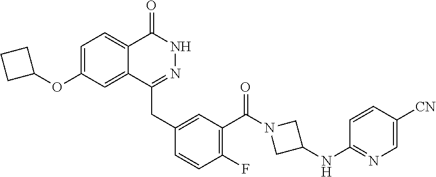 [SMILES: N#Cc1ccc(NC2CN(C(=O)c3cc(Cc4n[nH]c(=O)c5ccc(OC6CCC6)cc45)ccc3F)C2)nc1]

### 6-((1-(5-((7-cyclobutoxy-4-oxo-3,4-dihydrophthalazin-1-yl)methyl)-2-fluorobenzoyl) azetidin-3-yl)amino)nicotinonitrile

To a solution of 5-((7-cyclobutoxy-4-oxo-3,4-dihydrophthalazin-1-yl)methyl)-2-fluorobenzoic acid (80 mg, 0.22 mmol) and 6-(azetidin-3-ylamino)nicotinonitrile (270 mg, 0.44 mmol) in DMF (2 mL) was added EDCI (62 mg, 0.33 mmol), HOBt (44 mg, 0.33 mmol) and DIPEA (140 mg, 1.09 mmol). The reaction mixture was stirred at rt for 1 hour. The reaction was purified by prep-HPLC to give 6-((1-(5-((7-cyclobutoxy-4-oxo-3,4-dihydrophthalazin-1-yl)methyl)-2-fluorobenzoyl)azetidin-3-yl)amino)nicotinonitrile (54 mg, 47% yield) as a white solid. LCMS ESI m/z: 525.0 [M+H]⁺. ¹H-NMR (400 MHz, DMSO-d₆) δ 12.45 (s, 1H), 8.40 (d, J=2.0 Hz, 1H), 8.23 (d, J=6.0 Hz, 1H), 8.14 (d, J=8.8 Hz, 1H), 7.75 (dd, J=8.8, 2.2 Hz, 1H), 7.51-7.39 (m, 2H), 7.30-7.18 (m, 2H), 7.07 (d, J=2.3 Hz, 1H), 6.56 (d, J=8.8 Hz, 1H), 4.81 (p, J=7.2 Hz, 1H), 4.62 (s, 1H), 4.40-4.20 (m, 4H), 3.89 (dd, J=10.2, 5.1 Hz, 1H), 3.79 (dd, J=8.9, 5.0 Hz, 1H), 2.40-2.28 (m, 2H), 1.97 (dd, =10.0, 7.8 Hz, 2H), 1.80-1.56 (m, 2H).

### Synthesis of Example 105: 5-((1-(5-((7-cyclobutoxy-4-oxo-3,4-dihydrophthalazin-1-yl)methyl)-2-fluorobenzoyl)azetidin-3-yl)(methyl)amino)nicotinonitrile

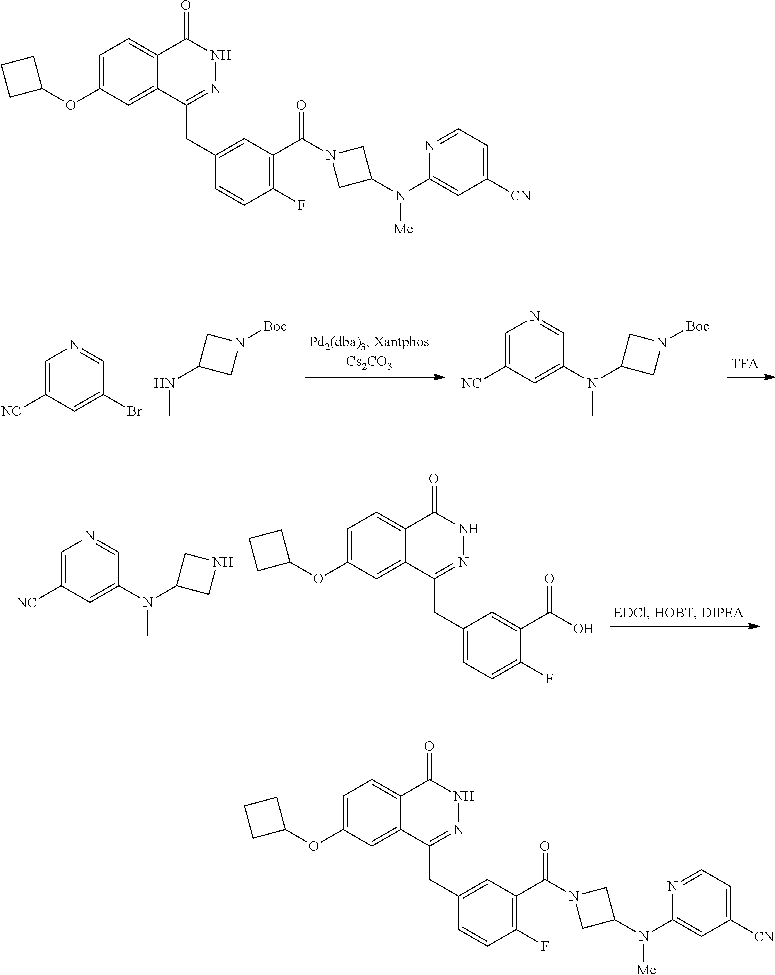

### Step 1

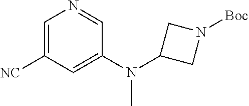 [SMILES: CN(c1cncc(C#N)c1)C1CN(C(=O)OC(C)(C)C)C1]

### tert-Butyl 3-((5-cyanopyridin-3-yl)(methyl)amino)azetidine-1-carboxylate

A mixture of 5-bromonicotinonitrile (300 mg, 1.64 mmol), tert-butyl 3-(methylamino)azetidine-1-carboxylate (458 mg, 2.46 mmol), Pd₂(dba)₃ (150 mg, 0.16 mmol), Xantphos (190 mg, 0.33 mmol) and t-BuONa (236 mg, 2.46 mmol) in toluene (5 mL) was stirred at 115° C. for 3 hours. The mixture was cooled to rt, diluted with H₂O (5 mL), and extracted with EtOAc (10 mL×3). The combined organic layer was washed by brine, dried over Na₂SO₄, filtered and concentrated in vacuo. The residue was purified by silica gel chromatography (EtOAc/pet ether=20 to 35%) to give tert-butyl 3-((5-cyanopyridin-3-yl)(methyl)amino)azetidine-1-carboxylate as a yellow oil (244 mg, 51% yield). LCMS ESI m/z 289 [M+H]⁺.

### Step 2

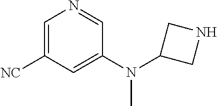

### 5-(Azetidin-3-yl(methyl)amino)nicotinonitrile

A solution of tert-butyl 3-((5-cyanopyridin-3-yl)(methyl)amino)azetidine-1-carboxylate (244 mg, 0.85 mmol) in DCM (3 mL) and TFA (0.3 mL) was stirred at rt for 1 hour. The reaction was concentrated under vacuum to afford 5-(azetidin-3-yl(methyl)amino)nicotinonitrile as a yellow oil (155 mg, 97% yield). This was used without further purification. LCMS ESI m, 189 [M+H]⁺.

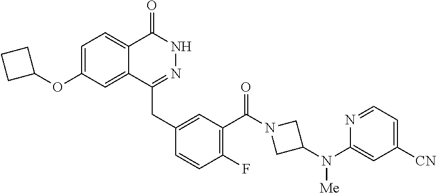 [SMILES: CN(c1cc(C#N)ccn1)C1CN(C(=O)c2cc(Cc3n[nH]c(=O)c4ccc(OC5CCC5)cc34)ccc2F)C1]

### 5-((1-(5-((7-Cyclobutoxy-4-oxo-3,4-dihydrophthalazin-1-yl)methyl)-2-fluorobenzoyl)azetidin-3-yl)(methyl)amino)nicotinonitrile

To a solution of 5-((7-cyclobutoxy-4-oxo-3,4-dihydrophthalazin-1-yl)methyl)-2-fluorobenzoic acid (60 mg, 0.16 mmol) and 5-(azetidin-3-yl(methyl)amino)nicotinonitrile (67 mg, 0.36 mmol) in DMF (2 mL) was added EDCI (47 mg, 0.24 mmol), HOBt (33 mg, 0.24 mmol) and DIPEA (63 mg, 0.49 mmol). The reaction mixture was stirred at rt for 24 hours, then purified by prep-HPLC to give 5-((1-(5-((7-cyclobutoxy-4-oxo-3,4-dihydrophthalazin-1-yl)methyl)-2-fluorobenzoyl)azetidin-3-yl)(methyl)amino) nicotinonitrile as a white solid (36 mg, 41% yield). LCMS ESI m/z 539 [M+H]⁺. ¹H-NMR (400 MHz, DMSO-d₆) δ 12.47 (s, 1H), 8.42 (d, J=3.0 Hz, 1H), 8.32 (d, J=1.5 Hz, 1H), 8.14 (d, J=8.8 Hz, 1H), 7.62 (dd, J=2.9, 1.7 Hz, 1H), 7.51 (dd, J=6.5, 2.1 Hz, 1H), 7.48-7.43 (m, 1H), 7.31-7.21 (m, 2H), 7.10 (d, J=2.3 Hz, 1H), 4.89-4.72 (m, 2H), 4.49-4.19 (m, 4H), 4.05-3.98 (m, 2H), 2.98 (s, 3H), 2.43-2.30 (m, 2H), 2.06-1.90 (m, 2H), 1.83-1.56 (m, 2H).

### Synthesis of Example 106: 2-(1-(5-((7-Cyclobutoxy-4-oxo-3,4-dihydrophthalazin-1-yl)methyl)-2-fluorobenzoyl)azetidin-3-ylamino)isonicotinonitrile

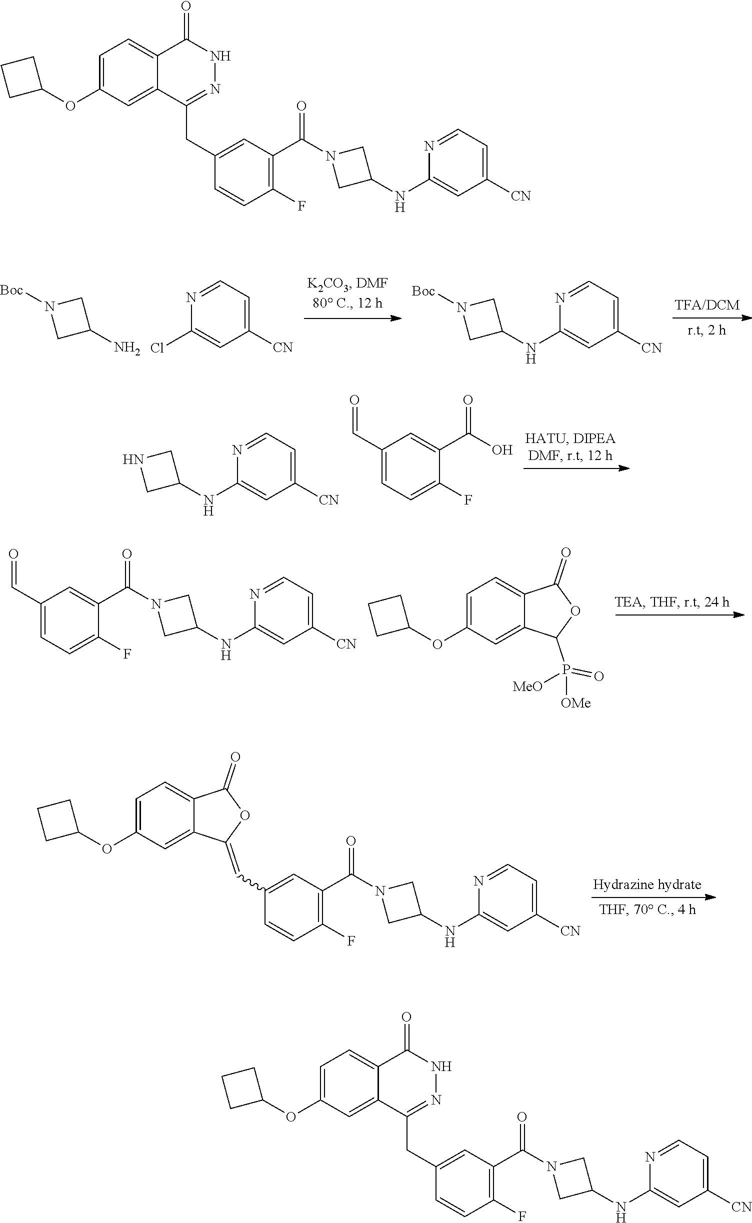

### Step 1

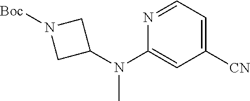 [SMILES: CN(c1cc(C#N)ccn1)C1CN(C(=O)OC(C)(C)C)C1]

### tert-Butyl 3-(4-cyanopyridin-2-ylamino)azetidine-1-carboxylate

tert-butyl 3-aminoazetidine-1-carboxylate (2.1 g, 12.19 mmol), 2-chloropyridine-4-carbonitrile (2.53 g, 18.29 mmol), potassium carbonate (3.37 g, 24.39 mmol, 1.47 mL) and DMF (20 mL) were added to a 100 mL bottled flask. The resultant mixture was stirred at 80° C. for 12 hr. The mixture was diluted with H₂O (20 mL) and extracted with EA (40 mL×2). The combined organic phases were washed with brine (30 mL), dried over Na₂SO₄ and evaporated, and the residue was purified by silica gel chromatography to give tert-butyl 3-(4-cyanopyridin-2-ylamino)azetidine-1-carboxylate (360 mg, 10.8% yield) as a yellow solid. LCMS ESI m/z: 275.1 [M+H]⁺.

### Step 2

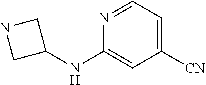 [SMILES: N#Cc1ccnc(NC2CNC2)c1]

### 2-(Azetidin-3-ylamino)isonicotinonitrile

tert-Butyl 3-[(4-cyano-2-pyridyl)amino]azetidine-1-carboxylate (360 mg, 1.31 mmol). TFA (2.96 g, 25.96 mmol, 2 mL) and DCM (4 mL) were added to a 50 mL bottled flask. The resultant mixture was stirred at rt for 2 hr. The solvent removed in vacuum to give 2-(azetidin-3-ylamino)pyridine-4-carbonitrile (600 mg, crude), which was used to next step without further purification. LCMS ESI m/z: 175.2 [M+H]⁺.

### Step 3

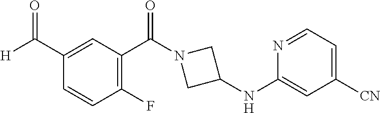 [SMILES: N#Cc1ccnc(NC2CN(C(=O)c3cc(C=O)ccc3F)C2)c1]

### 2-(1-(2-Fluoro-5-formylbenzoyl)azetidin-3-ylamino)isonicotinonitrile

2-(azetidin-3-ylamino)pyridine-4-carbonitrile (540.03 mg, 1.87 mmol) was added to a solution of 2-fluoro-5-formyl-benzoic acid (210 mg, 1.25 mmol), HATU (716.19 mg, 1.88 mmol). DIPEA (645.75 mg, 5.00 mmol, 870.28 μLL) and DMF (10 mL). The resultant mixture was stirred at rt for 12 hr. The mixture was diluted with H₂O (10 mL) and extracted with EtOAc (20 mL×2). The combined organic phases were washed with brine (20 mL), dried over Na₂SO₄ and concentrated. The residue was purified by Prep-HPLC to give 2-(1-(2-fluoro-5-formylbenzoyl)azetidin-3-ylamino)isonicotinonitrile (350 mg, 86% yield) as a yellow solid. LCMS EST m/z: 325.1 [M+H]⁺.

### Step 4

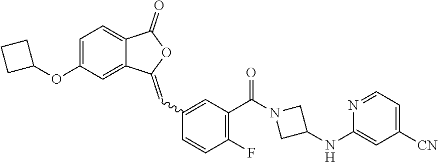 [SMILES: N#Cc1ccnc(NC2CN(C(=O)c3cc(C=C4OC(=O)c5ccc(OC6CCC6)cc54)ccc3F)C2)c1]

### 2-(1-(5-((6-Cyclobutoxy-3-oxoisobenzofuran-1(3H)-ylidene)methyl)-2-fluorobenzoyl)azetidin-3-ylamino)isonicotinonitrile

To a mixture of 5-(cyclobutoxy)-3-dimethoxyphosphoryl-3H-isobenzofuran-1-one (70 mg, 224.18 μmol) in THF (10 mL) was added 2-[[1-(2-fluoro-5-formyl-benzoyl)azetidin-3-yl]amino]pyridine-4-carbonitrile (109.05 mg, 336.26 μmol) and ET₃N (68.05 mg, 672.53 μmol, 93.74 μL). The resulting mixture was stirred at rt for 24 hr. The mixture was quenched with sat. NaHSO₃ solution (5 mL), and filtered. The filter cake was washed with H₂O (10 mL) and dried under reduced pressure to give 2-(1-(5-((6-cyclobutoxy-3-oxoisobenzofuran-1(3H)-ylidene)methyl)-2-fluorobenzoyl)azetidin-3-ylamino)isonicotinonitrile (70 mg, crude) as a yellow solid. LCMS ESI m/z: 510.7 [M+H]⁺.

### Step 5

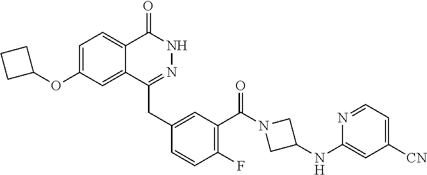 [SMILES: N#Cc1ccnc(NC2CN(C(=O)c3cc(Cc4n[nH]c(=O)c5ccc(OC6CCC6)cc45)ccc3F)C2)c1]

### 2-(1-(5-((7-Cyclobutoxy-4-oxo-3,4-dihydrophthalazin-1-yl)methyl)-2-fluorobenzoyl)azetidin-3-ylamino)isonicotinonitrile

To a mixture of 2-[[1-[5-[(E)-[6-(cyclobutoxy)-3-oxo-isobenzofuran-1-ylidene]methyl]-2-fluoro-benzoyl]azetidin-3-yl]amino]pyridine-4-carbonitrile (70 mg, 137.12 μmol) in THF (5 mL) was added N₂H₄·H₂O (8.58 mg, 137.12 μmol, 8.35 μL). The resulting mixture was stirred at 70° C. for 4 hr. The mixture was concentrated under reduced pressure to give the crude product, which was purified by Pre-HPLC to afford 2-[[1-[5-[[7-(cyclobutoxy)-4-oxo-3H-phthalazin-1-yl]methyl]-2-fluoro-benzoyl]azetidin-3-yl]amino]pyridine-4-carbonitrile (17.2 mg, 23.9% yield) as a white solid. LCMS ESI m/z: 525.2 [M+H]⁺. ¹H NMR (400 MHz, DMSO-D6) δ 12.44 (s, 1H), 8.15 (dd, J=11.5, 7.0 Hz, 2H), 7.78 (d, J=6.0 Hz, 1H), 7.49-7.41 (m, 2H), 7.26 (ddd, J=18.4, 9.3, 5.4 Hz, 2H), 7.08 (d, J=2.3 Hz, 1H), 6.89 (dd, J=5.2, 1.3 Hz, 1H), 6.82 (s, 1H), 4.85-4.78 (m, 1H), 4.59-4.52 (m, 1H), 4.37-4.23 (m, 4H), 3.89 (dd, J=10.2, 5.1 Hz, 1H), 3.76 (dd, J=8.8, 5.0 Hz, 1H), 2.40-2.31 (m, 2H), 1.97 (dd, J=9.7, 5.0 Hz, 2H), 1.79-1.60 (m, 2H).

### Synthesis of Example 107: 6-Cyclobutoxy-4-(3-(8-(cyclopropanecarbonyl)-3,8-diazabicyclo[3.2.1]octane-3-carbonyl)-4-fluorobenzyl)phthalazin-1(2H)-one

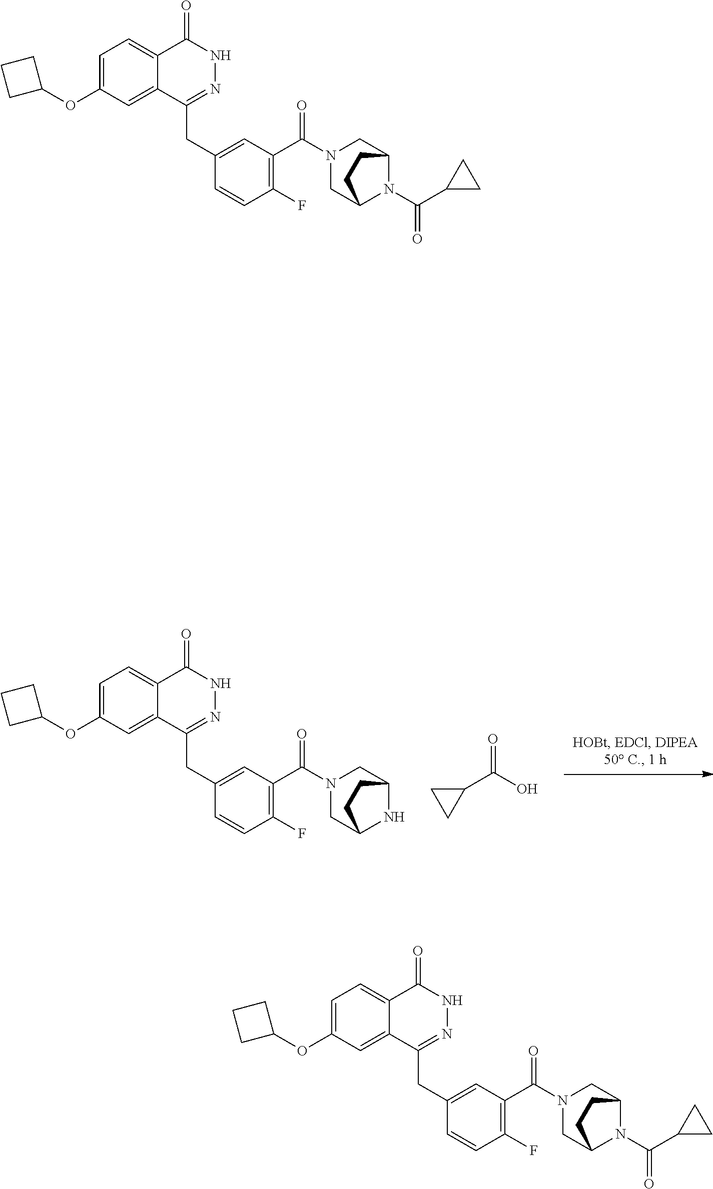

### Step 1

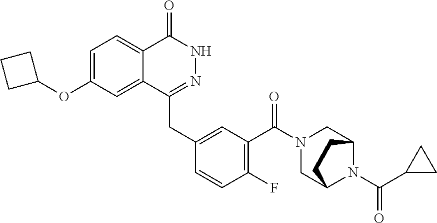

### 6-Cyclobutoxy-4-(3-(8-(cyclopropanecarbonyl)-3,8-diazabicyclo[3.2.1]octane-3-carbonyl)-4-fluorobenzyl)phthalazin-1(2H)-one

To a solution of 4-(3-(3,8-diazabicyclo[3.2.1]octane-3-carbonyl)-4-fluorobenzyl)-6-cyclobutoxyphthalazin-1(2H)-one (80 mg, 0.17 mmol) and cyclopropanecarboxylic acid (22 mg, 0.26 mmol) in DMF (2 mL) was added EDCI (50 mg, 0.26 mmol), HOBt (35 mg, 0.26 mmol) and DIPEA (67 mg, 0.51 mmol). The reaction mixture was stirred at 50° C. for 1 hour. The reaction was purified by prep-HPLC to give 6-cyclobutoxy-4-(3-(8-(cyclopropanecarbonyl)-3,8-diazabicyclo[3.2.1]octane-3-carbonyl)-4-fluorobenzyl) phthalazin-1(2H)-one (35 mg, 38% yield) as a white solid. LCMS ESI m/z: 531 [M+H]⁺. ¹H NMR (400 MHz, DMSO-d₆) δ 12.45 (s, 1H), 8.18-8.10 (m, 1H), 7.46-7.34 (m, 2H), 7.32-7.28 (m, 1H), 7.26-7.19 (m, 1H), 7.13-7.03 (m, 1H), 4.96-4.64 (m, 2H), 4.59-4.46 (m, 1H), 4.35-4.26 (m, 3H), 3.22-3.04 (m, 2H), 3.00-2.81 (m, 1H), 2.39 (s, 2H), 2.09-1.90 (m, 4H), 1.84-1.74 (m, 2H), 1.71-1.62 (m, 2H), 0.82-0.60 (m, 5H).

### Synthesis of Example 108: 2-((1-(5-((7-cyclobutoxy-4-oxo-3,4-dihydrophthalazin-1-yl)methyl)-2-fluorobenzoyl) azetidin-3-yl)(methyl)amino)-N-methylpyrimidine-4-carboxamide

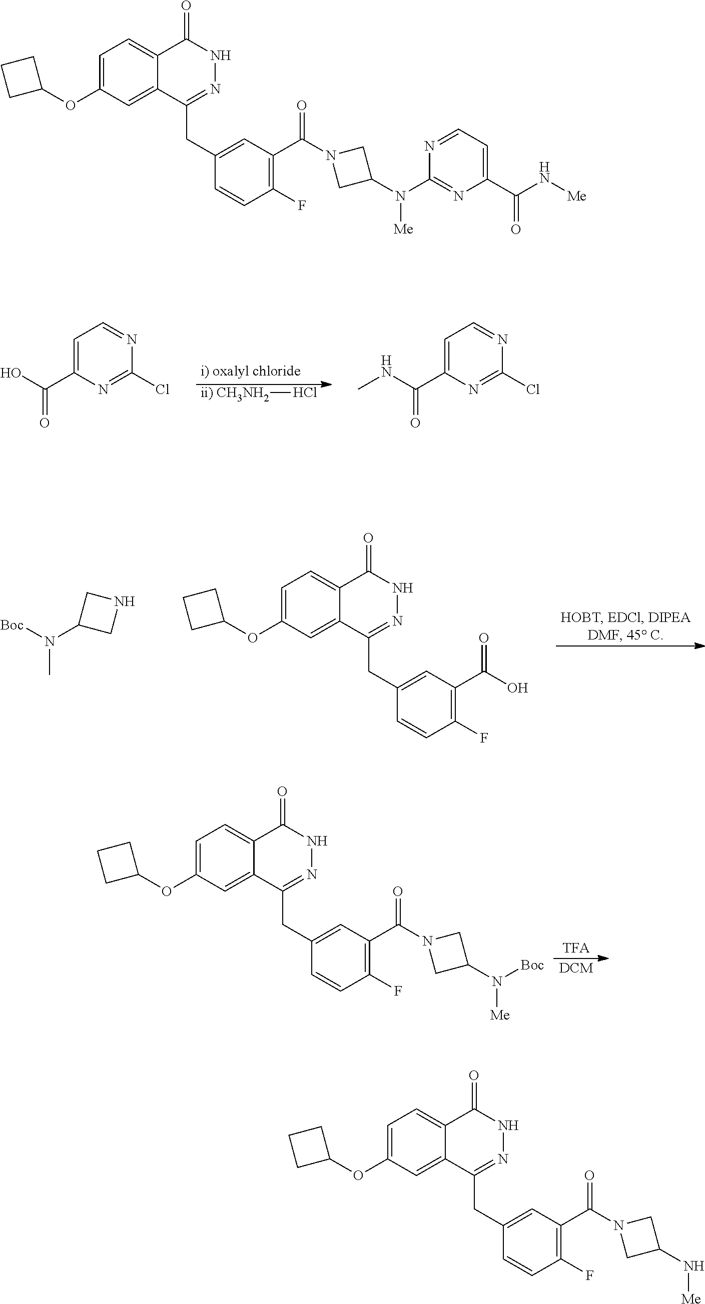

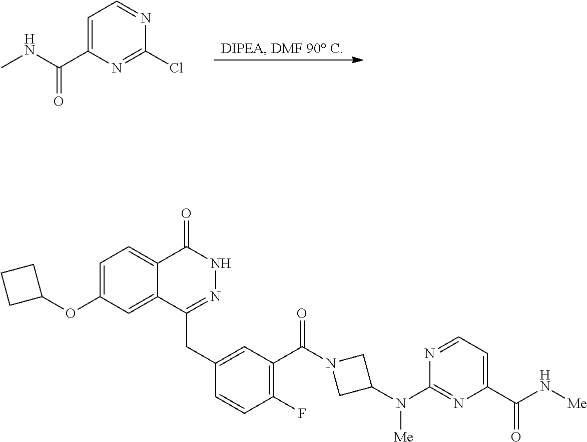 [SMILES: CNC(=O)c1ccnc(Cl)n1 ; "DIPEA, DMF 90° C." ; CNC(=O)c1ccnc(N(C)C2CN(C(=O)c3cc(Cc4n[nH]c(=O)c5ccc(OC6CCC6)cc45)ccc3F)C2)n1]

### Step 1

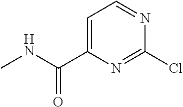

A solution of 2-chloropyrimidine-4-carboxylic acid (500 mg, 3.15 mmol). DMF (23 mg, 0.32 mmol) in DCM (10 mL) was added oxalyl dichloride (400 mg, 3.15 mmol) dropwise at 0° C. The reaction was stirred at rt for 1 hour, then concentrated in vacuo. The residue was dissolved in DCM (10 in L), then added a solution of methanamine hydrochloride (426 mg, 6.30 mmol) and DIPEA (1.22 g, 9.46 mmol) in DCM (10 mL). The mixture was stirred at rt for 1 hour, then diluted with H₂O (30 mL) and extracted with DCM (30 mL×3). The combined organic layer was dried over anhydrous Na₂SO₄, filtered and concentrated in vacuo. The residue was purified by silica gel chromatography (EtOAc/petroleum ether=0% to 30%) to give methyl 2,4-difluoro-5-vinyl-benzoate (310 mg, 57% yield) as a white solid. LCMS ESI m/z: 172 [M+H]⁺.

### Step 2

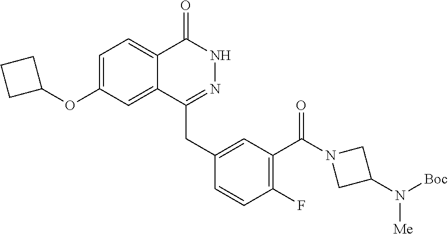 [SMILES: CN(C(=O)OC(C)(C)C)C1CN(C(=O)c2cc(Cc3n[nH]c(=O)c4ccc(OC5CCC5)cc34)ccc2F)C1]

### tert-Butyl (1-(5-((7-cyclobutoxy-4-oxo-3,4-dihydrophthalazin-1-yl)methyl)-2-fluorobenzoyl)azetidin-3-yl)(methyl)carbamate

To a solution of 5-((7-cyclobutoxy-4-oxo-3,4-dihydrophthalazin-1-yl)methyl)-2-fluorobenzoic acid (120 mg, 0.33 mmol) and tert-butyl azetidin-3-yl(methyl)carbamate (73 mg, 0.39 mmol) in DMF (2 mL) was added EDCI (62 mg, 0.33 mmol), HOBt (66 mg, 0.49 mmol) and DIPEA (126 mg, 0.98 mmol). The reaction mixture was stirred at 45° C. for 1 hour. The mixture was diluted with H₂O (20 mL) and extracted with EtOAc (20 mL×3). The combined organic layer was washed with brine, dried over anhydrous Na₂SO₄, filtered and concentrated in vacuo. The residue was purified by silica gel chromatography (EtOAc/pet ether=0% to 50%) to give tert-butyl (1-(5-((7-cyclobutoxy-4-oxo-3,4-dihydrophthalazin-1-yl)methyl)-2-fluorobenzoyl)azetidin-3-yl)(methyl)carbamate (100 mg, 57/o yield) as a white solid. LCMS ESI m/z: 537 [M+H]⁺.

### Step 3

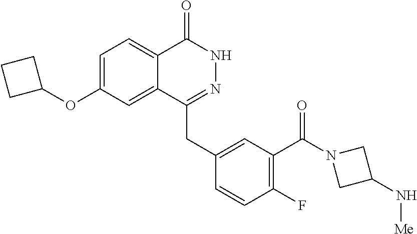 [SMILES: CNC1CN(C(=O)c2cc(Cc3n[nH]c(=O)c4ccc(OC5CCC5)cc34)ccc2F)C1]

### 6-Cyclobutoxy-4-(4-fluoro-3-(3-(methylamino)azetidine-1-carbonyl) benzyl)phthalazin-1(2H)-one

A solution of tert-butyl (1-(5-((7-cyclobutoxy-4-oxo-3,4-dihydrophthalazin-1-yl)methyl)-2-fluorobenzoyl)azetidin-3-yl)(methyl)carbamate (100 mg, 0.19 mmol) in DCM (5 mL) and TFA (0.5 mL) was stirred at rt for 1 hour. The solvent was removed in vacuo to give 6-cyclobutoxy-4-(4-fluoro-3-(3-(methylamino)azetidine-1-carbonyl)benzyl)phthalazin-1(2H)-one (80 mg, 78% yield) as a yellow oil. This was used without further purification. LCMS ESI m/z: 437 [M+H]⁺.

### Step 4

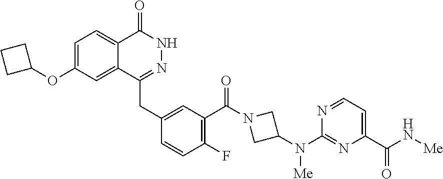 [SMILES: CNC(=O)c1ccnc(N(C)C2CN(C(=O)c3cc(Cc4n[nH]c(=O)c5ccc(OC6CCC6)cc45)ccc3F)C2)n1]

### 2-((1-(5-(7-Cyclobutoxy-4-oxo-3,4-dihydrophthalazin-1-yl)methyl)-2-fluorobenzoyl) azetidin-3-yl)(methyl)amino)-N-methylpyrimidine-4-carboxamide

A solution of 6-cyclobutoxy-4-(4-fluoro-3-(3-(methylamino)azetidine-1-carbonyl)benzyl)phthalazin-1(2H)-one (60 mg, 0.14 mmol), 2-chloro-N-methylpyrimidine-4-carboxamide (71 mg, 0.41 mmol) and DIPEA (53 mg, 0.41 mmol) in DMF (1 mL) was stirred at 90° C. for 2 hours. The mixture was concentrated in vacuo and the residue was purified by prep-HPLC to give 2-((1-(5-((7-cyclobutoxy-4-oxo-3,4-dihydrophthalazin-1-yl)methyl)-2-fluorobenzoyl)azetidin-3-yl)(methyl)amino)-N-methylpyrimidine-4-carboxamide (36 mg, 45% yield) as a white solid. LCMS ESI m/z: 572 [M+H]⁺. ¹H-NMR (400 MHz, DMSO-d₆) δ 12.47 (s, 1H), 8.76-8.70 (m, 1H), 8.58 (d, J=4.8 Hz, 1H), 8.15 (d, J=8.8 Hz, 1H), 7.54-7.50 (m, 1H), 7.48-7.43 (m, 1H), 7.30-7.23 (m, 2H), 7.16 (d, J=4.8 Hz, 1H), 7.12-7.10 (m, 1H), 5.74 (s, 1H), 4.86-4.78 (m, 1H), 4.37-4.30 (m, 3H), 4.23-4.14 (m, 3H), 3.21 (s, 3H), 2.81 (d, J=4.8 Hz, 3H), 2.39-2.32 (m, 2H), 2.02-1.92 (m, 2H), 1.78-1.59 (m, 2H).

### Synthesis of Example 109: 6-Cyclobutoxy-4-(3-(4-(cyclopropanecarbonyl)octahydropyrrolo[3,2-b]pyrrole-1-carbonyl 4-fluorobenzyl)phthalazin-1(2H)-one

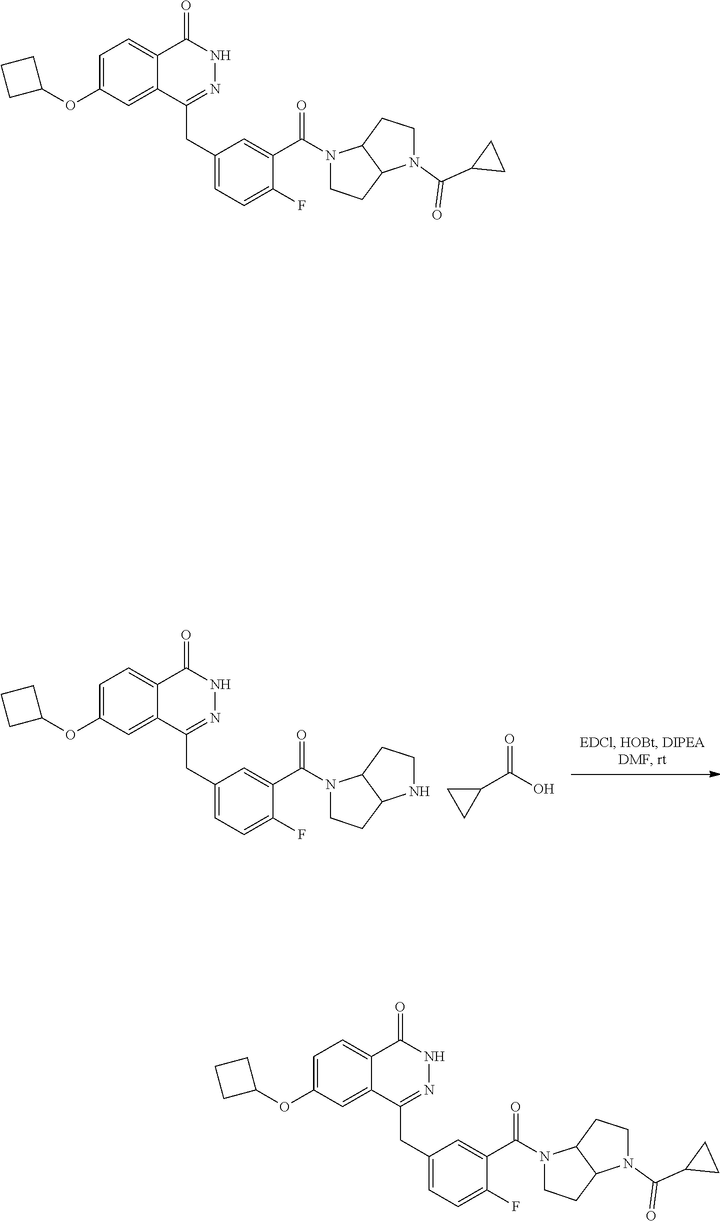

### Step 1

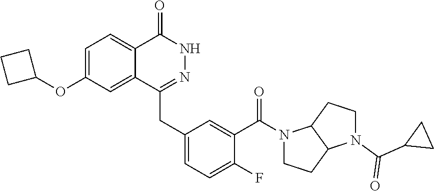 [SMILES: O=C(c1cc(Cc2n[nH]c(=O)c3ccc(OC4CCC4)cc23)ccc1F)N1CCC2C1CCN2C(=O)C1CC1]

### 6-cyclobutoxy-4-(3-(4-(cyclopropanecarbonyl)octahydropyrrolo[3,2-b]pyrrole-1-carbonyl)-4-fluorobenzyl)phthalazin-1(2H)-one

To a solution of 6-cyclobutoxy-4-(4-fluoro-3-(octahydropyrrolo[3,2-b]pyrrole-1-carbonyl)benzyl)phthalazin-1(2H)-one (150 mg, 0.30 mmol) and cyclopropanecarboxylic acid (26 mg, 0.30 mmol) in DMF (5 mL) was added EDCI (86 mg, 0.45 mmol). HOBt (61 mg, 0.45 mmol) and DIPEA (155 mg, 1.2 mmol). The mixture was stirred at rt for 16 hours. The reaction was purified by prep-HPLC to give 6-cyclobutoxy-4-(3-(4-cyclopropanecarbonyl)octahydropyrrolo[3,2-b]pyrrole-1-carbonyl)-4-fluorobenzyl)phthalazin-1(2H)-one (56 mg 38% yield) as a white solid. LCMS ESI m/z 531.2 [M+H]⁺. ¹H NMR (400 MHz, DMSO-d₆) δ 12.46 (s, 1H), 8.15 (m, 1H), 7.57-7.17 (m, 4H), 7.11 (m, 1H), 4.91-4.66 (m, 2H), 4.50 (m, 1H), 4.30 (s, 2H), 3.90 (m, 1H), 3.48 (m, 1H), 3.26-3.02 (m, 2H), 2.37 (t, J=13.3 Hz, 2H), 2.26-2.07 (m, 2H), 2.00 (d, J=5.5 Hz, 3H), 1.92-1.56 (m, 4H), 0.87-0.65 (m, 4H).

### Synthesis of Example 110: 6-Cyclobutoxy-4-(3-(3-((1,5-dimethyl-1H-imidazol-2-yl)(methyl)amino)azetidine-1-carbonyl)-4-fluorobenzyl)phthalazin-1(2H)-one

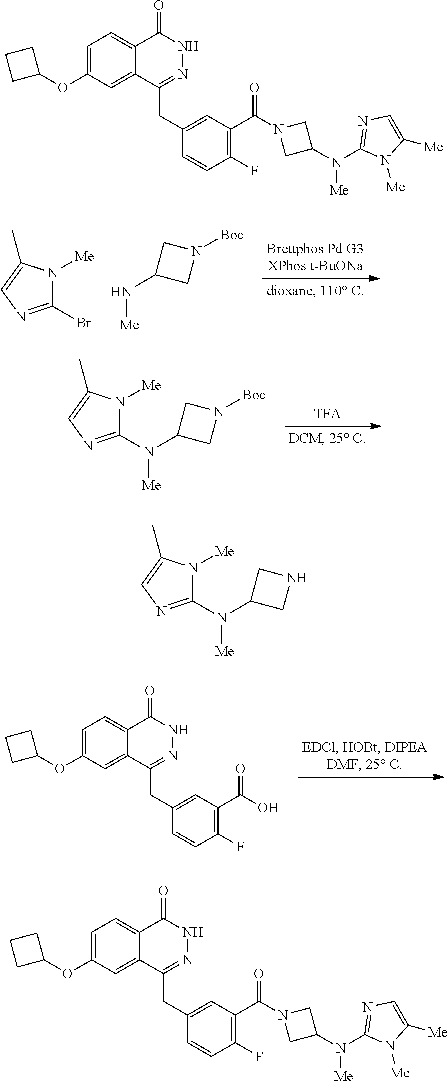

### Step 1

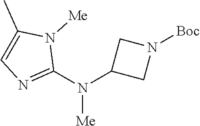 [SMILES: Cc1cnc(N(C)C2CN(C(=O)OC(C)(C)C)C2)n1C]

### tert-Butyl 3-((1,5-dimethyl-1H-imidazol-2-yl)(methyl)amino)azetidine-1-carboxylate

A mixture of 2-bromo-1,5-dimethyl-1H-imidazole (180 mg, 1.03 mmol), tert-butyl 3-(methylamino)azetidine-1-carboxylate (2.87 g, 15.43 mmol), brettphos Pd G3 (93 mg, 0.10 mmol), Xphos (196 mg, 0.41 mmol) and t-BuONa (197 mg, 2.06 mmol) in dioxane (3 mL) was was stirred at 110° C. under Ar (g) for 16 hours. The mixture was cooled to rt, diluted with H₂O (10 mL) and extracted with EtOAc (20 mL×3). The combined organic layer was washed with brine, dried over anhydrous Na₂SO₄, filtered and concentrated in vacuo. The residue was purified by silica gel chromatography (EtOAc/pet ether=30 to 50%) to give tert-butyl 3-((1,5-dimethyl-1H-imidazol-2-yl)(methyl)amino)azetidine-1-carboxylate as a white solid (80 mg, 27% yield). LCMS ESI m/z: 281 [M+H]⁺.

### Step 2

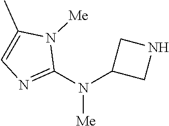

### N-(Azetidin-3-yl)-N-1,5-trimethyl-1H-imidazol-2-amine

To a solution of tert-butyl 3-((1,5-dimethyl-1H-imidazol-2-yl)(methyl)amino)azetidine-1-carboxylate (80 mg, 0.28 mmol) in DCM (1 mL) was added TFA (472 mg, 0.414 mmol). The mixture was stirred at rt for 1 hour. Solvent was removed in vacuo to afford N-azetidin-3-yl)-N-1,5-trimethyl-1H-imidazol-2-amine as a white solid (72 mg, 96% yield). LCMS ESI m/z: 181 [M+H]⁺. This was used without further purification.

### Step 3

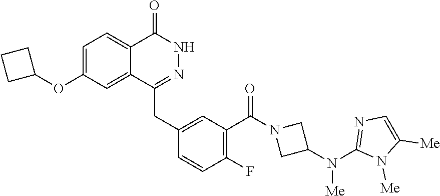 [SMILES: Cc1cnc(N(C)C2CN(C(=O)c3cc(Cc4n[nH]c(=O)c5ccc(OC6CCC6)cc45)ccc3F)C2)n1C]

### 6-Cyclobutoxy-4-(3-(3-((1,5-dimethyl-1H-imidazol-2-yl)(methyl)amino)azetidine-1-carbonyl)-4-fluorobenzyl)phthalazin-1(2H)-one

To a solution of N-(azetidin-3-yl)-N-1,5-trimethyl-1H-imidazol-2-amine (47 mg, 0.26 mmol) and 54(7-cyclobutoxy-4-oxo-3,4-dihydrophthalazin-1-yl)methyl)-2-fluorobenzoic acid (80 mg, 0.22 mmol) in DMF (2 mL) was added EDCI (63 mg, 0.33 mmol). HOBt (44 mg, 0.33 mmol) and DIPEA (84 mg, 0.65 mmol). The reaction mixture was stirred at rt for 1 hour. The reaction was purified by prep-HPLC to give 6-cyclobutoxy-4-(3-(3-((1,5-dimethyl-1H-imidazol-2-yl)(methyl)amino)azetidine-1-carbonyl)-4-fluorobenzyl) phthalazin-1(2H)-one (31 mg, 26% yield) as a white solid. LCMS ESI m/z: 531 [M+H]⁺. ¹H-NMR (400 MHz, DMSO-D6) δ 12.46 (s, 1H), 8.15 (d, J=8.8 Hz, 1H), 7.48-7.38 (m, 2H), 7.30 (dd, J=8.8, 2.3 Hz, 1H), 7.25-7.16 (m, 1H), 7.07 (d, J=2.3 Hz, 1H), 6.33 (s, 1H), 4.90-4.77 (m, 1H), 4.37-4.24 (m, 2H), 4.17-3.95 (m, 3H), 3.82-3.63 (m, 2H), 3.32 (s, 3H), 2.52 (s, 3H), 2.42-2.30 (m, 2H), 2.08 (s, 3H), 2.05-1.90 (m, 2H), 1.85-1.55 (m, 2H).

### Synthesis of Example 111: 6-(4-(5-((7-Cyclobutoxy-4-oxo-3,4-dihydrophthalazin-1-yl)methyl)-2-fluorobenzoyl)hexahydropyrrolo[3,2-b]pyrrol-1(2H)-yl)nicotinonitrile

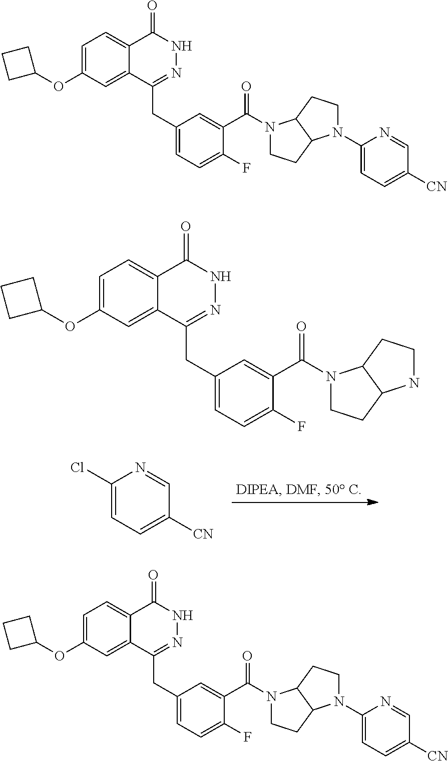 [SMILES: N#Cc1ccc(N2CCC3C2CCN3C(=O)c2cc(Cc3n[nH]c(=O)c4ccc(OC5CCC5)cc34)ccc2F)nc1 ; O=C(c1cc(Cc2n[nH]c(=O)c3ccc(OC4CCC4)cc23)ccc1F)N1CCC2NCCC21 ; N#Cc1ccc(Cl)nc1 ; N#Cc1ccc(N2CCC3C2CCN3C(=O)c2cc(Cc3n[nH]c(=O)c4ccc(OC5CCC5)cc34)ccc2F)nc1]

### Step 1

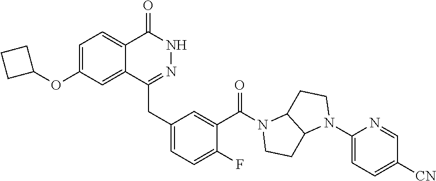 [SMILES: N#Cc1ccc(N2CCC3C2CCN3C(=O)c2cc(Cc3n[nH]c(=O)c4ccc(OC5CCC5)cc34)ccc2F)nc1]

### 6-(4-(5-((7-Cyclobutoxy-4-oxo-3,4-dihydrophthalazin-1-yl)methyl)-2-fluorobenzoyl)hexahydropyrrolo[3,2-b]pyrrol-1(2H)-yl)nicotinonitrile

A mixture of 6-cyclobutoxy-4-(4-fluoro-3-(octahydropyrrolo[3,2-b]pyrrole-1-carbonyl)benzyl)phthalazin-1(2H)-one (150 mg, 0.30 mmol), 6-chloronicotinonitrile (42 mg, 0.30 mmol) and DIPEA (155 mg, 1.20 mmol) in DMF (2 mL) was stirred at 50° C. for 1 hour. The mixture was cooled to rt, diluted with H₂O (10 mL) and extracted with EtOAc (20 mL×3). The combined organic layer was washed with brine, dried over anhydrous Na₂SO₄, filtered and concentrated in vacuo. The residue was purified by prep-HPLC to give 6-(4-(5-((7-cyclobutoxy-4-oxo-3,4-dihydrophthalazin-1-yl)methyl)-2-fluorobenzoyl)hexahydropyrrolo[3,2-b]pyrrol-1(2H)-yl)nicotinonitrile (32 mg, 18% yield) as a white solid. LCMS ESI m/z: 565 [M+H]⁺. ¹H-NMR (400 MHz, DMSO-d₆) δ 12.46 (s, 1H), 8.52 (d, J=2.0 Hz, 1H), 8.15 (d, J=3.9 Hz, 1H), 7.87 (dd, J=9.0, 2.3 Hz, 1H), 7.54-7.34 (m, 2H), 7.29 (dd, J=8.8, 2.3 Hz, 1H), 7.22 (t, J=9.1 Hz, 1H), 7.11 (d, J=2.3 Hz, 1H), 6.69 (d, J=9.0 Hz, 1H), 4.90-4.55 (m, 3H), 4.29 (s, 2H), 3.93-3.76 (m, 1H), 3.45-3.37 (m, 1H), 3.27-3.18 (m, 2H), 2.44-2.31 (m, 2H), 2.31-2.11 (m, 2H), 2.10-1.86 (m, 4H), 1.82-1.70 (m, 1H), 1.69-1.55 (m, 1H).

### Synthesis of Example 112: 6-Cyclobutoxy-4-(3-(3-(cyclopropanecarbonyl)-3,8-diazabicyclo[3,2,1]octane-8-carbonyl)-4-fluorobenzyl)phthalazin-1(2H)-one

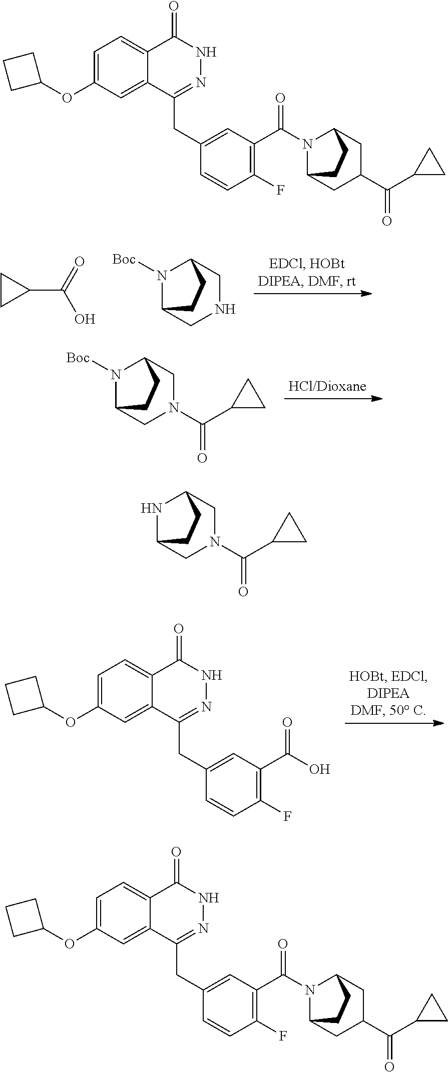

### Step 1

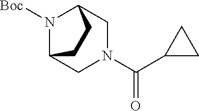

### tert-Butyl 3-(cyclopropanecarbonyl)-3,8-diazabicyclo[3.2.1]octane-8-carboxylate

A mixture of tert-butyl 3,8-diazabicyclo[3.2.1]octane-carboxylate (110 mg, 0.52 mmol) and cyclopropanecarboxylic acid (54 mg, 0.62 mmol) in DMF (2 m L) was added EDCI (149 mg, 0.78 mmol). HOBT (105 mg, 0.78 mmol) and DIPEA (201 mg, 1.55 mmol). The reaction mixture was stirred at rt for 16 hours, then quenched with water (10 mL) and extracted with DCM (50 mL). The organic phase was dried over Na₂SO₄, filtered and concentrated under reduced pressure. The residue was purified by silica gel chromatography (PE/EA=2/1) to give 6-(4-(3-formylbenzoyl)piperazin-1-yl)nicotinonitrile (141 mg, 0.45 mmol, 97% yield) as a colorless oil. LCMS ESI m/z: 281.0 [M+H]⁺.

### Step 2

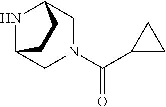

### (3,8-Diazabicyclo[3.2.1]octan-3-yl)(cyclopropyl)methanone

To a solution of tert-butyl 3-(cyclopropanecarbonyl)-3,8-diazabicyclo[3.2.1]octane-8-carboxylate (141 mg, 0.50 mmol) in dioxane (5 mL) was added HCl/dioxane (5 mL) and stirred at 25° C. for 1 hour. The mixture was concentrated in vacuo to afford (3,8-diazabicyclo[3.2.1]octan-3-yl)(cyclopropyl)methanone (90 mg, 0.42 mmol, 83% yield) as a white solid. This was used without further purification. LCMS ESI m/z: 181.0 [M+H]⁺.

### Step 3

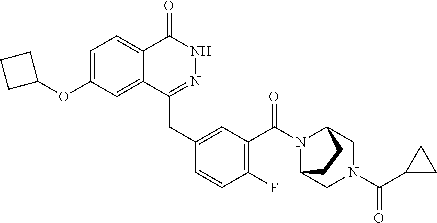

### 6-Cyclobutoxy-4-(3-(3-(cyclopropanecarbonyl)-3,8-diazabicyclo[3.2.1]octane-8-carbonyl)-4-fluorobenzyl)phthalazin-1(2H)-one

To a solution of (3,8-diazabicyclo[3.2.1]octan-3-yl)(cyclopropyl)methanone (50 mg, 0.28 mmol) and 5-((7-cyclobutoxy-4-oxo-3,4-dihydrophthalazin-1-yl)methyl)-2-fluorobenzoic acid (102 mg, 0.28 mmol) in DMF (2 mL) was added EDCI (80 mg, 0.42 mmol), HOBt (56 mg, 0.42 mmol) and DIPEA (108 mg, 0.83 mmol). The reaction mixture was stirred at rt for 16 hours. The mixture was poured into water (10 mL) and extracted with ethyl acetate (20 mL×3). The combined organic layer was washed with brine, dried over Na₂SO₄ and concentrated in vacuo. The residue was purified by prep-HPLC to give 6-cyclobutoxy-4-(3-(3-(cyclopropanecarbonyl)-3,8-diazabicyclo[3.2.1]octane-8-carbonyl)-4-fluorobenzyl)phthalazin-1(2H)-one (70 mg, 0.13 mmol, 48% yield) as a white solid. LCMS ESI m/z: 531.2 [M+H]⁺. ¹H NMR (400 MHz, DMSO-D6) δ 12.45 (s, 1H), 8.14 (d, J=5.8 Hz, 1H), 7.43 (s, 2H), 7.28 (dd, J=16.1, 8.6 Hz, 2H), 7.10 (s, 1H), 4.89-4.79 (m, 1H), 4.69 (s, 1H), 4.31 (s, 2H), 4.25-3.90 (m, 2H), 3.72 (s, 1H), 3.15-2.75 (m, 2H), 2.50 (dt, J=3.5, 1.7 Hz, 4H), 2.39 (d, J=6.5 Hz, 2H), 2.05-1.90 (m, 2H), 1.87-1.71 (m, 2H), 1.65 (dd, J=18.3, 8.9 Hz, 1H), 1.56-1.42 (m, 1H), 0.74 (s, 4H).

### Synthesis of Example 113: N-(1-(5-((7-Cyclobutoxy-4-oxo-3,4-dihydrophthalazin-1-yl)methyl)-2-fluorobenzoyl)azetidin-3-yl)

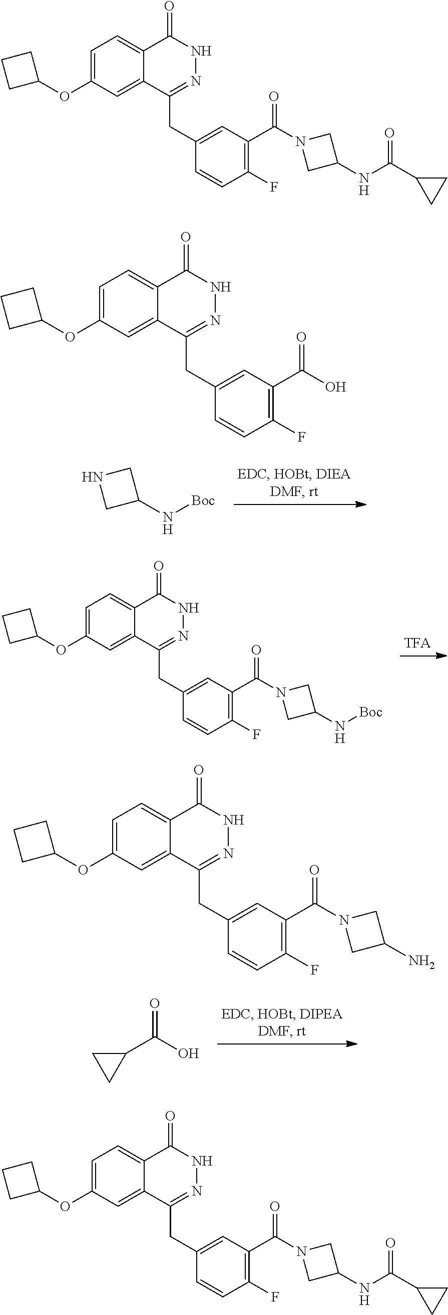

### Step 1

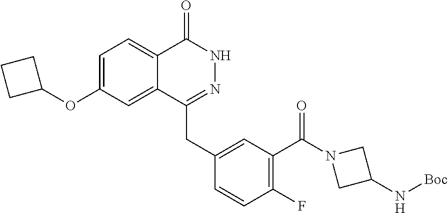 [SMILES: CC(C)(C)OC(=O)NC1CN(C(=O)c2cc(Cc3n[nH]c(=O)c4ccc(OC5CCC5)cc34)ccc2F)C1]

### tert-butyl (1-(5-((7-cyclobutoxy-4-oxo-3,4-dihydrophthalazin-1-yl)methyl)-2-fluoro benzoyl)azetidin-3-yl)carbamate

To a solution of 5-((7-cyclobutoxy-4-oxo-3,4-dihydrophthalazin-1-yl)methyl)-2-fluorobenzoic acid (2(0) mg, 0.54 mmol) and tert-butyl azetidin-3-ylcarbamate (94 mg, 0.54 mmol) in DMF (2 mL) was added EDCI (109 mg, 0.54 mmol). HOBt (110 mg, 0.81 mmol) and DIPEA (351 mg, 2.70 mmol). The reaction mixture was stirred at rt for 16 hours. The mixture was diluted with H₂O (50 mL) and extracted with EtOAc (50 mL×3). The combined organic layer was washed with brine, dried over anhydrous Na₂SO₄, filtered and concentrated in vacuo. The residue was purified by silica gel chromatography (EtOAc/petroleum ether=40% to 45%) to give tert-butyl (0-(5-((7-cyclobutoxy-4-oxo-3,4-dihydrophthalazin-1-yl)methyl)-2-fluorobenzoyl)azetidin-3-yl)carbamate (263 mg 93% yield) as a white solid. LCMS ESI 491.2 [M+H]⁺.

### Step 2

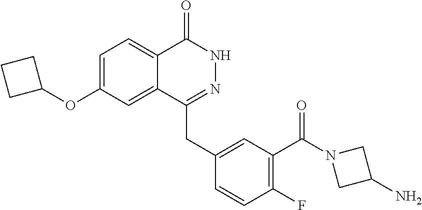 [SMILES: NC1CN(C(=O)c2cc(Cc3n[nH]c(=O)c4ccc(OC5CCC5)cc34)ccc2F)C1]

### 4-(3-(3-aminoazetidine-1-carbonyl)-4-fluorobenzyl)-6-cyclobutoxyphthalazin-1(2H)-one

To a solution of tert-butyl (1-(5-((7-cyclobutoxy-4-oxo-3,4-dihydrophthalazin-1-yl)methyl)-2-fluorobenzoyl)azetidin-3-yl)carbamate (260 mg, 0.50 mmol) in DCM (5 mL) was added TFA (0.5 mL). The mixture was stirred at rt for 1 hour. The solution was concentrated in vacuo to give 4-(3-(3-aminoazetidine-1-carbonyl)-4-fluorobenzyl)-4-cyclobutoxyphthalazin-1(2H)-one (200 mg 95% yield) as a colorless oil. This was used without further purification. LCMS ESI 423 [M+H]⁺.

### Step 3

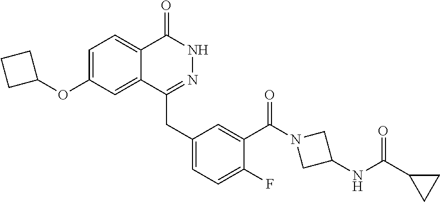 [SMILES: O=C(NC1CN(C(=O)c2cc(Cc3n[nH]c(=O)c4ccc(OC5CCC5)cc34)ccc2F)C1)C1CC1]

### N-(1-(5-((7-Cyclobutoxy-4-oxo-3,4-dihydrophthalazin-1-yl)methyl)-2-fluorobenzoyl)azetidin-3-yl)cyclopropanecarboxamide

To a solution of 4-(3-(3-aminoazetidine-1-carbonyl)-4-fluorobenzyl)-6-cyclobutoxyphthalazin-1(2H)-one (100 mg, 0.19 mmol) and cyclopropanecarboxylic acid (16 mg, 0.19 mmol) in DMF (5 mL) was added EDCI (54 mg, 0.28 mmol), HOBt (38 mg, 0.28 mmol) and DIPEA (96 mg, 0.75 mmol). The mixture was stirred at rt for 16 hours. The reaction was purified by prep-HPLC to give N-(1-(5-((7-cyclobutoxy-4-oxo-3,4-dihydrophthalazin-1-yl)methyl)-2-fluorobenzoyl)azetidin-3-yl)cyclopropanecarboxamide (64 mg 95% yield) as a white solid. LCMS ESI 491.2 [M+H]⁺. ¹H NMR (400 MHz, DMSO-D6) δ 12.47 (s, 1H), 8.75 (m, 1H), 8.15 (m, 1H), 7.52-7.37 (m, 2H), 7.33-7.17 (m, 2H), 7.09 (m, 1H), 4.84 (m, 1H), 4.51 (m 1H), 4.34-4.10 (m, 4H), 3.89-3.73 (m, 2H), 2.37 (d, J=3.4 Hz, 2H), 2.00 (m, 2H), 1.82-1.59 (m, 2H), 1.49 (m 1H), 0.73-0.63 (m, 4H).

### Synthesis of Example 114: N-(1-(5-((7-cyclobutoxy-4-oxo-3,4-dihydrophthalazin-1-yl)methyl)-2-fluorobenzoyl)azetidin-3-yl)-4,4,4-trifluoro-N-methylbutanamide

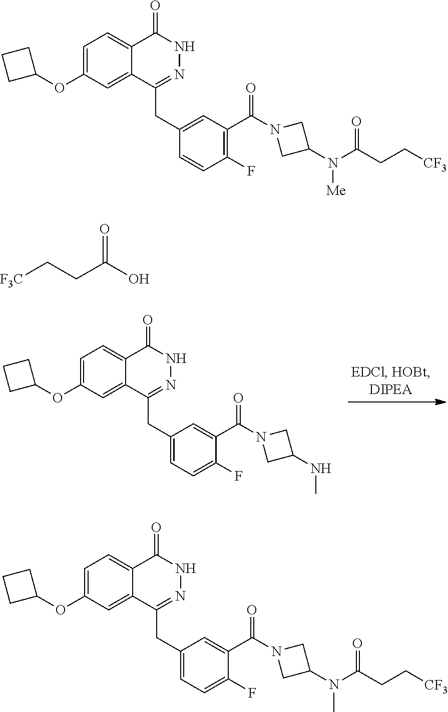 [SMILES: CN(C(=O)CCC(F)(F)F)C1CN(C(=O)c2cc(Cc3n[nH]c(=O)c4ccc(OC5CCC5)cc34)ccc2F)C1 ; O=C(O)CCC(F)(F)F ; CNC1CN(C(=O)c2cc(Cc3n[nH]c(=O)c4ccc(OC5CCC5)cc34)ccc2F)C1 ; CN(C(=O)CCC(F)(F)F)C1CN(C(=O)c2cc(Cc3n[nH]c(=O)c4ccc(OC5CCC5)cc34)ccc2F)C1]

### Step 1

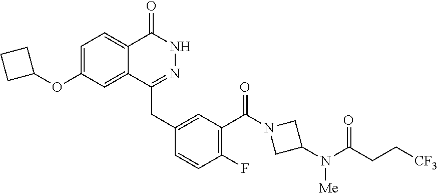 [SMILES: CN(C(=O)CCC(F)(F)F)C1CN(C(=O)c2cc(Cc3n[nH]c(=O)c4ccc(OC5CCC5)cc34)ccc2F)C1]

### N-(1-(5-((7-Cyclobutoxy-4-oxo-3,4-dihydrophthalazin-1-yl)methyl)-2-fluorobenzoyl)azetidin-3-yl)-4,4,4-trifluoro-N-methylbutanamide

To a solution of 6-cyclobutoxy-4-(4-fluoro-3-(3-(methylamino)azetidine-1-carbonyl)benzyl)phthalazin-1(2H)-one (75 mg, 0.17 mmol) and 4,4,4-trifluorobutanoic acid (49 mg, 0.34 mmol) in DMF (2 mL) was added EDCI (49 mg, 0.26 mmol), HOBt (35 mg, 0.26 mmol) and DIPEA (111 mg, 0.86 mmol). The reaction mixture was stirred at 45° C. for 2 hours. The mixture was concentrated in vacuo and the residue was purified by prep-HPLC to afford N-(1-(5-((7-cyclobutoxy-4-oxo-3,4-dihydrophthalazin-1-yl)methyl)-2-fluorobenzoyl)azetidin-3-yl)-4,4,4-trifluoro-N-methylbutanamide as a white solid (50 mg, 52% yield). LCMS ESI m/z 561 (M+H)⁺. ¹H-NMR (400 MHz, DMSO-d₆) δ 12.46 (s, 1H), 8.15 (d, J=8.8 Hz, 1H), 7.51-7.47 (m, 1H), 7.47-7.42 (m, 1H), 7.32-7.28 (m, 1H), 7.27-7.20 (m, 1H), 7.12-7.09 (m, 1H), 5.17-4.94 (m, 1H), 4.88-4.80 (m, 1H), 4.31 (s, 2H), 4.21-3.99 (m, 4H), 3.01 (s, 2H), 2.91 (s, 1H), 2.63-2.56 (m, 2H), 2.47-2.32 (m, 4H), 2.03-1.94 (m, 2H), 1.83-1.74 (m, 1H), 1.70-1.61 (m, 1H).

### Synthesis of Example 115: 6-(5-(((7-Cyclobutoxy-4-oxo-3,4-dihydrophthalazin-1-yl)methyl)-2-fluorobenzoyl) hexahydropyrrolo[3,4-c]pyrrol-2(1H)-yl)nicotinonitrile

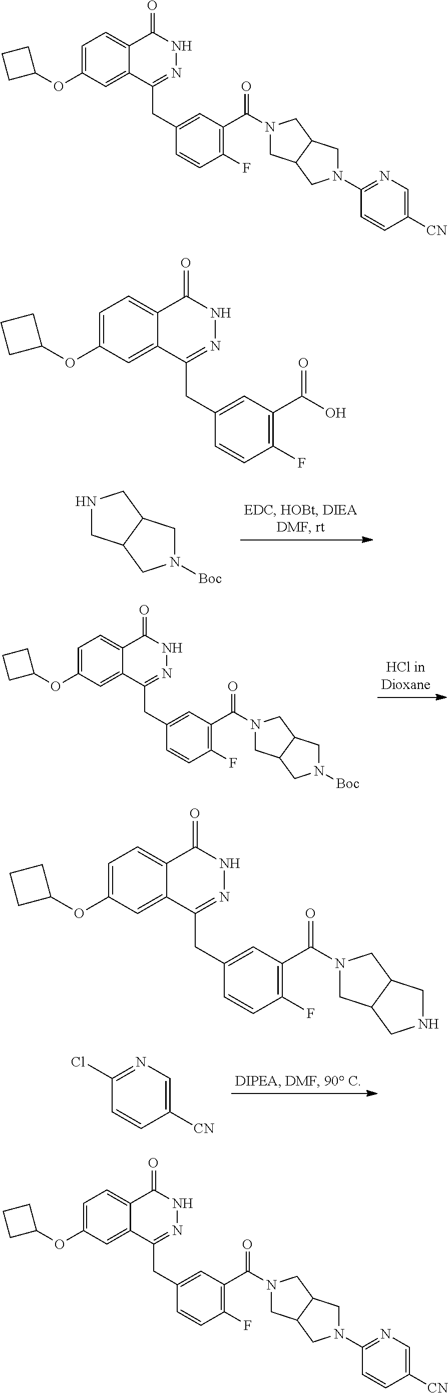

### Step 1

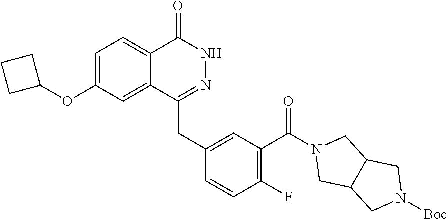 [SMILES: CC(C)(C)OC(=O)N1CC2CN(C(=O)c3cc(Cc4n[nH]c(=O)c5ccc(OC6CCC6)cc45)ccc3F)CC2C1]

### tert-Butyl 5-(5-((7-cyclobutoxy-4-oxo-3,4-dihydrophthalazin-1-yl)methyl)-2-fluorobenzoyl)hexahydropyrrolo[3,4-c]pyrrole-2(1H)-carboxylate

To a solution of 5-((7-cyclobutoxy-4-oxo-3,4-dihydrophthalazin-1-yl)methyl)-2-fluorobenzoic acid (300 mg, 0.81 mmol) and tert-butyl hexahydropyrrolo[3,4-c]pyrrole-2(1H)-carboxylate (207 mg, 0.98 mmol) in DMF (2 mL) was added EDCI (234 mg, 1.22 mmol), HOBt (165 mg, 1.22 mmol) and DIPEA (526 mg, 4.07 mmol). The reaction mixture was stirred at rt for 16 hours. The mixture was diluted with H₂O (50 mL) and extracted with EtOAc (50 mL×3). The combined organic layer was washed with brine, dried over anhydrous Na₂SO₄, filtered and concentrated in vacuo. The residue was purified by silica gel chromatography (EtOAc/petroleum ether=40% to 50%) to give tert-butyl 5-(5-((7-cyclobutoxy-4-oxo-3,4-dihydrophthalazin-1-yl)methyl)-2-fluorobenzoyl)hexa hydropyrrolo[3,4-c]pyrrole-2(1H)-carboxylate (436 mg 95% yield) as a white solid. LCMS ESI 563 [M+H]⁺.

### Step 2

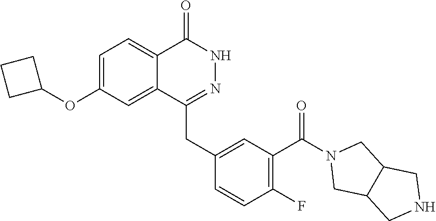 [SMILES: O=C(c1cc(Cc2n[nH]c(=O)c3ccc(OC4CCC4)cc23)ccc1F)N1CC2CNCC2C1]

### 6-Cyclobutoxy-4-(4-fluoro-3-(octahydropyrrolo[3,4-c]pyrrole-2-carbonyl)benzyl)phthalazin-1(2H)-one

To a solution of tert-butyl 5-(5-((7-cyclobutoxy-4-oxo-3,4-dihydrophthalazin-1-yl)methyl)-2-fluorobenzoyl)hexahydropyrrolo[3,4-c]pyrrole-2(1H)carboxylate (436 mg, 0.75 mmol)) in dioxane (2 mL) was added HCl/dioxane (4.0M, 4 mL, 16.00 mmol). The mixture was stirred at rt for 1 hour. The solvent was removed in vacuo to give 6-cyclobutoxy-4-(4-fluoro-3-(octahydropyrrolo[3,4-c]pyrrole-2-carbonyl)benzyl)phthalazin-1(2H)-one (322 mg 90% yield) as a white solid. This was used without further purification. LCMS ESI m/z 463 [M+H]⁺.

### Step 3

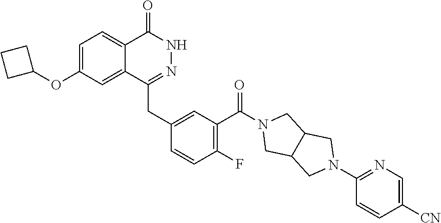 [SMILES: N#Cc1ccc(N2CC3CN(C(=O)c4cc(Cc5n[nH]c(=O)c6ccc(OC7CCC7)cc56)ccc4F)CC3C2)nc1]

### 6-(5-(5-((7-Cyclobutoxy-4-oxo-3,4-dihydrophthalazin-1-yl)methyl)-2-fluorobenzoyl) hexahydropyrrolo[3,4-c]pyrrol-2(1H)-yl)nicotinonitrile

To a solution of 6-cyclobutoxy-4-(4-fluoro-3-(octahydropyrrolo[3,4-c]pyrrole-2-carbonyl)benzyl)phthalazin-1(2H)-one (60 mg, 0.13 mmol) and 6-chloronicotinonitrile (54 mg, 0.39 mmol) in DMF (1 mL) was added DIPEA (84 mg, 0.65 mmol). The reaction mixture was stirred at 90° C. for 2 hours. The mixture was concentrated in vacuo and the residue was purified by prep-HPLC to give 6-(5-(5-((7-cyclobutoxy-4-oxo-3,4-dihydrophthalazin-1-yl)methyl)-2-fluorobenzoyl)hexahydropyrrolo[3,4-c]pyrrol-2(1H)-yl)nicotinonitrile (35 mg, 47% yield) as a white solid. LCMS ESI m/z: 565 [M+H]⁺. ¹H-NMR (400 MHz, DMSO-d₆) δ 12.45 (s, 1H), 8.49-8.45 (m, 1H), 8.14 (d, J=8.8 Hz, 1H), 7.83 (dd, J=8.9, 2.3 Hz, 1H), 7.41-7.36 (m, 2H), 7.31-7.28 (m, 1H), 7.25-7.19 (m, 1H), 7.08-7.06 (m, 1H), 6.53 (d, J=9.0 Hz, 1H), 4.86-4.78 (m, 1H), 4.29 (s, 2H), 3.81-3.70 (m, 2H), 3.67-3.58 (m, 1H), 3.51-3.40 (m, 3H), 3.30-3.22 (m, 1H), 3.13-3.06 (m, 2H), 3.04-2.97 (m, 1H), 2.41-2.34 (m, 2H), 2.03-1.95 (m, 2H), 1.82-1.73 (m, 1H), 1.69-1.61 (m, 1H).

### Synthesis of Example 116: 6-(3-(5-((7-Cyclobutoxy-4-oxo-3-dihydrophthalazin-1-yl)methyl)-2-fluorobenzoyl)-3,8-diazabicyclo[3.2.1]octan-8-yl)nicotinonitrile

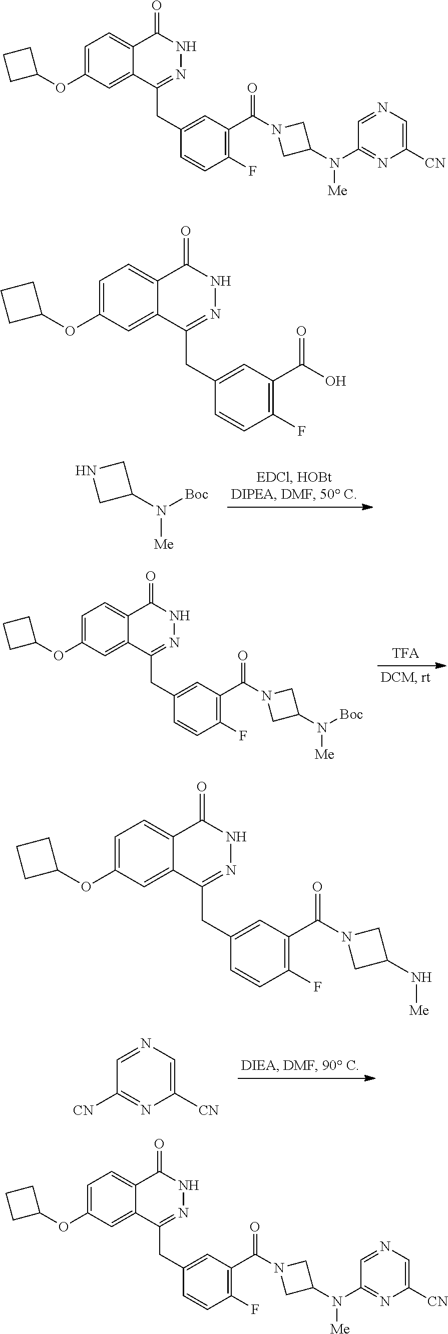

### Step 1

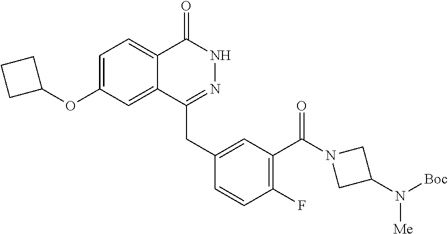 [SMILES: CN(C(=O)OC(C)(C)C)C1CN(C(=O)c2cc(Cc3n[nH]c(=O)c4ccc(OC5CCC5)cc34)ccc2F)C1]

### tert-Butyl (1-(5-((7-cyclobutoxy-4-oxo-3,4-dihydrophthalazin-1-yl)methyl)-2-fluorobenzoyl)azetidin-3-yl)(methyl)carbamate

To a solution of 5-((7-cyclobutoxy-4-oxo-3,4-dihydrophthalazin-1-yl)methyl)-2-fluorobenzoic acid (250 mg, 0.68 mmol) and tert-butyl azetidin-3-yl(methyl)carbamate (139 mg, 0.75 mmol) in DMF (5 mL) was added EDCI (195 mg, 1.02 mmol), HOBt (138 mg, 1.02 mmol) and DIPEA (263 mg, 2.04 mmol). The reaction mixture was stirred at rt for 16 hours, then the reaction was quenched with H₂O (10 mL) and extracted with EtOAc (10 mL×3). The organic phase was washed with sat. NaCl solution, dried over Na₂SO₄, filtered and concentrated under reduced pressure. The crude product was purified by silica gel chromatography (Petroleum ether/EtOAc=2/1) to give 6-(4-(3-formylbenzoyl)piperazin-1-yl)nicotinonitrile (242 mg, 66% yield) as a white solid. LCMS ESI m/z: 537.1 [M+H]⁺.

### Step 2

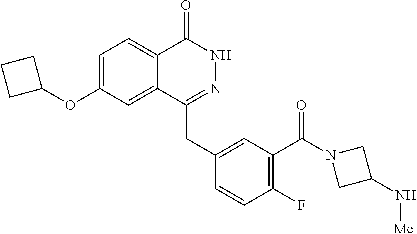 [SMILES: CNC1CN(C(=O)c2cc(Cc3n[nH]c(=O)c4ccc(OC5CCC5)cc34)ccc2F)C1]

### 6-Cyclobutoxy-4-(4-fluoro-3-(3-(methylamino)azetidine-1-carbonyl)benzyl)phthalazin-1(2H)-one

To a solution of tert-butyl (1-(5-((7-cyclobutoxy-4-oxo-3,4-dihydrophthalazin-1-yl)methyl)-2-fluorobenzoyl)azetidin-3-yl)(methyl)carbamate (222 mg, 0.41 mmol) in DCM (10 mL) was added TFA (1 mL) and stirred at 25° C. for 2 hours. The reaction mixture was concentrated in vacuo to afford 6-cyclobutoxy-4-(4-fluoro-3-(3-(methylamino)azetidine-1-carbonyl)benzyl)phthalazin-1(2H)-one (180 mg, 0.41 mmol, 99% yield) as a yellow oil. This was used without further purification. LCMS ESI m/z: 437.1 [M+H]⁺.

### Step 3

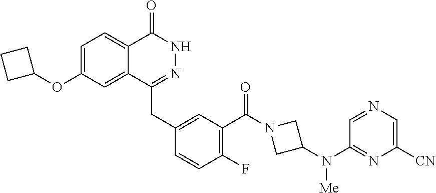 [SMILES: CN(c1cncc(C#N)n1)C1CN(C(=O)c2cc(Cc3n[nH]c(=O)c4ccc(OC5CCC5)cc34)ccc2F)C1]

### 6-((1-(5-((7-Cyclobutoxy-4-oxo-3,4-dihydrophthalazin-1-yl)methyl)-2-fluorobenzoyl) azetidin-3-yl)(methyl)amino)pyrazine-2-carbonitrile

To a solution of 6-cyclobutoxy-4-(4-fluoro-3-(3-(methylamino)azetidine-1-carbonyl)benzyl)phthalazin-1(2H)-one (100 mg, 0.23 mmol) and 6-chloropyrazine-2-carbonitrile (96 mg, 0.69 mmol) in DMF (1 mL) was added DIPEA (81 mg, 0.63 mmol). The reaction mixture was stirred at 90° C. for 2 hours. The reaction was purified by prep-HPLC to give 6-((1-(5-((7-cyclobutoxy-4-oxo-3,4-dihydrophthalazin-1-yl)methyl)-2-fluorobenzoyl)azetidin-3-yl)(methyl)amino)pyrazine-2-carbonitrile (54.5 mg, 54% yield) as a white solid. LCMS ESI m/z: 540.1 [M+H]⁺. ¹H-NMR (40) MHz, DMSO-d6) δ 12.46 (s, 1H), 8.53 (s, 1H), 8.35 (s, 1H), 8.14 (d, J=8.8 Hz, 1H), 7.53 (dd, J=6.4, 2.1 Hz, 1H), 7.47-7.41 (m, 1H), 7.32-7.20 (m, 2H), 7.09 (d, J=2.2 Hz, 1H), 5.33-5.14 (m, 1H), 4.82 (p, J=7.1 Hz, 1H), 4.37-4.27 (m, 3H), 4.24-4.06 (m, 3H), 3.13 (s, 3H), 2.43-2.27 (m, 2H), 2.04-1.90 (m, 2H), 1.74 (dt, J=18.1, 9.1 Hz, 1H), 1.62 (dt, J=18.4, 9.3 Hz, 1H).

### Synthesis of Example 117: N-(1-(5-((7-cyclobutoxy-4-oxo-3,4-dihydrophthalazin-1-yl)methyl)-2-fluorobenzoyl)azetidin-3-yl)-N-methylazetidine-2-carboxamide

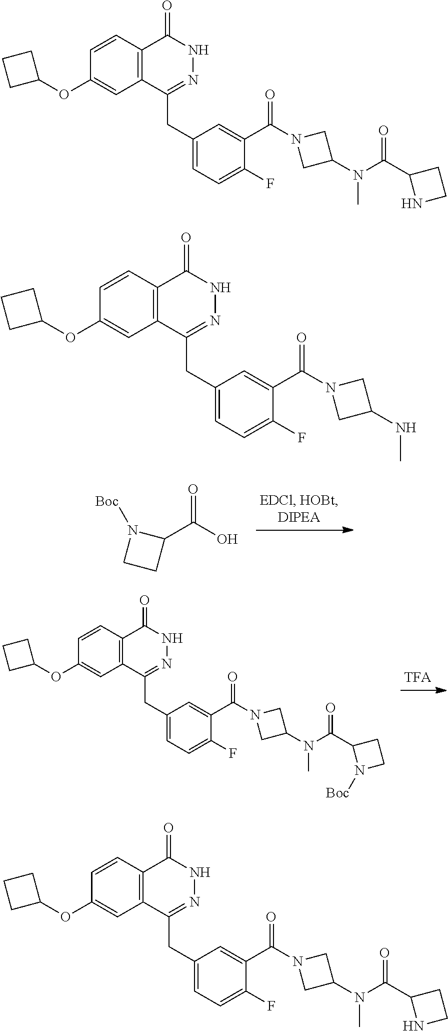

### Step 1

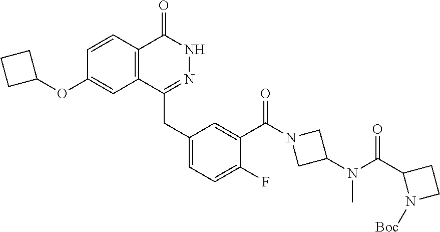 [SMILES: CN(C(=O)C1CCN1C(=O)OC(C)(C)C)C1CN(C(=O)c2cc(Cc3n[nH]c(=O)c4ccc(OC5CCC5)cc34)ccc2F)C1]

### tert-butyl 2-((1-(5-((7-cyclobutoxy-4-oxo-3,4-dihydrophthalazin-1-yl)methyl)-2-fluorobenzoyl)azetidin-3-yl)(methyl)carbamoyl)azetidine-1-carboxylate

To a solution of 6-cyclobutoxy-4-(4-fluoro-3-(3-(methylamino)azetidine-1-carbonyl)benzyl)phthalazin-1(2H)-one (89 mg, 0.20 mmol) and 1-(tert-butoxycarbonyl)azetidine-2-carboxylic acid (50 mg, 0.25 mmol) in DMF (3 mL) was added EDCI (39 mg, 0.20 mmol), HOBt (42 mg, 0.30 mmol) and DIPEA (79 mg, 0.61 mmol). The reaction mixture was stirred at rt for 16 hours. The mixture was diluted with H₂O (10 mL) and extracted with EtOAc (10 mL×3). The combined organic layer was washed with brine, dried over anhydrous Na₂SO₄, filtered and concentrated in vacuo. The residue was purified by silica gel chromatography (EtOAc/pet ether=30 to 50%) to give tert-butyl 2-((1-(5-((7-cyclobutoxy-4-oxo-3,4-dihydrophthalazin-1-yl)methyl)-2-fluorobenzoyl)azetidin-3-yl)(methyl)carbamoyl)azetidine-1-carboxylate as a yellow solid (95 mg, 75% yield). LCMS EST m/z 620 [M+H]⁺.

### Step 2

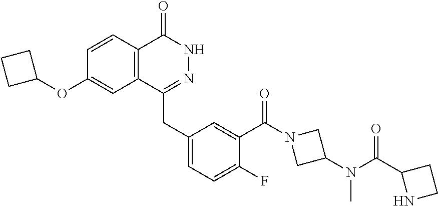 [SMILES: CN(C(=O)C1CCN1)C1CN(C(=O)c2cc(Cc3n[nH]c(=O)c4ccc(OC5CCC5)cc34)ccc2F)C1]

### N-(1-(5-((7-cyclobutoxy-4-oxo-3,4-dihydrophthalazin-1-yl)methyl)-2-fluorobenzoyl)azetidin-3-yl)-N-methylazetidine-2-carboxamide

A solution of ten-butyl 2-((1-(5-((7-cyclobutoxy-4-oxo-3,4-dihydrophthalazin-1-yl)methyl)-2-fluorobenzoyl)azetidin-3-yl)(methyl)carbamoyl)azetidine-1-carboxylate (95 mg, 0.15 mmol) in DCM (5 mL) and TFA (0.5 mL) was stirred at rt for 2 hours. The mixture was concentrated in vacuo and the residue was purified by prep-HPLC to give N-(1-(5-((7-cyclobutoxy-4-oxo-3,4-dihydrophthalazin-1-yl)methyl)-2-fluorobenzoyl)azetidin-3-yl)-N-methylazetidine-2-carboxamide as a white solid (35 mg, 44% yield). LCMS ESI m/z 520 (M+H)⁺. ¹H NMR (400 MHz, DMSO-d₆) δ 12.48 (s, 1H), 8.29 (s, 1H), 8.16 (d, J=8.8 Hz, 1H), 7.55-7.41 (m, 2H), 7.31 (dd, J=8.8, 2.2 Hz, 1H), 7.27-7.19 (m, 1H), 7.10 (t, J=4.1 Hz, 1H), 5.36-5.04 (m, 1H), 4.84 (p, J=7.2 Hz, 1H), 4.72-4.57 (m, 1H), 4.31 (s, 2H), 4.21 (d, J=7.9 Hz, 1H), 4.08 (dd, J=17.3, 10.9 Hz, 4H), 3.55-3.38 (m, 2H), 2.87 (dd, J=36.6, 23.1 Hz, 3H), 2.72-2.57 (m, 1H), 2.38 (d, J=4.3 Hz, 2H), 1.99 (t, J=7.9 Hz, 2H), 1.83-1.60 (m, 2f), 1.23 (s, 1H).

### Synthesis of Example 118: 2-Cyano-N-(1-(5-((7-cyclobutoxy-4-oxo-3,4-dihydrophthalazin-1-yl)methyl)-2-fluorobenzoyl)azetidin-3-yl)-N-methylacetamide

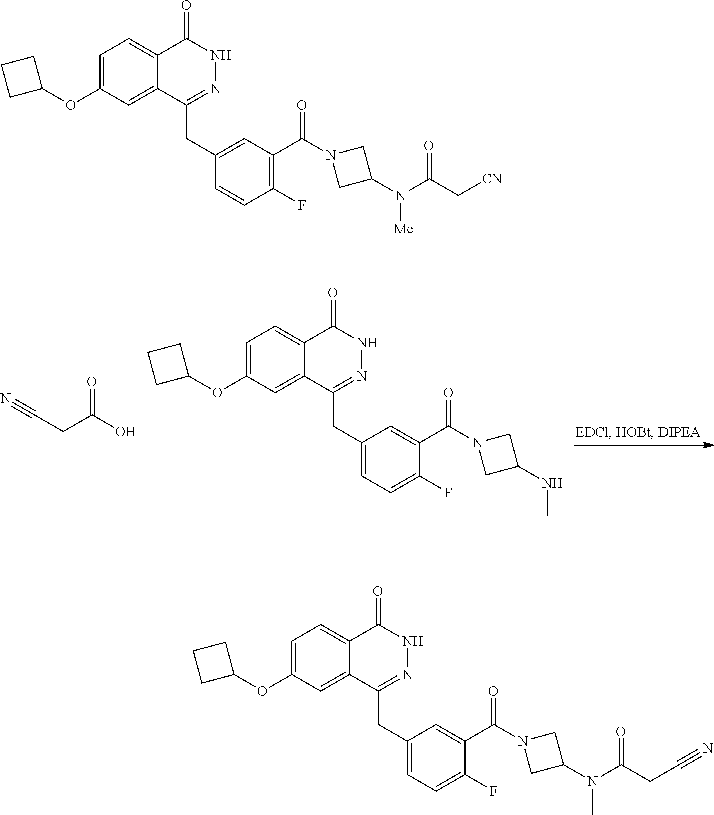 [SMILES: CN(C(=O)CC#N)C1CN(C(=O)c2cc(Cc3n[nH]c(=O)c4ccc(OC5CCC5)cc34)ccc2F)C1 ; N#CCC(=O)O ; CNC1CN(C(=O)c2cc(Cc3n[nH]c(=O)c4ccc(OC5CCC5)cc34)ccc2F)C1 ; CN(C(=O)CC#N)C1CN(C(=O)c2cc(Cc3n[nH]c(=O)c4ccc(OC5CCC5)cc34)ccc2F)C1]

### Step 1

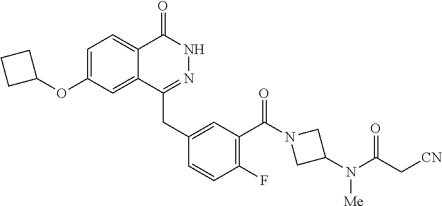 [SMILES: CN(C(=O)CC#N)C1CN(C(=O)c2cc(Cc3n[nH]c(=O)c4ccc(OC5CCC5)cc34)ccc2F)C1]

### 2-Cyano-N-(1-(5-((7-cyclobutoxy-4-oxo-3,4-dihydrophthalazin-1-yl)methyl)-2-fluorobenzoyl)azetidin-3-yl)-N-methylacetamide

To a solution of 2-cyanoacetic acid (75 mg, 0.17 mmol) and 6-cyclobutoxy-4-(4-fluoro-3-(3-(methylamino)azetidine-1-carbonyl)benzyl)phthalazin-1(2H)-one (29 mg, 0.34 mmol) in DMF (1 mL) was added EDCI (49 mg, 0.26 mmol). HOBt (35 mg, 0.26 mmol) and DIPEA (111 mg, 0.86 mmol). The reaction mixture was stirred at rt for 3 hours and purified by prep-HPLC to give 2-cyano-N-(1-(5-((7-cyclobutoxy-4-oxo-3,4-dihydrophthalazin-1-yl)methyl)-2-fluorobenzoyl)azetidin-3-yl)-N-methylacetamide as a white solid (25 mg, 29% yield). LCMS ESI 504 (M+H)⁺. ¹H NMR (400 MHz, CDCl₃) δ 9.90 (s, 1H), 8.35 (d, J=8.8 Hz, 1H), 7.51 (s, 1H), 7.34 (s, 1H), 7.20 (d, J=6.7 Hz, 1H), 7.05 (t, J=9.1 Hz, 1H), 6.90 (s, 1H), 5.25 (s, 1H), 4.79-4.61 (m, 1H), 4.48-4.36 (m, 1H), 4.33-4.08 (m, 5H), 3.52 (s, 2H), 3.14 (d, J=13.4 Hz, 3H), 2.41 (s, 2H), 2.22-2.08 (m, 2H), 1.90 (d, J=10.9 Hz, 1H), 1.83-1.67 (m, 1H).

### Synthesis of Example 119: 6-(4-(5-((7-(cyclopropylmethoxy)-4-oxo-3,4-dihydrophthalazin-1-yl)methyl)-2-fluorobenzoyl)piperazin-1-yl)nicotinonitrile

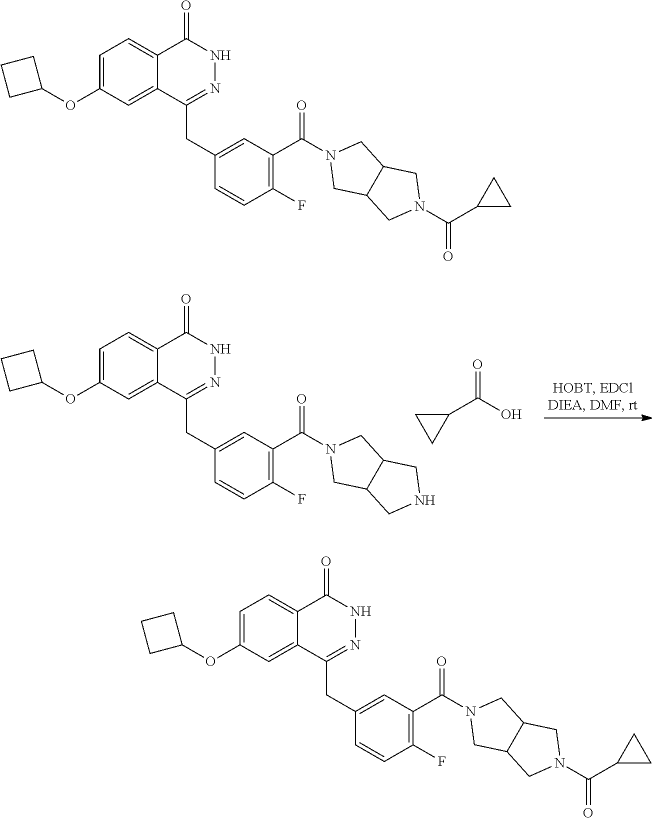

### Step 1

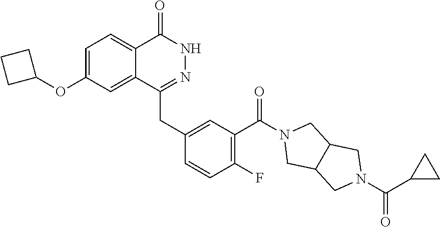 [SMILES: O=C(c1cc(Cc2n[nH]c(=O)c3ccc(OC4CCC4)cc23)ccc1F)N1CC2CN(C(=O)C3CC3)CC2C1]

### 6-Cyclobutoxy-4-(3-(5-(cyclopropanecarbonyl)octahydropyrrolo[3,4-c]pyrrole-2-carbonyl)-4-fluorobenzyl)phthalazin-1(2H)-one

To a solution of cyclopropanecarboxylic acid (17.3 mg, 0.20 mmol) and 6-cyclobutoxy-4-(4-fluoro-3-(octahydropyrrolo[3,4-c]pyrrole-2-carbonyl)benzyl)phthalazin-1(2H)-one (100 mg, 0.20 mmol) in DMF (2 mL) was added EDCI (58 mg, 0.30 mmol). HOBt (41 mg, 0.30 mmol) and DIPEA (104 mg, 0.80 mmol). The reaction mixture was stirred at rt for 16 hours, then purified by prep-HPLC to afford 6-cyclobutoxy-4-(3-(5-(cyclopropanecarbonyl)octahydropyrrolo[3,4-c]pyrrole-2-carbonyl)-4-fluorobenzyl)phthalazin-1(2H)-one (52 mg, 49% yield) as a white solid. LCMS ESI m/z 531.2 [M+H]⁺. ¹H NMR (400 MHz, DMSO-D6) δ 12.45 (s, 1H), 8.15 (m, 1H), 7.40 (m, 2H), 7.30 m, 1H), 7.23 (m, 1H), 7.08 (s, 1H), 4.89-4.78 (m, 1H), 4.29 (s, 2H), 3.90-3.68 (m, 2H), 3.61-3.54 (m, 1H), 3.45 (m, 3H), 3.23-2.81 (m, 4H), 2.39 (s, 2H), 2.00 (d, J=2.6 Hz, 2H), 1.82-1.63 (m, 3H), 0.75-0.67 (m, 4H).

### Synthesis of Example 120: 6-(4-(3-((7-Hydroxy-4-oxo-3,4-dihydrophthalazin-1-yl)methyl)benzoyl)piperazin-1-yl)nicotinonitrile

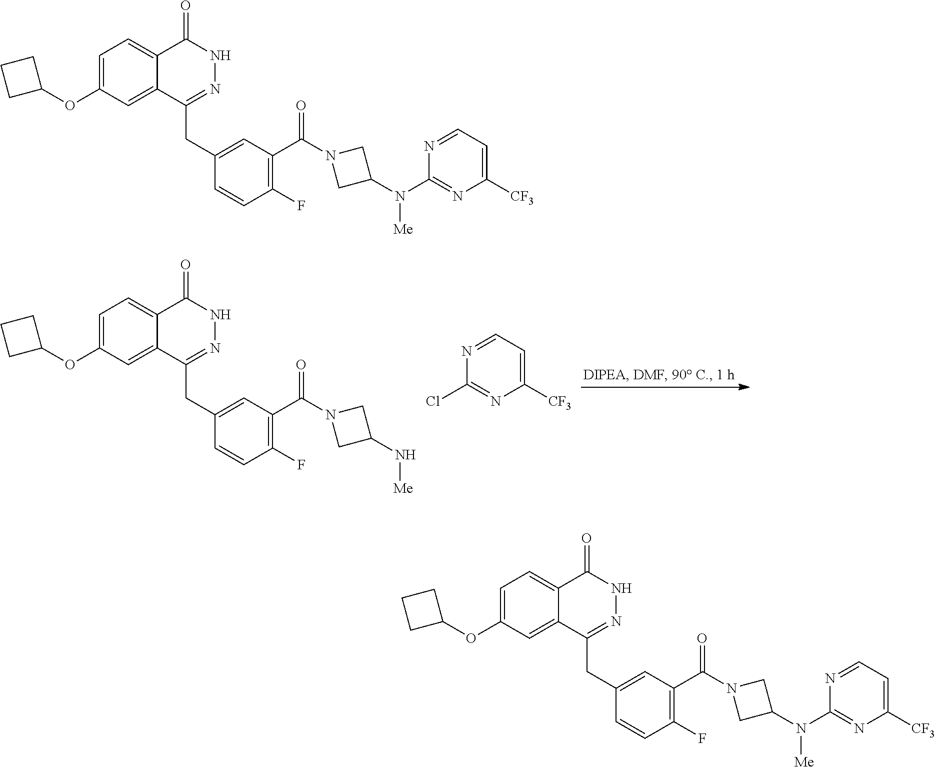 [SMILES: CN(c1nccc(C(F)(F)F)n1)C1CN(C(=O)c2cc(Cc3n[nH]c(=O)c4ccc(OC5CCC5)cc34)ccc2F)C1 ; CNC1CN(C(=O)c2cc(Cc3n[nH]c(=O)c4ccc(OC5CCC5)cc34)ccc2F)C1 ; FC(F)(F)c1ccnc(Cl)n1 ; CN(c1nccc(C(F)(F)F)n1)C1CN(C(=O)c2cc(Cc3n[nH]c(=O)c4ccc(OC5CCC5)cc34)ccc2F)C1]

### Step 1

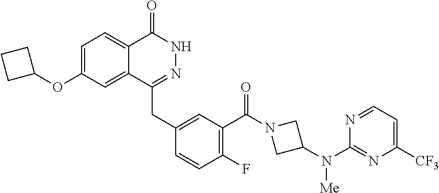 [SMILES: CN(c1nccc(C(F)(F)F)n1)C1CN(C(=O)c2cc(Cc3n[nH]c(=O)c4ccc(OC5CCC5)cc34)ccc2F)C1]

### 6-Cyclobutoxy-4-(fluoro-3-(3-(methyl(4-(trifluoromethyl)pyrimidin-2-yl)amino) azetidine-1-carbonyl)benzyl)phthalazin-1(2H)-one

A mixture of 6-cyclobutoxy-4-(4-fluoro-3-(3-(methylamino)azetidine-1-carbonyl)benzyl) phthalazin-1(2H)-one (55 mg, 0.13 mmol), 2-chloro-4-(trifluoromethyl) pyrimidine (46 mg, 0.26 mmol) in DMF (1 mL) was added DIPEA (49 mg, 0.39 mmol). The reaction was stirred at 90° C. for 1 hour. The reaction was purified by prep-HPLC to give 6-cyclobutoxy-4-(4-fluoro-3-(3-(methyl(4-(trifluoromethyl)pyrimidin-2-yl)amino) azetidine-1-carbonyl)benzyl) phthalazin-1(2H)-one (29 mg, 40% yield) as a white solid. LCMS ESI m/z: 583.0 [M+H]⁺. ¹H NMR (400 MHz, DMSO-D6) δ 12.47 (s, 1H), 8.70 (d, J=4.8 Hz, 1H), 8.14 (d, J=8.8 Hz, 1H), 7.53 (dd, J=6.5, 2.2 Hz, 1H), 7.47-7.41 (m, 1H), 7.31-7.20 (m, 2H), 7.13-7.08 (m, 2H), 5.35 (s, 1H), 4.82 (p, J=7.1 Hz, 1H), 4.41-4.26 (m, 3H), 4.18 (m, 3H), 3.17 (s, 3H), 2.41-2.29 (m, 2H), 2.03-1.89 (m, 2H), 1.82-1.53 (m, 2H).

### Synthesis of Example 121: N-(1-(5-((7-Cyclobutoxy-4-oxo-3,4-dihydrophthalazin-1-yl)methyl)-2-fluorobenzoyl)azetidin-3-yl)-N-methylcyclopropanecarboxamide

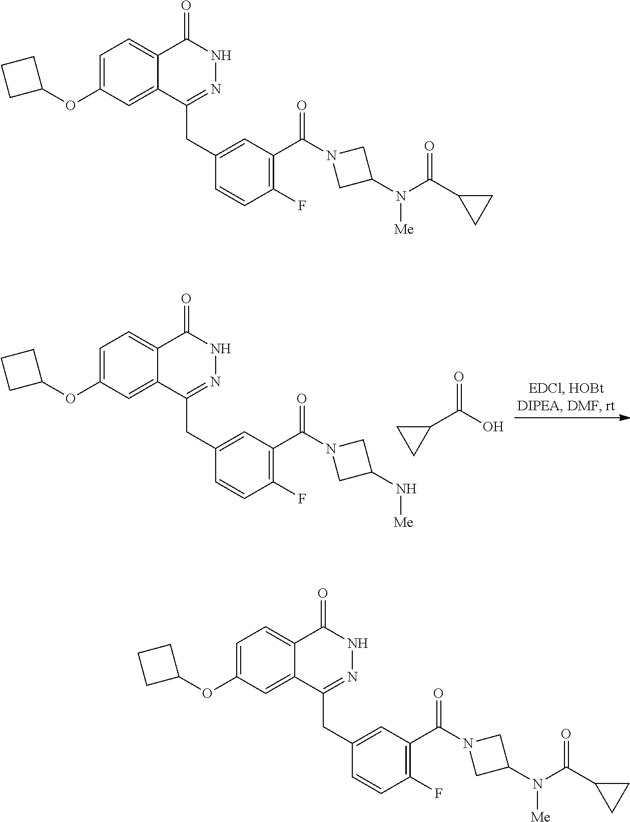

### Step 1

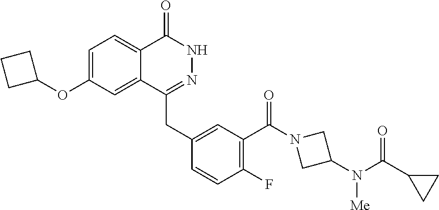 [SMILES: CN(C(=O)C1CC1)C1CN(C(=O)c2cc(Cc3n[nH]c(=O)c4ccc(OC5CCC5)cc34)ccc2F)C1]

### N-(1-(5-((7-cyclobutoxy-4-oxo-3,4-dihydrophthalazin-1-yl)methyl)-2-fluorobenzoyl)azetidin-3-yl)-N-methylcyclopropanecarboxamide

To a solution of cyclopropanecarboxylic acid (40 mg, 0.81 mmol) and 6-cyclobutoxy-4-(4-fluoro-3-(3-(methylamino)azetidine-1-carbonyl)benzyl)phthalazin-1(2H)-one (110 mg, 0.25 mmol) in DMF (2 mL) was added EDCI (62 mg, 0.48 mmol). HOBt (32 mg, 0.24 mmol) and DIPEA (62 mg, 0.48 mmol). The reaction mixture was stirred at rt for 16 hours, then purified by pre-HPLC to afford N-(1-(5-((7-cyclobutoxy-4-oxo-3,4-dihydrophthalazin-1-yl)methyl)-2-fluorobenzoyl)azetidin-3-yl)-N-methylcyclopropane carboxamide (36 mg 28% yield) as a white solid. LCMS EST m/z: 505.1 [M+H]⁺. ¹H NMR (400 MHz, DMSO-D6) δ 12.46 (s, 1H), 8.15 (m, 1H), 7.55-7.48 (m, 1H), 7.43 (s, 1H), 7.30 (m, 1H), 7.25 (d, J=9.0 Hz, 1H), 7.11 (s, 1H), 5.22 (m, 1H), 4.94-4.73 (m, 1H), 4.30 (s, 2H), 4.11 (m, 3H), 3.15 (s, 2H), 2.90 (s, 1H), 2.37 (s, 2H), 2.13-1.17 (m, 6H), 0.72 (s, 4H).

### Synthesis of Example 122: N-(1-(5-((7-cyclobutoxy-4-oxo-3,4-dihydrophthalazin-1-yl)methyl)-2-fluorobenzoyl)azetidin-3-yl)-N-methyloxetane-2-carboxamide

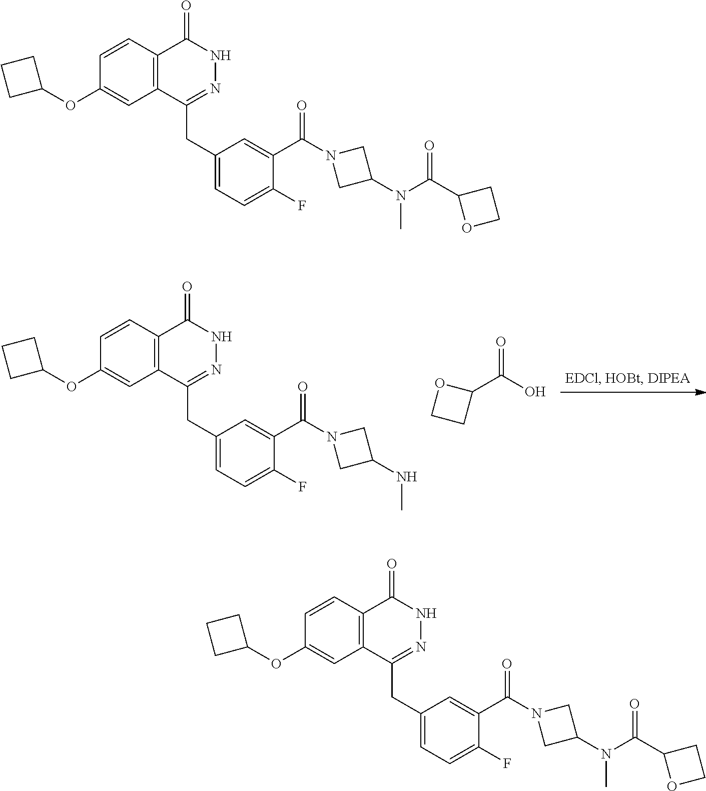 [SMILES: CN(C(=O)C1CCO1)C1CN(C(=O)c2cc(Cc3n[nH]c(=O)c4ccc(OC5CCC5)cc34)ccc2F)C1 ; CNC1CN(C(=O)c2cc(Cc3n[nH]c(=O)c4ccc(OC5CCC5)cc34)ccc2F)C1 ; O=C(O)C1CCO1 ; CN(C(=O)C1CCO1)C1CN(C(=O)c2cc(Cc3n[nH]c(=O)c4ccc(OC5CCC5)cc34)ccc2F)C1]

### Step 1

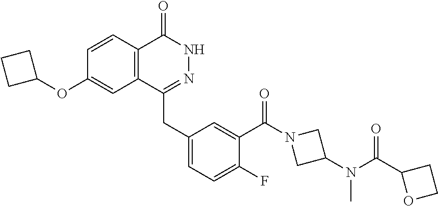 [SMILES: CN(C(=O)C1CCO1)C1CN(C(=O)c2cc(Cc3n[nH]c(=O)c4ccc(OC5CCC5)cc34)ccc2F)C1]

### N-(1-(5-((7-cyclobutoxy-4-oxo-3,4-dihydrophthalazin-1-yl)methyl)-2-fluorobenzoyl)azetidin-3-yl)-N-methyloxetane-2-carboxamide

To a solution of 6-cyclobutoxy-4-(4-fluoro-3-(3-(methylamino)azetidine-1-carbonyl)benzyl) phthalazin-1(2H)-one (75 mg, 0.17 mmol) and oxetane-2-carboxylic acid (92 mg, 0.38 mmol) in DMF (2 mL) was added EDCI (49 mg, 0.26 mmol), HOBt (35 mg, 0.26 mmol) and DIPEA (67 mg, 0.51 mmol). The reaction was stirred at 45° C. for 4 hours and purified by pre-HPLC to give N-(1-(5-((7-cyclobutoxy-4-oxo-3,4-dihydrophthalazin-1-yl)methyl)-2-fluorobenzoyl)azetidin-3-yl)-N-methyloxetane-2-carboxamide as a white solid (33 mg, 37% yield). LCMS ESI m/z 521 (M+H)⁺. ¹H-NMR (400 MHz, DMSO-d₆) δ 12.46 (s, 1H), 8.15 (d, J=8.8 Hz, 1H), 7.47 (d, J=22.8 Hz, 2H), 7.33-7.18 (m, 2H), 7.10 (s, 1H), 5.44-5.30 (m, 1H), 5.15-4.45 (m, 3H), 4.33 (d, J=19.1 Hz, 3H), 4.26-3.97 (m, 4H), 2.90 (d, J=25.3 Hz, 3H), 2.81-2.65 (m, 2H), 2.46-2.30 (m, 2H), 2.08-1.92 (m, 2H), 1.87-1.57 (m, 2H).

### Synthesis of Example 123: 2-((1-(5-((7-cyclobutoxy-4-oxo-3,4-dihydrophthalazin-1-yl)methyl)-2-fluorobenzoyl)azetidin-3-yl)(methyl)amino)-N-methylisonicotinamide

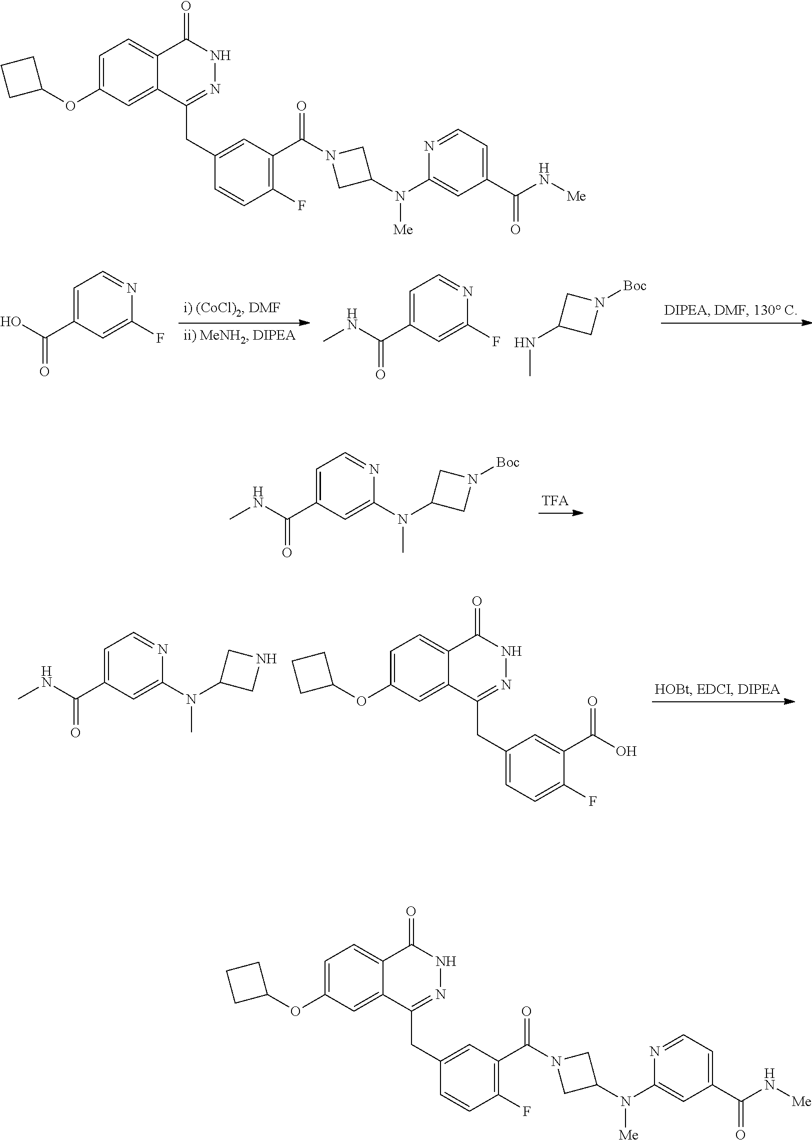

### Step 1:

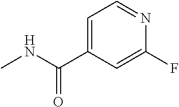

### 2-Fluoro-N-methylisonicotinamide

A solution of 2-fluoroisonicotinic acid (500 mg, 3.54 mmol), oxalyl chloride (450 mg, 387.19 mmol) and DMF (15 mg, 0.2 mmol) in anhydrous DCM (10 mL) was stirred at rt under Ar (g) for 30 mins. The reaction was concentrated under vacuum. The residue was dissolved in DCM (5 mL). This solution was added slowly to a mixture of DIPEA (1.37 g, 10.63 mmol) and methylamine hydrochloride (478 mg, 7.08 mmol) in DCM (10 mL) at 0° C. The reaction mixture was stirred at rt for 1 hour, then diluted with water (10 mL) and extracted with DCM (10 mL×3). The combined organic layer was dried over Na₂SO₄, filtered and concentrated in vacuo to give 2-fluoro-N-methylisonicotinamide as a yellow solid (331 mg, 61% yield). This was used without further purification. LCMS ESI m/z 155 [M+H]⁺.

### Step 2

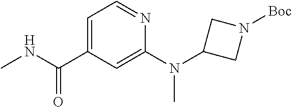 [SMILES: CNC(=O)c1ccnc(N(C)C2CN(C(=O)OC(C)(C)C)C2)c1]

### tert-Butyl 3-(methyl(4-(methylcarbamoyl)pyridin-2-yl)amino)azetidine-1-carboxylate

A mixture of 2-fluoro-N-methylisonicotinamide (331 mg, 1.64 mmol), tert-butyl 3-(methylamino)azetidine-1-carboxylate (1.2 g, 6.42 mmol) and DIPEA (693 mg, 5.35 mmol) in DMF (3 mL) was stirred at 130° C. for 16 hours. The mixture was cooled to rt, diluted with H₂O (5 mL) and extracted with EtOAc (10 mL×3). The combined organic layer was washed by brine, dried over Na₂SO₄, filtered and concentrated in vacuo. The residue was purified by silica gel chromatography (EtOAc/pet ether=30% to 45%) to obtain tert-butyl 3-(methyl(4-(methylcarbamoyl)pyridin-2-yl)amino)azetidine-1-carboxylate as a yellow oil (103 mg, 15% yield). LCMS ESI m/z 321 [M+H]⁺.

### Step 3:

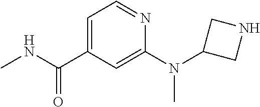 [SMILES: CNC(=O)c1ccnc(N(C)C2CNC2)c1]

### 2-(Azetidin-3-yl(methyl)amino)-N-methylisonicotinamide

A solution of tert-butyl 3-(methyl(4-(methylcarbamoyl)pyridin-2-yl)amino)azetidine-1-carboxylate (103 mg, 0.31 mmol) in DCM (3 mL) and TFA (0.3 mL) was stirred at rt for 1 hour. The solvent was removed under vacuum to afford 2-(azetidin-3-yl(methyl)amino)-N-methylisonicotinamide as a yellow oil (68 mg, 99% yield). This was used without further purification. LCMS ESI m/z 221 [M+H]⁺.

### Step 4

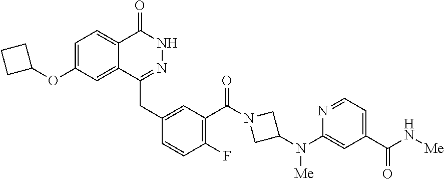 [SMILES: CNC(=O)c1ccnc(N(C)C2CN(C(=O)c3cc(Cc4n[nH]c(=O)c5ccc(OC6CCC6)cc45)ccc3F)C2)c1]

### 2-((1-(5-((7-Cyclobutoxy-4-oxo-3,4-dihydrophthalazin-1-yl)methyl)-2-fluorobenzoyl)azetidin-3-yl)(methyl)amino)-N-methylisonicotinamide

To a solution of 5-((7-cyclobutoxy-4-oxo-3,4-dihydrophthalazin-1-yl)methyl)-2-fluorobenzoic acid (60 mg, 0.16 mmol) and 2-(azetidin-3-yl(methyl)amino)-N-methylisonicotinamide (103 mg, 0.20 mmol) in DMF (2 mL) was added EDCI (47 mg, 0.24 mmol), HOBt (33 mg, 0.24 mmol) and DIPEA (63 mg, 0.49 mmol). The reaction was stirred at rt for 24 hours. The reaction was purified by prep-HPLC to give 2-((1-(5-((7-cyclobutoxy-4-oxo-3,4-dihydrophthalazin-1-yl)methyl)-2-fluorobenzoyl) azetidin-3-yl)(methyl)amino)-N-methylisonicotinamide as a white solid (29 mg, 30/o yield). LCMS ESI m/z: 571 (M+H)⁺. ¹H-NMR (400 MHz, DMSO-d₆) δ 12.51 (s, 1H), 9.42 (d, J=4.5 Hz, 1H), 8.78 (t, J=5.7 Hz, 1H), 8.44 (s, 1H), 8.33 (d, J=6.6 Hz, 1H), 8.15 (d, J=8.8 Hz, 1H), 7.60 (s, 1H), 7.47-7.27 (m, 3H), 7.24-7.06 (m, 3H), 4.90-4.74 (m, 2H), 4.67-4.54 (m, 1H), 4.49-4.39 (m, 1H), 4.34-3.94 (m, 5H), 3.83-3.72 (m, 1H), 3.66-3.54 (m, 1H), 2.78 (d, J=4.4 Hz, 3H), 2.46-2.34 (m, 2H), 2.09-1.94 (m, 2H), 1.87-1.58 (m, 2H).

### Synthesis of Example 124: 2-((1-(2-Fluoro-5-((7-(oxetan-3-yloxy)-4-oxo-3,4-dihydrophthalazin-1-yl)methyl)benzoyl)azetidin-3yl)(methyl)amino)isonicotinonitrile

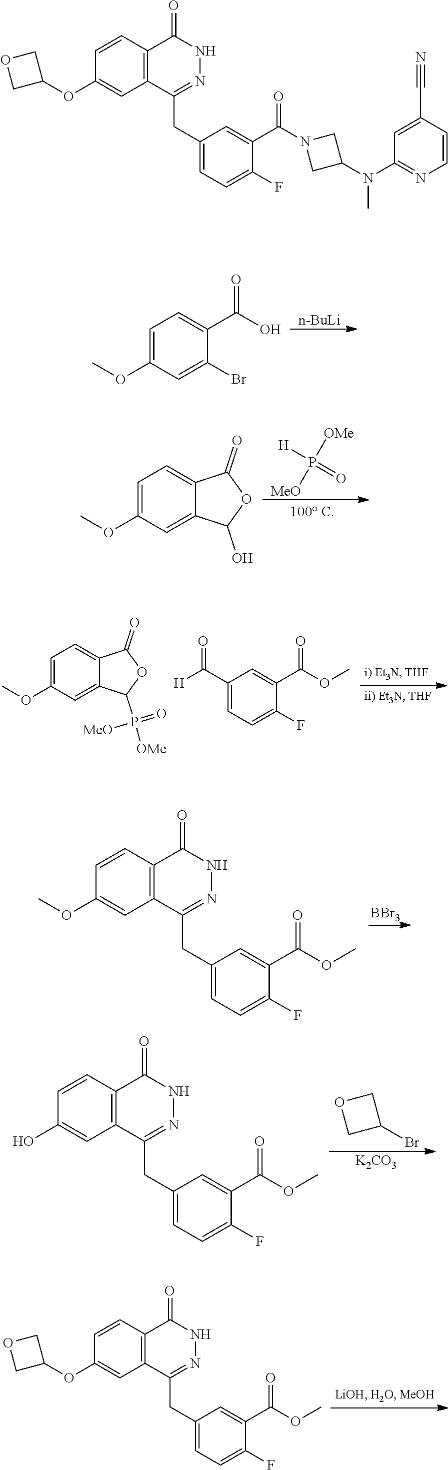

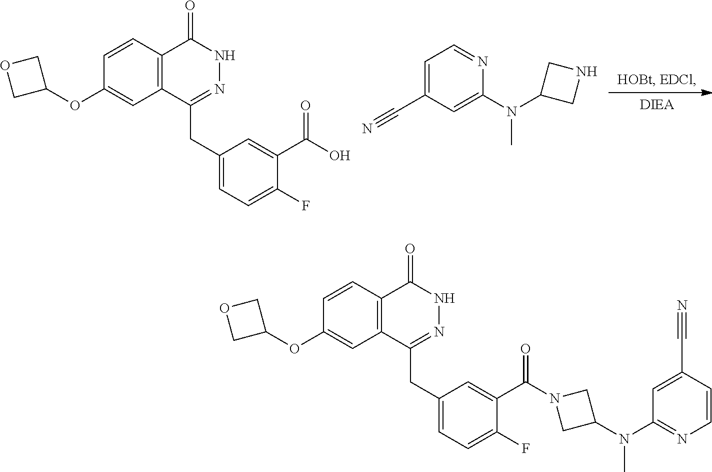

### Step 1

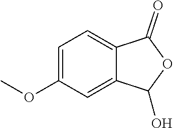

### 3-Hydroxy-5-methoxyisobenzofuran-1(3H)-one

A solution of 2-bromo-4-methoxybenzoic acid (4.98 g, 21.64 mmol) in THF (50 mL) was cooled to −78° C., then n-BuLi (2.5M in heptane, 17.31 mL, 43.28 mmol) was added dropwise under Ar (g). The reaction was stirred at −78° C. for 15 mins, then DMF (3.48 g, 47.61 mmol) was added dropwise. The reaction was kept at −78° C. for 20 mins, then quenched with sat. NH₄Cl solution (20 mL). The resulting mixture was added aq HCl (1N) until the solution reached pH 5˜6, then extracted with EtOAc (50×3 mL). The combined organic layer was washed with bine, dried over Na₂SO₄, filtered and concentrated in vacuo. The residue was purified by silica gel chromatography (EtOAc/pet ether=30 to 50%) to give 3-hydroxy-5-methoxyisobenzofuran-1(3H)-one as a white solid (1.5 g, 38% yield). LCMS ESI m/z 181 [M+H]⁺).

### Step 2

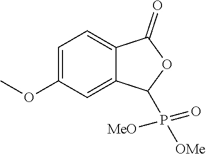 [SMILES: COc1ccc2c(c1)C(P(=O)(OC)OC)OC2=O]

### Dimethyl (6-methoxy-3-oxo-1,3-dihydroisobenzofuran-1-yl)phosphonate

A solution of 3-hydroxy-5-methoxyisobenzofuran-1(3H)-one (1.5 g, 8.30 mmol) in dimethylphosphite (30 mL) was heated to 100° C. and stirred for 2 hours. The mixture was cooled to rt, diluted with H₂O (10 mL) and extracted with EtOAc (30 mL×3). The combined organic layer was washed with brine, dried over anhydrous Na₂SO₄, filtered and concentrated in vacuo to give dimethyl (6-methoxy-3-oxo-1,3-dihydroisobenzofuran-1-yl)phosphonate as a light yellow oil (1.29 g, 57% yield). This was used without further purification. LCMS ESI m/z 273 [M+H]⁺.

### Step 3

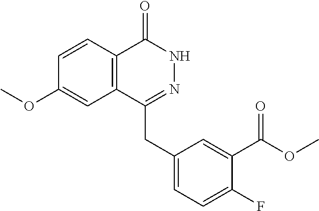

### Methyl 2-fluoro-5-((7-methoxy-4-oxo-3,4-dihydrophthalazin-1-yl)methyl)benzoate

A solution of dimethyl (6-methoxy-3-oxo-1,3-dihydroisobenzofuran-1-yl)phosphonate (1.29 g, 4.74 mmol), methyl 2-fluoro-5-formylbenzoate (863 mg, 4.74 mmol) and Et₃N (1.44 g, 14.22 mmol) in anhydrous THF (10 mL) was stirred at 60° C. under Ar(g) for 5 hours. Hydrazine hydrate (228 mg, 7.11 mmol) was added and the reaction was stirred at 70° C. for 1 hour. The reaction was concentrated under vacuum. The formed solids were washed with water (10 mL) and pet ether/EA (2:1, 10 mL) to give methyl 2-fluoro-5-((7-hydroxy-4-oxo-3,4-dihydrophthalazin-1-yl)methyl)benzoate as a white solid (1.15 g, 71% yield). This was used without further purification. LCMS ESI m/z 343 [M+H]⁺.

### Step 4

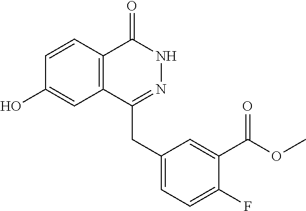 [SMILES: COC(=O)c1cc(Cc2n[nH]c(=O)c3ccc(O)cc23)ccc1F]

### Methyl 2-fluoro-5-((7-hydroxy-4-oxo-3,4-dihydrophthalazin-1-yl)methyl)benzoate

A solution of methyl 2-fluoro-5-((7-methoxy-4-oxo-3,4-dihydrophthalazin-1-yl)methyl)benzoate (800 mg, 1.46 mmol) and BBr₃ (30% in DCM, 12.17 g, 14.6 mmol) in DCM (40 mL) was stirred at rt for 16 hours. The mixture was diluted with H₂O (20 mL) and extracted with DCM (20 mL×3). The combined organic layer was dried over anhydrous Na₂SO₄, filtered and concentrated in vacuo to give methyl 2-fluoro-5-((7-hydroxy-4-oxo-3,4-dihydrophthalazin-1-yl)methyl)benzoate as a white solid (500 mg, 65% yield). This was used without further purification. LCMS ESI m/z 329 [M+H]⁺.

### Step 5

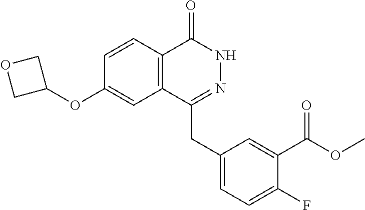 [SMILES: COC(=O)c1cc(Cc2n[nH]c(=O)c3ccc(OC4COC4)cc23)ccc1F]

### Methyl 2-fluoro-5-((7-(oxetan-3-yloxy)-4-oxo-3,4-dihydrophthalazin-1-yl)methyl)benzoate

A mixture of methyl 2-fluoro-5-((7-hydroxy-4-oxo-3,4-dihydrophthalazin-1-yl)methyl)benzoate (300 mg, 0.91 mmol), 3-bromooxetane (371 mg, 2.73 mmol) and K₂CO₃ (377 mg, 2.73 mmol) in DMF (3 mL) was stirred at 110° C. for 6 hours. The mixture was cooled to rt, poured into H₂O (5 mL), then aq HCl (1N) was added until the solution reached pH 5˜6. The mixture was extracted with EtOAc (20 mL×3). The combined organic layer was washed with brine, dried over Na₂SO₄, filtered and concentrated in vacuo to give methyl 2-fluoro-5-((7-(oxetan-3-yloxy)-4-oxo-3,4-dihydrophthalazin-1-yl)methyl)benzoate as a white solid (210 mg, 60% yield). This was used without further purification. LCMS ESI m/z 384 [M+H]⁺.

### Step 6

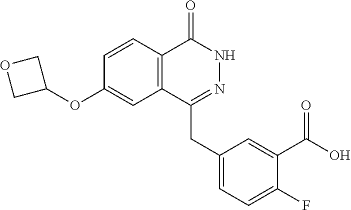 [SMILES: O=C(O)c1cc(Cc2n[nH]c(=O)c3ccc(OC4COC4)cc23)ccc1F]

### 2-Fluoro-5-((7-(oxetan-3-yloxy)-4-oxo-3,4-dihydrophthalazin-1-yl)methyl)benzoic acid

A mixture of methyl 2-fluoro-5-((7-(oxetan-3-yloxy)-4-oxo-3,4-dihydrophthalazin-1-yl)methyl)benzoate (200 mg, 0.52 mmol) and LiOH—H₂O (168 mg, 4 mmol) in MeOH (4 mL) and H₂O (2 mL) was stirred at rt for 3 hours. The organic solvent was removed in vacuo and aq HCl (1N) was added until the solution reached pH 5˜6. The solids were collected by filtration, washed with water (5 mL) and dried under vacuum to give 2-fluoro-5-((7-(oxetan-3-yloxy)-4-oxo-3,4-dihydrophthalazin-1-yl)methyl)benzoic acid as a white solid (123 mg, 64% yield). This was used without further purification. LCMS ESI m/z 370 [M+H]⁺.

### Step 7

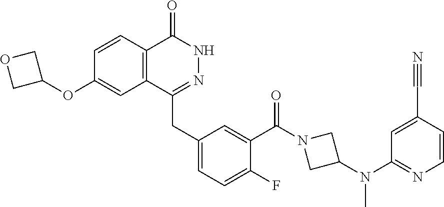 [SMILES: CN(c1cc(C#N)ccn1)C1CN(C(=O)c2cc(Cc3n[nH]c(=O)c4ccc(OC5COC5)cc34)ccc2F)C1]

### 2-((1-(2-Fluoro-5-((7-(oxetan-3-yloxy)-4-oxo-3,4-dihydrophthalazin-1-yl)methyl)benzoyl)azetidin-3-yl)methyl)amino)isonicotinonitrile

To a solution of 2-(azetidin-3-yl(methyl)amino)isonicotinonitrile (70 mg, 0.37 mmol) and 2-fluoro-5-((7-(oxetan-3-yloxy)-4-oxo-3,4-dihydrophthalazin-1-yl)methyl)benzoic acid (123 mg, 0.33 mmol) in DMF (2 mL) was added EDCI (117 mg, 0.61 mmol), HOBt (82 mg, 0.60 mmol) and DIPEA (158 mg, 1.22 mmol). The mixture was stirred at rt for 2 hours, then purified by prep-HPLC to give 2-((1-(2-fluoro-5-((7-(oxetan-3-yloxy)-4-oxo-3,4-dihydrophthalazin-1-yl)methyl)benzoyl)azetidin-3-yl)(methyl)amino)isonicotinonitrile as a yellow solid (46 mg, 65% yield). LCMS EST m/z 541 (M+H)⁺. ¹H NMR (400 MHz, DMSO-d₆) δ 12.51 (s, 1H), 8.59 (s, 1H), 8.44 (d, J=4 Hz, 1H), 8.19 (s, 1H), 7.96 (s, 1H), 7.44 (d, J=8 Hz, 1H), 7.31 (m, 1H), 7.26 (m, 1H), 7.23-7.15 (m, 1H), 7.00 (m, 1H), 5.47 (m, 1H), 4.93 (m, 2H), 4.87-4.79 (m, 1H), 4.56 (m, 1H), 4.49 (t, J=5.9 Hz, 3H), 4.27 (s, 2H), 3.65 (s, 1H), 3.62 (s, 3H).

### Synthesis of Example 125: 6-(4-(5-((7-Cyclobutoxy-4-oxo-3,4-dihydrophthalazin-1-yl)methyl)-2-fluorobenzoyl)-6,6-difluoro-1,4-diazepan-1-yl)nicotinonitrile

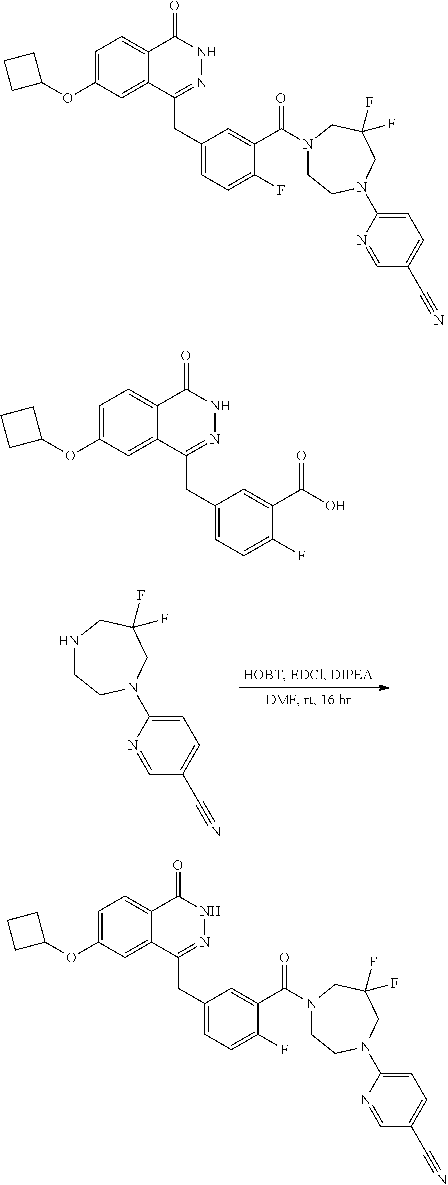 [SMILES: N#Cc1ccc(N2CCN(C(=O)c3cc(Cc4n[nH]c(=O)c5ccc(OC6CCC6)cc45)ccc3F)CC(F)(F)C2)nc1 ; O=C(O)c1cc(Cc2n[nH]c(=O)c3ccc(OC4CCC4)cc23)ccc1F ; N#Cc1ccc(N2CCNCC(F)(F)C2)nc1 ; N#Cc1ccc(N2CCN(C(=O)c3cc(Cc4n[nH]c(=O)c5ccc(OC6CCC6)cc45)ccc3F)CC(F)(F)C2)nc1]

### Step 1

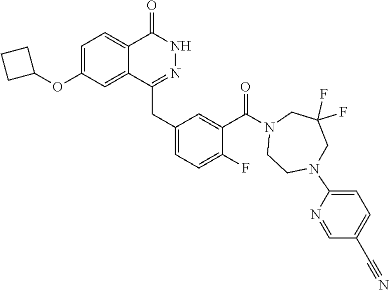 [SMILES: N#Cc1ccc(N2CCN(C(=O)c3cc(Cc4n[nH]c(=O)c5ccc(OC6CCC6)cc45)ccc3F)CC(F)(F)C2)nc1]

### 6-(4-(5-((7-Cyclobutoxy-4-oxo-3,4-dihydrophthalazin-1-yl)methyl)-2-fluorobenzoyl)-6,6-difluoro-1,4-diazepan-1-yl)nicotinonitrile

Following the amide coupling procedure in Example 126, but starting with methyl 5-((7-cyclobutoxy-4-oxo-3,4-dihydrophthalazin-1-yl)methyl)-2-fluorobenzoic acid and 6-(6,6-difluoro-1,4-diazepan-1-yl)nicotinonitrile gave the title compound as a white solid. LCMS ESI m/z: 589.2 [M+H]⁺. ¹H NMR (400 MHz, DMSO-d₆) δ 12.42 (d, J=5.2 Hz, 1H), 8.50 (dd, J=35.1, 2.0 Hz, 1H), 8.15 (dd, J=8.8, 4.2 Hz, 1H), 7.92 (dd, J=9.0, 2.3 Hz, 1H), 7.38 (dd, J=13.7, 11.1 Hz, 1H), 7.33-7.14 (m, 3H), 7.11-6.95 (m, 2H), 4.81 (m, 1H), 4.39-4.21 (m, 4H), 4.15 (s, 1H), 3.90 (m, 3H), 3.73 (s, 1H), 3.49 (t, J=5.3 Hz, 1H), 2.37 (s, 2H), 2.00 (t, J=9.6 Hz, 2H), 1.77 (m, 1H), 1.71-1.59 (m, 1H).

### Synthesis of Example 126: 6-Cyclobutoxy-4-(3-(6,6-difluoro-4-(5-(trifluoromethyl)pyrimidin-2-yl)-1,4-diazepane-1-carbonyl)-4-fluorobenzyl)phthalazin-1(2H)-one

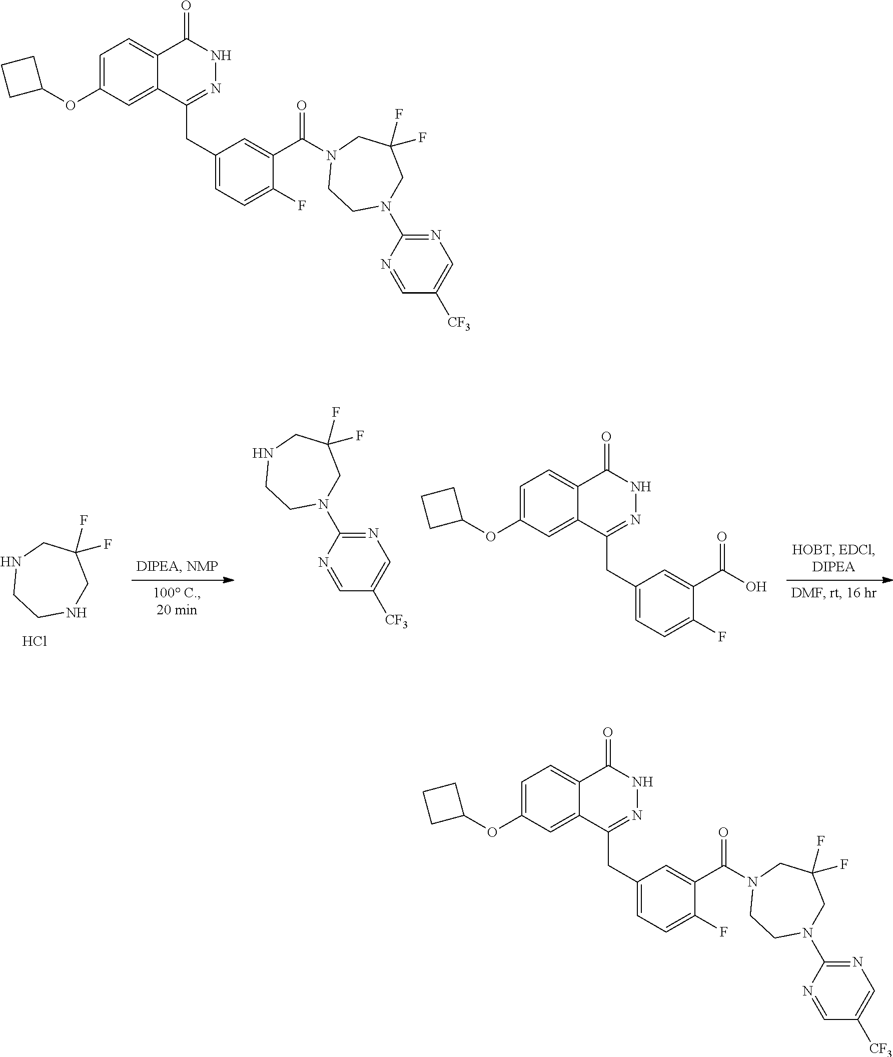 [SMILES: O=C(c1cc(Cc2n[nH]c(=O)c3ccc(OC4CCC4)cc23)ccc1F)N1CCN(c2ncc(C(F)(F)F)cn2)CC(F)(F)C1 ; FC1(F)CNCCNC1 ; FC1(F)CNCCN(c2ncc(C(F)(F)F)cn2)C1 ; O=C(O)c1cc(Cc2n[nH]c(=O)c3ccc(OC4CCC4)cc23)ccc1F ; Cl ; O=C(c1cc(Cc2n[nH]c(=O)c3ccc(OC4CCC4)cc23)ccc1F)N1CCN(c2ncc(C(F)(F)F)cn2)CC(F)(F)C1]

### Step 1

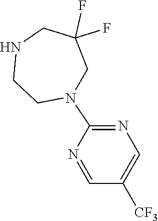 [SMILES: FC1(F)CNCCN(c2ncc(C(F)(F)F)cn2)C1]

### 6,6-Difluoro-1-(5-(trifluoromethyl)pyrimidin-2-yl)-1,4-diazepane

A solution of 6,6-difluoro-1,4-diazepane hydrochloride (27 mg, 156.43 μmol), 2-chloro-5-(trifluoromethyl)pyrimidine (28.55 mg, 156.43 μmol) and DIPEA (101.09 mg, 782.14 μmol) in NMP (1 mL) was stirred at 10) ° C. under microwave for 20 min. The reaction mixture was diluted in EtOAc, washed with water 3 times. The organic layer was concentrated to the crude and purified by Prep-TLC (EtOAc:Petroleum ether=3:1) to give 6,6-difluoro-1-[5-(trifluoromethyl)pyrimidin-2-yl]-1,4-diazepane (40.0 mg, 91% yield) as a white solid. LCMS ESI m/z: 282.9 [M+H]⁺.

### Step 2

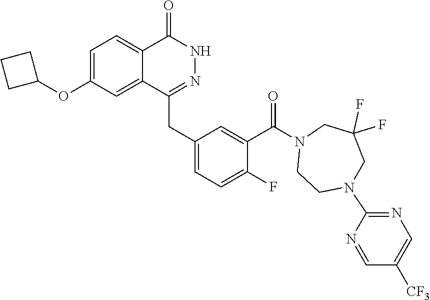 [SMILES: O=C(c1cc(Cc2n[nH]c(=O)c3ccc(OC4CCC4)cc23)ccc1F)N1CCN(c2ncc(C(F)(F)F)cn2)CC(F)(F)C1]

### 6-Cyclobutoxy-4-(3-(6,6-difluoro-4-(5-(trifluoromethyl)pyrimidin-2-yl)-1,4-diazepane-1-carbonyl)-4-fluorobenzyl)phthalazin-1(2H)-one

Following the amide coupling procedure above in Example 94, but starting with 6,6-difluoro-1-(5-(trifluoromethyl)pyrimidin-2-yl)-1,4-diazepane and 5-((7-cyclobutoxy-4-oxo-3,4-dihydrophthalazin-1-yl)methyl)-2-fluorobenzoic acid gave the title compound as a white solid. LCMS ESI m/z: 633.2 [M+H]⁺. ¹H NMR (400 MHz, DMSO-D6) a 12.42 (d, J=3.2 Hz, 1H), 8.75 (d, J=51.0 Hz, 2H), 8.15 (dd, J=8.8, 5.5 Hz, 1H), 7.45-7.34 (m, 1H), 7.34-7.12 (m, 3H), 7.06 (m, 1H), 4.80 (m, 1H), 4.42 (s, 2H), 4.32-4.04 (m, 4H), 3.94 (s, 2H), 3.77 (s, 1H), 3.51 (t, J=5.3 Hz, 1H), 2.36 (m, 2H), 2.07-1.91 (m, 2H), 1.76 (m, 1H), 1.65 (m, 1H).

### Synthesis of Example 127: 6-Cyclobutoxy-4-(3-(4-(cyclopropanecarbonyl)-6,6-difluoro-1,4-diazepane-1-carbonyl)-4-fluorobenzyl)phthalazin-1(2H)-one

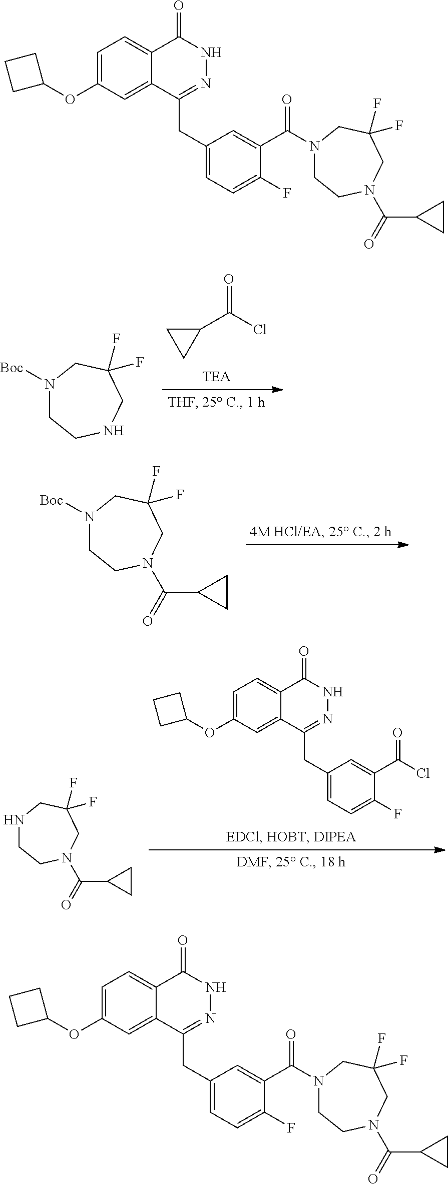

### Step 1

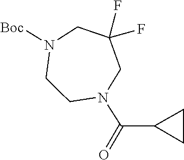 [SMILES: CC(C)(C)OC(=O)N1CCN(C(=O)C2CC2)CC(F)(F)C1]

### tert-butyl 4-(cyclopropanecarbonyl)-6,6-difluoro-1,4-diazepane-1-carboxylate

To a solution of tert-butyl 6,6-difluoro-1,4-diazepane-1-carboxylate (140 mg, 592.57 μmol) in THF (4 mL) was added Et₃N (179.89 mg, 1.78 mmol, 247.78 μL) and cyclopropanecarbonyl chloride (74.33 mg, 711.09 μmol, 64.64 μL) at 25° C. After stirring the reaction mixture 25° C. for 1 hr. NH₄Cl(aq.) and EtOAc were added in the mixture, which became two phases. The organic layer was separated, washed with aq. Na₂CO₃, dried over Na₂SO₄ and concentrated to give tert-butyl 4-(cyclopropanecarbonyl)-6,6-difluoro-1,4-diazepane-1-carboxylate (130 mg, 72% yield) as a yellow oil. ¹H NMR (400 MHz, CDCl₃) δ 3.99 (t, J=11.9 Hz, 2H), 3.88-3.51 (m, 6H), 1.48 (s, 9H), 1.20-1.14 (m, 1H), 1.05-0.99 (m, 2H), 0.83 (td. J=6.8, 3.8 Hz, 2H).

### Step 2

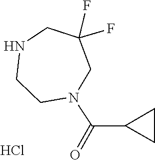 [SMILES: Cl ; O=C(C1CC1)N1CCNCC(F)(F)C1]

### cyclopropyl(6,6-difluoro-1,4-diazepan-1-yl)methanone hydrochloride

To a mixture of tert-butyl 4-(cyclopropanecarbonyl)-6,6-difluoro-1,4-diazepane-1-carboxylate (140 mg, 460.02 μmol) in 4M HCU/EA (5 mL). The reaction mixture was stirred at rt, for 2 hr. The mixture was concentrated to give cyclopropyl-(6,6-difluoro-1,4-diazepan-1-yl)methanone (110 mg, 457.04 μmol, 99.35% yield, HCl) as a white solid. LCMS ESI m/z: 205.2 [M+H]⁺.

### Step 3

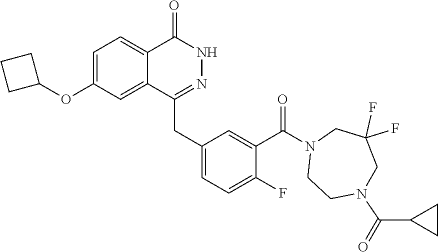 [SMILES: O=C(c1cc(Cc2n[nH]c(=O)c3ccc(OC4CCC4)cc23)ccc1F)N1CCN(C(=O)C2CC2)CC(F)(F)C1]

### 6-cyclobutoxy-4-(3-(4-(cyclopropanecarbonyl)-6,6-difluoro-1,4-diazepane-1-carbonyl)-4-fluorobenzyl)phthalazin-1(2H)-one

Following the amide coupling procedure above in Example 94, but starting with cyclopropyl(6,6-difluoro-1,4-diazepan-1-yl)methanone hydrochloride gave the title compound as a white solid. LCMS ESI m/z: 555.2 [M+H]⁺. ¹H NMR (400 MHz, DMSO-D6) δ 12.42 (d, J=2.4 Hz, 1H), 8.15 (dd, J=8.8, 3.2 Hz, 1H), 7.49-7.19 (m, 4H), 7.15-7.01 (m, 1H), 4.89-4.73 (m, 1H), 4.29 (s, 2H), 4.26-4.10 (m, 2H), 3.97 (m, 2H), 3.76 (m, 2H), 3.64-3.34 (m, 2H), 2.38 (s, 2H), 2.00 (d, J=8.0 Hz, 3H), 1.83-1.61 (m, 2H), 0.70 (m, 4H).

### Synthesis of Example 128: 6-cyclobutoxy-4-(4-fluoro-3-(4-(4-methoxypyridin-2-yl)piperazine-1-carbonyl)benzyl)phthalazin-1(2H)-one

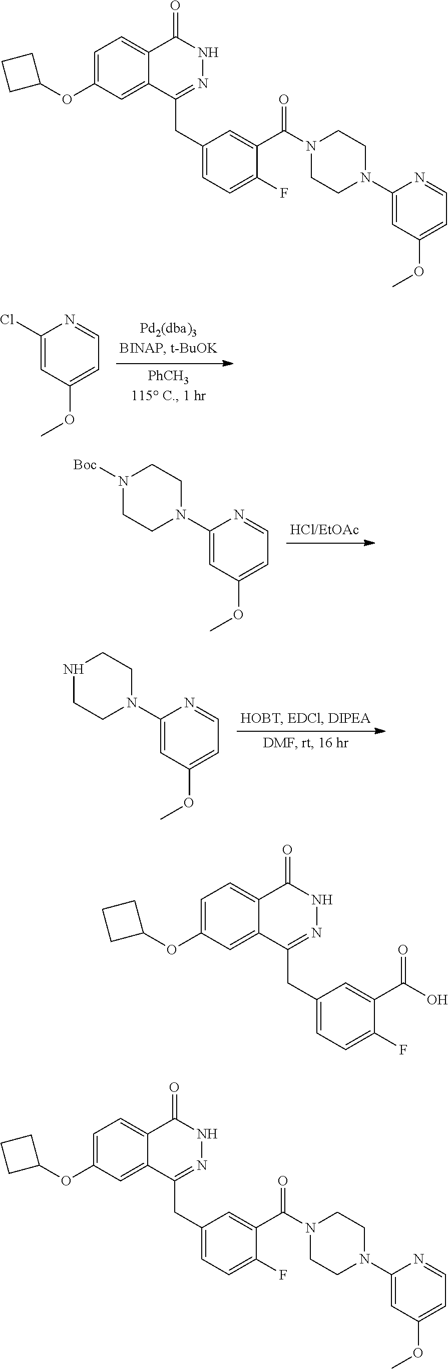

### Step 1

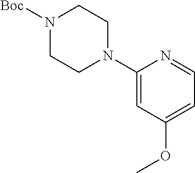 [SMILES: COc1ccnc(N2CCN(C(=O)OC(C)(C)C)CC2)c1]

### tert-butyl 4-(4-methoxypyridin-2-yl)piperazine-1-carboxylate

To a solution of 2-chloro-4-methoxy-pyridine (200 mg, 1.39 mmol) in PhCH₃ (4 mL) was added tert-butyl piperazine-1-carboxylate (778.37 mg, 4.18 mmol), Pd₂(dba)₃ (63.78 mg, 69.65 μmol), BINAP (86.74 mg, 139.30 μmol) and t-BuOK (468.95 mg, 4.18 mmol). The reaction mixture was stirred under microwave at 115° C. for 1 hr. The reaction mixture was filtered. The filtrate was poured into water (10 mL), extracted with EA (10 mL*2). The organic layer was washed with brine, dried and concentrated to the residue. The residue was purified by silica gel chromatography to give tert-butyl 4-(4-methoxy-2-pyridyl)piperazine-1-carboxylate (130 mg, 31.8% yield) as a light green oil. LCMS ESI m/z: 294.3 [M+H]⁺.

### Step 2 and 3

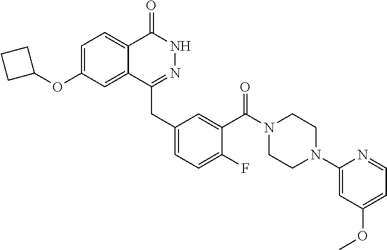 [SMILES: COc1ccnc(N2CCN(C(=O)c3cc(Cc4n[nH]c(=O)c5ccc(OC6CCC6)cc45)ccc3F)CC2)c1]

### 6-cyclobutoxy-4-(4-fluoro-3-(4-(4-methoxypyridin-2-yl)piperazine-1-carbonyl)benzyl)phthalazin-1(2H)-one

Following the amide coupling two-step procedure above in Example 94, but starting with tert-butyl 4-(4-methoxypyridin-2-yl)piperazine-1-carboxylate gave the title compound as a white solid. LCMS ESI m/z: 543.8 [M+H]⁺. ¹H NMR (400 MHz, DMSO-D6) δ 12.44 (s, 1H), 8.15 (t, J=6.1 Hz, 1H), 7.94 (d, 1=5.7 Hz, 1H), 7.41 (dd, J=6.3, 2.2 Hz, 2H), 7.34-7.19 (m, 2H), 7.11 (d, J=2.3 Hz, 1H), 6.36-6.27 (m, 2H), 4.89-4.77 (m, 1H), 4.31 (s, 2H), 3.78 (s, 3H), 3.71 (s, 2H), 3.57 (d, J=4.8 Hz, 2H), 3.42 (s, 2H), 3.27 (s, 2H), 2.45-2.34 (m, 2H), 2.08-1.94 (m, 2H), 1.72 (m, 2H).

### Synthesis of Example 129: 6-(4-(5-((7-cyclobutoxy-4-oxo-3,4-dihydrophthalazin-1-yl)methyl)-2-fluorobenzoyl)piperazin-1-yl)-5-(trifluoromethyl)nicotinonitrile

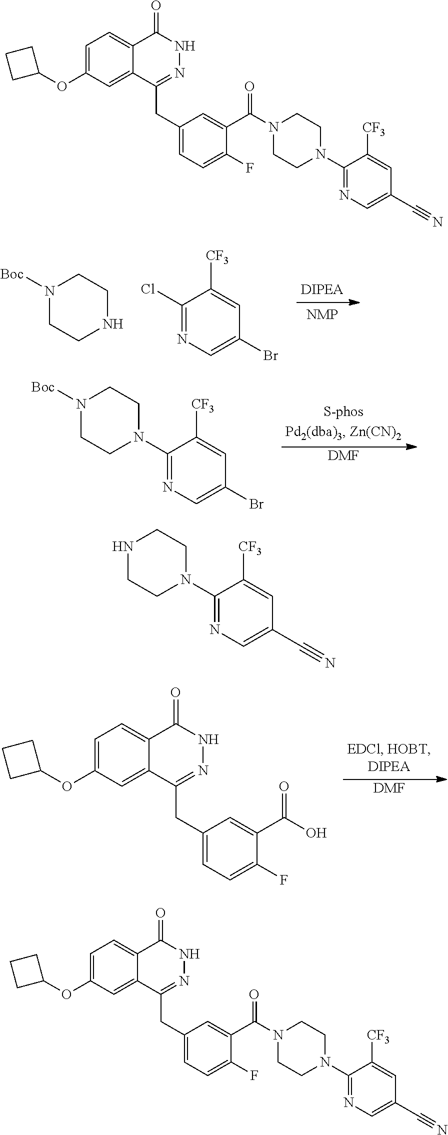

### Step 1,

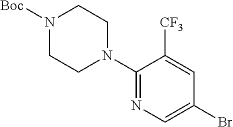 [SMILES: CC(C)(C)OC(=O)N1CCN(c2ncc(Br)cc2C(F)(F)F)CC1]

### tert-butyl 4-(5-bromo-3-(trifluoromethyl)pyridin-2-yl)piperazine-1-carboxylate

Following the general one-step procedure above in Example 23, but starting with 5-bromo-2-chloro-3-(trifluoromethyl)pyridine gave the title compound as yellow oil. LCMS ESI m/z: 356.0 [M+H-56]⁺.

### Step 2,

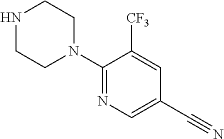 [SMILES: N#Cc1cnc(N2CCNCC2)c(C(F)(F)F)c1]

### 6-(piperazin-1-yl)-5-(trifluoromethyl)nicotinonitrile

A mixture of tert-butyl 4-[5-bromo-3-(trifluoromethyl)-2-pyridyl]piperazine-1-carboxylate (100 mg, 243.77 μmol), Zinc Cyanide (17.17 mg, 146.26 μmol, 9.27 μL) and Pd₂(dba)₃ (22.32 mg, 24.38 μmol) in DMF (1.5 mL) was stirred at 140° C. under N₂ for 60 min. The reaction was worked up with water and EtOAc, the organic layer was dried and concentrated. The crude product was used to the next step directly. LCMS ESI m/z: 257.2 [M+H]⁺.

### Step 3,

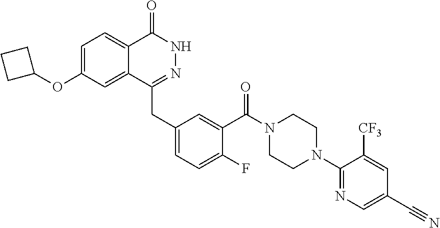 [SMILES: N#Cc1cnc(N2CCN(C(=O)c3cc(Cc4n[nH]c(=O)c5ccc(OC6CCC6)cc45)ccc3F)CC2)c(C(F)(F)F)c1]

### 6-(4-(5-((7-cyclobutoxy-4-oxo-3,4-dihydrophthalazin-1-yl)methyl)-2-fluorobenzoyl)piperazin-1-yl)-5-(trifluoromethyl)nicotinonitrile

Following the amide coupling procedure above in Example 94, but starting with 6-(piperazin-1-yl)-5-(trifluoromethyl)nicotinonitrile gave the title compound as a white solid. LCMS ESI m/z: 606.7 [M+H]⁺. ¹H NMR (400 MHz, DMSO-D6) δ 12.44 (s, 1H), 8.82 (d, J=2.0 Hz, 1H), 8.57 (d, J=2.0 Hz, 1H), 8.15 (d, J=8.8 Hz, 1H), 7.47-7.37 (m, 2H), 7.34-7.21 (m, 2H), 7.09 (d, J=2.3 Hz, 1H), 4.83 (p, J=7.1 Hz, 1H), 4.30 (s, 2H), 3.76 (s, 2H), 3.61 (s, 2H), 3.46 (s, 2H), 3.33 (s, 2H), 2.45-2.31 (m, 2H), 2.06-1.92 (m, 2H), 1.82-1.58 (m, 2H).

### Synthesis of Example 130: 6-cyclobutoxy-4-(4-fluoro-3-(4-(5-(hydroxymethyl)pyridin-2-yl)piperazine-1-carbonyl)benzyl)phthalazin-1(2H)-one

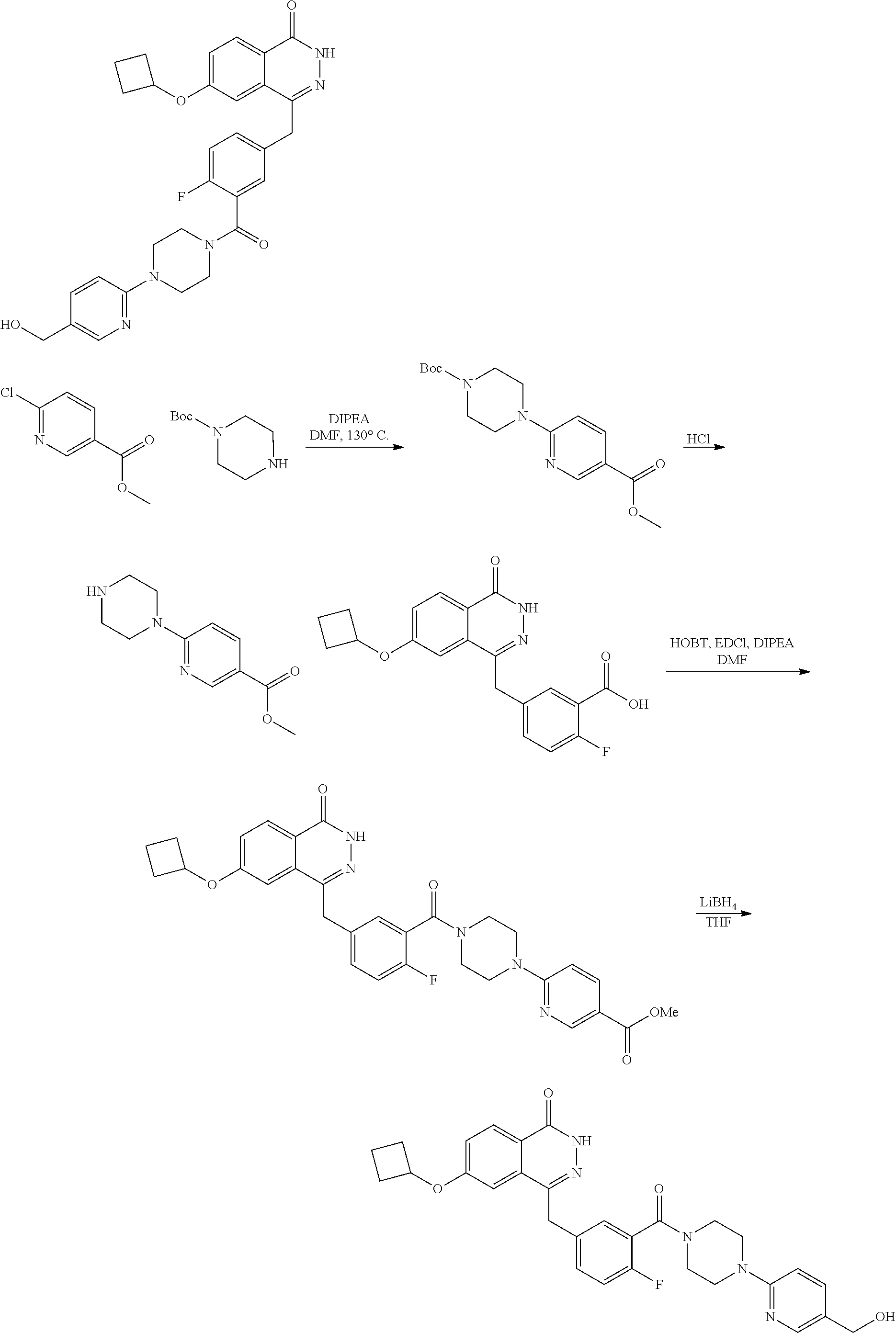

### Step 1

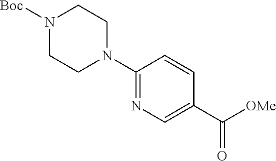 [SMILES: COC(=O)c1ccc(N2CCN(C(=O)OC(C)(C)C)CC2)nc1]

### tert-butyl 4-(5-(methoxycarbonyl)pyridin-2-yl)piperazine-1-carboxylate

To a solution of methyl 6-chloronicotinate (500 mg, 2.91 mmol) and tert-butyl piperazine-1-carboxylate (543 mg, 2.91 mmol) in DMF (5 mL) was added DIPEA (1.51 g, 11.66 mmol). The reaction mixture was stirred at 130° C. for 3 hours, then quenched with water (30 mL) and extracted with EtOAc (30 mL×3). The combined organic layer was washed with brine, dried over anhydrous Na₂SO₄, filtered and concentrated in vacuo. The residue was purified by silica gel chromatography (EtOAc/pet ether=10% to 40%) to give tert-butyl 4-(5-(methoxycarbonyl)pyridin-2-yl)piperazine-1-carboxylate (780 mg, 83% yield) as a white solid. LCMS ESI m/z: 322 [M+H]⁺.

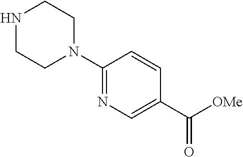 [SMILES: COC(=O)c1ccc(N2CCNCC2)nc1]

### methyl 6-(piperazin-1-yl)nicotinate

A solution of tert-butyl 4-(5-(methoxycarbonyl)pyridin-2-yl)piperazine-1-carboxylate (780 mg, 2.43 mmol) in HCl/Dioxane (4M, 10 mL) was stirred at rt for 1 hour. The solvent was removed in vacuo to give methyl 6-(piperazin-1-yl)nicotinate (510 mg, 95% yield) as a white solid. This was used without further purification. LCMS ESI m/z: 222 [M+H]⁺.

### Step 3

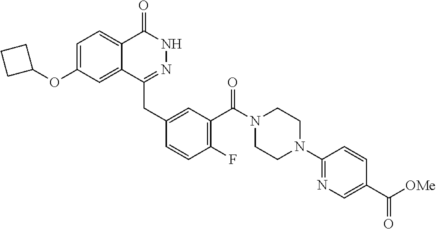 [SMILES: COC(=O)c1ccc(N2CCN(C(=O)c3cc(Cc4n[nH]c(=O)c5ccc(OC6CCC6)cc45)ccc3F)CC2)nc1]

### methyl 6-(4-(5-((7-cyclobutoxy-4-oxo-dihydrophthalazin-1-yl)methyl)-2-fluorobenzoyl)piperazin-1-yl)nicotinate

To a solution of 5-((7-cyclobutoxy-4-oxo-3,4-dihydrophthalazin-1-yl)methyl)-2-fluorobenzoic acid (1100 mg, 0.27 mmol) and methyl 6-(piperazin-1-yl)nicotinate (96 mg, 0.30 mmol) in DMF (2 mL) was added EDCI (78 mg, 0.41 mmol). HOBt (55 mg, 0.41 mmol) and DIPEA (175 mg, 1.36 mmol). The reaction was stirred at it for 4 hours, then quenched with water (20 mL) and extracted with EtOAc (20 mL×3). The combined organic layer was washed with brine, dried over anhydrous Na₂SO₄, filtered and concentrated in vacuo. The residue was purified by silica gel chromatography (EtOAc/pet ether=10% to 70%) to give methyl 6-(4-(5-((7-cyclobutoxy-4-oxo-3,4-dihydrophthalazin-1-yl)methyl)-2-fluorobenzoyl)piperazin-1-yl)nicotinate (130 mg, 84% yield) as a white solid. LCMS ESI m/z: 572 [M+H]⁺.

### Step 4

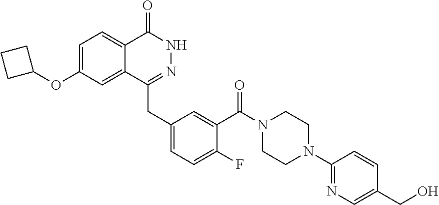 [SMILES: O=C(c1cc(Cc2n[nH]c(=O)c3ccc(OC4CCC4)cc23)ccc1F)N1CCN(c2ccc(CO)cn2)CC1]

### 6-cyclobutoxy-4-(4-fluoro-3-(4-(5-(hydroxymethyl)pyridin-2-yl)piperazine-1-carbonyl)benzyl)phthalazin-1(2H)-one

To a solution of methyl 6-(4-(5-((7-cyclobutoxy-4-oxo-3,4-dihydrophthalazin-1-yl)methyl)-2-fluorobenzoyl)piperazin-1-yl)nicotinate (130 mg, 0.23 mmol) in THF (5 mL) was added LiBH₄ (15 mg, 0.68 mmol). The reaction mixture was stirred at rt for 72 hours. The reaction was purified by prep-HPLC to give 6-cyclobutoxy-4-(4-fluoro-3-(4-(5-(hydroxymethyl)pyridin-2-yl)piperazine-1-carbonyl)benzyl)phthalazin-1(2H)-one (36 mg, 29%) as a white solid. LCMS ESI m/z: 544 [M+H]⁺. ¹H-NMR (400 MHz, CDCl₃) δ 10.83 (s, 1H), 8.35 (d, J=8.8 Hz, 1H), 8.17-8.14 (m, 1H), 7.57 (dd, J=8.7, 2.3 Hz, 1H), 7.37-7.34 (m, 1H), 7.34-7.29 (m, 1H), 7.20 (dd, J=8.8, 2.3 Hz, 1H), 7.06 (t, J=8.8 Hz, 1H), 6.91 (d, J=2.3 Hz, 1H), 6.66 (d, J=8.7 Hz, 1H), 4.71-4.63 (m, 1H), 4.57 (s, 2H), 4.23 (s, 2H), 3.93-3.84 (m, 2H), 3.65-3.60 (m, 2H), 3.56-3.51 (m, 2H), 3.45-3.36 (m, 2H), 2.45-2.38 (m, 2H), 2.20-2.11 (m, 2H), 1.93-1.85 (m, 1H), 1.81-1.71 (m, 1H).

### Synthesis of Example 131: 6-(4-(5-((7-cyclobutoxy-4-oxo-3,4-dihydrophthalazin-1-yl)methyl)-2-fluorobenzoyl)piperazin-1-yl)-5-methoxynicotinonitrile

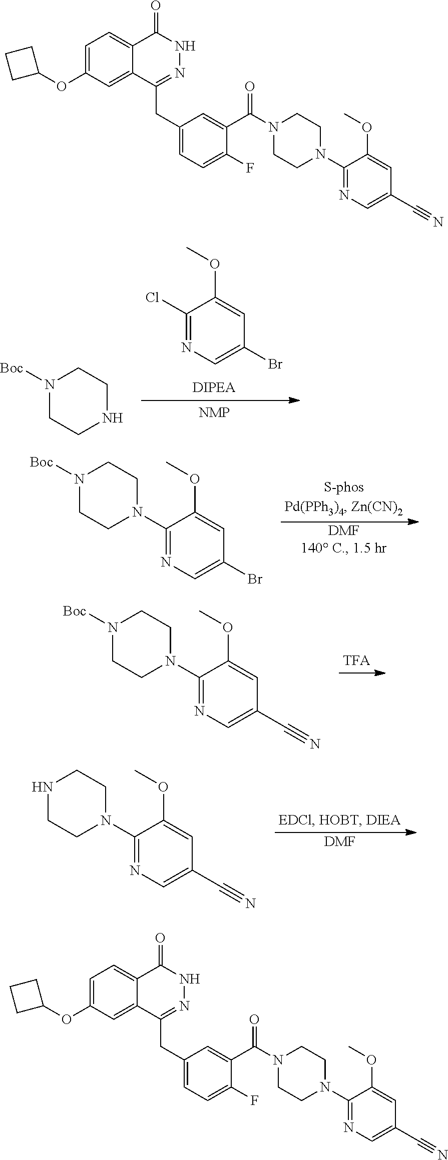

### Step 1

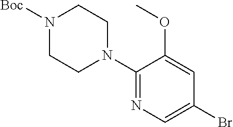 [SMILES: COc1cc(Br)cnc1N1CCN(C(=O)OC(C)(C)C)CC1]

### tert-butyl 4-(5-bromo-3-methoxypyridin-2-yl)piperazine-1-carboxylate

Following the general one-step procedure above in Example 130, but starting with 5-bromo-2-chloro-3-methoxypyridine gave the title compound as yellow oil. LCMS EST m/z: 374.1 [M+H]⁺.

### Step 2

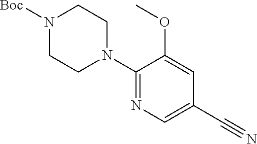 [SMILES: COc1cc(C#N)cnc1N1CCN(C(=O)OC(C)(C)C)CC1]

### tert-butyl 4-(5-cyano-3-methoxypyridin-2-yl)piperazine-1-carboxylate

To a solution of tert-butyl 4-(5-bromo-3-methoxy-2-pyridyl)piperazine-1-carboxylate (100 mg, 268.63 μmol) in DMF (2 mL) was added Zn(CN)₂ (31.54 mg, 268.63 μmol), Pd₂(dba)₃ (24.60 mg, 26.86 μmol), S-phos (11.03 mg, 26.86 μmol), and then the sealed tube was irradiated in microwave at 140° C. for 1.5 hr. The reaction was poured into H₂O (10 mL), extracted with EA (10 mL). The organic layer was washed with brine, dried and concentrated. The residue was purified by Prep-TLC to give tert-butyl 4-(5-cyano-3-methoxy-2-pyridyl)piperazine-1-carboxylate (40 mg, 46.7% yield) as a yellow solid. LCMS ESI m/z: 262.9 [M+H-56]⁺.

### Step 3, and 4

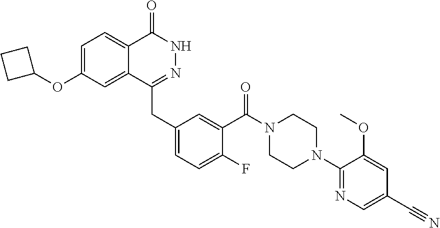 [SMILES: COc1cc(C#N)cnc1N1CCN(C(=O)c2cc(Cc3n[nH]c(=O)c4ccc(OC5CCC5)cc34)ccc2F)CC1]

### 6-(4-(5-((7-cyclobutoxy-4-oxo-3,4-dihydrophthalazin-1-yl)methyl)-2-fluorobenzoyl)piperazin-1-yl)-5-methoxynicotinonitrile

Following the deprotection and amide coupling two-step procedure above in Example 94, but starting with tert-butyl 4-(5-cyano-3-methoxypyridin-2-yl)piperazine-1-carboxylate gave the title compound as a white solid. LCMS ESI m/z: 568.8 [M+H]⁺. ¹H NMR (400 MHz, DMSO-D6) δ 12.44 (s, 1H), 8.21 (d, J=1.7 Hz, 1H), 8.15 (d, J=8.8 Hz, 1H), 7.61 (d, J=1.7 Hz, 1H), 7.39 (d, J=6.4 Hz, 2H), 7.33-7.21 (m, 2H), 7.08 (d, J=2.2 Hz, 1H), 4.86-4.78 (m, 1H), 4.30 (s, 2H), 3.84 (s, 3H), 3.72 (s, 2H), 3.64 (s, 2H), 3.48 (s, 2H), 3.28 (s, 2H), 2.44-2.34 (m, 2H), 1.99 (t, J=10.6 Hz, 2H), 1.77 (d, J=10.1 Hz, 1H), 1.65 (dd, J=19.0, 8.7 Hz, 1H).

### Synthesis of Example 132: 6-(4-(5-((7-cyclobutoxy-4-oxo-3,4-dihydrophthalazin-1-yl)methyl)-2-fluorobenzoyl)piperazin-1-yl)-4-(trifluoromethyl)nicotinonitrile

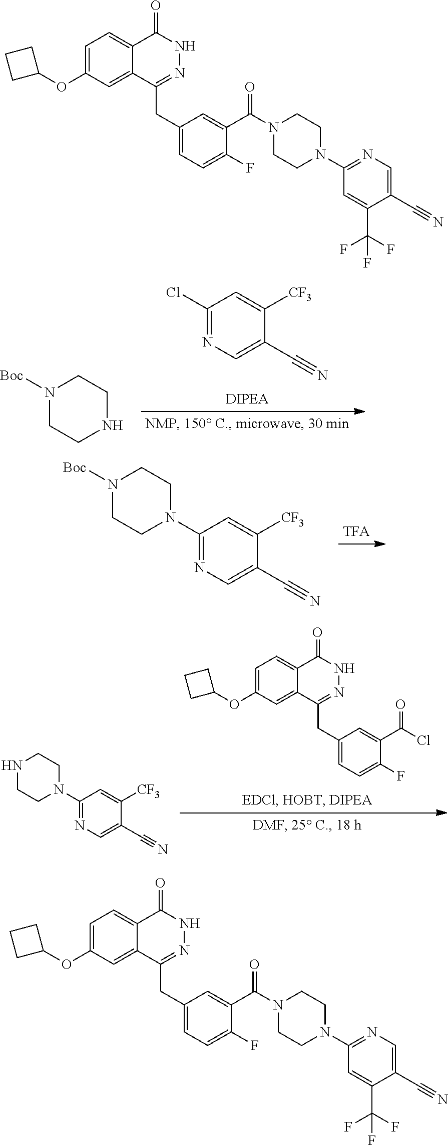

### Step 1, 2 and 3

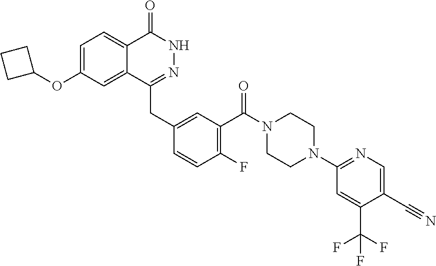 [SMILES: N#Cc1cnc(N2CCN(C(=O)c3cc(Cc4n[nH]c(=O)c5ccc(OC6CCC6)cc45)ccc3F)CC2)cc1C(F)(F)F]

### 6-(4-(5-((7-cyclobutoxy-4-oxo-3,4-dihydrophthalazin-1-yl)methyl)-2-fluorobenzoyl)piperazin-1-yl)-4-(trifluoromethyl)nicotinonitrile

Following the general three-step procedure above in Example 130, but starting with 6-chloro-4-(trifluoromethyl)nicotinonitrile gave the title compound as a white solid. LCMS ESI m/z: 606.6 [M+H]⁺. ¹H NMR (400 MHz, DMSO-D6) δ 12.44 (s, 1H), 8.73 (s, 1H), 8.16 (d, J=8.8 Hz, 1H), 7.43 (t, J=9.0 Hz, 2H), 7.35-7.22 (m, 3H), 7.10 (d, J=2.2 Hz, 1H), 4.88-4.79 (m, 1H), 4.31 (s, 2H), 3.88 (s, 2H), 3.74 (s, 4H), 3.36 (s, 2H), 2.40 (m, 2H), 2.06-1.96 (m, 2H), 1.82-1.63 (m, 2H).

### Synthesis of Example 133: 6-(4-(5-((7-cyclobutoxy-4-oxo-3,4-dihydrophthalazin-1-yl)methyl)-2-fluorobenzoyl)piperazin-1-yl)-2-methylnicotinonitrile

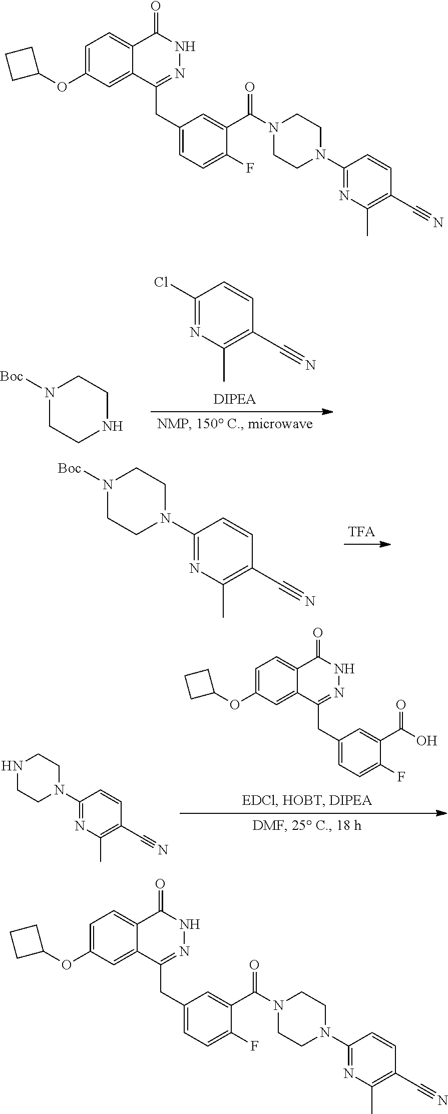

### Step 1, 2 and 3

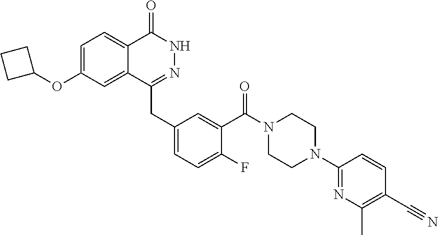 [SMILES: Cc1nc(N2CCN(C(=O)c3cc(Cc4n[nH]c(=O)c5ccc(OC6CCC6)cc45)ccc3F)CC2)ccc1C#N]

### 6-(4-(5-((7-cyclobutoxy-4-oxo-3,4-dihydrophthalazin-1-yl)methyl)-2-fluorobenzoyl)piperazin-1-yl)-2-methylnicotinonitrile

Following the general three-step procedure above in Example 130, but starting with 6-chloro-2-methylnicotinonitrile gave the tide compound as a white solid. LCMS ESI m/z: 552.7 [M+H]⁺. ¹H NMR (400 MHz, DMSO-D6) δ 12.44 (s, 1H), 8.16 (d, J=8.7 Hz, 1H), 7.82 (d, J=8.9 Hz, 1H), 7.40 (d, J=6.1 Hz, 2H), 7.35-7.19 (m, 2H), 7.09 (s, 1H), 6.75 (m, 1H), 4.86-4.79 (m, 1H), 4.31 (s, 2H), 3.73 (d, J=12.6 Hz, 4H), 3.58 (s, 2H), 3.29-3.23 (m, 2H), 2.47 (s, 3H), 2.39 (m, 2H), 2.05-1.95 (m, 2H), 1.81-1.63 (m, 2H).

### Synthesis of Example 134: 6-(4-(5-((7-cyclobutoxy-4-oxo-3,4-dihydrophthalazin-1-yl)methyl)-2-fluorobenzoyl)piperazin-1-yl)-4-methylnicotinonitrile

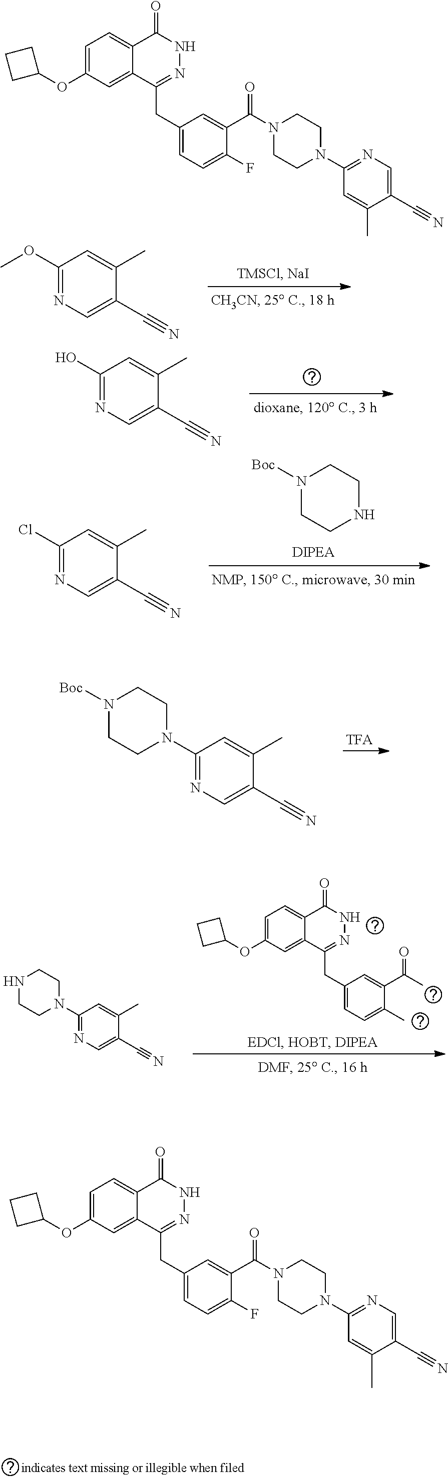

### Step 1

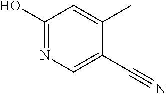

### 6-hydroxy-4-methylnicotinonitrile

To a solution of 6-methoxy-4-methyl-pyridine-3-carbonitrile (200 mg, 1.35 mmol) in CH₃CN (3 mL) was added Chlorotrimethylsilane (293.30 mg, 2.70 mmol, 342.64 μL) and NaI (404.67 mg, 2.70 mmol, 110.35 μL). The reaction mixture was stirred at rt, for 18 hr. Water and EtOAc were added in the mixture, which became two phases. The organic layer was separated, washed with NaCl (aq), dried over Na₂SO₄ and concentrated to give a residue. The residue was purified by silica gel chromatography (petroleum ether/EtOAc=20/1 to 1/1) to give 6-hydroxy-4-methyl-pyridine-3-carbonitrile (76 mg, 41.9% yield) as a yellow solid. ¹H NMR (400 MHz, DMSO-D6) δ 12.26 (s, 1H), 8.20 (s, 1H), 6.32 (s, 1H), 2.22 (d, J=0.9 Hz, 3H).

### Step 2

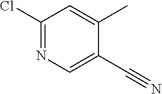

### 6-chloro-4-methylnicotinonitrile

To a solution of 6-hydroxy-4-methyl-pyridine-3-carbonitrile (76 mg, 566.59 μmol) in dioxane (3 mL) was added POCl₃ (130.32 mg, 849.89 μmol) at 25° C. under N₂. The reaction mixture was stirred at 120° C. for 3 hr. The reaction mixture was poured into ice-cold water, extracted with EA. The organic layer was washed with NaCl (aq), dried over Na₂SO₄ and concentrated to give a residue. The residue was purified by silica gel chromatography (petroleum ether/EtOAc=20/1 to 1/1) to give 6-chloro-4-methylnicotinonitrile (54 mg, 62.4% yield) as a yellow solid. ¹H NMR (400 MHz, DMSO-D6) δ 8.82 (s, 1H), 7.75 (s, 1H), 2.51 (s, 3H).

### Step 3, 4 and 5

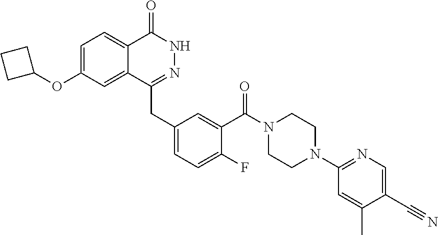 [SMILES: Cc1cc(N2CCN(C(=O)c3cc(Cc4n[nH]c(=O)c5ccc(OC6CCC6)cc45)ccc3F)CC2)ncc1C#N]

### 6-(4-(5-((7-cyclobutoxy-4-oxo-3,4-dihydrophthalazin-1-yl)methyl)-2-fluorobenzoyl)piperazin-1-yl)-4-methylnicotinonitrile

Following the general three-step procedure above in Example 130, but starting with 6-chloro-4-methylnicotinonitrile gave the title compound as a white solid. LCMS ESI m/z: 552.7 [M+H]⁺. ¹H NMR (400 MHz, DMSO-D6) δ 12.44 (s, 1H), 8.42 (s, 1H), 8.16 (d, J=8.8 Hz, 1H), 7.42 (t, J=8.0 Hz, 2H), 7.34-7.23 (m, 2H), 7.10 (d, J=2.1 Hz, 1H), 6.86 (s, 1H), 4.83 (m, 1H), 4.30 (s, 2H), 3.73 (d, J=6.8 Hz, 4H), 3.60 (s, 2H), 3.30-3.26 (m, 2H), 2.39 (s, 2H), 2.35 (s, 3H), 2.06-1.95 (m, 2H), 1.80-1.63 (m, 2H).

### Synthesis of Example 135: 6-(4-(5-((7-cyclobutoxy-4-oxo-3,4-dihydrophthalazin-1-yl)methyl)-2-fluorobenzoyl)piperazin-1-yl)-2-(trifluoromethyl)nicotinonitrile

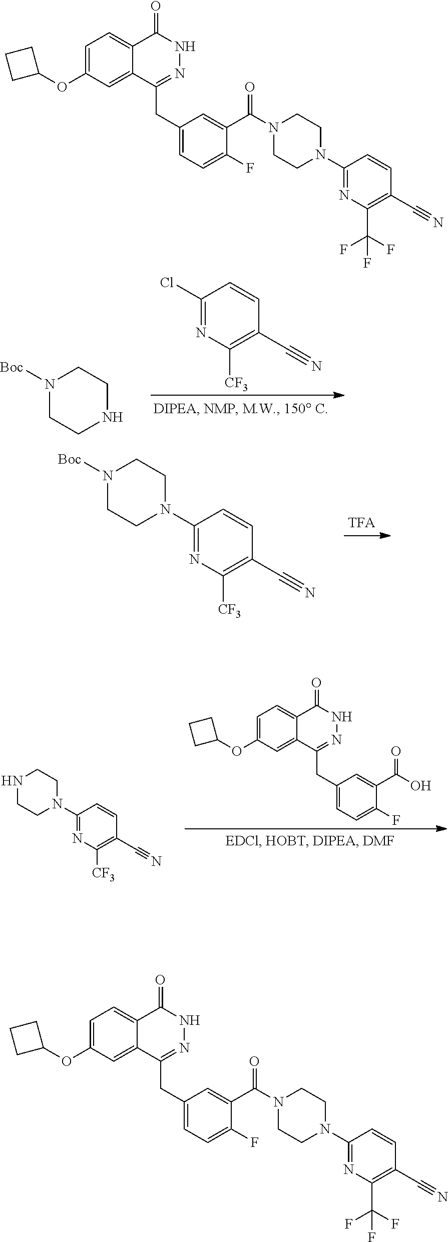

### Step 1, 2 and 3

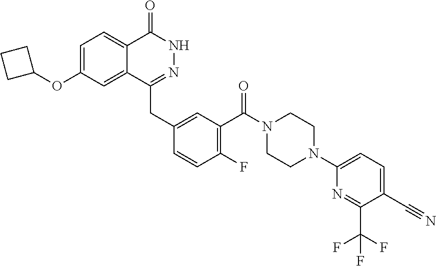 [SMILES: N#Cc1ccc(N2CCN(C(=O)c3cc(Cc4n[nH]c(=O)c5ccc(OC6CCC6)cc45)ccc3F)CC2)nc1C(F)(F)F]

### 6-(4-(5-((7-cyclobutoxy-4-oxo-3,4-dihydrophthalazin-1-yl)methyl)-2-fluorobenzoyl)piperazin-1-yl)-2-(trifluoromethyl)nicotinonitrile

Following the general three-step procedure above in Example 130, but starting with 6-chloro-2-(trifluoromethyl)nicotinonitrile gave the title compound as a white solid. LCMS ESI in/z: 606.7 [M+H]⁺. ¹H NMR (400 MHz, DMSO-D6) δ 12.44 (s, 1H), 8.15 (dd, J=9.0, 4.0 Hz, 2H), 7.46-7.38 (m, 2H), 7.34-7.25 (m, 2H), 7.19 (d, J=9.2 Hz, 1H), 7.09 (d, J=2.2 Hz, 1H), 4.88-4.80 (m, 1H), 4.31 (s, 2H), 3.85-3.63 (m, 6H), 3.33 (s, 2H), 2.43-2.34 (m, 2H), 2.06-1.96 (m, 2H), 1.70 (m, 2H).

### Synthesis of Example 136: 6-(4-(5-((7-cyclobutoxy-4-oxo-3,4-dihydrophthalazin-1-yl)methyl)-2-fluorobenzoyl)piperazin-1-yl)-2-methoxynicotinonitrile

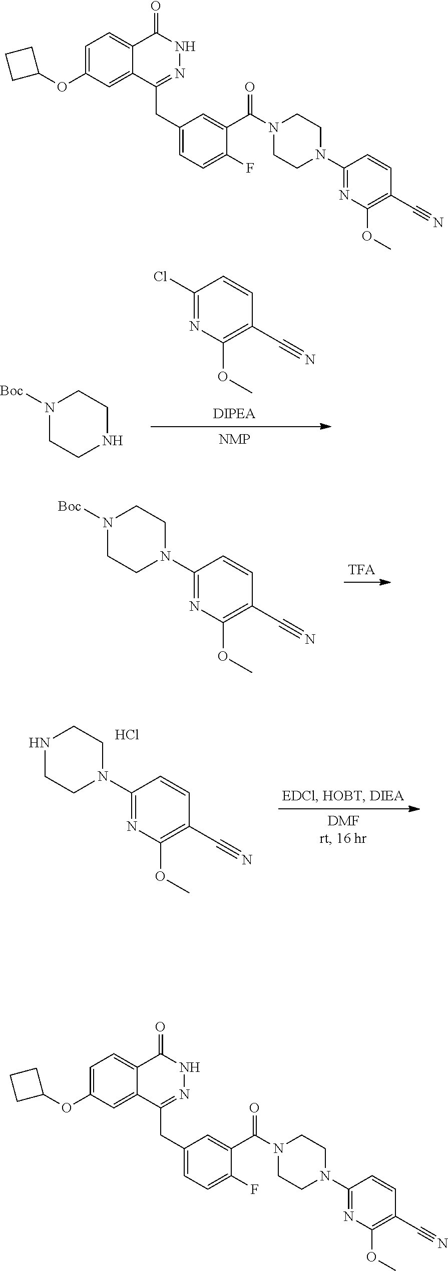

### Step 1, 2 and 3

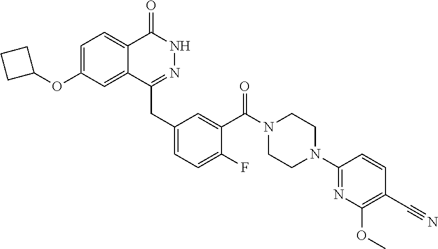 [SMILES: COc1nc(N2CCN(C(=O)c3cc(Cc4n[nH]c(=O)c5ccc(OC6CCC6)cc45)ccc3F)CC2)ccc1C#N]

### 6-(4-(5-((7-cyclobutoxy-4-oxo-3,4-dihydrophthalazin-1-yl)methyl)-2-fluorobenzoyl)piperazin-1-yl)-2-methoxynicotinonitrile

Following the general three-step procedure above in Example 130, but starting with 6-chloro-2-methoxynicotinonitrile gave the title compound as a white solid. LCMS ESI m/z: 568.8 [M+H]⁺. ¹H NMR (400 MHz, DMSO-D6) δ 12.44 (s, 1H), 8.15 (t, J=6.9 Hz, 1H), 7.81 (dd, J=8.6, 4.5 Hz, 1H), 7.48-7.37 (m, 2H), 7.35-7.22 (m, 2H), 7.09 (d, J=2.3 Hz, 1H), 6.46 (d, J=8.7 Hz, 1H), 4.87-4.78 (m, 1H), 4.31 (s, 2H), 3.90 (s, 3H), 3.74 (d, J=8.8 Hz, 4H), 3.59 (s, 2H), 3.30-3.25 (m, 2H), 2.43-2.33 (m, 2H), 2.06-1.94 (m, 2H), 1.83-1.60 (m, 2H).

### Synthesis of Example 137: 6-(4-(5-((7-cyclobutoxy-4-oxo-3,4-dihydrophthalazin-1-yl)methyl)-2-fluorobenzoyl)piperazin-1-yl)-4-methoxynicotinonitrile

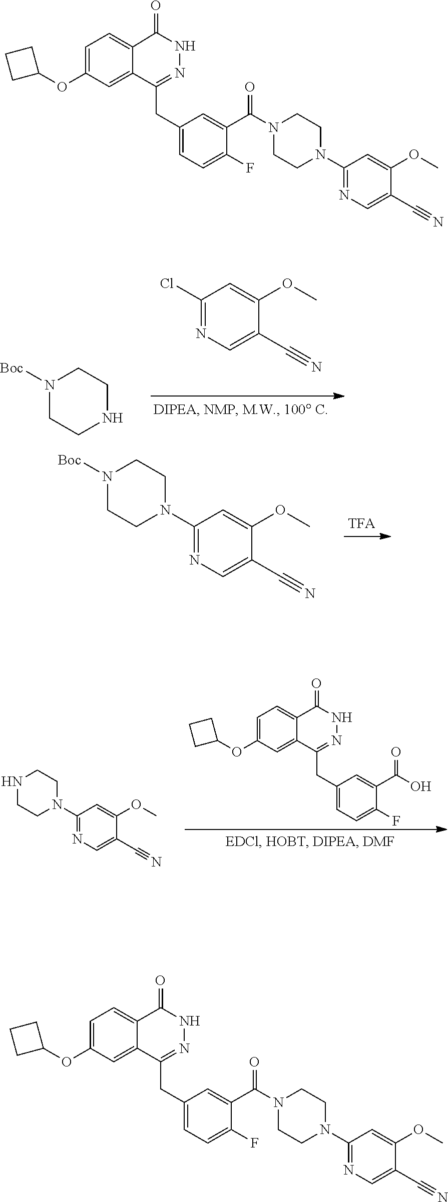

### Step 1, 2 and 3

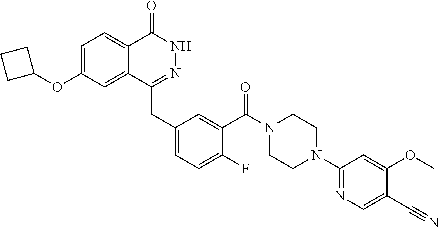 [SMILES: COc1cc(N2CCN(C(=O)c3cc(Cc4n[nH]c(=O)c5ccc(OC6CCC6)cc45)ccc3F)CC2)ncc1C#N]

### 6-(4-(5-((7-cyclobutoxy-4-oxo-3,4-dihydrophthalazin-1-yl)methyl)-2-fluorobenzoyl)piperazin-1-yl)-4-methoxynicotinonitrile

Following the general three-step procedure above in Example 130, but starting with 6-chloro-4-methoxynicotinonitrile gave the title compound as a white solid. LCMS ESI m/z: 568.8 [M+H]⁺. ¹H NMR (400 MHz, DMSO-D6) δ 12.44 (s, 1H), 8.32 (s, 1H), 8.16 (d, J=8.8 Hz, 1H), 7.43 (dd, J=10.6, 5.9 Hz, 2H), 7.33-7.23 (m, 2H), 7.10 (d, J=2.3 Hz, 1H), 6.42 (s, 1H), 4.87-4.79 (m, 1H), 4.31 (s, 2H), 3.93 (s, 3H), 3.73 (m, 6H), 3.29 (s, 2H), 2.39 (m, 2H), 2.00 (m, 2H), 1.84-1.63 (m, 2H).

### Synthesis of Example 138: 6-(4-(5-((7-cyclobutoxy-4-oxo-3,4-dihydrophthalazin-1-yl)methyl)-2-fluorobenzoyl)piperazin-1-yl)-5-methylnicotinonitrile

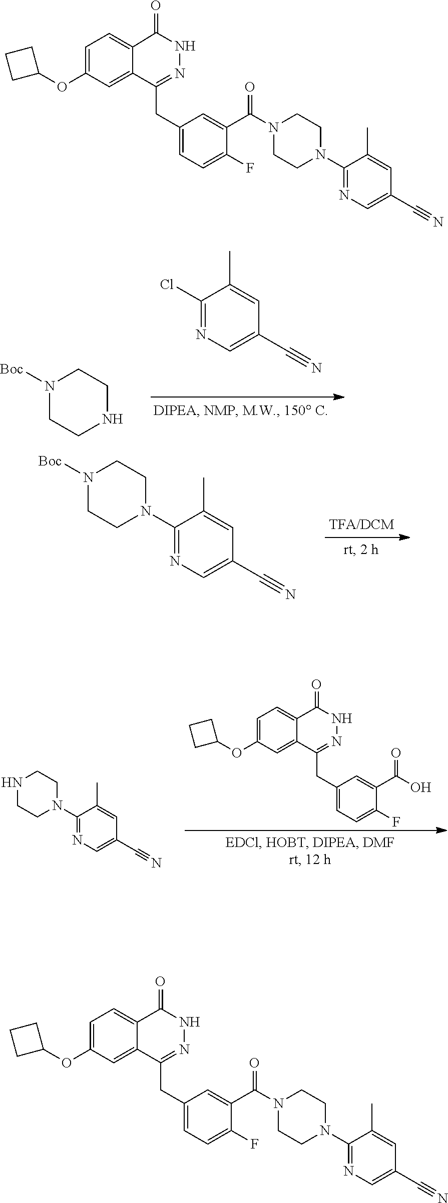

### Step 1

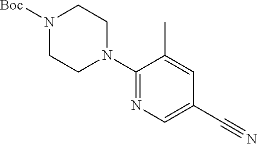 [SMILES: Cc1cc(C#N)cnc1N1CCN(C(=O)OC(C)(C)C)CC1]

### tert-butyl 4-(5-cyano-3-methylpyridin-2-yl)piperazine-1-carboxylate

6-chloro-5-methyl-pyridine-3-carbonitrile (100 mg, 655.39 μmol), tert-butyl piperazine-1-carboxylate (161.42 mg, 655.39 μmol, CH₃COOH), DIPEA (423.52 mg, 3.28 mmol, 570.79 μL) and NMP (0.5 mL) were added to a 10 mL sealed tube. The reaction mixture was stirred at 150° C. for 40 min under microwave. The mixture was diluted in EA (10 mL), washed with water (10 mL*2), brine, dried and concentrated to the crude. The residue was purified by silica gel chromatography (PE:EA=10:1 to 1:1) to give tert-butyl 4-(5-cyano-3-methyl-2-pyridyl)piperazine-1-carboxylate (150 mg, 75.6% yield) as a yellow solid. LCMS EST m/z 247.2 [M-56]⁺.

### Step 2

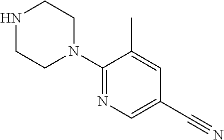

### 5-methyl-6-(piperazin-1-yl)nicotinonitrile

tert-butyl 4-(5-cyano-3-methyl-2-pyridyl)piperazine-1-carboxylate (150 mg, 496.08 μmol), TFA (1.48 g, 12.98 mmol, 1 mL) and DCM (2 mL) were added to a 100 mL bottled flask. The resultant mixture was stirred at 25° C. for 2 hr. The mixture was concentrated to give 5-methyl-6-piperazin-1-yl-pyridine-3-carbonitrile (150 mg, crude, TFA) as a yellow oil. LCMS ESI m/z: 203.2 [M+H]⁺.

### Step 3

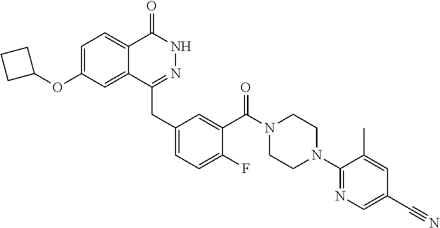 [SMILES: Cc1cc(C#N)cnc1N1CCN(C(=O)c2cc(Cc3n[nH]c(=O)c4ccc(OC5CCC5)cc34)ccc2F)CC1]

### 6-(4-(5-((7-cyclobutoxy-4-oxo-3,4-dihydrophthalazin-1-yl)methyl)-2-fluorobenzoyl)piperazin-1-yl)-5-methylnicotinonitrile

Following the general procedure above in Example 55, but starting with 5-methyl-6-(piperazin-1-yl)nicotinonitrile gave the title compound as a white solid. LCMS ESI m/z: 553.2 [M+H]⁺. ¹H NMR (40) MHz, DMSO-D6) δ 12.44 (s, 1H), 8.51 (d, J=2.1 Hz, 1H), 8.15 (d, J=8.8 Hz, 1H), 7.92 (d, J=1.5 Hz, 1H), 7.46-7.38 (m, 2H), 7.32-7.22 (m, 2H), 7.10 (d, J=2.3 Hz, 1H), 4.87-4.79 (m, 1H), 4.31 (s, 2H), 3.77 (s, 2H), 3.38-3.32 (m, 4H), 3.23 (s, 2H), 2.43-2.35 (m, 2H), 2.26 (s, 3H), 2.05-1.95 (m, 2H), 1.82-1.63 (m, 2H).

### Synthesis of Example 139: 6-(7-(5-((7-cyclobutoxy-4-oxo-3,4-dihydrophthalazin-1-yl)methyl)-2-fluorobenzoyl)-5,6,7,8-tetrahydro-[1,2,4]triazolo[4,3-a]pyrazin-3-yl)nicotinonitrile

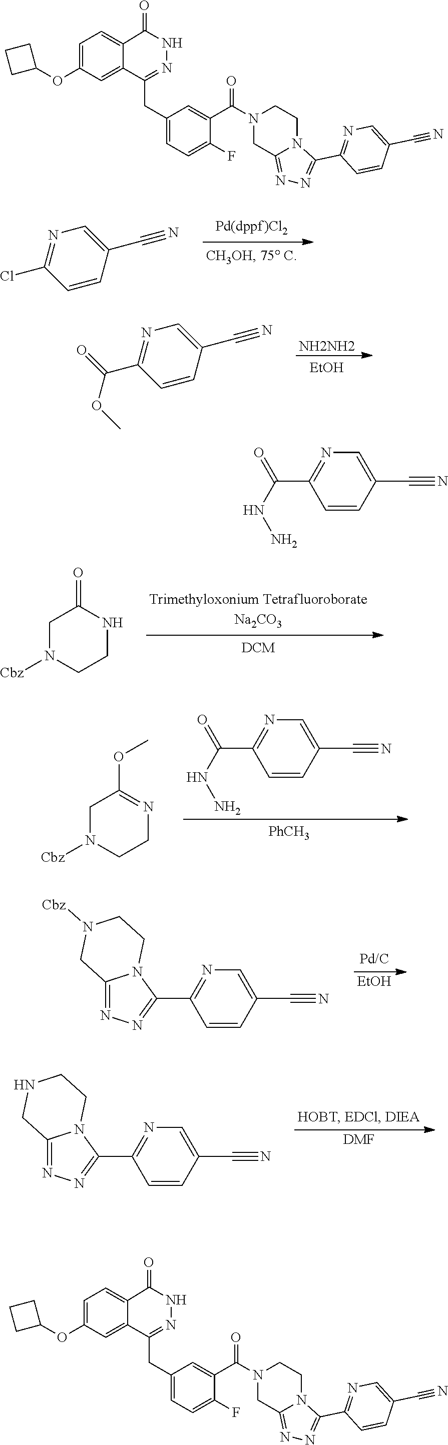

### Step 1

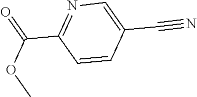

### methyl 5-cyanopicolinate

A mixture of 6-chloropyridine-3-carbonitrile (500) mg, 3.61 mmol), ET3N (730.32 mg, 7.22 mmol, 1.01 mL), Pd(dppf)Cl₂ (264.05 mug, 360.87 μmol) and Methanol (10 mL) was stirred at 75° C. for 12 hr under CO (g) (13 psi). The mixture was concentrated to a crude residue, which was purified by silica gel chromatography (PE:EA=10:1 to 1:1) to give methyl 5-cyanopyridine-2-carboxylate (120 mg, 20.5% yield) as a yellow solid. ¹H NMR (400 MHz, DMSO-D6) δ 9.20 (m, 1H), 8.53 (dd, J=8.2, 2.1 Hz, 1H), 8.20 (dd, J=8.2, 0.6 Hz, 1H), 3.92 (s, 3H).

### Step 2, 4, 5 and 6

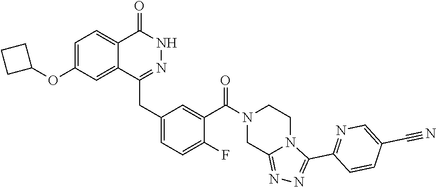 [SMILES: N#Cc1ccc(-c2nnc3n2CCN(C(=O)c2cc(Cc4n[nH]c(=O)c5ccc(OC6CCC6)cc45)ccc2F)C3)nc1]

### 6-(7-(5-((7-cyclobutoxy-4-oxo-3,4-dihydrophthalazin-1-yl)methyl)-2-fluorobenzoyl)-5,6,7,8-tetrahydro-[1,2,4]triazolo[4,3-a]pyrazin-3-yl)nicotinonitrile

Following the general four-step procedure in Example 1, but starting with methyl 5-cyanopicolinate gave the title compound as a white solid. LCMS ESI m/z: 577.2 [M+H]⁺. ¹H NMR (400 MHz, DMSO-D6) δ 12.43 (s, 1H), 9.15 (s, 1H), 8.47 (dd, J=8.4, 2.1 Hz, 1H), 8.34 (d, J=8.3 Hz, 1H), 8.15 (d, J=8.7 Hz, 1H), 7.48 (t, J=6.1 Hz, 2H), 7.31 (dd, J=10.3, 7.1 Hz, 2H), 7.08 (s, 1H), 5.05 (s, 1H), 4.87-4.77 (m, 1H), 4.72 (s, 1H), 4.56 (s, 1H), 4.40 (s, 1H), 4.32 (s, 2H), 4.13 (s, 1H), 3.68 (s, 1H), 2.37 (s, 2H), 2.07-1.91 (m, 2H), 1.83-1.60 (m, 2H).

### Synthesis of Example 140: 6-cyclobutoxy-4-(4-fluoro-3-(4-(5-hydroxypyridin-2-yl)piperazine-1-carbonyl)benzyl)phthalazin-1(2H)-one

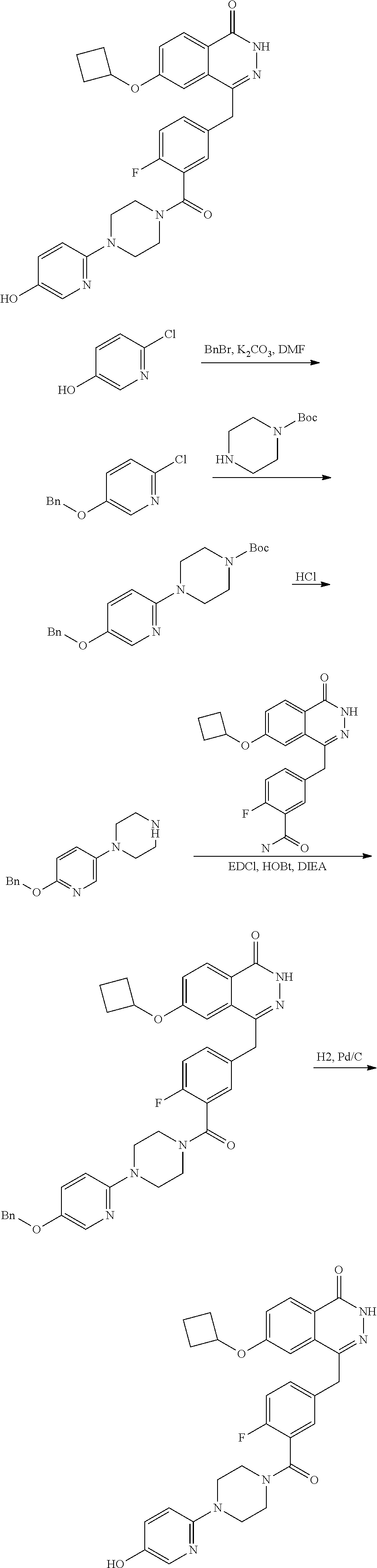

### Step 1

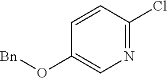 [SMILES: Clc1ccc(OCc2ccccc2)cn1]

### 5-(benzyloxy)-2-chloropyridine

A mixture of 6-chloropyridin-3-ol (1.00 g, 7.75 mmol). (bromomethyl)benzene (1.58 g, 9.30 mmol) and K₂CO₃ (1.60 g, 11.63 mmol) in DMF (20 mL) was stirred at 130° C. for 12 hours. The reaction was cooled to rt, diluted with H₂O (50 mL) and extracted with EtOAc (3×30 mL). The combined organic layer was washed with brine, dried over Na₂SO₄, filtered and concentrated in vacuo to give 5-(benzyloxy)-2-chloropyridine (1.25 g, 74% yield) as a yellow solid. LCMS ESI m/z: 220 [M+H]⁺.

### Step 2

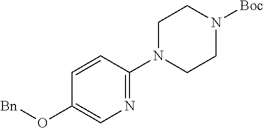 [SMILES: CC(C)(C)OC(=O)N1CCN(c2ccc(OCc3ccccc3)cn2)CC1]

### tert-butyl 4-(5-(benzyloxy)pyridin-2-yl)piperazine-1-carboxylate

A mixture of 5-(benzyloxy)-2-chloropyridine (1.25 g, 5.68 mmol), tert-butyl piperazine-1-carboxylate (1.06 g, 5.68 mmol), Pd₂(dba)₃ (530 mg, 0.57 mmol), BINAP (706 mg, 1.13 mmol) and t-BuONa (1.64 g, 17.04 mmol) in toluene (50 mL) was stirred at 110° C. under Ar (g) for 2 hours. The reaction was cooled to r, diluted with H₂O (50 mL) and extracted with EtOAc (50 mL×3). The combined organic layer was washed with brine, dried over anhydrous Na₂SO₄, filtered and concentrated in vacuo. The residue was purified by silica gel chromatography (EtOAc/pet ether=10% to 30%) to give tert-butyl 4-(5-cyanopyridin-2-yl)piperazine-1-carboxylate (1.05 g, 50% yield) as a white solid. LCMS ESI m/z: 370 [M+H]⁺.

### Step 3

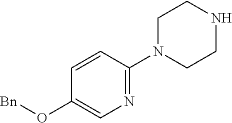 [SMILES: c1ccc(COc2ccc(N3CCNCC3)nc2)cc1]

### 1-(5-(benzyloxy)pyridin-2-yl)piperazine

A solution of tert-butyl 4-(5-(benzyloxy)pyridin-2-yl)piperazine-1-carboxylate (370 mg, 1 mmol) in HCl/dioxane (4M, 5 mL) was stirred at rt for 1 hour. The solvent was removed in vacuo to give 1-(5-(benzyloxy)pyridin-2-yl)piperazine (240 mg, 94% yield) as a white solid. This was used without further purification. (ESI 432 [M+H]⁺). LCMS ESI m/z: 270 [M+H]⁺.

### Step 4

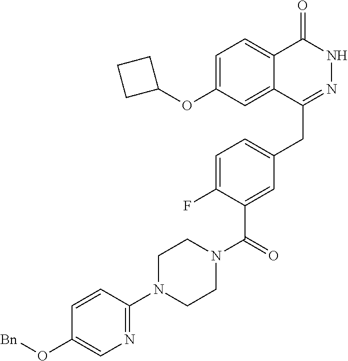 [SMILES: O=C(c1cc(Cc2n[nH]c(=O)c3ccc(OC4CCC4)cc23)ccc1F)N1CCN(c2ccc(OCc3ccccc3)cn2)CC1]

### 4-(3-(4-(5-(benzyloxy)pyridin-2-yl)piperazine-1-carbonyl)-4-fluorobenzyl)-6-cyclobutoxyphthalazin-1(2H)-one

To a solution of 1-(5-(benzyloxy)pyridin-2-yl)piperazine (73 mg, 0.27 mmol) and 5-((7-cyclobutoxy-4-oxo-3,4-dihydrophthalazin-1-yl)methyl)-2-fluorobenzoic acid (100 mg, 0.27 mmol) in DMF (2 mL) was added EDCI (52 mg, 0.27 mmol), HOBT (55 mg, 0.41 mmol) and DIPEA (105 mg, 0.81 mmol). The reaction mixture was stirred at rt for 2 hours. The residue was purified by prep-HPLC to afford 4-(3-(4-(5-(benzyloxy)pyridin-2-yl)piperazine-1-carbonyl)-4-fluorobenzyl)-6-cyclobutoxyphthalazin-1(2H)-one (90 mg, 54% yield) as a white solid. LCMS ESI m/z: 620 [M+H]⁺.

### Step 5

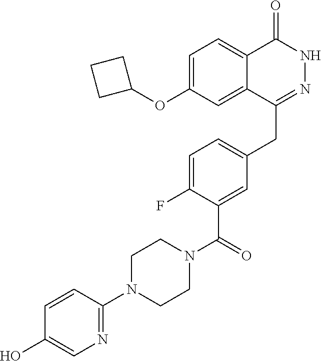 [SMILES: O=C(c1cc(Cc2n[nH]c(=O)c3ccc(OC4CCC4)cc23)ccc1F)N1CCN(c2ccc(O)cn2)CC1]

### 6-cyclobutoxy-4-(4-fluoro-3-(4-(5-hydroxypyridin-2-yl)piperazine-1-carbonyl)benzyl)phthalazin-1(2H)-one

A mixture of 4-(3-(4-(5-(benzyloxy)pyridin-2-yl)piperazine-1-carbonyl)-4-fluorobenzyl)-6-cyclobutoxyphthalazin-1(2H)-one (90 mg, 0.15 mmol) and Pd/C (18 mg) in MeOH (5 mL) was stirred under H₂ at room temperature for 2 hours. The mixture was filtered and concentrated in vacuo. The residue was purified by prep-HPLC to give 6-cyclobutoxy-4-(4-fluoro-3-(4-(5-hydroxypyridin-2-yl)piperazine-1-carbonyl)benzyl)phthalazin-1(2H)-one (53 mg, 67% yield) as a white solid. LCMS ESI m/z: 530 [M+H]⁺. ¹H-NMR (400 MHz, DMSO-d₆) δ 12.45 (s, 1H), 9.08 (s, 1H), 8.15 (d, J=8.8 Hz, 1H), 7.75 (d, J=3.2 Hz, 1H), 7.42 (d, J=3.2 Hz, 1H), 7.40-7.22 (m, 4H), 7.10-7.07 (m, 2H), 4.84-4.81 (m, 1H), 4.30 (s, 2H), 3.72 (s, 2H), 3.36-3.22 (m, 6H), 2.42-2.38 (m, 2H), 2.07-1.75 (m, 4H).

### Synthesis of Example 141: 5-(4-(5-((7-cyclobutoxy-4-oxo-3,4-dihydrophthalazin-1-yl)methyl)-2-fluorobenzoyl) piperazin-1-yl)nicotinonitrile

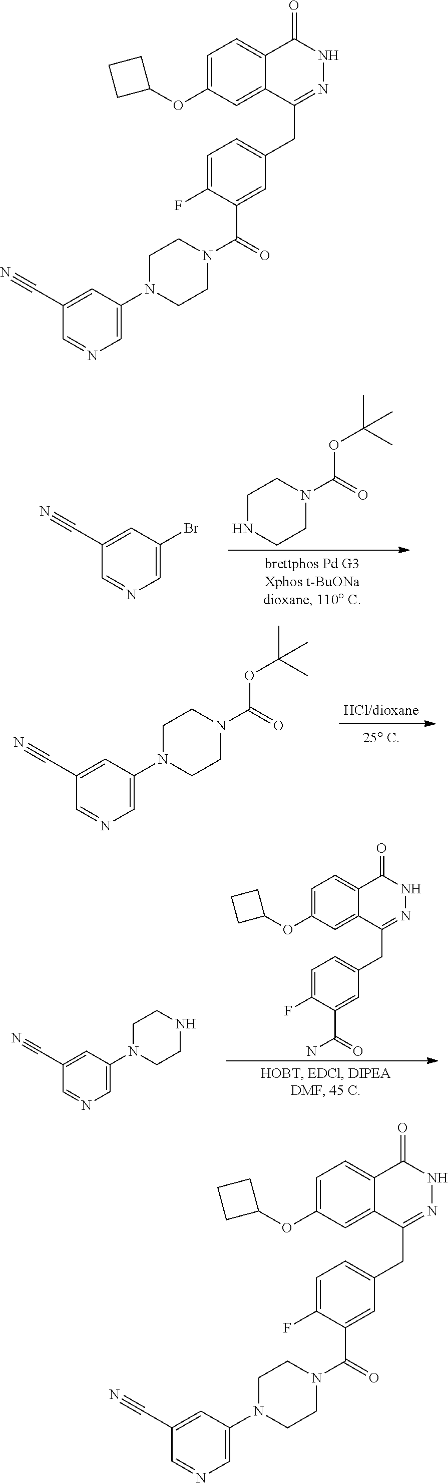

### Step 1

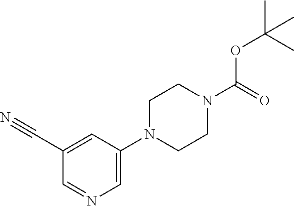 [SMILES: CC(C)(C)OC(=O)N1CCN(c2cncc(C#N)c2)CC1]

### tert-butyl 4-(5-cyanopyridin-3-yl)piperazine-1-carboxylate

A mixture of 5-bromonicotinonitrile (300 mg, 1.64 mmol), tert-butyl piperazine-1-carboxylate (458 mg, 2.46 mmol), brettphos Pd G3 (143 mg, 0.16 mmol), Xphos (313 mg, 0.65 mmol) and t-BuONa (315 mg, 3.28 mmol) in dioxane (3 mL) was stirred at 110° C. under Ar (g) for 16 hours. The mixture was cooled to rt, diluted with H₂O (10 mL) and extracted with EtOAc (20 mL×3). The combined organic layer was washed with brine, dried over anhydrous Na₂SO₄, filtered and concentrated in vacuo. The residue was purified by silica gel chromatography (EtOAc/pet ether=20% to 30%) to give tert-butyl 4-(5-cyanopyridin-3-yl)piperazine-1-carboxylate (138 mg, 29% yield) as a white solid. LCMS ESI m/z: 289 [M+H]⁺.

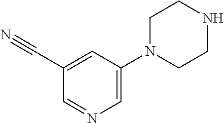 [SMILES: N#Cc1cncc(N2CCNCC2)c1]

### 5-(piperazin-1-yl)nicotinonitrile

A solution of tert-butyl 4-(5-cyanopyridin-3-yl)piperazine-1-carboxylate (138 mg, 0.48 mmol) in HCl/dioxane (4M, 2 mL) was stirred at 25° C. for 1 hour. The reaction mixture was concentrated in vacuo to give 5-(piperazin-1-yl)nicotinonitrile as a white solid (90 mg, 99% yield). LCMS ESI m/z: 189 [M+H]⁺. This was used without further purification.

### Step 3

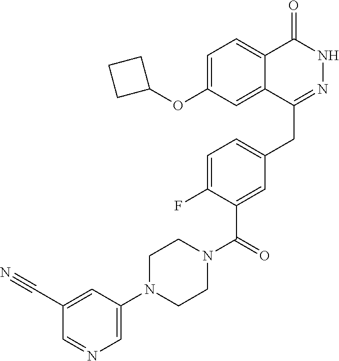 [SMILES: N#Cc1cncc(N2CCN(C(=O)c3cc(Cc4n[nH]c(=O)c5ccc(OC6CCC6)cc45)ccc3F)CC2)c1]

### 5-(4-(5-((7-cyclobutoxy-4-oxo-3,4-dihydrophthalazin-1-yl)methyl)-2-fluorobenzoyl) piperazin-1-yl)nicotinonitrile

To a solution of 5-(piperazin-1-yl)nicotinonitrile (79 mg, 0.26 mmol) and 5-((7-cyclobutoxy-4-oxo-3,4-dihydrophthalazin-1-yl)methyl)-2-fluorobenzoic acid (80 mg, 0.22 mmol) in DMF (2 mL) was added EDCI (51 mg, 0.33 mmol), HOBt (44 mg, 0.33 mmol) and DIPEA (140 mg, 0.65 mmol). The reaction mixture was stirred at 45° C. for 1 hour. The reaction was purified by prep-HPLC to give 5-(4-(5-((7-cyclobutoxy-4-oxo-3,4-dihydrophthalazin-1-yl)methyl)-2-fluorobenzoyl)piperazin-1-yl)nicotinonitrile (36 mg, 31% yield) as a white solid. LCMS ESI m/z: 539 [M+H]⁺. ¹H-NMR (400 MHz, DMSO-D6) δ 12.46 (s, 1H), 8.59 (d, J=2.9 Hz, 1H), 8.38 (d, J=1.6 Hz, 1H), 8.16 (d, J=8.8 Hz, 1H), 7.79 (q, J=2.8, 1.7 Hz, 1H), 7.47-7.38 (m, 2H), 7.34-7.23 (m, 2H), 7.11 (d, J=2.3 Hz, 1H), 4.84 (p, J=7.1 Hz, 1H), 4.31 (s, 2H), 3.76 (s, 2H), 3.42-3.38 (m, 2H), 3.32-3.29 (m, 2H), 3.25 (s, 2H), 2.45-2.30 (m, 2H), 2.07-1.92 (m, 2H), 1.83-1.55 (m, 2H).

### Synthesis of Example 142: 6-Cyclobutoxy-4-(3-(5-(cyclopropanecarbonyl)-2,5-diazabicyclo[2.2.2]octane-2-carbonyl)-4-fluorobenzyl)phthalazin-1(2H)-one

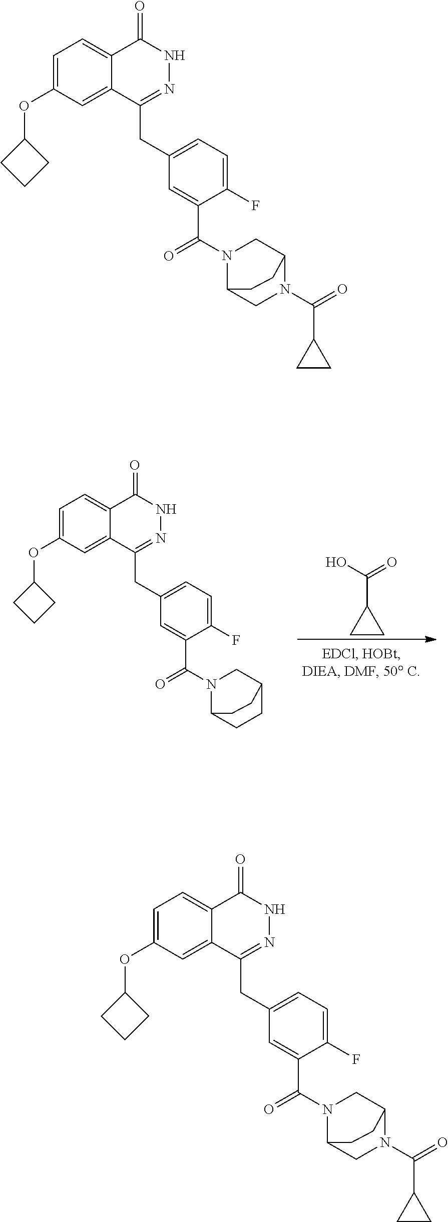 [SMILES: O=C(c1cc(Cc2n[nH]c(=O)c3ccc(OC4CCC4)cc23)ccc1F)N1CC2CCC1CN2C(=O)C1CC1 ; O=C(c1cc(Cc2n[nH]c(=O)c3ccc(OC4CCC4)cc23)ccc1F)N1CC2CCC1CC2 ; O=C(O)C1CC1 ; O=C(c1cc(Cc2n[nH]c(=O)c3ccc(OC4CCC4)cc23)ccc1F)N1CC2CCC1CN2C(=O)C1CC1]

### Step 1

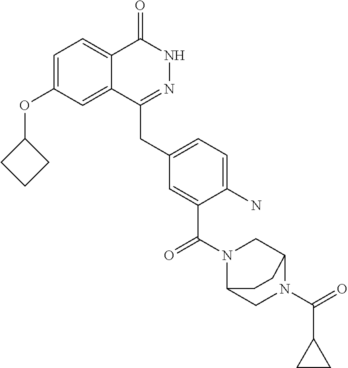 [SMILES: Nc1ccc(Cc2n[nH]c(=O)c3ccc(OC4CCC4)cc23)cc1C(=O)N1CC2CCC1CN2C(=O)C1CC1]

### 6-cyclobutoxy-4-(3-(5-(cyclopropanecarbonyl)-2,5-diazabicyclo[2.2.2]octane-2-carbonyl)-4-fluorobenzyl)phthalazin-1(2H)-one

To a solution of 4-(3-(2,5-diazabicyclo[2.2.2]octane-2-carbonyl)-4-fluorobenzyl)-6-cyclobutoxyphthalazin-1(2H)-one (100 mg, 0.22 mmol) and cyclopropanecarboxylic acid (22 mg, 0.26 mmol) in DMF (3 mL) was added EDCI (62 mg, 0.32 mmol). HOBt (44 mg, 0.32 mmol) and DIPEA (84 mg, 0.65 mmol). The reaction mixture was stirred at 50° C. for 1 hour. The reaction was purified by prep-HPLC to give 6-cyclobutoxy-4-(3-(5-(cyclopropanecarbonyl)-2,5-diazabicyclo[2.2.2]octane-2-carbonyl)-4-fluorobenzyl) phthalazin-1(2H)-one (52 mg, 0.10 mmol, 45% yield) as a white solid. LCMS EST m/z: 531.2 [M+H]⁺. ¹H-NMR (400 MHz, DMSO-d6) δ 12.46 (d, J=3.7 Hz, 1H), 8.14 (dd, J=8.8, 1.7 Hz, 1H), 7.52-7.41 (m, 1H), 7.37 (dd, J=6.7, 5.0 Hz, 1H), 7.32-7.19 (m, 2H), 7.13-7.01 (m, 1H), 4.89-4.77 (m, 1H), 4.73-4.62 (m, 1H), 4.35-4.25 (m, 2H), 3.73-3.50 (m, 4H), 3.40 (d, J=11.8 Hz, 2H), 2.44-2.32 (m, 2H), 2.04-1.91 (m, 3H), 1.82-1.73 (m, 3H), 1.69-1.56 (m, 2H), 0.78-0.65 (m, 4H).

### Synthesis of Example 143: 6-(3-(5-((7-cyclobutoxy-4-oxo-3,4-dihydrophthalazin-1-31)methyl)-2-fluorobenzoyl)-3,8-diazabicyclo[3.2.1]octan-8-yl)nicotinonitrile

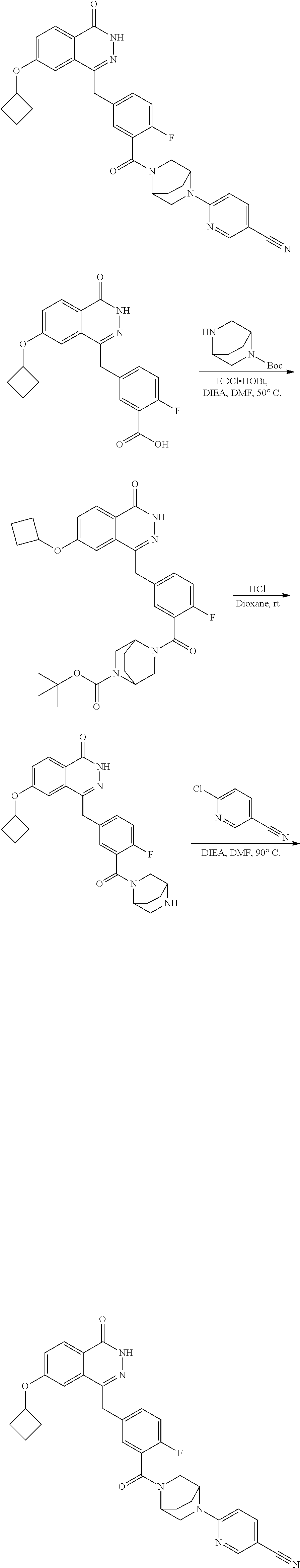

### Step 1

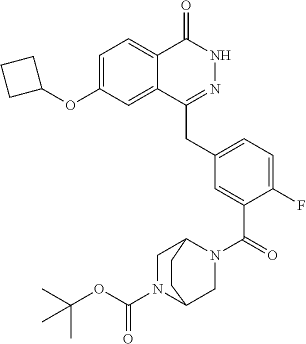 [SMILES: CC(C)(C)OC(=O)N1CC2CCC1CN2C(=O)c1cc(Cc2n[nH]c(=O)c3ccc(OC4CCC4)cc23)ccc1F]

### tert-butyl 5-(5-((7-cyclobutoxy-4-oxo-3,4-dihydrophthalazin-1-yl)methyl)-2-fluorobenzoyl)-2,5-diazabicyclo[2.2.2]octane-2-carboxylate

To a solution of 5-((7-cyclobutoxy-4-oxo-3,4-dihydrophthalazin-1-yl)methyl)-2-fluorobenzoic acid (300 mg, 0.81 mmol) and tert-butyl 2,5-diazabicyclo[2.2.2]octane-2-carboxylate (173 mg, 0.81 mmol) in DMF (3 mL) was added EDCI (165 mg, 1.22 mmol). HOBt (234 mg, 1.22 mmol) and DIPEA (316 mg, 2.44 mmol). The reaction mixture was stirred at rt for 16 hours. The mixture was diluted with H₂O (10 mL) and extracted with EtOAc (10 mL×3). The combined organic layer was washed with brine, dried over anhydrous Na₂SO₄, filtered and concentrated in vacuo. The crude was purified by silica gel chromatography (PE:EA=9:1 to 1:1) to give tert-butyl methyl(3-oxo-3-(4-(5-(trifluoromethyl)pyrimidin-2-yl)piperazin-1-yl)propyl)carbamate (408 mg, 0.73 mmol, 89% yield) as a white solid. LCMS EST m/z: 563.2 [M+H]⁺.

### Step 2

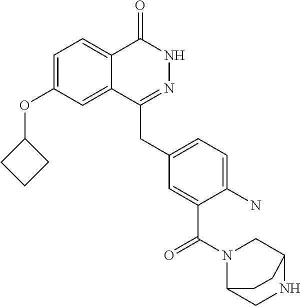 [SMILES: Nc1ccc(Cc2n[nH]c(=O)c3ccc(OC4CCC4)cc23)cc1C(=O)N1CC2CCC1CN2]

### 4-(3-(2,5-diazabicyclo[2.2.2]octane-2-carbonyl)-4-fluorobenzyl)-6-cyclobutoxyphthalazin-1(2H)-one

To a solution of tert-butyl 5-(5-((7-cyclobutoxy-4-oxo-3,4-dihydrophthalazin-1-yl)methyl)-2-fluorobenzoyl)-2,5-diazabicyclo[2.2.2]octane-2-carboxylate (408 mg, 0.73 mmol) in dioxane (5 mL) was added HCl/dioxane (4M, 5 mL) and stirred at 25° C. for 1 hour. The mixture was concentrated in vacuo to afford 4-(3-(2,5-diazabicyclo[2.2.2]octane-2-carbonyl)-4-fluorobenzyl)-6-cyclobutoxyphthalazin-1(2H)-one (361 mg, 0.72 mmol, 99% yield) as a brown oil. This was used without further purification. LCMS ESI m/z: 463.2 [M+H]⁺.

### Step 3

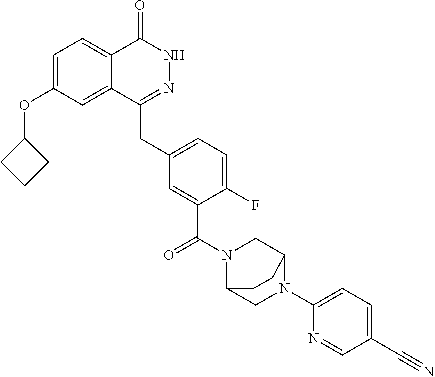 [SMILES: N#Cc1ccc(N2CC3CCC2CN3C(=O)c2cc(Cc3n[nH]c(=O)c4ccc(OC5CCC5)cc34)ccc2F)nc1]

### 6-(5-(5-((7-cyclobutoxy-4-oxo-3,4-dihydrophthalazin-1-yl)methyl)-2-fluorobenzoyl)-2,5-diazabicyclo[2.2.2]octan-2-yl)nicotinonitrile

To a solution of 4-(3-(2,5-diazabicyclo[2.2.2]octane-2-carbonyl)-4-fluorobenzyl)-6-cyclobutoxyphthalazin-1(2H)-one (80 mg, 0.17 mmol) and 6-chloronicotinonitrile (29 mg, 0.21 mmol) in DMF (1 mL) was added DIPEA (67 mg, 0.51 mmol). The reaction mixture was stirred at 90° C. for 16 hours. The reaction was purified by prep-HPLC to give 6-(5-(5-((7-cyclobutoxy-4-oxo-3,4-dihydrophthalazin-1-yl)methyl)-2-fluorobenzoyl)-2,5-diazabicyclo[2.2.2]octan-2-yl)nicotinonitrile (31 mg, 0.05 mmol, 32% yield) as a white solid. LCMS ESI m/z: 565.0 [M+H]⁺. ¹H-NMR (400 MHz, DMSO-d6) δ 12.46 (d, J=7.3 Hz, 1H), 8.48 (t, J=4.9 Hz, 1H), 8.16 (dd, J=12.7, 8.8 Hz, 1H), 7.87 (d, J=7.2 Hz, 1H), 7.45 (dd, J=5.8, 2.5 Hz, 1H), 7.36 (dd, J=7.6, 5.5 Hz, 1H), 7.34-7.29 (m, 1H), 7.29-7.18 (m, 1H), 7.08 (d, J=1.9 Hz, 1H), 6.62 (s, 1H), 4.83 (dd, J=14.2, 7.0 Hz, 2H), 4.32 (t, J=14.7 Hz, 2H), 3.65 (t, J=17.5 Hz, 3H), 3.48 (d, J=27.7 Hz, 2H), 2.43-2.33 (m, 2H), 1.99 (d, J=8.1 Hz, 2H), 1.89 (s, 3H), 1.80-1.59 (m, 3H).

### Synthesis of Example 144: 6-cyclobutoxy-4-(4-fluoro-3-(5-(5-(trifluoromethyl)pyrimidin-2-yl)-2,5-diazabicyclo[2.2.2]octane-2-carbonyl)benzyl)phthalazin-1(2H)-one

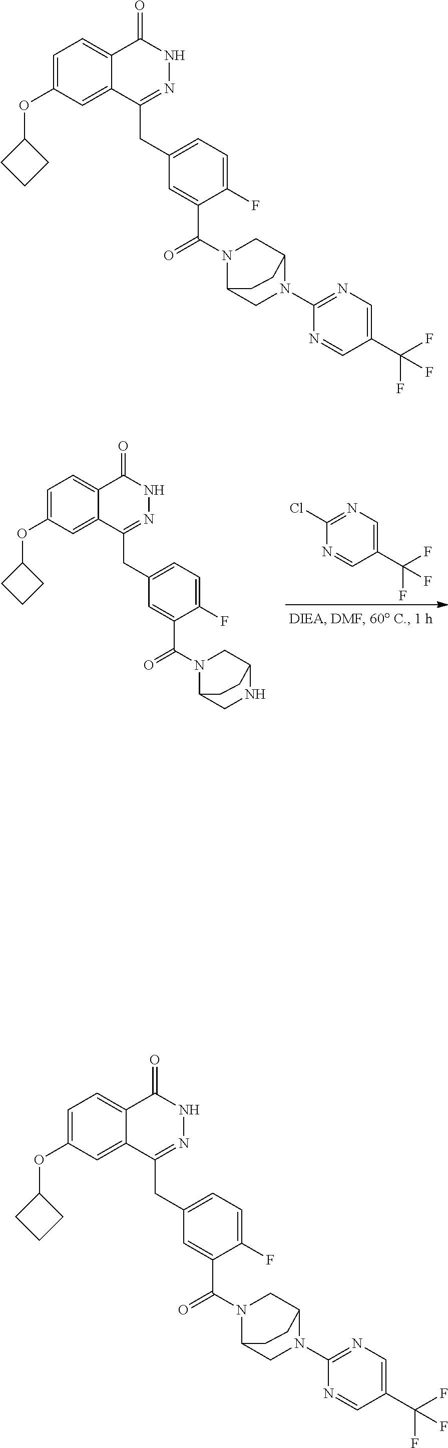 [SMILES: O=C(c1cc(Cc2n[nH]c(=O)c3ccc(OC4CCC4)cc23)ccc1F)N1CC2CCC1CN2c1ncc(C(F)(F)F)cn1 ; O=C(c1cc(Cc2n[nH]c(=O)c3ccc(OC4CCC4)cc23)ccc1F)N1CC2CCC1CN2 ; FC(F)(F)c1cnc(Cl)nc1 ; O=C(c1cc(Cc2n[nH]c(=O)c3ccc(OC4CCC4)cc23)ccc1F)N1CC2CCC1CN2c1ncc(C(F)(F)F)cn1]

### Step 1

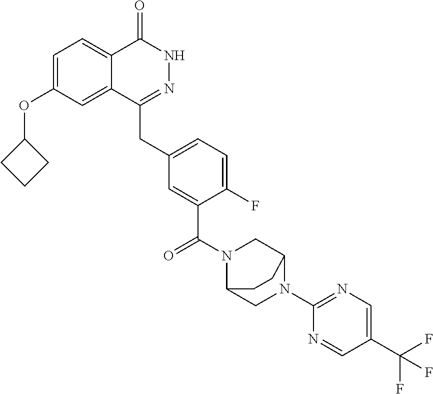 [SMILES: O=C(c1cc(Cc2n[nH]c(=O)c3ccc(OC4CCC4)cc23)ccc1F)N1CC2CCC1CN2c1ncc(C(F)(F)F)cn1]

### 6-cyclobutoxy-4-(4-fluoro-3-(5-(5-(trifluoromethyl)pyrimidin-2-yl)-2,5-diazabicyclo[2.2.2]octane-2-carbonyl)benzyl)phthalazin-1(2H)-one

To a solution of 4-(3-(2,5-diazabicyclo[2.2.2]octane-2-carbonyl)-4-fluorobenzyl)-6-cyclobutoxyphthalazin-1(2H)-one (120 mg, 0.15 mmol), 2-chloro-5-trifluoromethyl) pyrimidine (37 mg, 0.20 mmol) in DMF (1 mL) was added DIPEA (100 mg, 0.78 mmol). The reaction mixture was stirred at 90° C. for 1 hour. The mixture was purified by prep-HPLC to give 6-cyclobutoxy-4-(4-fluoro-3-(5-(5-(trifluoromethyl)pyrimidin-2-yl)-2,5-diazabicyclo[2.2.2]octane-2-carbonyl) benzyl) phthalazin-1(2H)-one (62 mg, 40% yield) as a white solid. LCMS ESI m/z: 609.2 [M+H]⁺. ¹H-NMR (400 MHz, CDCl3) δ 10.73 (s, 1H), 8.54 (d, J=2.9 Hz, 1H), 8.45 (dd, J=23.1, 2.4 Hz, 1H), 8.35 (t, J=8.0 Hz, 1H), 7.37-7.27 (m, 2H), 7.19 (dd, J=8.8, 2.3 Hz, 1H), 7.07 (t, J=8.7 Hz, 1H), 6.92-6.87 (m, 1H), 5.21 (d, 1H), 4.71-4.61 (m, 1H), 4.22 (d, J=8.2 Hz, 2H), 3.97-3.71 (m, 4H), 3.58 (d, J=11.0 Hz, 1H), 2.47-2.35 (m, 2H), 2.18-1.85 (m, 6H), 1.83-1.73 (m, 2H).

### Synthesis of Example 145: 6-cyclobutoxy-4-(4-fluoro-3-(3-(trifluoromethyl)-5,6,7,8-tetrahydro-[1,2,4]triazolo[4,3-a]pyrazine-7-carbonyl)benzyl)phthalazin-1(2H)-one

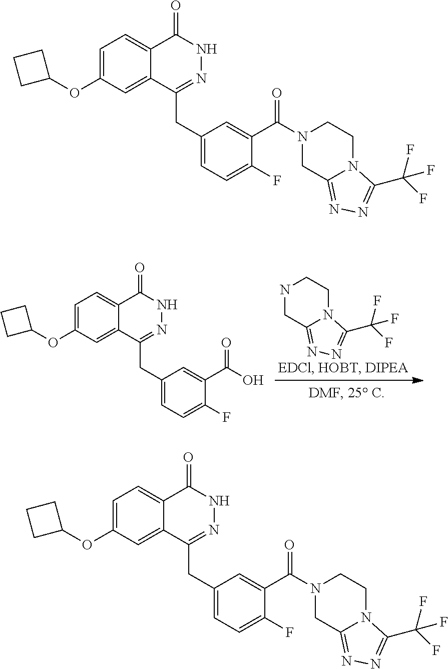

### Step 1

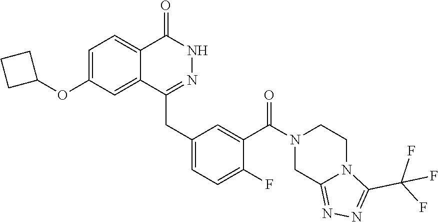 [SMILES: O=C(c1cc(Cc2n[nH]c(=O)c3ccc(OC4CCC4)cc23)ccc1F)N1CCn2c(nnc2C(F)(F)F)C1]

### 6-cyclobutoxy-4-(4-fluoro-3-(3-(trifluoromethyl)-5,6,7,8-tetrahydro-[1,2,4]triazolo[4,3-a]pyrazine-7-carbonyl)benzyl)phthalazin-1(2H)-one

Following the procedure above in Example 1, but starting with 5-((7-cyclobutoxy-4-oxo-3,4-dihydrophthalazin-1-yl)methyl)-2-fluorobenzoic acid gave the title compound as a white solid. LCMS ESI m/z: 542.7 [M+H]⁺. ¹H NMR (400 MHz, DMSO-d6) δ 12.43 (s, 1H), 8.15 (d, J=8.8 Hz, 1H), 7.54-7.47 (m, 1H), 7.44 (d, J=5.4 Hz, 1H), 7.36-7.24 (m, 2H), 7.06 (d, J=13.4 Hz, 1H), 5.02 (s, 1H), 4.86-4.77 (m, 1H), 4.69 (s, 1H), 4.31 (s, 2H), 4.18 (m, 2H), 3.99 (s, 1H), 3.68 (s, 1H), 2.43-2.31 (m, 2H), 2.05-1.93 (m, 2H), 1.78 (m, 1H), 1.64 (m, 1H).

### Synthesis of Example 146: 6-cyclobutoxy-4-(3-(3-cyclopropyl-5,6,7,8-tetrahydro-[1,2,4]triazolo[4,3-a]pyrazine-7-carbonyl)-4-fluorobenzyl)phthalazin-1(2H)-one

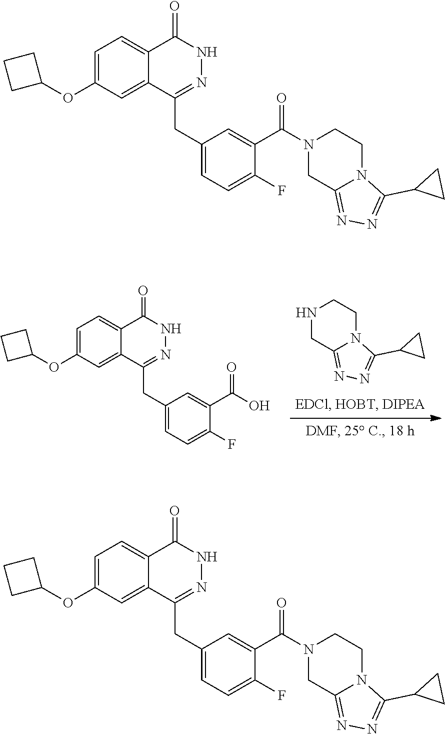 [SMILES: O=C(c1cc(Cc2n[nH]c(=O)c3ccc(OC4CCC4)cc23)ccc1F)N1CCn2c(nnc2C2CC2)C1 ; O=C(O)c1cc(Cc2n[nH]c(=O)c3ccc(OC4CCC4)cc23)ccc1F ; C1Cn2c(nnc2C2CC2)CN1 ; O=C(c1cc(Cc2n[nH]c(=O)c3ccc(OC4CCC4)cc23)ccc1F)N1CCn2c(nnc2C2CC2)C1]

### Step 1

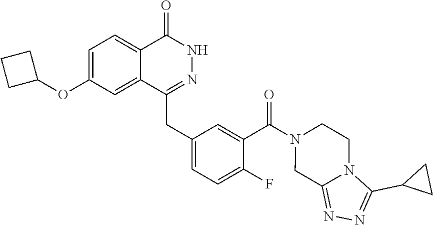 [SMILES: O=C(c1cc(Cc2n[nH]c(=O)c3ccc(OC4CCC4)cc23)ccc1F)N1CCn2c(nnc2C2CC2)C1]

### 6-cyclobutoxy-4-(3-(3-cyclopropyl-5,6,7,8-tetrahydro-[1,2,4]triazolo[4,3-a]pyrazine-7-carbonyl)-4-fluorobenzyl)phthalazin-1(2H)-one

Following the procedure above in Example 8, but starting with 3-cyclopropyl-5,6,7,8-tetrahydro-[1,2,4]triazolo[4,3-a]pyrazine gave the title compound as a white solid. LCMS ESI m/z: 514.8 [M+H]⁺. ¹H NMR (400 MHz, DMSO-d6) δ 12.43 (s, 1H), 8.15 (dd, J=8.6, 4.9 Hz, 1H), 7.46 (dd, J=10.7, 5.8 Hz, 2H), 7.31 (t, J=8.7 Hz, 2H), 7.07 (d, J=9.1 Hz, 1H), 4.83 (dd, J=14.3, 7.1 Hz, 2H), 4.53 (s, 1H), 4.31 (s, 2H), 4.09 (s, 1H), 3.82 (s, 1H), 3.62 (s, 1H), 2.36 (s, 2H), 2.04-1.72 (m, 4H), 1.65 (m, 1H), 1.25 (s, 1H), 1.02-0.93 (m, 2H), 0.87 (d, J=2.6 Hz, 2H).

### Synthesis of Example 147: 6-cyclobutoxy-4-(4-fluoro-3-(5-(5-(trifluoromethyl)pyrimidin-2-yl)octahydropyrrolo[3,4-c]pyrrole-2-carbonyl)benzyl)phthalazin-1(2H)-one

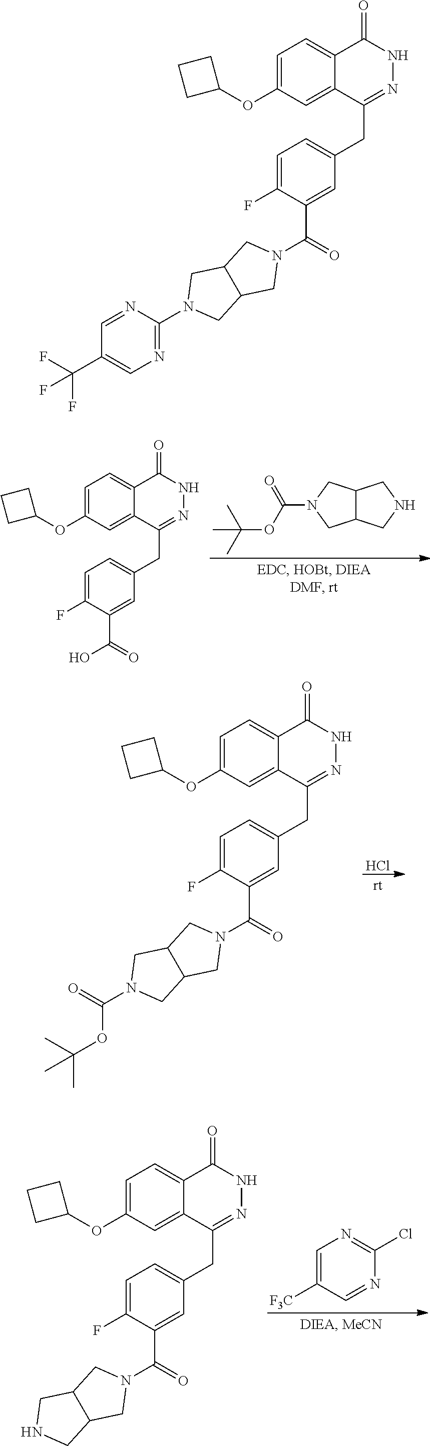

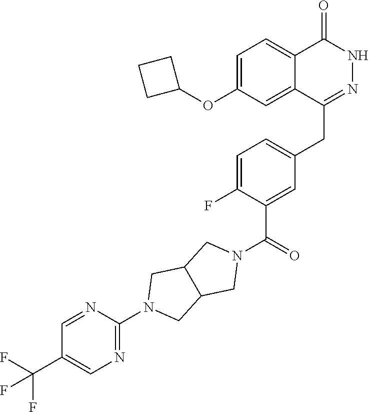 [SMILES: O=C(c1cc(Cc2n[nH]c(=O)c3ccc(OC4CCC4)cc23)ccc1F)N1CC2CN(c3ncc(C(F)(F)F)cn3)CC2C1]

### Step 1

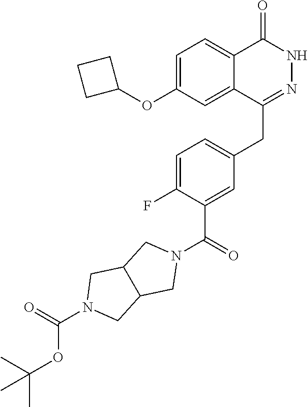 [SMILES: CC(C)(C)OC(=O)N1CC2CN(C(=O)c3cc(Cc4n[nH]c(=O)c5ccc(OC6CCC6)cc45)ccc3F)CC2C1]

### tert-butyl 5-(5-((7-cyclobutoxy-4-oxo-3,4-dihydrophthalazin-1-yl)methyl)-2-fluorobenzoyl)hexahydropyrrolo[3,4-c]pyrrole-2(1H)-carboxylate

To a solution of 5-((7-cyclobutoxy-4-oxo-3,4-dihydrophthalazin-1-yl)methyl)-2-fluorobenzoic acid (300 mg, 0.81 mmol) and tert-butyl hexahydropyrrolo[3,4-c]pyrrole-2(1H)-carboxylate (207 mg, 0.98 mmol) in DMF (2 mL) was added EDCI (234 mg, 1.22 mmol), HOBt (165 mg, 1.22 mmol) and DIPEA (526 mg, 4.07 mmol). The reaction mixture was stirred at rt for 16 hours. The mixture was diluted with H₂O (50 mL) and extracted with EtOAc (50 mL×3). The combined organic layer was washed with brine, dried over anhydrous Na₂SO₄, filtered and concentrated in vacuo. The residue was purified by silica gel chromatography (EtOAc/pet ether=40% to 50%) to give tert-butyl 5-(5-((7-cyclobutoxy-4-oxo-3,4-dihydrophthalazin-1-yl)methyl)-2-fluorobenzoyl)hexa hydropyrrolo[3,4-c]pyrrole-2(1H)-carboxylate (436 mg 95% yield) as a white solid, LCMS ESI 563 [M+H]⁺.

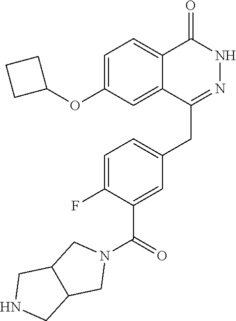 [SMILES: O=C(c1cc(Cc2n[nH]c(=O)c3ccc(OC4CCC4)cc23)ccc1F)N1CC2CNCC2C1]

### 6-cyclobutoxy-4-(4-fluoro-3-(octahydropyrrolo[3,4-c]pyrrole-2-carbonyl)benzyl)phthalazin-1(2H)-one

To a solution of tert-butyl 5-(5-((7-cyclobutoxy-4-oxo-3,4-dihydrophthalazin-1-yl)methyl)-2-fluorobenzoyl)hexahydropyrrolo[3,4-c]pyrrole-2(1H)-carboxylate (436 mg, 0.75 mmol)) in dioxane (2 mL) was added HCl/dioxane (4.0M, 4 mL, 16.00 mmol). The mixture was stirred at rt for 1 hour. The solvent was removed in vacuo to give 6-cyclobutoxy-4-(4-fluoro-3-(octahydropyrrolo[3,4-c]pyrrole-2-carbonyl)benzyl)phthalazin-1(2H)-one (322 mg 90% yield) as a white solid. This was used without further purification. LCMS ESI 463 [M+H]⁺.

### Step 3

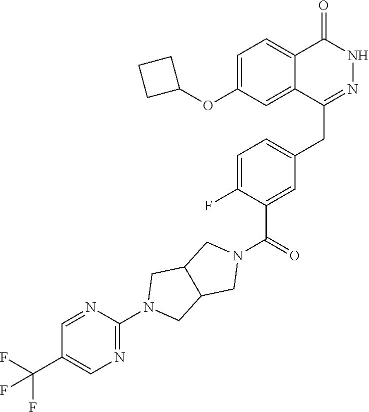 [SMILES: O=C(c1cc(Cc2n[nH]c(=O)c3ccc(OC4CCC4)cc23)ccc1F)N1CC2CN(c3ncc(C(F)(F)F)cn3)CC2C1]

### 6-cyclobutoxy-4-(4-fluoro-3-(5-(5-(trifluoromethyl)pyrimidin-2-yl)octahydropyrrolo[3,4-c]pyrrole-2-carbonyl)benzyl)phthalazin-1(2H)-one

A mixture of 6-cyclobutoxy-4-(4-fluoro-3-(octahydropyrrolo[3,4-c]pyrrole-2-carbonyl)benzyl)phthalazin-1(2H)-one (200 mg, 0.40 mmol), 2-chloro-5-(trifluoromethyl)pyrimidine (81 mg, 0.44 mmol) and DIPEA (207 mg, 1.60 mmol) in MeCN (3 mL) was stirred at 50° C. for 1 hour. The reaction was concentrated in vacuo. The residue was purified by prep-HPLC to give the desired product 6-cyclobutoxy-4-(4-fluoro-3-(5-(5-(trifluoromethyl)pyrimidin-2-yl)octahydropyrrolo[3,4-c]pyrrole-2-carbonyl)benzyl)phthalazin-1(2H)-one (85 mg, 35% yield) as a white solid. LCMS ESI 609.2 [M+H]⁺. ¹H NMR (400 MHz, DMSO-D6) δ 12.45 (s, 1H), 8.70 (d, J=2.1 Hz, 2H), 8.14 (d, J=8.8 Hz, 1H), 7.46-7.34 (m, 2H), 7.33-7.17 (m, 2H), 7.08 (d, J=2.3 Hz, 1H), 4.82 (p, J=7.1 Hz, 1H), 4.34-4.24 (m, 2H). 3.88-3.65 (m, 3H), 3.54-3.35 (m, 4H), 3.19-2.94 (m, 3H), 2.37 (m, 2H), 2.10-1.89 (m, 2H), 1.81-1.57 (m, 2H).

### Synthesis of Example 148: 6-(5-((7-Cyclobutoxy-4-oxo-3,4-dihydrophthalazin-1-yl)methyl)-2-fluorobenzoyl)-2,6-diazaspiro[3.3]heptan-2-yl)nicotinonitrile

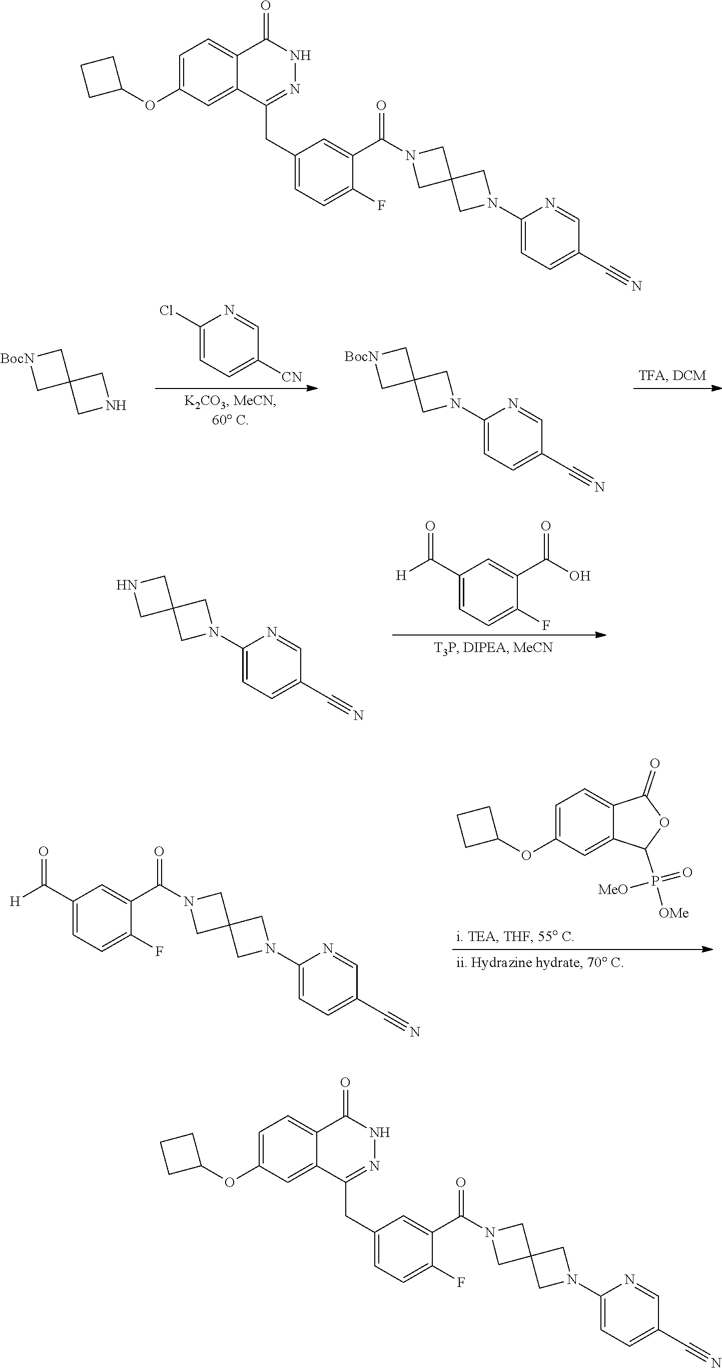

### Step 1

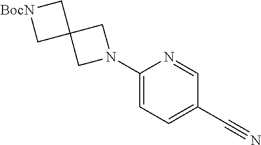 [SMILES: CC(C)(C)OC(=O)N1CC2(C1)CN(c1ccc(C#N)cn1)C2]

### tert-butyl 6-(5-cyanopyridin-2-yl)-2,6-diazaspiro[3.3]heptane-2-carboxylate

A mixture of 6-chloronicotinonitrile (241 mg, 1.74 mmol), tert-butyl 2,6-diazaspiro[3.3]heptane-2-carboxylate (328 mg, 1.65 mmol) and potassium carbonate (572 mg, 4.14 mmol) in MeCN (3.00 mL) was stirred at 60° C. overnight. The reaction was diluted with H₂O (20 mL) and extracted with EtOAc (20 mL×3). The combined organic layers were washed with brine, dried over Na₂SO₄, filtered and concentrated under reduce pressure. The residue was purified with silica gel chromatography using EtOAc/Heptanes from 0% to 100% to provide tert-butyl 6-(5-cyanopyridin-2-yl)-2,6-diazaspiro[3.3]heptane-2-carboxylate (250 mg, 50%) as a white solid. ¹H NMR (400 MHz, CDCl3) δ 8.35 (d, J=1.7 Hz, 1H), 7.57 (dd, J=8.8, 2.2 Hz, 1H), 6.22 (d, J=8.8 Hz, 1H), 4.21 (s, 4H), 4.11 (s, 4H), 1.42 (s, 9H).

### Step 2

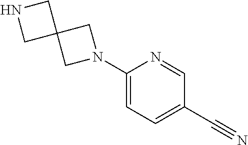 [SMILES: N#Cc1ccc(N2CC3(CNC3)C2)nc1]

### 6-(2,6-diazaspiro[3.3]heptan-2-yl)nicotinonitrile

To a solution of tert-butyl 6-(5-cyanopyridin-2-yl)-2,6-diazaspiro[3.3]heptane-2-carboxylate (250 mg, 0.832 mmol) in dichloromethane (1.5 mL) was added trifluoroacetic acid (1.5 mL). The resulting mixture was stirred at room temperature for 1 hour. The reaction mixture was concentrated under reduced pressure to give 6-(2,6-diazaspiro[3.3]heptan-2-yl)nicotinonitrile (165 mg, 99%) as yellow oil. LCMS ESI m/z: 201.2, [M+H]⁺.

### Step 3

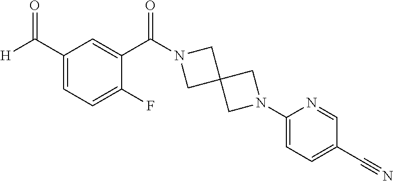 [SMILES: N#Cc1ccc(N2CC3(CN(C(=O)c4cc(C=O)ccc4F)C3)C2)nc1]

### 6-(6-(2-fluoro-5-formylbenzoyl)-2,6-diazaspiro[3.3]heptan-2-yl)nicotinonitrile

To a solution of 6-(2,6-diazaspiro[3.3]heptan-2-yl)nicotinonitrile, 2-fluoro-5-formylbenzoic acid, N,N-diisopropylethylamine (321 μL, 1.83 mmol) in MeCN (3.00 mL) was added 1-propylphosphonic acid cyclic anhydride (546 μL, 0.917 mmol). The reaction mixture was stirred at room temperature for 5 hours. The reaction was diluted with H₂O (20 mL) and extracted with EtOAc (20 mL×3). The combined organic layers were washed with brine, dried over Na₂SO₄, filtered and concentrated under reduced pressure. The residue was purified by silica gel column chromatography using EtOAc/heptanes from 0% to 100% to provide 6-(6-(2-fluoro-5-formylbenzoyl)-2,6-diazaspiro[3.3]heptan-2-yl)nicotinonitrile (234 mg, 80%) as a white solid. LCMS ESI m/z: 351.2, [M+H]⁺.

### Step 4

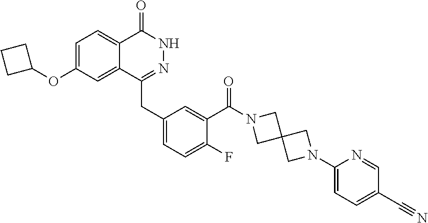 [SMILES: N#Cc1ccc(N2CC3(CN(C(=O)c4cc(Cc5n[nH]c(=O)c6ccc(OC7CCC7)cc56)ccc4F)C3)C2)nc1]

### 6-(6-(5-((7-cyclobutoxy-4-oxo-3,4-dihydrophthalazin-1-yl)methyl)-2-fluorobenzoyl)-2,6-diazaspiro[3.3]heptan-2-yl)nicotinonitrile

Following the procedure described in Example 151, and making non-critical variation as required to replace a with 6-(6-(2-fluoro-5-formylbenzoyl)-2,6-diazaspiro[3.3]heptan-2-yl)nicotinonitrile, the product of 6-(6-(5-((7-cyclobutoxy-4-oxo-3,4-dihydrophthalazin-1-yl)methyl)-2-fluorobenzoyl)-2,6-diazaspiro[3.3]heptan-2-yl)nicotinonitrile was obtained as a white solid (59 mg, 23%). LCMS EST m/z: 551.4 [M+H]⁺. ¹H NMR (400 MHz, DMSO-d₆) δ 12.48 (s, 1H), 8.45 (dd, J=2.2, 0.6 Hz, 1H), 8.15 (d, J=8.8 Hz, 1H), 7.82 (dd, J=8.8, 2.3 Hz, 1H), 7.51 (dd, J=6.6, 2.1 Hz, 1H), 7.48-7.40 (m, 1H), 7.30 (dd, J=8.8, 2.3 Hz, 1H), 7.24 (dd, J=9.8, 8.7 Hz, 1H), 7.12 (d, J=2.3 Hz, 1H), 6.43 (d, J=8.8 Hz, 1H), 4.95-4.78 (m, 1H), 4.30 (s, 2H), 4.23 (t, J=4.7 Hz, 4H), 4.18 (d, J=7.7 Hz, 4H), 2.37 (dt. J=9.2, 8.0 Hz, 2H), 2.07-1.92 (m, 2H), 1.84-1.57 (m, 2H).

### Synthesis of Example 149: 6-(2-(((7-cyclobutoxy-4-oxo-3,4-dihydrophthalazin-1-yl)methyl)-2-fluorobenzoyl)-2,5-diazaspiro[3.4]octan-5-yl)nicotinonitrile

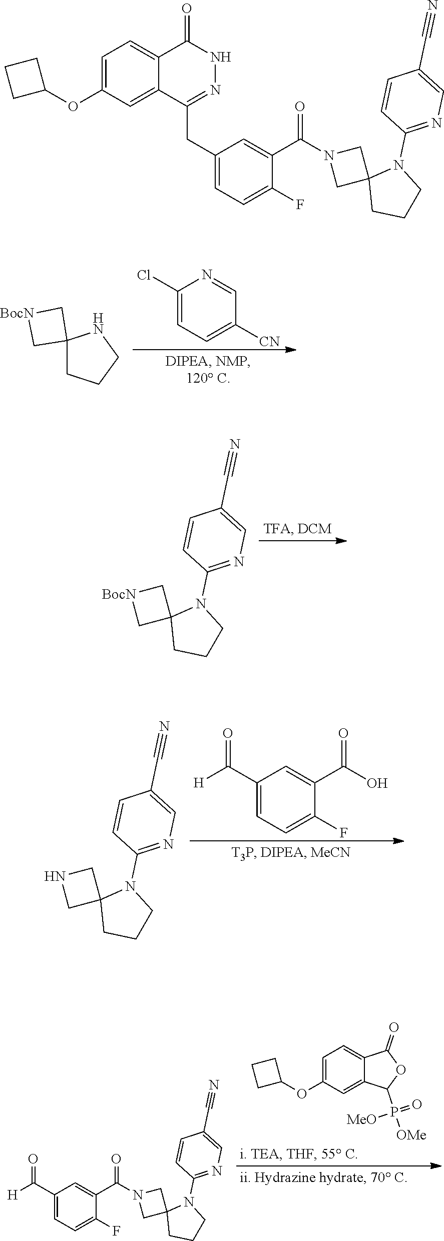

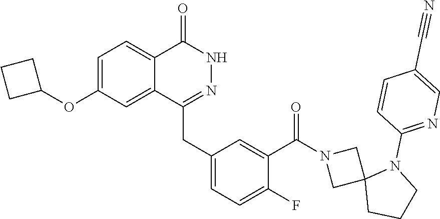 [SMILES: N#Cc1ccc(N2CCCC23CN(C(=O)c2cc(Cc4n[nH]c(=O)c5ccc(OC6CCC6)cc45)ccc2F)C3)nc1]

### Step 1

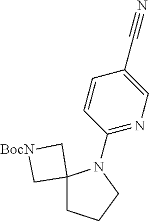 [SMILES: CC(C)(C)OC(=O)N1CC2(CCCN2c2ccc(C#N)cn2)C1]

### tert-butyl 5-(5-cyanopyridin-2-yl)-2,5-diazaspiro[3.4]octane-2-carboxylate

A solution of 6-chloronicotinonitrile (261 mg, 1.880 mmol), tert-butyl 2,5-diazaspiro[3.4]octane-2-carboxylate (400 mg, 1.880 mmol) and DIPEA (0.9 mL) in NMP (3.0 mL) was stirred at 120° C. The reaction was diluted with H₂O (20 mL) and extracted with EtOAc (20 mL×3). The combined organic layers were washed with brine, dried over Na₂SO₄, filtered and concentrated under reduced pressure. The residue was purified by silica gel column chromatography using EtOAc/heptanes from 0% to 100% to afford tert-butyl 5-(5-cyanopyridin-2-yl)-2,5-diazaspiro[3.4]octane-2-carboxylate (382 mg, 57%) as a white solid. 1H NMR (400 MHz, CDCl₃) δ 8.39 (d, J=1.8 Hz, 1H), 7.62 (dd, J=8.9, 2.3 Hz, 1H), 6.46 (d, J=8.7 Hz, 1H), 4.77 (d, J=8.3 Hz, 2H), 3.69 (d, J=8.5 Hz, 2H), 3.45 (t, J=6.6 Hz, 2H), 2.31 (t, J=6.8 Hz, 2H), 2.01-1.83 (m, 2H), 1.48 (s, 9H).

### Step 2

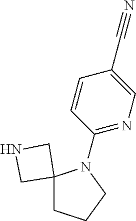 [SMILES: N#Cc1ccc(N2CCCC23CNC3)nc1]

### 6-(2,5-diazaspiro[3.4]octan-5-yl)nicotinonitrile

Following the procedure described in Example 148, and making non-critical variation as required to replace tert-butyl 6-(5-cyanopyridin-2-yl)-2,6-diazaspiro[3.3]heptane-2-carboxylate with tert-butyl 5-(5-cyanopyridin-2-yl)-2,5-diazaspiro[3.4]octane-2-carboxylate, the crude product of 6-(2,5-diazaspiro[3.4]octan-5-yl)nicotinonitrile was obtained as a yellow oil (172 mg, 99%). LCMS ESI m/z: 215.3, [M+H]⁺.

### Step 3

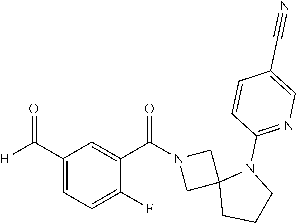 [SMILES: N#Cc1ccc(N2CCCC23CN(C(=O)c2cc(C=O)ccc2F)C3)nc1]

### 6-(2-(2-fluoro-5-formylbenzoyl)-2,5-diazaspiro[3.4]octan-5-yl)nicotinonitrile

Following the procedure described in Example 148, and making non-critical variation as required to replace 6-(2,6-diazaspiro[3.3]heptan-2-yl)nicotinonitrile with 6-(2,5-diazaspiro[3.4]octan-5-yl)nicotinonitrile, 6-(2-(2-fluoro-5-formylbenzoyl)-2,5-diazaspiro[3.4]octan-5-yl)nicotinonitrile was obtained as a white solid (172 mg, 99%). LCMS ESI m/z: 365.3, [M+H]⁺.

### Step 4

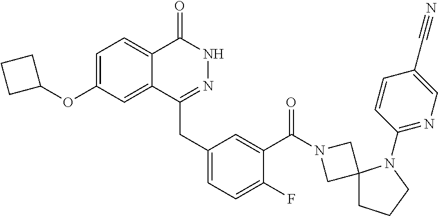 [SMILES: N#Cc1ccc(N2CCCC23CN(C(=O)c2cc(Cc4n[nH]c(=O)c5ccc(OC6CCC6)cc45)ccc2F)C3)nc1]

### 6-(2-(5-((7-cyclobutoxy-4-oxo-3,4-dihydrophthalazin-1-yl)methyl)-2-fluorobenzoyl)-2,5-diazaspiro[3.4]octan-5-yl)nicotinonitrile

Following the procedure described in Example 148, and making non-critical variation as required to replace 6-(6-(2-fluoro-5-formylbenzoyl)-2,6-diazaspiro[3.3]heptan-2-yl)nicotinonitrile with 6-(2-(2-fluoro-5-formylbenzoyl)-2,5-diazaspiro[3.4]octan-5-yl)nicotinonitrile, 6-(2-(5-((7-cyclobutoxy-4-oxo-3,4-dihydrophthalazin-1-yl)methyl)-2-fluorobenzoyl)-2,5-diazaspiro[3.4]octan-5-yl)nicotinonitrile was obtained as a white solid (351 mg, 79%). LCMS ESI m/z: 565.3. [M+H]⁺. ¹H NMR (400 MHz, DMSO-d₆) δ 12.70-12.19 (m, 1H), 8.77-8.38 (m, 1H), 8.28-8.05 (m, 1H), 8.02-7.80 (m, 1H), 7.55-7.33 (m, 2H), 7.33-7.16 (m, 2H), 7.16-6.98 (m, 1H), 6.98-6.70 (m, 1H), 4.95-4.59 (m, 1H), 4.41-4.12 (m, 2H), 3.85 (d, J=48.3 Hz, 1H), 3.65-3.32 (m, 2H), 3.20-2.85 (m, 2H), 2.34 (d, J=4.4 Hz, 1H), 2.00-1.87 (m, 2H), 1.77-1.56 (m, 2H), 1.35-1.17 (m, 2H), 0.77 (ddd, J=26.3, 13.2, 6.5 Hz, 1H), 0.38 (ddd, J=15.0, 10.2, 4.9 Hz, 1H).

### Synthesis of Example 150: 6-(5-(5-((7-cyclobutoxy-4-oxo-3,4-dihydrophthalazin-1-yl)methyl)-2-fluorobenzoyl)-2,5-diazabicyclo[4.1.0]heptan-2-yl)nicotinonitrile

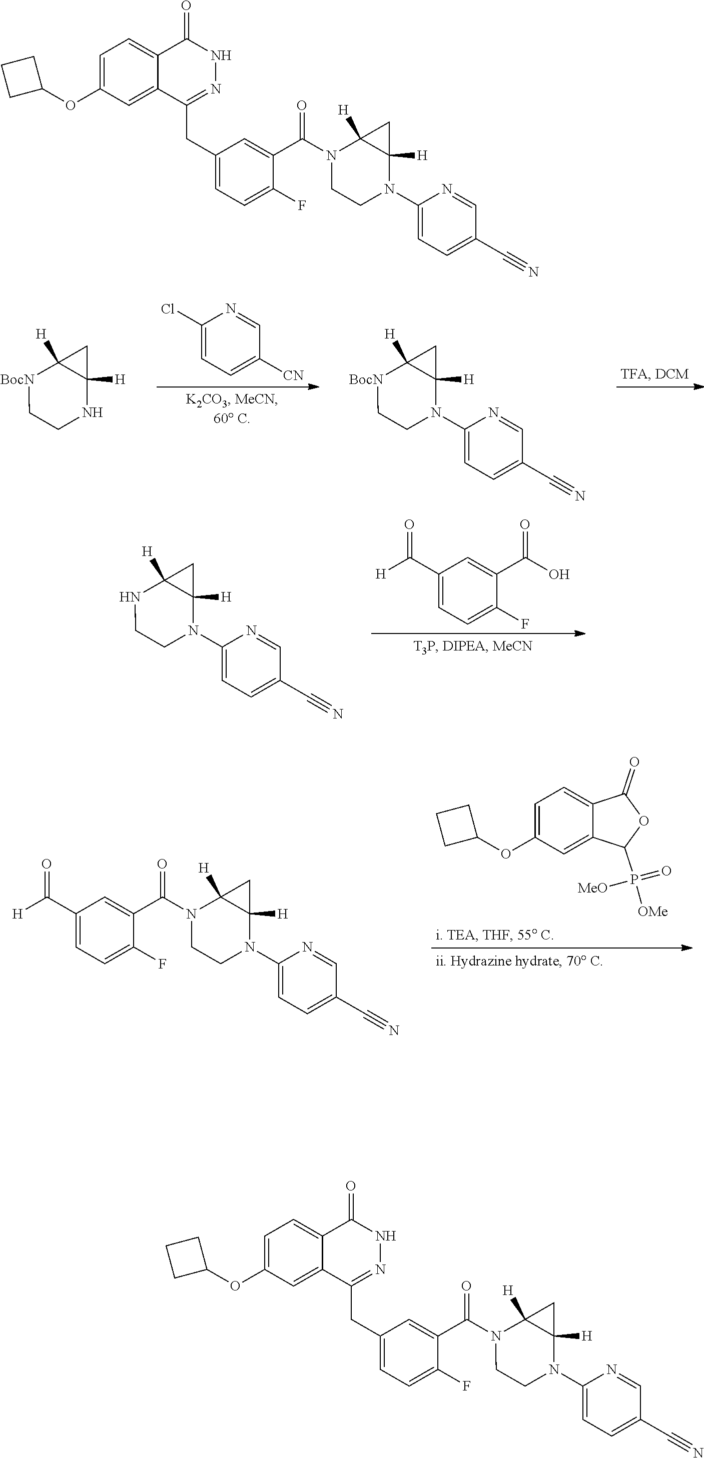

### Step 1

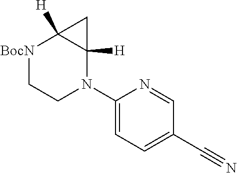 [SMILES: CC(C)(C)OC(=O)N1CCN(c2ccc(C#N)cn2)[C@H]2C[C@H]21]

### tert-butyl 5-(5-cyanopyridin-2-yl)-2,5-diazabicyclo[4.1.0]heptane-carboxylate

Following the procedure described in Example 148, and making non-critical variation as required to replace tert-butyl 2,6-diazaspiro[3.3]heptane-2-carboxylate with, tert-butyl 5-(5-cyanopyridin-2-yl)-2,5-diazabicyclo[4.1.0]heptane-2-carboxylate was obtained as a white solid (330 mg, 66%). LCMS ESI m/z: 301.3, [M+H]⁺.

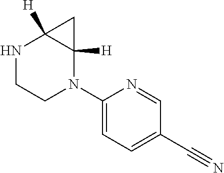 [SMILES: N#Cc1ccc(N2CCN[C@@H]3C[C@@H]32)nc1]

### 6-(2,5-diazabicyclo[4.1.0]heptan-2-yl)nicotinonitrile

Following the procedure described in Example 148, and making non-critical variation as required to replace tert-butyl 6-(5-cyanopyridin-2-yl)-2,6-diazaspiro[3.3]heptane-2-carboxylate with tert-butyl 5-(5-cyanopyridin-2-yl)-2,5-diazabicyclo[4.1.0]heptane-2-carboxylate, the crude product of 6-(2,5-diazabicyclo[4.1.0]heptan-2-yl)nicotinonitrile was obtained as a yellow oil (218 mg, 99%/0). LCMS ESI m/z: 301.3, [M+H]⁺.

### Step 3

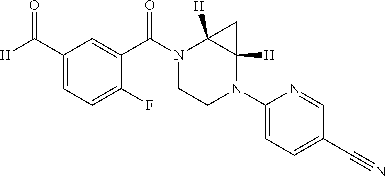 [SMILES: N#Cc1ccc(N2CCN(C(=O)c3cc(C=O)ccc3F)[C@@H]3C[C@@H]32)nc1]

### 6-(5-(2-fluoro-5-formylbenzoyl)-2,5-diazabicyclo[4.1.0]heptan-2-yl)nicotinonitrile

Following the procedure described in Example 148, and making non-critical variation as required to replace 6-(2,6-diazaspiro[3.3]heptan-2-yl)nicotinonitrile with 6-(2,5-diazabicyclo[4.1.0]heptan-2-yl)nicotinonitrile, 6-(5-(2-fluoro-5-formylbenzoyl)-2,5-diazabicyclo[4.1.0]heptan-2-yl)nicotinonitrile was obtained as a white solid (280 mg, 73%). LCMS ESI m/z: 351.3, [M+H]⁺.

### Step 4

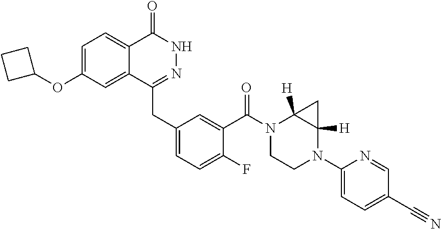 [SMILES: N#Cc1ccc(N2CCN(C(=O)c3cc(Cc4n[nH]c(=O)c5ccc(OC6CCC6)cc45)ccc3F)[C@@H]3C[C@@H]32)nc1]

### 6-(5-(5-((7-cyclobutoxy-4-oxo-3,4-dihydrophthalazin-1-yl)methyl)-2-fluorobenzoyl)-2,5-diazabicyclo[4.1.0]heptan-2-yl)nicotinonitrile

Following the procedure described in Example 148, and making non-critical variation as required to replace 6-(6-(2-fluoro-5-formylbenzoyl)-2,6-diazaspiro[3.3]heptan-2-yl)nicotinonitrile with 6-(5-(2-fluoro-5-formylbenzoyl)-2,5-diazabicyclo[4.1.0]heptan-2-yl)nicotinonitrile, 6-(5-(5-((7-cyclobutoxy-4-oxo-3,4-dihydrophthalazin-1-yl)methyl)-2-fluorobenzoyl)-2,5-diazabicyclo[4.1.0]heptan-2-yl)nicotinonitrile was obtained as a white solid (85 mg, 29%). LCMS ESI m/z: 551.3, [M+H]⁺. ¹H NMR (400 MHz, DMSO-d₆) δ 12.70-12.19 (m, 1H), 8.77-8.38 (m, 1H), 8.28-8.05 (m, 1H), 8.02-7.80 (m, 1H), 7.55-7.33 (m, 2H), 7.33-7.16 (m, 2H), 7.16-6.98 (m, 1H), 6.98-6.70 (m, 1H), 4.95-4.59 (m, 1H), 4.41-4.12 (m, 2H), 3.85 (d, J=48.3 Hz, 1H), 3.65-3.32 (m, 2H), 3.20-2.85 (m, 2H), 2.34 (d, J=4.4 Hz, 1H), 2.00-1.87 (m, 2H), 1.77-1.56 (m, 2H), 1.35-1.17 (m, 2H), 0.77 (ddd, J=26.3, 13.2, 6.5 Hz, 1H), 0.38 (ddd, J=15.0, 10.2, 4.9 Hz, 1H).

### Synthesis of Example 151: 6-(7-(5-((7-cyclobutoxy-4-oxo-3,4-dihydrophthalazin-1-yl)methyl)-2-fluorobenzoyl)-2,7-diazaspiro[4.4]nonan-2-yl)nicotinonitrile formate salt

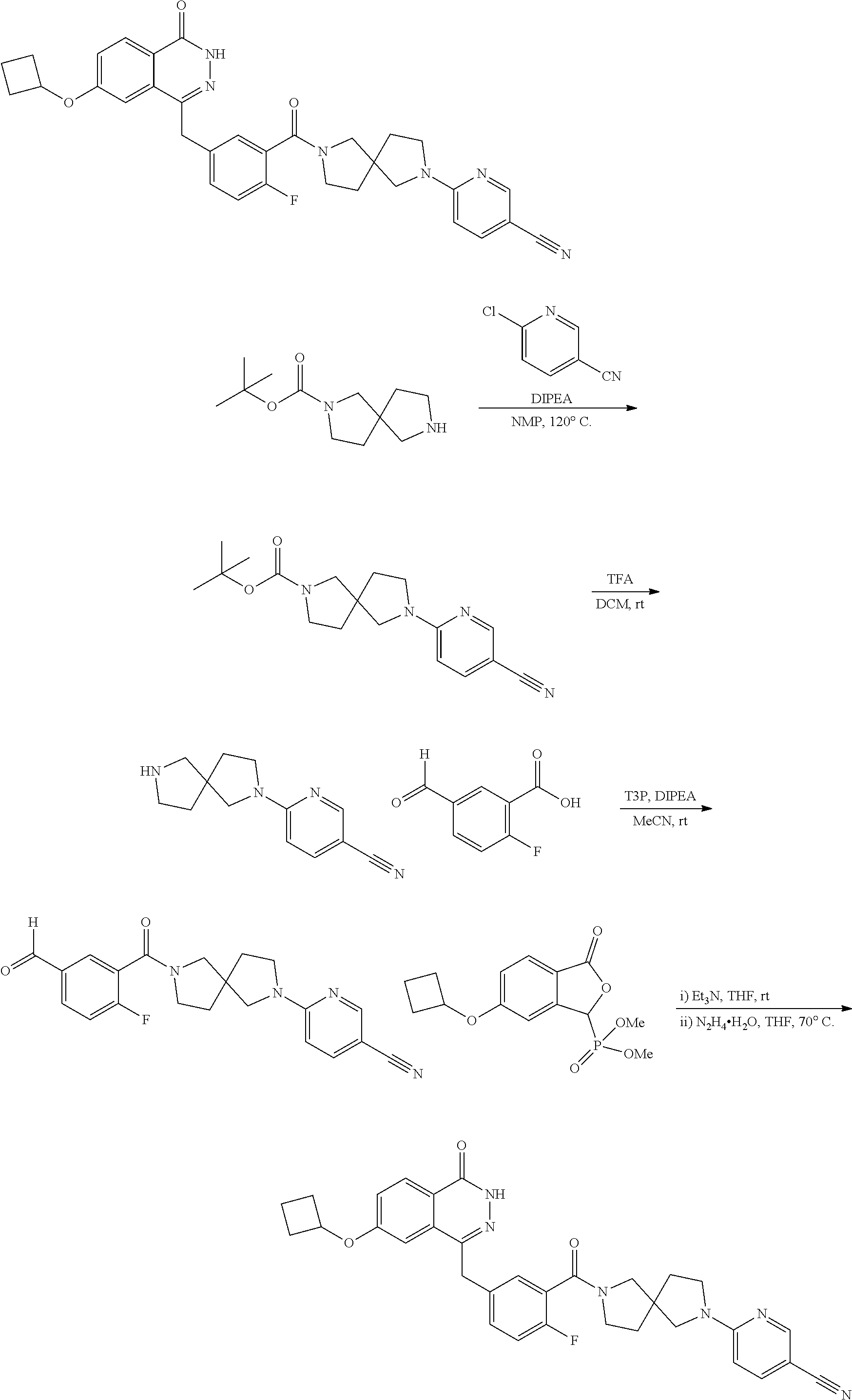

### Step 1

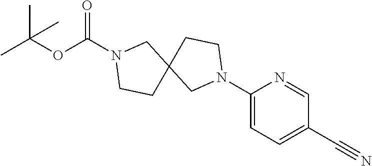 [SMILES: CC(C)(C)OC(=O)N1CCC2(CCN(c3ccc(C#N)cn3)C2)C1]

### tert-butyl 7-(5-cyanopyridin-2-yl)-2,7-diazaspiro[4.4]nonane-2-carboxylate

A mixture of 6-chloronicotinonitrile (77 mg, 0.55 mmol), 6-(2,7-diazaspiro[4.4]nonan-2-yl)nicotinonitrile (130 mg, 0.55 mmol) and DIPEA (0.20 mL, 1.14 mmol) in NMP (2 mL) was stirred at 120° C. for 24 hours. The mixture was cooled to rt, diluted with H₂O (50 mL) and extracted with EtOAc (20 mL×3). The combined organic layer was washed with brine, dried over anhydrous Na₂SO₄, filtered and concentrated in vacuo. The residue was purified by silica gel chromatography (EtOAc/Heptanes=0 to 50%) to give tert-butyl 7-(5-cyanopyridin-2-yl)-2,7-diazaspiro[4.4]nonane-2-carboxylate (178 mg, 0.54 mmol, 99%) as a clear oil. LCMS EST m/z: 329.3 [M+H]⁺.

### Step 2

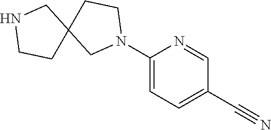 [SMILES: N#Cc1ccc(N2CCC3(CCNC3)C2)nc1]

### 6-(2,7-diazaspiro[4.4]nonan-2-yl)nicotinonitrile

A mixture of tert-butyl 7-(5-cyanopyridin-2-yl)-2,7-diazaspiro[4.4]nonane-2-carboxylate (178 mg, 0.54 mmol). TFA (1.8 mL) in DCM (1.8 mL) was stirred at rt for 1 hour. The mixture concentrated in vacuo to give 6-(2,7-diazaspiro[4.4]nonan-2-yl)nicotinonitrile (124 mg, 0.54 mmol, 99%) as a clear oil that was carried forward without further purification. LCMS ESI m/z: 229.3 [M+H]⁺.

### Step 3

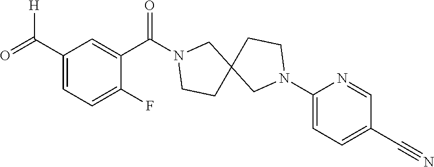 [SMILES: N#Cc1ccc(N2CCC3(CCN(C(=O)c4cc(C=O)ccc4F)C3)C2)nc1]

### 6-(7-(2-fluoro-5-formylbenzoyl)-2,7-diazaspiro[4.4]nonan-2-yl)nicotinonitrile

A mixture of 6-(2,7-diazaspiro[4.4]nonan-2-yl)nicotinonitrile (124 mg, 0.54 mmol), 2-fluoro-5-formylbenzoic acid (91 mg, 0.54 mmol), T3P (0.44 mL, 0.60 mmol), and DIPEA (0.48 mL, 2.72 mmol) in MeCN (2.2 mL) was stirred at rt for 24 hours. The mixture was diluted with H₂O (20 mL) and extracted with EtOAc (20 mL×3). The combined organic layer was washed with brine, dried over anhydrous Na₂SO₄, filtered and concentrated in vacuo. The residue was purified by silica gel chromatography (EtOAc/Heptanes=0 to 100%) to give 6-(7-(2-fluoro-5-formylbenzoyl)-2,7-diazaspiro[4.4]nonan-2-yl)nicotinonitrile (21 mg, 0.06 mmol, 8%) as a clear oil. LCMS EST m/z: 379.3 [M+H]⁺.

### Step 4

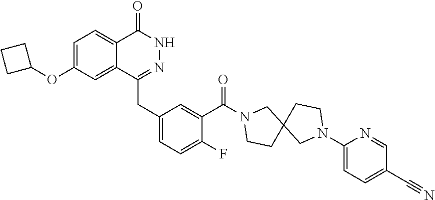 [SMILES: N#Cc1ccc(N2CCC3(CCN(C(=O)c4cc(Cc5n[nH]c(=O)c6ccc(OC7CCC7)cc56)ccc4F)C3)C2)nc1]

### 6-(7-(5-((7-cyclobutoxy-4-oxo-3,4-dihydrophthalazin-1-yl)methyl)-2-fluorobenzoyl)-2,7-diazaspiro[4.4]nonan-2-yl)nicotinonitrile formate salt

A mixture of dimethyl (6-cyclobutoxy-3-oxo-1,3-dihydroisobenzofuran-1-yl)phosphonate (17 mg, 0.056 mmol), 6-(7-(2-fluoro-5-formylbenzoyl)-2,7-diazaspiro[4.4]nonan-2-yl)nicotinonitrile (21 mg, 0.056 mmol) and Et₃N (0.02 mL, 0.17 mmol) in THF (0.6 mL) was stirred at rt for 18 hours. To the reaction mixture was added N₂H₄·H₂O (0.01 mL, 0.32 mmol), then stirred at 70° C. for 1 hour. The reaction mixture was concentrated in vacuo and the resulting crude was subjected to reverse phase column chromatography (MeCN:AMF=0 to 100%) to afford 6-(7-(5-((7-cyclobutoxy-4-oxo-3,4-dihydrophthalazin-1-yl)methyl)-2-fluorobenzoyl)-2,7-diazaspiro[4.4]nonan-2-yl)nicotinonitrile formate salt as a white solid (2 mg, 0.004 mmol, 7/o). LCMS ESI m/z: 579.5 [M+H]⁺. ¹H NMR (400 MHz, DMSO-d₆) δ 12.46 (s, 1H), 8.46 (d, J=8.2 Hz, 1H), 8.24 (s, 1H), 8.14 (dd, J=8.7, 7.8 Hz, 1H), 7.82 (ddd, J=11.3, 9.0, 2.1 Hz, 1H), 7.47-7.36 (m, 2H), 7.32-7.27 (m, 1H), 7.26-7.20 (m, 1H), 7.07 (dd, J=27.6, 2.2 Hz, 1H), 6.57-6.49 (m, 1H), 4.86-4.77 (m, 1H), 4.29 (d, J=10.2 Hz, 2H), 3.63-3.50 (m, 2H), 3.49 (d, J=4.3 Hz, 1H), 3.30-3.22 (m, 3H), 3.11 (s, 1H), 2.41-2.32 (m, 4H), 2.14-1.77 (m, 6H), 1.77-1.59 (m, 1H).

### Synthesis of Example 152: 6-(7-(5-((7-Cyclobutoxy-4-oxo-3,4-dihydrophthalazin-1-yl)methyl)-2-fluorobenzoyl)-1,7-diazaspiro[3,5]nonan-1-yl)nicotinonitrile

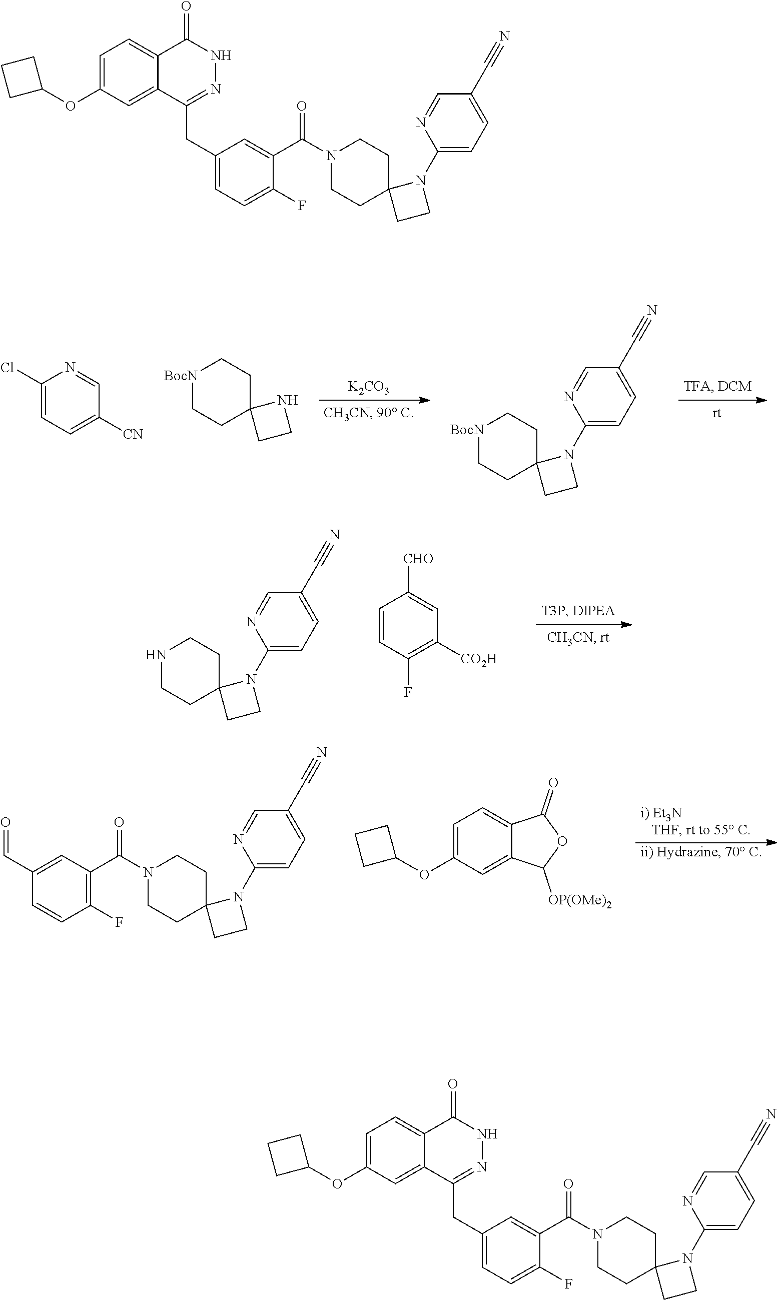

### Step 1

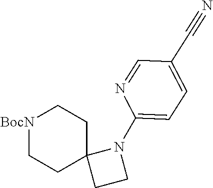 [SMILES: CC(C)(C)OC(=O)N1CCC2(CC1)CCN2c1ccc(C#N)cn1]

### tert-Butyl 1-(5-cyanopyridin-2-yl)-1,7-diazaspiro[3.5]nonane-7-carboxylate

The solution of 6-Chloronicotinonitrile (148 mg, 1.07 mmol), tert-Butyl 1,7-diazaspiro[3.5]nonane-7-carboxylate (250 mg, 1.07 mmol) and DIPEA (0.3 mL) in NMP (3.0 mL) was stirred o/n at 120° C. The reaction was diluted with H₂O (20 mL) and extracted with EtOAc (20 mL×3). The combined organic layers were washed with brine, dried over Na₂SO₄, filtered and concentrated in vacuo. The residue was purified by silica gel chromatography (EtOAc/heptanes=0 to 100%) to give tert-Butyl 1-(5-cyanopyridin-2-yl)-1,7-diazaspiro[3.5]nonane-7-carboxylate (271 mg, 77% yield). LCMS ESI 329.3 [M+H]⁺.

### Step 2

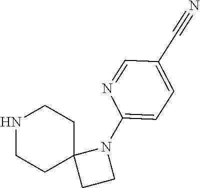 [SMILES: N#Cc1ccc(N2CCC23CCNCC3)nc1]

### 6-(1,7-diazaspiro[3,5]nonan-1-yl)nicotinonitrile

The solution of tert-Butyl 1-(5-cyanopyridin-2-yl)-1,7-diazaspiro[3.5]nonane-7-carboxylate (271 mg, 824 umol). TFA (2 mL) and CH₂Cl₂ (2 mL) was stirred at rt for 1 h. The reaction mixture was concentrated in vacuo to obtain 6-(1,7-diazaspiro[3.5]nonan-1-yl)nicotinonitrile (172 mg, 99% yield). LCMS EST 229.3 [M+H]⁺.

### Step 3

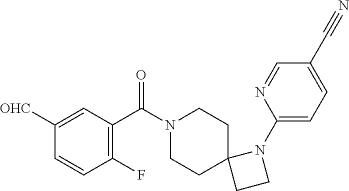 [SMILES: N#Cc1ccc(N2CCC23CCN(C(=O)c2cc(C=O)ccc2F)CC3)nc1]

### 6-(7-(2-Fluoro-5-formylbenzoyl)-1,7-diazaspiro[3.5]nonan-1-yl)nicotinonitrile

To a solution of 2-fluoro-5-formylbenzoic acid (127 mg, 753 umol). N,N-Diisopropylethylamine (290 uL, 1.66 mmol), 1-Propylphosphonic acid cyclic anhydride (493 uL, 829 umol) in MeCN (3.00 mL) was added 6-(1,7-diazaspiro[3.5]nonan-1-yl)nicotinonitrile (172 mg, 753 umol). The reaction mixture was stirred o/n at rt. The reaction was diluted with H₂O (20 mL) and extracted with EtOAc (20 mL×3). The combined organic layers were washed with brine, dried over Na₂SO₄, filtered and concentrated in vacuo. The residue was purified by silica gel chromatography (EtOAc/heptanes=0 to 100%) to give 6-(7-(2-fluoro-5-formylbenzoyl)-1,7-diazaspiro[3.5]nonan-1-yl)nicotinonitrile (246 mg, 86%). LCMS EST 379.3 [M+H]⁺.

### Step 4

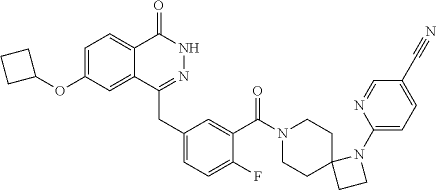 [SMILES: N#Cc1ccc(N2CCC23CCN(C(=O)c2cc(Cc4n[nH]c(=O)c5ccc(OC6CCC6)cc45)ccc2F)CC3)nc1]

### 6-(7-(5-((7-Cyclobutoxy-4-oxo-3,4-dihydrophthalazin-1-yl)methyl)-2-fluorobenzoyl)-1,7-diazaspiro[3.5]nonan-1-yl)nicotinonitrile

Following the procedure described in Example 148, and making non-critical variation as required to replace 6-(6-(2-fluoro-5-formylbenzoyl)-2,6-diazaspiro[3.3]heptan-2-yl)nicotinonitrile with 6-(7-(2-fluoro-5-formylbenzoyl)-1,7-diazaspiro[3.5]nonan-1-yl)nicotinonitrile: 6-(7-(5-((7-cyclobutoxy-4-oxo-3,4-dihydrophthalazin-1-yl)methyl)-2-fluorobenzoyl)-1,7-diazaspiro[3.5]nonan-1-yl)nicotinonitrile was obtained as a white solid (51.3 mg, 23% yield). LCMS (ESI) m/z: 579.4 [M+H]⁺. ¹H NMR (400 MHz, 100° C. DMSO-d₆) δ 12.10 (s, 1H), 8.35 (d, J=1.8 Hz, 1H), 8.16 (d, J=8.8 Hz, 1 Hz, 7.70 (dd, J=8.8, 2.3 Hz, 1H), 7.45-7.36 (m, 1H), 7.31 (dd, J=6.4, 2.0 Hz, 11), 7.26 (dd, J=8.8, 2.3 Hz, 1H), 7.19 (t, J=9.0 Hz, 1H), 7.06 (d, J=2.3 Hz, 1H), 6.30 (d, J=8.8 Hz, 1H), 5.66 (s, 2H), 4.81 (p. J=6.9 Hz, 1H), 4.30 (s, 2H), 3.94 (t, J=7.5 Hz, 2H), 2.96 (s, 3H), 2.45-2.26 (m, 6H), 2.15-1.97 (m, 2H), 1.91-1.63 (m, 4H).

### Synthesis of Example 154: 2-(4-(5-((7-cyclobutoxy-4-oxo-3,4-dihydrophthalazin-1-yl)methyl)-2-fluorobenzoyl)piperazin-1-yl)pyrimidine-5-carbonitrile

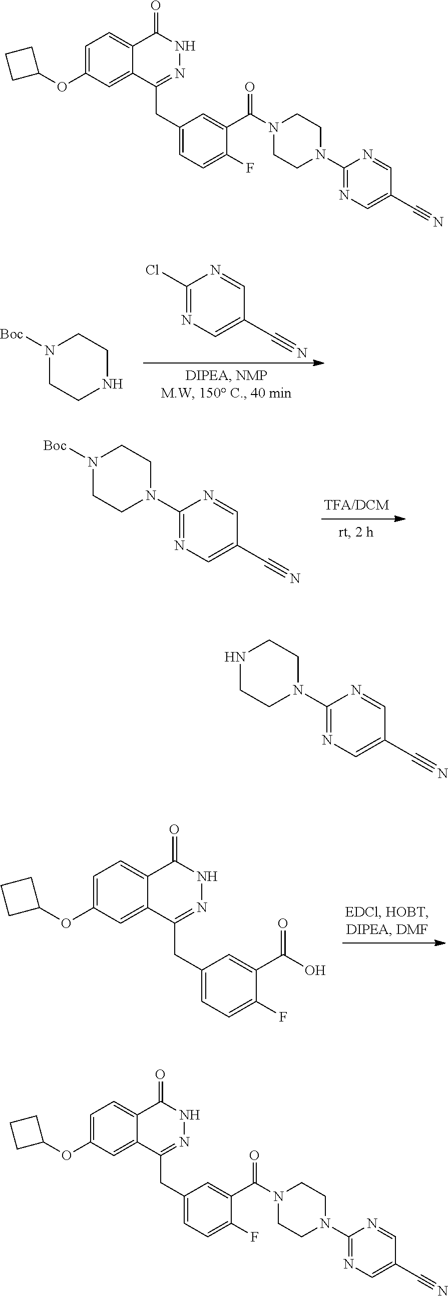

### Step 1

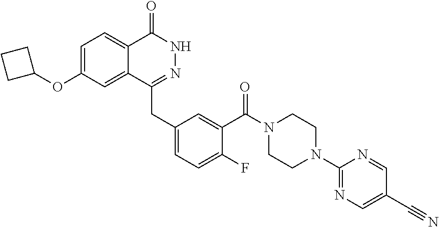 [SMILES: N#Cc1cnc(N2CCN(C(=O)c3cc(Cc4n[nH]c(=O)c5ccc(OC6CCC6)cc45)ccc3F)CC2)nc1]

### 2-(4-(5-((7-cyclobutoxy-4-oxo-3,4-dihydrophthalazin-1-yl)methyl)-2-fluorobenzoyl)piperazin-1-yl)pyrimidine-5-carbonitrile

Following the general three-step procedure above in Example 130, but starting with 2-chloropyrimidine-5-carbonitrile gave the title compound as a white solid. LCMS ESI m/z: 539.7 [M+H]⁺. ¹H NMR (400 MHz, DMSO-d₆) δ 12.44 (s, 1H), 8.79 (s, 2H), 8.16 (d, J=8.8 Hz, 1H), 7.44-7.40 (m, 2H), 7.33-7.24 (m, 2H), 7.10 (d, J=2.3 Hz, 1H), 4.85 (m, 1H), 4.31 (s, 2H), 3.93 (s, 2H), 3.75 (m, 4), 3.31-3.29 (m, 2H), 2.44-2.36 (m, 2H), 2.04-1.97 (m, 2H), 1.80-1.64 (m, 2H).

### Synthesis of Example 155: 6-Cyclobutoxy-4-(4-fluon)-3-(4-(5-(trifluoromethyl)pyridin-2-yl)piperazine-1-carbonyl)benzyl)phthalazin-1(2H)-one

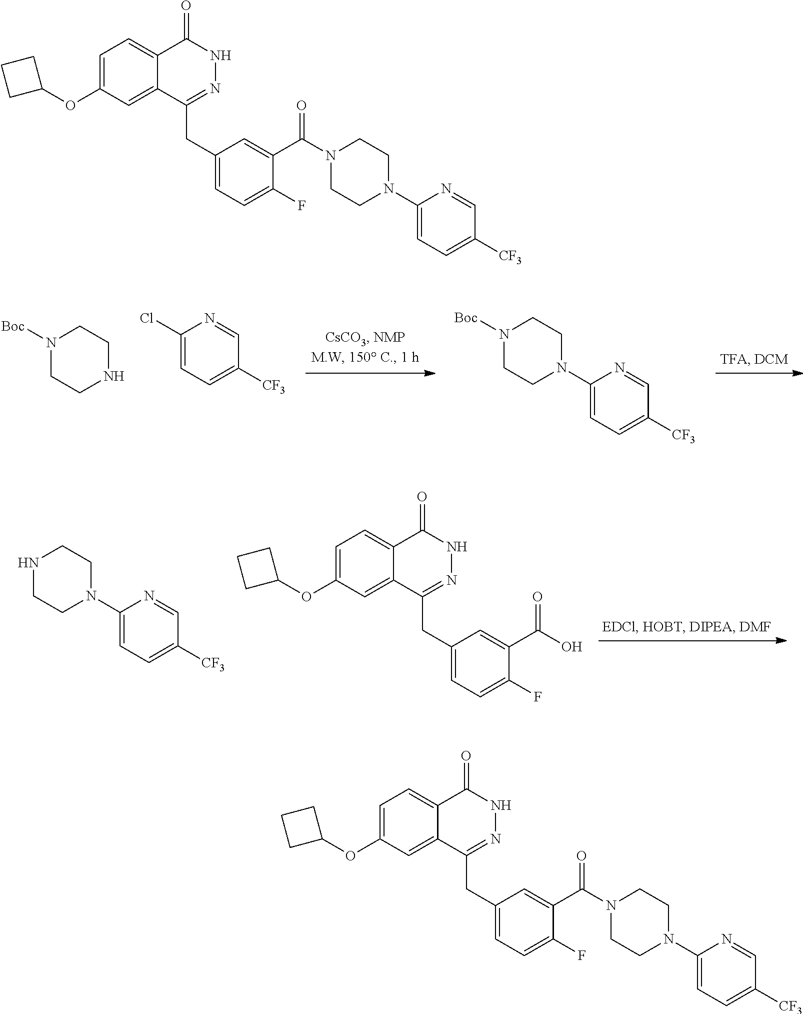

### Step 1,

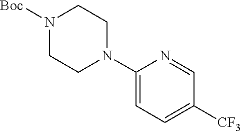 [SMILES: CC(C)(C)OC(=O)N1CCN(c2ccc(C(F)(F)F)cn2)CC1]

### tert-butyl 4-(5-(trifluoromethyl)pyridin-2-yl)piperazine-1-carboxylate

2-chloro-5-trifluoromethyl)pyridine (200 mg, 1.10 mmol, 141.14 μL), tert-butyl piperazine-1-carboxylate (271.34 mg, 1.10 mmol), Cs₂CO₃ (1.79 g, 5.51 mmol) and NMP (2 mL) were added to a 10 mL sealed tube. The resultant mixture was stirred at 150° C. for 1 hr under microwave. The mixture was poured into water (10 mL), extracted with EA (10 mL×2). The organic layer was washed with brine, dried and concentrated to a crude residue. The residue was purified by silica gel chromatography (Petroleum ether:EtOAc=10:1 to 1:1) to give tert-butyl 4-[5-(trifluoromethyl)-2-pyridyl]piperazine-1-carboxylate (180 mg, 34.0% yield) as a yellow oil. LCMS ESI m/z: 276.1 [M+H-56]⁺.

### Step 2 and 3

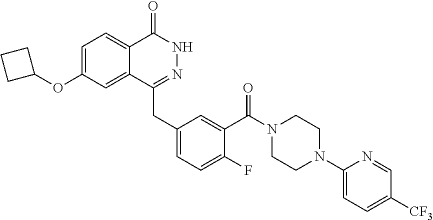 [SMILES: O=C(c1cc(Cc2n[nH]c(=O)c3ccc(OC4CCC4)cc23)ccc1F)N1CCN(c2ccc(C(F)(F)F)cn2)CC1]

### 6-cyclobutoxy-4-(4-fluoro-3-(4-(5-(trifluoromethyl)pyridin-2-yl)piperazine-1-carbonyl)benzyl)phthalazin-1(2H)-one

Following the general two-step procedure above in Example 130, but starting with tert-butyl 4-(5-(trifluoromethyl)pyridin-2-yl)piperazine-1-carboxylate gave the title compound as a white solid. LCMS ESI m/z: 581.7 [M+H]⁺. ¹H NMR (400 MHz, DMSO-D6) δ 12.44 (s, 1H), 8.43 (s, 1H), 8.16 (d, J=8.8 Hz, 1H), 7.84 (dd, J=9.1, 2.4 Hz, 1H), 7.43 (t, J=6.6 Hz, 2H), 7.34-7.23 (m, 2H), 7.10 (d, J=2.3 Hz, 1H), 6.95 (m, 1H), 4.88-4.78 (m, 1H), 4.31 (s, 2H), 3.73 (s, 4H), 3.58 (s, 2H), 3.31-3.27 (m, 2H), 2.39 (m, 2H), 2.00 (m, 2H), 1.70 (m, 2H).

### Synthesis of Example 156: 6-Cyclobutoxy-4-(4-fluoro-3-(4-(4-(trifluoromethyl)pyridin-2-yl)piperazine-1-carbonyl)benzyl)phthalazin-1(2)-one

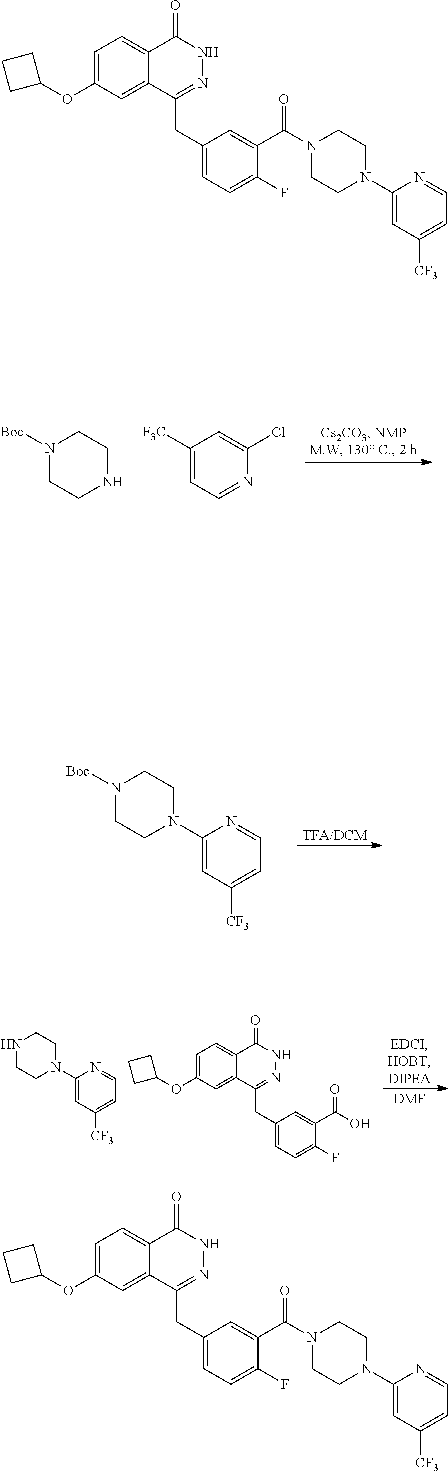

### Step 1

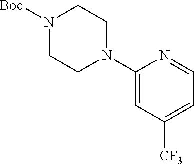 [SMILES: CC(C)(C)OC(=O)N1CCN(c2cc(C(F)(F)F)ccn2)CC1]

### tert-butyl 4-(4-(trifluoromethyl)pyridin-2-yl)piperazine-1-carboxylate

2-chloro-4-(trifluoromethyl)pyridine (100 mg, 550.83 μmol, 70.87 μL), tert-butyl piperazine-1-carboxylate (135.67 mg, 550.83 μmol), Cs₂CO₃, (538.42 mg, 1.65 mmol) and NMP (1 mL) were added to a 10 mL sealed tube. The reaction mixture was stirred under microwave at 130° C. for 2 hr. The mixture was poured into H₂O (10 mL), extracted with EA (10 mL*2). The combined organic layers were washed with brine, dried and concentrated to give a crude product tert-butyl 4-(4-(trifluoromethyl)pyridin-2-yl)piperazine-1-carboxylate as a yellow solid. LCMS ESI m/z: 332.2 [M+H]⁺.

### Step 2 and 3

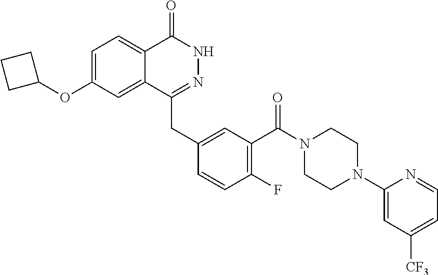 [SMILES: O=C(c1cc(Cc2n[nH]c(=O)c3ccc(OC4CCC4)cc23)ccc1F)N1CCN(c2cc(C(F)(F)F)ccn2)CC1]

### 6-cyclobutoxy-4-(4-fluoro-3-(4-(4-(trifluoromethyl)pyridin-2-yl)piperazine-1-carbonyl)benzyl)phthalazin-1(2H)-one

Following the general two-step procedure above in Example 130, but starting with tert-butyl 4-(4-(trifluoromethyl)pyridin-2-yl)piperazine-1-carboxylate gave the title compound as a white solid. LCMS EST m/z: 582.2 [M+H]⁺. ¹H NMR (4100 MHz, DMSO-D6) δ 12.45 (s, 1H), 8.35 (d, J=5.1 Hz 1H), 8.16 (d, J=8.8 Hz, 1H), 7.46-7.39 (m, 2H), 7.29 (m, 2H), 7.13-7.09 (m, 2H), 6.94 (d, J=5.2 Hz, 1H), 4.89-4.81 (m, 1H), 4.31 (s, 2H), 3.72 (d, J=7.5 Hz, 4H), 3.55 (s, 2H), 3.30 (d, J=4.9 Hz, 2H), 2.40 (m, 2H), 2.08-1.97 (m, 2H), 1.83-1.65 (m, 2H).

### Synthesis of Example 157: 6-cyclobutoxy-4-(3-(4-(5-(difluoromethyl)pyrimidin-2-yl)piperazine-1-carbonyl 4-fluorobenzyl)phthalazin-1(2H)-one

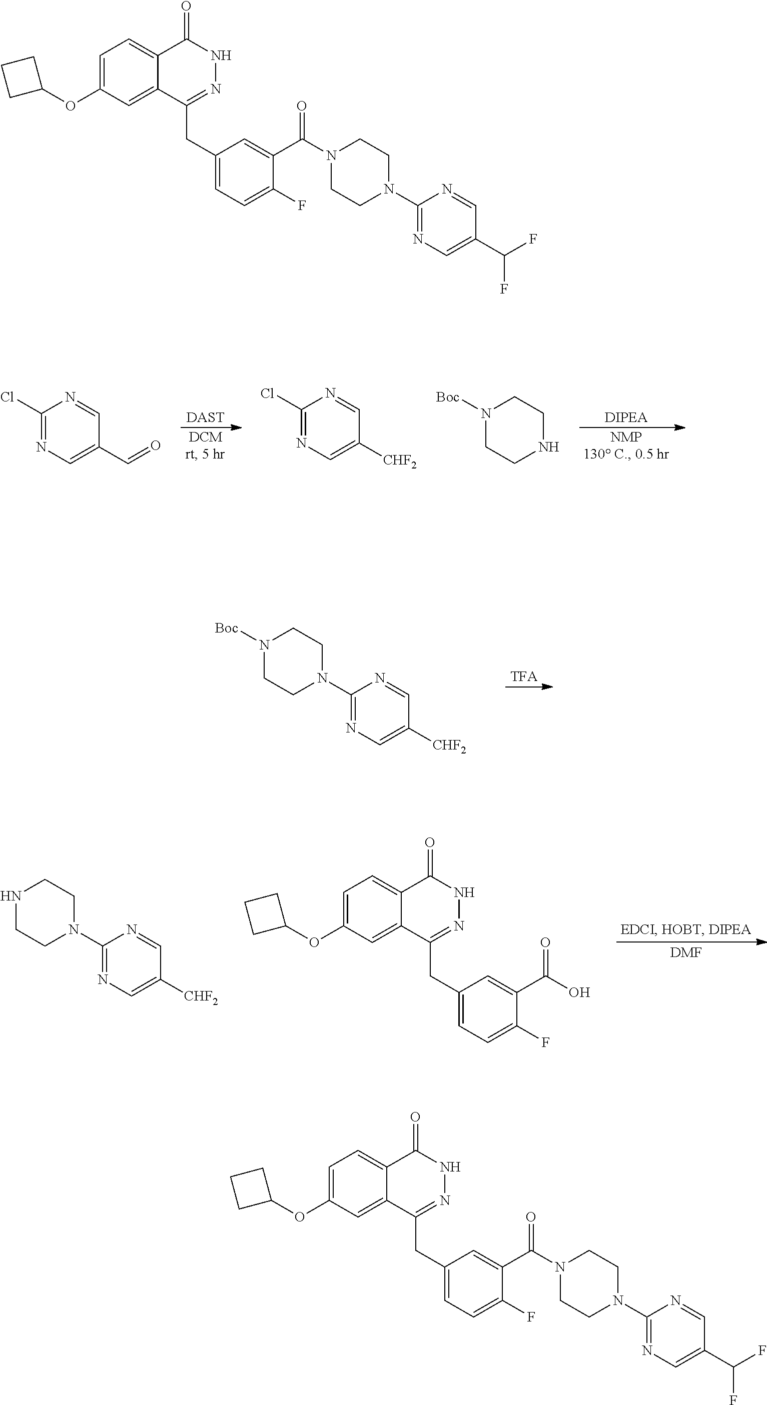

### Step 1,

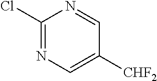

### 2-Chloro-5-(difluoromethyl)pyrimidine

To a solution of 2-chloropyrimidine-5-carbaldehyde (200 mg, 1.40 mmol) in DCM (5 mL) was added DAST (2.26 g, 14.03 mmol, 1.85 mL), and then the mixture was stirred at rt for 5 hr. The reaction mixture was poured into sat. NaHCO₃ (20 mL), extracted with DCM (10 mL). The organic layer was washed with brine, dried and concentrated to give 2-chloro-5-(difluoromethyl)pyrimidine (235 mg, crude) as a yellow oil. ¹H NMR (400 MHz, CDCl₃) δ 8.79 (s, 2H), 6.78 (t, J=55.1 Hz, 1H).

### Step 2, 3 and 4

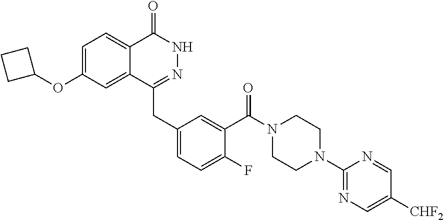 [SMILES: O=C(c1cc(Cc2n[nH]c(=O)c3ccc(OC4CCC4)cc23)ccc1F)N1CCN(c2ncc(C(F)F)cn2)CC1]

### 6-Cyclobutoxy-4-(3-(4-(5-(difluoromethyl)pyrimidin-2-yl)piperazine-1-carbonyl)-4-fluorobenzyl)phthalazin-1(2H)-one

Following the general three-step procedure above in Example 130, but starting with 2-chloro-5-(difluoromethyl)pyrimidine gave the title compound as a white solid. LCMS ESI m/z: 564.8 [M+H]⁺. ¹H NMR (400 MHz, DMSO-d₆) δ 12.45 (s, 1H), 8.58 (s, 2H), 8.16 (d, J=8.8 Hz, 1H), 7.45-7.38 (m, 2H), 7.34-7.22 (m, 2H), 7.13-6.83 (m, 2H), 4.90-4.76 (m, 1H), 4.31 (s, 2H), 3.89 (s, 2H), 3.73 (s, 4H), 3.30 (s, 2H), 2.45-2.35 (m, 2H), 2.07-1.95 (m, 2H), 1.84-1.61 (m, 2H).

### Synthesis of Example 158: 6-cyclobutoxy-4-(4-fluoro-3-(4-(6-(trifluoromethyl)pyridazin-3-yl)piperazine-1-carbonyl)benzyl)phthalazin-1(2H)-one

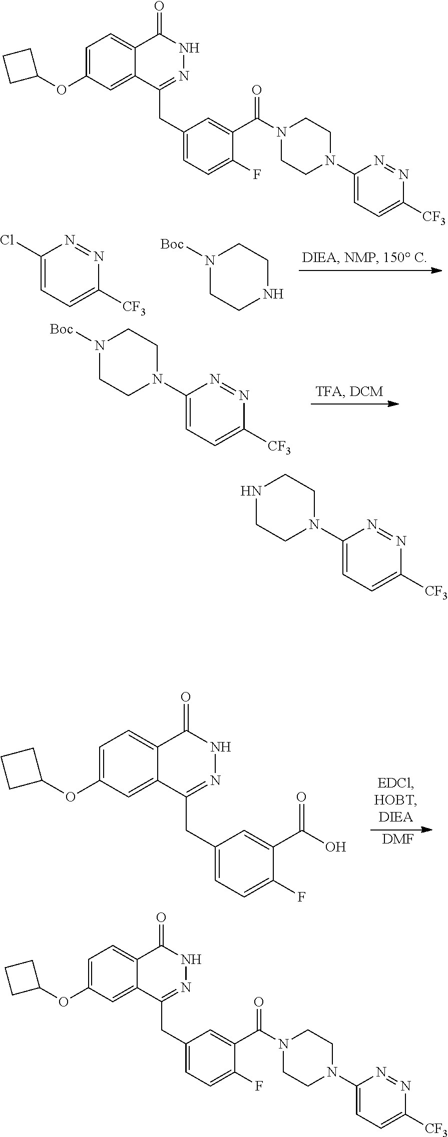

### Step 1, 2 and 3

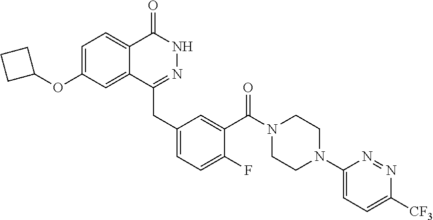 [SMILES: O=C(c1cc(Cc2n[nH]c(=O)c3ccc(OC4CCC4)cc23)ccc1F)N1CCN(c2ccc(C(F)(F)F)nn2)CC1]

### 6-Cyclobutoxy-4-(4-fluoro-3-(4-(6-(trifluoromethyl)pyridazin-3-yl)piperazine-1-carbonyl)benzyl)phthalazin-1(2H)-one

Following the general three-step procedure above in Example 130, but starting with 3-chloro-6-(trifluoromethyl)pyridazine gave the title compound as a white solid. LCMS ESI m/z: 582.7 [M+H]⁺. ¹H NMR (400 MHz, CDCl₃) δ 8.37 (d, J=8.8 Hz, 1H), 7.54 (d, J=14.2 Hz, 1H), 7.35 (dd, J=11.7, 5.4 Hz, 2H), 7.26 (s, 1H), 7.24 (s, 1H), 709 (t, J=8.7 Hz, 1H), 7.01-6.93 (m, 2H), 4.74-4.67 (m, 1H), 4.27 (s, 2H), 3.95 (s, 2H), 3.84 (t, J=4.8 Hz, 4H), 3.51 (s, 2H), 2.44 (s, 2H), 2.23-2.12 (m, 2H), 1.91 (m, 1H), 1.82-1.73 (m, 1H).

### Synthesis of Example 159: 6-Cyclobutoxy-4-(4-fluoro-3-(4-(6-(trifluoromethyl)pyridin-3-yl)piperazine-1-carbonyl)benzyl)phthalazin-1(2H)-one

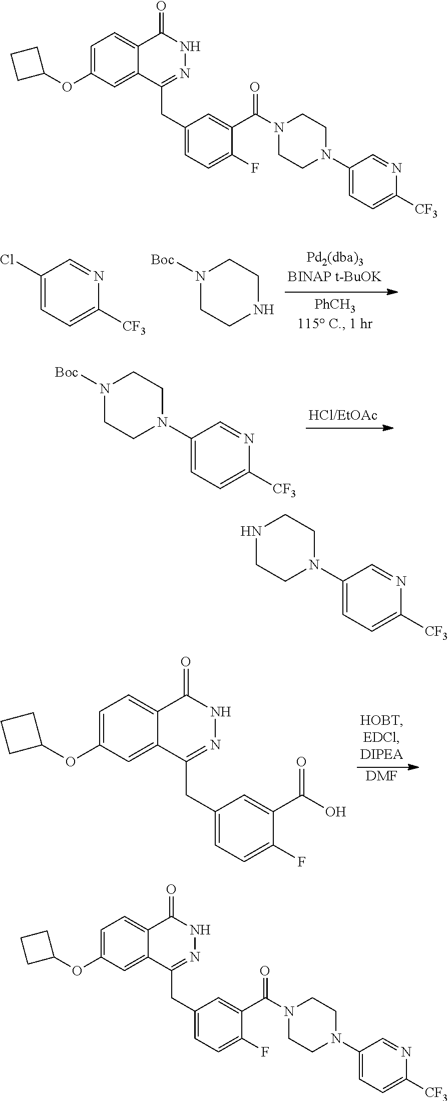

### Step 1, 2 and 3

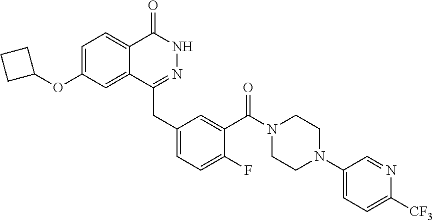 [SMILES: O=C(c1cc(Cc2n[nH]c(=O)c3ccc(OC4CCC4)cc23)ccc1F)N1CCN(c2ccc(C(F)(F)F)nc2)CC1]

### 6-cyclobutoxy-4-(4-fluoro-3-(4-(6-(trifluoromethyl)pyridin-3-yl)piperazine-1-carbonyl)benzyl)phthalazin-1(2H)-one

Following the general three-step procedure above in Example 130, but starting with 5-chloro-2-(trifluoromethyl)pyridine gave the title compound as a white solid. LCMS EST m/z: 581.7 [M+H]⁺. ¹H NMR (400 MHz, DMSO-D6) δ 12.45 (s, 1H), 8.43 (d, J=2.8 Hz, 1H), 8.17 (d, J=8.8 Hz, 1H), 7.68 (d, J=8.8 Hz, 1H), 7.48-7.39 (m, 3H), 7.35-7.23 (m, 2H), 7.12 (d, J=2.3 Hz, 1H), 4.89-4.79 (m, 1H), 4.32 (s, 2H), 3.79 (s, 2H), 3.47 (s, 2H), 3.34 (s, 2H), 3.31-3.26 (m, 2H), 2.39 (m, 2H), 2.00 (m, 2H), 1.72 (m, 2H).

### Synthesis of Example 160: 6-cyclobutoxy-4-(4-fluoro-3-(4-(5-(trifluoromethyl)pyrazin-2-yl)piperazine-1-carbonyl)benzyl)phthalazin-1(2H)-one

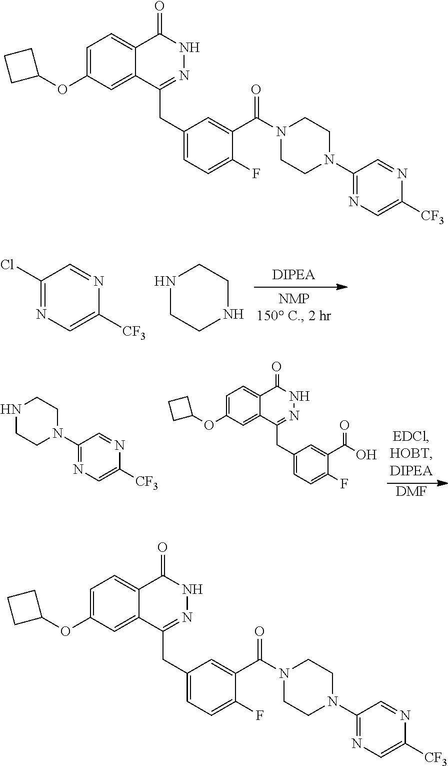

### Step 1,

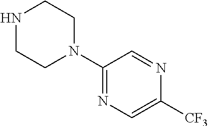 [SMILES: FC(F)(F)c1cnc(N2CCNCC2)cn1]

### 2-(Piperazin-1-yl)-5-(trifluoromethyl)pyrazine

To a solution of 2-chloro-5-(trifluoromethyl)pyrazine (100 mg, 547.85 μmol) in NMP (3 mL) was added tert-butyl piperazine-1-carboxylate (102.04 mg, 547.85 μmol). DIPEA (354.02 mg, 2.74 mmol, 477.12 μL), and then the sealed tube was irradiated microwave at 150° C. for 2 hr. The reaction mixture was diluted in EA (10 mL), washed with water (10 mL*2), brine, dried and concentrated. The residue was purified by silica gel chromatography to give 2-piperazin-1-yl-5-(trifluoromethyl)pyrazine (65 mg, 51.0% yield) as a grey solid. LCMS ESI m/z: 233.1 [M+H]⁺.

### Step 2,

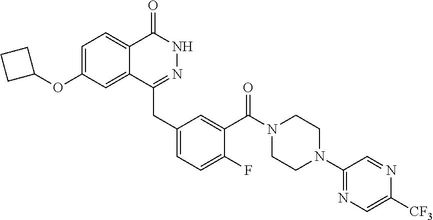 [SMILES: O=C(c1cc(Cc2n[nH]c(=O)c3ccc(OC4CCC4)cc23)ccc1F)N1CCN(c2cnc(C(F)(F)F)cn2)CC1]

### 6-cyclobutoxy-4-(4-fluoro-3-(4-(5-(trifluoromethyl)pyrazin-2-yl)piperazine-1-carbonyl)benzyl)phthalazin-1(2H)-one

Following the general one-step procedure above in Example 130, but starting with 2-(piperazin-1-yl)-5-(trifluoromethyl)pyrazine gave the title compound as a white solid. LCMS ESI m/z: 582.7 [M+H]⁺. ¹H NMR (400 MHz, DMSO-D6) δ 12.44 (s, 1H), 8.51 (s, 1H), 8.42 (s, 1H), 8.16 (d, J=8.8 Hz, 1H), 7.43 (dd, J=9.5, 6.0 Hz, 2H), 7.35-7.23 (m, 2H), 7.11 (d, J=2.3 Hz, 1H), 4.90-4.78 (m, 1H), 4.31 (s, 2H), 3.82 (d, J=5.3 Hz, 2H), 3.76 (s, 2H), 3.67 (s, 2H), 3.33 (s, 2H), 2.45-2.35 (m, 2H), 2.07-1.95 (m, 2H), 1.84-1.61 (m, 2H).

### Synthesis of Example 161: 6-cyclobutoxy-4-(3-(4-(5-(1,1-difluoroethyl)pyrimidin-2-yl)piperazine-1-carbonyl)-4-fluorobenzyl)phthalazin-1(2H)-one

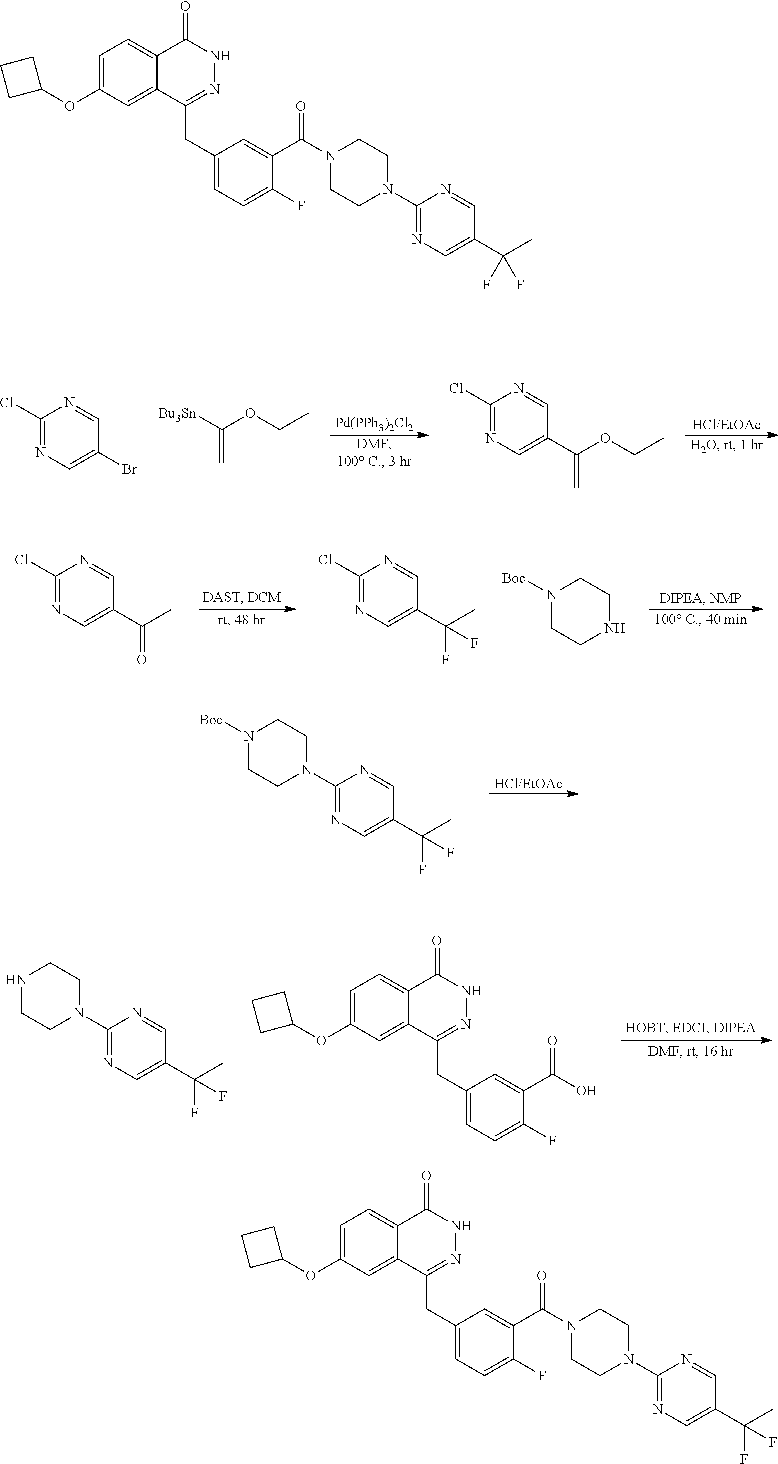

### Step 1

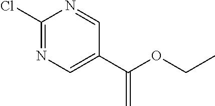

### 2-Chloro-5-(1-ethoxyvinyl)pyrimidine

To a solution of tributyl(1-ethoxyvinyl)stannane (3.73 g, 10.34 mmol, 3.49 mL) in DMF (15 mL) was added 5-bromo-2-chloro-pyrimidine (2.0 g, 10.34 mmol). Pd(PPh₃)₂Cl₂ (362.87 mg, 516.99 μmol). The reaction mixture was heated to 100° C. for 3 hr. Water (50 mL) and KF (1 g) was added to the reaction mixture, the mixture was stirred at rt for another 10 minutes, extracted with EtOAc (50 mL). The organic layer was washed with brine, dried and concentrated to give 2-chloro-5-(1-ethoxyvinyl)pyrimidine (2.2 g, crude) as a brown solid.

### Step 2,

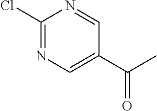

### 1-(2-Chloropyrimidin-5-yl)ethanone

To a mixture of 2-chloro-5-(1-ethoxyvinyl)pyrimidine (2.2 g, 11.92 mmol) in H₂O (40 mL) was added HC/EtOAc (4 M, 10 mL), and then stirred at rt for 1 hr. The mixture was extracted with EtOAc (40 mL), the organic layer was washed with brine, dried, and concentrated to the residue. The residue was purified by silica gel chromatography to give 1-(2-chloropyrimidin-5-yl)ethanone (900 mg, 48% yield) as a yellow solid. LCMS EST m/z: 157.1 [M+H]⁺.

### Step 3,

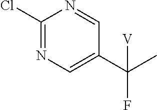

### 2-chloro-5-(1,1-difluoroethyl)pyrimidine

To a solution of 1-(2-chloropyrimidin-5-yl)ethanone (500 mg, 3.19 mmol) in DCM (10 mL) was added DAST (5.15 g, 31.93 mmol, 4.22 mL). The reaction mixture was stirred at rt for 48 hr. The mixture was cooled to 0° C., and sat. NaHCO₃ was added. The organic phase was partitioned, washed with brine, dried and concentrated to a residue. The residue was purified by silica gel chromatography to give 2-chloro-5-(1,1-difluoroethyl)pyrimidine (400 mg, 70.1% yield) as a white solid. ¹H NMR (400 MHz, CDCl₃) δ 8.77 (s, 2H), 2.00 (t, J=18.4 Hz, 3H).

### Step 4, 5 and 6

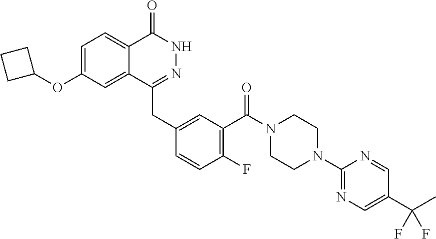 [SMILES: CC(F)(F)c1cnc(N2CCN(C(=O)c3cc(Cc4n[nH]c(=O)c5ccc(OC6CCC6)cc45)ccc3F)CC2)nc1]

### 6-Cyclobutoxy-4-(3-(4-(5-(1,1-difluoroethyl)pyrimidin-2-yl)piperazine-1-carbonyl)-4-fluorobenzyl)phthalazin-1(2H)-one

Following the general three-step procedure above in Example 130, but starting with 2-chloro-5-(1,1-difluoroethyl)pyrimidine gave the title compound as a white solid. LCMS EST m/z: 578.7 [M+H]⁺. ¹H NMR (400 MHz, DMSO-d₆) δ 12.45 (s, 1H), 8.59 (s, 2H), 8.16 (d, J=8.8 Hz, 1H), 7.42 (dd, J=6.9, 4.8 Hz, 2H), 7.34-7.22 (m, 2H), 7.10 (d, J=2.3 Hz, 1H), 4.89-4.78 (m, 1H), 4.31 (s, 2H), 3.88 (s, 2H), 3.73 (d, J=3.7 Hz, 4H), 3.30 (d, J=5.9 Hz, 2H), 2.39 (dd, J=9.0, 6.0 Hz, 2H), 1.99 (dd, J=24.3, 13.5 Hz, 5H), 1.72 (dd, J=48.2, 10.2 Hz, 2H).

### Synthesis of Example 162: 6-cyclobutoxy-4-(3-(4-(5-cyclopropylpyrimidin-2-yl)piperazine-1-carbonyl)-4-fluorobenzyl)phthalazin-1(2H)-one

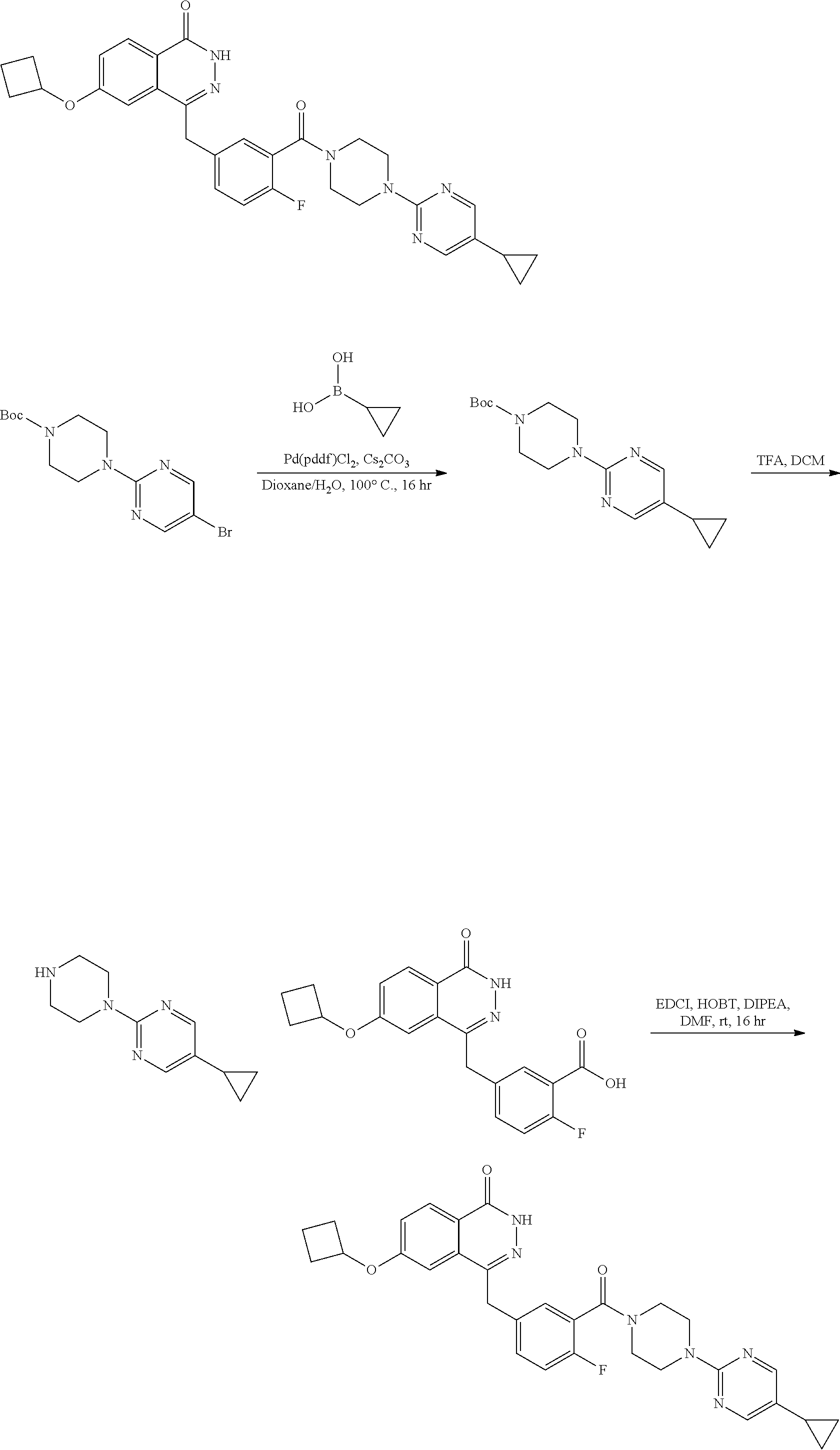

### Step 1

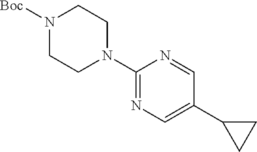 [SMILES: CC(C)(C)OC(=O)N1CCN(c2ncc(C3CC3)cn2)CC1]

### tert-Butyl 4-(5-cyclopropylpyrimidin-2-yl)piperazine-1-carboxylate

A mixture of tert-butyl 4-(5-bromopyrimidin-2-yl)piperazine-1-carboxylate (500 mg, 1.46 mmol), cyclopropylboronic acid (150.16 mg, 1.75 mmol), dicesium carbonate (949.30 mg, 2.91 mmol), cyclopentyl(diphenyl)phosphane dichloromethane dichloropalladium iron (1.43 g, 1.75 mmol) and water (0.5 mL) in Dioxane (2 mL) was stirred under N₂ at 100° C. for 16 hr. The reaction mixture was concentrated to a residue. The residue was purified by silica gel chromatography (Petroleum ether:EtOAc=10:1) to give tert-butyl 4-(5-cyclopropylpyrimidin-2-yl)piperazine-1-carboxylate (200 mg, 45% yield) as a white solid. LCMS ESI m/z: 305.2 [M+H]⁺.

### Step 2 and 3

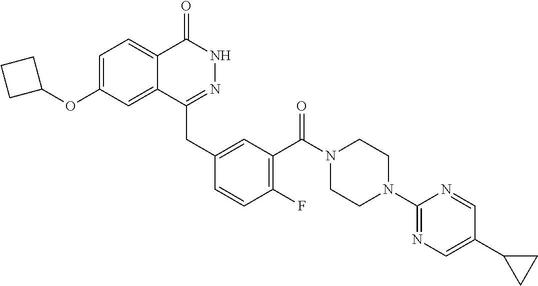 [SMILES: O=C(c1cc(Cc2n[nH]c(=O)c3ccc(OC4CCC4)cc23)ccc1F)N1CCN(c2ncc(C3CC3)cn2)CC1]

### 6-Cyclobutoxy-4-(3-(4-(5-cyclopropylpyrimidin-2-yl)piperazine-1-carbonyl)-4-fluorobenzyl)phthalazin-1(2H)-one

Following the general two-step procedure above in Example 130, but starting with tert-butyl 4-(5-cyclopropylpyrimidin-2-yl)piperazine-1-carboxylate gave the title compound as a white solid. LCMS ESI m/z: 555.3 [M+H]1. ¹H NMR (400 MHz, DMSO-d₆) δ 12.44 (s, 1H), 8.19 (s, 2H), 8.15 (d, J=8.8 Hz, 1H), 7.41 (dd, J=6.1, 2.0 Hz, 2H), 7.31 (dd, J=8.8, 2.1 Hz, 1H), 7.25 (t, J=9.3 Hz, 1H), 7.10 (d, J=2.1 Hz, 1H), 4.88-4.77 (m, 1H), 4.31 (s, 2H), 3.78-3.58 (m, 6H), 3.25 (s, 2H), 2.39 (m, 2f), 2.00 (m, 2H), 1.81-1.72 (m, 2H), 1.66 (dd, J=19.0, 9.1 Hz, 1H), 0.91-0.84 (m, 2H), 0.67-0.60 (m, 2H).

### Synthesis of Example 163: 6-Cyclobutoxy-4-(3-(3-(difluoromethyl)-5,6,7,8-tetrahydro-[1,2,4]triazolo[4,3-a]pyrazine-7-carbonyl)-4-fluorobenzyl)phthalazin-1(2H)-one

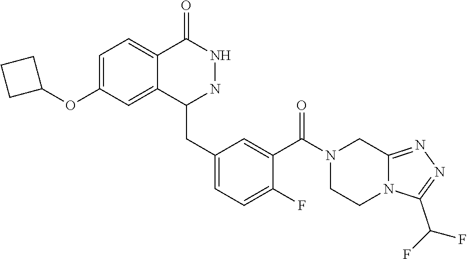 [SMILES: O=C1NNC(Cc2ccc(F)c(C(=O)N3CCn4c(nnc4C(F)F)C3)c2)c2cc(OC3CCC3)ccc21]

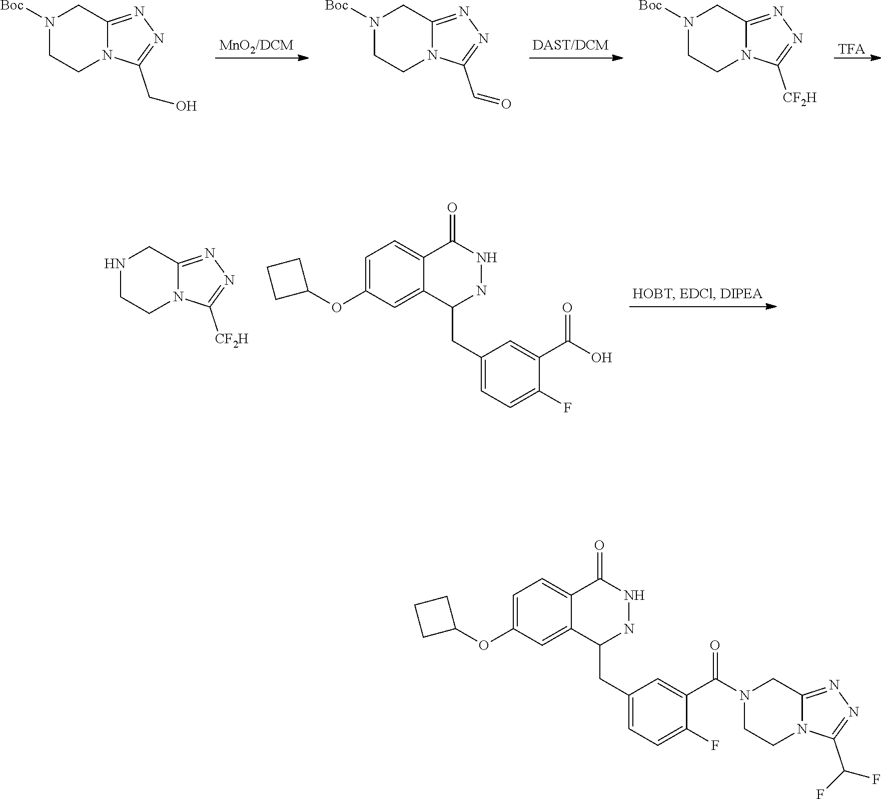

### Step 1

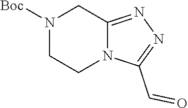 [SMILES: CC(C)(C)OC(=O)N1CCn2c(C=O)nnc2C1]

### tert-Butyl 3-formyl-5,6-dihydro-[1,2,4]triazolo[4,3-a]pyrazine-7(8H)-carboxylate

A solution of tert-butyl 3-(hydroxymethyl)-5,6-dihydro-[1,2,4]triazolo[4,3-a]pyrazine-7(8H)-carboxylate (0.2 g, 1.30 mmol) and MnO₂ (1.13 g, 12.97 mmol) in DCM (5 mL) was stirred for 16 hr at 25° C. The reaction mixture was filtered through a pad of celite, and the filtrate was concentrated to give tert-butyl 3-formyl-5,6-dihydro-[1,2,4]triazolo[4,3-a]pyrazine-7(8H)carboxylate (0.2 g, 91% yield). LCMS ESI m/z: 252.9 [M+H]⁺. ¹H NMR (400 MHz, DMSO-d₆) δ 9.97 (s, 1H), 4.80 (br s, 2H), 4.04-4.02 (m, 2H), 3.78-3.74 (m, 2H), 1.44 (s, 9H).

### Step 2, 3 and 4

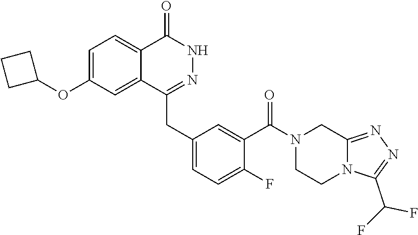 [SMILES: O=C(c1cc(Cc2n[nH]c(=O)c3ccc(OC4CCC4)cc23)ccc1F)N1CCn2c(nnc2C(F)F)C1]

### 6-Cyclobutoxy-4-(3-(3-(difluoromethyl)-5,6,7,8-tetrahydro-[1,2,4]triazolo[4,3-a]pyrazine-7-carbonyl)-4-fluorobenzyl)phthalazin-1(2H)-one

Following the general three-step procedure above in Example 130, but starting with tert-butyl 3-formyl-5,6-dihydro-[1,2,4]triazolo[4,3-a]pyrazine-7(8H)-carboxylate gave the title compound as a white solid. LCMS ESI m/z: 525.2 [M+H]⁺. ¹H NMR (400 MHz, DMSO-d₆) δ 12.43 (s, 1H), 8.15 (d, J=8.8 Hz, 1H), 7.54-7.43 (m, 2H), 7.40-7.15 (m, 3H), 7.07 (s, 1H), 5.00 (s, 1H), 4.89-4.79 (m, 1H), 4.66 (s, 1H), 4.31 (s, 2H), 4.25-3.94 (m, 3H), 3.69 (s, 1H), 2.37 (s, 2H), 0.99 (m, 2H), 1.84-1.70 (m, 1H), 1.69-1.58 (m, 1H).

### Synthesis of Example 164: 6-Cyclobutoxy-4-(4-fluoro-3-(3-(fluoromethyl)-5,6,7,8-tetrahydro-[1,2,4]triazolo[4,3-a]pyrazine-7-carbonyl)benzyl)phthalazin-1(2H)-one

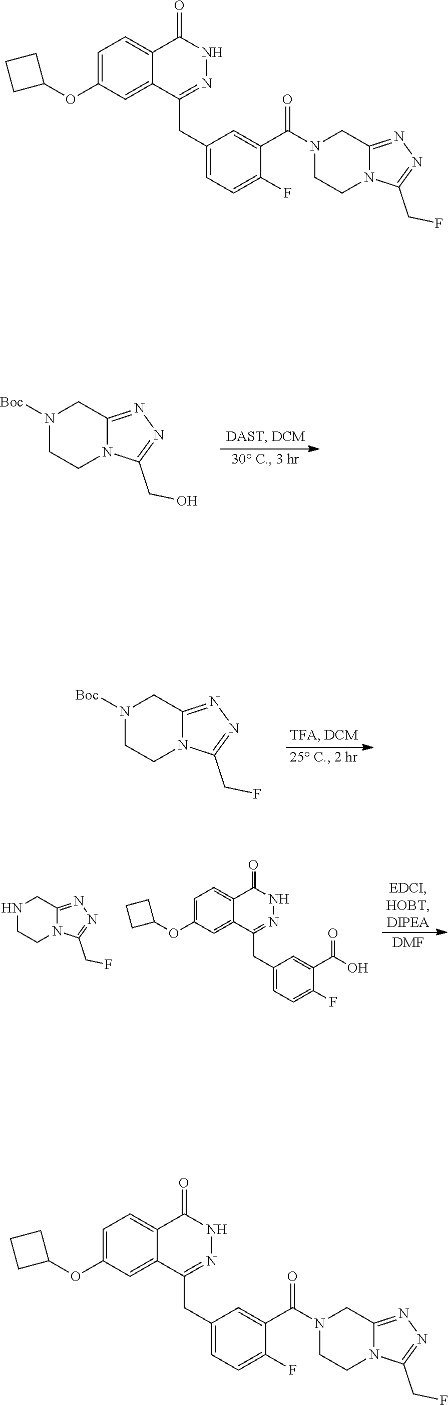

### Step 1

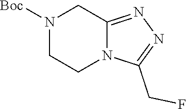 [SMILES: CC(C)(C)OC(=O)N1CCn2c(CF)nnc2C1]

### tert-Butyl 3-(fluoromethyl)-5,6-dihydro-[1,2,4]triazolo[4,3-a]pyrazine-7(8H)-carboxylate

To a solution of tert-butyl 3-(hydroxymethyl)-5,6-dihydro-[1.2.4]triazolo[4,3-a]pyrazine-7(8H)-carboxylate (100 mg, 393.26 μmol) in dry DCM (4.0 mL) was added dropwise DAST (633.90 mg, 3.93 mmol, 519.59 μL). The reaction mixture was stirred at 30° C. for 2 hr. The mixture was poured into sat. NaHCO₃, extracted with DCM (10 mL×3). The extracts were washed with brine, dried over Na₂SO₄, filtered and concentrated. The crude was purified by silica gel chromatography to give tert-butyl 3-(fluoromethyl)-5,6-dihydro-[1,2,4]triazolo[4,3-a]pyrazine-7(8H)-carboxylate (70 mg, 65.9% yield) as a yellow solid. ¹H NMR (400 MHz, DMSO-di) a 5.64 (d, J=48 Hz, 1H), 4.71 (br, 2H), 4.09-4.06 (m, 2H), 3.81-3.78 (m, 2H), 1.49 (s, 9H).

### Step 2 and 3

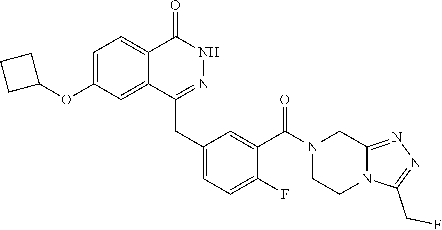 [SMILES: O=C(c1cc(Cc2n[nH]c(=O)c3ccc(OC4CCC4)cc23)ccc1F)N1CCn2c(CF)nnc2C1]

### 6-Cyclobutoxy-4-(4-fluoro-3-(3-(fluoromethyl)-5,6,7,8-tetrahydro-[1,2,4]triazolo[4,3-a]pyrazine-7-carbonyl)benzyl)phthalazin-1(2H)-one

Following the general two-step procedure above in Example 130, but starting with tert-butyl 3-(fluoromethyl)-5,6-dihydro-[1,2,4]triazolo[4,3-a]pyrazine-7(8H)-carboxylate gave the title compound as a white solid. LCMS EST m/z: 506.7 [M+H]⁺. ¹H NMR (400 MHz, DMSO-d₆) δ 12.43 (s, 1H), 8.16 (d, J=8.7 Hz, 1H), 7.52-7.42 (m, 2H), 7.34-7.26 (m, 2H), 7.08 (s, 1H), 5.59 (m, 2H), 5.00-4.59 (m, 3H), 4.32 (s, 2H), 4.21-3.86 (m, 3H), 3.67 (s, 1H), 2.37 (s, 2H), 2.06-1.93 (m, 2H) 1.70 (m, 2H).

### Synthesis of Example 165: 6-Cyclobutoxy-4-(4-fluoro-3-(3-methoxy-5,6,7,8-tetrahydro-[1,2,4]triazolo[4,3-a]pyrazine-7-carbonyl)benzyl)phthalazin-1(2H)-one

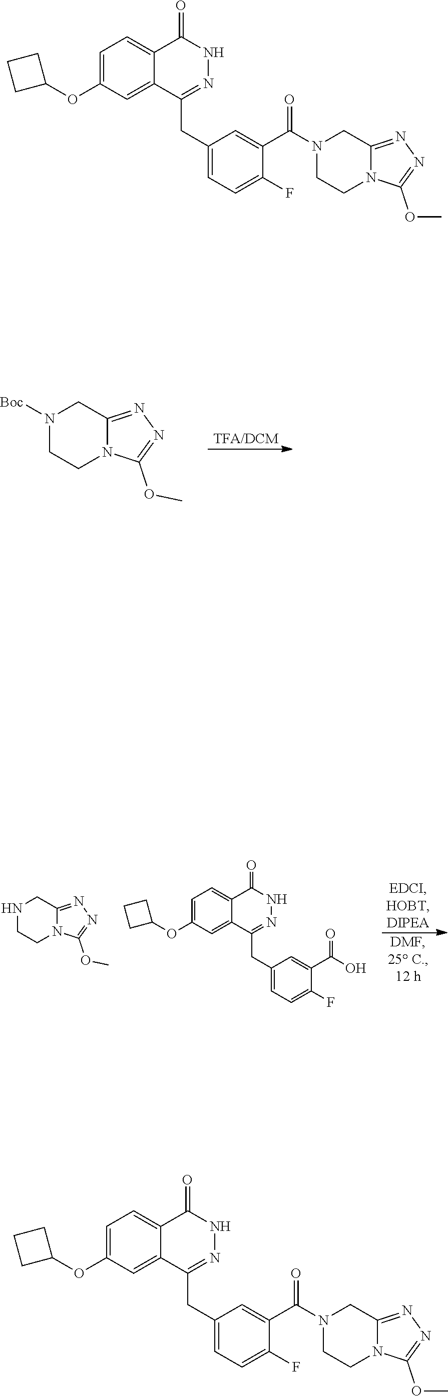

### Step 1

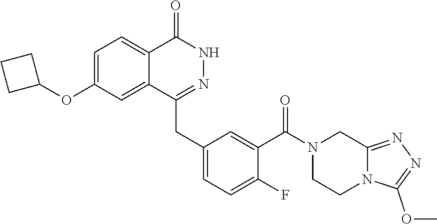 [SMILES: COc1nnc2n1CCN(C(=O)c1cc(Cc3n[nH]c(=O)c4ccc(OC5CCC5)cc34)ccc1F)C2]

### 6-Cyclobutoxy-4-(4-fluor-3-(3-methoxy-5,6,7,8-tetrahydro-[1,2,4]triazolo[4,3-a]pyrazine-7-carbonyl)benzyl)phthalazin-1(2H)-one

Following the general two-step procedure above in Example 130, but starting with tert-butyl 3-methoxy-5,6-dihydro-[1.2.4]triazolo[4,3-a]pyrazine-7(8H)-carboxylate gave the title compound as a white solid. LCMS ESI m/z: 504.8 [M+H]⁺. ¹H NMR (400 MHz, DMSO-D6) δ 12.43 (s, 1H), 8.16 (d, J=8.8 Hz, 1H), 7.46 (d, J=5.9 Hz, 2H), 7.34-7.25 (m, 2H), 7.08 (d, J=2.2 Hz, 1H), 4.83 (m, 2H), 4.46 (s, 1H), 4.30 (s, 2H), 4.03 (m, 4H), 3.79 (s, 1H), 3.61 (s, 2H), 2.43-2.33 (m, 2H), 1.99 (m, 2H), 1.83-1.61 (m, 2H).

### Synthesis of Example 166: 6-Cyclobutoxy-4-(4-fluoro-3-(3-(hydroxymethyl)-5,6,7,8-tetrahydro-[1,2,4]triazolo[4,3-a]pyrazine-7-carbonyl)benzyl)phthalazin-1(2H)-one

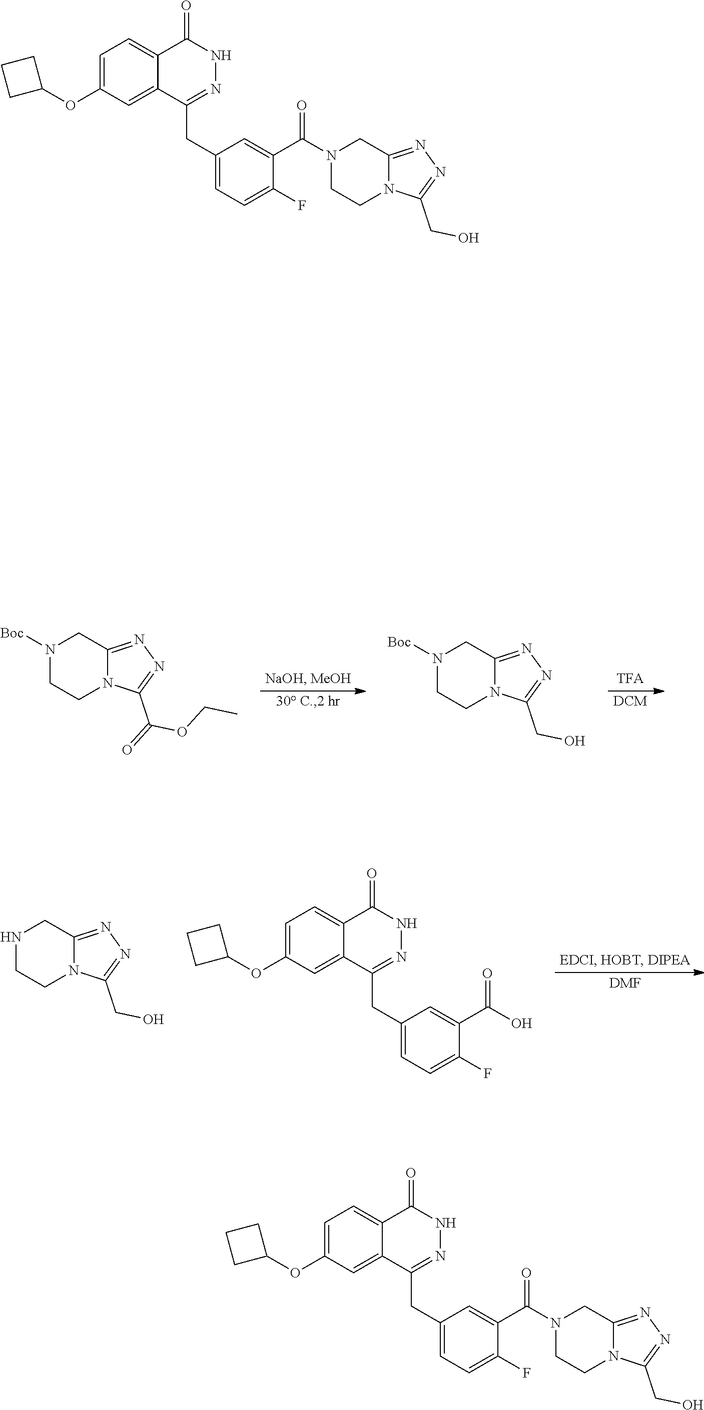

### Step 1,

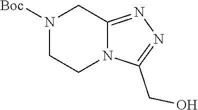 [SMILES: CC(C)(C)OC(=O)N1CCn2c(CO)nnc2C1]

### tert-butyl 3-(hydroxymethyl)-5,6-dihydro-[1,2,4]triazolo[4,3-a]pyrazine-7(8H)-carboxylate

To a solution of 7-tert-butyl 3-ethyl 5,6-dihydro-[1.2.4]triazolo[4,3-a]pyrazine-3,7(8H)-dicarboxylate (2.0 g, 6.75 mmol) in MeOH (60 mL) was added NaBH₄ (2.05 g, 54.00 mmol). The mixture was stirred at 30° C. for 2 hr. Water (50 mL) was added and stirred for another 10 min. The reaction mixture was concentrated to give a crude solid. The crude product was purified by silica gel chromatography (DCM:MeOH=10:1) to give tert-butyl 3-(hydroxymethyl)-5,6-dihydro-[1,2,4]triazolo[4,3-a]pyrazine-7(8H)-carboxylate (1.2 g, 66% yield) as a white solid. LCMS ESI m/z: 255.2 [M+H]⁺.

### Step 2 and 3

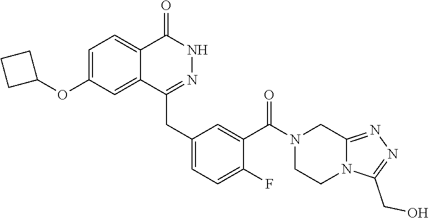 [SMILES: O=C(c1cc(Cc2n[nH]c(=O)c3ccc(OC4CCC4)cc23)ccc1F)N1CCn2c(CO)nnc2C1]

### 6-Cyclobutoxy-4-(4-fluoro-3-(3-(hydroxymethyl)-5,6,7,8-tetrahydro-[1,2,4]triazolo[4,3-a]pyrazine-7-carbonyl)benzyl)phthalazin-1(2H)-one

Following the general two-step procedure above in Example 130, but starting with tert-butyl 3-(hydroxymethyl)-5,6-dihydro-[1,2,4]triazolo[4,3-a]pyrazine-7(8H)-carboxylate gave the title compound as a white solid. LCMS ESI m/z: 504.7 [M+H]⁺. ¹H NMR (400 MHz, DMSO-D6) δ 12.43 (s, 1H), 8.16 (dd, J=8.7, 4.9 Hz, 1H), 7.50-7.43 (m, 2H), 7.30 (dd, J=12.6, 5.1 Hz, 2H), 7.08 (s, 1H), 5.49 (dd, J=12.5, 6.9 Hz, 1H), 4.92 (s, 1H), 4.83 (p, J=7.1 Hz, 1H), 4.69-4.46 (m, 3H), 4.32 (s, 2H), 4.19-3.84 (m, 3H), 3.65 (s, 1H), 2.38 (s, 2H), 2.06-1.94 (m, 2H), 1.84-1.73 (m, 1H), 1.71-1.59 (m, 1H).

### Synthesis of Example 167: 6-Cyclobutoxy-4-(4-fluoro-3-(3-(methoxymethyl)-5,6,7,8-tetrahydro-[1,2,4]triazolo[4,3-a]pyrazine-7-carbonyl)benzyl)phthalazin-1(2H)-one

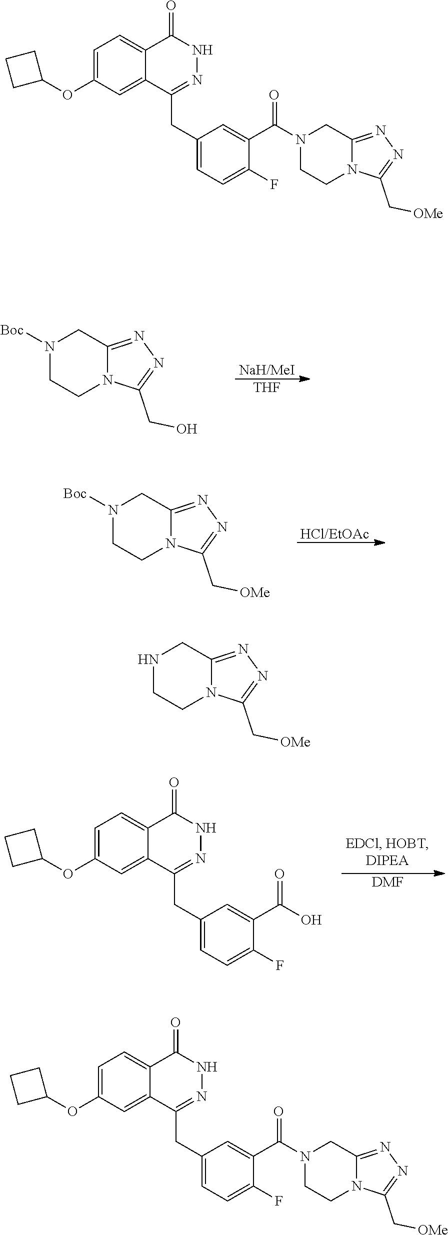

### Step 1,

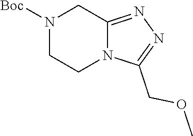 [SMILES: COCc1nnc2n1CCN(C(=O)OC(C)(C)C)C2]

### tert-Butyl 3-(methoxymethyl)-5,6-dihydro-[1,2,4]triazolo[4,3-a]pyrazine-7(8H)-carboxylate

A mixture of tert-butyl 3-(hydroxymethyl)-6,8-dihydro-5H-[1.2.4]triazolo[4,3-a]pyrazine-7-carboxylate (150 mg, 589.89 μmol) and NaH (54.25 mg, 2.36 mmol) in dry THF (10 mL) was stirred at 0° C. for 15 min. Then CH₃I (0.47 g, 3.3 mmol) was added dropwise to the reaction mixture. The reaction mixture was allowed to warm to room temperature and stirred for another 2 hr. Sat. NH₄Cl solution was added and the resulting mixture was extracted with ethyl acetate. The combined organic layers were washed with brine, dried over anhydrous Na₂SO₄ and concentrated to give tert-butyl 3-(methoxymethyl)-5,6-dihydro-[1,2,4]triazolo[4,3-a]pyrazine-7(8H)-carboxylate (120 mg, 76% yield) as a white solid. The residue was used for next step directly. LCMS ESI m/z: 269.2 [M+H]⁺.

### Step 2 and 3

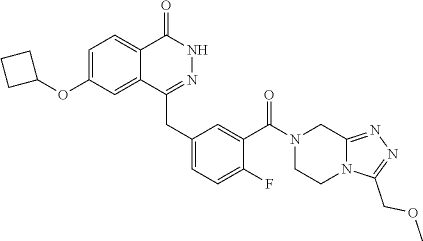 [SMILES: COCc1nnc2n1CCN(C(=O)c1cc(Cc3n[nH]c(=O)c4ccc(OC5CCC5)cc34)ccc1F)C2]

### 6-Cyclobutoxy-4-(4-fluoro-3-(3-(methoxymethyl)-5,6,7,8-tetrahydro-[1,2,4]triazolo[4,3-a]pyrazine-7-carbonyl)benzyl)phthalazin-1(2H)-one

Following the general two-step procedure above in Example 130, but starting with tert-butyl 3-(methoxymethyl)-5,6-dihydro-[1.2.4]triazolo[4,3-a]pyrazine-7(8H)-carboxylate gave the title compound as a white solid. LCMS ESI m/z: 519.2 [M+H]⁺. ¹H NMR (400 MHz, DMSO-d4) δ 12.45 (s, 1H), 8.17 (t. J=7.3 Hz, 1H), 7.46 (d, J=6.6 Hz, 2H), 7.36-7.26 (m, 2H), 7.09 (d, J=6.2 Hz, 1H), 4.94 (s, 1H), 4.89-4.81 (m, 1H), 4.65-4.48 (m, 3H), 4.33 (s, 2H), 4.09 (s, 2H), 3.87 (s, 1H), 3.65 (s, 1H), 3.29 (d, J=2.8 Hz, 3H), 2.38 (s, 2H), 2.00 (s, 2H), 1.83-1.75 (m, 1H), 1.67 (m, 1H).

### Synthesis of Example 168: 6-Cyclobutoxy-4-(4-fluoro-3-(2-(hydroxymethyl)-4,5,6,7-tetrahydropyrazolo[1,5-a]pyrazine-5-carbonyl)benzyl)phthalazin-1(2H)-one

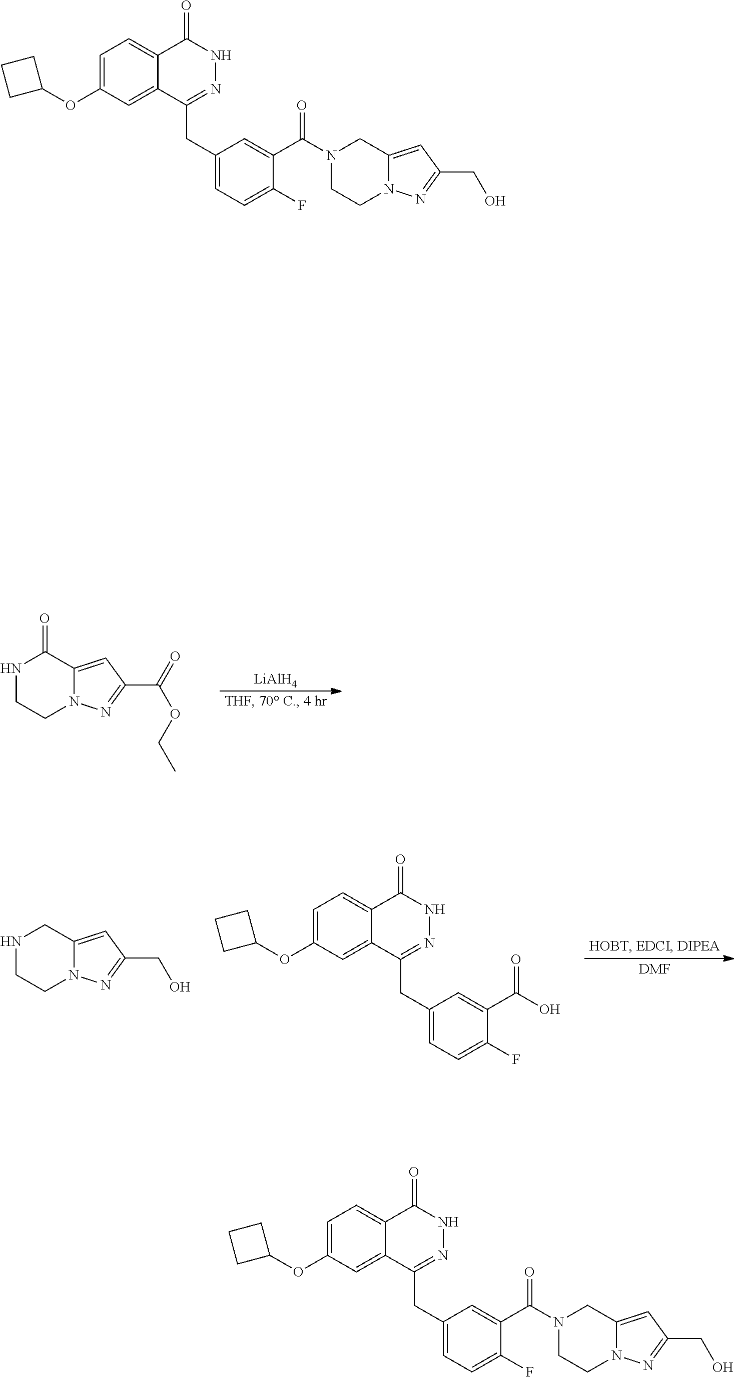

### Step 1

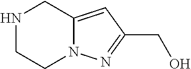 [SMILES: OCc1cc2n(n1)CCNC2]

### (4,5,6,7-Tetrahydropyrazolo[1,5-a]pyrazin-2-yl)methanol

To a solution of ethyl 4-oxo-6,7-dihydro-5H-pyrazolo[1,5-a]pyrazine-2-carboxylate (200 mg, 956.02 μmol) in THF (10 mL) was added LiAlH₄ (145.14 mg, 3.82 mmol) at 0° C. and then the mixture was heated to 70° C. for 4 hr. The reaction solution was sequentially quenched with H₂O (200 mg), 15% NaOH (40) mg) and H₂O (200 mg). The mixture was stirred at rt for 10 minutes. The solids were removed by filtration, and the filtrate was concentrated to give (4,5,6,7-tetrahydropyrazolo[1,5-a]pyrazin-2-yl)methanol (110 mg, crude) as a light yellow oil, and it will be used in the next step directly. LCMS EST m/z: 154.0 [M+H]⁺.

### Step 2

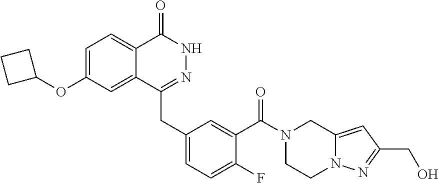 [SMILES: O=C(c1cc(Cc2n[nH]c(=O)c3ccc(OC4CCC4)cc23)ccc1F)N1CCn2nc(CO)cc2C1]

### 6-Cyclobutoxy-4-(4-fluoro-3-(2-(hydroxymethyl)-4,5,6,7-tetrahydropyrazolo[1,5-a]pyrazine-5-carbonyl)benzyl)phthalazin-1(2H)-one

Following the general one-step procedure above in Example 130, but starting with (4,5,6,7-tetrahydropyrazolo[1,5-a]pyrazin-2-yl)methanol gave the title compound as a white solid. LCMS ESI m/z: 503.8 [M+H]⁺. ¹H NMR (400 MHz, DMSO-d₆) δ 12.43 (s, 1H), 8.15 (dd, J=8.7, 3.9 Hz, 1H), 7.45 (dd, J=7.0, 3.8 Hz, 2H), 7.29 (td. J=8.9, 4.7 Hz, 2H), 7.14-7.05 (m, 1H), 6.02 (m, 1H), 5.01-4.91 (m, 1H), 4.82 (m, 2H), 4.50-4.27 (m, 4H), 4.10 (s, 2H), 3.97 (d, J=5.2 Hz, 1H), 3.66 (s, 1H), 2.36 (m, 2H), 1.99 (m, 2H), 1.86-1.56 (m, 2H).

### Synthesis of Example 169: 4-(4-chloro-3-(4-(5-(trifluromethyl)pyrimidin-2-yl)piperazine-1-carbonyl)benzyl)-6-cyclobutoxyphthalazin-1(2H)-one

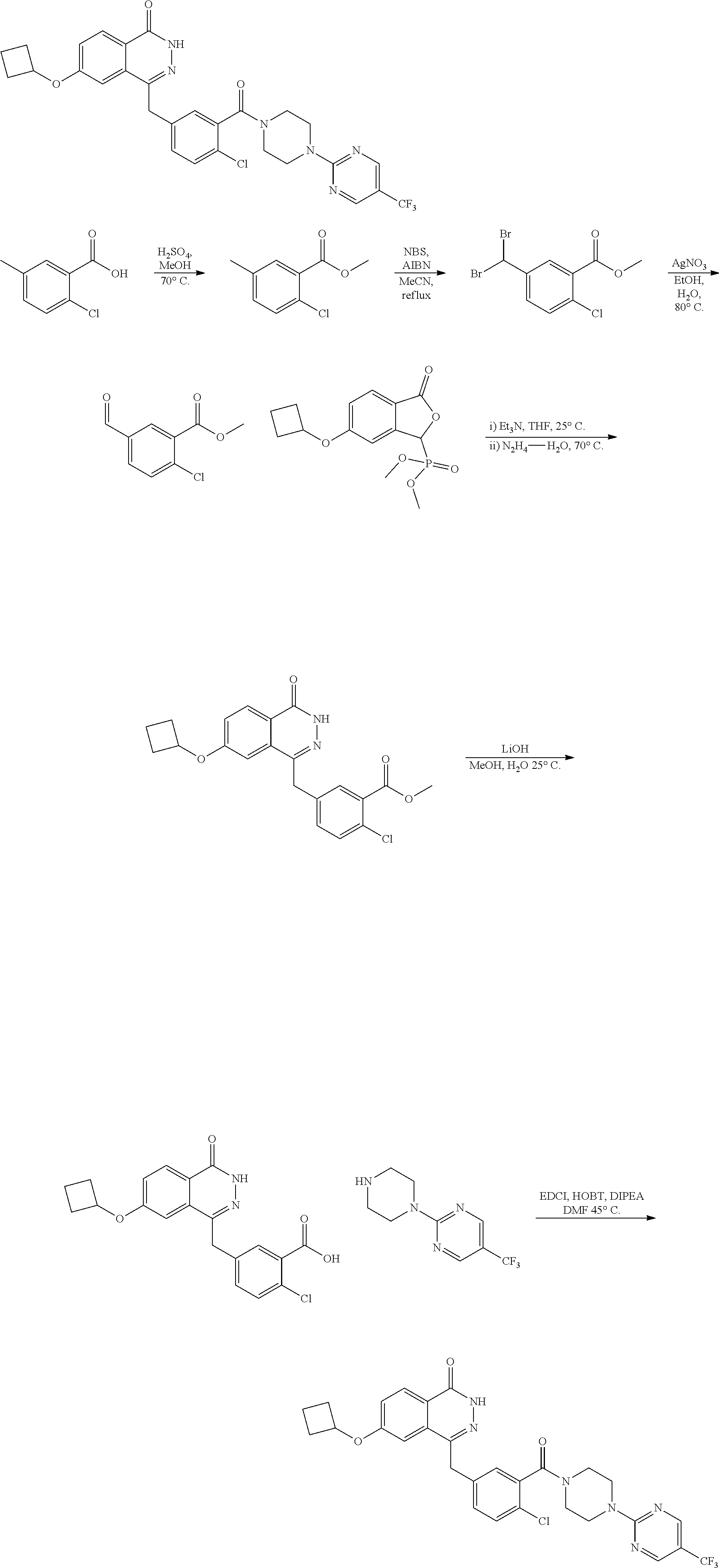

### Step 1

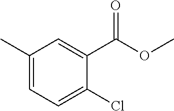

### Methyl 2-chloro-5-methylbenzoate

To a solution of 2-chloro-5-methylbenzoic acid (5.0) g, 29.31 mmol) in MeOH (20 mL) was added conc. H₂SO₄ (5 mL) in portions. The mixture was stirred at 70° C. for 2 hours. The solution was concentrated in vacuo, the residue was diluted with water (30 mL) and extracted with EtOAc (20 mL×3). The combined organic layer was washed with brine, dried over anhydrous Na₂SO₄, filtered and concentrated in vacuo to give methyl 2-chloro-5-methylbenzoate (5.0 g, 91% yield) as a white oil. LCMS ESI m/z: 185.0 [M+H]⁺.

### Step 2

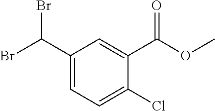

### Methyl 2-chloro-5-(dibromomethyl)benzoate

A mixture of methyl methyl 2-chloro-5-methylbenzoate (500 mg, 2.71 mmol). NBS (1.59 g, 8.94 mmol) and AIBN (133 mg, 0.81 mmol) in ACN (10 mL) was stirred under reflux for 16 hours. The mixture was cooled to rt, diluted with H₂O (30 mL), and extracted with DCM (30 mL×3). The combined organic layer was dried over anhydrous Na₂SO₄, filtered and concentrated in vacuo. The residue was purified by silica gel chromatography (EtOAc/petroleum ether=0% to 15%) to obtain methyl methyl 2-chloro-5-(dibromomethyl)benzoate (615 mg, 62% yield) as a yellow oil. LCMS ESI m/z: 341 [M+H]⁺.

### Step 3

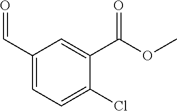

### Methyl 2-chloro-5-formylbenzoate

To a solution of methyl methyl 2-chloro-5-(dibromomethyl)benzoate (615 mg, 1.80 mmol) in EtOH (10 mL), was added AgNO₃ (3.05 g, 17.96 mmol) and H₂O (20 mL). The mixture was stirred at 80° C. for 30 minutes. The solution was concentrated in vacuo. This residue was diluted with water (30 mL) and extracted with EtOAc (20 mL×3). The combined organic layer was washed with brine, dried over anhydrous Na₂SO₄, filtered and concentrated in vacuo to give methyl 2-chloro-5-formylbenzoate (370 mg, 88% yield) as a yellow solid. LCMS ESI m/z: 199.0 [M+H]⁺.

### Step 4

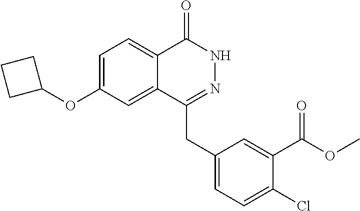 [SMILES: COC(=O)c1cc(Cc2n[nH]c(=O)c3ccc(OC4CCC4)cc23)ccc1Cl]

### methyl 2-chloro-5-((7-cyclobutoxy-4-oxo-3,4-dihydrophthalazin-1-yl)methyl) benzoate

A solution of dimethyl (6-cyclobutoxy-3-oxo-1,3-dihydroisobenzofuran-1-yl)phosphonate (200 mg, 0.64 mmol), methyl 2-chloro-5-formylbenzoate (134 mg, 0.67 mmol) and Et₃N (194 mg, 1.92 mmol) in anhydrous THF (6 mL) was stirred at 25° C. under Ar (g) for 3 hours. Hydrazine hydrate (49 mg, 0.83 mmol) was added and the reaction was stirred at 70° C. for 2 hours. The reaction was cooled to rt, then THF was removed under vacuum. The formed solids were washed with water (10 mL) and petroleum ether/EtOAc (10:1, 10 mL) to give methyl 2-chloro-5-((7-cyclobutoxy-4-oxo-3,4-dihydrophthalazin-1-yl)methyl)benzoate (125 mg, 49% yield) as a white solid. LCMS ESI m/z: 399.1 [M+H]⁺.

### Step 5

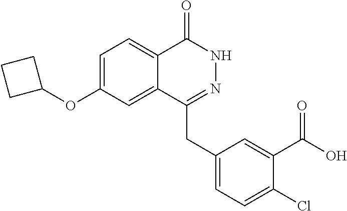 [SMILES: O=C(O)c1cc(Cc2n[nH]c(=O)c3ccc(OC4CCC4)cc23)ccc1Cl]

### 2-chloro-5-((7-cyclobutoxy-4-oxo-3,4-dihydrophthalazin-1-yl)methyl)benzoic acid

A solution of methyl 2-chloro-5-((7-cyclobutoxy-4-oxo-3,4-dihydrophthalazin-1-yl)methyl)benzoate (125 mg, 0.31 mmol) and LiOH (75 mg, 3.13 mmol) in MeOH (3 mL) and H₂O (1 mL) was stirred at rt for 30 minutes. The organic solvent was removed in vacuum and aq HCl (1N) was added until the solution reached to pH 5˜6. The solids were collected by filtration, washed with water and dried under vacuum to give 2-chloro-5-((7-cyclobutoxy-4-oxo-3,4-dihydrophthalazin-1-yl)methyl)benzoic acid (120 mg, 99% yield) as a white solid. LCMS ESI m/z: 385.1 [M+H]⁺.

### Step 6

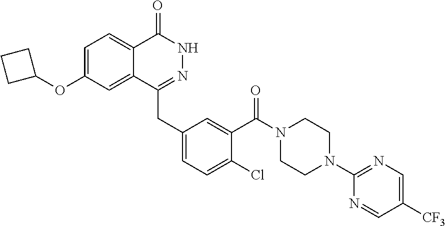 [SMILES: O=C(c1cc(Cc2n[nH]c(=O)c3ccc(OC4CCC4)cc23)ccc1Cl)N1CCN(c2ncc(C(F)(F)F)cn2)CC1]

### 4-(4-chloro-3-(4-(5-(trifluoromethyl)pyrimidin-2-yl)piperazine-1-carbonyl)benzyl)-6-cyclobutoxyphthalazin-1(2H)-one

To a solution of 2-(piperazin-1-yl)-5-(trifluoromethyl)pyrimidine (92 mg, 0.34 mmol) and 2-chloro-5-((7-cyclobutoxy-4-oxo-3,4-dihydrophthalazin-1-yl)methyl)benzoic acid (120 mg, 0.31 mmol) in DMF (3 mL) was added EDCI (90 mg, 0.47 mmol), HOBt (63 mg, 0.47 mmol) and DIPEA (201 mg, 1.56 mmol). The reaction mixture was stirred at 45° C. for 1 hour. The reaction was purified by prep-HPLC to give 4-(4-chloro-3-(4-(5-(trifluoromethyl)pyrimidin-2-yl)piperazine-1-carbonyl)benzyl)-6-cyclobutoxyphthalazin-1(2H)-one (98 mg, 52% yield) as a white solid. LCMS ESI m/z: 599.1 [M+H]⁺. ¹H NMR (400 MHz, CDCl₃) δ 10.54 (s, 1H), 8.49 (s, 2H), 8.36 (d, J=8.8 Hz, 1H), 7.37 (d, J=8.1 Hz, 1H), 7.28 (d, J=2.1 Hz, 1H), 7.25 (s, 1H), 7.19 (dd, J=8.8, 2.3 Hz, 1H), 6.89 (d, J=2.3 Hz, 1H), 4.66 (p, J=7.1 Hz, 1H), 4.23 (s, 2H), 4.09-3.77 (m, 6H), 3.39-3.19 (m, 2H), 2.48-2.36 (m, 2H), 2.22-2.09 (m, 2H), 1.94-1.70 (m, 2H).

### Synthesis of Example 170: 6-cyclobutoxy-4-(3-(4-(5-(trifluoromethyl)pyrimidin-2-yl)piperazine-1-carbonyl)benzyl)phthalazin-1(2H)-one

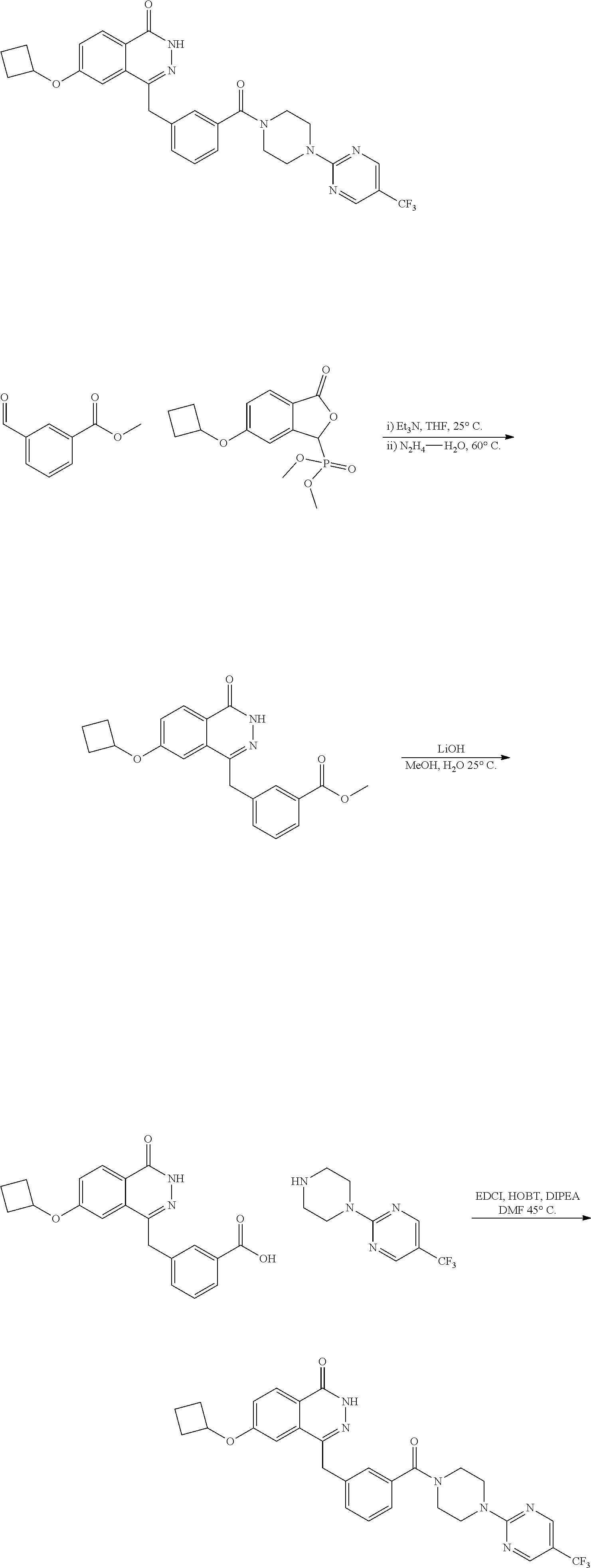

### Step 1

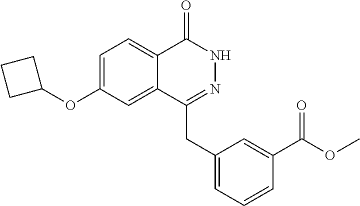 [SMILES: COC(=O)c1cccc(Cc2n[nH]c(=O)c3ccc(OC4CCC4)cc23)c1]

### Methyl 3-((7-cyclobutoxy-4-oxo-3,4-dihydrophthalazin-1-yl)methyl)benzoate

A solution of dimethyl (6-bromo-3-oxo-1,3-dihydroisobenzofuran-1-yl)phosphonate (200 mg, 0.64 mmol), methyl 3-formylbenzoate (105 mg, 0.64 mmol) and Et₃N (194 mg, 1.92 mmol) in anhydrous THF (5 mL) was stirred at 25° C. under Ar (g) for 15 hours. Hydrazine hydrate (43 mg, 0.86 mmol) was added and the reaction was stirred at 70° C. for 1 hour. The reaction was cooled to rt, then THF was removed under vacuum. The formed solid was washed with water (10 mL) and petroleum ether/EA (2:1, 10 mL) to give methyl 3-((7-cyclobutoxy-4-oxo-3,4-dihydrophthalazin-1-yl)methyl)benzoate (100 mg, 48% yield) as a white solid. LCMS ESI m/z: 365 [M+H]⁺.

### Step 2

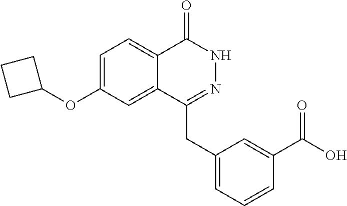 [SMILES: O=C(O)c1cccc(Cc2n[nH]c(=O)c3ccc(OC4CCC4)cc23)c1]

### 3-((7-Cyclobutoxy-4-oxo-3,4-dihydrophthalazin-1-yl)methyl)benzoic acid

A solution of methyl 3-((7-cyclobutoxy-4-oxo-3,4-dihydrophthalazin-1-yl)methyl)benzoate (100 mg, 0.27 mmol) and LiOH (66 mg, 2.70 mmol) in MeOH (3 mL) and H₂O (1 mL) was stirred at rt for 2 hours. The organic solvent was removed in vacuo and aq HCl (1 mol/L) was added until the solution reached pH 5˜6. The solids were collected by filtration, washed with water and dried under vacuum to give 3-((7-cyclobutoxy-4-oxo-3,4-dihydrophthalazin-1-yl)methyl)benzoic acid (80 mg, 83% yield) as a white solid. LCMS EST m/z: 351 [M+H]⁺.

### Step 3

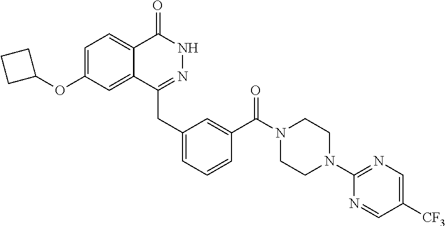 [SMILES: O=C(c1cccc(Cc2n[nH]c(=O)c3ccc(OC4CCC4)cc23)c1)N1CCN(c2ncc(C(F)(F)F)cn2)CC1]

### 6-Cyclobutoxy-4-(3-(4-(5-(trifluoromethyl)pyrimidin-2-yl)piperazine-1-carbonyl)benzyl)phthalazin-1(2H)-one

To a solution of 3-((7-cyclobutoxy-4-oxo-3,4-dihydrophthalazin-1-yl)methyl)benzoic acid (60 mg, 0.17 mmol) and 2-piperazin-1-yl)-5-(trifluoromethyl)pyrimidine (55 mg, 0.21 mmol) in DMF (2 mL) was added EDCI (49 mg, 0.26 mmol), HOBt (35 mg, 0.26 mmol) and DIPEA (111 mg, 0.86 mmol). The reaction mixture was stirred at 45° C. for 1 hour. The reaction was purified by prep-HPLC to give 6-cyclobutoxy-4-(3-(4-(5-(trifluoromethyl)pyrimidin-2-yl)piperazine-1-carbonyl)benzyl)phthalazin-1(2H)-one (34 mg, 35% yield) as a white solid (34 mg). LCMS ESI m/z: 565 [M+H]⁺. ¹H NMR (400 MHz, DMSO-d₆) δ 12.48 (s, 1H), 8.74 (s, 2H), 8.16 (d, J=8.8 Hz, 1H), 7.44-7.40 (m, 3H), 7.33-7.29 (m, 2H), 7.12-7.08 (m, 1H), 4.86-4.78 (m, 1H), 4.34 (s, 2H), 3.98-3.65 (m, 6H), 3.46-3.38 (m, 2H), 2.42-2.32 (m, 2H), 2.05-1.93 (m, 2H), 1.81-1.72 (m, 1H), 1.69-1.60 (m, 1H).

### Synthesis of Example 171: 4-(2-chloro-3-(4-(5-(trifluromethyl)pyrimidin-2-yl)piperazine-1-carbonyl)benzyl)-6-cyclobutoxyphthalazin-1(2H)-one

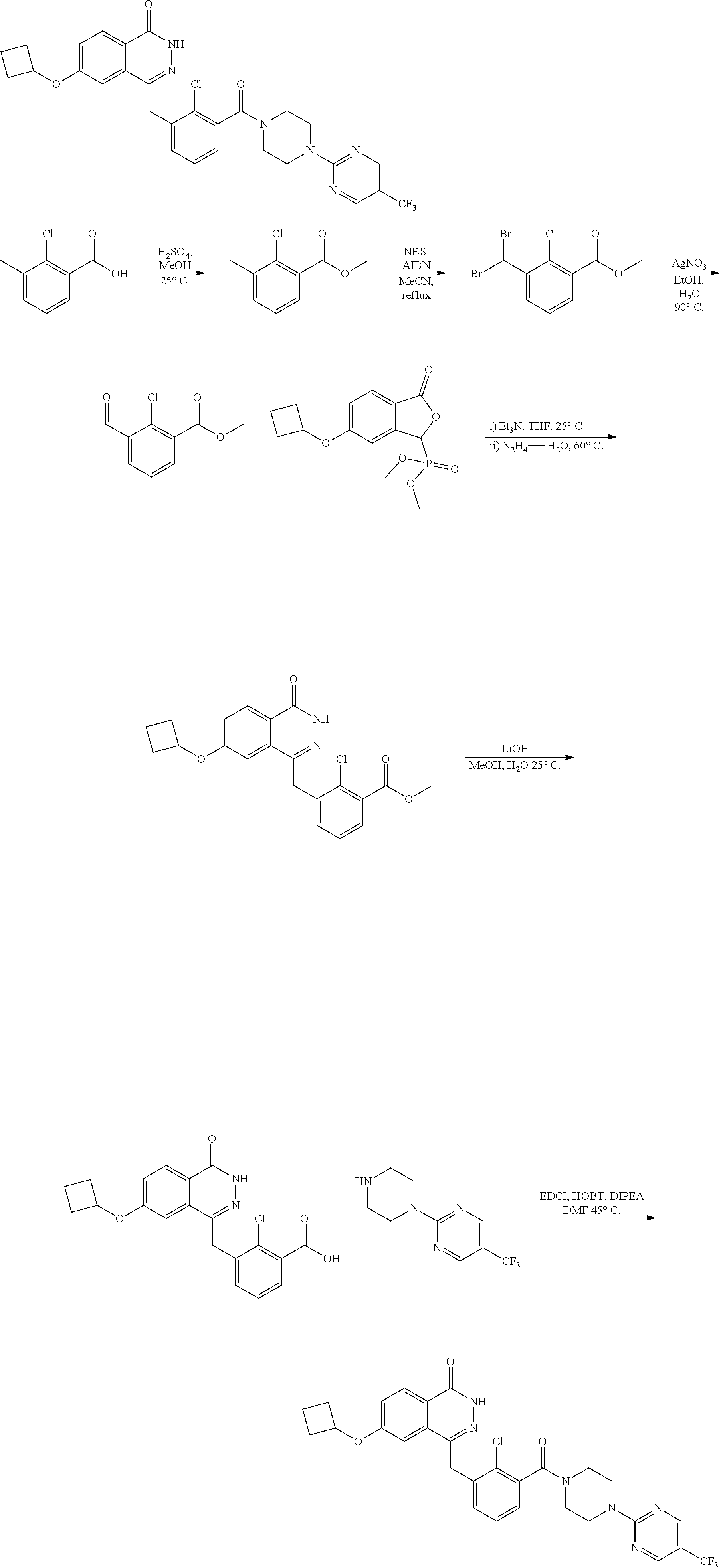

### Step 1

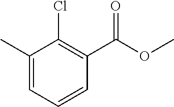

### Methyl 2-chloro-3-methylbenzoate

To a solution of 2-chloro-3-methylbenzoic acid (5.0) g, 29.31 mmol) in MeOH (15 mL) was added conc. H₂SO₄ (6.25 mL) in portions. The mixture was stirred at 25° C. for 20 hours. The solution was concentrated in vacuo. This residue was diluted with water (30 mL) and extracted with EtOAc (20 mL×3). The combined organic layer was washed with brine, dried over anhydrous Na₂SO₄, filtered and concentrated in vacuo to give methyl 2-chloro-3-methylbenzoate (5.20 g, 96% yield) as a white solid. LCMS ESI m/z: 185 [M+H]⁺. This was used without further purification.

### Step 2

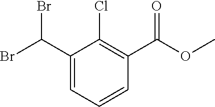

### Methyl 2-chloro-3-(dibromomethyl)benzoate

A mixture of methyl 2-chloro-3-methylbenzoate (500 mg, 2.71 mmol), NBS (1.59 g, 8.94 mmol) and AIBN (133 mg, 0.81 mmol) in ACN (10 mL) was stirred under reflux for 16 hours. The mixture was cooled to rt, diluted with H₂O (30 mL), and extracted with DCM (30 mL×3). The combined organic layer was dried over anhydrous Na₂SO₄, filtered and concentrated in vacuo. The residue was purified by silica gel chromatography (EtOAc/petroleum ether=0% to 15%) to obtain methyl 2-chloro-3-(dibromomethyl)benzoate (797 mg, 86% yield) as a white solid. LCMS ESI m/z: 342/344 [M+H]⁺.

### Step 3

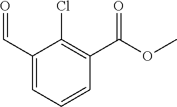

### Methyl 2-chloro-3-formylbenzoate

To a solution of methyl 2-chloro-3-(dibromomethyl)benzoate (700 mg, 2.04 mmol) in EtOH (10 mL), was added AgNO₃ (3.47 g, 9.58 mmol) and H₂O (5 mL). The mixture was stirred at 80° C. for 30 minutes. The solution was concentrated in vacuo, diluted with water (30 mL) and extracted with EtOAc (20 mL×3). The combined organic layer was washed with brine, dried over anhydrous Na₂SO₄, filtered and concentrated in vacuo to give methyl 2-chloro-3-formylbenzoate (370 mg, 91% yield) as a yellow solid. LCMS ESI m/z: 199 [M+H]⁺. This was used without further purification.

### Step 4

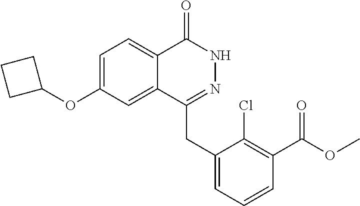 [SMILES: COC(=O)c1cccc(Cc2n[nH]c(=O)c3ccc(OC4CCC4)cc23)c1Cl]

### Methyl 2-chloro-3-((7-cyclobutoxy-4-oxo-3,4-dihydrophthalazin-1-yl)methyl) benzoate

A solution of dimethyl (6-cyclobutoxy-3-oxo-1,3-dihydroisobenzofuran-1-yl)phosphonate (200 mg, 0.64 mmol), methyl 2-chloro-3-formylbenzoate (134 mg, 0.67 mmol) and Et₃N (194 mg, 1.92 mmol) in anhydrous THF (6 mL) was stirred at 25° C. under Ar (g) for 3 hours. Hydrazine hydrate (49 mg, 0.83 mmol) was added and the reaction was stirred at 70° C. for 2 hours. The reaction was cooled to rt, then THF was removed under vacuum. The formed solids were washed with water (10 mL) and petroleum ether/EtOAc (10:1, 10 mL) to give methyl 2-chloro-3-((7-cyclobutoxy-4-oxo-3,4-dihydrophthalazin-1-yl)methyl)benzoate (96 mg, 38% yield) as a white solid. LCMS EST m/z: 399 [M+H]⁺.

### Step 5

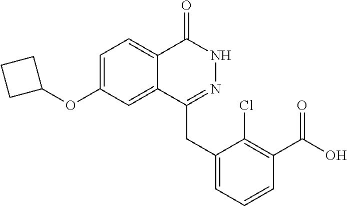 [SMILES: O=C(O)c1cccc(Cc2n[nH]c(=O)c3ccc(OC4CCC4)cc23)c1Cl]

### 2-chloro-3-((7-cyclobutoxy-4-oxo-3,4-dihydrophthalazin-1-yl)methyl)benzoic acid

A solution of methyl 2-chloro-3-((7-cyclobutoxy-4-oxo-3,4-dihydrophthalazin-1-yl)methyl)benzoate (96 mg, 0.24 mmol) and LiOH (60 mg, 2.51 mmol) in MeOH (3 mL) and H₂O (1 mL) was stirred at rt for 1 hour. The organic solvent was removed in vacuum and aq HCl (1N) was added until the solution reached to pH 5˜6. The solids were collected by filtration, washed with water and dried under vacuum to give 2-chloro-3-((7-cyclobutoxy-4-oxo-3,4-dihydrophthalazin-1-yl)methyl)benzoic acid (90 mg, 93% yield) as a white solid. LCMS ESI m/z: 384 [M+H]⁺. This was used without further purification.

### Step 6

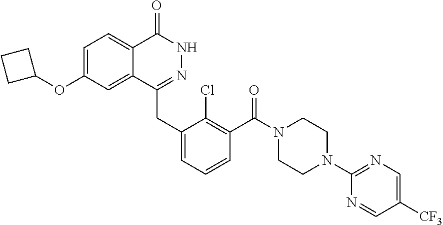 [SMILES: O=C(c1cccc(Cc2n[nH]c(=O)c3ccc(OC4CCC4)cc23)c1Cl)N1CCN(c2ncc(C(F)(F)F)cn2)CC1]

### 4-(2-Chloro-3-(4-(5-(trifluoromethyl)pyrimidin-2-yl)piperazine-1-carbonyl)benzyl)-6-cyclobutoxyphthalazin-1(2H)-one

To a solution of 2-(piperazin-1-yl)-5-(trifluoromethyl)pyrimidine (92 mg, 0.34 mmol) and 5-((7-2-chloro-3-((7-cyclobutoxy-4-oxo-3,4-dihydrophthalazin-1-yl)methyl)benzoic acid (120 mg, 0.31 mmol) in DMF (3 mL) was added EDCI (90 mg, 0.47 mmol), HOBt (63 mg, 0.47 mmol) and DIPEA (201 mg, 1.56 mmol). The reaction mixture was stirred at 45° C. for 1 hour. The reaction was purified by prep-HPLC to give the 4-(2-chloro-3-(4-(5-(trifluoromethyl)pyrimidin-2-yl)piperazine-1-carbonyl)benzyl)-6-cyclobutoxyphthalazin-1(2H)-one (37 mg, 20% yield) as a white solid. LCMS ESI m/z: 599 [M+H]⁺. ¹H-NMR (400 MHz, DMSO-d₆) δ 12.42 (s, 1H), 8.74 (s, 2H), 8.18 (d, J=8.8 Hz, 1H), 7.39-7.28 (m, 4H), 7.16 (d, J=2.3 Hz, 1H), 4.89 (p, J=7.1 Hz, 1H), 4.50-4.36 (m, 2H), 4.04-3.71 (m, 6H), 3.27 (L J=5.1 Hz, 2H), 2.49-2.41 (m, 2H), 2.14-1.98 (m, 2H), 1.88-1.59 (m, 2H).

### Synthesis of Example 172: 6-Cyclobutoxy-4-(3-fluoro-4-(4-(5-(trifluoromethyl)pyrimidin-2-yl)piperazine-1-carbonyl)benzyl)phthalazin-1(2H)-one

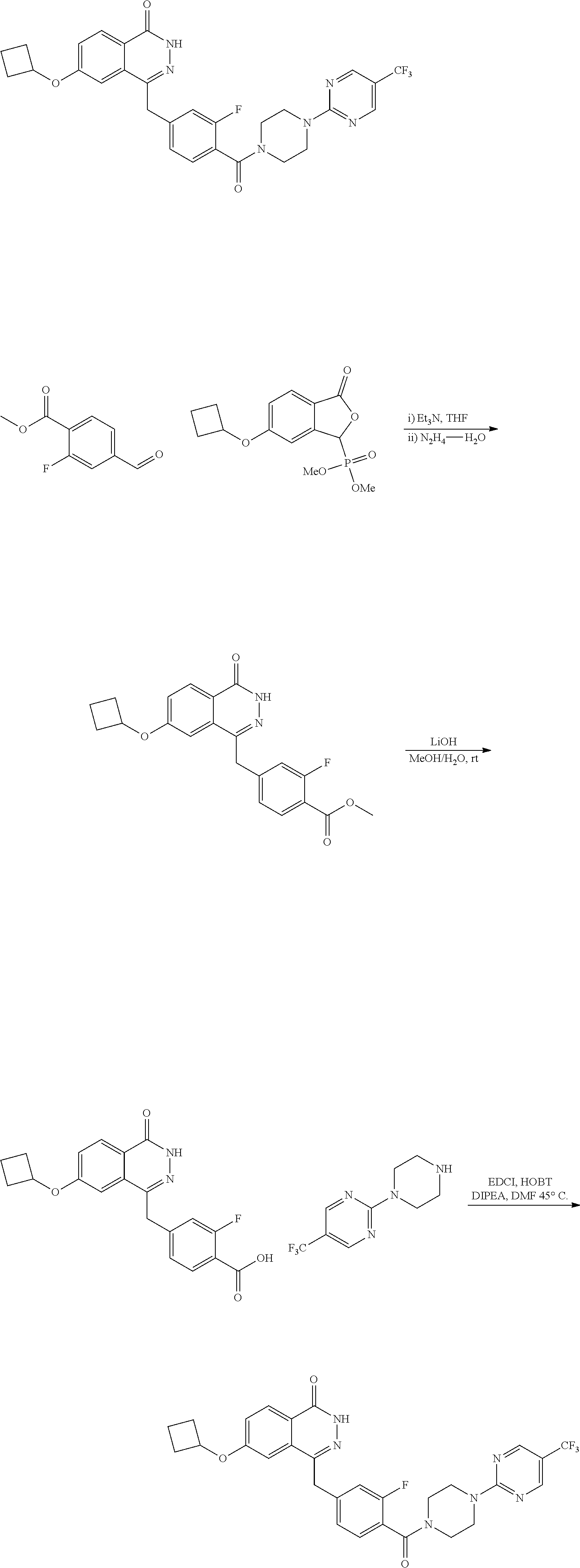

### Step 1

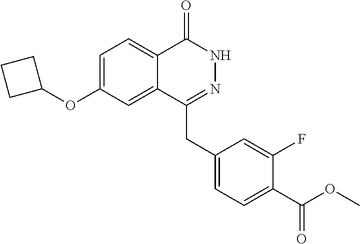 [SMILES: COC(=O)c1ccc(Cc2n[nH]c(=O)c3ccc(OC4CCC4)cc23)cc1F]

### Methyl 4-((7-cyclobutoxy-4-oxo-3,4-dihydrophthalazin-1-yl)methyl)-2-fluorobenzoate

A solution of methyl 2-fluoro-4-formylbenzoate (150 mg, 0.82 mmol), dimethyl (6-bromo-3-oxo-1,3-dihydroisobenzofuran-1-yl)phosphonate (257 mg, 0.82 mmol) and Et₃N (250 mg, 2.47 mmol) in anhydrous THF (10 mL) was stirred at 25° C. under Ar (g) for 3 hours. Hydrazine hydrate (83 mg, 1.66 mmol) was added and the reaction was stirred at 70° C. for 2 hours. The reaction was cooled to rt, then THF was removed under vacuum. The formed solids were washed with water (10 mL) and concentrated under reduced pressure to give 6-(4-(3-formylbenzoyl)piperazin-1-yl)nicotinonitrile (209 mg, 66% yield) as a white solid. LCMS ESI m/z: 383.0 [M+H]⁺. This was used without further purification.

### Step 2

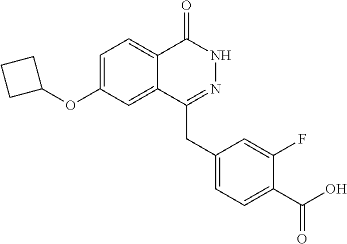 [SMILES: O=C(O)c1ccc(Cc2n[nH]c(=O)c3ccc(OC4CCC4)cc23)cc1F]

### 4-((7-Cyclobutoxy-4-oxo-3,4-dihydrophthalazin-1-yl)methyl)-2-fluorobenzoic acid

A mixture of methyl 4-((7-cyclobutoxy-4-oxo-3,4-dihydrophthalazin-1-yl)methyl)-2-fluorobenzoate (209 mg, 0.55 mmol) and LiOH (131 mg, 5.47 mmol) in MeOH (5 mL) and H₂O (5 mL) was stirred at rt for 16 hours. The organic solvent was removed in vacuo, aq 2N HCl was added until the solution reached pH 5˜6, then extracted with EtOAc (30 mL×3). The combined organic layer was washed with brine, dried over anhydrous Na₂SO₄, filtered and concentrated in vacuo to give 4-((7-cyclobutoxy-4-oxo-3,4-dihydrophthalazin-1-yl)methyl)-2-fluorobenzoic acid (200 mg, 99% yield) as a white solid. LCMS EST m/z: 369.0 [M+H]⁺. This was used without further purification.

### Step 3

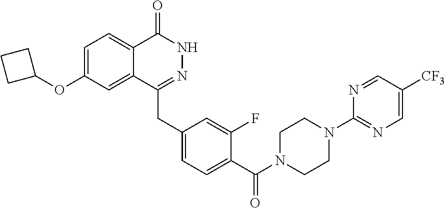 [SMILES: O=C(c1ccc(Cc2n[nH]c(=O)c3ccc(OC4CCC4)cc23)cc1F)N1CCN(c2ncc(C(F)(F)F)cn2)CC1]

### 6-Cyclobutoxy-4-(3-fluoro-4-(4-(5-(trifluoromethyl)pyrimidin-2-yl)piperazine-1-carbonyl)benzyl)phthalazin-1(2H)-one

To a solution of 4-((7-cyclobutoxy-4-oxo-3,4-dihydrophthalazin-1-yl)methyl)-2-fluorobenzoic acid (100 mg, 0.27 mmol) and 2-(piperazin-1-yl)-5-(trifluoromethyl)pyrimidine (54 mg, 0.27 mmol) in DMF (3 mL) was added HOBt (55 mg, 0.41 mmol). EDCI (78 mg, 0.41 mmol) and DIPEA (105 mg, 0.81 mmol). The reaction mixture was stirred at rt for 16 hours. The reaction was purified by prep-HPLC to give 6-cyclobutoxy-4-(3-fluoro-4-(4-(5-(trifluoromethyl)pyrimidin-2-yl)piperazine-1-carbonyl)benzyl)phthalazin-1(2H)-one (41.1 mg, 26% yield) as a white solid. LCMS EST m/z: 583.2 [M+H]⁺. ¹H-NMR (400 MHz, DMSO-d₆) δ 12.48 (s, 1H), 8.74 (s, 2H), 8.17 (d, J=8.8 Hz, 1H), 7.39 (t, J=7.5 Hz, 1H), 7.33 (d, J=2.0 Hz, 1H), 7.31 (d, J=2.3 Hz, 1H), 7.23 (d, J=7.8 Hz, 1H), 7.11 (d, J=2.3 Hz, 1H), 4.86 (dd, J=14.2, 7.1 Hz, 1H), 4.35 (s, 2H), 3.92 (d, J=5.4 Hz, 2H), 3.80 (d, J=5.2 Hz, 2H), 3.74 (d, J=5.0 Hz, 2H), 3.31 (s, 2H), 2.43-2.30 (m, 2H), 2.07-1.95 (m, 2H), 1.80 (dd, J=20.2, 10.1 Hz, 1H), 1.66 (dd, J=18.5, 8.4 Hz, 1H).

### Synthesis of Example 173: 4-(2-Chloro-5-(4-(5-(trifluoromethyl)pyrimidin-2-yl)piperazine-1-carbonyl)benzyl)-6-cyclobutoxyphthalazin-1(2H)-one

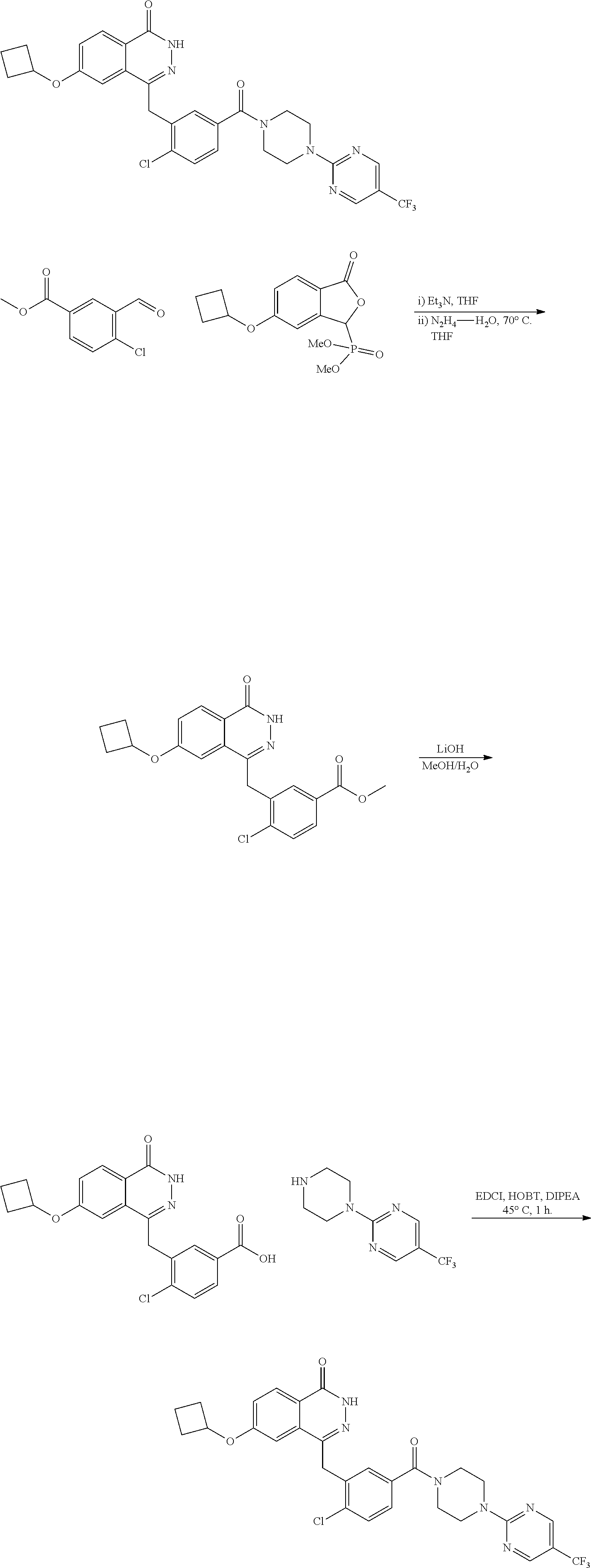

### Step 1

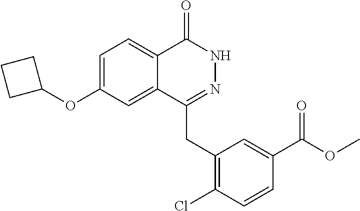 [SMILES: COC(=O)c1ccc(Cl)c(Cc2n[nH]c(=O)c3ccc(OC4CCC4)cc23)c1]

### methyl 4-chloro-3-((7-cyclobutoxy-4-oxo-3,4-dihydrophthalazin-1-yl)methyl) benzoate

A solution of dimethyl (6-bromo-3-oxo-1,3-dihydroisobenzofuran-1-yl)phosphonate (200 mg, 0.64 mmol), methyl 4-chloro-3-formylbenzoate (127 mg, 0.64 mmol) and Et₃N (194 mg, 1.92 mmol) in anhydrous THF (5 mL) was stirred at 25° C. under Ar (g) for 15 hours. Hydrazine hydrate (49 mg, 0.83 mmol) was added and the reaction was stirred at 70° C. for 1 hour. The reaction was cooled to rt, then THF was removed under vacuum. The formed solid was washed with water (10 mL) and petroleum ether/EA (2:1, 10 mL) to give methyl 4-chloro-3-((7-cyclobutoxy-4-oxo-3,4-dihydrophthalazin-1-yl)methyl)benzoate (140 mg, 55% yield) as a white solid. LCMS ESI m/z: 399 [M+H]⁺.

### Step 2

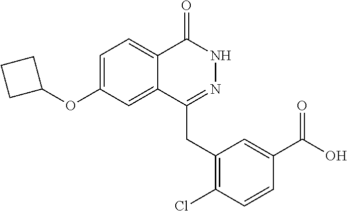 [SMILES: O=C(O)c1ccc(Cl)c(Cc2n[nH]c(=O)c3ccc(OC4CCC4)cc23)c1]

### 4-Chloro-3-((7-cyclobutoxy-4-oxo-3,4-dihydrophthalazin-1-yl)methyl)benzoic acid

A solution of methyl 4-chloro-3-((7-cyclobutoxy-4-oxo-3,4-dihydrophthalazin-1-yl)methyl)benzoate (140 mg, 0.35 mmol) and LiOH (84 mg, 3.5 mmol) in MeOH (3 mL) and H₂O (1 mL) was stirred at rt for 2 hours. The organic solvent was removed in vacuo and aq HCl (1 M) was added until the solution reached pH 5˜6. The solids were collected by filtration, washed with water and dried under vacuum to give 4-chloro-3-((7-cyclobutoxy-4-oxo-3,4-dihydrophthalazin-1-yl)methyl)benzoic acid (116 mg, 86% yield) as a white solid. LCMS ESI m/z: 385 [M+H]⁺.

### Step 3

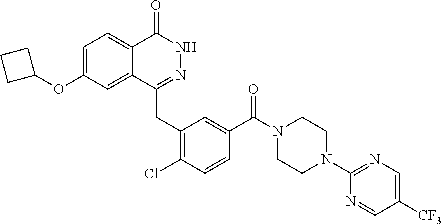 [SMILES: O=C(c1ccc(Cl)c(Cc2n[nH]c(=O)c3ccc(OC4CCC4)cc23)c1)N1CCN(c2ncc(C(F)(F)F)cn2)CC1]

### 4-(2-chloro-5-(4-(5-(trifluoromethyl)pyrimidin-2-yl)piperazine-1-carbonyl)benzyl)-6-cyclobutoxyphthalazin-1(2H)-one

To a solution of 4-chloro-3-((7-cyclobutoxy-4-oxo-3,4-dihydrophthalazin-1-yl)methyl)benzoic acid (116 mg, 0.30 mmol) and 2-(piperazin-1-yl)-5-(trifluoromethyl)pyrimidine (89 mg, 0.33 mmol) in DMF (2 mL) was added EDCI (87 mg, 0.45 mmol). HOBt (61 mg, 0.45 mmol) and DIPEA (195 mg, 1.50 mmol). The reaction mixture was stirred at 45° C. for 1 hour. The reaction was purified by prep-HPLC to give 4-(2-chloro-5-(4-(5-trifluoromethyl)pyrimidin-2-yl)piperazine-1-carbonyl) benzyl)-6-cyclobutoxyphthalazin-1(2H)-one (75 mg, 41% yield) as a white solid. LCMS ESI m/z: 599 [M+H]⁺. ¹H NMR (400 MHz, DMSO-d₆) δ 12.39 (s, 1H), 8.74 (s, 2H), 8.18 (d, J=8.8 Hz, 1H), 7.61-7.58 (m, 1H), 7.40-7.35 (m, 3H), 7.18-7.15 (m, 1H), 4.93-4.86 (m, 1H), 4.43 (s, 2H), 3.95-3.63 (m, 6H), 2.48-2.35 (m, 3H), 2.20-1.91 (m, 3H), 1.84-1.76 (m, 1H), 1.71-1.64 (m, 1H).

### Synthesis of Example 174: 4-(3-chloro-5-(4-(5-(trifluoromethyl)pyrimidin-2-yl)piperazine-1-carbonyl)benzyl)-6-cyclobutoxyphthalazin-1(2H)-one

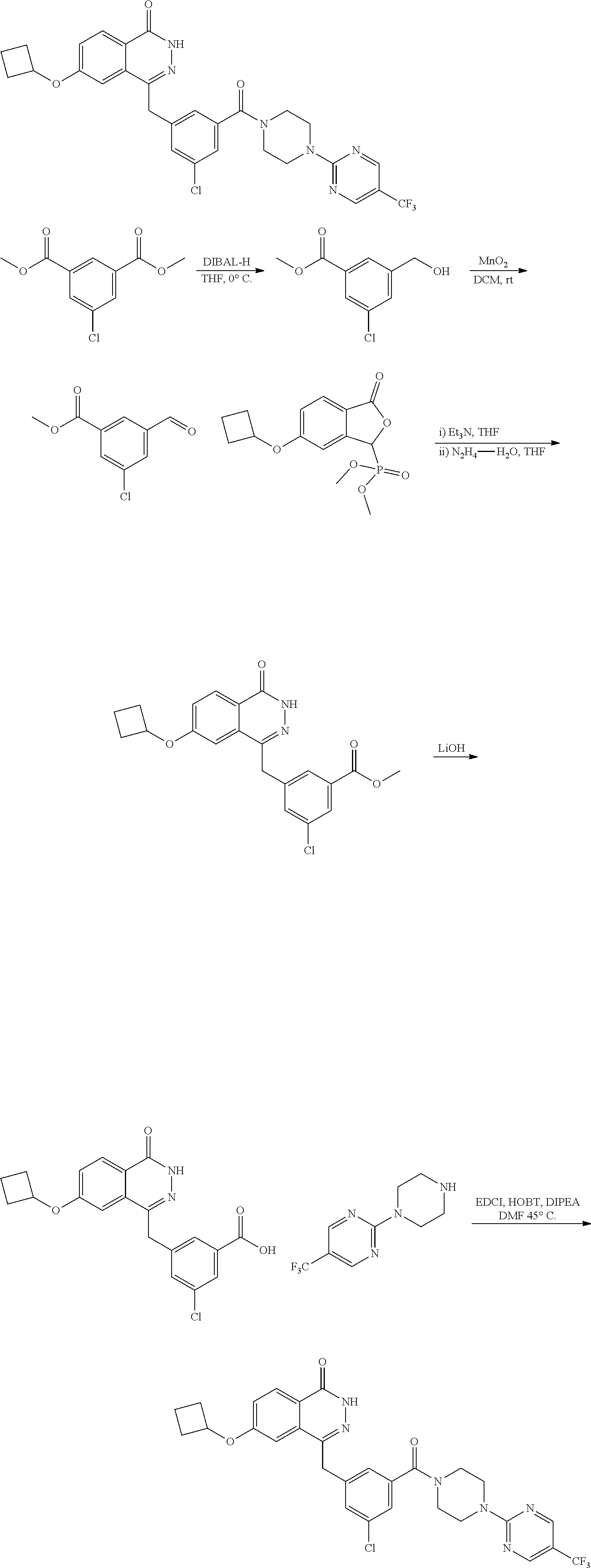

### Step 1

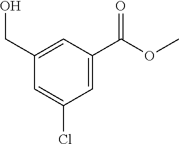

### Methyl 3-chloro-5-(hydroxymethyl)benzoate

To a solution of dimethyl 5-chloroisophthalate (500 mg, 2.19 mmol) in THF (10 mL) was added DIBAL-H (1M in THF, 6.6 ML, 6.6 mmol) at 0° C., then the mixture was stirred at rt for 30 minutes. The reaction was quenched with saturated aqueous solution of potassium sodium tartrate tetrahydrate (20 mL), then extracted with EtOAc (20 mL×3). The combined organic phase was washed with brine, dried over Na₂SO₄, filtered and concentrated under reduced pressure. The residue was purified by silica gel chromatography (Petroleum ether/EtOAc=3/1) to give methyl 3-chloro-5-(hydroxymethyl)benzoate (213 mg, 49% yield) as a colorless oil. LCMS ESI m/z: 201.1 [M+H]⁺.

### Step 2

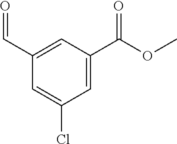

### Methyl 3-chloro-5-formylbenzoate

To a solution of methyl 3-chloro-5-(hydroxymethyl)benzoate (213 mg, 1.06 mmol) in DCM (5 mL) was added MnO₂ (1.38 g, 15.93 mmol), then the mixture was stirred at rt for 16 hours. The solid was removed by filtration and the filtrate was concentrated in vacuo to afford methyl 3-chloro-5-formylbenzoate (142 mg, 67% yield) as a colorless oil. LCMS ESI m/z: 199.0 [M+H]⁺.

### Step 3

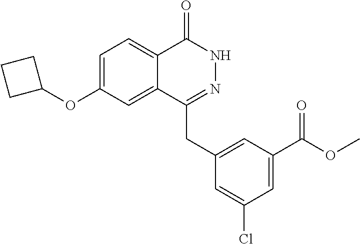 [SMILES: COC(=O)c1cc(Cl)cc(Cc2n[nH]c(=O)c3ccc(OC4CCC4)cc23)c1]

### Methyl 3-chloro-5-((7-cyclobutoxy-4-oxo-3,4-dihydrophthalazin-1-yl)methyl) benzoate

A solution of methyl 3-chloro-5-formylbenzoate (142 mg, 0.71 mmol), dimethyl (6-cyclobutoxy-3-oxo-1,3-dihydroisobenzofuran-1-yl)phosphonate (223 mg, 0.71 mmol) and Et₃N (217 mg, 2.14 mmol) in anhydrous THF (5 mL) was stirred at 45° C. under Ar (g) for 16 hours. N₂H₄—H₂O (72 mg, 1.43 mmol) was added and the reaction was stirred at 70° C. for 2 hours. The reaction was cooled to rt, then THF was removed under vacuum. The formed solids were washed with water (10 mL) and concentrated under reduced pressure to give methyl 3-chloro-5-(7-cyclobutoxy-4-oxo-3,4-dihydrophthalazin-1-yl)methyl)benzoate (155 mg, 54% yield) as a white solid. LCMS ESI m/z: 399.1 [M+H]⁺. This was used without further purification.

### Step 4

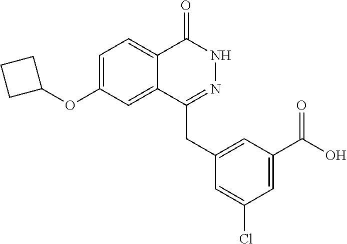 [SMILES: O=C(O)c1cc(Cl)cc(Cc2n[nH]c(=O)c3ccc(OC4CCC4)cc23)c1]

### 3-Chloro-5-((7-cyclobutoxy-4-oxo-3,4-dihydrophthalazin-1-yl)methyl)benzoic acid

A mixture of methyl 3-chloro-5-((7-cyclobutoxy-4-oxo-3,4-dihydrophthalazin-1-yl)methyl)benzoate (155 mg, 0.39 mmol) and LIOH (93 mg, 3.89 mmol) in MeOH (3 mL) and H₂O (3 mL) was stirred at rt for 3 hours. The organic solvent was removed in vacuo and aq HCl (2N) was added until the solution reached pH 5˜6, then extracted with EtOAc (30 mL×3). The combined organic layer was washed with brine, dried over anhydrous Na₂SO₄, filtered and concentrated in vacuo to give 3-chloro-5-((7-cyclobutoxy-4-oxo-3,4-dihydrophthalazin-1-yl)methyl)benzoic acid (137 mg, 92% yield) as a white solid. LCMS ESI m/z: 385.1 [M+H]⁺. This was used without further purification.

### Step 5

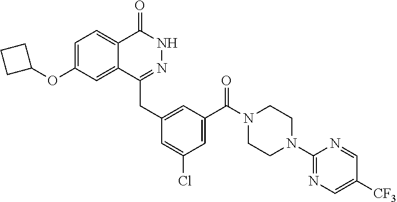 [SMILES: O=C(c1cc(Cl)cc(Cc2n[nH]c(=O)c3ccc(OC4CCC4)cc23)c1)N1CCN(c2ncc(C(F)(F)F)cn2)CC1]

### 4-(3-Chloro-5-(4-(5-(trifluoromethyl)pyrimidin-2-yl)piperazine-1-carbonyl)benzyl)-6-cyclobutoxyphthalazin-1(2H)-one

To a solution of 3-chloro-5-((7-cyclobutoxy-4-oxo-3,4-dihydrophthalazin-1-yl)methyl)benzoic acid (137 mg, 0.36 mmol) and 2-piperazin-1-yl)-5-(trifluoromethyl)pyrimidine (83 mg, 0.36 mmol) in DMF (3 mL) was added EDCI (102 mg, 0.53 mmol). HOBt (72 mg, 0.53 mmol) and DIPEA (138 mg, 1.07 mmol). The reaction mixture was stirred at rt for 16 hours. The reaction was purified by prep-HPLC to give 4-(3-chloro-5-(4-(5-(trifluoromethyl)pyrimidin-2-yl)piperazine-1-carbonyl)benzyl)-6-cyclobutoxyphthalazin-1(2H)-one (93 mg, 44% yield) as a white solid. LCMS ESI m/z: 599.0 [M+H]⁺. ¹H-NMR (400 MHz, CDCl₃) δ 12.47 (s, 1H), 8.74 (s, 2H), 8.16 (d, J=8.8 Hz, 1H), 7.54 (s, 1H), 7.38 (d, J=1.7 Hz, 1H), 7.36-7.30 (m, 2H), 7.13 (d, J=2.3 Hz, 1H), 4.87 (p, J=7.0 Hz, 1H), 4.36 (s, 2H), 4.03-3.61 (m, 6f), 3.36 (s, 1H), 3.31 (s, 1H), 2.44-2.35 (m, 2H), 2.08-1.94 (m, 2H), 1.79 (dd, J=20.1, 10.2 Hz, 1H), 1.66 (dd, J=18.5, 8.3 Hz, 1H).

### Synthesis of Example 175: 2-(4-(5-((7-cyclobutoxy-4-oxo-3,4-dihydrophthalazin-1-yl)methyl)-2,3-dihydrobenzofuran-7-carbonyl)piperazin-1-yl)isonicotinonitrile

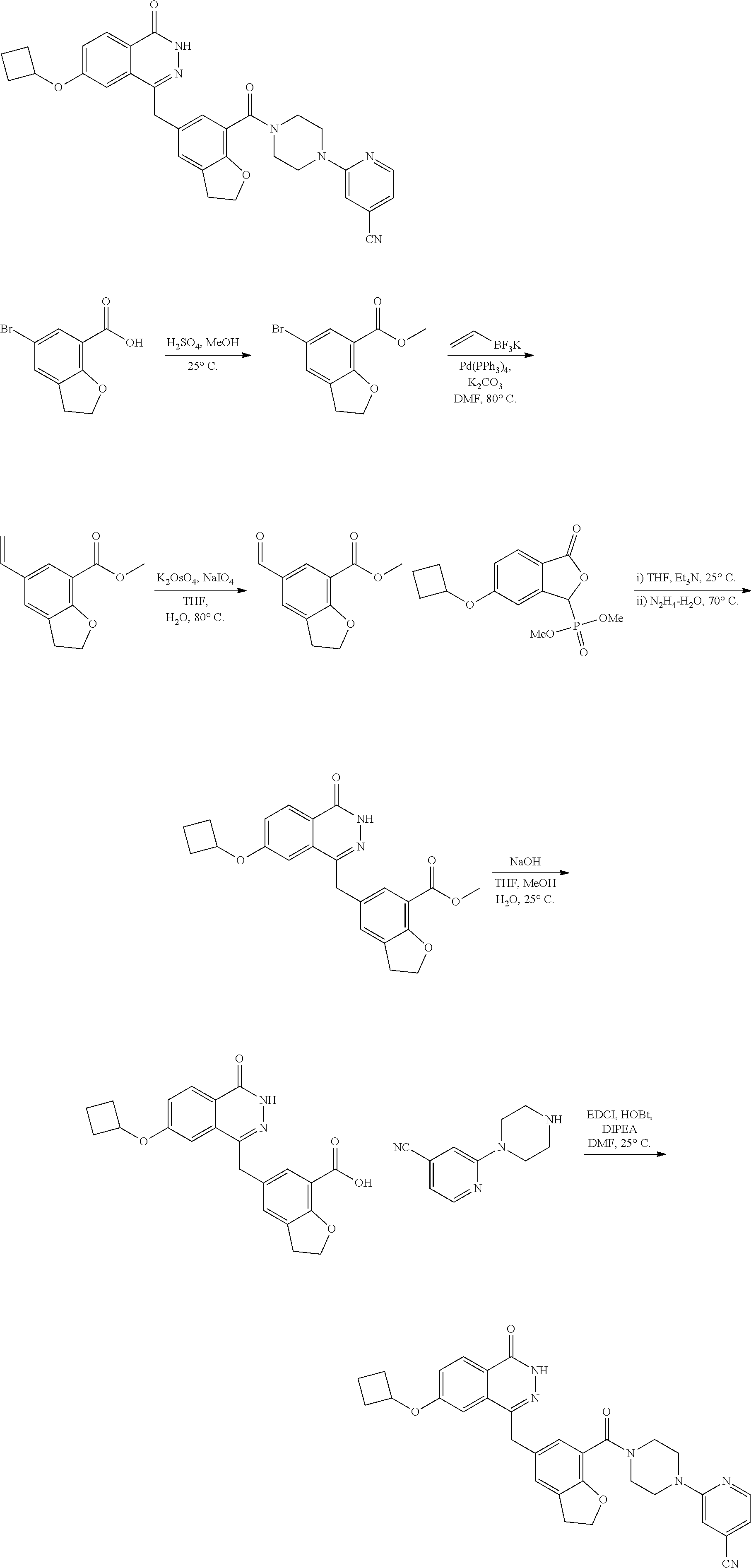

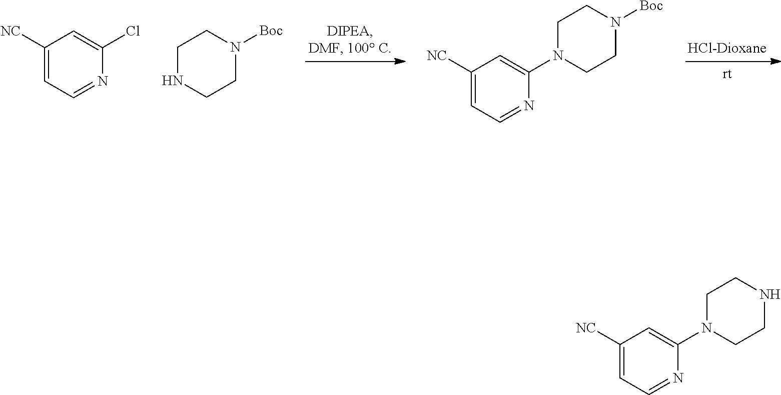 [SMILES: N#Cc1ccnc(Cl)c1 ; CC(C)(C)OC(=O)N1CCNCC1 ; CC(C)(C)OC(=O)N1CCN(c2cc(C#N)ccn2)CC1 ; N#Cc1ccnc(N2CCNCC2)c1]

### Step 1

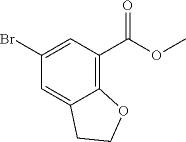

### Methyl 5-bromo-2,3-dihydrobenzofuran-7-carboxylate

To a solution of 5-bromo-2,3-dihydrobenzofuran-7-carboxylic acid (1.00 g, 4.11 mmol) in MeOH (5 mL) was added conc. H₂SO₄ (0.87 mL) in portions. The mixture was stirred at 25° C. for 20 hours. The solution was concentrated in vacuo. This residue was diluted with water (30 mL) and extracted with EtOAc (20 ml×3). The combined organic layer was washed with brine, dried over anhydrous Na₂SO₄, filtered and concentrated in vacuo to give methyl 5-bromo-2,3-dihydrobenzofuran-7-carboxylate (1.20 g, 99% yield) as a colorless oil. LCMS EST m/z: 257 [M+H]⁺. This was used without further purification.

### Step 2

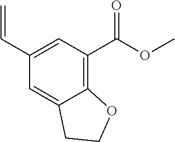

### Methyl 5-vinyl-2,3-dihydrobenzofuran-7-carboxylate

A mixture of 5-bromo-2,3-dihydrobenzofuran-7-carboxylate (1.2 g, 4.48 mmol), potassium vinyltrifluoroborate (603 mg, 4.50 mmol), K₂CO₃ (1.54 g, 11.20 mmol) and Pd(PPh₃)₄ (259 mg, 0.224 mmol) in DMF (20 mL) was stirred at 80° C. under Ar (g) for 4 hours. The mixture was cooled to rt, diluted with H₂O (20 mL) and extracted with EtOAc (30 mL×3). The combined organic layer was washed with brine, dried over anhydrous Na₂SO₄, filtered and concentrated in vacuo. The residue was purified by silica gel chromatography (EtOAc/petroleum ether=10 to 15%) to give methyl 5-vinyl-2,3-dihydrobenzofuran-7-carboxylate (807 mg, 89% yield) as a colorless oil. LCMS ESI m/z: 205 [M+H]⁺.

### Step 3

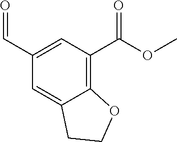

### Methyl 5-formyl-2,3-dihydrobenzofuran-7-carboxylate

To a mixture of methyl 5-vinyl-2,3-dihydrobenzofuran-7-carboxylate (607 mg, 2.76 mmol) in THF (8 mL) and H₂O (5 mL) was added K₂OsO₄-2H₂O (10 mg, 0.03 mmol). The mixture was stirred at room temperature for 5 min, then NaIO₄ (1.77 g, 8.29 mmol) was added and the mixture was stirred at 80° C. for 2 hours. The solids were removed by filtration. The filtrate was diluted with H₂O (5 mL) and extracted with EtOAc (30 mL×3). The combined organic layer was washed with brine, dried over anhydrous Na₂SO₄, filtered and concentrated in vacuo to give methyl 5-formyl-2,3-dihydrobenzofuran-7-carboxylate (284 mg, 49/o yield). LCMS ESI m/z: 207 [M+H]⁺. This was used without further purification.

### Step 4

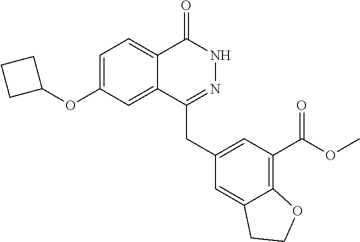 [SMILES: COC(=O)c1cc(Cc2n[nH]c(=O)c3ccc(OC4CCC4)cc23)cc2c1OCC2]

### Methyl 5-((7-cyclobutoxy-4-oxo-3,4-dihydrophthalazin-1-yl)methyl)-2,3-dihydrobenzofuran-7-carboxylate

A solution of dimethyl (6-cyclobutoxy-3-oxo-1,3-dihydroisobenzofuran-1-yl)phosphonate (200 mg, 0.97 mmol), methyl 5-formyl-2,3-dihydrobenzofuran-7-carboxylate (504 mg, 0.97 mmol) and Et₃N (294 mg, 2.91 mmol) in anhydrous THF (3 mL) was stirred at 25° C. under Ar (g) for 3 hours. Hydrazine hydrate (72 mg, 1.44 mmol) was added and the reaction was stirred at 70° C. for 2 hours. The reaction was cooled to rt, then THF was removed under vacuum. The formed solids were washed with water (10 mL) and petroleum ether/EtOAc (10:1, 10 mL) to give methyl 5-((7-cyclobutoxy-4-oxo-3,4-dihydrophthalazin-1-yl)methyl)-2,3-dihydrobenzofuran-7-carboxylate (130 mg, 37% yield) as a yellow solid. LCMS ESI m/z: 407 [M+H]⁺.

### Step 5

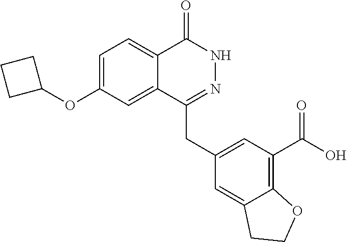 [SMILES: O=C(O)c1cc(Cc2n[nH]c(=O)c3ccc(OC4CCC4)cc23)cc2c1OCC2]

### 5-((7-Cyclobutoxy-4-oxo-3,4-dihydrophthalazin-1-yl)methyl)-2,3-dihydrobenzofuran-7-carboxylic acid

A solution of methyl 5-((7-cyclobutoxy-4-oxo-3,4-dihydrophthalazin-1-yl)methyl)-2,3-dihydrobenzofuran-7-carboxylate (130 mg, 0.37 mmol) and NaOH (147 mg, 3.69 mmol) in MeOH (2 mL). THF (2 mL) and H₂O (2 mL) was stirred at 45° C. for 1 hour. The organic solvent was removed in vacuum and aq HCl (1N) was added until the solution reached to pH 5˜6. The solids were collected by filtration, washed with water and dried under vacuum to give 5-((7-cyclobutoxy-4-oxo-3,4-dihydrophthalazin-1-yl)methyl)-2,3-dihydrobenzofuran-7-carboxylic acid (90 mg, 72% yield) as white solid. LCMS ESI m/z: 393 [M+H]⁺.

### Step 6

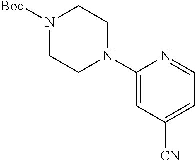 [SMILES: CC(C)(C)OC(=O)N1CCN(c2cc(C#N)ccn2)CC1]

### tert-Butyl 4-(4-cyanopyridin-2-yl)piperazine-1-carboxylate

A mixture of 2-chloroisonicotinonitrile (300 mg, 2.17 mmol), tert-butyl piperazine-1-carboxylate (403 mg, 2.17 mmol) and DIPEA (1.12 g, 8.66 mmol) in DMF (10 mL) was stirred at 100° C. for 20 hours. The reaction was diluted with water (30 mL) and extracted with EtOAc (20 mL×3). The combined organic layer was washed with brine, dried over anhydrous Na₂SO₄, filtered and concentrated in vacuo. The residue was purified by silica gel chromatography (EtOAc/petroleum ether=0% to 10%) to give tert-butyl 44(4-cyanopyridin-2-yl)piperazine-1-carboxylate (340 mg, 45% yield) as a white solid. LCMS ESI m/z: 289 [M+H]⁺.

### Step 7

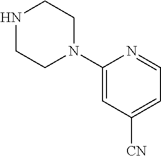 [SMILES: N#Cc1ccnc(N2CCNCC2)c1]

### 2-(Piperazin-1-yl)isonicotinonitrile

A solution of tert-butyl 4-(4-cyanopyridin-2-yl)piperazine-1-carboxylate (340 mg, 4.14 mmol) in HCl/dioxane (4M, 10 mL) was stirred at rt for 1 hour. The solvent was removed in vacuo to give 2-(piperazin-1-yl)isonicotinonitrile (609 mg, 85% yield) as a white solid. LCMS ESI m/z: 189 [M+H]⁺. This was used without further purification.

### Step 8

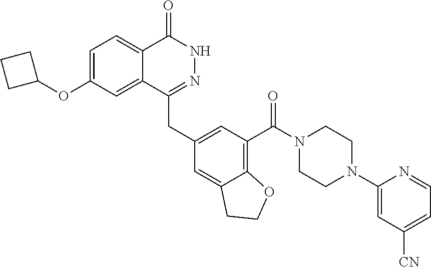 [SMILES: N#Cc1ccnc(N2CCN(C(=O)c3cc(Cc4n[nH]c(=O)c5ccc(OC6CCC6)cc45)cc4c3OCC4)CC2)c1]

### 2-(4-(5-((7-Cyclobutoxy-4-oxo-3,4-dihydrophthalazin-1-yl)methyl)-2,3-dihydrobenzofuran-7-carbonyl)piperazin-1-yl)isonicotinonitrile

To a solution of 2-(piperazin-1-yl)isonicotinonitrile (172 mg, 0.28 mmol) and 5-((7-cyclobutoxy-4-oxo-3,4-dihydrophthalazin-1-yl)methyl)-2,3-dihydrobenzofuran-7-carboxylic acid (90 mg, 0.23 mmol) in DMF (2 mL) was added EDCI (66 mg, 0.34 mmol). HOBt (47 mg, 0.34 mmol) and DIPEA (89 mg, 0.67 mmol). The reaction mixture was stirred at rt for 1 hour. The reaction was purified by prep-HPLC to give 2-(4-(5-((7-cyclobutoxy-4-oxo-3,4-dihydrophthalazin-1-yl)methyl)-2,3-dihydrobenzofuran-7-carbonyl)piperazin-1-yl)isonicotinonitrile (25 mg, 19% yield) as a white solid. LCMS ESI m/z: 562 [M+H]⁺. ¹H-NMR (400) MHz, DMSO-d₆) δ 12.44 (s, 1H), 8.31 (d, J=5.0 Hz, 1H), 8.14 (d, J=8.8 Hz, 1H), 7.34-7.27 (m, 2H), 7.23 (s, 1H), 7.14 (dd, 2H), 6.99 (dd, J=5.0 Hz, 1H), 4.84 (p, J=7.1 Hz, 1H), 4.53 (t, J=8.7 Hz, 2H), 4.21 (s, 2H), 3.73-3.49 (m, 6H), 3.30 (s, 2H), 3.15 (t, J=8.5 Hz, 2H), 2.47-2.34 (m, 2H), 2.06-1.93 (m, 2H), 1.86-1.59 (m, 2H).

### Synthesis of Example 176: 6-(4-(6-((7-Cyclobutoxy-4-oxo-3,4-dihydrophthalazin-1-yl)methyl)picolinoyl)piperazin-1-yl)nicotinonitrile

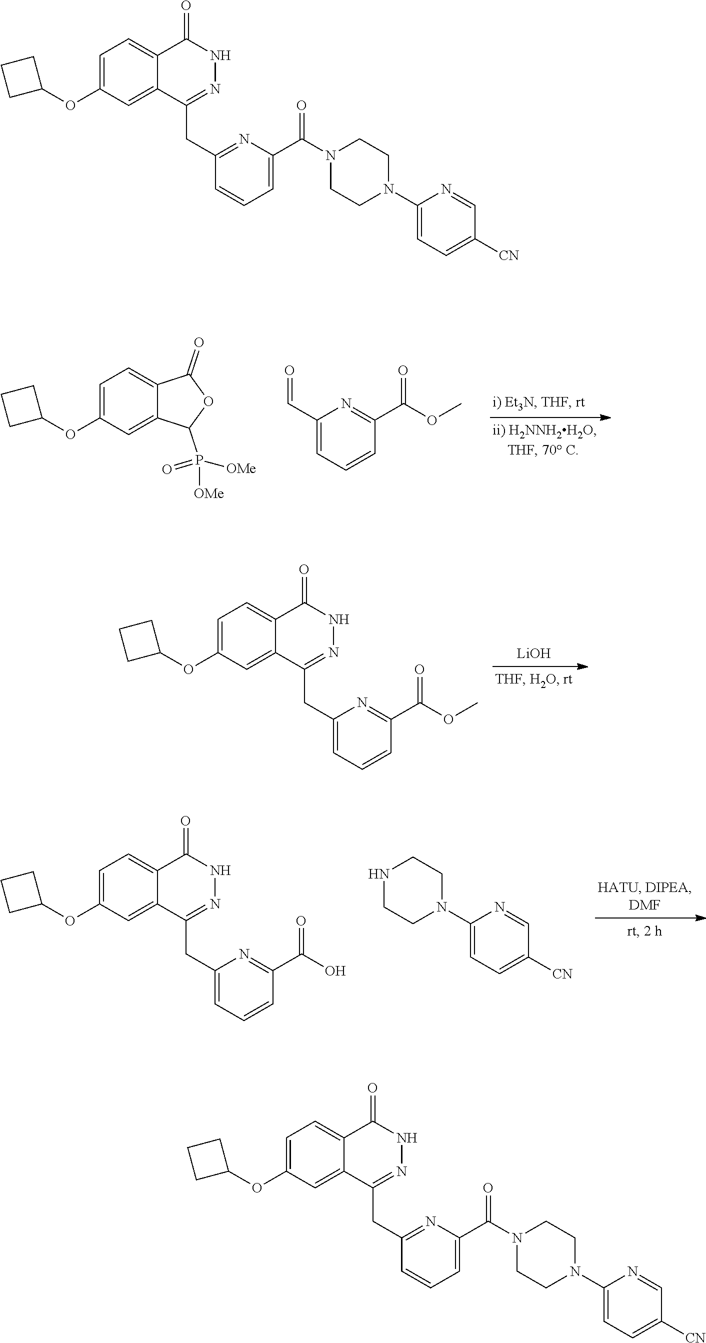

### Step 1

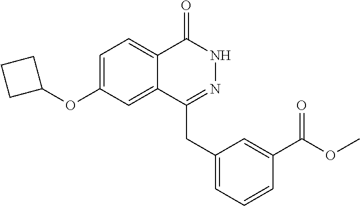 [SMILES: COC(=O)c1cccc(Cc2n[nH]c(=O)c3ccc(OC4CCC4)cc23)c1]

### Methyl 6-((7-cyclobutoxy-4-oxo-3,4-dihydrophthalazin-1-yl)methyl)picolinate

A solution of dimethyl (6-cyclobutoxy-3-oxo-1,3-dihydroisobenzofuran-1-yl)phosphonate (189 mg, 0.606 mmol), methyl 6-formyl-2-pyridinecarboxylate (100 mg, 0.606 mmol) and Et₃N (285 mg, 1.82 mmol) in anhydrous THF (3 mL) was stirred at 25° C. under Ar (g) for 15 hours. Hydrazine hydrate (72 mg, 1.44 mmol) was added and the reaction was stirred at 70° C. for 2 hours. The reaction was cooled to rt, then THF was removed under vacuum. The formed solids were washed with water (10 mL) and petroleum ether/EtOAc (2:1, 10 mL) to give methyl 6-((7-cyclobutoxy-4-oxo-3,4-dihydrophthalazin-1-yl)methyl)picolinate (221 mg, 85% yield) as a as a white solid. LCMS ESI m/z: 366.2 [M+H]⁺.

### Step 2

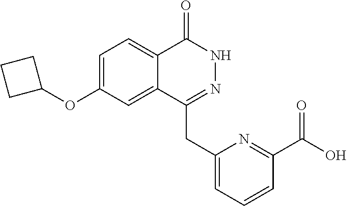 [SMILES: O=C(O)c1cccc(Cc2n[nH]c(=O)c3ccc(OC4CCC4)cc23)n1]

### 6-((7-Cyclobutoxy-4-oxo-3,4-dihydrophthalazin-1-yl)methyl)picolinic acid

A solution of methyl 6-((7-cyclobutoxy-4-oxo-3,4-dihydrophthalazin-1-yl)methyl)picolinate (221 mg, 606 umol), 1 M LiOH in H₂O (606 uL, 1.21 mmol) and THF (3.03 mL) was stirred at 25° C. under N₂ (g) for 15 hours. The reaction was stopped and the product was precipitated out of solution by addition of 10% citric acid in water until pH was found to be acidic (pH=2-3). Product precipitated as a bright yellow solid. Filtration over a Buchner and the solid was washed with water (2×20 mL), dried in vacuo. Used as is for the next step (150 mg, 65%). LCMS ESI m/z 352.1 (M+H)⁺. ¹H NMR (400 MHz, DMSO-d₆) δ 13.23 (s, 1H), 12.46 (s, 1H), 8.11 (d, J=8.8 Hz, 1H), 7.92-7.83 (m, 2H), 7.49 (dd, J=6.5, 2.3 Hz, 1H), 7.26 (dd, J=8.8, 2.4 Hz, 1H), 7.20 (d, J=2.3 Hz, 1H), 4.78 (p, J=7.1 Hz, 1H), 4.45 (s, 2H), 2.43-2.32 (m, 2H), 1.97 (ddd, J=12.2, 8.9, 2.5 Hz, 2H), 1.86-1.54 (m, 3H).

### Step 3

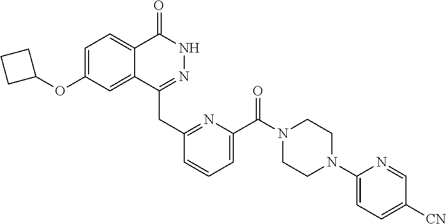 [SMILES: N#Cc1ccc(N2CCN(C(=O)c3cccc(Cc4n[nH]c(=O)c5ccc(OC6CCC6)cc45)n3)CC2)nc1]

### 6-(4-(6-((7-Cyclobutoxy-4-oxo-3,4-dihydrophthalazin-1-yl)methyl)picolinoyl)piperazin-1-yl)nicotinonitrile

A solution of 6-((7-cyclobutoxy-4-oxo-3,4-dihydrophthalazin-1-yl)methyl)picolinic acid (30.0 mg, 85.4 umol), 6-(piperazin-1-yl)nicotinonitrile (16.1 mg, 85.4 umol) and HATU (40.2 mg, 102 umol) in anhydrous DMF (854 uL) was stirred at 25° C. under N₂ (g) for 2 hours. The crude solution was purified by reverse phase chromatography (100% aqueous ammonium formate (AMF) solution (pH=3.8) to 70% MeCN in AMF). Fractions containing pure product were lyophilized overnight providing 6-(4-(6-((7-Cyclobutoxy-4-oxo-3,4-dihydrophthalazin-1-yl)methyl)picolinoyl)piperazin-1-yl)nicotinonitrile as a white solid (31.0 mg, 69% yield). LCMS ESI m/z 522.3 (M+H)⁺. 1H NMR (400 MHz, DMSO-d6) δ 12.45 (s, 1H), 8.50 (d, J=2.2 Hz, 1H), 8.14 (d, J=8.8 Hz, 1H), 7.90 (dd, J=9.4, 6.6 Hz, 2H), 7.52 (d, J=7.7 Hz, 1H), 7.48 (d, J=7.7 Hz, 1H), 7.29 (dd, J=8.8, 2.2 Hz, 1H), 7.08 (d, J=2.3 Hz, 1H), 6.84 (d, J=9.1 Hz, 1H), 4.78-4.67 (m, 1H), 4.46 (s, 2H), 3.72-3.64 (m, 4H), 3.29-3.21 (m, 2H), 2.35-2.27 (m, 2H), 1.99-1.86 (m, 2H), 1.78-1.52 (m, 2H).

### Synthesis of Example 177: 6-(4-(5-((7-Cyclobutoxy-4-oxo-3,4-dihydrophthalazin-1-yl)methyl)nicotinoyl)piperazin-1-yl)nicotinonitrile

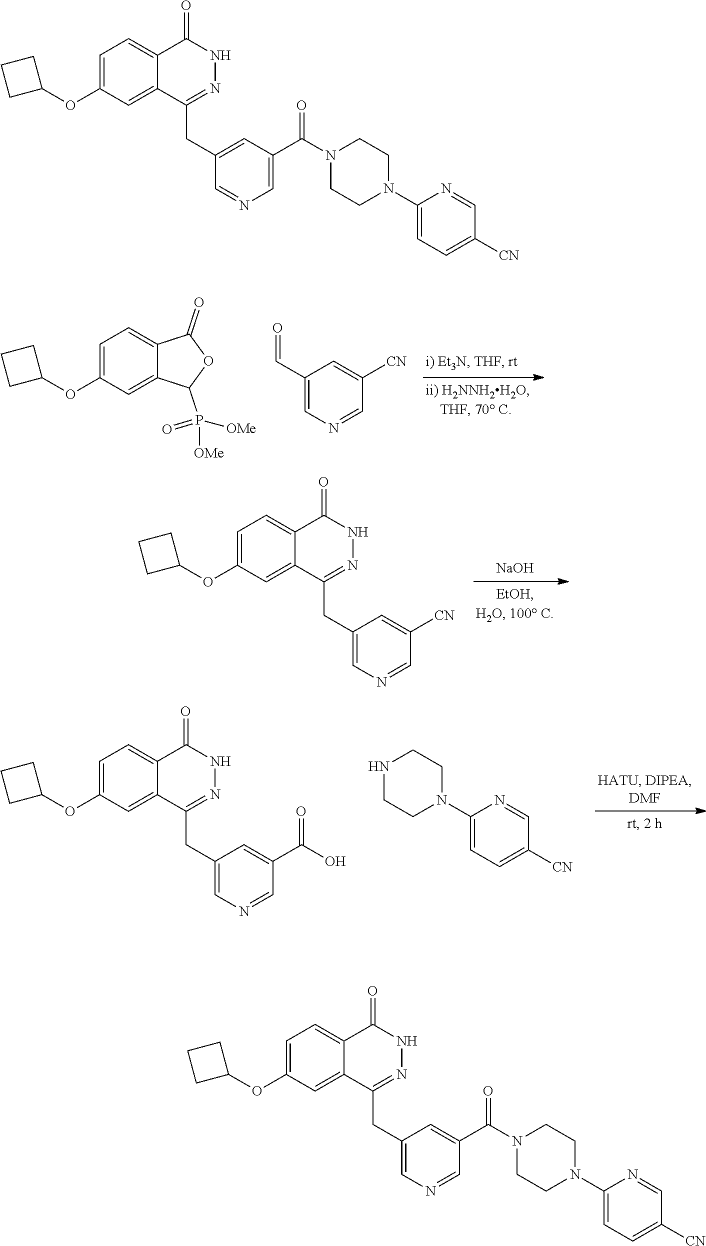

### Step 1

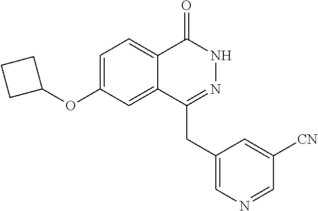 [SMILES: N#Cc1cncc(Cc2n[nH]c(=O)c3ccc(OC4CCC4)cc23)c1]

### 5-((7-Cyclobutoxy-4-oxo-3,4-dihydrophthalazin-1-yl)methyl)nicotinonitrile

A solution of dimethyl (6-cyclobutoxy-3-oxo-1,3-dihydroisobenzofuran-1-yl)phosphonate (355 mg, 1.14 mmol), 5-formylnicotinonitrile (150 mg, 1.14 mmol) and Et₃N (346 mg, 3.41 mmol) in anhydrous THF (5.68 mL) was stirred at 25° C. under Ar (g) for 15 hours. Hydrazine hydrate (72 mg, 2.27 mmol) was added and the reaction was stirred at 70° C. for 2 hours. The reaction was cooled to rt, then THF was removed under vacuum. The formed solids were washed with water (10 mL) and petroleum ether/EtOAc (2:1, 10 mL) to give 5-((7-cyclobutoxy-4-oxo-3,4-dihydrophthalazin-1-yl)methyl)nicotinonitrile (210 mg, 56% yield) as a as a white solid. ¹H NMR (400) MHz, cdcl3) δ 9.01 (d, J=2.1 Hz, 1H), 8.76 (d, J=1.9 Hz, 1H), 8.60 (t, J=2.1 Hz, 1H), 7.85 (dd, J=8.9, 6.6 Hz, 1H), 7.09-6.99 (m, 2H), 6.28 (s, 1H), 4.90-4.69 (m, 1H), 2.60-2.50 (m, 2H), 2.36-2.19 (m, 2H), 1.96 (d, J=10.4 Hz, 1H), 1.86-1.68 (m, 1H).

### Step 2

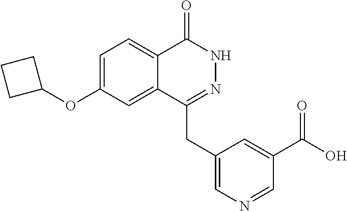 [SMILES: O=C(O)c1cncc(Cc2n[nH]c(=O)c3ccc(OC4CCC4)cc23)c1]

### 5-((7-Cyclobutoxy-4-oxo-3,4-dihydrophthalazin-1-yl)methyl)nicotinic acid

A solution of 5-((7-cyclobutoxy-4-oxo-3,4-dihydrophthalazin-1-yl)methyl)nicotinonitrile (200 mg, 0.602 mmol), an 8.0 M aqueous solution of sodium hydroxide (1.5 mL, 20 mmol), and Ethanol (3.02 mL) was stirred at 25° C. then heated to 100° C. for 1 hour. The reaction was cooled to rt, then the reaction was made acidic by addition of citric acid 10% in water (30 mL) until pH was found to be 2-3. The formed solid was triturated and filtered and dried in vacuo. The solid was then triturated again in in 40% EtOAc in 60% Heptanes (30 mL) for 30 minutes before being filtered on a Buchner. 5-((7-Cyclobutoxy-4-oxo-3,4-dihydrophthalazin-1-yl)methyl)nicotinic acid (170 mg, 80%) isolated as a white solid. LCMS ESI m/z: 352.2 [M+H]⁺. ¹H NMR (400 MHz, DMSO-d₆) δ 12.41 (s, 1H), 8.90 (d, J=2.0 Hz, 1H), 8.77 (d, J=2.1 Hz, 1H), 8.22-8.10 (m, 2H), 7.30 (dd, J=8.8, 2.3 Hz, 1H), 7.17 (d, J=2.3 Hz, 1H), 4.96-4.81 (m, 1H), 4.40 (s, 2H), 2.57-2.34 (m, 2H), 2.13-1.93 (m, 2H), 1.93-1.55 (m, 2H).

### Step 3

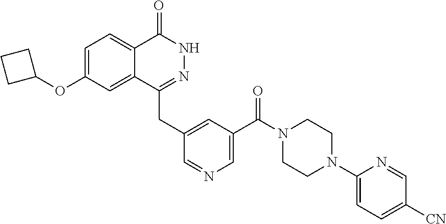 [SMILES: N#Cc1ccc(N2CCN(C(=O)c3cncc(Cc4n[nH]c(=O)c5ccc(OC6CCC6)cc45)c3)CC2)nc1]

### 6-(4-(5-((7-Cyclobutoxy-4-oxo-3,4-dihydrophthalazin-1-yl)methyl)nicotinoyl)piperazin-1-yl)nicotinonitrile

A solution of 5-((7-cyclobutoxy-4-oxo-3,4-dihydrophthalazin-1-yl)methyl)nicotinic acid (30.0 mg, 85.4 umol), 6-(piperazin-1-yl)nicotinonitrile (16.1 mg, 85.4 umol) and HATU (40.2 mg, 102 umol) in anhydrous DMF (854 uL) was stirred at 25° C. under N₂ (g) for 2 hours. The crude solution was purified by reverse phase chromatography (100% aqueous ammonium formate (AMF) solution (pH=3.8) to 70% MeCN in AMF). Fractions containing pure product were lyophilized overnight providing 6-(4-(5-((7-Cyclobutoxy-4-oxo-3,4-dihydrophthalazin-1-yl)methyl)nicotinoyl)piperazin-1-yl)nicotinonitrile as a white solid (30.5 mg, 68% yield). LCMS ESI m/z 522.3 (M+H)⁺. 1H NMR (400 MHz, DMSO-d₆) δ 12.43 (s, 1H), 8.64 (d, J=2.1 Hz, 1H), 8.50 (dd, J=4.4, 2.0 Hz, 2H), 8.14 (d, J=8.8 Hz, 1H), 7.88 (dd, J=9.1, 2.4 Hz, 1H), 7.76 (t, J=1.9 Hz, 1H), 7.32 (dd, J=8.8, 2.3 Hz, 1H), 7.15 (d, J=2.4 Hz, 1H), 6.90 (d, J=9.2 Hz, 1H), 4.87 (p, J=7.7 Hz, 1H), 4.37 (s, 2H), 3.80-3.58 (m, 6H), 3.42-3.34 (m, 2H), 2.41-2.34 (m, 2H), 2.03-1.95 (m, 2H), 1.82-1.55 (m, 2H).

### Synthesis of Example 178: 6-(4-(3-Bromo-((7-cyclobutoxy-4-oxo-3,4-dihydrophthalazin-1-yl)methyl)benzoyl)piperazin-1-yl)nicotinonitrile

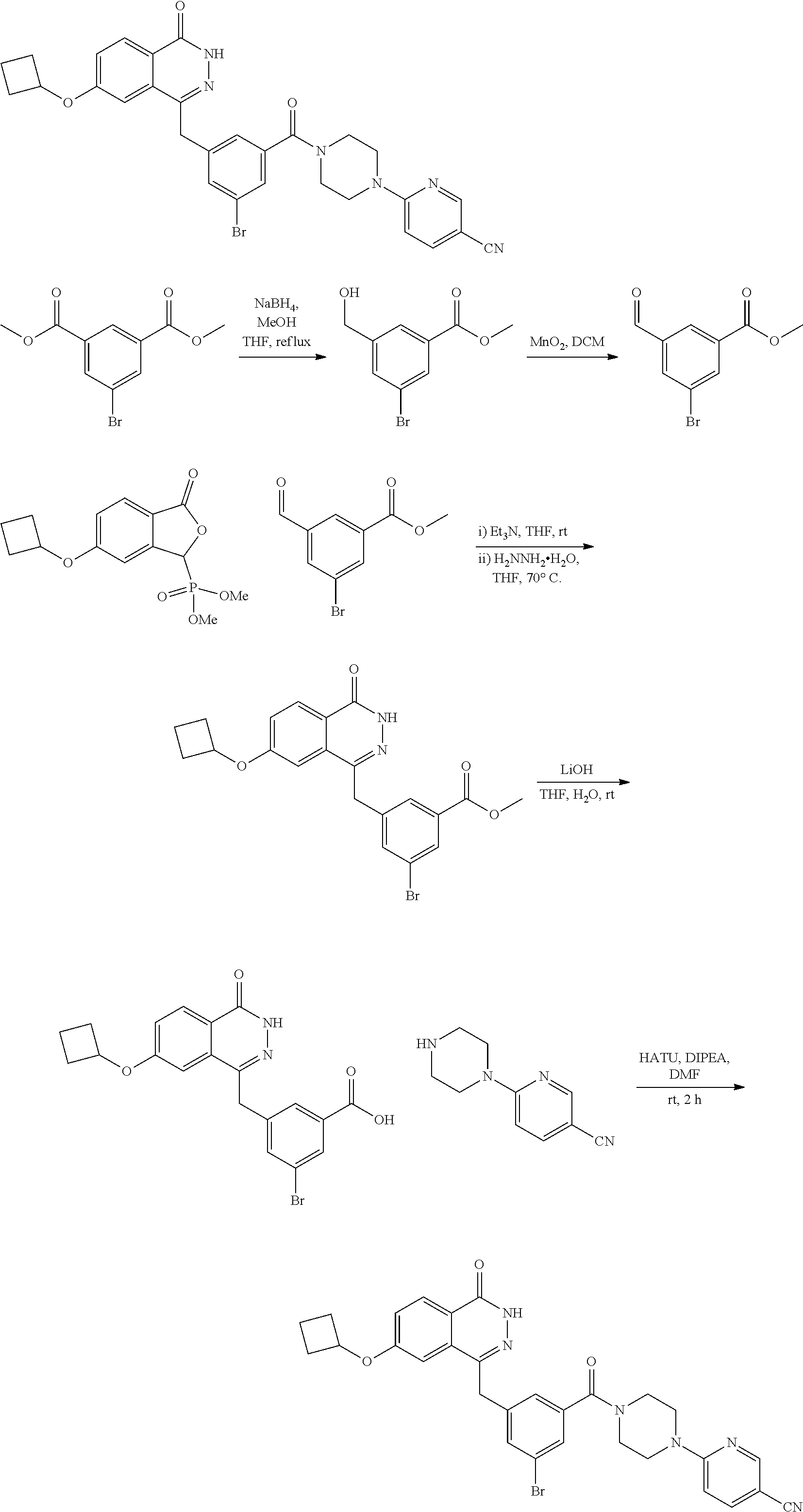

### Step 1

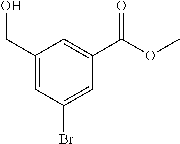

To a stirred solution of dimethyl 5-bromoisophthalate (5.4 g, 20 mmol) in methanol (300 mL) at 0° C. was slowly added sodium tetrahydroborate (10 g, 0.28 mol). The mixture was stirred at rt for 3 h, and then treated with water (300 mL). The volatiles were removed in vacuo and the aqueous phase was extracted with CH₂Cl₂ (100 mL×3). The combined organic layers were washed with brine, dried over anhydrous MgSO₄, and concentrated in vacuo. The resulting oil was purified by flash chromatography (100% Heptanes to 50% EtOAc in Heptanes) to afford the title compound, methyl 3-bromo-5-(hydroxymethyl)benzoate (1.50 g, 56%). ¹H NMR (400 MHz, CDCl₃) δ 8.08 (s, 1H), 7.94 (s, 1H), 7.73 (s, 1H), 4.74 (s, 2H), 3.92 (s, 3H).

### Step 2

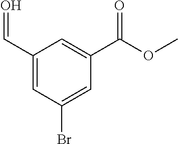

### Methyl 3-bromo-5-formylbenzoate

To a stirred solution of methyl 3-bromo-5-(hydroxymethyl)benzoate (1.35 g, 5.49 mmol) in DCM (27.5 mL) at rt was added manganese (IV) oxide activated (2.65 g, 27.5 mmol). The mixture was stirred at rt for 12 h. The crude was filtrated over Celite cake and the filtrate was evaporated to dryness. The resulting solid was purified by flash chromatography (100% Heptanes to 20% EtOAc in heptanes) to afford the title compound. Methyl 3-bromo-5-formylbenzoate (854 mg, 64%) as a white solid. ¹H NMR (400 MHz, CDCl₃) δ 10.01 (s, 1H), 8.44 (d, J=1.3 Hz, 1H), 8.42 (d, J=1.7 Hz, 1H), 8.23-8.16 (m, 1H), 3.97 (s, 3H).

### Step 3

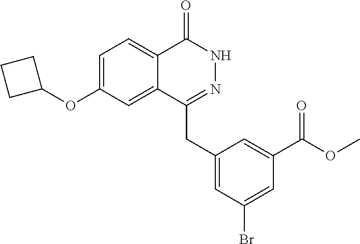 [SMILES: COC(=O)c1cc(Br)cc(Cc2n[nH]c(=O)c3ccc(OC4CCC4)cc23)c1]

### Methyl 3-bromo-5-((7-cyclobutoxy-4-oxo-3,4-dihydrophthalazin-1-yl)methyl)benzoate

A solution of methyl 3-bromo-5-formylbenzoate (625 mg, 2.06 mmol), dimethyl (6-cyclobutoxy-3-oxo-1,3-dihydroisobenzofuran-1-yl)phosphonate (355 mg, 1.14 mmol) and Triethylamine (865 uL, 6.17 mmol) in anhydrous THF (10.3 mL) was stirred at 25° C. under N₂ (g) for 15 hours. Dilution with EtOAc (60 mL) and transfer to a 125 mL extraction funnel. Wash the solution with NaHCO₃ (sat, aq.) and brine (1×). Dry organic layer over Na₂SO₄, filter and evaporate to dryness. The oil was redissolved in anhydrous THF (10.3 mL) and then hydrazine hydrate solution (196 uL, 6.17 mmol) was added and the reaction was stirred at 70° C. for 1 hour. The reaction was cooled to rt, then THF was removed under vacuum. The crude was resuspended in a 75% EtOAc in heptane mix and triturated for 1 hour. Filtration of the solid provided a white solid which was dried for 30 minutes. Methyl 3-bromo-5-((7-cyclobutoxy-4-oxo-3,4-dihydrophthalazin-1-yl)methyl)benzoate (740 mg, 81%) recuperated. LCMS ESI m/z: 444.2 [M+H]⁺.

### Step 4

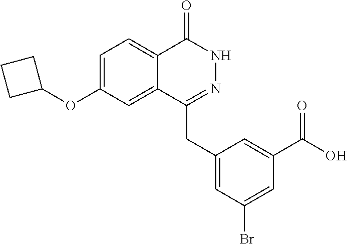 [SMILES: O=C(O)c1cc(Br)cc(Cc2n[nH]c(=O)c3ccc(OC4CCC4)cc23)c1]

### 3-Bromo-5-((7-cyclobutoxy-4-oxo-3,4-dihydrophthalazin-1-yl)methyl)benzoic acid

A solution of methyl 3-bromo-5-((7-cyclobutoxy-4-oxo-3,4-dihydrophthalazin-1-yl)methyl)benzoate (450 mg, 1.02 mmol), 1 M LiOH in H₂O (2.03 mL, 2.03 mmol) and THF (5.08 mL) was stirred at 25° C. under N₂ (g) for 15 hours. The reaction was stopped and the product was precipitated out of solution by addition of 10% citric acid in water until pH was found to be acidic (pH=2-3). Product precipitated as a bright yellow solid. Filtration over a Buchner and the solid was washed with water (2×20 mL), dried in vacuo. 3-Bromo-5-((7-cyclobutoxy-4-oxo-3,4-dihydrophthalazin-1-yl)methyl)benzoic acid used as is for the next step (410 mg, 94%). LCMS ESI m/z 429.2 (M+H)⁺. ¹H NMR (400 MHz, DMSO-d₆) δ 13.31 (s, 1H), 12.44 (s, 1H), 8.13 (d, J=8.8 Hz, 1H), 7.86 (dd, J=5.5, 1.5 Hz, 3H), 7.28 (dd, J=8.8, 2.4 Hz, 1H), 7.11 (d, J=2.3 Hz, 1H), 4.85 (p, J=7.0 Hz, 1H), 4.35 (s, 2H), 2.41-2.26 (m, 2H), 2.07-1.88 (m, 2H), 1.88-1.49 (m, 2H).

### Step 5

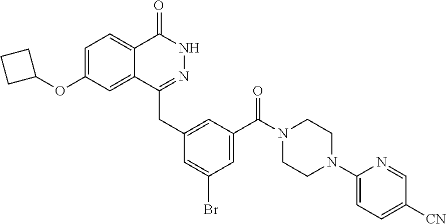 [SMILES: N#Cc1ccc(N2CCN(C(=O)c3cc(Br)cc(Cc4n[nH]c(=O)c5ccc(OC6CCC6)cc45)c3)CC2)nc1]

### 6-(4-(3-Bromo-5-((7-cyclobutoxy-4-oxo-3,4-dihydrophthalazin-1-yl)methyl)benzoyl)piperazin-1-yl)nicotinonitrile

A solution of 3-bromo-5-((7-cyclobutoxy-4-oxo-3,4-dihydrophthalazin-1-yl)methyl)benzoic acid (36.7 mg, 85.4 umol), 6-(piperazin-1-yl)nicotinonitrile (16.1 mg, 85.4 umol) and HATU (40.2 mg, 102 umol) in anhydrous DMF (854 uL) was stirred at 25° C. under N₂ (g) for 2 hours. The crude solution was purified by reverse phase chromatography (100% aqueous ammonium formate (AMF) solution (pH=3.8) to 90% MeCN in AMF). Fractions containing pure product were lyophilized overnight providing 6-(4-(3-bromo-5-((7-cyclobutoxy-4-oxo-3,4-dihydrophthalazin-1-yl)methyl)benzoyl)piperazin-1-yl)nicotinonitrile as a white solid (51.2 mg, 71% yield). LCMS ESI m/z 599.1 (M+H)⁺. 1H NMR (40) MHz, DMSO-d₆) δ 12.45 (s, 1H), 8.49 (d, J=2.5 Hz, 1H), 8.13 (d, J=8.8 Hz, 1H), 7.88 (dd, J=9.0, 2.4 Hz, 1H), 7.68 (s, 1H), 7.51-7.45 (m, 1H), 7.34 (s, 1H), 7.30 (dd, J=8.8, 2.3 Hz, 1H), 7.10 (d, J=2.4 Hz, 1H), 6.89 (d, J=9.0 Hz, 1H), 4.87-4.76 (m, 1H), 4.32 (s, 2H), 3.80-3.51 (m, 6H), 2.35-2.26 (m, 2H), 2.05-1.94 (m, 2H), 1.81-1.54 (m, 2H).

### Synthesis of Example 179: 6-(4-(4-((7-Cyclobutoxy-4-oxo-3,4-dihydrophthalazin-1-yl)methyl)picolinoyl)piperazin-1-yl)nicotinonitrile

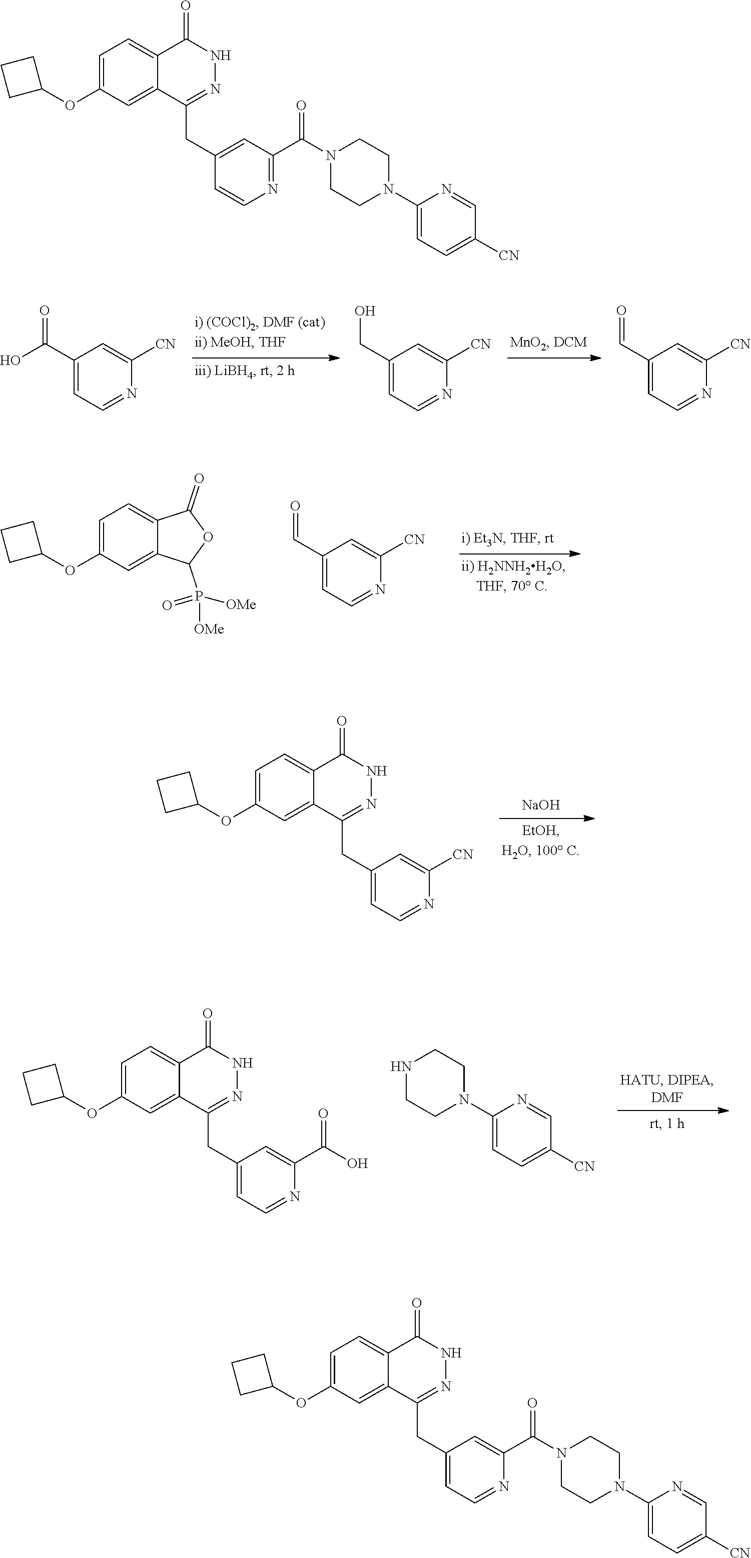

### Step 1

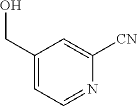

### 4-(Hydroxymethyl)picolinonitrile

To a flame-dried, nitrogen-flushed 100 mL round-bottom flask was added 2-cyanoisonicotinic acid (1.00 g, 6.75 mmol) and anhydrous DCM (10.0 mL). The solution was cooled to 0° C. in a water/ice bath. DMF (2-3 drops) was added along with oxalyl chloride (1.75 mL, 20.3 mmol) dropwise. The reaction was stirred at 0° C. to rt over 2 hours. Then, the solution was evaporated to dryness to provide an orange oil. It was directly redissolved in THF C10.0 mL) and cooled to 0° C. Methanol (5.00 mL) was added and 1 M Lithium borohydride solution in THF (3.38 mL, 6.75 mmol) was added dropwise. The reaction was stirred from 0° C. to rt overnight. Additional amount of 1 M lithium borohydride (3.38 mL, 6.75 mmol) was added dropwise at rt. After 2 more hours, the solution was evaporated to dryness. It resulted in a gum/solid which was redissolved in EtOAc (60 mL). The organic layer was washed with HCl 1N (2×50 mL), and brine (2×50 mL). The organic layer was dried over Na₂SO₄, filtered and evaporated to dryness. It resulted in a yellow solid. Purification of the crude over flash chromatography (100% heptanes to 80% EtOAc in heptanes). Evaporation of the fraction provided 4-(hydroxymethyl)picolinonitrile (450 mg, 50%) as a white solid. LCMS ESI m/z 135.1 (M+H)⁺. ¹H NMR (400 MHz, CDCl₃) δ 8.67 (d, J=5.1 Hz, 1H), 7.73 (s, 1H), 7.51 (d, J=4.5 Hz, 1H), 4.82 (s, 2H), 1.75 (s, 1H).

### Step 2

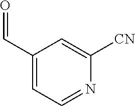

### 4-Formylpicolinonitrile

To a 10) mL round-bottom flask was added 4-(hydroxymethyl)picolinonitrile (450 mg, 3.35 mmol), and it was dissolved in DCM (16.8 mL). The reaction was initiated by addition of Manganese (IV) oxide activated (1.94 g, 20.1 mmol) which created a black suspension. The suspension was vigorously stirred at rt and monitored after 24 hours. The reaction was stopped and filtered over Celite. The celite cake was washed with DCM (2×30 mL) and filtrate was evaporated to dryness. It resulted in a white foam. The crude was then purified by flash chromatography (100% Heptanes to 60% EtOAc in heptanes). Evaporation of clean fraction provided 4-formylpicolinonitrile (110 mg, 25% yield) as a white solid. ¹H NMR (400 MHz, CDCl₃) δ 10.12 (s, 1H), 9.02 (d, J=4.9 Hz, 1H), 8.11 (s, 1H), 7.94 (dd, J=4.9, 1.5 Hz, 1H).

### Step 3

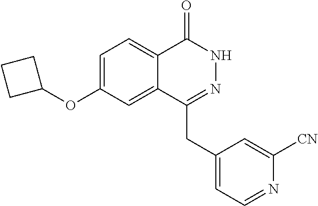 [SMILES: N#Cc1cc(Cc2n[nH]c(=O)c3ccc(OC4CCC4)cc23)ccn1]

### 4-((7-Cyclobutoxy-4-oxo-3,4-dihydrophthalazin-1-yl)methyl)picolinonitrile

A solution of dimethyl (6-cyclobutoxy-3-oxo-1,3-dihydroisobenzofuran-1-yl)phosphonate (208 mg, 0.667 mmol), 4-formylpicolinonitrile (80 mg, 0.606 mmol) and Et₃N (285 mg, 1.82 mmol) in anhydrous THE (6.1 mL) was stirred at 25° C. under Ar (g) for 15 hours. Hydrazine hydrate (38 mg, 1.21 mmol) was added and the reaction was stirred at 70° C. for 2 hours. The reaction was cooled to rt, then THE was removed under vacuum. The formed solids were washed with water (10 mL) and Heptanes/EtOAc (2:1, 30 mL) to give 44(7-cyclobutoxy-4-oxo-3,4-dihydrophthalazin-1-yl)methyl)picolinonitrile (201 mg, 99% yield) as a as a white solid. LCMS ESI m/z: 319.2 [M+H]⁺.

### Step 4

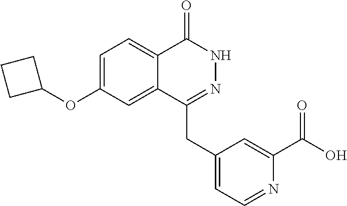 [SMILES: O=C(O)c1cc(Cc2n[nH]c(=O)c3ccc(OC4CCC4)cc23)ccn1]

### 4-((7-Cyclobutoxy-4-oxo-3,4-dihydrophthalazin-1-yl)methyl)picolinic acid

A solution of 4-((7-cyclobutoxy-4-oxo-3,4-dihydrophthalazin-1-yl)methyl)picolinonitrile (201 mg, 605 umol), an 8.0 M aqueous solution of Sodium hydroxide (3.03 mL), and Ethanol (3.02 mL) was stirred at 25° C. then heated to 100° C. for 1 hour. The reaction was cooled to rt, then the reaction was made acidic by addition of citric acid 10% in water (30 mL) until pH was found to be 2-3. The formed solid was triturated and filtered and dried in vacuo. The solid was then triturated again in in 40% EtOAc in 60% Heptanes (30 mL) for 30 minutes before being filtered on a Buchner. 4-((7-Cyclobutoxy-4-oxo-3,4-dihydrophthalazin-1-yl)methyl)picolinic acid (170 mg, 80%) isolated as a white solid. LCMS ESI m/z: 352.2 [M+H]⁺. ¹H NMR (400 MHz, DMSO-d₆) δ 12.48 (s, 1H), 8.56 (d, J=4.9 Hz, 1H), 8.14 (d, J=8.8 Hz, 1H), 8.01 (s, 1H), 7.49 (d, J=4.4 Hz, 1H), 7.30 (dd, J=8.8, 1.9 Hz, 1H), 7.07 (d, J=1.8 Hz, 1H), 4.90-4.74 (m, 1H), 4.39 (s, 2H), 2.48-2.29 (m, 2H), 2.01-1.92 (m, 2H), 1.76 (dd, J=19.0, 9.3 Hz, 1H), 1.62 (dd, J=19.1, 9.4 Hz, 1H).

### Step 5

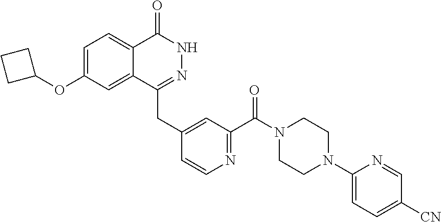 [SMILES: N#Cc1ccc(N2CCN(C(=O)c3cc(Cc4n[nH]c(=O)c5ccc(OC6CCC6)cc45)ccn3)CC2)nc1]

### 6-(4-(4-((7-Cyclobutoxy-4-oxo-3,4-dihydrophthalazin-1-yl)methyl)picolinoyl)piperazin-1-yl)nicotinonitrile

A solution of 4-((7-cyclobutoxy-4-oxo-3,4-dihydrophthalazin-1-yl)methyl)picolinic acid (40.0 mg, 0.114 mmol), 6-(piperazin-1-yl)nicotinonitrile (21.4 mg, 0.114 mmol) and HATU (53.6 mg, 0.137 mmol) in anhydrous DMF (1.14 mL) was stirred at 25° C. under N₂ (g) for 2 hours. The crude solution was purified by reverse phase chromatography (100% aqueous ammonium formate (AMF) solution (pH=3.8) to 60% MeCN in AMF). Fractions containing pure product were lyophilized overnight providing 6-(4-(4-((7-Cyclobutoxy-4-oxo-3,4-dihydrophthalazin-1-yl)methyl)picolinoyl)piperazin-1-yl)nicotinonitrile as a white solid (23.0 mg, 39% yield). LCMS ESI m/z 522.3 (M+H)⁺. ¹H NMR (400 MHz, DMSO-d₆) δ 12.49 (s, 1H), 8.56-8.44 (m, 2H), 8.14 (d, J=8.8 Hz, 1H), 7.87 (dd, J=9.1, 2.4 Hz, 1H), 7.60 (s, 1H), 7.40 (dd, J=5.1, 1.5 Hz, 1H), 7.30 (dd, J=8.8, 2.3 Hz, 1H), 7.06 (d, J=2.3 Hz, 1H), 6.90 (d, J=9.0 Hz, 1H), 4.83 (p, J=6.6 Hz, 1H), 4.37 (s, 2H), 3.82-3.48 (m, 8H), 2.34 (ddd, J=12.7, 9.7, 5.2 Hz, 2H), 2.03-1.90 (m, 2H), 1.82-1.54 (m, 2H).

### Synthesis of Example 180: 6-(4-(2-((7-Cyclobutoxy-4-oxo-3,4-dihydrophthalazin-1-yl)methyl)isonicotinoyl)piperazin-1-yl)nicotinonitrile

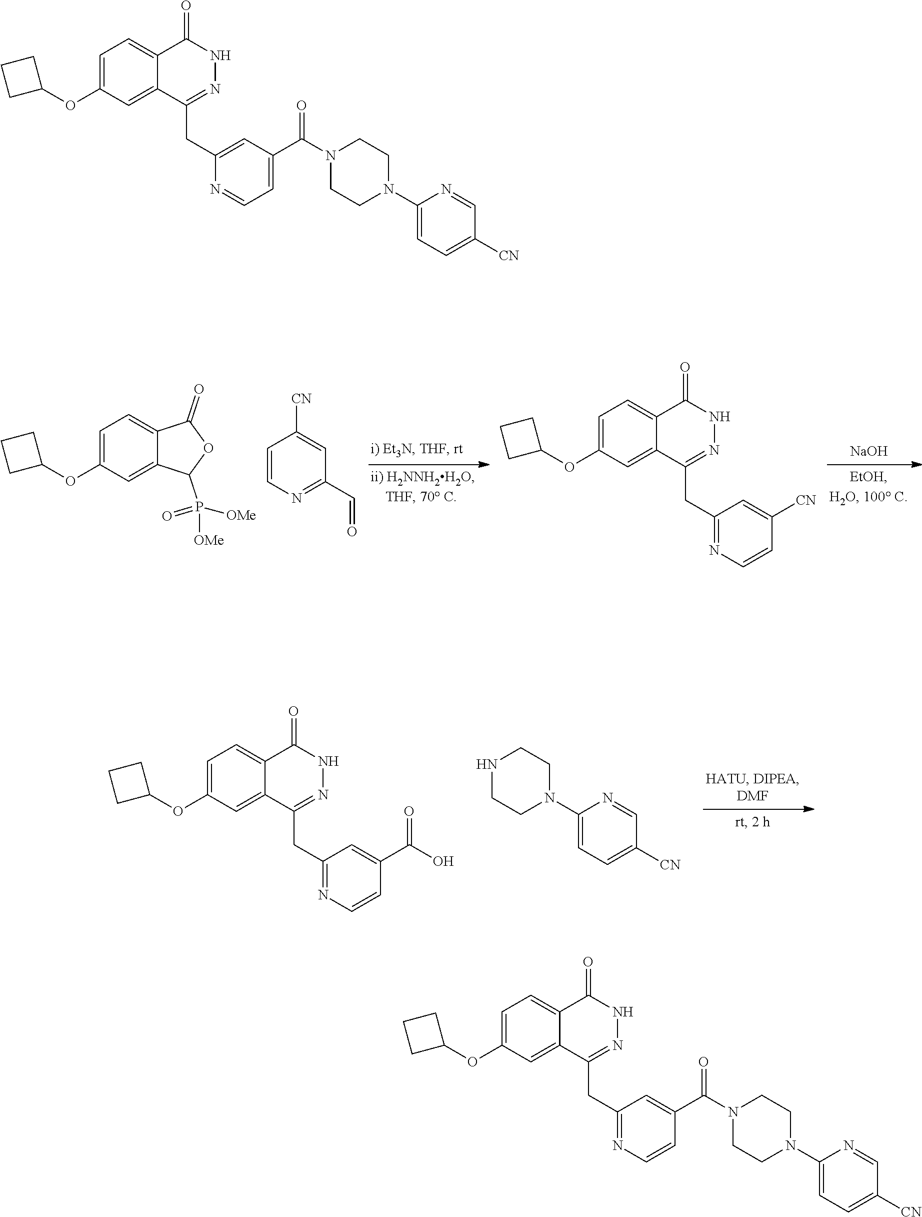

### Step 1

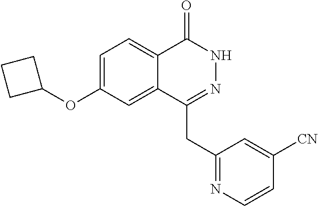 [SMILES: N#Cc1ccnc(Cc2n[nH]c(=O)c3ccc(OC4CCC4)cc23)c1]

### 2-((7-Cyclobutoxy-4-oxo-3,4-dihydrophthalazin-1-yl)methyl)isonicotinonitrile

A solution of dimethyl (6-cyclobutoxy-3-oxo-1,3-dihydroisobenzofuran-1-yl)phosphonate (189 mg, 0.606 mmol), 2-formylisonicotinonitrile (80 mg, 0.606 mmol) and Et₃N (185 mg, 1.82 mmol) in anhydrous THF (3.03 mL) was stirred at 25° C. under Ar (g) for 15 hours. Hydrazine hydrate (38.8 mg, 1.21 mmol) was added and the reaction was stirred at 70° C. for 2 hours. The reaction was cooled to rt, then THF was removed under vacuum. The formed solids were washed with water (10 mL) and petroleum ether/EtOAc (2:1, 10 mL) to give 2-((7-cyclobutoxy-4-oxo-3,4-dihydrophthalazin-1-yl)methyl)isonicotinonitrile (192 mg, 95% yield) as a as a white solid. LCMS ESI m/z 333.1 (M+H)⁺.

### Step 2

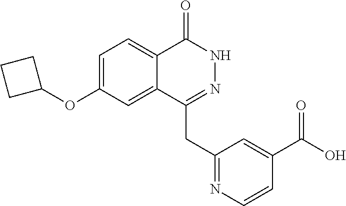 [SMILES: O=C(O)c1ccnc(Cc2n[nH]c(=O)c3ccc(OC4CCC4)cc23)c1]

### 2-((7-Cyclobutoxy-4-oxo-3,4-dihydrophthalazin-1-yl)methyl)isonicotinic acid

A solution of 2-((7-cyclobutoxy-4-oxo-3,4-dihydrophthalazin-1-yl)methyl)isonicotinonitrile (180 mg, 0.542 mmol), an 8.0 M aqueous solution of sodium hydroxide (4.16 mL, 33.3 mmol), and Ethanol (2.71 mL) was stirred at 25° C. then heated to 100° C. for 1 hour. The reaction was cooled to rt, then the reaction was made acidic by addition of citric acid 10% in water (30 mL) until pH was found to be 2-3. The formed solid was triturated and filtered and dried in vacuo. The solid was then triturated again in in 40% EtOAc in 60% Heptanes (30 mL) for 30 minutes before being filtered on a Buchner. 2-((7-cyclobutoxy-4-oxo-3,4-dihydrophthalazin-1-yl)methyl)isonicotinic acid (170 mg, 89% yield) isolated as a white solid. LCMS ESI m/z: 352.3 [M+H]⁺. ¹H NMR (400 MHz, DMSO-d₆) δ 12.43 (s, 1H), 8.58 (d, J=5.0 Hz, 1H), 8.10 (d, J=8.7 Hz, 1H), 7.79 (s, 1H), 7.60 (d, J=3.9 Hz, 1H), 7.25 (dd, J=8.8, 2.3 Hz, 1H), 7.11 (d, J=2.2 Hz 1H), 4.79-4.70 (m, 1H), 4.46 (s, 2H), 2.40-2.27 (m, 2H), 2.04-1.90 (m, 2H), 1.80-1.53 (m, 2H).

### Step 3

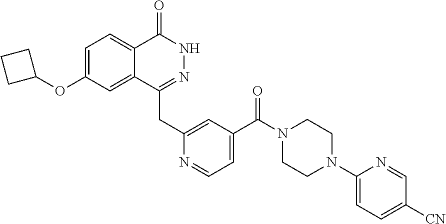 [SMILES: N#Cc1ccc(N2CCN(C(=O)c3ccnc(Cc4n[nH]c(=O)c5ccc(OC6CCC6)cc45)c3)CC2)nc1]

### 6-(4-(2-((7-Cyclobutoxy-4-oxo-3,4-dihydrophthalazin-1-yl)methyl)isonicotinoyl)piperazin-1-yl)nicotinonitrile

A solution of 2-((7-cyclobutoxy-4-oxo-3,4-dihydrophthalazin-1-yl)methyl)isonicotinic acid (40.0 mg, 0.114 mmol), 6-(piperazin-1-yl)nicotinonitrile (21.4 mg, 0.114 mmol) and HATU (53.6 mg, 137 mmol) in anhydrous DMF (1.14 mL) was stirred at 25° C. under N₂ (g) for 2 hours. The crude solution was purified by reverse phase chromatography (100% aqueous ammonium formate (AMF) solution (pH=3.8) to 70% MeCN in AMF). Fractions containing pure product were lyophilized overnight providing 6-(4-(2-((7-cyclobutoxy-4-oxo-3,4-dihydrophthalazin-1-yl)methyl)isonicotinoyl)piperazin-1-yl)nicotinonitrile as a white solid (18 mg, 30% yield). LCMS ESI m/z 522.3 (M+H)⁺. ¹H NMR (400 MHz, DMSO-d₆) δ 12.45 (s, 1H), 8.50 (d, J=2.2 Hz, 1H), 8.14 (d, J=8.8 Hz, 1H), 7.90 (dd, J=9.4, 6.6 Hz, 2H), 7.52 (d. J=7.7 Hz, 1H), 7.48 (d, J=7.7 Hz, 1H), 7.29 (dd, J=8.8, 2.2 Hz, 1H), 7.08 (d, J=2.3 Hz, 1H), 6.84 (d, J=9.1 Hz, 1H), 4.78-4.67 (m, 1H), 4.46 (s, 2H), 3.72-3.64 (m, 4H), 3.29-3.21 (m, 2H), 2.35-2.27 (m, 2H), 1.99-1.86 (m, 2H), 1.78-1.52 (m, 2H).

### Synthesis of Example 181: 4-(4-(4-Fluoro-3-(4-methylpiperazine-1-carbonyl)benzyl)-1-oxo-1,2-dihydrophthalazin-6-yloxy)benzamide

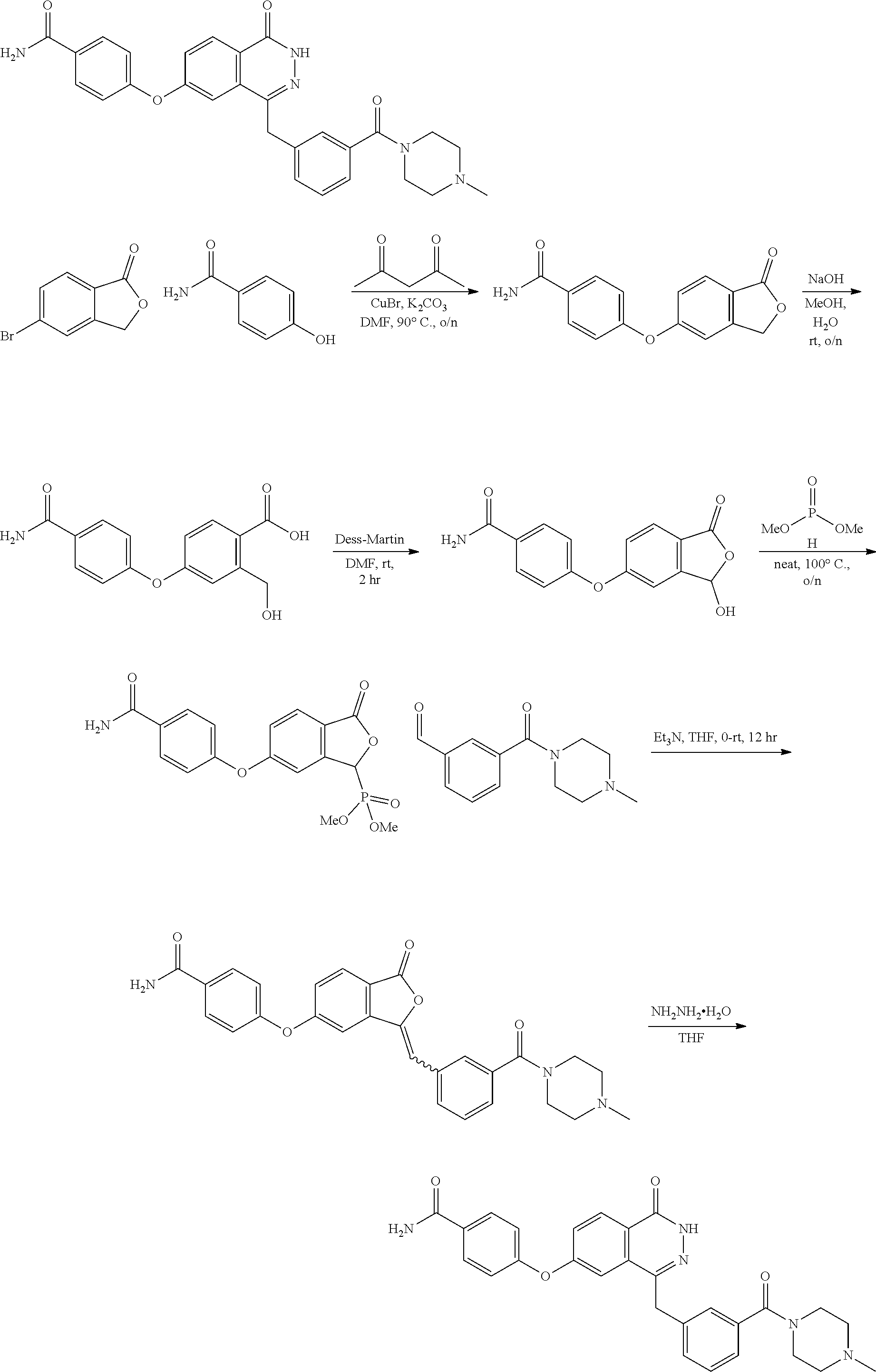

### Step 1

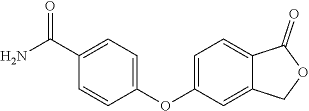 [SMILES: NC(=O)c1ccc(Oc2ccc3c(c2)COC3=O)cc1]

### 4-(1-oxo-1,3-dihydroisobenzofuran-5-yloxy)benzamide

To a mixture of 5-bromoisobenzofuran-1(3H)-one (5.0 g, 24 mmol) and 4-hydroxybenzamide (4.5 g, 33 mmoL) in dry DMF (100 mL) was successively added CuBr (0.65 g, 4.5 mmol), pentane-2,4-dione (0.45 g, 4.5 mmol) and K₂CO₃ (4.5 g, 33 mmol) at room temperature. The resulting mixture was flushed with N₂ for 10 min. Then the reaction was allowed to heat to 90° C. stirred at this temperature for overnight. The slurry was filtered over a pad of celite. The filtrate was diluted with H₂O (100 mL). The mixture was extracted with EA (50 mL, 3). The combined organic layers were washed with 1 N NaOH (40 mL×2). The organic layer was separated, dried, filtered and concentrated. The residue was purified by silica gel chromatography (petroleum ether/ethyl acetate=10/1 to 1/1, v/v) to give 4-(1-oxo-1,3-dihydroisobenzofuran-5-yloxy)benzamide (1.7 g, 27% yield) as a light yellow solid. LCMS ESI m/z: 270.0 [M+H]⁺. ¹H NMR (400 MHz, CDCl₃) δ 7.98 (d, J=8.0 Hz, 2H), 7.88-7.86 (m, 1H), 7.74-7.72 (m, 1H), 7.71 (s, 1H), 7.37 (s, 1H), 7.20 (d, J=8.0 Hz, 2H), 6.79-6.75 (m, 1H), 5.34 (s, 2H).

### Step 2

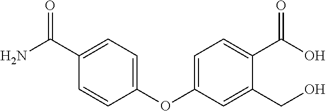 [SMILES: NC(=O)c1ccc(Oc2ccc(C(=O)O)c(CO)c2)cc1]

### 4-(4-Carbamoylphenoxy)-2-(hydroxymethyl)benzoic acid

To a solution of 4-(1-oxo-1,3-dihydroisobenzofuran-5-yloxy)benzamide (1.5 g, 5.6 mmol) in MeOH (15 mL) and H₂O (15 mL) was added NaOH (0.60 g, 17 mmol) at room temperature. The resulting mixture was stirred at the same temperature for overnight. The volatile was removed. The residue was dissolved in H₂O (40 mL). The solution was acidified with 1 N HCl until the solution reached PH 4˜5. The solids were collected by filtration and dried under vacuum to give 4-(4-carbamoylphenoxy)-2-(hydroxymethyl)benzoic acid (1.3 g, 80% yield) as a white solid, which was directly used for the next step without further purification. ¹H NMR (400 MHz. CDCl₃) δ 7.96-7.94 (m, 4H), 7.36 (m, 2H), 7.15-7.12 (m, 2H), 6.99 (m, 1H), 4.84 (d, J=8.0 Hz, 2H).

### Step 3, 4, 5 and 6

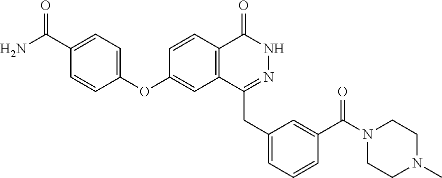 [SMILES: CN1CCN(C(=O)c2cccc(Cc3n[nH]c(=O)c4ccc(Oc5ccc(C(N)=O)cc5)cc34)c2)CC1]

### 4-(4-(4-Fluoro-3-(4-methylpiperazine-1-carbonyl)benzyl)-1-oxo-1,2-dihydrophthalazin-6-yloxy)benzamide

Following the four-step procedure in Example 1, but starting with 4-(4-carbamoylphenoxy)-2-(hydroxymethyl)benzoic acid gave the title compound as a white solid. LCMS ESI m/z: 515.8 [M+H]⁺. ¹H NMR (400 MHz, DMSO-d6) δ 12.58 (s, 1H), 8.28 (d, J=8.0 Hz, 1H), 7.98 (s, 1H), 7.96-7.93 (m, 2H), 7.49 (d, J=4.0 Hz, 1H), 7.45 (dd, J=4.0 Hz, J=8.0 Hz, 1H), 7.37 (s, 1H), 7.32-7.28 (m, 1H), 7.25 (dd, J=4.0 Hz, J=8.0 Hz, 1H), 7.18 (t, J=12.0 Hz, 1H), 7.12-7.10 (m, 2H), 4.24 (s, 2H), 3.60 (m, 2H), 3.11 (m, 2H), 2.33-2.30 (m, 2H), 2.14 (m, 5H).

### Synthesis of Example 182: 6-(4-(3-((7-hydroxy-4-oxo-3,4-dihydrophthalazin-1-yl)methyl)benzoyl)piperazin-1-yl)nicotinonitrile

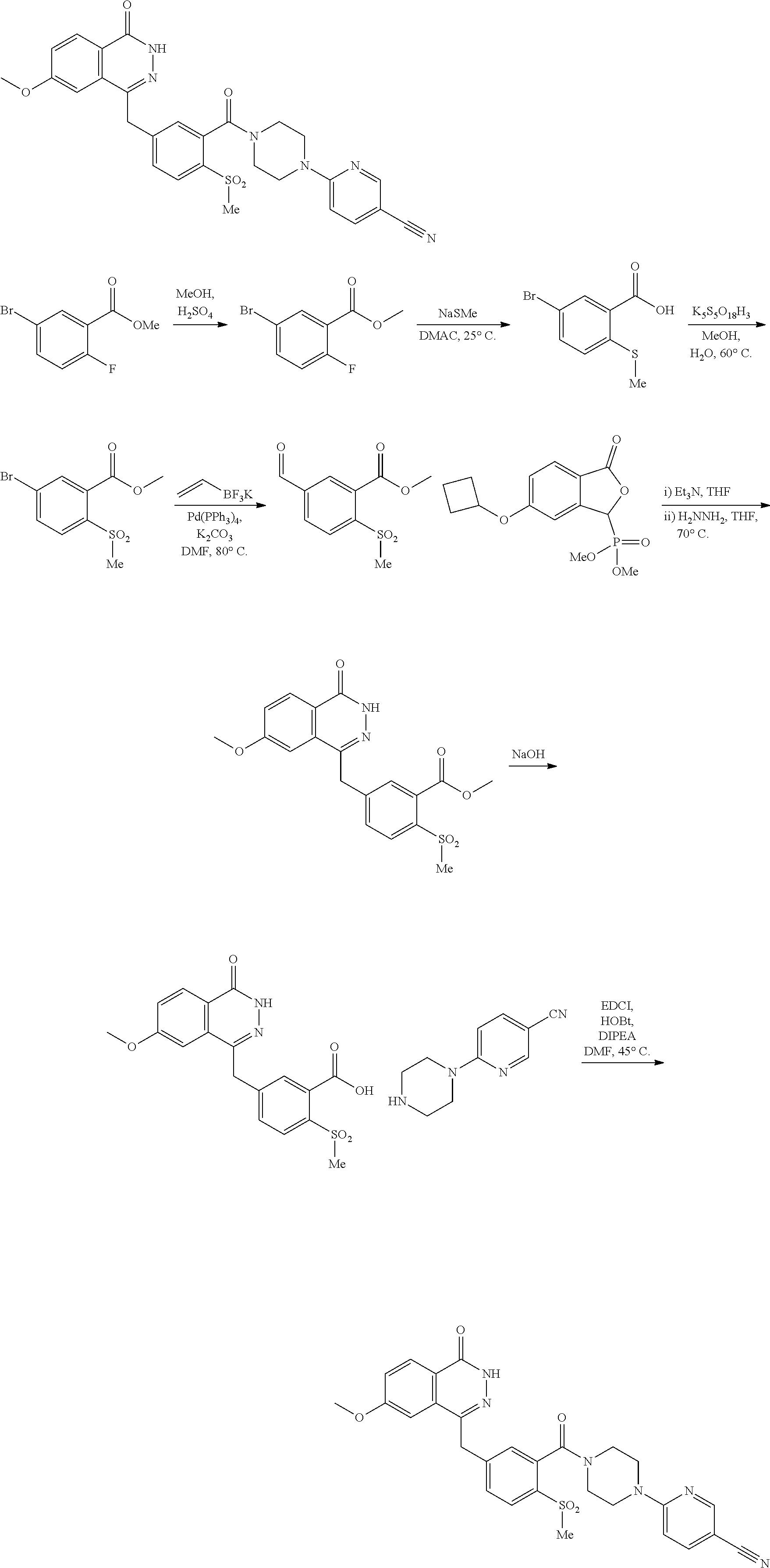

### Step 1

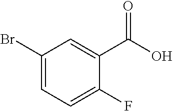 [SMILES: O=C(O)c1cc(Br)ccc1F]

### Methyl 5-bromo-2-fluorobenzoate

To a solution of 5-bromo-2-fluorobenzoic acid (2.0 g, 9.13 mmol) in MeOH (5 mL) was added conc. H₂SO₄ (2 mL) in portions. The mixture was stirred at 25° C. for 20 hours. The solution was concentrated in vacuo. This residue was diluted with water (30 mL) and extracted with EtOAc (20 mL×3). The combined organic layer was washed with brine, dried over anhydrous Na₂SO₄, filtered and concentrated in vacuo to give methyl methyl 5-bromo-2-fluorobenzoate (2.03 g, 95% yield) as a colorless oil. LCMS ESI m/z: 233 [M+H]⁺.

### Step 2

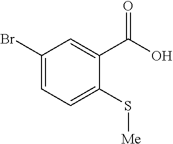 [SMILES: CSc1ccc(Br)cc1C(=O)O]

### Methyl 5-bromo-2-(methylthio)benzoate

To a solution of methyl 5-bromo-2-fluorobenzoate (2.03 g, 8.71 mmol) in DMAC (20 mL) was added sodium thiomethoxide (0.69 g, 9.58 mmol) in portions. The mixture was stirred at 50° C. for 3 hours, then diluted with water (30 mL) and extracted with EtOAc (20 mL×3). The combined organic layer was washed with brine, dried over anhydrous Na₂SO₄, filtered and concentrated in vacuo to give methyl 5-bromo-2-(methylthio)benzoate (1.6 g, 70.34% yield) as a colorless oil. LCMS ESI m/z: 261/263 [M+H]⁺. This was used without further purification.

### Step 3

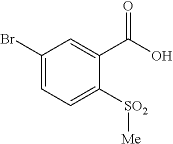 [SMILES: CS(=O)(=O)c1ccc(Br)cc1C(=O)O]

### Methyl 5-bromo-2-(methylsulfonyl)benzoate

To a solution of methyl 5-bromo-2-(methylthio)benzoate (1.6 g, 6.13 mmol) in MeOH (20 mL), was added a solution of potassium peroxymonosulfate (0.69 g, 9.58 mmol) in H₂O (5 mL) dropwise. The mixture was stirred at 60° C. for 2 hours. The solution was concentrated in vacuo. This residue was diluted with water (30 mL) and extracted with EtOAc (20 mL×3). The combined organic layer was washed with brine, dried over anhydrous Na₂SO₄, filtered and concentrated in vacuo to give methyl 5-bromo-2-(methylsulfonyl)benzoate (1.55 g, 86% yield) as a white solid. LCMS ESI m/z: 293/295 [M+H]⁺. This was used without further purification.

### Step 4

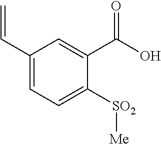

### Methyl 2,4-difluoro-5-vinylbenzoate

A mixture of bromo-2-methylsulfonyl)benzoate (560 mg, 1.91 mmol), potassium vinyltrifluoroborate (264 mg, 1.97 mmol). K₂CO₃ (660 mg, 4.78 mmol) and Pd(PPh₃)₄ (110 mg, 0.95 mmol) in DMF (10 mL) was stirred at 80° C. under Ar (g) for 4 hours. The mixture was cooled to rt, diluted with H₂O (5 mL) and extracted with EtOAc (10 mL×3). The combined organic layer was washed with brine, dried over anhydrous Na₂SO₄, filtered and concentrated in vacuo. The residue was purified by silica gel chromatography (EtOAc/petroleum ether=0% to 7%) to give methyl 2-(methylsulfonyl)-5-vinylbenzoate (453 mg, 98% yield) as a colorless oil. LCMS ESI m/z: 241 [M+H]⁺.

### Step 5

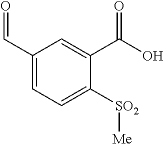 [SMILES: CS(=O)(=O)c1ccc(C=O)cc1C(=O)O]

### 5-Formyl-2-(methylsulfonyl)benzoic acid

To a mixture of methyl 2,4-difluoro-5-vinyl-benzoate (453 mg, 1.89 mmol) in THF (24 mL) and H₂O (18 mL) was added K₂OsO₄-2H₂O (7 mg, 0.02 mmol). The mixture was stirred at room temperature for 5 min. then NaIO₄ (1.28 g, 6 mmol) was added and the mixture was stirred at 80° C. for 2 hours. The solids were removed by filtration. The filtrate was diluted with H₂O (5 mL) and extracted with EtOAc (30 mL×3). The combined organic layer was washed with brine, dried over anhydrous Na2SO4, filtered and concentrated in vacuo to give methyl 5-formyl-2-(methylsulfonyl)benzoate (210 mg, 43% yield) as a colorless oil. LCMS ESI m/z: 243 [M+H]⁺. This was used without further purification.

### Step 6

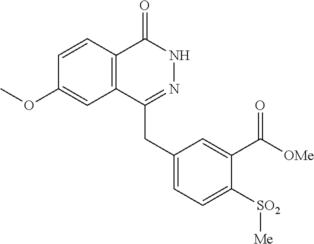 [SMILES: COC(=O)c1cc(Cc2n[nH]c(=O)c3ccc(OC)cc23)ccc1S(C)(=O)=O]

### Methyl 5-((7-methoxy-4-oxo-3,4-dihydrophthalazin-1-yl)methyl)-2-(methylsulfonyl) benzoate

A solution of dimethyl (6-methoxy-3-oxo-1,3-dihydroisobenzofuran-1-yl)phosphonate (211 mg, 0.78 mmol), methyl 5-formyl-2-(methylsulfonyl)benzoate (237 mg, 0.78 mmol) and Et₃N (238 mg, 2.35 mmol) in anhydrous THF (16 mL) was stirred at 25° C. under Ar (g) for 3 hours. Hydrazine hydrate (39 mg, 1.17 mmol) was added and the reaction was stirred at 70° C. for 2 hour. The reaction was cooled to rt, then THF was removed under vacuum. The formed solids were washed with water (10 mL) and petroleum ether/EtOAc (10:1, 10 mL) to give methyl 5-((7-methoxy-4-oxo-3,4-dihydrophthalazin-1-yl)methyl)-2-(methylsulfonyl)benzoate (154 mg, 48% yield) as a yellow solid. LCMS ESI m/z: 403 [M+H]⁺.

### Step 7

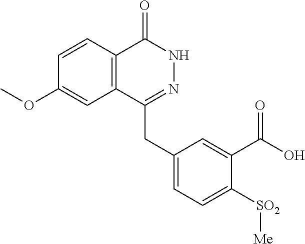 [SMILES: COc1ccc2c(=O)[nH]nc(Cc3ccc(S(C)(=O)=O)c(C(=O)O)c3)c2c1]

### 5-((7-Methoxy-4-oxo-3,4-dihydrophthalazin-1-yl)methyl)-2-(methylsulfonyl)benzoic acid

A solution of methyl 5-((7-methoxy-4-oxo-3,4-dihydrophthalazin-1-yl)methyl)-2-(methylsulfonyl)benzoate (97 mg, 0.26 mmol) and NaOH (32 mg, 0.79 mmol) in MeOH (2 mL), THF (2 mL) and H₂O (2 mL) was stirred at rt for 1 hour. The organic solvent was removed in vacuum and aq HCl (1N) was added until the solution reached to pH 5˜6. The solids were collected by filtration, washed with water and dried under vacuum to give 5-((7-methoxy-4-oxo-3,4-dihydrophthalazin-1-yl)methyl)-2-(methylsulfonyl) benzoic acid (102 mg, 68% yield) as a white solid. LCMS ESI m/z: 389 [M+H]⁺. This was used without further purification.

### Step 8

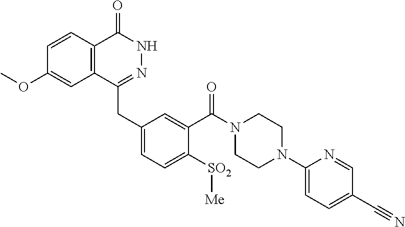 [SMILES: COc1ccc2c(=O)[nH]nc(Cc3ccc(S(C)(=O)=O)c(C(=O)N4CCN(c5ccc(C#N)cn5)CC4)c3)c2c1]

### 6-(4-(5-((7-Methoxy-4-oxo-3,4-dihydrophthalazin-1-yl)methyl)-2-(methylsulfonyl) benzoyl)piperazin-1-yl)nicotinonitrile

To a solution of 6-piperazin-1-ylpyridine-3-carbonitrile (130 mg, 0.57 mmol) and 5-((7-methoxy-4-oxo-3,4-dihydrophthalazin-1-yl)methyl)-2-(methylsulfonyl)benzoic acid (102 mg, 0.26 mmol) in DMF (4 mL) was added EDCI (75 mg, 0.39 mmol). HOBt (53 mg, 0.39 mmol) and DIPEA (102 mg, 0.79 mmol). The reaction mixture was stirred 45° C. for 1 hour. The reaction was purified by prep-HPLC to give the 6-(4-(5-((7-methoxy-4-oxo-3,4-dihydrophthalazin-1-yl)methyl)-2-(methylsulfonyl)benzoyl)piperazin-1-yl) nicotinonitrile (55 mg, 37% yield) as a white solid. LCMS ESI m/z: 559 [M+H]⁺. ¹H-NMR (400 MHz, DMSO-D6) δ 12.46 (s, 1H), 8.52 (d, J=2.1 Hz, 1H), 8.19 (d, J=8.8 Hz, 1H), 7.95-7.88 (m, 2H), 7.63 (dd, J=8.2, 1.5 Hz, 1H), 7.57 (d, J=1.3 Hz, 1H), 7.43 (dd, J=8.8, 2.4 Hz, 1H), 7.33 (d, J=2.3 Hz, 1H), 6.92 (d, J=9.1 Hz, 1H), 4.46 (d, J=2.4 Hz, 2H), 3.90 (s, 3H), 3.86-3.59 (m, 6H), 3.27 (s, 3H), 3.25-3.12 (m, 2H).

### Synthesis of Example 183: 6-(4-(2-Fluoro-5-((4-oxo-7-(2,2,2-trifluoroethyl)-3,4-dihydrophthalazin-1-yl)methyl)benzoyl)piperazin-1-yl)nicotinonitrile

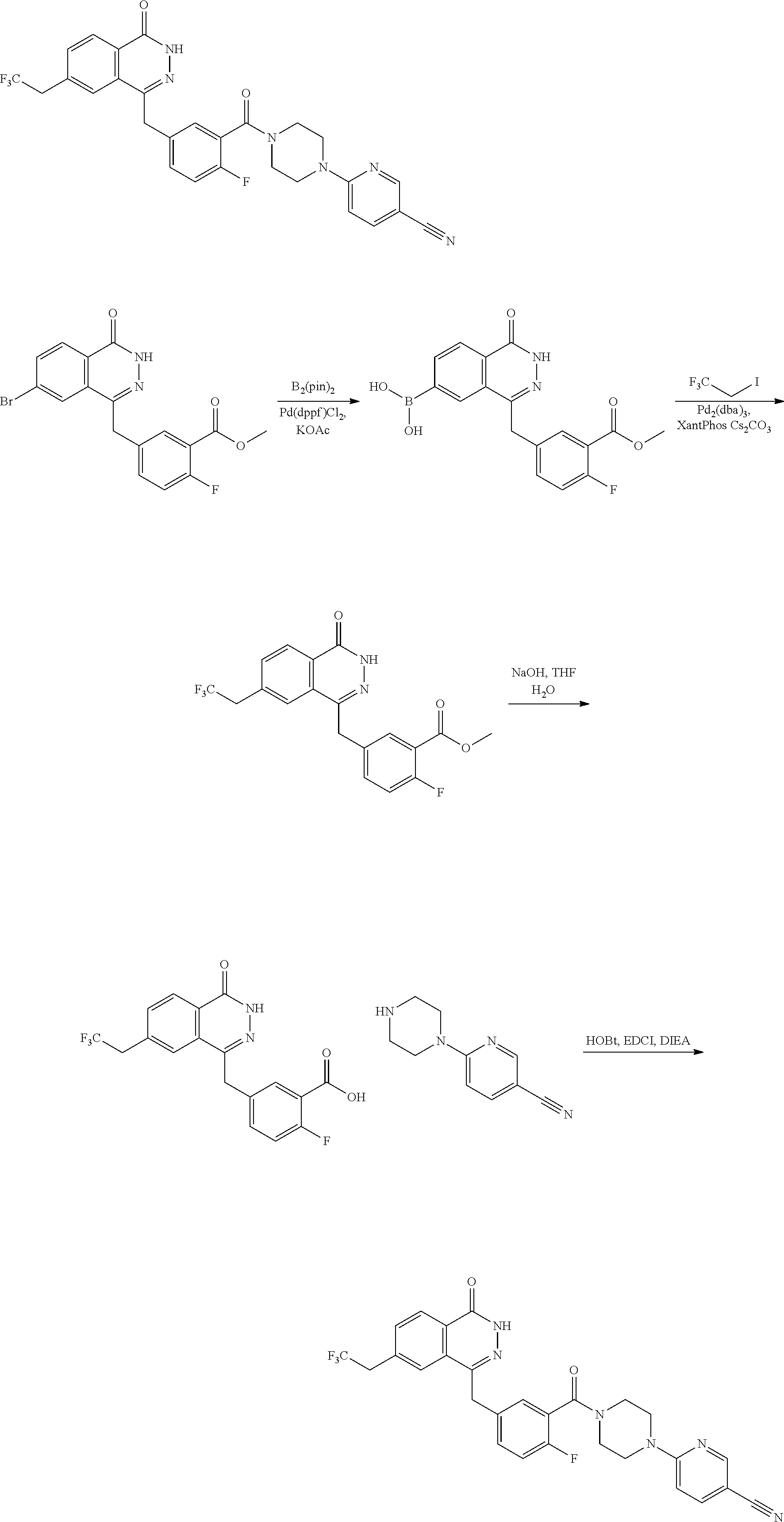

### Step 1

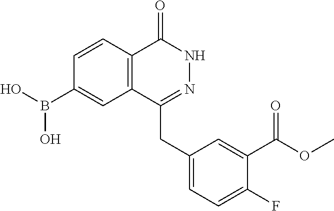 [SMILES: COC(=O)c1cc(Cc2n[nH]c(=O)c3ccc(B(O)O)cc23)ccc1F]

### (4-(4-Fluoro-3-(methoxycarbonyl)benzyl)-1-oxo-1,2-dihydrophthalazin-6-yl)boronic acid

A mixture of methyl 5-(7-bromo-4-oxo-3,4-dihydrophthalazin-1-yl)methyl)-2-fluorobenzoate (529 mg, 1.11 mmol), 4,4,4′,4′,5,5,5′,5′-octamethyl-2,2′-bi(1,3,2-dioxaborolane) (1.41 g, 5.54 mmol), KOAc (653 mg, 6.65 mmol) and Pd(dppf)Cl₂ (49 mg, 0.06 mmol) in DMSO (10 mL) was stirred at 80° C. under Ar (g) for 2 hours. The mixture was cooled to rt, diluted with H₂O (10 mL) and extracted with EtOAc (20 mL×3). The combined organic layer was washed with brine, dried over anhydrous Na₂SO₄, filtered and concentrated in vacuo to afford (4-(4-fluoro-3-(methoxycarbonyl)benzyl)-1-oxo-1,2-dihydrophthalazin-6-yl)boronic acid as a light yellow solid (520 mg, 67% yield). This was used without further purification. LCMS ESI 357 (M+H)⁺.

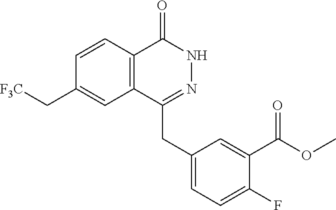 [SMILES: COC(=O)c1cc(Cc2n[nH]c(=O)c3ccc(CC(F)(F)F)cc23)ccc1F]

### tert-Butyl 4-(5-cyanopyridin-2-yl)-4-fluoropiperidine-1-carboxylate

To a mixture of (4-(4-fluoro-3-(methoxycarbonyl)benzyl)-1-oxo-1,2-dihydrophthalazin-6-yl)boronic acid (520 mg, 0.74 mmol), 1,1,1-trifluoro-2-iodoethane (781 mg, 3.72 mmol) and Cs₂CO₃ (970 mg, 2.98 mmol) in DMSO (10 mL) was added Pd₂(dba)₃ (41 mg, 0.05 mmol) and XantPhos (73 mg, 0.13 mmol) at r under Ar (g). The reaction was stirred at 65° C. for 16 hours. The mixture was cooled to rt, diluted with H₂O (10 mL) and extracted with EtOAc (20 mL×3). The combined organic layer was concentrated in vacuo and the residue was purified by prep-HPLC to give dimethyl (6-(difluoromethoxy)-3-oxo-1,3-dihydroisobenzofuran-1-yl)phosphonate as a light yellow oil (60 mg, 20% yield). LCMS ESI 357 (M+H)⁺.

### Step 3

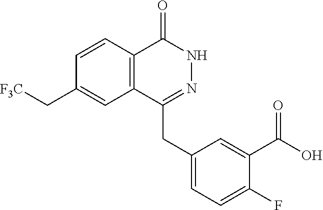 [SMILES: O=C(O)c1cc(Cc2n[nH]c(=O)c3ccc(CC(F)(F)F)cc23)ccc1F]

### 2-Fluoro-5-((4-oxo-7-(2,2,2-trifluoroethyl)-3,4-dihydrophthalazin-1-yl)methyl)benzoic acid

A mixture of tert-butyl 4-(5-cyanopyridin-2-yl)-4-fluoropiperidine-1-carboxylate (60 mg, 0.15 mmol) and NaOH (18 mg, 0.46 mmol) in THF (3 mL) and H₂O (1 mL) was stirred at rt for 1 hour. The organic solvent was removed under vacuum and aq HCl (1N) was added until the solution reached pH 5˜6. The solids were collected by filtration, washed with water and dried under vacuum to give 2-fluoro-5-((4-oxo-7-(2,2,2-trifluoroethyl)-3,4-dihydrophthalazin-1-yl)methyl)benzoic acid as a white solid (57 mg, 99% yield). This was used without further purification. LCMS ESI 381 (M+H)⁺.

### Step 4

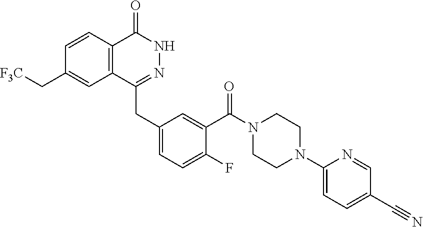 [SMILES: N#Cc1ccc(N2CCN(C(=O)c3cc(Cc4n[nH]c(=O)c5ccc(CC(F)(F)F)cc45)ccc3F)CC2)nc1]

### 6-(4-(2-Fluoro-5-((4-oxo-7-(2,2,2-trifluoroethyl)-3,4-dihydrophthalazin-1-yl)methyl)benzoyl)piperazin-1-yl)nicotinonitrile

To a solution of 2-fluoro-5-((4-oxo-7-(2,2,2-trifluoroethyl)-3,4-dihydrophthalazin-1-yl)methyl)benzoic acid (57 mg, 0.15 mmol) and 6-(piperazin-1-yl)nicotinonitrile (44 mg, 0.18 mmol) in DMF (2 mL) was added EDCI (35 mg, 0.23 mmol). HOBt (31 mg, 0.23 mmol) and DIPEA (98 mg, 0.76 mmol). The reaction mixture was stirred at rt for 14 hours. The reaction was purified by prep-HPLC to give 6-(4-(2-fluoro-5-(4-oxo-7-(2,2,2-trifluoroethyl)-3,4-dihydrophthalazin-1-yl)methyl)benzoyl)piperazin-1-yl)nicotinonitrile as a white solid (28 mg, 32% yield). LCMS ESI 551 (M+H)⁺. ¹H NMR (400 MHz, CDCl₃) δ 10.27 (s, 1H), 8.46 (d, J=8.1 Hz, 1H), 8.42 (d, J=1.8 Hz, 1H), 7.71-7.61 (m, 3H), 7.36 (d, J=5.5 Hz, 2H), 7.07 (t, J=11.2, 6.6 Hz, 1H), 6.61 (d, J=9.0 Hz, 1H), 4.30 (s, 2H), 3.89 (s, 4H), 3.78 (t, 2H), 3.66 (t, J=5.1 Hz, 2H), 3.53 (dd, J=21.5, 11.0 Hz, 2H).

### Synthesis of Example 184: 6-(5-(3-((4-Oxo-7-(pyridin-3-yl)-3,4-dihydrophthalazin-1-yl)methyl)benzoyl)-2,5-diazabicyclo[2.2.1]heptan-2-yl)nicotinonitrile

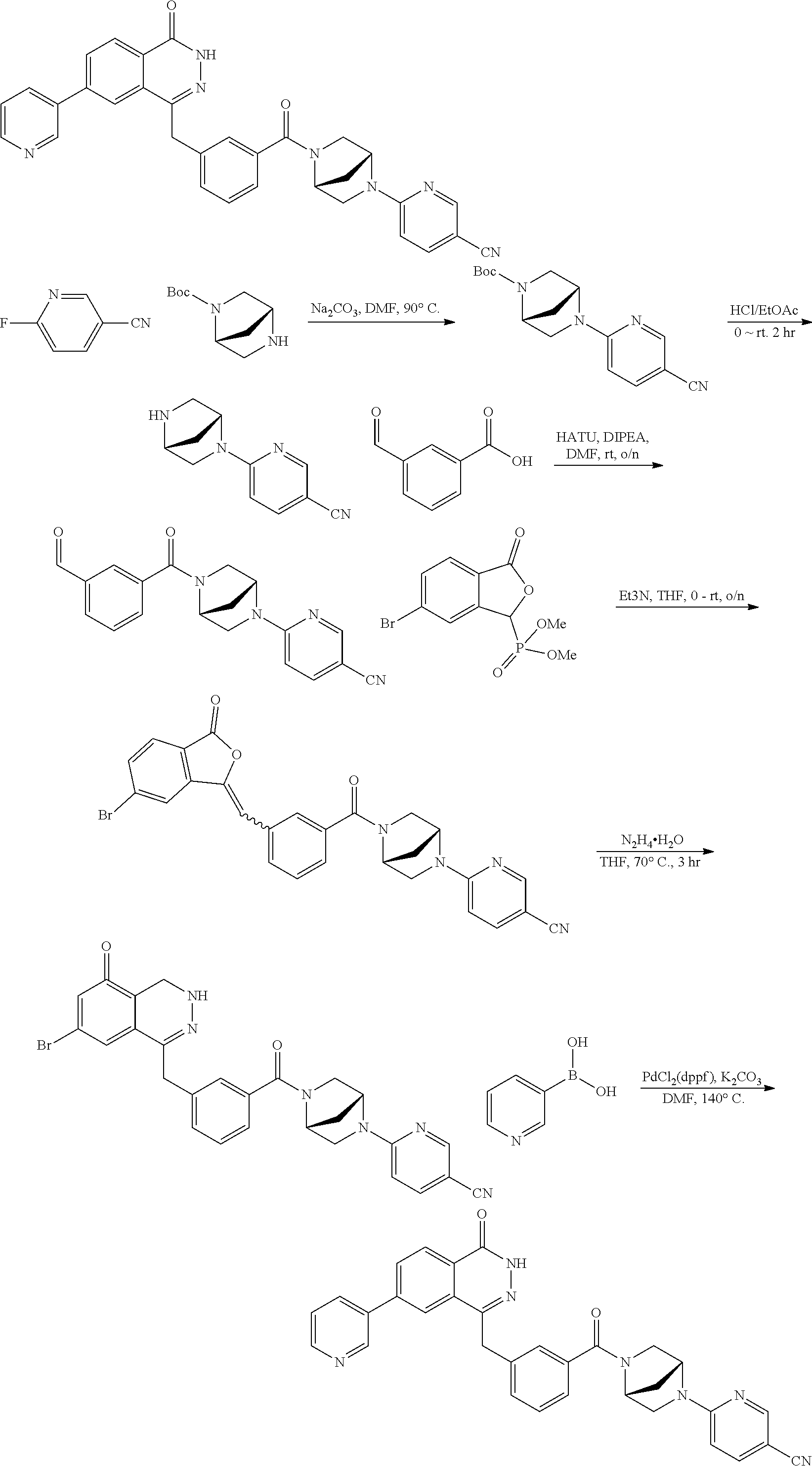

### Step 1, 2, 3, 4 and 5

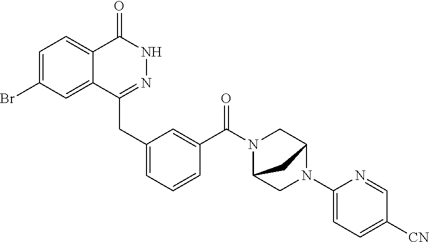 [SMILES: N#Cc1ccc(N2C[C@H]3C[C@@H]2CN3C(=O)c2cccc(Cc3n[nH]c(=O)c4ccc(Br)cc34)c2)nc1]

### 6-(5-(3-((7-Bromo-4-oxo-3,4-dihydrophthalazin-1-yl)methyl)benzoyl)-2,5-diazabicyclo[2.2.1]heptan-2-yl)nicotinonitrile

Following the general procedure in Example 1, but starting with 6-fluoronicotinonitrile and tert-butyl 2,5-diazabicyclo[2.2.1]heptane-2-carboxylate gave the title compound as a yellow solid. LCMS ESI m/z: 541.1 [M+H]⁺.

### Step 6

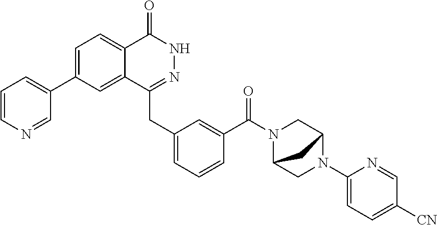 [SMILES: N#Cc1ccc(N2C[C@H]3C[C@@H]2CN3C(=O)c2cccc(Cc3n[nH]c(=O)c4ccc(-c5cccnc5)cc34)c2)nc1]

### 6-(5-(3-((4-Oxo-7-(pyridin-3-yl)-3,4-dihydrophthalazin-1-yl)methyl)benzoyl)-2,5-diazabicyclo[2.2.1]heptan-2-yl)nicotinonitrile

To a solution of 6-(5-(3-((7-bromo-4-oxo-3,4-dihydrophthalazin-1-yl)methyl)benzoyl)-2,5-diazabicyclo[2.2.1]heptan-2-yl)nicotinonitrile (90 mg, 0.16 mmol) and pyridin-3-ylboronic acid (23 mg, 0.18 mmol) in DMF (2 ml) was added Pd(dppf)Cl₂ (12 mg, 0.016 mmol) and K₂CO₃ (69 mg, 0.50 mmol) at 25° C. The reaction mixture was stirred at 140° C. for 4 hr under N₂. The mixture was diluted with water and extracted with EtOAc, the organic layer was dried over anhydrous Na₂SO₄, filtered and concentrated to the crude, which was purified by prep-HPLC to give 6-(5-(3-((4-oxo-7-(pyridin-3-yl)-3,4-dihydrophthalazin-1-yl)methyl)benzoyl)-2,5-diazabicyclo[2.2.1]heptan-2-yl)nicotinonitrile (22 mg, 25% yield) as a white solid. LCMS ESI m/z: 539.7 [M+H]⁺. ¹H NMR (400 MHz, DMSO-d₆) δ 12.66 (d, J=20.4 Hz, 1H), 8.98 (d, J=9.3 Hz, 1H), 8.67 (s, 1H), 8.30 (ddd, J=45.5, 32.0, 23.2 Hz, 5H), 7.80 (dd, J=38.9, 9.0 Hz, 1H), 7.61-7.27 (m, 5H), 5.54-4.67 (m, 2H), 4.58-4.19 (m, 3H), 3.61 (d, J=10.3 Hz, 2H), 3.18-3.03 (m, 1H), 1.98 (dd, J=18.3, 10.3 Hz, 2H).

### Synthesis of Example 185: 4-(4-Fluoro-3-(3-(trifluoromethyl)-5,6,7,8-tetrahydro-[1,2,4]triazolo[4,3-a]pyrazine-7-carbonyl)benzyl)-6-(prop-2-yn-1-ylamino)phthalazin-(2H)-one

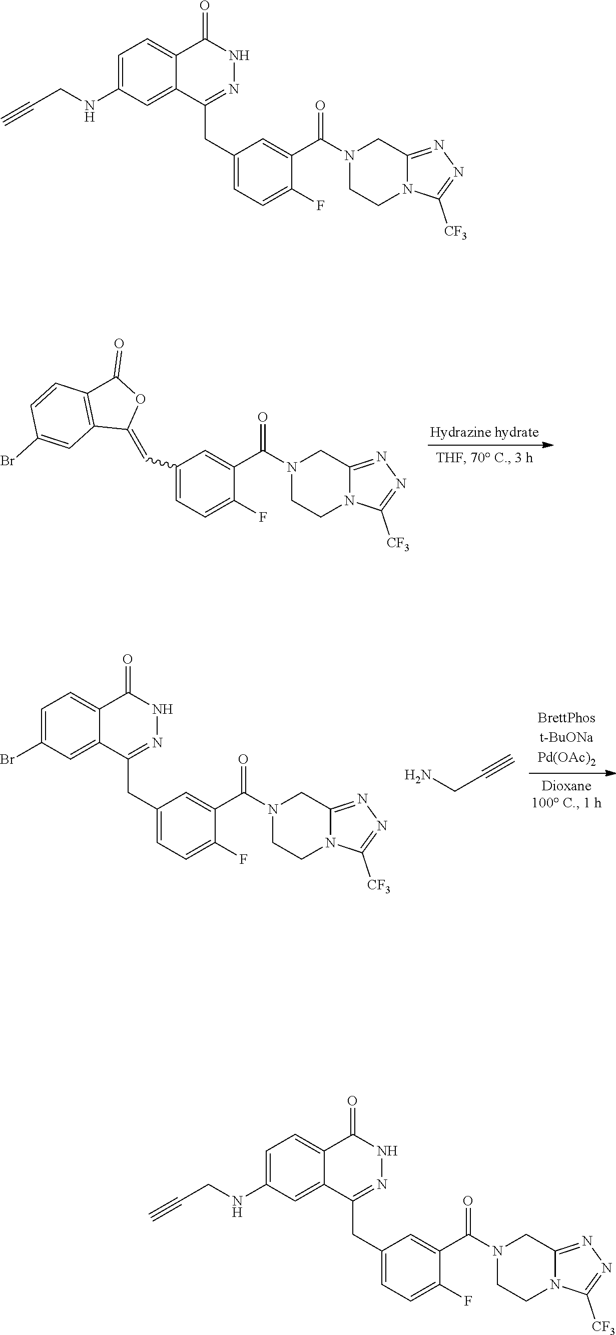

### Step 1

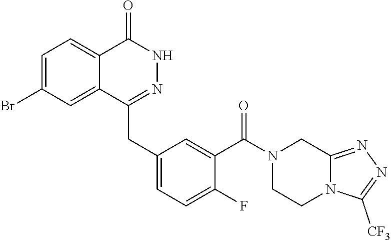 [SMILES: O=C(c1cc(Cc2n[nH]c(=O)c3ccc(Br)cc23)ccc1F)N1CCn2c(nnc2C(F)(F)F)C1]

### 6-Bromo-4-(4-fluoro-3-(3-(trifluoromethyl)-5,6,7,8-tetrahydro-[1,2,4]triazolo[4,3-a]pyrazine-7-carbonyl)benzyl)phthalazin-1(2H)-one

To a solution of 5-bromo-3-[[4-fluoro-3-[3-(trifluoromethyl)-6,8-dihydro-5H-[1,2,4]triazolo[4,3-a]pyrazine-7-carbonyl]phenyl]methylene]isobenzofuran-1-one (500 mg, 930.65 μmol) in THF (10 mL) was added N₂H₄·H₂O (174.71 mg, 2.79 mmol, 80% purity). The mixture was stirred at 70° C. for 3 hours. The reaction mixture was poured into water (10 mL), extracted with EtOAc (20 mL×3). The extracts were washed with brine, dried and concentrated to give 6-bromo-4-[[4-fluoro-3-[3-(trifluoromethyl)-6,8-dihydro-5H-[1,2,4]triazolo[4,3-a]pyrazine-7-carbonyl]phenyl]methyl]-2H-phthalazin-1-one (4(0) mg, crude) as a white solid. LCMS ESI mli 553.0 [M+H]⁺.

### Step 2:

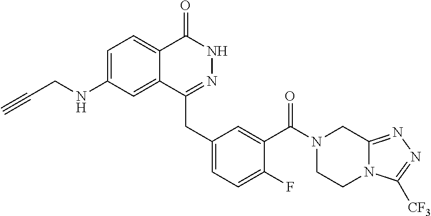 [SMILES: C#CCNc1ccc2c(=O)[nH]nc(Cc3ccc(F)c(C(=O)N4CCn5c(nnc5C(F)(F)F)C4)c3)c2c1]

### 4-(4-Fluoro-3-(3-(trifluoromethyl)-5,6,7,8-tetrahydro-[1,2,4]triazolo[4,3-a]pyrazine-7-carbonyl)benzyl)-6-(prop-2-yn-1-ylamino)phthalazin-1(2H)-one

To a solution of 6-bromo-4-[[4-fluoro-3-[3-(trifluoromethyl)-6,8-dihydro-5H-[1,2,4]triazolo[4,3-a]pyrazine-7-carbonyl]phenyl]methyl]-2H-phthalazin-1-one (100 mg, 181.39 μmol) in dioxane (3 mL) was added prop-2-yn-1-amine (59.94 mg, 1.09 mmol, 69.70 μL), t-BuONa (34.86 mg, 362.79 μmol). Pd(OAc)₂ (8.14 mg, 36.28 μmol) and Brettphos (19.47 mg, 36.28 μmol). The mixture was stirred at 100° C. under microwave for 1 hour. The mixture was purified by Prep-HPLC and Prep-TLC to give 4-[[4-fluoro-3-[3-(trifluoromethyl)-6,8-dihydro-5H-[1,2,4]triazolo[4,3-a]pyrazine-7-carbonyl]phenyl]methyl]-6-(prop-2-ynylamino)-2H-phthalazin-1-one (3.67 mg, 3.8% yield) as a white solid. LCMS ESI 527.7 [M+H]⁺. ¹H NMR (400 MHz, DMSO-d₆) δ 12.21 (s, 1H), 8.05 (d, J=8.7 Hz, 1H), 7.60 (s, 1H), 7.48 (d, J=5.2 Hz, 1H), 7.38 (t, J=9.0 Hz, 1H), 7.17 (td, J=8.7, 3.9 Hz, 2H), 6.91 (d. J=18.2 Hz, 1H), 5.10 (s, 1H), 4.80 (s, 1H), 4.30 (s, 3H), 4.07 (d, J=5.6 Hz, 3H), 3.79 (s, 1H), 3.15 (s, 1H), 2.16-2.01 (m, 1H).

### Synthesis of Example 186: 8-Fluoro-4-(4-(4-methylpiperazine-1-carbonyl)benzyl)phthalazin-1(2H)-one

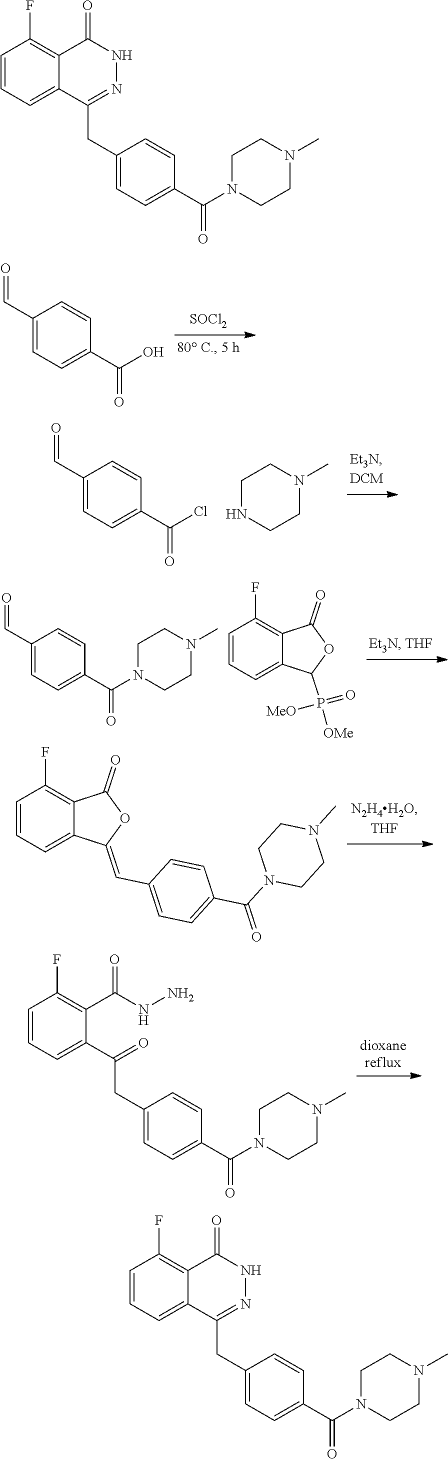

### Step 1

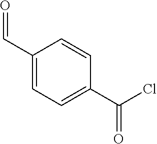 [SMILES: O=Cc1ccc(C(=O)Cl)cc1]

### 4-Formylbenzoyl chloride

4-Formylbenzoic acid (1.0 g, 6.66 mmol) was added slowly into sulfurous dichloride (3 mL) at 0° C. The mixture was stirred at 80° C. for 5 hr. The mixture was concentrated under reduced pressure to give 4-formylbenzoyl chloride (1.12 g, 100% yield) as a brown oil. LCMS ESI m/z: 541.1 [M+H]⁺.

### Step 2

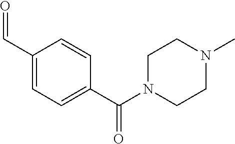 [SMILES: CN1CCN(C(=O)c2ccc(C=O)cc2)CC1]

### 4-(4-methylpiperazine-1-carbonyl)benzaldehyde

To a mixture of 4-formylbenzoyl chloride (1.12 g, 6.64 mmol) in dichloromethane (13 mL) was added triethylamine (807 mg, 7.97 mmol), 1-methylpiperazine (800 mg, 7.97 mmol) was added slowly. The resulting mixture was stirred at room temperature overnight. The mixture was quenched with methanol (50 mL) and concentrated under reduced pressure. The residue was purified by silica gel chromatography (methanol/dichloromethane=1/30, v/v) to give 4-(4-methylpiperazine-1-carbonyl)benzaldehyde (482 mg, 30.8% yield) as a white solid. ¹H NMR (400 MHz, DMSO-d6) δ 10.08 (s, 1H), 8.01 (d, J=8.1 Hz, 2H), 7.68 (d, J=8.0 Hz, 2H), 3.59 (s, 2H), 3.30-3.09 (m, 6H), 2.76 (s, 3H).

### Step 3, 4 and 5

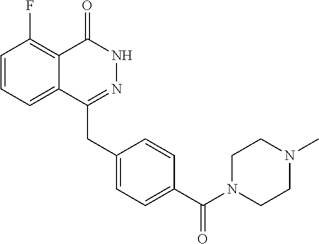 [SMILES: CN1CCN(C(=O)c2ccc(Cc3n[nH]c(=O)c4c(F)cccc34)cc2)CC1]

### 8-Fluoro-4-(4-(4-methylpiperazine-1-carbonyl)benzyl)phthalazin-1(2H)-one

Following the three-step procedure above in Example 6, but starting with 4-(4-methylpiperazine-1-carbonyl)benzaldehyde and dimethyl 4-fluoro-3-oxo-1,3-dihydroisobenzofuran-1-ylphosphonate gave the title compound as a white solid. LCMS ESI m/z: 380.9 [M+H]⁺. ¹H NMR (400 MHz, DMSO-d₆) δ 12.57 (s, 1H), 7.89 (td. J=8.1, 4.9 Hz, 1H), 7.77 (d, J=8.1 Hz, 1H), 7.59 (dd, J=11.3, 8.2 Hz, 1H), 7.37 (d, J=8.1 Hz, 2H), 7.31 (d, J=8.1 Hz, 2H), 4.32 (s, 2H), 3.48 (m, 4H), 2.41-2.22 (m, 4H), 2.19 (s, 3H).

### Synthesis of Example 187 and 188: 6-(4-(2-fluoro-5-(4-oxo-6-(prop-1-ynyl)-3,4-dihydrophthalazin-1-yloxy)benzoyl)piperazin-1-yl)nicotinonitrile and 6-(4-(2-fluoro-5-(4-oxo-7-(prop-1-ynyl)-3,4-dihydrophthalazin-1-yloxy)benzoyl)piperazin-1-yl)nicotinonitrile

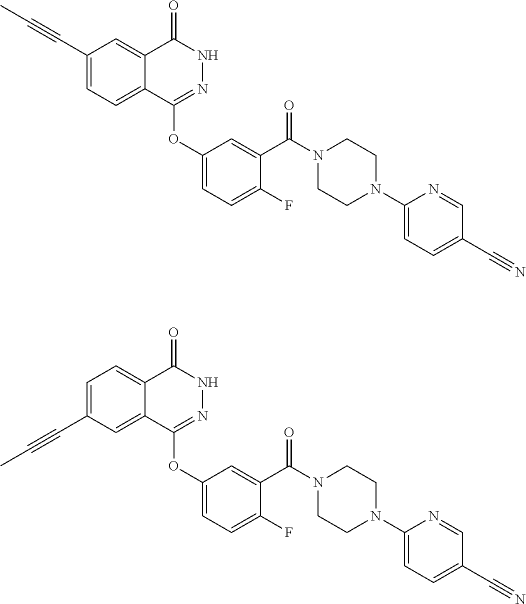 [SMILES: CC#Cc1ccc2c(Oc3ccc(F)c(C(=O)N4CCN(c5ccc(C#N)cn5)CC4)c3)n[nH]c(=O)c2c1 ; CC#Cc1ccc2c(=O)[nH]nc(Oc3ccc(F)c(C(=O)N4CCN(c5ccc(C#N)cn5)CC4)c3)c2c1]

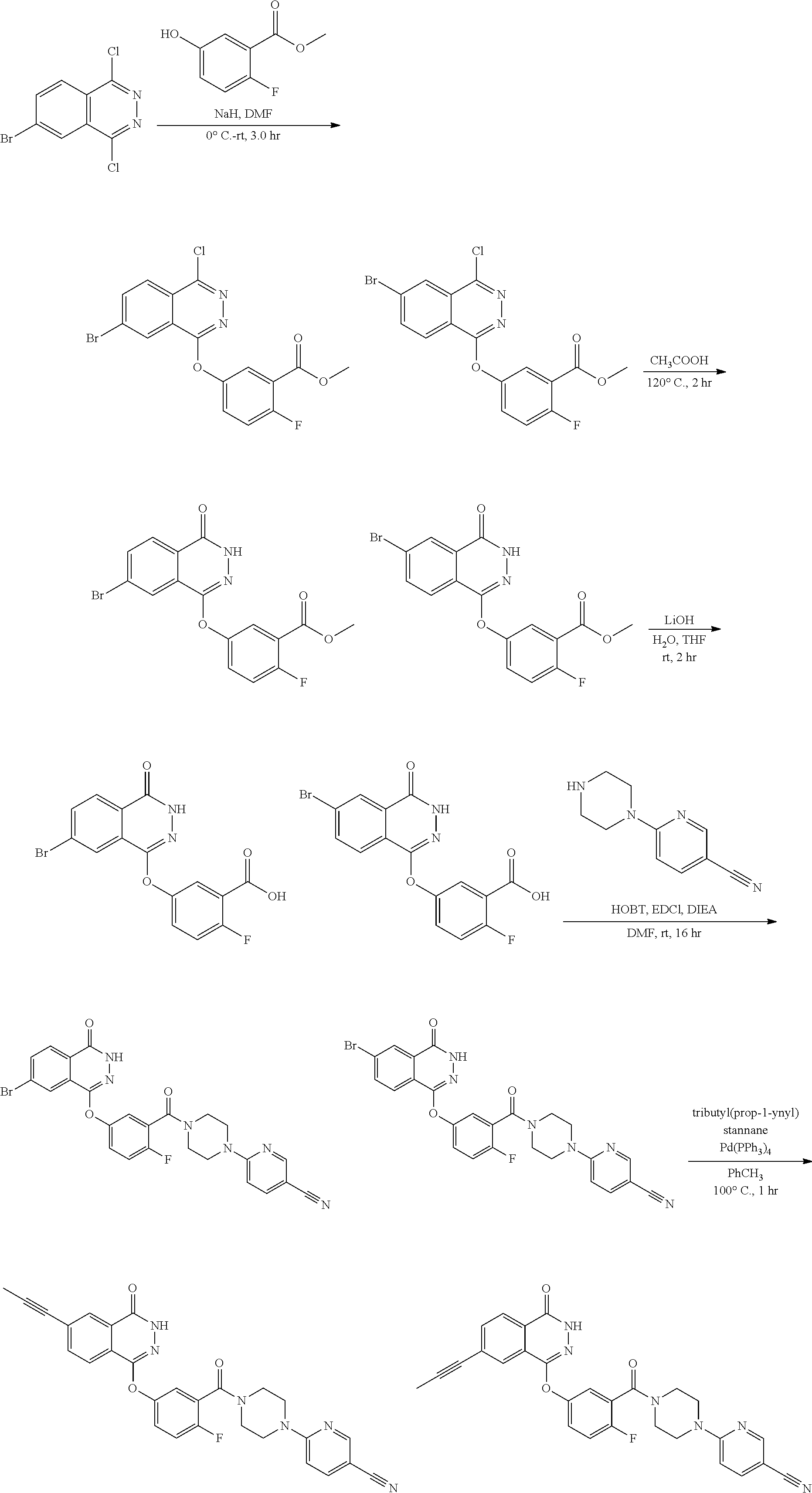

### Step 1

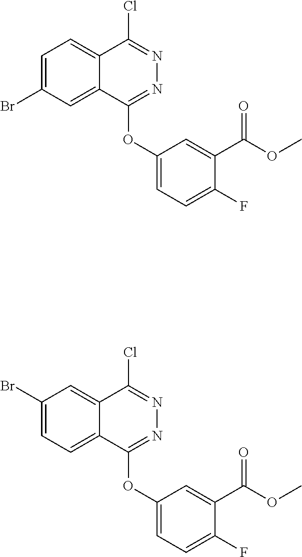 [SMILES: COC(=O)c1cc(Oc2nnc(Cl)c3ccc(Br)cc23)ccc1F ; COC(=O)c1cc(Oc2nnc(Cl)c3cc(Br)ccc23)ccc1F]

### methyl 5-(7-bromo-4-chlorophthalazin-1-yloxy)-2-fluorobenzoate and methyl 5-(6-bromo-4-chlorophthalazin-1-yloxy)-2-fluorobenzoate

To a solution of methyl 2-fluoro-5-hydroxy-benzoate (800 mg, 4.70 mmol) in DMF (15 mL) was added NaH (197.49 mg, 4.94 mmol, 60% purity) at 0° C. then the mixture was added dropwise to a solution of 6-bromo-4-dichloro-phthalazine (1.44 g, 5.17 mmol) in THF (60 mL) during 1 hr, then stirred at rt, for 3 hr. TLC (PE:EA=10:1) showed new spots. The reaction mixture was poured into H₂O (30 mL), extracted with EA (50 mL*3), washed with brine, dried over Na2SO₄ and concentrated. The residue was purified by silica gel chromatography to give the mixture of methyl 5-(7-bromo-4-chlorophthalazin-1-yloxy)-2-fluorobenzoate and methyl 5-(6-bromo-4-chlorophthalazin-1-yloxy)-2-fluorobenzoate (1.1 g, 56.8% yield) as a yellow solid and inseparable mixture, it will be used in the next step directly. For major isomer: ¹H NMR (400 MH z, DMSO-d₆) δ 8.70 (d, J=1.8 Hz, 1H), 8.44 (dd, J=8.8, 1.9 Hz, 1H), 8.27 (d, J=8.8 Hz, 1H), 7.97 (dd, J=6.0, 3.0 Hz, 1H), 7.80 (dd, J=5.2, 3.2 Hz, 1H), 7.58 (dd, J=10.3, 9.1 Hz, 1H), 3.93 (s, 3H),

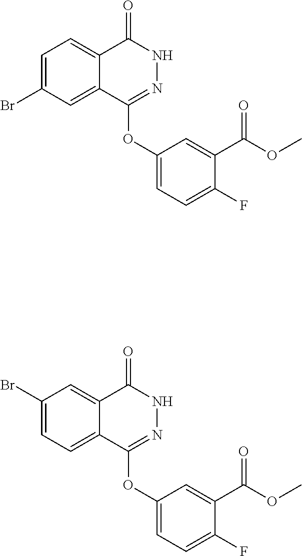 [SMILES: COC(=O)c1cc(Oc2n[nH]c(=O)c3ccc(Br)cc23)ccc1F ; COC(=O)c1cc(Oc2n[nH]c(=O)c3cc(Br)ccc23)ccc1F]

### Methyl 5-(7-bromo-4-oxo-3,4-dihydrophthalazin-1-yloxy)-2-fluorobenzoate and methyl 5-(6-bromo-4-oxo-3,4-dihydrophthalazin-1-yloxy)-2-fluorobenzoate

To a 100 mL round bottle was added the mixture of methyl 5-(7-bromo-4-chloro-phthalazin-1-yl)oxy-2-fluoro-benzoate and methyl 5-(6-bromo-4-chloro-phthalazin-1-yl)oxy-2-fluoro-benzoate (800 mg, 1.94 mmol), then CH₃COOH (20 mL) was added. The reaction mixture was heated to 120° C. for 2 hr. The reaction mixture was concentrated to give a residue, poured into H₂O (20 mL), stirred at rt, for 2 minutes. The solids were collected by filtration and dried under vacuum to give the mixture of methyl 5-(7-bromo-4-oxo-3,4-dihydrophthalazin-1-yloxy)-2-fluorobenzoate and methyl 5-(6-bromo-4-oxo-3,4-dihydrophthalazin-1-yloxy)-2-fluorobenzoate as an inseparable mixture (760 mg, 99% yield) as a white solid. LCMS ESI m/z: 390.7 [M−H]⁻.

### Step 3

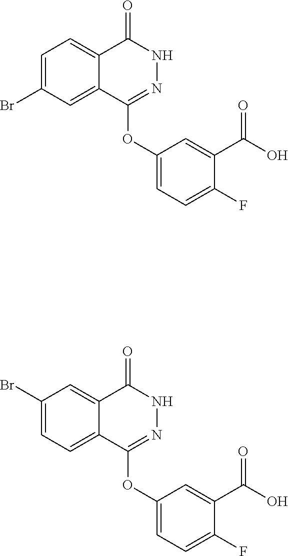 [SMILES: O=C(O)c1cc(Oc2n[nH]c(=O)c3ccc(Br)cc23)ccc1F ; O=C(O)c1cc(Oc2n[nH]c(=O)c3cc(Br)ccc23)ccc1F]

### 5-(7-Bromo-4-oxo-3,4-dihydrophthalazin-1-yloxy)-2-fluorobenzoic acid and 5-(6-bromo-4-oxo-3,4-dihydrophthalazin-1-yloxy)-2-fluorobenzoic acid

To a solution of the mixture of methyl 5-(7-bromo-4-oxo-3,4-dihydrophthalazin-1-yloxy)-2-fluorobenzoate and methyl 5-(6-bromo-4-oxo-3,4-dihydrophthalazin-1-yloxy)-2-fluorobenzoate (760 mg, 1.93 mmol) in H₂O (10 mL) and THF (10 mL) was added LiOH (138.89 mg, 5.80 mmol), then stirred at rt. for 2 hr. The mixture was washed with DCM, the aqueous layer was collected and acidified with 1 M HCl aq. until the solution reached pH 5. The solids were collected by filtration and dried under vacuum to give 5-(7-bromo-4-oxo-3,4-dihydrophthalazin-1-yloxy)-2-fluorobenzoic acid and 5-(6-bromo-4-oxo-3,4-dihydrophthalazin-1-yloxy)-2-fluorobenzoic acid as an inseparable mixture (580 mg, 79% yield) as a white solid. LCMS ESI m/z: 390.7 [M−H]⁻.

### Step 4 and 5

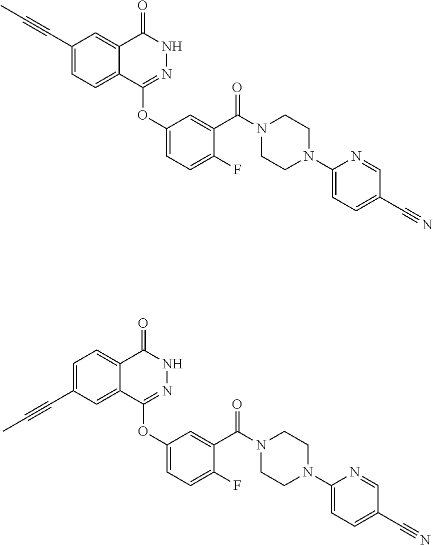 [SMILES: CC#Cc1ccc2c(Oc3ccc(F)c(C(=O)N4CCN(c5ccc(C#N)cn5)CC4)c3)n[nH]c(=O)c2c1 ; CC#Cc1ccc2c(=O)[nH]nc(Oc3ccc(F)c(C(=O)N4CCN(c5ccc(C#N)cn5)CC4)c3)c2c1]

### 6-(4-(2-fluoro-5-(4-oxo-6-(prop-1-ynyl)-3,4-dihydrophthalazin-1-yloxy)benzoyl)piperazin-1-yl)nicotinonitrile and 6-(4-(2-fluoro-5-(4-oxo-7-(prop-1-ynyl)-3,4-dihydrophthalazin-1-yloxy)benzoyl)piperazin-1-yl)nicotinonitrile

Following the general two-step procedure above in Example 1, but starting with the mixture of 5-(7-bromo-4-oxo-3,4-dihydrophthalazin-1-yloxy)-2-fluorobenzoic acid and 5-(6-bromo-4-oxo-3,4-dihydrophthalazin-1-yloxy)-2-fluorobenzoic acid gave the mixture of title compound (80 mg) as a white solid. The mixture was purified by prepare TLC (toluene/DCM/THF=1/6/1. Petroleum ether/toluene/DCM/THF=1/2/3/2 to petroleum ether/toluene/DCM/THF=1/2/2/2) to give 6-(4-(2-fluoro-5-(4-oxo-6-(prop-1-ynyl)-3,4-dihydrophthalazin-1-yloxy)benzoyl)piperazin-1-yl)nicotinonitrile (13 mg) and 6-(4-(2-fluoro-5-(4-oxo-7-(prop-1-ynyl)-3,4-dihydrophthalazin-1-yloxy)benzoyl)piperazin-1-yl)nicotinonitrile (10.8 mg) as white solids. LCMS ESI m/z: 509.2 [M+H]⁺.

6-(4-(2-fluoro-5-(4-oxo-6-(prop-1-ynyl)-3,4-dihydrophthalazin-1-yloxy)benzoyl)piperazin-1-yl)nicotinonitrile ¹H NMR (400 MHz, DMSO-d₆) δ 12.06 (s, 1H), 8.51 (d, J=2.2 Hz, 1H), 8.14 (d, J=1.2 Hz, 1H), 8.07 (d, J=8.3 Hz, 1H), 7.95 (dd, J=8.3, 1.6 Hz, 1H), 7.90 (dd, J=9.1, 2.3 Hz, 1H), 7.49-7.38 (m, 3H), 6.94 (d, J=9.1 Hz, 1H), 3.81-3.66 (m, 6H), 3.38 (s, 2H), 2.14 (s, 3H).

6-(4-(2-fluoro-5-(4-oxo-7-(prop-1-ynyl)-3,4-dihydrophthalazin-1-yloxy)benzoyl)piperazin-1-yl)nicotinonitrile ¹H NMR (400 MHz, DMSO-d₆) δ 12.03 (s, 1H), 8.51 (d, J=2.1 Hz, 1H), 8.21 (d, J=8.2 Hz, 1H), 8.03 (s, 1H), 7.94-7.87 (m, 2H), 7.52-7.38 (m, 3H), 6.94 (d, J=9.1 Hz, 1H), 3.81-3.67 (m, 6H), 3.39 (s, 2H), 2.13 (s, 3H).

### Synthesis of Example 189: 2-((1-((5-((7-cyclobutoxy-4-oxo-3,4-dihydrophthalazin-1-yl)methyl)-2-fluorophenyl) sulfonyl)azetidin-3-yl)(methyl)amino)isonicotinonitrile

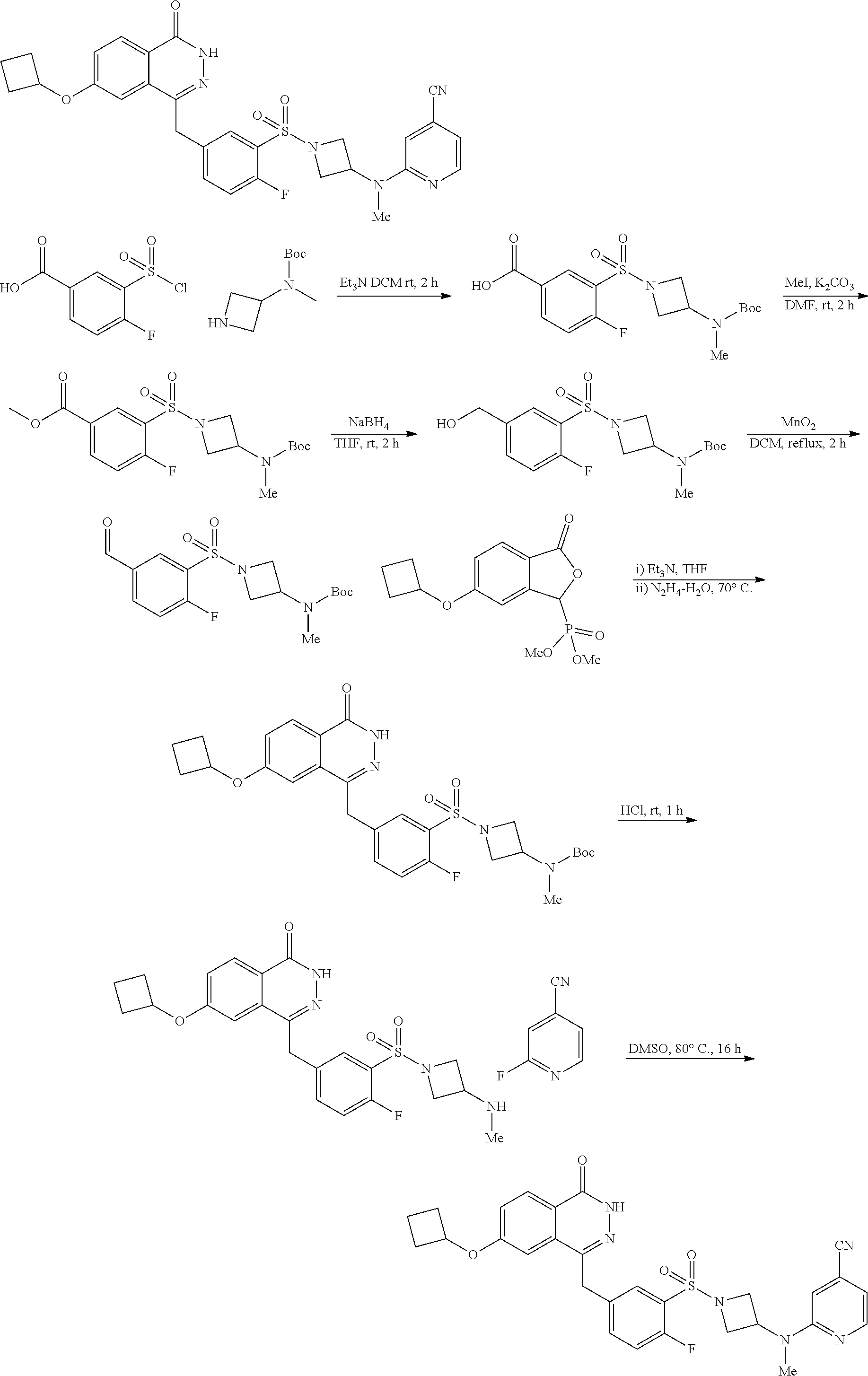

### Step 1

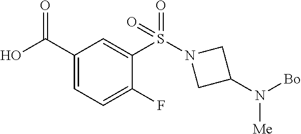

### 3-((3-((tert-butoxycarbonyl)(methyl)amino)azetidin-1-yl)sulfonyl)-4-fluorobenzoic acid

A solution of 3-(chlorosulfonyl)-4-fluorobenzoic acid (1 g, 4.19 mmol), tert-butyl azetidin-3-yl(methyl)carbamate (780 mg, 4.19 mmol) and Et₃N (1.7 g, 16.76 mmol) in THF (10 mL) was stirred at rt for 2 hours. The solution was concentrated in vacuo, then diluted with water (50 mL) and extracted with EtOAc (30 mL×3). The combined organic layer was washed with brine, dried over anhydrous Na₂SO₄, filtered and concentrated in vacuo. The residue was purified by silica gel chromatography (DCM/MEOH=10/1) to give 3-((3-((tert-butoxycarbonyl)(methyl)amino)azetidin-1-yl)sulfonyl)-4-fluorobenzoic acid (1.40 g, 88% yield) as a white solid. LCMS ESI m/z: 389.1 [M+H]⁺.

### Step 2

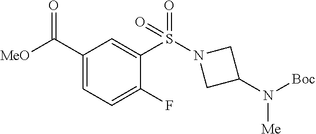 [SMILES: COC(=O)c1ccc(F)c(S(=O)(=O)N2CC(N(C)C(=O)OC(C)(C)C)C2)c1]

### Methyl 3-(3-((tert-butoxycarbonylmethyl)amino)azetidin-1-yl)sulfonyl)-4-fluorobenzoate

To a mixture of 3-((3-((tert-butoxycarbonyl)(methyl)amino)azetidin-1-yl)sulfonyl)-4-fluorobenzoic acid (1.4 g, 3.71 mmol) and K₂CO₃ (768 mg, 5.56 mmol) in DMF (15 mL) was added Mei (631 mg, 4.45 mmol). The reaction was stirred at rt for 2 hours, then diluted with water (50 mL) and extracted with EtOAc (20 mL×3). The combined organic layer was washed with brine, dried over anhydrous Na₂SO₄, filtered and concentrated in vacuo. The residue was purified by silica gel chromatography (PE/EA=1/1) to afford methyl 3-((3-((tert-butoxycarbonyl) (methyl) amino) azetidin-1-yl)sulfonyl)-4-fluorobenzoate (1.10 g, 65% yield) as a colorless oil. LCMS ESI m/z: 403.1 [M+H]⁺.

### Step 3

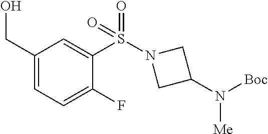 [SMILES: CN(C(=O)OC(C)(C)C)C1CN(S(=O)(=O)c2cc(CO)ccc2F)C1]

### tert-butyl (1-((2-fluoro-5-(hydroxymethyl)phenyl)sulfonyl)azetidin-3-yl)(methyl) carbamate

To a solution of methyl 3-((3-((tert-butoxycarbonyl)(methyl)amino)azetidin-1-yl)sulfonyl)-4-fluorobenzoate (1.1 g, 2.73 mmol) in THF (10 mL) was added NaBH₄ (1.5 g, 40 mmol). The mixture was stirred at rt for 2 hours, then diluted with water (50 mL) and extracted with EtOAc (30 mL×3). The combined organic layer was dried over Na₂SO₄, filtered and concentrated in vacuum. The residue was purified by silica gel chromatography (PE/EA=1/1) to afford tert-butyl (1-((2-fluoro-5-(hydroxymethyl) phenyl) sulfonyl)azetidin-3-yl)(methyl)carbamate (780 mg, 65% yield) as a yellow oil. LCMS ESI m/z: 375.0 [M+H]⁺.

### Step 4

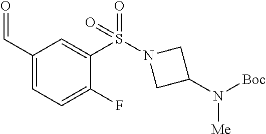 [SMILES: CN(C(=O)OC(C)(C)C)C1CN(S(=O)(=O)c2cc(C=O)ccc2F)C1]

### tert-butyl (1-((2-fluoro-5-formylphenyl)sulfonyl)azetidin-3-yl)(methyl)carbamate

A mixture of tert-butyl (1-((2-fluoro-5-(hydroxymethyl)phenyl)sulfonyl)azetidin-3-yl)(methyl)carbamate (780 mg, 2.08 mmol) and MnO₂ (2.71 g, 27.94 mmol) in DCM (20 mL) was stirred under reflux for 2 hours. The reaction mixture was cooled to rt and filtered. The filtrate was concentrated in vacuo to give tert-butyl (1-((2-fluoro-5-formylphenyl)sulfonyl)azetidin-3-yl)(methyl)carbamate (650 mg, 83% yield) as a white solid. LCMS ESI m/z: 373.1 [M+H]⁺. This was used without further purification.

### Step 5

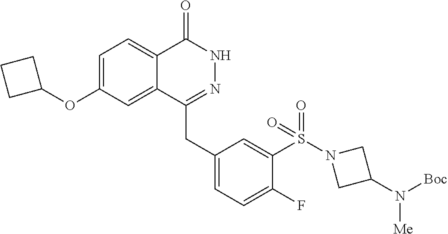 [SMILES: CN(C(=O)OC(C)(C)C)C1CN(S(=O)(=O)c2cc(Cc3n[nH]c(=O)c4ccc(OC5CCC5)cc34)ccc2F)C1]

### tert-butyl (1-((5-((7-cyclobutoxy-4-oxo-3,4-dihydrophthalazin-1-yl)methyl)-2-fluorophenyl)sulfonyl)azetidin-3-yl)(methyl)carbamate

A solution of tert-butyl (1-((2-fluoro-5-formylphenyl)sulfonyl)azetidin-3-yl)(methyl) carbamate (650 mg, 1.75 mmol) and methyl dimethyl (6-cyclobutoxy-3-oxo-1,3-dihydroisobenzofuran-1-yl)phosphonate (546 mg, 1.75 mmol) in anhydrous THF (6 mL) was stirred at 25° C. under Ar (g) for 3 hours. Hydrazine hydrate (130 mg, 2.62 mmol) was added and the reaction was stirred at 70° C. for 2 hours. The reaction was cooled to rt, then THF was removed under vacuum. The formed solids were washed with water (10 mL) and petroleum ether/EA (10:1, 10 mL) to give tert-butyl (1-((5-((7-cyclobutoxy-4-oxo-3,4-dihydrophthalazin-1-yl)methyl)-2-fluorophenyl)sulfonyl)azetidin-3-yl)(methyl)carbamate (640 mg, 64% yield) as a yellow solid. LCMS ESI m/z: 573.2 [M+H]⁺.

### Step 6

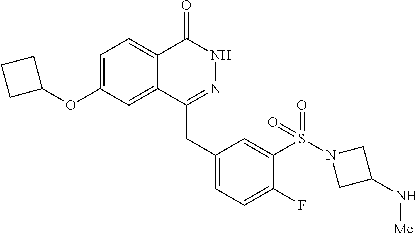 [SMILES: CNC1CN(S(=O)(=O)c2cc(Cc3n[nH]c(=O)c4ccc(OC5CCC5)cc34)ccc2F)C1]

### 6-cyclobutoxy-4-(4-fluoro-3-((3-(methylamino)azetidin-1-yl)sulfonyl)benzyl) phthalazin-1(2H)-one

To a solution of tert-butyl (1-((5-((7-cyclobutoxy-4-oxo-3,4-dihydrophthalazin-1-yl)methyl)-2-fluorophenyl)sulfonyl)azetidin-3-yl)(methyl)carbamate (640 mg, 1.12 mmol) in dioxane (5 mL) was added HCl/dioxane (4M, 10 mL) and stirred at 25° C. for 1 hour. The mixture was concentrated in vacuo to afford 6-cyclobutoxy-4-(4-fluoro-3,4(3-(methylamino)azetidin-1-yl)sulfonyl)benzyl)phthalazin-1(2H)-one (528 mg, 100% yield) as a white solid. LCMS ESI m/z: 473.1 [M+H]⁺. This was used without further purification.

### Step 7

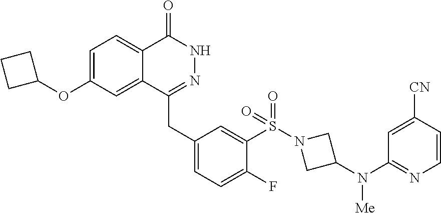 [SMILES: CN(c1cc(C#N)ccn1)C1CN(S(=O)(=O)c2cc(Cc3n[nH]c(=O)c4ccc(OC5CCC5)cc34)ccc2F)C1]

### 2-((1-((5-((7-cyclobutoxy-4-oxo-3,4-dihydrophthalazin-1-yl)methyl)-2-fluorophenyl) sulfonyl)azetidin-3-yl)(methyl)amino)isonicotinonitrile

A solution of 6-cyclobutoxy-4-(4-fluoro-3-((3-(methylamino)azetidin-1-yl)sulfonyl) benzyl)phthalazin-1(2H)-one (140 mg, 0.28 mmol), 2-fluoroisonicotinonitrile (140 mg, 11.38 mmol) in DMSO (2 mL) was stirred at 80° C. for 16 hours. The reaction was purified by prep-HPLC to give 2-((1-((5-((7-cyclobutoxy-4-oxo-3,4-dihydrophthalazin-1-yl)methyl)-2-fluorophenyl)sulfonyl)azetidin-3-yl)(methyl)amino)isonicotinonitrile (57 mg, 33% yield) as a white solid. LCMS ESI m/z: 575.1 [M+H]⁺. 1H-NMR (400 MHz, DMSO-d₆) δ 12.47 (s, 1H), 8.41 (d, J=3.0 Hz, 1H), 8.15 (d, J=8.8 Hz, 1H), 7.83 (s, 1H), 7.60 (d, J=5.1 Hz, 1H), 7.47-7.37 (m, 1H), 7.30 (dd, J=8.8, 2.3 Hz, 1H), 7.21 (d, J=4.1 Hz, 1H), 7.18-7.08 (m, 2H), 4.88-4.78 (m, 1H), 4.75-4.64 (m, 1H), 4.61-4.49 (m, 1H), 4.42-4.16 (m, 4H), 3.25-3.17 (m, 1H), 2.96 (s, 3H), 2.45-2.35 (m, 2H), 2.08-1.93 (m, 2H), 1.84-1.73 (m, 1H), 1.72-1.61 (m, 1H).

### Synthesis of Example 190: 6-(4-(2-fluoro-5-((4-oxo-3,4-dihydrophthalazin-1-yl)oxy)benzoyl)piperazin-1-yl)nicotinonitrile

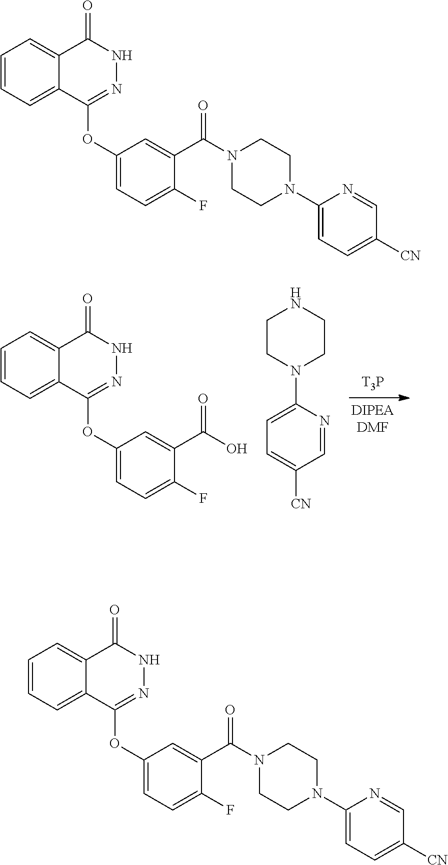

To a solution of 2-fluoro-5-((4-oxo-3,4-dihydrophthalazin-1-yl)oxy)benzoic acid (0.062 g, 0.2 mmol) synthesized following Example 185 and 186 procedures and 6-(piperazin-1-yl)nicotinonitrile (0.040 g, 0.2 mmol) in anhydrous DMF (1.0 mL) at 0° C. was added diisopropylethylamine (0.10 mL, 0.6 mmol), followed by addition of propylphosphonic anhydride (T3P) (0.25 mL, 0.4 mmol) (50% in EtOAc). The mixture was stirred for 30 min at rt. Upon completion of the reaction, it was dissolved in ethyl acetate (5 mL) and added with sat. NaHCO₃ (5 mL). The layers were separated and the aqueous layer was extracted with ethyl acetate. The organic extracts were washed with brine (5 mL), filtered through a silica plug and dried over MgSO₄. After filtration, the solvent was removed in vacuo. Flash chromatography over silica gel (0 to 100% EtOAc in Heptanes) afforded the product as a white solid (0.0274 g, 28% yield). LCMS ESI m/z 471.2 [M+H]⁺. ¹H NMR (400 MHz, CDCl₃) δ 10.20 (s, 1H), 8.40-8.32 (m, 2H), 8.11-8.04 (m, 1H), 7.83 (dtd, J=23.1, 7.4, 1.3 Hz, 2H), 7.57 (dd, J=9.0, 2.3 Hz, 1H), 7.31-7.22 (m, 2H), 7.17-7.07 (m, 1H), 6.60-6.55 (m, 1H), 3.84 (s, 2H), 3.71 (dq, J=12.0, 6.8, 6.0 Hz, 4H), 3.63-3.45 (m, 2H).

### Synthesis of Example 191: 4-(((4-Oxo-4-(4-(5-(trifluoromethyl)pyrimidin-2-yl)piperazin-1-yl)butan-2-yl)amino)methyl)-6-(prop-1-yn-1-yl)phthalazin-1(2H)-one

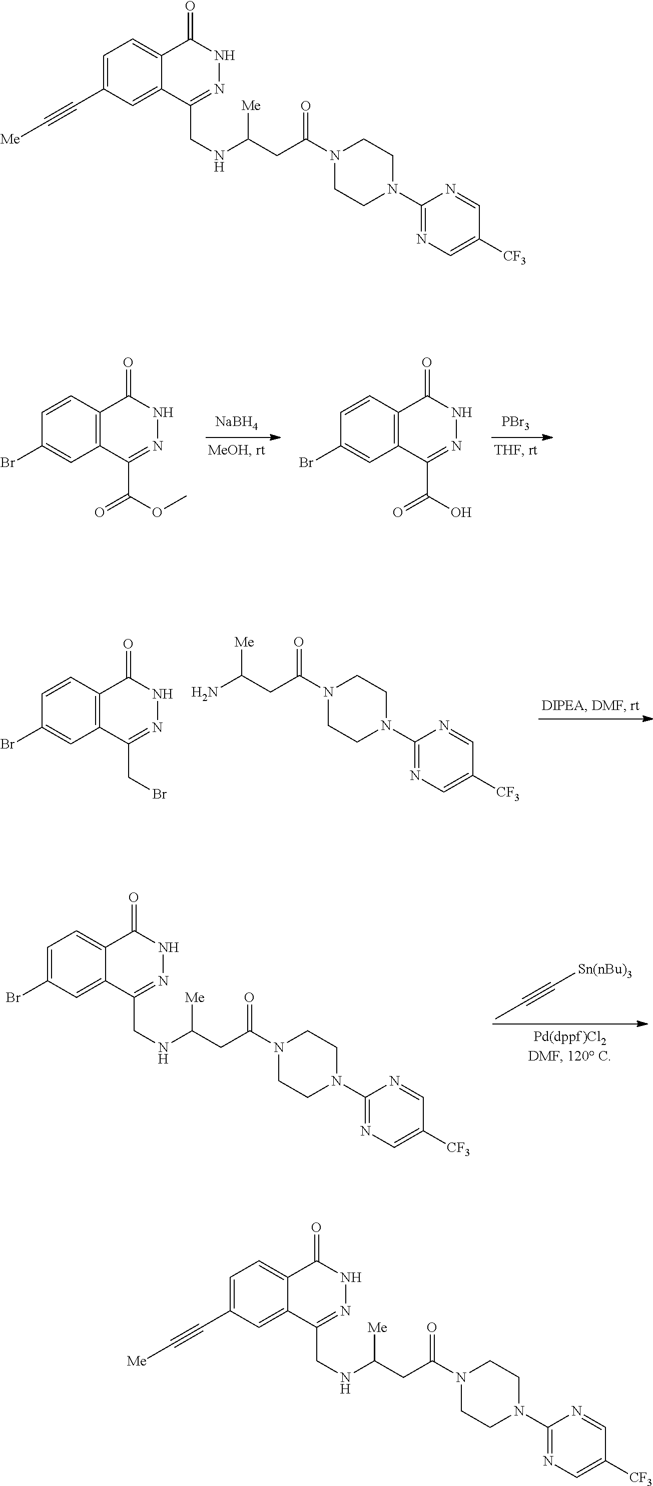

### Step 1

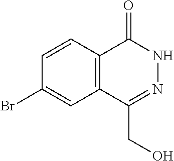 [SMILES: O=c1[nH]nc(CO)c2cc(Br)ccc12]

### 6-Bromo-4-(hydroxymethyl)phthalazin-1(2H)-one

To a solution of methyl 7-bromo-4-oxo-3,4-dihydrophthalazine-1-carboxylate (600 mg, 2.12 mmol) in MeOH (5 mL) was added NaBH₄ (802 mg, 21.20 mmol), the mixture was stirred at rt for 16 hours. The solvent was removed in vacuo, the residue was diluted with H₂O (10 mL), aq 6N HCl was added until the solution reached pH 4. The solid was collected by filtration and dried under vacuum to give 6-bromo-4-(hydroxymethyl)-2H-phthalazin-1-one (358 mg, 1.40 mmol, 66% yield) as a white solid. LCMS ESI m/z: 255.1 [M+H]J.

### Step 2

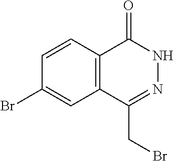 [SMILES: O=c1[nH]nc(CBr)c2cc(Br)ccc12]

### 6-Bromo-4-(bromomethyl)phthalazin-1(2H)-one

To a solution of 6-bromo-4-(hydroxymethyl)-2H-phthalazin-1-one (258 mg, 1.01 mmol) in THF (10 mL) was added PBr₃ (411 mg, 1.52 mmol) slowly, the mixture was stirred at rt for 0.5 h. The reaction was quenched with water (10 mL) and extracted with EtOAc (10 mL×3). The combined organic phase was washed with sat. NaCl solution, dried over Na₂SO₄, filtered and concentrated in vacuo to give 6-bromo-4-(bromomethyl)phthalazin-1(2H)-one (226 mg, 70% yield) as a white solid. LCMS ESI m/z: 317.1 [M+H]⁺.

### Step 3

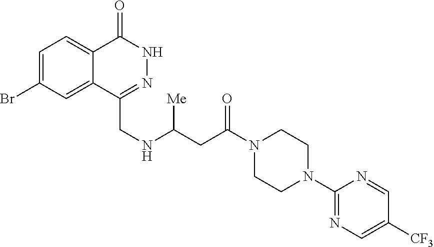 [SMILES: CC(CC(=O)N1CCN(c2ncc(C(F)(F)F)cn2)CC1)NCc1n[nH]c(=O)c2ccc(Br)cc12]

### 6-Bromo-4-(((4-oxo-4-(4-(5-(trifluoromethyl)pyrimidin-2-yl)piperazin-1-yl)butan-2-yl)amino)methyl)phthalazin-1(2H)-one

A solution of 6-bromo-4-(bromomethyl)phthalazin-1(2H)-one (276 mg, 0.89 mmol), 3-amino-1-(4-(5-(trifluoromethyl)pyrimidin-2-yl)piperazin-1-yl)butan-1-one (307 mg, 0.88 mmol) and DIPEA (337 mg, 2.60 mmol) in DMF (3 mL) was stirred at rt for 1 hour. The mixture was purified by prep-HPLC to give 6-bromo-4-(((4-oxo-4-(4-(5-(trifluoromethyl)pyrimidin-2-yl)piperazin-1-yl)butan-2-yl)amino)methyl)phthalazin-1(2H)-one (130 mg, 27% yield) as a white solid. LCMS ESI m/z: 554.1 [M+H]⁺.

### Step 4

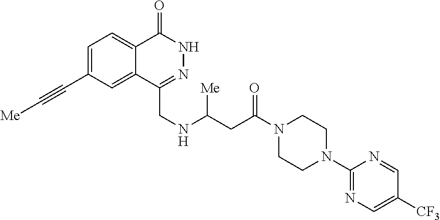 [SMILES: CC#Cc1ccc2c(=O)[nH]nc(CNC(C)CC(=O)N3CCN(c4ncc(C(F)(F)F)cn4)CC3)c2c1]

### 4-(((4-Oxo-4-(4-(5-(trifluoromethyl)pyrimidin-2-yl)piperazin-1-yl)butan-2-yl)amino)methyl)-6-(prop-1-yn-1-yl)phthalazin-1(2H)-one

To a solution of 6-bromo-4-[[[1-methyl-3-oxo-3-[4-[5-(trifluoromethyl)pyrimidin-2-yl]piperazin-1-yl]propyl]amino]methyl]-2H-phthalazin-1-one (110 mg, 0.20 mmol) in 1,4-dioxane (10 mL) was added Pd(dppf)Cl₂ (15 mg, 0.20 mmol) and tributyl(prop-1-ynyl)stannane (196 mg, 0.60 mmol), then the mixture was stirred at 120° C. under Ar atmosphere for 2 hours. The mixture was purified by prep-HPLC to afford 4-(((4-oxo-4-(4-(5-(trifluoromethyl)pyrimidin-2-yl)piperazin-1-yl)butan-2-yl)amino)methyl)-6-(prop-1-yn-1-yl)phthalazin-1(2H)-one (75 mg, 0.13 mmol, 68% yield) as a white solid. LCMS ESI m/z: 514.2 [M+H]⁺. ¹H NMR (400 MHz, CDCl₃) δ 12.61 (s, 1H), 8.73 (s, 2H), 8.17 (d, J=7.8 Hz, 1H), 8.14 (s, 1H), 8.11 (s, 1H), 7.75 (dd, J=8.2, 1.3 Hz, 1H), 4.13 (d, J=13.8 Hz, 1H), 3.98 (d, J=13.8 Hz, 1H), 3.85-3.70 (m, 6H), 3.51 (d, J=4.5 Hz, 2H), 3.15 (dd, J=12.5, 6.3 Hz, 2H), 2.63 (dd, J=15.2, 7.0 Hz, 1H), 2.44-2.34 (m, 1H), 2.10 (s, 3H), 1.15 (d, J=6.3 Hz, 3H).

### Synthesis of Example 192: 4-(1-(3-Oxo-3-(4-(5-(trifluoromethyl)pyrimidin-2-yl)piperazin-1-yl)propoxy)ethyl)-6-(prop-1-ynyl)phthalazin-1(2H)-one

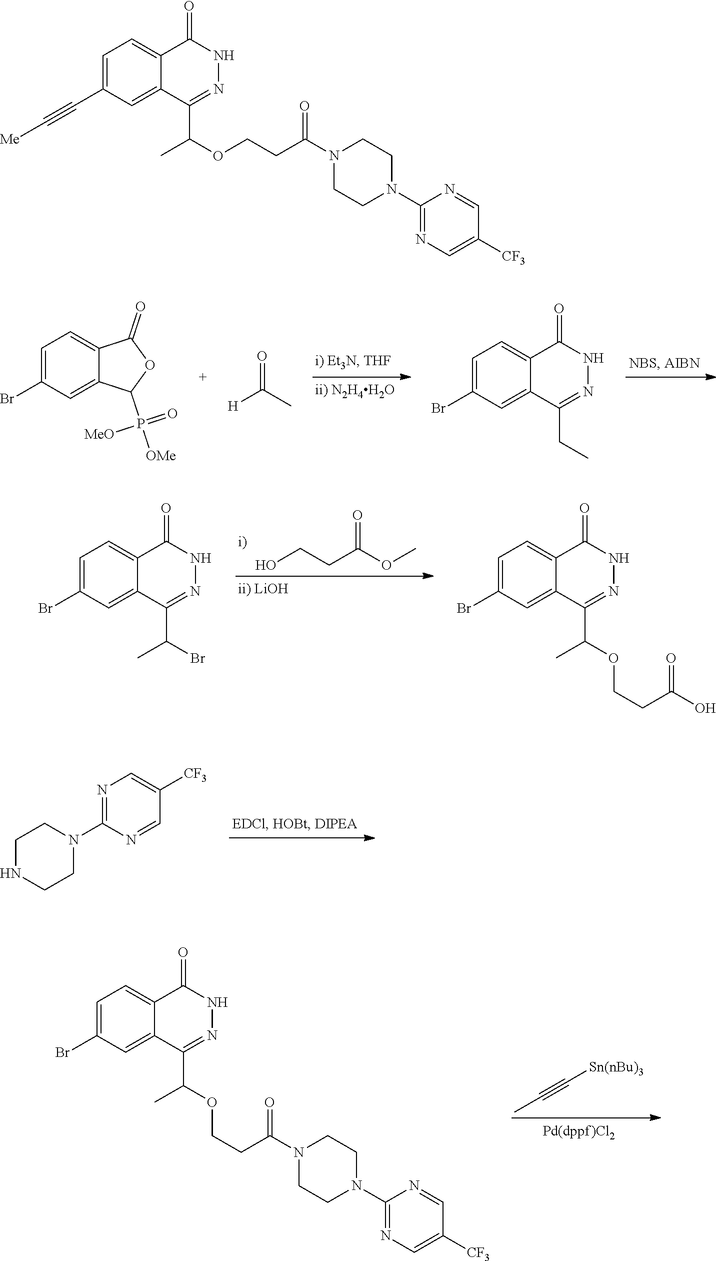

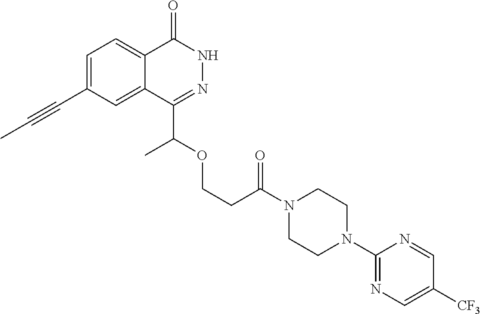 [SMILES: CC#Cc1ccc2c(=O)[nH]nc(C(C)OCCC(=O)N3CCN(c4ncc(C(F)(F)F)cn4)CC3)c2c1]

### Step 1

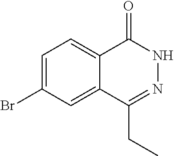

### 6-Bromo-4-ethylphthalazin-1(2H)-one

A mixture of dimethyl 6-bromo-3-oxo-1,3-dihydroisobenzofuran-1-ylphosphonate (1.00 g, 3.13 mmol), acetaldehyde (275 mg, 6.26 mmol) and Et₃N (947 mg, 9.39 mmol) in THF (10 mL) was stirred at rt for 12 hours. To the reaction mixture, N₂H₄·H₂O (368 mg, 6.26 mmol) was added and stirred at 70° C. for 2 hours. The reaction was cooled to rt, concentrated in vacuo and the residue was purified by silica gel chromatography (EtOAc/petroleum ether=0 to 20%) to give 6-bromo-4-ethylphthalazin-1(2H)-one as a yellow solid (790 mg, 99% yield). LCMS ESI m/z: 253 [M+H]⁺. ¹HNMR (400 MHz, DMSO-d₆) δ 12.58 (s, 1H), 8.18 (s, 1H), 8.16 (d, J=8.8 Hz, 1H), 8.01 (dd, J=8.4, 1.6 Hz, 1H), 2.99 (q, J=14.8, 7.2 Hz, 2H), 1.23 (t, J=7.2 Hz, 3H).

### Step 2

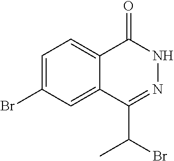

### 6-Bromo-4-(1-bromoethyl)phthalazin-1(2H)-one

A mixture of 5-bromo-7-(trifluoromethyl)isobenzofuran-1(3H)-one (790 mg, 3.12 mmol), NBS (667 mg, 3.74 mmol) and AIBN (102 mg, 0.62 mmol) in CCl₄ (10 mL) was stirred at 80° C. for 8 hours. The mixture was concentrated in vacuo, and the residue was purified by silica gel chromatography (EtOAc/petroleum ether=0 to 20%) to give 6-bromo-4-(1-bromoethyl)phthalazin-1(2H)-one as a yellow solid (460 mg, 45% yield). LCMS ESI m/z: 331 [M+H]⁺. ¹H NMR (400 MHz, DMSO-d₆) δ 12.68 (s, 1H), 8.36 (s, 1H), 8.19 (d, J=8.8 Hz, 1H), 8.04 (dd, J=8.4, 1.6 Hz, 1H), 6.10 (q, J=6.4 Hz, 1H), 2.05 (d, J=6.4 Hz, 3H).

### Step 3

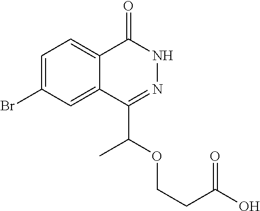 [SMILES: CC(OCCC(=O)O)c1n[nH]c(=O)c2ccc(Br)cc12]

### 3-(1-(7-Bromo-4-oxo-3,4-dihydrophthalazin-1-yl)ethoxy)propanoic acid

A solution of 6-bromo-4-(1-bromoethyl)phthalazin-1(2H)-one (460 mg, 1.39 mmol) in methyl 3-hydroxypropanoate (0.6 mL) were stirred at 100° C. for 5 hours. The reaction was cooled to rt, diluted with H₂O (10 mL) and extracted with EtOAc (3×20 mL). The combined organic layer was dried over Na₂SO₄, filtered and concentrated in vacuo. The residue was dissolved in MeOH (3 mL), then added a solution of LiOH (580 mg, 13.9 mmol) in H₂O (1 mL). The reaction mixture was stirred at rt for 6 hours, then aq HCl (1N) was added until the solution reached pH 5˜6. The mixture was extracted with EtOAc (3×20 mL). The combined organic layer was dried over Na₂SO₄, filtered and concentrated in vacuo. The residue was purified by silica gel chromatography (EtOAc/petroleum ether=60 to 80%) to give 3-(1-(7-bromo-4-oxo-3,4-dihydrophthalazin-1-yl)ethoxy)propanoic acid as a yellow solid (210 mg, 38% yield). LCMS ESI m/z: 341 [M+H]⁺.

### Step 4

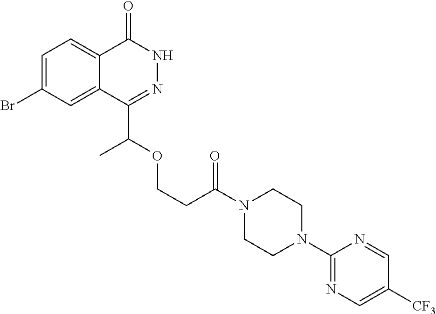 [SMILES: CC(OCCC(=O)N1CCN(c2ncc(C(F)(F)F)cn2)CC1)c1n[nH]c(=O)c2ccc(Br)cc12]

### 6-Bromo-4-(1-(3-oxo-3-(4-(5-(trifluoromethyl)pyrimidin-2-yl)piperazin-1-yl)propoxy)ethyl)phthalazin-1(2H)-one

A solution of 3-(1-(7-bromo-4-oxo-3,4-dihydrophthalazin-1-yl)ethoxy)propanoic acid (210 mg, 0.62 mmol), EDCI (118 mg, 0.62 mmol), 2-(piperazin-1-yl)-5-(trifluoromethyl)pyrimidine (145 mg, 0.62 mmol). HOBT (126 mg, 0.93 mmol) and DIPEA (320 mg, 2.48 mmol) in DMF (5 mL) were stirred at rt for 6 hours. The mixture was diluted with H₂O (5 mL) and extracted with EtOAc (3×20 mL). The combined organic layer was dried over Na₂SO₄, filtered and concentrated in vacuo. The residue was purified by silica gel chromatography (EtOAc/petroleum ether=80 to 100%) to give 6-bromo-4-(1-(3-oxo-3-(4-(5-(trifluoromethyl)pyrimidin-2-yl)piperazin-1-yl)propoxy)ethyl)phthalazin-1(2H)-one as a white solid (260 mg, 76% yield). LCMS ESI m/z: 555 [M+H]⁺.

### Step 5

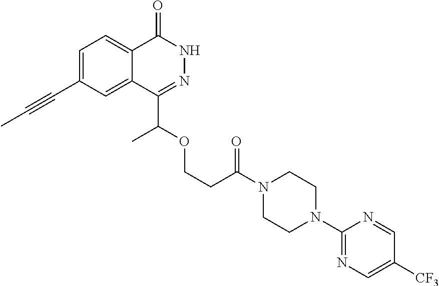 [SMILES: CC#Cc1ccc2c(=O)[nH]nc(C(C)OCCC(=O)N3CCN(c4ncc(C(F)(F)F)cn4)CC3)c2c1]

### 4-(1-(3-Oxo-3-(4-(5-(trifluoromethyl)pyrimidin-2-yl)piperazin-1-yl)propoxy)ethyl)-6-(prop-1-ynyl)phthalazin-1(2H)-one

A mixture of 6-bromo-4-(1-(3-oxo-3-(4-(5-(trifluoromethyl)pyrimidin-2-yl)piperazin-1-yl)propoxy)ethyl)phthalazin-1(2H)-one (260 mg, 0.47 mmol), tributyl(prop-1-ynyl)stannane (618 mg, 1.87 mmol) and Pd(dppf)Cl₂ (37 mg, 0.05 mmol) in 1,4-dioxane (8 mL) were stirred at 100° C. for 5 hours. The mixture was cooled to rt, diluted with water (20 mL) and extracted with EtOAc (3-20 mL). The combined organic layer was dried over Na₂SO₄, filtered and concentrated in vacuo. The residue was purified by silica gel chromatography (MeOH/DCM=0 to 5%) to give 4-(1-(3-oxo-3-(4-(5-(trifluoromethyl)pyrimidin-2-yl)piperazin-1-yl)propoxy)ethyl)-6-(prop-1-ynyl)phthalazin-1(2H)-one as a white solid (75 mg, 30% yield). LCMS ESI m/z: 515.3 [M+H]⁺. ¹H NMR (400 MHz, MeOD) δ 8.58 (s, 2H), 8.28-8.23 (m, 2H), 7.74 (dd, J=8.3, 1.4 Hz, 1H), 4.90-4.87 (m, 1H), 3.88-3.81 (m, 5H), 3.77-3.70 (m, 1H), 3.67-3.59 (m, 4H), 2.79-2.60 (m, 2H), 2.08 (s, 3H), 1.62 (d, J=6.7 Hz, 3H).

### Synthesis of Example 193: 4-(1-(Methyl(3-oxo-3-(4-(5-(trifluoromethyl)pyrimidin-2-yl)piperazin-1-yl)propyl) amino)ethyl)-6-(prop-1-yn-1-yl)phthalazin-(2H)-one

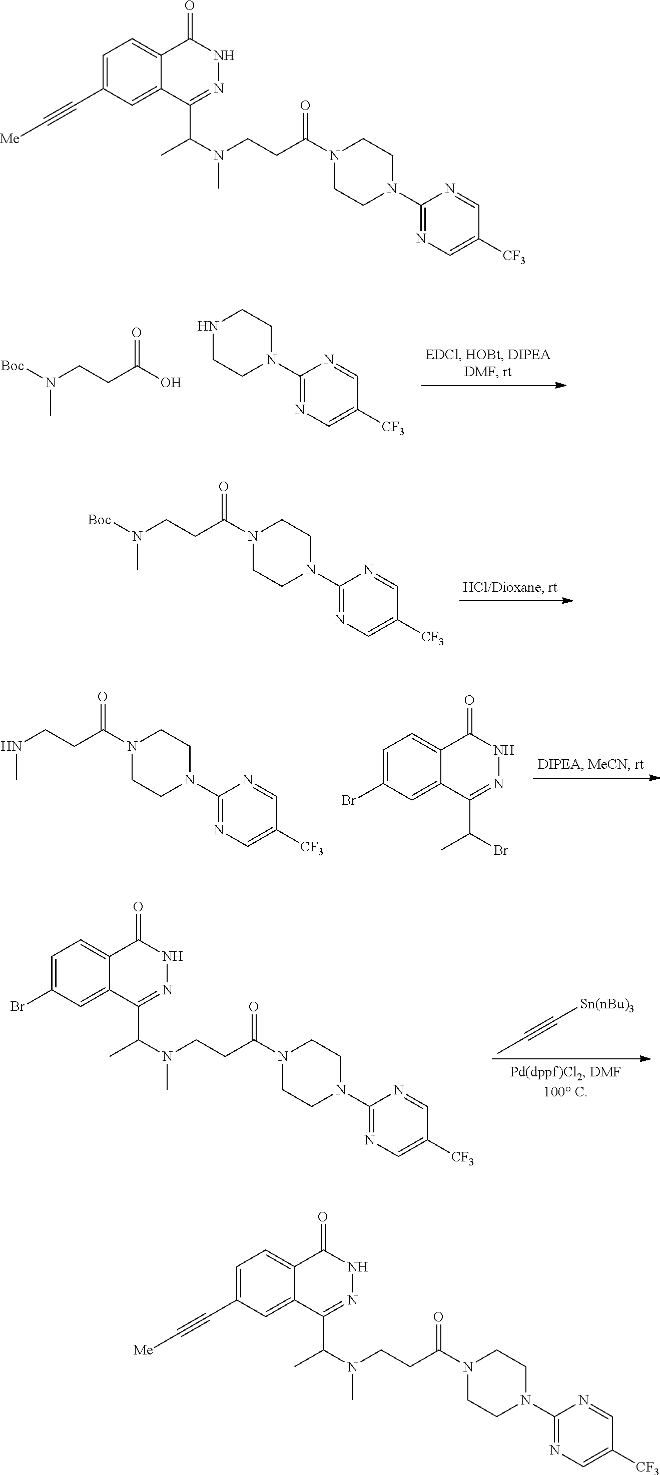

### Step 1

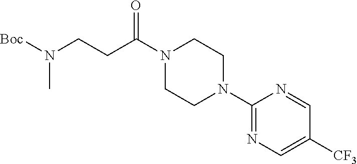 [SMILES: CN(CCC(=O)N1CCN(c2ncc(C(F)(F)F)cn2)CC1)C(=O)OC(C)(C)C]

### tert-Butyl (4-oxo-4-(4-(5-(trifluoromethyl)pyrimidin-2-yl)piperazin-1-yl)butan-2-yl)carbamate

To a solution of 3-((tert-butoxycarbonyl)(methyl)amino)propanoic acid (1.00 g, 4.92 mmol) and 2-(piperazin-1-yl)-5-(trifluoromethyl)pyrimidine (1.32 g, 4.92 mmol) in DMF (10 mL) was added EDCI (1.41 g, 7.38 mmol), HOBt (1.00 g, 7.38 mmol) and DIPEA (1.59 g, 12.30 mmol). The reaction mixture was stirred at rt for 16 hours. The mixture was diluted with H₂O (50 mL) and extracted with EtOAc (50 mL×3). The combined organic layer was washed with brine, dried over anhydrous Na2SO4, filtered and concentrated in vacuo. The residue was purified by silica gel chromatography (EtOAc/petroleum ether=0% to 33%) to give tert-butyl methyl(3-oxo-3-(4-(5-(trifluoromethyl)pyrimidin-2-yl)piperazin-1-yl)propyl)carbamate (1.95 g, 91% yield) as a white solid. LCMS ESI m/z: 418 [M+H]⁺.

### Step 2

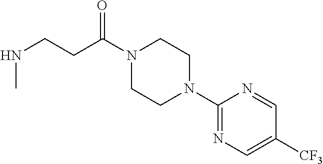 [SMILES: CNCCC(=O)N1CCN(c2ncc(C(F)(F)F)cn2)CC1]

### 3-(Methylamino)-1-(4-(5-(trifluoromethyl)pyrimidin-2-yl)piperazin-1-yl)propan-1-one

A solution of tert-butyl 4-(5-cyanopyridin-2-yl)piperazine-1-carboxylate (1.95 g, 9.58 mmol) in HCl/Dioxane (4M, 5 mL) was stirred at 25° C. for 1 hour. The reaction mixture was concentrated in vacuo to give 3-(methylamino)-1-(4-(5-(trifluoromethyl)pyrimidin-2-yl)piperazin-1-yl)propan-1-one (1.48 g, 99% yield) as a white solid. LCMS ESI m/z: 318 [M+H]⁺. This was used without further purification.

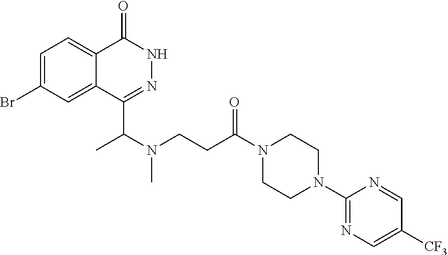 [SMILES: CC(c1n[nH]c(=O)c2ccc(Br)cc12)N(C)CCC(=O)N1CCN(c2ncc(C(F)(F)F)cn2)CC1]

### 6-Bromo-4-(1-(methyl(3-oxo-3-(4-(5-(trifluoromethyl)pyrimidin-2-yl)piperazin-1-yl)propyl)amino)ethyl)phthalazin-1(2H)-one

A solution of 6-bromo-4-(1-bromoethyl)phthalazin-1(2H)-one (113 mg, 0.34 mmol), 3-(methylamino)-1-(4-(5-(trifluoromethyl)pyrimidin-2-yl)piperazin-1-yl)propan-1-one (140 mg, 0.44 mmol) and DIPEA (175 mg, 1.36 mmol) in MeCN (5 mL) was stirred at rt for 2 hours. The reaction was purified by prep-HPLC to give 6-bromo-4-(1-(methyl(3-oxo-3-(4-(5-(trifluoromethyl)pyrimidin-2-yl)piperazin-1-yl)propyl)amino)ethyl)phthalazin-1(2H)-one (183 mg, 95% yield) as a white solid. LCMS ESI m/z: 568 [M+H]⁺.

### Step 4

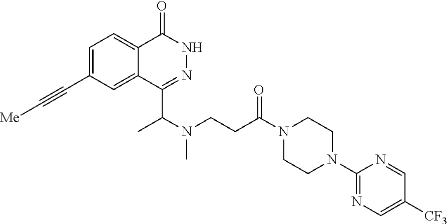 [SMILES: CC#Cc1ccc2c(=O)[nH]nc(C(C)N(C)CCC(=O)N3CCN(c4ncc(C(F)(F)F)cn4)CC3)c2c1]

### 4-(1-(methyl(3-oxo-3-(4-(5-(trifluoromethyl)pyrimidin-2-yl)piperazin-1-yl)propyl)amino)ethyl)-6-(prop-1-yn-1-yl)phthalazin-1(2H)-one

A mixture of tributyl(3,3,3-trifluoroprop-1-ynyl)stannane (260 mg, 0.68 mmol), 6-bromo-4-[1-[methyl-[3-oxo-3-[4-[5-(trifluoromethyl)pyrimidin-2-yl]piperazin-1-yl]propyl]amino]ethyl]-2H-phthalazin-1-one (129 mg, 0.23 mmol) and Pd(dppf)Cl₂ (16 mg, 0.04 mmol) in DMF (4 mL) was stirred at 100° C. under Ar (g) for 19 hours. The reaction mixture was concentrated in vacuo and the residue was purified by prep-HPLC to afford 4-(1-(methyl(3-oxo-3-(4-(5-(trifluoromethyl)pyrimidin-2-yl)piperazin-1-yl)propyl) amino)ethyl)-6-(prop-1-yn-1-yl)phthalazin-1(2H)-one (42 mg, 35% yield) as a white solid. LCMS ESI m/z: 528 [M+H]⁺. ¹H-NMR (400 MHz, DMSO-d₆) δ 12.58 (s, 1H), 8.73 (s, 2H), 8.18-8.09 (m, 2H), 7.70 (d, J=8.2 Hz, 1H), 4.40-4.37 (m, 1H), 3.82-3.74 (m, 2H), 3.65 (t, J=4.9 Hz, 2H), 3.47 (t, J=4.9 Hz, 2H), 3.39-3.35 (m, 1H), 3.32-3.25 (m, 1H), 2.75-2.66 (m, 2H), 2.43 (t, J=6.7 Hz, 2H), 2.25 (s, 3H), 2.07 (s, 3H), 1.28 (d, J=6.6 Hz, 3H).

### Synthesis of Example 194: 4-(((4-Oxo-4-(4-(5-(trifluoromethyl)pyrimidin-2-yl)piperazin-1-yl)butan2yl)amino)methyl)-6-(prop-1-yn-1-yl)phthalazin-1(2H)-one

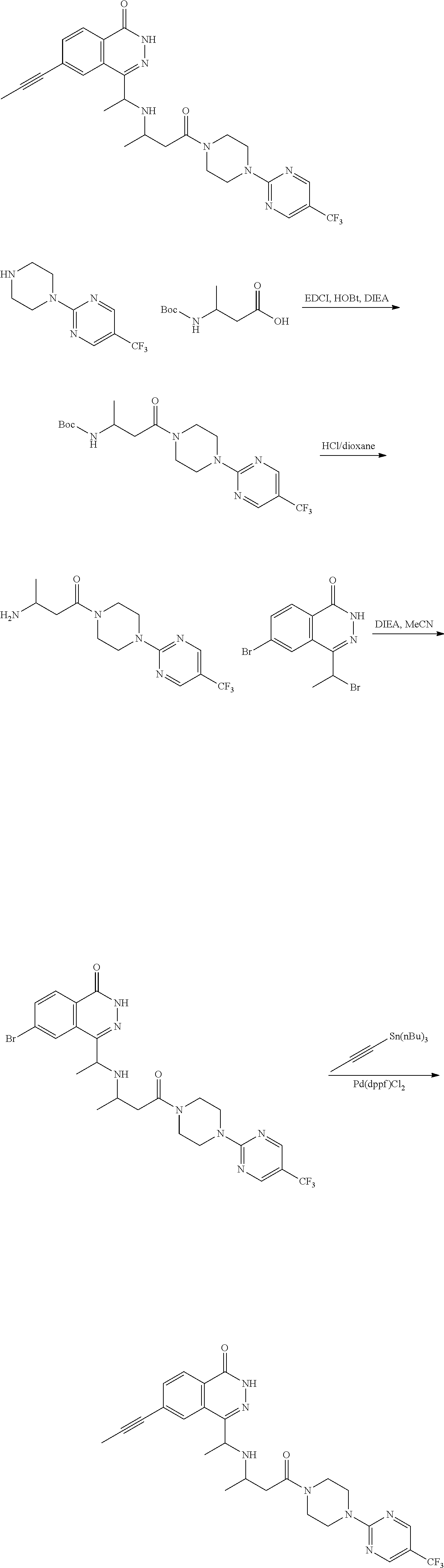

### Step 1

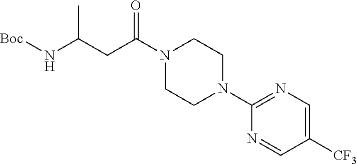 [SMILES: CC(CC(=O)N1CCN(c2ncc(C(F)(F)F)cn2)CC1)NC(=O)OC(C)(C)C]

### tert-Butyl (4-oxo-4-(4-(5-(trifluoromethyl)pyrimidin-2-yl)piperazin-1-yl)butan-2-yl)carbamate

To a solution of 3-((tert-butoxycarbonyl)amino)butanoic acid (2.5 g, 12.30 mmol) and 2-(piperazin-1-yl)-5-(trifluoromethyl)pyrimidine (2.86 g, 10.63 mmol) in DMF (30 mL) was added EDCI (3.54 g, 18.45 mmol). HOBt (2.49 g, 18.45 mmol) and DIPEA (4.77 g, 36.90 mmol). The reaction mixture was stirred at rt for 16 hours. The mixture was diluted with H₂O (50 mL) and extracted with EtOAc (50 mL×3). The combined organic layer was washed with brine, dried over anhydrous Na₂SO₄, filtered and concentrated in vacuo. The residue was purified by silica gel chromatography (EtOAc/petroleum ether=40 to 50%) to give tert-butyl (4-oxo-4-(4-(5-(trifluoromethyl)pyrimidin-2-yl)piperazin-1-yl)butan-2-yl)carbamate as a white solid (4.01 g, 78% yield). LCMS ESI m/z: 418 [M+H]⁺.

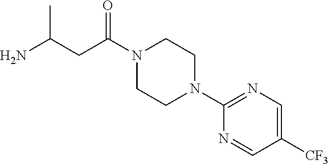 [SMILES: CC(N)CC(=O)N1CCN(c2ncc(C(F)(F)F)cn2)CC1]

### 3-Amino-1-(4-(5-(trifluoro)methyl)pyrimidin-2-yl)piperazin-1-yl)butan-1-one

To a solution of tert-butyl 4-(5-cyanopyridin-2-yl)piperazine-1-carboxylate (4.00 g, 9.58 mmol) in 1,4-dioxane (20 mL) was added HCl/dioxane (4 M, 5 mL), then stirred at 25° C. for 1 hour. The reaction mixture was concentrated in vacuo to give 3-amino-1-(4-(5-(trifluoromethyl)pyrimidin-2-yl)piperazin-1-yl)butan-1-one as a white solid (3.73 g, 99% yield). This was used without further purification. LCMS ESI m/z: 318 [M+H]⁺.

### Step 3

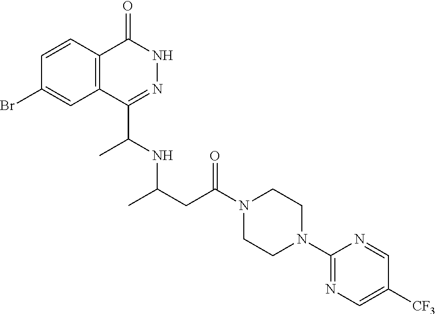 [SMILES: CC(CC(=O)N1CCN(c2ncc(C(F)(F)F)cn2)CC1)NC(C)c1n[nH]c(=O)c2ccc(Br)cc12]

### 6-Bromo-4-(1-((4-oxo-4-(4-(5-(trifluoromethyl)pyrimidin-2-yl)piperazin-1-yl)butan-2-yl)amino)ethyl)phthalazin-1(2H)-one

To a solution of 6-bromo-4-(1-bromoethyl)phthalazin-1(2H)-one (160 mg, 0.48 mmol) and 3-amino-1-(4-(5-(trifluoromethyl)pyrimidin-2-yl)piperazin-1-yl)butan-1-one (199 mg, 0.63 mmol) in MeCN (5 mL) was added DIPEA (249 mg, 1.93 mmol). The reaction mixture was stirred at rt for 2 hours. The mixture was concentrated in vacuo and the residue was purified by prep-HPLC to give 6-bromo-4-(1-((4-oxo-4-(4-(5-(trifluoromethyl)pyrimidin-2-yl)piperazin-1-yl)butan-2-yl)amino)ethyl)phthalazin-1(2H)-one as a white solid (60 mg, 22% yield). LCMS ESI m/z: 568 [M+H]⁺.

### Step 4

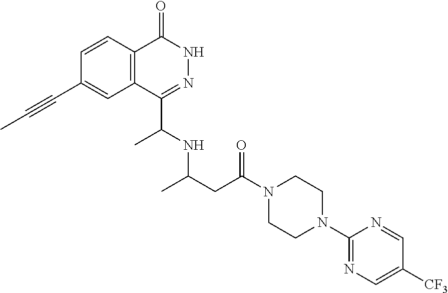 [SMILES: CC#Cc1ccc2c(=O)[nH]nc(C(C)NC(C)CC(=O)N3CCN(c4ncc(C(F)(F)F)cn4)CC3)c2c1]

### 4-(((4-Oxo-4-(4-(5-(trifluoromethyl)pyrimidin-2-yl)piperazin-1-yl)butan-2-yl)amino)methyl)-6-(prop-1-yn-1-yl)phthalazin-1(2H)-one

A mixture of tributyl(prop-1-ynyl)stannane (107 mg, 0.33 mmol), 6-bromo-4-(1-((4-oxo-4-(4-(5-(trifluoromethyl)pyrimidin-2-yl)piperazin-1-yl)butan2yl)amino)ethyl) phthalazin-1-(2H)-one (60 m g, 0.11 mmol) and Pd(dppf)Cl₂ (6 mg, 0.08 mmol) in 1,4-dioxane (6 mL) was stirred at 100° C. under Ar (g) for 2 hours. The mixture was concentrated in vacuo and the residue was purified by prep-HPLC to give 4-(((4-oxo-4-(4-(5-(trifluoromethyl)pyrimidin-2-yl)piperazin-1-yl)butan-2-yl)amino)methyl)-6-(prop-1-yn-1-yl)phthalazin-1(2H)-one as a white solid (35 mg, 63% yield). LCMS ESI m/z: 528.1 [M+H]⁺. ¹HNMR (400 MHz, DMSO-d₆) δ 12.62 (d, J=4.5 Hz, 1H), 8.73 (s, 2H), 8.40 (d, J=13.0 Hz, 1H), 8.17 (d, =9.2 Hz, 1H), 7.80-7.73 (m, 1H), 4.45 (dd, J=21.8, 6.5 Hz, 1H), 3.84-3.67 (m, 4H), 3.53-3.44 (m, 5H), 3.08 (d, J=6.2 Hz, 1H), 2.96 (d, J=6.4 Hz, 1H), 2.37-2.26 (m, 1H), 2.10 (d, J=11.6 Hz, 3H), 1.42-1.34 (m, 3H), 1.06 (dd, J=6.1, 2.6 Hz, 3H).

### Synthesis of Example 195: 4-(6-Oxo-6-(4-(5-(trifluoromethyl)pyrimidin-2-yl)piperazin-1-yl)hexan-2-yl)-6-(prop-1-yn-1-yl)phthalazin-1(2H)-one

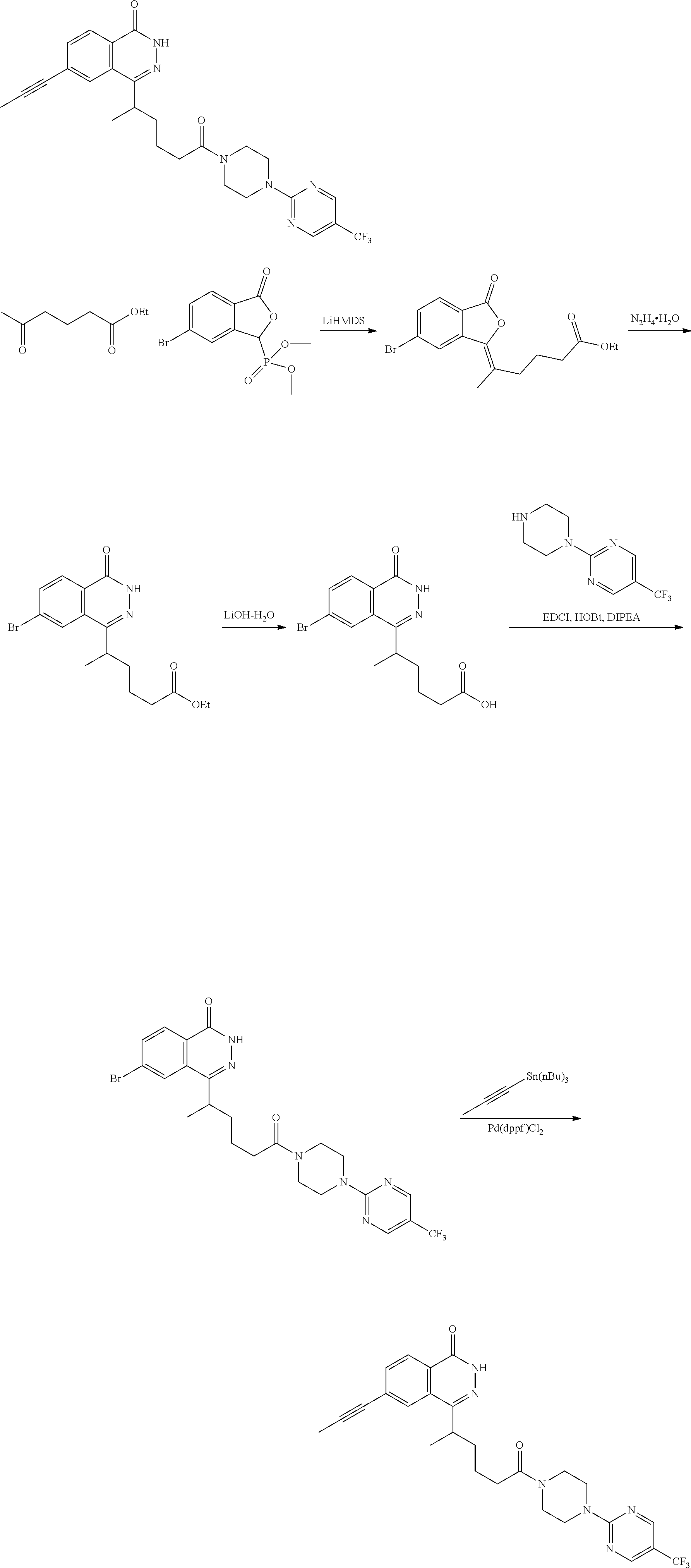

### Step 1

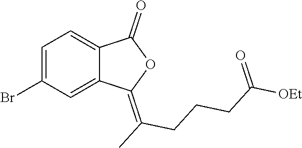 [SMILES: CCOC(=O)CCC/C(C)=C1\OC(=O)c2ccc(Br)cc21]

### Ethyl-5-(6-bromo-3-oxoisobenzofuran-1(3H)-ylidene)hexanoate

To a solution of dimethyl (6-bromo-3-oxo-1,3-dihydroisobenzofuran-1-yl)phosphonate (2.0 g, 6.23 mmol) in THE (18 mL) was added LiHMDS (1.0 M in THF, 6.23 mL, 6.23 mmol) at −78° C., then stirred at −78° C. for 1 hour. Ethyl 5-oxohexanoate (1.18 g, 7.48 mmol) was added to the mixture and the reaction was stirred at rt for 18 hours. The mixture was diluted with H₂O (50 mL) and extracted with DCM (100 mL×3). The combined organic layer was dried over anhydrous Na₂SO₄, filtered and concentrated in vacuo. The residue was purified by silica gel chromatography (EtOAc/petroleum ether=0 to 10%) to afford ethyl 5-(6-bromo-3-oxoisobenzofuran-1(3H)-ylidene)hexanoate as a colorless oil (590 mg, 27% yield). LCMS ESI m/z: 353 [M+H]⁺.

### Step 2

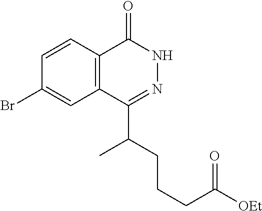 [SMILES: CCOC(=O)CCCC(C)c1n[nH]c(=O)c2ccc(Br)cc12]

### Ethyl 5-(7-bromo-4-oxo-3,4-dihydrophthalazin-1-yl)hexanoate

To a solution of ethyl 5-(6-bromo-3-oxoisobenzofuran-1(3H)-ylidene)hexanoate (590 mg, 1.67 mmol) in THF (10 mL) was added N₂H₄·H₂O (340 mg, 16.70 mmol). The reaction mixture was stirred at 60° C. for 3 hours, then cooled to rt, diluted with H₂O (5 mL) and extracted with EtOAc (10 mL×3). The combined organic layer was washed with brine, dried over anhydrous Na₂SO₄, filtered and concentrated in vacuo. The residue was purified by silica gel chromatography (EtOAc/petroleum ether=10 to 28%) to give ethyl 5-(7-bromo-4-oxo-3,4-dihydrophthalazin-1-yl)hexanoate as a white solid (350 mg, 57% yield). LCMS ESI m/z: 367 [M+H]⁺.

### Step 3

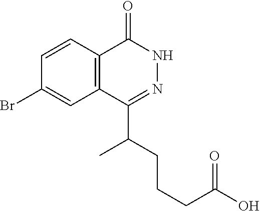 [SMILES: CC(CCCC(=O)O)c1n[nH]c(=O)c2ccc(Br)cc12]

### 5-(7-Bromo-4-oxo-3,4-dihydrophthalazin-1-yl)hexanoic acid

To a solution of methyl 2-bromo-4-cyclopropoxybenzoate (350 mg, 0.95 mmol) in 1,4-dioxane (4 mL) and H₂O (2 mL) was added LiOH—H₂O (400 mg, 9.53 mmol). The mixture was stirred at 25° C. for 3 hours. The organic solvent was removed in vacuo and aq HCl (1N) was added until the solution reached pH 5˜6, then extracted with EtOAc (10 mL×3). The combined organic layer was washed with brine, dried over anhydrous Na₂SO₄, filtered and concentrated in vacuo to afford 2-bromo-4-cyclopropoxybenzoic acid as a white solid (320 mg, 86% yield). This was used without further purification. LCMS ESI m/z: 339 [M+H]⁺.

### Step 4

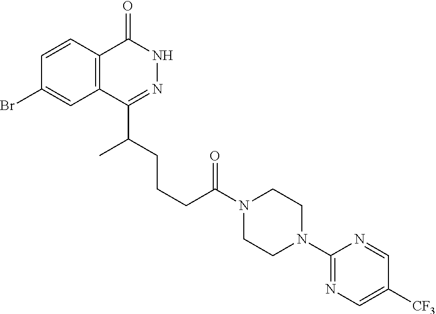 [SMILES: CC(CCCC(=O)N1CCN(c2ncc(C(F)(F)F)cn2)CC1)c1n[nH]c(=O)c2ccc(Br)cc12]

### 6-Bromo-4-(6-oxo-6-(4-(5-(trifluoromethyl)pyrimidin-2-yl)piperazin-1-yl)hexan-2-yl)phthalazin-1(2H)-one

To a solution of 5-(7-bromo-4-oxo-3,4-dihydrophthalazin-1-yl)hexanoic acid (100 mg, 0.29 mmol) and 2-(piperazin-1-yl)-5-(trifluoromethyl)pyrimidine (75 mg, 0.32 mmol) in DMF (5 mL) was added EDCI (85 mg, 0.44 mmol). HOBt (60 mg, 0.44 mmol) and DIPEA (114 mg, 0.88 mmol). The mixture was stirred at rt for 16 hours. The reaction was purified by prep-HPLC to afford 6-bromo-4-(6-oxo-6-(4-(5-(trifluoromethyl) pyrimidin-2-yl)piperazin-1-yl)hexan-2-yl)phthalazin-1(2H)-one as a white solid (80 mg, 49% yield). LCMS ESI m/z: 553 [M+H]⁺.

### Step 5

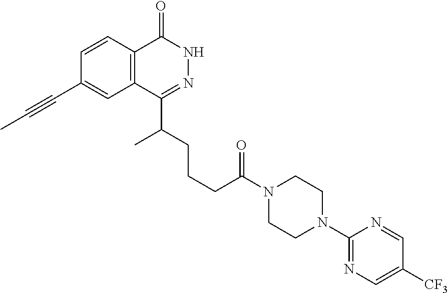 [SMILES: CC#Cc1ccc2c(=O)[nH]nc(C(C)CCCC(=O)N3CCN(c4ncc(C(F)(F)F)cn4)CC3)c2c1]

### 4-(6-Oxo-6-(4-(5-(trifluoromethyl)pyrimidin-2-yl)piperazin-1-yl)hexan-2-yl)-6-(prop-1-yn-1-yl)phthalazin-1(2H)-one

A mixture of 6-bromo-4-(6-oxo-6-(4-(5-(trifluoromethyl)pyrimidin-2-yl)piperazin-1-yl)hexan-2-yl)phthalazin-1(2H)-one (80 mg, 0.14 mmol), tributyl(prop-1-yn-1-yl)stannane (143 mg, 0.42 mmol) and Pd(dppf)Cl₂ (11 mg, 0.01 mmol) in 1,4-dioxane (3 mL) was stirred at 100° C. under Ar (g) for 3 hours. The reaction mixture was concentrated in vacuo. The residue was purified by silica gel chromatography (MeOH/DCM 0-10%) to afford 4-(6-oxo-6-(4-(5-(trifluoromethyl)pyrimidin-2-yl)piperazin-1-yl)hexan-2-yl)-6-(prop-1-yn-1-yl)phthalazin-1(2H)-one (27 mg, 36% yield) as a white solid. LCMS ESI m/z: 513.2 [M+H]⁺. ¹H NMR (400 MHz, DMSO-d₆) δ 12.56 (s, 1H), 8.72 (s, 2H), 8.20 (d, J=8.2 Hz, 1H), 8.03 (s, 1H), 7.78 (d, J=9.2 Hz, 1H), 3.79 (d, J=19.9 Hz, 4H), 3.55-3.50 (m, 4H), 2.37 (t, J=6.7 Hz, 2H), 2.12 (s, 3H), 2.02-1.96 (m, 1H), 1.80 (d, J=6.5 Hz, 1H), 1.54 (d, J=3.3 Hz, 3H), 1.22 (d, J=6.4 Hz, 3H).

### Synthesis of Example 196: 6-(4-(3-(((7-Cyclopropoxy-4-oxo-3,4-dihydrophthalazin-1-yl)methyl)amino)butanoyl)piperazin-1-yl)nicotinonitrile

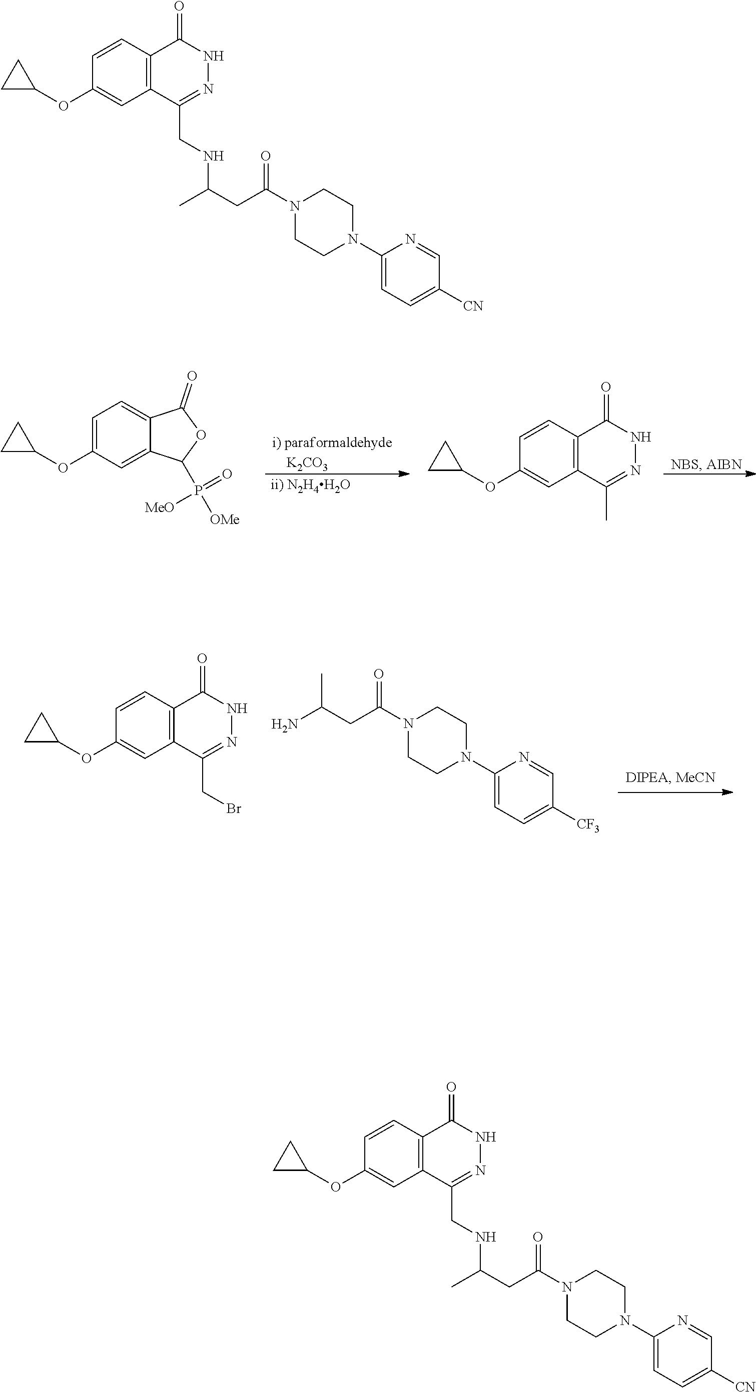 [SMILES: CC(CC(=O)N1CCN(c2ccc(C#N)cn2)CC1)NCc1n[nH]c(=O)c2ccc(OC3CC3)cc12 ; COP(=O)(OC)C1OC(=O)c2ccc(OC3CC3)cc21 ; Cc1n[nH]c(=O)c2ccc(OC3CC3)cc12 ; O=c1[nH]nc(CBr)c2cc(OC3CC3)ccc12 ; CC(N)CC(=O)N1CCN(c2ccc(C(F)(F)F)cn2)CC1 ; CC(CC(=O)N1CCN(c2ccc(C#N)cn2)CC1)NCc1n[nH]c(=O)c2ccc(OC3CC3)cc12]

Step 1: 6-Cyclopropoxy-4-methylphthalazin-1(2H)-one

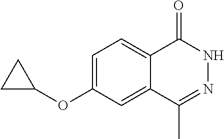 [SMILES: Cc1n[nH]c(=O)c2ccc(OC3CC3)cc12]

A mixture of dimethyl (6-cyclopropoxy-3-oxo-1,3-dihydroisobenzofuran-1-yl)phosphonate (420 mg, 1.41 mmol), paraformaldehyde (423 mg, 14.08 mmol) and K₂CO₃ (389 mg, 2.84 mmol) in anhydrous THF (5 mL) was stirred at 25° C. under Ar (g) for 15 hours. Hydrazine hydrate (1.25 g, 20.03 mmol) was added and the reaction was stirred at 60° C. for 3 hours. THF was removed under vacuum. The formed solids were collected by filtration, washed with water (10 mL) and petroleum ether/EA (2:1, 10 mL) to give 6-cyclopropoxy-4-methylphthalazin-1(2H)-one as a white solid (270 mg, 83% yield). This was used without further purification. LCMS ESI m/z 203 [M+H]⁺.

Step 2: 4-(Bromomethyl)-6-cyclopropoxyphthalazin-(2H)-one

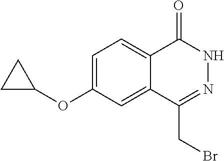 [SMILES: O=c1[nH]nc(CBr)c2cc(OC3CC3)ccc12]

A mixture of 6-cyclopropoxy-4-methylphthalazin-1(2H)-one (225 mg, 1.04 mmol), NBS (445 mg, 2.50 mmol) and AIBN (513 mg, 3.12 mmol) in CCl₄ (8 mL) was stirred at 80° C. for 19 hours. The reaction was quenched with H₂O (10 mL) and extracted with DCM (20 mL×3). The combined organic layer was dried over Na₂SO₄, filtered and concentrated in vacuo. The residue was purified by silica gel chromatography (EtOAc/petroleum ether=30 to 50%) to obtain 4-(bromomethyl)-6-cyclopropoxyphthalazin-1(2H)-one as a yellow oil (145 mg, 32% yield). LCMS ESI m/z 295 [M+H]⁺.

Step 3: 6-(4-(3-(((7-Cyclopropoxy-4-oxo-3,4-dihydrophthalazin-1-yl)methyl)amino) butanoyl)piperazin-1-yl)nicotinonitrile

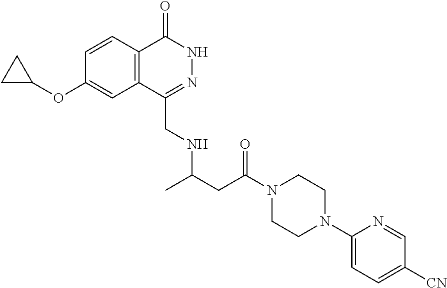 [SMILES: CC(CC(=O)N1CCN(c2ccc(C#N)cn2)CC1)NCc1n[nH]c(=O)c2ccc(OC3CC3)cc12]

A mixture of 4-(bromomethyl)-6-cyclopropoxyphthalazin-1(2H)-one (130 mg, 0.30 mmol), 6-(4-(3-aminobutanoyl)piperazin-1-yl)nicotinonitrile (129 mg, 0.47 mmol) and DIPEA (153 mg, 1.18 mmol) in MeCN (3 mL) was stirred at rt for 16 hours. The mixture was diluted with H₂O (5 mL) and extracted with EtOAc (10 mL×3). The combined organic layer was concentrated in vacuo and the residue was purified by prep-HPLC to give 6-(4-(3-(((7-cyclopropoxy-4-oxo-3,4-dihydrophthalazin-1-yl)methyl)amino) butanoyl)piperazin-1-yl)nicotinonitrile as a white solid (15.6 mg, 11% yield). LCMS ESI m/z 488 (M+H)⁺. ¹H-NMR (4(0) MHz, DMSO-d₆) δ 12.64 (s, 1H), 8.51 (d, J=2.2 Hz, 1H), 8.21 (d, J=8.8 Hz, 1H), 7.89 (dd, J=9.1, 2.3 Hz, 1H), 7.65-7.53 (m, 2H), 6.93 (d, J=9.2 Hz, 1H), 4.36 (d, 2H), 4.15-4.07 (m, 1H), 3.79-3.52 (m, 10H), 2.72 (d, J=37.6 Hz, 2H), 1.28 (d, J=6.1 Hz, 3H), 0.94-0.86 (m, 2H), 0.78-0.71 (m, 2H).

### Synthesis of Example 197: Preparation of N-(2-((5-cyanopyridin-2-yl)(ethyl)amino)ethyl)-2-fluoro-5-((4-oxo-7-(prop-1-yn-1-yl)-3,4-dihydrophthalazin-1-yl)methyl)benzamide

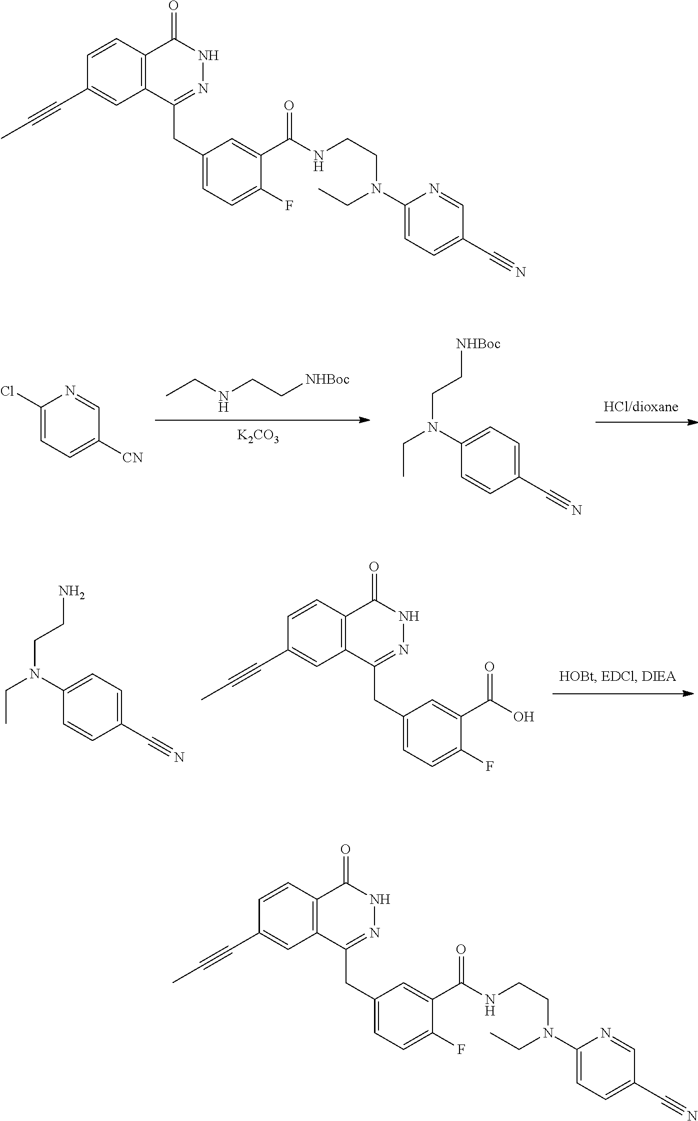

### Step 1

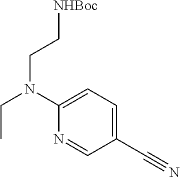 [SMILES: CCN(CCNC(=O)OC(C)(C)C)c1ccc(C#N)cn1]

### tert-Butyl (2-((5-cyanopyridin-2-yl)(ethyl)amino)ethyl)carbamate

A mixture of 6-chloropyridine-3-carbonitrile (810 mg, 5.84 mmol), tert-butyl N-[2-(ethylamino)ethyl]carbamate (1 g, 5.31 mmol) and K₂CO₃ (1.84 g, 13.28 mmol) in MeCN (10 mL) was stirred at 60° C. for 16 hours. The reaction was concentrated in vacuo, diluted with water (30 mL) and extracted with EtOAc (20 mL×3). The combined organic layer was washed with brine, dried over anhydrous Na₂SO₄, filtered and concentrated in vacuo. The residue was purified by silica gel chromatography (EtOAc/petroleum ether=10 to 20%) to give tert-butyl (2-((5-cyanopyridin-2-yl)(ethyl)amino)ethyl)carbamate as a white solid (1.2 g, 70% yield). LCMS ESI m/z: 291 [M+H]⁺.

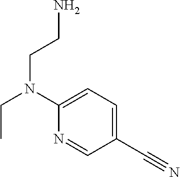

### 6-((2-Aminoethyl)(ethyl)amino)nicotinonitrile

A solution of tert-butyl (2-((5-cyanopyridin-2-yl)(ethyl)amino)ethyl)carbamate (1.2 g, 4.14 mmol) in HCl/dioxane (4 M, 10 mL) was stirred at rt for 1 hour. The solvent was removed in vacuo to give 6-((2-aminoethyl)(ethyl)amino)nicotinonitrile as a white solid (1.12 g, 97% yield). This was used without further purification. LCMS ESI m/z: 191 [M+H]J.

### Step 3

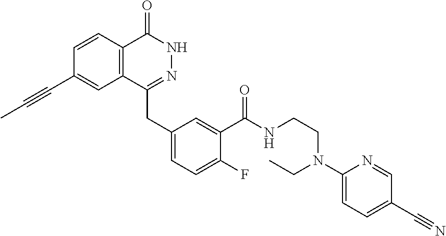 [SMILES: CC#Cc1ccc2c(=O)[nH]nc(Cc3ccc(F)c(C(=O)NCCN(CC)c4ccc(C#N)cn4)c3)c2c1]

### N-(2-((5-Cyanopyridin-2-yl)(ethyl)amino)ethyl)-2-fluoro-5-((4-oxo-7-(prop-1-yn-1-yl)-3,4-dihydrophthalazin-1-yl)methyl)benzamide

To a solution of 2-fluoro-5-((4-oxo-7-(prop-1-yn-1-yl)-3,4-dihydrophthalazin-1-yl)methyl)benzoic acid (100 mg, 0.3 mmol) and 2-fluoro-5-((4-oxo-7-(prop-1-yn-1-yl)-3,4-dihydrophthalazin-1-yl)methyl)benzoic acid (90 mg, 0.3 mmol) in DMF (2 mL) was added EDCI (86 mg, 0.45 mmol). HOBt (60 mg, 0.45 mmol) and DIPEA (192 mg, 1.49 mmol). The reaction mixture was stirred at rt for 1 hour. The reaction was purified by prep-HPLC to give N-(2-((5-cyanopyridin-2-yl)(ethyl)amino)ethyl)-2-fluoro-5-((4-oxo-7-(prop-1-yn-1-yl)-3,4-dihydrophthalazin-1-yl)methyl)benzamide as a white solid (74 mg, 49%). LCMS ESI m/z: 509 [M+H]⁺); ¹H NMR (400 MHz, DMSO-d₆) δ 12.65 (s, 1H), 8.46 (d, J=2.1 Hz, 1H), 8.42 (d, J=2.5 Hz, 1H), 8.20 (d, J=8.2 Hz, 1H), 7.92 (s, 1H), 7.84-7.75 (m, 2H), 7.53-7.44 (m, 2H), 7.23 (dd, J=10.4, 8.5 Hz, 1H), 6.81 (d, J=9.1 Hz, 1H), 4.32 (s, 2H), 3.63 (d, J=5.9 Hz, 2H), 3.55 (d, J=6.9 Hz, 2H), 3.44 (dd, J=12.5, 6.3 Hz, 2H), 2.11 (s, 3H), 1.09 (t, J=7.0 Hz, 3H).

### Synthesis of Example 198: N-(2-((5-Cyanopyridin-2-yl)(ethyl)amino)ethyl)-2-fluoro-N-methyl-5-((4-oxo-7-(prop-1-yn-1-yl)-3,4-dihydrophthalazin-1-yl)methyl)benzamide

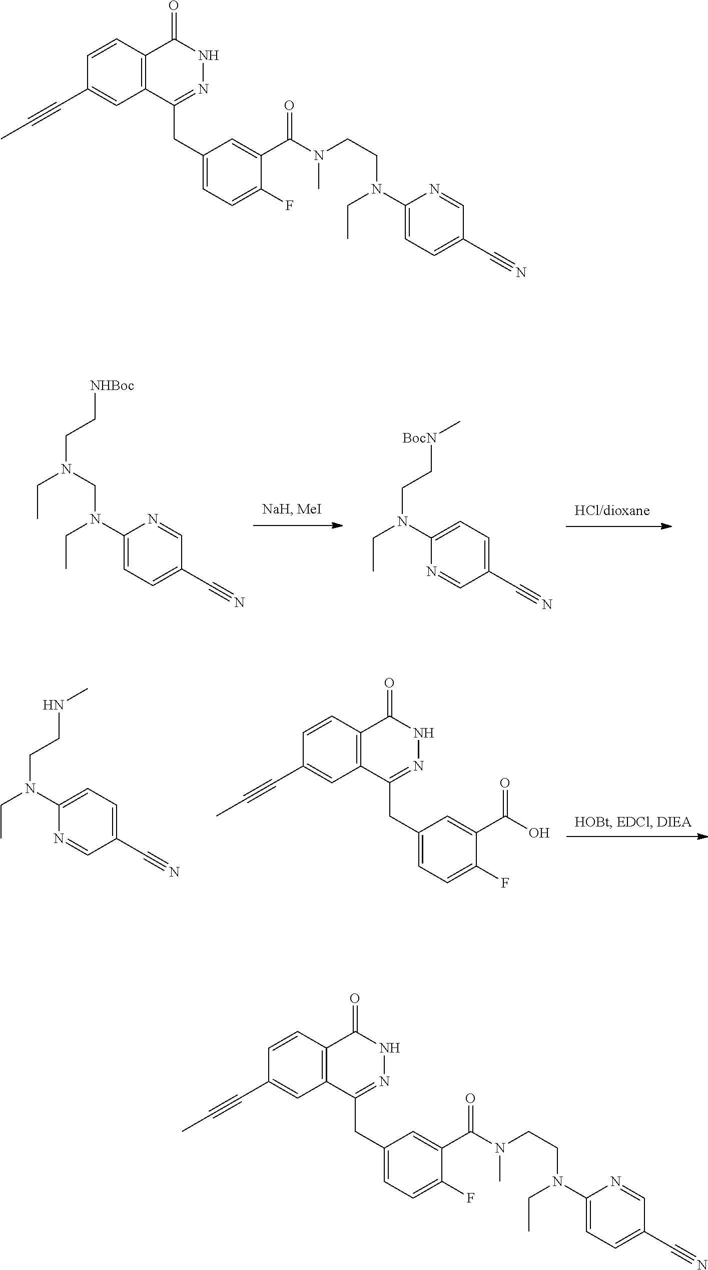 [SMILES: CC#Cc1ccc2c(=O)[nH]nc(Cc3ccc(F)c(C(=O)N(C)CCN(CC)c4ccc(C#N)cn4)c3)c2c1 ; CCN(CCNC(=O)OC(C)(C)C)CN(CC)c1ccc(C#N)cn1 ; CCN(CCN(C)C(=O)OC(C)(C)C)c1ccc(C#N)cn1 ; CCN(CCNC)c1ccc(C#N)cn1 ; CC#Cc1ccc2c(=O)[nH]nc(Cc3ccc(F)c(C(=O)O)c3)c2c1 ; CC#Cc1ccc2c(=O)[nH]nc(Cc3ccc(F)c(C(=O)N(C)CCN(CC)c4ccc(C#N)cn4)c3)c2c1]

### Step 1

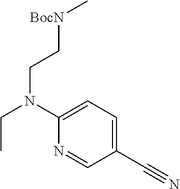 [SMILES: CCN(CCN(C)C(=O)OC(C)(C)C)c1ccc(C#N)cn1]

### tert-Butyl (2-((5-cyanopyridin-2-yl)(ethyl)amino)ethyl)(methyl)carbamate

To a solution of tert-butyl (2-((5-cyanopyridin-2-yl)(ethyl)amino)ethyl)carbamate (300 mg, 1.03 mmol) in DMF (5 mL) was added NaH (60%, 41 mg, 1.03 mmol) at 0° C. under N₂ (g) The mixture was stirred at rt for 1 hour, then CH₃I (170 mg, 1.20 mmol) was added and stirred at rt for 12 hours. The reaction was quenched with water (10 mL) and extracted with EtOAc (20 mL×3). The combined organic layer was washed with brine, dried over anhydrous Na₂SO₄, filtered and concentrated in vacuo. The residue was purified by prep-HPLC to give tert-butyl (2-((5-cyanopyridin-2-yl)(ethyl)amino)ethyl)(methyl) carbamate as a white solid (93 mg, 30% yield). LCMS ESI m/z: 247 [M+H]⁺.

### Step 2

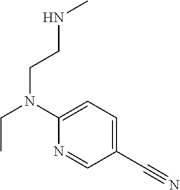

### 6-(Ethyl(2-(methylamino)ethyl)amino)nicotinonitrile

To a solution of tert-butyl (2-((5-cyanopyridin-2-yl)(ethyl)amino)ethyl)(methyl) carbamate (90 mg, 0.30 mmol) in 1,4-dioxane (1 mL) was added HCl/dioxane (4M, 1 mL). The mixture was stirred at rt for 1 hour, then concentrated in vacuo to afford 6-(ethyl(2-(methylamino)ethyl)amino)nicotinonitrile as a white solid (60 mg, 99/o yield). LCMS ESI m/z: 205 [M+H]⁺.

### Step 3

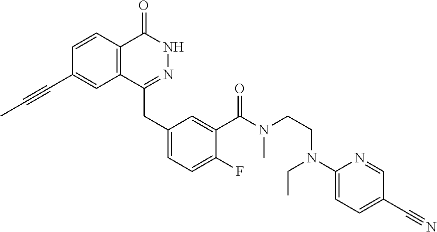 [SMILES: CC#Cc1ccc2c(=O)[nH]nc(Cc3ccc(F)c(C(=O)N(C)CCN(CC)c4ccc(C#N)cn4)c3)c2c1]

### N-(2-((5-Cyanopyridin-2-yl)(ethyl)amino)ethyl)-2-fluoro-N-methyl-5-((4-oxo-7-(prop-1-yn-1-yl)-3,4-dihydrophthalazin-1-yl)methyl)benzamide

To a solution of 6-(ethyl(2-(methylamino)ethyl)amino)nicotinonitrile (60 mg, 0.30 mmol) and 2-fluoro-5-((4-oxo-7-(prop-1-yn-1-yl)-3,4-dihydrophthalazin-1-yl)methyl)benzoic acid (99 mg, 0.30 mmol) in DMF (2 mL) was added EDCI (84 mg, 0.44 mmol), HOBt (60 mg, 0.44 mmol) and DIPEA (114 mg, 0.88 mmol). The reaction mixture was stirred at rt for 2 hours. The reaction was purified by prep-HPLC to afford N-(2-((5-cyanopyridin-2-yl)(ethyl)amino)ethyl)-2-fluoro-N-methyl-5-((4-oxo-7-(prop-1-yn-1-yl)-3,4-dihydrophthalazin-1-yl)methyl)benzamide as a white solid (48 mg, 31% yield). LCMS ESI m/z 523 (M+H)⁺; ¹H NMR (400 MHz, DMSO-d₆) δ 12.48 (s, 1H), 8.45 (s, 0.5H), 8.22-8.14 (m, 1H), 8.03 (s, 0.5H), 7.89-7.84 (m, 1H), 7.82-7.77 (m, 0.5H), 7.74 (d, J=7.9 Hz, 0.5H), 7.69 (d, J=8.2 Hz, 0.5H), 7.54 (d, J=8.8 Hz, 0.5H), 7.38-7.30 (m, 1H), 7.21-7.05 (m, 2H), 6.81 (d, J=9.1 Hz, 0.5H), 6.29 (d, J=8.2 Hz, 0.5H), 4.25 (m, 2H), 3.82-3.74 (m, 1H), 3.69-3.63 (m, 1H), 3.61-3.53 (m, 1H), 3.53-3.46 (m, 1H), 3.40-3.34 (m, 1H), 3.13-3.10 (m, 1H), 3.04 (s, 2H), 2.83 (s, 1H), 2.11-2.06 (m, 3H), 1.14 (t, J=7.0 Hz, 1H), 0.90 (t, J=7.0 Hz, 2H).

### Synthesis of Example 199: 2-Fluoro-5-((4-oxo-7-(prop-1-ynyl)-3,4-dihydrophthalazin-1-yl)methyl)-N-((5-(trifluoromethyl)-1,3,4-oxadiazol-2-yl)methyl)benzamide

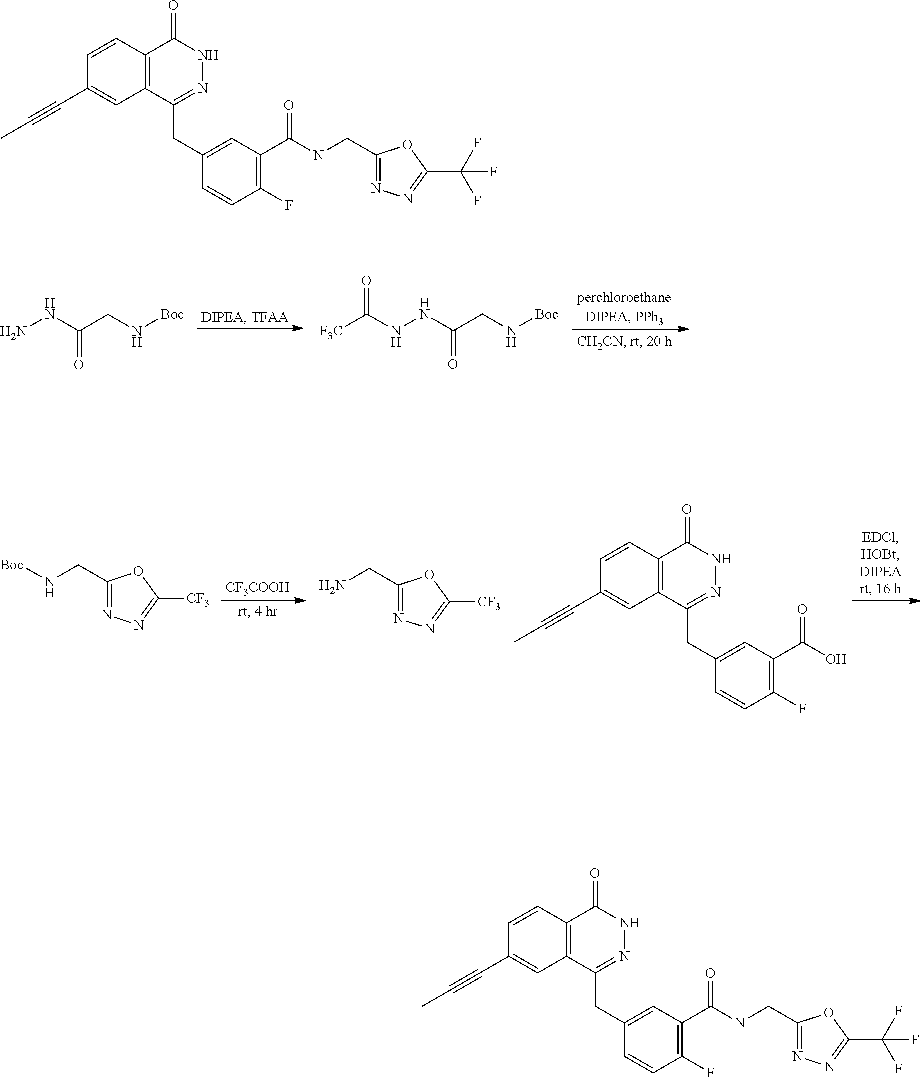

### Step 1

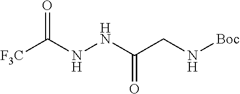 [SMILES: CC(C)(C)OC(=O)NCC(=O)NNC(=O)C(F)(F)F]

### tert-Butyl (2-oxo-2-(2-(2,2,2-trifluoroacetyl)hydrazineyl)ethyl)carbamate

To a solution of tert-butyl (2-hydrazineyl-2-oxoethyl)carbamate (4.8 g, 23.62 mmol) in CH₃CN (100 mL) was added DIPEA (3.66 g, 28.34 mmol, 4.94 mL), cooled to −45° C. 2,2,2-trifluoroacetic anhydride (5.46 g, 25.98 mmol, 3.67 mL) was added dropwise. The mixture was warmed to rt and stirred for 16 hours. The reaction mixture was concentrated to give a residue. The residue was dissolved in water (100 mL), extracted with EtOAc (100 mL×3). The extracts were washed with brine, dried and concentrated to the crude. The crude was purified by silica gel chromatography (petroleum ether/EtOAc=10/1 to 1/1) to give tert-butyl (2-oxo-2-(2-(2,2,2-trifluoroacetyl)hydrazineyl)ethyl)carbamate (6.4 g, 95% yield) as a light yellow gum. ¹H NMR (400 MHz, DMSO-d₆) δ 11.45 (s, 1H), 10.24 (s, 1H), 7.10 (t, J=6.1 Hz, 1H), 3.63 (d, J=6.2 Hz, 2H), 1.38 (s, 9H).

### Step 2

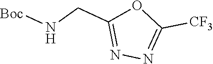 [SMILES: CC(C)(C)OC(=O)NCc1nnc(C(F)(F)F)o1]

### tert-Butyl ((5-(trifluoromethyl)-1,3,4-oxadiazol-2-yl)methyl)carbamate

To a solution of tert-butyl N-[2-oxo-2-[2-(2,2,2-trifluoroacetyl)hydrazino]ethyl]carbamate (500 mg, 1.75 mmol) in CH₃CN (5 mL) was added DIPEA (1.31 g, 10.17 mmol, 1.77 mL) and PPh₃ (1.89 g, 7.19 mmol). The mixture was stirred at 25° C. for 5 minutes, then perchloroethane (954.53 mg, 4.03 mmol, 456.49 μL) was added. The reaction mixture was stirred at rt. for another 20 hours. The mixture was concentrated to the residue. The residue was was worked up with water (100 mL) and EtOAc (350 mL). The organic layer was concentrated to the crude. The crude was purified by silica gel chromatography (EtOAc:petroleum ether=5-15%) to give tert-butyl N-[[5-(trifluoromethyl)-1,3,4-oxadiazol-2-yl]methyl]carbamate (300 mg, 64% yield) as a yellow solid. ¹H-NMR (400 MHz. CDCl₃) δ 5.27 (s, 1H), 4.65 (d, J=5.6 Hz, 2H), 1.46 (s, 9H).

Step 3: (5-(trifluoromethyl)-1,3,4-oxadiazol-2-yl)methanamine

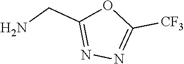 [SMILES: NCc1nnc(C(F)(F)F)o1]

To a solution of tert-butyl N-[[5-(trifluoromethyl)-1,3,4-oxadiazol-2-yl]methyl]carbamate (150 mg, 561.37 μmol) in DCM (2 mL) was added CF₃COOH (1 mL). The solution was stirred at rt. for 4 hours. The mixture was concentrated to give 5-(trifluoromethyl)-1,3,4-oxadiazol-2-yl]methanamine (160 mg, crude) as a yellow oil.

### Step 4

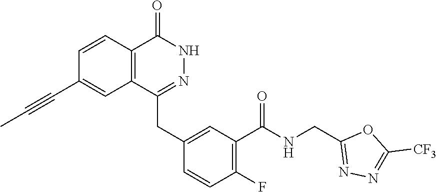 [SMILES: CC#Cc1ccc2c(=O)[nH]nc(Cc3ccc(F)c(C(=O)NCc4nnc(C(F)(F)F)o4)c3)c2c1]

### 2-Fluoro-5-((4-oxo-7-(prop-1-ynyl)-3,4-dihydrophthalazin-1-yl)methyl)-N-((5-(trifluoromethyl)-1,3,4-oxadiazol-2-yl)methyl)benzamide

To a solution of 2-fluoro-5-[(4-oxo-7-prop-1-ynyl-3H-phthalazin-1-yl)methyl]benzoic acid (57.43 mg, 170.75 μmol) in DMF (2 mL) was added DIPEA (294.24 mg, 2.28 mmol, 396.55 μL), EDCI (163.66 mg, 853.75 μmol) and HOBt (115.36 mg, 853.75 μmol). The reaction mixture was stirred at rt for 5 minutes, 5-(trifluoromethyl)-1,3,4-oxadiazol-2-yl]methanamine (160 mg, 569.17 μmol) was added to the mixture. The mixture was stirred at rt for another 16 hours. The mixture was purified by prep-HPLC to give 2-fluoro-5-((4-oxo-7-(prop-1-ynyl)-3,4-dihydrophthalazin-1-yl)methyl)-N-((5-(trifluoromethyl)-1,3,4-oxadiazol-2-yl)methyl)benzamide (3.22 mg, 1.0% yield) as a white solid. LCMS ESI m/z 485.7 [M+H]⁺. ¹H NMR (400 MHz, DMSO-d₆) δ 12.62 (s, 1H), 9.09 (s, 1H), 8.20 (d, J=8.2 Hz, 1H), 7.95 (s, 1H), 7.78 (d, J=8.2 Hz, 1H), 7.62 (dd, J=6.8, 2.0 Hz, 1H4), 7.57-7.48 (m, 1H), 7.29 (dd, J=10.4, 8.6 Hz, 1H), 4.81 (d, J=5.5 Hz, 2H), 4.35 (s, 2H), 2.11 (s, 3H).

### Synthesis of Example 200: 2-Fluoro-5-((4-oxo-7-(prop-1-ynyl)-3,4-dihydrophthalazin-1-yl)methyl)-N-(1-(5-(trifluoromethyl)-1,3,4-oxadiazol-2-yl)ethyl)benzamide

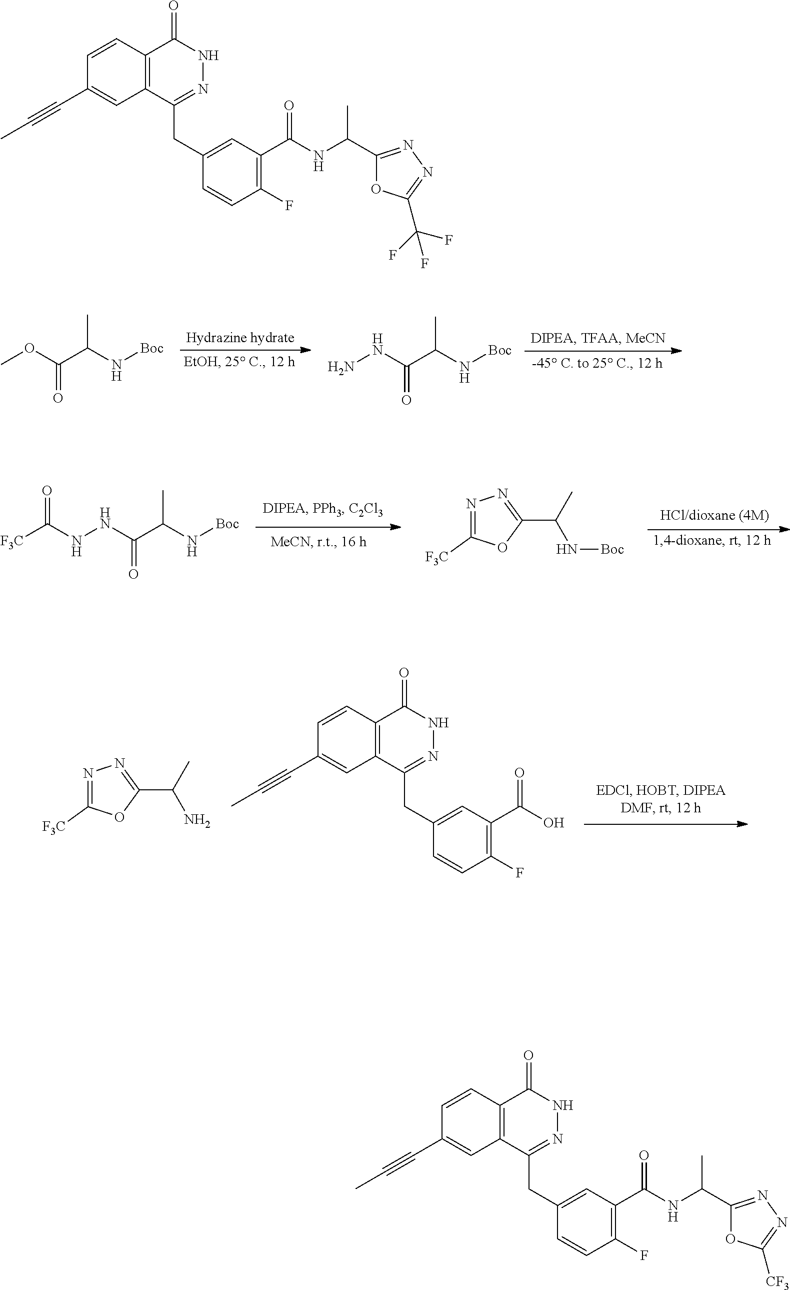

### Step 1

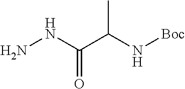

### tert-Butyl 1-hydrazinyl-1-oxopropan-2-ylcarbamate

To a solution of methyl (2S)-2-(tert-butoxycarbonylamino)propanoate (5 g, 24.60 mmol) in Ethanol (140 mL) was added hydrazine hydrate (19.00 g, 379.63 mmol, 18.50 mL), The reaction mixture was stirred at 25° C. for 12 hrs. The reaction mixture was concentrated under reduced pressure. The residue was diluted with H₂O (30 mL) and extracted with DCM (50 mL×2). The combined organic layers were washed with brine (30 ml), dried over Na₂SO₄ and concentrated to afford tert-butyl 1-hydrazinyl-1-oxopropan-2-ylcarbamate (3.7 g, 74.0% yield) as a white solid. ¹H NMR (400 MHz, DMSO-d₆) δ 8.97 (s, 1H), 6.82 (d, J=7.6 Hz, 1H), 4.17 (s, 2H), 3.97-3.85 (m, 1H), 1.37 (s, 9H), 1.14 (d, J=7.1 Hz, 3H)

### Step 2

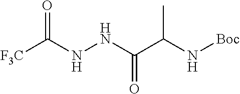 [SMILES: CC(NC(=O)OC(C)(C)C)C(=O)NNC(=O)C(F)(F)F]

### tert-Butyl 1-oxo-1-(2-(2,2,2-trifluoroacetyl)hydrazinyl)propan-2-ylcarbamate

To a solution of tert-butyl N-[(1S)-2-hydrazino-1-methyl-2-oxo-ethyl]carbamate (3.7 g, 18.21 mmol) and DIPEA (2.82 g, 21.85 mmol, 3.81 mL) in CH₃CN (80 mL) at −45° C. under N₂ was added TFAA (4.21 g, 20.03 mmol, 2.83 mL). The reaction was gradually warmed to RT and further stirred for 12 hours. The solvent was removed by evaporation and the residue partitioned between H₂O (25 mL) and EtOAc (25 mL). The organic phase was separated and the aqueous phase was re-extracted with EtOAc (25 mL). The combined organic phases were washed with brine (30 mL), dried over MgSO4 and evaporated, and the residue was purified by silica gel chromatography to afford tert-butyl 1-oxo-1-(2-(2,2,2-trifluoroacetyl)hydrazinyl)propan-2-ylcarbamate (4 g, 73% yield) as a yellow oil. LCMS ESI m/z: 200.1 [M+H]⁺.

### Step 3

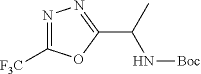 [SMILES: CC(NC(=O)OC(C)(C)C)c1nnc(C(F)(F)F)o1]

### tert-butyl 1-(5-(trifluoromethyl)-1,3,4-oxadiazol-2-yl)ethylcarbamate

To a mixture of tert-butyl N-[1-methyl-2-oxo-2-[2-(2,2,2-trifluoroacetyl)hydrazino]ethyl]carbamate (4 g, 13.37 mmol) in CH₃CN (50 mL) was added PPh₃ (14.37 g, 54.80 mmol), DIPEA (10.02 g, 77.53 mmol, 13.50 mL). The reaction mixture was stirred at rt for 5 minutes, then 1,1,1,2,2,2-hexachloroethane (7.28 g, 30.74 mmol, 3.48 mL) was added, and stirred at rt for 16 hr, The mixture was concentrated to the crude. The crude was diluted into EtOAc (100 mL), washed with water, brine, dried and concentrated. The residue was purified by silica gel chromatography (0-100% EtOAc in heptanes) to give tert-butyl N-[1-[5-(trifluoromethyl)-1,3,4-oxadiazol-2-yl]ethyl]carbamate (2.1 g, 56% yield) as a light yellow oil. ¹H NMR (400 MHz, CDCl₃) δ 5.13 (d, J=14.3 Hz, 2H), 1.64 (d, J=7.0 Hz, 3H), 1.44 (s, 9H)

### Step 4

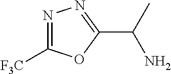

### 1-(5-(trifluoromethyl)-1,3,4-oxadiazol-2-yl)ethanamine

tert-Butyl N-[1-[5-(trifluoromethyl)-1,3,4-oxadiazol-2-yl]ethyl]carbamate (1 g, 3.56 mmol), HCl/dioxane (5 mL) and 1,4-dioxane (10 mL) were added to a 50 mL bottled flask. The resultant mixture was stirred at rt for 12 hr. The solvent removed in vacuo to give 1-(5-(trifluoromethyl)-1,3,4-oxadiazol-2-yl)ethanamine (1 g, crude), which was used to next step without further purification. ¹HNMR (400 MHz, DMSO-d₆) δ 9.18 (s, 2H), 4.95 (d, J=7.0 Hz, 1H), 1.68 (d, J=6.9 Hz, 3H).

### Step 5

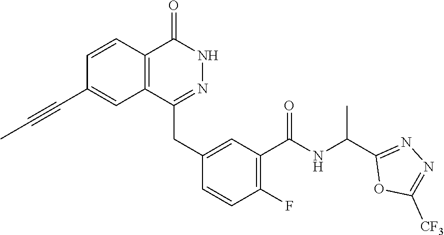 [SMILES: CC#Cc1ccc2c(=O)[nH]nc(Cc3ccc(F)c(C(=O)NC(C)c4nnc(C(F)(F)F)o4)c3)c2c1]

### 2-Fluoro-5-((4-oxo-7-(prop-1-ynyl)-3,4-dihydrophthalazin-1-yl)methyl)-N-(1-(5-(trifluoromethyl)-1,3,4-oxadiazol-2-yl)ethyl)benzamide

Following the general amide coupling procedure in Example 1, but starting with (S)-1-(5-(trifluoromethyl)-1,3,4-oxadiazol-2-yl)ethanamine gave the title compound as a white solid. LCMS ESI m/z: 499.7 [M+H]⁺. ¹H NMR (400 MHz, DMSO-d₆) δ 12.61 (s, 1H), 9.09 (d, J=7.5 Hz, 1H), 8.20 (d, J=8.2 Hz, 1H), 7.94 (s, 1H), 7.77 (dd, J=8.2, 1.3 Hz, 1H), 7.56 (dd, J=6.8, 2.3 Hz, 1H), 7.51-7.46 (m, 1H), 7.26 (dd, J=10.2, 8.6 Hz, 1H), 5.47-5.41 (m, 1H), 4.34 (s, 2H), 2.11 (s, 3H), 1.62 (d, J=7.0 Hz, 3H).

### Synthesis of Example 201: N-(1-(5-Cyanopyridin-2-yl)azetidin-3-yl)-2-fluoro-N-methyl-5-((4-oxo-7-(prop-1-yn-1-yl)-3,4-dihydrophthalazin-1-yl)methyl)benzamide

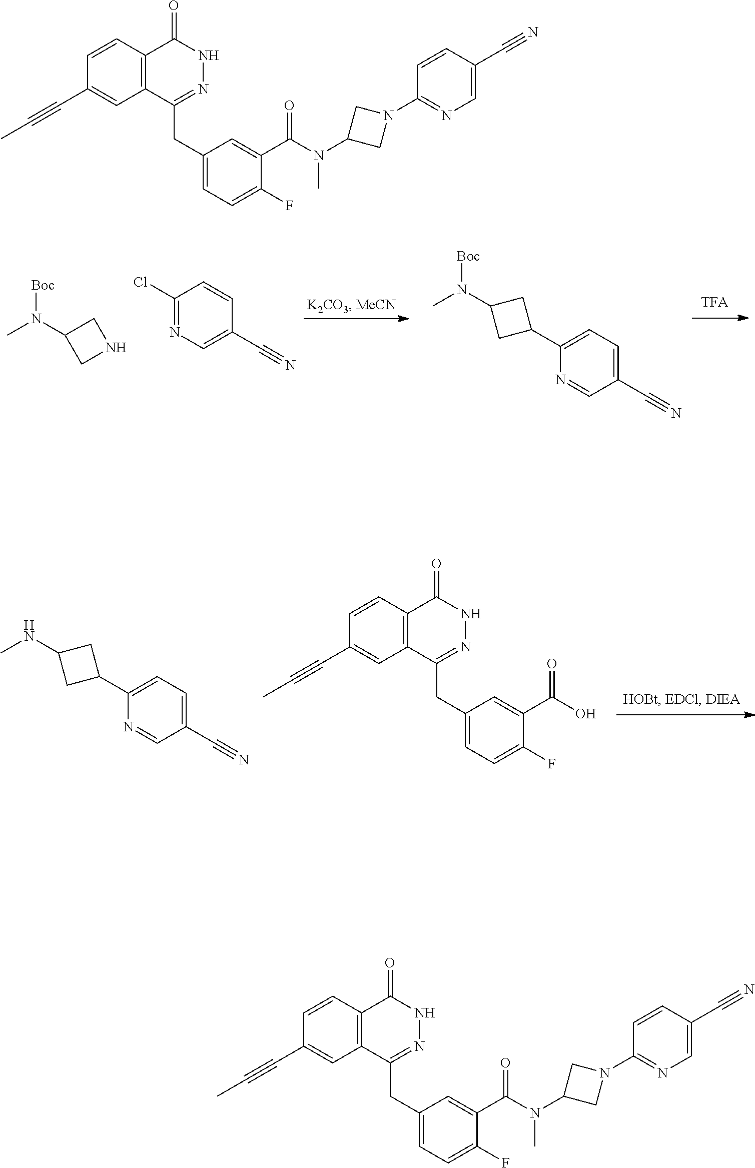

### Step 1

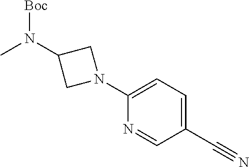 [SMILES: CN(C(=O)OC(C)(C)C)C1CN(c2ccc(C#N)cn2)C1]

### tert-Butyl (1-(5-cyanopyridin-2-yl)azetidin-3-yl)(methyl)carbamate

A mixture of 6-chloropyridine-3-carbonitrile (372 mg, 2.68 mmol), tert-butyl N-(azetidin-3-yl)-N-methyl-carbamate (500 mg, 2.68 mmol) and K₂CO₃ (1.11 g, 8.05 mmol) in MeCN (20 mL) was stirred at 60° C. under Ar (g) for 16 hours. The mixture was cooled to rt, diluted with H₂O (10 mL) and extracted with EtOAc (10 mL×3). The combined organic layer was washed with brine, dried over anhydrous Na₂SO₄, filtered and concentrated in vacuo to give tert-butyl (1-(5-cyanopyridin-2-yl)azetidin-3-yl)(methyl)carbamate as a white solid (0.5 g, 65% yield). This was used without further purification. LCMS ESI m/z 289 [M+H]⁺.

### Step 2

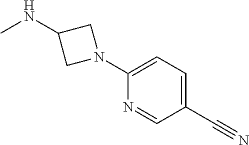

### 6-(3-(Methylamino)azetidin-1-yl)nicotinonitrile

A mixture of tert-butyl N-[1-(5-cyano-2-pyridyl)azetidin-3-yl]-N-methyl-carbamate (500 mg, 1.73 mmol) in DCM (5 mL) and TFA (1 mL) was stirred at 25° C. for 1 hour. The reaction was concentrated in vacuo to give 6-(3-(methylamino)azetidin-1-yl)nicotinonitrile as a brown oil (305 mg, 92% yield). This was used without further purification. LCMS ESI m/z 189 [M+H]⁺.

### Step 3

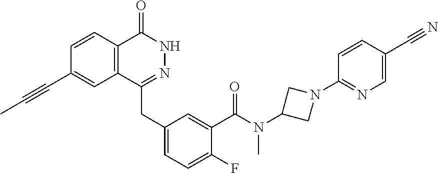 [SMILES: CC#Cc1ccc2c(=O)[nH]nc(Cc3ccc(F)c(C(=O)N(C)C4CN(c5ccc(C#N)cn5)C4)c3)c2c1]

### N-(1-(5-Cyanopyridin-2-yl)azetidin-3-yl)-2-fluoro-N-methyl-5-((4-oxo-7-(prop-1-yn-1-yl)-3,4-dihydrophthalazin-1-yl)methyl)benzamide

To a solution of 2-fluoro-5-[(4-oxo-7-prop-1-ynyl-3H-phthalazin-1-yl)methyl]benzoic acid (50 mg, 0.15 mmol) and 6-[3-(methylamino)azetidin-1-yl]pyridine-3-carbonitrile (37 mg, 0.16 mmol) in DMF (2 mL) was added HOBt (30 mg, 0.23 mmol). EDCI (43 mg, 0.23 mmol) and DIPEA (58 mg, 0.45 mmol). The reaction mixture was stirred at rt for 2 hours and purified by prep-HPLC to give N-(1-(5-cyanopyridin-2-yl)azetidin-3-yl)-2-fluoro-N-methyl-5-((4-oxo-7-(prop-1-yn-1-yl)-3,4-dihydrophthalazin-1-yl)methyl)benzamide as a white solid (38 mg, 50% yield). Yield: 50% LCMS ESI m/z 507 (M+H)⁺; ¹H NMR (400 MHz, DMSO-d₆) δ 12.48 (s, 1H), 8.43 (s, 1H), 8.20 (d, J=8.2 Hz, 1H), 7.90 (s, 1H), 7.80 (d, J=6.7 Hz, 1H), 7.74 (dd, J=8.2, 1.4 Hz, 1H), 7.44-7.26 (m, 2H), 7.22 (t, J=9.1 Hz, 1H), 6.43 (s, 1H), 5.28 (s, 0.5H), 4.56 (s, 0.5H), 4.32 (s, 3H), 4.22-4.16 (m, 2H), 4.15-4.05 (m, 1H), 2.90 (s, 3H), 2.08 (s, 3H).

### Synthesis of Example 202: 2-Fluoro-N-methyl-5-((4-oxo-7-(prop-1-ynyl)-3,4-dihydrophthalazin-1-yl)methyl)-N-((5-(trifluoromethyl)-1,3,4-oxadiazol-2-yl)methyl)benzamide

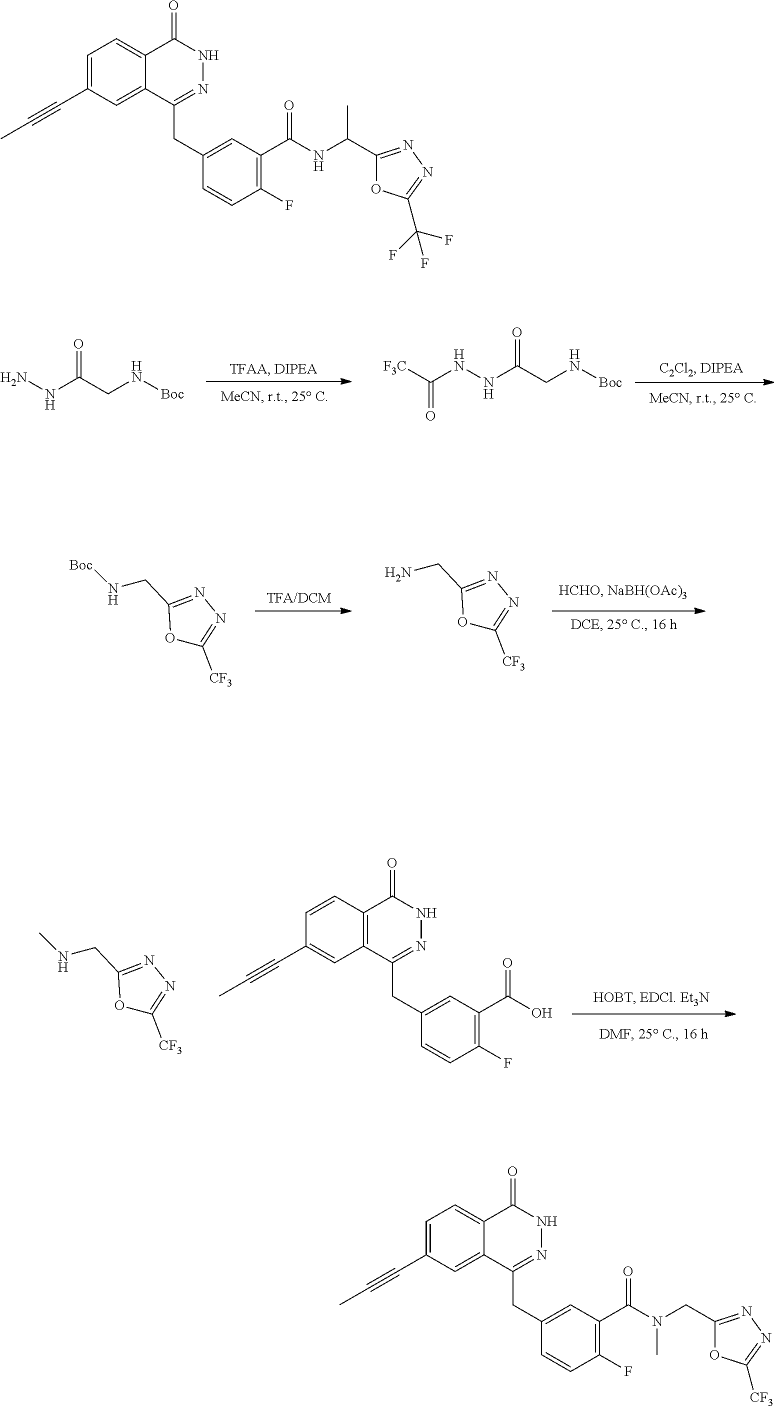

### Step 1

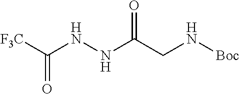 [SMILES: CC(C)(C)OC(=O)NCC(=O)NNC(=O)C(F)(F)F]

### tert-Butyl 2-oxo-2-(2-(2,2,2-trifluoroacetyl)hydrazinyl)ethylcarbamate

To a solution of tert-butyl N-(2-hydrazino-2-oxo-ethyl)carbamate (5.00 g, 26.4 mmol) in CH₃CN (100 mL) was added DIPEA (4.10 g, 31.7 mmol, 5.52 mL), and then cooled to −45° C., then trifluoroacetic anhydride (6.11 g, 29.1 mmol, 4.11 mL) was added dropwise, after the addition, the mixture warmed to rt. slowly, and then stirred at rt. for 16 hrs. The reaction mixture was concentrated. The residue was diluted in H₂O (100 mL), extracted with EA (100 mL×3), washed with brine, dried, concentrated and purified by silica gel chromatography to give tert-butyl N-[2-oxo-2-[2-(2,2,2-trifluoroacetyl)hydrazino]ethyl]carbamate (5.5 g, 73% yield) as a colorless oil. ¹H NMR (400 MHz, DMSO-d₆) δ 11.45 (s, 1H), 10.23 (s, 1H), 7.10 (t, J=6.1 Hz, 1H), 3.62 (t, J=9.7 Hz, 2H), 1.38 (s, 9H).

### Step 2

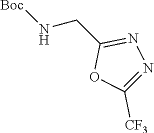 [SMILES: CC(C)(C)OC(=O)NCc1nnc(C(F)(F)F)o1]

### tert-Butyl (5-(trifluoromethyl)-1,3,4-oxadiazol-2-yl)methylcarbamate

To a solution of tert-butyl N-[2-oxo-2-[2-(2,2,2-trifluoroacetyl)hydrazino]ethyl]carbamate (5.50 g, 19.3 mmol) in CH₃CN (100 mL) was added DIPEA (14.5 g, 112 mmol, 19.5 mL) and PPh₃ (20.7 g, 79.1 mmol), and stirred at rt. for 5 minutes, and then perchloroethane (10.5 g, 44.4 mmol, 5.02 mL) was added, and stirred at rt. for 20 hrs. The mixture was concentrated and 50 mL H₂O was added, extracted with EtOAc (100 mL×3), washed with brine, dried, concentrated and purified by silica gel chromatography (EtOAc:petroleum ether=1:10) to give tert-butyl N-[[5-(trifluoromethyl)-1,3,4-oxadiazol-2-yl]methyl]carbamate (3.5 g, 68% yield) as a yellow solid. ¹H NMR (400 MHz, CDCl₃) δ 5.20 (s, 1H), 4.65 (d, J=5.6 Hz, 2H), 1.46 (s, 9H).

### Step 3

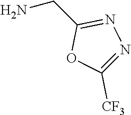 [SMILES: NCc1nnc(C(F)(F)F)o1]

### (5-(Trifluoromethyl)-1,3,4-oxadiazol-2-yl)methanamine

To a solution of tert-butyl N-[[5-(trifluoromethyl)-1,3,4-oxadiazol-2-yl]methyl]carbamate (220 mg, 823 μmol) in DCM (2 mL) was added CF₃COOH (1 mL), and then stirred at rt. for 4 hrs. The reaction mixture was concentrated to give [5-(trifluoromethyl)-1,3,4-oxadiazol-2-yl]methanamine (240 mg, crude) as a light yellow solid.

### Step 4

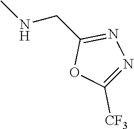

### N-Methyl-1-(5-(trifluoromethyl)-1,3,4-oxadiazol-2-yl)methanamine

A mixture of [5-(trifluoromethyl)-1,3,4-oxadiazol-2-yl]methanamine (150 mg, 898 μmol) and formaldehyde (72.9 mg, 898 μmol, 67 μL) in DCE (9.93 mL) was stirred at 25° C. for 1 hr, sodium triacetoxyborahydride (285 mg, 1.35 mmol) was added to the reaction mixture. Then the reaction mixture was stirred for another 2 hrs. The reaction mixture was acidified with NaHCO₃ (aq.), extracted with EtOAc, the organic layer was concentrated to give N-methyl-1-[5-(trifluoromethyl)-1,3,4-oxadiazol-2-yl]methanamine (100 mg, crude), the crude was used to the next step without further purification. LCMS ESI m/z: 181.9 [M+H]⁺.

### Step 5

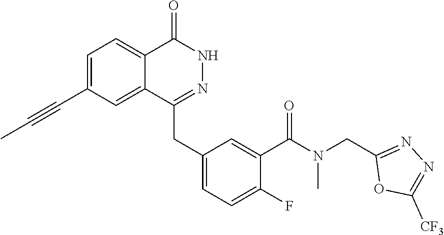 [SMILES: CC#Cc1ccc2c(=O)[nH]nc(Cc3ccc(F)c(C(=O)N(C)Cc4nnc(C(F)(F)F)o4)c3)c2c1]

### 2-Fluoro-N-methyl-5-((4-oxo-7-(prop-1-ynyl)-3,4-dihydrophthalazin-1-yl)methyl)-N-((5-(trifluoromethyl)-1,3,4-oxadiazol-2-yl)methyl)benzamide

Following the general amide coupling procedure in step 2 in Example 1, but starting with N-methyl-1-(5-(trifluoromethyl)-1,3,4-oxadiazol-2-yl)methanamine gave the title compound as a white solid. LCMS ESI m/z: 500.1 [M+H]⁺. ¹H NMR (400 MHz, DMSO-d₆) δ 12.61 (s, 1H), 8.19 (d, J=8.2 Hz, 1H), 7.92-7.91 (m, 1H), 7.76 (dd, J=8.2, 1.3 Hz, 1H), 7.44-7.36 (m, 2H), 7.29-7.22 (m, 1H), 5.06 (s, 2H), 4.35 (s, 2H), 2.97 (s, 3H), 2.10 (s, 3H).

### Synthesis of Example 203: 5-((7-Cyclobutoxy-4-oxo-3,4-dihydrophthalazin-1-yl)methyl)-N-(1-(cyclopropanecarbonyl)azetidin-3-yl)-2-fluorobenzamide

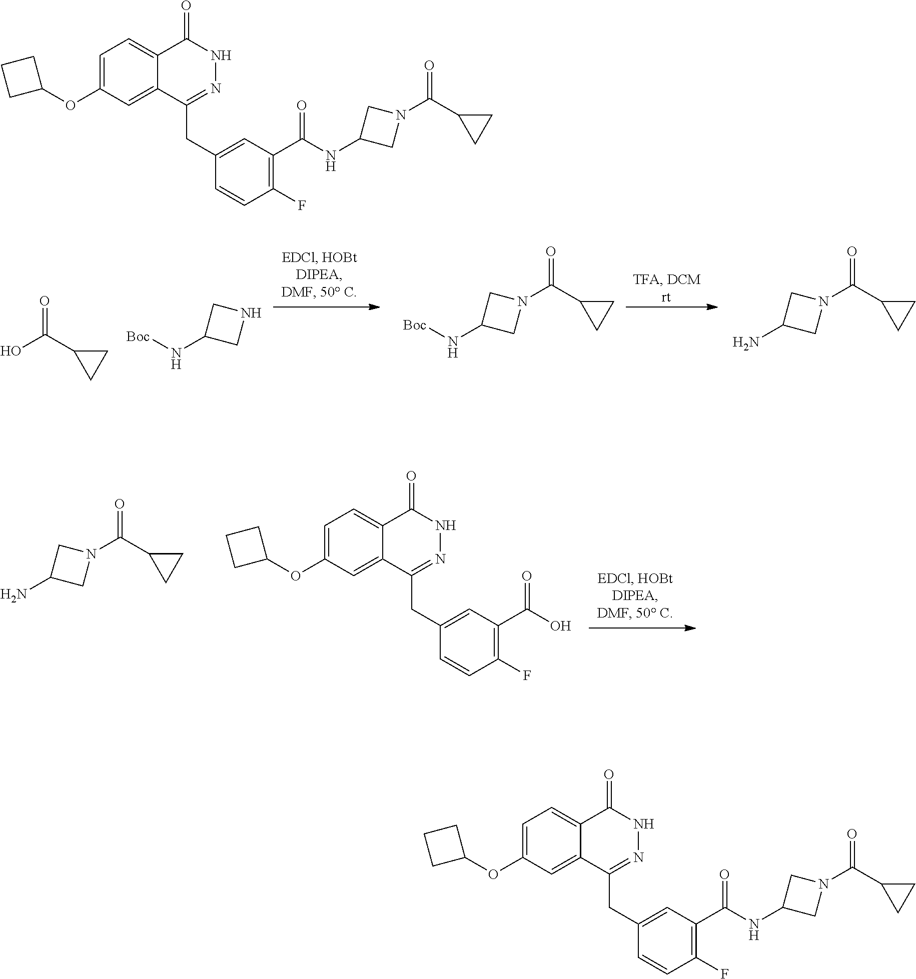

### Step 1

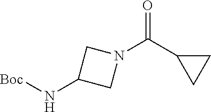 [SMILES: CC(C)(C)OC(=O)NC1CN(C(=O)C2CC2)C1]

### tert-Butyl (1-(cyclopropanecarbonyl)azetidin-3-yl)carbamate

A mixture of cyclopropanecarboxylic acid (300 mg, 3.48 mmol) and tert-butyl azetidin-3-ylcarbamate (600 mg, 3.48 mmol) in DMF (10 mL) was added EDCI (1 g, 5.23 mmol). HOBt (706 mg, 5.23 mmol) and DIPEA (1.35 g, 10.45 mmol). The resulting reaction mixture was stirred at 50 CC for 1 hour, then the reaction was quenched with water (10 mL) and extracted with DCM (30 mL). The organic phase was dried over Na₂SO₄, filtered and concentrated under reduced pressure. The residue was purified by silica gel chromatography (petroleum ether/EtOAc=2/1) to give 6-(4-(3-formylbenzoyl)piperazin-1-yl)nicotinonitrile (560 mg, 67% yield) as a white solid. LCMS ESI m/z: 241.1 [M+H]⁺.

### Step 2

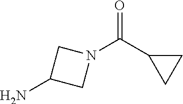

### (3-Aminoazetidin-1-yl)(cyclopropyl)methanone

To a solution of tert-butyl (1-(5-((7-cyclobutoxy-4-oxo-3,4-dihydrophthalazin-1-yl)methyl)-2-fluorobenzoyl)azetidin-3-yl)(methyl)carbamate (560 mg, 2.33 mmol) in DCM (10 mL) was added TFA (1 mL) and stirred at 25° C. for 2 hours. The reaction mixture was concentrated in vacuo to afford (3-aminoazetidin-1-yl)(cyclopropyl)methanone (326 mg, 99% yield) as a colorless oil. This was used without further purification. LCMS ESI m/z: 141.1 [M+H]⁺.

### Step 3

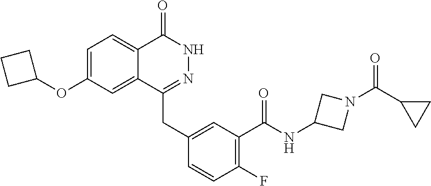 [SMILES: O=C(NC1CN(C(=O)C2CC2)C1)c1cc(Cc2n[nH]c(=O)c3ccc(OC4CCC4)cc23)ccc1F]

### 5-((7-Cyclobutoxy-4-oxo-3,4-dihydrophthalazin-1-yl)methyl)-N-(1-(cyclopropanecarbonyl)azetidin-3-yl)-2-fluorobenzamide

To a solution of (3-aminoazetidin-1-yl)(cyclopropyl)methanone (30 mg, 0.21 mmol) and 5-((7-cyclobutoxy-4-oxo-3,4-dihydrophthalazin-1-yl)methyl)-2-fluorobenzoic acid (79 mg, 0.21 mmol) in DMF (3 mL) was added EDCI (62 mg, 0.32 mmol). HOBt (43 mg, 0.32 mmol) and DIPEA (83 mg, 0.64 mmol). The reaction mixture was stirred at rt for 16 hours. The mixture was diluted with H₂O (10 mL) and extracted with EtOAc (10 mL×3). The combined organic layer was washed with brine, dried over anhydrous Na₂SO₄, filtered and concentrated in vacuo. The residue was purified by prep-HPLC to give 5-((7-cyclobutoxy-4-oxo-3,4-dihydrophthalazin-1-yl)methyl)-N-(1-(cyclopropanecarbonyl) azetidin-3-yl)-2-fluorobenzamide (88.7 mg, 68% yield) as a white solid. LCMS ESI m/z: 491.2 [M+H]⁺. ¹H-NMR (400 MHz, DMSO-d6) δ 12.46 (s, 1H), 8.98 (d, J=6.7 Hz, 1H), 8.14 (d, J=8.8 Hz, 1H), 7.63 (dd, J=6.7, 2.0 Hz, 1H), 7.53-7.40 (m, 1H), 7.34-7.19 (m, 2H), 7.11 (d, J=2.2 Hz, 1H), 4.84 (p, J=7.0 Hz, 1H), 4.74-4.62 (m, 1H), 4.53 (t, J=8.3 Hz, 1H), 4.30 (s, 2H), 4.12 (dd, J=15.7, 7.2 Hz, 2H), 3.80 (dd, J=9.7, 5.3 Hz, 1H), 2.39 (dt, J=9.1, 8.1 Hz, 2H), 2.08-1.90 (m, 2H), 1.76 (dt, J=24.5, 12.4 Hz, 1H), 1.63 (dt, J=18.6, 9.4 Hz, 1H), 1.56-1.47 (m, 1H), 0.69 (d, J=7.7 Hz, 4H).

### Synthesis of Example 204: N-(1-(5-Cyanopyridin-2-yl)azetidin-3-yl)-5-((7-cyclobutoxy-4-oxo-3,4-dihydrophthalazin-1-yl)methyl)-2-fluorobenzamide

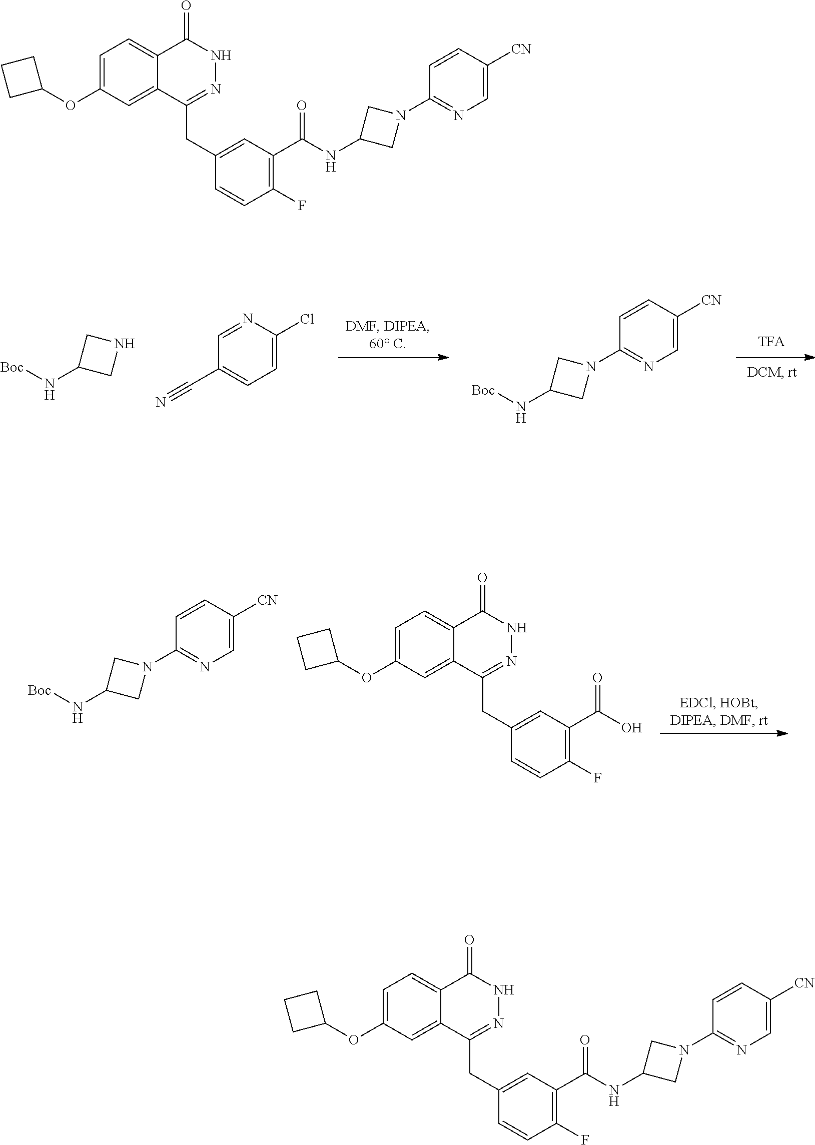

### Step 1

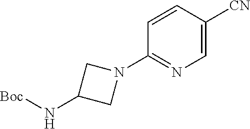 [SMILES: CC(C)(C)OC(=O)NC1CN(c2ccc(C#N)cn2)C1]

### tert-Buty (1-(5-cyanopyridin-2-yl)azetidin-3-yl)carbamate

A mixture of 6-chloronicotinonitrile (241 mg, 1.74 mmol), tert-butyl azetidin-3-ylcarbamate (315 mg, 1.83 mmol) and DIPEA (675 mg, 5.23 mmol) in DMF (6 mL) was stirred at 60° C. for 12 hours. The reaction was diluted with water (20 mL) and extracted with EtOAc (20 mL×3). The combined organic layer was washed with brine, dried over anhydrous Na₂SO₄, filtered and concentrated in vacuo to afford tert-butyl (1-(5-cyanopyridin-2-yl)azetidin-3-yl)carbamate (477 mg, 99% yield) as a off-white solid. LCMS ESI m/z: 275.1 [M+H]⁺.

### Step 2

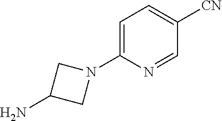 [SMILES: N#Cc1ccc(N2CC(N)C2)nc1]

### 6-(3-Aminoazetidin-1-yl)nicotinonitrile

To a solution of tert-butyl (1-(5-cyanopyridin-2-yl)azetidin-3-yl)carbamate (477 mg, 1.74 mmol) in DCM (10 mL) was added TFA (991 mg, 8.70 mmol). The mixture was stirred at rt for 4 hours. Solvent was removed in vacuo to afford 6-(3-aminoazetidin-1-yl)nicotinonitrile (303 mg, 85% yield) as a white solid. LCMS ESI m/z: 175.1 [M+H]⁺.

### Step 3

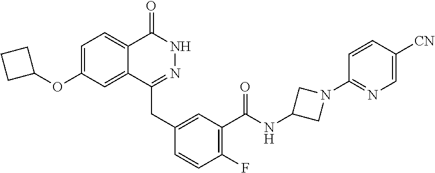 [SMILES: N#Cc1ccc(N2CC(NC(=O)c3cc(Cc4n[nH]c(=O)c5ccc(OC6CCC6)cc45)ccc3F)C2)nc1]

### 5-N-(1-(5-Cyanopyridin-2-yl)azetidin-3-yl)-5-((7-cyclobutoxy-4-oxo-3,4-dihydrophthalazin-1-yl)methyl)-2-fluorobenzamide

To a solution of 6-(3-aminoazetidin-1-yl)nicotinonitrile (40 mg, 0.23 mmol) and 5-((7-cyclobutoxy-4-oxo-3,4-dihydrophthalazin-1-yl)methyl)-2-fluorobenzoic acid (84 mg, 0.23 mmol) in DMF (6 mL) was added EDCI (66 mg, 0.34 mmol), HOBt (46 mg, 0.34 mmol) and DIPEA (118 mg, 0.92 mmol). The reaction mixture was stirred at rt for 4 hours. The reaction was purified by prep-HPLC to give N-(1-(5-cyanopyridin-2-yl)azetidin-3-yl)-5-((7-cyclobutoxy-4-oxo-3,4-dihydrophthalazin-1-yl)methyl)-2-fluorobenzamide (77 mg, 63% yield) as a off-white solid. LCMS ESI m/z: 525.2 [M+H]⁺. ¹H NMR (400 MHz, DMSO-d₆) δ 12.46 (s, 1H), 9.02 (d, J=6.9 Hz, 1H), 8.47 (d, J=1.9 Hz, 1H), 8.15 (d, J=8.8 Hz, 1H), 7.84 (dd, J=8.8, 2.2 Hz, 1H), 7.66-7.61 (m, 1H), 7.50-7.44 (m, 1H), 7.32-7.22 (m, 2H), 7.11 (d, J=2.1 Hz, 1H), 6.48 (d, J=8.8 Hz, 1H), 4.89-4.79 (m, 2H), 4.38 (t, J=8.5 Hz, 2H), 4.30 (s, 2H), 4.00 (dd, J=9.3, 5.4 Hz, 2H), 2.44-2.35 (m, 2H), 2.04-1.95 (m, 2H), 1.83-1.60 (m, 2H).

### Synthesis of Example 205: 5-((7-Cyclobutoxy-4-oxo-3,4-dihydrophthalazin-1-yl)methyl)-2-fluoro-N-(1-(5-(trifluoromethyl)pyrimidin-2-yl)azetidin-3-yl)benzamide

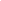

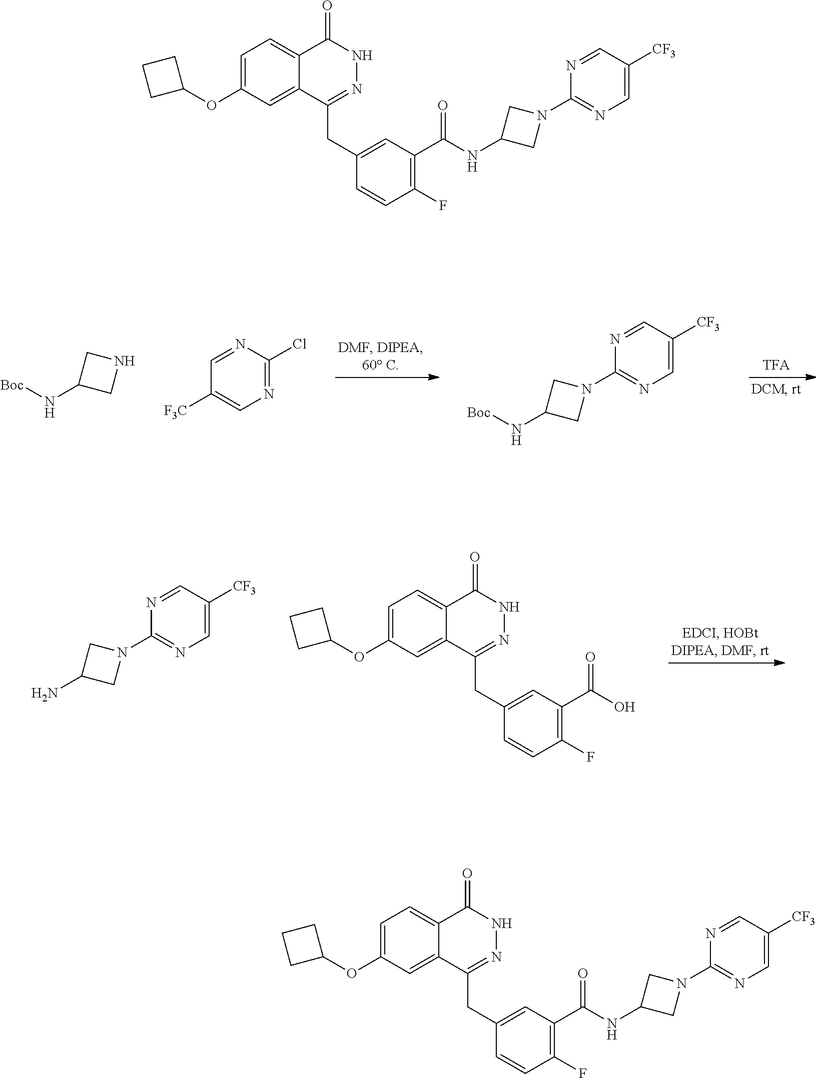

### Step 1

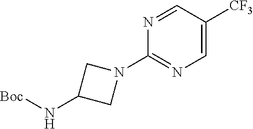 [SMILES: CC(C)(C)OC(=O)NC1CN(c2ncc(C(F)(F)F)cn2)C1]

### tert-Butyl (1-(5-(trifluoromethyl)pyrimidin-2-yl)azetidin-3-yl)carbamate

A mixture of 2-chloro-5-(trifluoromethyl)pyrimidine (183 mg, 1.74 mmol), tert-butyl azetidin-3-ylcarbamate (300 mg, 1.74 mmol) and DIPEA (675 mg, 5.23 mmol) in DMF (10 mL) was stirred at rt for 2 hours. The reaction was diluted with water (20 mL) and extracted with EtOAc (20 mL×3). The combined organic layer was washed with brine, dried over anhydrous Na₂SO₄, filtered and concentrated in vacuo to afford tert-butyl (1-(5-(trifluoromethyl)pyrimidin-2-yl)azetidin-3-yl)carbamate (545 mg, 99% yield) as a white solid. LCMS ESI m/z: 319.1 [M+H]⁺.

### Step 2

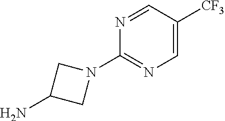 [SMILES: NC1CN(c2ncc(C(F)(F)F)cn2)C1]

### 1-(5-(Trifluoromethyl)pyrimidin-2-yl)azetidin-3-amine

To a solution of tert-butyl (1-(5-(trifluoromethyl)pyrimidin-2-yl)azetidin-3-yl)carbamate (545 mg, 1.71 mmol) in DCM (10 mL) was added TFA (991 mg, 8.70 mmol). The mixture was stirred at rt for 4 hours. The solvent was removed in vacuo to afford 1-(5-(trifluoromethyl)pyrimidin-2-yl)azetidin-3-amine (350 mg, 94% yield) as a white solid. LCMS ESI m/z: 219.1 [M+H]1.

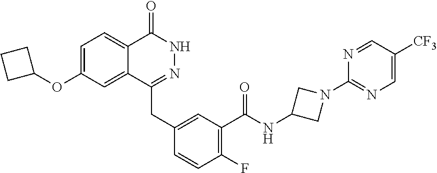 [SMILES: O=C(NC1CN(c2ncc(C(F)(F)F)cn2)C1)c1cc(Cc2n[nH]c(=O)c3ccc(OC4CCC4)cc23)ccc1F]

### 5-((7-Cyclobutoxy-4-oxo-3,4-dihydrophthalazin-1-yl)methyl)-2-fluoro-N-(1-(5-(trifluoromethyl)pyrimidin-2-yl)azetidin-3-yl)benzamide

To a solution of 1-(5-(trifluoromethyl)pyrimidin-2-yl)azetidin-3-amine (50 mg, 0.23 mmol) and 5-((7-cyclobutoxy-4-oxo-3,4-dihydrophthalazin-1-yl)methyl)-2-fluorobenzoic acid (84 mg, 0.23 mmol) in DMF (4 mL) was added EDCI (66 mg, 0.34 mmol). HOBt (46 mg, 0.34 mmol) and DIPEA (118 mg, 0.92 mmol). The reaction mixture was stirred at rt for 4 hours. The reaction was purified by prep-HPLC to give 5-((7-cyclobutoxy-4-oxo-3,4-dihydrophthalazin-1-yl)methyl)-2-fluoro-N-(1-(5-(trifluoromethyl)pyrimidin-2-yl)azetidin-3-yl)benzamide (56 mg, 43% yield) as a white solid. LCMS ESI m/z: 569.2 [M+H]⁺. ¹H-NMR (400 MHz, DMSO-d₆) δ 12.46 (s, 1H), 9.02 (d, J=6.9 Hz, 1H), 8.70 (s, 2H), 8.15 (d, J=8.8 Hz, 1H), 7.69-7.63 (m, 1H), 7.46 (s, 1H), 7.32-7.20 (m, 2H), 7.12 (d, J=2.2 Hz, 1H), 4.89-4.78 (m, 2H), 4.44 (t, J=8.7 Hz, 2H), 4.30 (s, 2H), 4.12-4.05 (m, 2H), 2.45-2.36 (m, 2H), 2.06-1.95 (m, 2H), 1.82-1.59 (m, 2H).

### Synthesis of Example 206: 6-Cyclobutoxy-4-((3-(4-(cyclopropanecarbonyl)piperazin-1-yl)benzo[d]isoxazol-7-yl)methyl)phthalazin-1(2H)-one

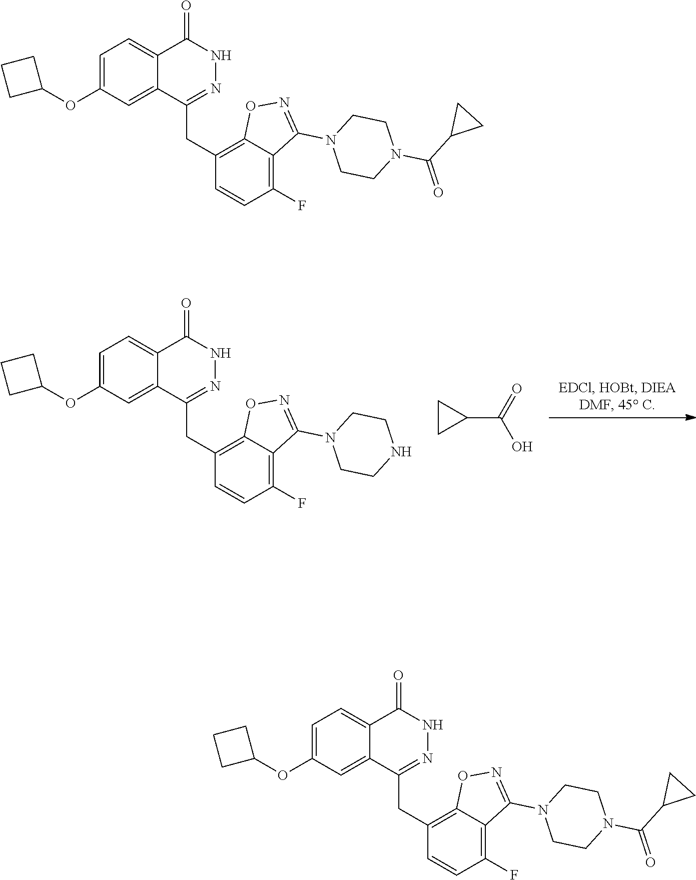

### Step 1

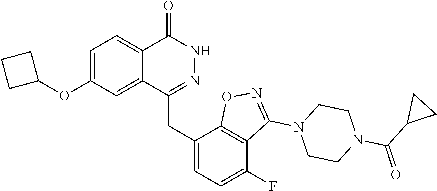 [SMILES: O=C(C1CC1)N1CCN(c2noc3c(Cc4n[nH]c(=O)c5ccc(OC6CCC6)cc45)ccc(F)c23)CC1]

### 6-Cyclobutoxy-4-((3-(4-(cyclopropanecarbonyl)piperazin-1-yl)benzo[d]isoxazol-7-yl)methyl)phthalazin-1(2H)-one

To a solution of 6-cyclobutoxy-4-((3-(piperazin-1-yl)benzo[d]isoxazol-7-yl)methyl)phthalazin-1(2H)-one (108 mg, 0.23 mmol) and cyclopropanecarboxylic acid (20 mg, 0.23 mmol) in DMF (1.5 mL) was added EDCI (66 mg, 0.35 mmol), HOBt (47 mg, 0.35 mmol) and DIPEA (149 mg, 1.15 mmol). The reaction mixture was stirred at 45° C. for 1 hour, then purified by prep-HPLC to afford 6-cyclobutoxy-4-((3-(4-(cyclopropanecarbonyl)piperazin-1-yl)benzo[d]isoxazol-7-yl)methyl)phthalazin-1(2H)-one (40 mg, 35% yield) as a off-white solid. LCMS ESI m/z: 500.1 [M+H]⁺. ¹H NMR (400 MHz, DMSO-d₆) δ 12.43 (s, 1H), 8.15 (d, J=8.8 Hz, 1H), 7.89 (d, J=8.0 Hz, 1H), 7.42 (d, J=7.2 Hz, 1H), 7.32-7.22 (m, 2H), 7.12 (d, J=2.1 Hz, 1H), 4.75-4.66 (m, 1H), 4.50 (s, 2H), 3.88 (s, 2H), 3.67 (s, 2H), 3.56-3.42 (m, 4H), 2.37-2.28 (m, 2H), 2.06-1.92 (m, 3H), 1.83-1.57 (m, 2H), 0.81-0.69 (m, 4H).

### Synthesis of Example 207: 6-Cyclobutoxy-4-((3-(4-(5-(trifluoromethyl)pyrimidin-2-yl)piperazin-1-yl)benzo[d]isoxazol-7-yl)methyl)phthalazin-1(2H)-one

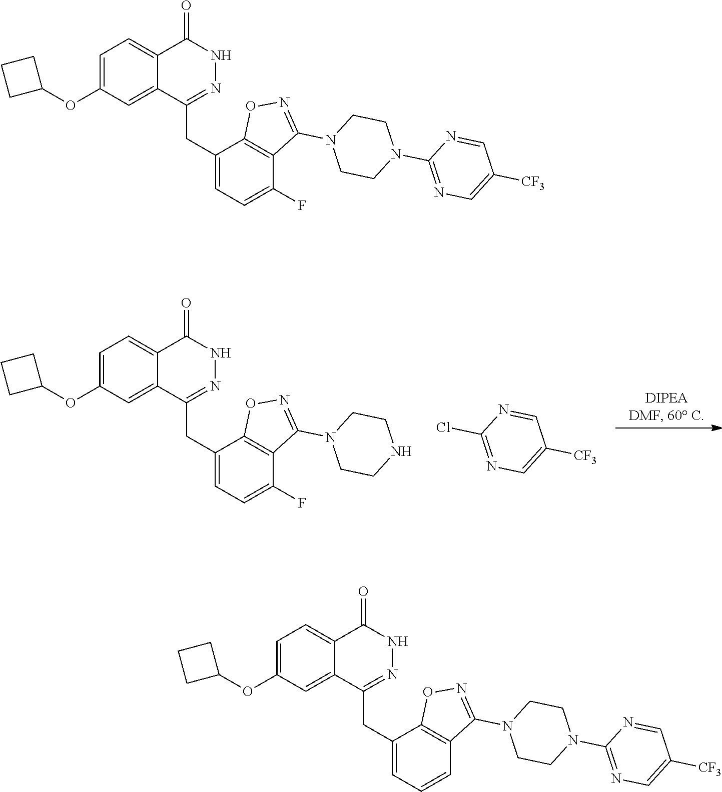 [SMILES: O=c1[nH]nc(Cc2ccc(F)c3c(N4CCN(c5ncc(C(F)(F)F)cn5)CC4)noc23)c2cc(OC3CCC3)ccc12 ; O=c1[nH]nc(Cc2ccc(F)c3c(N4CCNCC4)noc23)c2cc(OC3CCC3)ccc12 ; FC(F)(F)c1cnc(Cl)nc1 ; O=c1[nH]nc(Cc2cccc3c(N4CCN(c5ncc(C(F)(F)F)cn5)CC4)noc23)c2cc(OC3CCC3)ccc12]

### Step 1

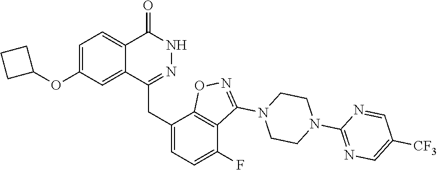 [SMILES: O=c1[nH]nc(Cc2ccc(F)c3c(N4CCN(c5ncc(C(F)(F)F)cn5)CC4)noc23)c2cc(OC3CCC3)ccc12]

### 6-Cyclobutoxy-4-((3-(4-(5-(trifluoromethyl)pyrimidin-2-yl)piperazin-1-yl)benzo[d]isoxazol-7-yl)methyl)phthalazin-1(2H)-one

To a solution of 6-cyclobutoxy-4-((3-(piperazin-1-yl)benzo[d]isoxazol-7-yl)methyl)phthalazin-1(2H)-one (100 mg, 0.23 mmol) and 2-chloro-5-(trifluoromethyl) pyrimidine (46 mg, 0.25 mmol) in DMF (2 mL) was added DIPEA (89 mg, 0.69 mmol). The reaction mixture was stirred at 60° C. for 1 hour. The reaction mixture was purified by prep-HPLC to give 6-(4-(7-((7-cyclobutoxy-4-oxo-3,4-dihydrophthalazin-1-yl)methyl)benzo[d]isoxazol-3-yl)piperazin-1-yl)nicotinonitrile (84 mg, 63% yield) as a white solid. LCMS ESI m/z: 578 (M+H)⁺. ¹H-NMR (400 MHz, DMSO-d₆) δ 12.43 (s, 1H), 8.77 (s, 2H), 8.15 (d, J=8.8 Hz, 1H), 7.93 (d, J=8 Hz, 1H), 7.43 (d, J=7.2 Hz, 1H), 7.32-7.25 (m, 2H), 7.13 (s, 1H), 4.73 (t, J=7.2 Hz, 1H), 4.51 (s, 2H), 4.07-4.04 (m, 4H), 3.62-3.58 (m, 4H), 2.34-2.31 (m, 2H), 2.01-1.61 (m, 4H).

### Synthesis of Example 208: 6-(4-(7-((7-Cyclobutoxy-4-oxo-3,4-dihydrophthalazin-1-yl)methyl)benzo[d]isoxazol-3-yl)piperazin-1-yl)nicotinonitrile

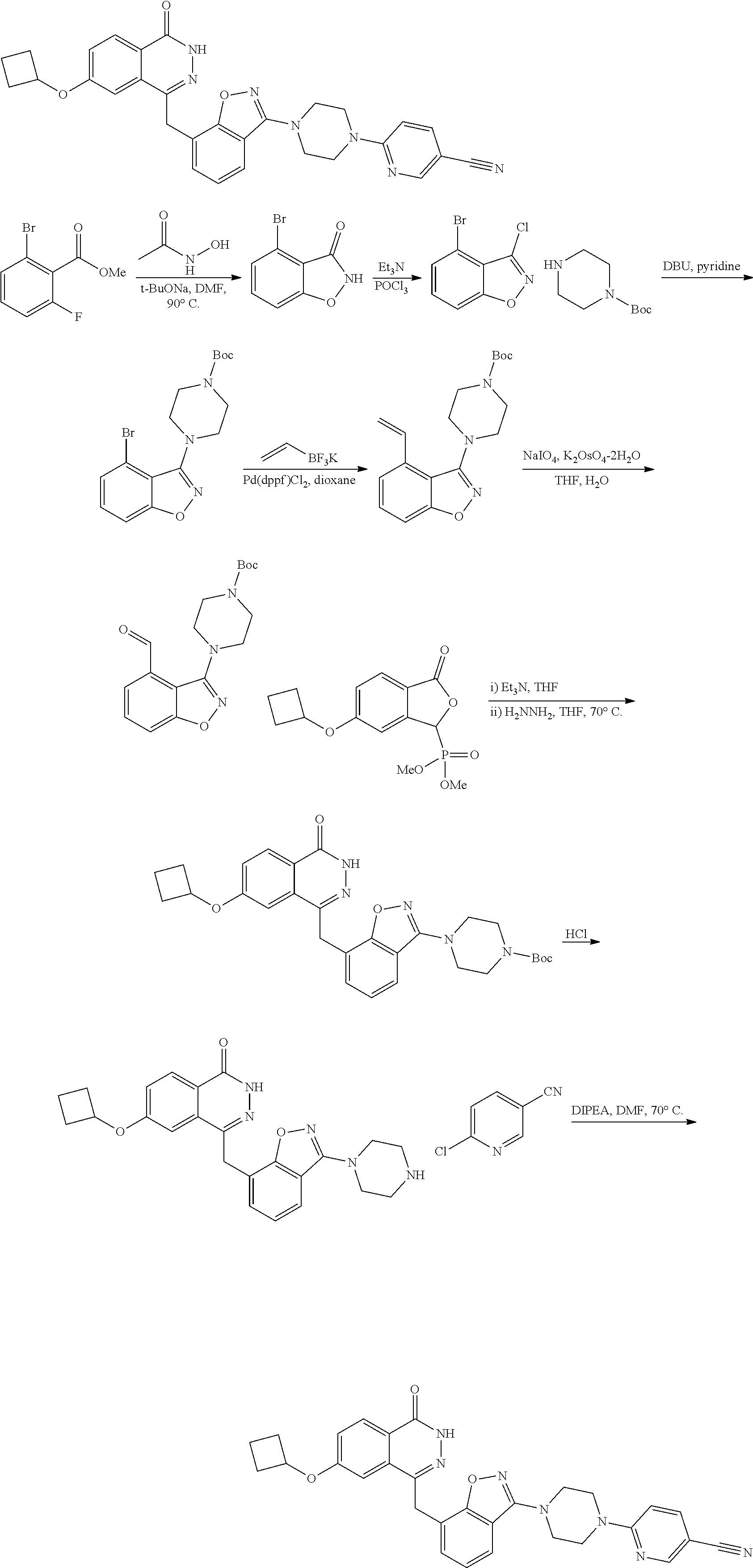

### Step 1

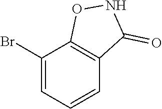 [SMILES: O=c1[nH]oc2c(Br)cccc12]

### 7-bromobenzo[d]isoxazol-3(2H)-one

To a solution of N-hydroxyacetamide (5 g, 66.7 mmol) in DMF (50 mL) was added t-BuONa (7.47 g, 66.7 mmol) and stirred at rt for 30 min. Then ethyl 3-bromo-2-fluorobenzoate (7.73 g, 33.34 mmol) in DMF (20 mL) was added and the reaction was stirred at 90° C. for 4 hours. The mixture was cooled to it, diluted with H₂O (50 mL) and extracted with EtOAc (30 mL×3). The combined organic layer was washed with brine, dried over anhydrous Na₂SO₄, filtered and concentrated in vacuo. The residue was purified by silica gel chromatography (EtOAc/petroleum ether=30 to 50%) to give 7-bromobenzo[d]isoxazol-3(2H)-one (2.55 g, 36% yield) as a white solid. LCMS ESI m/z: 214 [M+H]⁺.

### Step 2

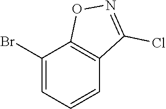 [SMILES: Clc1noc2c(Br)cccc12]

### 7-Bromo-3-chlorobenzo[d]isoxazole

To a mixture of 7-bromobenzo[d]isoxazol-3(2H)-one (2.55 g, 11.92 mmol) in phosphoryl trichloride (20 mL) was added Et₃N (2.41 g, 23.84 mmol). The mixture was stirred at 130° C. for 12 hours. The reaction was carefully diluted with H₂O (50 mL) and extracted with EtOAc (3×30 mL). The combined organic layer was washed with brine, dried over Na₂SO₄, filtered and concentrated in vacuo to give 7-bromo-3-chlorobenzo[d]isoxazole (1.95 g, 71% yield) as a yellow solid. LCMS ESI m/z: 232 [M+H]⁺.

### Step 3

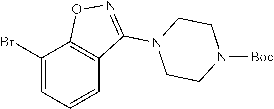 [SMILES: CC(C)(C)OC(=O)N1CCN(c2noc3c(Br)cccc23)CC1]

### tert-butyl 4-(7-bromobenzo[d]isoxazol-3-yl)piperazine-1-carboxylate

A solution of 7-bromo-3-chlorobenzo[d]isoxazole (1.95 g, 8.41 mmol), tert-butyl piperazine-1-carboxylate (4.69 g, 25.23 mmol) and 2,3,4,6,7,8,9,10-octahydropyrimido[1,2-a]azepine (1.92 g, 12.61 mmol) in pyridine (20 mL) was stirred at 110° C. for 12 hours. The reaction was cooled to rt, diluted with water (50 mL) and extracted with EtOAc (50 mL×3). The combined organic layer was washed with brine, dried over Na₂SO₄, filtered and concentrated in vacuo. The residue was purified by silica gel chromatography (EtOAc/petroleum ether=30 to 50%) to give tert-butyl 4-(7-bromobenzo[d]isoxazol-3-yl)piperazine-1-carboxylate (1.88 g, 59% yield) as a white solid. LCMS ESI m/z: 382 [M+H]⁺.

### Step 4

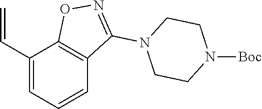 [SMILES: C=Cc1cccc2c(N3CCN(C(=O)OC(C)(C)C)CC3)noc12]

### tert-Butyl 4-(7-vinylbenzo[d]isoxazol-3-yl)piperazine-1-carboxylate

A mixture of tert-butyl 4-(7-bromobenzo[d]isoxazol-3-yl)piperazine-1-carboxylate (1.88 g, 4.92 mmol), potassium vinyltrifluoroborate (0.99 g, 7.38 mmol), Cs₂CO₃ (4.01 g, 12.3 mmol) and Pd(dppf)Cl2 (366 mg, 0.5 mmol) in dioxane (20 mL) was stirred at 80° C. under Ar (g) for 2 hours. The mixture was cooled to rt, diluted with H₂O (20 mL) and extracted with EtOAc (20 mL×3). The combined organic layer was washed with brine, dried over anhydrous Na₂SO₄, filtered and concentrated in vacuo. The residue was purified by silica gel chromatography (EtOAc/petroleum ether=10 to 20%) to give tert-butyl 4-(7-vinylbenzo[d]isoxazol-3-yl)piperazine-1-carboxylate (1.26 g, 78% yield) as a white solid. LCMS ESI m/z: 330 [M+H]⁺.

### Step 5

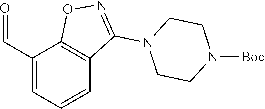 [SMILES: CC(C)(C)OC(=O)N1CCN(c2noc3c(C=O)cccc23)CC1]

### tert-Buty 4-(7-formylbenzo[d]isoxazol-3-yl)piperazine-1-carboxylate

To a mixture of tert-butyl 4-(7-vinylbenzo[d]isoxazol-3-yl)piperazine-1-carboxylate (1.10 g, 3.33 mmol) in THF (40 mL) and H₂O (25 mL) was added K₂OsO₄-2H₂O (33 mg, 0.09 mmol). The mixture was stirred at room temperature for 5 min, then NaIO₄ (2.20 g, 10.19 mmol) was added and the mixture was stirred at rt for 2 hours. The solids were removed by filtration. The filtrate was diluted with H₂O (10 mL) and extracted with EtOAc (30 mL×3). The combined organic layer was washed with brine, dried over anhydrous Na₂SO₄, filtered and concentrated in vacuo to give tert-butyl 4-(7-formylbenzo[d]isoxazol-3-yl)piperazine-1-carboxylate (0.89 g, 81% yield). This was used without further purification. LCMS ESI m/z: 332 [M+H]⁺.

### Step 6

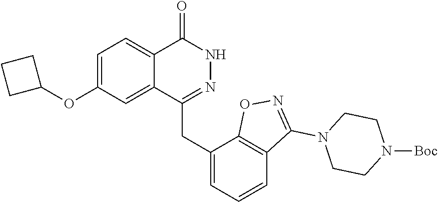 [SMILES: CC(C)(C)OC(=O)N1CCN(c2noc3c(Cc4n[nH]c(=O)c5ccc(OC6CCC6)cc45)cccc23)CC1]

### tert-Butyl 4-(6-((7-cyclobutoxy-4-oxo-3,4-dihydrophthalazin-1-yl)methyl)benzo[d]isoxazol-3-yl)piperazine-1-carboxylate

A solution of tert-butyl 4-(7-formylbenzo[d]isoxazol-3-yl)piperazine-1-carboxylate (450 mg, 1.36 mmol), dimethyl (6-cyclobutoxy-3-oxo-1,3-dihydroisobenzofuran-1-yl)phosphonate (636 mg, 2.04 mmol) and Et₃N (412 mg, 4.07 mmol) in THF (5 mL) was stirred at 60° C. under Ar (g) for 12 hours. Hydrazine hydrate (102 mg, 2.04 mmol) was added and the reaction was heated to 70° C. for 2 hours. The reaction was cooled to rt, and the solvent was removed under vacuum. The formed solids were washed with water (10 mL) and petroleum ether/EtOAc (2:1, 10 mL) to give tert-butyl 4-(7-((7-cyclobutoxy-4-oxo-3,4-dihydrophthalazin-1-yl)methyl)benzo[d]isoxazol-3-yl)piperazine-1-carboxylate (440 mg, 61% yield) as a white solid. LCMS ESI m/z: 532 [M+H]⁺.

### Step 7

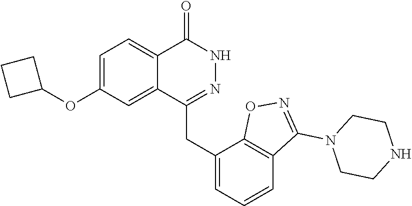 [SMILES: O=c1[nH]nc(Cc2cccc3c(N4CCNCC4)noc23)c2cc(OC3CCC3)ccc12]

### 6-Cyclobutoxy-4-((3-(piperazin-1-yl)benzo[d]isoxazol-7-yl)methyl)phthalazin-1(2H)-one

A solution of tert-butyl 4-(7-((7-cyclobutoxy-4-oxo-3,4-dihydrophthalazin-1-yl)methyl)benzo[d]isoxazol-3-yl)piperazine-1-carboxylate (440 mg, 0.83 mmol) in HCl/dioxane (4 M, 5 mL) was stirred at rt for 1 hour. The solvent was removed in vacuo to give 6-cyclobutoxy-4-((3-(piperazin-1-yl)benzo[d]isoxazol-7-yl)methyl)phthalazin-1(2H)-one (350 mg, 98% yield) as a white solid. This was used without further purification. LCMS ESI 432 [M+H]⁺).

### Step 8

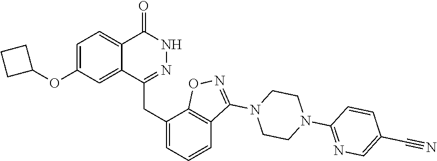 [SMILES: N#Cc1ccc(N2CCN(c3noc4c(Cc5n[nH]c(=O)c6ccc(OC7CCC7)cc56)cccc34)CC2)nc1]

### 6-(4-(7-((7-Cyclobutoxy-4-oxo-3,4-dihydrophthalazin-1-yl)methyl)benzo[d]isoxazol-3-yl)piperazin-1-yl)nicotinonitrile

To a solution of 6-cyclobutoxy-4-((3-(piperazin-1-yl)benzo[d]isoxazol-7-yl)methyl)phthalazin-1(2H)-one (100 mg, 0.23 mmol) and 6-chloronicotinonitrile (35 mg, 0.25 mmol) in DMF (2 mL) was added DIPEA (89 mg, 0.69 mmol). The reaction mixture was stirred at 60° C. for 6 hours. The mixture was purified by prep-HPLC to give 6-(4-(7-((7-cyclobutoxy-4-oxo-3,4-dihydrophthalazin-1-yl)methyl)benzo[d]isoxazol-3-yl)piperazin-1-yl)nicotinonitrile (38 mg, 31% yield) as a white solid. LCMS ESI m/z: 534 [M+H]⁺. ¹H NMR (400 MHz, DMSO-d₆) δ 12.43 (s, 1H), 8.53 (d, J=1.6 Hz, 1H), 8.15 (d, J=8.8 Hz, 1H), 7.93-7.89 (m, 2H), 7.42 (d, J=7.2 Hz, 1H), 7.31-7.24 (m, 2H), 7.12 (d, J=1.2 Hz, 1H), 7.01 (d, J=9.2 Hz, 1H), 4.73 (t, J=7.2 Hz, 1H), 4.50 (s, 2H), 3.90-3.87 (m, 4H), 3.61-3.58 (m, 4H), 2.36-2.30 (m, 2H), 2.00-1.61 (m, 4H).

### Synthesis of Example 209: 6-Cyclobutoxy-4-((3-(4-(5-(trifluoromethyl)pyrimidin-2-yl)piperazin-1-yl)benzo[d]isoxazol-5-yl)methyl)phthalazin-1(2H)-on

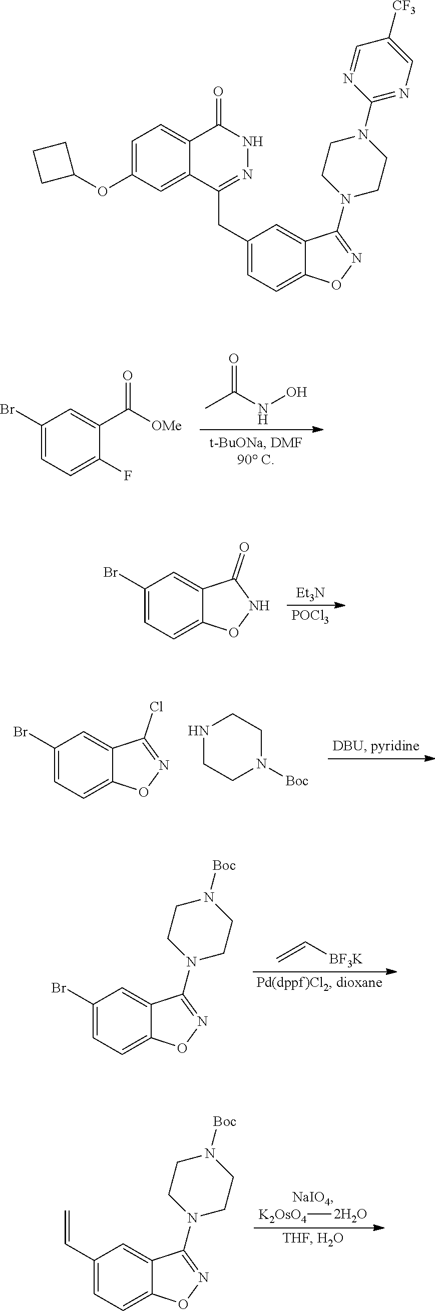

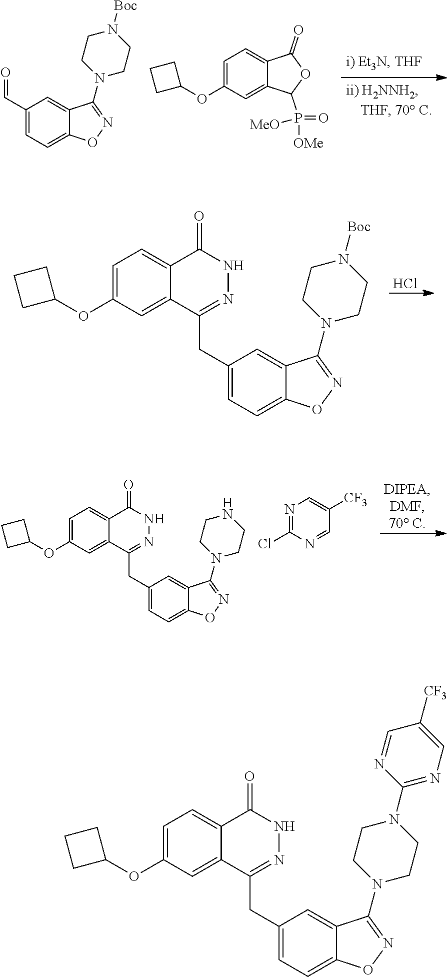

### Step 1

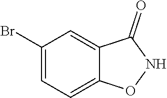 [SMILES: O=c1[nH]oc2ccc(Br)cc12]

### 5-Bromobenzo[d]isoxazol-3(2H)-one

To a solution of N-hydroxyacetamide (3.40 g, 45.30 mmol) in DMF (20 mL) was added t-BuONa (8.70 g, 90.60 mmol) under Ar (g). The reaction was stirred at rt for 40 min, then added methyl 5-bromo-2-fluorobenzoate (5.20 g, 22 mmol). The reaction was stirred at 90° C. for 40 min. then cooled to rt and quenched with sat. NH₄Cl solution (10 mL). The mixture was extracted with EtOAc (30×3 mL). The combined organic layer was washed with bine, dried over Na₂SO₄, filtered and concentrated in vacuo. The residue was purified by silica gel chromatography (EtOAc/petroleum ether=20% to 40%) to give 5-bromobenzo[d]isoxazol-3(2H)-one (2 g 42% yield) as a white solid. LCMS ESI 214 [M+H]⁺.

### Step 2

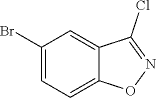 [SMILES: Clc1noc2ccc(Br)cc12]

### 6-Bromo-3-chlorobenzo[d]isoxazole

To a solution of 5-bromobenzo[d]isoxazol-3(2H)-one (2.00 g, 9.38 mmol) in pyridine (1.66 g, 21 mmol) was added POCl₃ (6.45 g, 42 mmol) and Et₃N (1.51 g, 14.95 mmol). The mixture was stirred at 110° C. for 12 hours. The reaction was carefully added to water (10 mL), then extracted with EtOAc (3×30 mL). The combined organic layer was washed with brine, dried over Na₂SO₄, filtered and concentrated in vacuo to give 5-bromo-3-chlorobenzo[d]isoxazole (1.56 g 72% yield) as a white solid. LCMS ESI 232 [M+H]⁺. This was used without further purification.

### Step 3

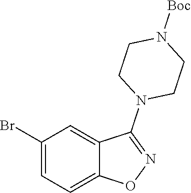 [SMILES: CC(C)(C)OC(=O)N1CCN(c2noc3ccc(Br)cc23)CC1]

### tert-Butyl 4-(5-bromobenzo[d]isoxazol-3-yl)piperazine-1-carboxylate

A mixture of 5-bromo-3-chlorobenzo[d]isoxazole (1.56 g, 6.80 mmol), tert-butyl piperazine-1-carboxylate (1.25 g, 6.70 mmol) and 2,3,4,6,7,8,9,10-octahydropyrimido[1,2-a]azepine (786 mg, 5.16 mmol) in pyridine (20 mL) was stirred at 110° C. for 12 hours. The reaction was diluted with water (50 mL) and extracted with EtOAc (50 mL×3). The combined organic layer was washed with brine, dried over Na2SO₄, filtered and concentrated in vacuo. The residue was purified by silica gel chromatography (EtOAc/petroleum ether=30% to 50%) to give tert-butyl 4-(5-bromobenzo[d]isoxazol-3-yl)piperazine-1-carboxylate (1.20 g 78% yield) as a white solid. LCMS ESI 382 [M+H]1.

### Step 4

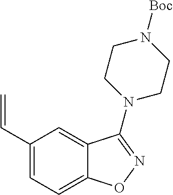 [SMILES: C=Cc1ccc2onc(N3CCN(C(=O)OC(C)(C)C)CC3)c2c1]

### tert-Butyl 4-(5-vinylbenzo[d]isoxazol-3-yl)piperazine-1-carboxylate

A mixture of tert-butyl 4-(5-bromobenzo[d]isoxazol-3-yl)piperazine-1-carboxylate (2.00 g, 5.20 mmol), potassium vinyltrifluoroborate (505 mg, 3.77 mmol). Pd(dppf)Cl₂ (230 mg, 0.31 mmol) and K2CO3 (1.53 g, 11.10 mmol) in dioxane (10 mL) was stirred at 80° C. for 5 hours. The reaction mixture was diluted with H2O (20 mL) and extracted with EtOAc (3×20 mL). The combined organic layer was washed with brine, dried over Na₂SO₄ and filtered. The solvent was removed in vacuo and the residue was purified by silica gel chromatography (EtOAc/petroleum ether=10% to 30%) to give tert-butyl 44(5-vinylbenzo[d]isoxazol-3-yl)piperazine-1-carboxylate (850 mg, 49% yield) as a white solid. LCMS ESI 330 [M+H]⁺.

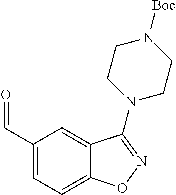 [SMILES: CC(C)(C)OC(=O)N1CCN(c2noc3ccc(C=O)cc23)CC1]

### tert-Buty 4-(5-formylbenzo[d]isoxazol-3-yl)piperazine-1-carboxylate

To a mixture of tert-butyl 4-(5-vinylbenzo[d]isoxazol-3-yl)piperazine-1-carboxylate (850 mg, 2.6 mmol) in THF (36 mL) and H₂O (24 mL) was added K₂OsO₄-2H₂O (27 mg, 0.10 mmol). The mixture was stirred at rt for 5 min, then added NaIO₄ (1.8 g, 4.80 mmol) and stirred at rt for 4 hours. The reaction was diluted with H₂O (20 mL) and extracted with EtOAc (3×20 mL). The combined organic layer was washed with brine, dried over Na₂SO₄ and filtered. The solvent was removed in vacuo to give tert-butyl 4-(5-formylbenzo[d]isoxazol-3-yl)piperazine-1-carboxylate (835 mg 97% yield) as a white solid. This was used without further purification. LCMS ESI 330 [M+H]⁺.

### Step 6

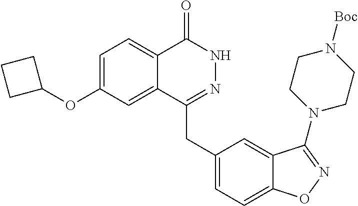 [SMILES: CC(C)(C)OC(=O)N1CCN(c2noc3ccc(Cc4n[nH]c(=O)c5ccc(OC6CCC6)cc45)cc23)CC1]

### tert-Butyl 4-(5-((7-cyclobutoxy-4-oxo-3,4-dihydrophthalazin-1-yl)methyl)benzo[d]isoxazol-3-yl)piperazine-1-carboxylate

A solution of tert-butyl 4-(5-formylbenzo[d]isoxazol-3-yl)piperazine-1-carboxylate (420 mg, 1.27 mmol), dimethyl (6-cyclobutoxy-3-oxo-1,3-dihydroisobenzofuran-1-yl)phosphonate (396 mg, 1.27 mmol) and Et₃N (385 mg, 3.80 mmol) in THF (5 mL) was stirred at 50° C. under Ar (g) for 15 hours. Hydrazine hydrate (102 mg, 2.04 mmol) was added and the reaction was stirred at 70° C. for 1 hour. The reaction was cooled to rt, and the solvent was removed under vacuum. The formed solids were filtrated, washed with water (10 mL) and petroleum ether/EA (2:1, 10 mL) to give tert-butyl 4-(5-((7-cyclobutoxy-4-oxo-3,4-dihydrophthalazin-1-yl)methyl)benzo[d]isoxazol-3-yl)piperazine-1-carboxylate (490 mg 80% yield) as a white solid. LCMS ESI 532 [M+H]⁺.

### Step 7

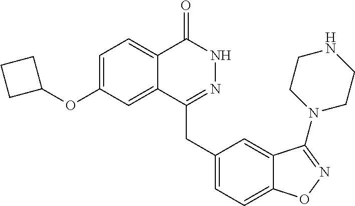 [SMILES: O=c1[nH]nc(Cc2ccc3onc(N4CCNCC4)c3c2)c2cc(OC3CCC3)ccc12]

### 6-cyclobutoxy-4-((3-(piperazin-1-yl)benzo[d]isoxazol-5-yl)methyl)phthalazin-1(2H)-one

A solution of tert-butyl 4-(5-((7-cyclobutoxy-4-oxo-3,4-dihydrophthalazin-1-yl)methyl)benzo[d]isoxazol-3-yl)piperazine-1-carboxylate (490 mg, 0.94 mmol) in HCl/dioxane (4M, 5 mL) was stirred at rt for 1 hour. The solvent was removed in vacuo to give 6-cyclobutoxy-4-((3-(piperazin-1-yl)benzo[d]isoxazol-5-yl)methyl)phthalazin-1(2H)-one (395 mg 99% yield) as a white solid. This was used without further purification. LCMS ESI 432[M+H]⁺.

### Step 8

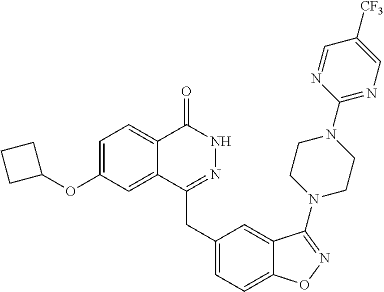 [SMILES: O=c1[nH]nc(Cc2ccc3onc(N4CCN(c5ncc(C(F)(F)F)cn5)CC4)c3c2)c2cc(OC3CCC3)ccc12]

### 6-Cyclobutoxy-4-((3-(4-(5-(trifluoromethyl)pyrimidin-2-yl)piperazin-1-yl)benzo[d]isoxazol-5-yl)methyl)phthalazin-1(2H)-one

To a solution of 6-cyclobutoxy-4-((3-(piperazin-1-yl)benzo[d]isoxazol-5-yl)methyl)phthalazin-1(2H)-one (120 mg, 0.24 mmol) and 2-chloro-5-(trifluoromethyl) pyrimidine (50 mg, 0.28 mmol) in DMF (2 mL) was added DIPEA (112 mg, 0.85 mmol). The reaction mixture was stirred at 60° C. for 2 hours. The mixture was purified by prep-HPLC to give 6-cyclobutoxy-4-((3-(4-(5-(trifluoromethyl)pyrimidin-2-yl)piperazin-1-yl)benzo[d]isoxazol-5-yl)methyl)phthalazin-1(2H)-one (77 mg 60% yield) as a white solid. LCMS ESI 578 [M+H]⁺. ¹H NMR (400 MHz, DMSO-d₆) δ 12.44 (s, 1H), 8.77 (s, 2H), 8.27-8.06 (m, 2H), 7.57-7.42 (m, 2H), 7.33-7.15 (m, 2H), 4.85 (p, J=7.1 Hz, 1H), 4.42 (s, 2H), 4.14-3.99 (m, 4H), 3.68-3.50 (m, 4H), 2.44-2.29 (m, 2H), 2.08-1.91 (m, 2H), 1.84-1.62 (m, 2H).

### Synthesis of Example 210: 6-cyclobutoxy-4-((3-(4-(cyclopropanecarbonyl)piperazin-1-yl)benzo[d]isoxazol-5-yl)methyl)phthalazin-1(2H)-one

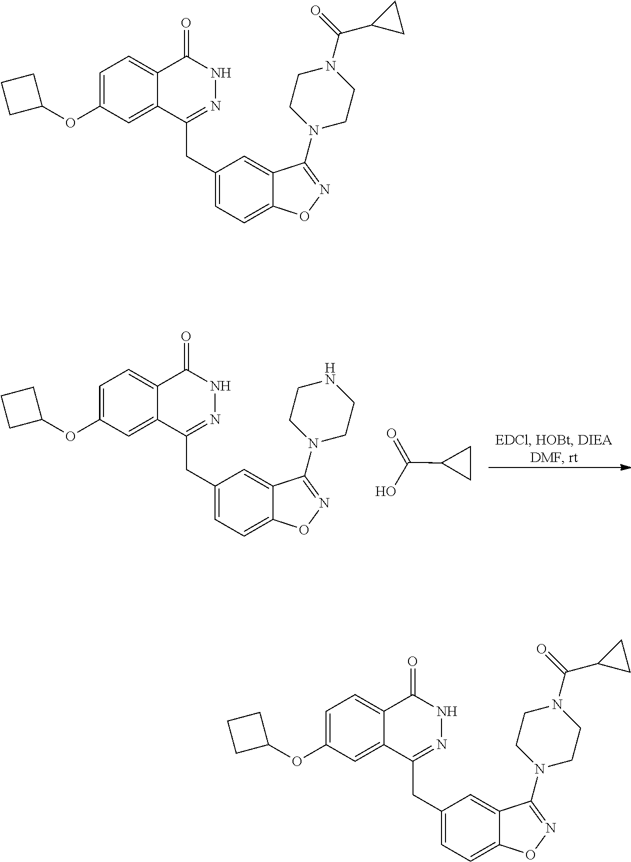

### Step 1

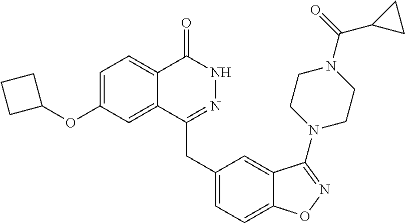 [SMILES: O=C(C1CC1)N1CCN(c2noc3ccc(Cc4n[nH]c(=O)c5ccc(OC6CCC6)cc45)cc23)CC1]

### 6-Cyclobutoxy-4-((3-(4-(cyclopropanecarbonyl)piperazin-1-yl)benzo[d]isoxazol-5-yl)methyl)phthalazin-1(2H)-one

To a solution of cyclopropanecarboxylic acid (24 mg, 0.28 mmol) and 6-cyclobutoxy-4-((3-(piperazin-1-yl)benzo[d]isoxazol-5-yl)methyl)phthalazin-1(2H)-one (120 mg, 0.26 mmol) in DMF (2 mL) was added EDCI (74 mg, 0.38 mmol), HOBt (52 mg, 0.38 mmol) and DIPEA (133 mg, 1.03 mmol). The reaction mixture was stirred at rt for 16 hours, then purified by prep-HPLC to afford 6-cyclobutoxy-4-((3-(4-(cyclopropanecarbonyl)piperazin-1-yl)benzo[d]isoxazol-5-yl)methyl)phthalazin-1(2H)-one (53 mg 41% yield) as a white solid. LCMS ESI 500 [M+H]⁺. ¹H NMR (400 MHz, DMSO-d₆) δ 12.44 (s, 1H), 8.20-8.10 (m, 2H), 7.50 (d, J=8.7 Hz, 1H), 7.44 (d, J=8.9 Hz, 1H), 7.30 (dd, J=8.8, 2.2 Hz, 1H), 7.19 (d, J=2.1 Hz, 1H), 4.85 (p, J=7.0 Hz, 1H), 4.40 (s, 2H), 3.89 (s, 2H), 3.69 (s, 2H), 3.50 (s, 4H), 2.41-2.32 (m, 2H), 2.12-1.95 (m, 3H), 1.78 (q, J=10.0 Hz, 1H), 1.64 (m, 1H), 0.81-0.67 (m, 4H).

### Synthesis of Example 211: 6-(4-(5-((7-cyclobutoxy-4-oxo-3,4-dihydrophthalazin-1-yl)methyl)benzo[d]isoxazol-3-yl)piperazin-1-yl)nicotinonitrile

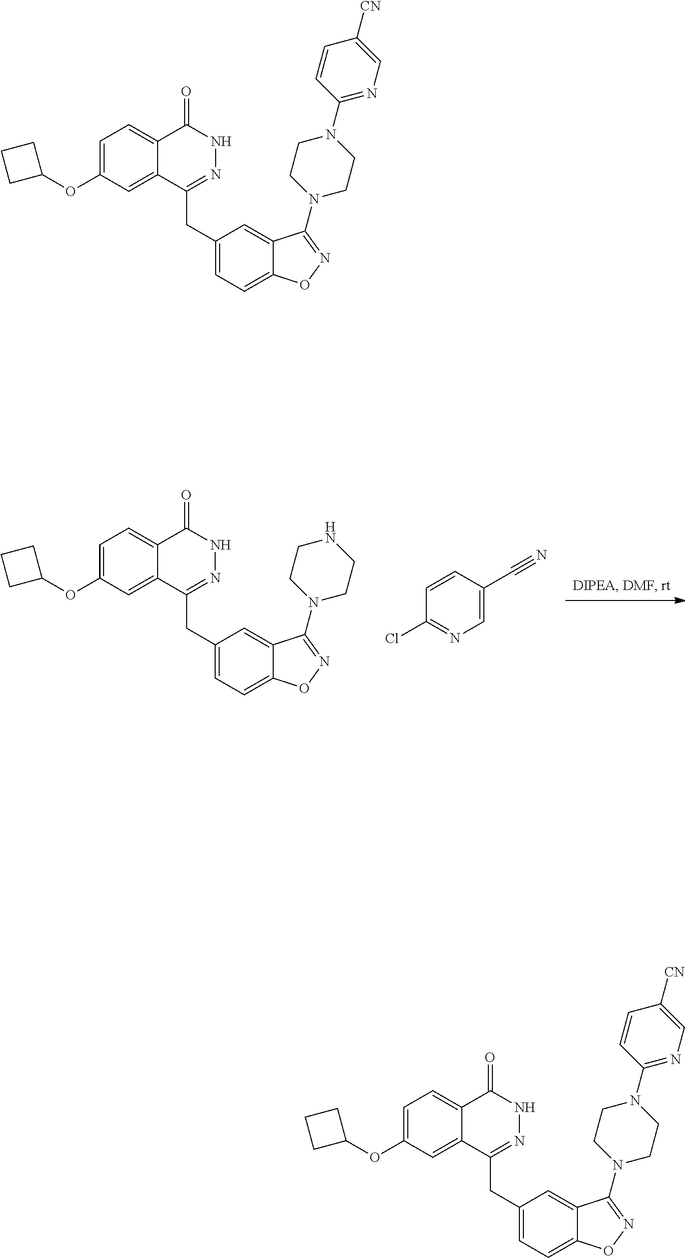 [SMILES: N#Cc1ccc(N2CCN(c3noc4ccc(Cc5n[nH]c(=O)c6ccc(OC7CCC7)cc56)cc34)CC2)nc1 ; O=c1[nH]nc(Cc2ccc3onc(N4CCNCC4)c3c2)c2cc(OC3CCC3)ccc12 ; N#Cc1ccc(Cl)nc1 ; N#Cc1ccc(N2CCN(c3noc4ccc(Cc5n[nH]c(=O)c6ccc(OC7CCC7)cc56)cc34)CC2)nc1]

### Step 1

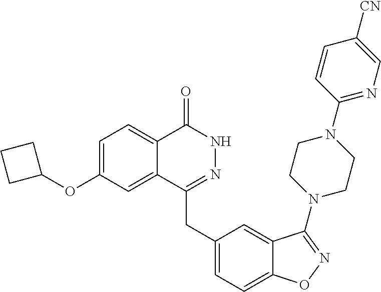 [SMILES: N#Cc1ccc(N2CCN(c3noc4ccc(Cc5n[nH]c(=O)c6ccc(OC7CCC7)cc56)cc34)CC2)nc1]

### 6-(4-(5-((7-Cyclobutoxy-4-oxo-3,4-dihydrophthalazin-1-yl)methyl)benzo [d]isoxazol-3-yl)piperazin-1-yl)nicotinonitrile

A solution of 6-cyclobutoxy-4-((3-(piperazin-1-yl)benzo[d]isoxazol-5-yl)methy 1)phthalazin-1(2H)-one (120 mg, 0.26 mmol), 6-chloronicotinonitrile (39 mg, 0.30 mmol) and DIPEA (133 mg, 1.00 mmol) in DMF (2 mL) was stirred at rt under Ar (g) for 12 hours, then purified by prep-HPLC to afford 6-(4-(5-((7-cyclobutoxy-4-oxo-3,4-dihydrophthalazin-1-yl)methyl)benzo[d]isoxazol-3-yl)piperazin-1-yl)nicotinonitrile (75 mg 55% yield) as a white solid. LCMS ESI 534 [M+H]⁺. ¹H NMR (400 MHz, DMSO-d₆) δ 12.44 (s, 1H), 8.54 (d, J=2.2 Hz, 1H), 8.15 (d, J=8.7 Hz, 2H), 7.92 (dd, J=9.1, 2.3 Hz, 1H), 7.57-7.39 (m, 2H), 7.30 (dd, J=8.8, 2.3 Hz, 1H), 7.19 (d, J=2.3 Hz, 1H), 7.03 (d, J=9.1 Hz, 1H), 4.85 (p, J=7.1 Hz, 1H), 4.42 (s, 2H), 3.97-3.84 (m, 4H), 3.68-3.52 (m, 4H), 2.37 (m, 2H), 2.04-1.92 (m, 2H), 1.69 (m, 2H).

### Synthesis of Example 212: 6-Cyclobutoxy-4-((3-(4-(5-(trifluoromethyl)pyrimidin-2-yl)piperazin-1-yl)benzo[d]isoxazol-6-yl)methyl)phthalazin-1(2H)-one

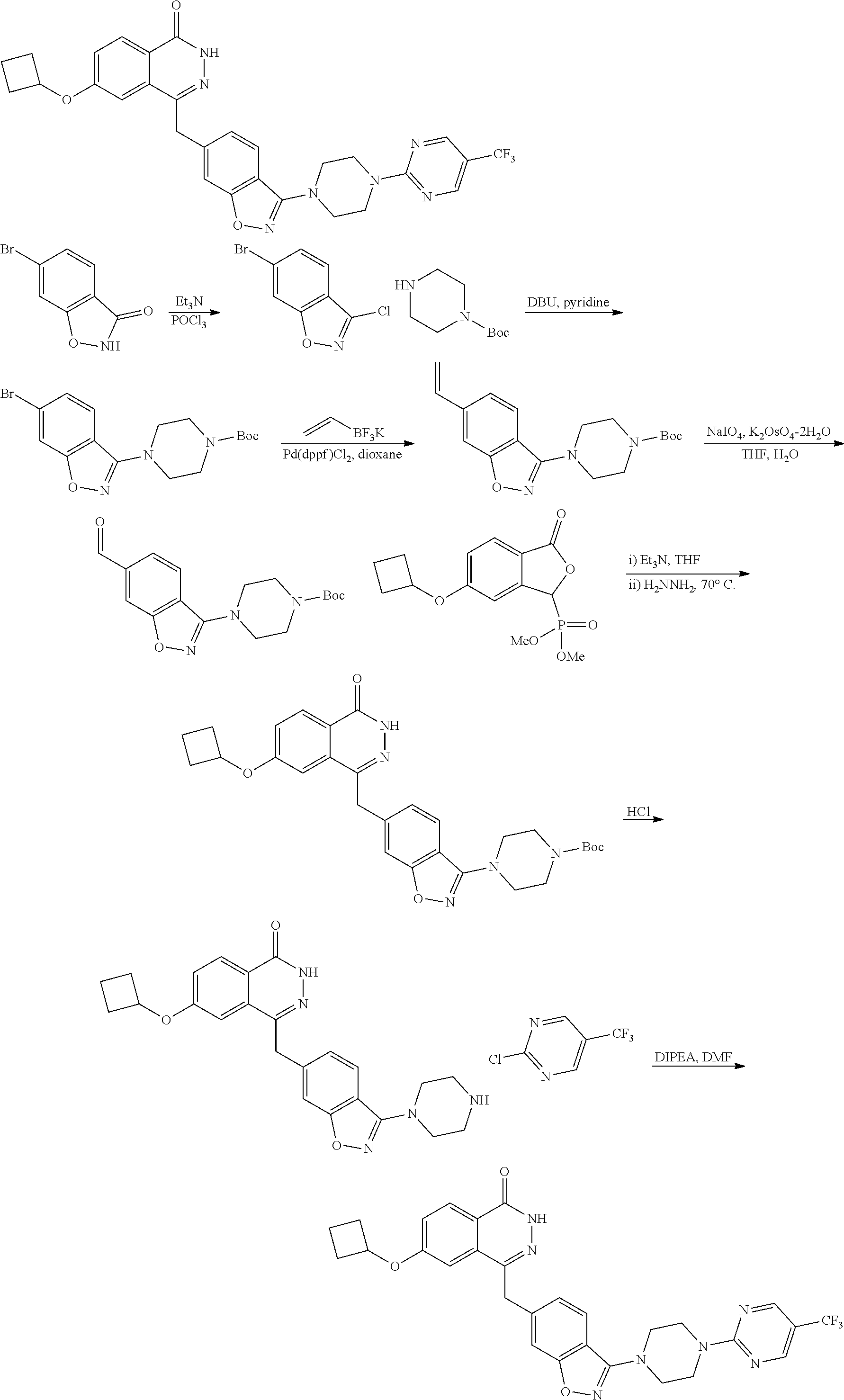

### Step 1

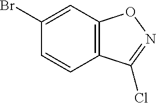 [SMILES: Clc1noc2cc(Br)ccc12]

### 6-Bromo-3-chlorobenzo[d]isoxazole

To a mixture of 6-bromobenzo[d]isoxazol-3(2H)-one (1.60 g, 7.48 mmol) in phosphoryl trichloride (20 mL) was added Et₃N (1.51 g, 14.95 mmol). The mixture was stirred at 130° C. for 12 hours. The reaction was carefully added to water (10 mL), then extracted with EtOAc (3×30 mL). The combined organic layer was washed with brine, dried over Na₂SO₄ and filtered. The solvent was removed in vacuo to give 6-bromo-3-chlorobenzo[d]isoxazole (1.16 g, 67% yield) as a white solid. LCMS ESI m/z: 232 [M+H]⁺.

### Step 2

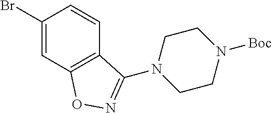 [SMILES: CC(C)(C)OC(=O)N1CCN(c2noc3cc(Br)ccc23)CC1]

### tert-Butyl 4-(6-bromobenzo[d]isoxazol-3-yl)piperazine-1-carboxylate

A mixture of 6-bromo-3-chlorobenzo[d]isoxazole (1.00 g, 4.30 mmol), tert-butyl piperazine-1-carboxylate (801 mg, 4.30 mmol) and 2,3,4,6,7,8,9,10-octahydropyrimido[1,2-a]azepine (786 mg, 5.16 mmol) in pyridine (20 mL) was stirred at 110° C. for 12 hours. The reaction was diluted with water (50 mL) and extracted with EtOAc (50 mL×3). The combined organic layer was washed with brine, dried over Na₂SO₄ and filtered. The solvent was removed in vacuo and the residue was purified by silica gel chromatography (EtOAc/petroleum ether=0% to 50%) to give tert-butyl 4-(6-bromobenzo[d]isoxazol-3-yl)piperazine-1-carboxylate (1.20 g, 73% yield) as a white solid. LCMS ESI m/z: 382 [M+H]⁺.

### Step 3

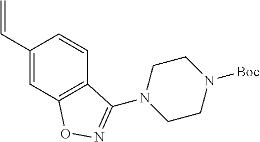 [SMILES: C=Cc1ccc2c(N3CCN(C(=O)OC(C)(C)C)CC3)noc2c1]

### tert-butyl 4-(6-vinylbenzo[d]isoxazol-3-yl)piperazine-1-carboxylate

A mixture of tert-butyl 4-(6-bromobenzo[d]isoxazol-3-yl)piperazine-1-carboxy late (1.20 g, 3.14 mmol), potassium vinyltrifluoroborate (505 mg, 3.77 mmol), Pd(dppf)Cl₂ (230 mg, 0.31 mmol) and K₂CO₃ (1.08 g, 7.85 mmol) in dioxane (10 mL) was stirred at 80° C. for 5 hours. The mixture was cooled to rt, diluted with H₂O (20 mL) and extracted with EtOAc (3×20 mL). The combined organic layer was washed with brine, dried over Na₂SO₄ and filtered. The solvent was removed in vacuo and the residue was purified by silica gel chromatography (EtOAc/petroleum ether=0% to 30%) to give tert-butyl 4-(6-vinylbenzo[d]isoxazol-3-yl)piperazine-1-carboxylate (710 mg, 69% yield) as a white solid. LCMS ESI m/z: 330 [M+H]⁺.

### Step 4

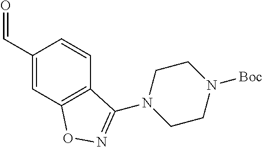 [SMILES: CC(C)(C)OC(=O)N1CCN(c2noc3cc(C=O)ccc23)CC1]

### tert-butyl 4-(6-formylbenzo[d]isoxazol-3-yl)piperazine-1-carboxylate

To a mixture of tert-butyl 4-(6-formylbenzo[d]isoxazol-3-yl)piperazine-1-carboxylate (700 mg, 1.99 mmol) in THF (9 mL) and H₂O (6 mL) was added K₂OsO₄-2H₂O (37 mg, 0.10 mmol). The mixture was stirred at rt for 5 min, added NaIO₄ (1.28 g, 5.96 mmol) and stirred at rt for 4 hours. Then THF was removed under reduced pressure, diluted with H₂O (20 mL) and extracted with EtOAc (3×20 mL). The combined organic layer was washed with brine, dried over Na2SO₄ and filtered. The solvent was removed in vacuo to give tert-butyl 4-(6-vinylbenzo[d]isoxazol-3-yl)piperazine-1-carboxylate (710 mg, 69% yield) as a white solid. This was used without further purification. LCMS ESI m/z: 332 [M+H]⁺.

### Step 5

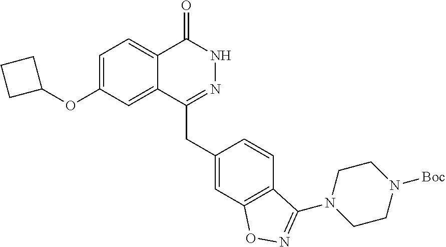 [SMILES: CC(C)(C)OC(=O)N1CCN(c2noc3cc(Cc4n[nH]c(=O)c5ccc(OC6CCC6)cc45)ccc23)CC1]

### tert-butyl 4-(6-((7-cyclobutoxy-4-oxo-3,4-dihydrophthalazin-1-yl)methyl)benzo[d]isoxazol-3-yl)piperazine-1-carboxylate

A solution of tert-butyl 4-(6-formylbenzo[d]isoxazol-3-yl)piperazine-1-carboxylate (450 mg, 1.36 mmol), dimethyl (6-cyclobutoxy-3-oxo-1,3-dihydroisobenzofuran-1-yl)phosphonate (636 mg, 2.04 mmol) and Et3N (412 mg, 4.07 mmol) in THF (5 mL) was stirred at 25° C. under Ar (g) for 15 hours. Hydrazine hydrate (102 mg, 2.04 mmol) was added and the reaction was stirred at 70° C. for 1 hour. The reaction was cooled to rt, and the solvent was removed under vacuum. The formed solids were washed with water (10 mL) and petroleum ether/EtOAc (1:1, 10 mL) to give tert-butyl 4-(6-((7-cyclobutoxy-4-oxo-3,4-dihydrophthalazin-1-yl)methyl)benzo[d]isoxazol-3-yl)piperazine-1-carboxylate (430 mg, 60% yield) as a white solid. LCMS ESI m/z: 532 [M+H]+.

### Step 6

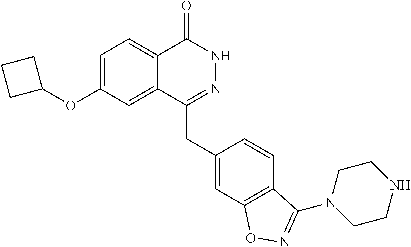 [SMILES: O=c1[nH]nc(Cc2ccc3c(N4CCNCC4)noc3c2)c2cc(OC3CCC3)ccc12]

### 6-cyclobutoxy-4-((3-(piperazin-1-yl)benzo[d]isoxazol-6-yl)methyl)phthalazin-1(2H)-one

A solution of tert-butyl 4-(6-((7-cyclobutoxy-4-oxo-3,4-dihydrophthalazin-1-yl)methyl)benzo[d]isoxazol-3-yl)piperazine-1-carboxylate (90 mg, 0.17 mmol) in HCl/dioxane (4M, 5 mL) was stirred at rt for 1 hour. The solvent was removed in vacuo to give 6-cyclobutoxy-4-((3-(piperazin-1-yl)benzo[d]isoxazol-6-yl)methyl)phthalazin-1(2H)-one (70 mg, 94% yield) as a white solid. This was used without further purification. LCMS ESI m/z: 532 [M+H]⁺.

### Step 7

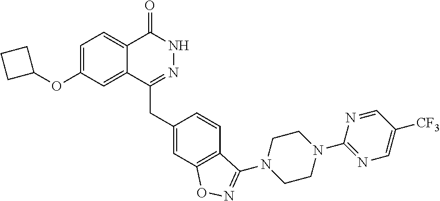 [SMILES: O=c1[nH]nc(Cc2ccc3c(N4CCN(c5ncc(C(F)(F)F)cn5)CC4)noc3c2)c2cc(OC3CCC3)ccc12]

### 6-cyclobutoxy-4-((3-(4-(5-(trifluoromethyl)pyrimidin-2-yl)piperazin-1-yl)benzo[d]isoxazol-6-yl)methyl)phthalazin-1(2H)-one

To a solution of 6-cyclobutoxy-4-((3-(piperazin-1-yl)benzo[d]isoxazol-6-yl)methyl)phthalazin-1(2H)-one (60 mg, 0.14 mmol) and 2-chloro-5-(trifluoromethyl)pyrimidine (25 mg, 0.14 mmol) in DMF (1 mL) was added DIPEA (72 mg, 0.56 mmol). The reaction mixture was stirred at 60° C. for 2 hours. The mixture was purified by prep-HPLC to give 6-cyclobutoxy-4-((3-(4-(5-(trifluoromethyl)pyrimidin-2-yl)piperazin-1-yl)benzo[d]isoxazol-6-yl)methyl) phthalazin-1(2H)-one (60 mg, 75% yield) as a white solid. LCMS ESI m/z: 532 [M+H]⁺. ¹H-NMR (400 MHz, DMSO-d₆) δ 12.46 (s, 1H), 8.75 (s, 2H), 8.15 (d, J=8.8 Hz, 1H), 7.98-7.94 (m, 1H), 7.58 (s, 1H), 7.32-7.28 (m, 1H), 7.27-7.24 (m, 1H), 7.15-7.12 (m, 1H), 4.87-4.79 (m, 1H), 4.43 (s, 2H), 4.06-3.99 (m, 4H), 3.59-3.54 (m, 4H), 2.41-2.35 (m, 2H), 2.03-1.93 (m, 2H), 1.83-1.73 (m, 1H), 1.69-1.59 (m, 1H).

### Synthesis of Example 213: 6-cyclobutoxy-4-((3-(4-(cyclopropanecarbonyl)piperazin-1-yl)benzo[d]isoxazol-6-yl)methyl)phthalazin-1(2H)-one

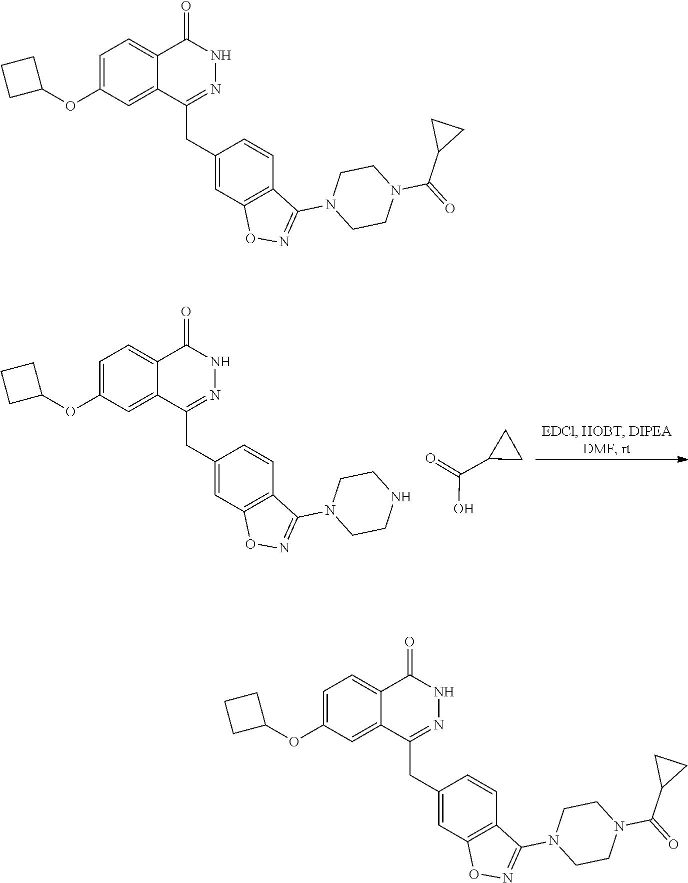 [SMILES: O=C(C1CC1)N1CCN(c2noc3cc(Cc4n[nH]c(=O)c5ccc(OC6CCC6)cc45)ccc23)CC1 ; O=c1[nH]nc(Cc2ccc3c(N4CCNCC4)noc3c2)c2cc(OC3CCC3)ccc12 ; O=C(O)C1CC1 ; O=C(C1CC1)N1CCN(c2noc3cc(Cc4n[nH]c(=O)c5ccc(OC6CCC6)cc45)ccc23)CC1]

### Step 1

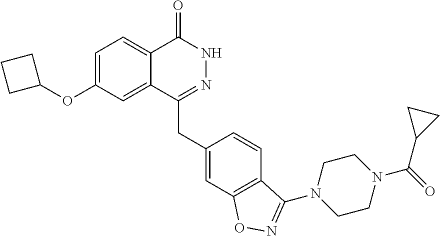 [SMILES: O=C(C1CC1)N1CCN(c2noc3cc(Cc4n[nH]c(=O)c5ccc(OC6CCC6)cc45)ccc23)CC1]

### 6-cyclobutoxy-4-((3-(4-(cyclopropanecarbonyl)piperazin-1-yl)benzo[d]isoxazol-6-yl)methyl)phthalazin-1(2H)-one

To a solution of 6-cyclobutoxy-4-((3-piperazin-1-yl)benzo[d]isoxazol-6-yl)methyl)phthalazin-1(2H)-one (110 mg, 0.34 mmol) and cyclopropanecarboxylic acid (20 mg, 0.23 mmol) in DMF (2 mL) was added EDCI (68 mg, 0.35 mmol). HOBt (48 mg, 0.35 mmol) and DIPEA (122 mg, 0.94 mmol). The reaction mixture was stirred at rt for 12 hours, then purified by prep-HPLC to afford 6-cyclobutoxy-4-((3-(4-(cyclopropanecarbonyl)piperazin-1-yl)benzo[d]isoxazol-6-yl)methyl)phthalazin-1(2H)-one (80 mg 68% yield) as a white solid. LCMS ESI 500.3 [M+H]⁺. ¹H NMR (400 MHz, DMSO-d₆) δ 12.47 (s, 1H), 8.15 (d, J=8.8 Hz, 1H), 7.93 (d, J=8.3 Hz, 1H), 7.58 (s, 1H), 7.30 (dd, J=8.8, 2.3 Hz, 1H), 7.25 (d, J=8.3 Hz, 1H), 7.14 (d, J=2.3 Hz, 1H), 4.84 (dd, J=14.1, 7.1 Hz, 1H), 4.43 (s, 2H), 3.86 (s, 2H), 3.65 (s, 2H), 3.46 (d, J=22.6 Hz, 4H), 2.36 (dd, J=10.1, 7.3 Hz, 2H), 2.04-1.95 (m, 3H), 1.83-1.73 (m, 1H), 1.64 (dd, J=18.8, 8.5 Hz, 1H), 0.77-0.72 (m, 4H).

### Synthesis of Example 214: 6-(4-(6-((7-Cyclobutoxy-4-oxo-3,4-dihydrophthalazin-1-yl)methyl)benzo[d]isoxazol-3-yl)piperazin-1-yl)nicotinonitrile

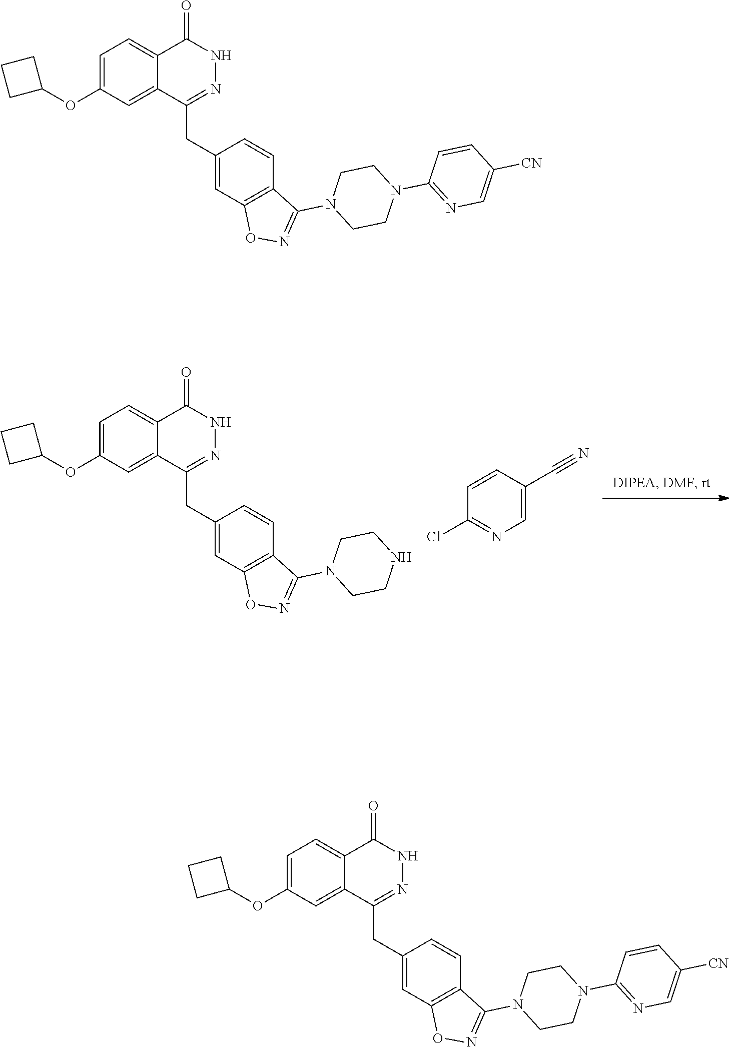 [SMILES: N#Cc1ccc(N2CCN(c3noc4cc(Cc5n[nH]c(=O)c6ccc(OC7CCC7)cc56)ccc34)CC2)nc1 ; O=c1[nH]nc(Cc2ccc3c(N4CCNCC4)noc3c2)c2cc(OC3CCC3)ccc12 ; N#Cc1ccc(Cl)nc1 ; N#Cc1ccc(N2CCN(c3noc4cc(Cc5n[nH]c(=O)c6ccc(OC7CCC7)cc56)ccc34)CC2)nc1]

### Step 1

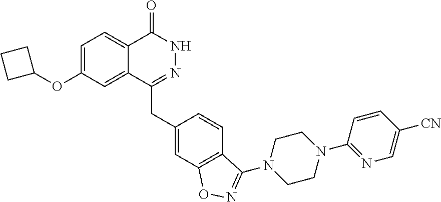 [SMILES: N#Cc1ccc(N2CCN(c3noc4cc(Cc5n[nH]c(=O)c6ccc(OC7CCC7)cc56)ccc34)CC2)nc1]

### 6-(4-(6-((7-Cyclobutoxy-4-oxo-3,4-dihydrophthalazin-1-yl)methyl)benzo[d]isoxazol-3-yl)piperazin-1-yl)nicotinonitrile

A solution of 6-cyclobutoxy-4-((3-(piperazin-1-yl)benzo[d]isoxazol-6-yl)methyl)phthalazin-1(2H)-one (120 mg, 0.30 mmol), 6-chloronicotinonitrile (71 mg, 0.50 mmol) and DIPEA (132 mg, 1.20 mmol) in DMF (2 mL) was stirred at rt under Ar (g) for 12 hours, then purified by prep-HPLC to afford 6-(4-(6-((7-cyclobutoxy-4-oxo-3,4-dihydrophthalazin-1-yl)methyl)benzo[d]isoxazol-3-yl)piperazin-1-yl)nicotinonitrile diazepane (60 mg 41% yield) as a white solid. LCMS ESI 534.2 [M+H]⁺. ¹H NMR (400 MHz, DMSO-d₆) δ 12.47 (s, 1H), 8.53 (d, J=2.3 Hz, 1H), 8.15 (d, J=8.8 Hz, 1H), 7.98-7.87 (m, 2H), 7.59 (s, 1H), 7.31-7.24 (m, 2H), 7.14 (d, J=2.3 Hz, 1H), 7.01 (d, J=9.1 Hz, 1H), 4.91-4.75 (m, 1H), 4.43 (s, 2H), 3.96-3.82 (m, 4H), 3.61-3.49 (m, 4H), 2.40-2.32 (m, 2H), 2.04-1.91 (m, 2H), 1.70 (m, 2H).

### Results

TABLE 2
Example Compounds

| Example | Structure |
|---|---|
| 1 | 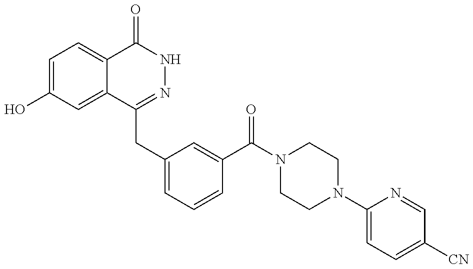 [SMILES: N#Cc1ccc(N2CCN(C(=O)c3cccc(Cc4n[nH]c(=O)c5ccc(O)cc45)c3)CC2)nc1] |
| 2 | 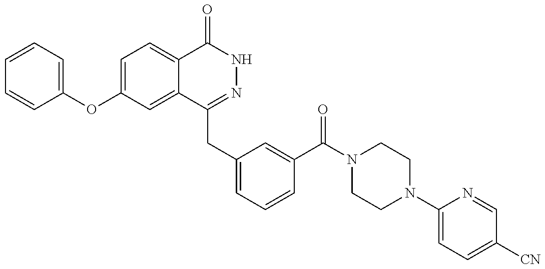 [SMILES: N#Cc1ccc(N2CCN(C(=O)c3cccc(Cc4n[nH]c(=O)c5ccc(Oc6ccccc6)cc45)c3)CC2)nc1] |
| 3 | 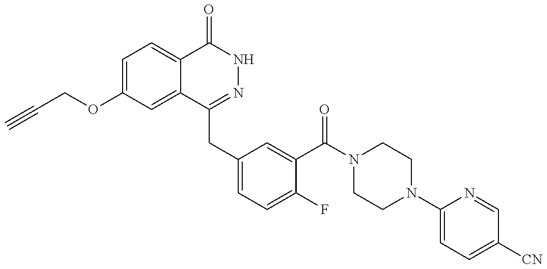 [SMILES: C#CCOc1ccc2c(=O)[nH]nc(Cc3ccc(F)c(C(=O)N4CCN(c5ccc(C#N)cn5)CC4)c3)c2c1] |
| 4 | 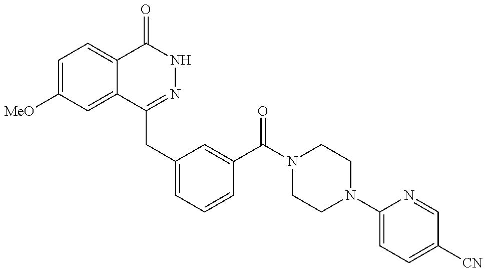 [SMILES: COc1ccc2c(=O)[nH]nc(Cc3cccc(C(=O)N4CCN(c5ccc(C#N)cn5)CC4)c3)c2c1] |
| 5 | 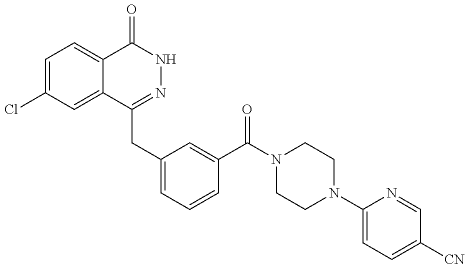 [SMILES: N#Cc1ccc(N2CCN(C(=O)c3cccc(Cc4n[nH]c(=O)c5ccc(Cl)cc45)c3)CC2)nc1] |
| 6 | 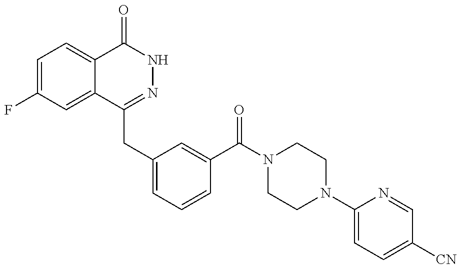 [SMILES: N#Cc1ccc(N2CCN(C(=O)c3cccc(Cc4n[nH]c(=O)c5ccc(F)cc45)c3)CC2)nc1] |
| 7 | 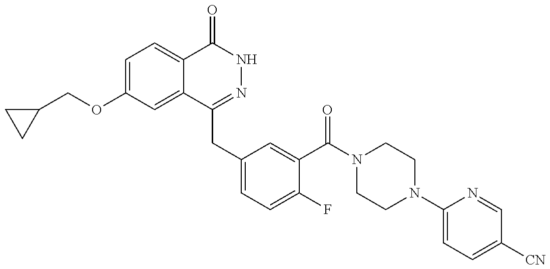 [SMILES: N#Cc1ccc(N2CCN(C(=O)c3cc(Cc4n[nH]c(=O)c5ccc(OCC6CC6)cc45)ccc3F)CC2)nc1] |
| 8 | 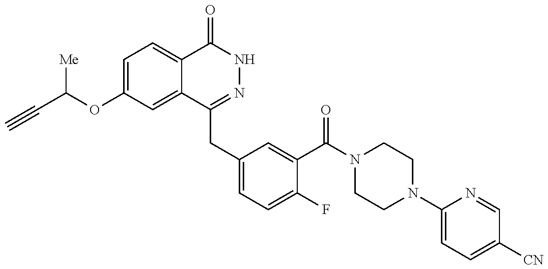 [SMILES: C#CC(C)Oc1ccc2c(=O)[nH]nc(Cc3ccc(F)c(C(=O)N4CCN(c5ccc(C#N)cn5)CC4)c3)c2c1] |
| 9 | 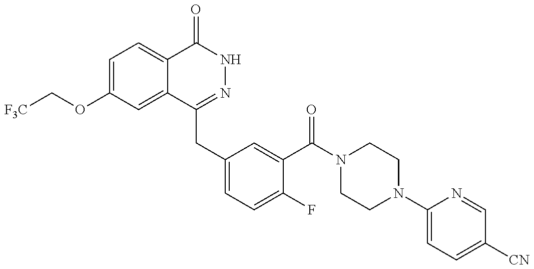 [SMILES: N#Cc1ccc(N2CCN(C(=O)c3cc(Cc4n[nH]c(=O)c5ccc(OCC(F)(F)F)cc45)ccc3F)CC2)nc1] |
| 10 | 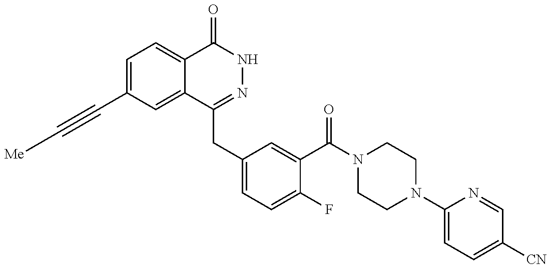 [SMILES: CC#Cc1ccc2c(=O)[nH]nc(Cc3ccc(F)c(C(=O)N4CCN(c5ccc(C#N)cn5)CC4)c3)c2c1] |
| 11 | 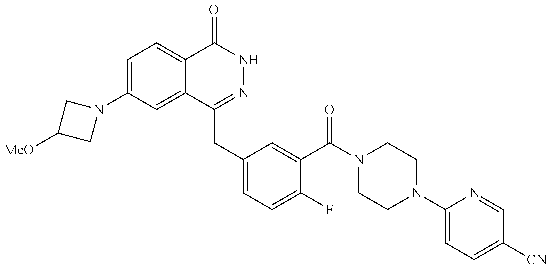 [SMILES: COC1CN(c2ccc3c(=O)[nH]nc(Cc4ccc(F)c(C(=O)N5CCN(c6ccc(C#N)cn6)CC5)c4)c3c2)C1] |
| 12 | 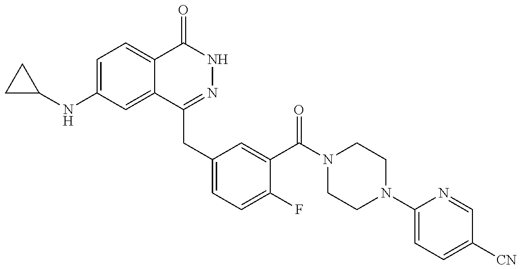 [SMILES: N#Cc1ccc(N2CCN(C(=O)c3cc(Cc4n[nH]c(=O)c5ccc(NC6CC6)cc45)ccc3F)CC2)nc1] |
| 13 | 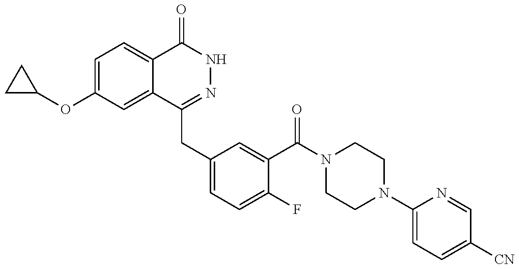 [SMILES: N#Cc1ccc(N2CCN(C(=O)c3cc(Cc4n[nH]c(=O)c5ccc(OC6CC6)cc45)ccc3F)CC2)nc1] |
| 14 | 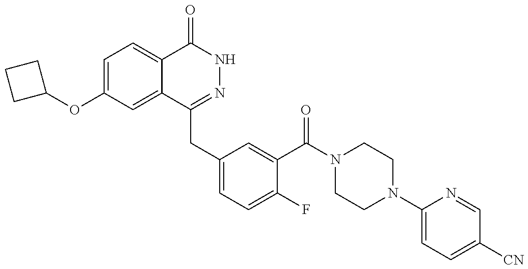 [SMILES: N#Cc1ccc(N2CCN(C(=O)c3cc(Cc4n[nH]c(=O)c5ccc(OC6CCC6)cc45)ccc3F)CC2)nc1] |
| 15 | 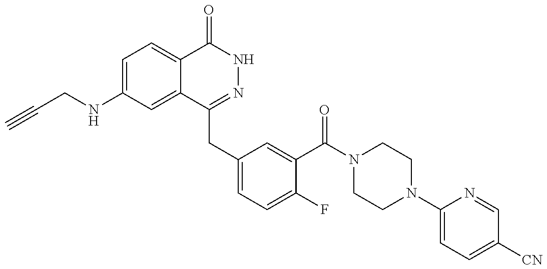 [SMILES: C#CCNc1ccc2c(=O)[nH]nc(Cc3ccc(F)c(C(=O)N4CCN(c5ccc(C#N)cn5)CC4)c3)c2c1] |
| 16 | 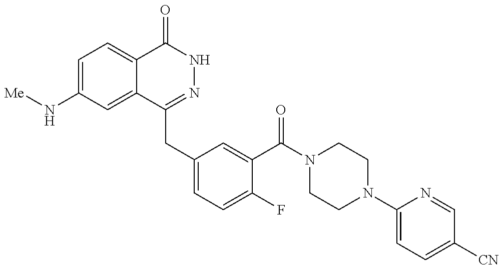 [SMILES: CNc1ccc2c(=O)[nH]nc(Cc3ccc(F)c(C(=O)N4CCN(c5ccc(C#N)cn5)CC4)c3)c2c1] |
| 17 | 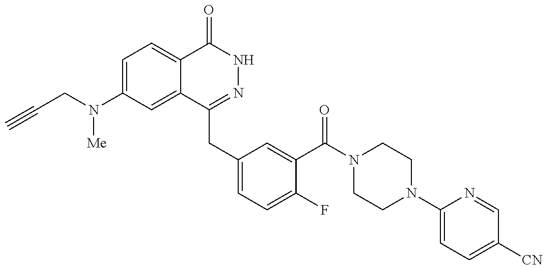 [SMILES: C#CCN(C)c1ccc2c(=O)[nH]nc(Cc3ccc(F)c(C(=O)N4CCN(c5ccc(C#N)cn5)CC4)c3)c2c1] |
| 18 | 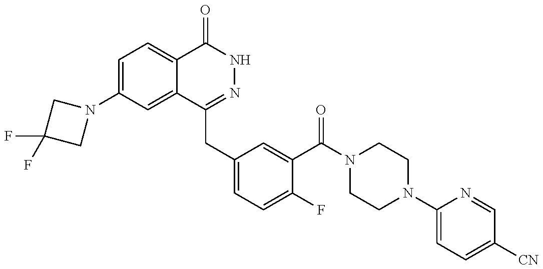 [SMILES: N#Cc1ccc(N2CCN(C(=O)c3cc(Cc4n[nH]c(=O)c5ccc(N6CC(F)(F)C6)cc45)ccc3F)CC2)nc1] |
| 19 | 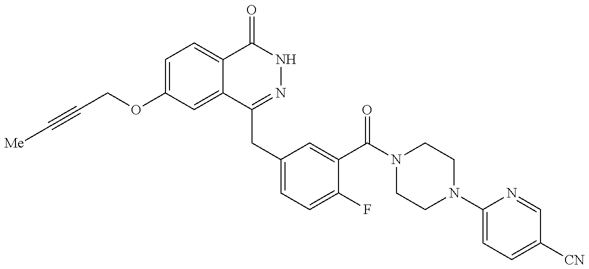 [SMILES: CC#CCOc1ccc2c(=O)[nH]nc(Cc3ccc(F)c(C(=O)N4CCN(c5ccc(C#N)cn5)CC4)c3)c2c1] |
| 20 | 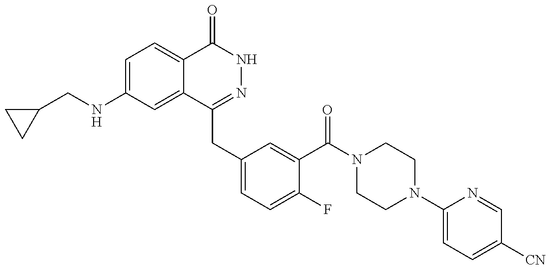 [SMILES: N#Cc1ccc(N2CCN(C(=O)c3cc(Cc4n[nH]c(=O)c5ccc(NCC6CC6)cc45)ccc3F)CC2)nc1] |
| 21 | 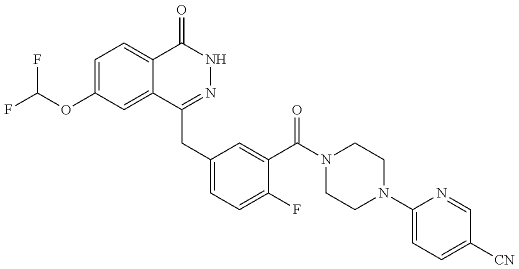 [SMILES: N#Cc1ccc(N2CCN(C(=O)c3cc(Cc4n[nH]c(=O)c5ccc(OC(F)F)cc45)ccc3F)CC2)nc1] |
| 22 | 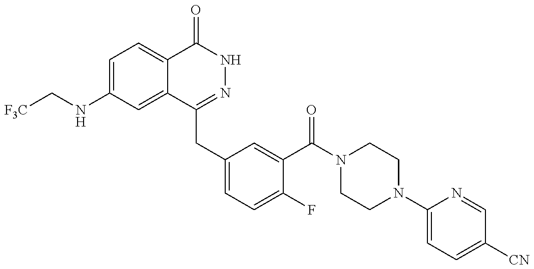 [SMILES: N#Cc1ccc(N2CCN(C(=O)c3cc(Cc4n[nH]c(=O)c5ccc(NCC(F)(F)F)cc45)ccc3F)CC2)nc1] |
| 23 | 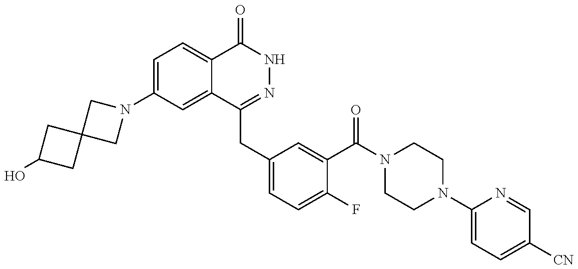 [SMILES: N#Cc1ccc(N2CCN(C(=O)c3cc(Cc4n[nH]c(=O)c5ccc(N6CC7(CC(O)C7)C6)cc45)ccc3F)CC2)nc1] |
| 24 | 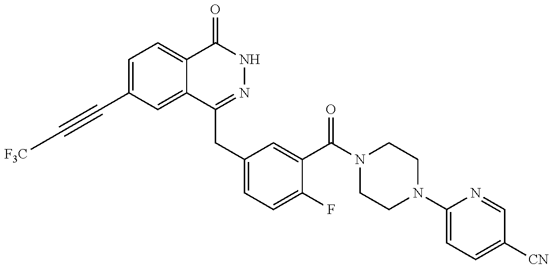 [SMILES: N#Cc1ccc(N2CCN(C(=O)c3cc(Cc4n[nH]c(=O)c5ccc(C#CC(F)(F)F)cc45)ccc3F)CC2)nc1] |
| 25 | 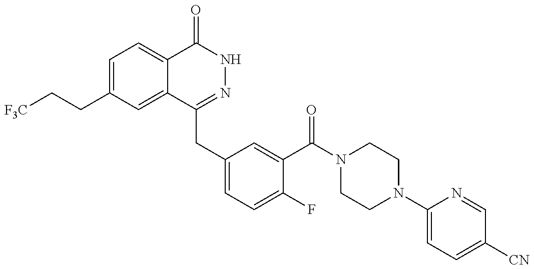 [SMILES: N#Cc1ccc(N2CCN(C(=O)c3cc(Cc4n[nH]c(=O)c5ccc(CCC(F)(F)F)cc45)ccc3F)CC2)nc1] |
| 26 | 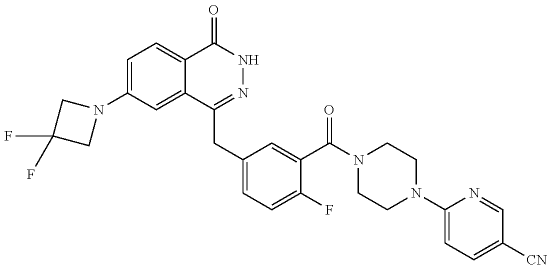 [SMILES: N#Cc1ccc(N2CCN(C(=O)c3cc(Cc4n[nH]c(=O)c5ccc(N6CC(F)(F)C6)cc45)ccc3F)CC2)nc1] |
| 27 | 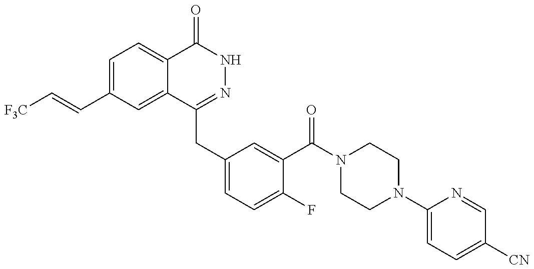 [SMILES: N#Cc1ccc(N2CCN(C(=O)c3cc(Cc4n[nH]c(=O)c5ccc(/C=C/C(F)(F)F)cc45)ccc3F)CC2)nc1] |
| 28 | 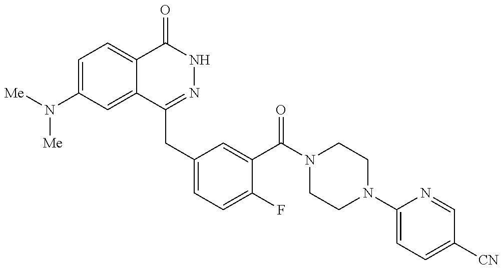 [SMILES: CN(C)c1ccc2c(=O)[nH]nc(Cc3ccc(F)c(C(=O)N4CCN(c5ccc(C#N)cn5)CC4)c3)c2c1] |
| 29 | 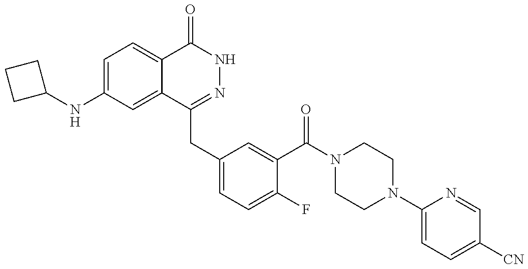 [SMILES: N#Cc1ccc(N2CCN(C(=O)c3cc(Cc4n[nH]c(=O)c5ccc(NC6CCC6)cc45)ccc3F)CC2)nc1] |
| 30 | 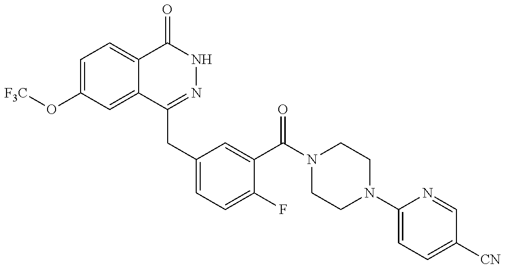 [SMILES: N#Cc1ccc(N2CCN(C(=O)c3cc(Cc4n[nH]c(=O)c5ccc(OC(F)(F)F)cc45)ccc3F)CC2)nc1] |
| 31 | 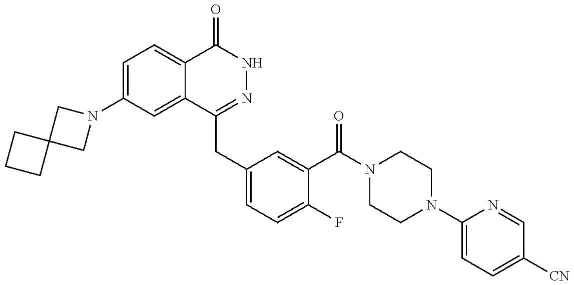 [SMILES: N#Cc1ccc(N2CCN(C(=O)c3cc(Cc4n[nH]c(=O)c5ccc(N6CC7(CCC7)C6)cc45)ccc3F)CC2)nc1] |
| 32 | 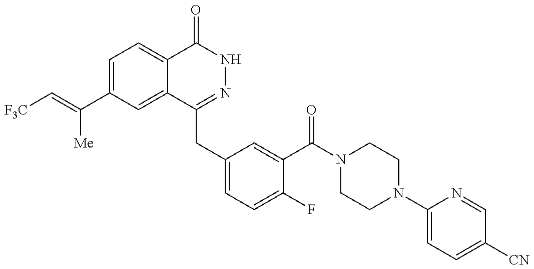 [SMILES: C/C(=C\C(F)(F)F)c1ccc2c(=O)[nH]nc(Cc3ccc(F)c(C(=O)N4CCN(c5ccc(C#N)cn5)CC4)c3)c2c1] |
| 33 | 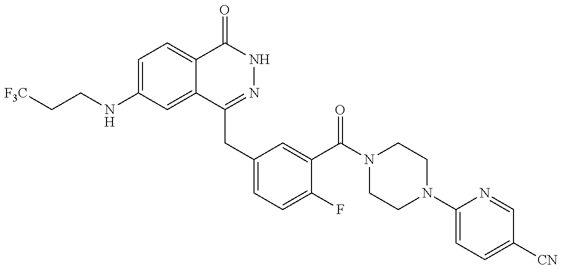 [SMILES: N#Cc1ccc(N2CCN(C(=O)c3cc(Cc4n[nH]c(=O)c5ccc(NCCC(F)(F)F)cc45)ccc3F)CC2)nc1] |
| 34 | 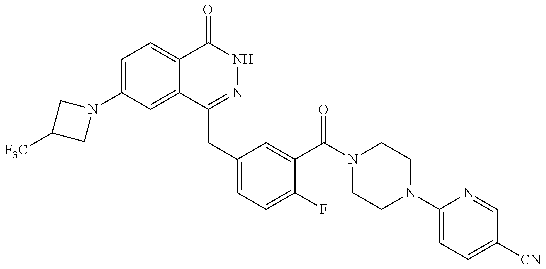 [SMILES: N#Cc1ccc(N2CCN(C(=O)c3cc(Cc4n[nH]c(=O)c5ccc(N6CC(C(F)(F)F)C6)cc45)ccc3F)CC2)nc1] |
| 35 | 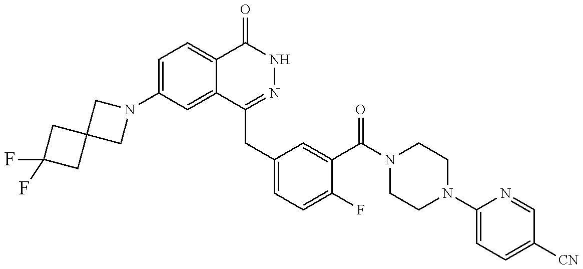 [SMILES: N#Cc1ccc(N2CCN(C(=O)c3cc(Cc4n[nH]c(=O)c5ccc(N6CC7(C6)CC(F)(F)C7)cc45)ccc3F)CC2)nc1] |
| 36 | 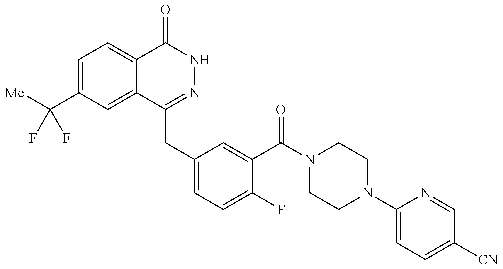 [SMILES: CC(F)(F)c1ccc2c(=O)[nH]nc(Cc3ccc(F)c(C(=O)N4CCN(c5ccc(C#N)cn5)CC4)c3)c2c1] |
| 37 | 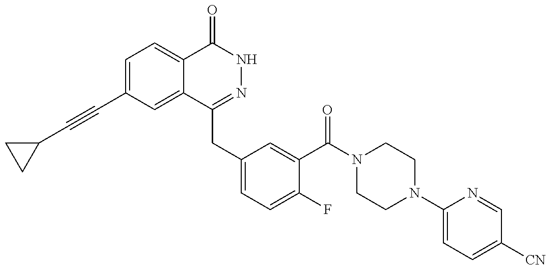 [SMILES: N#Cc1ccc(N2CCN(C(=O)c3cc(Cc4n[nH]c(=O)c5ccc(C#CC6CC6)cc45)ccc3F)CC2)nc1] |
| 38 | 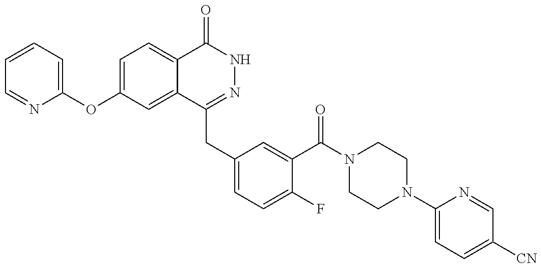 [SMILES: N#Cc1ccc(N2CCN(C(=O)c3cc(Cc4n[nH]c(=O)c5ccc(Oc6ccccn6)cc45)ccc3F)CC2)nc1] |
| 39 | 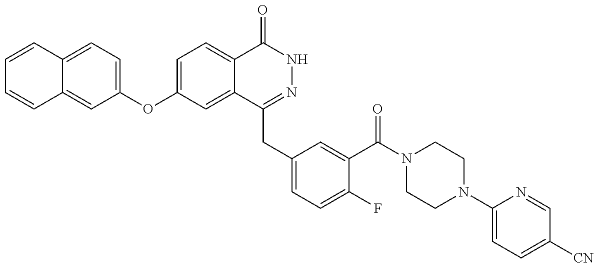 [SMILES: N#Cc1ccc(N2CCN(C(=O)c3cc(Cc4n[nH]c(=O)c5ccc(Oc6ccc7ccccc7c6)cc45)ccc3F)CC2)nc1] |
| 40 | 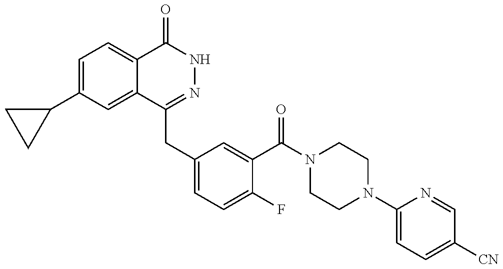 [SMILES: N#Cc1ccc(N2CCN(C(=O)c3cc(Cc4n[nH]c(=O)c5ccc(C6CC6)cc45)ccc3F)CC2)nc1] |
| 41 | 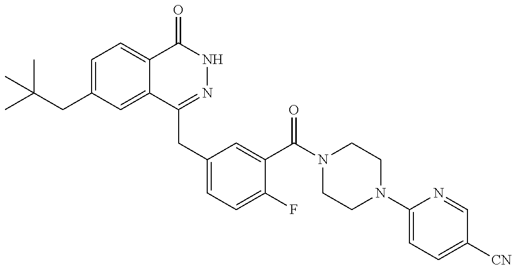 [SMILES: CC(C)(C)Cc1ccc2c(=O)[nH]nc(Cc3ccc(F)c(C(=O)N4CCN(c5ccc(C#N)cn5)CC4)c3)c2c1] |
| 42 | 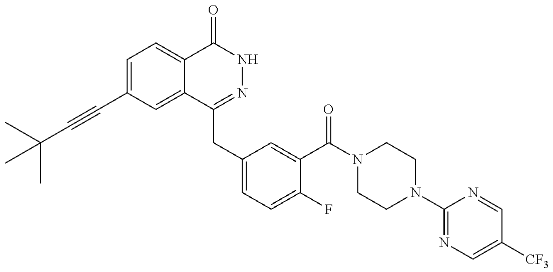 [SMILES: CC(C)(C)C#Cc1ccc2c(=O)[nH]nc(Cc3ccc(F)c(C(=O)N4CCN(c5ncc(C(F)(F)F)cn5)CC4)c3)c2c1] |
| 43 | 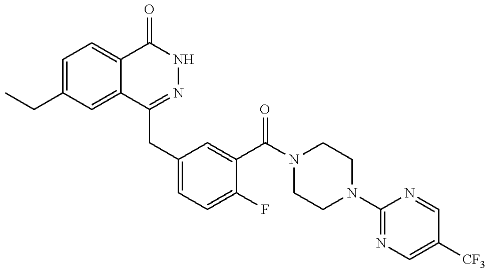 [SMILES: CCc1ccc2c(=O)[nH]nc(Cc3ccc(F)c(C(=O)N4CCN(c5ncc(C(F)(F)F)cn5)CC4)c3)c2c1] |
| 44 | 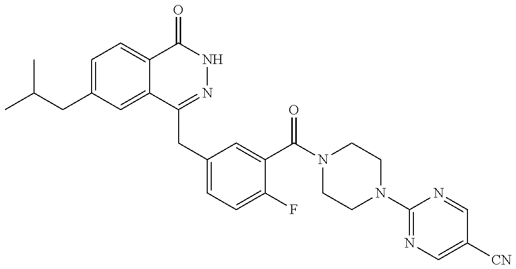 [SMILES: CC(C)Cc1ccc2c(=O)[nH]nc(Cc3ccc(F)c(C(=O)N4CCN(c5ncc(C#N)cn5)CC4)c3)c2c1] |
| 45 | 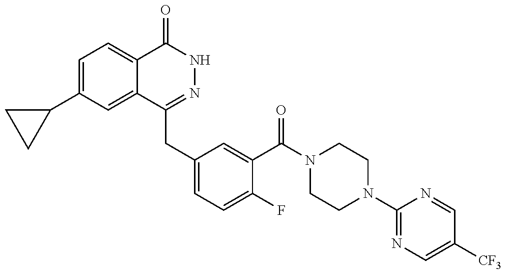 [SMILES: O=C(c1cc(Cc2n[nH]c(=O)c3ccc(C4CC4)cc23)ccc1F)N1CCN(c2ncc(C(F)(F)F)cn2)CC1] |
| 46 | 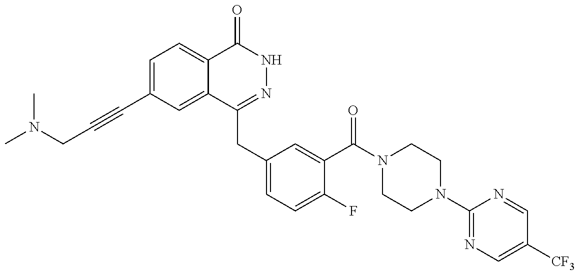 [SMILES: CN(C)CC#Cc1ccc2c(=O)[nH]nc(Cc3ccc(F)c(C(=O)N4CCN(c5ncc(C(F)(F)F)cn5)CC4)c3)c2c1] |
| 47 | 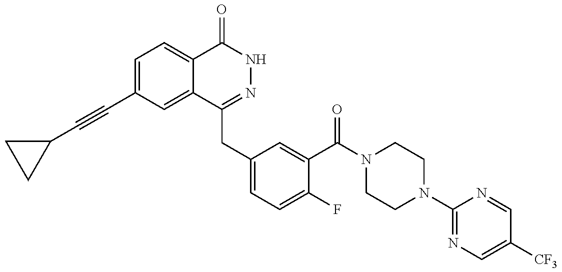 [SMILES: O=C(c1cc(Cc2n[nH]c(=O)c3ccc(C#CC4CC4)cc23)ccc1F)N1CCN(c2ncc(C(F)(F)F)cn2)CC1] |
| 48 | 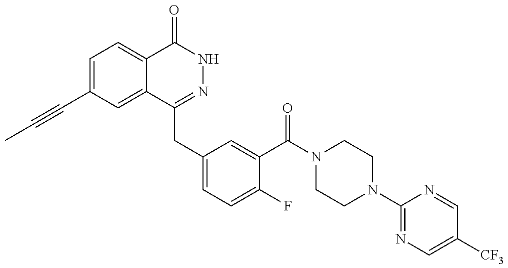 [SMILES: CC#Cc1ccc2c(=O)[nH]nc(Cc3ccc(F)c(C(=O)N4CCN(c5ncc(C(F)(F)F)cn5)CC4)c3)c2c1] |
| 49 | 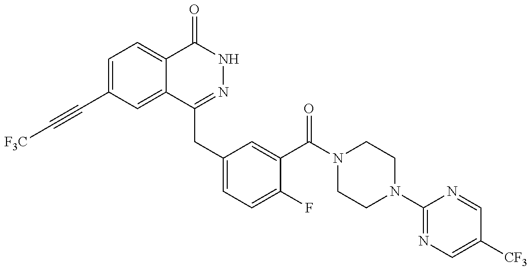 [SMILES: O=C(c1cc(Cc2n[nH]c(=O)c3ccc(C#CC(F)(F)F)cc23)ccc1F)N1CCN(c2ncc(C(F)(F)F)cn2)CC1] |
| 51 | 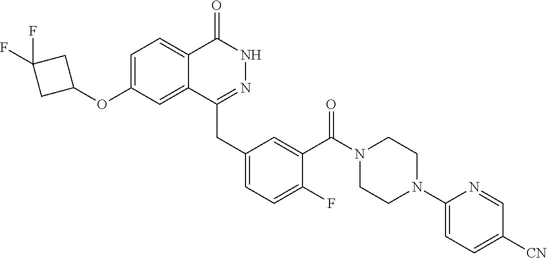 [SMILES: N#Cc1ccc(N2CCN(C(=O)c3cc(Cc4n[nH]c(=O)c5ccc(OC6CC(F)(F)C6)cc45)ccc3F)CC2)nc1] |
| 52 | 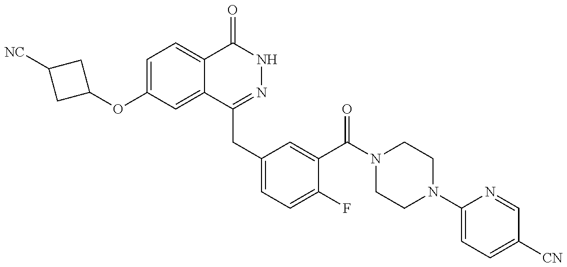 [SMILES: N#Cc1ccc(N2CCN(C(=O)c3cc(Cc4n[nH]c(=O)c5ccc(OC6CC(C#N)C6)cc45)ccc3F)CC2)nc1] |
| 53 | 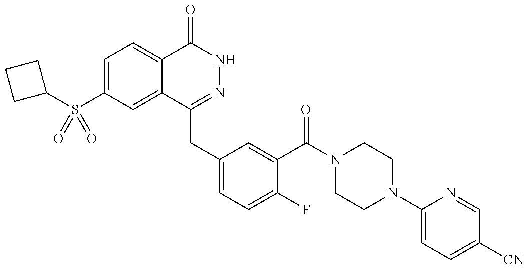 [SMILES: N#Cc1ccc(N2CCN(C(=O)c3cc(Cc4n[nH]c(=O)c5ccc(S(=O)(=O)C6CCC6)cc45)ccc3F)CC2)nc1] |
| 54 | 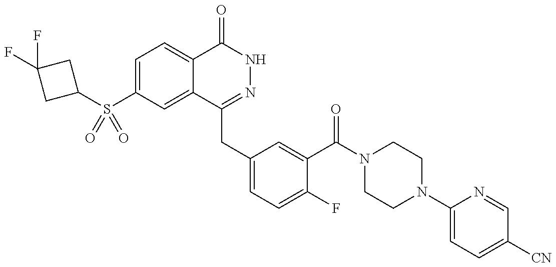 [SMILES: N#Cc1ccc(N2CCN(C(=O)c3cc(Cc4n[nH]c(=O)c5ccc(S(=O)(=O)C6CC(F)(F)C6)cc45)ccc3F)CC2)nc1] |
| 55 | 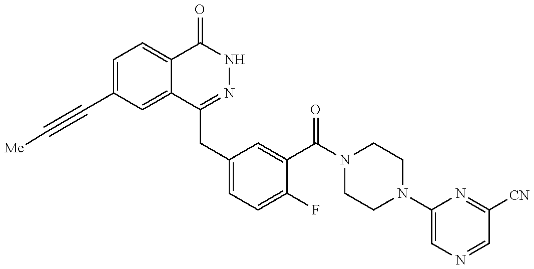 [SMILES: CC#Cc1ccc2c(=O)[nH]nc(Cc3ccc(F)c(C(=O)N4CCN(c5cncc(C#N)n5)CC4)c3)c2c1] |
| 56 | 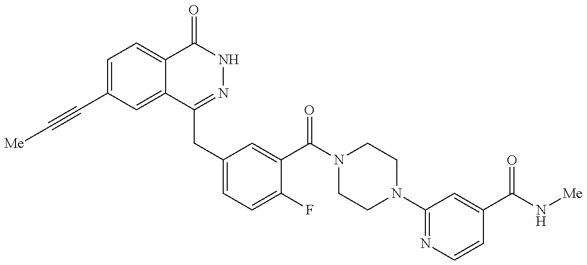 [SMILES: CC#Cc1ccc2c(=O)[nH]nc(Cc3ccc(F)c(C(=O)N4CCN(c5cc(C(=O)NC)ccn5)CC4)c3)c2c1] |
| 57 | 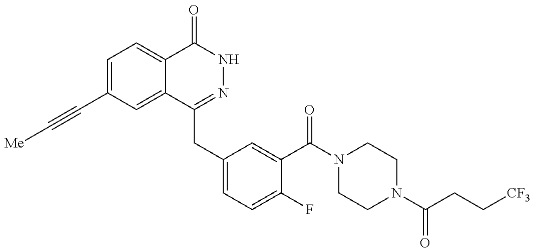 [SMILES: CC#Cc1ccc2c(=O)[nH]nc(Cc3ccc(F)c(C(=O)N4CCN(C(=O)CCC(F)(F)F)CC4)c3)c2c1] |
| 58 | 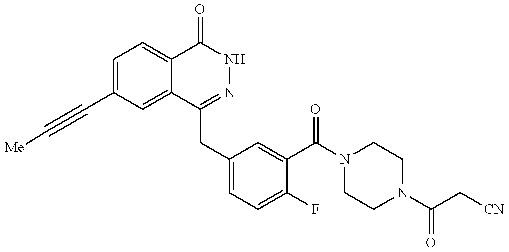 [SMILES: CC#Cc1ccc2c(=O)[nH]nc(Cc3ccc(F)c(C(=O)N4CCN(C(=O)CC#N)CC4)c3)c2c1] |
| 59 | 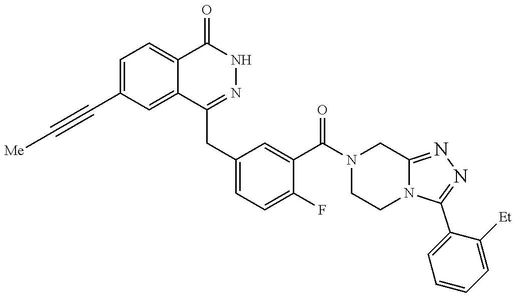 [SMILES: CC#Cc1ccc2c(=O)[nH]nc(Cc3ccc(F)c(C(=O)N4CCn5c(nnc5-c5ccccc5CC)C4)c3)c2c1] |
| 60 | 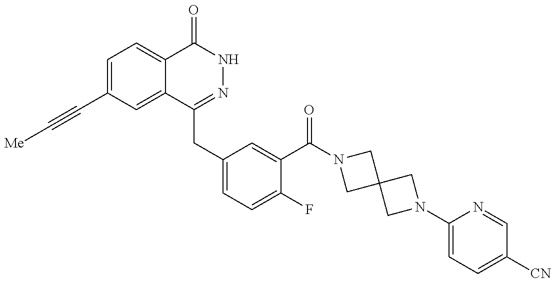 [SMILES: CC#Cc1ccc2c(=O)[nH]nc(Cc3ccc(F)c(C(=O)N4CC5(C4)CN(c4ccc(C#N)cn4)C5)c3)c2c1] |
| 61 | 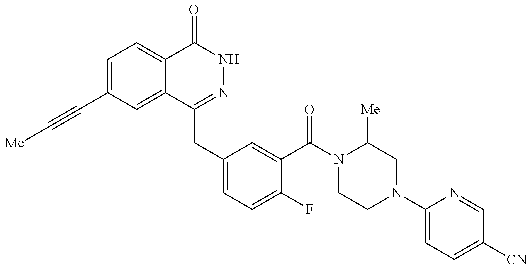 [SMILES: CC#Cc1ccc2c(=O)[nH]nc(Cc3ccc(F)c(C(=O)N4CCN(c5ccc(C#N)cn5)CC4C)c3)c2c1] |
| 62 | 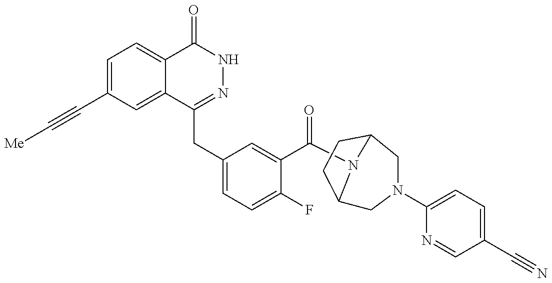 [SMILES: CC#Cc1ccc2c(=O)[nH]nc(Cc3ccc(F)c(C(=O)N4C5CCC4CN(c4ccc(C#N)cn4)C5)c3)c2c1] |
| 63 | 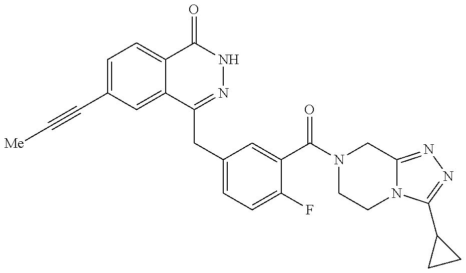 [SMILES: CC#Cc1ccc2c(=O)[nH]nc(Cc3ccc(F)c(C(=O)N4CCn5c(nnc5C5CC5)C4)c3)c2c1] |
| 64 | 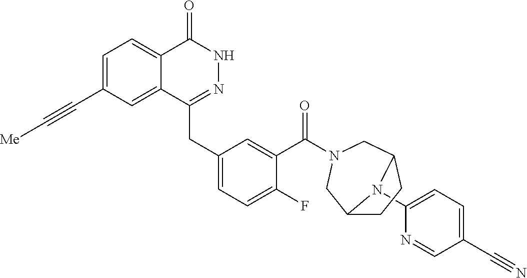 [SMILES: CC#Cc1ccc2c(=O)[nH]nc(Cc3ccc(F)c(C(=O)N4CC5CCC(C4)N5c4ccc(C#N)cn4)c3)c2c1] |
| 65 | 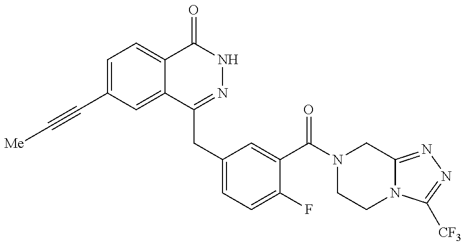 [SMILES: CC#Cc1ccc2c(=O)[nH]nc(Cc3ccc(F)c(C(=O)N4CCn5c(nnc5C(F)(F)F)C4)c3)c2c1] |
| 66 | 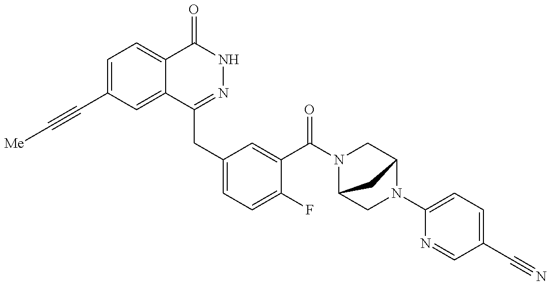 [SMILES: CC#Cc1ccc2c(=O)[nH]nc(Cc3ccc(F)c(C(=O)N4C[C@H]5C[C@@H]4CN5c4ccc(C#N)cn4)c3)c2c1] |
| 67 | 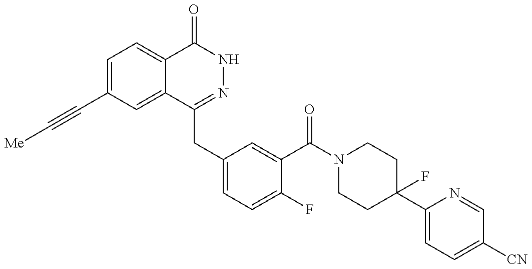 [SMILES: CC#Cc1ccc2c(=O)[nH]nc(Cc3ccc(F)c(C(=O)N4CCC(F)(c5ccc(C#N)cn5)CC4)c3)c2c1] |
| 68 | 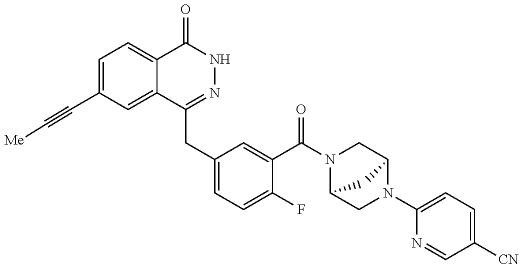 [SMILES: CC#Cc1ccc2c(=O)[nH]nc(Cc3ccc(F)c(C(=O)N4C[C@@H]5C[C@H]4CN5c4ccc(C#N)cn4)c3)c2c1] |
| 69 | 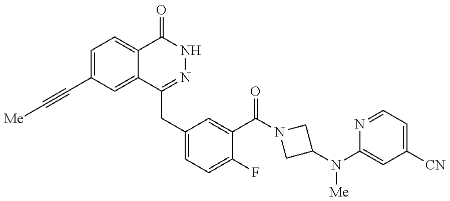 [SMILES: CC#Cc1ccc2c(=O)[nH]nc(Cc3ccc(F)c(C(=O)N4CC(N(C)c5cc(C#N)ccn5)C4)c3)c2c1] |
| 70 | 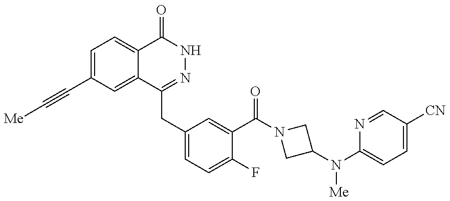 [SMILES: CC#Cc1ccc2c(=O)[nH]nc(Cc3ccc(F)c(C(=O)N4CC(N(C)c5ccc(C#N)cn5)C4)c3)c2c1] |
| 71 | 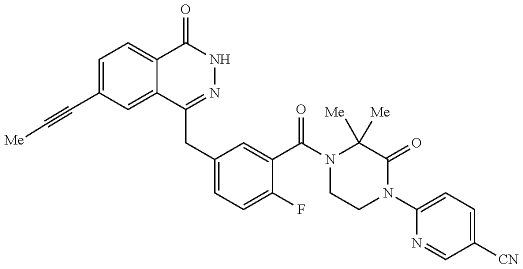 [SMILES: CC#Cc1ccc2c(=O)[nH]nc(Cc3ccc(F)c(C(=O)N4CCN(c5ccc(C#N)cn5)C(=O)C4(C)C)c3)c2c1] |
| 72 | 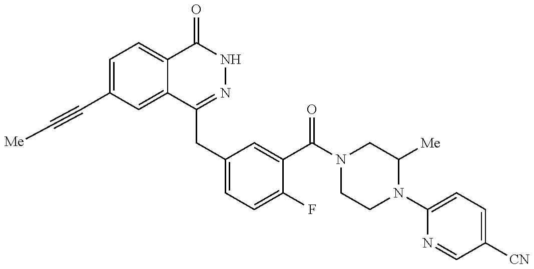 [SMILES: CC#Cc1ccc2c(=O)[nH]nc(Cc3ccc(F)c(C(=O)N4CCN(c5ccc(C#N)cn5)C(C)C4)c3)c2c1] |
| 73 | 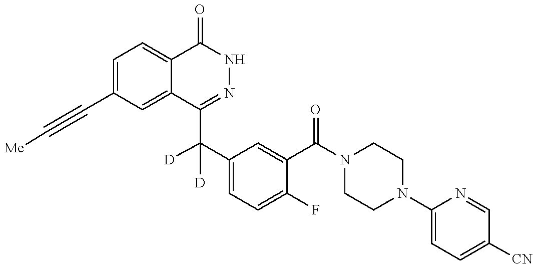 [SMILES: [2H]C([2H])(c1ccc(F)c(C(=O)N2CCN(c3ccc(C#N)cn3)CC2)c1)c1n[nH]c(=O)c2ccc(C#CC)cc12] |
| 74 | 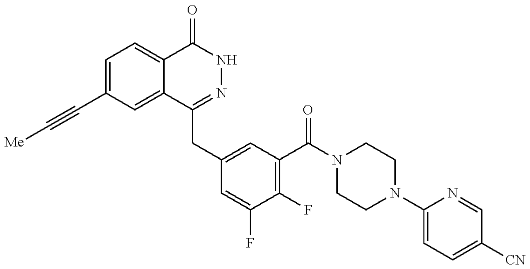 [SMILES: CC#Cc1ccc2c(=O)[nH]nc(Cc3cc(F)c(F)c(C(=O)N4CCN(c5ccc(C#N)cn5)CC4)c3)c2c1] |
| 75 | 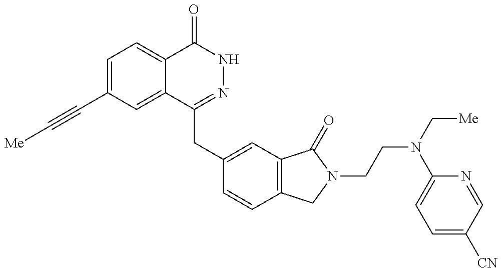 [SMILES: CC#Cc1ccc2c(=O)[nH]nc(Cc3ccc4c(c3)C(=O)N(CCN(CC)c3ccc(C#N)cn3)C4)c2c1] |
| 76 | 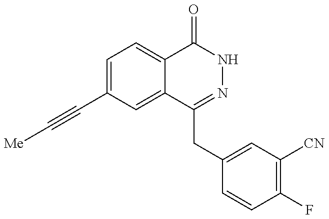 [SMILES: CC#Cc1ccc2c(=O)[nH]nc(Cc3ccc(F)c(C#N)c3)c2c1] |
| 77 | 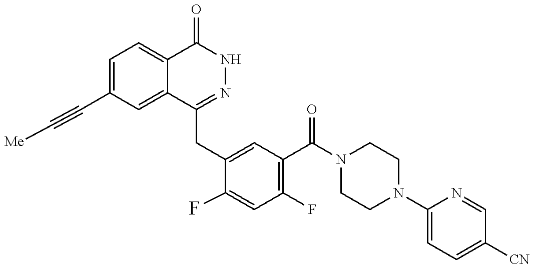 [SMILES: CC#Cc1ccc2c(=O)[nH]nc(Cc3cc(C(=O)N4CCN(c5ccc(C#N)cn5)CC4)c(F)cc3F)c2c1] |
| 78 | 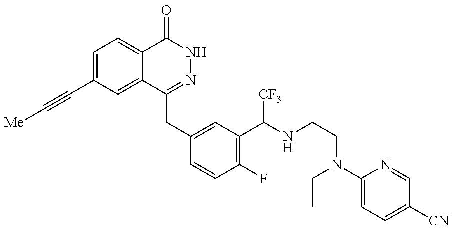 [SMILES: CC#Cc1ccc2c(=O)[nH]nc(Cc3ccc(F)c(C(NCCN(CC)c4ccc(C#N)cn4)C(F)(F)F)c3)c2c1] |
| 79 | 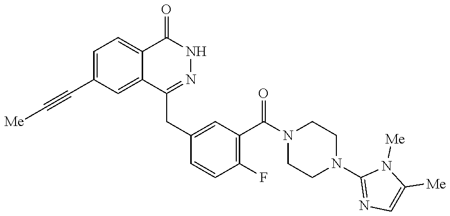 [SMILES: CC#Cc1ccc2c(=O)[nH]nc(Cc3ccc(F)c(C(=O)N4CCN(c5ncc(C)n5C)CC4)c3)c2c1] |
| 80 | 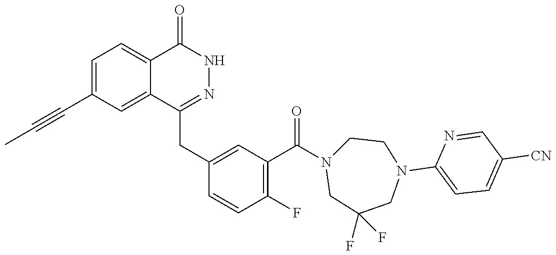 [SMILES: CC#Cc1ccc2c(=O)[nH]nc(Cc3ccc(F)c(C(=O)N4CCN(c5ccc(C#N)cn5)CC(F)(F)C4)c3)c2c1] |
| 81 | 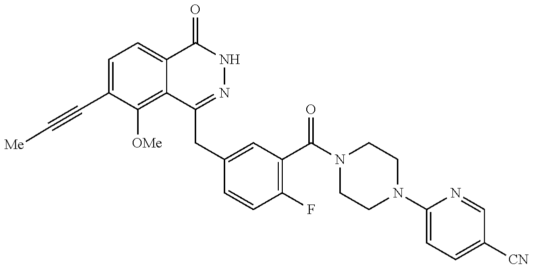 [SMILES: CC#Cc1ccc2c(=O)[nH]nc(Cc3ccc(F)c(C(=O)N4CCN(c5ccc(C#N)cn5)CC4)c3)c2c1OC] |
| 82 | 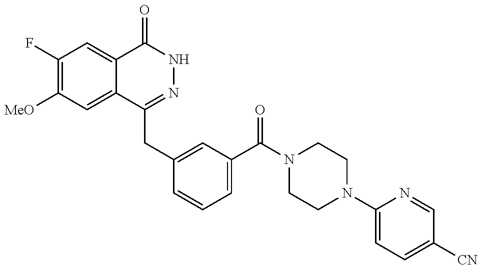 [SMILES: COc1cc2c(Cc3cccc(C(=O)N4CCN(c5ccc(C#N)cn5)CC4)c3)n[nH]c(=O)c2cc1F] |
| 83 | 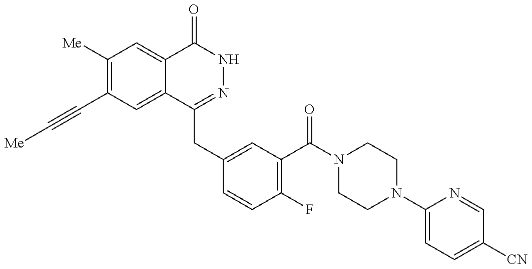 [SMILES: CC#Cc1cc2c(Cc3ccc(F)c(C(=O)N4CCN(c5ccc(C#N)cn5)CC4)c3)n[nH]c(=O)c2cc1C] |
| 84 | 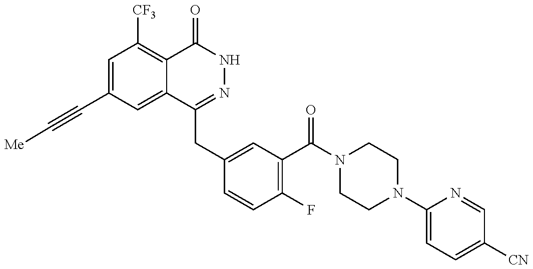 [SMILES: CC#Cc1cc(C(F)(F)F)c2c(=O)[nH]nc(Cc3ccc(F)c(C(=O)N4CCN(c5ccc(C#N)cn5)CC4)c3)c2c1] |
| 85 | 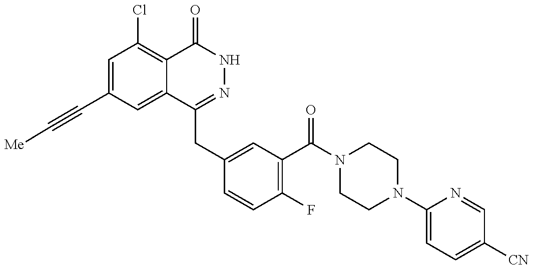 [SMILES: CC#Cc1cc(Cl)c2c(=O)[nH]nc(Cc3ccc(F)c(C(=O)N4CCN(c5ccc(C#N)cn5)CC4)c3)c2c1] |
| 86 | 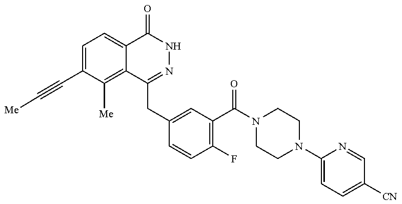 |
| 87 | 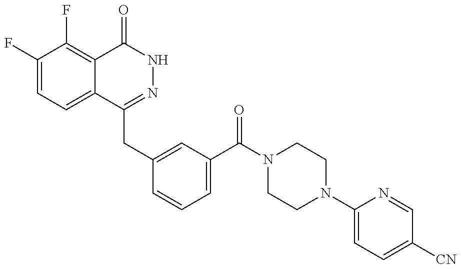 [SMILES: N#Cc1ccc(N2CCN(C(=O)c3cccc(Cc4n[nH]c(=O)c5c(F)c(F)ccc45)c3)CC2)nc1] |
| 88 | 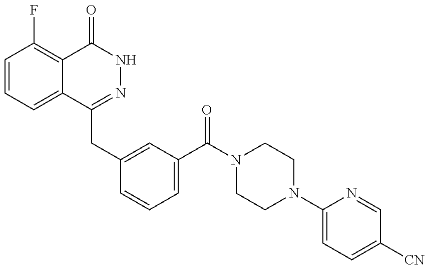 [SMILES: N#Cc1ccc(N2CCN(C(=O)c3cccc(Cc4n[nH]c(=O)c5c(F)cccc45)c3)CC2)nc1] |
| 89 | 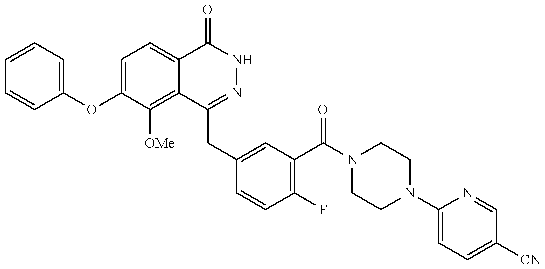 [SMILES: COc1c(Oc2ccccc2)ccc2c(=O)[nH]nc(Cc3ccc(F)c(C(=O)N4CCN(c5ccc(C#N)cn5)CC4)c3)c12] |
| 90 | 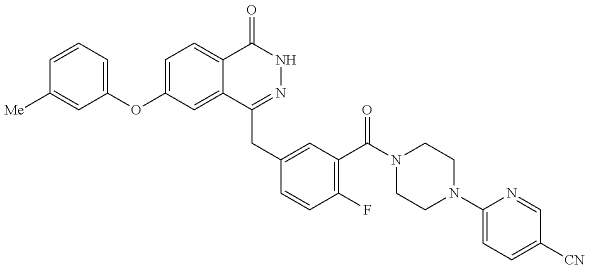 [SMILES: Cc1cccc(Oc2ccc3c(=O)[nH]nc(Cc4ccc(F)c(C(=O)N5CCN(c6ccc(C#N)cn6)CC5)c4)c3c2)c1] |
| 91 | 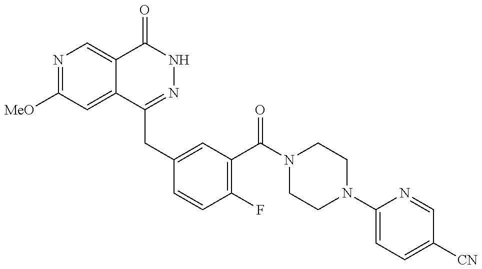 [SMILES: COc1cc2c(Cc3ccc(F)c(C(=O)N4CCN(c5ccc(C#N)cn5)CC4)c3)n[nH]c(=O)c2cn1] |
| 92 | 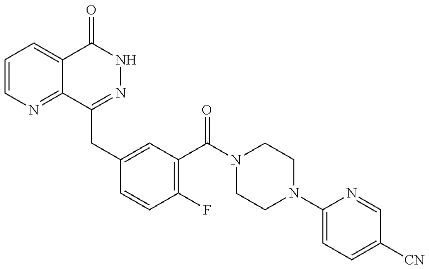 [SMILES: N#Cc1ccc(N2CCN(C(=O)c3cc(Cc4n[nH]c(=O)c5cccnc45)ccc3F)CC2)nc1] |
| 93 | 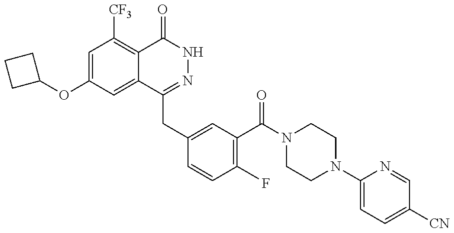 [SMILES: N#Cc1ccc(N2CCN(C(=O)c3cc(Cc4n[nH]c(=O)c5c(C(F)(F)F)cc(OC6CCC6)cc45)ccc3F)CC2)nc1] |
| 94 | 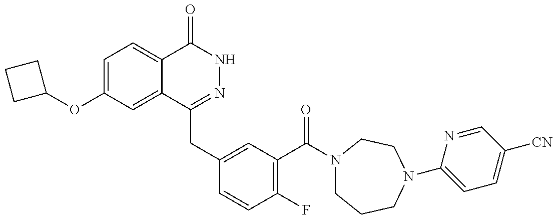 [SMILES: N#Cc1ccc(N2CCCN(C(=O)c3cc(Cc4n[nH]c(=O)c5ccc(OC6CCC6)cc45)ccc3F)CC2)nc1] |
| 95 | 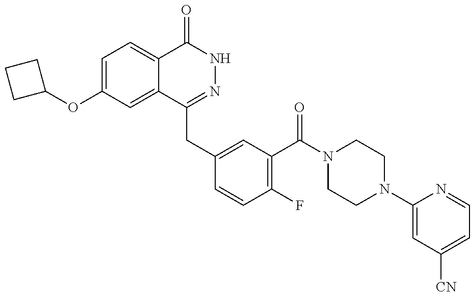 [SMILES: N#Cc1ccnc(N2CCN(C(=O)c3cc(Cc4n[nH]c(=O)c5ccc(OC6CCC6)cc45)ccc3F)CC2)c1] |
| 96 | 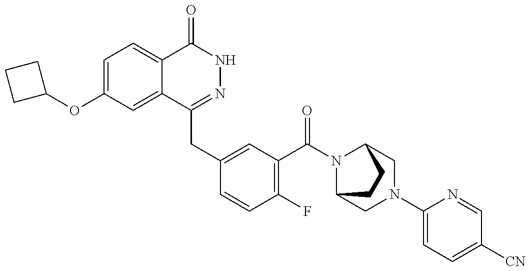 |
| 97 | 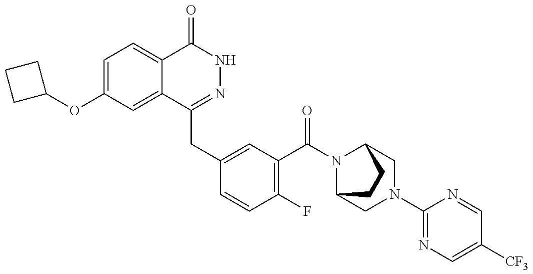 |
| 98 | 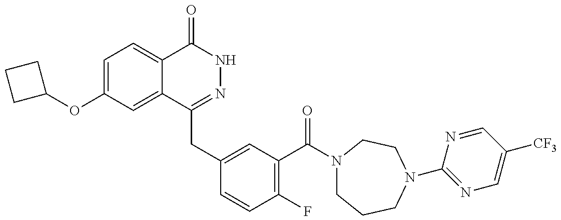 [SMILES: O=C(c1cc(Cc2n[nH]c(=O)c3ccc(OC4CCC4)cc23)ccc1F)N1CCCN(c2ncc(C(F)(F)F)cn2)CC1] |
| 99 | 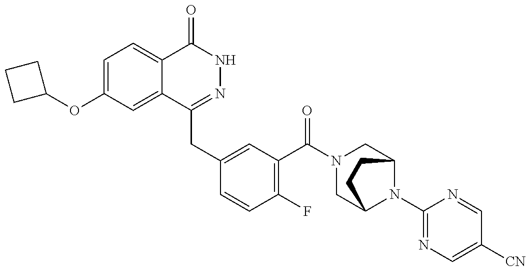 |
| 100 | 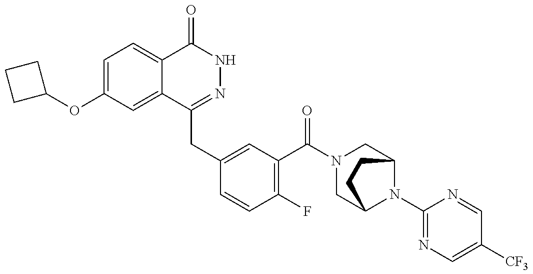 |
| 101 | 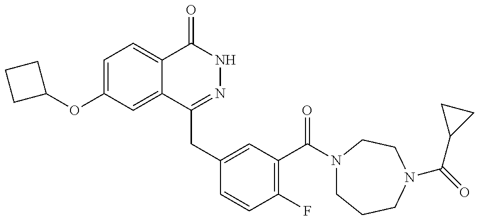 [SMILES: O=C(c1cc(Cc2n[nH]c(=O)c3ccc(OC4CCC4)cc23)ccc1F)N1CCCN(C(=O)C2CC2)CC1] |
| 102 | 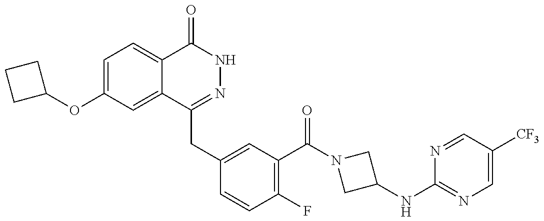 [SMILES: O=C(c1cc(Cc2n[nH]c(=O)c3ccc(OC4CCC4)cc23)ccc1F)N1CC(Nc2ncc(C(F)(F)F)cn2)C1] |
| 103 | 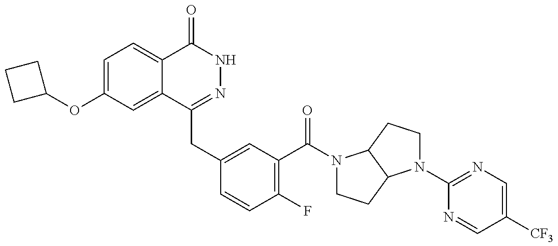 [SMILES: O=C(c1cc(Cc2n[nH]c(=O)c3ccc(OC4CCC4)cc23)ccc1F)N1CCC2C1CCN2c1ncc(C(F)(F)F)cn1] |
| 104 | 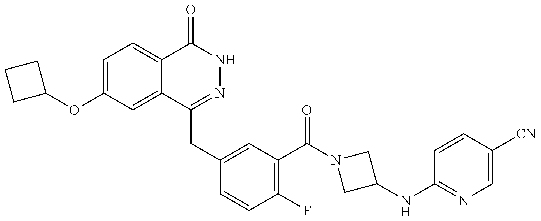 [SMILES: N#Cc1ccc(NC2CN(C(=O)c3cc(Cc4n[nH]c(=O)c5ccc(OC6CCC6)cc45)ccc3F)C2)nc1] |
| 105 | 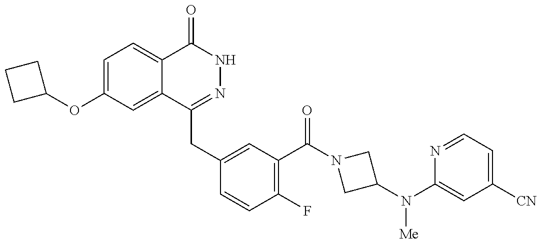 [SMILES: CN(c1cc(C#N)ccn1)C1CN(C(=O)c2cc(Cc3n[nH]c(=O)c4ccc(OC5CCC5)cc34)ccc2F)C1] |
| 106 | 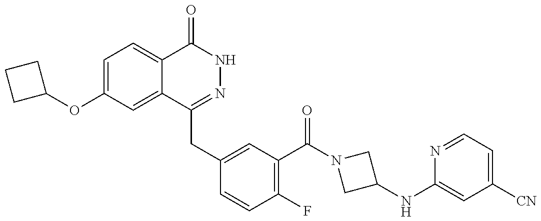 [SMILES: N#Cc1ccnc(NC2CN(C(=O)c3cc(Cc4n[nH]c(=O)c5ccc(OC6CCC6)cc45)ccc3F)C2)c1] |
| 107 | 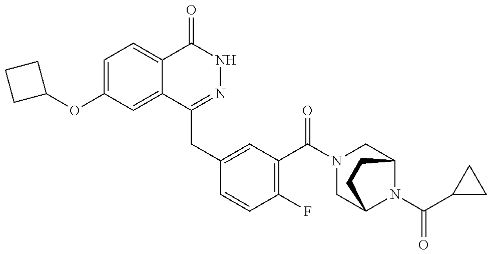 |
| 108 | 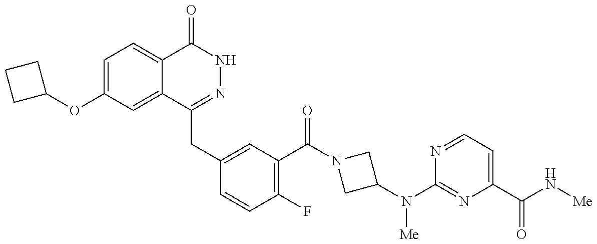 [SMILES: CNC(=O)c1ccnc(N(C)C2CN(C(=O)c3cc(Cc4n[nH]c(=O)c5ccc(OC6CCC6)cc45)ccc3F)C2)n1] |
| 109 | 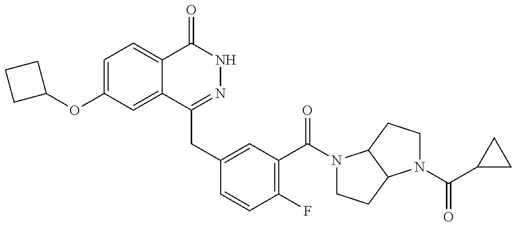 [SMILES: O=C(c1cc(Cc2n[nH]c(=O)c3ccc(OC4CCC4)cc23)ccc1F)N1CCC2C1CCN2C(=O)C1CC1] |
| 110 | 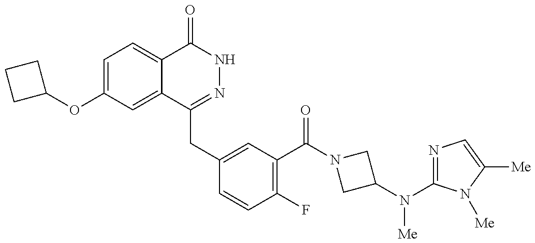 [SMILES: Cc1cnc(N(C)C2CN(C(=O)c3cc(Cc4n[nH]c(=O)c5ccc(OC6CCC6)cc45)ccc3F)C2)n1C] |
| 111 | 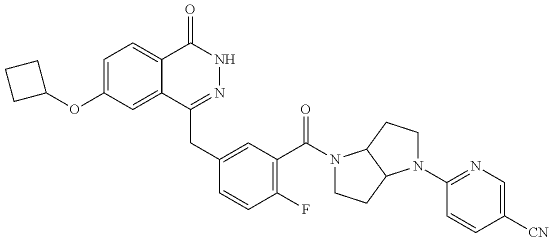 [SMILES: N#Cc1ccc(N2CCC3C2CCN3C(=O)c2cc(Cc3n[nH]c(=O)c4ccc(OC5CCC5)cc34)ccc2F)nc1] |
| 112 | 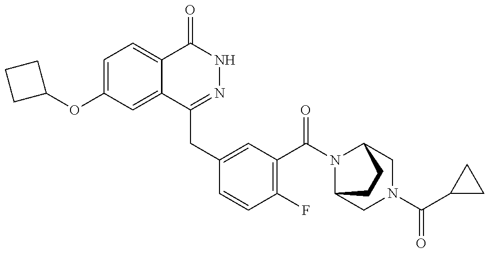 |
| 113 | 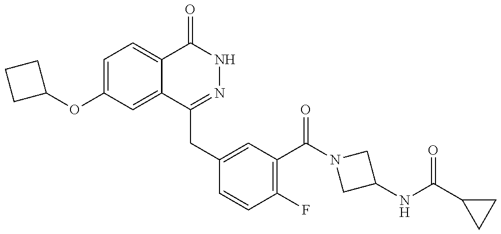 [SMILES: O=C(NC1CN(C(=O)c2cc(Cc3n[nH]c(=O)c4ccc(OC5CCC5)cc34)ccc2F)C1)C1CC1] |
| 114 | 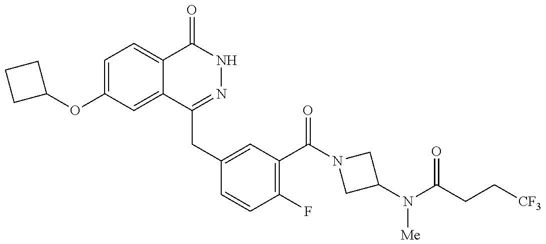 [SMILES: CN(C(=O)CCC(F)(F)F)C1CN(C(=O)c2cc(Cc3n[nH]c(=O)c4ccc(OC5CCC5)cc34)ccc2F)C1] |
| 115 | 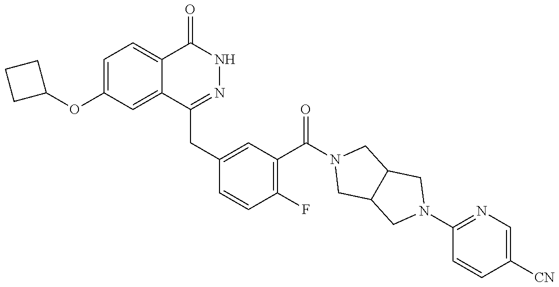 [SMILES: N#Cc1ccc(N2CC3CN(C(=O)c4cc(Cc5n[nH]c(=O)c6ccc(OC7CCC7)cc56)ccc4F)CC3C2)nc1] |
| 116 | 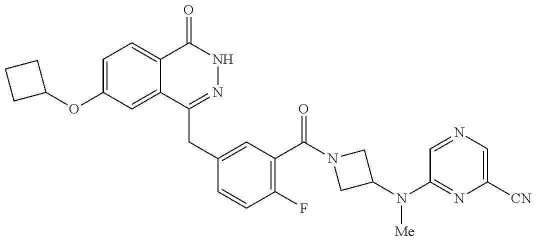 [SMILES: CN(c1cncc(C#N)n1)C1CN(C(=O)c2cc(Cc3n[nH]c(=O)c4ccc(OC5CCC5)cc34)ccc2F)C1] |
| 117 | 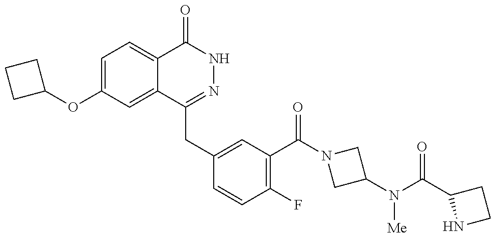 |
| 118 | 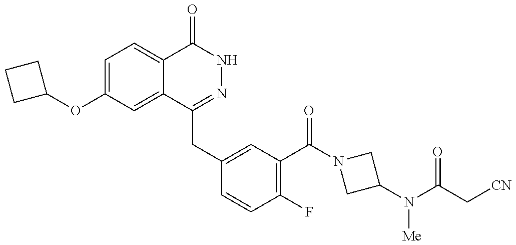 [SMILES: CN(C(=O)CC#N)C1CN(C(=O)c2cc(Cc3n[nH]c(=O)c4ccc(OC5CCC5)cc34)ccc2F)C1] |
| 119 | 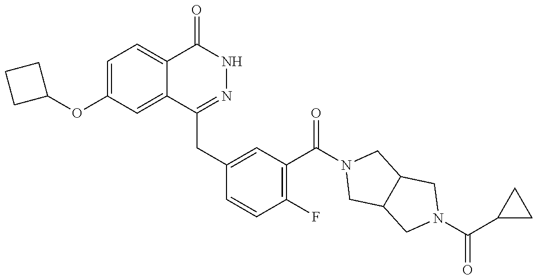 [SMILES: O=C(c1cc(Cc2n[nH]c(=O)c3ccc(OC4CCC4)cc23)ccc1F)N1CC2CN(C(=O)C3CC3)CC2C1] |
| 120 | 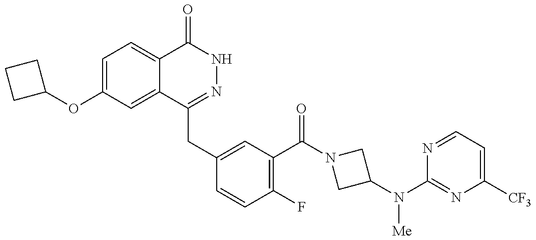 [SMILES: CN(c1nccc(C(F)(F)F)n1)C1CN(C(=O)c2cc(Cc3n[nH]c(=O)c4ccc(OC5CCC5)cc34)ccc2F)C1] |
| 121 | 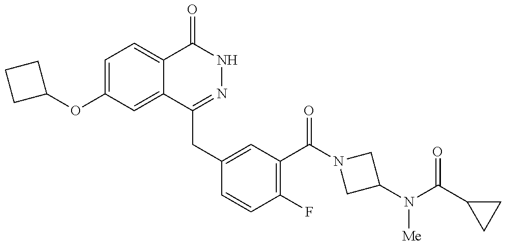 [SMILES: CN(C(=O)C1CC1)C1CN(C(=O)c2cc(Cc3n[nH]c(=O)c4ccc(OC5CCC5)cc34)ccc2F)C1] |
| 122 | 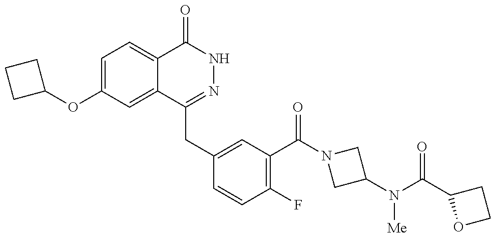 |
| 123 | 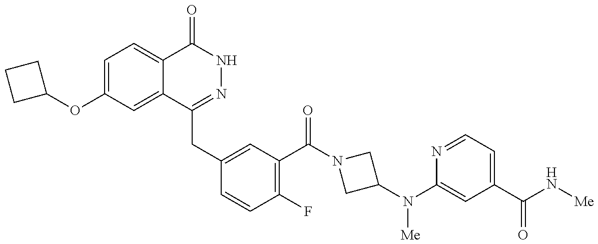 [SMILES: CNC(=O)c1ccnc(N(C)C2CN(C(=O)c3cc(Cc4n[nH]c(=O)c5ccc(OC6CCC6)cc45)ccc3F)C2)c1] |
| 124 | 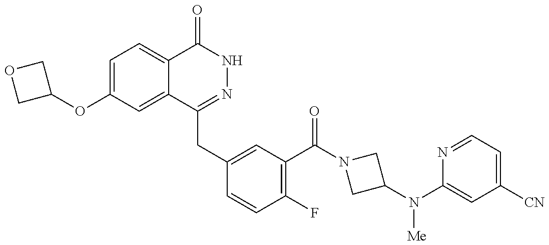 [SMILES: CN(c1cc(C#N)ccn1)C1CN(C(=O)c2cc(Cc3n[nH]c(=O)c4ccc(OC5COC5)cc34)ccc2F)C1] |
| 125 | 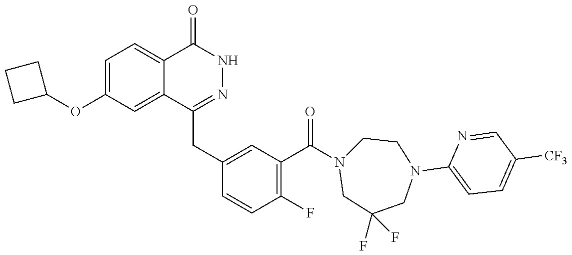 [SMILES: O=C(c1cc(Cc2n[nH]c(=O)c3ccc(OC4CCC4)cc23)ccc1F)N1CCN(c2ccc(C(F)(F)F)cn2)CC(F)(F)C1] |
| 126 | 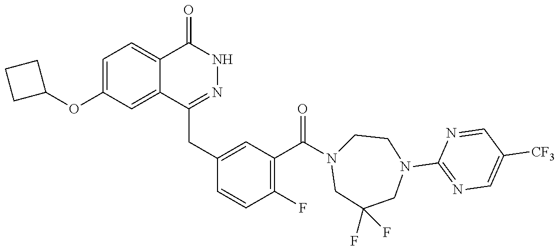 [SMILES: O=C(c1cc(Cc2n[nH]c(=O)c3ccc(OC4CCC4)cc23)ccc1F)N1CCN(c2ncc(C(F)(F)F)cn2)CC(F)(F)C1] |
| 127 | 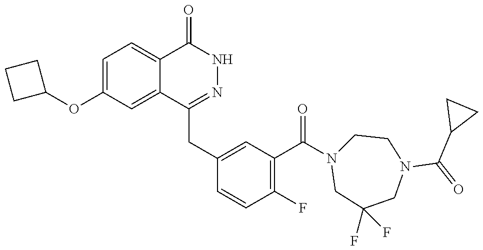 [SMILES: O=C(c1cc(Cc2n[nH]c(=O)c3ccc(OC4CCC4)cc23)ccc1F)N1CCN(C(=O)C2CC2)CC(F)(F)C1] |
| 128 | 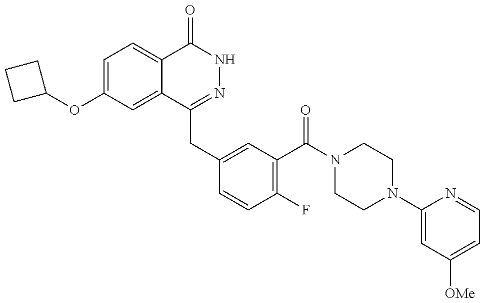 [SMILES: COc1ccnc(N2CCN(C(=O)c3cc(Cc4n[nH]c(=O)c5ccc(OC6CCC6)cc45)ccc3F)CC2)c1] |
| 129 | 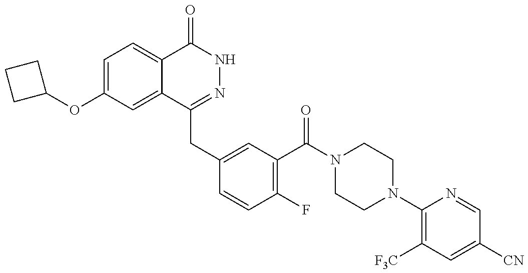 [SMILES: N#Cc1cnc(N2CCN(C(=O)c3cc(Cc4n[nH]c(=O)c5ccc(OC6CCC6)cc45)ccc3F)CC2)c(C(F)(F)F)c1] |
| 130 | 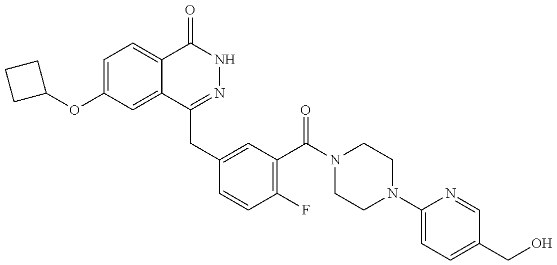 [SMILES: O=C(c1cc(Cc2n[nH]c(=O)c3ccc(OC4CCC4)cc23)ccc1F)N1CCN(c2ccc(CO)cn2)CC1] |
| 131 | 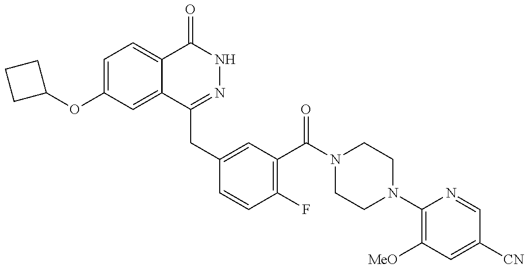 [SMILES: COc1cc(C#N)cnc1N1CCN(C(=O)c2cc(Cc3n[nH]c(=O)c4ccc(OC5CCC5)cc34)ccc2F)CC1] |
| 132 | 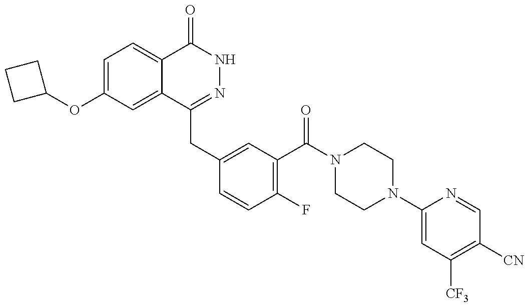 [SMILES: N#Cc1cnc(N2CCN(C(=O)c3cc(Cc4n[nH]c(=O)c5ccc(OC6CCC6)cc45)ccc3F)CC2)cc1C(F)(F)F] |
| 133 | 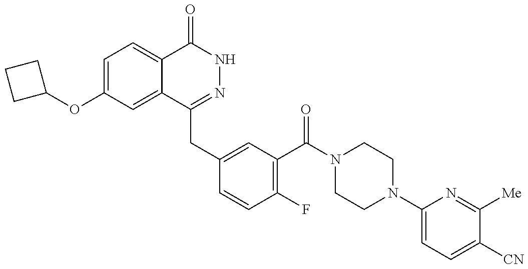 [SMILES: Cc1nc(N2CCN(C(=O)c3cc(Cc4n[nH]c(=O)c5ccc(OC6CCC6)cc45)ccc3F)CC2)ccc1C#N] |
| 134 | 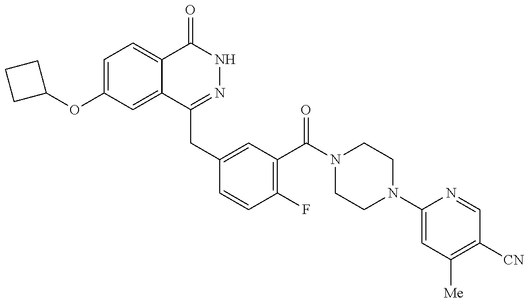 [SMILES: Cc1cc(N2CCN(C(=O)c3cc(Cc4n[nH]c(=O)c5ccc(OC6CCC6)cc45)ccc3F)CC2)ncc1C#N] |
| 135 | 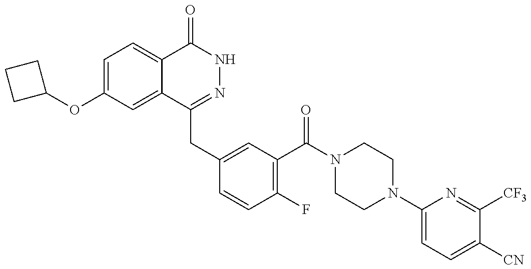 [SMILES: N#Cc1ccc(N2CCN(C(=O)c3cc(Cc4n[nH]c(=O)c5ccc(OC6CCC6)cc45)ccc3F)CC2)nc1C(F)(F)F] |
| 136 | 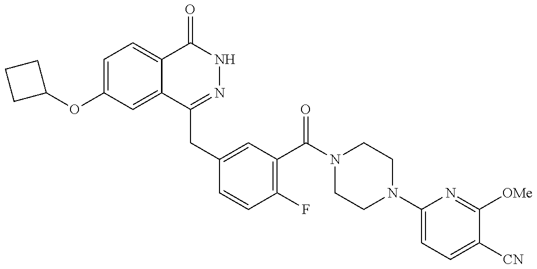 [SMILES: COc1nc(N2CCN(C(=O)c3cc(Cc4n[nH]c(=O)c5ccc(OC6CCC6)cc45)ccc3F)CC2)ccc1C#N] |
| 137 | 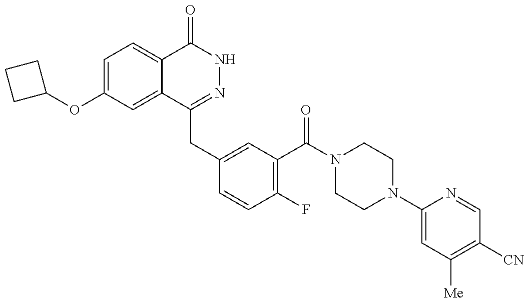 [SMILES: Cc1cc(N2CCN(C(=O)c3cc(Cc4n[nH]c(=O)c5ccc(OC6CCC6)cc45)ccc3F)CC2)ncc1C#N] |
| 138 | 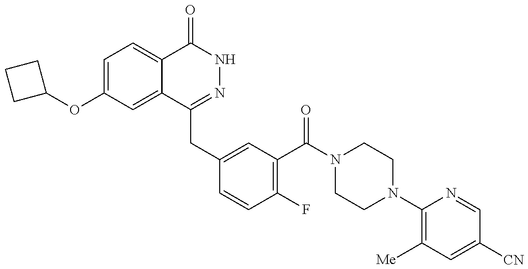 [SMILES: Cc1cc(C#N)cnc1N1CCN(C(=O)c2cc(Cc3n[nH]c(=O)c4ccc(OC5CCC5)cc34)ccc2F)CC1] |
| 139 | 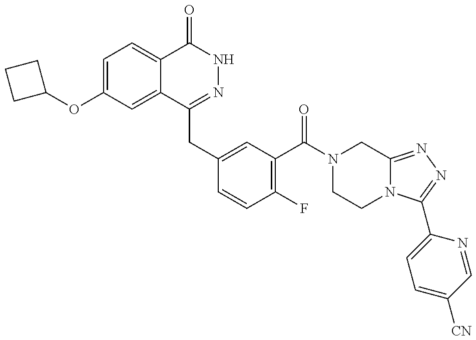 [SMILES: N#Cc1ccc(-c2nnc3n2CCN(C(=O)c2cc(Cc4n[nH]c(=O)c5ccc(OC6CCC6)cc45)ccc2F)C3)nc1] |
| 140 | 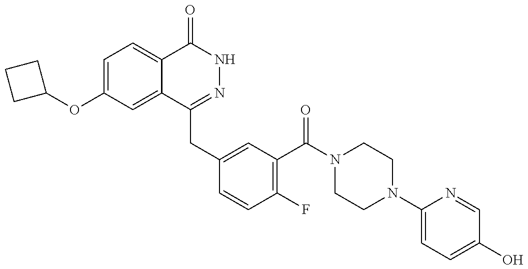 [SMILES: O=C(c1cc(Cc2n[nH]c(=O)c3ccc(OC4CCC4)cc23)ccc1F)N1CCN(c2ccc(O)cn2)CC1] |
| 141 | 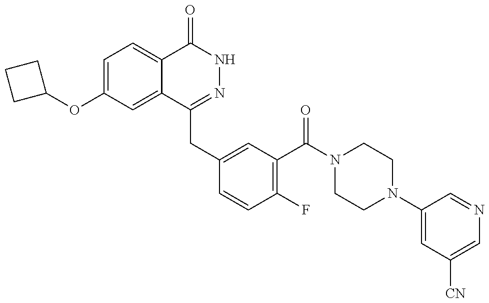 [SMILES: N#Cc1cncc(N2CCN(C(=O)c3cc(Cc4n[nH]c(=O)c5ccc(OC6CCC6)cc45)ccc3F)CC2)c1] |
| 142 | 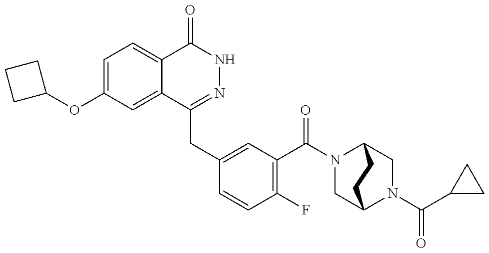 |
| 143 | 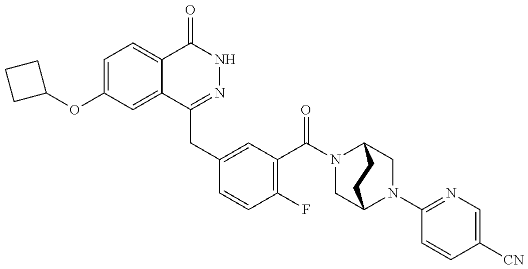 |
| 144 | 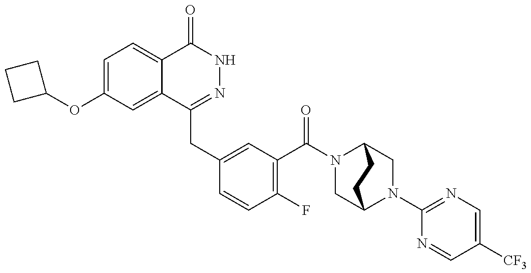 |
| 145 | 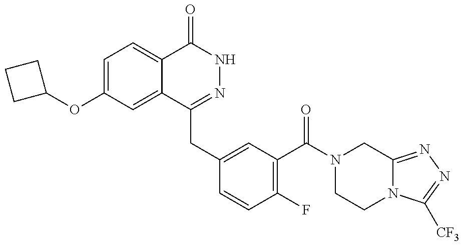 [SMILES: O=C(c1cc(Cc2n[nH]c(=O)c3ccc(OC4CCC4)cc23)ccc1F)N1CCn2c(nnc2C(F)(F)F)C1] |
| 146 | 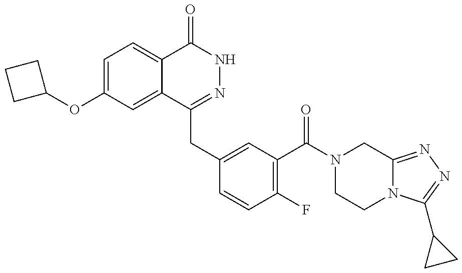 [SMILES: O=C(c1cc(Cc2n[nH]c(=O)c3ccc(OC4CCC4)cc23)ccc1F)N1CCn2c(nnc2C2CC2)C1] |
| 147 | 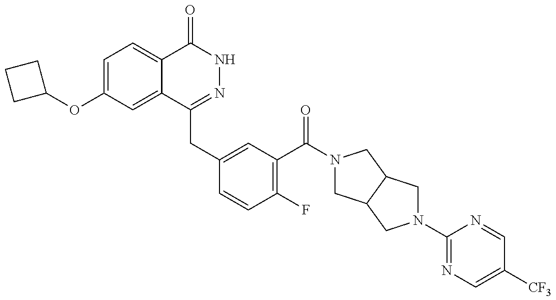 [SMILES: O=C(c1cc(Cc2n[nH]c(=O)c3ccc(OC4CCC4)cc23)ccc1F)N1CC2CN(c3ncc(C(F)(F)F)cn3)CC2C1] |
| 147.1 | 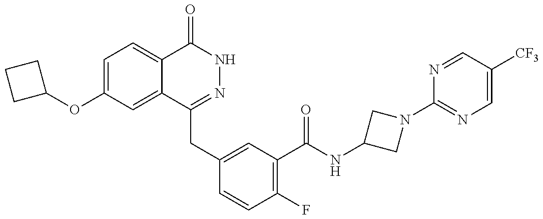 [SMILES: O=C(NC1CN(c2ncc(C(F)(F)F)cn2)C1)c1cc(Cc2n[nH]c(=O)c3ccc(OC4CCC4)cc23)ccc1F] |
| 148 | 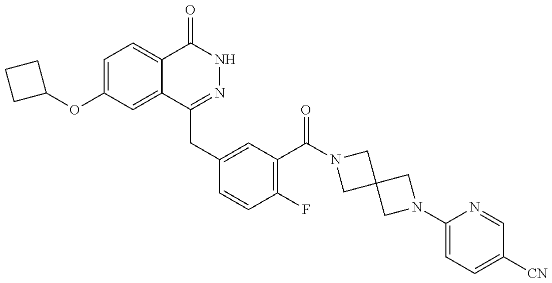 [SMILES: N#Cc1ccc(N2CC3(CN(C(=O)c4cc(Cc5n[nH]c(=O)c6ccc(OC7CCC7)cc56)ccc4F)C3)C2)nc1] |
| 149 | 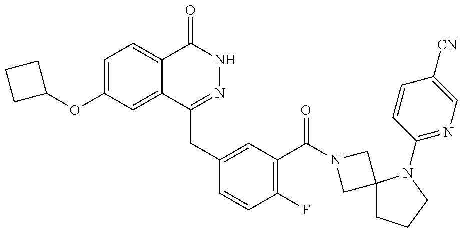 [SMILES: N#Cc1ccc(N2CCCC23CN(C(=O)c2cc(Cc4n[nH]c(=O)c5ccc(OC6CCC6)cc45)ccc2F)C3)nc1] |
| 150 | 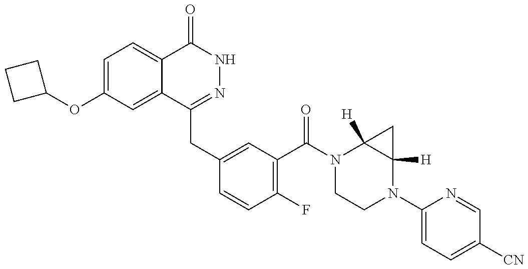 [SMILES: N#Cc1ccc(N2CCN(C(=O)c3cc(Cc4n[nH]c(=O)c5ccc(OC6CCC6)cc45)ccc3F)[C@@H]3C[C@@H]32)nc1] |
| 151 | 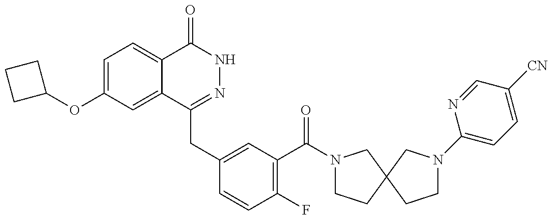 [SMILES: N#Cc1ccc(N2CCC3(CCN(C(=O)c4cc(Cc5n[nH]c(=O)c6ccc(OC7CCC7)cc56)ccc4F)C3)C2)nc1] |
| 152 | 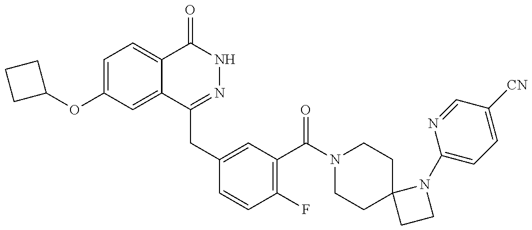 [SMILES: N#Cc1ccc(N2CCC23CCN(C(=O)c2cc(Cc4n[nH]c(=O)c5ccc(OC6CCC6)cc45)ccc2F)CC3)nc1] |
| 154 | 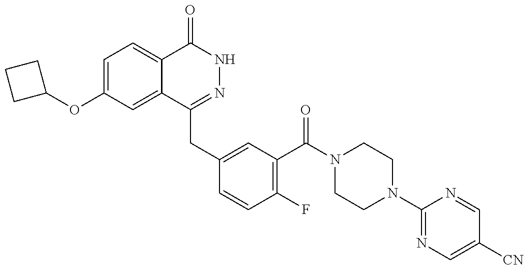 [SMILES: N#Cc1cnc(N2CCN(C(=O)c3cc(Cc4n[nH]c(=O)c5ccc(OC6CCC6)cc45)ccc3F)CC2)nc1] |
| 155 | 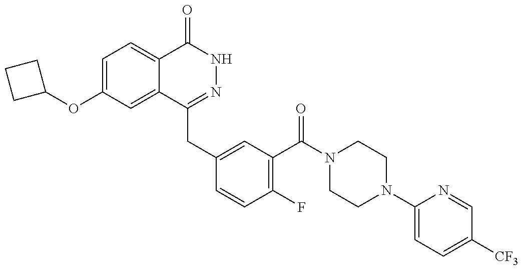 [SMILES: O=C(c1cc(Cc2n[nH]c(=O)c3ccc(OC4CCC4)cc23)ccc1F)N1CCN(c2ccc(C(F)(F)F)cn2)CC1] |
| 156 | 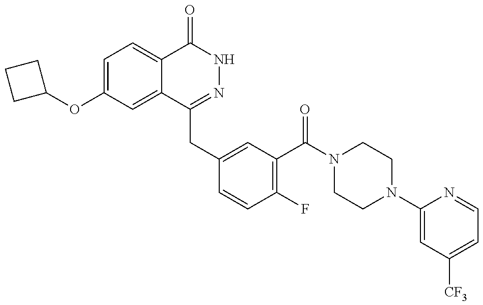 [SMILES: O=C(c1cc(Cc2n[nH]c(=O)c3ccc(OC4CCC4)cc23)ccc1F)N1CCN(c2cc(C(F)(F)F)ccn2)CC1] |
| 157 | 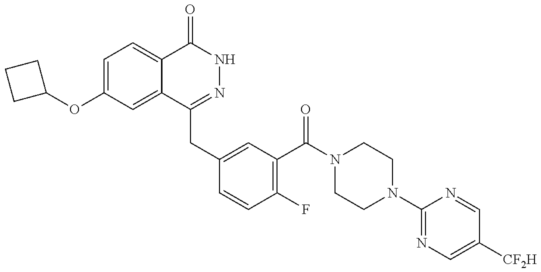 [SMILES: O=C(c1cc(Cc2n[nH]c(=O)c3ccc(OC4CCC4)cc23)ccc1F)N1CCN(c2ncc(C(F)F)cn2)CC1] |
| 158 | 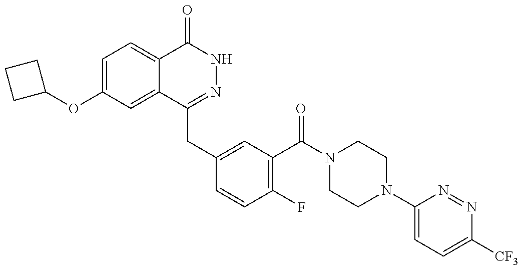 [SMILES: O=C(c1cc(Cc2n[nH]c(=O)c3ccc(OC4CCC4)cc23)ccc1F)N1CCN(c2ccc(C(F)(F)F)nn2)CC1] |
| 159 | 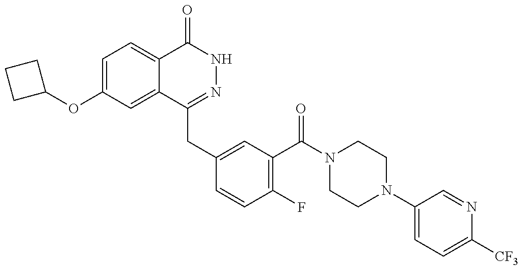 [SMILES: O=C(c1cc(Cc2n[nH]c(=O)c3ccc(OC4CCC4)cc23)ccc1F)N1CCN(c2ccc(C(F)(F)F)nc2)CC1] |
| 160 | 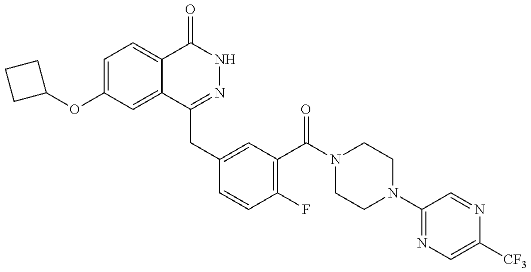 [SMILES: O=C(c1cc(Cc2n[nH]c(=O)c3ccc(OC4CCC4)cc23)ccc1F)N1CCN(c2cnc(C(F)(F)F)cn2)CC1] |
| 161 | 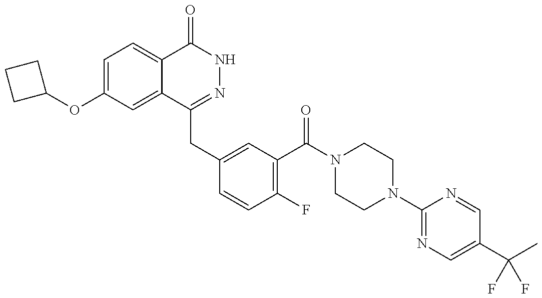 [SMILES: CC(F)(F)c1cnc(N2CCN(C(=O)c3cc(Cc4n[nH]c(=O)c5ccc(OC6CCC6)cc45)ccc3F)CC2)nc1] |
| 162 | 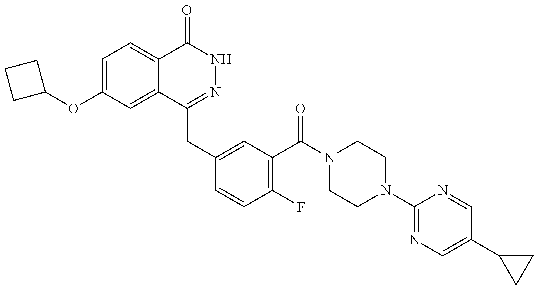 [SMILES: O=C(c1cc(Cc2n[nH]c(=O)c3ccc(OC4CCC4)cc23)ccc1F)N1CCN(c2ncc(C3CC3)cn2)CC1] |
| 163 | 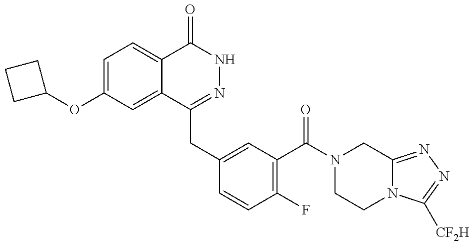 [SMILES: O=C(c1cc(Cc2n[nH]c(=O)c3ccc(OC4CCC4)cc23)ccc1F)N1CCn2c(nnc2C(F)F)C1] |
| 164 | 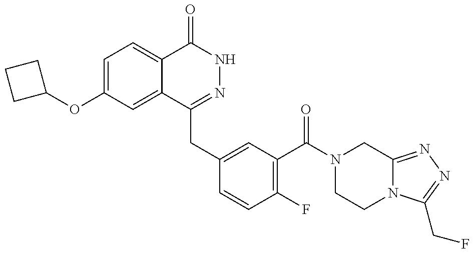 [SMILES: O=C(c1cc(Cc2n[nH]c(=O)c3ccc(OC4CCC4)cc23)ccc1F)N1CCn2c(CF)nnc2C1] |
| 165 | 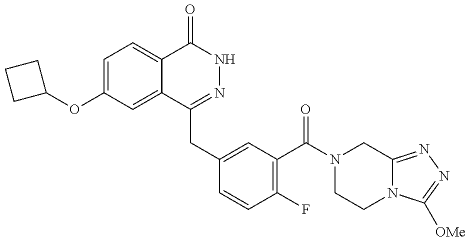 [SMILES: COc1nnc2n1CCN(C(=O)c1cc(Cc3n[nH]c(=O)c4ccc(OC5CCC5)cc34)ccc1F)C2] |
| 166 | 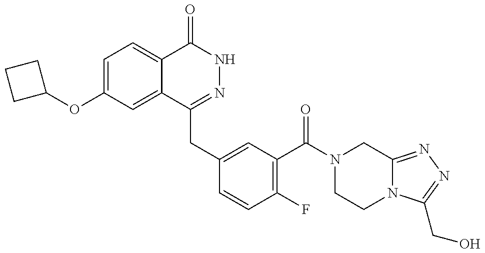 [SMILES: O=C(c1cc(Cc2n[nH]c(=O)c3ccc(OC4CCC4)cc23)ccc1F)N1CCn2c(CO)nnc2C1] |
| 167 | 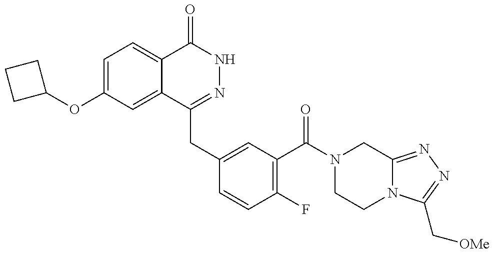 [SMILES: COCc1nnc2n1CCN(C(=O)c1cc(Cc3n[nH]c(=O)c4ccc(OC5CCC5)cc34)ccc1F)C2] |
| 168 | 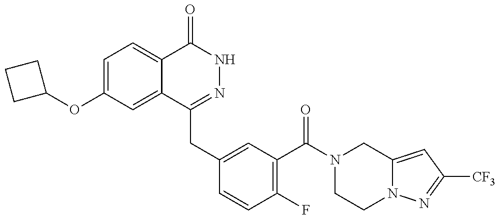 [SMILES: O=C(c1cc(Cc2n[nH]c(=O)c3ccc(OC4CCC4)cc23)ccc1F)N1CCn2nc(C(F)(F)F)cc2C1] |
| 169 | 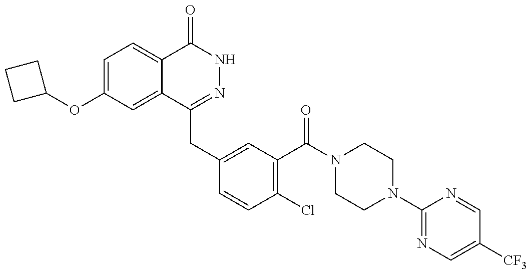 [SMILES: O=C(c1cc(Cc2n[nH]c(=O)c3ccc(OC4CCC4)cc23)ccc1Cl)N1CCN(c2ncc(C(F)(F)F)cn2)CC1] |
| 170 | 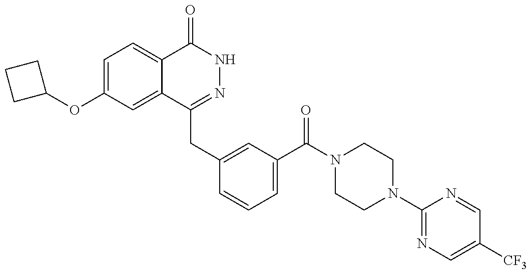 [SMILES: O=C(c1cccc(Cc2n[nH]c(=O)c3ccc(OC4CCC4)cc23)c1)N1CCN(c2ncc(C(F)(F)F)cn2)CC1] |
| 171 | 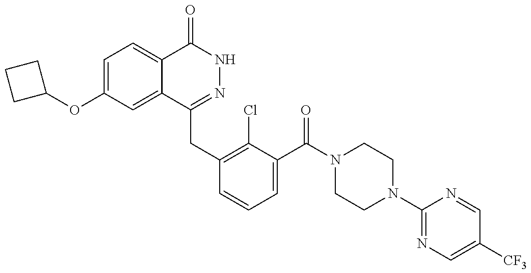 [SMILES: O=C(c1cccc(Cc2n[nH]c(=O)c3ccc(OC4CCC4)cc23)c1Cl)N1CCN(c2ncc(C(F)(F)F)cn2)CC1] |
| 172 | 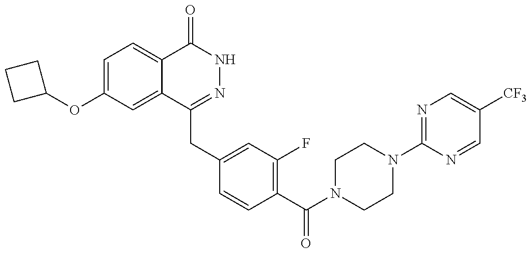 [SMILES: O=C(c1ccc(Cc2n[nH]c(=O)c3ccc(OC4CCC4)cc23)cc1F)N1CCN(c2ncc(C(F)(F)F)cn2)CC1] |
| 173 | 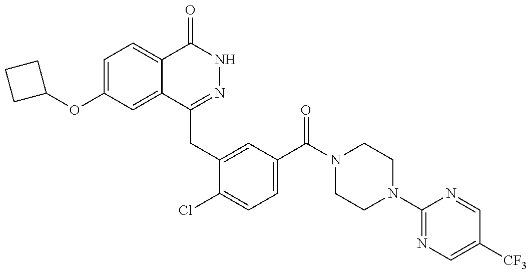 [SMILES: O=C(c1ccc(Cl)c(Cc2n[nH]c(=O)c3ccc(OC4CCC4)cc23)c1)N1CCN(c2ncc(C(F)(F)F)cn2)CC1] |
| 174 | 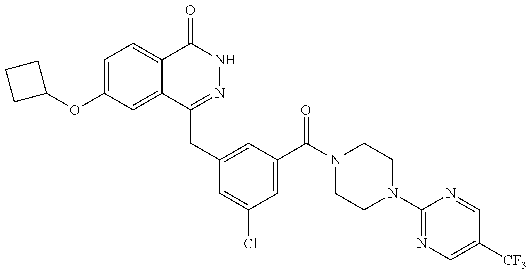 [SMILES: O=C(c1cc(Cl)cc(Cc2n[nH]c(=O)c3ccc(OC4CCC4)cc23)c1)N1CCN(c2ncc(C(F)(F)F)cn2)CC1] |
| 175 | 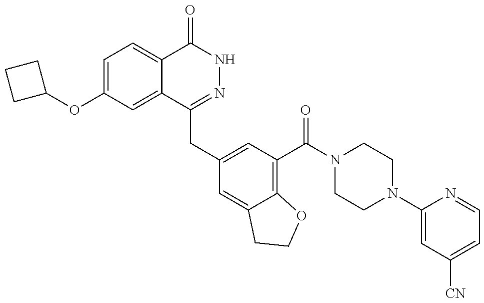 [SMILES: N#Cc1ccnc(N2CCN(C(=O)c3cc(Cc4n[nH]c(=O)c5ccc(OC6CCC6)cc45)cc4c3OCC4)CC2)c1] |
| 176 | 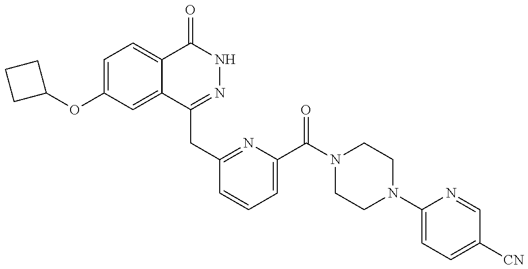 [SMILES: N#Cc1ccc(N2CCN(C(=O)c3cccc(Cc4n[nH]c(=O)c5ccc(OC6CCC6)cc45)n3)CC2)nc1] |
| 177 | 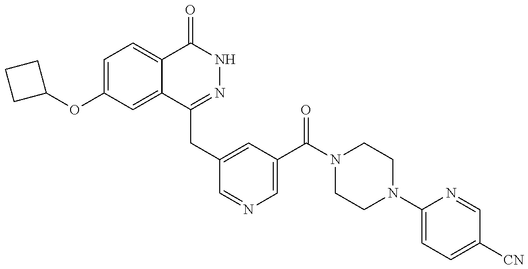 [SMILES: N#Cc1ccc(N2CCN(C(=O)c3cncc(Cc4n[nH]c(=O)c5ccc(OC6CCC6)cc45)c3)CC2)nc1] |
| 178 | 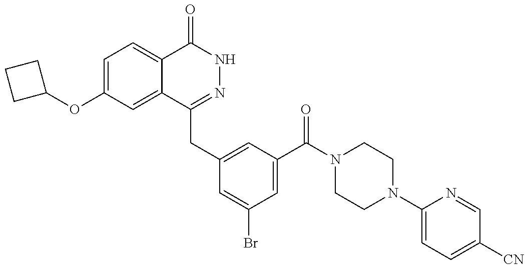 [SMILES: N#Cc1ccc(N2CCN(C(=O)c3cc(Br)cc(Cc4n[nH]c(=O)c5ccc(OC6CCC6)cc45)c3)CC2)nc1] |
| 179 | 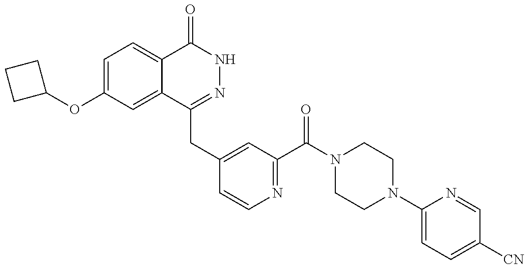 [SMILES: N#Cc1ccc(N2CCN(C(=O)c3cc(Cc4n[nH]c(=O)c5ccc(OC6CCC6)cc45)ccn3)CC2)nc1] |
| 180 | 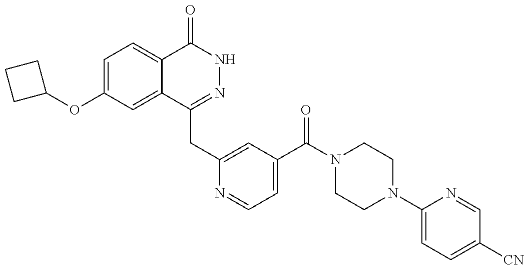 [SMILES: N#Cc1ccc(N2CCN(C(=O)c3ccnc(Cc4n[nH]c(=O)c5ccc(OC6CCC6)cc45)c3)CC2)nc1] |
| 181 | 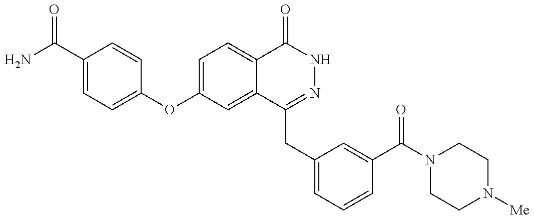 [SMILES: CN1CCN(C(=O)c2cccc(Cc3n[nH]c(=O)c4ccc(Oc5ccc(C(N)=O)cc5)cc34)c2)CC1] |
| 182 | 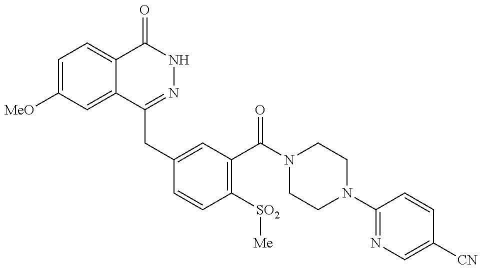 [SMILES: COc1ccc2c(=O)[nH]nc(Cc3ccc(S(C)(=O)=O)c(C(=O)N4CCN(c5ccc(C#N)cn5)CC4)c3)c2c1] |
| 183 | 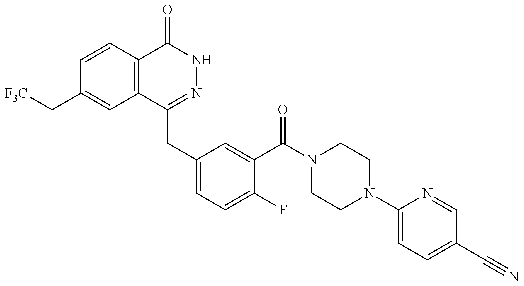 [SMILES: N#Cc1ccc(N2CCN(C(=O)c3cc(Cc4n[nH]c(=O)c5ccc(CC(F)(F)F)cc45)ccc3F)CC2)nc1] |
| 184 | 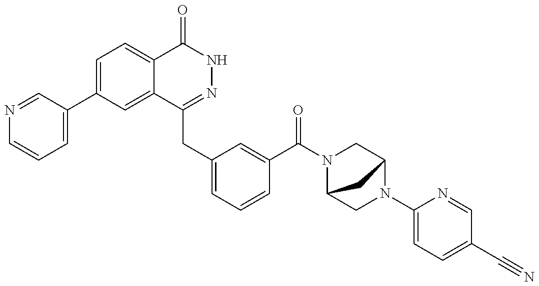 [SMILES: N#Cc1ccc(N2C[C@H]3C[C@@H]2CN3C(=O)c2cccc(Cc3n[nH]c(=O)c4ccc(-c5cccnc5)cc34)c2)nc1] |
| 185 | 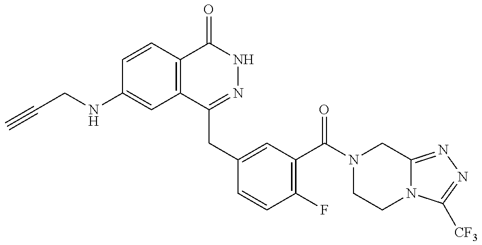 [SMILES: C#CCNc1ccc2c(=O)[nH]nc(Cc3ccc(F)c(C(=O)N4CCn5c(nnc5C(F)(F)F)C4)c3)c2c1] |
| 186 | 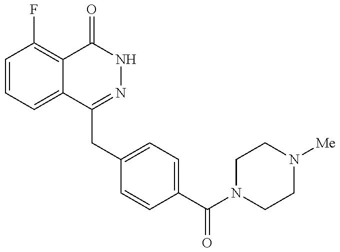 [SMILES: CN1CCN(C(=O)c2ccc(Cc3n[nH]c(=O)c4c(F)cccc34)cc2)CC1] |
| 187 | 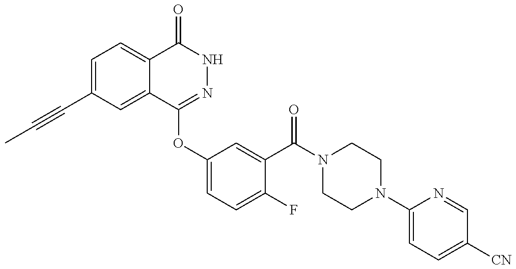 [SMILES: CC#Cc1ccc2c(=O)[nH]nc(Oc3ccc(F)c(C(=O)N4CCN(c5ccc(C#N)cn5)CC4)c3)c2c1] |
| 188 | 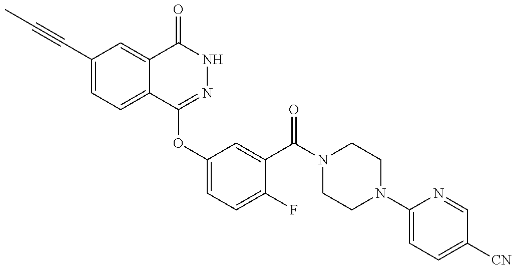 [SMILES: CC#Cc1ccc2c(Oc3ccc(F)c(C(=O)N4CCN(c5ccc(C#N)cn5)CC4)c3)n[nH]c(=O)c2c1] |
| 189 | 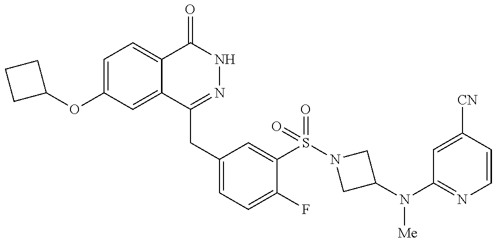 [SMILES: CN(c1cc(C#N)ccn1)C1CN(S(=O)(=O)c2cc(Cc3n[nH]c(=O)c4ccc(OC5CCC5)cc34)ccc2F)C1] |
| 190 | 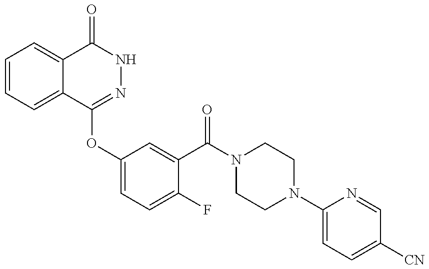 [SMILES: N#Cc1ccc(N2CCN(C(=O)c3cc(Oc4n[nH]c(=O)c5ccccc45)ccc3F)CC2)nc1] |
| 191 | 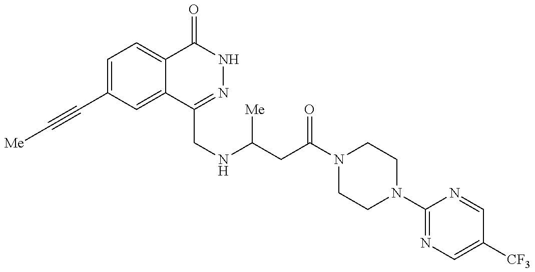 [SMILES: CC#Cc1ccc2c(=O)[nH]nc(CNC(C)CC(=O)N3CCN(c4ncc(C(F)(F)F)cn4)CC3)c2c1] |
| 192 | 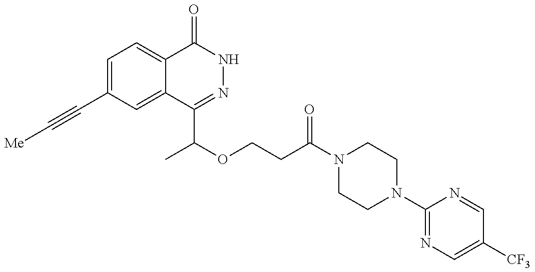 [SMILES: CC#Cc1ccc2c(=O)[nH]nc(C(C)OCCC(=O)N3CCN(c4ncc(C(F)(F)F)cn4)CC3)c2c1] |
| 193 | 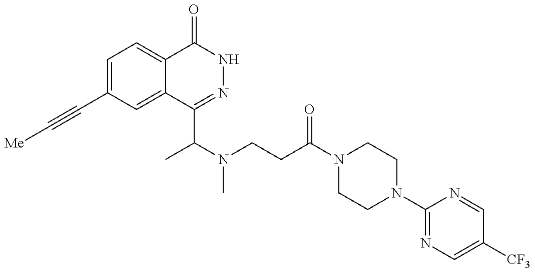 [SMILES: CC#Cc1ccc2c(=O)[nH]nc(C(C)N(C)CCC(=O)N3CCN(c4ncc(C(F)(F)F)cn4)CC3)c2c1] |
| 194 | 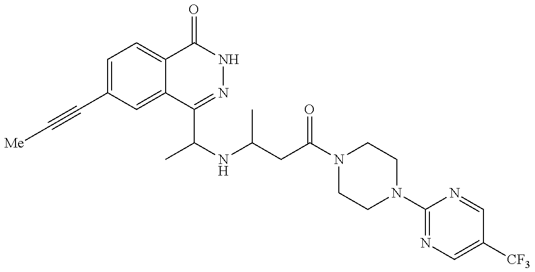 [SMILES: CC#Cc1ccc2c(=O)[nH]nc(C(C)NC(C)CC(=O)N3CCN(c4ncc(C(F)(F)F)cn4)CC3)c2c1] |
| 195 | 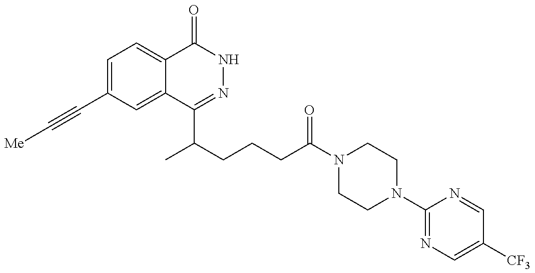 [SMILES: CC#Cc1ccc2c(=O)[nH]nc(C(C)CCCC(=O)N3CCN(c4ncc(C(F)(F)F)cn4)CC3)c2c1] |
| 196 | 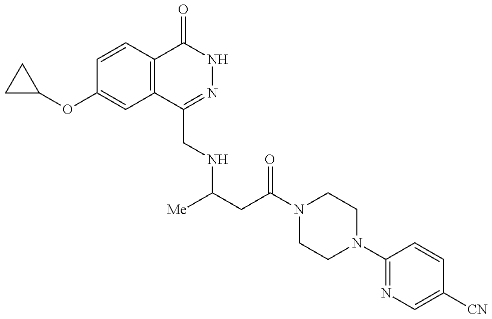 [SMILES: CC(CC(=O)N1CCN(c2ccc(C#N)cn2)CC1)NCc1n[nH]c(=O)c2ccc(OC3CC3)cc12] |
| 197 | 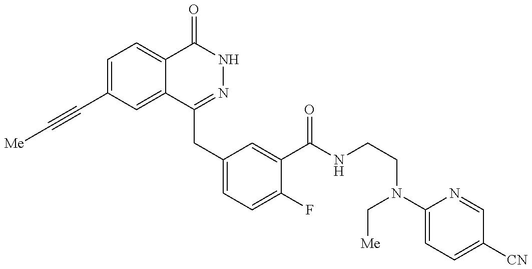 [SMILES: CC#Cc1ccc2c(=O)[nH]nc(Cc3ccc(F)c(C(=O)NCCN(CC)c4ccc(C#N)cn4)c3)c2c1] |
| 198 | 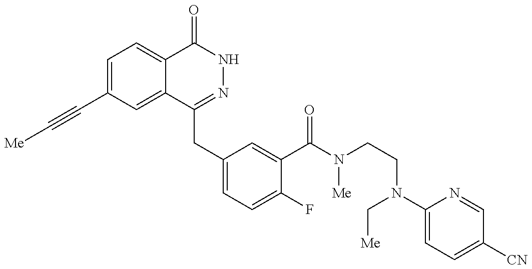 [SMILES: CC#Cc1ccc2c(=O)[nH]nc(Cc3ccc(F)c(C(=O)N(C)CCN(CC)c4ccc(C#N)cn4)c3)c2c1] |
| 199 | 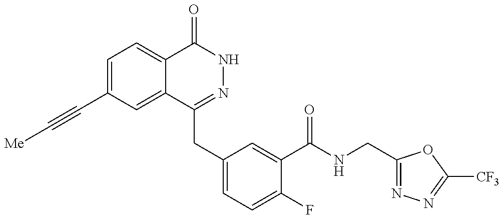 [SMILES: CC#Cc1ccc2c(=O)[nH]nc(Cc3ccc(F)c(C(=O)NCc4nnc(C(F)(F)F)o4)c3)c2c1] |
| 200 | 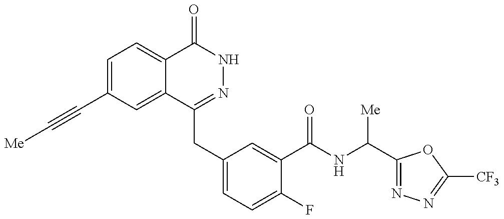 [SMILES: CC#Cc1ccc2c(=O)[nH]nc(Cc3ccc(F)c(C(=O)NC(C)c4nnc(C(F)(F)F)o4)c3)c2c1] |
| 201 | 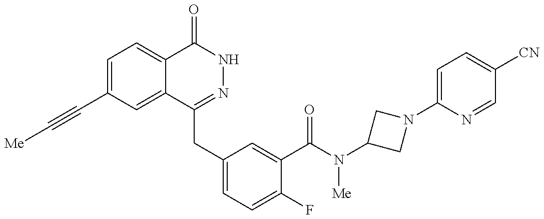 [SMILES: CC#Cc1ccc2c(=O)[nH]nc(Cc3ccc(F)c(C(=O)N(C)C4CN(c5ccc(C#N)cn5)C4)c3)c2c1] |
| 202 | 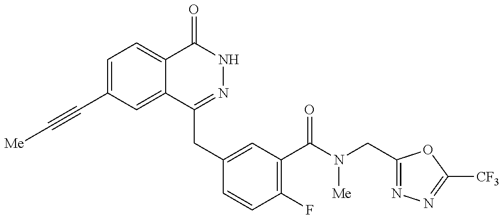 [SMILES: CC#Cc1ccc2c(=O)[nH]nc(Cc3ccc(F)c(C(=O)N(C)Cc4nnc(C(F)(F)F)o4)c3)c2c1] |
| 203 | 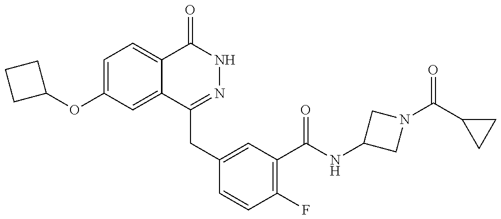 [SMILES: O=C(NC1CN(C(=O)C2CC2)C1)c1cc(Cc2n[nH]c(=O)c3ccc(OC4CCC4)cc23)ccc1F] |
| 204 | 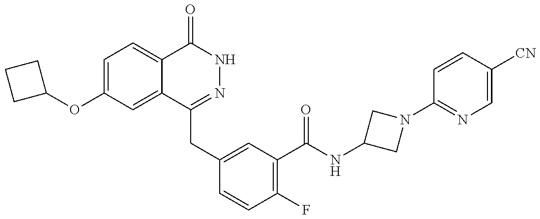 [SMILES: N#Cc1ccc(N2CC(NC(=O)c3cc(Cc4n[nH]c(=O)c5ccc(OC6CCC6)cc45)ccc3F)C2)nc1] |
| 205 | 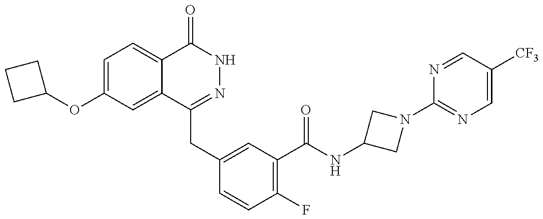 [SMILES: O=C(NC1CN(c2ncc(C(F)(F)F)cn2)C1)c1cc(Cc2n[nH]c(=O)c3ccc(OC4CCC4)cc23)ccc1F] |
| 206 | 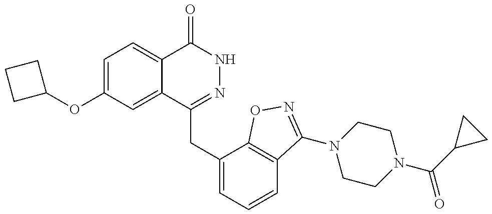 [SMILES: O=C(C1CC1)N1CCN(c2noc3c(Cc4n[nH]c(=O)c5ccc(OC6CCC6)cc45)cccc23)CC1] |
| 207 | 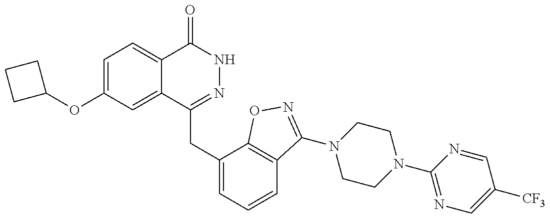 [SMILES: O=c1[nH]nc(Cc2cccc3c(N4CCN(c5ncc(C(F)(F)F)cn5)CC4)noc23)c2cc(OC3CCC3)ccc12] |
| 208 | 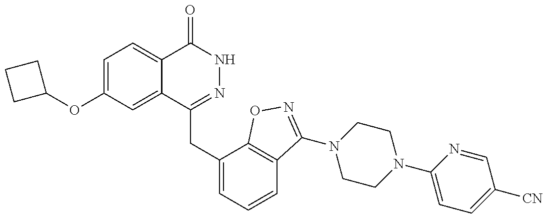 [SMILES: N#Cc1ccc(N2CCN(c3noc4c(Cc5n[nH]c(=O)c6ccc(OC7CCC7)cc56)cccc34)CC2)nc1] |
| 209 | 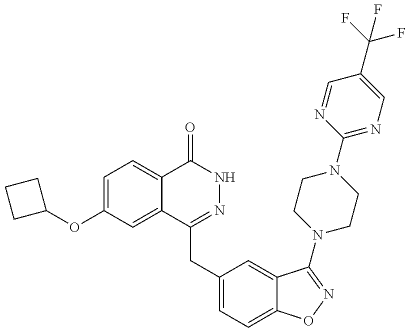 [SMILES: O=c1[nH]nc(Cc2ccc3onc(N4CCN(c5ncc(C(F)(F)F)cn5)CC4)c3c2)c2cc(OC3CCC3)ccc12] |
| 210 | 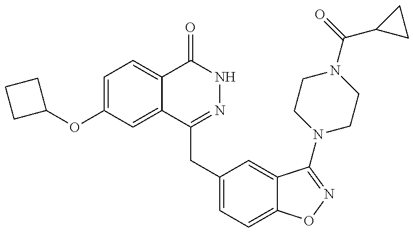 [SMILES: O=C(C1CC1)N1CCN(c2noc3ccc(Cc4n[nH]c(=O)c5ccc(OC6CCC6)cc45)cc23)CC1] |
| 211 | 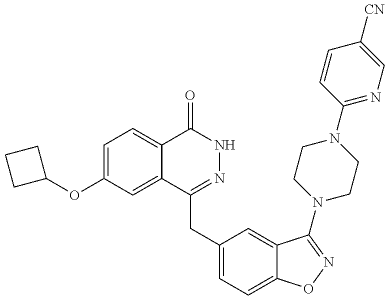 [SMILES: N#Cc1ccc(N2CCN(c3noc4ccc(Cc5n[nH]c(=O)c6ccc(OC7CCC7)cc56)cc34)CC2)nc1] |
| 212 | 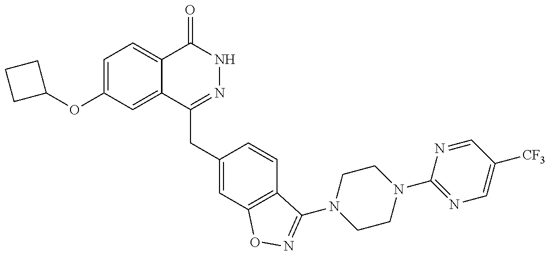 [SMILES: O=c1[nH]nc(Cc2ccc3c(N4CCN(c5ncc(C(F)(F)F)cn5)CC4)noc3c2)c2cc(OC3CCC3)ccc12] |
| 213 | 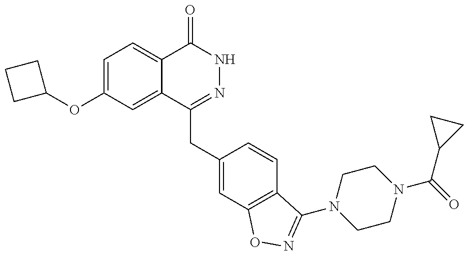 [SMILES: O=C(C1CC1)N1CCN(c2noc3cc(Cc4n[nH]c(=O)c5ccc(OC6CCC6)cc45)ccc23)CC1] |
| 214 | 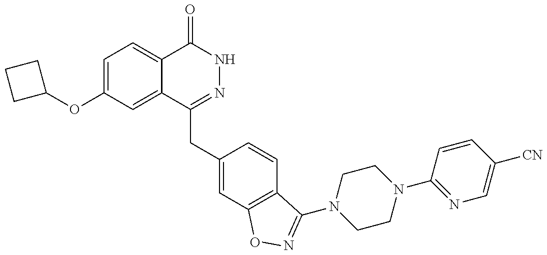 [SMILES: N#Cc1ccc(N2CCN(c3noc4cc(Cc5n[nH]c(=O)c6ccc(OC7CCC7)cc56)ccc34)CC2)nc1] |

### Biological Testing Methods

### Biological Example 1: Protocol for the PARP7 Biochemical Assay

23 μL of 100 nM His-GST-PARP7 in PARP buffer for reaction (PBR) (50 mM HEPES pH 7.5, 100 mM NaCl, 4 mM MgCl₂, 0.2 mM TCEP) was added to each well of XpressBio GSH-coated 384 well plates and incubated for 1 h at RT. The plate was then washed for 3 times in 50 μL PBST (1×PBS, 0.02% Tween-20), followed by one wash in 1×PBS and one wash PBR. Each wash was incubated for 3 min. After washing, varying concentrations of each inhibitor was pre-incubated with 20 μM (2×) 6-a-NAD⁺. The solution was added to the plate, diluted to 1× in PBR, and incubated at 30° C. for 90 min. Following the enzymatic reaction, the plate was washed 3 times in PBST, once in PBS for 3 min each wash. The wells were then subjected to 25 μL of click reaction buffer consisting of 100 μM TBTA, 1 mM CuSO₄, 100 μM Biotin-N₃, 2 mM TCEP made up in 1×PBS. The wells were incubated in click reaction buffer for 30 min at 30° C. The click buffer was removed, and the plate was washed 3 times with PBST and once with PBS as before. The plate was then blocked with 5% BSA in PBST. The plate was then washed 3 times with PBST and once with PBS. To develop, the plate was incubated with 25 μL of 0.05 ng/μL Step-HRP with 0.02% BSA in PBS. The plate was washed 3 times with PBS before developing with QuantaRed per manufacturer directions. Fluorescence (Excitation 550 nm. Emission 620 nm) was immediately read using a Paradigm Plate Reader.

### Biological Example 2: Protocol for the PARP1 and PARP2 Biochemical Assays

N-Terminal His-tagged PARPs (PARP1, PARP2) and SRPK2 were expressed and purified as previously described (Carter-O'Connell et al., J. Am. Chem. Soc. 2014, 136, 14, 5201-5204.) PARP1 was purified to greater than 90% and SRPK2 to 70% or greater by an in-gel standard curve of Bovine Serum Albumin (Bio-Rad). PARP1 and PARP2 plate assays were performed as described in (Kirby et al. STAR Protocols, 2021, 2(1), 100344.) 50 μL of 1 μM SRPK2 in PARP Buffer for reaction (PBR) (50 mM HEPES pH 7.5, 100 mM NaCl, 4 mM MgCl₂, 0.2 mM TCEP) was added to wells of a Nickel-NTA coated 96-well plate (Thermo Scientific) and incubated at RT for 1 h. The solution was removed, and the plate was washed 3 times with PBST (1×PBS, 0.1% Tween-20), once with 1×PBS, and once with PBR. Each wash was incubated for 3 min. After washing, varying concentrations of each inhibitor was pre-incubated with 20 μM (2×) 6-a-NAD⁺. 25 μL of the 2×6-a-NAD⁺/inhibitor mixture was added to the plate with 25 μL of 20 nM of PARP1 or PARP2 in PBR with 0.1 mg/mL Activated DNA (Sigma). PARP final concentration 10 nM. The reaction mixture was incubated at 30° C. for 1 h. The plate was then washed 3 time with PBST and once with PBS. The wells were subjected to a 50 μL click reaction consisting of 100 μM TBTA, 1 mM CuSO₄. 100 μM Biotin-N₃, 2 mM TCEP made up in 1×PBS at RT for 30 min. The plate was then washed 3 times with PBST before blocking in 100 μL of 1% Milk (Carnation) in PBST at RT for 30 min. The plate was again washed 3 times with PBST and once with PBS before incubation with Strep-HRP (0.05 ng/μL Strep-HRP (Fisher Scientific), 300 ng/μL BSA, 1×PBS) for 30 min at RT. The plate was washed 3 time in PBST and once with PBS before development with QuantaRed (Pierce) per manufacturer directions. The plate was immediately read for fluorescence (Excitation 550 nm, Emission 620 nm) on Spectra Max i3 (Molecular Devices) following development. Three parameter nonlinear regression was performed with GraphPad Prism 9 to obtain ICs, values.

### Biological Example 3: Protocol for the Cell Proliferation Assays Using NCI-H1373 (Human Lung Adenocarcinoma), HARA (Human Lung Squamous Cell Carcinoma) and CT26 (Mouse Colon Adenocarcinoma) Cell Lines

An assay to measure inhibition of NCI-H1373, HARA or CT26 cell growth was performed using CellTiter-Glo (CTG) One Solution Assay reagent (for detection of ATP by luminescence; Promega, G8461) and an EnVision microplate reader (Perkin Elmer, 2104-0010) in a 384-well format. On the day prior to treatment. NCI-H1373, HARA or CT26 cells were dissociated using trypsin-EDTA 0.25% and seeded in white 384-well plates at a density of 500 cells per well in medium (RPMI 1640 with L-glutamine+10% FBS). Outermost wells were filled with only medium to mitigate edge effects. On the day of treatment, one row of wells in one plate was treated with CTG reagent. The plate was then covered with a clear plate seal and a white adhesive backing and read on the EnVision plate reader 30 minutes after the addition of CTG reagent in order to provide a Day 0 measurement of cell viability. Serial dilutions of compounds to be tested were prepared in DMSO, then all further diluted by the same factor in medium to minimize potential precipitation. Compounds diluted in medium were then transferred to the 384-well NCI-H1373. HARA or CT26 plates (0.5% DMSO final). Plates were cultured for 6 days. On Day 6, viability was measured using CTG reagent as described above. Growth curves and GI₅₀ values were calculated using Excel (Microsoft Office Professional Plus 2013) with XLFit add-in (ID Business Solutions Limited, version 5.5.0.5). The average Day 0 reading was subtracted from all Day 6 readings, and cell viability for each inhibitor at each concentration was calculated as a percentage of average vehicle-only reading. Fit Model (205) Dose Response One Site (a 4 parameter logistic model) was applied (Fit=(A+((B=A)/(1+((C/x)\hat{ }D)))); Inv=(C/((((B−A)/(y−A))−1)\hat{ }(1/D))); Res=(y-fit)). This model was used to calculate absolute GI₅₀, values (compound concentration at which cell viability is 50% of untreated control).

### Results

Results of certain compounds are shown in Table 3. Table legend:

IC₅₀ Range Values for PARP7, PARP1 and PARP2 biochemical assays: **** is [IC₅₀]<50 nM; *** is 50 nM≤[IC₅₀]<100 nM; ** is 100 nM≤[IC₅₀]<500 nM; * is 500 nM≤[IC₅₀]<2000 nM; − is 2000 nM≤[IC₅₀] where [IC₅₀] is the IC₅₀ concentration value in nanomolar units.

GI₅₀ Range Values for NCI-H1373. HARA and CT26 cell proliferation assays: +++ is [GI₅₀]<100 nM; ++ is 100 nM≤[GI₅₀]<1000 nM: + is 1000 nM≤[GI₅₀]<5000 nM; \hat{ } is 5000 nM≤[GI₅₀] where [GI₅₀] is the GI₅₀ concentration value in nanomolar units.

TABLE 3

| | PARP7 | PARP1 | PARP2 | NCI-H1373 | HARA | CT26 |
|---|---|---|---|---|---|---|
| Example | IC₅₀ | IC₅₀ | IC₅₀ | GI₅₀ | GI₅₀ | GI₅₀ |
| 1 | **** | | **** | | | |
| 2 | **** | | | +++ | | |
| 3 | **** | | **** | +++ | + | |
| 4 | **** | | | +++ | + | ++ |
| 5 | **** | | | ++ | | |
| 6 | **** | | | ++ | | |
| 7 | **** | | ** | +++ | | |
| 8 | **** | ** | ** | +++ | + | +++ |
| 9 | **** | | | ++ | | |
| 10 | **** | | ** | +++ | + | \hat{ } |
| 11 | **** | | | ++ | | |
| 12 | **** | | | ++ | | |
| 13 | **** | | ** | ++ | | |
| 14 | **** | | * | +++ | + | |
| 15 | **** | | | + | | |
| 16 | **** | | **** | + | | |
| 17 | **** | | | + | | |
| 18 | **** | | ** | | | |
| 19 | **** | | | ++ | | |
| 20 | **** | | **** | ++ | | |
| 21 | **** | | | ++ | | |
| 22 | **** | | | + | | |
| 23 | **** | | * | \hat{ } | | |
| 24 | **** | | ** | | | |
| 25 | **** | | ** | | | |
| 26 | **** | | ** | | | |
| 27 | **** | | *** | | | |
| 28 | **** | | ** | | | |
| 29 | **** | | | ++ | | |
| 30 | **** | | ** | ++ | | |
| 31 | **** | | − | ++ | | |
| 32 | **** | | | | | |
| 33 | **** | | | + | | |
| 34 | **** | | * | ++ | | |
| 35 | *** | | | | | |
| 36 | *** | | **** | ++ | | |
| 37 | ** | | * | | | |
| 38 | **** | | | | | |
| 39 | *** | | | | | |
| 40 | **** | | ** | | | |
| 41 | ** | | ** | | | |
| 42 | * | | | | | |
| 43 | **** | | ** | | | |
| 44 | **** | | *** | | | |
| 45 | **** | | ** | | | |
| 46 | **** | | ** | | | |
| 47 | ** | | * | | | |
| 48 | ** | | − | | | |
| 49 | ** | | ** | | | |
| 51 | **** | | | +++ | \hat{ } | + |
| 52 | | | | | | |
| 53 | ** | | | \hat{ } | \hat{ } | \hat{ } |
| 54 | * | | | | | |
| 55 | ** | | | | | |
| 56 | **** | | | ++ | \hat{ } | |
| 57 | **** | | | | | |
| 58 | **** | | | \hat{ } | | |
| 59 | ** | | | | | |
| 60 | **** | | | + | | |
| 61 | **** | | **** | | | |
| 62 | **** | | * | | | |
| 63 | **** | | | \hat{ } | | |
| 64 | **** | | | | | |
| 65 | **** | *** | *** | ++ | | |
| 66 | **** | | | + | | |
| 67 | *** | | | + | | |
| 68 | * | | | | | |
| 69 | − | | | \hat{ } | | |
| 70 | − | | | \hat{ } | | |
| 71 | **** | | | + | | |
| 72 | **** | | ** | | | |
| 73 | **** | | | | | |
| 74 | **** | | *** | ++ | | |
| 75 | − | | | | | |
| 76 | − | | | | | |
| 77 | **** | | | | | |
| 78 | − | | | | | |
| 79 | ** | | | | | |
| 80 | **** | | | ++ | + | |
| 81 | **** | | | +++ | ++ | +++ |
| 82 | **** | **** | **** | +++ | ++ | +++ |
| 83 | **** | | | ++ | | |
| 84 | **** | ** | ** | +++ | + | \hat{ } |
| 85 | ** | | *** | +++ | | |
| 86 | **** | *** | * | +++ | + | \hat{ } |
| 87 | **** | **** | **** | +++ | ++ | ++ |
| 88 | **** | | | ++ | | |
| 89 | *** | | | | | |
| 90 | **** | | | | | |
| 91 | **** | | | + | \hat{ } | \hat{ } |
| 92 | *** | | | \hat{ } | \hat{ } | |
| 93 | **** | | | +++ | + | + |
| 94 | **** | | | +++ | + | + |
| 95 | **** | | | | | |
| 96 | **** | | | | | |
| 97 | **** | | | | | |
| 98 | **** | | | ++ | \hat{ } | |
| 99 | **** | | | | | |
| 100 | **** | | | | | |
| 101 | **** | | | \hat{ } | \hat{ } | |
| 102 | *** | | | | | |
| 103 | *** | | | | | |
| 104 | *** | | | | | |
| 105 | *** | | | \hat{ } | | |
| 106 | ** | | | | | |
| 107 | ** | | | | | |
| 108 | ** | | | | | |
| 109 | ** | | | | | |
| 110 | ** | | | | | |
| 111 | ** | | | | | |
| 112 | ** | | | | | |
| 113 | ** | | | | | |
| 114 | ** | | | | | |
| 115 | ** | | | | | |
| 116 | ** | | | | | |
| 117 | * | | | | | |
| 118 | * | | | | | |
| 119 | * | | | | | |
| 120 | * | | | | | |
| 121 | * | | | | | |
| 122 | * | | | | | |
| 123 | * | | | | | |
| 124 | − | | | | | |
| 125 | **** | | | ++ | \hat{ } | |
| 126 | **** | | | ++ | \hat{ } | |
| 127 | **** | | | + | | |
| 128 | **** | | | ++ | + | + |
| 129 | **** | | | + | + | |
| 130 | **** | | | ++ | \hat{ } | |
| 131 | **** | | | ++ | + | |
| 132 | **** | − | − | +++ | + | |
| 133 | **** | | | ++ | \hat{ } | |
| 134 | **** | | | +++ | ++ | |
| 135 | *** | | | ++ | \hat{ } | \hat{ } |
| 136 | *** | | | ++ | \hat{ } | |
| 137 | **** | − | ** | +++ | + | |
| 138 | **** | | | ++ | + | \hat{ } |
| 139 | **** | | | ++ | + | \hat{ } |
| 140 | **** | | | + | | |
| 141 | *** | | | | | |
| 142 | ** | | | | | |
| 143 | *** | | | | | |
| 144 | **** | | | | | |
| 145 | **** | | | + | | |
| 146 | **** | | | \hat{ } | | |
| 147 | − | | | | | |
| 148 | * | | | \hat{ } | \hat{ } | \hat{ } |
| 149 | * | | | \hat{ } | \hat{ } | \hat{ } |
| 150 | **** | | | ++ | + | + |
| 151 | | | | | | |
| 152 | | | | | | |
| 154 | **** | | | +++ | + | + |
| 155 | **** | | | +++ | + | |
| 156 | **** | | | ++ | + | \hat{ } |
| 157 | **** | | | +++ | + | + |
| 158 | **** | | | + | \hat{ } | + |
| 159 | **** | | | ++ | \hat{ } | \hat{ } |
| 160 | **** | | | +++ | + | |
| 161 | **** | | | | | |
| 162 | **** | | | +++ | ++ | |
| 163 | **** | | | | | |
| 164 | **** | | | | | |
| 165 | **** | | | | | |
| 166 | **** | | | | | |
| 167 | **** | | | | | |
| 168 | ** | | | \hat{ } | \hat{ } | \hat{ } |
| 169 | **** | | | +++ | | |
| 170 | **** | | | | | |
| 171 | *** | | | | | |
| 172 | − | | | | | |
| 173 | * | | | | | |
| 174 | **** | | | | | |
| 175 | *** | | | | | |
| 176 | **** | *** | ** | ++ | \hat{ } | + |
| 177 | **** | *** | ** | +++ | \hat{ } | \hat{ } |
| 178 | **** | | | ++ | + | \hat{ } |
| 179 | **** | | | +++ | \hat{ } | \hat{ } |
| 180 | **** | | | +++ | \hat{ } | + |
| 181 | * | | | | | |
| 182 | **** | | | \hat{ } | | |
| 183 | **** | | | | | |
| 184 | ** | | | | | |
| 185 | ** | | | \hat{ } | | |
| 186 | − | | | | | |
| 187 | ** | | | + | | |
| 188 | − | | | | | |
| 189 | * | | | | | |
| 190 | ** | | | + | | |
| 191 | **** | − | − | ++ | + | \hat{ } |
| 192 | **** | | | ++ | | |
| 193 | **** | | − | ++ | | |
| 194 | *** | | | ++ | | |
| 195 | **** | | | | | |
| 196 | **** | | | + | + | |
| 197 | **** | | − | | | |
| 198 | **** | | − | | | |
| 199 | − | | | + | | |
| 200 | − | | | | | |
| 201 | * | | | + | | |
| 202 | ** | | | | | |
| 203 | ** | | | | | |
| 204 | − | | | | | |
| 205 | − | | | | | |
| 206 | ** | | | | | |
| 207 | − | | | | | |
| 208 | ** | | | + | + | |
| 209 | − | | | | | |
| 210 | * | | | | | |
| 211 | ** | | | + | + | |
| 212 | − | | | | | |
| 213 | − | | | | | |
| 214 | − | | | | | |

### Biological Example 4: Structurally Distinct PARP7 Inhibitors Reveal New Insights into the Regulation of Cytosolic Nucleic Acid-Sensing by PARP7

### Summary

The mono-ADP-ribosyltransferase PARP7 is an aryl hydrocarbon receptor (AHR)-stimulated gene that has emerged as a negative regulator of cytosolic nucleic acid (NA)-sensors of the innate immune system. Herein, we apply a rational design strategy for parlaying a pan-PARP inhibitor into a selective inhibitor of PARP7 (Example 10). Example 10 potently inhibits PARP7-mediated MARylation in vitro and in cells, and displays PARP family-wide selectivity. Consistent with recent studies using structurally distinct PARP7 inhibitor RBN-2397, co-treatment of mouse embryonic fibroblasts (MEFs) with Example 10 and an AHR agonist synergistically induced the expression of an AHR-target gene. Likewise, co-treatment of MEFs with Example 10 and cytoplasmic NA-sensor ligands synergistically induced the expression of the type 1 interferon. IFN-β. In mouse colon carcinoma (CT-26) cells, Example 10 treatment alone induced IFN-β signaling in a STING-dependent manner. Both Example 10 and RBN-2397 increased endogenous PARP7 protein levels in the nucleus of CT-26 cells, demonstrating that the catalytic activity of PARP7 regulates its protein levels in cells. Curiously, saturating doses of Example 10 and RBN-2397 achieved different maximum levels of PARP7 protein, which correlated with the magnitude of type I interferon gene expression.

### Introduction

Cytosolic nucleic acid (NA)-sensing serves as the first line of defense against pathogens. When viruses and bacteria enter cells, they release their NAs into the cytoplasm, which are promptly detected by cytosolic NA sensors. DNA derived from viruses and bacteria is recognized by cytosolic NA sensors, namely cyclic cGMP-AMP (cGAMP) synthase (cGAS). Binding of pathogen-derived DNA to cGAS stimulates the production of cGAMP, which binds to and activates stimulator of interferon genes protein (STING). RNA derived from viruses is recognized by a different set of cytosolic NA sensors, the most well-studied being retinoic acid-inducible gene 1 (RIG-1). While the activation mechanisms of STING and RIG-1 are distinct, they both converge on common signal transducers, such as TANK binding kinase 1 (TBK1) and its target interferon regulatory factor 3 (IRF3), the activation of which leads to type I interferon (IFN-I) production. IFN-I functions as both an autocrine and paracrine factor to activate the Janus kinase (JAK)-signal transducer and activator of transcription (STAT) pathway and subsequently induce interferon stimulated genes or (ISGs), which enhance the innate response and orchestrate an adaptive immune response (e.g., activation of cytotoxic T cells to kill infected cells). While involved in host defense against pathogens, overactivation of cytosolic NA-sensing can lead to autoimmune and inflammatory disorders. Thus, repressors of cytosolic NA-sensing are potentially useful for attenuating innate immunity.

PARP7 (also known as TiPARP) has emerged as a potential repressor of cytosolic NA-sensing. PARP7 is a member of a family of 17 enzymes in humans called PARPs. PARP7, like most other PARP family members, catalyzes the transfer of ADP-ribose (ADPr) from nicotinamide adenine dinucleotide (NAD⁺) to amino acids on protein targets, a post-translational modification (PTM) known as mono-ADP-ribosylation (MARylation). PARP7 was initially identified as a gene that is strongly upregulated by synthetic agonists (e.g., the carcinogen 2,3,7,8-tetrachlorodibenzo-p-dioxin or TCDD) of the aryl hydrocarbon receptor (AHR). Subsequent studies demonstrated that PARP7 is a negative regulator of AHR-ligand induced transcription. The repressor function of PARP7 is dependent on its catalytic activity, and it is postulated that this is due to MARylation of AHR, although the consequences of AHR MARylation on its function are not understood.

Recently, PARP7 was identified as an AHR regulated gene that represses IFN-I signaling in response to cytosolic NA-sensor ligands and several types of RNA viruses. In mouse embryonic fibroblasts (MEFs), knockout of PARP7 in the presence of cytosolic NA-sensor ligands (i.e. the synthetic RIG-I ligand 3pRNA and the STING agonist cGAMP) synergistically induced IFN-β expression. Expression of wild-type (WT), but not a catalytically inactive PARP7 mutant in PARP7 knockout MEFs rescued these effects, suggesting that the catalytic activity of PARP7 is potentially important for repressing cytosolic NA sensing and downstream IFN-β signaling.

Beyond host defense against pathogens, cytosolic NA-sensing—in particular the cGAS/STING pathway—is involved in preventing an inappropriate immune response to cytosolic self-DNA. Cancer cells contain cytosolic self-DNA (e.g., due to defects in DNA repair) and must repress cGAS/STING to evade detection and elimination by adaptive immune responses. Therefore repressors of cGAS/STING, like PARP7, are potential therapeutic targets in the burgeoning area of immune-oncology. Indeed, a pyridazinone-based PARP7 inhibitor (RBN-2397) treatment induced STING-dependent, IFN-I signaling in certain cancer cells, resulting in immune-cell dependent killing in vivo. This study not only validates the role of PARP7 as a repressor of cGAS/STING signaling, but also demonstrates the therapeutic potential of PARP7 inhibitors as immunomodulatory agents.

Intriguingly, stable knockout of PARP7 in mouse colorectal cancer cells (CT-26 cells) did not affect tumor growth in vivo and only weakly activated IFN-I signaling in vitro. Moreover, a CRISPR/Cas9 screen identified PARP7 itself as a hit for conferring RBN-2397 resistance in NIH-1373 cells. Together, these results suggest that the immunomodulatory effects of RBN-2397 are not simply due to catalytic inhibition, but may involve other mechanisms dependent on PARP7.

Here, we used rational drug design to convert a PARP1/2/7 inhibitor into a selective PARP7 inhibitor (Example 10). Example 10 achieves selectivity for PARP7 over PARP1/2 by exploiting a hydrophobic sub-pocket adjacent to the NAD⁺ binding site found in PARP7 but not PARP1/2. Cell-based studies using Example 10 validate the requirement of PARP7's catalytic activity for its repressor functions in both AHR and IFN-I signaling. Example 10, as well as RBN-2397 increase PARP7 protein levels in the nucleus of CT-26 cells, suggesting that PARP7 stability is regulated by its activity. Yet despite being equipotent against PARP7 catalytic activity in vitro. RBN-2397 leads to a two-fold higher increase in PARP7 protein levels at saturating doses. Intriguingly, this difference correlated with a two-fold higher induction of an IFN-I-responsive luciferase reporter, potentially suggesting that, in addition to serving as derepressor of IFN-I-mediated transcription. PARP7 inhibitors may induce cGAS/STING signaling via a PARP7-inhibitor complex.

Parlaying a PARP1/2/7 Inhibitor into a Selective PARP7 Inhibitor

MARylating PARPs—like PARP7—are the largest sub-group of the PARP family, yet are the least understood. This is because unlike PARylating PARPs, namely PARP1 and PARP2, there is a dearth of highly selective MARylating PARP inhibitors. An obstacle toward the development of a selective inhibitor of PARP7, or any PARP, is the high structural conservation among PARP catalytic domains; while the sequence homology is only ˜50%, their structures are highly conserved, especially in the NAD⁺ binding site, which is the major binding site for the majority of PARP inhibitors. To address this challenge, we identified previously a unique, hydrophobic cavity abutting the nicotinamide sub-pocket of the NAD⁺ binding site in MARylating that can be exploited for the development of selective inhibitors of MARylating PARPs. Crystal structure analysis of one of our MARylating PARP inhibitors. ITK6, bound to PARP14 (PDB: 6FZM) shows that Leu1782 (human PARP14) acts as a “gatekeeper” (a term we borrow from the protein kinase field), allowing hydrophobic substituents emanating from the inhibitor scaffold to access the hydrophobic cavity. A hydrophobic amino acid at the gatekeeper position is conserved across the MARylating PARP sub-group, and indeed a growing number of MARylating PARPs exploit the hydrophobic cavity. PARylating PARPs have a conserved glutamate at the gatekeeper position, and analysis of multiple structures of PARP1 and PARP2 shows that this glutamate occludes access to the hydrophobic cavity, consistent with previous studies showing that MARylating PARP inhibitors containing substituents that access the hydrophobic cavity show selectivity over PARylating PARPs.

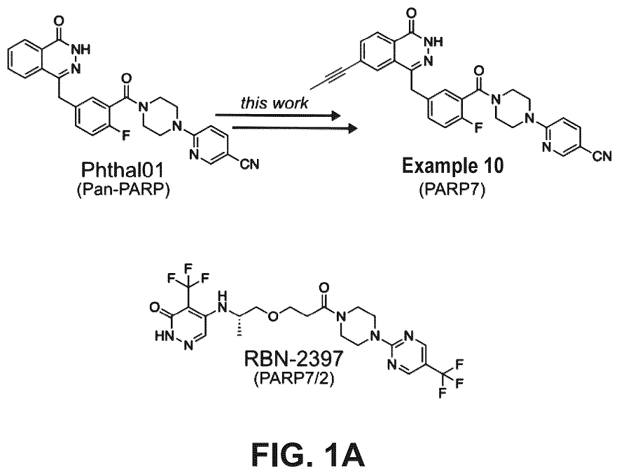

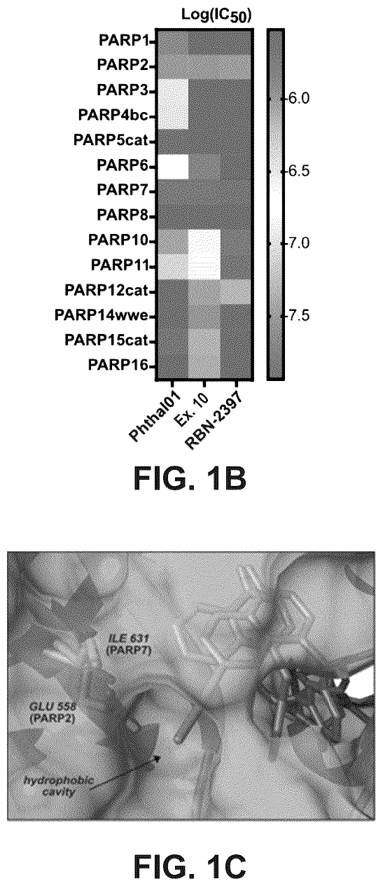

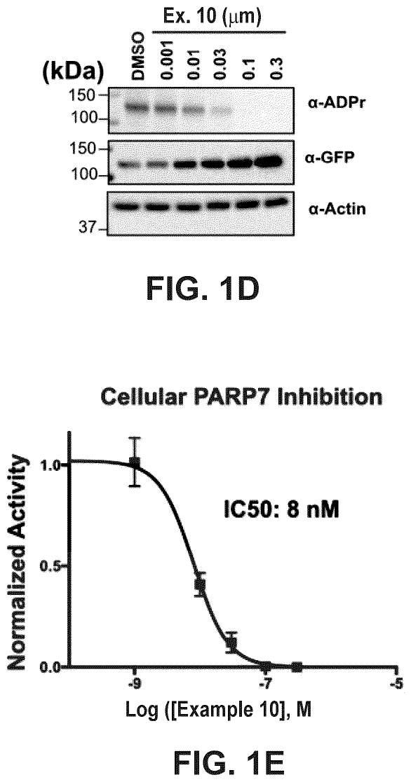

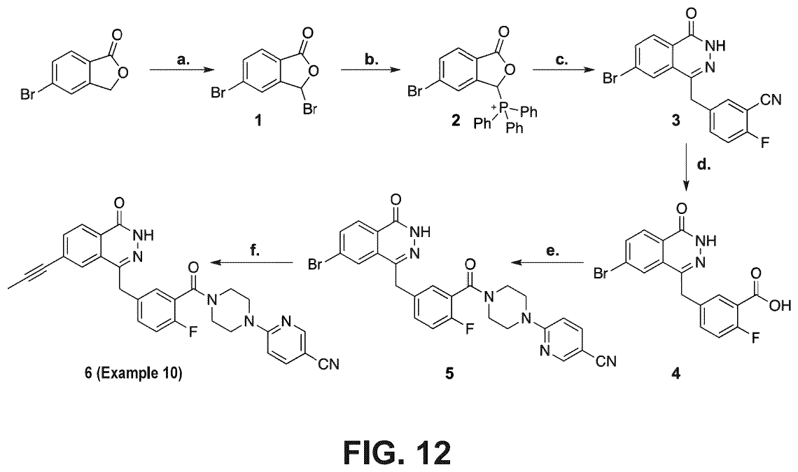

A step in our rational design strategy is the identification of a NAD⁺-competitive inhibitor scaffold that contains a position that can be easily derivatized with a substituent that will interact favorably with the unique hydrophobic cavity in MARylating PARPs. In recent work, we found that the NAD⁺-competitive inhibitor Phthal01 (FIG. 1A), which is based on a phthalazinone scaffold, is a potent, but non-specific inhibitor of PARP7. Using our PARP inhibitor screening platform, PARP activity screening and inhibitor testing assay (PASTA), we found that Pthal01 exhibited double-digit nanomolar potency against three PARPs: PARP1, 2, and PARP7, but was at least 12-fold more potent against PARP7 compared to all other PARP family members (FIG. 1B). To parlay Phthal01 into a more selective inhibitor of PARP7, we designed and synthesized a Phthal01 analogue (Example 10) that contains a propynyl at the C-6 position of the phthalazinone scaffold (FIG. 1A and FIG. 12). We hypothesized that the C-6 propynyl would occupy the hydrophobic cavity in PARP7, but would clash with the glutamate gatekeeper in PARylating PARPs. We chose a propynyl because our previous studies suggested that this was a privileged substituent for generating selective MARylating PARP inhibitors.

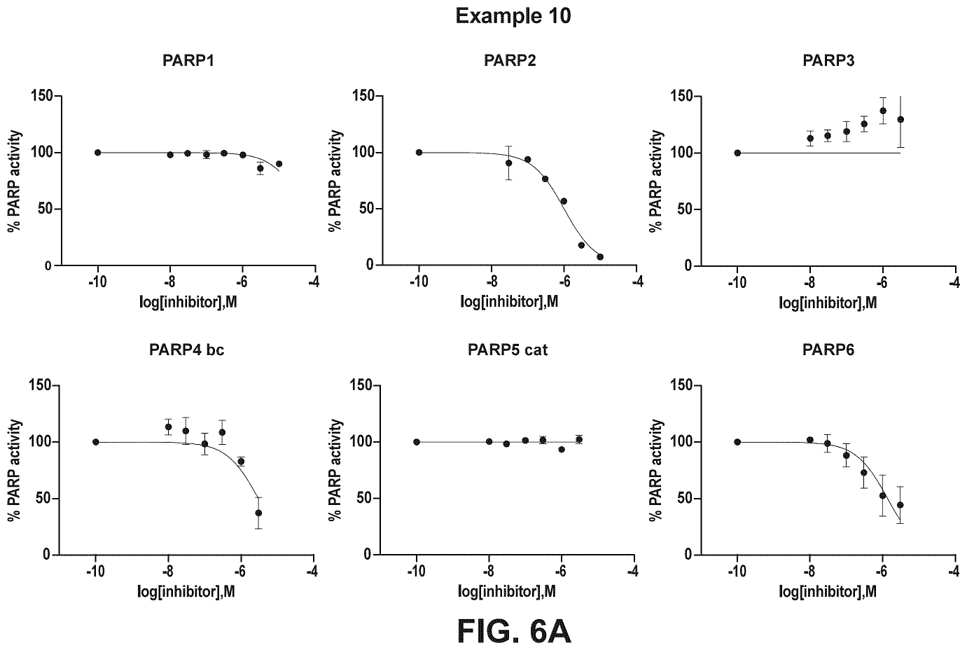

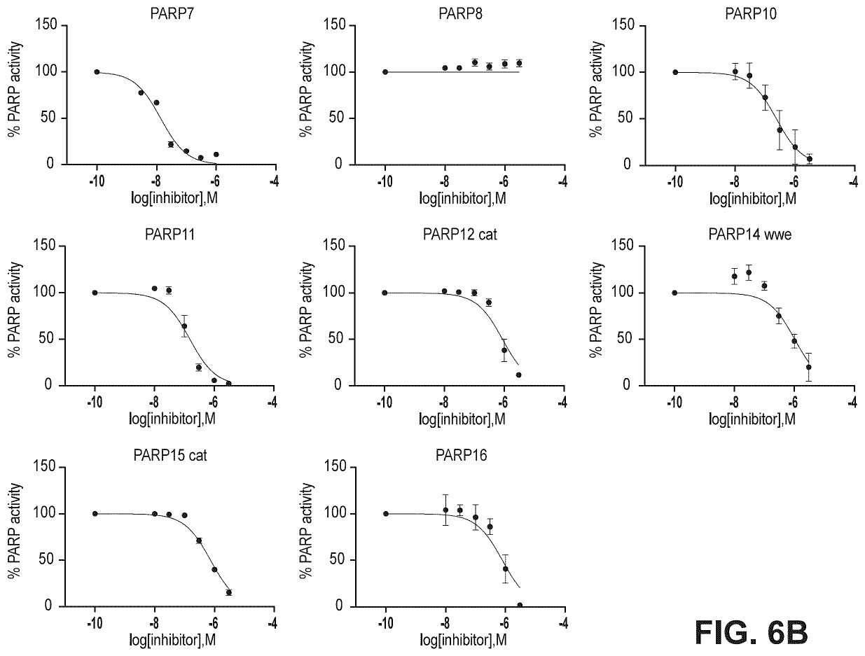

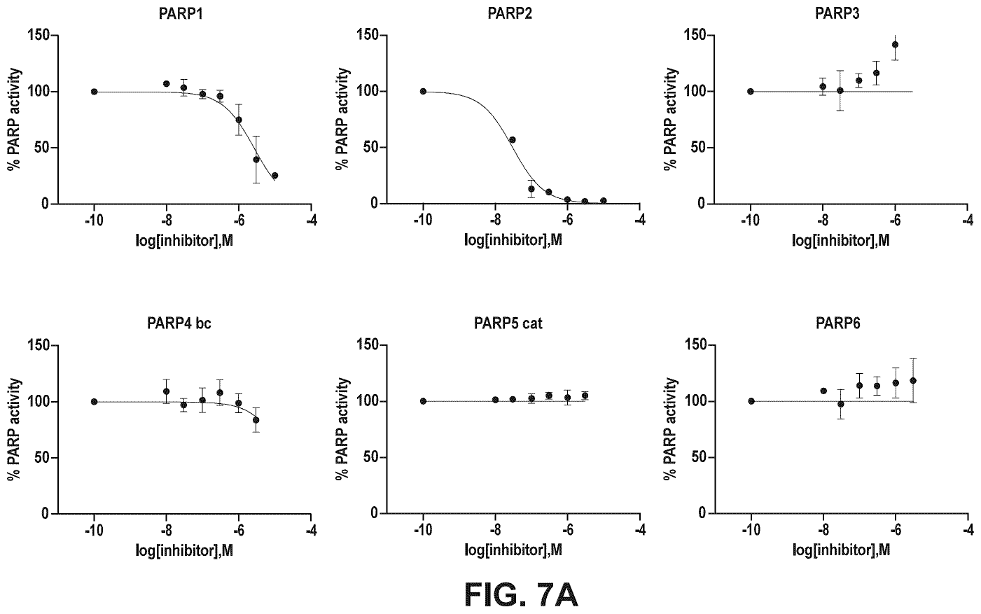

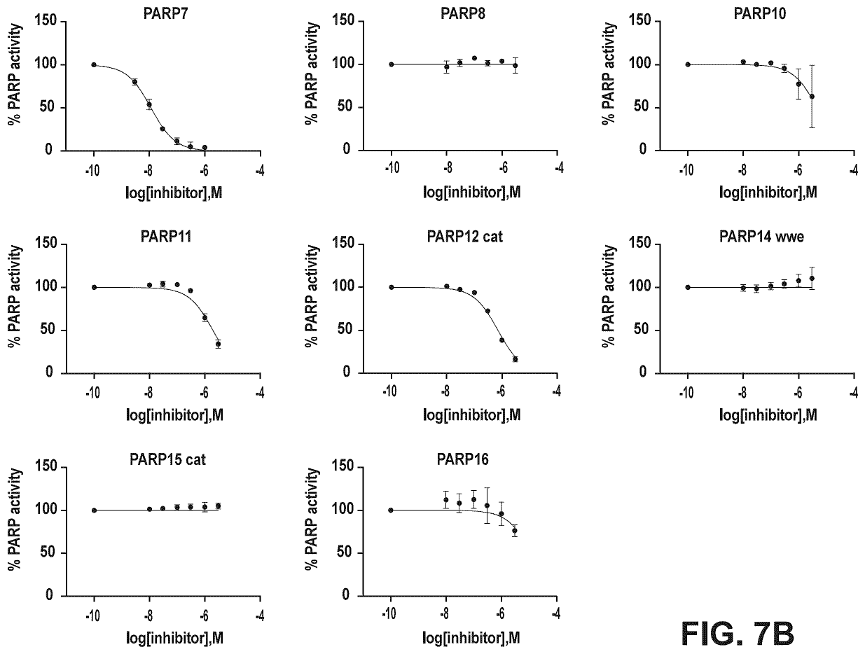

We used PASTA to profile the selectivity of Example 10, as well as the recently described PARP7 inhibitor RBN-20397 (FIG. 1A), across the PARP family. Similar to Phthal01, we found that Example 10 potently inhibits PARP7 with an IC₅₀=13.7 nM (FIG. 1B. FIG. 6, and Table 4); however unlike Pthal01, Example 10 displays ˜75-fold selectivity for PARP7 over PARP2 and it does not inhibit PARP1 up to 3 μM. Consistent with the previous study that first described RBN-2397, we found that RBN-2397 potently inhibits PARP2 (IC₅₀=30.3 nM), but does not inhibit most other PARP family members up to 3 μM. However, we did identify two discrepancies: using PASTA, we found that RBN-2397 weakly inhibits PARP1 (IC₅₀=2639 nM) and PARP12 (IC₅₀=716 nM) whereas the previous study found that RBN-2397 was more potent (i.e. double digit nanomolar inhibition) against these PARPs. While the PARP inhibitor screening assay used in the previous study is similar to PASTA, there are a few differences that could account for this variance. Our in vitro IC₅₀ values for RBN-2397 against PARP1 and PARP12 are congruent with previously obtained cellular IC₅₀ values for RBN-2397 against PARP1 and PARP12, suggesting PASTA accurately predicates the cellular efficacy of PARP inhibitors. Example 10 is >50-fold selective for PARP7 over most PARP family members beyond PARP1 and PARP2; two exceptions are PARP10 and PARP11, however. Example 10 still exhibits ˜10-fold selectivity for PARP7 over these MARylating PARPs. The excellent family-wide selectivity of Example 10 for PARP7 over other PARP family members makes it a useful chemical tool for probing PARP7-specific functions in physiological and pathophysiological contexts.

TABLE 4

| | Phthal01 | | Example 10 | | RBN - 2397 | |
|---|---|---|---|---|---|---|
| | IC₅₀ | 95% Confidence | IC₅₀ | 95% Confidence | IC₅₀ | 95% Confidence |
| | nM | Range | nM | Range | nM | Range |
| PARP1 | 21 | 17-25 | >3000 | N.D. | 2639 | 1927-3654 |
| PARP2 | 28 | 23-33 | 1015 | 789-1305 | 30.3 | 23.5-38.6 |
| PARP3* | 32 | 30-34 | >3000 | N.D. | >3000 | N.D. |
| PARP4bc* | 35 | 33-37 | >3000 | N.D. | >3000 | N.D. |
| PARP5cat | 2520 | 2320-2720 | >3000 | N.D. | >3000 | N.D. |
| PARP6 | 180 | 130-230 | 775 | 557-1084 | >3000 | N.D. |
| PARP7^{†} | 14 | 12-16 | 13.7 | 10.3-18.2 | 11.6 | 10.2-13.2 |
| PARP8 | 4700 | 3700-5700 | >3000 | N.D. | >3000 | N.D. |
| PARP10 | 860 | 560-1160 | 179 | 107-300 | >3000 | N.D. |
| PARP11 | 460 | 440-480 | 134 | 90.3-201 | 1971 | 1652-2363 |
| PARP12cat | >3000 | N.D. | 762 | 536-1088 | 716 | 639-802 |
| PARP14wwe | >10000 | N.D. | 1044 | 657-1694 | >3000 | N.D. |
| PARP15cat | 2000 | 1700-2300 | 770 | 640-927 | >3000 | N.D. |
| PARP16^{†} | 4300 | 3300-5300 | 1056 | 608-1877 | >3000 | N.D. |

PASTA showed that the addition of the propynyl group at the C-6 position of Example 10 dramatically improved its selectivity for PARP7 over PARP1 and PARP2 compared to Phthal01. To gain insight into this improved selectivity, we used an induced-fit docking (IFD) approach to predict the binding mode of Example 10 to a homology model of PARP7 (see Methods). IFD showed that the propynyl group at the C-6 position of Example 10 occupies the hydrophobic cavity, the entrance to which is controlled by the isoleucine (Ile631, human PARP7 numbering) gatekeeper in PARP7 (FIG. 1C). Overlay of the crystal structure of PARP2 bound to olaparib (PDB: 4TVJ) shows that the glutamate (Glu558, human PARP2 numbering) gatekeeper prevents access to the hydrophobic cavity, and thus we predict that it would clash with the C-6 propynyl group of Example 10. Taken together these modeling studies support the notion that Example 10 achieves selectivity for PARP7 over PARP2 (and by extension all PARylating PARPs) by exploiting the hydrophobic cavity in PARP7.

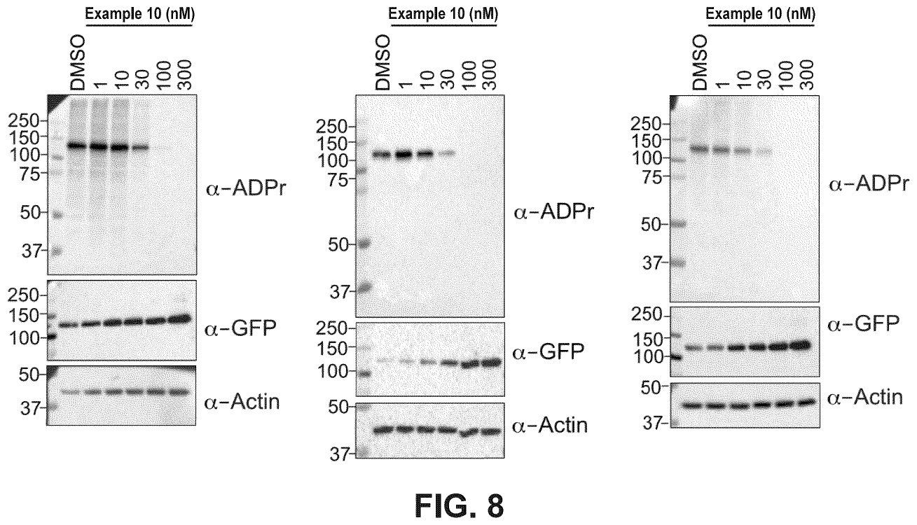

We next sought to determine if Example 10 inhibits the catalytic activity of PARP7 in cells. In previous studies we showed that that overexpression of GFP-PARP7 in HEK 293T cells leads to MARylation of PARP7, which can be assessed by western blot using an antibody that recognizes MARylation. We found that treatment of GFP-PARP7 expressing HEK 293T cells with Example 10 lead to a dose-dependent decrease in PARP7 MARylation (EC₅₀=8 nM) (FIG. 1D, FIG. 1E, and FIG. 8). These results not only show that Example 10 is active in cells, but also provide evidence that the MAR signal detected on PARP7 is due to auto-MARylation and not trans-MARylation by another PARP. We also observed that Example 10 increases GFP-PARP7 protein levels in a dose dependent manner. This is consistent with our previous studies using Phthal01, as well as studies using a catalytically inactive PARP7 mutant.

### Example 10 Validates the Necessity of the Catalytic Activity of PARP7 for its Repressor Function on AHR Activity

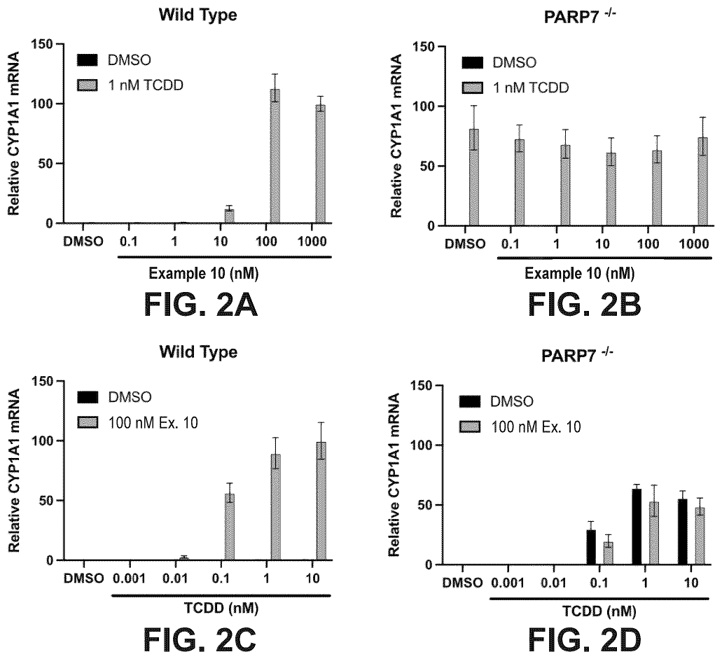

Having established that Example 10 inhibits PARP7 catalytic activity in cells, we next sought to determine the effects of Example 10 on endogenous PARP7 function. For these initial studies, we focused on the role of PARP7 in ligand-mediated AHR transcriptional regulation in mouse embryonic fibroblasts (MEFs). We treated wild type (WT) MEFs with increasing concentrations of Example 10 in the presence or absence of the synthetic AHR agonist TCDD, and subsequently measured the expression of a major AHR target gene. CYP1A1, by quantitative reverse transcription PCR (RT-qPCR). Treatment of WT MEFs with Example 10 in the presence of 1 nM TCDD dramatically increased CYP1A1 mRNA levels in a dose-dependent manner (saturating effects observed at 100 nM Example 10) (FIG. 2A). In the absence of TCDD. Example 10 had no effect on CYP1A1 mRNA levels (FIG. 2A). Likewise, TCDD treatment alone did not induce CYP1A1. In contrast to WT MEFs, PARP7^{-/-} MEFs treated with 1 nM showed a robust induction of CYP1A1 (FIG. 2B). Example 10 treatment did not further enhance the effects of TCDD treatment on CYP1A1 mRNA levels in PARP7^{-/-} MEFs (FIG. 2B). Treatment of WT MEFs with increasing concentrations of TCDD in the presence of 100 nM Example 10 (saturating dose) lead to a strong, dose-dependent induction of CYP1A1 mRNA (FIG. 2C). In PARP7^{-/-} MEFs TCDD treatment alone increased the levels of CYP1A1 in a dose-dependent manner (FIG. 2D). Treatment of PARP7^{-/-} MEFs with 100 nM Example 10 did not further increase TCDD-mediated CYP1A1 induction (FIG. 2D). Our results with Example 10 are congruent with recent studies showing that treatment with RBN-2397, or replacement of endogenous PARP7 with a catalytically inactive variant (H532A PARP7), profoundly enhanced TCDD-mediated AHR transcription. Taken together, these results strongly support the hypothesis that PARP7-mediated MARylation represses AHR activity in a TCDD-dependent manner.

### Example 10 Synergistically Induces IFN-f Expression in the Presence of Cytosolic NA-Sensor Ligands

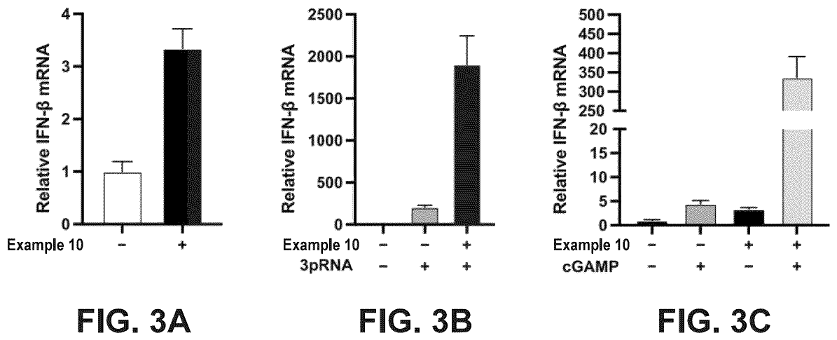

Replacement of WT PARP7 with a catalytically inactive mutant (H532A PARP7) induces the IFN-I, IFN-β. Consistent with these studies, treatment of MEFs with 100 nM Example 10 alone induced a 3-fold increase in the IFN-I IFN-β mRNA as determined by RT-qPCR (FIG. 3A). Strikingly, when MEFs were stimulated with the RIG-I agonist 3pRNA we observed a synergistic induction of IFN-f mRNA (FIG. 3B). Similar results were obtained with the STING agonist cGAMP (FIG. 3C). Our results support the hypothesis that the repressor function of PARP7 on IFN-I expression is dependent on its catalytic activity.

Structurally Distinct PARP7 Inhibitors with Non-Intersecting PARP Family-Wide Selectivity Profiles Induce IFN-I Signaling in Cancer Cells

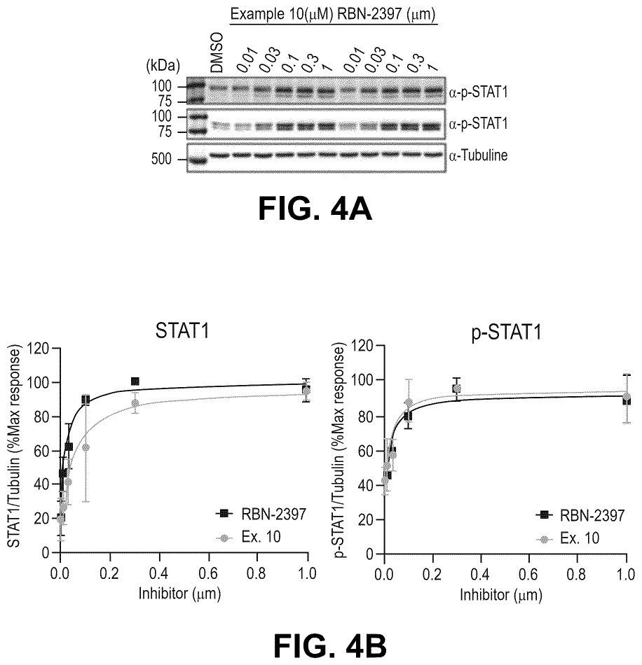

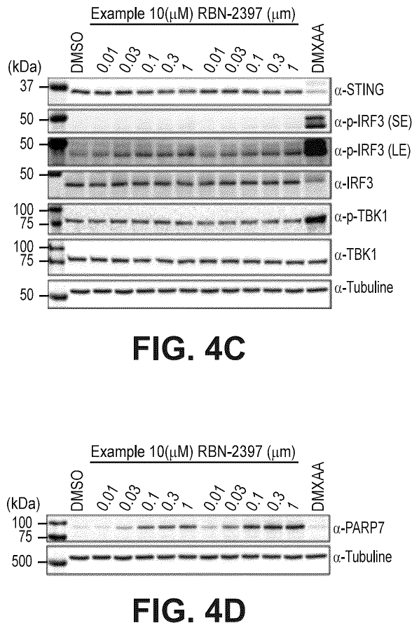

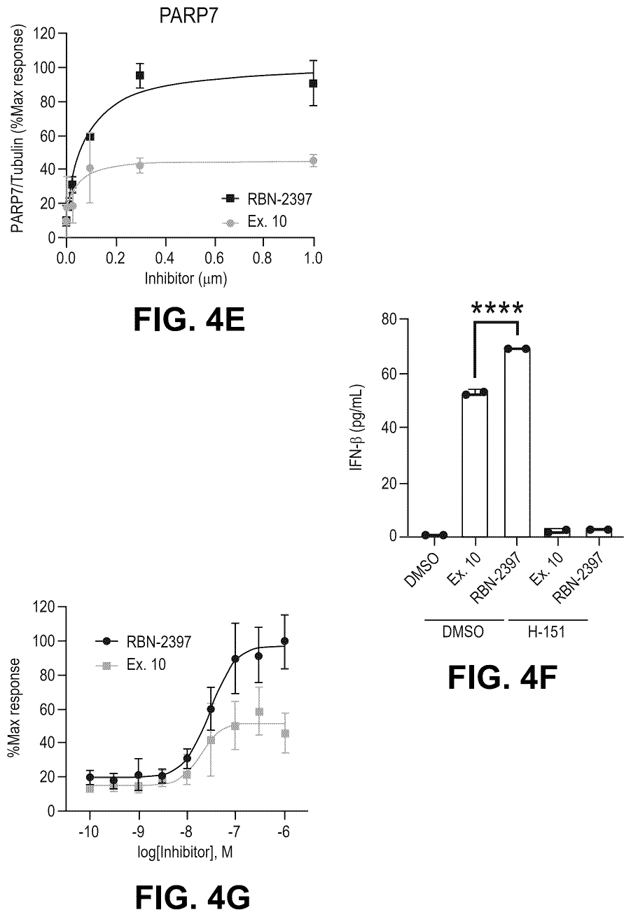

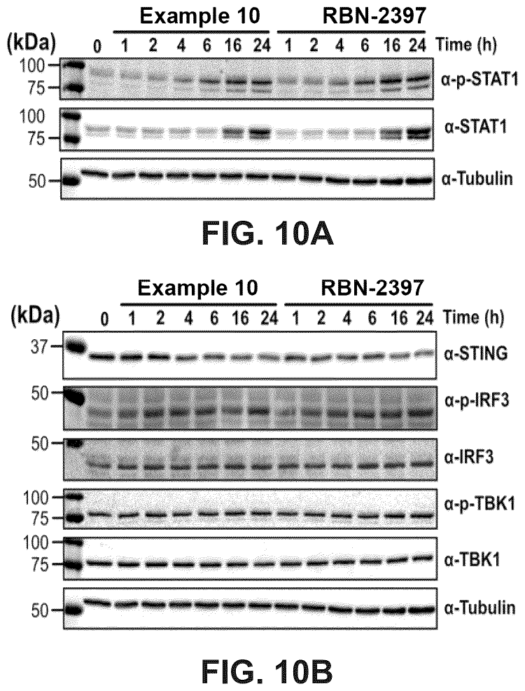

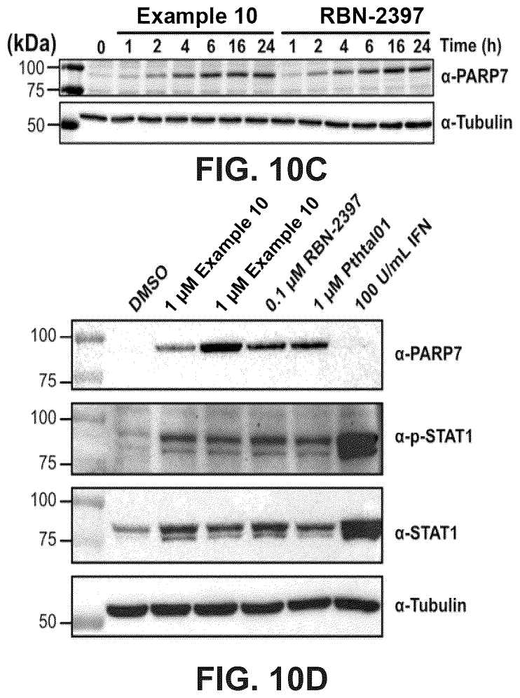

Treatment of cancer cells (e.g., CT-26 and NCI-H1373) with RBN-2397 induced IFN-β expression and downstream signaling, suggesting that inhibition of PARP7 catalytic activity is responsible for this effect. Given that other PARP family members have been implicated in IFN-I signaling (e.g., PARP1/2 and PARP11), we reasoned that the non-overlapping selectivities of Example 10 and RBN-2397 across the PARP family would prove advantageous in studies aimed at validating PARP7 inhibition as the sole driver of IFN-β signaling in cancer cells. To evaluate the effects of these PARP7 inhibitors on IFN-β signaling in CT-26 cells, we monitored changes in STAT1 and STAT1 Tyr701 phosphorylation (pSTAT1) by western blot. Treatment of CT-26 cells with increasing concentrations of either Example 10 or RBN-2397 for 16 h dose-dependently increased STAT1 and pSTAT1 levels (FIG. 4A). Quantification of the STAT1 and pSTAT1 western blot data showed a nearly indistinguishable response between RBN-2397 and Example 10 (FIG. 4B). Moreover, the increase in STAT1 and pSTAT1 by 300 nM (saturating dose) Example 10 and RBN-2397 showed the same time dependence; noticeable increases in pSTAT1 and STAT1 occurred at 4 h and 16 h, respectively (FIG. 10A). Together, these results provide evidence the induction of IFN-β signaling in CT-26 cells by Example 10 and RBN-2397 is due to PARP7 inhibition, and not inhibition of other PARP family members.

Previous studies showed that RBN-2397-mediated induction of IFN-β is dependent on a functional STING pathway; however, whether PARP7 acts upstream or downstream of STING was not clear. STING activation leads to auto-phosphorylation (Ser172) and activation of TBK1, followed by TBK1-mediated phosphorylation of IRF3 on Ser396. This leads to IRF3 dimerization and nuclear translocation where it regulates the transcription of IFN-I and other cytokines. Activation of STING by various ligands promotes its ubiquitylation and subsequent degradation by autophagy, attenuating the IFN-I response. Indeed, we found that the mouse-specific STING agonist DMXAA decreased substantially STING levels and induced the appearance of a higher molecular weight band (FIG. 4C). In contrast, both PARP7 inhibitors only decreased slightly STING levels in a dose-dependent (FIG. 4C) and time-dependent (FIG. 10B) manner. Although this is inconsistent with canonical STING activation, it is possible that PARP7 inhibition activates STING in a non-canonical manner. We therefore looked at pTBK1 and pIRF3. In contrast to DMXAA, which induced robustly both pTBK1 and pIRF3. PARP7 inhibition did not induce pTBK1 and only increased slightly pIRF3 (FIG. 4C). Taken together, these results suggest that PARP7 inhibition regulates IFN-β signaling downstream of TBK1/IRF3.

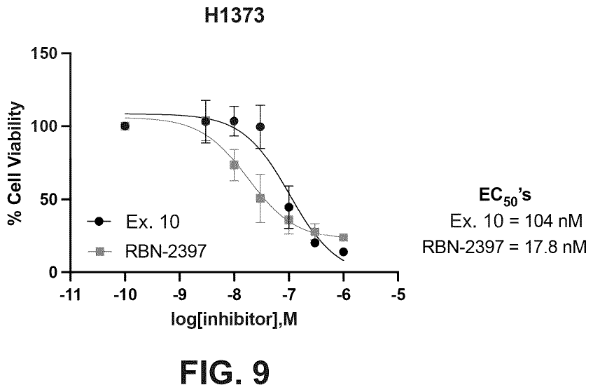

Activation of the IFN-I-mediated STAT1 signaling pathway in certain cancer cells can lead to a loss of viability in a cell-autonomous manner. Indeed, RBN-2397 was shown to decrease the viability of NCI-H1373, but not CT-26 cells. Consistent with this finding, Example 10 dose-dependently decreased the viability of NCI-H1373 cells (FIG. 9), but did not decrease viability in CT-26 cells. The EC₅₀ for Example 10 on NCI-H1373 is 104 nM whereas we found that the ECs for RBN-2397 is 17.8 nM. Why Example 10 is ˜6-fold less potent than RBN-2397 is not clear considering their similar potencies against PARP7 in vitro and their similar efficacies in inducing IFN-I in CT-26 cells. Nevertheless, these results strengthen the notion that the loss of PARP7 catalytic activity decreases viability in NCI-H1373.

PARP7 Inhibitors Increase Endogenous PARP7 Levels, and the Extent of this Increase Correlates with The Magnitude of the IFN-I Transcriptional Response

Since we observed an increase in the protein levels of ectopically expressed GFP-PARP7 upon treatment with Example 10, we wondered if Example 10 and RBN-2397 increased the levels of endogenous PARP7 in CT-26 cells. In the absence of inhibitor, the levels of endogenous PARP7 were nearly undetectable; however, increasing concentrations of Example 10 and RBN-2397 increased PARP7 levels (FIG. 4D). Quantification of the western blot data revealed that RBN-2397 caused an unexpected 2-fold increase in PARP7 protein abundance compared to Example 10 at saturating doses (FIG. 4E). Given this difference, we asked if the increase in PARP7 protein levels occurred on similar timescales. The time-dependent increase in PARP7 protein levels was similar for both inhibitors, with maximal PARP7 protein levels occurring ˜16 h (FIG. 10C).

Although both PARP7 inhibitors induced similar levels of pSTAT1/STAT1 (FIG. 4B), we wondered if the levels of PARP7 inhibitor-induced secreted IFN-β were different between Example 10 and RBN-2397. Using ELISA, we found that RBN-2397 increased significantly the levels of secreted IFN-β to a greater extent than Example 10 at saturating doses (FIG. 4F). As expected, the PARP7 inhibitor-induced induction of IFN-β was completely blocked by the covalent STING inhibitor H-151 (FIG. 4F). Since PARP7 induces IFN-β and given that many PARPs are interferon stimulated genes (ISGs), we wondered if IFN-β alone increased PARP7 levels. In contrast to PARP7 inhibitors, recombinant IFN-β did not increase PARP7 levels despite strong induction of pSTAT1/STAT1 (FIG. 10D); hence, the PARP7-inhibitor induced increase in PARP7 levels is independent of IFN-β.

To determine if the higher levels of RBN-2397-induced IFN-β compared to Example 10 correlated with higher levels of IFN-β-dependent transcription, we generated an interferon-sensitive response element (ISRE)-firefly luciferase (Lucy-CT-26 cell line using lentiviral transduction. ISRE-Luc have been used in various cell lines as a convenient reporter of IFN-I-dependent transcription. We treated ISRE-Luc)-CT-26 cells with increasing concentrations of either Example 10 or RBN-2397 for 16 h, and then measured Luc-generated luminesce in lysates. Intriguingly, while the EC₅₀ for the two inhibitors was similar, the magnitude of the response was greater (˜2-fold) for RBN-2397 compared to Example 10 at saturating doses (FIG. 4G). The extent of the ISRE increases is well correlated with the extent of the PARP7 protein level increases in the presence of saturating inhibitors. Taken together, these results possibly hint at additional mechanisms—beyond catalytic inhibition—by which PARP7 inhibitors regulate IFN-I signaling.

### PARP7 Inhibition Increases PARP7 Abundance in the Nucleus

To gain insight into how PARP7 inhibitor-mediated increases in PARP7 protein levels could influence IFN-I transcriptional output, we next determined (i) the endogenous PARP7 localization, which is currently unknown, and (ii) if PARP7 localization changes upon inhibitor treatment, as we have observed for other PARPs. Unfortunately, there is only one validated PARP7 antibody, which in our hands did not work for immunofluorescence (IF). We therefore sought a strategy in which endogenous PARP7 could be replaced with an epitope-tagged variant. We envisioned that the epitope tag would be small enough so as to not perturb endogenous PARP7 function or localization.

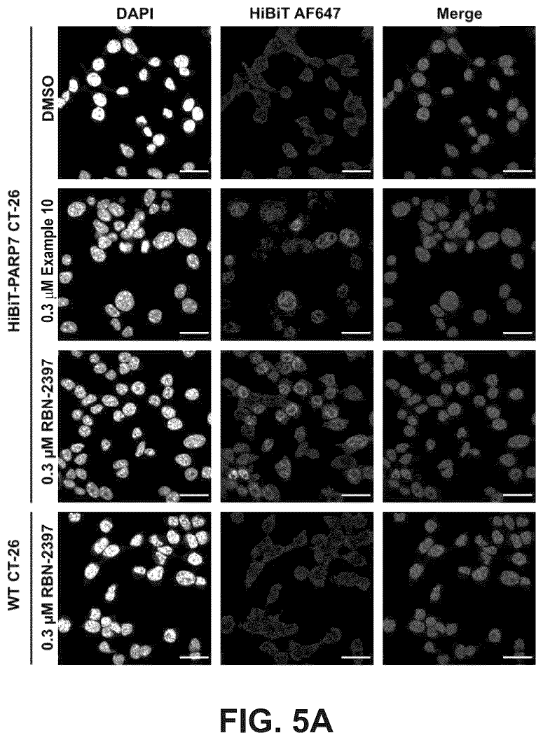

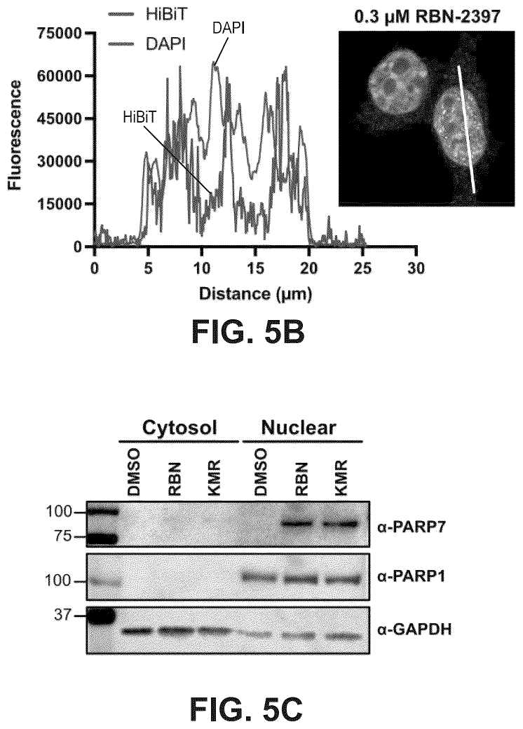

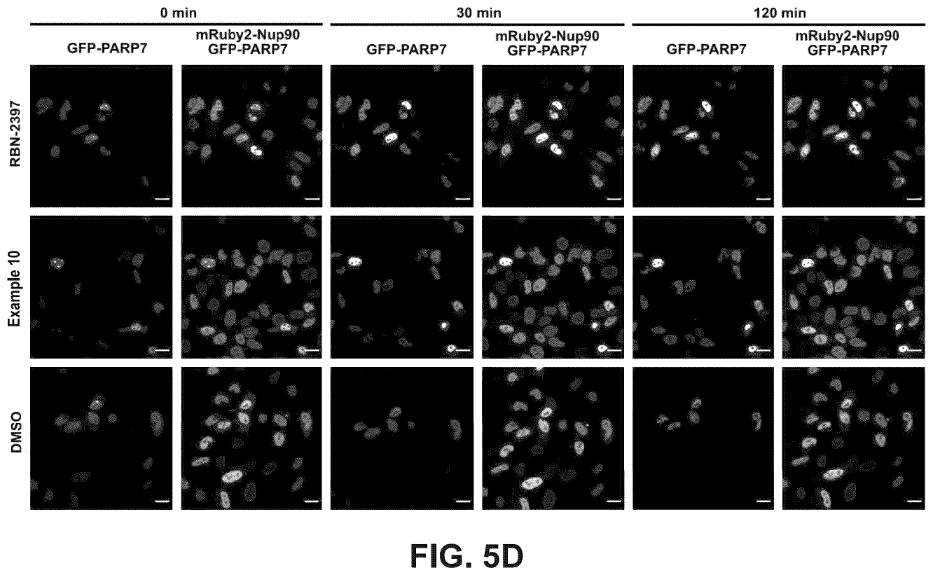

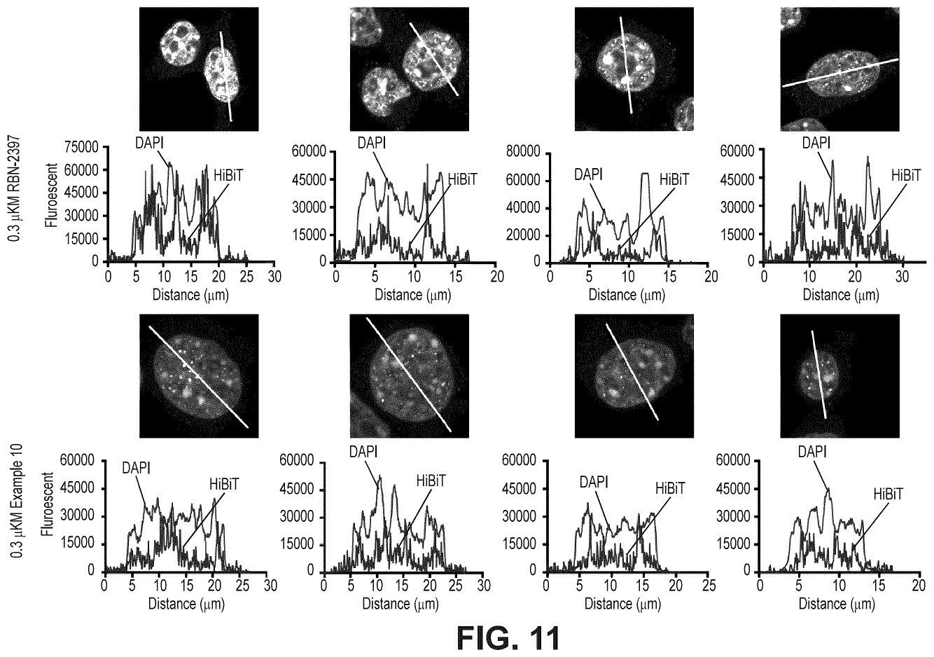

Recently, a versatile, small epitope tag, termed HiBiT, was developed. Importantly, an antibody against HiBiT has been used in IF applications. Another feature of the HiBiT is that it binds with high affinity to LgBiT, the binding of which reconstitutes a luminescent protein akin to Nano luciferase. The small size of the HiBiT allows for efficient knock-in using CRISPR/Cas9. We therefore generated CT-26 cells in which endogenous PARP7 was replaced with HiBiT-PARP7. HiBiT-PARP7-CT-26 cells were treated with 300 nM Example 10 or RBN-2397, or vehicle control (DMSO) for 18 h. HiBiT-PARP7 localization was assessed by IF using an anti-HiBiT antibody. In the absence of PARP7 inhibitors, we could not detect HiBiT-PARP7, preventing us for asking if PARP7 localization changes upon inhibitor treatment (FIG. 5A). In the presence of Example 10 and RBN-2397, a nuclear HiBiT signal that colocalized with DAPI could be detected (FIGS. 5B, 5C, and 11). The HiBiT-PARP7 signal was stronger for RBN-2397 compared to Example 10, consistent with western blot analysis of endogenous, untagged PARP7. Importantly, no signal was detected in WT CT-26 cells treated with 300 nM RBN-2397, demonstrating the specificity of the HiBiT-PARP7 signal in HiBiT-PARP7-CT-26 cells (FIG. 5A). To confirm our localization studies, we biochemically fractionated lysates from WT CT-26 cells treated with or without PARP7 inhibitors (300 nM). Using western blot with a validated PARP7 antibody, PARP7 was not detected in either the cytoplasmic or nuclear fractions; in contrast. PARP7 was detected robustly in the nuclear fraction (FIG. 5C). A very faint signal could be detected in the cytoplasmic fraction (FIG. 5C). Together, these results show that in CT-26 cells, the PARP7-inhibitor complex is localized predominately to the nucleus.

Because we could not detect HiBiT-PARP7 in the absence of PARP7 inhibitors, we could not determine if PARP7 inhibitors altered the localization of PARP7. We therefore turned to live-cell imaging experiments using ectopically expressed GFP-PARP7 in HeLa cells, which are commonly used for these types of studies. HeLa cells were transfected with GFP-PARP7 as well as mRuby2-Nup90, which was used as a nuclear marker. These transfected cells were treated with 300 nM RBN-2397, Example 10, or DMSO and localization was monitored via live-cell imaging over a period of 2 h. In the absence of PARP7 inhibitors. GFP-PARP7 localized to the nucleus, consistent with our IF studies using HiBiT-PARP7. In some cells, GFP-PARP7 was found in large puncta in the nucleus (FIG. 5D). Within 30 min of PARP7 inhibitor treatment, these nuclear puncta disappeared, and GFP-PARP7 was instead localized in a diffuse, pan-nuclear pattern (FIG. 5D). The pan-nuclear localization of GFP-PARP7 in the presence of inhibitors is similar to the localization of GFP-tagged, catalytically inactive PARP7. Taken together, these results suggest that PARP7 catalytic activity controls its localization in cells.

### Discussion

Much attention—both in terms of fundamental research and therapeutic development—has been given to PARylating PARPs (i.e., PARP1, PARP2, and PARP5a/b (Tankyrase1/2) in large part because multiple classes of potent and selective inhibitors based on distinct chemotypes are available. While genetic tools, such as RNAi and most recently CRISPR/Cas9, have been powerful means to prosecute the biology of the lesser known MARylating PARPs, selective small molecule inhibitors of MARylating PARP offer unique advantages over genetic strategies, chiefly among them is their ability to directly assess the functional role of PARP catalytic activity in cells. Compared to PARylating PARPs, there is a dearth of inhibitors selective for individual MARylating PARPs, which has hampered the studies focused on elucidating the physiological and pathophysiological roles of their catalytic activity. Previously we uncovered a hydrophobic cavity in MARylating PARPs, the accessibility of which is controlled by a hydrophobic gatekeeper amino acid. The hydrophobic cavity is not accessible in PARylating PARPs because a glutamate in the gatekeeper position blocks the entrance to the hydrophobic cavity. In this study, we exploited differences in the gatekeeper position between PARylating PARPs and MARylating PARPs to rationally convert a pan-PARP inhibitor into a selective PARP7 inhibitor, Example 10.

Example 10 is structurally distinct from the previously described PARP7 inhibitor RBN-2397. Example 10 is based on a phthalazinone scaffold whereas RBN-2397 is based on a pyridazinone scaffold. When profiled in our PARP inhibitor screening platform, PASTA, Example 10 and RBN-2397 inhibited PARP7 with similar efficacy, but showed different selectivity across the family. Of particular note, Example 10 showed better selectivity for PARP7 compared to the PARylating PARP, PARP2. Modeling suggests that the improved PARP7 to PARP2 selectivity is due to a greater engagement of the hydrophobic cavity by Example 10 versus RBN-2397.

Example 10 was used to investigate the role of PARP7 catalytic activity in regulating AHR transcription and IFN-I signaling driven by RIG-1 and STING in MEFs. Example 10 synergized with an AHR agonist to enhance AHR-mediated transcription. Similarly. Example 10 synergized with RIG-LISTING ligands to induce the expression the IFN-I, IFN-β. These results described here, together with previous studies using RBN-2397 and a catalytically inactive PARP7 mutant, support the hypothesis that the catalytic activity of PARP7 acts as a repressor AHR and RIG-I/STING-mediated IFN-I signaling. In the case of AHR, it was shown that PARP7 can MARylate AHR in vitro and in cells: however, whether AHR MARylation is the means by which PARP7 catalytic activity regulates AHR-mediated transcription remains to be determined. In the case of RIG-I/STING-mediated IFN-I signaling, PARP7 was shown to MARylate TBK1 in vitro, but studies showing directly MARylation of TBK1 are lacking. Using a combined chemical genetics and proximity labeling approach, we identified several direct targets of PARP7, several of which have known roles in IFN-I signaling. We did identify TBK1 as a PARP7 interacting partner, but not as a MARylation target. One possible reason for this is that MARylation of TBK1 by PARP7 is context dependent (for example, during cytoplasmic NA-sensor stimulation).

Curiously, we found that the RBN-2397 and Example 10 increased endogenous PARP7 protein levels in CT-26 cells. Previous studies found that a catalytically inactive GFP-PARP7 mutant is expressed at a higher level than WT GFP-PARP7. Additionally, the expression levels of WT GFP-PARP7 could be increased using MG132. Our results presented here, together with the aforementioned studies, support the hypothesis that PARP7 stability is regulated by its catalytic activity in a ubiquitin-dependent manner. How catalytic activity of PARP7 regulates its stability is not clear; however, we and others have identified several ubiquitin E3 ligases that interact with PARP7.

Finally, we found that the magnitude of IFN-I-dependent transcription correlated with the levels of PARP7 protein levels in the presence of PARP7 inhibitors. While this is corelative at the moment, one intriguing possibility is that the PARP7 inhibitor-PARP7 complex drives IFN-I signaling.

### Biological Example 5: Methods for the Figures and for Biological Example 4 Cell Culture

All cells were cultured at 37° C. with 5% CO₂. HEK293T cells were cultured in DMEM+1× Glutamax+10% FBS. CT-26 and NCI-H1373 cells were cultured in RPMI-1640+10% FBS. ISRE-Luc expressing CT-26 cells were cultured in RPMI-1640+10% FBS+20 μg/ml puromycin (Sigma).

### Stable Cell Line Production

ISRE-Luc CT-26 cells were produced by lentiviral transduction. Briefly, 400.000 CT-26 cells in 800 μl of media were incubated with 200 μl of ISRE Luciferase Reporter Lentivirus (BPS Biosciences, #79824) with 8 μg/ml polybrene. The mixture was centrifuged at 800 g for 30 min at 25° C. Media was removed and cells were plated in 2 ml of fresh media. 48 h post transduction, cells were selected for with 20 μg/ml puromycin. Following selection, cells were continuously cultured win 20 μg/ml puromycin as described above.

HiBiT-PARP7 KICT-26 cells were generated by Synthego via CRISPR/Cas9 Knock-in (KI) of SEQ ID No. 1: GTGAGCGGCTGGCGGCTGTTCAAGAAGATTAGC to the PARP7 N-Terminus. The Knock-in cell pool was cultured as previously described for CT-26 cells.

### PASTA Biochemical PARP Screening Assay

N-Terminal His-tagged PARPs and SRPK2 were expressed and purified as previously described (Carter-O'Connell et al., J. Am. Chem. Soc. 2014, 136, 14, 5201-5204.) Proteins were quantified against an in-gel standard curve of Bovine Serum Albumin (Bio-Rad) using ImageLab (BioRad).

PARPs 1, 2, 5cat, 6, 8, 10, 11, 12cat. 14wwe, 15cat plate assays were performed as described (Kirby et al, STAR Protocols, 2021, 2(1), 100344.). Briefly, 50 μl of 1 μM SRPK2 in PARP Buffer for reaction (PBR) (50 mM HEPES pH 7.5, 100 mM NaCl, 4 mM MgCl₂, 0.2 mM TCEP) was added to wells of a Nickel-NTA coated 96-well plate (Thermo Scientific) and incubated at RT for 1 h. The solution was removed, and the plate was washed 3 times with PBST (1×PBS, 0.1% Tween-20), once with 1×PBS, and once with PBR. Each wash was incubated for 3 min. After washing, varying concentrations of each inhibitor was pre-incubated with 20 μM (2×) 6-a-NAD⁺ (prepared as described) (Carter-O'Connell et al., J. Am. Chem. Soc. 2014, 136, 14, 5201-5204.) 25 μl of the 2×6-a-NAD⁺/inhibitor mixture was added to the plate with 25 μl of 2×PARP. For PARP1 and PARP2, 0.1 mg/ml Activated DNA (Sigma) was supplemented. The reaction mixture was incubated at 30° C. for 1 h. The plate was then washed 3 time with PBST and once with PBS. The wells were subjected to a 50 μL click reaction consisting of 100 μM biotin-PEG3-Azide (Click Chemistry Tools), 100 μM Tris[(1-benzyl-1H-1,2,3-triazol-4-yl)methyl] amine (TBTA) (Click Chemistry Tools), 1 mM CuSO4 and 1 mM TCEP in 1×PBS at RT for 30 min. The plate was then washed 3 times with PBST before blocking in 100 μl of 1% Milk (Carnation) in PBST at RT for 30 min. The plate was again washed 3 times with PBST and once with PBS before incubation with Strep-HRP (0.05 ng/μl Strep-HRP (Fisher Scientific), 300 ng/μl BSA, 1×PBS) for 30 min at RT. The plate was washed 3 time in PBST and once with PBS before development with QuantaRed (Pierce) per manufacturer directions. The plate was immediately read for fluorescence (Excitation 550 nm, Emission 620 nm) on Spectra Max i3 (Molecular Devices) following development. Three parameter nonlinear regression was performed with GraphPad Prism 9 to obtain IC₅₀ values.

For PARP3, PARP4bc, and PARP16, 200-350 ng of PARP was directly adhered to nickel coated 96-well plates for 60 min at 25° C. in PBR PARP3 was activated by Dnick 5'P as previously described (Langelier, Marie-France et al. Nucleic acids research vol. 42, 12 (2014): 7762-75.). The plate was washed three times with PBST, once with PBS, and once with PBR. Varying concentrations of inhibitor were pre-incubated with NAD⁺ and added to the plate (final NAD⁺ concentration: 100 μM for PARP3, PARP4bc; 400 μM for PARP16). The plate was incubated at 30° C. for 60 min, then washed three times with PBST before blocking with 5% Milk in PBST. The plate was washed three times with PBST and then incubated with 1:5000 (PARP3) or 1:25000 (PARP4bc. PARP16) Poly/Mono-ADP Ribose rabbit monoclonal antibody (Cell Signaling Technology) in PBST with 2% BSA and 0.05% NaN₃ for 30 min at 25° C. The plate was washed three times with PBST before incubation with 1:5000 (PARP3) or 1:25000 (PARP4bc. PARP16) anti-Rabbit HRP (Jackson ImmunoResearch) in 5% Milk in PBST for 30 min at 25° C. The plate was then washed three times with PBST and once with PBS before developing as described previously.

GST-tagged PARP7 was expressed and purified as previously described (Hutin et al., 2018). Proteins were quantified by an in-gel standard curve of Bovine Serum Albumin (Bio-Rad).

For PARP7, 250 ng of PARP7 was adhered to glutathione coated 96-well plates (Thermo Scientific) in PBR for 60 min at RT. The plate was washed three times with PBST (1×PBS+0.1% Tween-20), once with PBS, and once with PBR (50 mM HEPES pH 7.5, 100 mM NaCl, 4 mM MgCl₂, 0.2 mM TCEP). After washing, varying concentrations of each inhibitor was pre-incubated with 20 μM (2×) 6-a-NAD⁺. The solution was added to the plate, diluted to 1× in PBR, and incubated at 30° C. for 60 min. The plate was washed three times with PBST and once with PBS before performing click conjugation (100 μM biotin-PEG3-Azide, 100 μM Tris[(1-benzyl-1H-1,2,3-triazol-4-yl)methyl] amine (TBTA), 1 mM CuSO4, 1 mM TCEP, 1×PBS) for 30 min at RT. The plate was then washed three times with PBST and blocked with 1% Milk (Carnation) in PBST. The plate was subsequently washed three times in PBST and once in PBS before incubation with Strep-HRP (300 ng/μl BSA, 0.05 ng/μl Strep-HRP (Jackson ImmunoResearch), 1×PBS) for 30 min at RT. The plate was washed three time in PBST and once in PBS before development with QuantaRed Enhanced Chemifluorescent HRP Substrate as described previously.

### Molecular Modeling

Due to the lack of experimental structure of PARP7, a homology model was built based on the sequence alignment. 6V3W is the PDB code of PARP12 complex with a small molecule RBN-2397 and used as the template to develop the homology model of the PARP7 catalytic domain. A pharmacophore model was developed and used to place the RBN-2397 and Example 10 efficiently in the pocket followed by binding affinity assessment in the process of docking. Both the pharmacophore development and docking were carried out by MOE software package from CCG (https://www.chemcomp.com/).

### Western Blotting

CT-26 cells at 75% confluency were treated with PARP7 inhibitors from 0-1 μM for 16 h for the dose-response or at 300 nM for the time-course (0-24 h). As a positive control, cells were treated with DMXAA (10 μg/ml) for one hour. At the end of treatment, cells were washed once with cold PBS and the plate was frozen in −80° C. prior to lysis. For lysis, the plate was thawed on ice, and lysed using a cytosolic lysis buffer (50 mM HEPES pH 7.4, 150 mM NaCl, 1 mM MgCl2, 1% triton X-100 supplemented with fresh 100 μM TCEP, 1× protease inhibitor (Roche), 1× phosphatase inhibitors (Sigma cocktail 2&3), and 30 μM Pan-PARP inhibitor Pthtal01. Lysates were clarified by centrifugation at 14,000 rpm at 4° C. for 10 minutes. Supernatants were transferred into a new tube and protein concentration was determined using the Bradford assay (Bio-Rad). 4× sample buffer (40% glycerol, 200 mM Tris-Cl (pH 6.8), 8% SDS, 4% β-mercaptoethanol, 0.02% bromophenol blue) was added to normalized lysates and samples were boiled at 95° C. for 5 min. Total protein was separated on a 10% SDS-PAGE gel, transferred to nitrocellulose membrane using Trans-Blot Turbo Transfer System (BioRad), followed by blocking with 5% milk (Carnation) in TBST. The blots were probed overnight at 4° C. against the following antibodies (Cell Signaling Technology): STING (1:000, D2P2F). Rig-I (1:1000, D14G6), phospho-STAT1 (Tyr701) (1:000, D4A7), STAT1 (1:000, 9172), phospho-IRF3 (Ser396) (1:1000, 4D4G), IRF3 (1:1000, D6I4C), Phospho-TBK1 (Ser172) (1:1000, D52C2), TBK1 (1:1000, D1B4), and tubulin (1:2000, DM1A). PARP7 was detected using an in-house mouse anti-PARP7 antibody (Rasmussen 2021). After PBST washes, blots were incubated with a goat anti-rabbit (1:10000, Jackson Immuno Research Labs) or goat anti-mouse (1:5000, Invitrogen) HRP-conjugated secondary antibody in 5% milk in TBST for 1 hour at room temperature. Blots were washed with TBST and developed using SuperSignal™ West Pico or Femto chemiluminescent substrate (Thermo Scientific) and imaged on a ChemiDoc Gel Imaging System (BioRad).

MEFs isolated from C57BL/6 mice were plated at a density of 1.0×10⁵ cells per ml in a 6-well plate. The following day, cells were treated with DMSO, 100 nM RBN-2397 or 100 nM KMR for 6 hours. Two wells were pooled, and pellets were resuspended in 20 ml of PBS-T [phosphate buffered saline (PSB) containing 0.1% Tween20]. The cells were lysed in 200 μl of SDS lysis buffer (1% SDS, 100 mM Tris, pH 7.5, 200 mM NaCl, 10% glycerol) supplemented with 1× protease inhibitors cocktail (Merck) and 2 mM DTT. Samples were sonicated using a Bioruptor at low intensity for two 30 seconds on/off cycles then centrifuge at 20,000×g for 10 min at 4° C. Protein concentration was determined, and sample diluted with Laemmli buffer and incubated at 95° C. for 5 min. 25 μg of protein were separated by SDS-PAGE and transferred to a PVDF membrane. Membranes were blocked for 1 h at room temperature in 5% non-fat milk dissolved in TBS containing 0.1% Tween20 (TBS-T). Membranes were then incubated overnight at 4° C. with an in-house generated anti-PARP7 (Rasmussen, 2021), 1:8000 dilution of anti-AHR (Enzo Life Sciences SA210; lot #03011639) followed by incubation with the appropriate secondary antibodies. PVDF membranes were stripped and incubated with anti-β-actin antibody 1:4000 (Sigma-Aldrich; A-2228).

### RNA Extraction and Gene Expression Analysis

CT26 cells, MEFs isolated Parp7^{-/-} or Parp7^{-/-} mice (MacPherson, 2013 #2606) were plated at a density of 1.0×10⁵ cells per ml in a 12-well plate. The following day, cells were treated for 24 hours with DMSO, 2,3,7,8-tetrachlorodibenzo-p-dioxin (TCDD) (Accustandard: New Haven, CT, USA). KMR or TCDD+KMR at the doses indicated in the figure captions. For the 3pRNA and cGAMP studies, Parp7^{-/-} MEFs were plated at a density of 1.0×10⁵ cells per ml in a 12-well plate. The following day, the cells were transfected with 100 ng/ml 3p-hpRNA (Invivogen) or 5 mg/ml 2′3′-cGAMP (Invivogen) using Lipofectamine 2000 and immediately treated with 100 nM KMR for 4 h. Total RNA was isolated using Aurum™ Total RNA isolation kit (BioRad), and 1 mg of RNA was reverse transcribed using High Capacity cDNA Reverse Transcription Kit according to the manufacturer's instructions (Applied Biosystems). Each RT-qPCR reaction consisted of 0.1 μl forward primer, 0.1 μl reverse primer, 5 μl 2×SsoAdvanced Universal SYBR Green Supermix (BioRad), 1 μl of cDNA and dH₂O to a total volume of 10 μl. The primers used for TATA-binding protein (TBP) were forward SEQ ID No.2: 5′-TTGTACCGCAGCTGCAAAAT-3′ and reverse SEQ ID No.3: 5′-TATATTCGGCGTTTCGGGCA-3′, for PARP7 antisense (AS) were forward SEQ ID No.4: 5′-CTATGCGCGTGTTTTGCAG-3′ and reverse SEQ ID No.5: 5′-TCTGGGTGATGAAGGTCGTAG-3′, for Cytochrome P450 family 1 subfamily A member 1 (CYP1A1) were forward SEQ ID No.6: 5′-TGGTCTCCCTTCTCTACACTCTTGT-3′ and reverse SEQ ID No.7: 5′-ATTTTCCCTATTACATTAAATCAATGGTCT-3′, and for IFNb were forward SEQ ID No.8: 5′-TGGGAGATGTCCTCAACTGC-3′ and reverse SEQ ID No.9: 5′-CCAGGAGTAGCTGTTGTACT-3′. All target transcripts were normalized to TBP and analyzed using the comparative cycle threshold (CT) (ΔΔCT) method.

### ISRE Luciferase Assay

10,000 ISRE-Luc CT-26 or WT CT-26 cells were seeded into wells of a white 96-well plate and grown overnight. Compounds were serial diluted at 1000× concentration in DMSO. For dosing, compound dose response curves were dilute 1:100 in culture media, then added at 1:10 dilution to cell culture. Final DMSO concentration was 0.1%. Cells were incubated in treatment for 18 hours. Following treatment, the plates were developed using ONE-Glo Luciferase Assay system (Promega) per manufacturer directions. The luminescence signal was read on a Spectra Max i3 (Molecular Devices) plate reader with a 2 second integration time. WT CT-26 signal was background subtracted and curves were fit using a four-parameter linear regression in GraphPad Prism 9.

### Interferon-β ELISA

CT-26 cells were seeded into a 6-well plate. After 24 h, the growth media was replaced, and cells were treated with inhibitors. After treatment, growth media was harvested and assayed with PBL Verikine Mouse IFN-β ELISA kit following the manufacturer protocol.

### HiBiT Staining

50,000 HiBiT-PARP7 KI CT-26 or WT CT-26 cells were plated onto coverslips. After 24 hours, the cells were treated with PARP7 inhibitors or DMSO for 18 hours. The cells were then washed once with cold PBS and fixed with 4% PFA in PBS at RT for 10 min. The coverslips were washed three times in PBS over a 5-minute period. The HiBiT antibody (Promega) was then added at 1:1000 dilution in antibody incubation buffer (3% Horse Serum, 0.5% Triton X-100, 0.025% NaN₃, PBS) for 2 hours at RT. The coverslips were washed three times in PBST (PBS, 0.1% Tween-20) for 5 min per wash. Subsequently, the coverslips were incubated with 1:1000 AlexaFluor657 anti-Mouse in antibody incubation buffer for 1 hour at RT protected from light. Finally, the coverslips were counterstained with 1:5000 DAPI in PBS for 10 min before washing three times in PBST. The coverslips were then mounted onto slides with Citifluor CFMR2 Antifade (Electron Microscopy Sciences) and sealed with CoverGrip (Biotium). Imaging was performed on a Ziess LSM880 confocal laser scanning microscope with Airyscan with a 63×oil objective at 2048×2048 resolution DAPI excitation was performed with a 405 nm laser at low (≤1%) laser power. Red fluorophores were excited with a 559 nm laser at 7.5% laser power.

### Cellular Fractionation

CT26 cells were culture as described above. Cells were treated with RBN-2397 or Example 10 for 18 h before harvest. Nuclear-cytoplasmic fractionation was carried out using the NE-PER™ Nuclear and Cytoplasmic Extraction Reagents kit (Thermo Fisher Scientific) according to the manufacturer's protocol. Western blot was performed as described above.

### Live Cell Imaging

Hela cells were seeded and grown in 8 well-chambered glass slides (Lab-Tek, Nunc. Cat No. 177402). Cells were subsequently transfected with 100 ng DNA of each of the plasmid constructs (GFP-PARP7 and mRuby2-Nup50-N-10 (Addgene plasmid 55908)) using jetOptimus DNA transfection reagent (Polyplus. Cat No. 117-07), according to manufacturer protocol. 10 h after transfection, media containing transfection mixture was removed. Live cell imaging was performed on the Olympus FV1200 microscope equipped with an environmental box. Cells were kept at 37° C. and 5% CO2 during the whole course of imaging. All images were taken with the 63× oil objective NA=1.35, sequential acquisition. Acquired images were analyzed using ImageJ software.

### Cell Titer-Glo Viability

NCI-H1373 cells were seeded in a white 96-well plate (Corning) at a density of 1,000 cells per well. After 24 hours, cells were treated with PARP7 inhibitors and incubated at 37° C. for 6 days. Cell viability was assessed using CellTiter-Glo (CTG) 2.0 (Promega). After addition of the CTG reagent (following manufacturer protocol), the plate was incubated on an orbital shaker for 15 min (protected from light) at room temperature. Chemiluminescence was measured on a Spectra Max i3 (Molecular Devices), normalized to DMSO-treated wells, and IC₅₀ values were calculated by non-linear regression (three-parameter fit) analysis using Prism 9 (GraphPad).

### Chemistry Methods

### Synthesis of Compound 1B

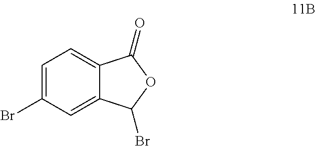 [SMILES: O=C1OC(Br)c2cc(Br)ccc21]

5-bromo-phthalide (2 g, 9.44 mmol) was heated to reflux in carbon tetrachloride (CCl4) with Azobisisobutyronitrile (160 mg, 0.944 mmol) and N-Bromosuccinimide. (1.9 g, 10.7 mmol) for 4 hours until reaction was completed. The reaction was then filtered and the filtrate was concentrated. The crude mixture was then purified via column purification using combiflash ISCO purification system using a mobile phase gradient from 0 to 30% ethyl acetate in hexanes. 2.35 g of compound 1B was obtained (86% yield). ¹H NMR (400 MHz. CDCl3) δ 7.84-7.71 (m, 3H), 7.34 (s, 1H).

### Synthesis of Compound 2B

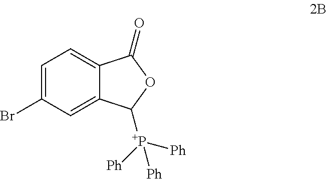 [SMILES: O=C1OC([P+](c2ccccc2)(c2ccccc2)c2ccccc2)c2cc(Br)ccc21]

Under dry conditions, in a clean, oven dried flask, compound 1B (white solid) (2 g, 6.9 mmol) was heated slightly (50° C.) under vacuum pressure with dry triphenyl phosphine (1.81 g, 6.9 mmol) for 30 mins. Triphenyl phosphine and compound 1 were both dried over P2O5 overnight prior to starting the reaction. To the dry starting materials, dry THF was added and the reaction was allowed to stir under reflux overnight. A white precipitate formed quickly after reaction started. The white precipitate, compound 2B, was filtered out and dried under vacuum. (2.6 g, 80% yield) ¹H NMR (400 MHz, CDCl₃) δ 10.29 (s, 1H), 7.88 (ddd, J=19.4, 15.0, 9.1 Hz, 8H), 7.70 (td, J=7.9, 3.7 Hz, 7H), 7.60 (d, J=8.2 Hz, 2H), 7.25 (s, 1H).

### Synthesis of Compound 3B

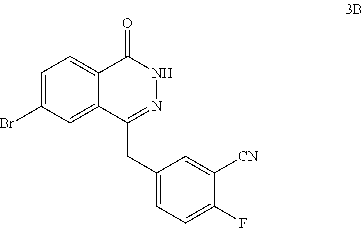 [SMILES: N#Cc1cc(Cc2n[nH]c(=O)c3ccc(Br)cc23)ccc1F]

Compound 2B (0.475 g, 1.0 mmol) and 2-fluoro-5-formylbenzonitrile (0.180 g, 1.2 mmol) were dissolved DCM (4.0 mL), to this was added triethylamine (0.18 mL, 1.3 mmol) and the reaction was stirred overnight. The reaction mixture was quenched with water (5 mL), extracted with DCM (2×10 mL), the organic layer was dried over MgSO4, filtered and evaporated the solvent to yield a pale white solid (0.335 g, 98%). ¹H NMR (400 MHz, DMSO) δ 8.22 (d, J=1.7 Hz, 1H), 8.17 (d, J=8.5 Hz, 1H), 8.02 (dd, J=8.5, 1.8 Hz, 1H), 7.9) (dd, J=6.2, 2.2 Hz, 1H), 7.73 (ddd, J=7.9, 5.3, 2.3 Hz, 1H), 7.48 (t, J=9.1 Hz, 1H), 4.38 (s, 2H).

To the crude were added water (2.5 mL). EtOH (2.5 mL) and DMF (0.25 mL). Refluxed the mixture and added Hydrazine hydrate (0.3 mL, 9.7 mmol) and left to reflux overnight. The reaction was cooled and the precipitate was collected by filtration, washed with EtOH (5 mL) and dried under high vacuum to afford compound 3B as a white solid (0.2 g, 60%) ¹H NMR (400 MHz, DMSO) δ 8.22 (d, J=1.7 Hz, 1H), 8.17 (d, J=8.5 Hz, 1H), 8.02 (dd, J=8.5, 1.8 Hz, 1H), 7.90 (dd, J=6.2, 2.2 Hz, 1H), 7.73 (ddd, J=7.9, 5.3, 2.3 Hz, 1H), 7.48 (t, J=9.1 Hz, 1H), 4.38 (s, 2H).

### Synthesis of Compound 4B

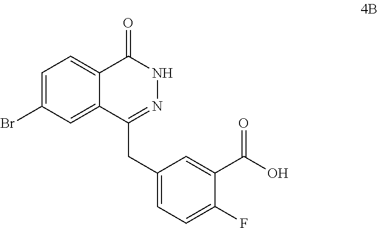 [SMILES: O=C(O)c1cc(Cc2n[nH]c(=O)c3ccc(Br)cc23)ccc1F]

Compound 3B (0.20 g, 0.57 mmol) and potassium hydroxide (0.323 g, 5.75 mmol) were added to ethanol (2 mL) and water (7 mL) and heated at 100° C. for 4.5 hours. The ethanol was evaporated off and the aqueous was extracted with ethyl acetate (2×5 mL). The aqueous was then acidified to pH=1 with conc. HCI to form a precipitate which was sonicated, filtered, washed with water and dried under high vacuum to afford a beige solid (0.2 g, 94% yield). ¹H NMR (400 MHz, DMSO) δ 12.68 (s, 1H), 8.27-8.07 (m, 2H), 7.99 (d, J=8.5 Hz, 1H), 7.86-7.71 (m, 1H), 7.62-7.48 (m, 1H), 7.24 (t, J=9.6 Hz, 1H), 4.35 (d, J=8.2 Hz, 2H).

### Synthesis of Compound 5B

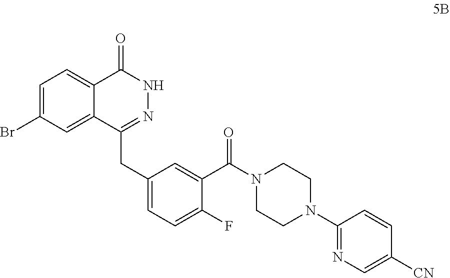 [SMILES: N#Cc1ccc(N2CCN(C(=O)c3cc(Cc4n[nH]c(=O)c5ccc(Br)cc45)ccc3F)CC2)nc1]

Compound 4B (100 mg, 0.266 mmol) and 6-(piperizino)pyridine-3-carbonitrile (50 mg, 0.266 mmol) were dissolved in 700 uL DMF in a small scintillation vile. 1-propane phosphoric acid cyclic anhydride (338 uL, 0.532 mmol) and DIPEA (140 μL, 0.798 mmol) were added via syringe and the reaction was stirred until complete conversion of the starting material was observed (2.5 hours). The reaction was monitored via TLC using 5% MeOH in DCM with 1-2 drops glacial acetic acid (to separate the starting material acid from product). The reaction was then dissolved in ethyl acetate (5 mL) and washed with saturated NaCO3, ddH2O, and brine. The organic layer was then concentrated to yield 102 mg of compound 5B (70% yield) ¹H NMR (400 MHz, DMSO) δ 12.69 (s, 1H), 8.50 (d, J=2.4 Hz, 1H), 8.16 (dd, J=5.2, 3.3 Hz, 2H), 7.94 (ddd, J=45.0, 8.8, 2.1 Hz, 2H), 7.41 (dd, J=8.5, 4.4 Hz, 2H), 7.26 (t, J=8.9 Hz, 1H), 6.91 (d, J=9.1 Hz, 1H), 4.34 (s, 2H), 3.74 (d, J=10.3 Hz, 4H), 3.61 (s, 2H).

### Synthesis of Example 10

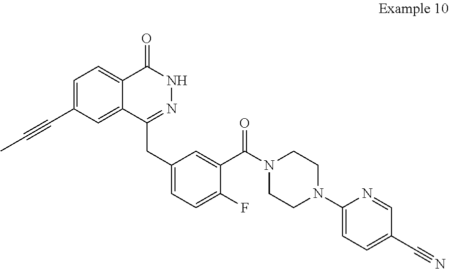 [SMILES: CC#Cc1ccc2c(=O)[nH]nc(Cc3ccc(F)c(C(=O)N4CCN(c5ccc(C#N)cn5)CC4)c3)c2c1]

In a clean, oven dried 15 mL round bottom flask. Compound 5B (50 mg, 0.092 mmol) was co-evaporated with dry toluene (5 mL) and allowed to sit under high vacuum for 20 mins. Dry toluene (1 mL) was added and argon was bubbled through the solution for 10 mins. Under argon pressure, Palladium Tetrakis (10.4 mg, 0.009 mmol) and Tributyl (1-propynyl) tin (30.4 uL, 0.1 mmol) were added and the reaction was refluxed under argon pressure for 3 hours until reaction had reached completion. The crude reaction was purified via dry loading method (1 g silica) on a 4 g silica manual column. The column was initially washed with 100% ethyl acetate to elute excess triphenyl phosphine, followed by 5% MeOH in ethyl acetate to separate impurities and elute desired compound. Fractions containing desired compound were pooled and concentrated to yield 22 mg of Example 10 (47% yield). 1H NMR (400 MHz, DMSO) δ 12.61 (s, 1H), 8.49 (s, 1H), 8.18 (d, J=8.2 Hz, 1H), 7.89 (d, J=18.6 Hz, 2H), 7.82-7.63 (m, 3H), 7.40 (s, 2H), 7.25 (d, J=8.5 Hz, 1H), 6.90 (d, J=9.2 Hz, 1H), 4.32 (s, 2H), 4.12 (s, 2H), 3.84-3.55 (m, 8H).

### EQUIVALENTS

While the present disclosure has been described in conjunction with the specific embodiments set forth above, many alternatives, modifications and other variations thereof will be apparent to those of ordinary skill in the art. All such alternatives, modifications and variations are intended to fall within the spirit and scope of the present disclosure.

1-3. (canceled)

4. A compound or a stereoisomer or a pharmaceutically acceptable salt thereof, wherein the compound is of Formula IV: 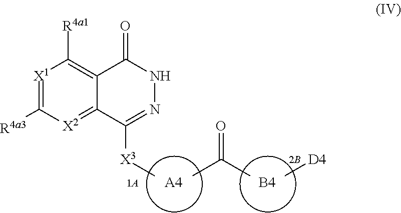
wherein:
X¹ is selected from the group of —N— and —CR^{4a2}—;
X² is selected from the group of —N— and —CR^{4a4}—;
R^{4a1}, R^{4a2}, R^{4a3}, and R^{4a4} are independently selected from the group of H, halo, —OH, C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, —SO₂-cycloalkyl, —NR^{4L1}R^{4L2}, and C₂₋₆alkynyl-NR^{4L3}R^{4L4};
wherein if X¹ is —CR^{4a2}— and X² is —CR^{4a4}—, then at least one of R^{4a1}, R^{4a2}, R^{4a3}, and R^{4a4} is not H;
wherein each C₁₋₆alkyl, C₂₋₆alkenyl, C₂₋₆alkynyl, cycloalkyl, heterocyclyl, aryl, heteroaryl, —O—C₁₋₆ alkyl, —O—C₂₋₆alkenyl, —O—C₂₋₆alkynyl, —O-cycloalkyl, —O-heterocyclyl, —O-aryl, —O-heteroaryl, or —SO₂-cycloalkyl of R^{4a1}, R^{4a2}, R^{4a3}, and R^{4a4} is unsubstituted or substituted with one or more substituents independently selected from the group consisting of halo, —OH, —CN, C₁₋₆alkyl, C₁₋₆haloalkyl, cycloalkyl, halocycloalkyl, —O—C₁₋₆alkyl, and —C(O)NH₂;
R^{4L1}, R^{4L2}, R^{4L3}, and R^{4L4} are independently selected from the group of H, C₁₋₆alkyl, C₂₋₆alkynyl, and cycloalkyl, wherein each C₁₋₆alkyl, C₂₋₆alkynyl, or cycloalkyl of R^{4L1}, R^{4L2}, R^{4L3}, and R^{4L4} is unsubstituted or substituted with one or more substituents independently selected from the group consisting of halo, C₁₋₆alkyl, C₁₋₆haloalkyl, cycloalkyl, and halocycloalkyl;
X³ is selected from the group of —O—, —NR^{4b1}—, and —CR^{4b2}R^{4b3}—, wherein R^{4b1}, R^{4b2}, and R^{4b3} are independently selected from the group of H and C₁₋₆alkyl;
A4 is selected from the group of 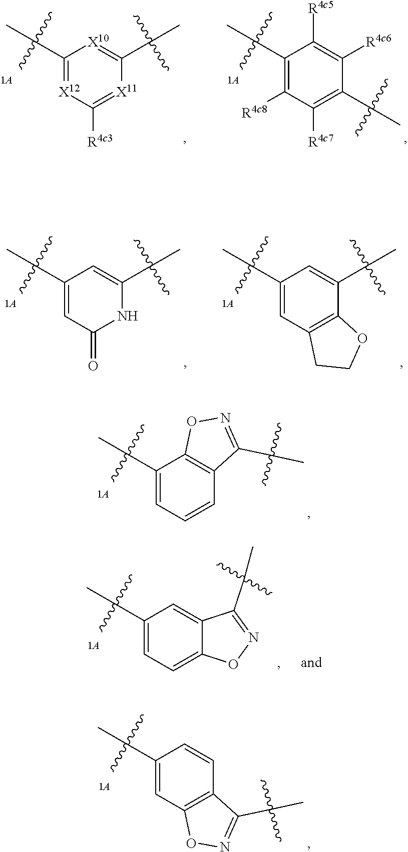
wherein a bond marked 1A is to X³;
X¹⁰ is selected from the group of —N— or —CR^{4c1}—, X¹¹ is —N— and —CR^{4c2}—, and X¹² is selected from the group of —N— and —CR^{4c4}—;
R^{4c1}, R^{4c2}, R^{4c3}, R^{4c4}, R^{4c5}, R^{4c6}, R^{4c7}, and R^{4c8} are independently selected from the group of H, halo, —CN, —SO₂CH₃, —SO₂NH₂, and —NHSO₂CH₃;
B4 is selected from the group of a 3 to 8-membered monocyclic heterocyclediyl, a 7 to 18-membered polycyclic heterocyclediyl, and a 7 to 18-membered spirocyclic heterocyclediyl;
wherein the 3 to 8-membered monocyclic heterocyclediyl, 7 to 18-membered polycyclic heterocyclediyl, or 7 to 18-membered spirocyclic heterocyclediyl of B4 is unsubstituted or substituted with one or more substituents selected from the group consisting of halo, C₁₋₆alkyl, and oxo;
provided that B4 is not 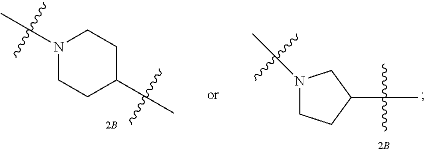 [SMILES: *C1CCN(*)CC1 ; *C1CCN(*)C1]
D4 is selected from the group of C₁₋₆alkyl, cycloalkyl, aryl, heteroaryl, —O—C₁₋₆alkyl, —O-aryl, —O-heteroaryl, —C(O)—C₁₋₆alkyl, —C(O)-cycloalkyl, —C(O)-heterocyclyl, —C(O)-aryl, —C(O)-heteroaryl, —N(R^{4D1})(R^{4D2}), —C(O)N(R^{4D3})(R^{4D4}), and —N(R^{4D5})C(O)R^{4D6};
wherein the C₁₋₆alkyl, cycloalkyl, aryl, heteroaryl, —O—C₁₋₆alkyl, —O-aryl, —O-heteroaryl, —C(O)—C₁₋₆ alkyl, —C(O)-cycloalkyl, —C(O)-heterocyclyl, —C(O)-aryl, or —C(O)-heteroaryl of D4 is unsubstituted or substituted with one or more substituents selected from the group consisting of halo, —CN, C₁₋₆alkyl, C₁₋₆haloalkyl, cycloalkyl, —OH, —O—C₁₋₆alkyl, C₁₋₆alkyl-OH, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{4D10})₂, wherein each R^{4D10} is independently selected from the group of H and C₁₋₆alkyl;
R^{4D1}, R^{4D3}, and R^{4D5} are independently selected from the group of H and C₁₋₆alkyl;
R^{4D2} is selected from the group of aryl and heteroaryl, wherein the aryl or heteroaryl of R^{4D2} is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆ alkyl-O—C₁₋₆haloalkyl, —C(O)-cycloalkyl, and —C(O)N(R^{4D11})₂, wherein each R^{4D11} is independently selected from the group of H and C₁₋₆alkyl; and
R^{4D4} and R^{4D6} are independently selected from the group of C₁₋₆alkyl, cycloalkyl, heterocyclyl, aryl, or heteroaryl, wherein each C₁₋₆alkyl, cycloalkyl, heterocyclyl, aryl, and heteroaryl of R^{4D4} and R^{4D6} is unsubstituted or substituted with one or more substituents independently selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, and —C(O)N(R^{4D12})₂, wherein each R^{4D12} is independently H or C₁₋₆alkyl;
provided that if B4 is 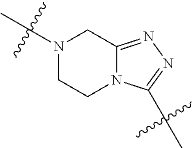 [SMILES: *c1nnc2n1CCN(*)C2]
and D4 is —CHF₂, —CF₂CH₃, or —CF₂CF₃, then R^{4a2} is not F, if X³ is —O—, B4 is 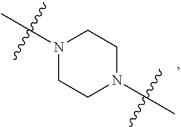
and D4 is —C(O)-aryl, then R^{4a4} is not Cl, and if D4 is 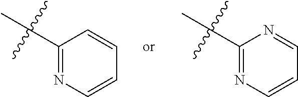 [SMILES: *c1ccccn1 ; *c1ncccn1]
and R^{4c2} is F, then R^{4a2} is not F.

5-7. (canceled)

8. The compound of claim 4, or a stereoisomer or a pharmaceutically acceptable salt thereof, wherein X¹ is —CR^{4a2}.

9. The compound of claim 4, or a stereoisomer or a pharmaceutically acceptable salt thereof, wherein X² is —CR^{4a4}.

10. The compound of claim 4, or a stereoisomer or a pharmaceutically acceptable salt thereof, wherein R^{4a2} is selected from the group of H, Cl, Br, I, —OH, C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, —SO₂-cycloalkyl, —NR^{1L1}R^{1L2}, C₂₋₆alkynyl-NR^{1L3}R^{1L4}, —NR^{2L1}R^{2L2}, C₂₋₆alkynyl-NR^{2L3}R^{2L4}, —NR^{4L1}R^{4L2}, and C₂₋₆alkynyl-NR^{4L3}R^{4L4};
wherein each C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, or —SO₂-cycloalkyl of R^{4a2} is unsubstituted or substituted with one or more substituents independently selected from the group consisting of halo, —OH, —CN, C₁₋₆alkyl, C₁₋₆haloalkyl, cycloalkyl, halocycloalkyl, —O—C₁₋₆alkyl, and —C(O)NH₂.

11. The compound of claim 4, or a stereoisomer or a pharmaceutically acceptable salt thereof, wherein R^{4a4} is selected from the group of H, F, Br, I, —OH, C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, —SO₂-cycloalkyl, —NR^{1L1}R^{1L2}, C₂₋₆alkynyl-NR^{1L3}R^{1L4}, —NR^{2L1}R^{2L2}, C₂₋₆alkynyl-NR^{2L3}R^{2L4}, —NR^{4L1}R^{4L2}, and C₂₋₆alkynyl-NR^{4L3}R^{4L4};
wherein each C₁₋₆alkyl, C₂₋₆alkenyl, C₂₋₆alkynyl, cycloalkyl, heterocyclyl, aryl, heteroaryl, —O—C₁₋₆alkyl, —O—C₂₋₆alkenyl, —O—C₂₋₆alkynyl, —O-cycloalkyl, —O-heterocyclyl, —O-aryl, —O-heteroaryl, or —SO₂-cycloalkyl of R^{4a4} is unsubstituted or substituted with one or more substituents independently selected from the group consisting of halo, —OH, —CN, C₁₋₆alkyl, C₁₋₆haloalkyl, cycloalkyl, halocycloalkyl, —O—C₁₋₆alkyl, and —C(O)NH₂.

12. (canceled)

13. The compound of claim 4, or a stereoisomer or a pharmaceutically acceptable salt thereof, wherein R^{4a3} is not H.

14. (canceled)

15. The compound of claim 4, or a stereoisomer or a pharmaceutically acceptable salt thereof, wherein R^{4a1} is selected from the group of F and Cl.

16. (canceled)

17. The compound of claim 4, or a stereoisomer or a pharmaceutically acceptable salt thereof, wherein R^{4a3} is selected from the group of F and Cl.

18-20. (canceled)

21. The compound of claim 4, or a stereoisomer or a pharmaceutically acceptable salt thereof, wherein R^{4a3} is selected from the group of —O—C₁₋₆alkyl and —O-cycloalkyl, wherein the —O—C₁₋₆alkyl or —O-cycloalkyl is unsubstituted or substituted with halo or —CN.

22. (canceled)

23. The compound of claim 4, or a stereoisomer or a pharmaceutically acceptable salt thereof, wherein R^{4a3} is —O—C₂₋₆alkynyl, wherein the —O—C₂₋₆alkynyl is unsubstituted or substituted with halo.

24. (canceled)

25. The compound of claim 4, or a stereoisomer or a pharmaceutically acceptable salt thereof, wherein R^{4a3} is selected from the group of —O-aryl and —O-heteroaryl, wherein the —O-aryl or —O-heteroaryl is unsubstituted or substituted with halo.

26-27. (canceled)

28. The compound of claim 4, or a stereoisomer or a pharmaceutically acceptable salt thereof, wherein R^{4a2} is selected from the group of F and Cl and R^{4a3} is selected from the group of —O—C₁₋₆alkyl and —O-cycloalkyl, wherein the —O—C₁₋₆alkyl or —O-cycloalkyl is unsubstituted or substituted with halo.

29. (canceled)

30. The compound of claim 4, or a stereoisomer or a pharmaceutically acceptable salt thereof, wherein X³ is —CH₂—.

31-33. (canceled)

34. The compound of claim 4, or a stereoisomer or a pharmaceutically acceptable salt thereof, wherein A4 is selected from the group of 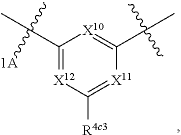
wherein X¹⁰ is —CR^{4c1}—, X¹¹ is —CR^{4c2}—, and X¹² is —CR^{4c3}—.

35-44. (canceled)

46. The compound of claim 4, or a stereoisomer or a pharmaceutically acceptable salt thereof, wherein B4 is

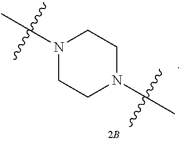

47-48. (canceled)

49. The compound of claim 4, or a stereoisomer or a pharmaceutically acceptable salt thereof, wherein B4 is

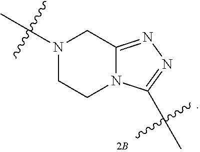 [SMILES: *c1nnc2n1CCN(*)C2]

50-63. (canceled)

64. The compound of claim 4, or a stereoisomer or a pharmaceutically acceptable salt thereof, wherein D4 is a monocyclic 6-membered aryl, wherein the monocyclic 6-membered aryl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆alkyl, C₁₋₆alkyl-O—C₁₋₆haloalkyl, —C(O)-cycloalkyl, —C(O)NH₂, —C(O)NHCH₃, and —C(O)N(CH₃)₂.

65. (canceled)

66. The compound of claim 4, or a stereoisomer or a pharmaceutically acceptable salt thereof, wherein D4 is a monocyclic 5 or 6-membered heteroaryl comprising one or more N, wherein the monocyclic 5 or 6-membered heteroaryl is unsubstituted or substituted with one or more substituents selected from the group consisting of halo, —CN, C₁₋₆alkyl, C₁₋₆haloalkyl, cycloalkyl, —OH, —O—C₁₋₆alkyl, —O—C₁₋₆haloalkyl, C₁₋₆alkyl-O—C₁₋₆-alkyl, C₁₋₆alkyl-O—C₁₋₆haloalkyl, —C(O)-cycloalkyl, —C(O)NH₂, —C(O)NHCH₃, and —C(O)N(CH₃)₂.

67-74. (canceled)

75. The compound of claim 4, or a stereoisomer or a pharmaceutically acceptable salt thereof, wherein the compound is selected from the group consisting of

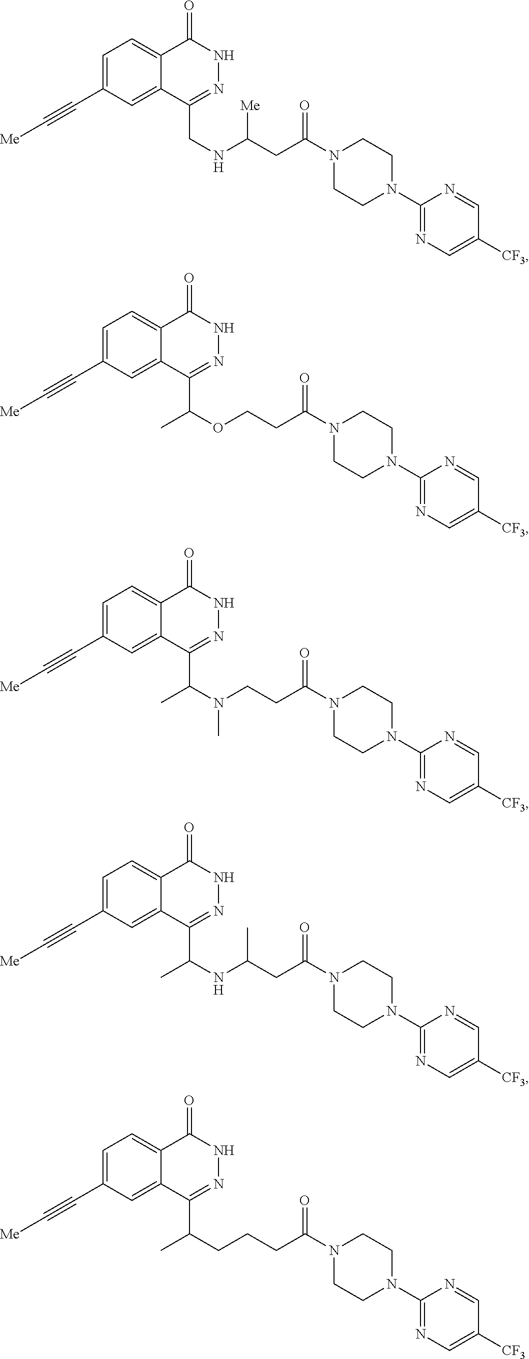 [SMILES: CC#Cc1ccc2c(=O)[nH]nc(CNC(C)CC(=O)N3CCN(c4ncc(C(F)(F)F)cn4)CC3)c2c1 ; CC#Cc1ccc2c(=O)[nH]nc(C(C)OCCC(=O)N3CCN(c4ncc(C(F)(F)F)cn4)CC3)c2c1 ; CC#Cc1ccc2c(=O)[nH]nc(C(C)N(C)CCC(=O)N3CCN(c4ncc(C(F)(F)F)cn4)CC3)c2c1 ; CC#Cc1ccc2c(=O)[nH]nc(C(C)NC(C)CC(=O)N3CCN(c4ncc(C(F)(F)F)cn4)CC3)c2c1 ; CC#Cc1ccc2c(=O)[nH]nc(C(C)CCCC(=O)N3CCN(c4ncc(C(F)(F)F)cn4)CC3)c2c1]

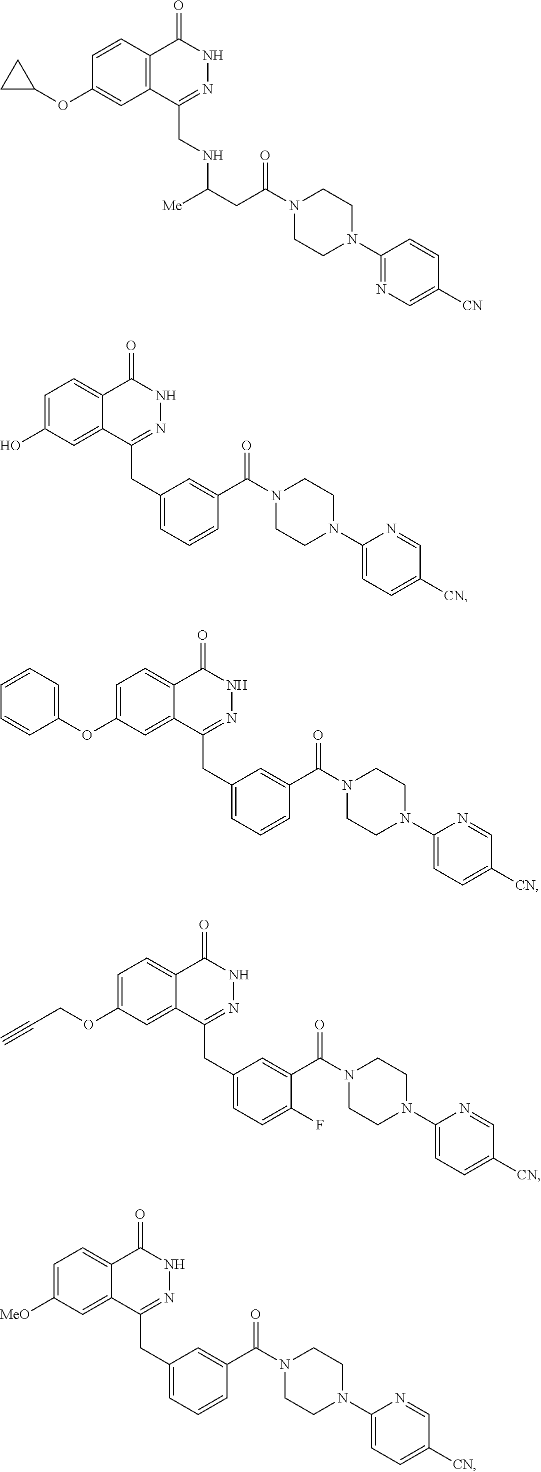 [SMILES: CC(CC(=O)N1CCN(c2ccc(C#N)cn2)CC1)NCc1n[nH]c(=O)c2ccc(OC3CC3)cc12 ; N#Cc1ccc(N2CCN(C(=O)c3cccc(Cc4n[nH]c(=O)c5ccc(O)cc45)c3)CC2)nc1 ; N#Cc1ccc(N2CCN(C(=O)c3cccc(Cc4n[nH]c(=O)c5ccc(Oc6ccccc6)cc45)c3)CC2)nc1 ; C#CCOc1ccc2c(=O)[nH]nc(Cc3ccc(F)c(C(=O)N4CCN(c5ccc(C#N)cn5)CC4)c3)c2c1 ; COc1ccc2c(=O)[nH]nc(Cc3cccc(C(=O)N4CCN(c5ccc(C#N)cn5)CC4)c3)c2c1]

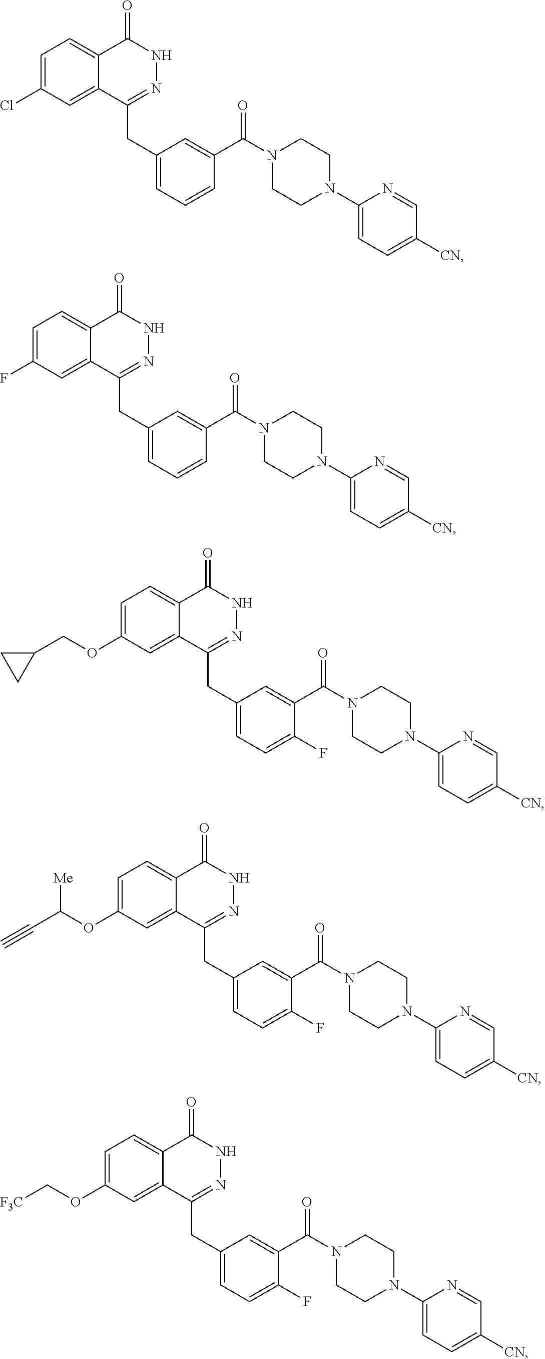 [SMILES: N#Cc1ccc(N2CCN(C(=O)c3cccc(Cc4n[nH]c(=O)c5ccc(Cl)cc45)c3)CC2)nc1 ; N#Cc1ccc(N2CCN(C(=O)c3cccc(Cc4n[nH]c(=O)c5ccc(F)cc45)c3)CC2)nc1 ; N#Cc1ccc(N2CCN(C(=O)c3cc(Cc4n[nH]c(=O)c5ccc(OCC6CC6)cc45)ccc3F)CC2)nc1 ; C#CC(C)Oc1ccc2c(=O)[nH]nc(Cc3ccc(F)c(C(=O)N4CCN(c5ccc(C#N)cn5)CC4)c3)c2c1 ; N#Cc1ccc(N2CCN(C(=O)c3cc(Cc4n[nH]c(=O)c5ccc(OCC(F)(F)F)cc45)ccc3F)CC2)nc1]

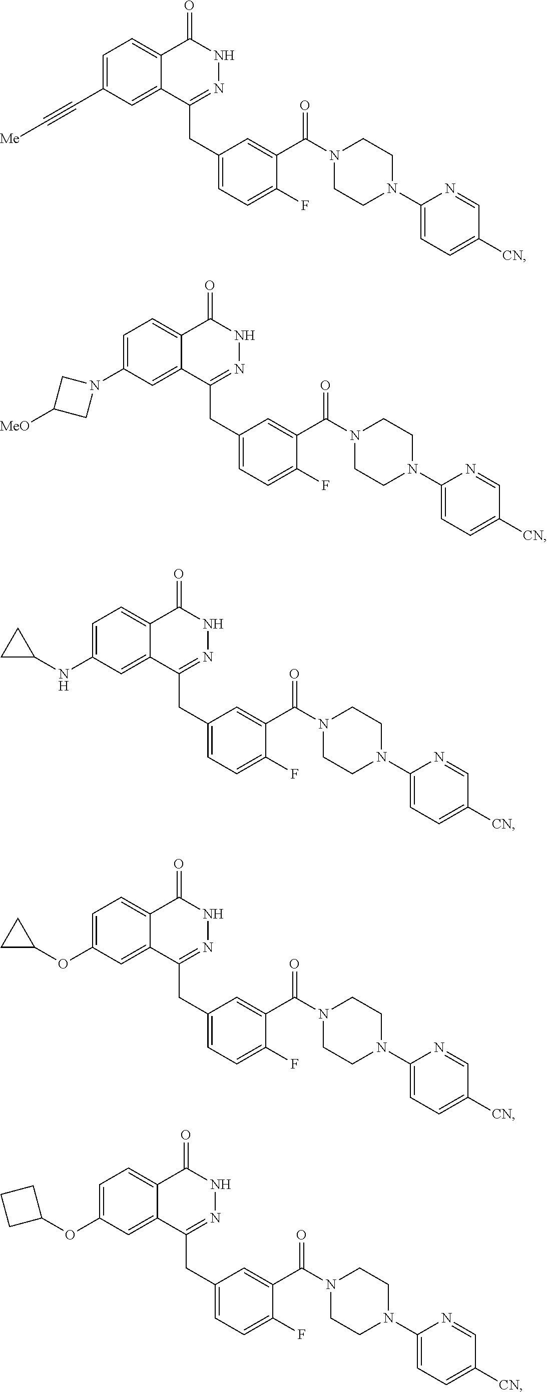 [SMILES: CC#Cc1ccc2c(=O)[nH]nc(Cc3ccc(F)c(C(=O)N4CCN(c5ccc(C#N)cn5)CC4)c3)c2c1 ; COC1CN(c2ccc3c(=O)[nH]nc(Cc4ccc(F)c(C(=O)N5CCN(c6ccc(C#N)cn6)CC5)c4)c3c2)C1 ; N#Cc1ccc(N2CCN(C(=O)c3cc(Cc4n[nH]c(=O)c5ccc(NC6CC6)cc45)ccc3F)CC2)nc1 ; N#Cc1ccc(N2CCN(C(=O)c3cc(Cc4n[nH]c(=O)c5ccc(OC6CC6)cc45)ccc3F)CC2)nc1 ; N#Cc1ccc(N2CCN(C(=O)c3cc(Cc4n[nH]c(=O)c5ccc(OC6CCC6)cc45)ccc3F)CC2)nc1]

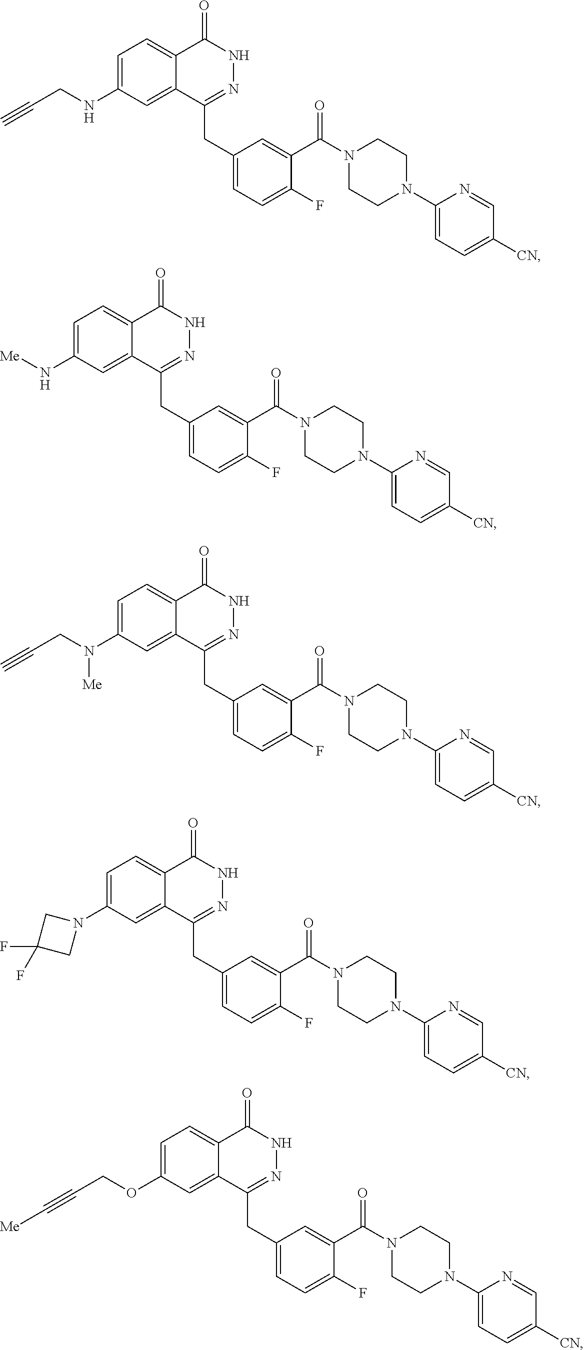 [SMILES: C#CCNc1ccc2c(=O)[nH]nc(Cc3ccc(F)c(C(=O)N4CCN(c5ccc(C#N)cn5)CC4)c3)c2c1 ; CNc1ccc2c(=O)[nH]nc(Cc3ccc(F)c(C(=O)N4CCN(c5ccc(C#N)cn5)CC4)c3)c2c1 ; C#CCN(C)c1ccc2c(=O)[nH]nc(Cc3ccc(F)c(C(=O)N4CCN(c5ccc(C#N)cn5)CC4)c3)c2c1 ; N#Cc1ccc(N2CCN(C(=O)c3cc(Cc4n[nH]c(=O)c5ccc(N6CC(F)(F)C6)cc45)ccc3F)CC2)nc1 ; CC#CCOc1ccc2c(=O)[nH]nc(Cc3ccc(F)c(C(=O)N4CCN(c5ccc(C#N)cn5)CC4)c3)c2c1]

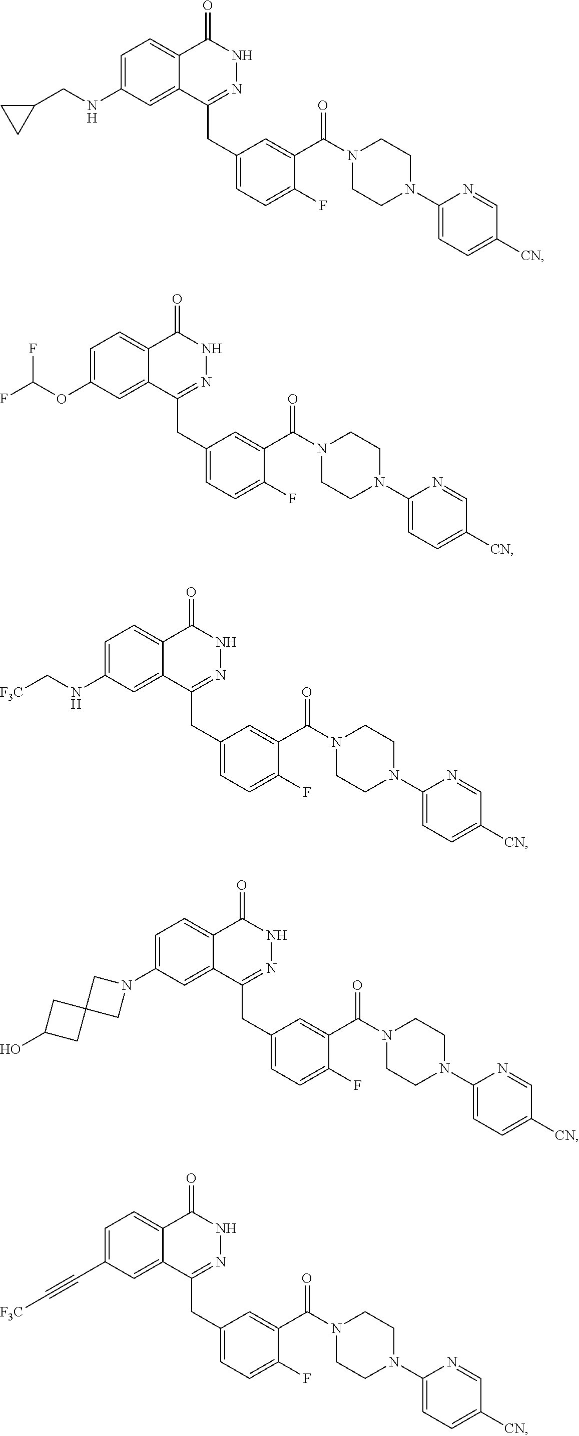 [SMILES: N#Cc1ccc(N2CCN(C(=O)c3cc(Cc4n[nH]c(=O)c5ccc(NCC6CC6)cc45)ccc3F)CC2)nc1 ; N#Cc1ccc(N2CCN(C(=O)c3cc(Cc4n[nH]c(=O)c5ccc(OC(F)F)cc45)ccc3F)CC2)nc1 ; N#Cc1ccc(N2CCN(C(=O)c3cc(Cc4n[nH]c(=O)c5ccc(NCC(F)(F)F)cc45)ccc3F)CC2)nc1 ; N#Cc1ccc(N2CCN(C(=O)c3cc(Cc4n[nH]c(=O)c5ccc(N6CC7(CC(O)C7)C6)cc45)ccc3F)CC2)nc1 ; N#Cc1ccc(N2CCN(C(=O)c3cc(Cc4n[nH]c(=O)c5ccc(C#CC(F)(F)F)cc45)ccc3F)CC2)nc1]

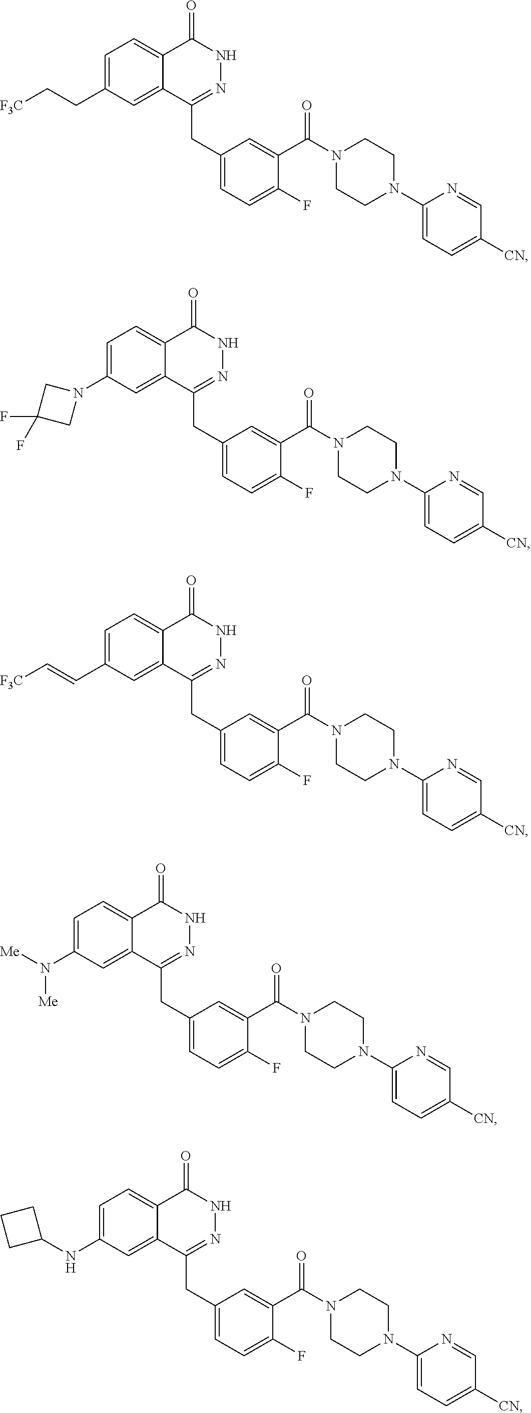 [SMILES: N#Cc1ccc(N2CCN(C(=O)c3cc(Cc4n[nH]c(=O)c5ccc(CCC(F)(F)F)cc45)ccc3F)CC2)nc1 ; N#Cc1ccc(N2CCN(C(=O)c3cc(Cc4n[nH]c(=O)c5ccc(N6CC(F)(F)C6)cc45)ccc3F)CC2)nc1 ; N#Cc1ccc(N2CCN(C(=O)c3cc(Cc4n[nH]c(=O)c5ccc(/C=C/C(F)(F)F)cc45)ccc3F)CC2)nc1 ; CN(C)c1ccc2c(=O)[nH]nc(Cc3ccc(F)c(C(=O)N4CCN(c5ccc(C#N)cn5)CC4)c3)c2c1 ; N#Cc1ccc(N2CCN(C(=O)c3cc(Cc4n[nH]c(=O)c5ccc(NC6CCC6)cc45)ccc3F)CC2)nc1]

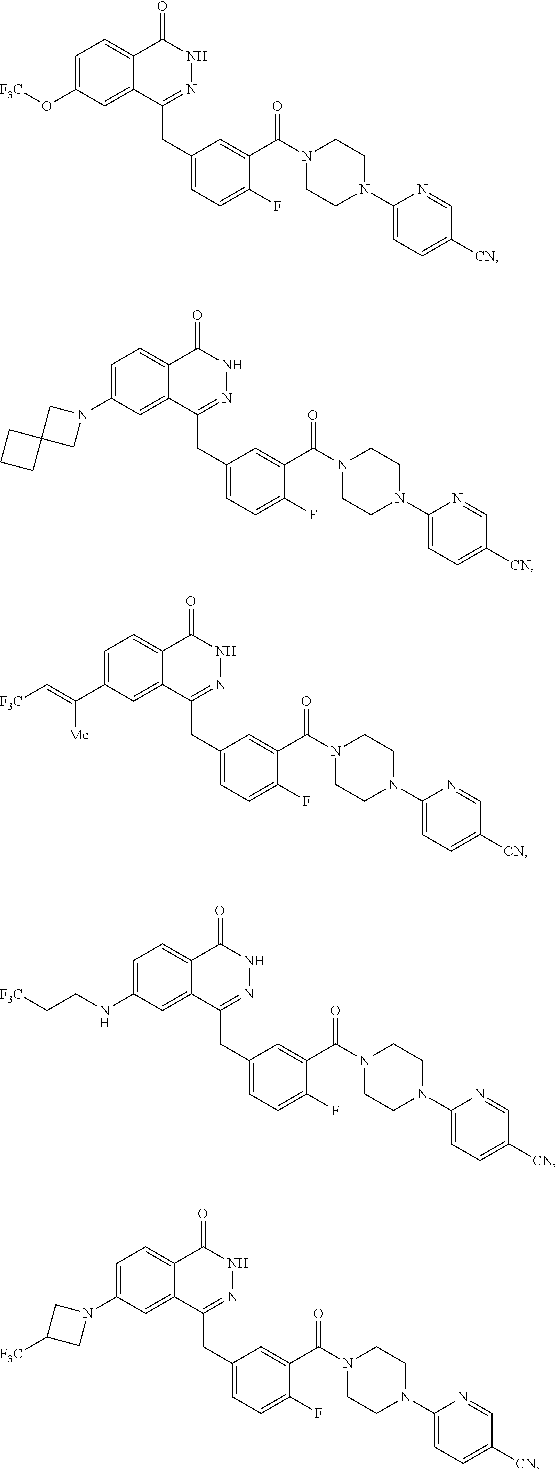 [SMILES: N#Cc1ccc(N2CCN(C(=O)c3cc(Cc4n[nH]c(=O)c5ccc(OC(F)(F)F)cc45)ccc3F)CC2)nc1 ; N#Cc1ccc(N2CCN(C(=O)c3cc(Cc4n[nH]c(=O)c5ccc(N6CC7(CCC7)C6)cc45)ccc3F)CC2)nc1 ; C/C(=C\C(F)(F)F)c1ccc2c(=O)[nH]nc(Cc3ccc(F)c(C(=O)N4CCN(c5ccc(C#N)cn5)CC4)c3)c2c1 ; N#Cc1ccc(N2CCN(C(=O)c3cc(Cc4n[nH]c(=O)c5ccc(NCCC(F)(F)F)cc45)ccc3F)CC2)nc1 ; N#Cc1ccc(N2CCN(C(=O)c3cc(Cc4n[nH]c(=O)c5ccc(N6CC(C(F)(F)F)C6)cc45)ccc3F)CC2)nc1]

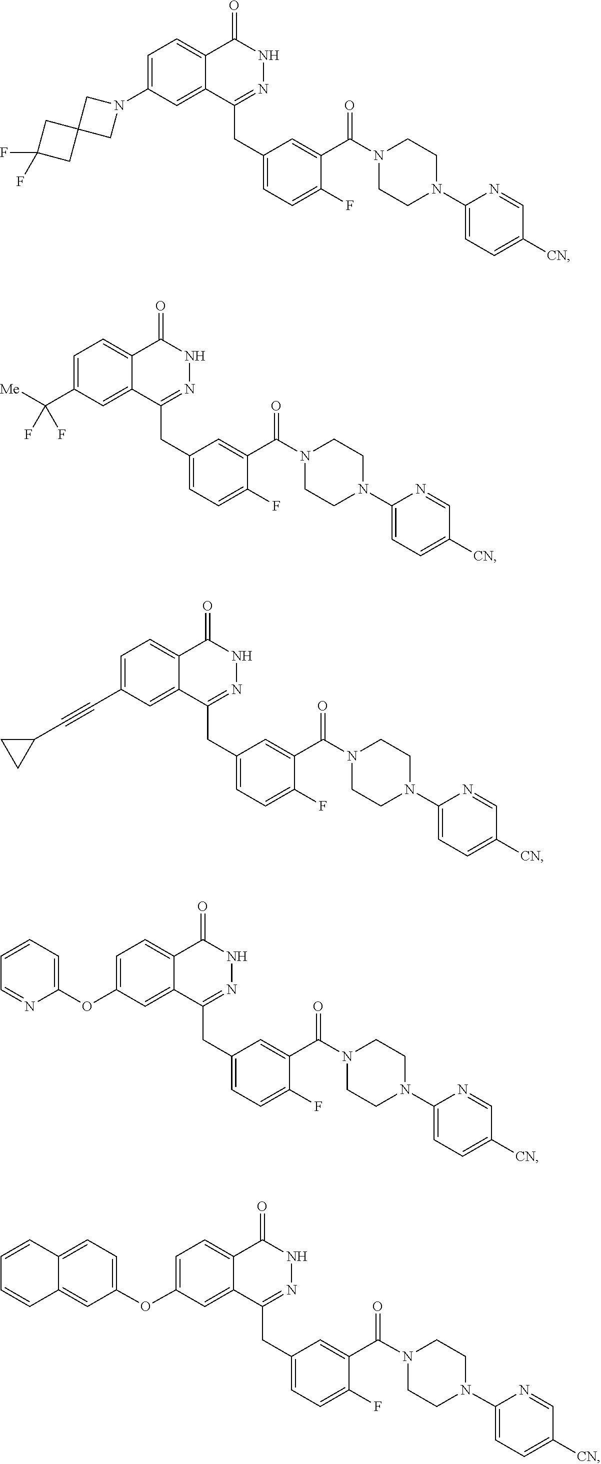 [SMILES: N#Cc1ccc(N2CCN(C(=O)c3cc(Cc4n[nH]c(=O)c5ccc(N6CC7(C6)CC(F)(F)C7)cc45)ccc3F)CC2)nc1 ; CC(F)(F)c1ccc2c(=O)[nH]nc(Cc3ccc(F)c(C(=O)N4CCN(c5ccc(C#N)cn5)CC4)c3)c2c1 ; N#Cc1ccc(N2CCN(C(=O)c3cc(Cc4n[nH]c(=O)c5ccc(C#CC6CC6)cc45)ccc3F)CC2)nc1 ; N#Cc1ccc(N2CCN(C(=O)c3cc(Cc4n[nH]c(=O)c5ccc(Oc6ccccn6)cc45)ccc3F)CC2)nc1 ; N#Cc1ccc(N2CCN(C(=O)c3cc(Cc4n[nH]c(=O)c5ccc(Oc6ccc7ccccc7c6)cc45)ccc3F)CC2)nc1]

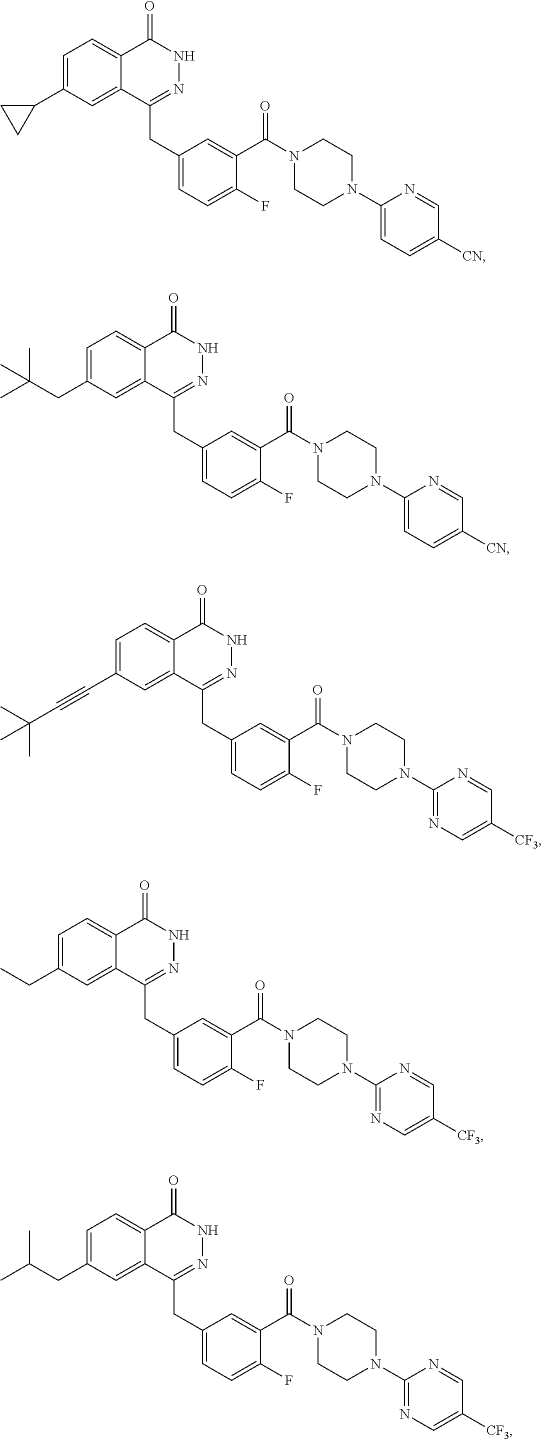 [SMILES: N#Cc1ccc(N2CCN(C(=O)c3cc(Cc4n[nH]c(=O)c5ccc(C6CC6)cc45)ccc3F)CC2)nc1 ; CC(C)(C)Cc1ccc2c(=O)[nH]nc(Cc3ccc(F)c(C(=O)N4CCN(c5ccc(C#N)cn5)CC4)c3)c2c1 ; CC(C)(C)C#Cc1ccc2c(=O)[nH]nc(Cc3ccc(F)c(C(=O)N4CCN(c5ncc(C(F)(F)F)cn5)CC4)c3)c2c1 ; CCc1ccc2c(=O)[nH]nc(Cc3ccc(F)c(C(=O)N4CCN(c5ncc(C(F)(F)F)cn5)CC4)c3)c2c1 ; CC(C)Cc1ccc2c(=O)[nH]nc(Cc3ccc(F)c(C(=O)N4CCN(c5ncc(C(F)(F)F)cn5)CC4)c3)c2c1]

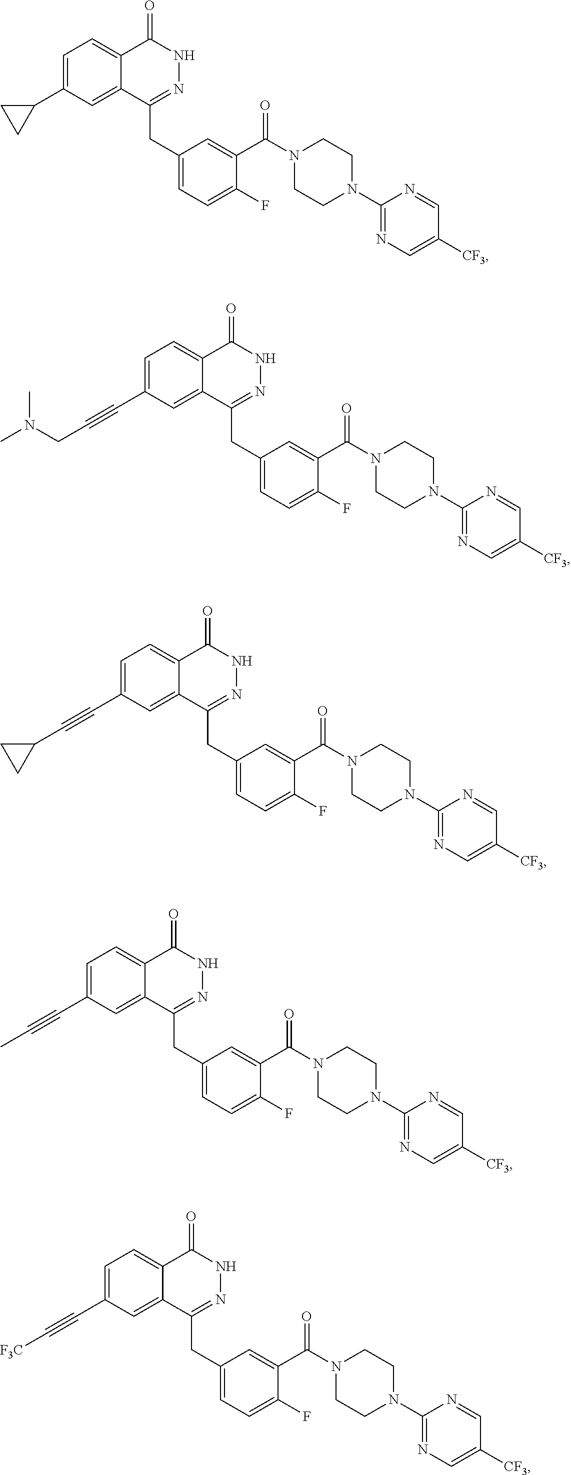 [SMILES: O=C(c1cc(Cc2n[nH]c(=O)c3ccc(C4CC4)cc23)ccc1F)N1CCN(c2ncc(C(F)(F)F)cn2)CC1 ; CN(C)CC#Cc1ccc2c(=O)[nH]nc(Cc3ccc(F)c(C(=O)N4CCN(c5ncc(C(F)(F)F)cn5)CC4)c3)c2c1 ; O=C(c1cc(Cc2n[nH]c(=O)c3ccc(C#CC4CC4)cc23)ccc1F)N1CCN(c2ncc(C(F)(F)F)cn2)CC1 ; CC#Cc1ccc2c(=O)[nH]nc(Cc3ccc(F)c(C(=O)N4CCN(c5ncc(C(F)(F)F)cn5)CC4)c3)c2c1 ; O=C(c1cc(Cc2n[nH]c(=O)c3ccc(C#CC(F)(F)F)cc23)ccc1F)N1CCN(c2ncc(C(F)(F)F)cn2)CC1]

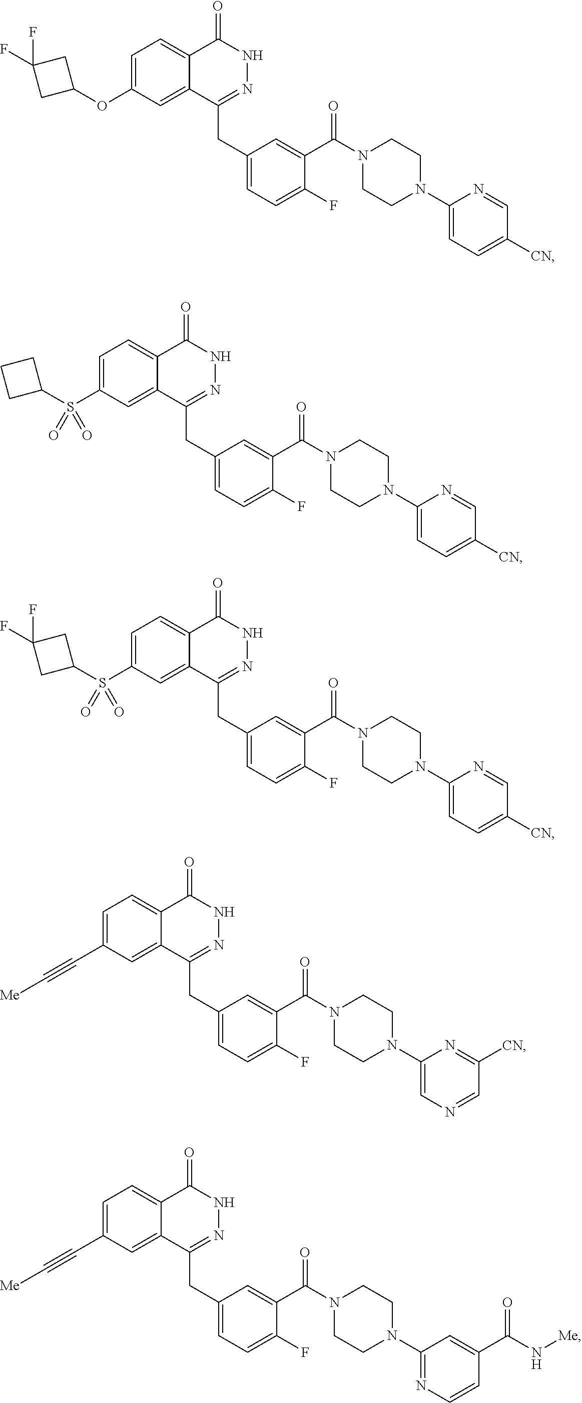 [SMILES: N#Cc1ccc(N2CCN(C(=O)c3cc(Cc4n[nH]c(=O)c5ccc(OC6CC(F)(F)C6)cc45)ccc3F)CC2)nc1 ; N#Cc1ccc(N2CCN(C(=O)c3cc(Cc4n[nH]c(=O)c5ccc(S(=O)(=O)C6CCC6)cc45)ccc3F)CC2)nc1 ; N#Cc1ccc(N2CCN(C(=O)c3cc(Cc4n[nH]c(=O)c5ccc(S(=O)(=O)C6CC(F)(F)C6)cc45)ccc3F)CC2)nc1 ; CC#Cc1ccc2c(=O)[nH]nc(Cc3ccc(F)c(C(=O)N4CCN(c5cncc(C#N)n5)CC4)c3)c2c1 ; CC#Cc1ccc2c(=O)[nH]nc(Cc3ccc(F)c(C(=O)N4CCN(c5cc(C(=O)NC)ccn5)CC4)c3)c2c1]

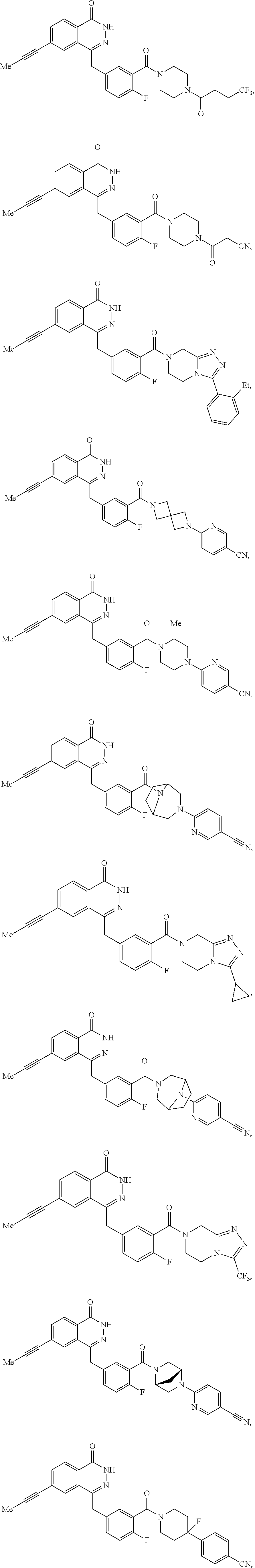

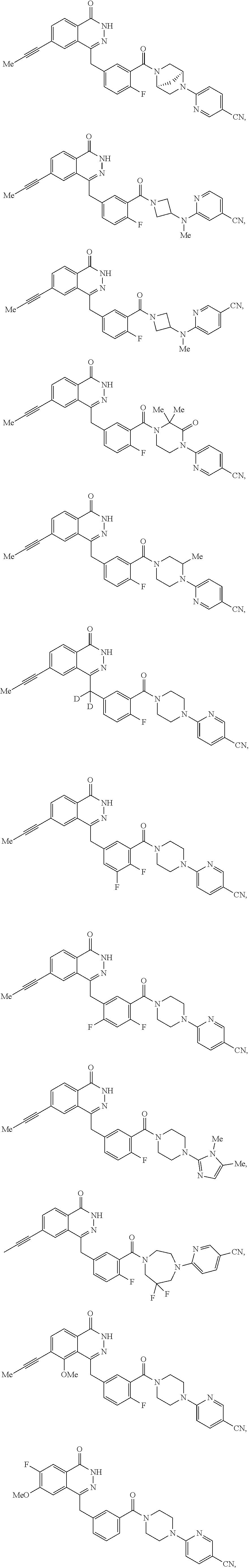

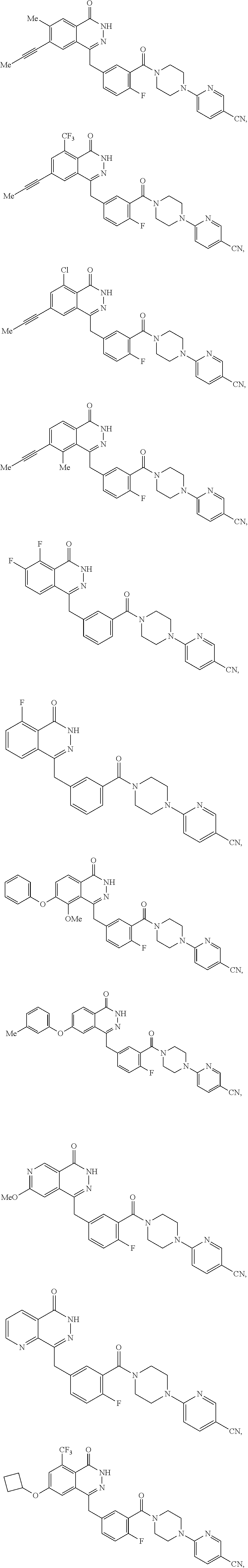

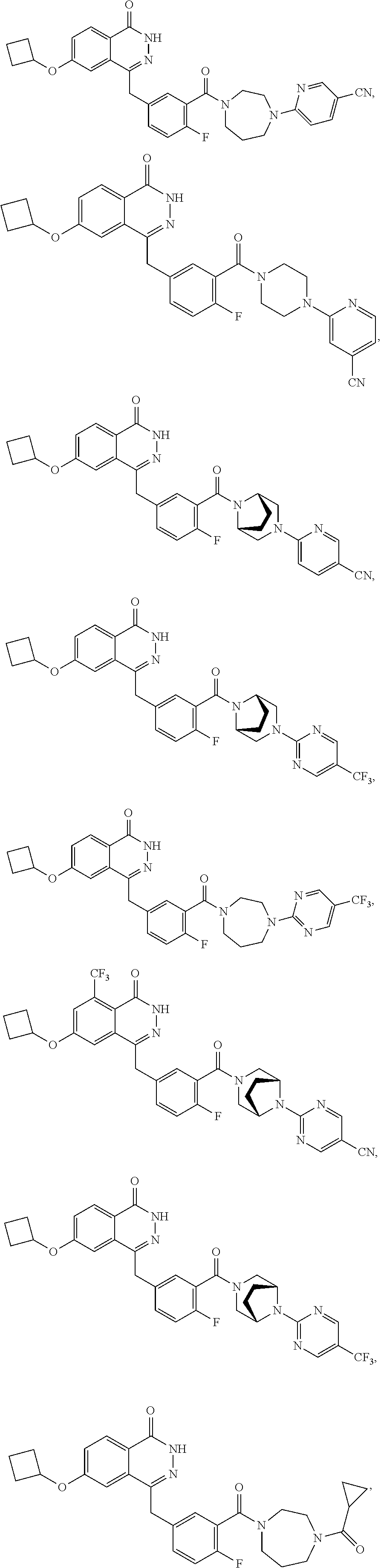

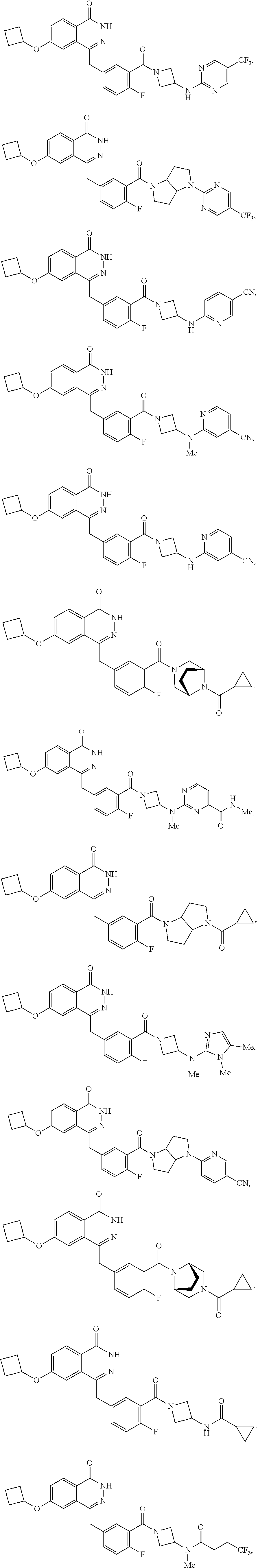

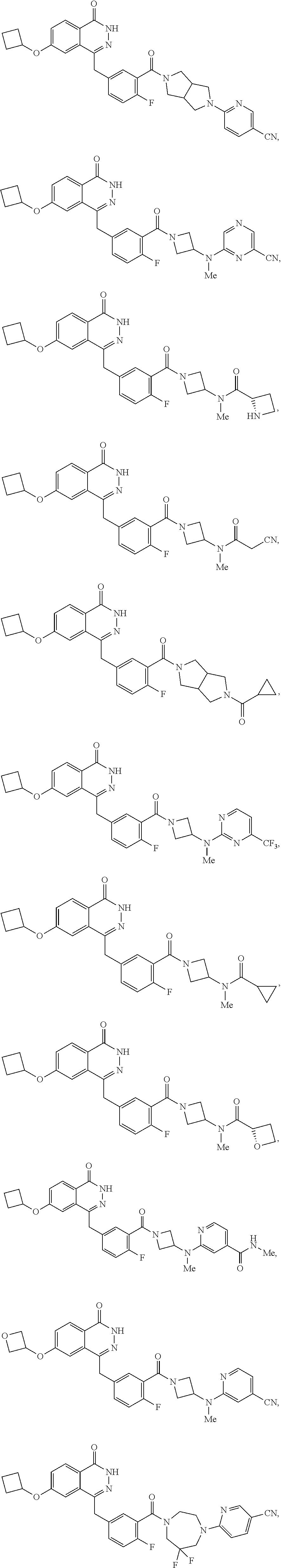

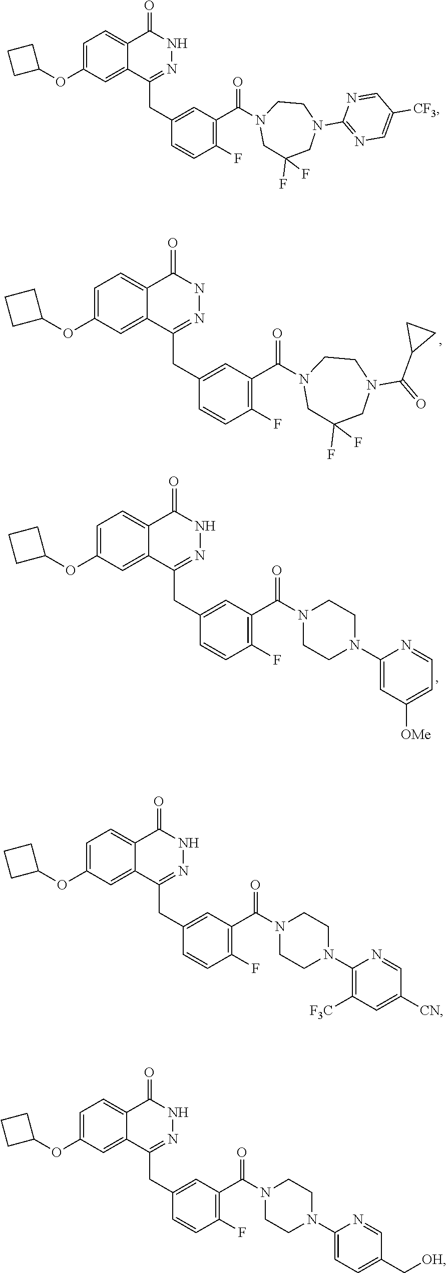 [SMILES: O=C(c1cc(Cc2n[nH]c(=O)c3ccc(OC4CCC4)cc23)ccc1F)N1CCN(c2ncc(C(F)(F)F)cn2)CC(F)(F)C1 ; O=C(c1cc(Cc2n[nH]c(=O)c3ccc(OC4CCC4)cc23)ccc1F)N1CCN(C(=O)C2CC2)CC(F)(F)C1 ; COc1ccnc(N2CCN(C(=O)c3cc(Cc4n[nH]c(=O)c5ccc(OC6CCC6)cc45)ccc3F)CC2)c1 ; N#Cc1cnc(N2CCN(C(=O)c3cc(Cc4n[nH]c(=O)c5ccc(OC6CCC6)cc45)ccc3F)CC2)c(C(F)(F)F)c1 ; O=C(c1cc(Cc2n[nH]c(=O)c3ccc(OC4CCC4)cc23)ccc1F)N1CCN(c2ccc(CO)cn2)CC1]

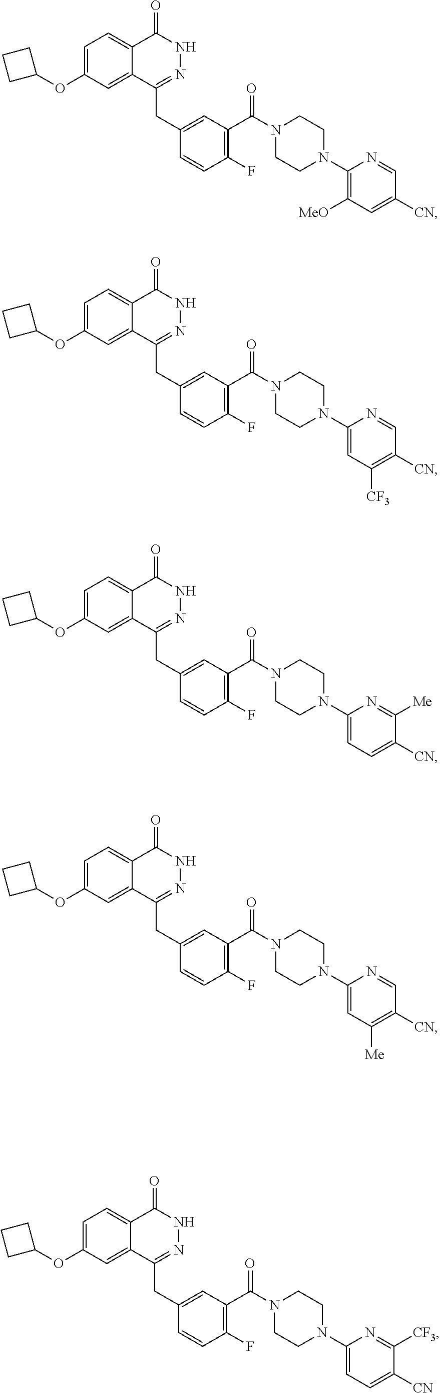 [SMILES: COc1cc(C#N)cnc1N1CCN(C(=O)c2cc(Cc3n[nH]c(=O)c4ccc(OC5CCC5)cc34)ccc2F)CC1 ; N#Cc1cnc(N2CCN(C(=O)c3cc(Cc4n[nH]c(=O)c5ccc(OC6CCC6)cc45)ccc3F)CC2)cc1C(F)(F)F ; Cc1nc(N2CCN(C(=O)c3cc(Cc4n[nH]c(=O)c5ccc(OC6CCC6)cc45)ccc3F)CC2)ccc1C#N ; Cc1cc(N2CCN(C(=O)c3cc(Cc4n[nH]c(=O)c5ccc(OC6CCC6)cc45)ccc3F)CC2)ncc1C#N ; N#Cc1ccc(N2CCN(C(=O)c3cc(Cc4n[nH]c(=O)c5ccc(OC6CCC6)cc45)ccc3F)CC2)nc1C(F)(F)F]

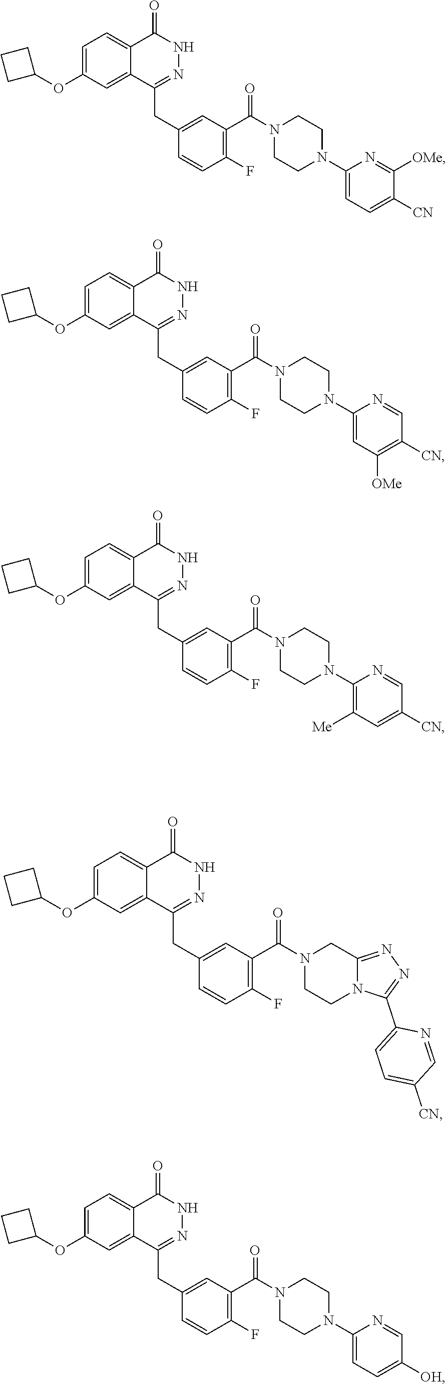 [SMILES: COc1nc(N2CCN(C(=O)c3cc(Cc4n[nH]c(=O)c5ccc(OC6CCC6)cc45)ccc3F)CC2)ccc1C#N ; COc1cc(N2CCN(C(=O)c3cc(Cc4n[nH]c(=O)c5ccc(OC6CCC6)cc45)ccc3F)CC2)ncc1C#N ; Cc1cc(C#N)cnc1N1CCN(C(=O)c2cc(Cc3n[nH]c(=O)c4ccc(OC5CCC5)cc34)ccc2F)CC1 ; N#Cc1ccc(-c2nnc3n2CCN(C(=O)c2cc(Cc4n[nH]c(=O)c5ccc(OC6CCC6)cc45)ccc2F)C3)nc1 ; O=C(c1cc(Cc2n[nH]c(=O)c3ccc(OC4CCC4)cc23)ccc1F)N1CCN(c2ccc(O)cn2)CC1]

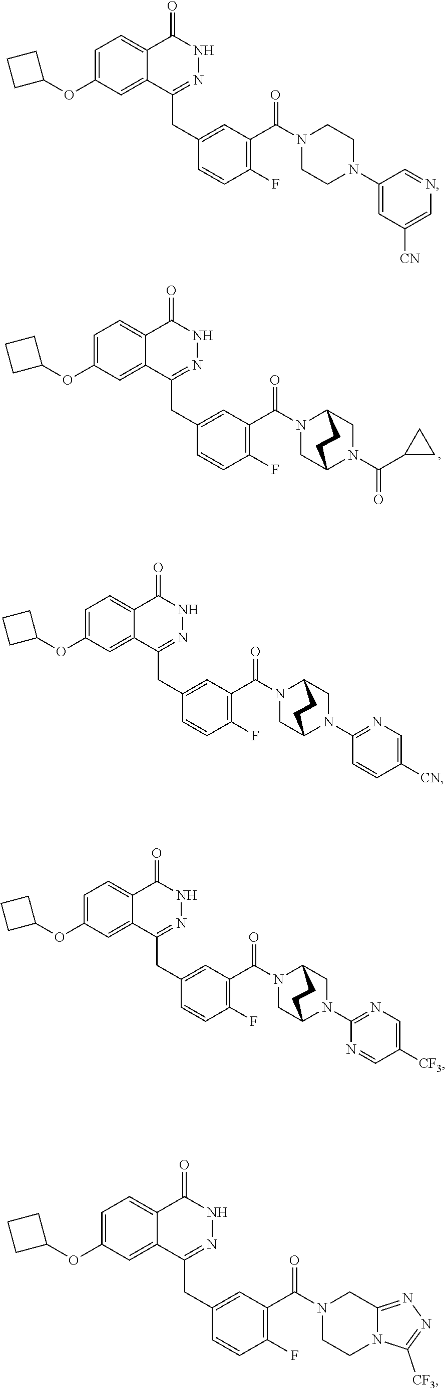

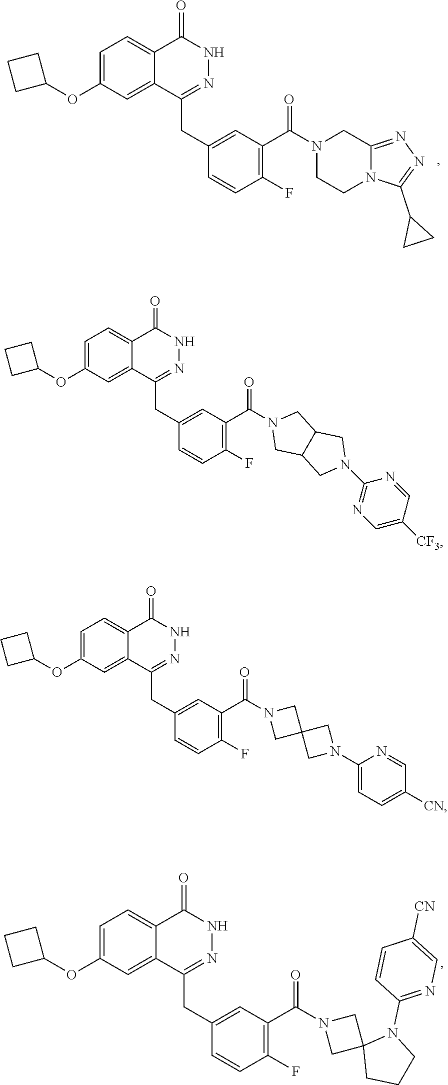 [SMILES: O=C(c1cc(Cc2n[nH]c(=O)c3ccc(OC4CCC4)cc23)ccc1F)N1CCn2c(nnc2C2CC2)C1 ; O=C(c1cc(Cc2n[nH]c(=O)c3ccc(OC4CCC4)cc23)ccc1F)N1CC2CN(c3ncc(C(F)(F)F)cn3)CC2C1 ; N#Cc1ccc(N2CC3(CN(C(=O)c4cc(Cc5n[nH]c(=O)c6ccc(OC7CCC7)cc56)ccc4F)C3)C2)nc1 ; N#Cc1ccc(N2CCCC23CN(C(=O)c2cc(Cc4n[nH]c(=O)c5ccc(OC6CCC6)cc45)ccc2F)C3)nc1]

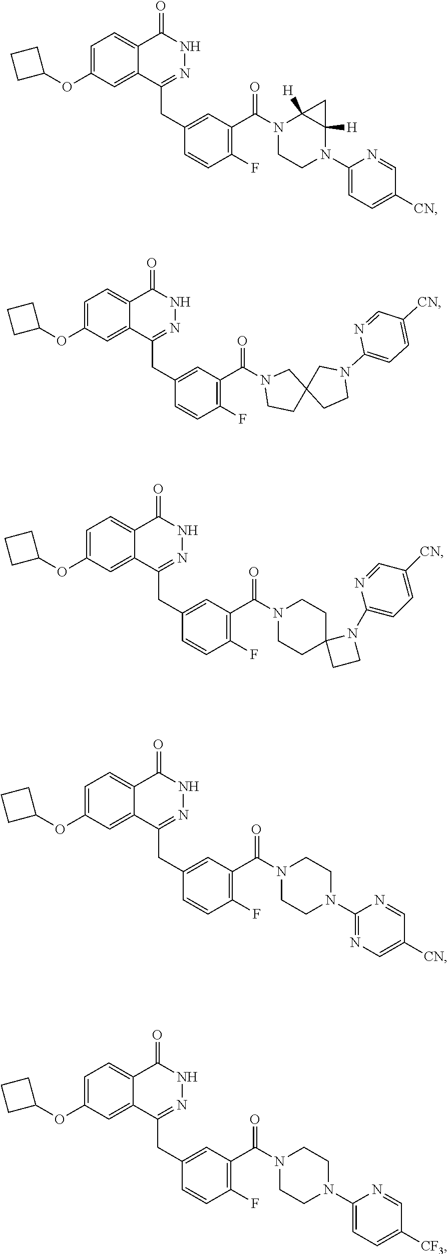 [SMILES: N#Cc1ccc(N2CCN(C(=O)c3cc(Cc4n[nH]c(=O)c5ccc(OC6CCC6)cc45)ccc3F)[C@@H]3C[C@@H]32)nc1 ; N#Cc1ccc(N2CCC3(CCN(C(=O)c4cc(Cc5n[nH]c(=O)c6ccc(OC7CCC7)cc56)ccc4F)C3)C2)nc1 ; N#Cc1ccc(N2CCC23CCN(C(=O)c2cc(Cc4n[nH]c(=O)c5ccc(OC6CCC6)cc45)ccc2F)CC3)nc1 ; N#Cc1cnc(N2CCN(C(=O)c3cc(Cc4n[nH]c(=O)c5ccc(OC6CCC6)cc45)ccc3F)CC2)nc1 ; O=C(c1cc(Cc2n[nH]c(=O)c3ccc(OC4CCC4)cc23)ccc1F)N1CCN(c2ccc(C(F)(F)F)cn2)CC1]

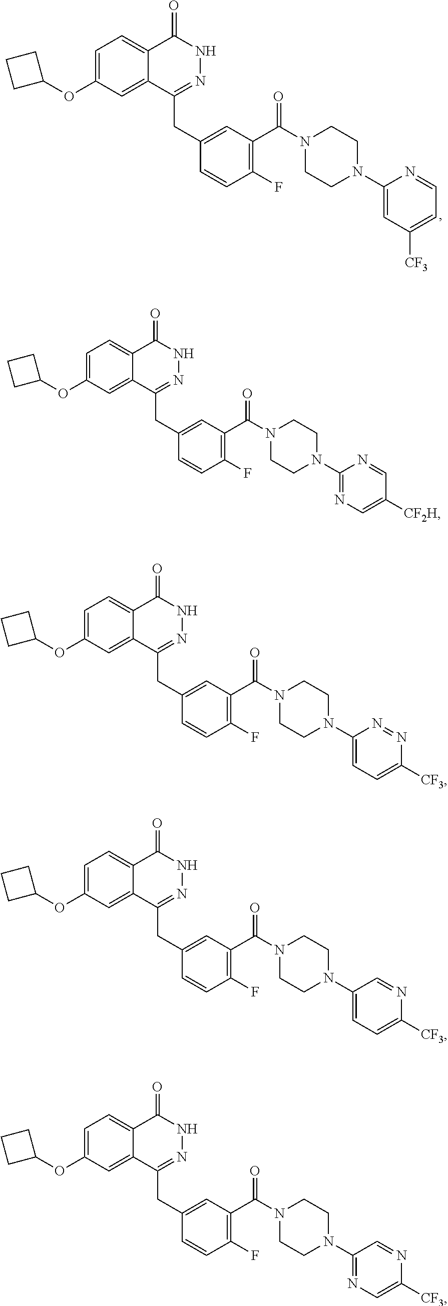 [SMILES: O=C(c1cc(Cc2n[nH]c(=O)c3ccc(OC4CCC4)cc23)ccc1F)N1CCN(c2cc(C(F)(F)F)ccn2)CC1 ; O=C(c1cc(Cc2n[nH]c(=O)c3ccc(OC4CCC4)cc23)ccc1F)N1CCN(c2ncc(C(F)F)cn2)CC1 ; O=C(c1cc(Cc2n[nH]c(=O)c3ccc(OC4CCC4)cc23)ccc1F)N1CCN(c2ccc(C(F)(F)F)nn2)CC1 ; O=C(c1cc(Cc2n[nH]c(=O)c3ccc(OC4CCC4)cc23)ccc1F)N1CCN(c2ccc(C(F)(F)F)nc2)CC1 ; O=C(c1cc(Cc2n[nH]c(=O)c3ccc(OC4CCC4)cc23)ccc1F)N1CCN(c2cnc(C(F)(F)F)cn2)CC1]

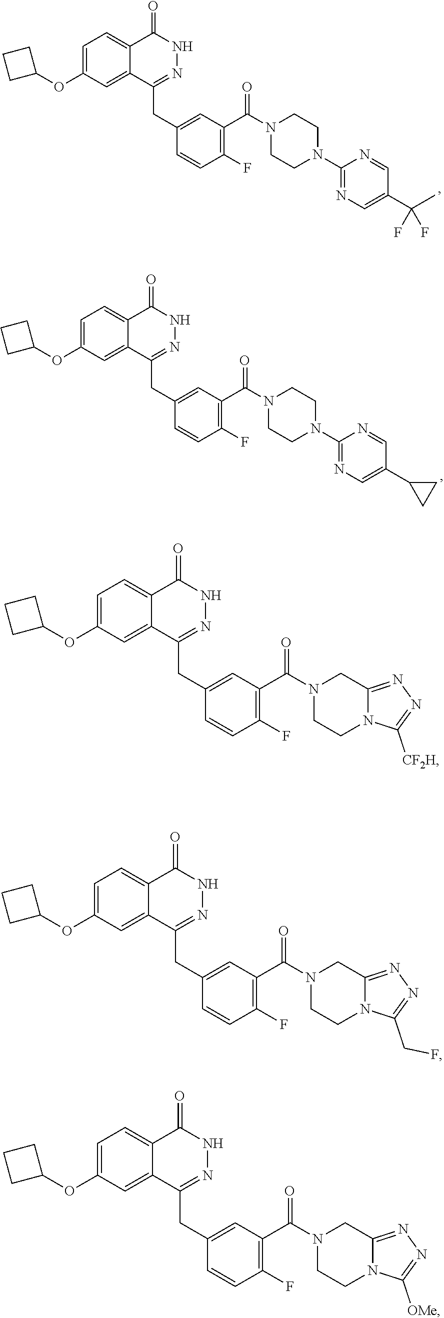 [SMILES: CC(F)(F)c1cnc(N2CCN(C(=O)c3cc(Cc4n[nH]c(=O)c5ccc(OC6CCC6)cc45)ccc3F)CC2)nc1 ; O=C(c1cc(Cc2n[nH]c(=O)c3ccc(OC4CCC4)cc23)ccc1F)N1CCN(c2ncc(C3CC3)cn2)CC1 ; O=C(c1cc(Cc2n[nH]c(=O)c3ccc(OC4CCC4)cc23)ccc1F)N1CCn2c(nnc2C(F)F)C1 ; O=C(c1cc(Cc2n[nH]c(=O)c3ccc(OC4CCC4)cc23)ccc1F)N1CCn2c(CF)nnc2C1 ; COc1nnc2n1CCN(C(=O)c1cc(Cc3n[nH]c(=O)c4ccc(OC5CCC5)cc34)ccc1F)C2]

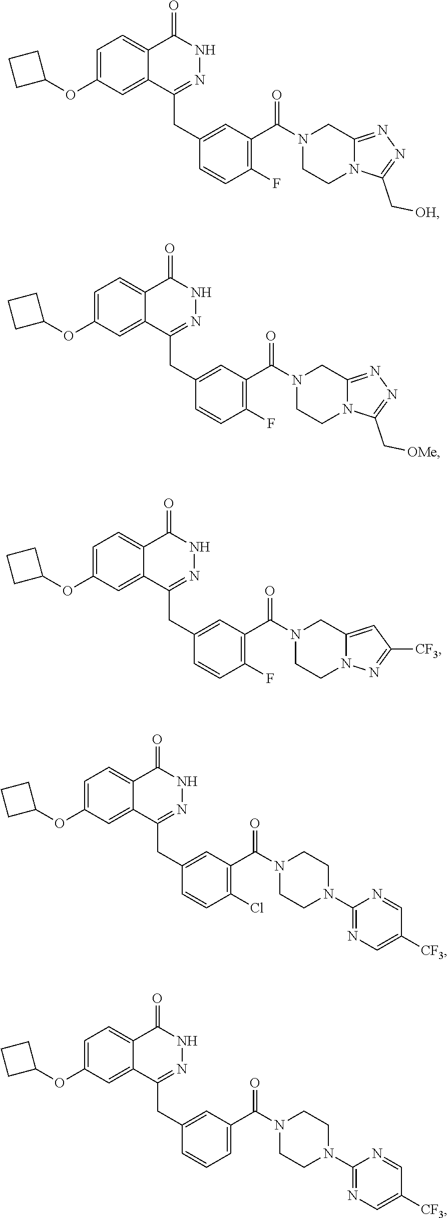 [SMILES: O=C(c1cc(Cc2n[nH]c(=O)c3ccc(OC4CCC4)cc23)ccc1F)N1CCn2c(CO)nnc2C1 ; COCc1nnc2n1CCN(C(=O)c1cc(Cc3n[nH]c(=O)c4ccc(OC5CCC5)cc34)ccc1F)C2 ; O=C(c1cc(Cc2n[nH]c(=O)c3ccc(OC4CCC4)cc23)ccc1F)N1CCn2nc(C(F)(F)F)cc2C1 ; O=C(c1cc(Cc2n[nH]c(=O)c3ccc(OC4CCC4)cc23)ccc1Cl)N1CCN(c2ncc(C(F)(F)F)cn2)CC1 ; O=C(c1cccc(Cc2n[nH]c(=O)c3ccc(OC4CCC4)cc23)c1)N1CCN(c2ncc(C(F)(F)F)cn2)CC1]

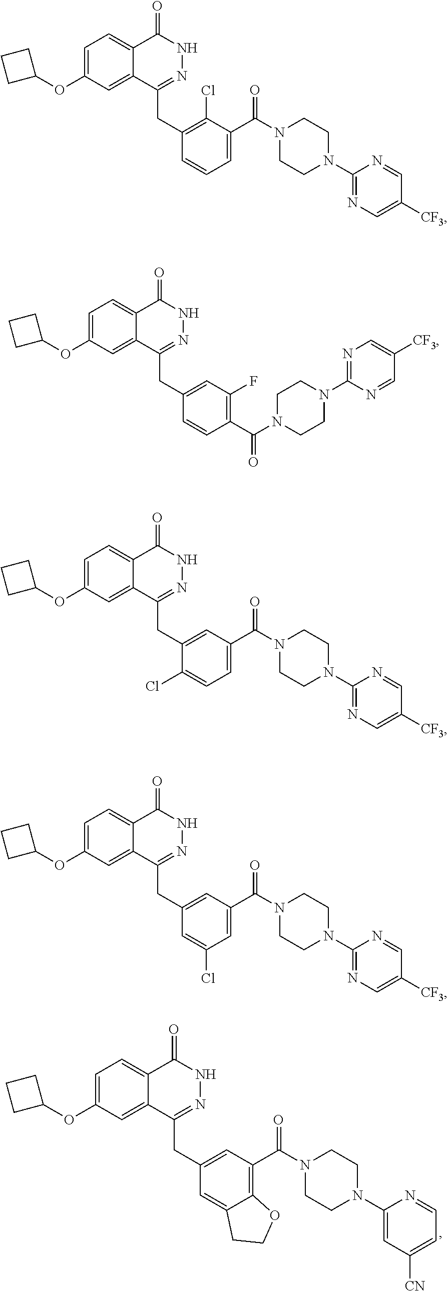 [SMILES: O=C(c1cccc(Cc2n[nH]c(=O)c3ccc(OC4CCC4)cc23)c1Cl)N1CCN(c2ncc(C(F)(F)F)cn2)CC1 ; O=C(c1ccc(Cc2n[nH]c(=O)c3ccc(OC4CCC4)cc23)cc1F)N1CCN(c2ncc(C(F)(F)F)cn2)CC1 ; O=C(c1ccc(Cl)c(Cc2n[nH]c(=O)c3ccc(OC4CCC4)cc23)c1)N1CCN(c2ncc(C(F)(F)F)cn2)CC1 ; O=C(c1cc(Cl)cc(Cc2n[nH]c(=O)c3ccc(OC4CCC4)cc23)c1)N1CCN(c2ncc(C(F)(F)F)cn2)CC1 ; N#Cc1ccnc(N2CCN(C(=O)c3cc(Cc4n[nH]c(=O)c5ccc(OC6CCC6)cc45)cc4c3OCC4)CC2)c1]

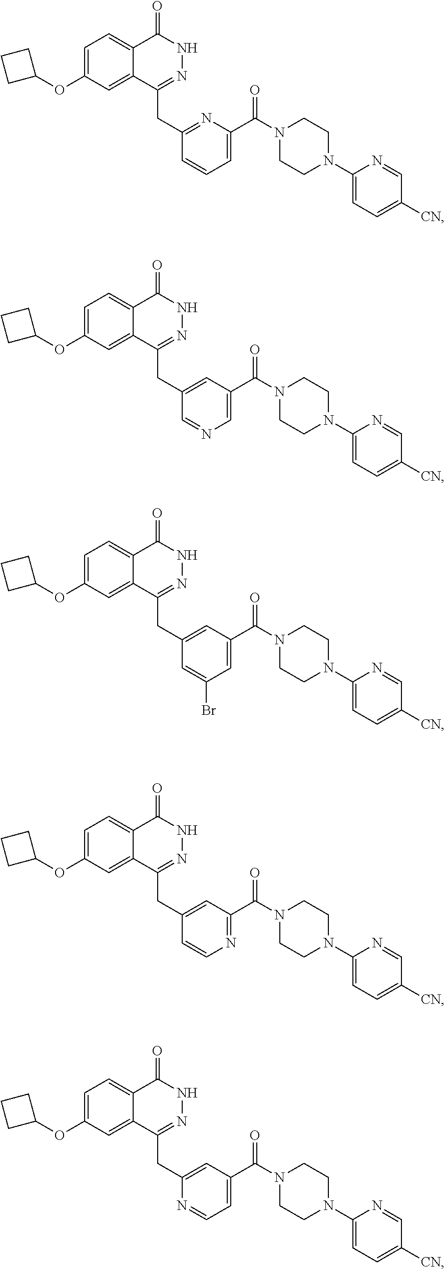 [SMILES: N#Cc1ccc(N2CCN(C(=O)c3cccc(Cc4n[nH]c(=O)c5ccc(OC6CCC6)cc45)n3)CC2)nc1 ; N#Cc1ccc(N2CCN(C(=O)c3cncc(Cc4n[nH]c(=O)c5ccc(OC6CCC6)cc45)c3)CC2)nc1 ; N#Cc1ccc(N2CCN(C(=O)c3cc(Br)cc(Cc4n[nH]c(=O)c5ccc(OC6CCC6)cc45)c3)CC2)nc1 ; N#Cc1ccc(N2CCN(C(=O)c3cc(Cc4n[nH]c(=O)c5ccc(OC6CCC6)cc45)ccn3)CC2)nc1 ; N#Cc1ccc(N2CCN(C(=O)c3ccnc(Cc4n[nH]c(=O)c5ccc(OC6CCC6)cc45)c3)CC2)nc1]

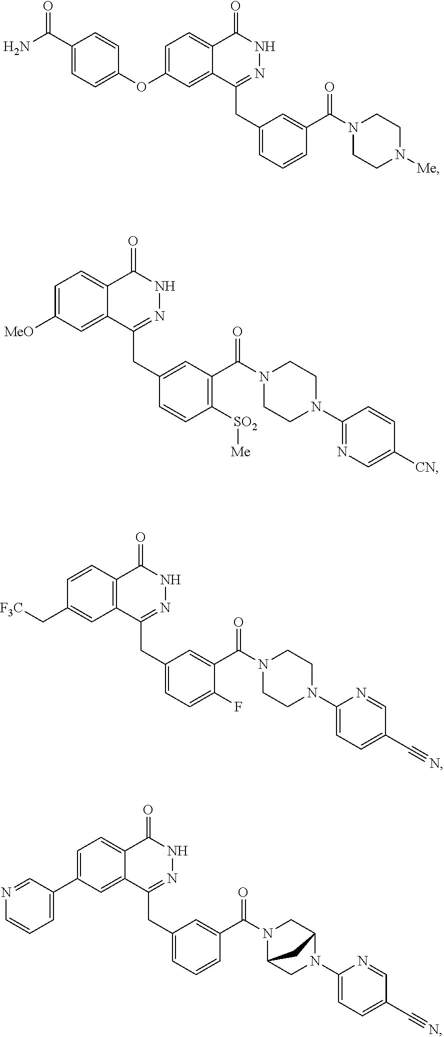 [SMILES: CN1CCN(C(=O)c2cccc(Cc3n[nH]c(=O)c4ccc(Oc5ccc(C(N)=O)cc5)cc34)c2)CC1 ; COc1ccc2c(=O)[nH]nc(Cc3ccc(S(C)(=O)=O)c(C(=O)N4CCN(c5ccc(C#N)cn5)CC4)c3)c2c1 ; N#Cc1ccc(N2CCN(C(=O)c3cc(Cc4n[nH]c(=O)c5ccc(CC(F)(F)F)cc45)ccc3F)CC2)nc1 ; N#Cc1ccc(N2C[C@H]3C[C@@H]2CN3C(=O)c2cccc(Cc3n[nH]c(=O)c4ccc(-c5cccnc5)cc34)c2)nc1]

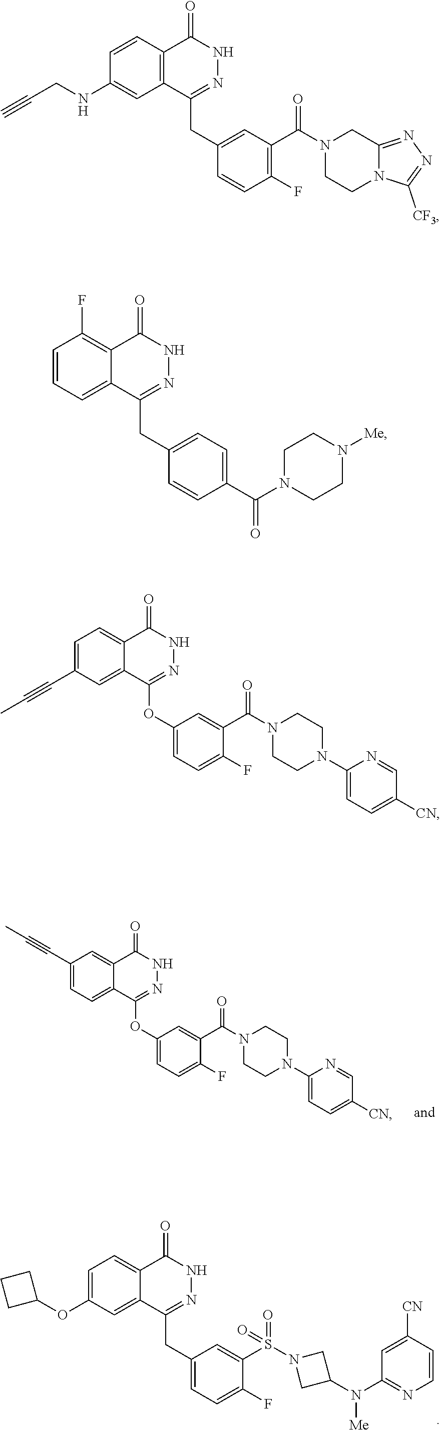 [SMILES: C#CCNc1ccc2c(=O)[nH]nc(Cc3ccc(F)c(C(=O)N4CCn5c(nnc5C(F)(F)F)C4)c3)c2c1 ; CN1CCN(C(=O)c2ccc(Cc3n[nH]c(=O)c4c(F)cccc34)cc2)CC1 ; CC#Cc1ccc2c(=O)[nH]nc(Oc3ccc(F)c(C(=O)N4CCN(c5ccc(C#N)cn5)CC4)c3)c2c1 ; CC#Cc1ccc2c(Oc3ccc(F)c(C(=O)N4CCN(c5ccc(C#N)cn5)CC4)c3)n[nH]c(=O)c2c1 ; CN(c1cc(C#N)ccn1)C1CN(S(=O)(=O)c2cc(Cc3n[nH]c(=O)c4ccc(OC5CCC5)cc34)ccc2F)C1]

76-89. (canceled)

90. A pharmaceutical composition comprising a compound of claim 4, or a stereoisomer or a pharmaceutically acceptable salt thereof, and a pharmaceutically acceptable carrier.

91-92. (canceled)


SEQUENCE LISTING (9 sequences)

SEQ ID NO: 1 - DNA, 33 bp, synthetic construct
  source 1..33: mol_type = other DNA
gtgagcggctggcggctgttcaagaagattagc

SEQ ID NO: 2 - DNA, 20 bp, synthetic construct
  source 1..20: mol_type = other DNA
ttgtaccgcagctgcaaaat

SEQ ID NO: 3 - DNA, 20 bp, synthetic construct
  source 1..20: mol_type = other DNA
tatattcggcgtttcgggca

SEQ ID NO: 4 - DNA, 19 bp, synthetic construct
  source 1..19: mol_type = other DNA
ctatgcgcgtgttttgcag

SEQ ID NO: 5 - DNA, 21 bp, synthetic construct
  source 1..21: mol_type = other DNA
tctgggtgatgaaggtcgtag

SEQ ID NO: 6 - DNA, 25 bp, synthetic construct
  source 1..25: mol_type = other DNA
tggtctcccttctctacactcttgt

SEQ ID NO: 7 - DNA, 31 bp, synthetic construct
  source 1..31: mol_type = other DNA
attttccctattacattaaatcaatggttct

SEQ ID NO: 8 - DNA, 20 bp, synthetic construct
  source 1..20: mol_type = other DNA
tgggagatgtcctcaactgc

SEQ ID NO: 9 - DNA, 20 bp, synthetic construct
  source 1..20: mol_type = other DNA
ccaggagtagctgttgtact